(19) United States
(12) Patent Application Publication
(10) Pub. No.: US 2022/0356185 A1
(43) Pub. Date: Nov. 10, 2022

(54) MERTK DEGRADERS AND USES THEREOF

(71) Applicant: Kymera Therapeutics, Inc., Watertown, MA (US)

(72) Inventors: Nan Ji, Arlington, MA (US); Nello MAINOLFI, Belmont, MA (US); Matthew M. WEISS, Boston, MA (US)

(21) Appl. No.: 17/258,344

(22) PCT Filed: Jul. 3, 2019
(86) PCT No.: PCT/US2019/040520

§ 371 Date: Jan. 6, 2021

Related U.S. Application Data
(60) Provisional application No. 62/860,505, filed on Jun. 12, 2019.
(60) Provisional application No. 62/831,029, filed on Apr. 8, 2019.
(60) Provisional application No. 62/694,958, filed on Jul. 6, 2018.

(51) Int. Cl. C07D 487/04; C07D 401/14; C07D 519/00; C07D 417/14; C07D 401/04

(52) U.S. Cl. CPC C07D 487/04; C07D 401/14; C07D 401/04; C07D 417/14; C07D 519/00

(57) ABSTRACT

The present invention provides compounds, compositions thereof, and methods of using the same.

### TECHNICAL FIELD OF THE INVENTION

The present invention relates to compounds and methods useful for the modulation of members of the TAM receptor kinase family (i.e., Tyro3, Ax1, and Mer) via ubiquitination and/or degradation by compounds according to the present invention. The invention also provides pharmaceutically acceptable compositions comprising compounds of the present invention and methods of using said compositions in the treatment of various disorders.

### BACKGROUND OF THE INVENTION

Ubiquitin-Proteasome Pathway (UPP) is a critical pathway that regulates key regulator proteins and degrades misfolded or abnormal proteins. UPP is central to multiple cellular processes, and if defective or imbalanced, it leads to pathogenesis of a variety of diseases. The covalent attachment of ubiquitin to specific protein substrates is achieved through the action of E3 ubiquitin ligases.

There are over 600 E3 ubiquitin ligases which facilitate the ubiquitination of different proteins in vivo, which can be divided into four families: HECT-domain E3s, U-box E3s, monomeric RING E3s and multi-subunit E3s (see generally Li, W. et al., Genome-wide and functional annotation of human E3 ubiquitin ligases identifies MULAN, a mitochondrial E3 that regulates the organelle's dynamics and signaling, PLOS One (2008) 3(1): e1487; Berndsen, C. E. et al., New insights into ubiquitin E3 ligase mechanism, Nat. Struct. Mol. Biol. (2014) 21(4): 301-7; Deshaies, R. J. et al., RING domain E3 ubiquitin ligases, Ann. Rev. Biochem. (2009) 78: 399-434; Spratt, D. E. et al., RBR E3 ubiquitin ligases: new structures, new insights, new questions, Biochem. (2014) 458(3): 421-37; and Wang, Z. et al., Roles of F-box proteins in cancer, Nat. Rev. Cancer. (2014) 14(4) 233-347).

UPP plays a key role in the degradation of short-lived and regulatory proteins important in a variety of basic cellular processes, including regulation of the cell cycle, modulation of cell surface receptors and ion channels, and antigen presentation. The pathway has been implicated in several forms of malignancy, in the pathogenesis of several genetic diseases (e.g., cystic fibrosis, Angelman's syndrome, and Liddle syndrome), in immune surveillance/viral pathogenesis, and in the pathology of muscle wasting. Many diseases are associated with an abnormal UPP and negatively affect cell cycle and division, the cellular response to stress and to extracellular modulators, morphogenesis of neuronal networks, modulation of cell surface receptors, ion channels, the secretory pathway, DNA repair and biogenesis of organelles.

Aberrations in the process have recently been implicated in the pathogenesis of several diseases, both inherited and acquired. These diseases fall into two major groups: (a) those that result from loss of function with the resultant stabilization of certain proteins, and (b) those that result from gain of function, i.e., abnormal or accelerated degradation of the protein target.

The UPP is used to induce selective protein degradation, including use of fusion proteins to artificially ubiquitinate target proteins and synthetic small-molecule probes to induce proteasome-dependent degradation. Bifunctional compounds composed of a target protein-binding ligand and an E3 ubiquitin ligase ligand, induced proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful as biochemical reagents and lead to a new paradigm for the treatment of diseases by removing pathogenic or oncogenic proteins (C. M. Crews, Chem. Biol., 2010, 17(6):551-5; and J. S. Schnnekloth Jr. & C. M. Crews, Chembiochem, 2005, 6(1): 40-6).

An ongoing need exists in the art for effective treatments for disease, especially hyperplasias and cancers, such as multiple myeloma. However, non-specific effects, and the inability to target and modulate certain classes of proteins altogether, such as transcription factors, remain as obstacles to the development of effective anti-cancer agents. As such, small molecule therapeutic agents that leverage E3 ligase mediated protein degradation to target cancer-associated proteins such as members of the TAM receptor kinase family, including Tyro3, Axl, and Mer tyrosine kinase (“MerTK”), hold promise as therapeutic agents. Accordingly, there remains a need to find bifunctional compounds that are Tyro3, Axl, and/or MerTK degraders useful as therapeutic agents.

### SUMMARY OF THE INVENTION

The present application relates novel bifunctional compounds, which function to recruit TAM receptor kinases to E3 Ubiquitin Ligase for degradation, and methods of preparation and uses thereof. In particular, the present disclosure provides bifunctional compounds, which find utility as modulators of targeted ubiquitination of Tyro3, Axl, and/or MerTK, which are then degraded and/or otherwise inhibited by the bifunctional compounds as described herein. An advantage of the compounds provided herein is that a broad range of pharmacological activities is possible, consistent with the degradation/inhibition of Tyro3, Axl, and/or MerTK. In addition, the description provides methods of using an effective amount of the compounds as described herein for the treatment or amelioration of a disease condition, such as cancer, e.g., multiple myeloma.

The present application further relates to targeted degradation of one or more TAM receptor kinases through the use of bifunctional molecules, including bifunctional molecules that link a cereblon-binding moiety to a ligand that binds Tyro3, Axl, and/or MerTK.

It has now been found that compounds of this invention, and pharmaceutically acceptable compositions thereof, are effective as degraders of one or more TAM receptor kinases. Such compounds have the general Formula I:

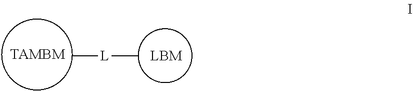

or a pharmaceutically acceptable salt thereof, wherein each variable is as defined and described herein.

It has also now been found that compounds of this invention, and pharmaceutically acceptable compositions thereof, are effective as degraders of one or more TAM receptor kinases. Such compounds have the general Formula VI:

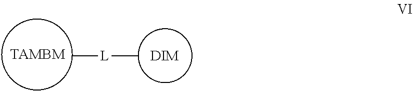

or a pharmaceutically acceptable salt thereof, wherein each variable is as defined and described herein.

Compounds of the present invention, and pharmaceutically acceptable compositions thereof, are useful for treating a variety of diseases, disorders or conditions, associated with regulation of signaling pathways implicating a TAM receptor kinase (e.g., MerTK). Such diseases, disorders, or conditions include those described herein.

Compounds provided by this invention are also useful for the study of the TAM receptor kinase (e.g., MerTK) enzymes in biological and pathological phenomena; the study of intracellular signal transduction pathways occurring in bodily tissues; and the comparative evaluation of new Tryo3, Axl, MerTK inhibitors or Tryo3, Axl, MerTK degraders or other regulators of kinases, signaling pathways, and cytokine levels in vitro or in vivo.

### DETAILED DESCRIPTION OF CERTAIN EMBODIMENTS

### 1. General Description of Certain Embodiments of the Invention:

Compounds of the present invention, and compositions thereof, are useful as degraders and/or inhibitors of TAM receptor kinases. In some embodiments, a provided compound degrades and/or inhibits MerTK.

In certain embodiments, the present invention provides a compound of Formula I:

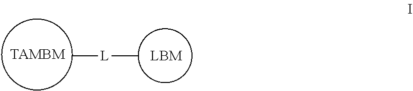

or a pharmaceutically acceptable salt thereof, wherein:

TAMBM is a TAM receptor kinase binding moiety;

L is a bivalent moiety that connects TAMBM to LBM; and

LBM is a ligase binding moiety.

In certain embodiments, the present invention provides a compound of Formula VI:

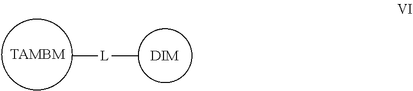

or a pharmaceutically acceptable salt thereof, wherein:

TAMBM is a TAM receptor kinase binding moiety;

L is a bivalent moiety that connects TAMBM to DIM; and

DIM is a degradation inducing moiety selected from LBM, a lysine mimetic, and hydrogen.

### 2. Compounds and Definitions:

Compounds of the present invention include those described generally herein, and are further illustrated by the classes, subclasses, and species disclosed herein. As used herein, the following definitions shall apply unless otherwise indicated. For purposes of this invention, the chemical elements are identified in accordance with the Periodic Table of the Elements, CAS version, Handbook of Chemistry and Physics, 75^{th} Ed. Additionally, general principles of organic chemistry are described in “Organic Chemistry”, Thomas Sorrell, University Science Books, Sausalito: 1999, and “March's Advanced Organic Chemistry”, 5^{th} Ed., Ed.: Smith, M. B. and March, J., John Wiley & Sons, New York: 2001, the entire contents of which are hereby incorporated by reference.

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle,” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

As used herein, the term “bridged bicyclic” refers to any bicyclic ring system, i.e., carbocyclic or heterocyclic, saturated or partially unsaturated, having at least one bridge. As defined by IUPAC, a “bridge” is an unbranched chain of atoms or an atom or a valence bond connecting two bridgeheads, where a “bridgehead” is any skeletal atom of the ring system which is bonded to three or more skeletal atoms (excluding hydrogen). In some embodiments, a bridged bicyclic group has 7-12 ring members and 0-4 heteroatoms independently selected from nitrogen, oxygen, or sulfur. Such bridged bicyclic groups are well known in the art and include those groups set forth below where each group is attached to the rest of the molecule at any substitutable carbon or nitrogen atom. Unless otherwise specified, a bridged bicyclic group is optionally substituted with one or more substituents as set forth for aliphatic groups. Additionally or alternatively, any substitutable nitrogen of a bridged bicyclic group is optionally substituted. Exemplary bridged bicyclics include:

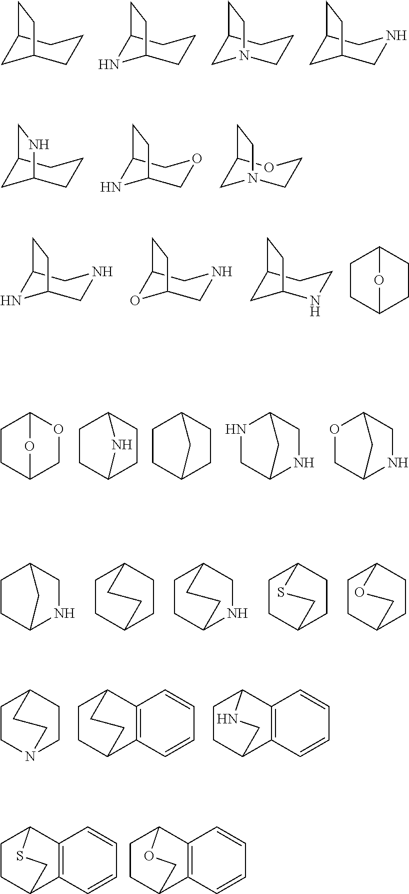

The term “lower alkyl” refers to a C₁₋₄ straight or branched alkyl group. Exemplary lower alkyl groups are methyl, ethyl, propyl, isopropyl, butyl, isobutyl, and tert-butyl.

The term “lower haloalkyl” refers to a C₁₋₄ straight or branched alkyl group that is substituted with one or more halogen atoms.

The term “heteroatom” means one or more of oxygen, sulfur, nitrogen, phosphorus, or silicon (including, any oxidized form of nitrogen, sulfur, phosphorus, or silicon; the quaternized form of any basic nitrogen or; a substitutable nitrogen of a heterocyclic ring, for example N (as in 3,4-dihydro-2H-pyrrolyl), NH (as in pyrrolidinyl) or NR⁺ (as in N-substituted pyrrolidinyl)).

The term “unsaturated,” as used herein, means that a moiety has one or more units of unsaturation.

As used herein, the term “bivalent C₁₋₈ (or C₁₋₆) saturated or unsaturated, straight or branched, hydrocarbon chain”, refers to bivalent alkylene, alkenylene, and alkynylene chains that are straight or branched as defined herein.

The term “alkylene” refers to a bivalent alkyl group. An “alkylene chain” is a polymethylene group, i.e., —(CH₂)ₙ—, wherein n is a positive integer, preferably from 1 to 6, from 1 to 4, from 1 to 3, from 1 to 2, or from 2 to 3. A substituted alkylene chain is a polymethylene group in which one or more methylene hydrogen atoms are replaced with a substituent. Suitable substituents include those described below for a substituted aliphatic group.

The term “alkenylene” refers to a bivalent alkenyl group. A substituted alkenylene chain is a polymethylene group containing at least one double bond in which one or more hydrogen atoms are replaced with a substituent. Suitable substituents include those described below for a substituted aliphatic group.

As used herein, the term “cyclopropylenyl” refers to a bivalent cyclopropyl group of the following structure:

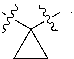

The term “halogen” means F, Cl, Br, or I.

The term “aryl” used alone or as part of a larger moiety as in “aralkyl,” “aralkoxy,” or “aryloxyalkyl,” refers to monocyclic or bicyclic ring systems having a total of five to fourteen ring members, wherein at least one ring in the system is aromatic and wherein each ring in the system contains 3 to 7 ring members. The term “aryl” may be used interchangeably with the term “aryl ring.” In certain embodiments of the present invention, “aryl” refers to an aromatic ring system which includes, but not limited to, phenyl, biphenyl, naphthyl, anthracyl and the like, which may bear one or more substituents. Also included within the scope of the term “aryl,” as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-,” used alone or as part of a larger moiety, e.g., “heteroaralkyl,” or “heteroaralkoxy,” refer to groups having 5 to 10 ring atoms, preferably 5, 6, or 9 ring atoms; having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, i soindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3(4H)-one. A heteroaryl group may be mono- or bicyclic. The term “heteroaryl” may be used interchangeably with the terms “heteroaryl ring,” “heteroaryl group,” or “heteroaromatic,” any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle,” “heterocyclyl,” “heterocyclic radical,” and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as in 3,4-dihydro-2H-pyrrolyl), NH (as in pyrrolidinyl), or ⁺NR (as in N-substituted pyrrolidinyl).

A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle,” “heterocyclyl,” “heterocyclyl ring,” “heterocyclic group,” “heterocyclic moiety,” and “heterocyclic radical,” are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted,” whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable,” as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{o}; —(CH₂)₀₋₄OR^{o}; —O(CH₂)₀₋₄R^{o}, —O—(CH₂)₀₋₄C(O)OR^{o}; —(CH₂)₀₋₄CH(OR^{o})₂; —(CH₂)_{0-4l SR}^{o}; —(CH₂)₀₋₄Ph, which may be substituted with R^{o}; —(CH₂)₀₋₄O(CH₂)₀₋₁Ph which may be substituted with R^{o}; —CH═CHPh, which may be substituted with R^{o}; —(CH₂)₀₋₄O(CH₂)₀₋₁-pyridyl which may be substituted with R^{o}; —NO₂; —CN; —N₃; —(CH₂)₀₋₄N(R^{o})₂; —(CH₂)₀₋₄N(R^{o}C(O)R^{o}; —N(R^{o}C(S)R^{o}; —(CH₂)₀₋₄N(R^{o})C(O)NR^{o}₂; —N(R^{o}C(S)NR^{o}₂; —(CH₂)₀₋₄N(R^{o})C(O)OR^{o}; —N(R^{o})N(R^{o})C(O)R^{o}; —N(R^{o})N(R^{o})C(O)NR^{o}₂; —N(R^{o})N(R^{o})C(O)OR^{o}; —(CH₂)₀₋₄C(O)R^{o}; —C(S)R^{o}; —(CH₂)₀₋₄C(O)OR^{o}; —(CH₂)₀₋₄C(O)SR^{o}; —(CH₂)₀₋₄C(O)OSiR^{o}₃; —(CH₂)₀₋₄OC(O)R^{o}; —OC(O)(CH₂)₀₋₄SR—, SC(S)SR^{o}; —(CH₂)₀₋₄SC(O)R^{o}; —(CH₂)₀₋₄C(O)NR^{o}₂; —C(S)NR^{o}₂; —C(S)SR^{o}; —SC(S)SR^{o}, —(CH₂)₀₋₄OC(O)NR^{o}₂; —C(O)N(OR^{o})R^{o}; —C(O)C(O)R^{o}; —C(O)CH₂C(O)R^{o}; —C(NOR^{o})R^{o}; —(CH₂)₀₋₄ SSR^{o}; —(CH₂)₀₋₄S(O)₂R^{o}; —(CH₂)₀₋₄S(O)₂OR^{o}; —(CH₂)₀₋₄S(O)₂R^{o}; —S(O)₂NR^{o}₂; —(CH₂)₀₋₄S(O)R^{o}; —N(R^{o})S(O)₂NR^{o}₂; —N(R^{o})S(O)₂R^{o}; —N(OR^{o})R^{o}; —C(NH)NR^{o}₂; —P(O)₂R^{o}; —P(O)R^{o}₂; —OP(O)R^{o}₂; —OP(O)(OR^{o}₂; —SiR^{o}₃; —(C₁₋₄ straight or branched alkylene)O—) N(R^{o})₂; or —(C₁₋₄ straight or branched)alkylene)C(O)O—N(R^{o})₂, wherein each R^{o} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{o}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below.

Suitable monovalent substituents on R^{o} (or the ring formed by taking two independent occurrences of R^{o}together with their intervening atoms), are independently halogen, —(CH₂)₀₋₂R¹⁰⁸, -(haloR^{⋅}), —(CH₂)₀₋₂OH, —(CH₂)₀₋₂OR^{⋅}, —(CH₂)₀₋₂CH(OR^{⋅})₂; —O(haloR^{⋅}), —CN, —N₃, —(CH₂)₀₋₂C(O)R^{⋅}, —(CH₂)₀₋₂C(O)OH, —(CH₂)₀₋₂C(O)OR^{⋅}, —(CH₂)₀₋₂SR^{⋅}, —(CH₂)₀₋₂SH, —(CH₂)₀₋₂NH₂, —(CH₂)₀₋₂NHR^{⋅}, —(CH₂)₀₋₂NR^{⋅}₂, —NO₂, —OSiR^{⋅}₃, —C(O)SR^{⋅}, —(C₁₋₄ straight or branched alkylene)C(O)OR^{⋅}, or —SSR^{⋅} wherein each R^{⋅} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{o} include ═O and =S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —O(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include: —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{⋅}, -(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur.

Suitable substituents on the aliphatic group of Rt are independently halogen, —R^{⋅}, —(haloR^{⋅}), —OH, —OR^{⋅}, —O(haloR^{⋅}), —CN, —C(O)OH, —C(O)OR^{⋅}, —NH₂, —NHR^{⋅}, —NR^{⋅}₂, or —NO₂, wherein each R^{⋅} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

As used herein, the term “provided compound” refers to any genus, subgenus, and/or species set forth herein.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention.

Such compounds are useful, for example, as analytical tools, as probes in biological assays, or as therapeutic agents in accordance with the present invention

As used herein, the term “inhibitor” is defined as a compound that binds to and /or inhibits MerTK with measurable affinity. In certain embodiments, an inhibitor has an IC₅₀ and/or binding constant of less than about 50 μM, less than about 1 μM, less than about 500 nM, less than about 100 nM, less than about 10 nM, or less than about 1 nM.

As used herein, the term “degrader” is defined as a heterobifunctional compound that binds to and /or inhibits both at least one TAM receptor kinase (e.g., MerTK) and an E3 ligase with measurable affinity resulting in the ubiqitination and subsequent degradation of the TAM receptor kinase. In some embodiments the TAM receptor kinase is Tyro3. In some embodiments the TAM receptor kinase is Axl. In some embodiments the TAM receptor kinase is MerTK. In certain embodiments, a degrader has an DC₅₀ of less than about 50 μM, less than about 1 μM, less than about 500 nM, less than about 100 nM, less than about 10 nM, or less than about 1 nM.

A compound of the present invention may be tethered to a detectable moiety. It will be appreciated that such compounds are useful as imaging agents. One of ordinary skill in the art will recognize that a detectable moiety may be attached to a provided compound via a suitable substituent. As used herein, the term “suitable substituent” refers to a moiety that is capable of covalent attachment to a detectable moiety. Such moieties are well known to one of ordinary skill in the art and include groups containing, e.g., a carboxylate moiety, an amino moiety, a thiol moiety, or a hydroxyl moiety, to name but a few. It will be appreciated that such moieties may be directly attached to a provided compound or via a tethering group, such as a bivalent saturated or unsaturated hydrocarbon chain. In some embodiments, such moieties may be attached via click chemistry. In some embodiments, such moieties may be attached via a 1,3-cycloaddition of an azide with an alkyne, optionally in the presence of a copper catalyst. Methods of using click chemistry are known in the art and include those described by Rostovtsev et al., Angew. Chem. Int. Ed. 2002, 41, 2596-99 and Sun et al., Bioconjugate Chem., 2006, 17, 52-57.

As used herein, the term “detectable moiety” is used interchangeably with the term “label” and relates to any moiety capable of being detected, e.g., primary labels and secondary labels. Primary labels, such as radioisotopes (e.g., tritium, ³²P, ³³P, ³⁵S, or ¹⁴C), mass-tags, and fluorescent labels are signal generating reporter groups which can be detected without further modifications. Detectable moieties also include luminescent and phosphorescent groups.

The term “secondary label” as used herein refers to moieties such as biotin and various protein antigens that require the presence of a second intermediate for production of a detectable signal. For biotin, the secondary intermediate may include streptavidin-enzyme conjugates. For antigen labels, secondary intermediates may include antibody-enzyme conjugates. Some fluorescent groups act as secondary labels because they transfer energy to another group in the process of nonradiative fluorescent resonance energy transfer (FRET), and the second group produces the detected signal.

The terms “fluorescent label”, “fluorescent dye”, and “fluorophore” as used herein refer to moieties that absorb light energy at a defined excitation wavelength and emit light energy at a different wavelength. Examples of fluorescent labels include, but are not limited to: Alexa Fluor dyes (Alexa Fluor 350, Alexa Fluor 488, Alexa Fluor 532, Alexa Fluor 546, Alexa Fluor 568, Alexa Fluor 594, Alexa Fluor 633, Alexa Fluor 660 and Alexa Fluor 680), AMCA, AMCA-S, BODIPY dyes (BODIPY FL, BODIPY R6G, BODIPY TMR, BODIPY TR, BODIPY 530/550, BODIPY 558/568, BODIPY 564/570, BODIPY 576/589, BODIPY 581/591, BODIPY 630/650, BODIPY 650/665), Carboxyrhodamine 6G, carboxy-X-rhodamine (ROX), Cascade Blue, Cascade Yellow, Coumarin 343, Cyanine dyes (Cy3, Cy5, Cy3.5, Cy5.5), Dansyl, Dapoxyl, Dialkylaminocoumarin, 4′,5′-Dichloro-2′,7′-dimethoxy-fluorescein, DM-NERF, Eosin, Erythrosin, Fluorescein, FAM, Hydroxycoumarin, IRDyes (IRD40, IRD 700, IRD 800), JOE, Lissamine rhodamine B, Marina Blue, Methoxycoumarin, Naphthofluorescein, Oregon Green 488, Oregon Green 500, Oregon Green 514, Pacific Blue, PyMPO, Pyrene, Rhodamine B, Rhodamine 6G, Rhodamine Green, Rhodamine Red, Rhodol Green, 2′,4′,5′,7′-Tetra-bromosulfone-fluorescein, Tetramethyl-rhodamine (TMR), Carboxytetramethylrhodamine (TAMRA), Texas Red, Texas Red-X.

The term “mass-tag” as used herein refers to any moiety that is capable of being uniquely detected by virtue of its mass using mass spectrometry (MS) detection techniques. Examples of mass-tags include electrophore release tags such as N-[3-[4′-[(p-Methoxytetrafluorobenzyl)oxy]phenyl]-3-methylglyceronyl]isonipecotic Acid, 4′-[2,3,5,6-Tetrafluoro-4-(pentafluorophenoxyl)]methyl acetophenone, and their derivatives. The synthesis and utility of these mass-tags is described in U.S. Pat. Nos. 4,650,750, 4,709,016, 5,360,8191, 5,516,931, 5,602,273, 5,604,104, 5,610,020, and 5,650,270. Other examples of mass-tags include, but are not limited to, nucleotides, dideoxynucleotides, oligonucleotides of varying length and base composition, oligopeptides, oligosaccharides, and other synthetic polymers of varying length and monomer composition. A large variety of organic molecules, both neutral and charged (biomolecules or synthetic compounds) of an appropriate mass range (100-2000 Daltons) may also be used as mass-tags.

The terms “measurable affinity” and “measurably inhibit,” as used herein, means a measurable change in a TAM receptor kinase family member's activity between a sample comprising a compound of the present invention, or composition thereof, and the TAM receptor kinase, and an equivalent sample comprising the TAM receptor kinase, in the absence of said compound, or composition thereof.

### 3. Description of Exemplary Embodiments:

As described above, in certain embodiments, the present invention provides a compound of Formula I:

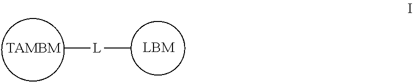

or a pharmaceutically acceptable salt thereof, wherein:

TAMBM is a TAM receptor kinase binding moiety;

L is a bivalent moiety that connects TAMBM to LBM; and

LBM is a ligase binding moiety.

Ligase Binding Moiety (LBM)

In some embodiments, LBM is an E3 ligase ligand. Such E3 ligase ligands are well known to one of ordinary skill in the art and include those described in M. Toure, C. M. Crews, Angew. Chem. Int. Ed. 2016, 55, 1966, T. Uehara et al. Nature Chemical Biology 2017, 13, 675, WO 2017/176708, US 2017/0281784, WO 2017/161119, WO 2017/176957, WO 2017/176958, WO 2015/160845, US 2015/0291562, WO 2016/197032, WO 2016/105518, US 2018/0009779, WO 2017/007612, 2018/0134684, WO 2013/106643, US 2014/0356322, WO 2002/020740, US 2002/0068063, WO 2012/078559, US 2014/0302523, WO 2012/003281, US 2013/0190340, US 2016/0022642, WO 2014/063061, US 2015/0274738, WO 2016/118666, US 2016/0214972, WO 2016/149668, US 2016/0272639, WO 2016/169989, US 2018/0118733, WO 2016/197114, US 2018/0147202, WO 2017/011371, US 2017/0008904, WO 2017/011590, US 2017/0037004, WO 2017/079267, US 2017/0121321, WO 2017/117473, WO 2017/117474, WO 2013/106646, WO 2014/108452, WO 2017/197036, US 2019/0076540, WO 2017/197046, US 2019/0076542, WO 2017/197051, US 2019/0076539, WO 2017/197055, US 2019/0076541, and WO 2017/197056, the entirety of each of which is herein incorporated by reference.

In certain embodiments, the present invention provides a compound of Formula I, wherein LBM is an E3 ubiquitin ligase (cereblon) binding moiety

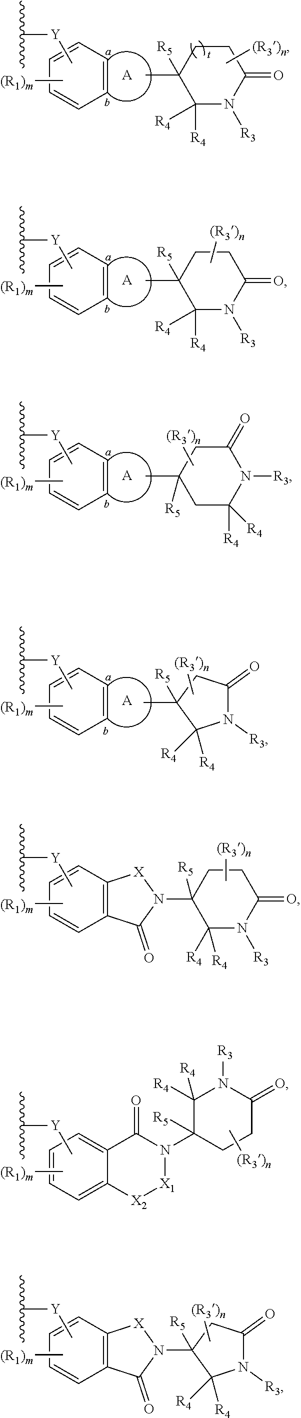

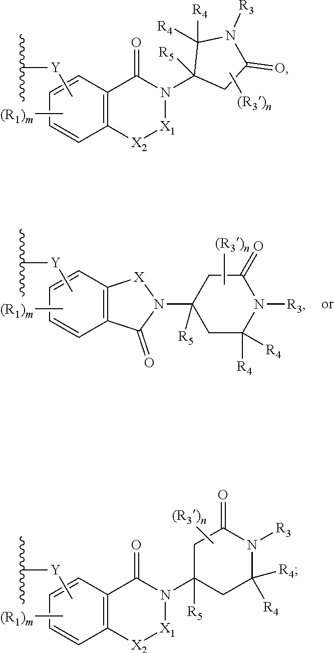

thereby forming a compound of Formulae I-a-1, I-a-2, I-a-3, I-a-4, I-a-5, I-a-6, I-a-7, I-a-8, I-a-9, or I-a-10 respectively:

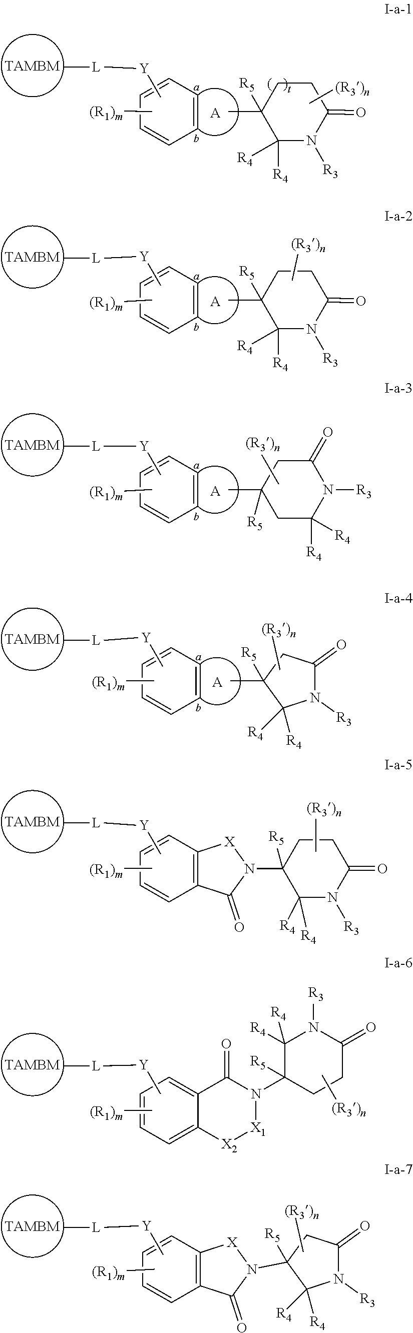

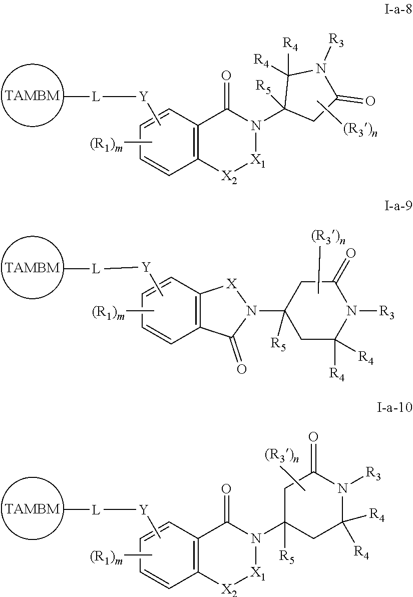

or a pharmaceutically acceptable salt thereof, wherein L and TAMBM are as defined above and described in embodiments herein, and wherein each of the variables

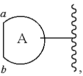

X, X₁, X₂, Y, R₁, R₃, R₃′, R₄, R₅, t, m and n is as defined and described in WO 2017/007612 and US2018/0134684 which is herein incorporated by reference in its entirety.

In certain embodiments, the present invention provides a compound of Formula I, wherein LBM is a VHL E3 ubiquitin ligase binding moiety

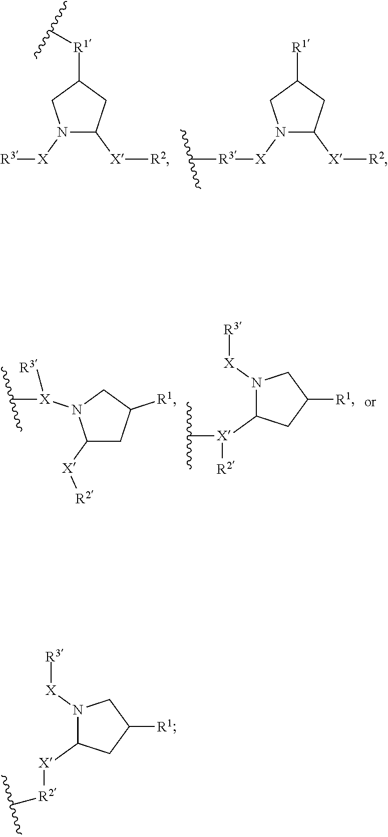

thereby forming a compound of Formulae I-b-1, I-b-2, I-b-3, I-b-4, or I-b-5 respectively:

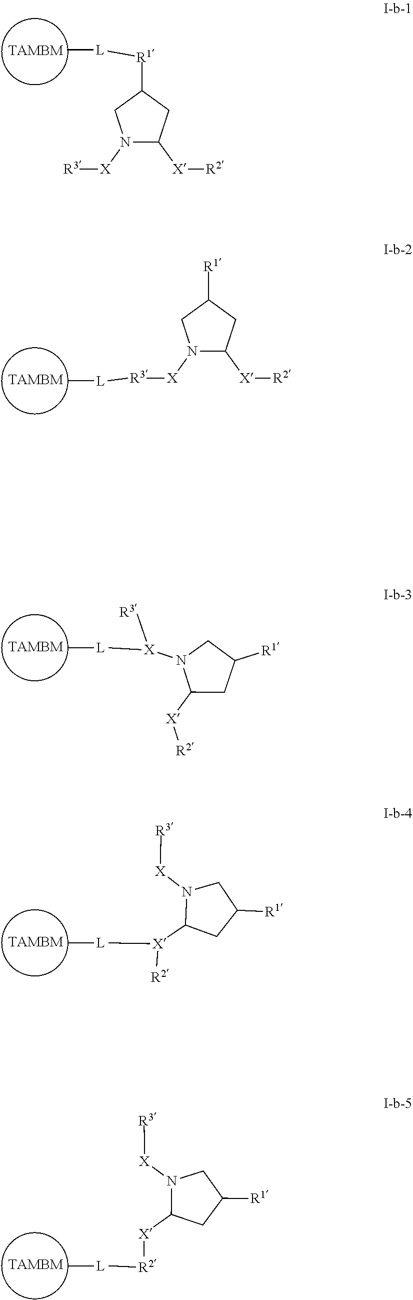

or a pharmaceutically acceptable salt thereof, wherein L and TAMBM are as defined above and described in embodiments herein, and wherein each of the variables R^{1′}, R^{2′}, R^{3′}, X, and X′ is as defined and described in WO 2013/106643 and US 2018/0147202 which is herein incorporated by reference in its entirety.

In certain embodiments, the present invention provides a compound of Formula I, wherein LBM is an E3 ubiquitin ligase (cereblon) binding moiety

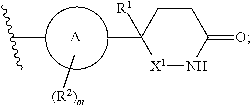

thereby forming a compound of Formula I-c:

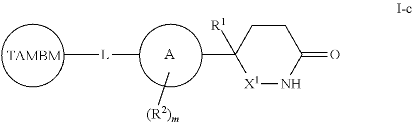

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

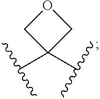 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)CO)OR, —N(R)C(O)R, —N(R)C(O)NR₂, or —N(R)S(O)₂R;
Ring A is a bi- or tricyclic ring selected from

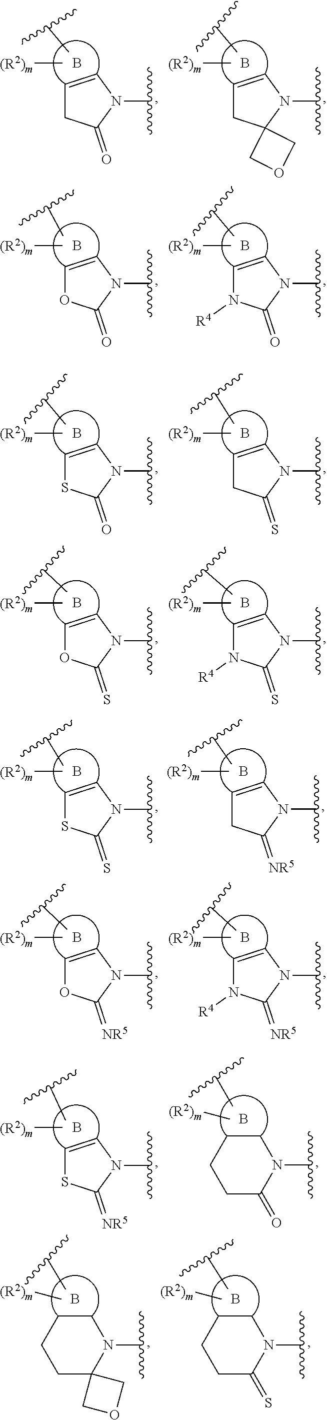

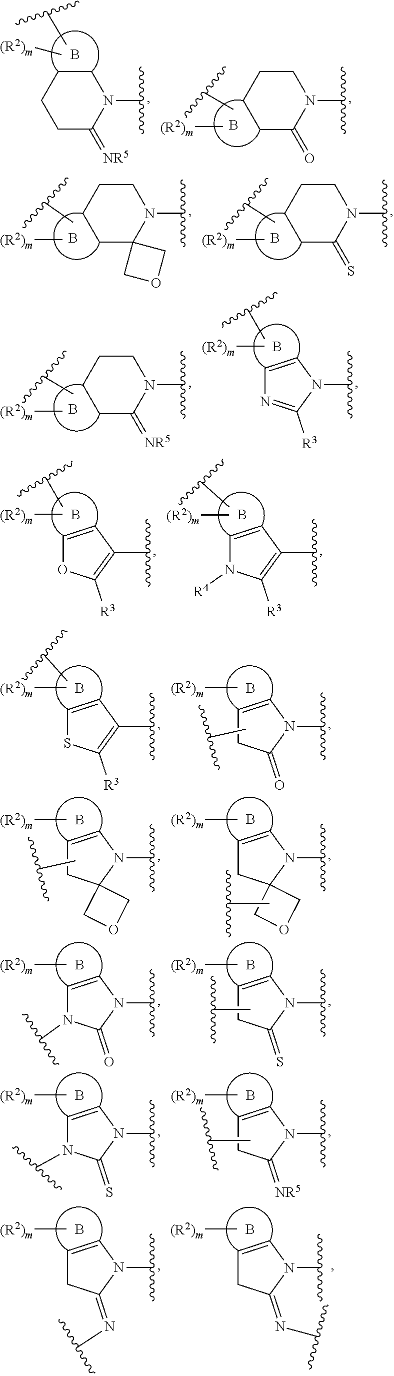

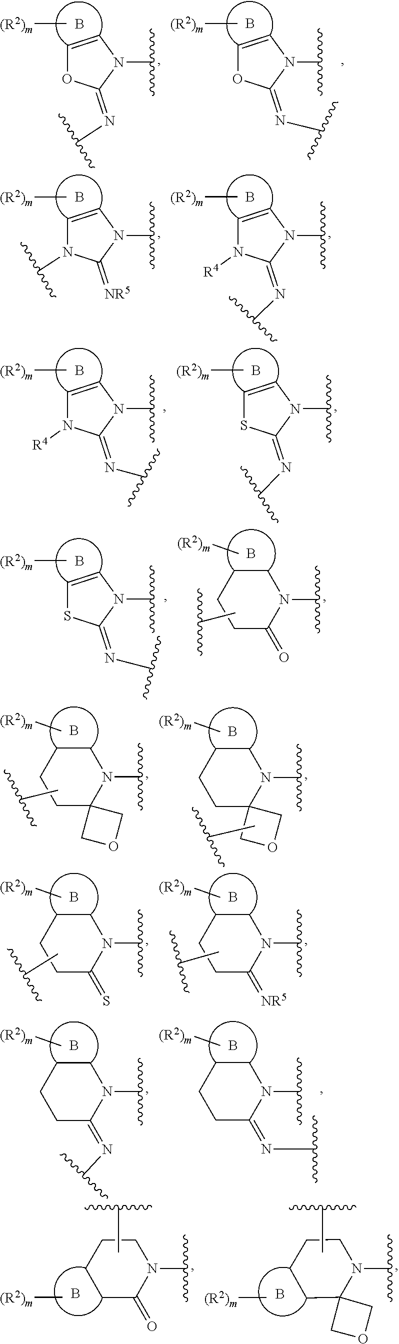

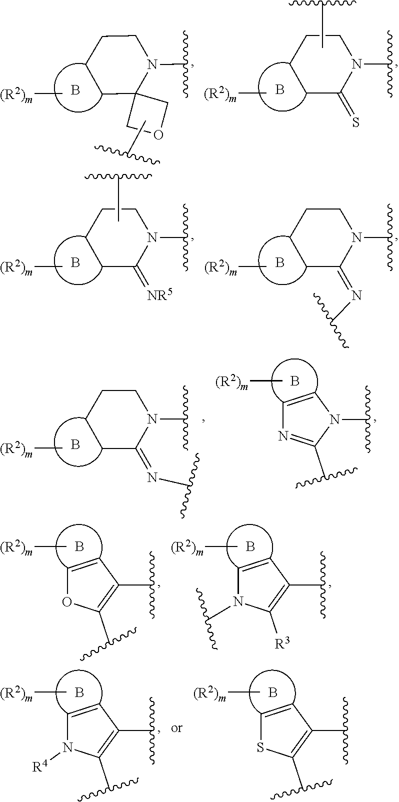

wherein
Ring B is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
   —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of formula I, wherein LBM is an E3 ubiquitin ligase (cereblon) binding moiety thereby forming a compound of formula I-c-1:

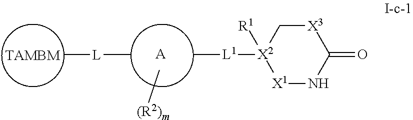

or a pharmaceutically acceptable salt thereof, wherein L and TAMBM are as defined above and described herein, and wherein:
   X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

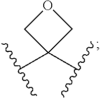 [SMILES: *C1(*)COC1]

X² is a carbon atom or silicon atom;
X³ is a bivalent moiety selected from —CR₂, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, ≥S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from

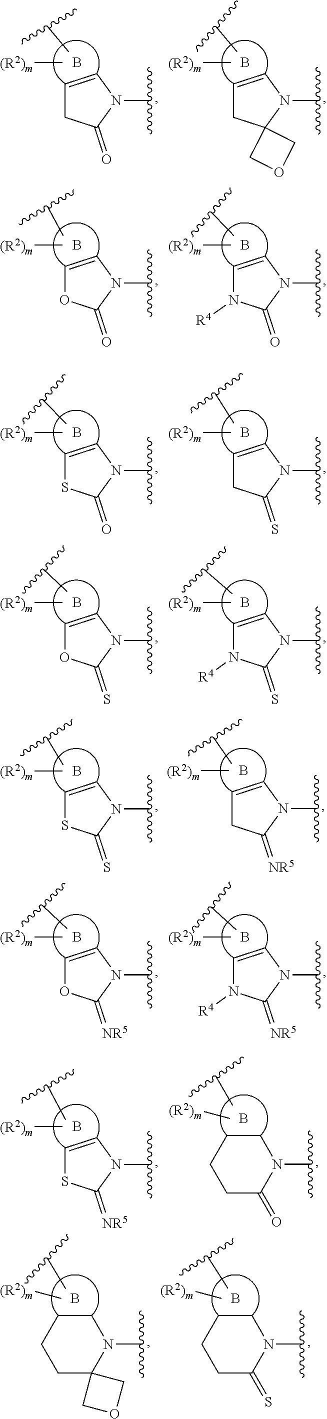

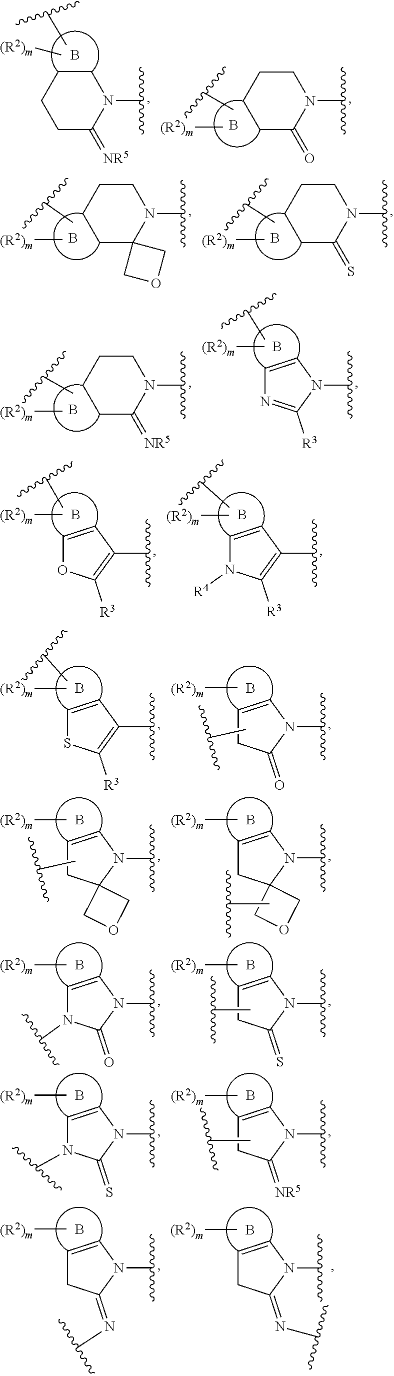

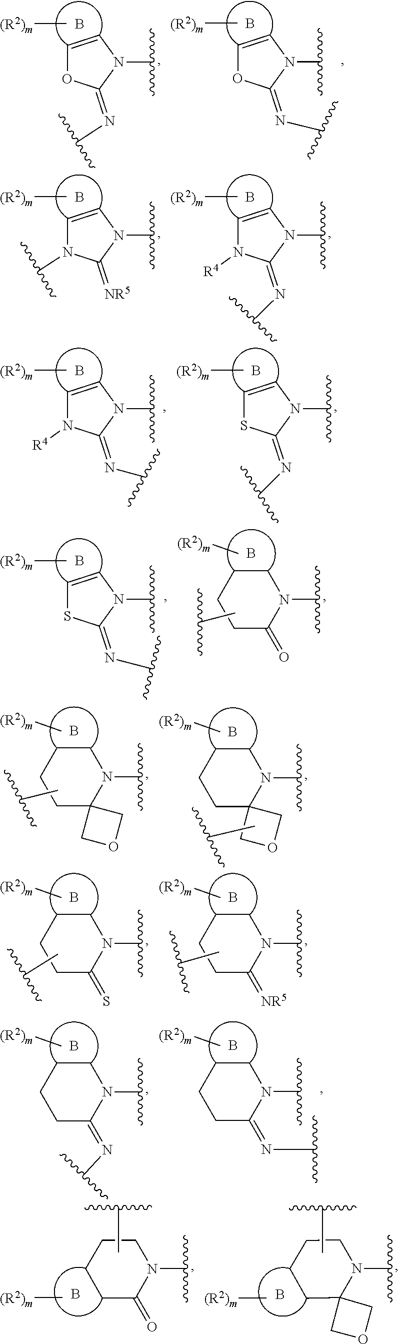

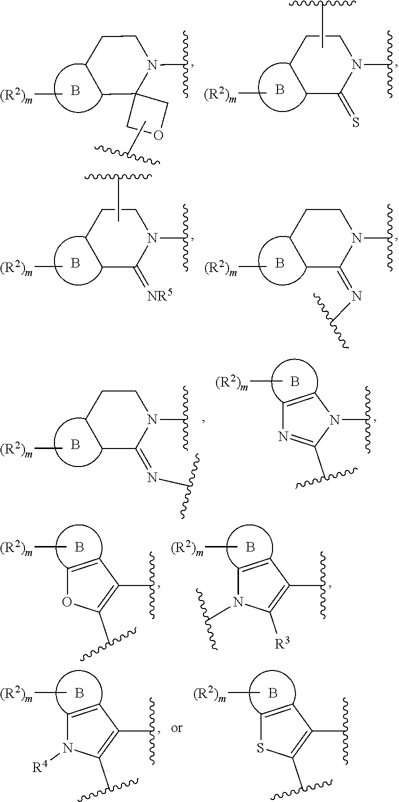

wherein
Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—;
m is 0, 1, 2, 3 or 4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₘ is depicted on Ring B, it is intended, and one of ordinary skill in the art would appreciate, that the point of attachment of —(R²)ₘ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In some embodiments, a compound of formula I-c-1 above is provided as a compound of formula I-c-1′ or formula I-c-1″:

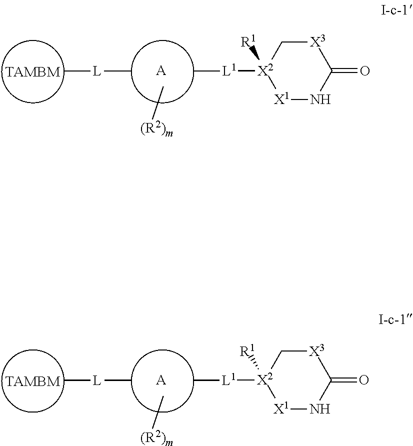

or a pharmaceutically acceptable salt thereof, wherein:
each of TAMBM, Ring A, L, L¹, R¹, R², X¹, X², X³, and m is as defined above.

In certain embodiments, the present invention provides a compound of Formula I, wherein LBM is an E3 ubiquitin ligase (cereblon) binding moiety

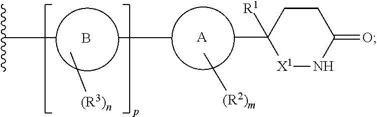

thereby forming a compound of formula I-d:

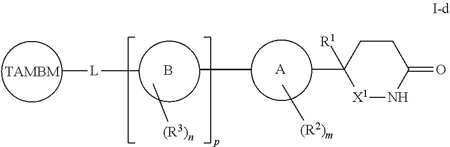

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

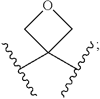 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring A is a mono- or bicyclic ring selected from

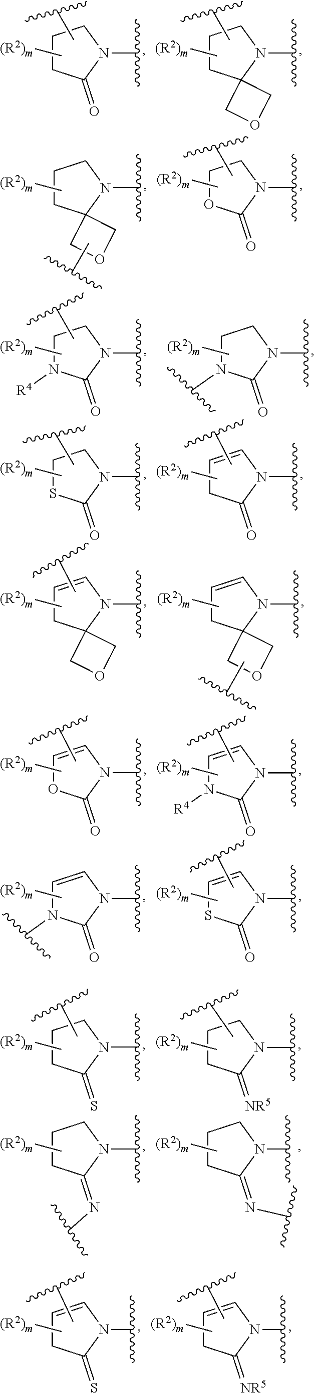

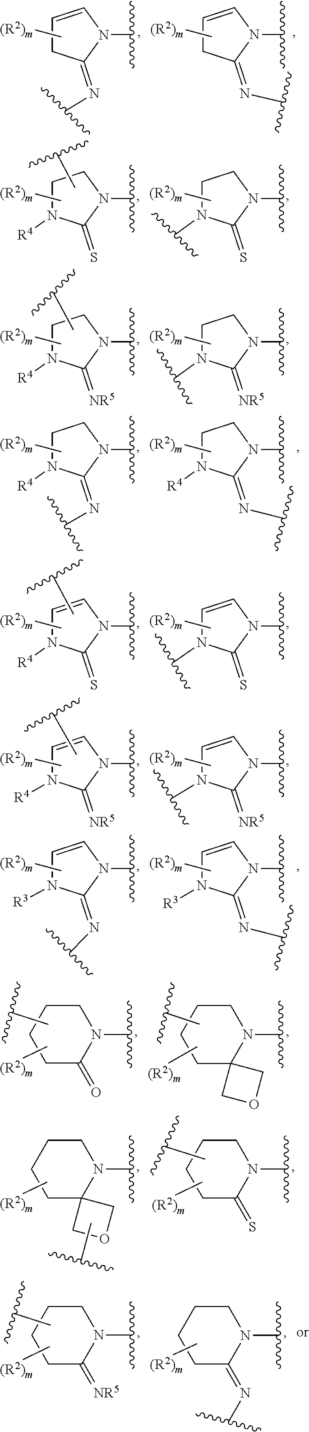

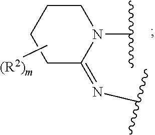

each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1, wherein when p is 0, the bond connecting Ring A and Ring B is connected to

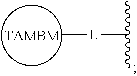

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety thereby forming a compound of formula I-d-1:

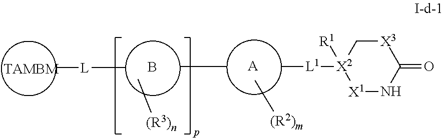

or a pharmaceutically acceptable salt thereof, wherein L and TAMBM are as defined above and described in embodiments herein, and wherein:
   X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

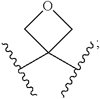 [SMILES: *C1(*)COC1]

X² is a carbon atom or silicon atom;
X³ is a bivalent moiety selected from —CR₂—, —NR—, —O—, —S—, or —Si(R₂)—;
R¹is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a mono- or bicyclic ring selected from

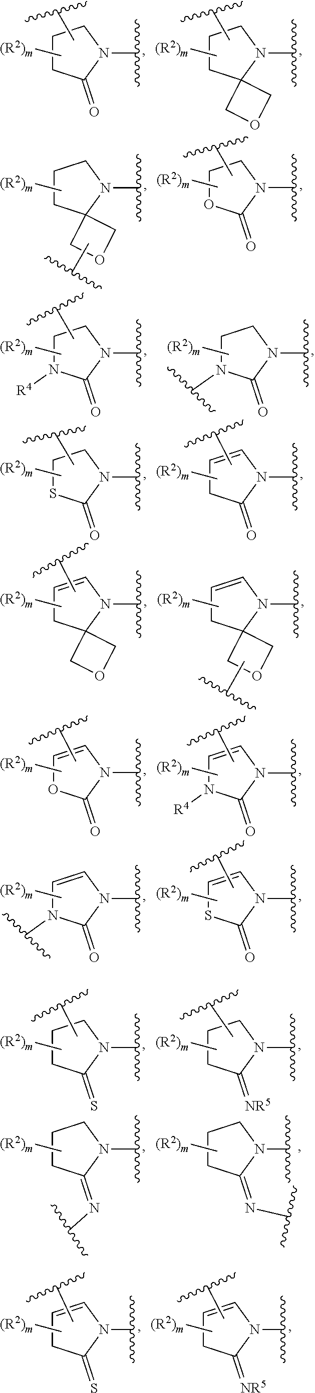

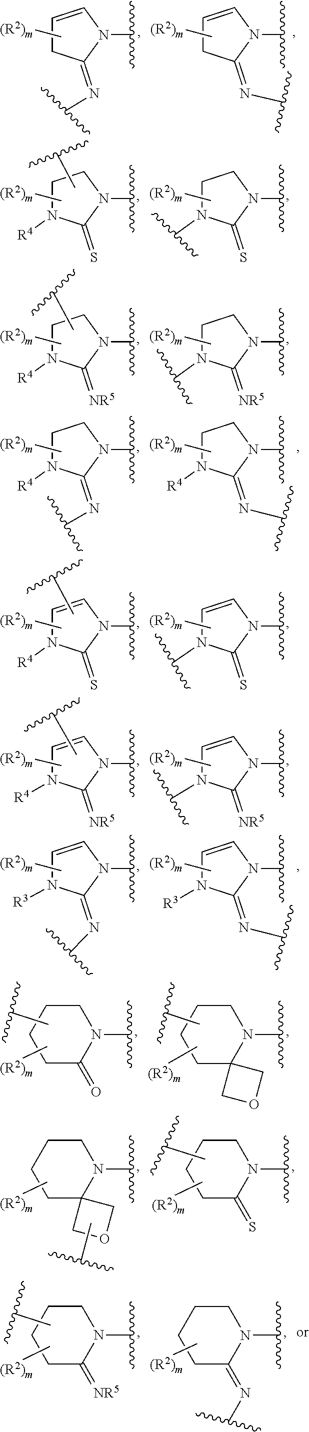

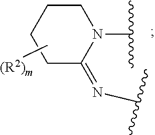

Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(o)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—;
m is 0, 1, or 2;
n is 0, 1, 2, 3, or 4;
p is 0 or 1, wherein when p is 0, the bond connecting Ring A and Ring B is connected to

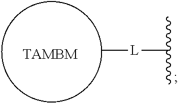

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-d-1 above is provided as a compound of formula I-d-1′ or formula I-d-1″:

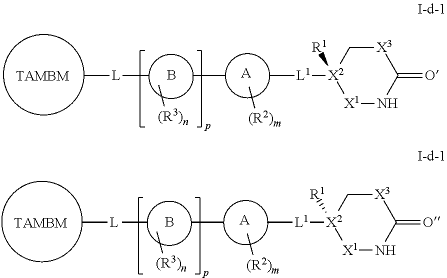

or a pharmaceutically acceptable salt thereof, wherein:
each of TAMBM, Ring A, Ring B, L, L¹, R¹, R², R³, X³, n, p, and m is as defined above.

In certain embodiments, the present invention provides a compound of Formula I, wherein LBM is an E3 ubiquitin ligase (cereblon) binding moiety

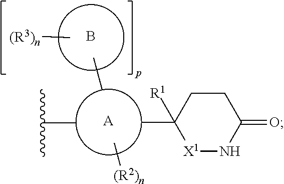

thereby forming a compound of Formula I-e:

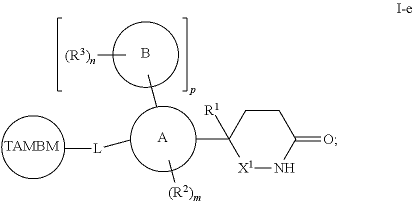

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

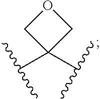 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring A is a mono- or bicyclic ring selected from

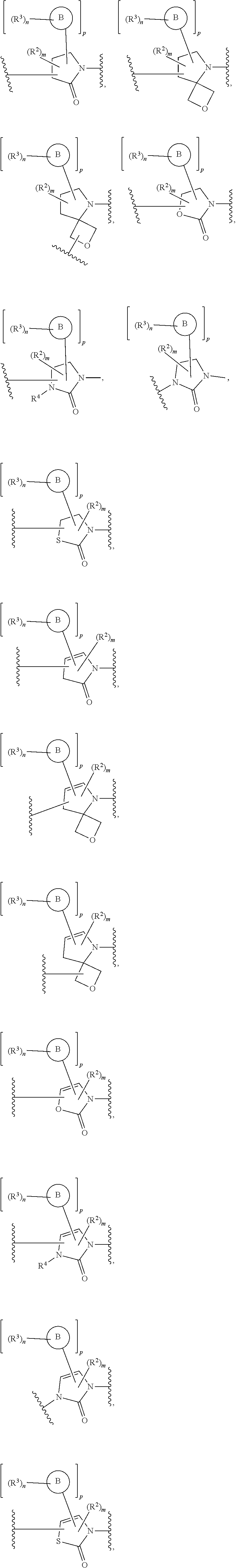

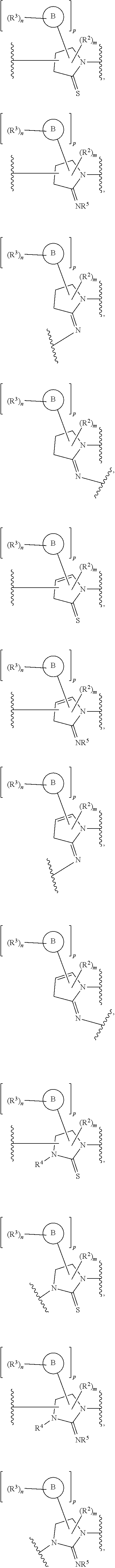

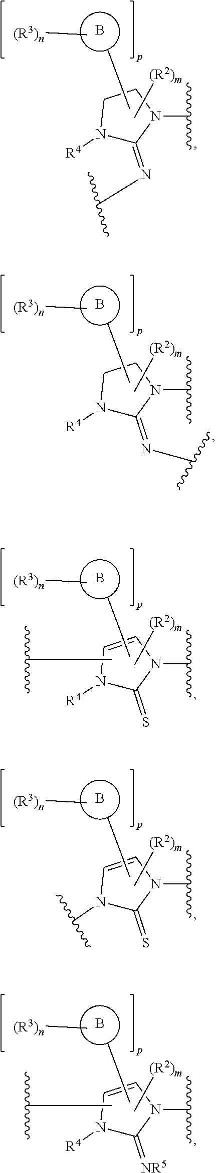

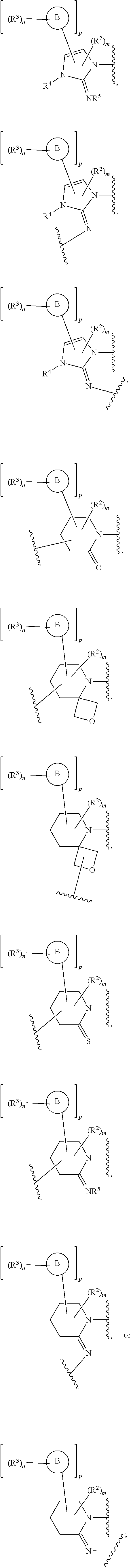

each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
   —N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
   —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety thereby forming a compound of Formula I-e-1:

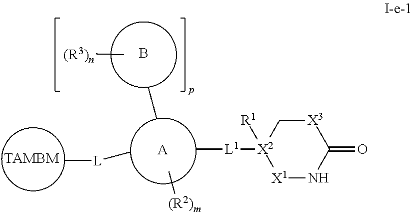

or a pharmaceutically acceptable salt thereof, wherein L and TAMBM are as defined above and described in embodiments herein, and wherein:
   X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

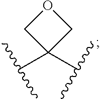 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a mono- or bicyclic ring selected from

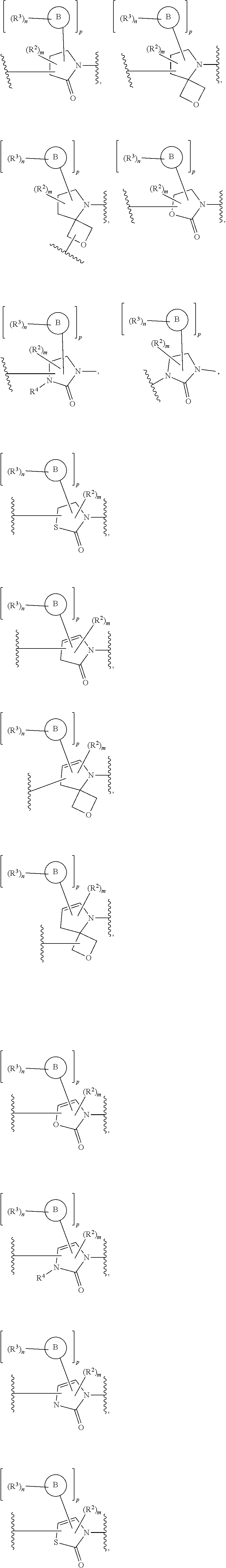

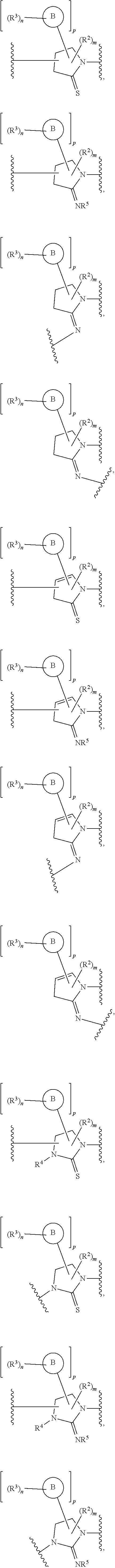

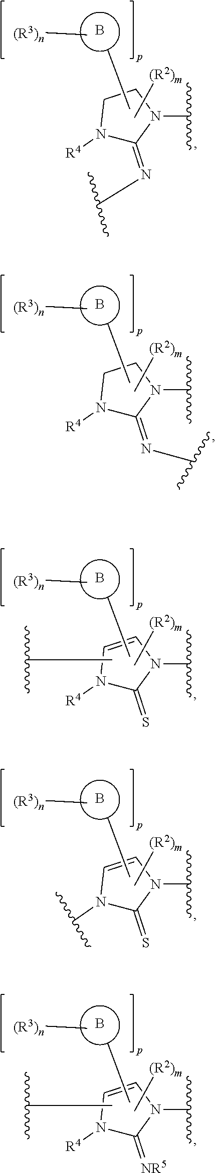

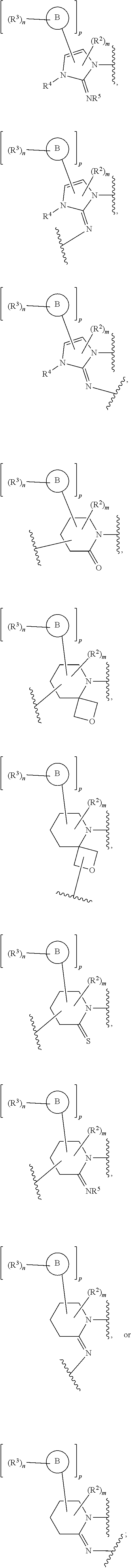

Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, 1'SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
   L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—;
   m is 0, 1, or 2;
   n is 0, 1, 2, 3, or 4;
   p is 0 or 1; and
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
      two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, a compound of formula I-e-1 above is provided as a compound of formula I-e-1′ or formula I-e-1″:

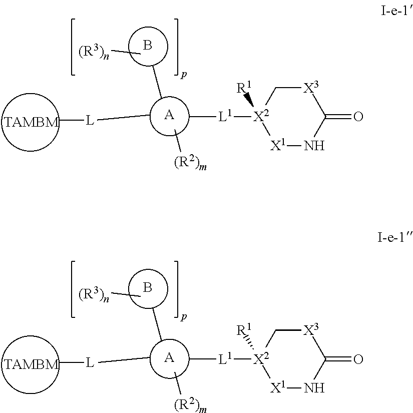

or a pharmaceutically acceptable salt thereof, wherein:
each of TAMBM, Ring A, Ring B, L, L¹, R¹, R², R³, X³, n, p, and m is as defined above.

In certain embodiments, the present invention provides a compound of Formula I, wherein LBM is a VHL E3 ubiquitin ligase binding moiety

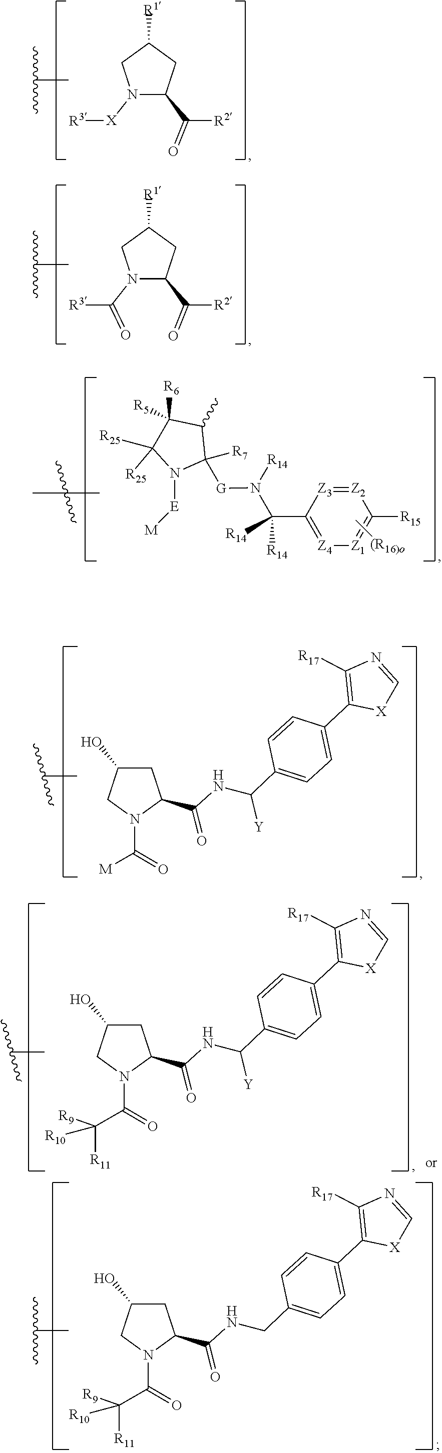

thereby forming a compound of Formulae I-f-1, I-f-2, I-f-3, I-f-4, I-f-5 or I-f-6 respectively:

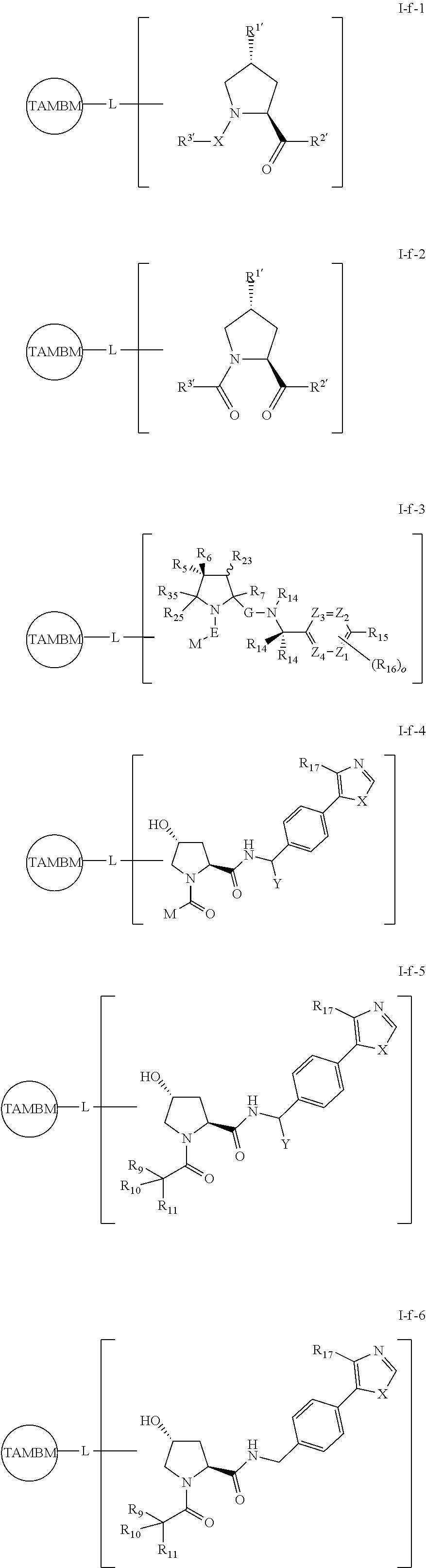

or a pharmaceutically acceptable salt thereof, wherein L and TAMBM are as defined above and described in embodiments herein, and wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 which is herein incorporated by reference in its entirety.

As used herein, depiction of brackets around any LBM

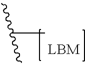

means that the

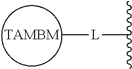

moiety is covalently attached to said LBM at any available modifiable carbon, nitrogen, oxygen, or sulfur atom. For purposes of clarity and by way of example, such available modifiable carbon, nitrogen, oxygen, or sulfur atoms in the following LBM compound structure are depicted below, wherein each wavy bond defines the point of attachment to said

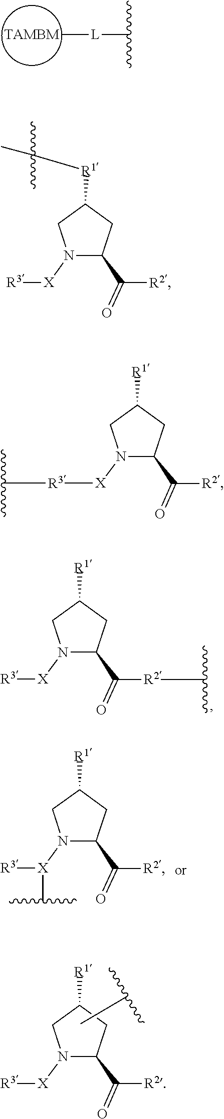

In certain embodiments, the present invention provides a compound of Formula I, wherein LBM is a VHL E3 ubiquitin ligase binding moiety

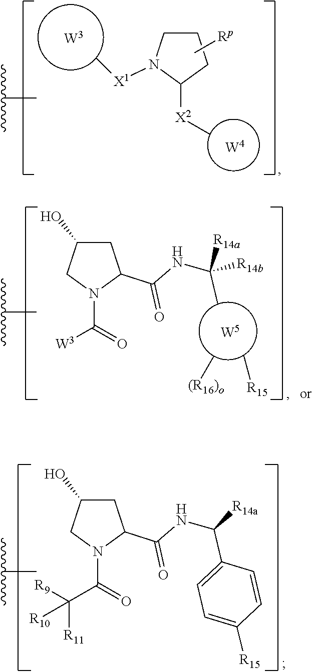

thereby forming a compound of Formulae I-g-1, I-g-2, or I-g-3, respectively:

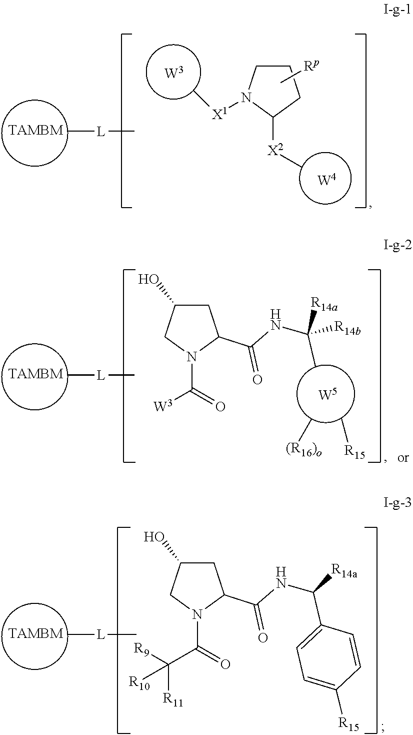

or a pharmaceutically acceptable salt thereof, wherein L and TAMBM are as defined above and described in embodiments herein, and wherein each of the variables R^{p}, R₉, R₁₀, R₁₁, R₁₄ₐ, R_{14b}, R₁₅, R₁₆, W³, W⁴, W⁵, X¹, X², and o is as defined and described in WO 2016/118666 which is herein incorporated by reference in its entirety.

In certain embodiments, the present invention provides a compound of Formula I, wherein LBM is an E3 ubiquitin ligase (cereblon) binding moiety

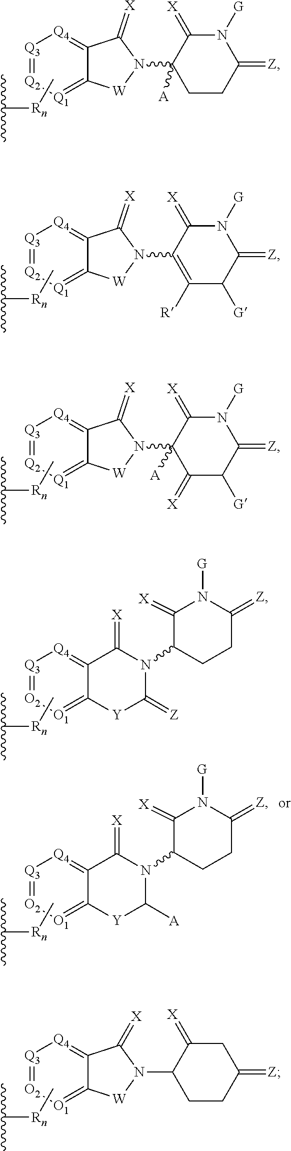

thereby forming a compound of Formulae I-h-1, I-h-2, I-h-3, I-h-4, I-h-5, or I-h-6, respectively:

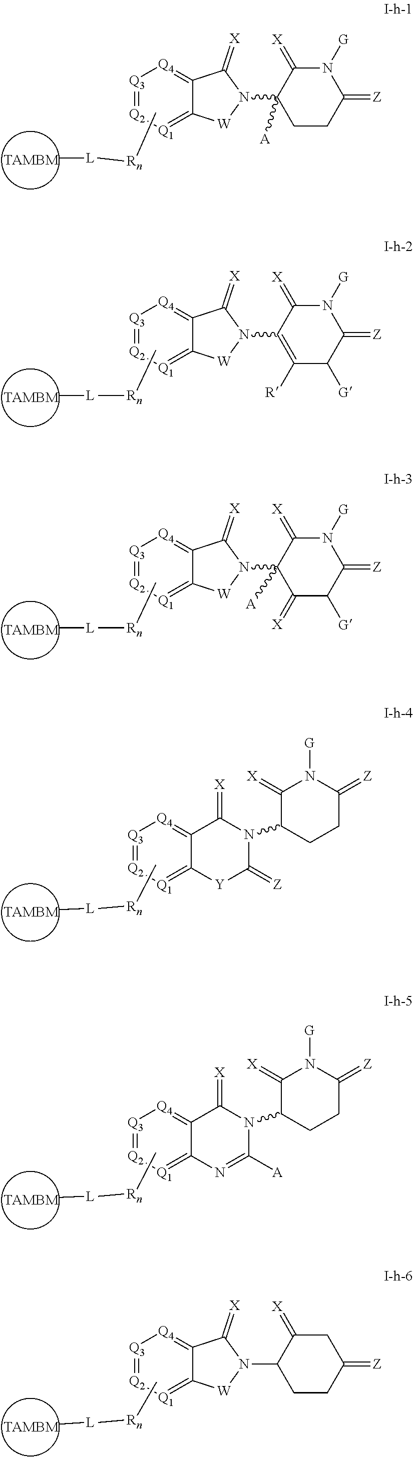

or a pharmaceutically acceptable salt thereof, wherein L and TAMBM are as defined above and described in embodiments herein, and wherein each of the variables A, G, G′, Q₁, Q₂, Q₃, Q₄, R, R′, W, X, Z, and n is as defined and described in WO 2016/197114 and US2018/0147202, which is herein incorporated by reference in its entirety.

In certain embodiments, the present invention provides a compound of Formula I, wherein LBM is a MDM2 (i.e., human double minute 2 or HDM2) E3 ligase binding moiety

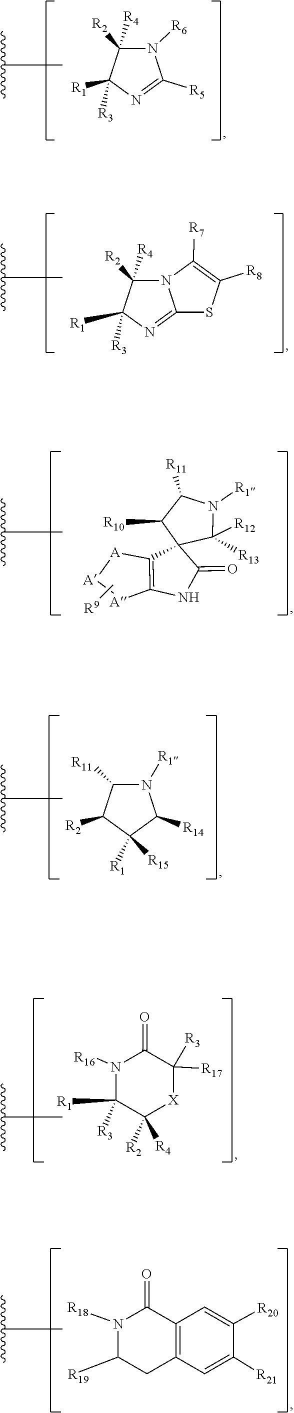

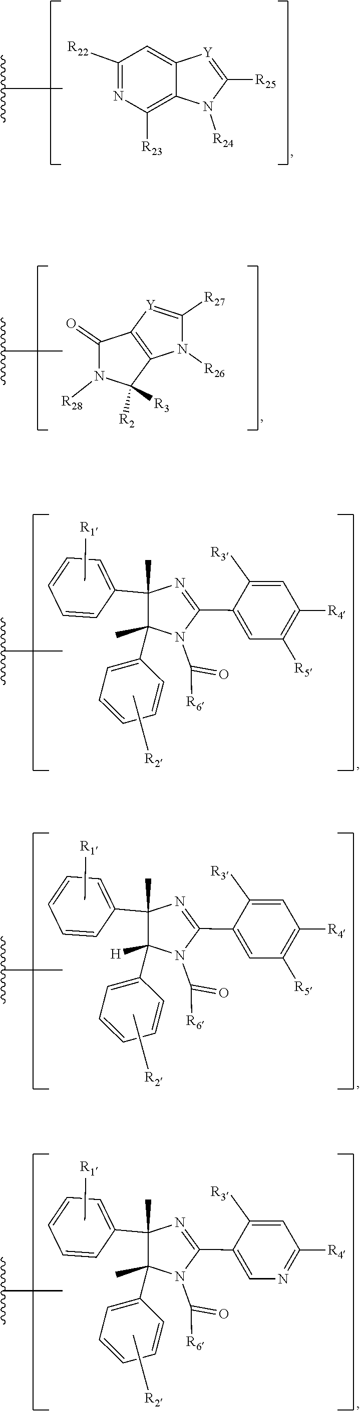

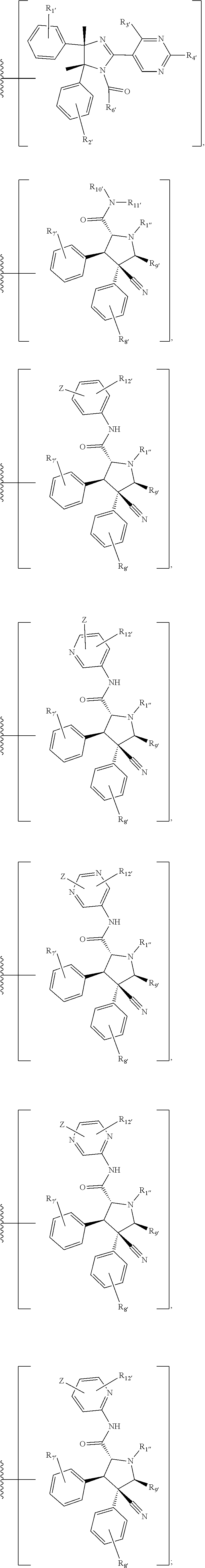

thereby forming a compound of Formulae I-i-1, I-i-2, I-i-3, I-i-4, I-i-5, I-i-6, I-i-7, I-i-8, I-i-9, I-i-10, I-i-11, I-i-12, I-i-13, I-i-14, I-i-15, I-i-16, I-i-17, or I-i-18, respectively:

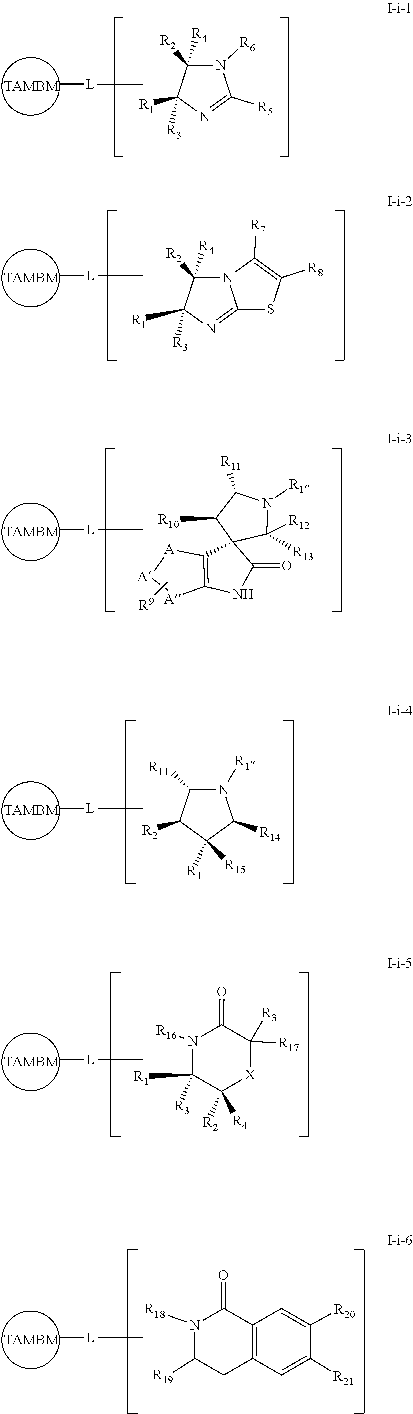

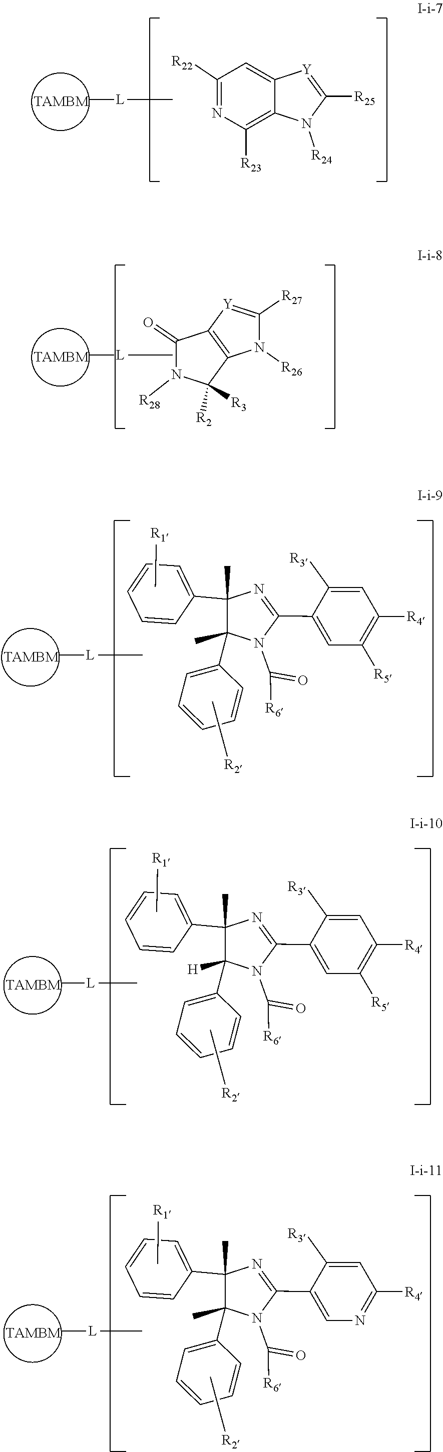

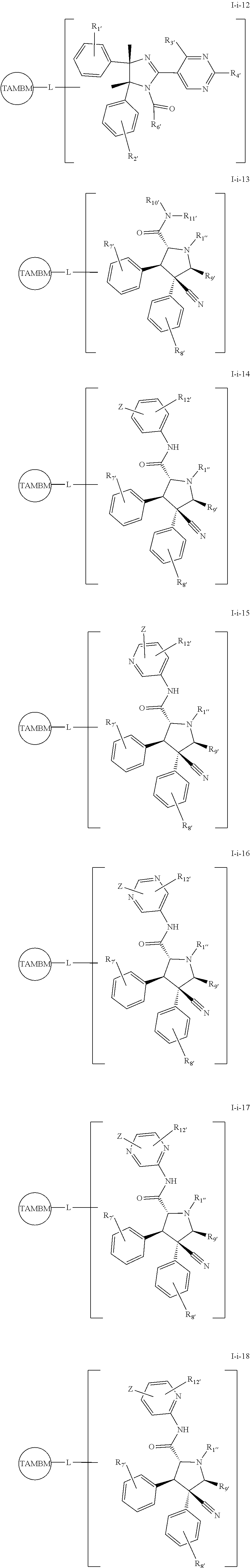

or a pharmaceutically acceptable salt thereof, wherein L and TAMBM are as defined above and described in embodiments herein, and wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{1′}, R_{2′}, R_{3′}, R_{4′}, R_{5′}, R_{6′}, R_{7′}, R_{8′}, R_{9′}, R_{10′}, R_{11′}, R_{12′}, R_{1″}, A, A′, A″, X, Y, and Z is as defined and described in WO 2017/011371 and US 2017/0008904, which is herein incorporated by reference in its entirety.

In certain embodiments, the present invention provides a compound of Formula I, wherein LBM is a CRBN or VHL E3 ubiquitin ligase binding moiety selected from the group consisting of

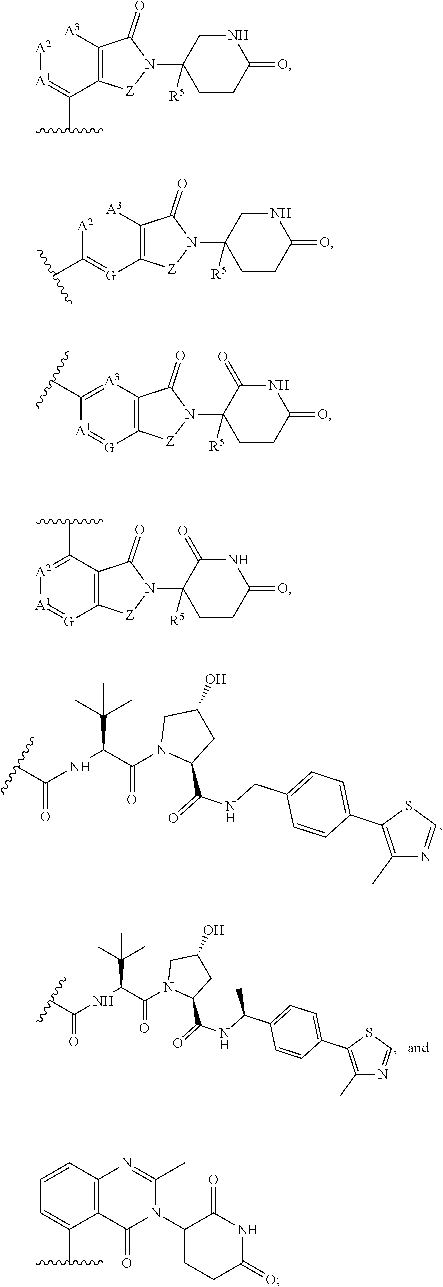

thereby forming a compound of Formulae I-j-1, I-j-2, I-j-3, I-j-4, I-j-5, I-j-6, or I-j-7, respectively:

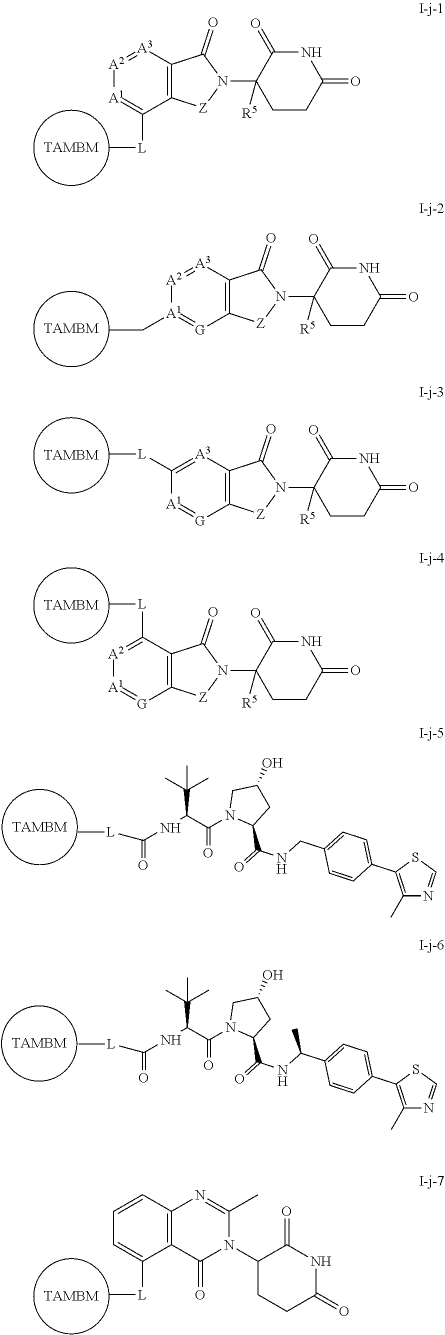

or a pharmaceutically acceptable salt thereof, wherein L and TAMBM are as defined above and described in embodiments herein, and wherein each of the variables A¹, A², A³, R⁵, G and Z is as defined and described in WO 2017/176958 and US2019/0119289, which is herein incorporated by reference in its entirety.

In certain embodiments, the present invention provides a compound of Formula I, wherein LBM is an TAP E3 ubiquitin ligase binding moiety

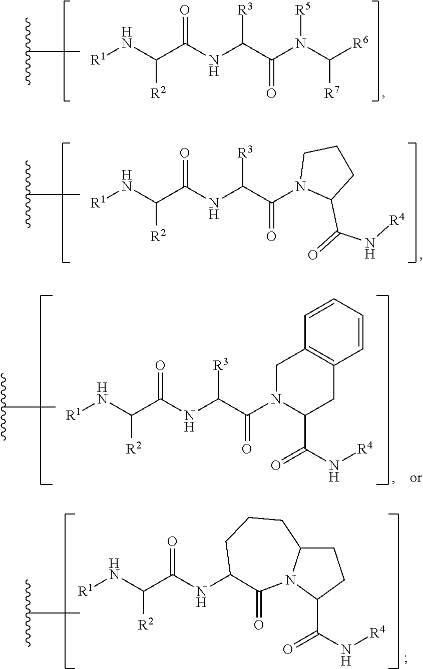

thereby forming a compound of Formula I-k-1, I-k-2, I-k-3, or I-k-4, respectively:

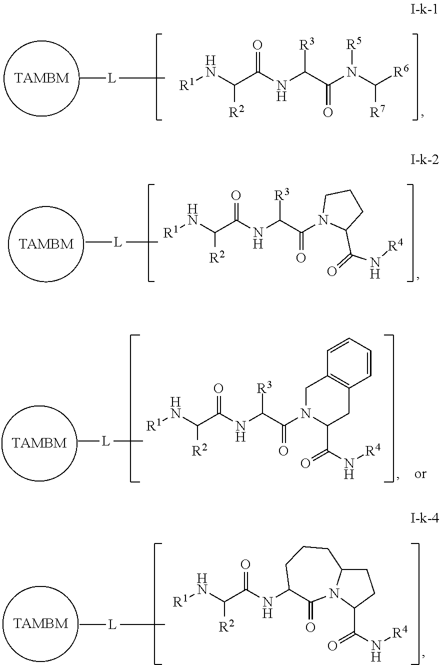

or a pharmaceutically acceptable salt thereof, wherein L and TAMBM are as defined above and described in embodiments herein, and wherein each of the variables R¹, R², R³, R⁴, R⁵, R⁶, and R⁷, is as defined and described in WO 2017/011590 and US 2017/0037004, which is herein incorporated by reference in its entirety.

In certain embodiments, the present invention provides a compound of Formula I, wherein LBM is an E3 ubiquitin ligase (cereblon) binding moiety

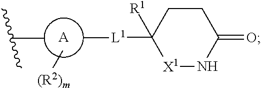

thereby forming a compound of Formula I-1:

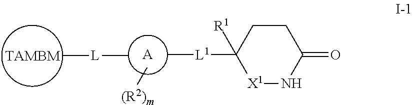

or a pharmaceutically acceptable salt thereof, wherein L and TAMBM are as defined above and described herein, and wherein:
   X¹ is a bivalent moiety selected from a covalent bond, —CH₂—, —C(O)—, —C(S)—, or

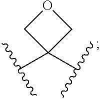 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring A is a bi- or tricyclic ring selected from

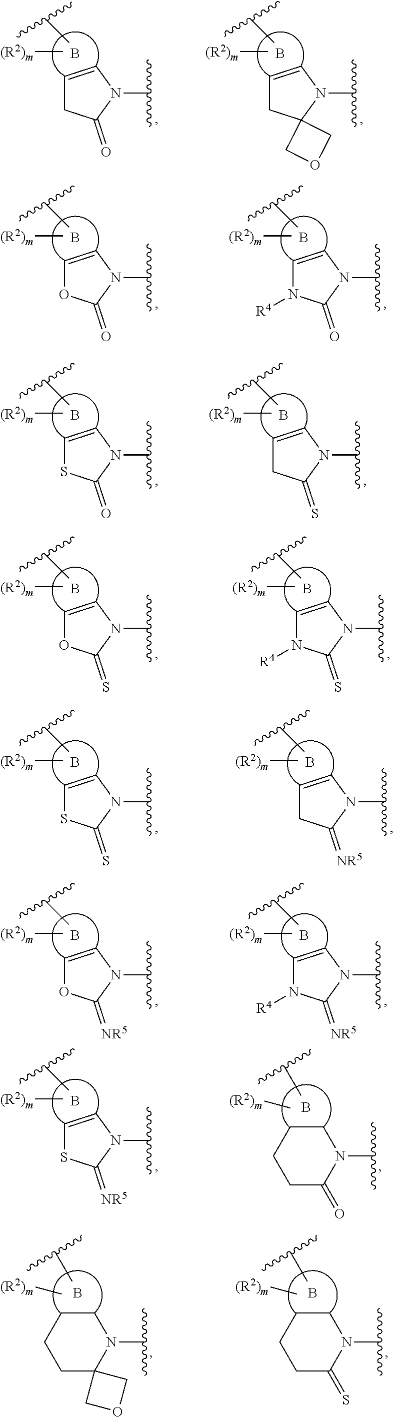

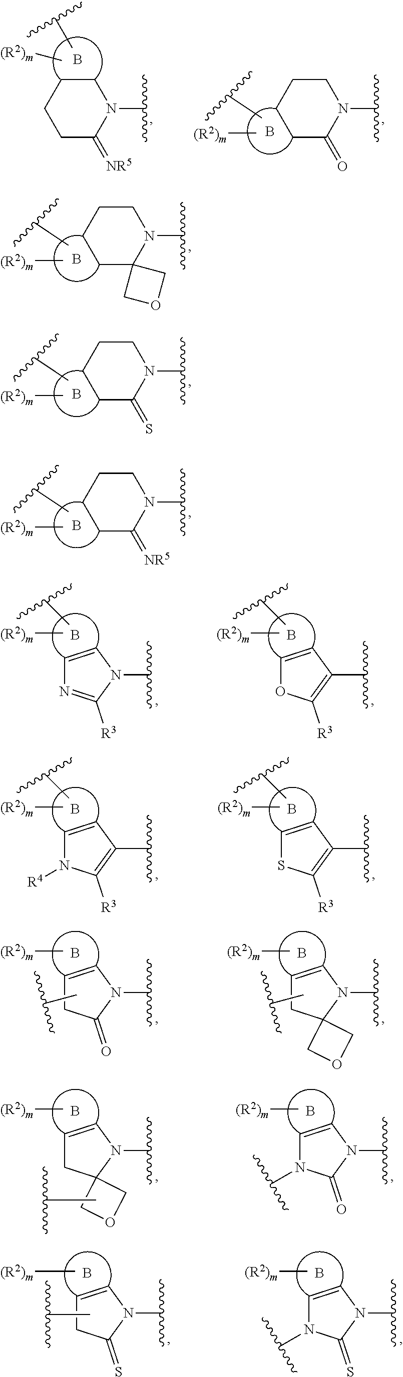

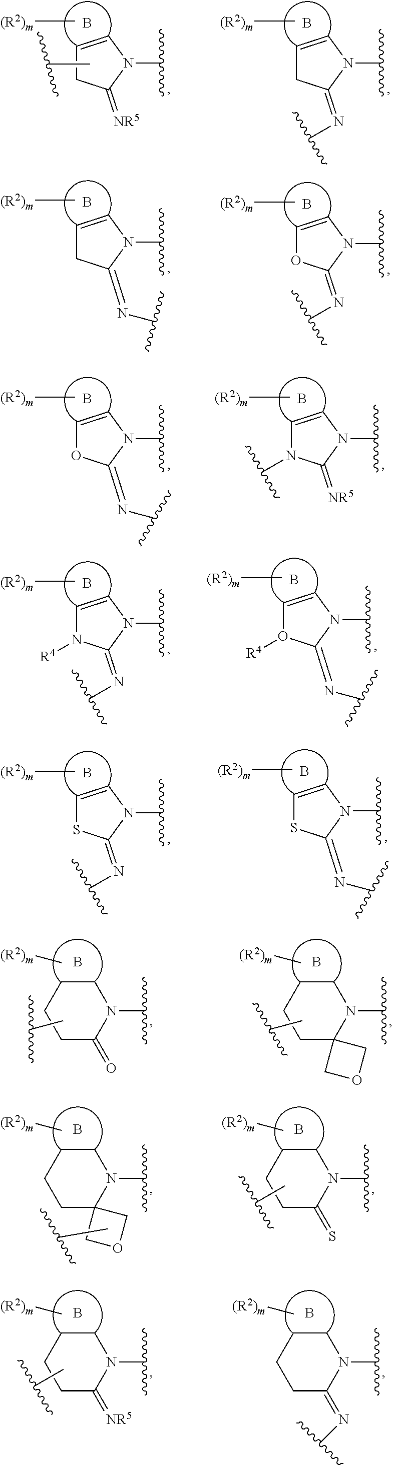

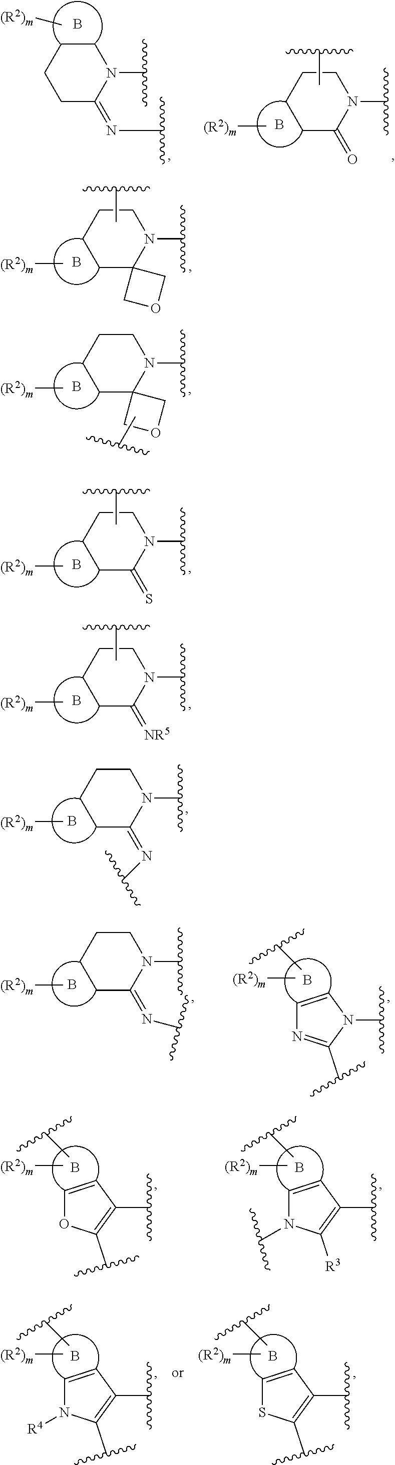

wherein:
Ring B is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

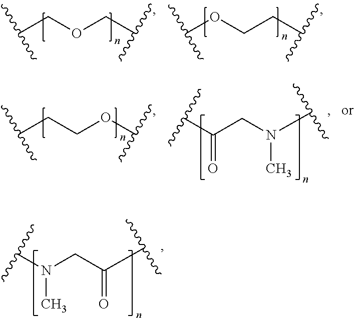

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
m is 0, 1, 2, 3 or 4;
each of n is independently 1, 2, 3, 4, 5, 6, 7, 8, 9, or 10; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

Where a point of attachment of —(R²)ₙ is depicted on Ring B, it is intended, and one of ordinary skill in the art would appreciate, that the point of attachment of —(R²)ₙ may be on Ring A and may also be at any available carbon or nitrogen atom on Ring A including the ring to which Ring B is fused. Where —R² is attached to a nitrogen atom bound to R⁴ or R⁵, R⁴ or R⁵ is absent and —R² takes the place of the R⁴ or R⁵ group. Where —R² is attached to a carbon atom bound to R³, R³ is absent and —R² takes the place of the R³ group.

In certain embodiments, the present invention provides a compound of Formula I, wherein LBM is an E3 ubiquitin ligase (cereblon) binding moiety

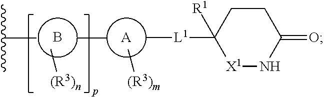

thereby forming a compound of Formula I-m:

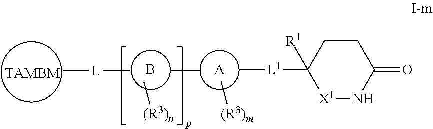

or a pharmaceutically acceptable salt thereof, wherein, L and TAMBM are as defined above and described in embodiments herein, and wherein:
   X¹ is a bivalent moiety selected from a covalent bond, —CH₂—, —C(O)—, —C(S)—, or

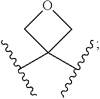 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring A is a mono- or bicyclic ring selected from

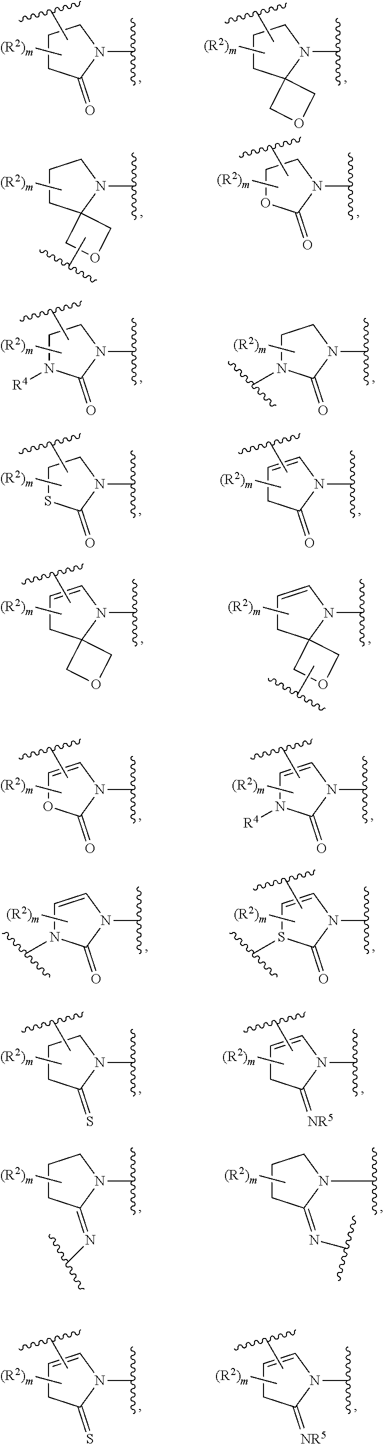

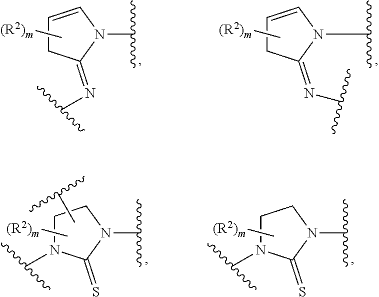

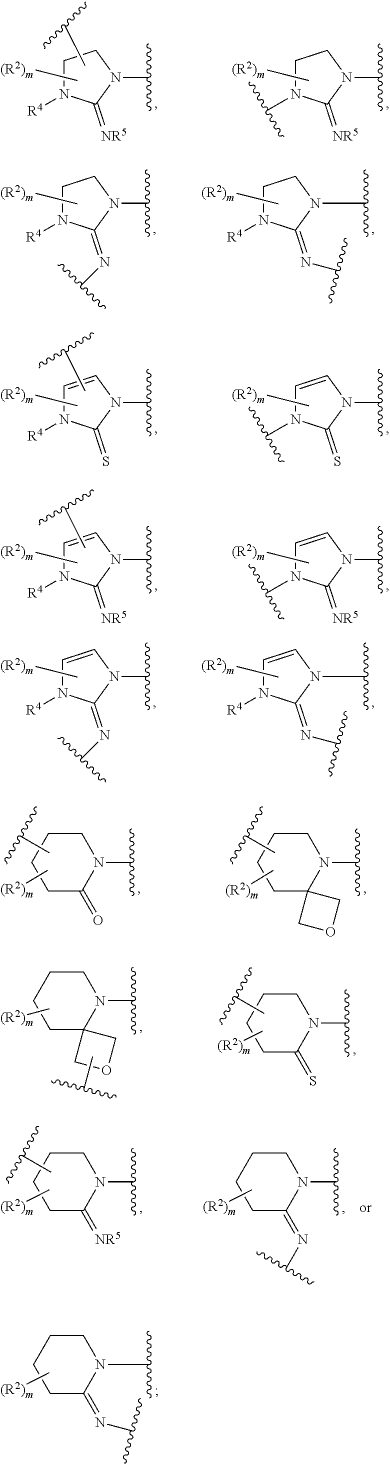

each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
—N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
—N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

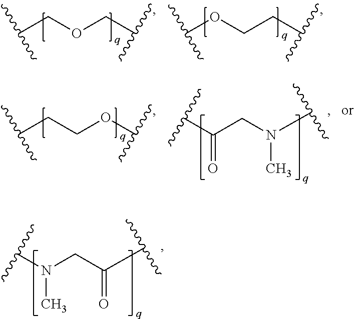

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
m is 0, 1, or 2;
n is 0, 1, 2, 3, or 4;
p is 0 or 1, wherein when p is 0, the bond connecting Ring A and Ring B is connected to

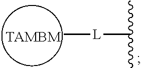

each of q is independently 1, 2, 3, 4, 5, 6, 7, 8, 9, or 10; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety

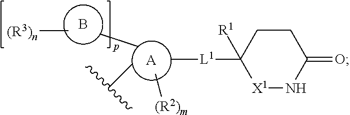

thereby forming a compound of Formula I-n:

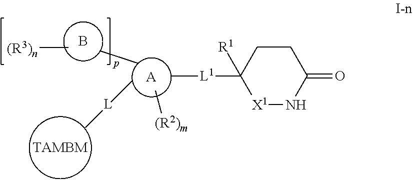

or a pharmaceutically acceptable salt thereof, wherein L and TAMBM are as defined above and described in embodiments herein, and wherein:
   X¹ is a bivalent moiety selected from a covalent bond, —CH₂-, —C(O)—, —C(S)—, or

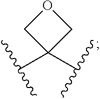 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;

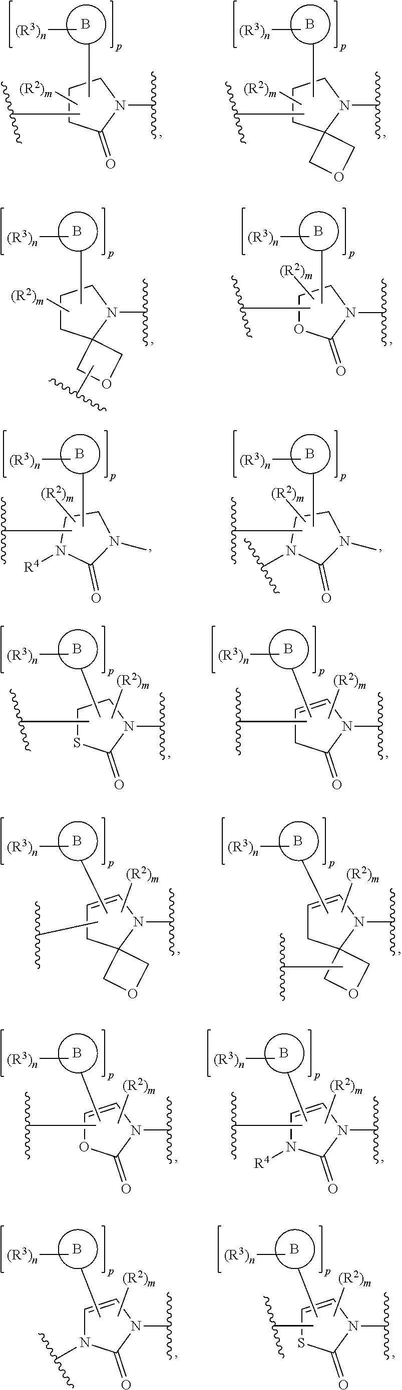

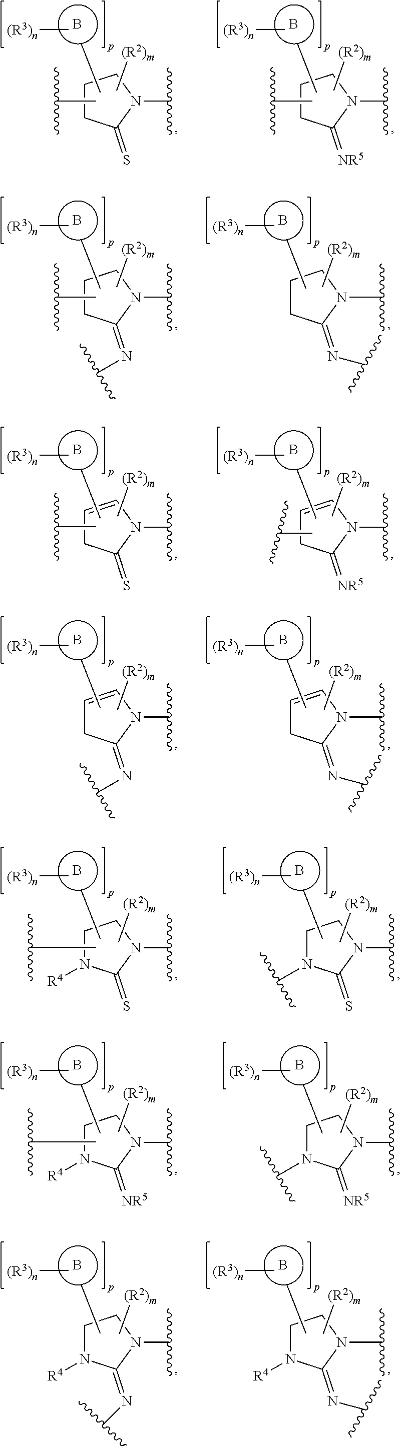

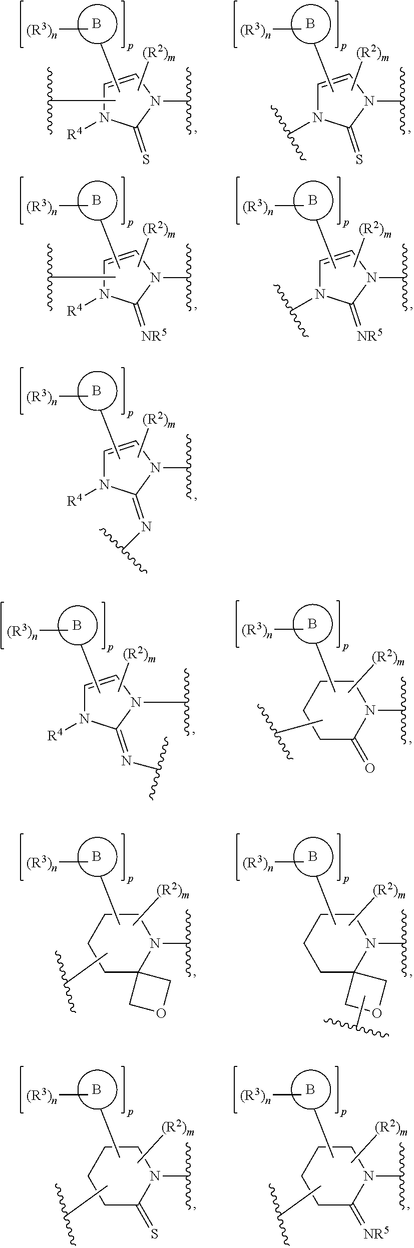

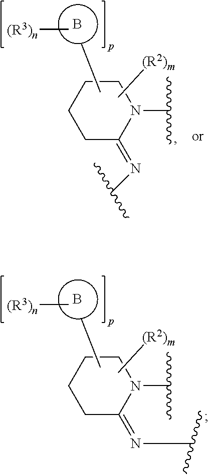

Ring A is a mono- or bicyclic ring selected from
each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

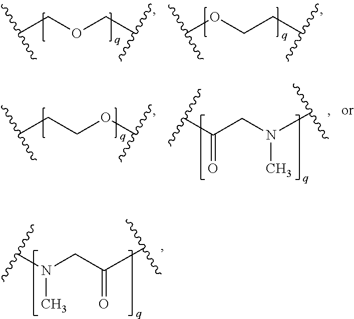

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
m is 0, 1, or 2;
n is 0, 1, 2, 3, or 4;
p is 0 or 1;
each of q is independently 1, 2, 3, 4, 5, 6, 7, 8, 9, or 10; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety

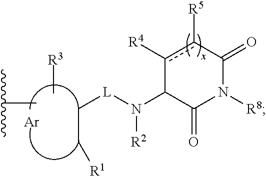

thereby forming a compound of Formula I-o:

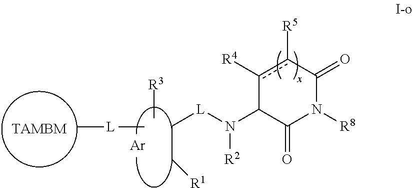

or a pharmaceutically acceptable salt thereof, wherein L and TAMBM are as defined above and described in embodiments herein, and wherein each of the variables Ar, R¹, R², R³, R⁴, R⁵, R⁸, L, x, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

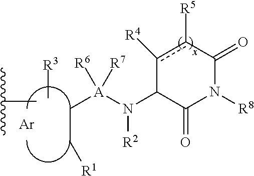

thereby forming a compound of Formula I-p:

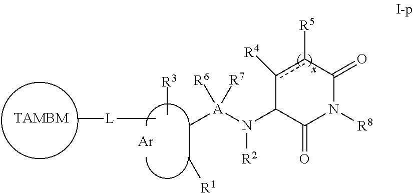

or a pharmaceutically acceptable salt thereof, wherein L and TAMBM are as defined above and described in embodiments herein, and wherein each of the variables Ar, R¹, R², R³, R⁴, R⁵, R⁶, R⁷, R⁸, A, x, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

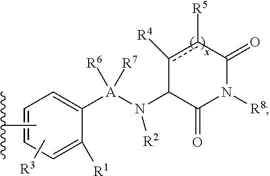

thereby forming a compound of Formula I-q:

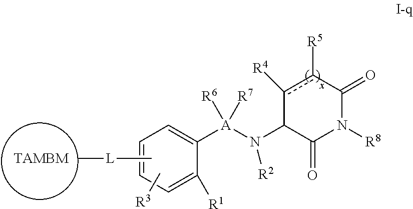

or a pharmaceutically acceptable salt thereof, wherein L and TAMBM are as defined above and described in embodiments herein, and wherein each of the variables R¹, R², R³, R⁴, R⁵, R⁶, R⁷, R⁸, A, x, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

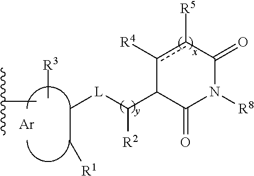

thereby forming a compound of Formula I-r:

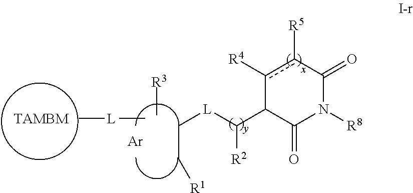

or a pharmaceutically acceptable salt thereof, wherein L and TAMBM are as defined above and described in embodiments herein, and wherein each of the variables Ar, R¹, R², R³, R⁴, R⁵, R⁸, L, x, y, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

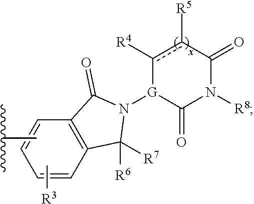

thereby forming a compound of Formula I-s:

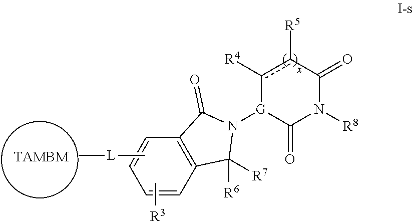

or a pharmaceutically acceptable salt thereof, wherein L and TAMBM are as defined above and described in embodiments herein, and wherein each of the variables G, R³, R⁴, R⁵, R⁶, R⁷, R⁸, x, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

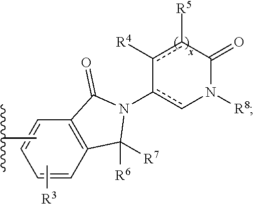

thereby forming a compound of Formula I-t:

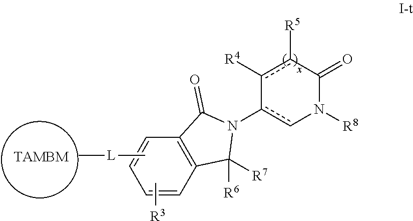

or a pharmaceutically acceptable salt thereof, wherein L and TAMBM are as defined above and described in embodiments herein, and wherein each of the variables R³3, R⁴, R⁵, R⁶R⁷, R⁸, x, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

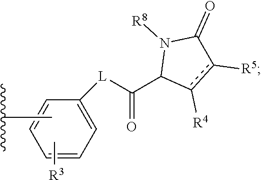

thereby forming a compound of Formula I-u:

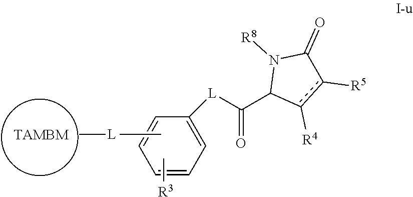

or a pharmaceutically acceptable salt thereof, wherein L and TAMBM are as defined above and described in embodiments herein, and wherein each of the variables R³, R⁴, R⁵, R⁸, L, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

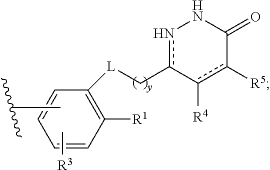

thereby forming a compound of Formula I-v:

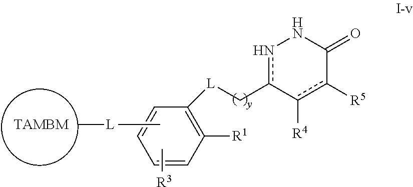

or a pharmaceutically acceptable salt thereof, wherein L and TAMBM are as defined above and described in embodiments herein, and wherein each of the variables R′, R³, R⁴, R⁵, L, y, and the bond [symbol] is as described and defined in WO 2017/161119, which is herein incorporated by reference in its entirety.

In certain embodiments, the present invention provides a compound of Formula I, wherein LBM is an E3 ubiquitin ligase (cereblon) binding moiety

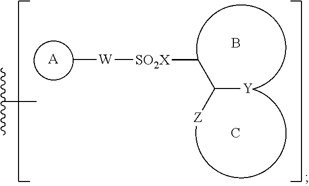

thereby forming a compound of Formula I-x:

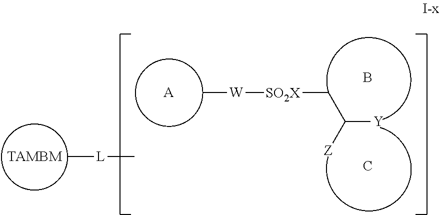

or a pharmaceutically acceptable salt thereof, wherein L and TAMBM are as defined above and described in embodiments herein, and wherein each of the variables A, B, C, W, X, Y, and Z is as described and defined in U.S. Pat. No. 5,721,246, which is herein incorporated by reference in its entirety.

In certain embodiments, the present invention provides a compound of Formula I, wherein LBM is an E3 ubiquitin ligase (cereblon) binding moiety a

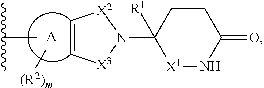

DCAF15 E3 ubiquitin ligase binding moiety

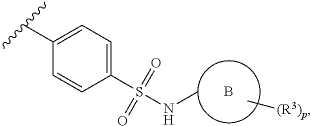

or a VHL E3 ubiquitin ligase binding moiety

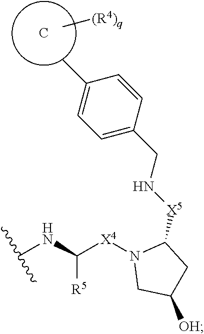

thereby forming a compound of Formulae I-y-1, I-y-₂, or I-y-3:

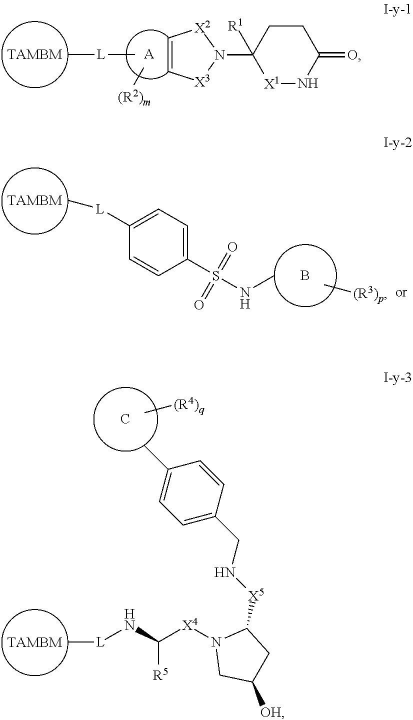

or a pharmaceutically acceptable salt thereof, wherein TAMBM is as defined above and described in embodiments herein, and wherein:
   each of X¹, X², and X³ is independently a bivalent moiety selected from a covalent bond, —CH₂—, —C(O)—, —C(S)—, or

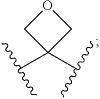 [SMILES: *C1(*)COC1]

each of X⁴ and X⁵ is independently a bivalent moiety selected from —CH₂—, —C(O)—, —C(S)—, or

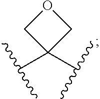 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R³, and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
   —N(R)S(O)₂R;
R⁵ is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B is selected from 6-membered aryl containing 0-2 nitrogen atoms or a 8-10 membered bicyclic heteroaryl having 1-5 heteroatoms independently selected from nitrogen, oxygen, or sulfur;
Ring C is a selected from 6-membered aryl containing 0-2 nitrogen atoms or a 5-membered heteroaryl with 1-3 heteroatoms independently selected from nitrogen, oxygen or sulfur;
L is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

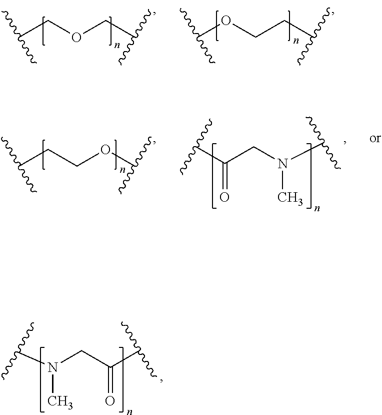

wherein:
each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur;
m is 0, 1, 2, 3 or 4;
each of n is independently 1, 2, 3, 4, 5, 6, 7, 8, 9, or 10;
p is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety

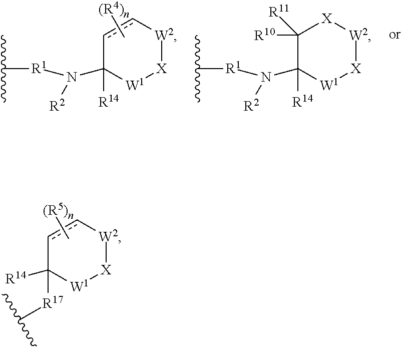

thereby forming a compound of Formulae I-z-1, I-z-₂, or I-z-3, respectively:

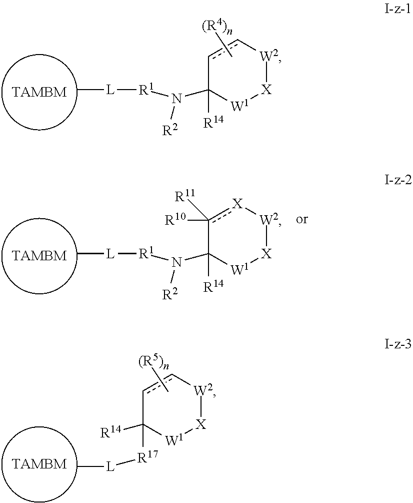

or a pharmaceutically acceptable salt thereof, wherein L and TAMBM are as defined above and described herein, and wherein each of the variables R¹, R², R⁴, R⁵, R¹⁰, R¹¹, R¹⁴, R¹⁷, W¹, W², X and n is as defined in WO 2017/197051 and US 2019/0076539, which is herein incorporated by reference in its entirety and wherein

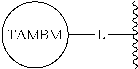

is attached to R¹, the ring formed by combining R¹ and R², or R¹⁷ at the site of attachment of R¹² as defined in WO 2017/197051 such that

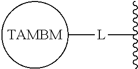

takes the place of the R¹² substituent.

In certain embodiments, the present invention provides a compound of Formula I, wherein LBM is an E3 ubiquitin ligase (cereblon) binding moiety

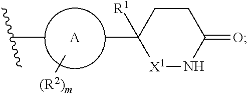

thereby forming a compound of Formula I-aa:

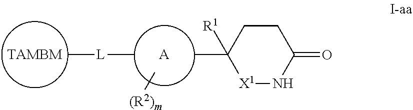

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

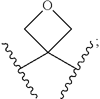 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or
   —N(R)S(O)₂R;
Ring A is a bi- or tricyclic ring selected from wherein

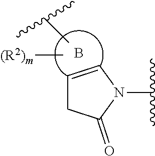

Ring B is other than imidazo or benzo,

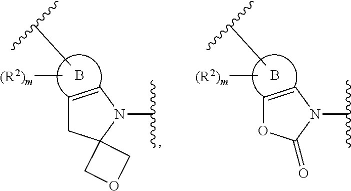

wherein Ring B is other than benzo,

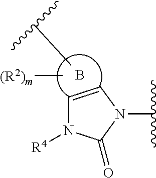

wherein Ring B is other than benzo,

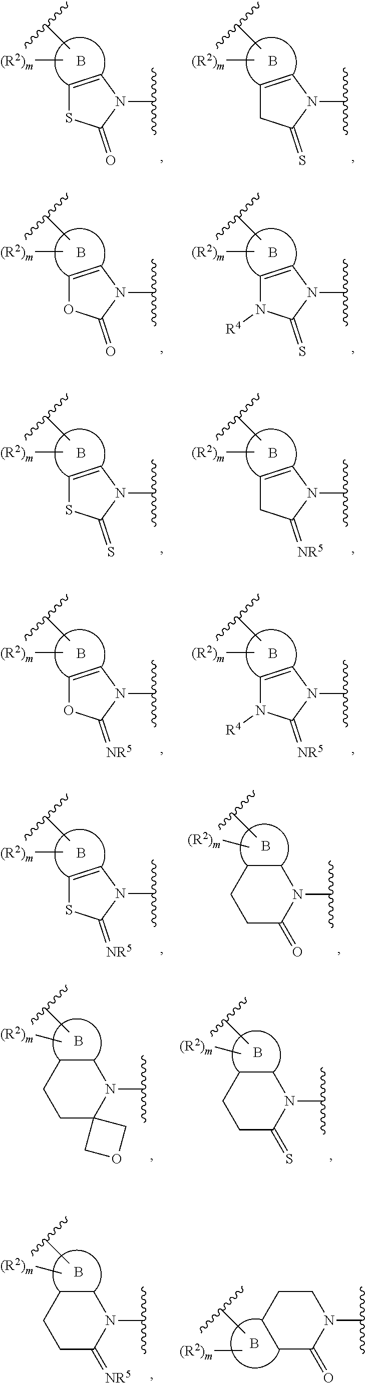

wherein Ring B is other than

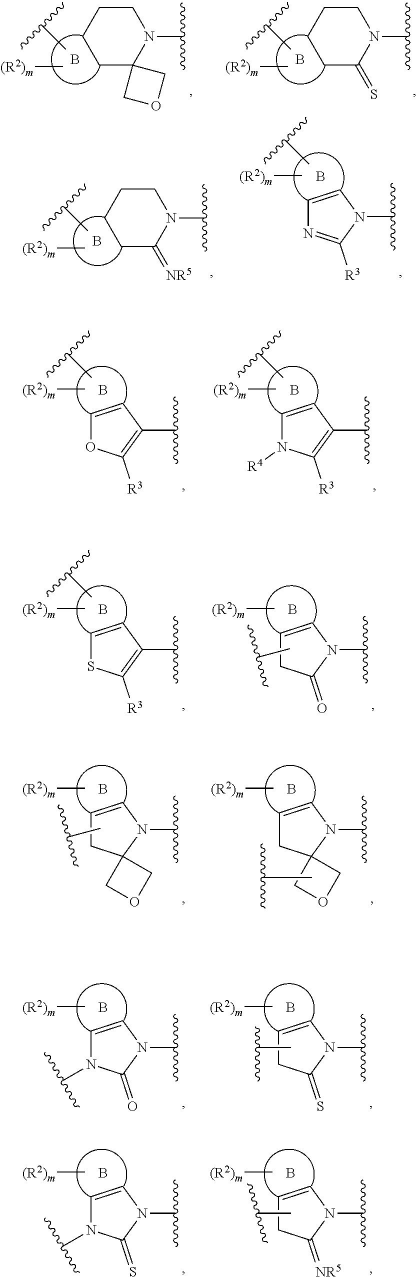

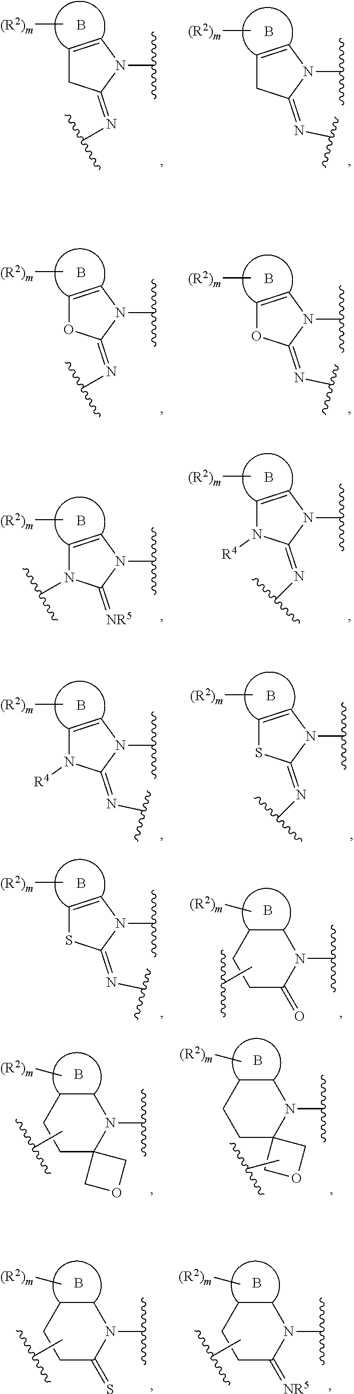

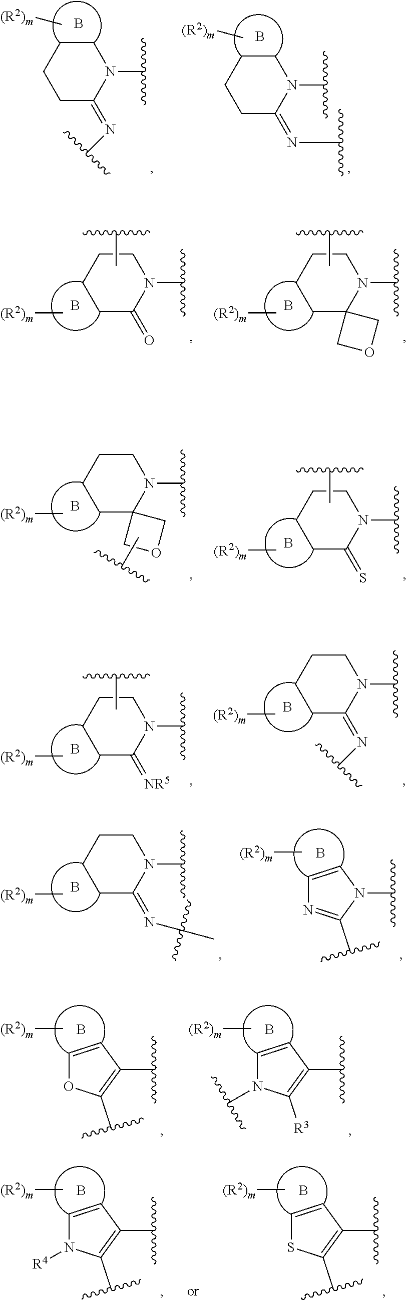

benzo,
wherein:
Ring B is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety

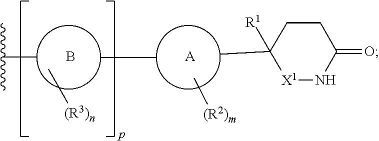

thereby forming a compound of Formula I-bb:

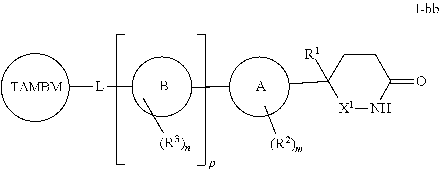

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

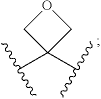 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring A is a mono- or bicyclic ring selected from

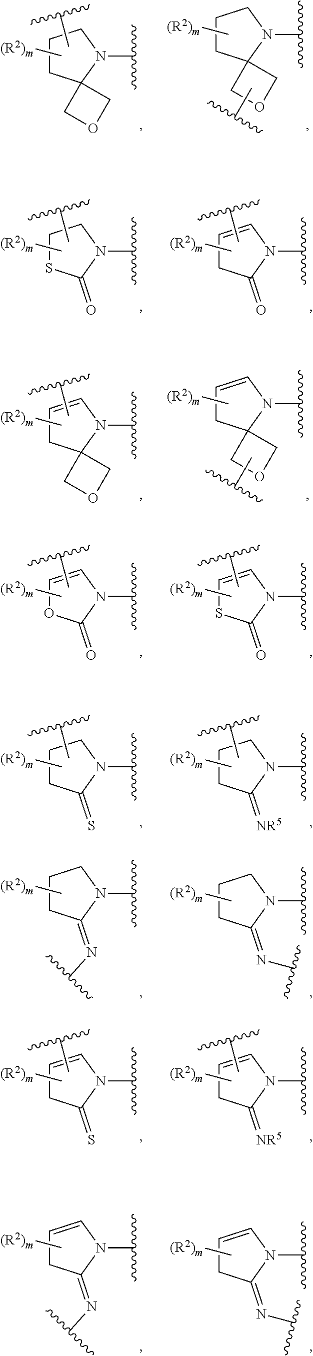

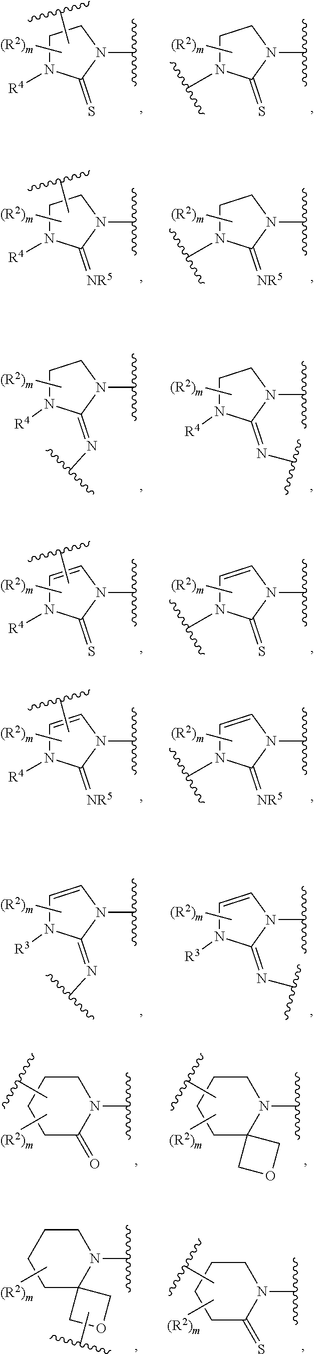

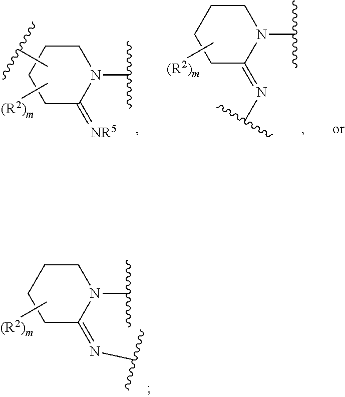

each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1, wherein when p is 0, the bond connecting Ring A and Ring B is connected to

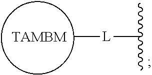

and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In certain embodiments, the present invention provides a compound of Formula I, wherein LBM is an E3 ubiquitin ligase (cereblon) binding moiety

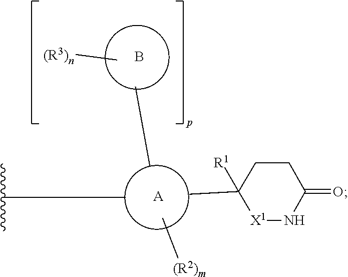

thereby forming a compound of Formula I-cc:

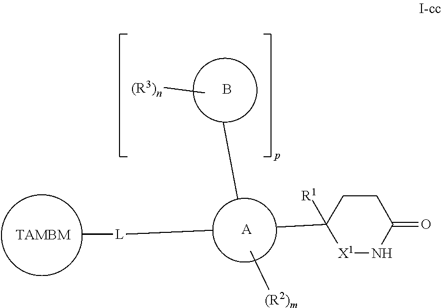

or a pharmaceutically acceptable salt thereof, wherein L and TAMBM are as defined above and described in embodiments herein, and wherein:
   X¹ is a bivalent moiety selected from a covalent bond, —CH₂—, —C(O)—, —C(S)—, or

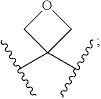 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
Ring A is a mono- or bicyclic ring selected from

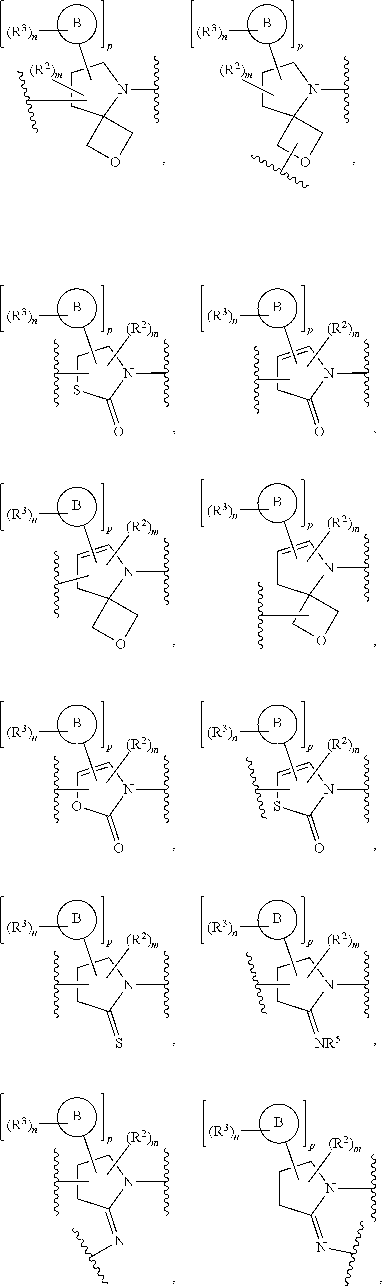

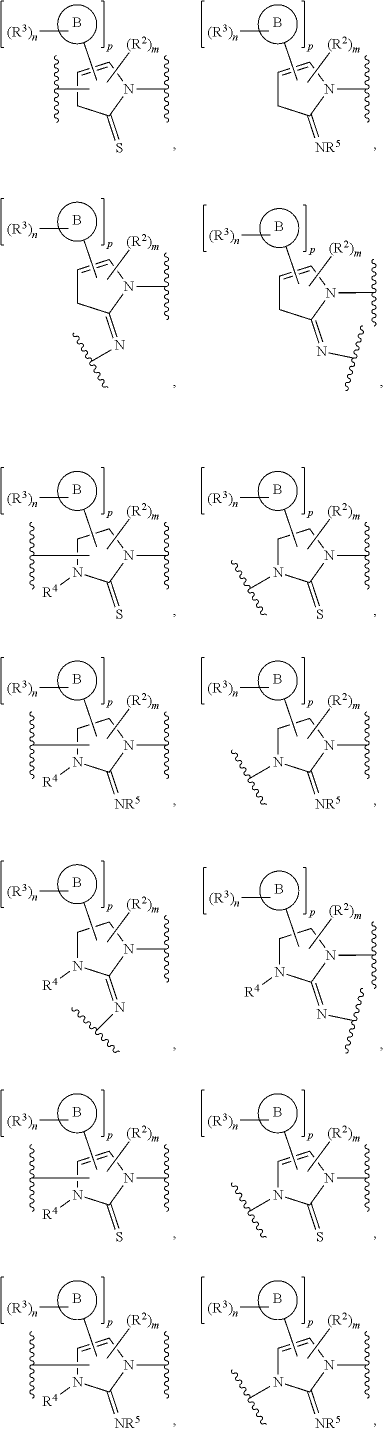

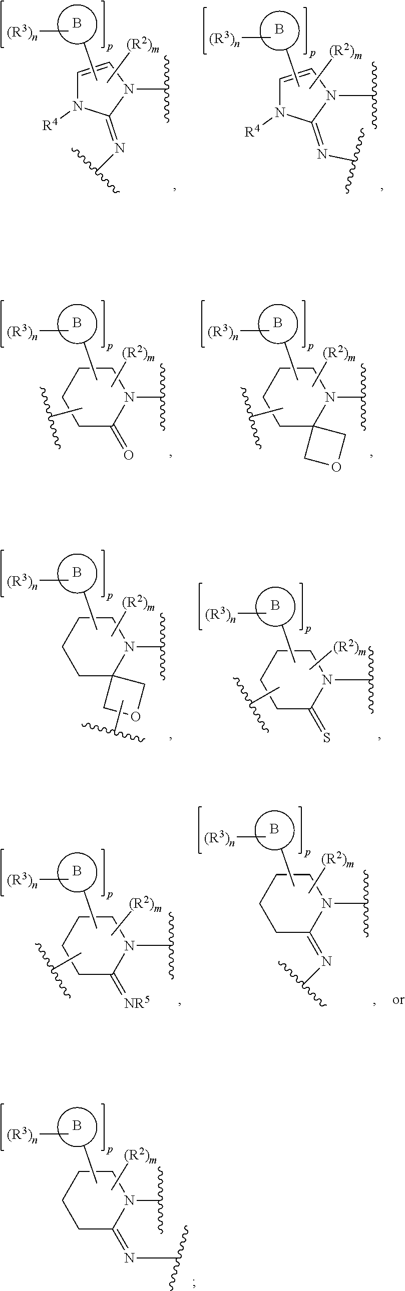

each R² is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
m is 0, 1, or 2;
n is 0, 1, 2, 3, or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

TAMBM

In certain embodiments, the present invention provides a compound of Formula I, wherein TAMBM is a pan-TAM binding moiety, binding Tyro3, Axl, and MerTK. In certain embodiments, TAMBM preferentially binds one or two TAM receptor kinases. In some embodiments, TAMBM preferentially binds Tyro3 and Axl relative to MerTK. In some embodiments, TAMBM preferentially binds Tyro3 and MerTK relative to Axl. In some embodiments, TAMBM preferentially binds MerTK and Axl relative to Tyro3. In some embodiments, TAMBM preferentially binds one TAM receptor kinase. In some embodiments, TAMBM is a Tyro3 binding moiety (TBM). In some embodiments, TAMBM is an Axl binding moiety (ABM). In some embodiments, TAMBM is a MerTK binding moiety (MBM).

In certain embodiments, the present invention provides a compound of Formula I, wherein TAMBM is a TAM receptor kinase inhibitor

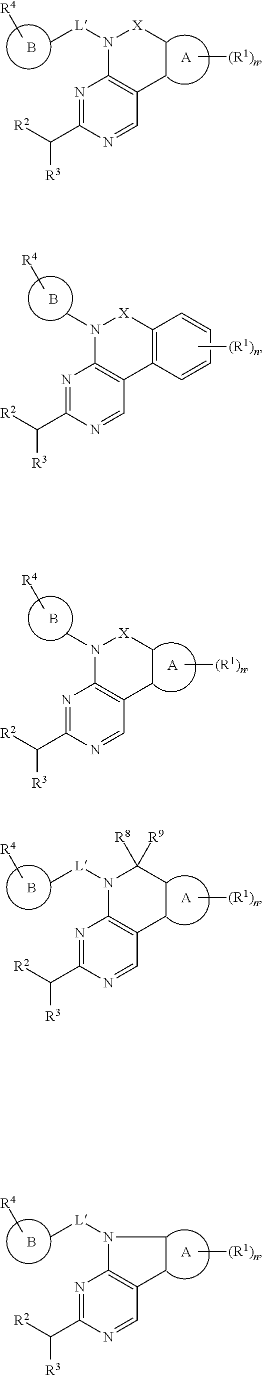

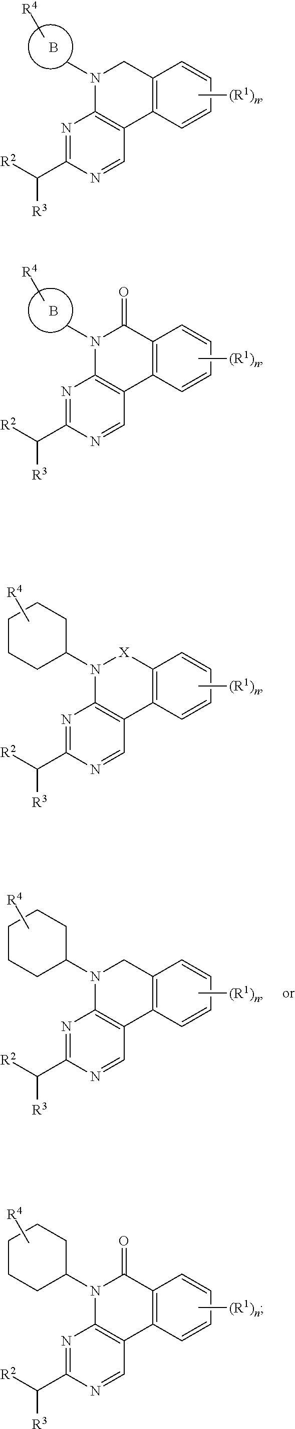

thereby forming a compound of Formulae I-dd-1, I-dd-2, I-dd-3, I-dd-4, I-dd-5, I-dd-6, I-dd-7, I-dd-8, I-dd-9, or I-dd-10, respectively:

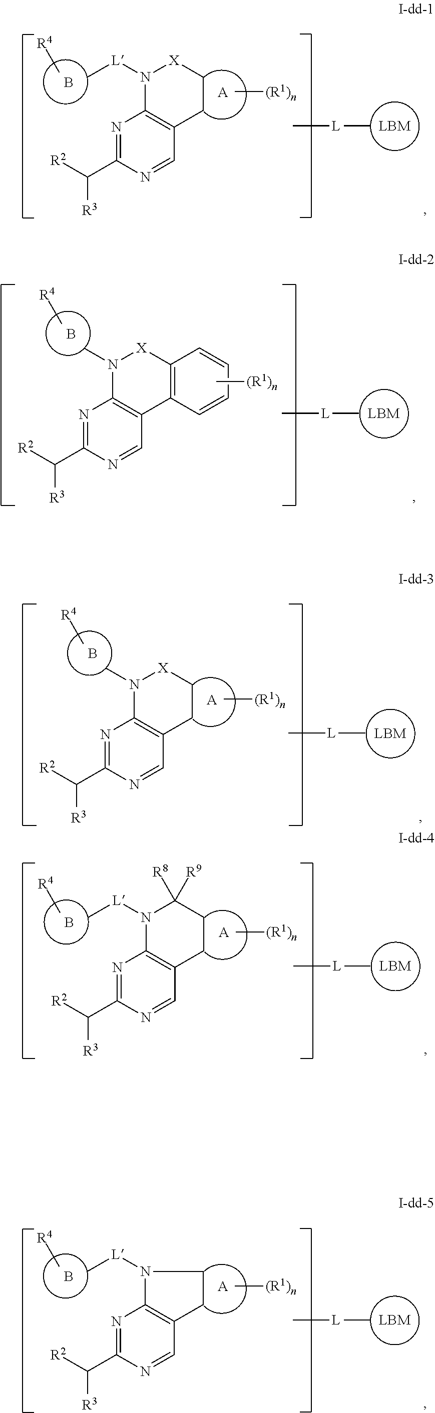

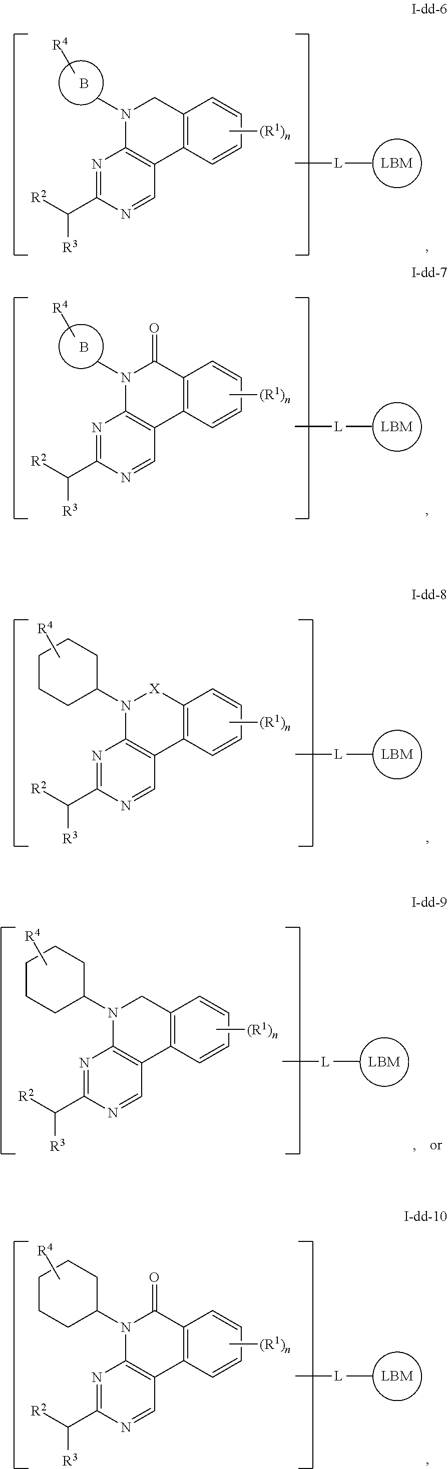

or a pharmaceutically acceptable salt thereof, wherein L and LBM are as defined above and described in embodiments herein, and wherein each of the variables Ring A, Ring B, R¹, R², R³, R⁴, R⁸, R⁹, L′, X, and n are as defined and described in U.S. Pat. No. 9,840,503 and WO 2016/183071, which are incorporated herein, in their entireties.

In certain embodiments, the present invention provides a compound of Formula I, wherein TAMBM is a TAM receptor kinase inhibitor

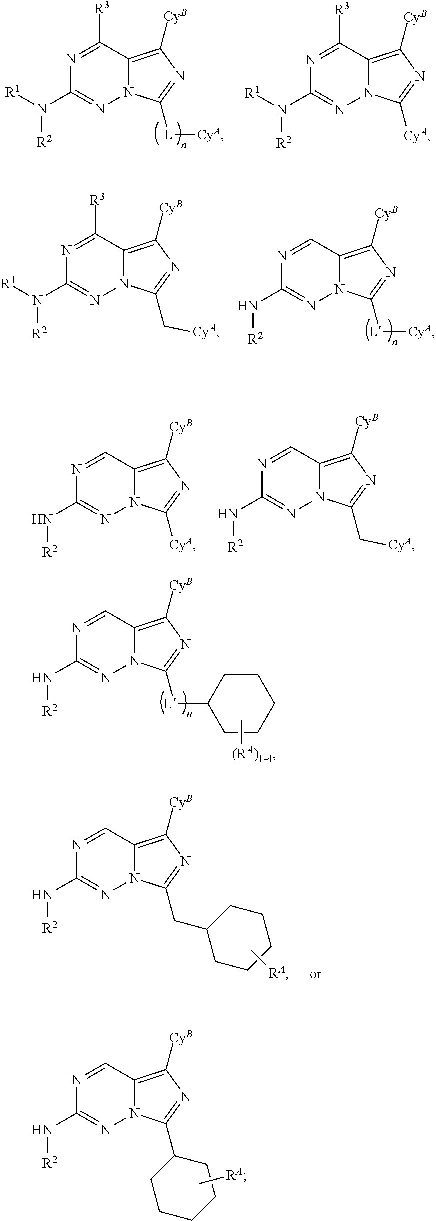

thereby forming a compound of Formulae I-ee-1, I-ee-2, I-ee-3, I-ee-4, I-ee-5, I-ee-6, I-ee-7, I-ee-8, or I-ee-9, respectively:

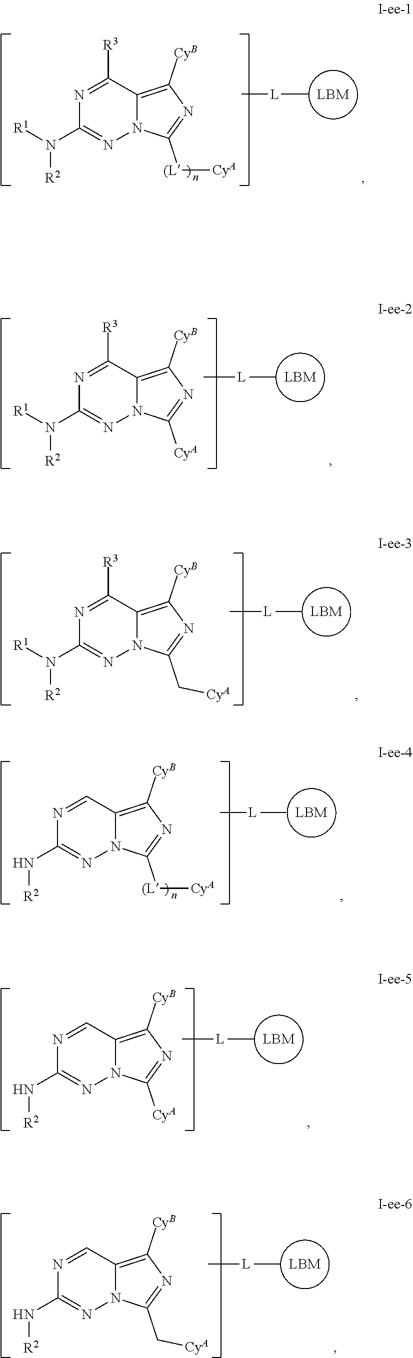

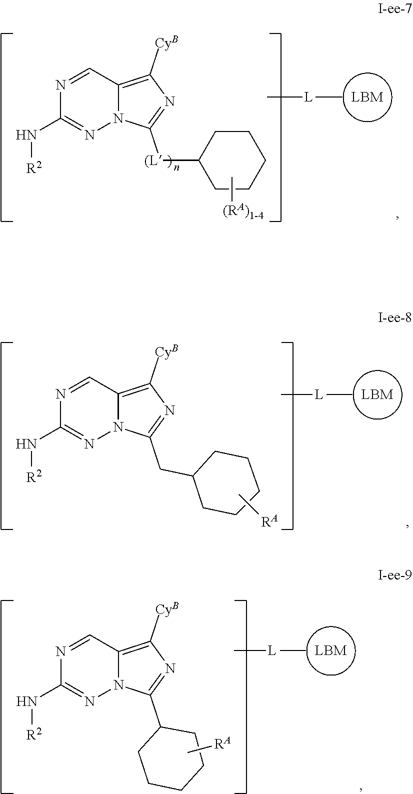

or a pharmaceutically acceptable salt thereof, wherein L and LBM are as defined above and described in embodiments herein, and wherein each of the variables Cy^{A}, Cy^{B}, L′, R¹, R², R³, R^{A}, and n are as defined and described in U.S. Pat. No. 9,708,333, U.S. Pat. App. Pub. No. 2018/0009815, and WO 2017/027717, which are incorporated herein by reference, in their entireties.

In certain embodiments, the present invention provides a compound of Formula I, wherein TAMBM is a TAM receptor kinase inhibitor

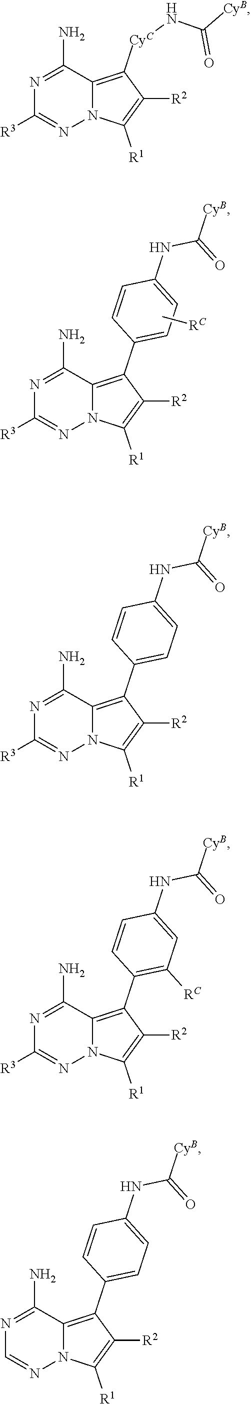

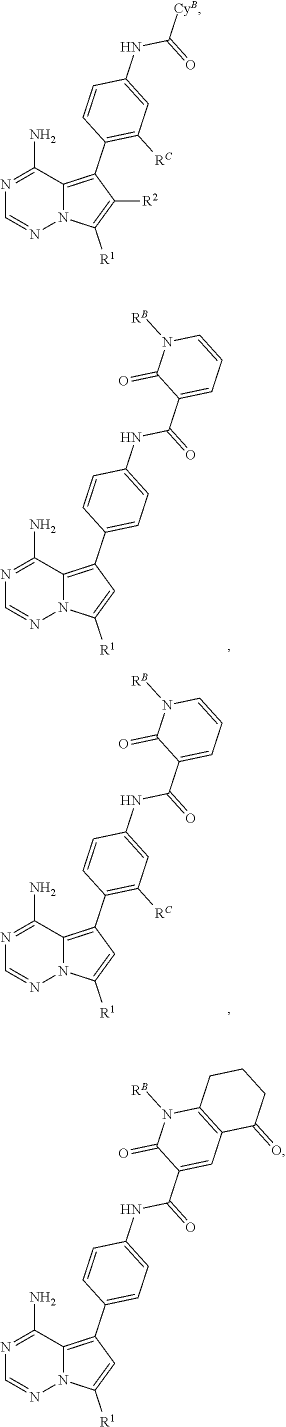

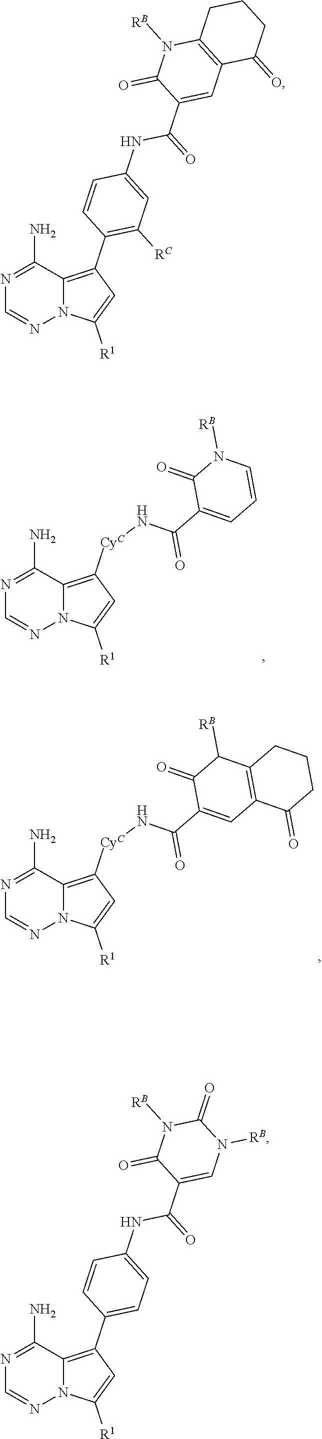

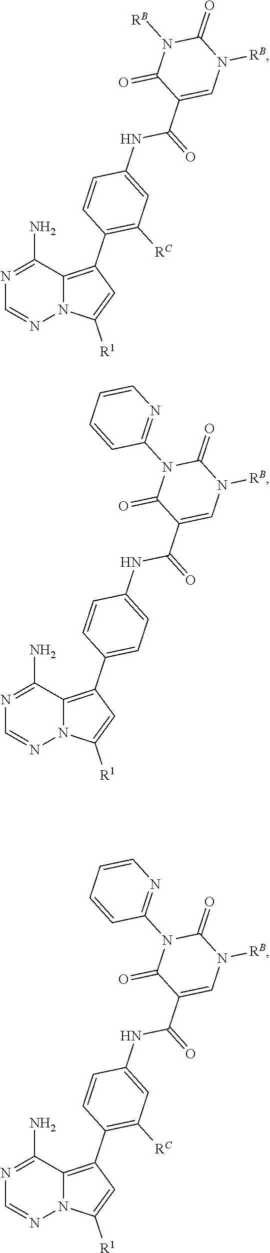

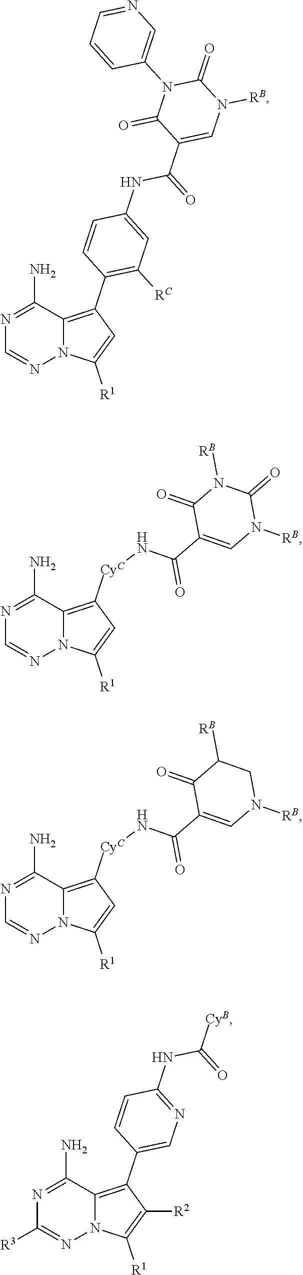

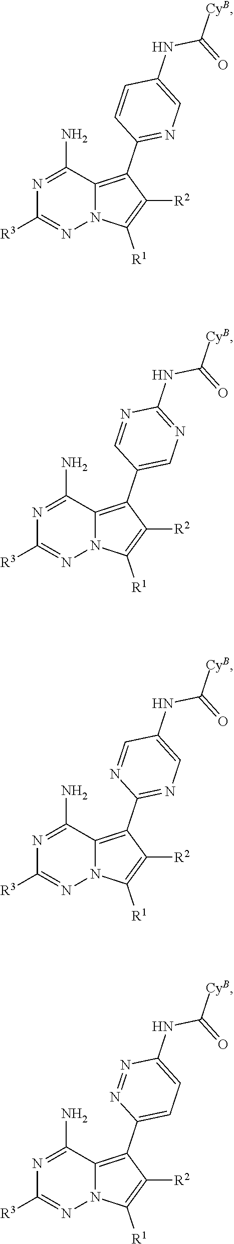

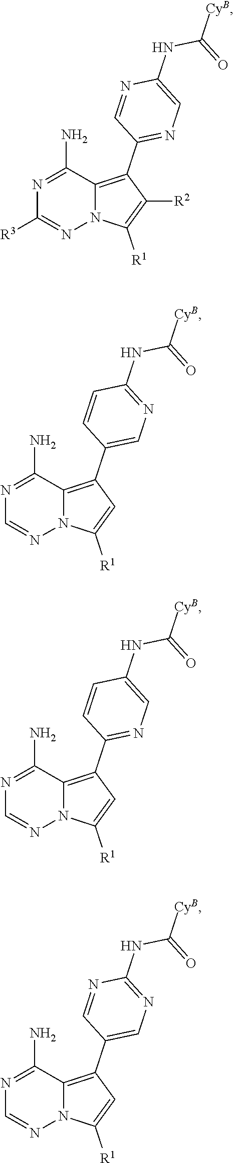

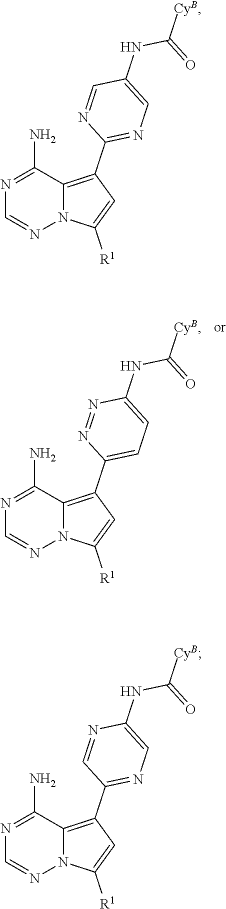

thereby forming a compound of Formulae I-ff-1, I-ff-2, I-ff-3, I-ff-4, I-ff-5, I-ff-6, I-ff-7, I-ff-8, I-ff-9, I-ff-10, I-ff-11, I-ff-12, I-ff-13, I-ff-14, I-ff-15, I-ff-16, I-ff-17, I-ff-18, I-ff-19, I-ff-20, I-ff-21, I-ff-22, I-ff-23, I-ff-24, I-ff-25, I-ff-26, I-ff-27, I-ff-28, or I-ff-29, respectively:

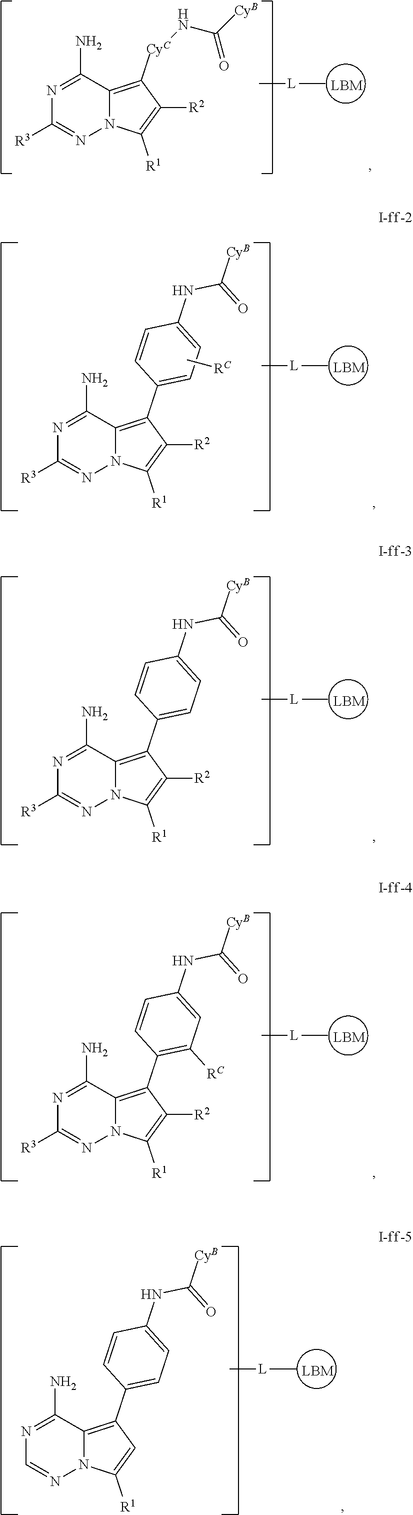

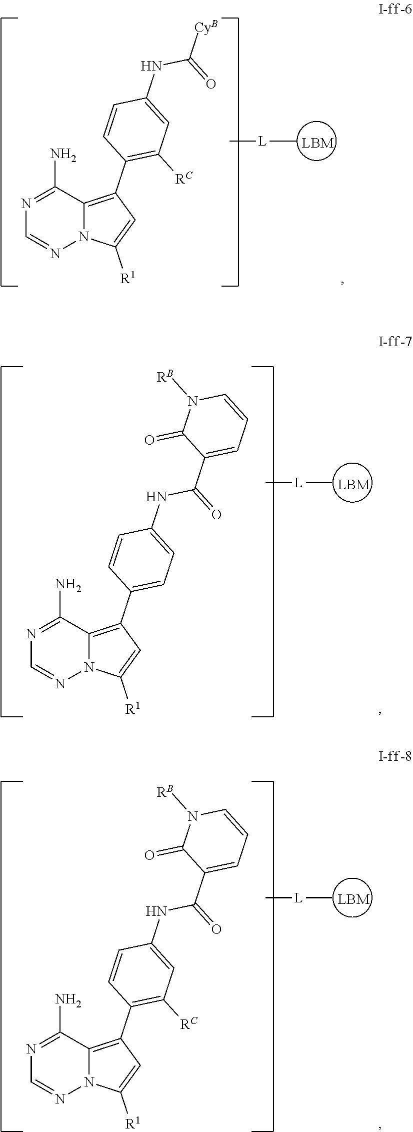

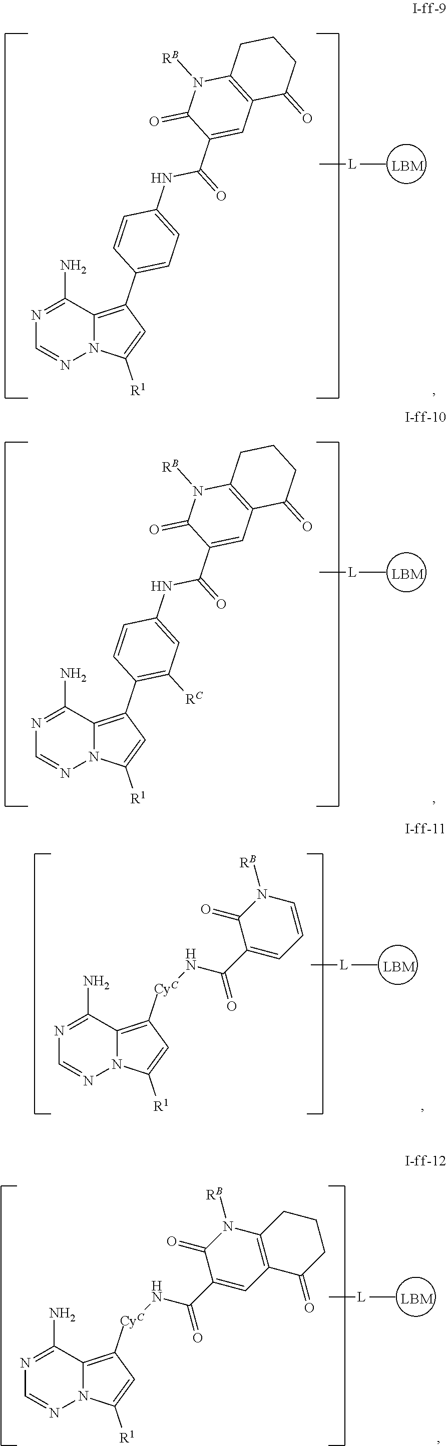

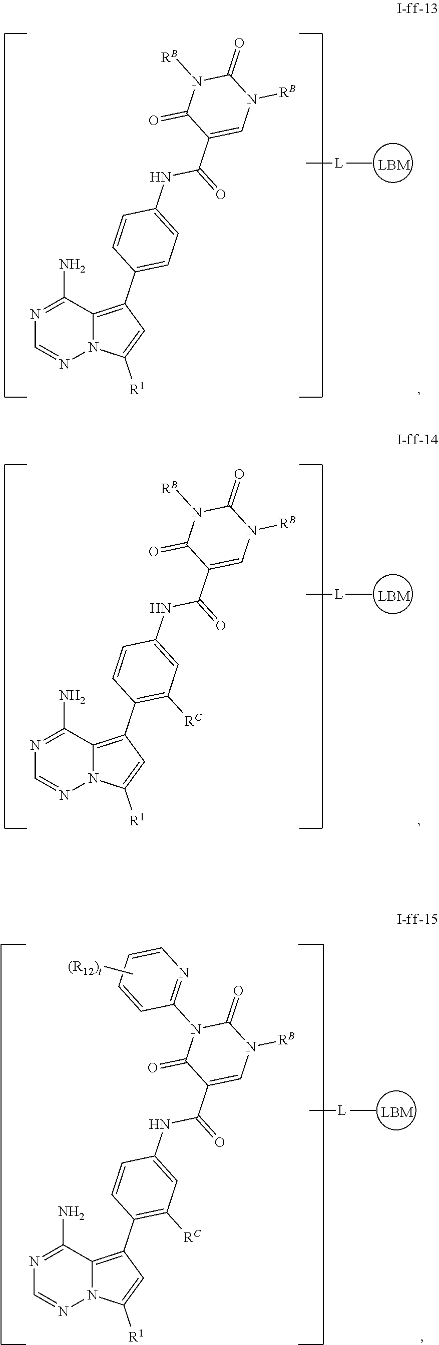

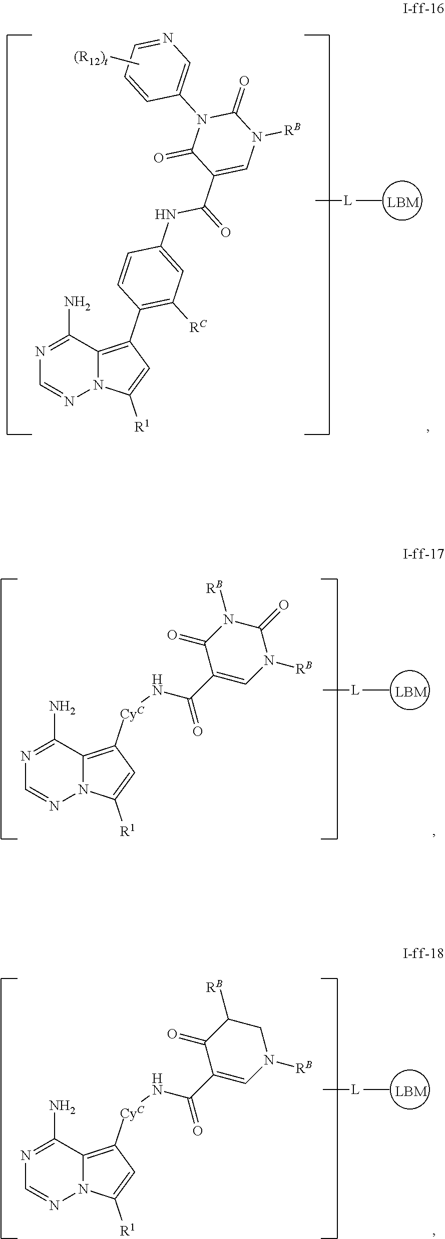

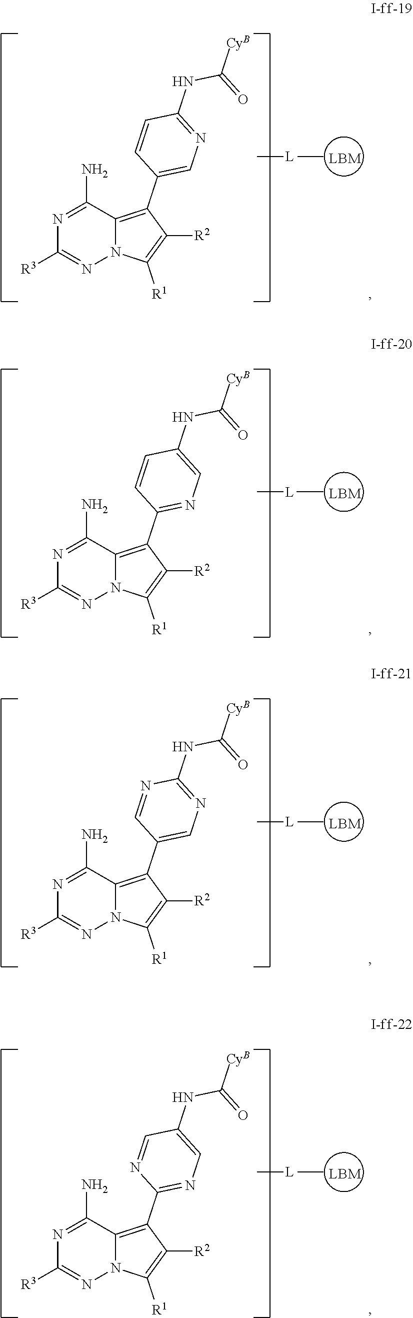

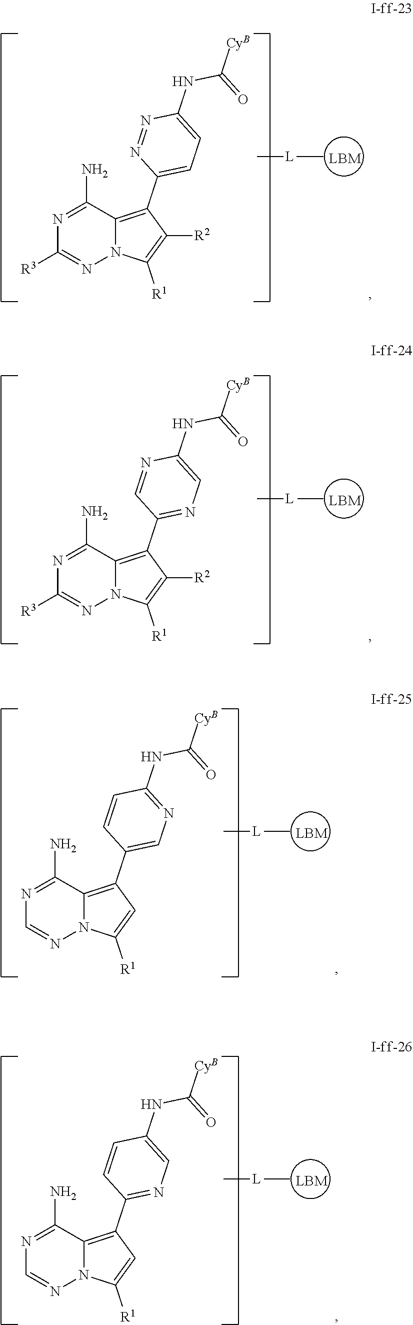

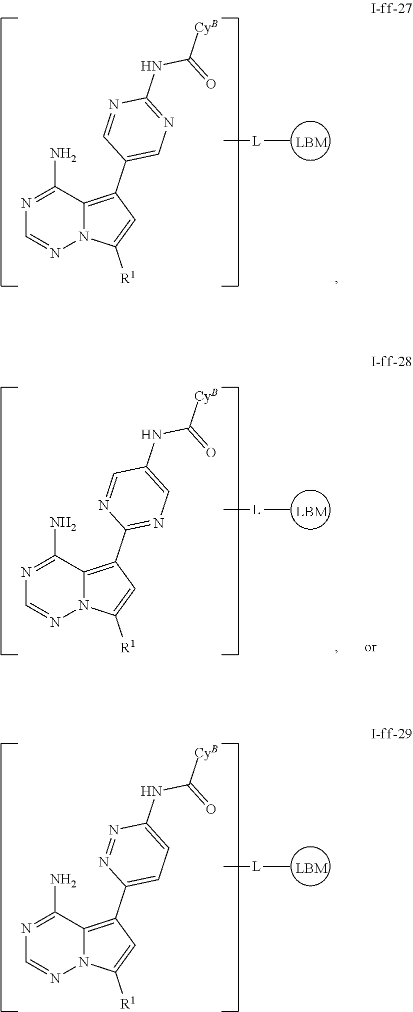

or a pharmaceutically acceptable salt thereof, wherein L and LBM are as defined above and described in embodiments herein, and wherein each of the variables Cy^{B}, Cy^{C}, R¹, R², R³, R^{B}, R^{C}, R₁₂, t, are as defined and described in U.S. Pat. App. Pub. No. 2017/0275290 and U.S. Pat. No. 9,981,975, which are incorporated herein by reference, in their entireties.

In certain embodiments, the present invention provides a compound of Formula I, wherein TAMBM is a TAM receptor kinase inhibitor,

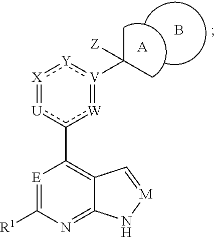

thereby forming a compound of Formula I-gg:

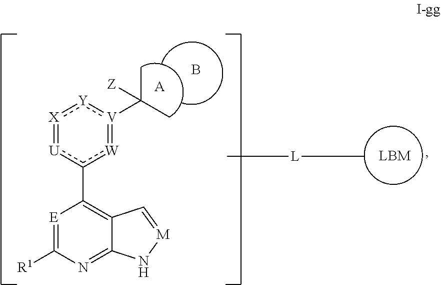

or a pharmaceutically acceptable salt thereof, wherein L and LBM are as defined above and described in embodiments herein, and wherein each of the variables Ring A, Ring B, R¹, E, N, U, V, W, X, Y, Z, M, are as defined and described in WO 2017/035366 and U.S. Pat. App. Pub. No. 2017/0057965, which are incorporated herein by reference, in their entireties.

In certain embodiments, the present invention provides a compound of Formula I, wherein TAMBM is an TAM kinase receptor inhibitor,

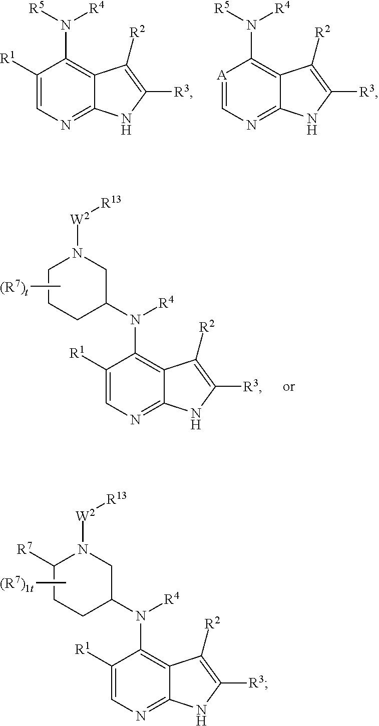

thereby forming a compound of Formulae I-hh-1, I-hh-2, I-hh-3, or I-hh-4, respectively:

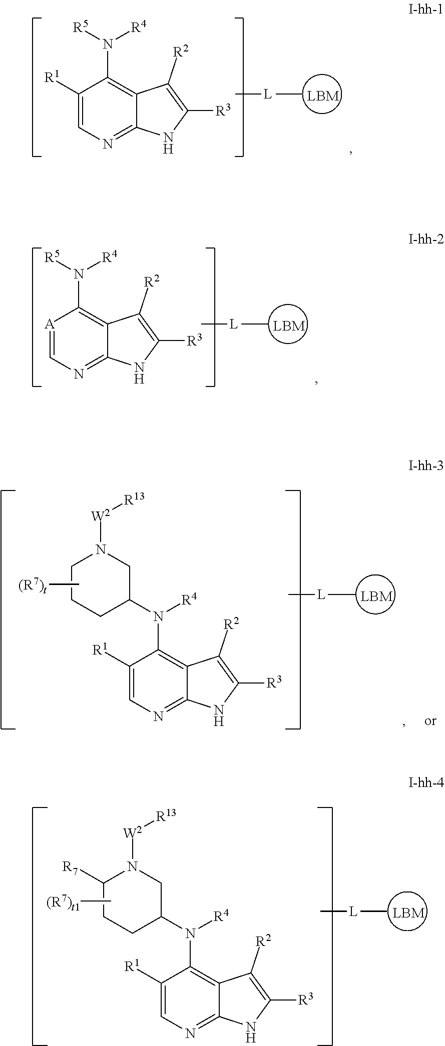

or a pharmaceutically acceptable salt thereof, wherein L and LBM are as defined above and described in embodiments herein, and wherein each of the variables R¹, R², R³, R⁴, R⁵, R⁷, R¹³, A, W², t, and t1, are as defined and described in U.S. Pat. No. 7,335,667, which is incorporated herein by reference, in its entirety.

In certain embodiments, the present invention provides a compound of Formula I, wherein TAMBM is a TAM receptor kinase inhibitor

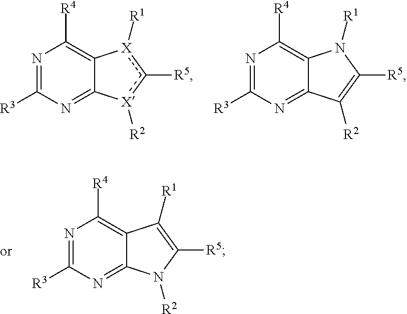

thereby forming a compound of Formulae I-ii-1, I-ii-2, or I-ii-3, respectively:

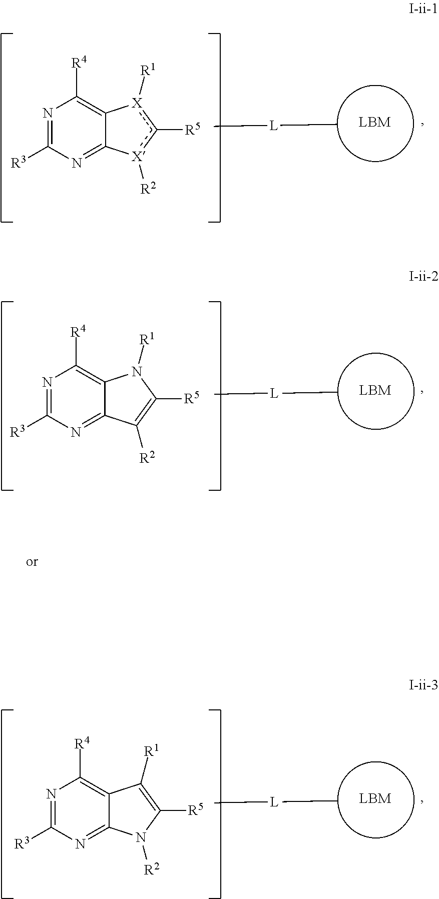

or a pharmaceutically acceptable salt thereof, wherein L and LBM are as defined above and described in embodiments herein, and wherein each of the variables, wherein each of R¹, R², R³, R⁴, R⁵, X and X′ are as defined and described in U.S. Pat. Nos. 9,555,031 and 9,273,056, which are incorporated herein, in their entireties.

In certain embodiments, the present invention provides a compound of Formula I, wherein TAMBM is a TAM receptor kinase inhibitor

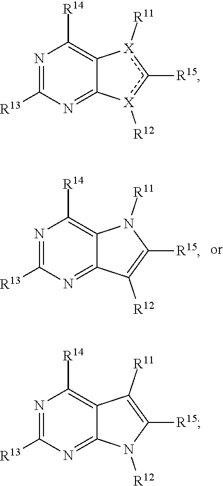

thereby forming a compound of Formulae I-ii-4, I-ii-5, or I-ii-6, respectively:

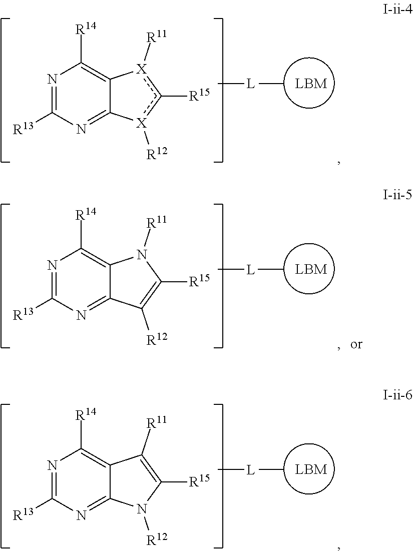

or a pharmaceutically acceptable salt thereof, wherein L and LBM are as defined above and described in embodiments herein, and wherein each of the variables Wherein, each of R¹¹, R¹², R¹³, R¹⁴, R¹⁵, X, and X′ are as defined and described in U.S. Pat. No. 9,555,031 and 9,273,056, which are incorporated herein, in their entireties.

In certain embodiments, the present invention provides a compound of Formula I, wherein TAMBM is a TAM receptor kinase inhibitor

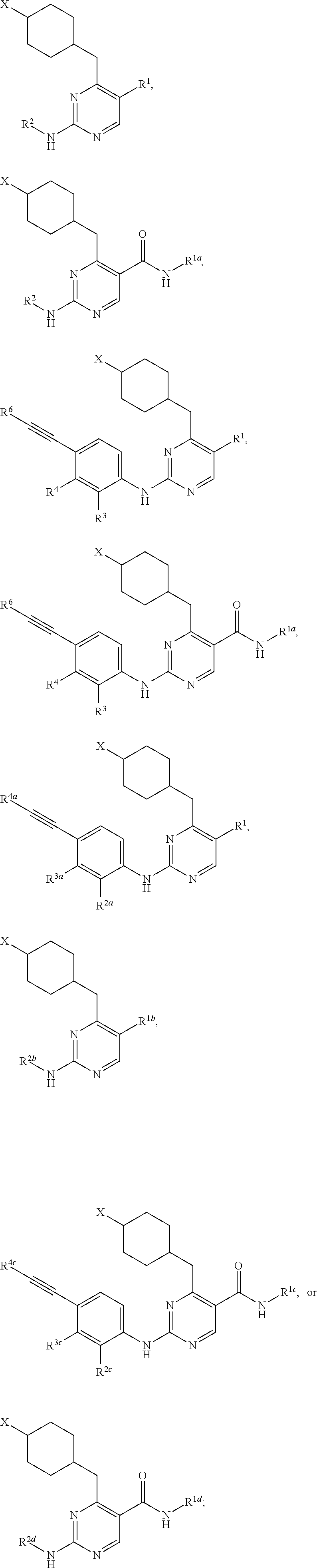

thereby forming a compound of Formulae I-jj-1, I-jj-2, I-jj-3, I-jj-4, I-jj-5, I-jj-6, I-jj-7, or I-jj-8, respectively:

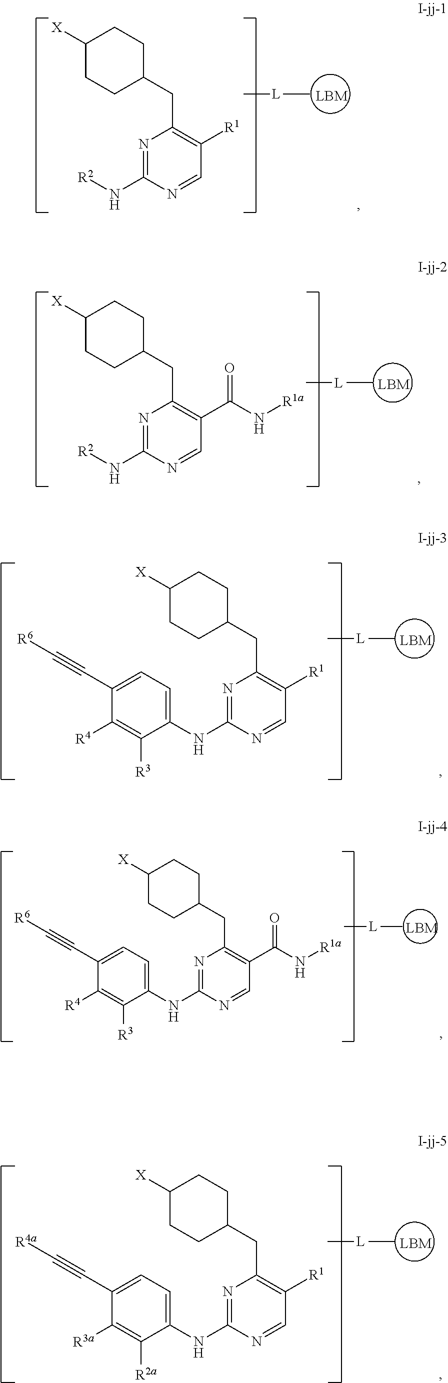

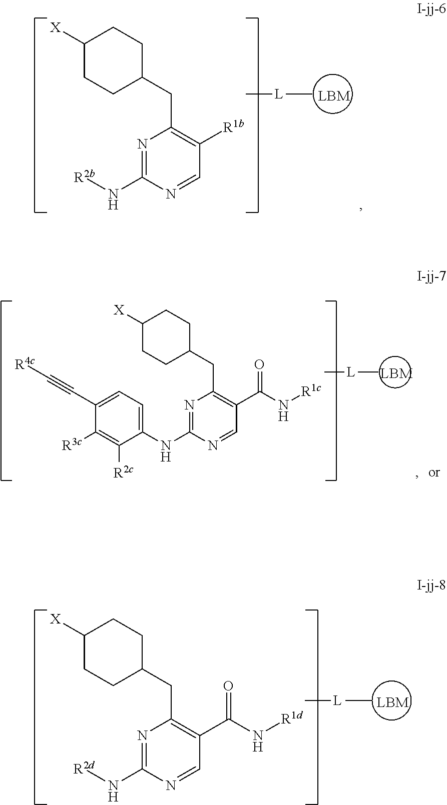

or a pharmaceutically acceptable salt thereof, wherein L and LBM are as defined above and described in embodiments herein, and wherein each of the variables X, R¹, R^{1a}, R^{1b}, R^{1c}, R^{1d}, R², R^{2a}, R^{2b}, R^{2c}, R^{2d}, R³, R^{3a}, R^{3c}, R⁴, R^{4a}, R^{4c}, and R⁶ are as defined and described in WO 2017/059280 and US 2018/0297977, which is incorporated herein, in its entirety.

In certain embodiments, the present invention provides a compound of Formula I, wherein TAMBM is a TAM receptor kinase inhibitor

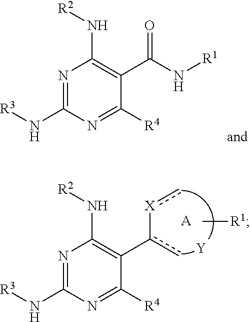

thereby forming a compound of Formulae I-kk-1 and I-kk-2, respectively:

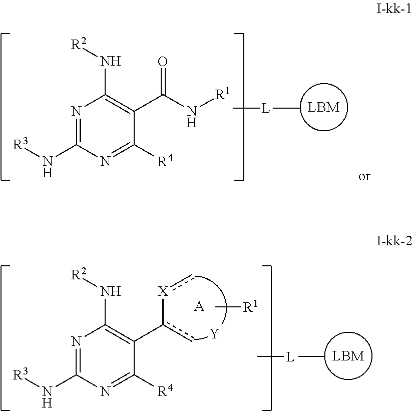

or a pharmaceutically acceptable salt thereof, wherein L and LBM are as defined above and described in embodiments herein, and wherein each of the variables R¹, R², R³, R⁴, Ring A, and Y are as defined and described in U.S. Pat. Nos. 9,567,326 and 9,771,330, which are incorporated herein by reference, in their entireties.

In certain embodiments, the present invention provides a compound of Formula I, wherein TAMBM is a TAM receptor kinase inhibitor

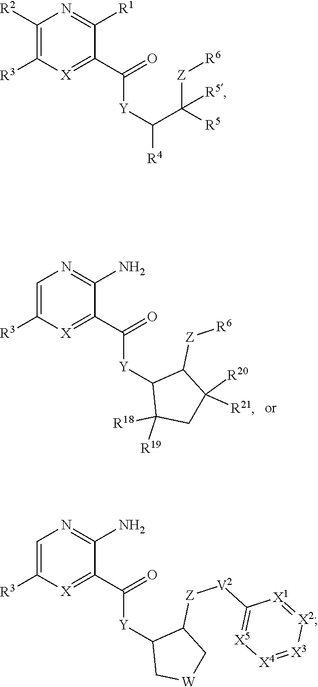

thereby forming a compound of Formulae I-ll-1, 1-ll-2, or 1-ll-3, respectively:

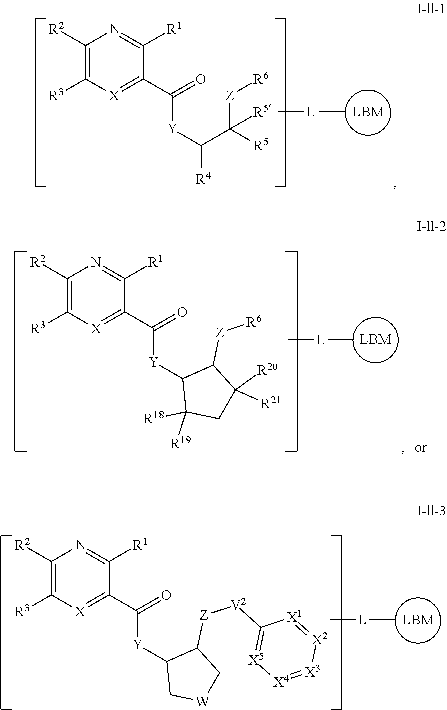

or a pharmaceutically acceptable salt thereof, wherein L and LBM are as defined above and described in embodiments herein, and wherein each of the variables R¹, R², R³, R⁴, R⁵, R^{5′}, R⁶, R¹⁸, R¹⁹, R²⁰, R²¹, R²¹, V¹, V², W, X, X¹, X², X³, X⁴, X⁵, Y, and Z, are as defined and described in U.S. Pat. App. Pub. No. 2017/0355690 and WO 2017/039331, which are incorporated herein by reference, in their entireties.

In certain embodiments, the present invention provides a compound of Formula I, wherein TAMBM is a TAM receptor kinase inhibitor,

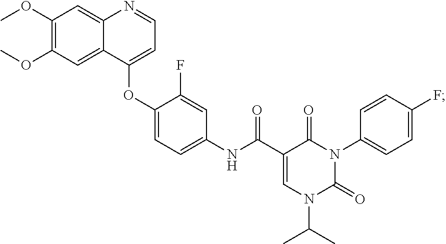 [SMILES: COc1cc2nccc(Oc3ccc(NC(=O)c4cn(C(C)C)c(=O)n(-c5ccc(F)cc5)c4=O)cc3F)c2cc1OC]

thereby forming a compound of Formula I-mm

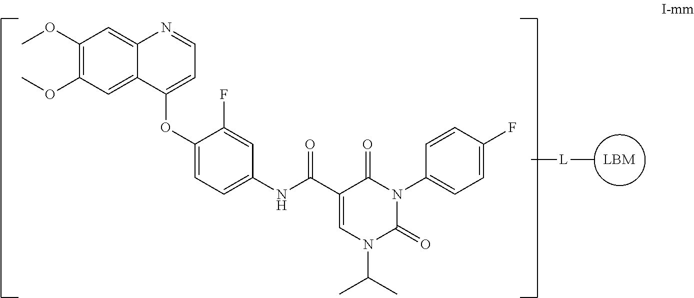

or a pharmaceutically acceptable salt thereof, wherein L and LBM are as defined above and described in embodiments herein, and wherein the TAMBM is as defined and described in WO 2015/017607, which is incorporated herein by reference, in its entirety.

In certain embodiments, the present invention provides a compound of Formula I, wherein TAMBM is a TAM receptor kinase inhibitor,

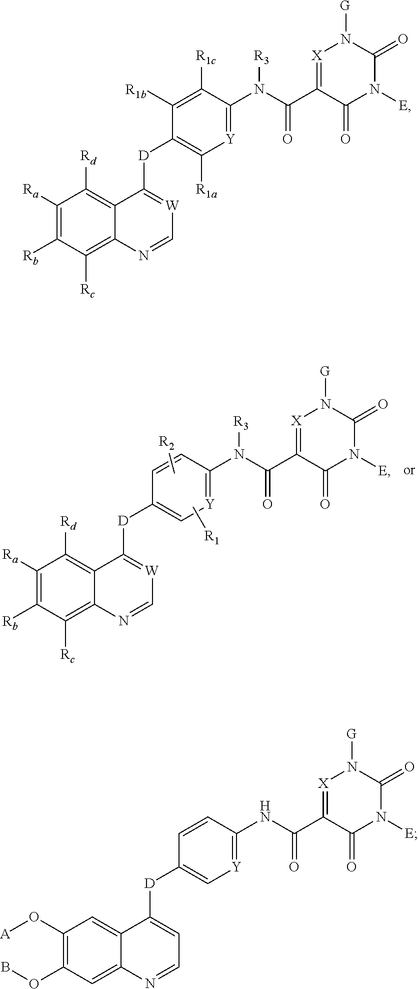

thereby forming a compound of Formulae I-nn-1, I-nn-2, or I-nn-3, respectively:

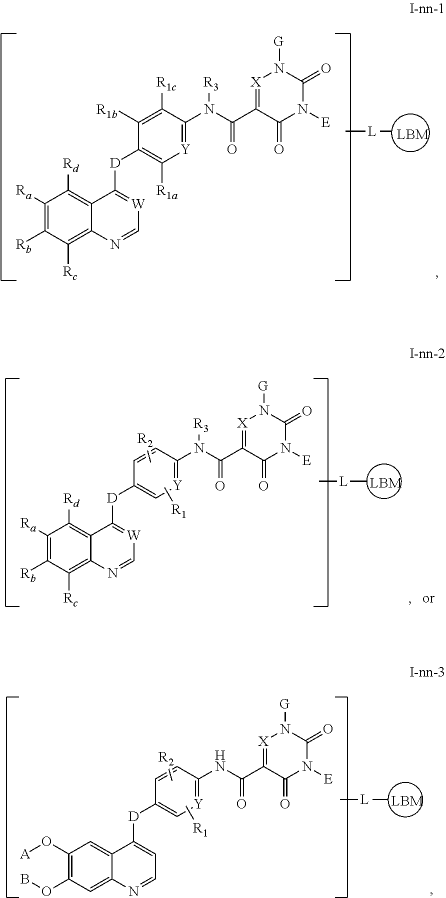

or a pharmaceutically acceptable salt thereof, wherein L and LBM are as defined above and described in embodiments herein, and wherein each of the variables R₁, R₂, R₃, R₁ₐ, R_{1b}, R_{1c}, Rₐ, R_{b}, R_{c}, R_{d}, D, E, G, X, and W, are as defined and described in U.S. Pat. No. 9,120,778, which is incorporated herein by reference, in its entirety.

In some embodiments, TAMBM is selected from a moiety recited in Yan, S. B. et al., LY2801653 is an orally bioavailable multi-kinase inhibitor with potent activity against MET, MST1R, and other oncoproteins, and displays anti-tumor activities in mouse xenograft models, Invest. New Drugs (2013) 31(4): 833-44, such as, for example:

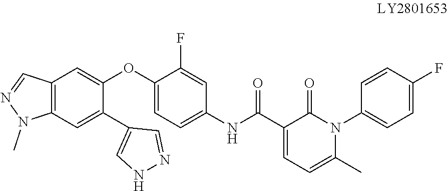 [SMILES: Cc1ccc(C(=O)Nc2ccc(Oc3cc4cnn(C)c4cc3-c3cn[nH]c3)c(F)c2)c(=O)n1-c1ccc(F)cc1]

wherein

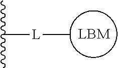

is attached to a modifiable carbon, oxygen, nitrogen or sulfur atom.

In some embodiments, TAMBM is selected from a moiety recited in Knubel, K. H. et al., MerTK inhibition is a novel therapeutic approach for glioblastoma multiforme, Oncotarget (2014) 5(5): 1338-51, such as, for example:

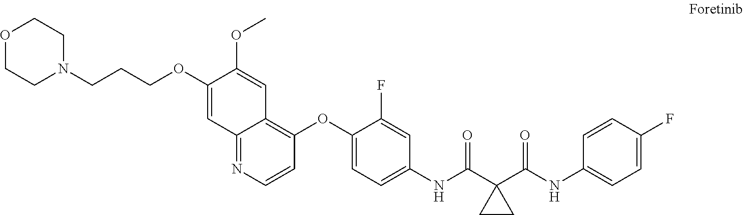 [SMILES: COc1cc2c(Oc3ccc(NC(=O)C4(C(=O)Nc5ccc(F)cc5)CC4)cc3F)ccnc2cc1OCCCN1CCOCC1]

wherein

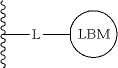

is attached to a modifiable carbon, oxygen, nitrogen or sulfur atom.

In some embodiments, TAMBM is selected from a moiety recited in Page, L. S. et al., MerTK Receptor Tyrosine Kinase Inhibition As a Potential Strategy to Augment Immune-Mediated Clearance of Acute Myeloid Leukemia, Blood (2016) 128(22): 4044-48; and/or Minson, K. A. et al., The MERTK/FLT3 inhibitor MRX-2843 overcomes resistance-conferring FLT3 mutations in acute myeloid leukemia, JCI Insight. (2016) 1(3): e85630, https://doi.org/10.1172/jci.insight.85630, such as, for example:

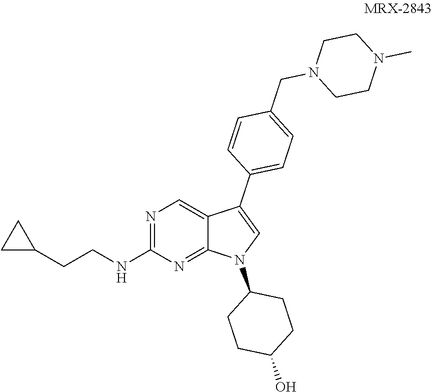 [SMILES: CN1CCN(Cc2ccc(-c3cn([C@H]4CC[C@H](O)CC4)c4nc(NCCC5CC5)ncc34)cc2)CC1]

wherein

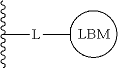

is attached to a modifiable carbon, oxygen, nitrogen or sulfur atom.

In some embodiments, TAMBM is selected from a moiety recited in Kim, J. E. et al., MerTK inhibition by RXDX-106 in MerTK activated gastric cancer cell lines, Oncotarget (2017) 8(62): 105727-34, such as, for example:

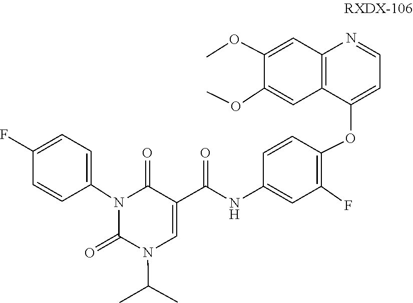 [SMILES: COc1cc2nccc(Oc3ccc(NC(=O)c4cn(C(C)C)c(=O)n(-c5ccc(F)cc5)c4=O)cc3F)c2cc1OC]

wherein

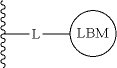

is attached to a modifiable carbon, oxygen, nitrogen or sulfur atom.

In some embodiments, TAMBM is selected from a moiety recited in Zhang, W. et al., UNC2025, a Potent and Orally Bioavailable MER/FLT3 Dual Inhibitor, J. Med. Chem. (2014) 57(16): 7031-41, such as, for example:

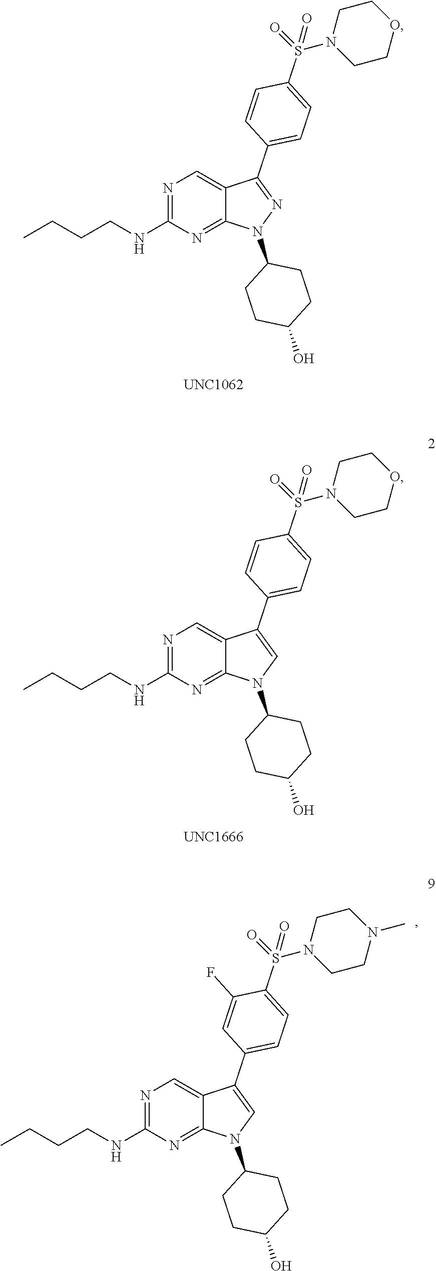 [SMILES: CCCCNc1ncc2c(-c3ccc(S(=O)(=O)N4CCOCC4)cc3)nn([C@H]3CC[C@H](O)CC3)c2n1 ; CCCCNc1ncc2c(-c3ccc(S(=O)(=O)N4CCOCC4)cc3)cn([C@H]3CC[C@H](O)CC3)c2n1 ; CCCCNc1ncc2c(-c3ccc(S(=O)(=O)N4CCN(C)CC4)c(F)c3)cn([C@H]3CC[C@H](O)CC3)c2n1]

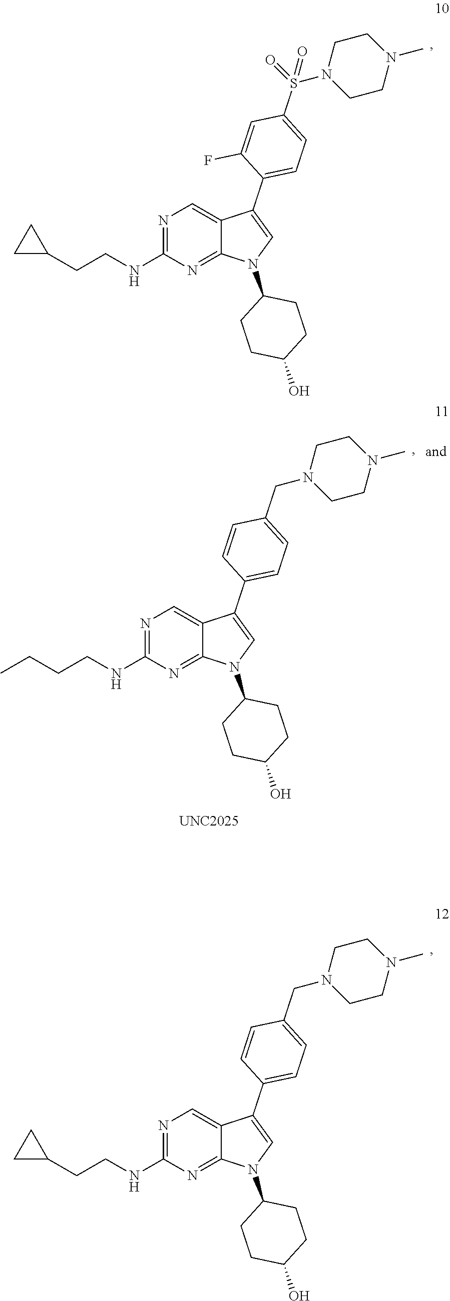

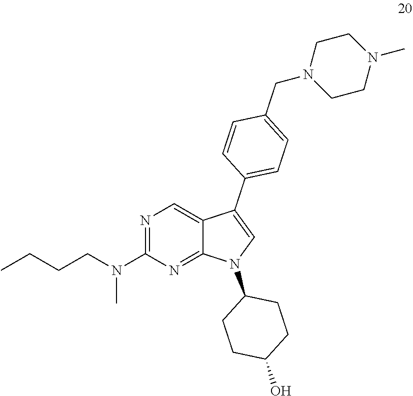 [SMILES: CCCCN(C)c1ncc2c(-c3ccc(CN4CCN(C)CC4)cc3)cn([C@H]3CC[C@H](O)CC3)c2n1]

wherein

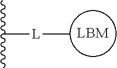

is attached to a modifiable carbon, oxygen, nitrogen or sulfur atom.

In some embodiments, TAMBM is selected from a moiety recited in Huey, M. G. et al., Targeting the TAM Receptors in Leukemia, Cancers (2016) 8(11): 101/1-101/22, such as, for example:

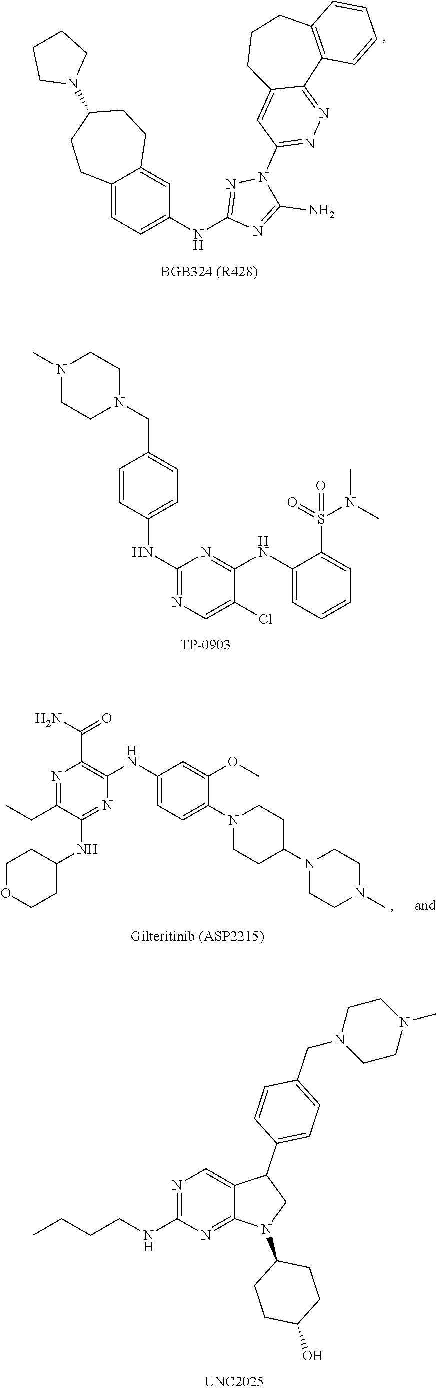

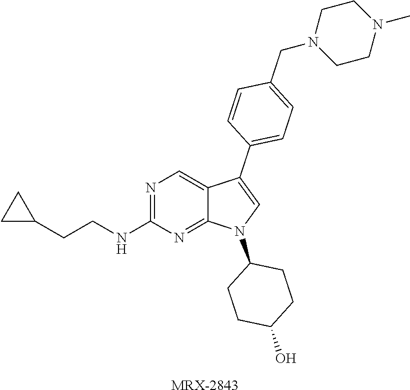

wherein

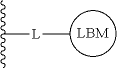

is attached to a modifiable carbon, oxygen, nitrogen or sulfur atom.

In some embodiments, TAMBM is selected from a moiety recited in DeRyckere, A. B. et al., UNC2025, a MERTK Small-Molecule Inhibitor, Is Therapeutically Effective Alone and in Combination with Methotrexate in Leukemia Models, Clin. Cancer Res. (2017) 23(6): 1481-92; Sufit, A. et al., MERTK Inhibition Induces Polyploidy and Promotes Cell Death and Cellular Senescence in Glioblastoma Multiforme, PLoS One (2016) 11(10); e0165107/1-20; Cummings, C. T. et al., Small Molecule Inhibition of MERTK is Efficacious in Non-Small Cell lung Cancer Models Independent of Driver Oncogene Status, Mol. Cancer. Ther. (2015) 14(9): 2014-22; Shi, C. et al., The proto-oncogene Mer tyrosine kinase is a novel therapeutic target in mantle cell lymphoma, J. Hematol. Oncol. (2018) 11(1): 43; and Branchford, B. R. et al., The small-molecule MERTK inhibitor UNC2025 decreases platelet activation and prevents thrombosis, J. Thromb. Haemost. (2018) 16(2): 352-63 such as, for example:

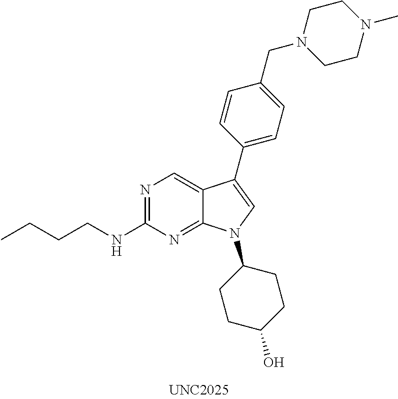 [SMILES: CCCCNc1ncc2c(-c3ccc(CN4CCN(C)CC4)cc3)cn([C@H]3CC[C@H](O)CC3)c2n1]

wherein

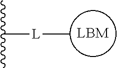

is attached to a modifiable carbon, oxygen, nitrogen or sulfur atom.

In some embodiments, TAIVIBM is selected from a moiety recited in Paolino, M. et al., The E3 ligase Cbl-b and TAM receptors regulate cancer metastasis via natural killer cells, Nature (2014) 507(7493): 508-12, such as, for example:

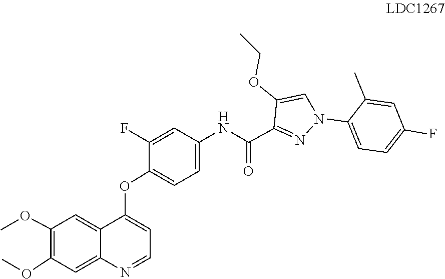 [SMILES: CCOc1cn(-c2ccc(F)cc2C)nc1C(=O)Nc1ccc(Oc2ccnc3cc(OC)c(OC)cc23)c(F)c1]

wherein

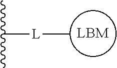

is attached to a modifiable carbon, oxygen, nitrogen or sulfur atom.

In some embodiments, TAMBM is selected from a moiety recited in Cummings, C. T. et al., Molecular Pathways: MERTK Signaling in Cancer, Clin. Cancer Res. (2013) 19(19): 5275-80, such as, for example:

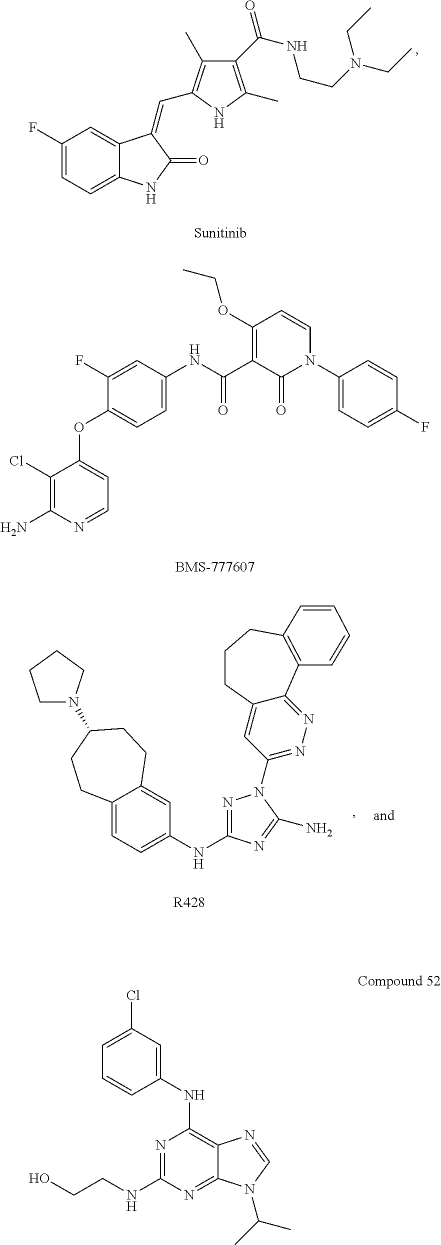

wherein

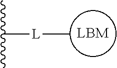

is attached to a modifiable carbon, oxygen, nitrogen or sulfur atom.

In some embodiments, TAMBM is selected from a moiety recited in Schlegel, J. et al., MERTK receptor tyrosine kinase is a therapeutic target in melanoma, J. Clin. Invest. (2013) 123(5): 2257-67; and Ho, Y. J. et al., MerTK is a novel therapeutic target in gastric cancer, Oncotarget (2017) 8(57): 96656-67, such as, for example:

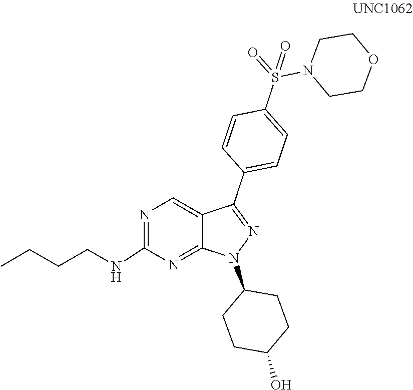 [SMILES: CCCCNc1ncc2c(-c3ccc(S(=O)(=O)N4CCOCC4)cc3)nn([C@H]3CC[C@H](O)CC3)c2n1]

wherein

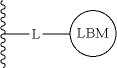

is attached to a modifiable carbon, oxygen, nitrogen or sulfur atom.

In some embodiments, TAMBM is selected from a moiety recited in Espindola, M. S. et al., Targeting TAM Receptors Ameliorates Fibrotic Mechanisms in Idiopathic Pulmonary Fibrosis, Am. J. Resp. Critical Med. (2018) 197(11): 1443-56; and Mikaella, V. & Sassan, H., TAM Receptor Tyrosine Kinases in Cancer Drug Resistance, Cancer Res. (2017) 77(11): 2775-8, such as, for example:

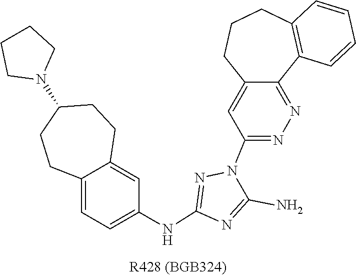 [SMILES: Nc1nc(Nc2ccc3c(c2)CC[C@@H](N2CCCC2)CC3)nn1-c1cc2c(nn1)-c1ccccc1CCC2]

wherein

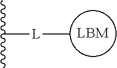

is attached to a modifiable carbon, oxygen, nitrogen or sulfur atom.

In some embodiments, TAMBM is selected from a moiety recited in Kimani, S. G. et al., Small molecule inhibitors block Gas6-inducible TAM activation and tumorigenicity, Scientific Reports (2017) 7: 74908, such as, for example:

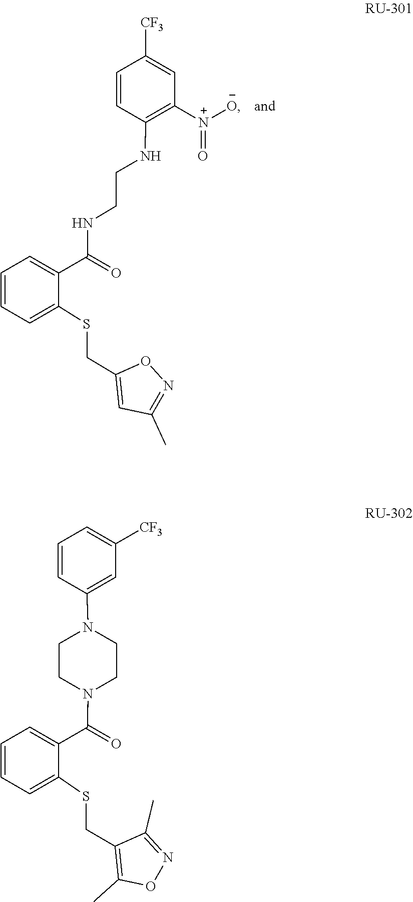

wherein

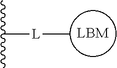

is attached to a modifiable carbon, oxygen, nitrogen or sulfur atom.

In some embodiments, TAMBM is selected from a moiety recited in Schoumacher, M. & Burbridge, M., Key Roles of AXL and MER Receptor Tyrosine Kinases in Resistance to Multiple Anticancer Therapies, Curr. Oncol. Rep. (2017) 19(3):19, such as, for example:

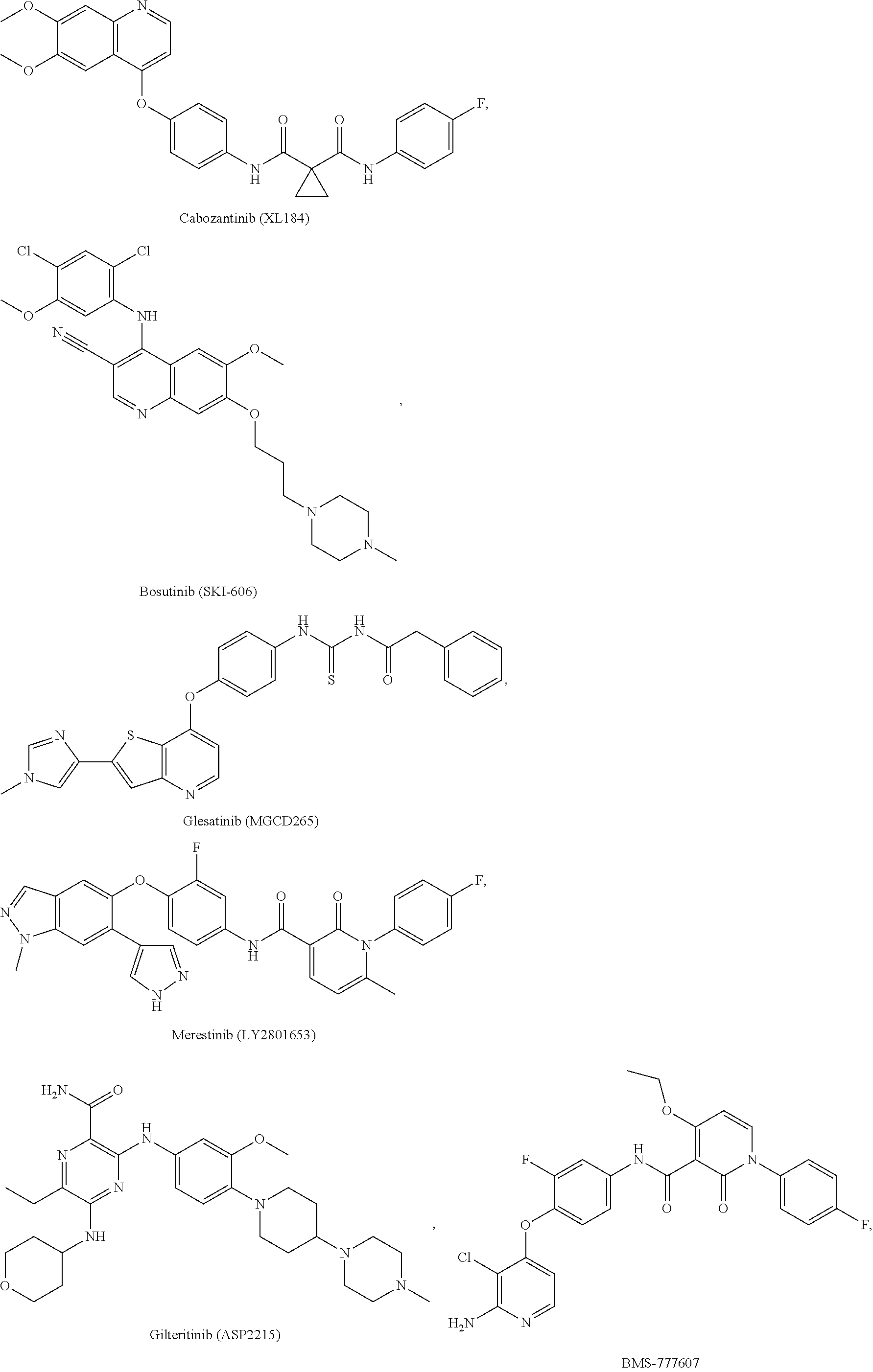

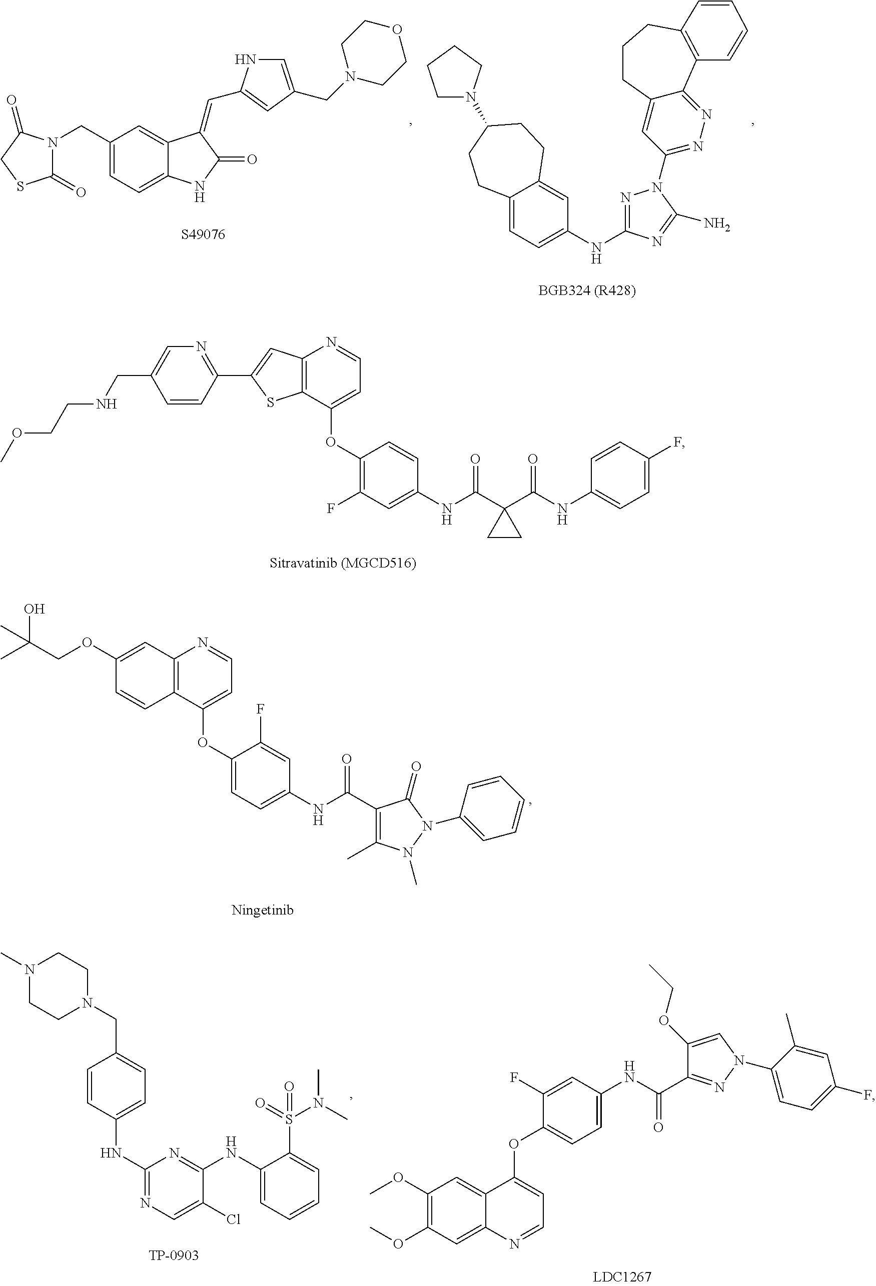

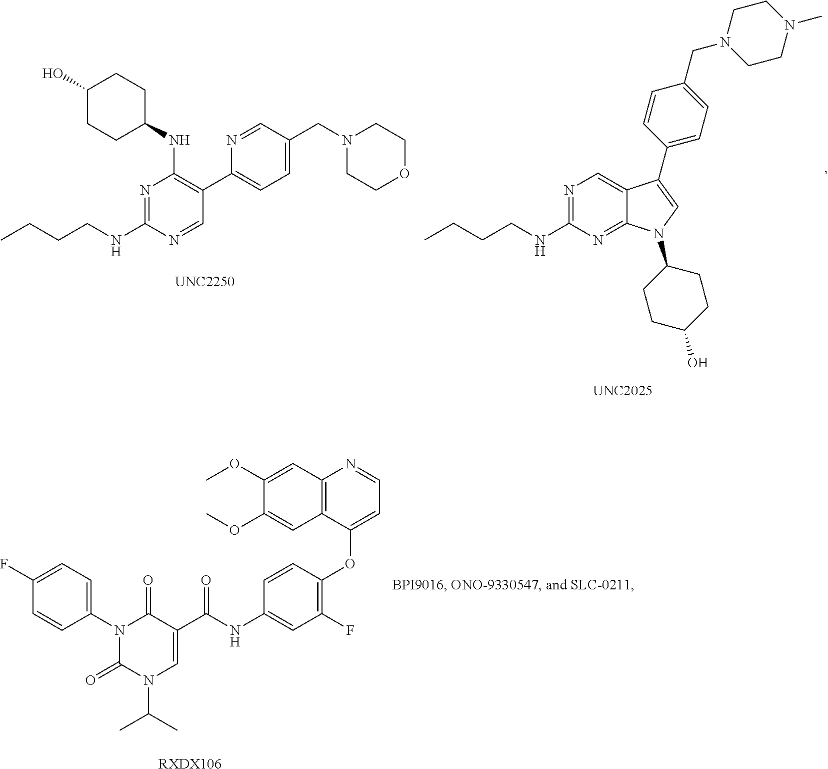

wherein

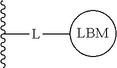

is attached to a modifiable carbon, oxygen, nitrogen or sulfur atom.

In some embodiments, TAMBM is selected from a moiety recited in Davra, V. et al., Ligand Activation of TAM Family Receptors-Implications for Tumor Biology and Therapeutic Response, Cancers (Basel) (2016) 8(12): 107, such as, for example:

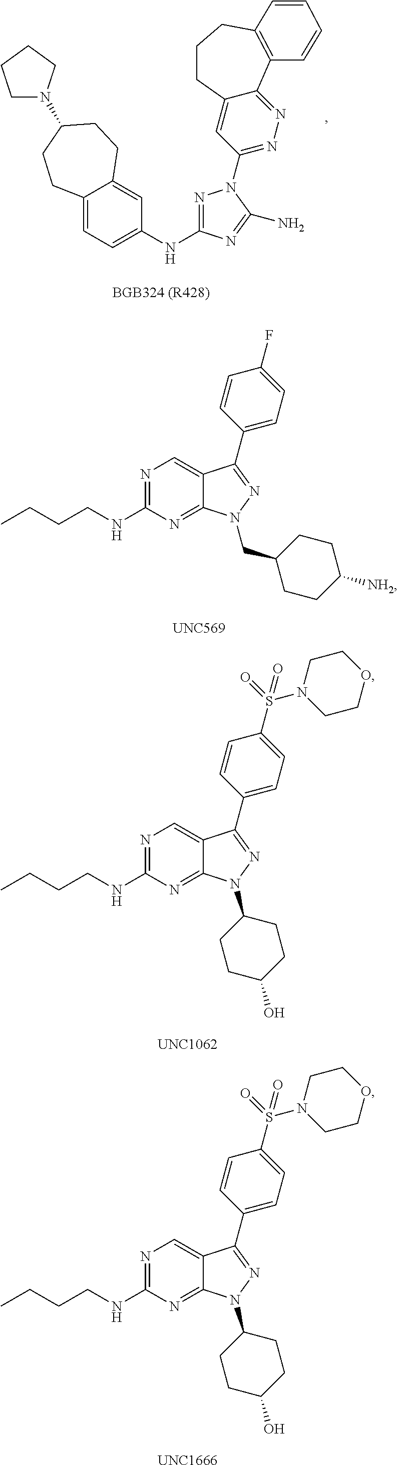

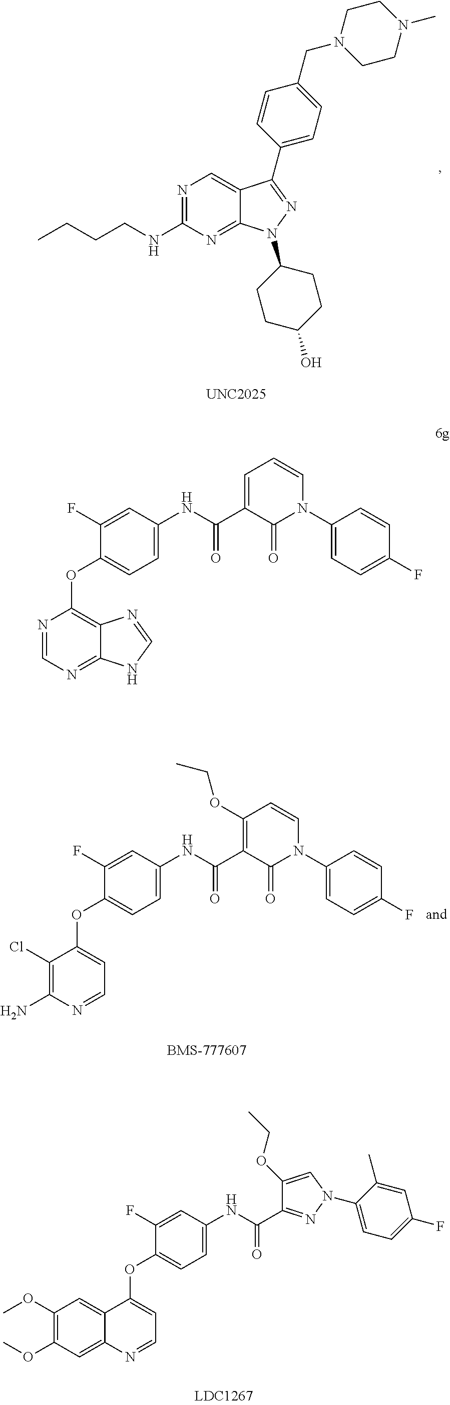

wherein

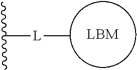

is attached to a modifiable carbon, oxygen, nitrogen or sulfur atom.

In some embodiments, TAMBM is selected from a moiety recited in von Massenhaausen, A. et al., MERTK as a novel therapeutic target in head and neck cancer, Oncotarget (2016) 7(22): 32678-94, such as, for example:

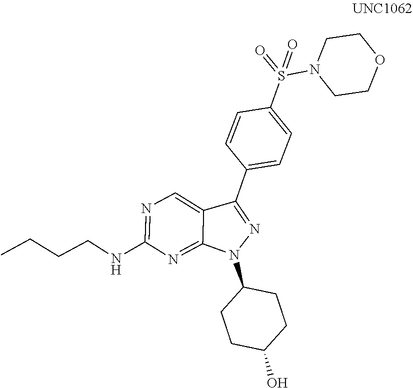 [SMILES: CCCCNc1ncc2c(-c3ccc(S(=O)(=O)N4CCOCC4)cc3)nn([C@H]3CC[C@H](O)CC3)c2n1]

wherein

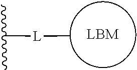

is attached to a modifiable carbon, oxygen, nitrogen or sulfur atom.

In some embodiments, TAMBM is selected from a moiety recited in Lee-Sherick, A. B. et al., Efficacy of a Mer and Flt3 tyrosine kinase small molecule inhibitor, UNC1666, in acute myeloid leukemia, Oncotarget (2015) 6(9): 6722-36, such as, for example:

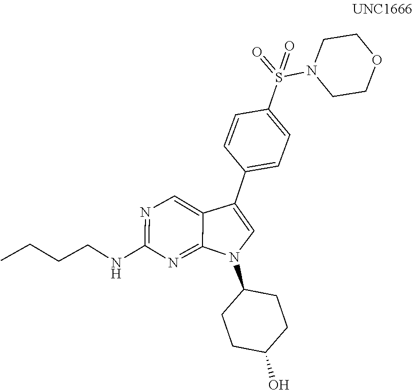 [SMILES: CCCCNc1ncc2c(-c3ccc(S(=O)(=O)N4CCOCC4)cc3)cn([C@H]3CC[C@H](O)CC3)c2n1]

wherein

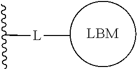

is attached to a modifiable carbon, oxygen, nitrogen or sulfur atom.

In some embodiments, TAMBM is selected from a moiety recited in Christoph, S. et al., UNC569, a novel small-molecule mer inhibitor with efficacy against acute lymphoblastic leukemia in vitro and in vivo, Mol. Cancer. Ther. (2013) 12(11): 2367-77, such as, for example:

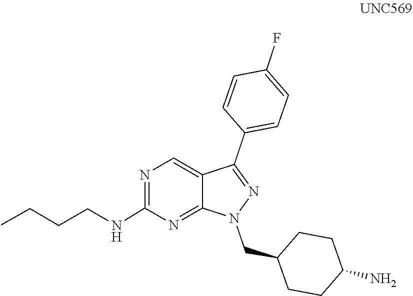 [SMILES: CCCCNc1ncc2c(-c3ccc(F)cc3)nn(C[C@H]3CC[C@H](N)CC3)c2n1]

wherein

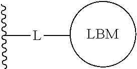

is attached to a modifiable carbon, oxygen, nitrogen or sulfur atom.

In some embodiments, TAMBM is selected from a moiety recited in Liu, J. et al., UNC1062, a new and potent Mer inhibitor, Eur. J. Med. Chem. (2013) 65: 83-93, such as, for example:

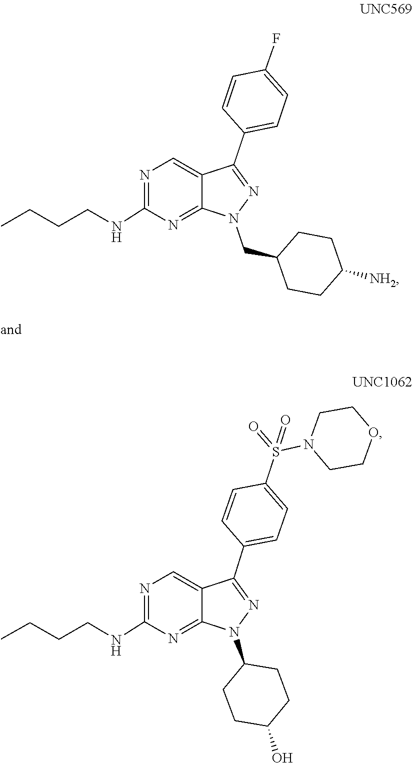 [SMILES: CCCCNc1ncc2c(-c3ccc(F)cc3)nn(C[C@H]3CC[C@H](N)CC3)c2n1 ; CCCCNc1ncc2c(-c3ccc(S(=O)(=O)N4CCOCC4)cc3)nn([C@H]3CC[C@H](O)CC3)c2n1]

wherein

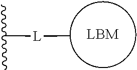

is attached to a modifiable carbon, oxygen, nitrogen or sulfur atom.

In some embodiments, TAMBM is selected from a moiety recited in Suarez, R. M. et al., Inhibitors of the TAM subfamily of tyrosine kinases: Synthesis and biological evaluation, Eur. J. Med. Chem (2013) 61: 2-25, such as, for example:

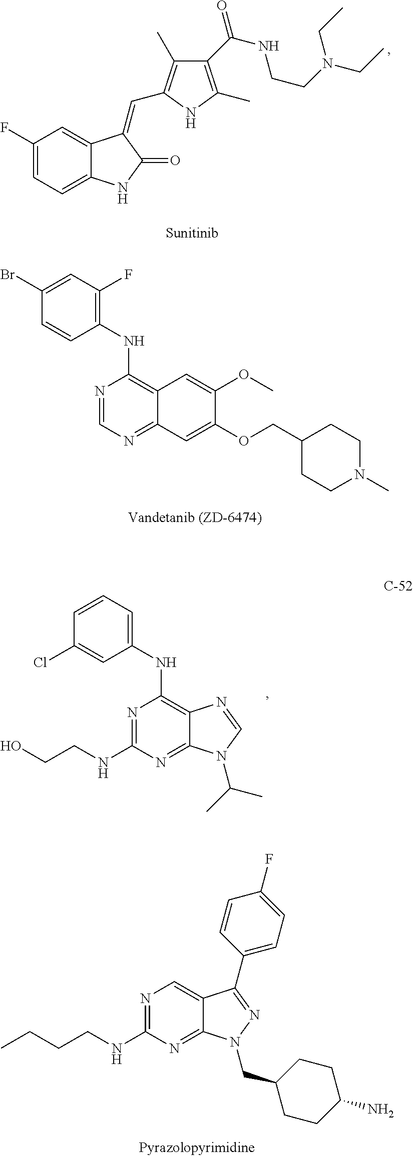

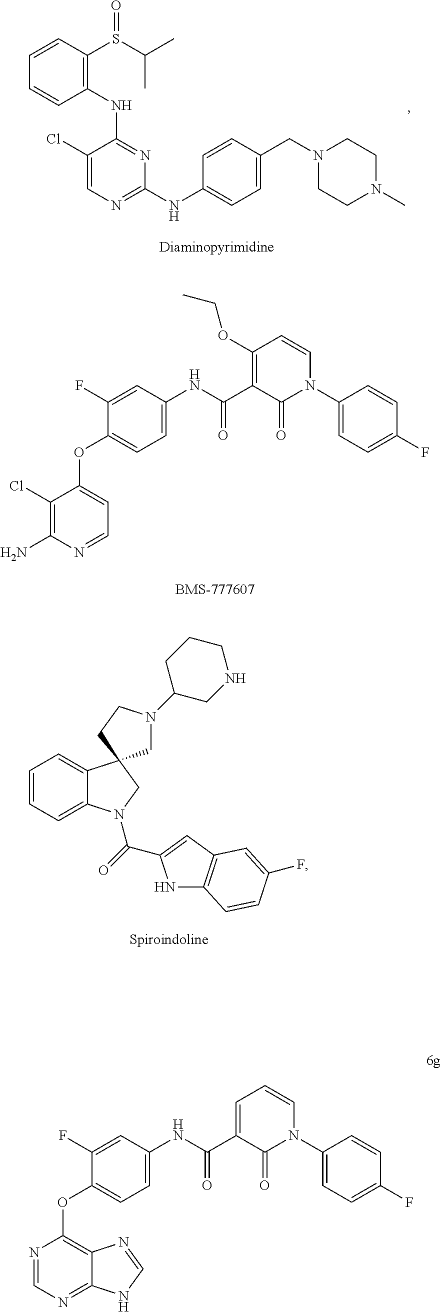 [SMILES: CC(C)S(=O)c1ccccc1Nc1nc(Nc2ccc(CN3CCN(C)CC3)cc2)ncc1Cl ; CCOc1ccn(-c2ccc(F)cc2)c(=O)c1C(=O)Nc1ccc(Oc2ccnc(N)c2Cl)c(F)c1 ; O=C(c1cc2cc(F)ccc2[nH]1)N1C[C@]2(CCN(C3CCCNC3)C2)c2ccccc21 ; O=C(Nc1ccc(Oc2ncnc3[nH]cnc23)c(F)c1)c1cccn(-c2ccc(F)cc2)c1=O]

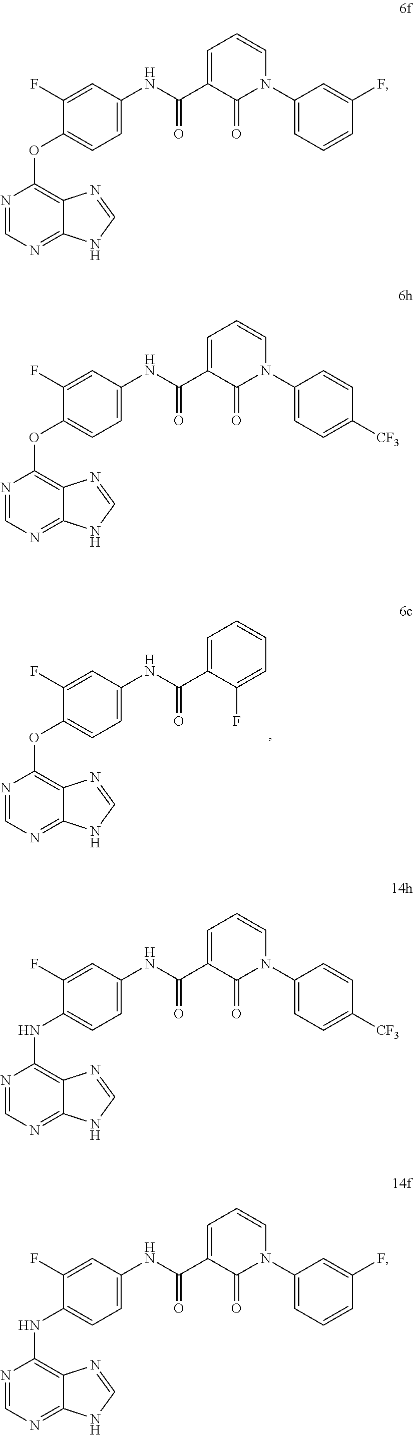 [SMILES: O=C(Nc1ccc(Oc2ncnc3[nH]cnc23)c(F)c1)c1cccn(-c2cccc(F)c2)c1=O ; O=C(Nc1ccc(Oc2ncnc3[nH]cnc23)c(F)c1)c1cccn(-c2ccc(C(F)(F)F)cc2)c1=O ; O=C(Nc1ccc(Oc2ncnc3[nH]cnc23)c(F)c1)c1ccccc1F ; O=C(Nc1ccc(Nc2ncnc3[nH]cnc23)c(F)c1)c1cccn(-c2ccc(C(F)(F)F)cc2)c1=O ; O=C(Nc1ccc(Nc2ncnc3[nH]cnc23)c(F)c1)c1cccn(-c2cccc(F)c2)c1=O]

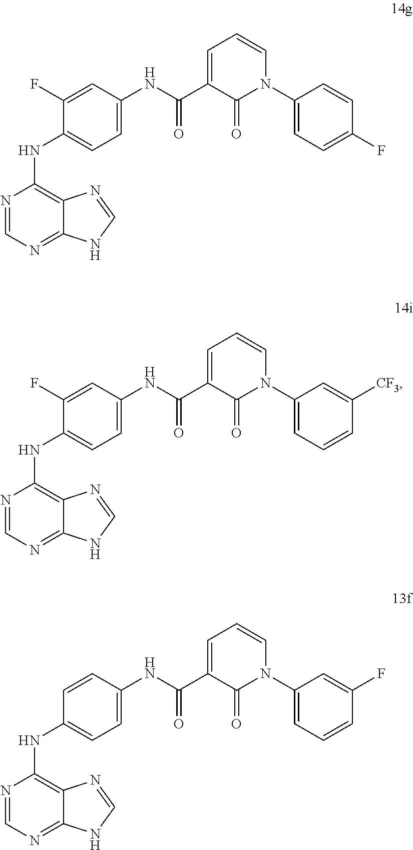 [SMILES: O=C(Nc1ccc(Nc2ncnc3[nH]cnc23)c(F)c1)c1cccn(-c2ccc(F)cc2)c1=O ; O=C(Nc1ccc(Nc2ncnc3[nH]cnc23)c(F)c1)c1cccn(-c2cccc(C(F)(F)F)c2)c1=O ; O=C(Nc1ccc(Nc2ncnc3[nH]cnc23)cc1)c1cccn(-c2cccc(F)c2)c1=O]

wherein

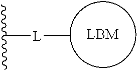

is attached to a modifiable carbon, oxygen, nitrogen or sulfur atom.

In some embodiments, TAMBM is

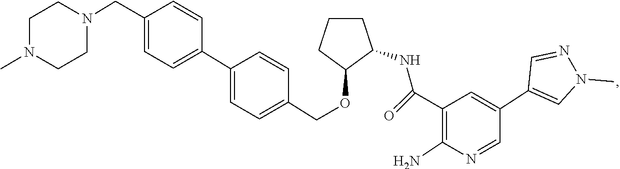

wherein

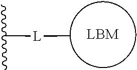

is attached to a modifiable carbon, oxygen, nitrogen or sulfur atom.

In some embodiments, TAMBM is

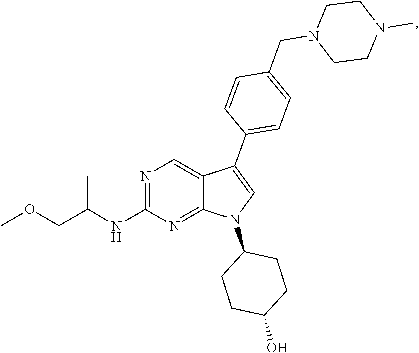

wherein

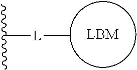

is attached to a modifiable carbon, oxygen, nitrogen or sulfur atom.

In some embodiments, TAMBM is

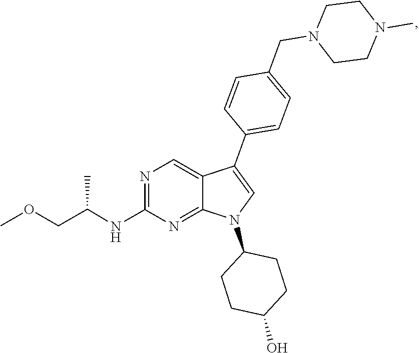

wherein

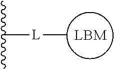

is attached to a modifiable carbon, oxygen, nitrogen or sulfur atom.

In some embodiments, TAMBM is

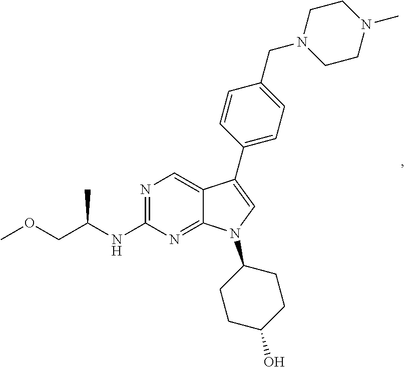

wherein

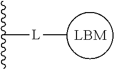

is attached to a modifiable carbon, oxygen, nitrogen or sulfur atom.

In some embodiments, TAMBM is

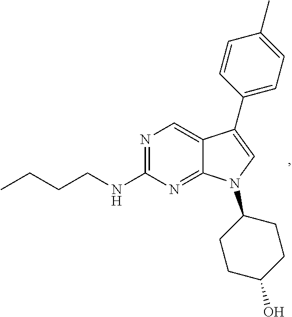 [SMILES: CCCCNc1ncc2c(-c3ccc(C)cc3)cn([C@H]3CC[C@H](O)CC3)c2n1]

wherein

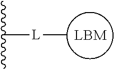

is attached to a modifiable carbon, oxygen, nitrogen or sulfur atom.

In some embodiments, TAMBM is

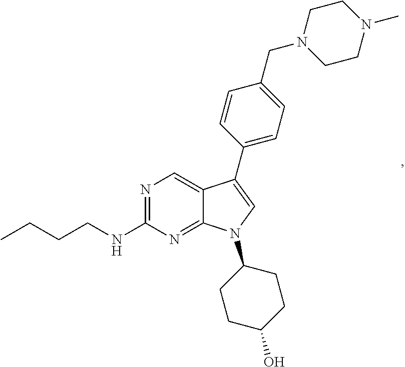 [SMILES: CCCCNc1ncc2c(-c3ccc(CN4CCN(C)CC4)cc3)cn([C@H]3CC[C@H](O)CC3)c2n1]

wherein

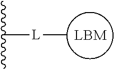

is attached to a modifiable carbon, oxygen, nitrogen or sulfur atom.

In some embodiments, TAMBM is

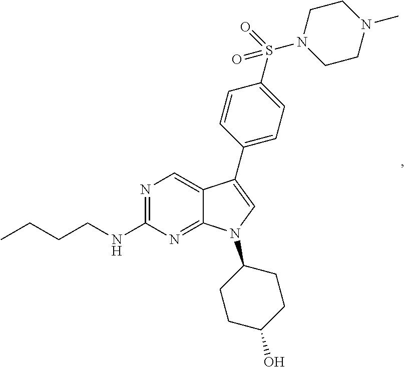 [SMILES: CCCCNc1ncc2c(-c3ccc(S(=O)(=O)N4CCN(C)CC4)cc3)cn([C@H]3CC[C@H](O)CC3)c2n1]

wherein

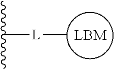

is attached to a modifiable carbon, oxygen, nitrogen or sulfur atom.

In some embodiments, TAMBM is

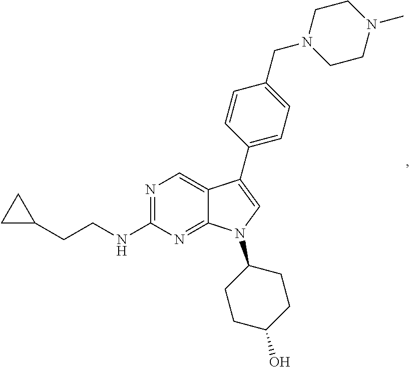 [SMILES: CN1CCN(Cc2ccc(-c3cn([C@H]4CC[C@H](O)CC4)c4nc(NCCC5CC5)ncc34)cc2)CC1]

wherein

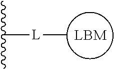

is attached to a modifiable carbon, oxygen, nitrogen or sulfur atom.

In some embodiments, TAMBM is

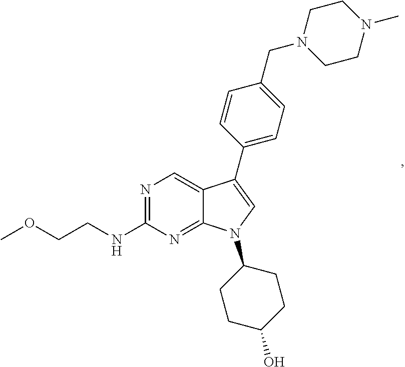

wherein

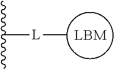

is attached to a modifiable carbon, oxygen, nitrogen or sulfur atom.

In some embodiments, TAMBM is

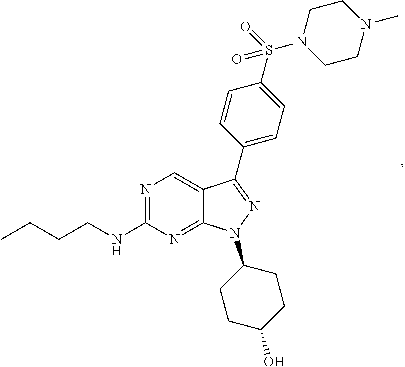 [SMILES: CCCCNc1ncc2c(-c3ccc(S(=O)(=O)N4CCN(C)CC4)cc3)nn([C@H]3CC[C@H](O)CC3)c2n1]

wherein

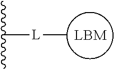

is attached to a modifiable carbon, oxygen, nitrogen or sulfur atom.

In some embodiments, TAMBM is

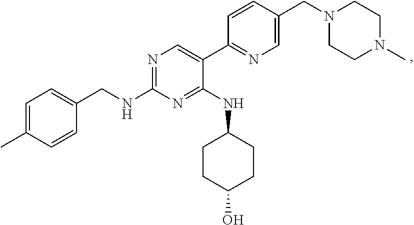

wherein

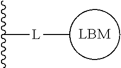

is attached to a modifiable carbon, oxygen, nitrogen or sulfur atom.

In some embodiments, TAMBM is

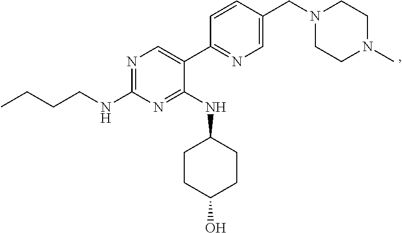 [SMILES: CCCCNc1ncc(-c2ccc(CN3CCN(C)CC3)cn2)c(N[C@H]2CC[C@H](O)CC2)n1]

wherein

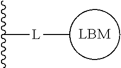

is attached to a modifiable carbon, oxygen, nitrogen or sulfur atom.

In some embodiments, TAMBM is

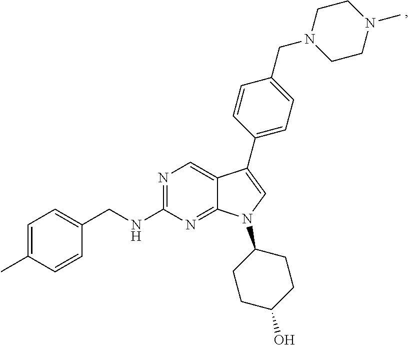

wherein

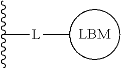

is attached to a modifiable carbon, oxygen, nitrogen or sulfur atom.

In some embodiments, TAMBM is

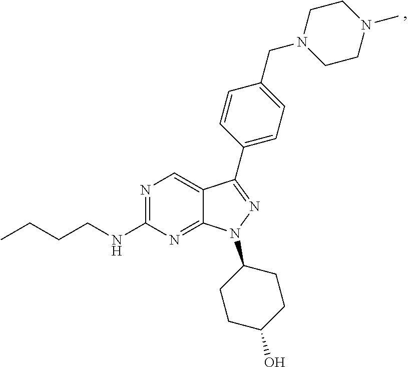 [SMILES: CCCCNc1ncc2c(-c3ccc(CN4CCN(C)CC4)cc3)nn([C@H]3CC[C@H](O)CC3)c2n1]

wherein

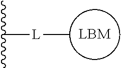

is attached to a modifiable carbon, oxygen, nitrogen or sulfur atom.

In certain embodiments, TAMBM is a pan-TAM binding moiety, binding Tyro3, Axl, and MerTK. In certain embodiments, TAMBM preferentially binds one or two TAM receptor kinases. In some embodiments, TAMBM preferentially binds Tyro3 and Axl relative to MerTK. In some embodiments, TAMBM preferentially binds Tyro3 and MerTK relative to Axl. In some embodiments, TAMBM preferentially binds MerTK and Axl relative to Tyro3. In some embodiments, TAMBM preferentially binds one TAM receptor kinase. In some embodiments, TAMBM is a Tyro3 binding moiety (TBM). In some embodiments, TAMBM is an Axl binding moiety (ABM). In some embodiments, TAMBM is a MerTK binding moiety (MBM).

In some embodiments, TAMBM is selected from those depicted in Table 1, below.

Linker (L)

As defined above and described herein, L is a bivalent moiety that connects TAMBM to LBM or TAMBM to DIM.

In some embodiments, L is a bivalent moiety that connects TAMBM to LBM. In some embodiments, L is a bivalent moiety that connects TAMBM to DIM. In some embodiments, L is a bivalent moiety that connects TAMBM to a lysine mimetic.

In some embodiments, L is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—,

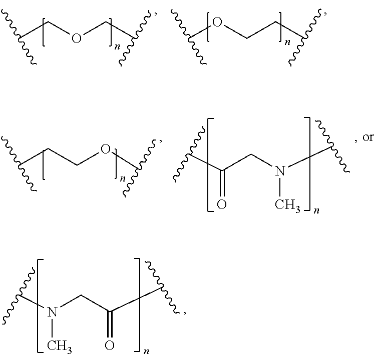

wherein: each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; and wherein n is 0, 1, 2, 3, 4, 5, 6, 7, 8, 9, or 10.

In some embodiments, each -Cy- is independently an optionally substituted bivalent phenylenyl. In some embodiments, each -Cy- is independently an optionally substituted 8-10 membered bicyclic arylenyl. In some embodiments, each -Cy- is independently an optionally substituted 4-7 membered saturated or partially unsaturated carbocyclylenyl. In some embodiments, each -Cy- is independently an optionally substituted 4-7 membered saturated or partially unsaturated spiro carbocyclylenyl. In some embodiments, each -Cy- is independently an optionally substituted 8-10 membered bicyclic saturated or partially unsaturated carbocyclylenyl. In some embodiments, each -Cy- is independently an optionally substituted 4-7 membered saturated or partially unsaturated heterocyclylenyl having 1-2 heteroatoms independently selected from nitrogen, oxygen, and sulfur. In some embodiments, each -Cy- is independently an optionally substituted 4-7 membered saturated or partially unsaturated spiro heterocyclylenyl having 1-2 heteroatoms independently selected from nitrogen, oxygen, and sulfur. In some embodiments, each -Cy- is independently an optionally substituted 8-10 membered bicyclic saturated or partially unsaturated heterocyclylenyl having 1-2 heteroatoms independently selected from nitrogen, oxygen, and sulfur. In some embodiments, each -Cy- is independently an optionally substituted 5-6 membered heteroarylenyl having 1-4 heteroatoms independently selected from nitrogen, oxygen, and sulfur. In some embodiments, each -Cy- is independently an optionally substituted 8-10 membered bicyclic heteroarylenyl having 1-5 heteroatoms independently selected from nitrogen, oxygen, or sulfur.

In some embodiments, -Cy- is

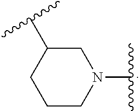 [SMILES: *C1CCCN(*)C1 ; "."]

In some embodiments, -Cy- is

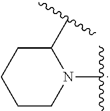 [SMILES: *C1CCCCN1* ; "."]

In some embodiments, -Cy- is

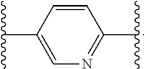 [SMILES: *c1ccc(*)nc1 ; "."]

In some embodiments, -Cy- is

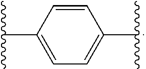 [SMILES: *c1ccc(*)cc1 ; "."]

In some embodiments, -Cy- is

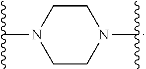 [SMILES: *N1CCN(*)CC1 ; "."]

In some embodiments, -Cy- is

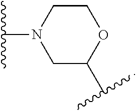 [SMILES: *C1CN(*)CCO1]

In some embodiments, -Cy- is

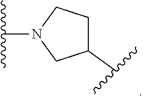 [SMILES: *C1CCN(*)C1]

In some embodiments, -Cy- is

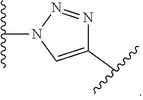 [SMILES: *c1cn(*)nn1]

In some embodiments, -Cy- is

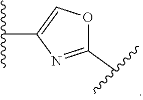 [SMILES: *c1coc(*)n1]

In some embodiments, -Cy- is

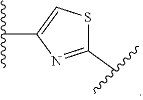 [SMILES: *c1csc(*)n1]

In some embodiments, -Cy- is

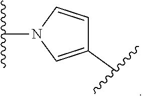 [SMILES: *c1ccn(*)c1]

In some embodiments, -Cy- is

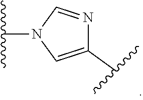 [SMILES: *c1cn(*)cn1]

In some embodiments, -Cy- is

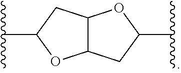 [SMILES: *C1CC2OC(*)CC2O1]

In some embodiments, -Cy- is

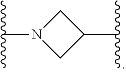 [SMILES: *C1CN(*)C1]

In some embodiments, -Cy- is

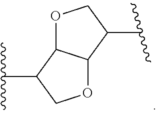 [SMILES: *C1COC2C(*)COC12]

In some embodiments, -Cy- is

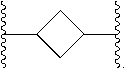 [SMILES: *C1CC(*)C1]

In some embodiments, -Cy- is

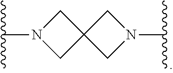 [SMILES: *N1CC2(C1)CN(*)C2]

In some embodiments, -Cy- is

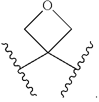 [SMILES: *C1(*)COC1]

In some embodiments, -Cy- is

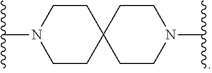 [SMILES: *N1CCC2(CC1)CCN(*)CC2]

In some embodiments, -Cy- is

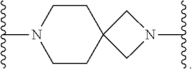 [SMILES: *N1CCC2(CC1)CN(*)C2]

In some embodiments, -Cy- is

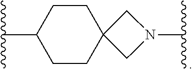 [SMILES: *C1CCC2(CC1)CN(*)C2]

In some embodiments, -Cy- is

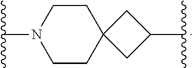 [SMILES: *C1CC2(CCN(*)CC2)C1]

In some embodiments, -Cy- is

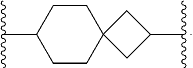 [SMILES: *C1CCC2(CC1)CC(*)C2]

In some embodiments, -Cy- is

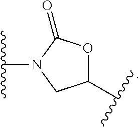 [SMILES: *C1CN(*)C(=O)O1]

In some embodiments, -Cy- is

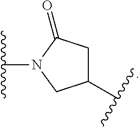 [SMILES: *C1CC(=O)N(*)C1]

In some embodiments, -Cy- is

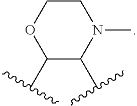 [SMILES: *C1OCCN(C)C1*]

In some embodiments, -Cy- is

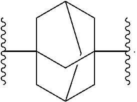 [SMILES: *C12CC3CC(C1)CC(*)(C3)C2]

In some embodiments, -Cy- is selected from those depicted in Table 1, below. In some embodiments, is

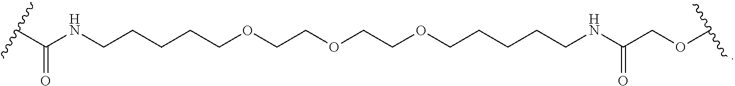 [SMILES: *OCC(=O)NCCCCCOCCOCCOCCCCCNC(*)=O]

In some embodiments, L is

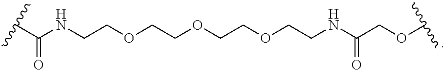 [SMILES: *OCC(=O)NCCOCCOCCOCCNC(*)=O]

In some embodimentsn, L is

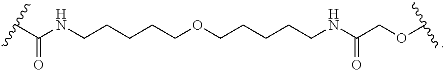 [SMILES: *OCC(=O)NCCCCCOCCCCCNC(*)=O]

In some embodiments, L is

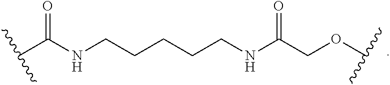 [SMILES: *OCC(=O)NCCCCCNC(*)=O]

In some embodiments, L is

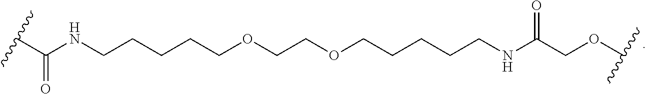 [SMILES: *OCC(=O)NCCCCCOCCOCCCCCNC(*)=O]

In some embodiments, L is

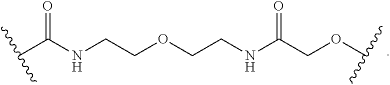 [SMILES: *OCC(=O)NCCOCCNC(*)=O]

In some embodiments, L is

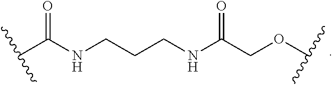 [SMILES: *OCC(=O)NCCCNC(*)=O]

In some embodiments, L is

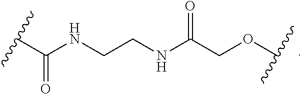 [SMILES: *OCC(=O)NCCNC(*)=O]

In some embodiments, L is

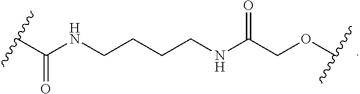 [SMILES: *OCC(=O)NCCCCNC(*)=O]

In some embodiments, L is

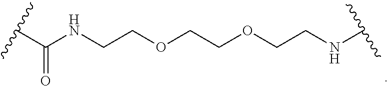 [SMILES: *NCCOCCOCCNC(*)=O]

In some embodiments, L is

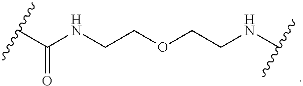 [SMILES: *NCCOCCNC(*)=O]

In some embodiments, L is

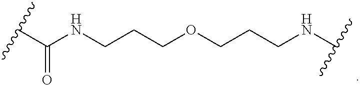 [SMILES: *NCCCOCCCNC(*)=O]

In some embodiments, L is

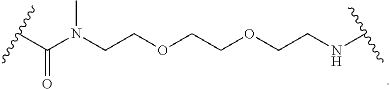 [SMILES: *NCCOCCOCCN(C)C(*)=O]

In some embodiments, L is

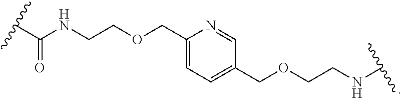 [SMILES: *NCCOCc1ccc(COCCNC(*)=O)nc1]

In some embodiments, L is

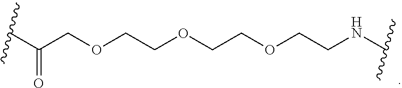 [SMILES: *NCCOCCOCCOCC(*)=O]

In some embodiments, L is

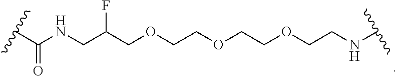 [SMILES: *NCCOCCOCCOCC(F)CNC(*)=O]

In some embodiments, L is

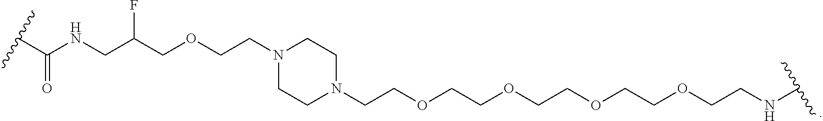 [SMILES: *NCCOCCOCCOCCOCCN1CCN(CCOCC(F)CNC(*)=O)CC1]

In some embodiments, L is

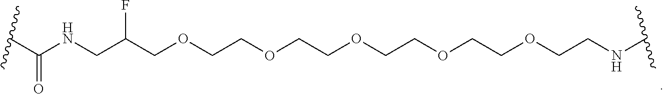 [SMILES: *NCCOCCOCCOCCOCCOCC(F)CNC(*)=O]

In some embodiments, L is

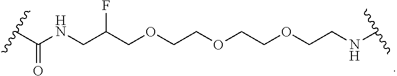 [SMILES: *NCCOCCOCCOCC(F)CNC(*)=O]

In some embodiments, L is

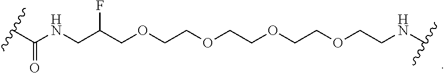 [SMILES: *NCCOCCOCCOCCOCC(F)CNC(*)=O]

In some embodiments, L is

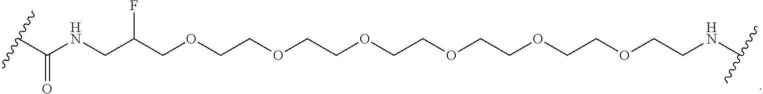 [SMILES: *NCCOCCOCCOCCOCCOCCOCC(F)CNC(*)=O]

In some embodiments, L is

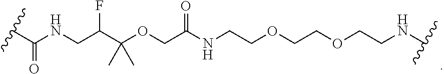 [SMILES: *NCCOCCOCCNC(=O)COC(C)(C)C(F)CNC(*)=O]

In some embodiments, L is

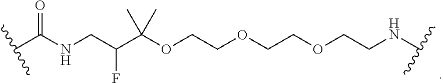 [SMILES: *NCCOCCOCCOC(C)(C)C(F)CNC(*)=O]

In some embodiments, L is

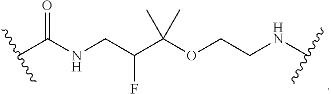 [SMILES: *NCCOC(C)(C)C(F)CNC(*)=O]

In some embodiments, L is

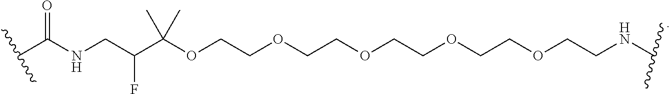 [SMILES: *NCCOCCOCCOCCOCCOC(C)(C)C(F)CNC(*)=O]

In some embodiments, L is

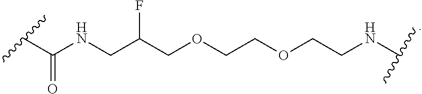 [SMILES: *NCCOCCOCC(F)CNC(*)=O]

In some embodiments, L is

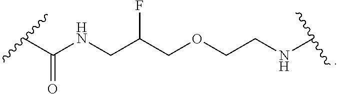 [SMILES: *NCCOCC(F)CNC(*)=O]

In some embodiments, L is

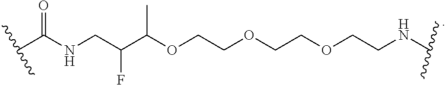 [SMILES: *NCCOCCOCCOC(C)C(F)CNC(*)=O]

In some embodiments, L is

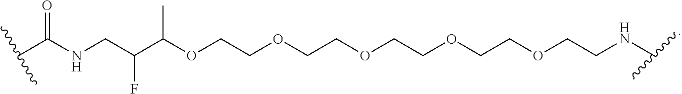 [SMILES: *NCCOCCOCCOCCOCCOC(C)C(F)CNC(*)=O]

In some embodiments, L is

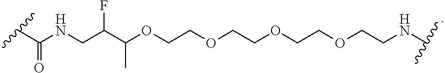 [SMILES: *NCCOCCOCCOCCOC(C)C(F)CNC(*)=O]

In some embodiments, L is

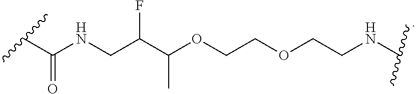 [SMILES: *NCCOCCOC(C)C(F)CNC(*)=O]

In some embodiments, L is

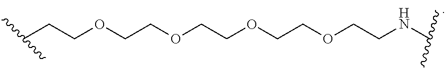 [SMILES: *CCOCCOCCOCCOCCN*]

In some embodiments, L is

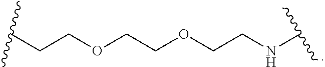 [SMILES: *CCOCCOCCN*]

In some embodiments, L is

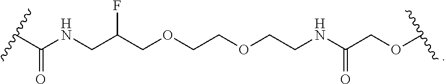 [SMILES: *OCC(=O)NCCOCCOCC(F)CNC(*)=O]

In some embodiments, L is

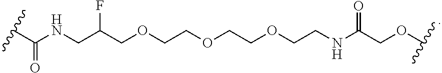 [SMILES: *OCC(=O)NCCOCCOCCOCC(F)CNC(*)=O]

In some embodiments, L is

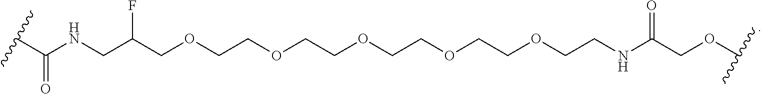 [SMILES: *OCC(=O)NCCOCCOCCOCCOCCOCC(F)CNC(*)=O]

In some embodiments, L is

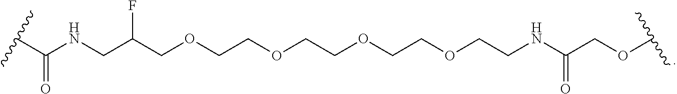 [SMILES: *OCC(=O)NCCOCCOCCOCCOCC(F)CNC(*)=O]

In some embodiments, L is

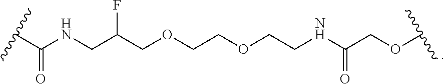 [SMILES: *OCC(=O)NCCOCCOCC(F)CNC(*)=O]

In some embodiments, L is

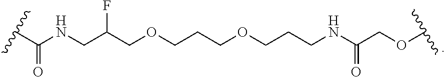 [SMILES: *OCC(=O)NCCCOCCCOCC(F)CNC(*)=O]

In some embodiments, L is

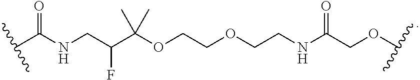 [SMILES: *OCC(=O)NCCOCCOC(C)(C)C(F)CNC(*)=O]

In some embodiments, L is

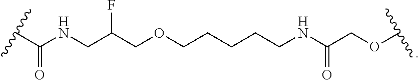 [SMILES: *OCC(=O)NCCCCCOCC(F)CNC(*)=O]

In some embodiments, L is

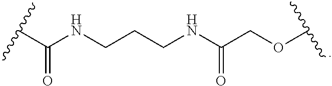 [SMILES: *OCC(=O)NCCCNC(*)=O]

In some embodiments, L is

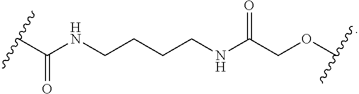 [SMILES: *OCC(=O)NCCCCNC(*)=O]

In some embodiments, L is

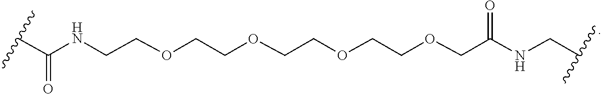 [SMILES: *CNC(=O)COCCOCCOCCOCCNC(*)=O]

In some embodiments, L

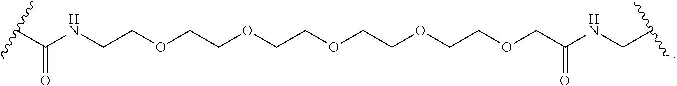 [SMILES: *CNC(=O)COCCOCCOCCOCCOCCNC(*)=O]

is In some embodiments, L is

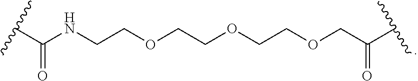 [SMILES: *C(=O)COCCOCCOCCNC(*)=O]

In some embodiments, L is

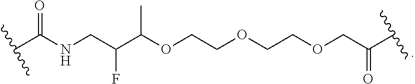 [SMILES: *C(=O)COCCOCCOC(C)C(F)CNC(*)=O]

In some embodiments, L is

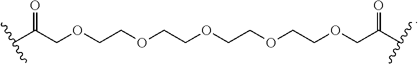 [SMILES: *C(=O)COCCOCCOCCOCCOCC(*)=O]

In some embodiments, L is

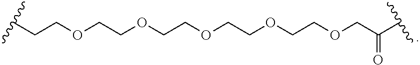 [SMILES: *CCOCCOCCOCCOCCOCC(*)=O]

In some embodiments, L is

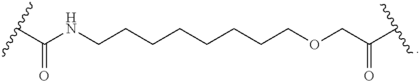 [SMILES: *C(=O)COCCCCCCCCNC(*)=O]

In some embodiments, L is

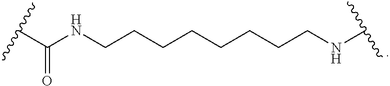 [SMILES: *NCCCCCCCCNC(*)=O]

In some embodiments, L is

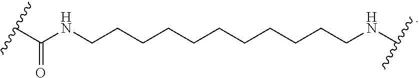 [SMILES: *NCCCCCCCCCCCNC(*)=O]

In some embodiments, L is

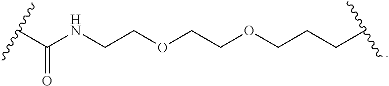 [SMILES: *CCCOCCOCCNC(*)=O]

In some embodiments, L is

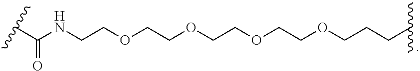 [SMILES: *CCCOCCOCCOCCOCCNC(*)=O]

In some embodiments, L is

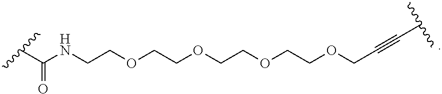 [SMILES: *C#CCOCCOCCOCCOCCNC(*)=O]

In some embodiments, L is

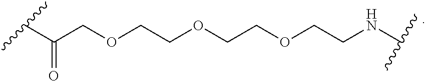 [SMILES: *NCCOCCOCCOCC(*)=O]

In some embodiments, L is

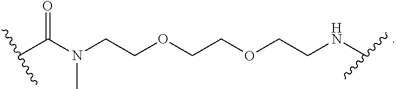 [SMILES: *NCCOCCOCCN(C)C(*)=O]

In some embodiments, L is

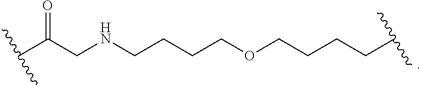 [SMILES: *CCCCOCCCCNCC(*)=O]

In some embodiments, L is

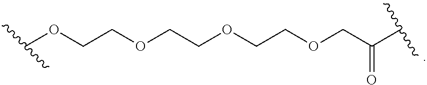 [SMILES: *OCCOCCOCCOCC(*)=O]

In some embodiments, L is

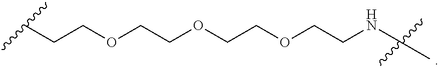 [SMILES: *CCOCCOCCOCCN*]

In some embodiments, L is

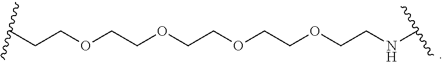 [SMILES: *CCOCCOCCOCCOCCN*]

In some embodiments, L is

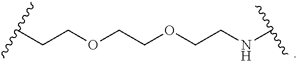 [SMILES: *CCOCCOCCN*]

In some embodiments, L is

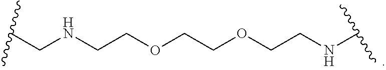 [SMILES: *CNCCOCCOCCN*]

In some embodiments, L is

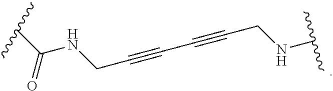 [SMILES: *NCC#CC#CCNC(*)=O]

In some embodiments, L is

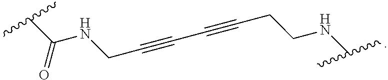 [SMILES: *NCCC#CC#CCNC(*)=O]

In some embodiments, L is

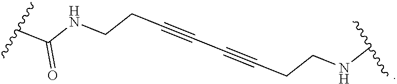 [SMILES: *NCCC#CC#CCCNC(*)=O]

In some embodiments, L is

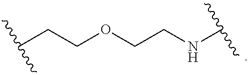 [SMILES: *CCOCCN*]

In some embodiments, L is

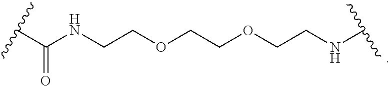 [SMILES: *NCCOCCOCCNC(*)=O]

In some embodiments, L is

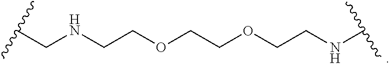 [SMILES: *CNCCOCCOCCN*]

In some embodiments, L is

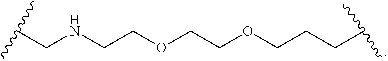 [SMILES: *CCCOCCOCCNC*]

In some embodiments, L is

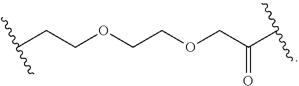 [SMILES: *CCOCCOCC(*)=O]

In some embodiments, L is

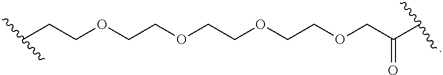 [SMILES: *CCOCCOCCOCCOCC(*)=O]

In some embodiments, L is

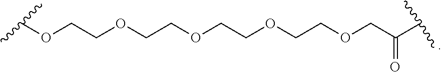 [SMILES: *OCCOCCOCCOCCOCC(*)=O]

In some embodiments, L is

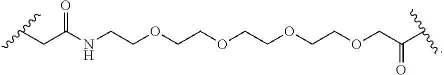 [SMILES: *CC(=O)NCCOCCOCCOCCOCC(*)=O]

In some embodiments, L is

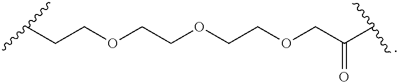 [SMILES: *CCOCCOCCOCC(*)=O]

In some embodiments, L is

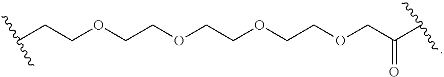 [SMILES: *CCOCCOCCOCCOCC(*)=O]

In some embodiments, L is

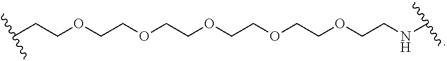 [SMILES: *CCOCCOCCOCCOCCOCCN*]

In some embodiments, L is

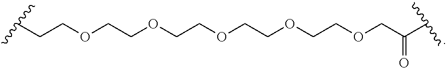 [SMILES: *CCOCCOCCOCCOCCOCC(*)=O]

In some embodiments, L is

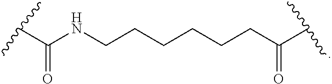 [SMILES: *C(=O)CCCCCCNC(*)=O]

In some embodiments, L is

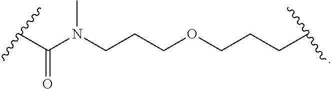 [SMILES: *CCCOCCCN(C)C(*)=O]

In some embodiments, L is

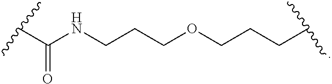 [SMILES: *CCCOCCCNC(*)=O]

In some embodiments, L is

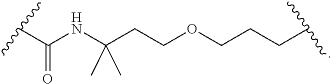 [SMILES: *CCCOCCC(C)(C)NC(*)=O]

In some embodiments, L is

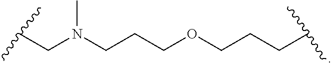 [SMILES: *CCCOCCCN(C)C*]

In some embodiments, L is

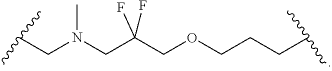 [SMILES: *CCCOCC(F)(F)CN(C)C*]

In some embodiments, L is

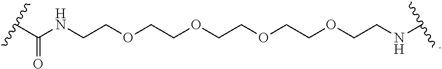 [SMILES: *NCCOCCOCCOCCOCCNC(*)=O]

In some embodiments, L is

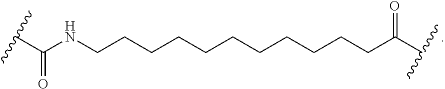 [SMILES: *C(=O)CCCCCCCCCCCNC(*)=O]

In some embodiments, L is

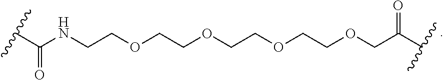 [SMILES: *C(=O)COCCOCCOCCOCCNC(*)=O]

In some embodiments, L is

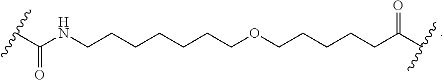 [SMILES: *C(=O)CCCCCOCCCCCCCNC(*)=O]

In some embodiments, L is

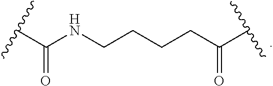 [SMILES: *C(=O)CCCCNC(*)=O]

In some embodiments, L is

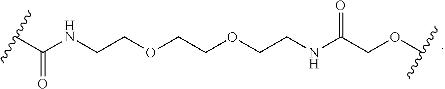 [SMILES: *OCC(=O)NCCOCCOCCNC(*)=O]

In some embodiments, L is

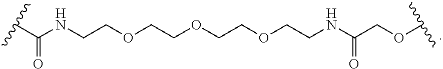 [SMILES: *OCC(=O)NCCOCCOCCOCCNC(*)=O]

In some embodiments, L is

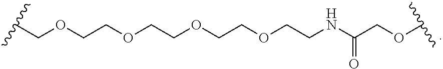 [SMILES: *COCCOCCOCCOCCNC(=O)CO*]

In some embodiments, L is

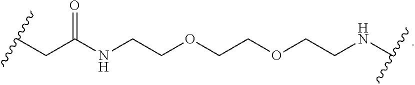 [SMILES: *CC(=O)NCCOCCOCCN*]

In some embodiments, L is

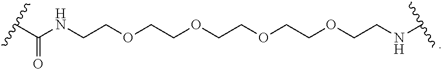 [SMILES: *NCCOCCOCCOCCOCCNC(*)=O]

In some embodiments, L is

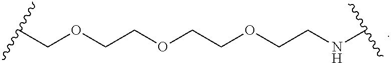 [SMILES: *COCCOCCOCCN*]

In some embodiments, L is

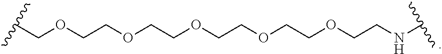 [SMILES: *COCCOCCOCCOCCOCCN*]

In some embodiments, L is

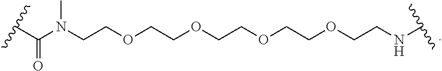 [SMILES: *NCCOCCOCCOCCOCCN(C)C(*)=O]

In some embodiments, L is

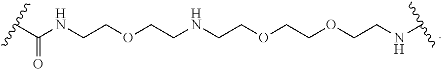 [SMILES: *NCCOCCOCCNCCOCCNC(*)=O]

In some embodiments, L is

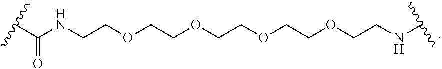 [SMILES: *NCCOCCOCCOCCOCCNC(*)=O]

In some embodiments, L is

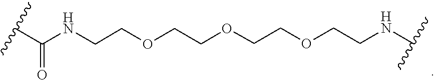 [SMILES: *NCCOCCOCCOCCNC(*)=O]

In some embodiments, L is

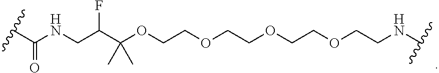 [SMILES: *NCCOCCOCCOCCOC(C)(C)C(F)CNC(*)=O]

In some embodiments, L is

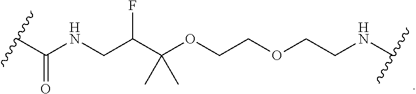 [SMILES: *NCCOCCOC(C)(C)C(F)CNC(*)=O]

In some embodiments, L is

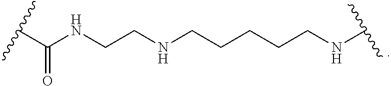 [SMILES: *NCCCCCNCCNC(*)=O]

In some embodiments, L is

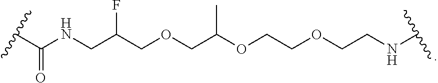 [SMILES: *NCCOCCOC(C)COCC(F)CNC(*)=O]

In some embodiments, L is

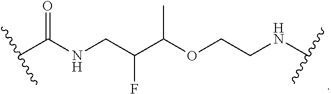 [SMILES: *NCCOC(C)C(F)CNC(*)=O]

In some embodiments, L is

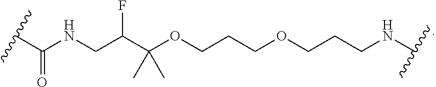 [SMILES: *NCCCOCCCOC(C)(C)C(F)CNC(*)=O]

In some embodiments, L is

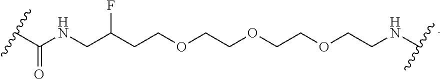 [SMILES: *NCCOCCOCCOCCC(F)CNC(*)=O]

In some embodiments, L is

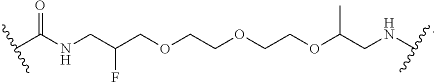 [SMILES: *NCC(C)OCCOCCOCC(F)CNC(*)=O]

In some embodiments, L is

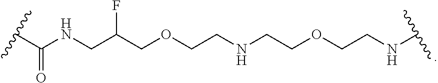 [SMILES: *NCCOCCNCCOCC(F)CNC(*)=O]

In some embodiments, L is

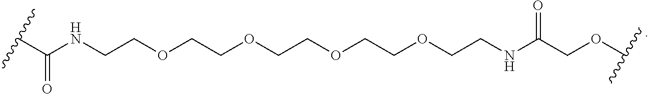 [SMILES: *OCC(=O)NCCOCCOCCOCCOCCNC(*)=O]

In some embodiments, L is

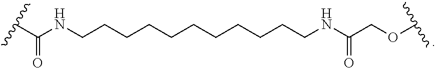 [SMILES: *OCC(=O)NCCCCCCCCCCCNC(*)=O]

In some embodiments, L is

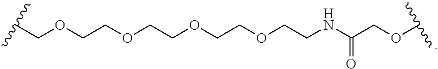 [SMILES: *COCCOCCOCCOCCNC(=O)CO*]

In some embodiments, L is

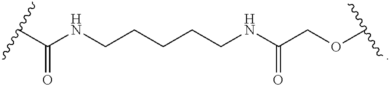 [SMILES: *OCC(=O)NCCCCCNC(*)=O]

In some embodiments, L is

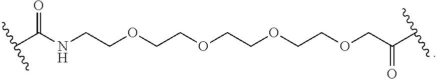 [SMILES: *C(=O)COCCOCCOCCOCCNC(*)=O]

In some embodiments, L is

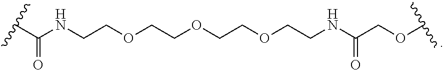 [SMILES: *OCC(=O)NCCOCCOCCOCCNC(*)=O]

In some embodiments, L is

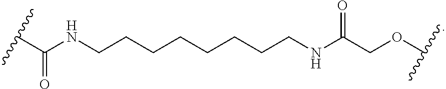 [SMILES: *OCC(=O)NCCCCCCCCNC(*)=O]

In some embodiments, L is

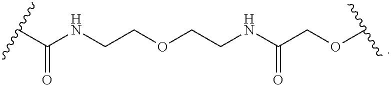 [SMILES: *OCC(=O)NCCOCCNC(*)=O]

In some embodiments, L is

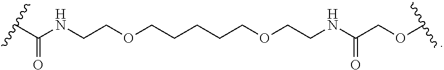 [SMILES: *OCC(=O)NCCOCCCCCOCCNC(*)=O]

In some embodiments, L is

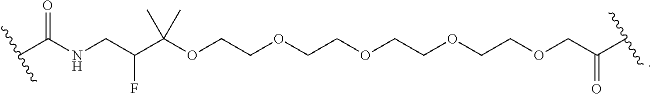 [SMILES: *C(=O)COCCOCCOCCOCCOC(C)(C)C(F)CNC(*)=O]

In some embodiments, L is

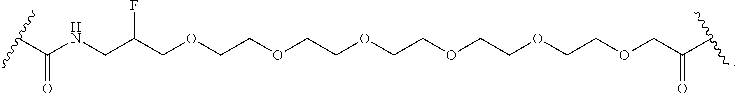 [SMILES: *C(=O)COCCOCCOCCOCCOCCOCC(F)CNC(*)=O]

In some embodiments, L is

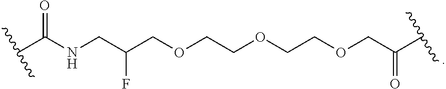 [SMILES: *C(=O)COCCOCCOCC(F)CNC(*)=O]

In some embodiments, L is

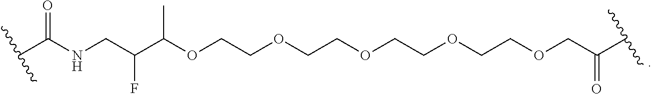 [SMILES: *C(=O)COCCOCCOCCOCCOC(C)C(F)CNC(*)=O]

In some embodiments, L is

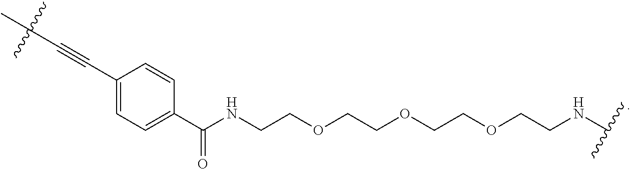 [SMILES: *C#Cc1ccc(C(=O)NCCOCCOCCOCCN*)cc1]

In some embodiments, L is

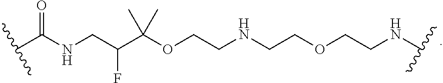 [SMILES: *NCCOCCNCCOC(C)(C)C(F)CNC(*)=O]

In some embodiments, L is

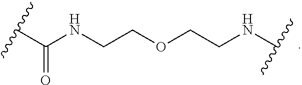 [SMILES: *NCCOCCNC(*)=O]

In some embodiments, L is

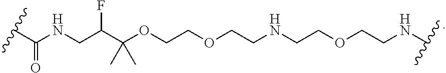 [SMILES: *NCCOCCNCCOCCOC(C)(C)C(F)CNC(*)=O]

In some embodiments, L is

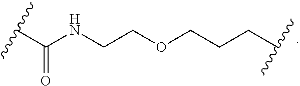 [SMILES: *CCCOCCNC(*)=O]

In some embodiments, L is

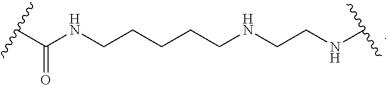 [SMILES: *NCCNCCCCCNC(*)=O]

In some embodiments, L is

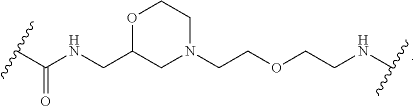 [SMILES: *NCCOCCN1CCOC(CNC(*)=O)C1]

In some embodiments, L is

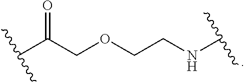 [SMILES: *NCCOCC(*)=O]

In some embodiments, L is

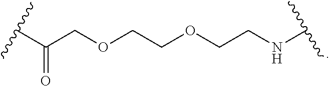 [SMILES: *NCCOCCOCC(*)=O]

In some embodiments, L is

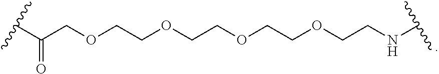 [SMILES: *NCCOCCOCCOCCOCC(*)=O]

In some embodiments, L is

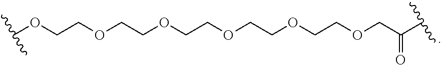 [SMILES: *OCCOCCOCCOCCOCCOCC(*)=O]

In some embodiments, L is

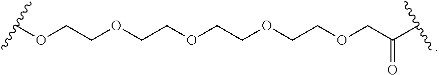 [SMILES: *OCCOCCOCCOCCOCC(*)=O]

In some embodiments, L is

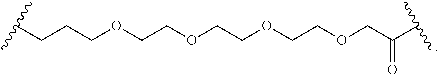 [SMILES: *CCCOCCOCCOCCOCC(*)=O]

In some embodiments, L is

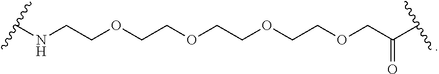 [SMILES: *NCCOCCOCCOCCOCC(*)=O]

In some embodiments, L is

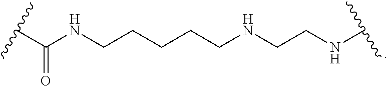 [SMILES: *NCCNCCCCCNC(*)=O]

In some embodiments, L is

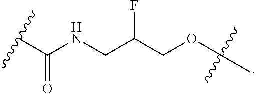 [SMILES: *OCC(F)CNC(*)=O]

In some embodiments, L is

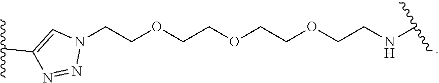 [SMILES: *NCCOCCOCCOCCn1cc(*)nn1]

In some embodiments, L is

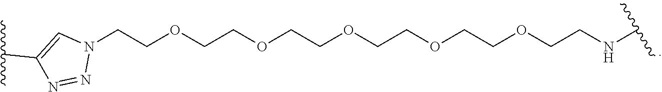 [SMILES: *NCCOCCOCCOCCOCCOCCn1cc(*)nn1]

In some embodiments, L is

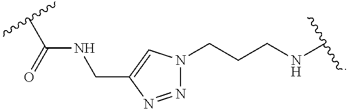 [SMILES: *NCCCn1cc(CNC(*)=O)nn1]

In some embodiments, L

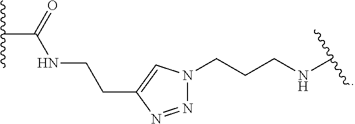 [SMILES: *NCCCn1cc(CCNC(*)=O)nn1]

In some embodiments, L is

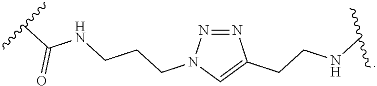 [SMILES: *NCCc1cn(CCCNC(*)=O)nn1]

In some embodiments, L is

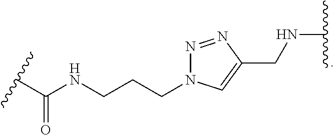 [SMILES: *NCc1cn(CCCNC(*)=O)nn1]

In some embodiments, L is

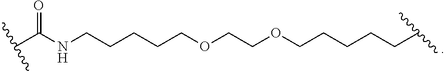 [SMILES: *CCCCCOCCOCCCCCNC(*)=O]

In some embodiments, L is

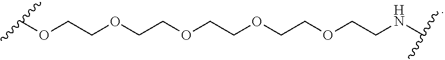 [SMILES: *NCCOCCOCCOCCOCCO*]

In some embodiments, L is

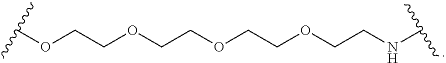 [SMILES: *NCCOCCOCCOCCO*]

In some embodiments, L is

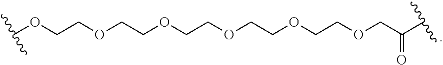 [SMILES: *OCCOCCOCCOCCOCCOCC(*)=O]

In some embodiments, L is

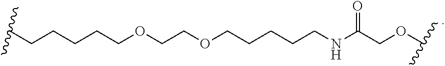 [SMILES: *CCCCCOCCOCCCCCNC(=O)CO*]

In some embodiments, L is

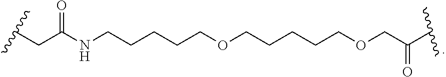 [SMILES: *CC(=O)NCCCCCOCCCCCOCC(*)=O]

In some embodiments, L is

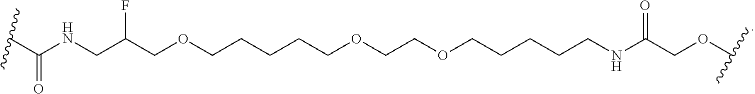 [SMILES: *OCC(=O)NCCCCCOCCOCCCCCOCC(F)CNC(*)=O]

In some embodiments, L is

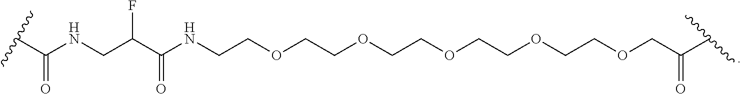 [SMILES: *C(=O)COCCOCCOCCOCCOCCNC(=O)C(F)CNC(*)=O]

In some embodiments, L is

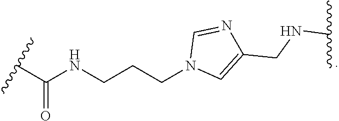 [SMILES: *NCc1cn(CCCNC(*)=O)cn1]

In some embodiments, L is

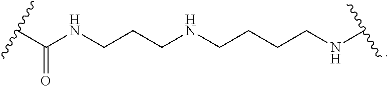 [SMILES: *NCCCCNCCCNC(*)=O]

In some embodiments, L is

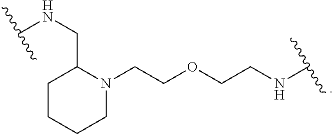 [SMILES: *NCCOCCN1CCCCC1CN*]

In some embodiments, L is

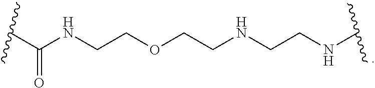 [SMILES: *NCCNCCOCCNC(*)=O]

In some embodiments, L is

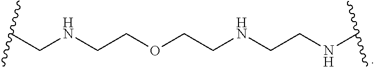 [SMILES: *CNCCOCCNCCN*]

In some embodiment, L is

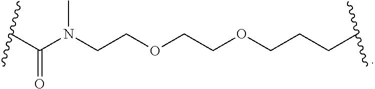 [SMILES: *CCCOCCOCCN(C)C(*)=O]

In some embodiment, L is

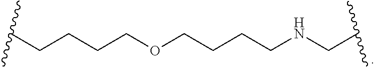 [SMILES: *CCCCOCCCCNC*]

In some embodiment, L is

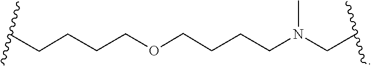 [SMILES: *CCCCOCCCCN(C)C*]

In some embodiments, L is

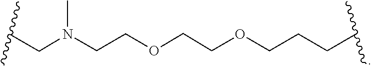 [SMILES: *CCCOCCOCCN(C)C*]

In some embodiments, L is

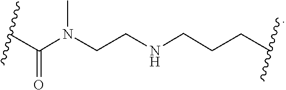 [SMILES: *CCCNCCN(C)C(*)=O]

In some embodiments, L is

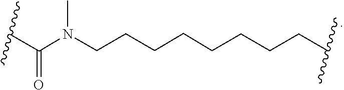 [SMILES: *CCCCCCCCN(C)C(*)=O]

In some embodiments, L is

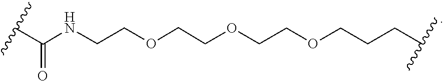 [SMILES: *CCCOCCOCCOCCNC(*)=O]

In some embodiments, L is

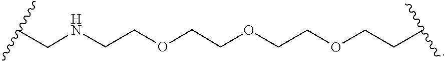 [SMILES: *CCOCCOCCOCCNC*]

In some embodiments, L is

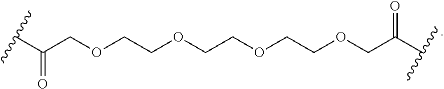 [SMILES: *C(=O)COCCOCCOCCOCC(*)=O]

In some embodiments, L is

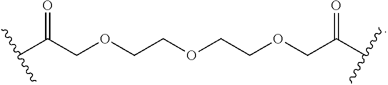 [SMILES: *C(=O)COCCOCCOCC(*)=O]

In In some embodiments, L is

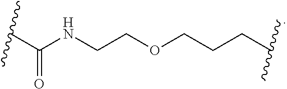 [SMILES: *CCCOCCNC(*)=O]

In some embodiments, L is

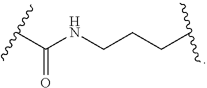 [SMILES: *CCCNC(*)=O]

In some embodiments, L is

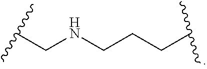 [SMILES: *CCCNC*]

In some embodiments, L is

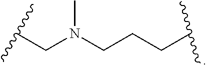 [SMILES: *CCCN(C)C*]

In some embodiments, L is

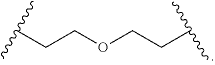 [SMILES: *CCOCC*]

In some embodiments, L is

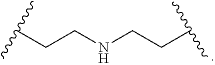 [SMILES: *CCNCC*]

In some embodiments, L is

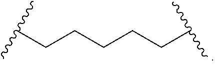 [SMILES: *CCCCC*]

In some embodiments, L is

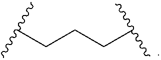 [SMILES: *CCC*]

In some embodiments, L is

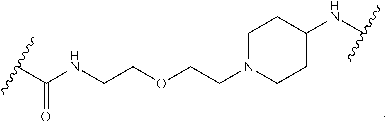 [SMILES: *NC1CCN(CCOCCNC(*)=O)CC1]

In some embodiments, L is

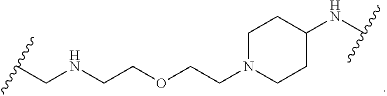 [SMILES: *CNCCOCCN1CCC(N*)CC1]

In some embodiments, L is

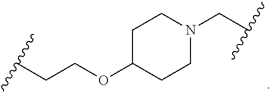 [SMILES: *CCOC1CCN(C*)CC1]

In some embodiments, L is

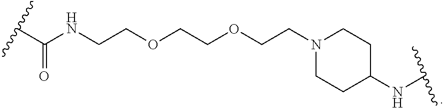 [SMILES: *NC1CCN(CCOCCOCCNC(*)=O)CC1]

In some embodiments, L is

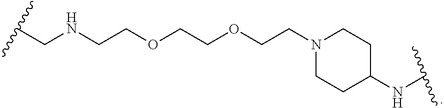 [SMILES: *CNCCOCCOCCN1CCC(N*)CC1]

In some embodiments, L is

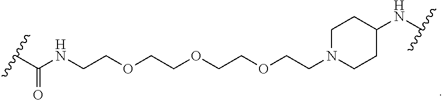 [SMILES: *NC1CCN(CCOCCOCCOCCNC(*)=O)CC1]

In some embodiments, L is

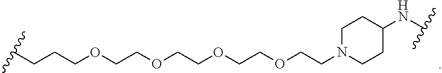 [SMILES: *CCCOCCOCCOCCOCCN1CCC(N*)CC1]

In some embodiments, L is

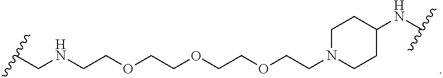 [SMILES: *CNCCOCCOCCOCCN1CCC(N*)CC1]

In some embodiments, L is

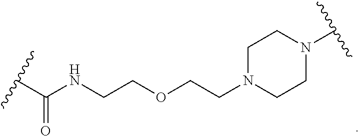 [SMILES: *C(=O)NCCOCCN1CCN(*)CC1]

In some embodiments, L is

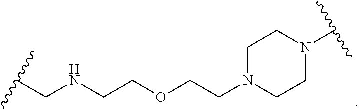 [SMILES: *CNCCOCCN1CCN(*)CC1]

In some embodiments, L is

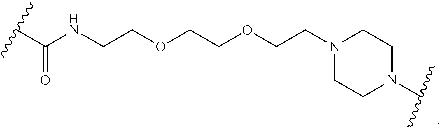 [SMILES: *C(=O)NCCOCCOCCN1CCN(*)CC1]

In some embodiments, L is

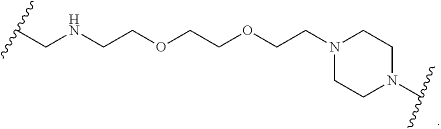 [SMILES: *CNCCOCCOCCN1CCN(*)CC1]

In some embodiments, L is

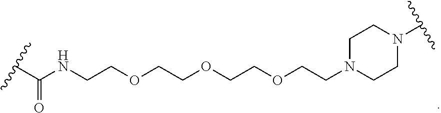 [SMILES: *C(=O)NCCOCCOCCOCCN1CCN(*)CC1]

In some embodiments, L is

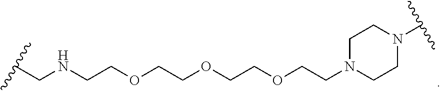 [SMILES: *CNCCOCCOCCOCCN1CCN(*)CC1]

In some embodiments, L is

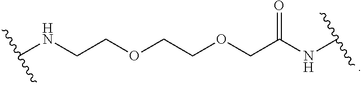 [SMILES: *NCCOCCOCC(=O)N*]

In some embodiments, L is

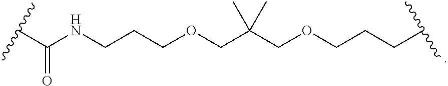 [SMILES: *CCCOCC(C)(C)COCCCNC(*)=O]

In some embodiments, L is

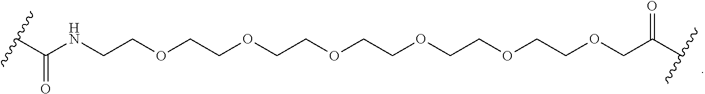 [SMILES: *C(=O)COCCOCCOCCOCCOCCOCCNC(*)=O]

In some embodiments, L is

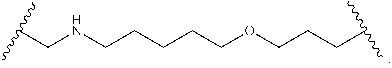 [SMILES: *CCCOCCCCCNC*]

In some embodiments, L is

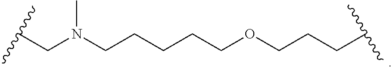 [SMILES: *CCCOCCCCCN(C)C*]

In some embodiments, L is

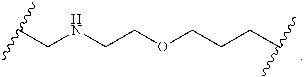 [SMILES: *CCCOCCNC*]

In some embodiments, L is

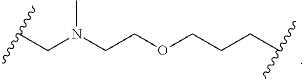 [SMILES: *CCCOCCN(C)C*]

In some embodiments, L is

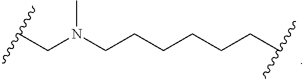 [SMILES: *CCCCCCN(C)C*]

In some embodiments, L is

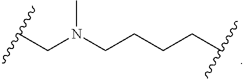 [SMILES: *CCCCN(C)C*]

In some embodiments, L is

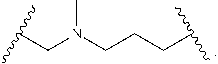 [SMILES: *CCCN(C)C*]

In some embodiments, L is

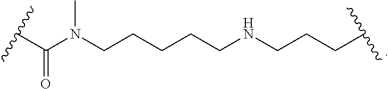 [SMILES: *CCCNCCCCCN(C)C(*)=O]

In some embodiments, L is

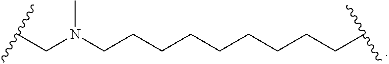 [SMILES: *CCCCCCCCCN(C)C*]

In some embodiments, L is

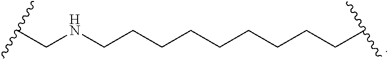 [SMILES: *CCCCCCCCCNC*]

In some embodiments, L is

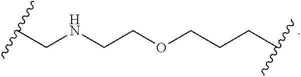 [SMILES: *CCCOCCNC*]

In some embodiments, L is

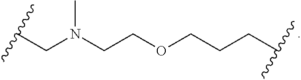 [SMILES: *CCCOCCN(C)C*]

In some embodiments, L is

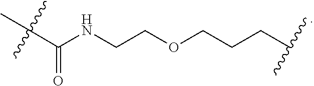 [SMILES: *CCCOCCNC(*)=O]

In some embodiments, L is

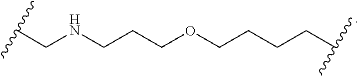 [SMILES: *CCCCOCCCNC*]

In some embodiments, L is

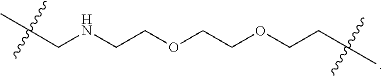 [SMILES: *CCOCCOCCNC*]

In some embodiments, L is

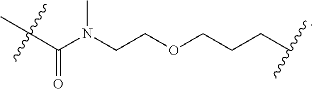 [SMILES: *CCCOCCN(C)C(*)=O]

In some embodiments, L is

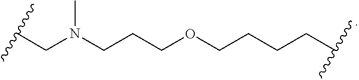 [SMILES: *CCCCOCCCN(C)C*]

In some embodiments, L is

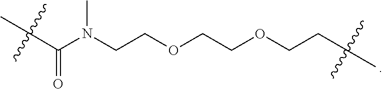 [SMILES: *CCOCCOCCN(C)C(*)=O]

In some embodiments, L is

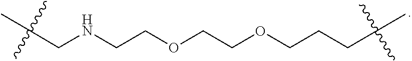 [SMILES: *CCCOCCOCCNC*]

In some embodiments, L is

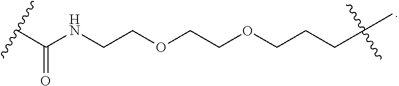 [SMILES: *CCCOCCOCCNC(*)=O]

In some embodiments, L is

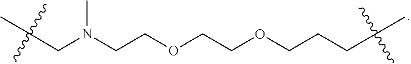 [SMILES: *CCCOCCOCCN(C)C*]

In some embodiments, L is

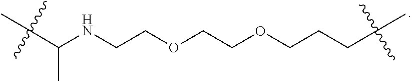 [SMILES: *CCCOCCOCCNC(*)C]

In some embodiments, L is

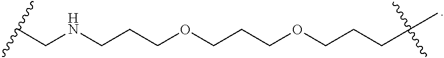 [SMILES: *CCCOCCCOCCCNC*]

In some embodiment, L is

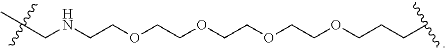 [SMILES: *CCCOCCOCCOCCOCCNC*]

In some embodiment, L is

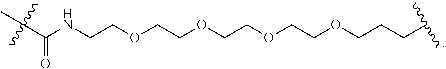 [SMILES: *CCCOCCOCCOCCOCCNC(*)=O]

In some embodiments, L is

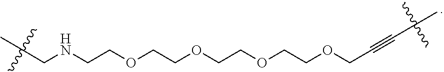 [SMILES: *C#CCOCCOCCOCCOCCNC*]

In some embodiments, L is

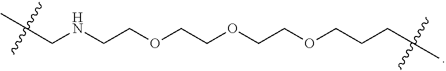 [SMILES: *CCCOCCOCCOCCNC*]

In some embodiments, L is

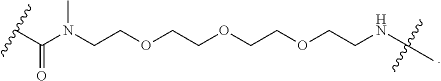 [SMILES: *NCCOCCOCCOCCN(C)C(*)=O]

In some embodiments, L is

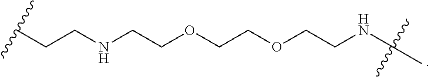 [SMILES: *CCNCCOCCOCCN*]

In some embodiments, L is

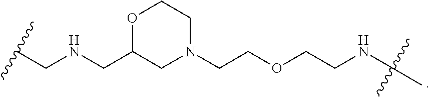 [SMILES: *CNCC1CN(CCOCCN*)CCO1]

In some embodiments, L is

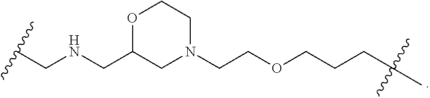 [SMILES: *CCCOCCN1CCOC(CNC*)C1]

In some embodiments, L is

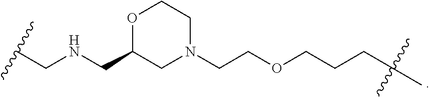 [SMILES: *CCCOCCN1CCO[C@H](CNC*)C1]

In some embodiments, L is

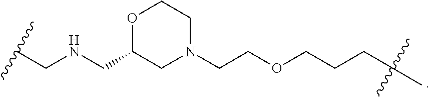 [SMILES: *CCCOCCN1CCO[C@@H](CNC*)C1]

In some embodiments, L is

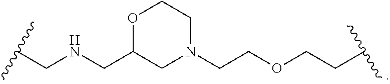 [SMILES: *CCOCCN1CCOC(CNC*)C1]

In some embodiments, L is

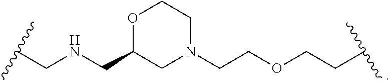 [SMILES: *CCOCCN1CCO[C@H](CNC*)C1]

In some embodiments, L is

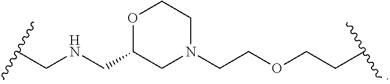 [SMILES: *CCOCCN1CCO[C@@H](CNC*)C1]

In some embodiments, L is

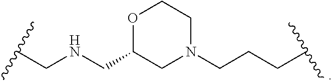 [SMILES: *CCCN1CCO[C@@H](CNC*)C1]

In some embodiments, L is

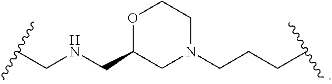 [SMILES: *CCCN1CCO[C@H](CNC*)C1]

In some embodiments, L is

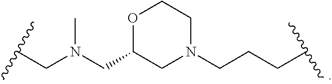 [SMILES: *CCCN1CCO[C@@H](CN(C)C*)C1]

In some embodiments, L is

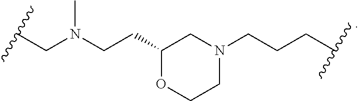 [SMILES: *CCCN1CCO[C@H](CCN(C)C*)C1]

In some embodiments, L

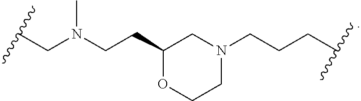 [SMILES: *CCCN1CCO[C@@H](CCN(C)C*)C1]

In some embodiments, L is

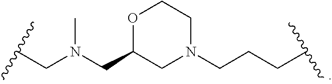 [SMILES: *CCCN1CCO[C@H](CN(C)C*)C1]

In some embodiments, L is

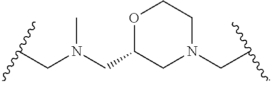 [SMILES: *CN(C)C[C@H]1CN(C*)CCO1]

In some embodiments, L is

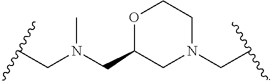 [SMILES: *CN(C)C[C@@H]1CN(C*)CCO1]

In some embodiments, L is

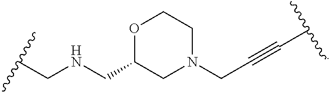

In some embodiments, L is

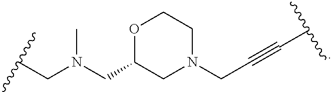

In some embodiments, L is

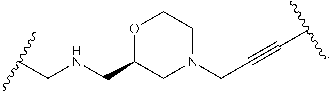

In some embodiments, L is

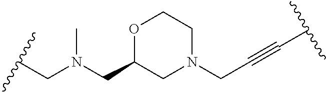

In some embodiments, L is

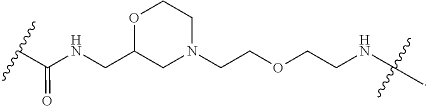 [SMILES: *NCCOCCN1CCOC(CNC(*)=O)C1]

In some embodiments, L is

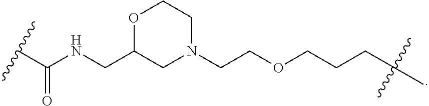 [SMILES: *CCCOCCN1CCOC(CNC(*)=O)C1]

In some embodiments, L is

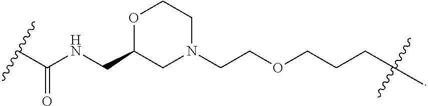 [SMILES: *CCCOCCN1CCO[C@H](CNC(*)=O)C1]

In some embodiments, L is

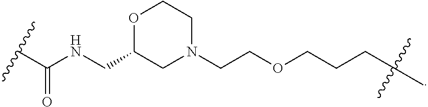 [SMILES: *CCCOCCN1CCO[C@@H](CNC(*)=O)C1]

In some embodiments, L is

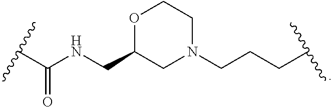 [SMILES: *CCCN1CCO[C@H](CNC(*)=O)C1]

In some embodiments, L is

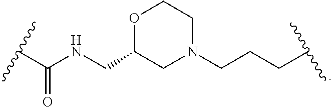 [SMILES: *CCCN1CCO[C@@H](CNC(*)=O)C1]

In some embodiments, L is

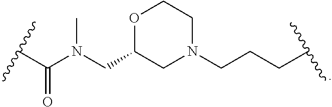 [SMILES: *CCCN1CCO[C@@H](CN(C)C(*)=O)C1]

In some embodiments, L is

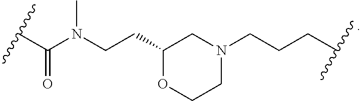 [SMILES: *CCCN1CCO[C@H](CCN(C)C(*)=O)C1]

In some embodiments, L is

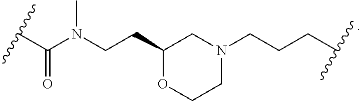 [SMILES: *CCCN1CCO[C@@H](CCN(C)C(*)=O)C1]

In some embodiments, L is

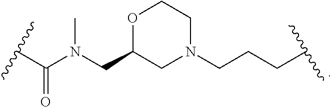 [SMILES: *CCCN1CCO[C@H](CN(C)C(*)=O)C1]

In some embodiments, L is

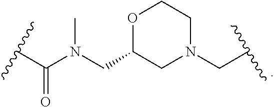 [SMILES: *CN1CCO[C@@H](CN(C)C(*)=O)C1]

In some embodiments, L is

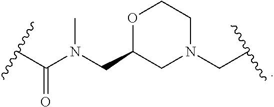 [SMILES: *CN1CCO[C@H](CN(C)C(*)=O)C1]

In some embodiments, L is

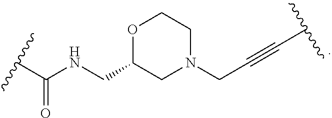 [SMILES: *C#CCN1CCO[C@@H](CNC(*)=O)C1]

In some embodiments, L is

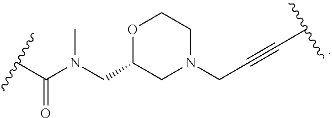 [SMILES: *C#CCN1CCO[C@@H](CN(C)C(*)=O)C1]

In some embodiments, L is

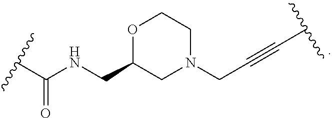 [SMILES: *C#CCN1CCO[C@H](CNC(*)=O)C1]

In some embodiments, L is

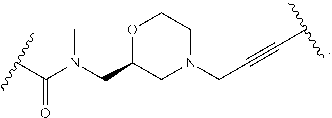 [SMILES: *C#CCN1CCO[C@H](CN(C)C(*)=O)C1]

In some embodiments, L is

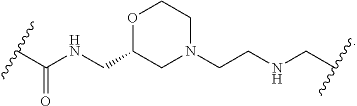 [SMILES: *CNCCN1CCO[C@@H](CNC(*)=O)C1]

In some embodiments, L is

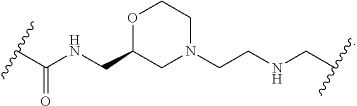 [SMILES: *CNCCN1CCO[C@H](CNC(*)=O)C1]

In some embodiments, L is

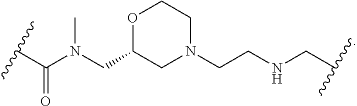 [SMILES: *CNCCN1CCO[C@@H](CN(C)C(*)=O)C1]

In some embodiments, L is

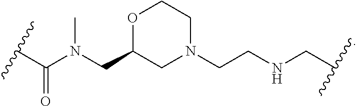 [SMILES: *CNCCN1CCO[C@H](CN(C)C(*)=O)C1]

In some embodiments, L is

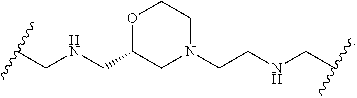 [SMILES: *CNCCN1CCO[C@@H](CNC*)C1]

In some embodiments, L is

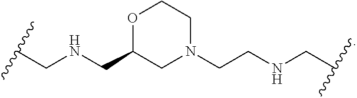 [SMILES: *CNCCN1CCO[C@H](CNC*)C1]

In some embodiments, L is

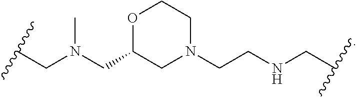 [SMILES: *CNCCN1CCO[C@@H](CN(C)C*)C1]

In some embodiments, L is

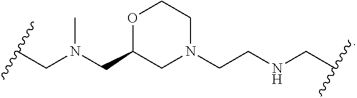 [SMILES: *CNCCN1CCO[C@H](CN(C)C*)C1]

In some embodiments, L is

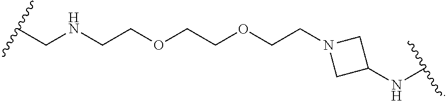 [SMILES: *CNCCOCCOCCN1CC(N*)C1]

In some embodiments, L is

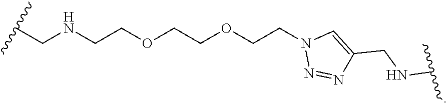 [SMILES: *CNCCOCCOCCn1cc(CN*)nn1]

In some embodiments, L is

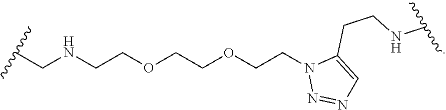 [SMILES: *CNCCOCCOCCn1nncc1CCN*]

In some embodiments, L is

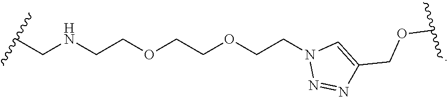 [SMILES: *CNCCOCCOCCn1cc(CO*)nn1]

In some embodiments, L is

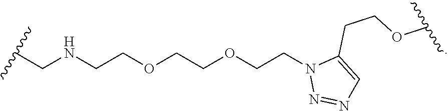 [SMILES: *CNCCOCCOCCn1nncc1CCO*]

In some embodiments, L is

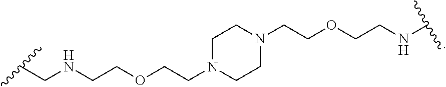 [SMILES: *CNCCOCCN1CCN(CCOCCN*)CC1]

In some embodiments, L is

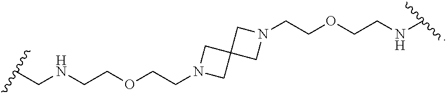 [SMILES: *CNCCOCCN1CC2(CN(CCOCCN*)C2)C1]

In some embodiments, L is

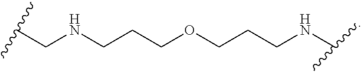 [SMILES: *CNCCCOCCCN*]

In some embodiments, L is

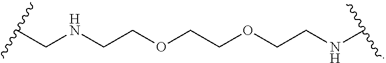 [SMILES: *CNCCOCCOCCN*]

In some embodiments, L is

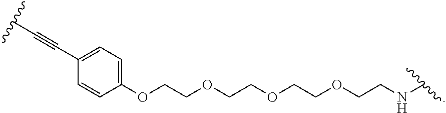 [SMILES: *C#Cc1ccc(OCCOCCOCCOCCN*)cc1]

In some embodiments, L is

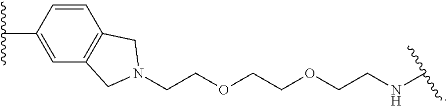 [SMILES: *NCCOCCOCCN1Cc2ccc(*)cc2C1]

In some embodiments, L is

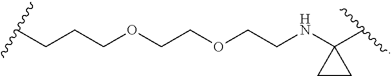 [SMILES: *CCCOCCOCCNC1(*)CC1]

In some embodiments, L is

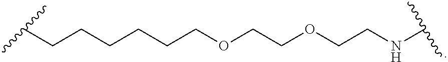 [SMILES: *CCCCCCOCCOCCN*]

In some embodiments, L is

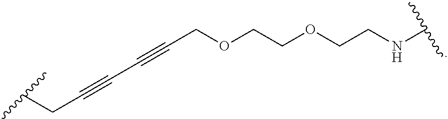 [SMILES: *CC#CC#CCOCCOCCN*]

In some embodiments, L is

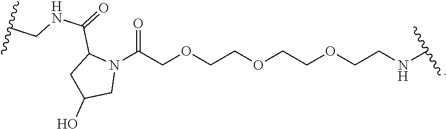 [SMILES: *CNC(=O)C1CC(O)CN1C(=O)COCCOCCOCCN*]

In some embodiments, L is

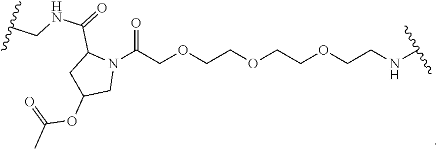 [SMILES: *CNC(=O)C1CC(OC(C)=O)CN1C(=O)COCCOCCOCCN*]

In some embodiments, L is

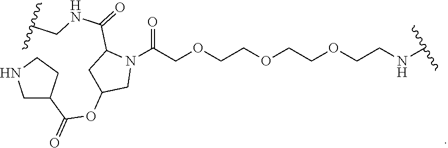 [SMILES: *CNC(=O)C1CC(OC(=O)C2CCNC2)CN1C(=O)COCCOCCOCCN*]

In some embodiments, L is

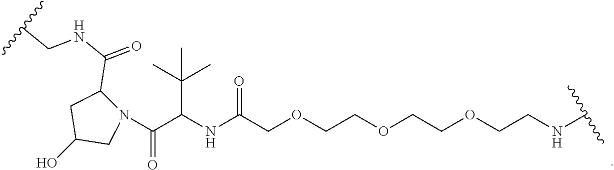 [SMILES: *CNC(=O)C1CC(O)CN1C(=O)C(NC(=O)COCCOCCOCCN*)C(C)(C)C]

In some embodiments, L is

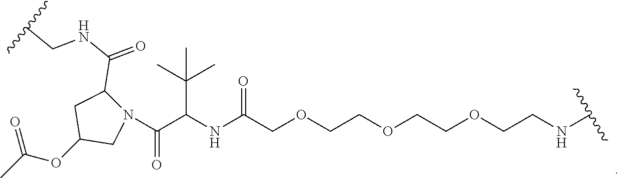 [SMILES: *CNC(=O)C1CC(OC(C)=O)CN1C(=O)C(NC(=O)COCCOCCOCCN*)C(C)(C)C]

In some embodiments, L is

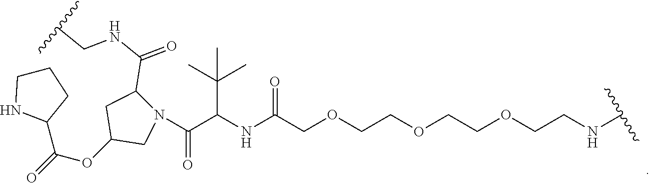 [SMILES: *CNC(=O)C1CC(OC(=O)C2CCCN2)CN1C(=O)C(NC(=O)COCCOCCOCCN*)C(C)(C)C]

In some embodiments, L is

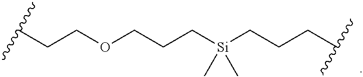 [SMILES: *CCC[Si](C)(C)CCCOCC*]

In some embodiments, L is

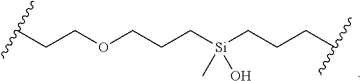 [SMILES: *CCC[Si](C)(O)CCCOCC*]

In some embodiments, L is

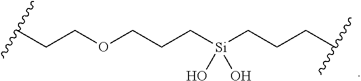 [SMILES: *CCC[Si](O)(O)CCCOCC*]

In some embodiments, L is

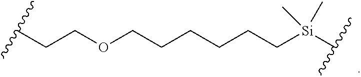 [SMILES: *CCOCCCCCC[Si](*)(C)C]

In some embodiments, L is

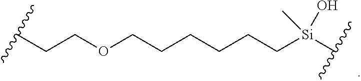 [SMILES: *CCOCCCCCC[Si](*)(C)O]

In some embodiments, L is

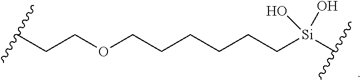 [SMILES: *CCOCCCCCC[Si](*)(O)O]

In some embodiments, L is

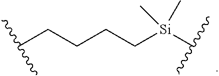 [SMILES: *CCCC[Si](*)(C)C]

In some embodiments, L is

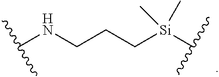 [SMILES: *NCCC[Si](*)(C)C]

In some embodiments, L is

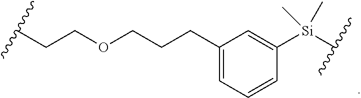 [SMILES: *CCOCCCc1cccc([Si](*)(C)C)c1]

In some embodiments, L is

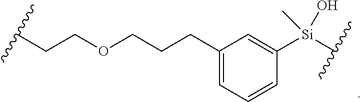 [SMILES: *CCOCCCc1cccc([Si](*)(C)O)c1]

In some embodiments, L is

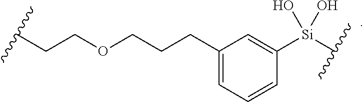 [SMILES: *CCOCCCc1cccc([Si](*)(O)O)c1]

In some embodiments, L is

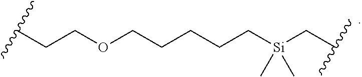 [SMILES: *CCOCCCCC[Si](C)(C)C*]

In some embodiments, L is

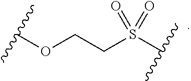 [SMILES: *OCCS(*)(=O)=O]

In some embodiments, L is

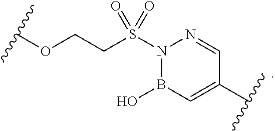 [SMILES: *OCCS(=O)(=O)N1N=CC(*)=CB1O]

In some embodiments, L is

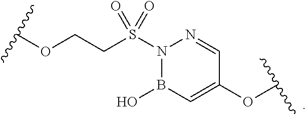 [SMILES: *OCCS(=O)(=O)N1N=CC(O*)=CB1O]

In some embodiments, L is

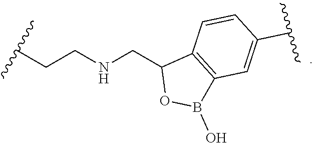 [SMILES: *CCNCC1OB(O)c2cc(*)ccc21]

In some embodiments, L is

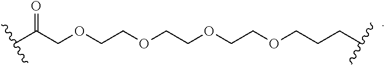 [SMILES: *CCCOCCOCCOCCOCC(*)=O]

In some embodiments, L is

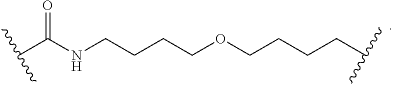 [SMILES: *CCCCOCCCCNC(*)=O]

In some embodiments, L is

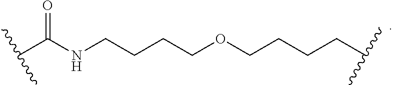 [SMILES: *CCCCOCCCCNC(*)=O]

In some embodiments, L is

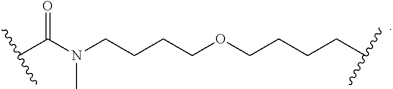 [SMILES: *CCCCOCCCCN(C)C(*)=O]

In some embodiments, L is

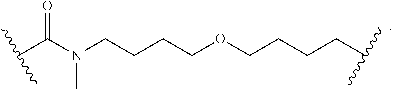 [SMILES: *CCCCOCCCCN(C)C(*)=O]

In some embodiments, L is

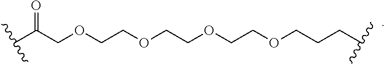 [SMILES: *CCCOCCOCCOCCOCC(*)=O]

In some embodiments, L is

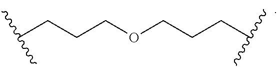 [SMILES: *CCCOCCC*]

In some embodiments, L is

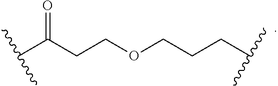 [SMILES: *CCCOCCC(*)=O]

In some embodiments, L is

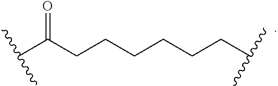 [SMILES: *CCCCCCC(*)=O]

In some embodiments, L is

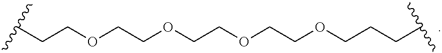 [SMILES: *CCCOCCOCCOCCOCC*]

In some embodiments, L is

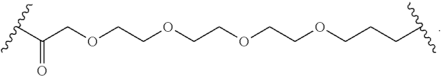 [SMILES: *CCCOCCOCCOCCOCC(*)=O]

In some embodiments, L is

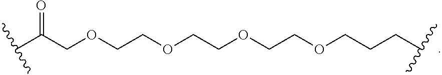 [SMILES: *CCCOCCOCCOCCOCC(*)=O]

In some embodiments, L is

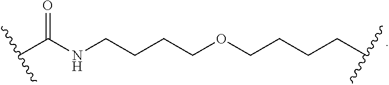 [SMILES: *CCCCOCCCCNC(*)=O]

In some embodiments, L is

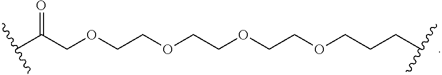 [SMILES: *CCCOCCOCCOCCOCC(*)=O]

In some embodiments, L is

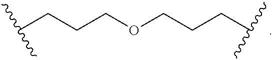 [SMILES: *CCCOCCC*]

In some embodiments, L is

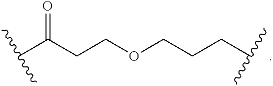 [SMILES: *CCCOCCC(*)=O]

In some embodiments, L is

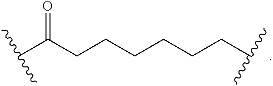 [SMILES: *CCCCCCC(*)=O]

In some embodiments, L is

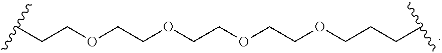 [SMILES: *CCCOCCOCCOCCOCC*]

In some embodiments, L is

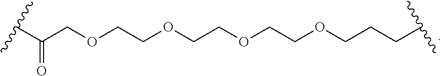 [SMILES: *CCCOCCOCCOCCOCC(*)=O]

In some embodiments, L is

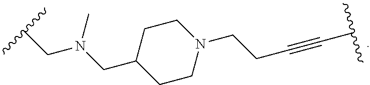 [SMILES: *C#CCCN1CCC(CN(C)C*)CC1]

In some embodiments, L is

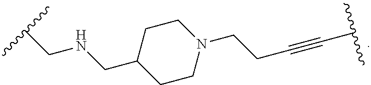 [SMILES: *C#CCCN1CCC(CNC*)CC1]

In some embodiments, L is

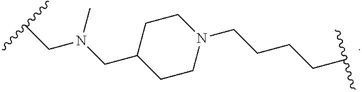 [SMILES: *CCCCN1CCC(CN(C)C*)CC1]

In some embodiments, L is

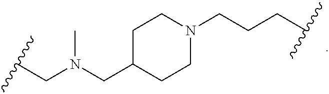 [SMILES: *CCCN1CCC(CN(C)C*)CC1]

In some embodiments, L is

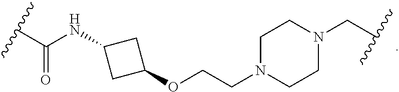 [SMILES: *CN1CCN(CCO[C@H]2C[C@H](NC(*)=O)C2)CC1]

n some embodiments, L is

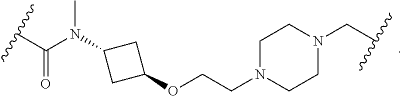 [SMILES: *CN1CCN(CCO[C@H]2C[C@H](N(C)C(*)=O)C2)CC1]

In some embodiments, L is

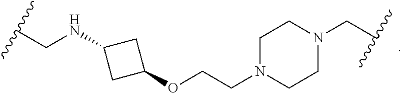 [SMILES: *CN[C@H]1C[C@H](OCCN2CCN(C*)CC2)C1]

In some embodiments, L is

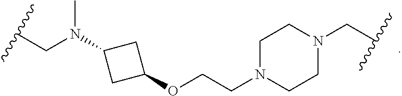 [SMILES: *CN1CCN(CCO[C@H]2C[C@H](N(C)C*)C2)CC1]

In some embodiments, L is

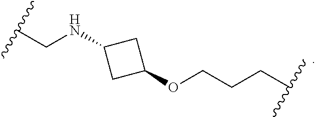 [SMILES: *CCCO[C@H]1C[C@H](NC*)C1]

In some embodiments, L is

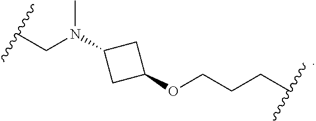 [SMILES: *CCCO[C@H]1C[C@H](N(C)C*)C1]

In some embodiments, L is

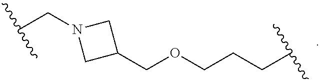 [SMILES: *CCCOCC1CN(C*)C1]

In some embodiments, L is

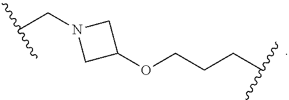 [SMILES: *CCCOC1CN(C*)C1]

In some embodiments, L is

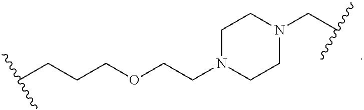 [SMILES: *CCCOCCN1CCN(C*)CC1]

In some embodiments, L is

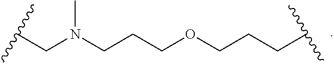 [SMILES: *CCCOCCCN(C)C*]

In some embodiments, L is

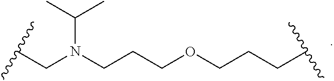 [SMILES: *CCCOCCCN(C*)C(C)C]

In some embodiments, L is

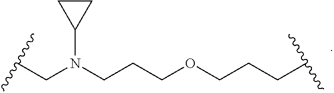 [SMILES: *CCCOCCCN(C*)C1CC1]

In some embodiments, L is

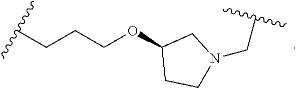 [SMILES: *CCCO[C@@H]1CCN(C*)C1]

In some embodiments, L is

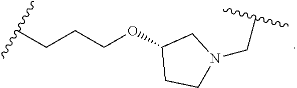 [SMILES: *CCCO[C@H]1CCN(C*)C1]

In some embodiments, L is

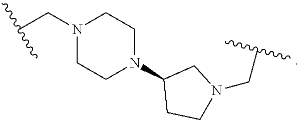 [SMILES: *CN1CCN([C@@H]2CCN(C*)C2)CC1]

In some embodiments, L is

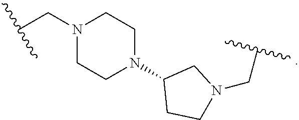 [SMILES: *CN1CCN([C@H]2CCN(C*)C2)CC1]

In some embodiments, L is

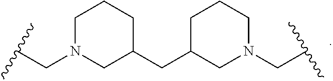 [SMILES: *CN1CCCC(CC2CCCN(C*)C2)C1]

In some embodiments, L is

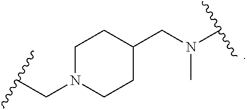 [SMILES: *CN1CCC(CN(*)C)CC1]

In some embodiments, L is

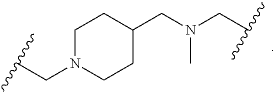 [SMILES: *CN(C)CC1CCN(C*)CC1]

In some embodiments, L is

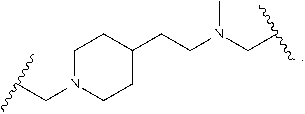 [SMILES: *CN(C)CCC1CCN(C*)CC1]

In some embodiments, L is

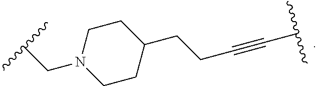 [SMILES: *C#CCCC1CCN(C*)CC1]

In some embodiments, L is

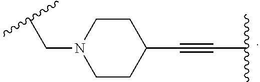 [SMILES: *C#CC1CCN(C*)CC1]

In some embodiments, L is

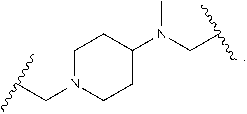 [SMILES: *CN1CCC(N(C)C*)CC1]

In some embodiments, L is

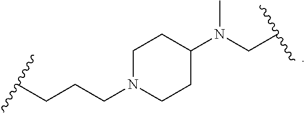 [SMILES: *CCCN1CCC(N(C)C*)CC1]

In some embodiments, L is

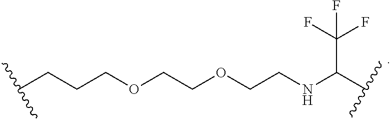 [SMILES: *CCCOCCOCCNC(*)C(F)(F)F]

In some embodiments, L is

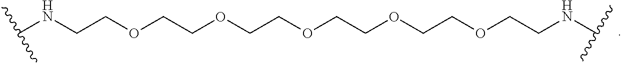 [SMILES: *NCCOCCOCCOCCOCCOCCN*]

In some embodiments, L is

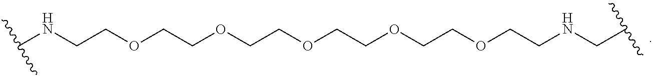 [SMILES: *CNCCOCCOCCOCCOCCOCCN*]

In some embodiments, L is

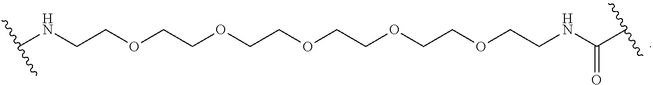 [SMILES: *NCCOCCOCCOCCOCCOCCNC(*)=O]

In some embodiments, L is

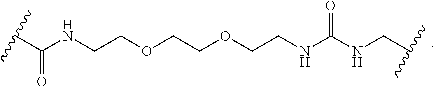 [SMILES: *CNC(=O)NCCOCCOCCNC(*)=O]

In some embodiments, L is

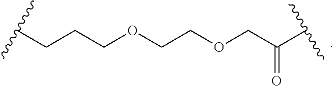 [SMILES: *CCCOCCOCC(*)=O]

In some embodiments, L is

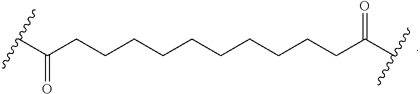 [SMILES: *C(=O)CCCCCCCCCCC(*)=O]

In some embodiments, L is

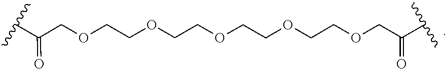 [SMILES: *C(=O)COCCOCCOCCOCCOCC(*)=O]

In some embodiments, L is

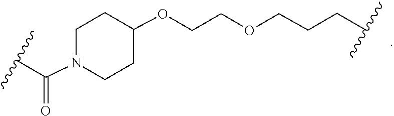 [SMILES: *CCCOCCOC1CCN(C(*)=O)CC1]

In some embodiments, L is

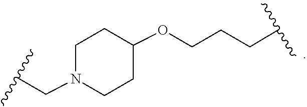 [SMILES: *CCCOC1CCN(C*)CC1]

In some embodiments, L is

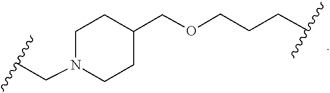 [SMILES: *CCCOCC1CCN(C*)CC1]

In some embodiments, L is

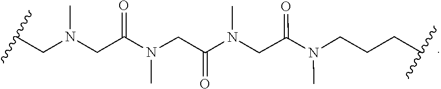 [SMILES: *CCCN(C)C(=O)CN(C)C(=O)CN(C)C(=O)CN(C)C*]

In some embodiments, L is

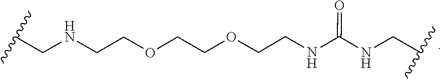 [SMILES: *CNCCOCCOCCNC(=O)NC*]

In some embodiment, L is

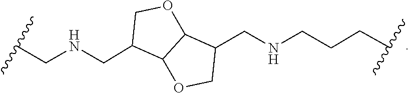 [SMILES: *CCCNCC1COC2C(CNC*)COC12]

In some embodiments, L is

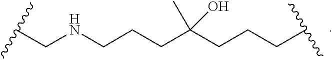 [SMILES: *CCCC(C)(O)CCCNC*]

In some embodiments, L is

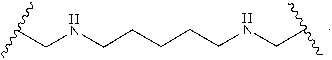 [SMILES: *CNCCCCCNC*]

In some embodiments, L is

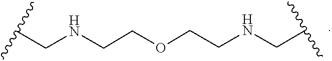 [SMILES: *CNCCOCCNC*]

In some embodiments, L is

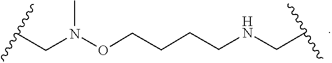 [SMILES: *CNCCCCON(C)C*]

In some embodiments, L is

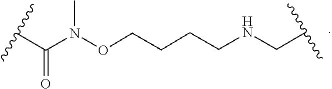 [SMILES: *CNCCCCON(C)C(*)=O]

In some embodiments, L is

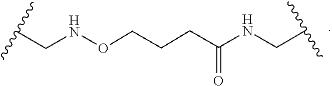 [SMILES: *CNOCCCC(=O)NC*]

In some embodiments, L is

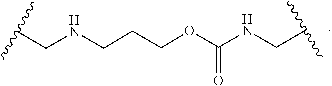 [SMILES: *CNCCCOC(=O)NC*]

In some embodiments, L is

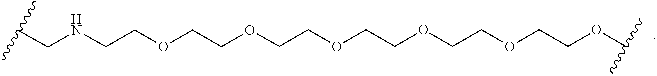 [SMILES: *CNCCOCCOCCOCCOCCOCCO*]

In some embodiments, L is

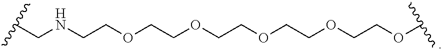 [SMILES: *CNCCOCCOCCOCCOCCO*]

In some embodiments, L is

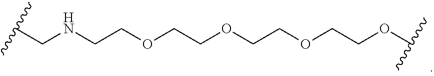 [SMILES: *CNCCOCCOCCOCCO*]

In some embodiments, L is

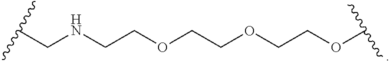 [SMILES: *CNCCOCCOCCO*]

In some embodiments, L is

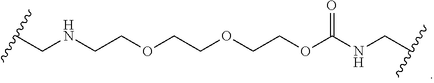 [SMILES: *CNCCOCCOCCOC(=O)NC*]

In some embodiments, L is

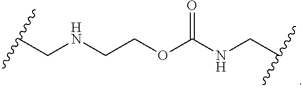 [SMILES: *CNCCOC(=O)NC*]

In some embodiments, L is

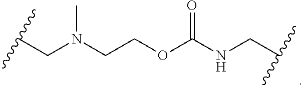 [SMILES: *CNC(=O)OCCN(C)C*]

In some embodiments, L is

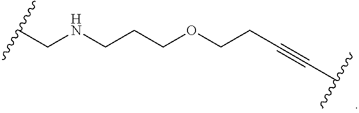 [SMILES: *C#CCCOCCCNC*]

In some embodiments, L is

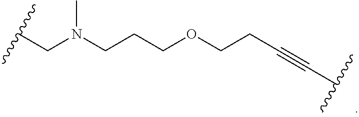 [SMILES: *C#CCCOCCCN(C)C*]

In some embodiments, L is

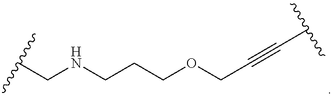 [SMILES: *C#CCOCCCNC*]

In some embodiments, L is

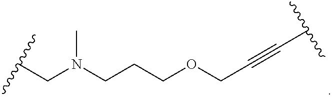 [SMILES: *C#CCOCCCN(C)C*]

In some embodiments, L is

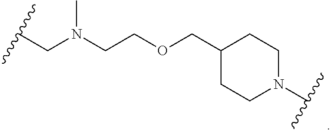 [SMILES: *CN(C)CCOCC1CCN(*)CC1]

In some embodiments, L is

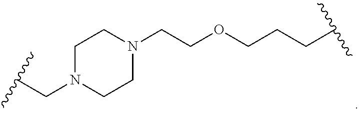 [SMILES: *CCCOCCN1CCN(C*)CC1]

In some embodiments, L is

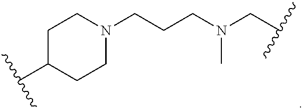 [SMILES: *CN(C)CCCN1CCC(*)CC1]

In some embodiments, L is

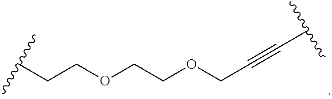 [SMILES: *C#CCOCCOCC*]

In some embodiments, L is

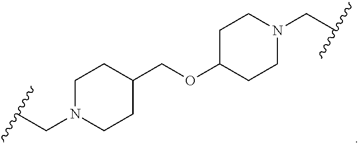 [SMILES: *CN1CCC(COC2CCN(C*)CC2)CC1]

In some embodiments, L is

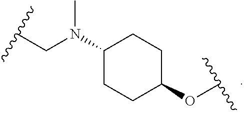 [SMILES: *CN(C)[C@H]1CC[C@H](O*)CC1]

In some embodiments, L is

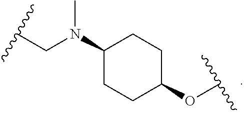 [SMILES: *CN(C)[C@H]1CC[C@@H](O*)CC1]

In some embodiments, L is

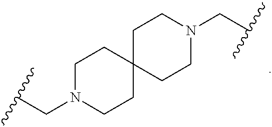 [SMILES: *CN1CCC2(CC1)CCN(C*)CC2]

In some embodiments, L is

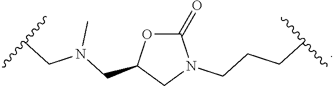 [SMILES: *CCCN1C[C@@H](CN(C)C*)OC1=O]

In some embodiments, L is

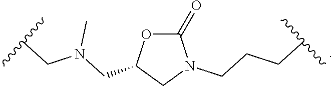 [SMILES: *CCCN1C[C@H](CN(C)C*)OC1=O]

In some embodiments, L is

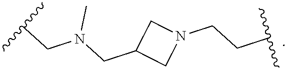 [SMILES: *CCN1CC(CN(C)C*)C1]

In some embodiments, L is

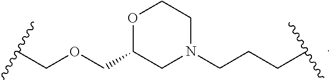 [SMILES: *CCCN1CCO[C@@H](COC*)C1]

In some embodiments, L is

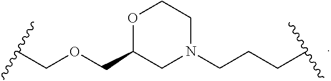 [SMILES: *CCCN1CCO[C@H](COC*)C1]

In some embodiments, L is

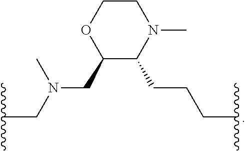 [SMILES: *CCC[C@@H]1[C@@H](CN(C)C*)OCCN1C]

In some embodiments, L is

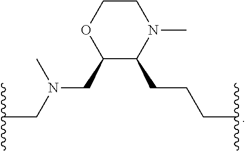 [SMILES: *CCC[C@H]1[C@@H](CN(C)C*)OCCN1C]

In some embodiments, L is

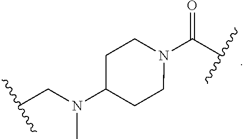 [SMILES: *CN(C)C1CCN(C(*)=O)CC1]

In some embodiments, L is

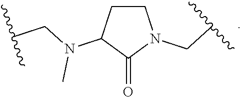 [SMILES: *CN1CCC(N(C)C*)C1=O]

In some embodiments, L is

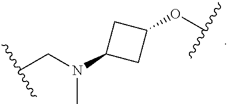 [SMILES: *CN(C)[C@H]1C[C@H](O*)C1]

In some embodiments, L is

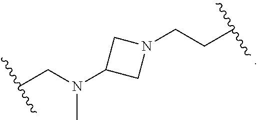 [SMILES: *CCN1CC(N(C)C*)C1]

In some embodiments, L is

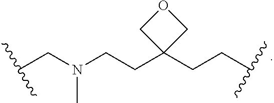 [SMILES: *CCC1(CCN(C)C*)COC1]

In some embodiments, L is

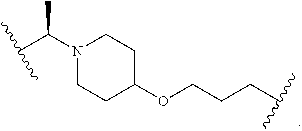 [SMILES: *CCCOC1CCN([C@@H](*)C)CC1]

In some embodiments, L is

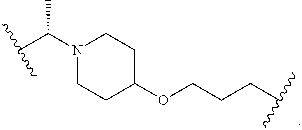 [SMILES: *CCCOC1CCN([C@H](*)C)CC1]

In some embodiments, L is

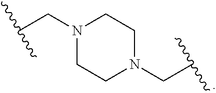 [SMILES: *CN1CCN(C*)CC1]

In some embodiments, L is

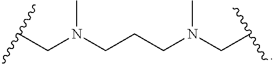 [SMILES: *CN(C)CCCN(C)C*]

In some embodiments, L is

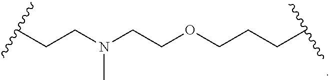 [SMILES: *CCCOCCN(C)CC*]

In some embodiments, L is

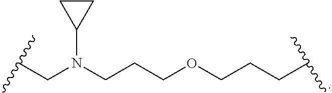 [SMILES: *CCCOCCCN(C*)C1CC1]

In some embodiments, L is

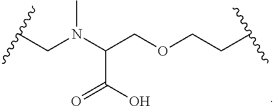 [SMILES: *CCOCC(C(=O)O)N(C)C*]

In some embodiments, L is

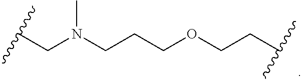 [SMILES: *CCOCCCN(C)C*]

In some embodiments, L is

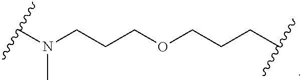 [SMILES: *CCCOCCCN(*)C]

In some embodiments, L is

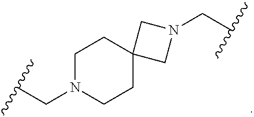 [SMILES: *CN1CCC2(CC1)CN(C*)C2]

In some embodiments, L is

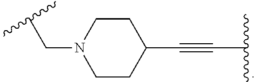 [SMILES: *C#CC1CCN(C*)CC1]

In some embodiments, L is

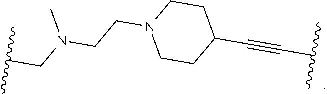 [SMILES: *C#CC1CCN(CCN(C)C*)CC1]

In some embodiments, L is

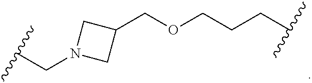 [SMILES: *CCCOCC1CN(C*)C1]

In some embodiments, L is a covalent bond. In some embodiments, L is

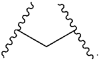 [SMILES: *C*]

In some embodiments, L is

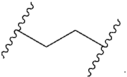 [SMILES: *CC*]

In some embodiments, L is

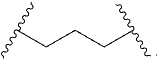 [SMILES: *CCC*]

In some embodiments, L is

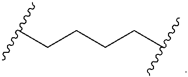 [SMILES: *CCCC*]

In some embodiments, L is

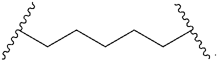 [SMILES: *CCCCC*]

In some embodiments, L is

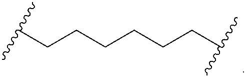 [SMILES: *CCCCCC*]

In some embodiments, L is

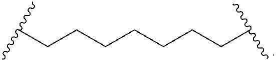 [SMILES: *CCCCCCC*]

In some embodiments, L is

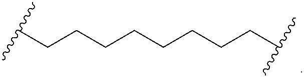 [SMILES: *CCCCCCCC*]

In some embodiments, L is

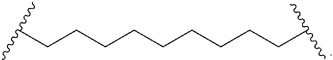 [SMILES: *CCCCCCCCC*]

In some embodiments, L is a covalent bond. In some embodiments, L is

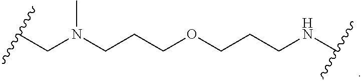 [SMILES: *CN(C)CCCOCCCN*]

In some embodiments, L is

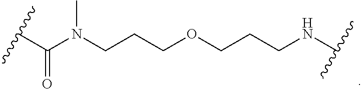 [SMILES: *NCCCOCCCN(C)C(*)=O]

In some embodiments, L is

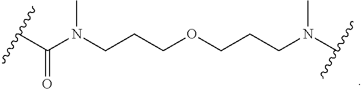 [SMILES: *C(=O)N(C)CCCOCCCN(*)C]

In some embodiments, L is

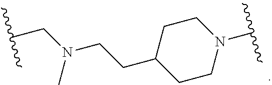 [SMILES: *CN(C)CCC1CCN(*)CC1]

In some embodiments, L is

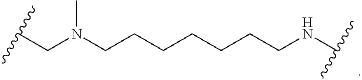 [SMILES: *CN(C)CCCCCCCN*]

In some embodiments, L is

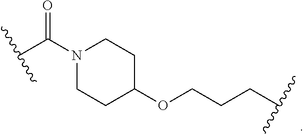 [SMILES: *CCCOC1CCN(C(*)=O)CC1]

In some embodiments, L is

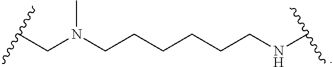 [SMILES: *CN(C)CCCCCCN*]

In some embodiments, L is

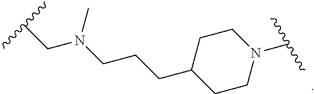 [SMILES: *CN(C)CCCC1CCN(*)CC1]

In some embodiments, L is

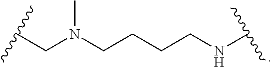 [SMILES: *CN(C)CCCCN*]

In some embodiments, L is

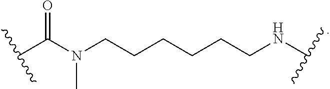 [SMILES: *NCCCCCCN(C)C(*)=O]

In some embodiments, L is

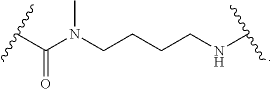 [SMILES: *NCCCCN(C)C(*)=O]

In some embodiments, L is

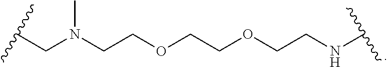 [SMILES: *CN(C)CCOCCOCCN*]

In some embodiments, L is

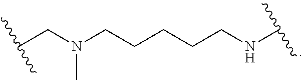 [SMILES: *CN(C)CCCCCN*]

In some embodiments, L is

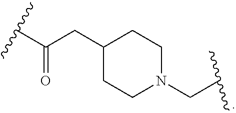 [SMILES: *CN1CCC(CC(*)=O)CC1]

In some embodiments, L is

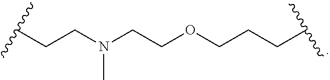 [SMILES: *CCCOCCN(C)CC*]

In some embodiments, L is

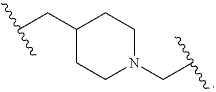 [SMILES: *CC1CCN(C*)CC1]

In some embodiments, L is

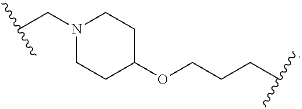 [SMILES: *CCCOC1CCN(C*)CC1]

In some embodiments, L is

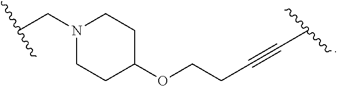 [SMILES: *C#CCCOC1CCN(C*)CC1]

In some embodiments, L is

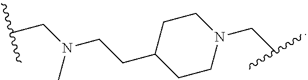 [SMILES: *CN(C)CCC1CCN(C*)CC1]

In some embodiments, L is

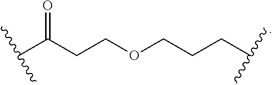 [SMILES: *CCCOCCC(*)=O]

In some embodiments, L is

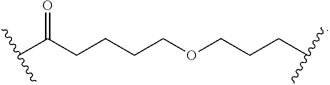 [SMILES: *CCCOCCCCC(*)=O]

In some embodiments, L is

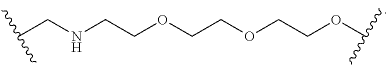 [SMILES: *CNCCOCCOCCO*]

In some embodiments, L is

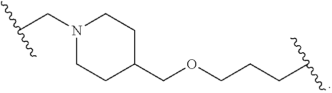 [SMILES: *CCCOCC1CCN(C*)CC1]

In some embodiments, L is

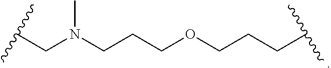 [SMILES: *CCCOCCCN(C)C* ; "."]

In some embodiments, L is

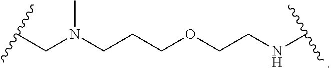 [SMILES: *CN(C)CCCOCCN* ; "."]

In some embodiments, L is

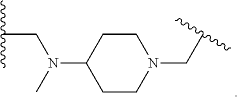 [SMILES: *CN1CCC(N(C)C*)CC1 ; "."]

In some embodiments, L is

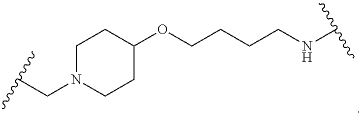 [SMILES: *CN1CCC(OCCCCN*)CC1 ; "."]

In some embodiments, L is

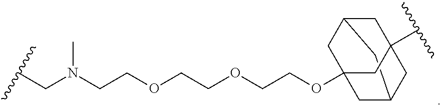 [SMILES: *CN(C)CCOCCOCCOC12CC3CC(CC(*)(C3)C1)C2 ; "."]

In some embodiments, L is

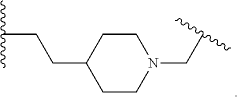 [SMILES: *CCC1CCN(C*)CC1 ; "."]

In some embodiments, L is

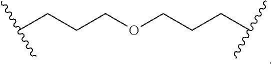 [SMILES: *CCCOCCC* ; "."]

In some embodiments, L is

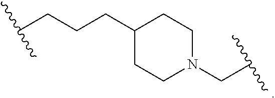 [SMILES: *CCCC1CCN(C*)CC1 ; "."]

In some embodiments, L is

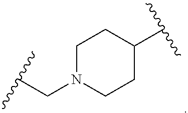 [SMILES: *CN1CCC(*)CC1 ; "."]

In some embodiments, L is

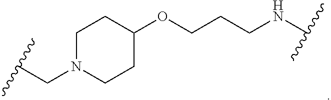 [SMILES: *CN1CCC(OCCCN*)CC1 ; "."]

In some embodiments, L is

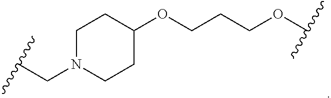 [SMILES: *CN1CCC(OCCCO*)CC1 ; "."]

In some embodiments, L is

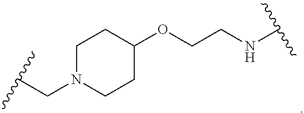 [SMILES: *CN1CCC(OCCN*)CC1 ; "."]

In some embodiments, L is

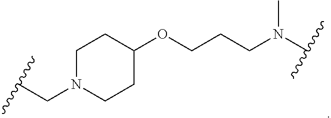 [SMILES: *CN1CCC(OCCCN(*)C)CC1 ; "."]

In some embodiments, L is

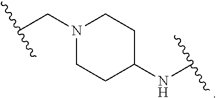 [SMILES: *CN1CCC(N*)CC1 ; "."]

In some embodiments, L is

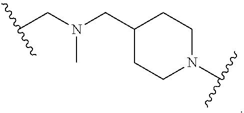 [SMILES: *CN(C)CC1CCN(*)CC1 ; "."]

In some embodiments, L is

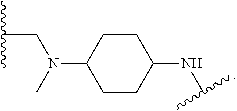 [SMILES: *CN(C)C1CCC(N*)CC1]

In some embodiments, L is

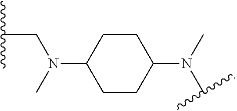 [SMILES: *CN(C)C1CCC(N(*)C)CC1]

In some embodiments, L is

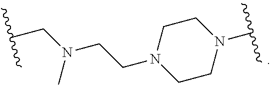 [SMILES: *CN(C)CCN1CCN(*)CC1]

In some embodiments, L is

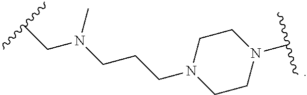 [SMILES: *CN(C)CCCN1CCN(*)CC1]

In some embodiments, L is

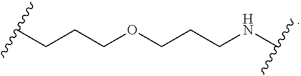 [SMILES: *CCCOCCCN*]

In some embodiments, L is

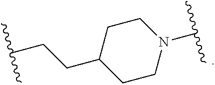 [SMILES: *CCC1CCN(*)CC1]

In some embodiments, L is

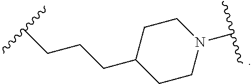 [SMILES: *CCCC1CCN(*)CC1]

In some embodiments, L is

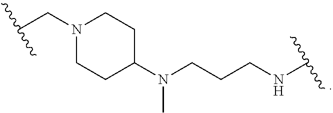 [SMILES: *CN1CCC(N(C)CCCN*)CC1]

In some embodiments, L is

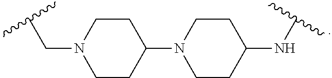 [SMILES: *CN1CCC(N2CCC(N*)CC2)CC1]

In some embodiments, L is

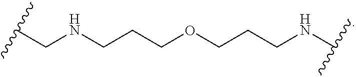 [SMILES: *CNCCCOCCCN*]

In some embodiments, L is

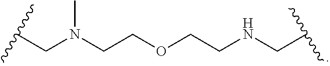 [SMILES: *CNCCOCCN(C)C*]

In some embodiments, L is

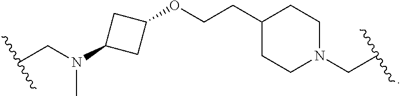 [SMILES: *CN1CCC(CCO[C@H]2C[C@H](N(C)C*)C2)CC1]

In some embodiments, L is

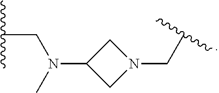 [SMILES: *CN1CC(N(C)C*)C1]

In some embodiments, L is

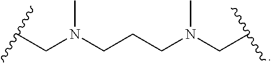 [SMILES: *CN(C)CCCN(C)C*]

In some embodiments, L is

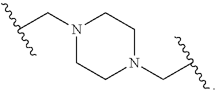 [SMILES: *CN1CCN(C*)CC1]

In some embodiments, L is

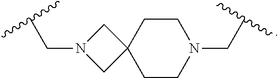 [SMILES: *CN1CCC2(CC1)CN(C*)C2]

In some embodiments, L is

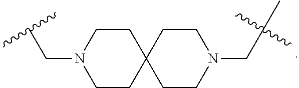 [SMILES: *CN1CCC2(CC1)CCN(C*)CC2]

In some embodiments, L is

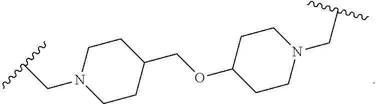 [SMILES: *CN1CCC(COC2CCN(C*)CC2)CC1]

In some embodiments, L is

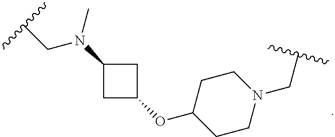 [SMILES: *CN1CCC(O[C@H]2C[C@H](N(C)C*)C2)CC1]

In some embodiments, L is

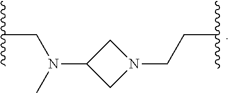 [SMILES: *CCN1CC(N(C)C*)C1]

In some embodiments, L is

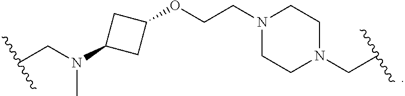 [SMILES: *CN1CCN(CCO[C@H]2C[C@H](N(C)C*)C2)CC1]

In some embodiments, L is

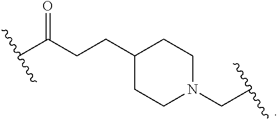 [SMILES: *CN1CCC(CCC(*)=O)CC1]

In some embodiments, L is

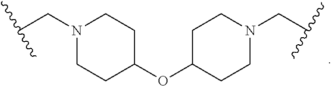 [SMILES: *CN1CCC(OC2CCN(C*)CC2)CC1]

In some embodiments, L is

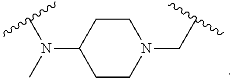 [SMILES: *CN1CCC(N(*)C)CC1]

In some embodiments, L is

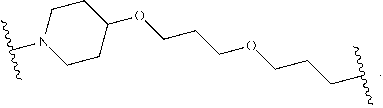 [SMILES: *CCCOCCCOC1CCN(*)CC1]

In In some embodiments, L is

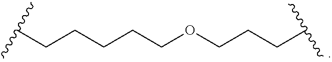 [SMILES: *CCCCCOCCC*]

In some embodiments, L is

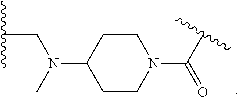 [SMILES: *CN(C)C1CCN(C(*)=O)CC1]

In some embodiments, L is

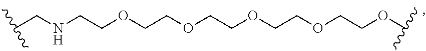 [SMILES: *CNCCOCCOCCOCCOCCO*]

In some embodiments, L is

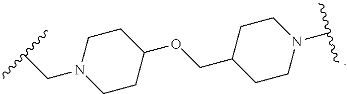 [SMILES: *CN1CCC(OCC2CCN(*)CC2)CC1]

In some embodiments, L is

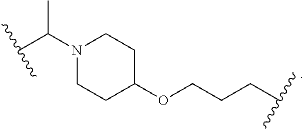 [SMILES: *CCCOC1CCN(C(*)C)CC1]

In some embodiments, L is

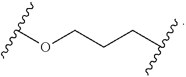 [SMILES: *CCCO*]

In some embodiments, L is

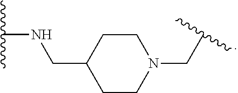 [SMILES: *CN1CCC(CN*)CC1]

In some embodiments, L is

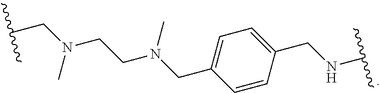 [SMILES: *CN(C)CCN(C)Cc1ccc(CN*)cc1]

In some embodiments, L is

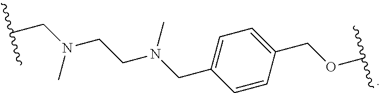 [SMILES: *CN(C)CCN(C)Cc1ccc(CO*)cc1]

In some embodiments, L is

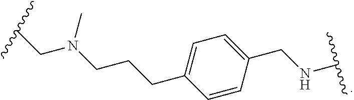 [SMILES: *CN(C)CCCc1ccc(CN*)cc1]

In some embodiments, L is

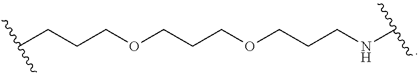 [SMILES: *CCCOCCCOCCCN*]

In some embodiments, L is

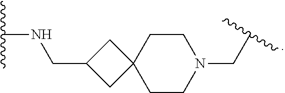 [SMILES: *CN1CCC2(CC1)CC(CN*)C2]

In some embodiments, L is

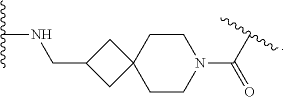 [SMILES: *NCC1CC2(CCN(C(*)=O)CC2)C1]

In some embodiments, L is

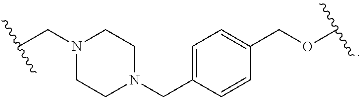 [SMILES: *CN1CCN(Cc2ccc(CO*)cc2)CC1]

In some embodiments, L is

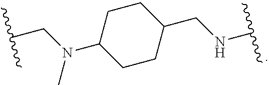 [SMILES: *CN(C)C1CCC(CN*)CC1]

In some embodiments, L is

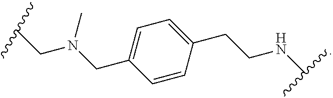 [SMILES: *CN(C)Cc1ccc(CCN*)cc1]

In some embodiments, L is

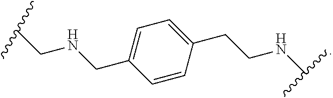 [SMILES: *CNCc1ccc(CCN*)cc1]

In some embodiments, L is

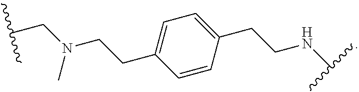 [SMILES: *CN(C)CCc1ccc(CCN*)cc1]

In some embodiments, L is

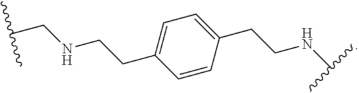 [SMILES: *CNCCc1ccc(CCN*)cc1]

In some embodiments, L is

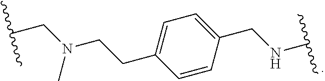 [SMILES: *CN(C)CCc1ccc(CN*)cc1]

In some embodiments, L is

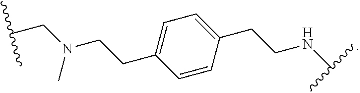 [SMILES: *CN(C)CCc1ccc(CCN*)cc1]

In some embodiments, L is

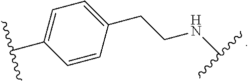 [SMILES: *NCCc1ccc(*)cc1]

In In some embodiments, L is In some

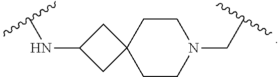 [SMILES: *CN1CCC2(CC1)CC(N*)C2]

In In some embodiments, L is

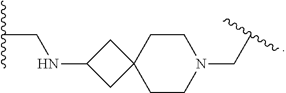 [SMILES: *CNC1CC2(CCN(C*)CC2)C1]

embodiments, L is

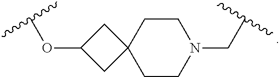 [SMILES: *CN1CCC2(CC1)CC(O*)C2]

In some embodiments, L is

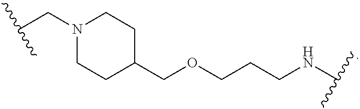 [SMILES: *CN1CCC(COCCCN*)CC1]

In some embodiments, L is

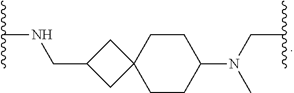 [SMILES: *CN(C)C1CCC2(CC1)CC(CN*)C2]

In some embodiments, L is

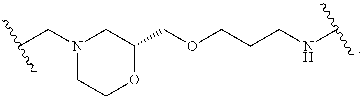 [SMILES: *CN1CCO[C@@H](COCCCN*)C1]

In some embodiments, L is

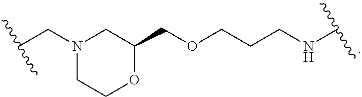 [SMILES: *CN1CCO[C@H](COCCCN*)C1]

In some embodiments, L is

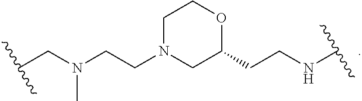 [SMILES: *CN(C)CCN1CCO[C@H](CCN*)C1]

In some embodiments, L is

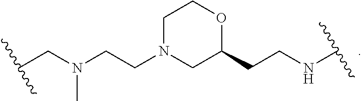 [SMILES: *CN(C)CCN1CCO[C@@H](CCN*)C1]

In some embodiments, L is

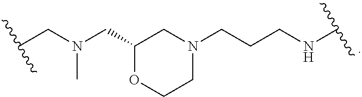 [SMILES: *CN(C)C[C@@H]1CN(CCCN*)CCO1]

In some embodiments, L is

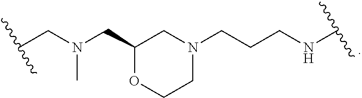 [SMILES: *CN(C)C[C@H]1CN(CCCN*)CCO1]

In some embodiments, L is

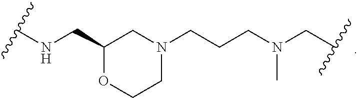 [SMILES: *CN(C)CCCN1CCO[C@@H](CN*)C1]

In some embodiments, L is

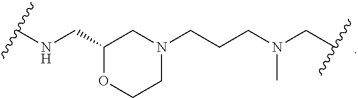 [SMILES: *CN(C)CCCN1CCO[C@H](CN*)C1]

In some embodiments, L is

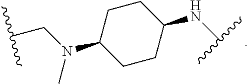 [SMILES: *CN(C)[C@H]1CC[C@@H](N*)CC1]

In some embodiments, L is

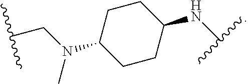 [SMILES: *CN(C)[C@H]1CC[C@H](N*)CC1]

In some embodiments, L is

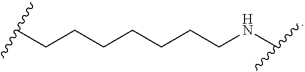 [SMILES: *CCCCCCCN*]

In some embodiments, L is

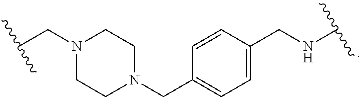 [SMILES: *CN1CCN(Cc2ccc(CN*)cc2)CC1]

some embodiments, L is

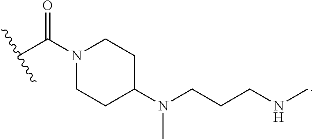 [SMILES: *C(=O)N1CCC(N(C)CCCNC)CC1]

In some embodiments, L is

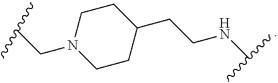 [SMILES: *CN1CCC(CCN*)CC1]

In some embodiments, L is

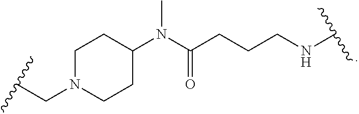 [SMILES: *CN1CCC(N(C)C(=O)CCCN*)CC1]

In some embodiments, L is

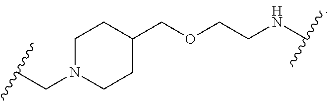 [SMILES: *CN1CCC(COCCN*)CC1]

In some embodiments, L is

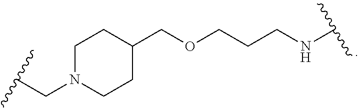 [SMILES: *CN1CCC(COCCCN*)CC1]

In some embodiments, L is

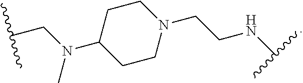 [SMILES: *CN(C)C1CCN(CCN*)CC1]

In some embodiments, L is

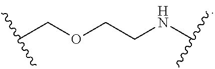 [SMILES: *COCCN*]

In some embodiments, L is

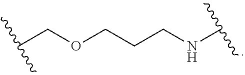 [SMILES: *COCCCN*]

In some embodiments, L is

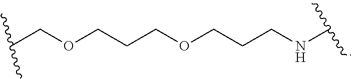 [SMILES: *COCCCOCCCN*]

In some embodiments, L is

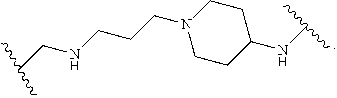 [SMILES: *CNCCCN1CCC(N*)CC1]

In some embodiments, L is selected from those depicted in Table 1, below.

As defined above and described herein, LBM is a ligase binding moiety.

In some embodiments, LBM is an E3 ubiquitin ligase (cereblon) binding moiety

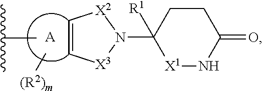

a DCAF 15 E3 ubiquitin ligase binding moiety

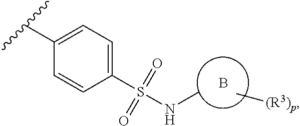

or a VHL E3 ubiquitin ligase binding moiety

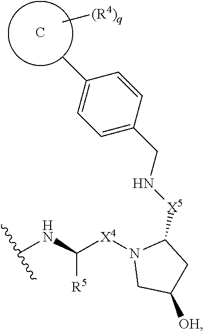

wherein each of X¹, X², and X³ is independently a bivalent moiety selected from a covalent bond, —CH₂—, —C(O)—, —C(S)—, or

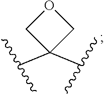 [SMILES: *C1(*)COC1]

each of X⁴ and X⁵ is independently a bivalent moiety selected from —CH₂—, —C(O)—, —C(S)—, or

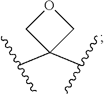 [SMILES: *C1(*)COC1]

R¹ is hydrogen, deuterium, halogen, —CN, —OR, —SR, —S(O)R, —S(O)₂R, —NR₂, or an optionally substituted C₁₋₄ aliphatic;
each of R², R³, and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen or C₁₋₆ aliphatic;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur;
Ring B is selected from 6-membered aryl containing 0-2 nitrogen atoms or a 8-10 membered bicyclic heteroaryl having 1-5 heteroatoms independently selected from nitrogen, oxygen, or sulfur;
Ring C is a selected from 6-membered aryl containing 0-2 nitrogen atoms or a 5-membered heteroaryl with 1-3 heteroatoms independently selected from nitrogen, oxygen or sulfur;
m is 0, 1, 2, 3 or 4;
each of n is independently 1, 2, 3, 4, 5, 6, 7, 8, 9, or 10;
p is 0, 1, 2, 3 or 4;
q is 0, 1, 2, 3 or 4; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, LBM is

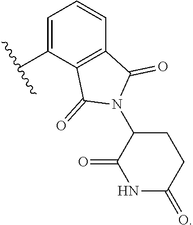 [SMILES: *c1cccc2c1C(=O)N(C1CCC(=O)NC1=O)C2=O]

In some embodiments, LBM is

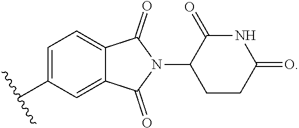 [SMILES: *c1ccc2c(c1)C(=O)N(C1CCC(=O)NC1=O)C2=O]

In some embodiments, LBM is

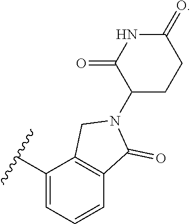 [SMILES: *c1cccc2c1CN(C1CCC(=O)NC1=O)C2=O]

In some embodiments, LBM is

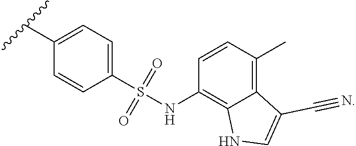 [SMILES: *c1ccc(S(=O)(=O)Nc2ccc(C)c3c(C#N)c[nH]c23)cc1]

In some embodiments, LBM is

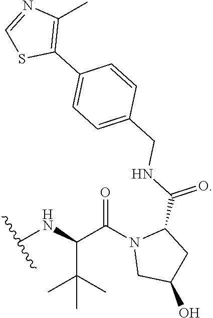 [SMILES: *N[C@@H](C(=O)N1C[C@H](O)C[C@H]1C(=O)NCc1ccc(-c2scnc2C)cc1)C(C)(C)C]

In some embodiments, LBM is

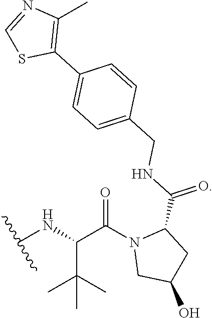 [SMILES: *N[C@H](C(=O)N1C[C@H](O)C[C@H]1C(=O)NCc1ccc(-c2scnc2C)cc1)C(C)(C)C]

In some embodiments, LBM is

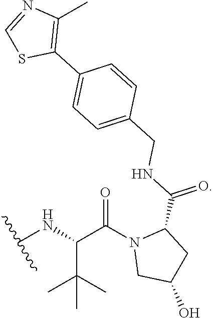 [SMILES: *N[C@H](C(=O)N1C[C@@H](O)C[C@H]1C(=O)NCc1ccc(-c2scnc2C)cc1)C(C)(C)C]

In some embodiments, LBM is a E3 Ubiquitin ligase (cereblon) binding moiety recited in Varfolomeev, E. et al., IAP Antagonists Induce Autoubiquitination of c-IAPs, NF-κB activation, and INFα-Dependent Apoptosis, Cell, 2007, 131(4): 669-81, such as, for example:

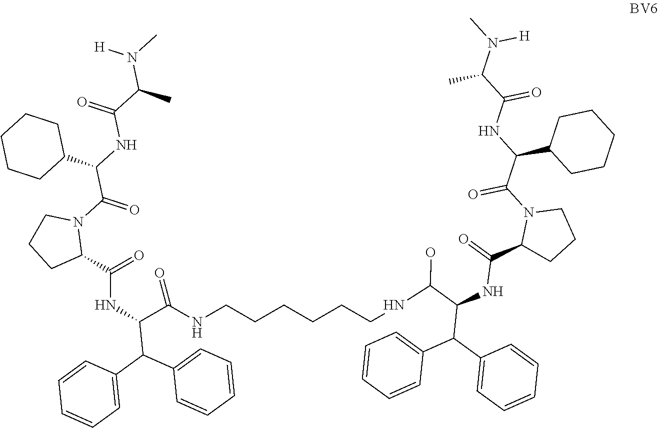 [SMILES: CN[C@@H](C)C(=O)N[C@H](C(=O)N1CCC[C@H]1C(=O)N[C@H](C(O)NCCCCCCNC(=O)[C@@H](NC(=O)[C@@H]1CCCN1C(=O)[C@@H](NC(=O)[C@H](C)NC)C1CCCCC1)C(c1ccccc1)c1ccccc1)C(c1ccccc1)c1ccccc1)C1CCCCC1]

wherein

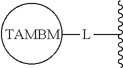

is attached to a modifiable carbon, oxygen, nitrogen or sulfur atom.

In some embodiments, LBM is selected from those depicted in Table 1, below.

As defined above and described herein, each of X¹, X², and X³ is independently a bivalent moiety selected from a covalent bond, —CH₂—, —C(O)—, —C(S)—, or

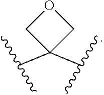 [SMILES: *C1(*)COC1]

In some embodiments, X¹ is a covalent bond, —CH₂—, —C(O)—, —C(S)—, or

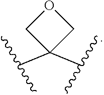 [SMILES: *C1(*)COC1]

In some embodiments, X¹ is selected from those depicted in Table 1, below.

In some embodiments, X² is a covalent bond, —CH₂—, —C(O)—, —C(S)—, or

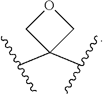 [SMILES: *C1(*)COC1]

In some embodiments, X² is selected from those depicted in Table 1, below.

In some embodiments, X³ is a covalent bond, —CH₂—, —C(O)—, —C(S)—, or

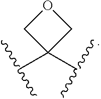 [SMILES: *C1(*)COC1]

In some embodiments, X³ is selected from those depicted in Table 1, below.

As defined above and described herein, each of X⁴ and X⁵ is independently a bivalent moiety selected from —CH₂—, —C(O)—, —C(S)—, or

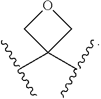 [SMILES: *C1(*)COC1]

In some embodiments, X⁴ is —CH₂—, —C(O)—, —C(S)—, or

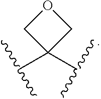 [SMILES: *C1(*)COC1]

In some embodiments, X⁴ is selected from those depicted in Table 1, below.

In some embodiments, X⁵ is —CH₂—, —C(O)—, —C(S)—, or

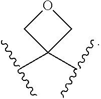 [SMILES: *C1(*)COC1]

In some embodiments, X⁵ is selected from those depicted in Table 1, below.

As defined above and described herein, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is hydrogen, deuterium, halogen, —CN, —OR, —SR, —S(O)R, —S(O)₂R, —NR₂, or an optionally substituted C₁₋₄ aliphatic.

In some embodiments, R¹ is selected from those depicted in Table 1, below.

As defined above and described herein, each of R², R³, and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R² is selected from those depicted in Table 1, below.

In some embodiments, R³ is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R³ is methyl.

In some embodiments, R³ is selected from those depicted in Table 1, below.

In some embodiments, R⁴ is hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R.

In some embodiments, R⁴ is methyl.

In some embodiments, R⁴ is selected from those depicted in Table 1, below.

As defined above and described herein, R⁵ is hydrogen or C₁₋₆ aliphatic.

In some embodiments, R⁵ is t-butyl.

In some embodiments, R⁵ is selected from those depicted in Table 1, below.

As defined above and described herein, each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R⁶ is an optionally substituted C₁₋₆ aliphatic group. In some embodiments, R⁶ is an optionally substituted phenyl. In some embodiments, R⁶ is an optionally substituted 4-7 membered saturated or partially unsaturated heterocyclic ring having 1-2 heteroatoms independently selected from nitrogen, oxygen, and sulfur. In some embodiments, R⁶ is an optionally substituted 5-6 membered heteroaryl ring having 1-4 heteroatoms independently selected from nitrogen, oxygen, and sulfur.

In some embodiments, R⁶ is selected from those depicted in Table 1, below.

As defined above and described herein, Ring A is a fused ring selected from 6-membered aryl containing 0-2 nitrogen atoms, 5 to 7-membered partially saturated carbocyclyl, 5 to 7-membered partially saturated heterocyclyl with 1-2 heteroatoms independently selected from nitrogen, oxygen or sulfur, or 5-membered heteroaryl with 1-3 heteroatoms independently selected from nitrogen, oxygen or sulfur.

In some embodiments Ring A is a fused 6-membered aryl containing 0-2 nitrogen atoms. In some embodiments Ring A is a fused 5 to 7-membered partially saturated carbocyclyl. In some embodiments Ring A is a fused 5 to 7-membered partially saturated heterocyclyl with 1-2 heteroatoms independently selected from nitrogen, oxygen or sulfur. In some embodiments Ring A is a fused 5-membered heteroaryl with 1-3 heteroatoms independently selected from nitrogen, oxygen or sulfur.

In some embodiments, Ring A is a fused phenyl.

In some embodiments, Ring A is selected from those depicted in Table 1, below.

As defined above and described herein, Ring B is selected from 6-membered aryl containing 0-2 nitrogen atoms or a 8-10 membered bicyclic heteroaryl having 1-5 heteroatoms independently selected from nitrogen, oxygen, or sulfur.

In some embodiments, Ring B is a 6-membered aryl containing 0-2 nitrogen atoms. In some embodiments, Ring B is a 8-10 membered bicyclic heteroaryl having 1-5 heteroatoms independently selected from nitrogen, oxygen, or sulfur.

In some embodiments, Ring B is

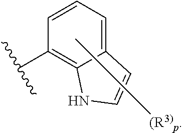

In some embodiments, Ring B is selected from those depicted in Table 1, below.

As defined above and described herein, Ring C is selected from 6-membered aryl containing 0-2 nitrogen atoms or a 5-membered heteroaryl with 1-3 heteroatoms independently selected from nitrogen, oxygen or sulfur.

In some embodiments, Ring C is a 6-membered aryl containing 0-2 nitrogen atoms. In some embodiments, Ring C is a 5-membered heteroaryl with 1-3 heteroatoms independently selected from nitrogen, oxygen or sulfur.

In some embodiments, Ring C is

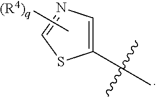

In some embodiments, Ring C is selected from those depicted in Table 1, below.

As defined above and described herein, m is 0, 1, 2, 3 or 4.

In some embodiments, m is 0. In some embodiments, m is 1. In some embodiments, m is 2. In some embodiments, m is 3. In some embodiments, m is 4.

In some embodiments, m is selected from those depicted in Table 1, below.

As defined above and described herein, each of n is independently 1, 2, 3, 4, 5, 6, 7, 8, 9, or 10.

In some embodiments, n is 0. In some embodiments, n is 1. In some embodiments, n is 2. In some embodiments, n is 3. In some embodiments, n is 4. In some embodiments, n is 5. In some embodiments, n is 6. In some embodiments, n is 7. In some embodiments, n is 8. In some embodiments, n is 9. In some embodiments, n is 10.

In some embodiments, n is selected from those depicted in Table 1, below.

As defined above and described herein, p is 0, 1, 2, 3 or 4.

In some embodiments, p is 0. In some embodiments, p is 1. In some embodiments, p is 2. In some embodiments, p is 3. In some embodiments, p is 4.

In some embodiments, p is selected from those depicted in Table 1, below.

As defined above and described herein, q is 0, 1, 2, 3 or 4.

In some embodiments, q is 0. In some embodiments, q is 1. In some embodiments, q is 2. In some embodiments, q is 3. In some embodiments, q is 4.

In some embodiments, q is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is phenyl. In some embodiments, R is a 4-7 membered saturated or partially unsaturated heterocyclic having 1-2 heteroatoms independently selected from nitrogen, oxygen, and sulfur. In some embodiments, R is a 5-6 membered heteroaryl ring having 1-4 heteroatoms independently selected from nitrogen, oxygen, and sulfur. In some embodiments, two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

In certain embodiments, the present invention provides a compound of Formula I as a compound of Formula II-a:

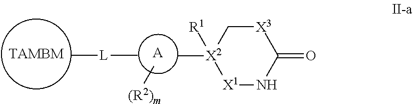

or a pharmaceutically acceptable salt thereof, wherein L and TAMBM are as defined above and described in embodiments herein, wherein:
   X¹ is a bivalent moiety selected from a covalent bond, —CH₂—, —C(O)—, —C(S)—, or

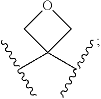 [SMILES: *C1(*)COC1]

X² is a carbon atom or silicon atom;
X³ is a bivalent moiety selected from —CH₂— or —Si(R₂)—;
R¹ is hydrogen, deuterium, halogen, —CN, —OR, —SR, —S(O)R, —S(O)₂R, —N(R)₂, —Si(R)₃, or an optionally substituted C₁₋₄ aliphatic; each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a tricyclic ring selected from

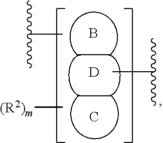

wherein
each of Ring B, Ring C, and Ring D is independently a fused ring selected from 6-membered aryl containing 0-3 nitrogens, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-3 heteroatoms independently selected from nitrogen, oxygen or sulfur; and
m is 0, 1, 2, 3, 4, 5, 6, 7, 8, 9, 10, 11, 12, 13, 14, 15, or 16.

As described above, in certain embodiments, the present invention provides a compound of Formula II-b:

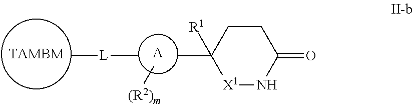

or a pharmaceutically acceptable salt thereof, wherein L and TAMBM are as defined above and described in embodiments herein, wherein:
   X¹ is a bivalent moiety selected from a covalent bond, —CH₂—, —C(O)—, —C(S)—, or

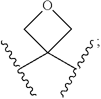 [SMILES: *C1(*)COC1]

R^{l} is hydrogen, deuterium, halogen, —CN, —OR, —SR, —S(O)R, —S(O)₂R, —N(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a tricyclic ring selected from

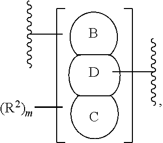

wherein
each of Ring B, Ring C, and Ring D is independently a fused ring selected from 6-membered aryl containing 0-3 nitrogens, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-3 heteroatoms independently selected from nitrogen, oxygen or sulfur; and
m is 0, 1, 2, 3, 4, 5, 6, 7, 8, 9, 10, 11, 12, 13, 14, 15, or 16.

As described above, in certain embodiments, the present invention provides a compound of Formula II-c:

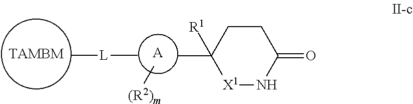

or a pharmaceutically acceptable salt thereof, wherein L and TAMBM are as defined above and described in embodiments herein, wherein:
   X¹ is a bivalent moiety selected from a covalent bond, —CH₂—, —C(O)—, —C(S)—, or

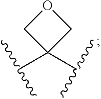 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic; each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a tricyclic ring selected from

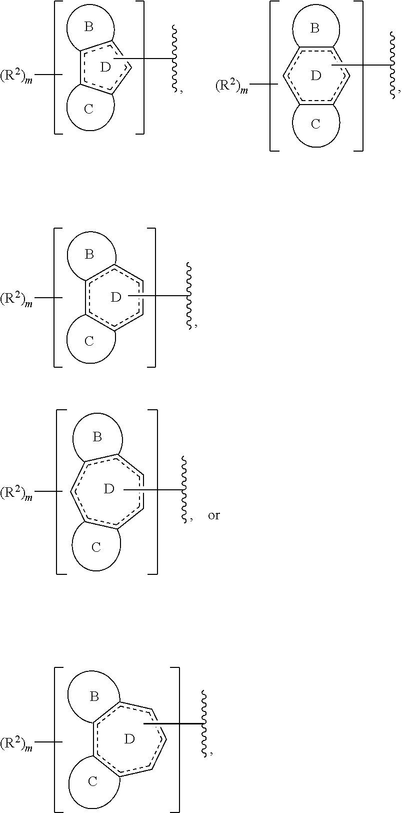

wherein
each of Ring B and Ring C is independently a fused ring selected from 6-membered aryl containing 0-3 nitrogens, 5 to 7-membered saturated or partially unsaturated carbocyclyl, 5 to 7-membered saturated or partially unsaturated heterocyclyl ring with 1-3 heteroatoms independently selected from boron, nitrogen, oxygen or sulfur, or 5-membered heteroaryl with 1-3 heteroatoms independently selected from nitrogen, oxygen or sulfur;
Ring D is a fused ring selected from aryl containing 0-3 nitrogens, saturated or partially unsaturated carbocyclyl, saturated or partially unsaturated heterocyclyl ring with 1-2 heteroatoms independently selected from nitrogen, oxygen, silicon, or sulfur, or heteroaryl with 1-3 heteroatoms independently selected from nitrogen, oxygen or sulfur;
[symbol] is a single or double bond;
m is 0, 1, 2, 3, 4, 5, 6, 7, 8, 9, 10, 11, 12, 13, 14, 15, or 16.

As described above, in certain embodiments, the present invention provides a compound of Formula II-d:

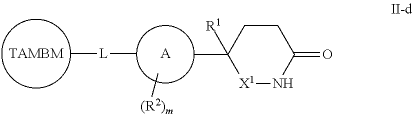

or a pharmaceutically acceptable salt thereof, wherein L and TAMBM are as defined above and described in embodiments herein, wherein:
   X¹ is a bivalent moiety selected from a covalent bond, —CH₂—, —C(O)—, —C(S)—, or

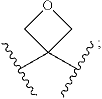 [SMILES: *C1(*)COC1]

R¹ is hydrogen, deuterium, halogen, —CN, —OR, —SR, —S(O)R, —S(O)₂R, —N(R)₂, —Si(R)₃, or an optionally substituted C₁₋₄ aliphatic; each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
Ring A is a tricyclic ring selected from

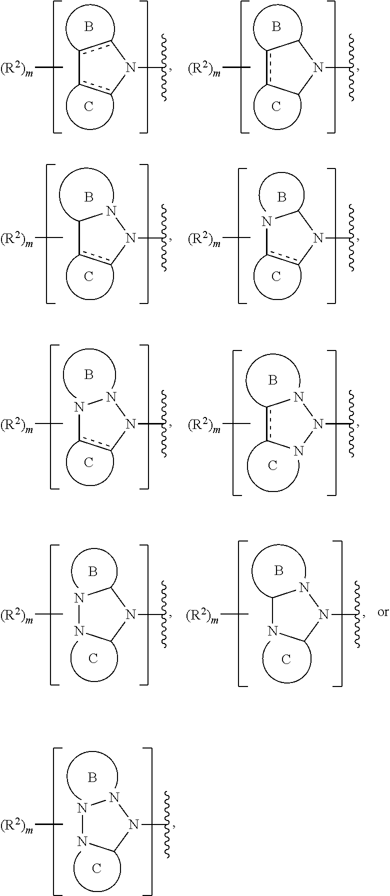

wherein
each of Ring B and Ring C is independently a fused ring selected from 6-membered aryl containing 0-2 nitrogens, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-3 heteroatoms independently selected from nitrogen, oxygen or sulfur;
[symbol] is a single or double bond; and
m is 0, 1, 2, 3, 4, 5, 6, 7, or 8.

In certain embodiments, the present invention provides a compound of Formula I, wherein LBM is an E3 ubiquitin ligase (cereblon) binding moiety thereby forming a compound of formula II-e:

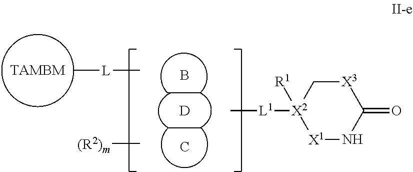

or a pharmaceutically acceptable salt thereof, wherein L and TAMBM are as defined above and described in embodiments herein, and wherein:
   X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or

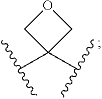 [SMILES: *C1(*)COC1]

X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
   two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur; each of Ring B, Ring D, and Ring C is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—; and
m is 0, 1, 2, 3, 4, 5, 6, 7, 8, 9, 10, 11, 12, 13, 14, 15, or 16.

Where a point of attachment of

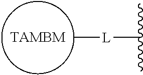

is depicted on Ring B, Ring D, or Ring C, it is intended, and one of ordinary skill in the art would appreciate, that the point of attachment of

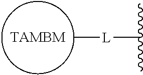

may be on any available carbon or nitrogen atom on Ring B, Ring D, or Ring C, including the ring to which Ring B or Ring C is fused to Ring D.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring B, Ring D, or Ring C including the carbon atom to which Ring B or Ring C are fused to Ring D.

Where a point of attachment of

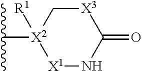

is depicted on Ring B, Ring D, or Ring C, it is intended, and one of ordinary skill in the art would appreciate, that the point of attachment of

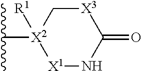

may be on any available carbon or nitrogen atom on Ring B, Ring D, or Ring C, including the carbon atom to which Ring B or Ring C are fused to Ring D.

In some embodiments, a compound of formula II-e above is provided as a compound of formula II-e′ or formula II-e″:

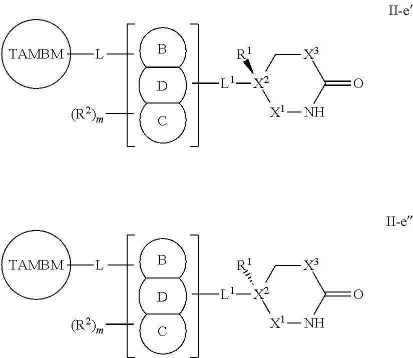

or a pharmaceutically acceptable salt thereof, wherein:
each of TAMBM, Ring E, Ring F, Ring G, L, L¹, R¹, R², X¹, X², X³, and m is as defined above.

In certain embodiments, the present invention provides a compound of Formula I, wherein LBM is an E3 ubiquitin ligase (cereblon) binding moiety thereby forming a compound of formula II-f-1 or II-f-2:

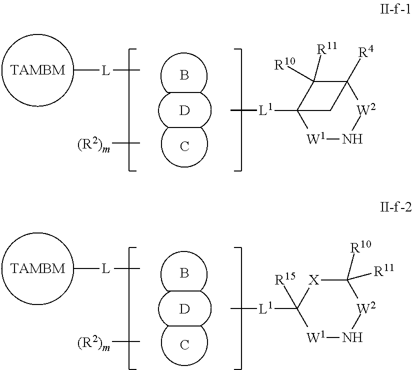

or a pharmaceutically acceptable salt thereof, wherein L and TAMBM are as defined above and described in embodiments herein, and wherein:
   each R² is independently hydrogen, deuterium, —R³, halogen, —CN, —NO₂, —OR, —SR, —NR₂, —SiR₃, —S(O)₂R, —S(O)₂NR₂, —S(O)R, —C(O)R, —C(O)OR, —C(O)NR₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)NR₂, —OP(O)(NR₂)₂—, —N(R)C(O)OR, —N(R)C(O)R, —N(R)C(O)NR₂, —N(R)S(O)₂R, —NP(O)R₂, —N(R)P(O)(OR)₂, —N(R)P(O)(OR)NR₂, —N(R)P(O)(NR₂)₂, or —N(R)S(O)₂R;
   each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
   each of Ring B, Ring D, and Ring C is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-4 heteroatoms independently selected from nitrogen, oxygen or sulfur;
   each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
      two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
   L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S—, —S(O)₂— or —(C)═CH—;
   m is 0, 1, 2, 3, 4, 5, 6, 7, 8, 9, 10, 11, 12, 13, 14, 15, or 16; and
   R⁴, R¹⁰, R¹⁵, W¹, W², and X is as defined in WO 2019/099868, the entirety of each of which is herein incorporated by reference.

Where a point of attachment of

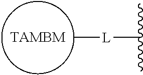

is depicted on Ring B, Ring D, or Ring C, it is intended, and one of ordinary skill in the art would appreciate, that the point of attachment of

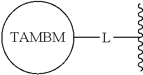

may be on any available carbon or nitrogen atom on Ring B, Ring D, or Ring C, including the ring to which Ring B or Ring C is fused to Ring D.

Where a point of attachment of —(R²)ₘ is depicted on Ring E, Ring F, or Ring G, it is intended, and one of ordinary skill in the art would appreciate, that the point of attachment of —(R²)ₘ may be at any available carbon or nitrogen atom on Ring B, Ring D, or Ring C including the carbon atom to which Ring B or Ring C are fused to Ring D.

Where a point of attachment of

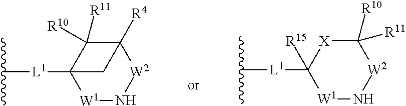

is depicted on Ring B, Ring D, or Ring C, it is intended, and one of ordinary skill in the art would appreciate, that the point of attachment of

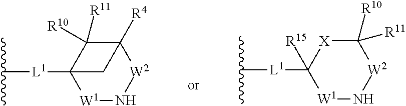

may be on any available carbon or nitrogen atom on Ring B, Ring D, or Ring C, including the carbon atom to which Ring B or Ring C are fused to Ring D.

As defined above and described herein, X¹ is a bivalent moiety selected from a covalent bond, —CH₂—, —C(O)—, —C(S)—, or

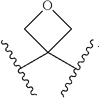 [SMILES: *C1(*)COC1]

In some embodiments, X¹ is a covalent bond. In some embodiments, X¹ is —CH₂—. In some embodiments, X¹ is —C(O)—. In some embodiments, X¹ is —C(S)—. In some embodiments, X¹ is

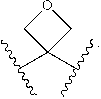 [SMILES: *C1(*)COC1]

In some embodiments, X¹ is selected from those depicted in Table 1, below.

As defined above and described herein, X² is a carbon atom or silicon atom.

In some embodiments, X² is a carbon atom. In some embodiments, X² is a silicon atom.

In some embodiments, X² is selected from those depicted in Table 1, below.

As defined above and described herein, X³ is a bivalent moiety selected from —CH₂— or —Si(R₂)—.

In some embodiments, X³ is —CH₂—. In some embodiments, X² is —Si(R₂)—.

In some embodiments, X³ is selected from those depicted in Table 1, below.

As defined above and described herein, R¹ is hydrogen, deuterium, halogen, —CN, —OR, —SR, —S(O)R, —S(O)₂R, —NR₂, —Si(R₃), or an optionally substituted C₁₋₄ aliphatic.

In some embodiments, R¹ is hydrogen. In some embodiments, R¹ is deuterium. In some embodiments, R¹ is halogen. In some embodiments, R¹ is —CN. In some embodiments, R¹ is —OR. In some embodiments, R¹ is —SR. In some embodiments, R¹ is —S(O)R. In some embodiments, R¹ is —S(O)₂R. In some embodiments, R¹ is —NR₂. In some embodiments, R¹ is —Si(R₃). In some embodiments, R¹ is an optionally substituted C₁₋₄ aliphatic.

In some embodiments, R¹ is selected from those depicted in Table 1, below.

As defined above and described herein, each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or: two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is hydrogen. In some embodiments, R is optionally substituted C₁₋₆ aliphatic. In some embodiments, R is optionally substituted phenyl. In some embodiments, R is optionally substituted 4-7 membered saturated or partially unsaturated heterocyclic having 1-2 heteroatoms independently selected from nitrogen, oxygen, and sulfur. In some embodiments, R is optionally substituted 5-6 membered heteroaryl ring having 1-4 heteroatoms independently selected from nitrogen, oxygen, and sulfur. In some embodiments, two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur.

In some embodiments, R is selected from those depicted in Table 1, below.

As defined above and described herein, each R² is independently hydrogen, —R³, halogen, —CN, —NO₂, —OR, —SR, —N(R)₂, —Si(R₃), —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —N(R)C(O)OR, —N(R)C(O)R, —N(R)C(O)N(R)₂, or —N(R)S(O)₂R.

In some embodiments, R² is hydrogen. In some embodiments, R² is —R³. In some embodiments, R² is halogen. In some embodiments, R² is —CN. In some embodiments, R² is —NO₂. In some embodiments, R² is —OR. In some embodiments, R² is —SR. In some embodiments, R² is —NR₂. In some embodiments, R² is —Si(R₃). In some embodiments, R² is —S(O)₂R. In some embodiments, R² is —S(O)₂NR₂. In some embodiments, R² is —S(O)R. In some embodiments, R² is —C(O)R. In some embodiments, R² is —C(O)OR. In some embodiments, R² is —C(O)NR₂. In some embodiments, R² is —C(O)N(R)OR. In some embodiments, R² is —C(R)₂N(R)C(O)R. In some embodiments, R² is —C(R)₂N(R)C(O)N(R)₂. In some embodiments, R² is —OC(O)R. In some embodiments, R² is —OC(O)NR₂. In some embodiments, R² is —N(R)C(O)OR. In some embodiments, R² is —N(R)C(O)R. In some embodiments, R² is —N(R)C(O)NR₂. In some embodiments, R² is —N(R)S(O)₂R.

In some embodiments, R² is selected from those depicted in Table 1, below.

As defined above and described herein, each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur.

In some embodiments, R³ is an optionally substituted C₁₋₆ aliphatic. In some embodiments, R³ is an optionally substituted phenyl. In some embodiments, R³ is an optionally substituted 4-7 membered saturated or partially unsaturated heterocyclic ring having 1-2 heteroatoms independently selected from nitrogen, oxygen, and sulfur. In some embodiments, R³ is an optionally substituted 5-6 membered heteroaryl ring having 1-4 heteroatoms independently selected from nitrogen, oxygen, and sulfur.

In some embodiments, R³ is selected from those depicted in Table 1, below.

As defined above and described herein, Ring A is a tricyclic ring selected from

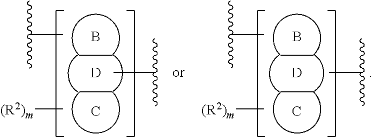

In some embodiments, Ring A is

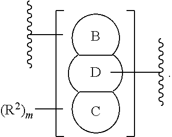

In some embodiments, Ring A is

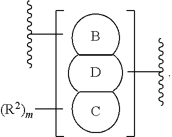

In some embodiments, Ring A is selected from those depicted in Table 1, below.

As defined above and described herein, each of Ring B, Ring C, and Ring D is independently a fused ring selected from 6-membered aryl containing 0-3 nitrogens, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-3 heteroatoms independently selected from nitrogen, oxygen or sulfur.

In some embodiments, each Ring B, Ring C, and Ring D is independently a 6-membered aryl containing 0-2 nitrogen atoms. In some embodiments, each Ring B, Ring C, and Ring D is independently a 5 to 7-membered saturated or partially unsaturated carbocyclyl. In some embodiments, each Ring B, Ring C, and Ring D is independently a 5 to 7-membered saturated or partially unsaturated heterocyclyl with 1-2 heteroatoms independently selected from boron, nitrogen, oxygen, silicon, or sulfur. In some embodiments, each Ring B, Ring C, and Ring D is independently a 5-membered heteroaryl with 1-3 heteroatoms independently selected from nitrogen, oxygen or sulfur.

In some embodiments, Ring B, Ring C, and Ring D is selected from those depicted in Table 1, below.

As defined above and described herein, Ring A is a tricyclic ring selected from

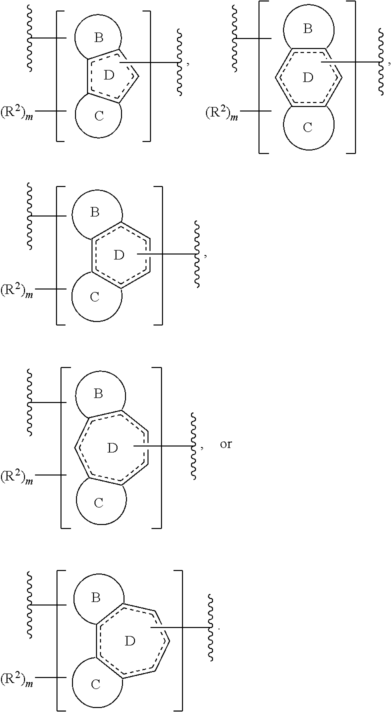

In some embodiments, Ring A is

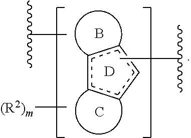

In some embodiments, Ring A is

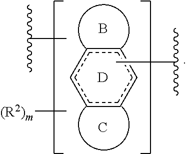

In some embodiment, Ring A is

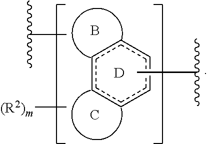

In some embodiments, Ring A is

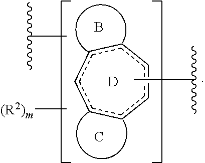

In some embodiments, Ring A is

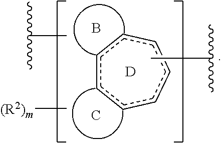

In some embodiments, Ring A is selected from those depicted in Table 1, below.

As defined above and described herein, Ring D is a fused ring selected from aryl containing 0-3 nitrogens, saturated or partially unsaturated carbocyclyl, saturated or partially unsaturated heterocyclyl ring with 1-2 heteroatoms independently selected from nitrogen, oxygen, silicon, or sulfur, or heteroaryl with 1-3 heteroatoms independently selected from nitrogen, oxygen or sulfur.

In some embodiments, Ring D is an aryl containing 0-2 nitrogen atoms. In some embodiments, Ring D is a saturated or partially unsaturated carbocyclyl. In some embodiments, each Ring D is a saturated or partially unsaturated heterocyclyl with 1-2 heteroatoms independently selected from nitrogen, oxygen, silicon, or sulfur. In some embodiments, Ring D is a heteroaryl with 1-3 heteroatoms independently selected from nitrogen, oxygen or sulfur.

In some embodiments, Ring D is

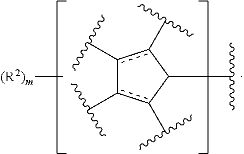

In some embodiments, Ring D is

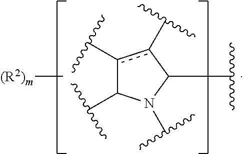

In some embodiments, Ring D is

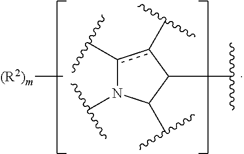

In some embodiments, Ring D is

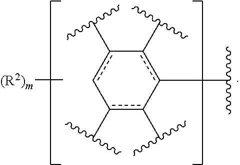

In some embodiments, Ring D is

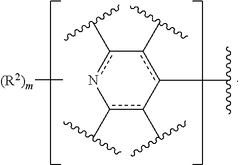

In some embodiments, Ring D is

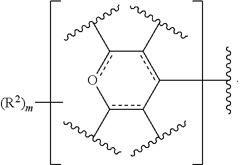

In some embodiments, Ring D is

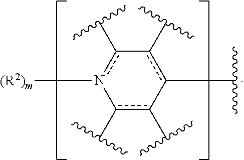

In some embodiments, Ring D is

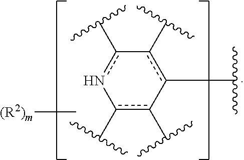

In some embodiments, Ring D is

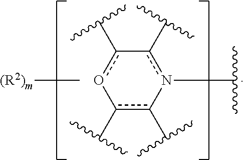

In some embodiments, Ring D is

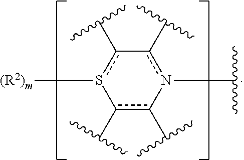

In some embodiments, Ring D is

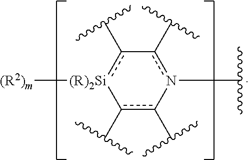

In some embodiments, Ring D is

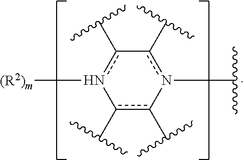

In some embodiments, Ring D is

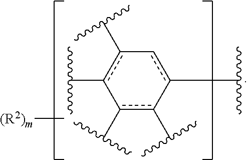

In some embodiments, Ring D is

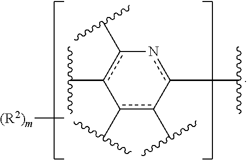

In some embodiments, Ring D is

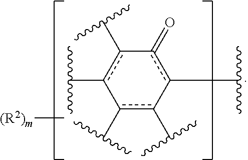

In some embodiments, Ring D is

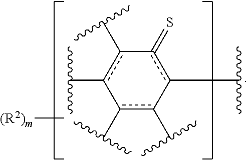

In some embodiments, Ring D is

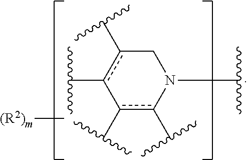

In some embodiments, Ring D is

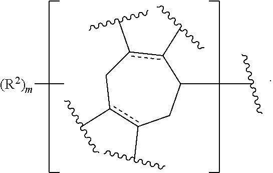

In some embodiments, Ring D is

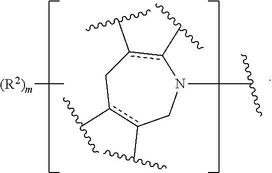

In some embodiments, Ring D is

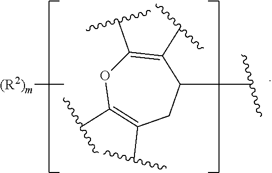

In some embodiments, Ring D is

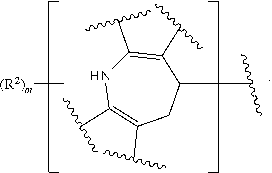

In some embodiments, Ring D is

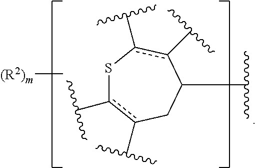

In some embodiments, Ring D is

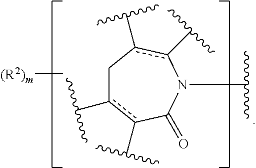

In some
embodiments, Ring D is

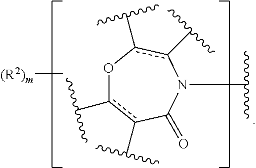

In some embodiments, Ring D is

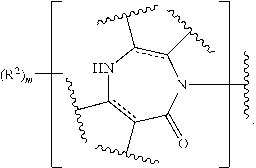

In some embodiments, Ring D is

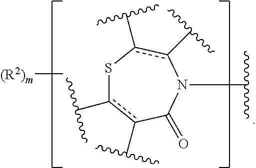

In some embodiments, Ring D is

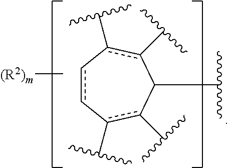

In some embodiments, Ring D is

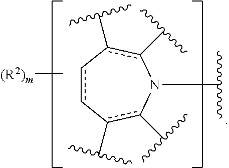

In some embodiments, Ring D is

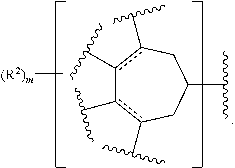

In some embodiments, Ring D is

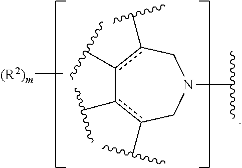

In some embodiments, Ring D is

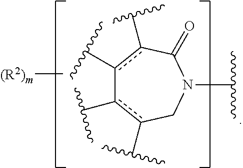

In some embodiments, Ring D is

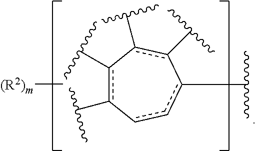

In some embodiments, Ring D is

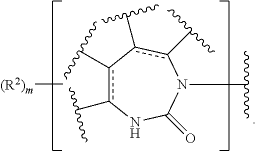

In some embodiments, Ring D is

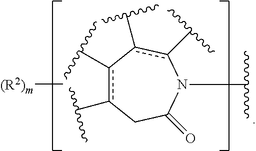

In some embodiments, Ring D is selected from those depicted in Table 1, below.

As defined above and described herein, m is 0, 1, 2, 3, 4, 5, 6, 7, 8, 9, 10, 11, 12, 13, 14, 15, or 16.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8. In some embodiments, m is 9. In some embodiments, m is 10. In some embodiments, m is 11. In some embodiments, m is 12. In some embodiments, m is 13. In some embodiments, m is 14. In some embodiments, m is 15. In some embodiments, m is 16.

In some embodiments, m is selected from those depicted in Table 1, below.

As defined above and described herein, Ring A is a tricyclic ring selected from

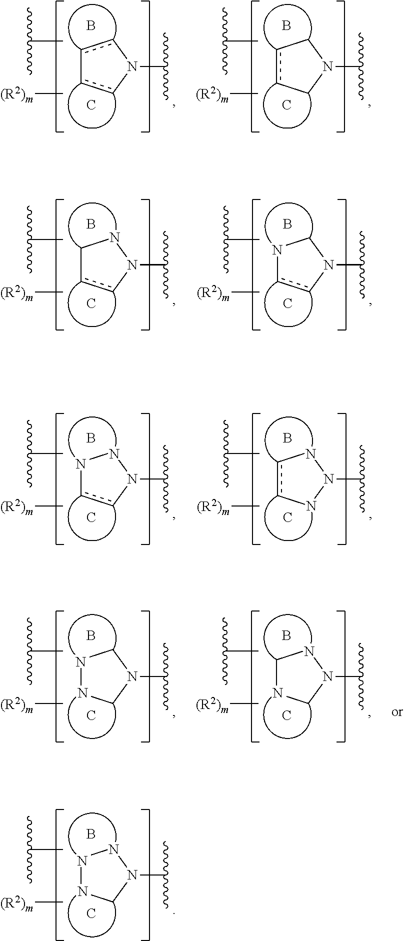

In some embodiments, Ring A is

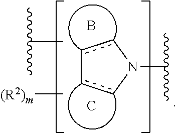

In some embodiments, Ring A is

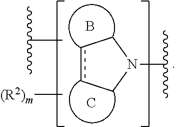

In some embodiment, Ring A is

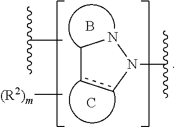

In some embodiments, Ring A is

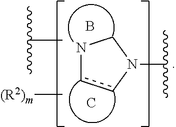

In some embodiments, Ring A is

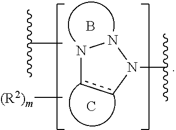

In some embodiments, Ring A is

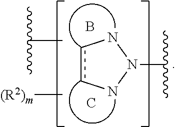

In some embodiments, Ring A is

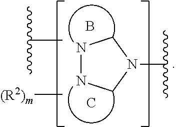

In some embodiments, Ring A is

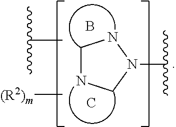

In some embodiments, Ring A is

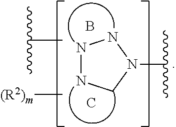

In some embodiments, Ring A is selected from those depicted in Table 1, below.

As defined above and described herein, each Ring B and Ring C is independently a fused ring selected from 6-membered aryl containing 0-2 nitrogen atoms, 5 to 7-membered saturated or partially unsaturated carbocyclyl, 5 to 7-membered saturated or partially unsaturated heterocyclyl with 1-3 heteroatoms independently selected from boron, nitrogen, oxygen, silicon, or sulfur, or 5-membered heteroaryl with 1-3 heteroatoms independently selected from nitrogen, oxygen or sulfur.

In some embodiments, each Ring B and Ring C is independently a 6-membered aryl containing 0-2 nitrogen atoms. In some embodiments, each Ring B and Ring C is independently a 5 to 7-membered saturated or partially unsaturated carbocyclyl. In some embodiments, each Ring B and Ring C is independently a 5 to 7-membered saturated or partially unsaturated heterocyclyl with 1-3 heteroatoms independently selected from boron, nitrogen, oxygen, silicon, or sulfur. In some embodiments, each Ring B and Ring C is independently a 5-membered heteroaryl with 1-3 heteroatoms independently selected from nitrogen, oxygen or sulfur.

In some embodiments, each Ring B and Ring C is independently

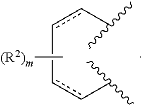

In some embodiments, each Ring B and Ring C is independently

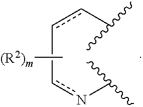

In some embodiments, each Ring B and Ring C is independently

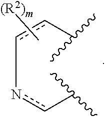

In some embodiments, each Ring B and Ring C is independently

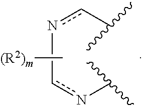

In some embodiments, Ring B and Ring C is independently

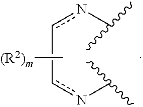

In some embodiments, Ring B and Ring C is independently is

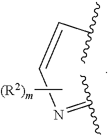

In some embodiments, Ring B and Ring C is independently

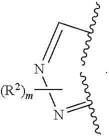

In some embodiments, Ring B and Ring C is independently

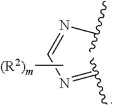

In some embodiments, Ring B and Ring C is independently

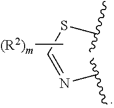

In some embodiments, Ring B and Ring C is independently

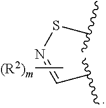

In some embodiments, Ring B and Ring C is independently

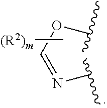

In some embodiments, Ring B and Ring C is independently

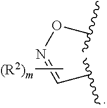

In some embodiments, Ring B and Ring C is independently

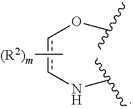

In some embodiments, Ring B and Ring C is independently

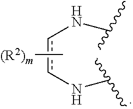

In some embodiments, B and Ring C is independently

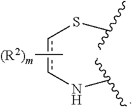

In some embodiments, Ring B and Ring C is independently

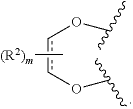

In some embodiments, Ring B and Ring C is independently

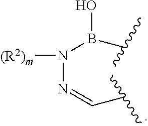

In some embodiments, Ring B and Ring C is independently selected from those depicted in Table 1, below.

As defined above and described here, L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;

In some embodiments, L¹ is a covalent bond. In some embodiments, L¹ is a C₁₋₃ aliphatic. In some embodiments, L¹ is —CH₂—. In some embodiments, L¹ is —C(D)(H)—. In some embodiments, L¹ is —C(D)₂-. In some embodiments, L¹ is —CH₂CH₂—. In some embodiments, L¹ is —NR—. In some embodiments, L¹ is —CH₂NR—. In some embodiments, L¹ is or —O—. In some embodiments, L¹ is —CH₂O—. In some embodiments, L¹ is —S—. In some embodiments, L¹ is —OC(O)—. In some embodiments, L¹ is —C(O)O—. In some embodiments, L¹ is —C(O)—. In some embodiments, L¹ is —S(O)—. In some embodiments, L¹ is —S(O)₂—. In some embodiments, L¹ is —NRS(O)₂—. In some embodiments, L¹ is —S(O)₂NR—. In some embodiments, L¹ is —NRC(O)—. In some embodiments, L¹ is —C(O)NR—.

In some embodiments, Ring L¹ is selected from those depicted in Table 1, below.

As defined above and described herein, [symbol] is a single or double bond

In some embodiments, [symbol] is a single bond. In some embodiments, [symbol] is a double bond.

As defined above and described herein, m is 0, 1, 2, 3, 4, 5, 6, 7, or 8.

In some embodiments, m is 0. In some embodiments, m is 1. In some embodiments, m is 2. In some embodiments, m is 3. In some embodiments, m is 4. In some embodiments, m is 5. In some embodiments, m is 6. In some embodiments, m is 7. In some embodiments, m is 8.

In some embodiments, m is selected from those depicted in Table 1, below.

In certain embodiments, the present invention provides a compound of formula I, wherein LBM is a VHL binding moiety thereby forming a compound of formula II-g-1 or II-g-2:

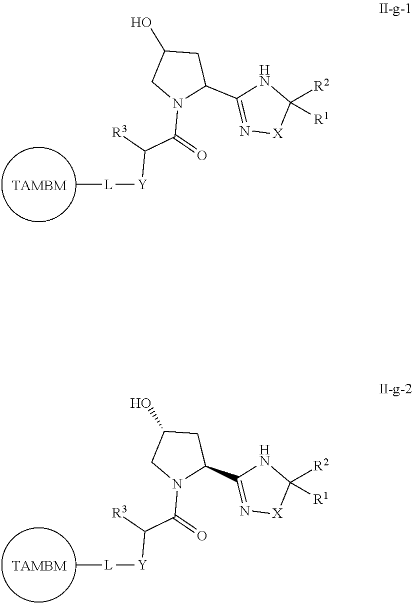

or a pharmaceutically acceptable salt thereof, wherein L and TAMBM are as defined above and described in embodiments herein, and wherein each of the variables R¹, R², R³, X, and Y is as defined and described in WO 2019/084026, the entirety of each of which is herein incorporated by reference.

In certain embodiments, the present invention provides a compound of formula I, wherein LBM is a VHL binding moiety thereby forming a compound of formula II-h-1 or II-h-2:

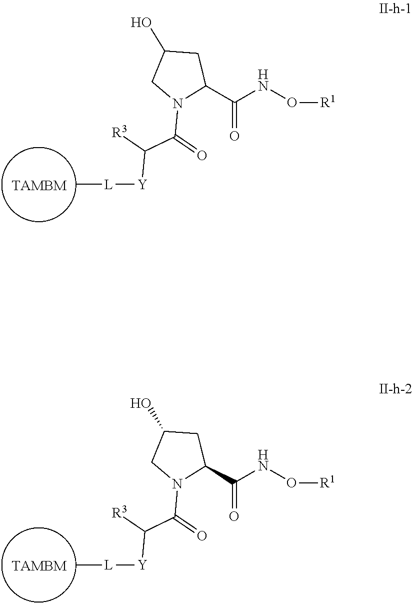

or a pharmaceutically acceptable salt thereof, wherein L and TAMBM are as defined above and described in embodiments herein, and wherein each of the variables R¹, R³, and Y is as defined and described in WO 2019/084030, the entirety of each of which is herein incorporated by reference.

In certain embodiments, the present invention provides a compound of formula I, wherein LBM is a E3 ubiquitin ligase (cereblon) binding moiety thereby forming a compound of formula II-i-1, II-1-2, II-1-3, or II-1-4:

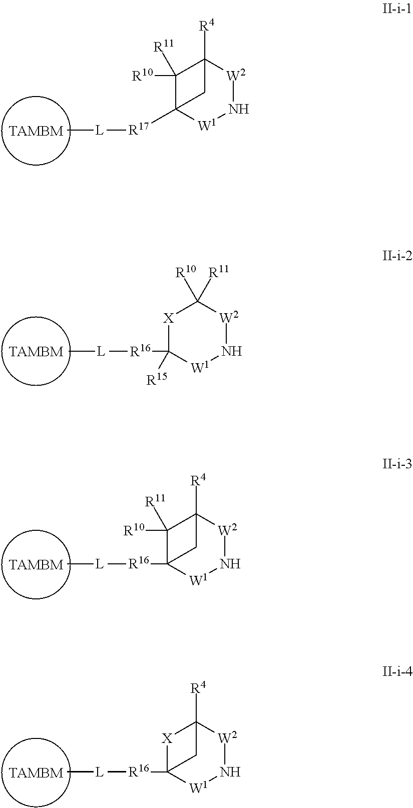

or a pharmaceutically acceptable salt thereof, wherein L and TAMBM are as defined above and described herein, and wherein each of the variables R⁴, R¹⁰, R¹¹, R¹⁵, R¹⁶, R¹⁷, W¹, W², and X is as defined in WO 2019099868 which is herein incorporated by reference in its entirety, and wherein

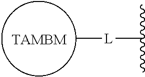

is attached to R¹⁷ or R¹⁶ at the site of attachment of R¹² as defined in WO 2018/237026, such that takes

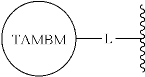

the place of the R¹² substituent.

In certain embodiments, the present invention provides a compound of formula I, wherein LBM is a VHL binding moiety thereby forming a compound of formula II-j:

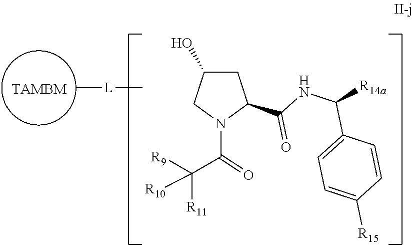

or a pharmaceutically acceptable salt thereof, wherein and TAMBM are as defined above and described in embodiments herein, and wherein each of the variables R₉, R₁₀, R₁₁, R₁₄ₐ, and R₁₅ is as described and defined in WO 2017′030814, WO 2016/118666, and US 2017/0327469, the entirety of each of which is herein incorporated by reference.

In certain embodiments, the present invention provides a compound of formula I, wherein LBM is a VHL binding moiety thereby forming a compound of formula II-k-1 or II-k-2:

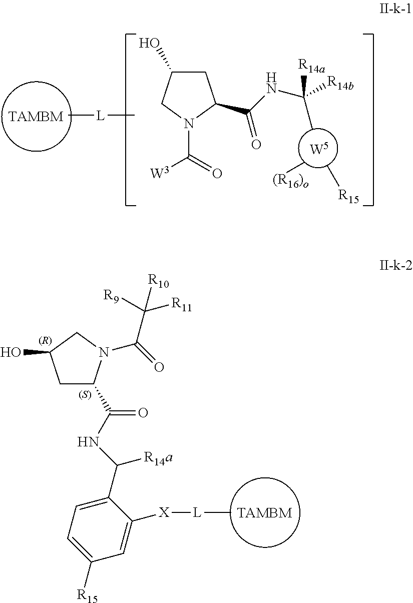

or a pharmaceutically acceptable salt thereof, wherein L and TAMBM are as defined above and described in embodiments herein, and wherein each of the variables X, W, R₉, R₁₀, R₁₁, R₁₄ₐ, and R_{14b}, R₁₅, R¹⁶, and o is as described and defined in WO 2017/030814, WO 20161118666, and US 2017/0327469, the entirety of each of which is herein incorporated by reference.

In certain embodiments, the present invention provides a compound of formula I, wherein LBM is an IAP binding moiety thereby forming a compound of formula II-l:

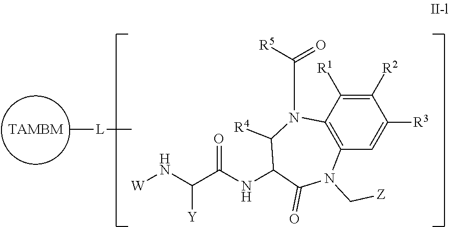

or a pharmaceutically acceptable salt thereof, wherein L and TAMBM are as defined above and described in embodiments herein, and wherein each of the variables W, Y, Z, R¹, R², R³, R⁴, and R⁵ is as described and defined in WO 2014/044622, US 2015/0225449. WO 2015/071393, and US 2016/0272596, the entirety of each of which is herein incorporated by reference.

In certain embodiments, the present invention provides a compound of formula I, wherein LBM is a MDM2 binding moiety thereby forming a compound of formula II-m:

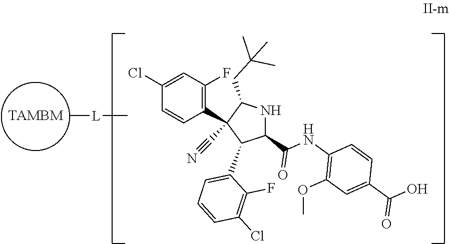

or a pharmaceutically acceptable salt thereof, wherein L and TAMBM are as defined above and described in embodiments herein, as described and defined in Hines, J. et al., Cancer Res. (DOI: 10.1158/0008-5472.CAN-18-2918), the entirety of each of which is herein incorporated by reference.

In certain embodiments, the present invention provides a compound of formula I, wherein LBM is a DCAF16 binding moiety thereby forming a compound of formula II-n:

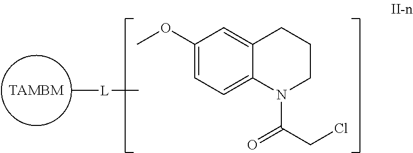

or a pharmaceutically acceptable salt thereof, wherein L and TAMBM are as defined above and described in embodiments herein, as described and defined in Zhang, X. et al., bioRxiv (doi: https://doi.org/10.1101/443804), the entirety of each of which is herein incorporated by reference.

In certain embodiments, the present invention provides a compound of formula I, wherein LBM is a RNF114 binding moiety thereby forming a compound of formula II-o:

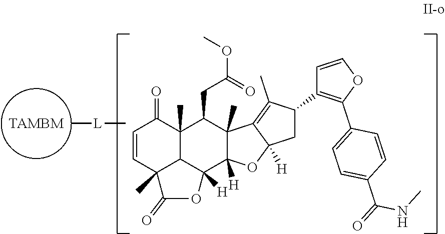

or a pharmaceutically acceptable salt thereof, wherein L and TAMBM are as defined above and described in embodiments herein, as described and defined in Spradin, J. N. et al., bioRxiv (doi: https://doi.org/10.1101/436998), the entirety of each of which is herein incorporated by reference.

In certain embodiments, the present invention provides a compound of formula I, wherein LBM is a RNF4 binding moiety thereby forming a compound of formula II-p:

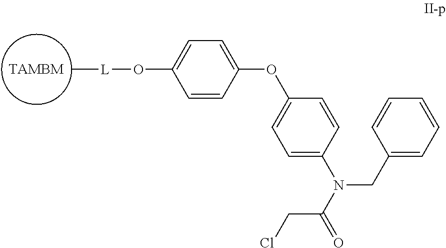

or a pharmaceutically acceptable salt thereof, wherein L and TAMBM are as defined above and described in embodiments herein, as described and defined in Ward, C. C., et al., bioRxiv (doi: https://doi.org/10.1101/439125), the entirety of each of which is herein incorporated by reference.

As defined above and described herein, TAMBM is a TAM receptor kinase binding moiety.

In some embodiments, the present invention provides a compound of Formula III:

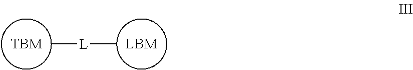

or a pharmaceutically acceptable salt thereof, wherein L and LBM are as described above and herein, and TBM is a TAMBM that preferentially binds Tyro3 (i.e., a Tyro3 binding moiety).

In some embodiments, the present invention provides a compound of Formula IV:

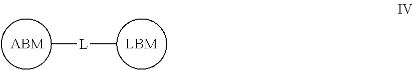

or a pharmaceutically acceptable salt thereof, wherein L and LBM are as described above and herein, and ABM is a TAMBM that preferentially binds Axl (i.e., an Axl binding moiety).

In some embodiments, the present invention provides a compound of Formula V:

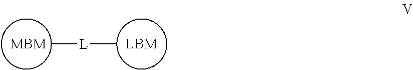

or a pharmaceutically acceptable salt thereof, wherein L and LBM are as described above and herein, and MBM is a TAMBM that preferentially binds MerTK (i.e., a MerTK binding moiety).

In certain embodiments, the present invention provides a compound of Formula VI:

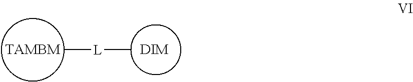

or a pharmaceutically acceptable salt thereof, wherein wherein L and TAMBM are as described above and herein, and DIM is a degradation inducing moiety selected from LBM, lysine mimetic, and hydrogen.

Lysine Mimetic

In some embodiments, DIM is a LBM as described above and herein. In some embodiments, DIM is lysine mimetic. In some embodiments, the covalent attachment of ubiquitin to a member of the TAM receptor kinase family (i.e., Tyro3, Axl, and MerTK) is achieved through the action of a lysine mimetic. In some embodiments, upon TAMBM binding to MerTK, the moiety that mimics a lysine undergoes ubiquitination thereby marking MerTK for degradation via the Ubiquitin-Proteasome Pathway (UPP). In some embodiments, upon TAMBM binding to Axl, the moiety that mimics a lysine undergoes ubiquitination thereby marking Axl for degradation via the Ubiquitin-Proteasome Pathway (UPP). In some embodiments, upon TAMBM binding to Tyro3, the moiety that mimics a lysine undergoes ubiquitination thereby marking Tyro3 for degradation via the Ubiquitin-Proteasome Pathway (UPP).

In some embodiments, DIM is

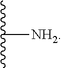 [SMILES: *N]

In some embodiments, DIM is

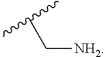 [SMILES: *CN]

In some embodiments, DIM is

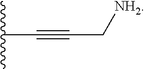 [SMILES: *C#CCN]

In some embodiments, the present invention provides the compound of formula VI wherein DIM is

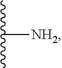 [SMILES: *N]

thereby forming a compound of formula VI-a-1:

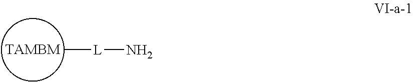

or a pharmaceutically acceptable salt thereof, wherein each of TAMBM and L is as defined above and described in embodiments herein, both singly and in combination.

In some embodiments, the present invention provides the compound of formula I wherein DIM is

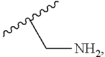 [SMILES: *CN]

thereby forming a compound of formula VI-a-2:

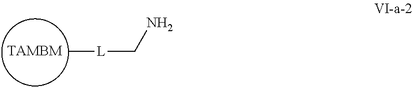

or a pharmaceutically acceptable salt thereof, wherein each of TAMBM and L is as defined above and described in embodiments herein, both singly and in combination.

In some embodiments, the present invention provides the compound of formula I wherein DIM is

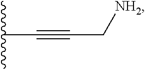 [SMILES: *C#CCN]

thereby forming a compound of formula VI-a-3:

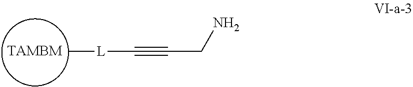

or a pharmaceutically acceptable salt thereof, wherein each of TAMBM and L is as defined above and described in embodiments herein, both singly and in combination.

In certain embodiments, the present invention provides a compound of Formula VI, wherein DIM is a lysine mimetic

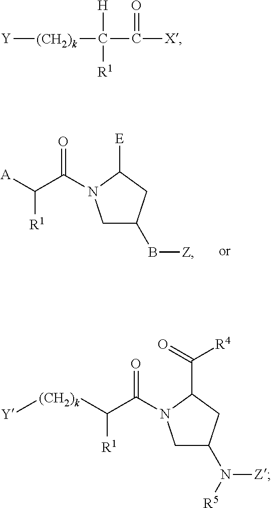

thereby forming a compound of Formulae VI-b-1, VI-b-2, or VI-b-3, respectively:

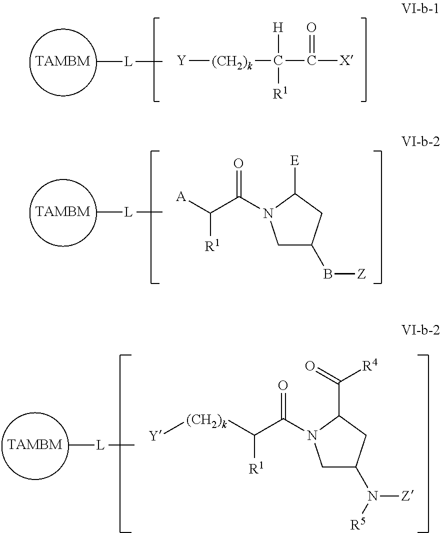

or a pharmaceutically acceptable salt thereof, wherein L and TAMBM are as defined above and described in embodiments herein, and wherein each of the variables R¹, R⁴, R⁵, A, B, E, Y, Y′, Z, Z′, and k are as defined and described in U.S. Pat. No. 7,622,496, the entirety of each of which is herein incorporated by reference.

Hydrogen Atom

In some embodiments, DIM is a hydrogen atom. In some embodiments, the covalent attachment of ubiquitin to a member of the TAM receptor kinase family (i.e., Tyro3, Axl, and MerTK) is achieved through a provided compound wherein DIM is a hydrogen atom. In some embodiments, upon the binding of a provided compound to MerTK, the moiety being hydrogen effectuates ubiquitination thereby marking MerTK for degradation via the Ubiquitin-Proteasome Pathway (UPP). In some embodiments, upon the binding of a provided compound to Axl, the moiety being hydrogen effectuates ubiquitination thereby marking Axl for degradation via the Ubiquitin-Proteasome Pathway (UPP). In some embodiments, upon the binding of a provided compound to Tyro3, the moiety being hydrogen effectuates ubiquitination thereby marking Tyro3 for degradation via the Ubiquitin-Proteasome Pathway (UPP).

In some embodiments, DIM is selected from those depicted in Table 1, below. In some embodiments, the present invention provides the compound of formula VI wherein DIM is a hydrogen atom, thereby forming a compound of formula VI-c:

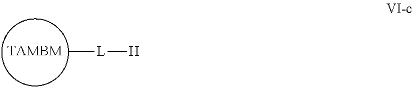

or a pharmaceutically acceptable salt thereof, wherein each of TAMBM and L is as defined above and described in embodiments herein, both singly and in combination.

In certain embodiments, the present invention provides a compound of Formula VI, wherein TAMBM is a pan-TAM binding moiety, binding Tyro3, Axl, and MerTK. In certain embodiments, TAMBM preferentially binds one or two TAM receptor kinases. In some embodiments, TAMBM preferentially binds Tyro3 and Axl relative to MerTK. In some embodiments, TAMBM preferentially binds Tyro3 and MerTK relative to Axl. In some embodiments, TAMBM preferentially binds MerTK and Axl relative to Tyro3. In some embodiments, TAMBM preferentially binds one TAM receptor kinase. In some embodiments, TAMBM is a Tyro3 binding moiety (TBM). In some embodiments, TAMBM is an Axl binding moiety (ABM). In some embodiments, TAMBM is a MerTK binding moiety (MBM).

In certain embodiments, the present invention provides a compound of Formula VI, wherein TAMBM is a TAM receptor kinase inhibitor

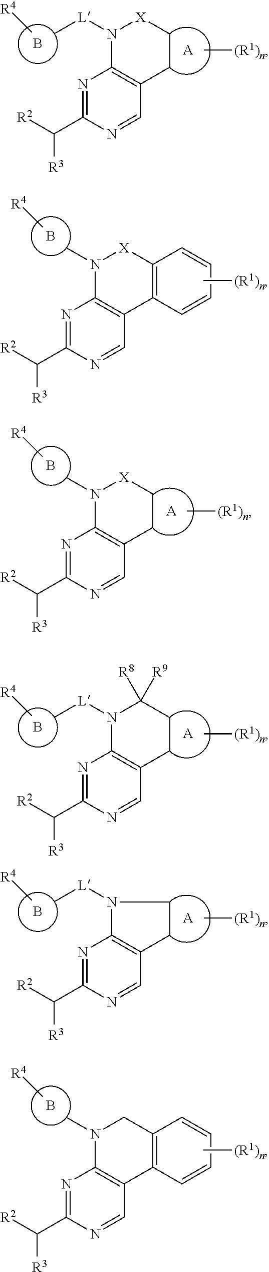

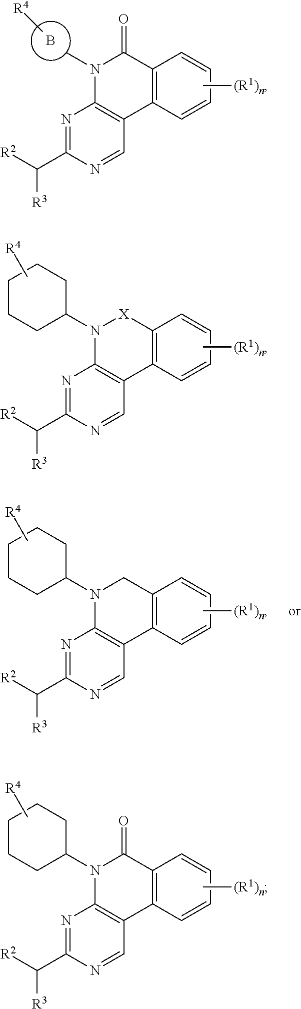

thereby forming a compound of Formulae VI-dd-1, VI-dd-2, VI-dd-3, VI-dd-4, VI-dd-5, VI-dd-6, VI-dd-7, VI-dd-8, V I-dd-9, or VI-dd-10, respectively:

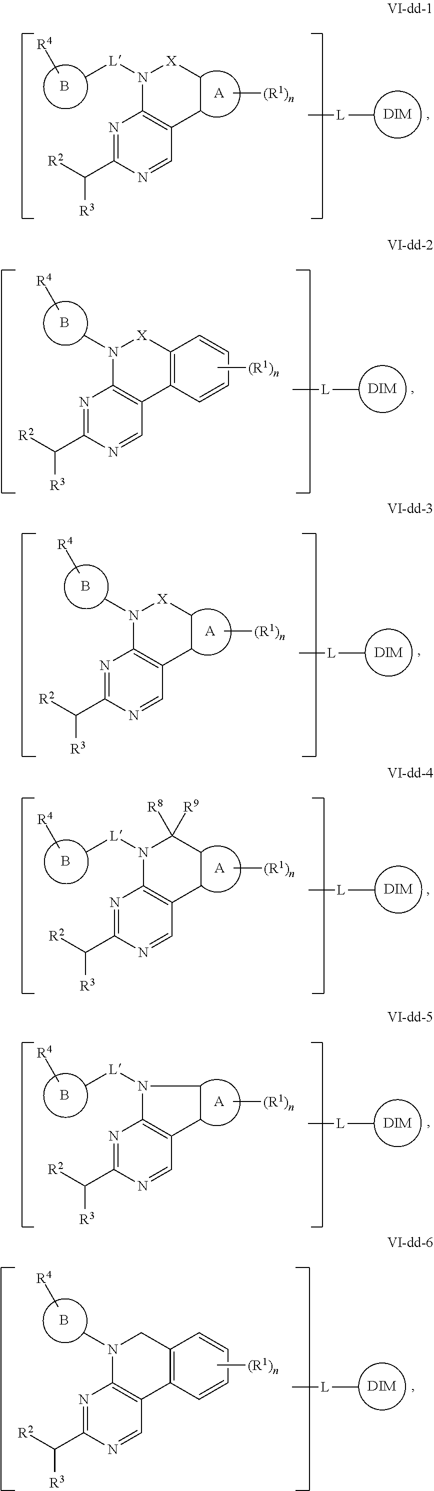

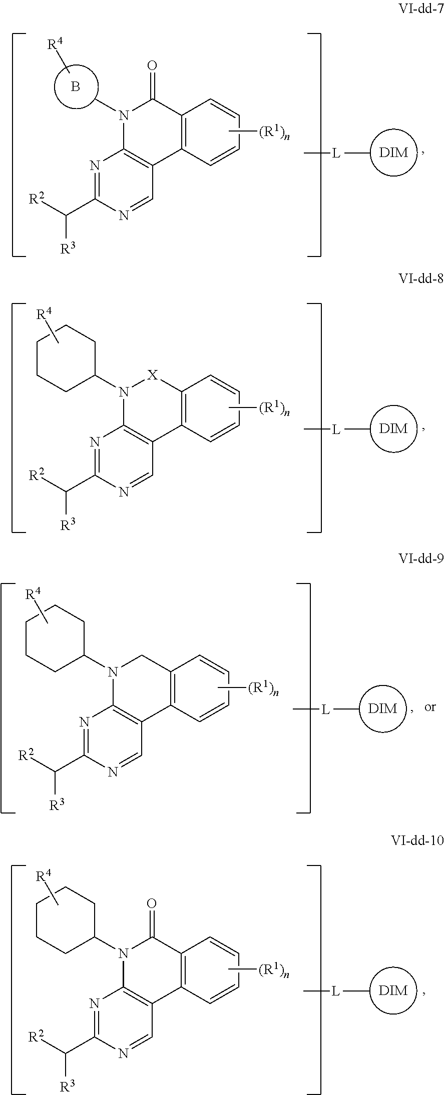

or a pharmaceutically acceptable salt thereof, wherein L and DIM are as defined above and described in embodiments herein, and wherein each of the variables Ring A, Ring B, R¹, R², R³, R⁴, R⁸, R⁹, L′, X, and n are as defined and described in U.S. Pat. No. 9,840,503 and WO 2016/183071, which are incorporated herein, in their entireties.

In certain embodiments, the present invention provides a compound of Formula VI, wherein TAMBM is a TAM receptor kinase inhibitor

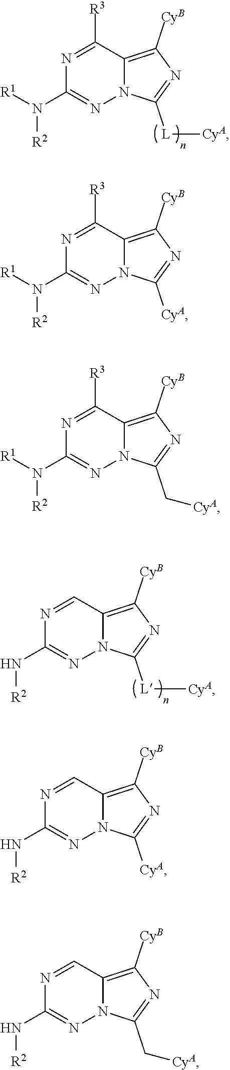

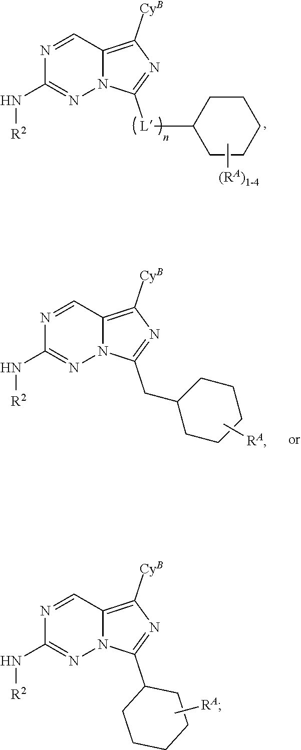

thereby forming a compound of Formulae VI-ee-1, VI-ee-2, VI-ee-3, VI-ee-4, VI-ee-5, VI-ee-6, VI-ee-7, VI-ee-8, or VI-ee-9, respectively:

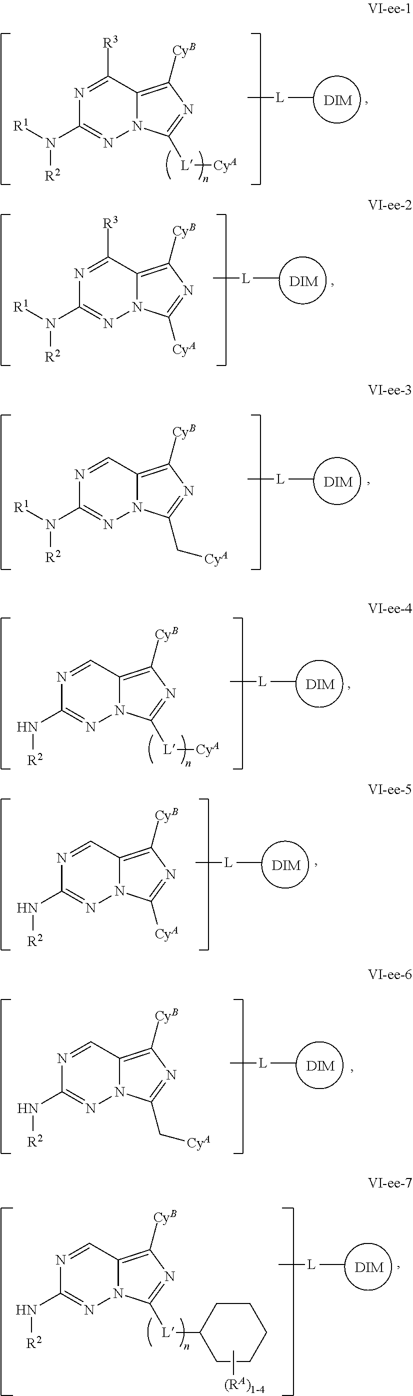

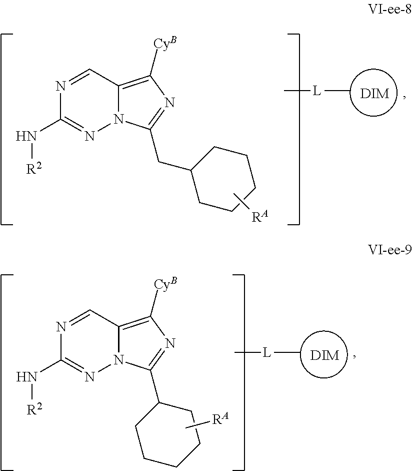

or a pharmaceutically acceptable salt thereof, wherein L and DIM are as defined above and described in embodiments herein, and wherein each of the variables Cy^{A}, Cy^{B}, L′, R¹, R², R³, R^{A}, and n are as defined and described in U.S. Pat. No. 9,708,333, U.S. Pat. App. Pub. No. 2018/0009815, and WO 2017/027717, which are incorporated herein by reference, in their entireties.

In certain embodiments, the present invention provides a compound of Formula VI, wherein TAMBM is a TAM receptor kinase inhibitor

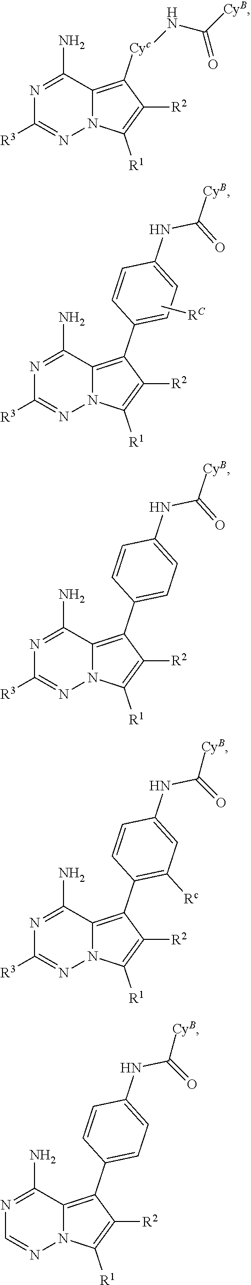

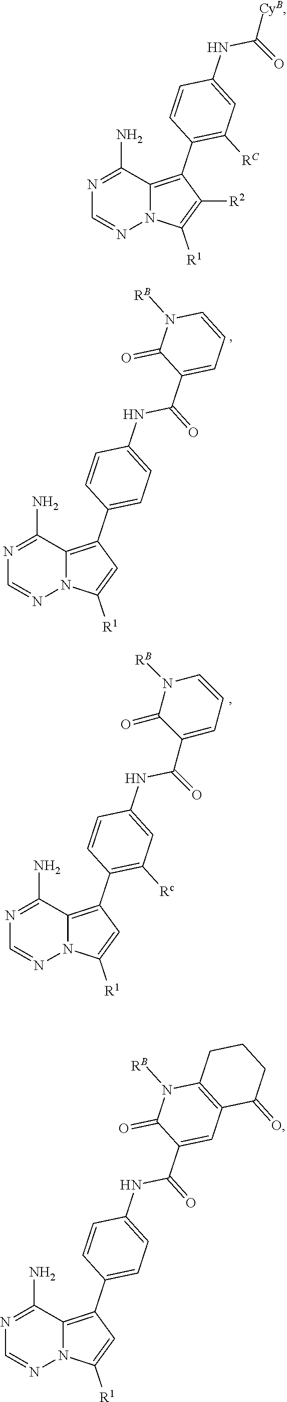

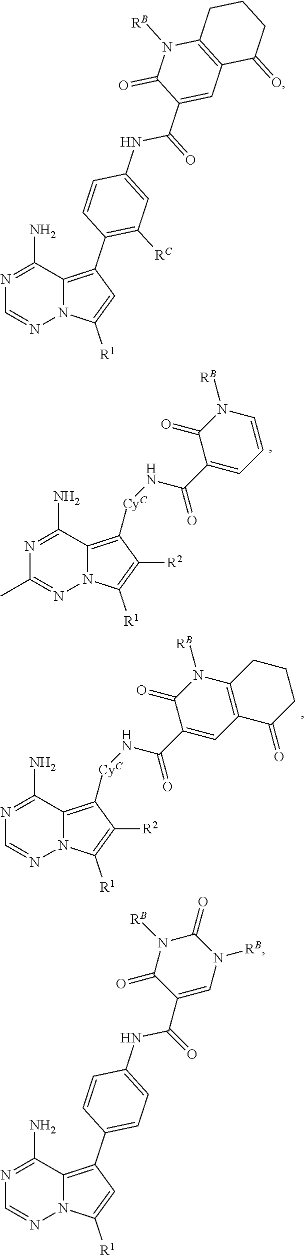

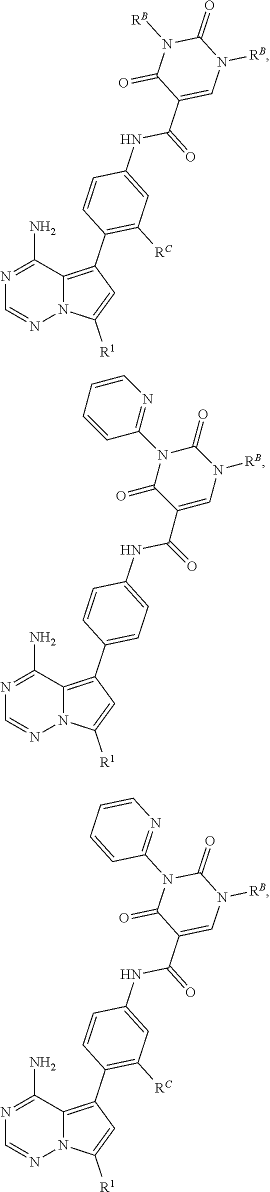

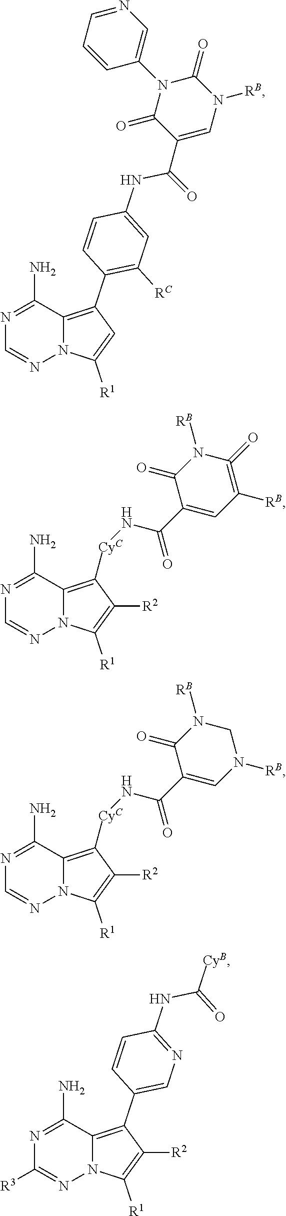

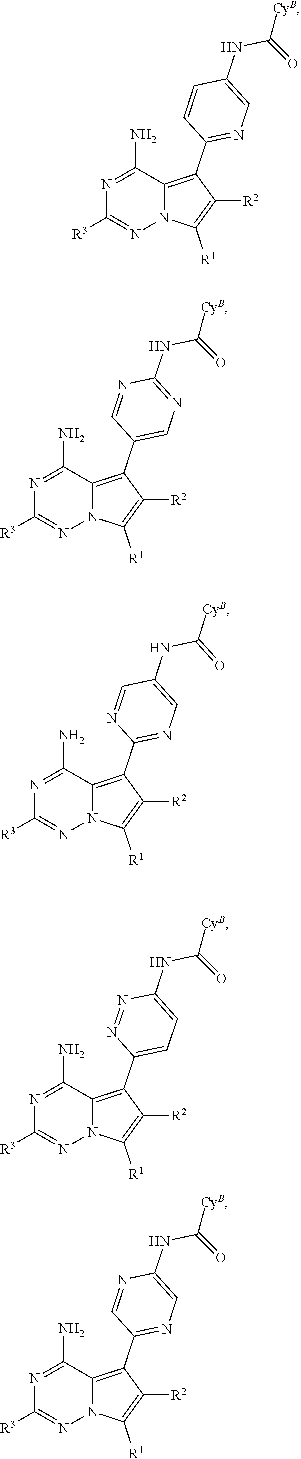

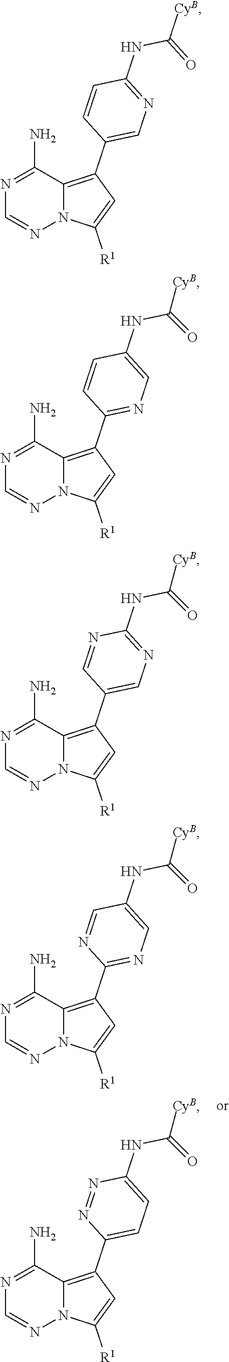

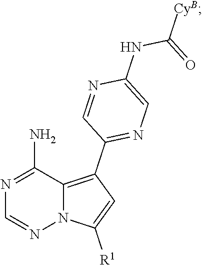

thereby forming a compound of Formulae VI-ff-1, VI-ff-2, VI-ff-3, VI-ff-4, VI-ff-5, VI-ff-6, VI-ff-7, VI-ff-8, VI-ff-9, VI-ff-10, VI-ff-11, VI-ff-12, VI-ff-13, VI-ff-14, VI-ff-15, VI-ff-16, VI-ff-17, VI-ff-18, VI-ff-19, VI-ff-20, VI-ff-21, VI-ff-22, VI-ff-23, VI-ff-24, VI-ff-25, VI-ff-26, VI-ff-27, VI-ff-28, or VI-ff-29, respectively:

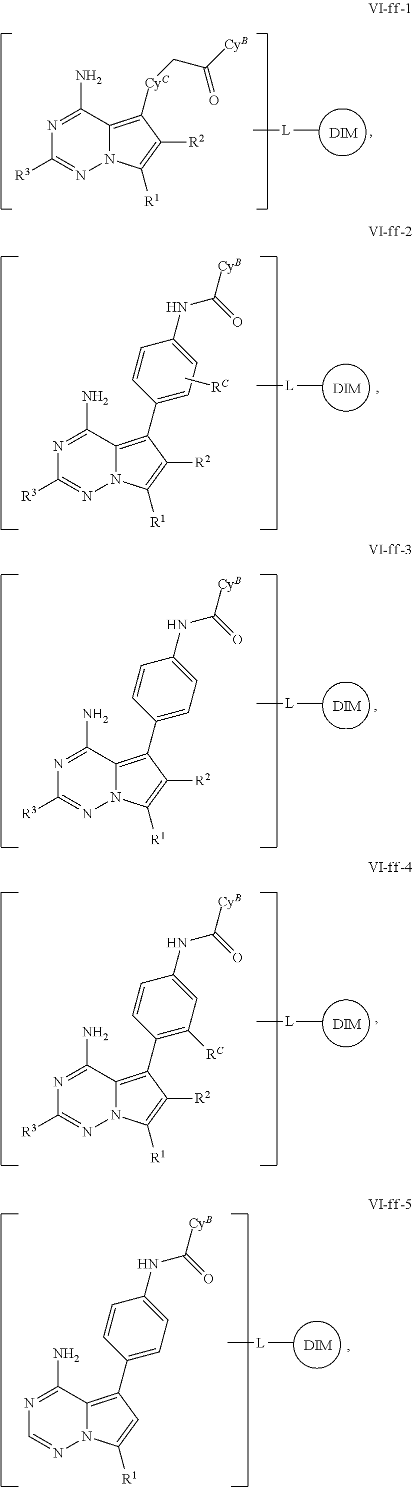

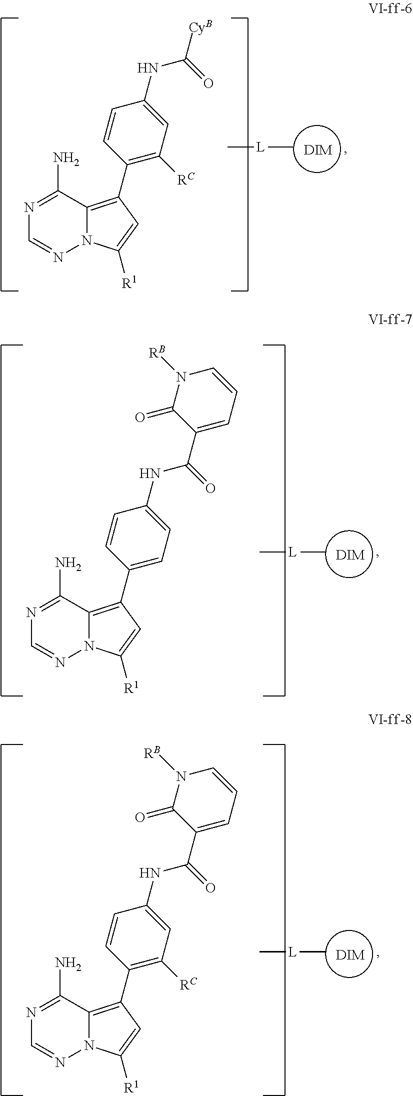

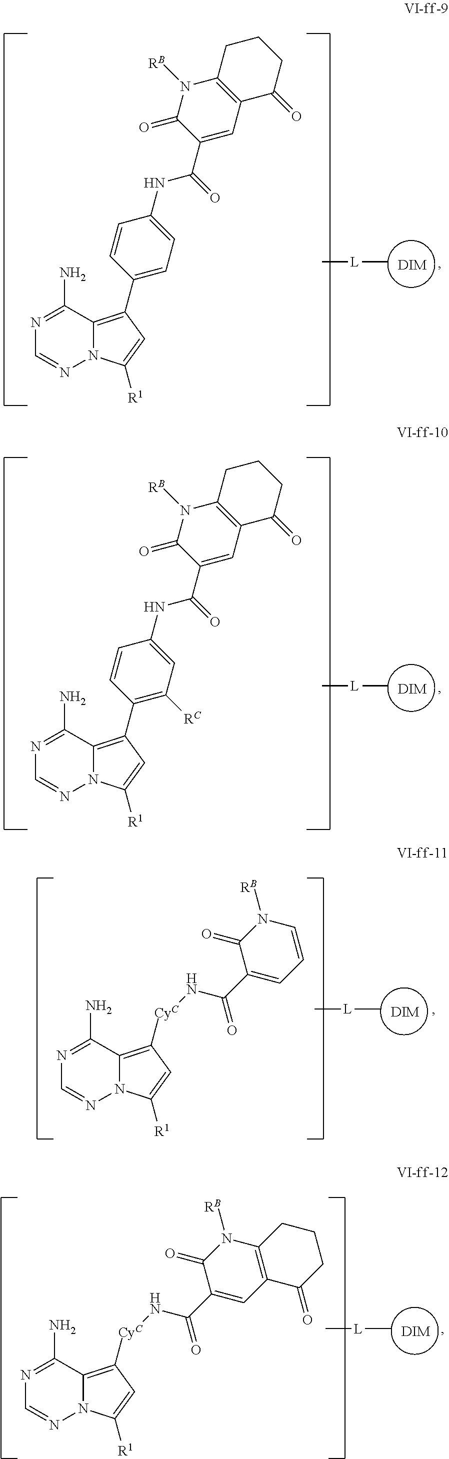

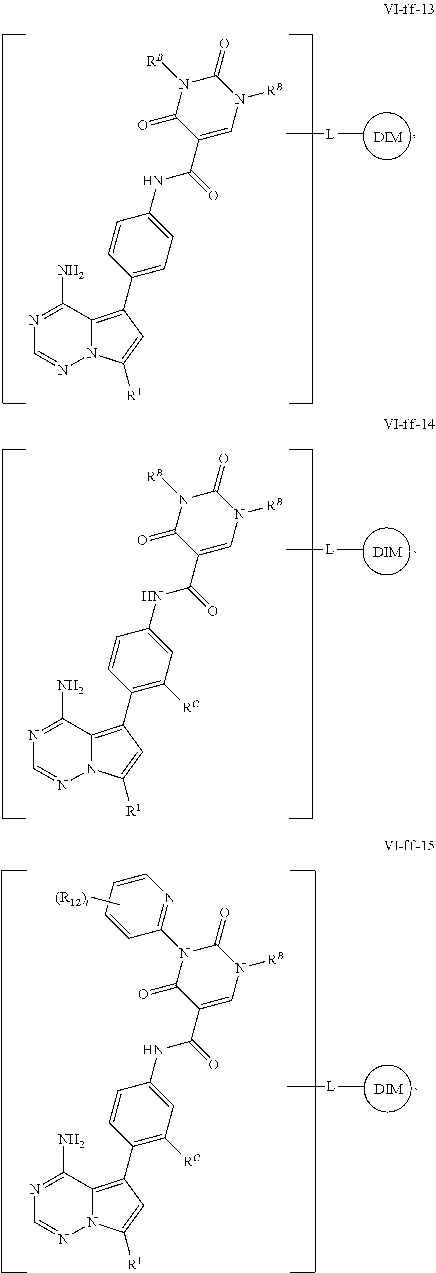

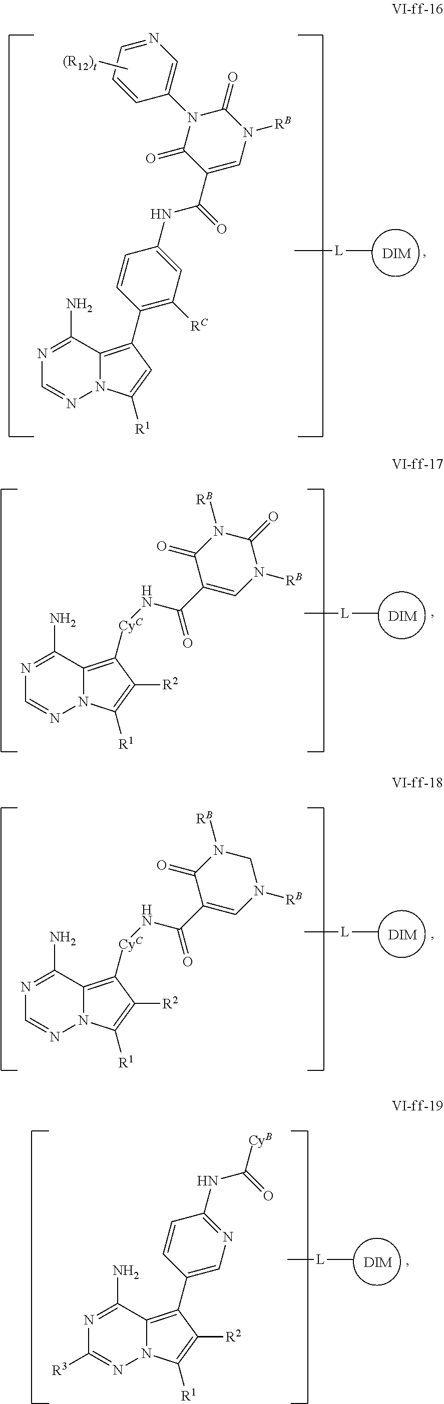

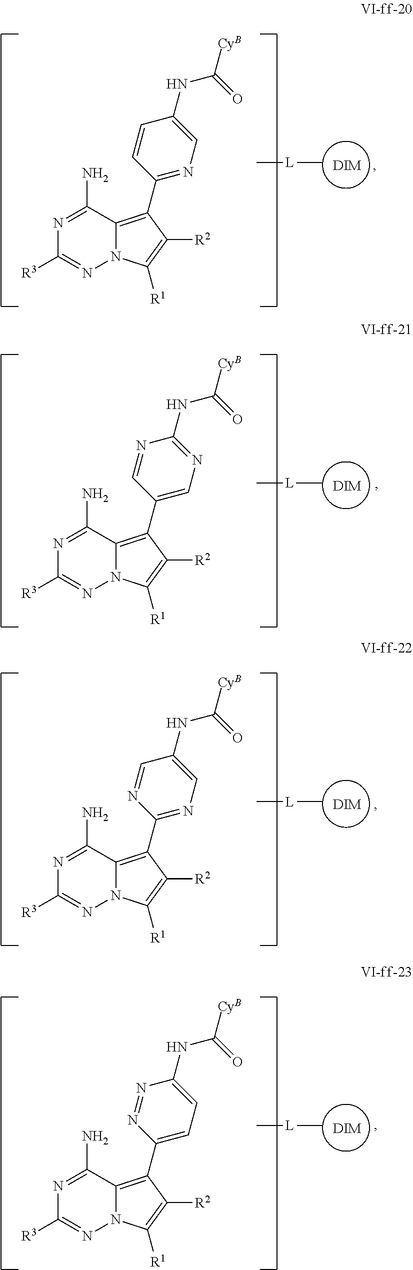

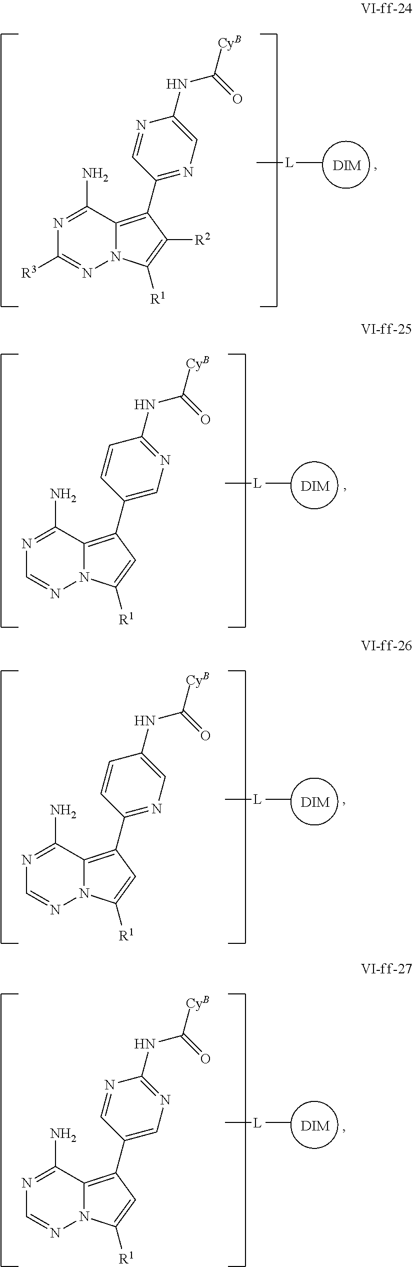

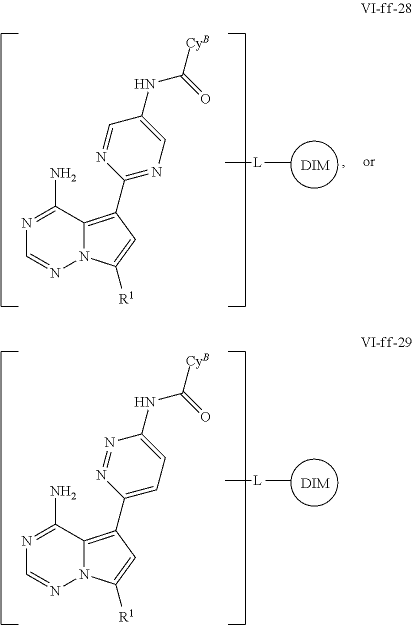

or a pharmaceutically acceptable salt thereof, wherein L and DIM are as defined above and described in embodiments herein, and wherein each of the variables Cy^{B}, Cy^{C}, R¹, R², R³, R^{B}, R^{C}, R₁₂, t, are as defined and described in U.S. Pat. App. Pub. No. 2017/0275290 and U.S. Pat. No. 9,981,975, which are incorporated herein by reference, in their entireties.

In certain embodiments, the present invention provides a compound of Formula I, wherein TAMBM is a TAM receptor kinase inhibitor,

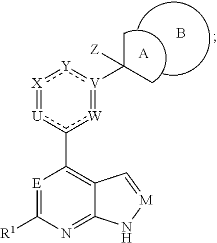

thereby forming a compound of Formula VI-gg:

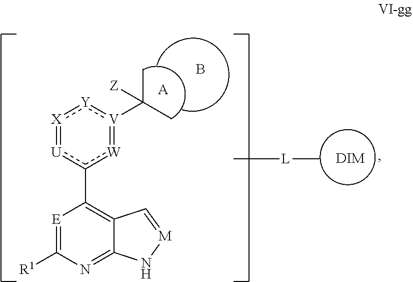

or a pharmaceutically acceptable salt thereof, wherein L and LBM are as defined above and described in embodiments herein, and wherein each of the variables Ring A, Ring B, R¹, E, N, U, V, W, X, Y, Z, M, are as defined and described in WO 2017/035366 and U.S. Pat. App. Pub. No. 2017/0057965, which are incorporated herein by reference, in their entireties.

In certain embodiments, the present invention provides a compound of Formula VI, wherein TAMBM is an TAM kinase receptor inhibitor,

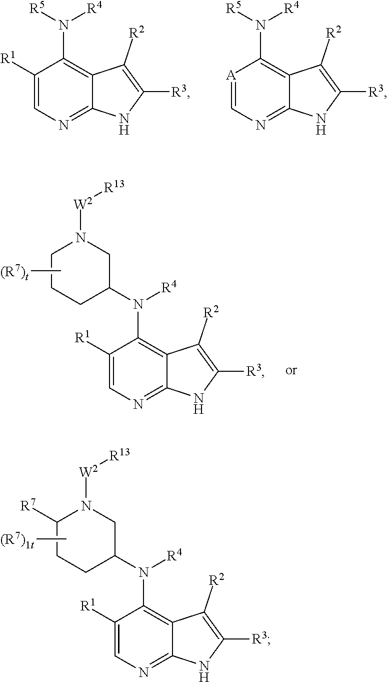

thereby forming a compound of Formulae VI-hh-1, VI-hh-2, VI-hh-3, or VI-hh-4, respectively:

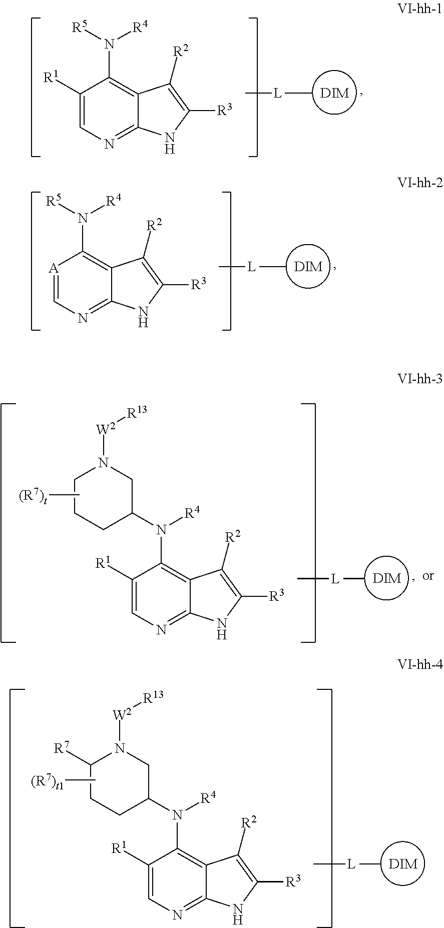

or a pharmaceutically acceptable salt thereof, wherein L and DIM are as defined above and described in embodiments herein, and wherein each of the variables R¹, R², R³, R⁴, R⁵, R⁷, R¹³, A, W², t, and t1, are as defined and described in U.S. Pat. No. 7,335,667, which is incorporated herein by reference, in its entirety.

In certain embodiments, the present invention provides a compound of Formula VI, wherein TAMBM is a TAM receptor kinase inhibitor

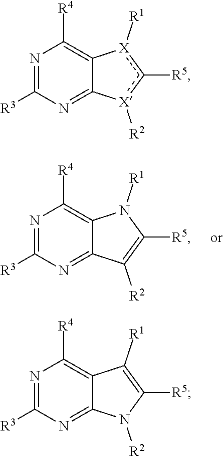

thereby forming a compound of Formulae VI-ii-1, VI-ii-2, or VI-ii-3, respectively:

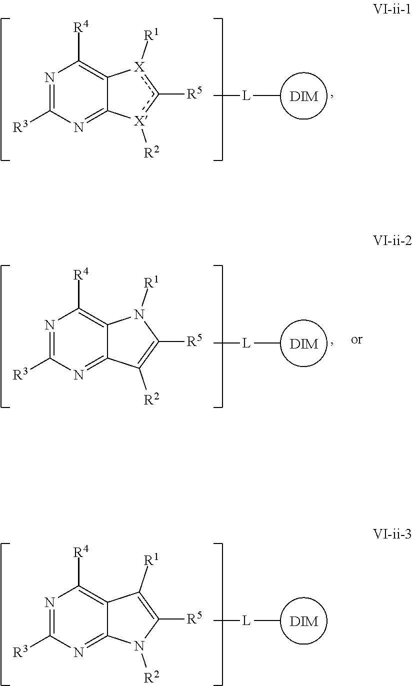

or a pharmaceutically acceptable salt thereof, wherein L and DIM are as defined above and described in embodiments herein, and wherein each of the variables, wherein each of R¹, R², R³, R⁴, R⁵, X and X′ are as defined and described in U.S. Pat. Nos. 9,555,031 and 9,273,056, which are incorporated herein, in their entireties.

In certain embodiments, the present invention provides a compound of Formula VI, wherein TAMBM is a TAM receptor kinase inhibitor

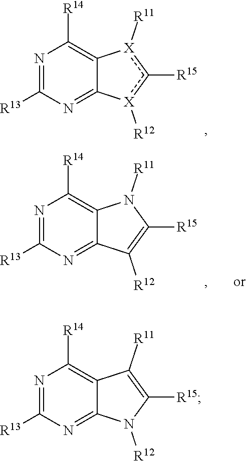

thereby forming a compound of Formulae VI-ii-4, VI-ii-5, or VI-ii-6, respectively:

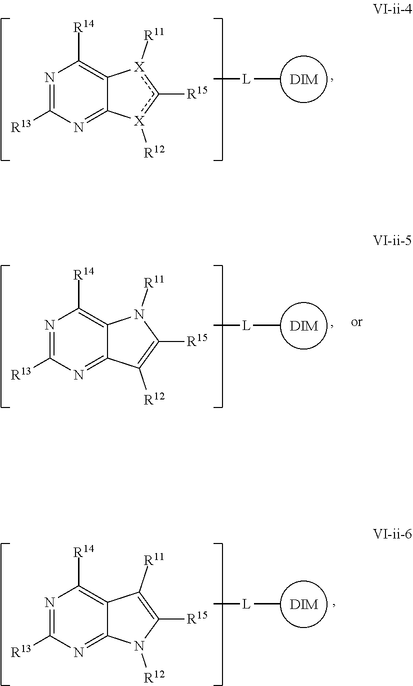

or a pharmaceutically acceptable salt thereof, wherein L and DIM are as defined above and described in embodiments herein, and wherein each of the variables Wherein, each of R¹¹, R¹², R¹³, R¹⁴, R¹⁵, X and X′ are as defined and described in U.S. Pat. Nos. 9,555,031 and 9,273,056, which are incorporated herein, in their entireties.

In certain embodiments, the present invention provides a compound of Formula VI, wherein TAMBM is a TAM receptor kinase inhibitor

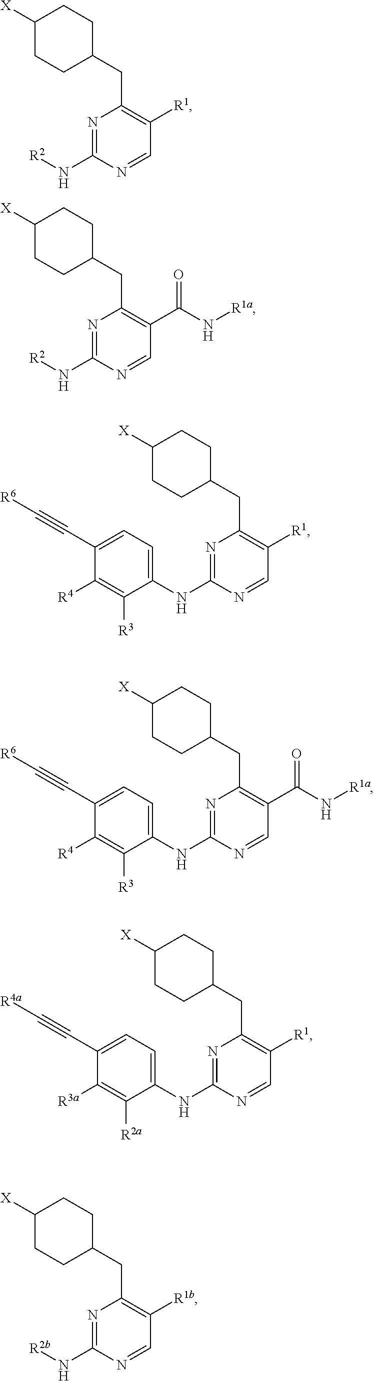

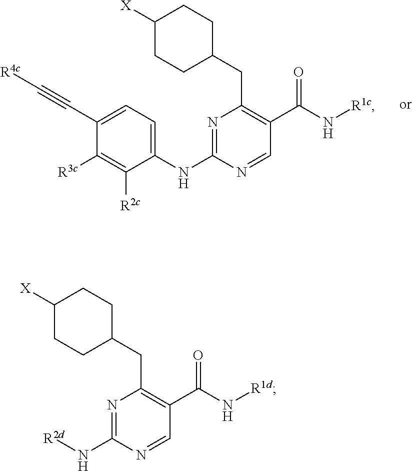

thereby forming a compound of Formulae VI-jj-1, VI-jj-2, VI-jj-3, VI-jj-4, VI-jj-5, VI-jj-6, VI-jj-7, or VI-jj-8, respectively:

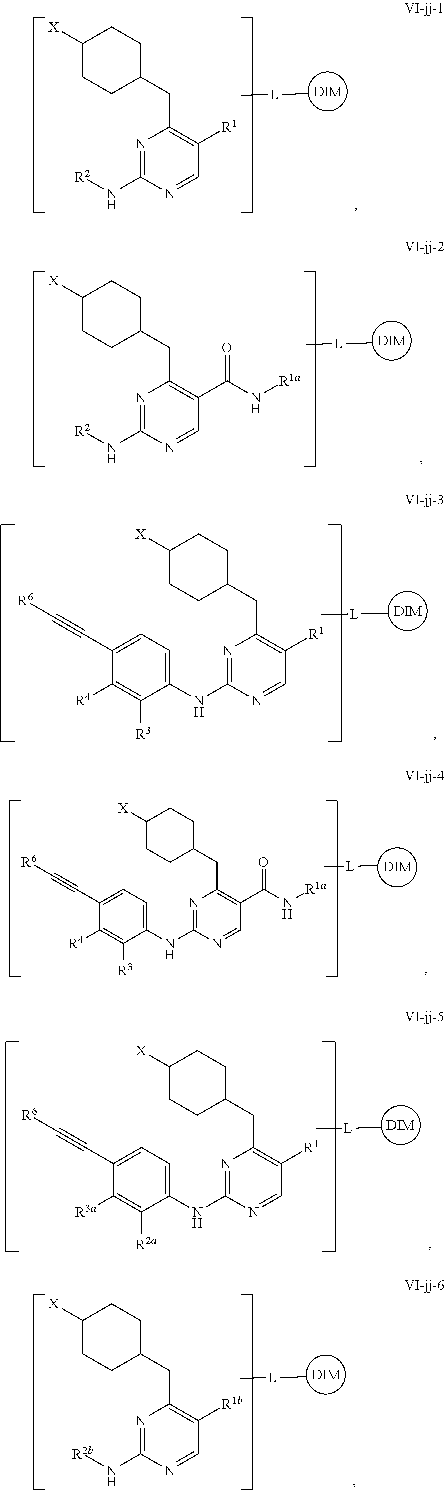

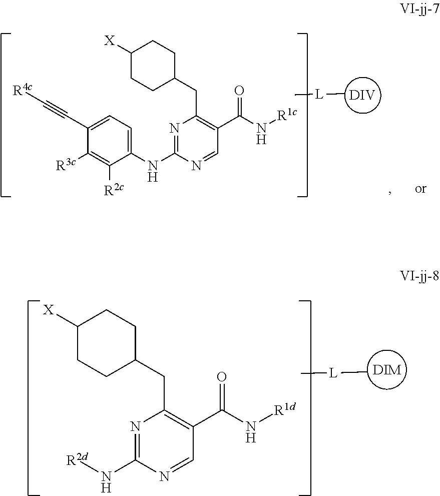

or a pharmaceutically acceptable salt thereof, wherein L and DIM are as defined above and described in embodiments herein, and wherein each of the variables X, R¹, R^{1a}, R^{1b}, R^{1c}, R^{1d}, R₂, R^{2a}, R^{2b}, R^{2c}, R^{2d}, R³, R^{3a}, R^{1c}, R⁴, R^{4a}, R^{4c}, and R⁶are as defined and described in WO 2017/059280 and US 2018/0297977, which is incorporated herein, in its entirety.

In certain embodiments, the present invention provides a compound of Formula VI, wherein TAMBM is a TAM receptor kinase inhibitor

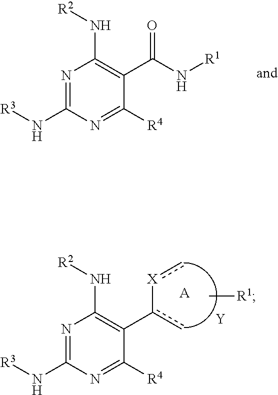

thereby forming a compound of Formulae VI-kk-1 and VI-kk-2, respectively:

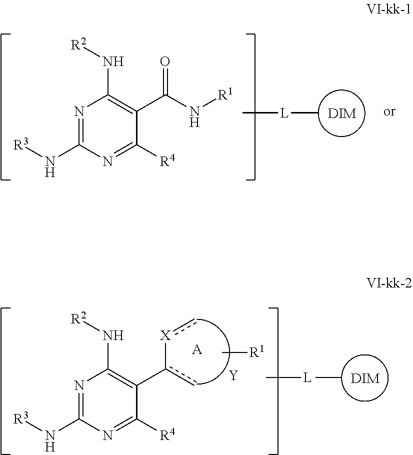

or a pharmaceutically acceptable salt thereof, wherein L and DIM are as defined above and described in embodiments herein, and wherein each of the variables R¹, R², R³, R⁴, Ring A, and Y are as defined and described in U.S. Pat. Nos. 9,567,326 and 9,771,330, which are incorporated herein by reference, in their entireties.

In certain embodiments, the present invention provides a compound of Formula VI, wherein TAMBM is a TAM receptor kinase inhibitor

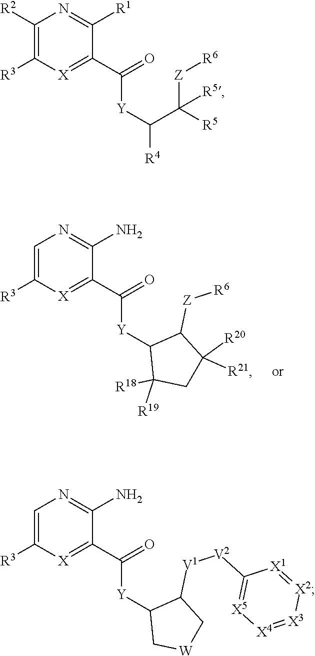

thereby forming a compound of Formulae VI-ll-1, VI-ll-2, or VI-ll-3, respectively:

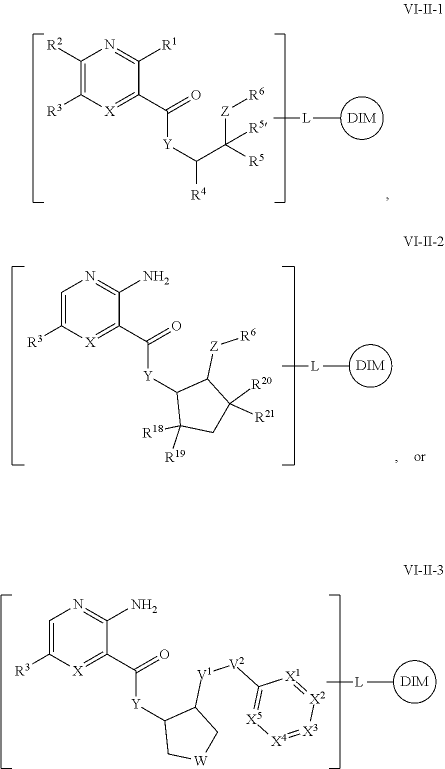

or a pharmaceutically acceptable salt thereof, wherein L and DIM are as defined above and described in embodiments herein, and wherein each of the variables R¹, R², R³, R⁴, R⁵, R^{5′}, R⁶, R¹⁸, R¹⁹, R²⁰, R²¹, V¹, V², X², X³, X⁴, X⁵, Y, and Z, are as defined and described in U.S. Pat. App. Pub. No. 2017/0355690 and WO 2017/039331, which are incorporated herein by reference, in their entireties.

In certain embodiments, the present invention provides a compound of Formula VI, wherein TAMBM is a TAM receptor kinase inhibitor,

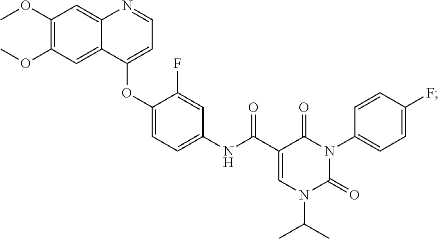 [SMILES: COc1cc2nccc(Oc3ccc(NC(=O)c4cn(C(C)C)c(=O)n(-c5ccc(F)cc5)c4=O)cc3F)c2cc1OC]

thereby forming a compound of Formula VI-mm

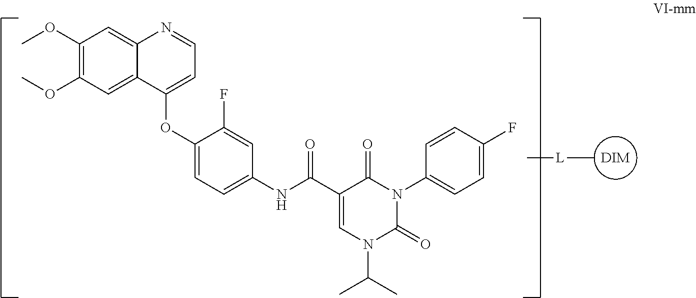

or a pharmaceutically acceptable salt thereof, wherein L and DIM are as defined above and described in embodiments herein, and wherein the TAMBM is as defined and described in WO 2015/017607, which is incorporated herein by reference, in its entirety.

In certain embodiments, the present invention provides a compound of Formula VI, wherein TAMBM is a TAM receptor kinase inhibitor,

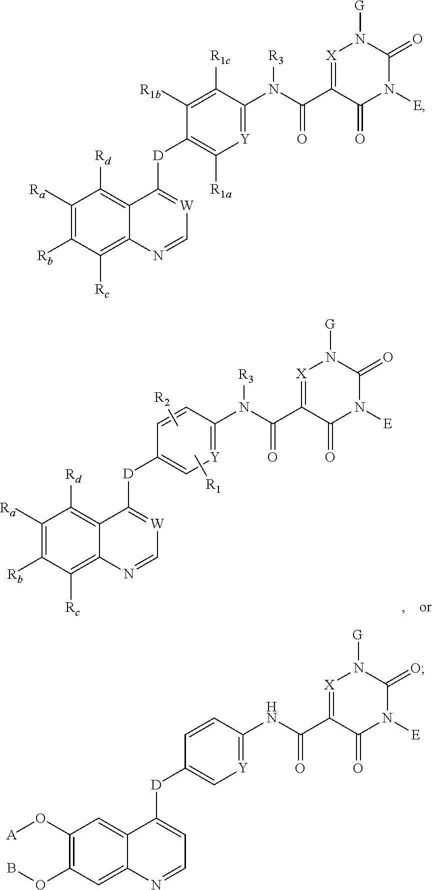

thereby forming a compound of Formulae VI-nn-1, VI-nn-2, or VI-nn-3, respectively:

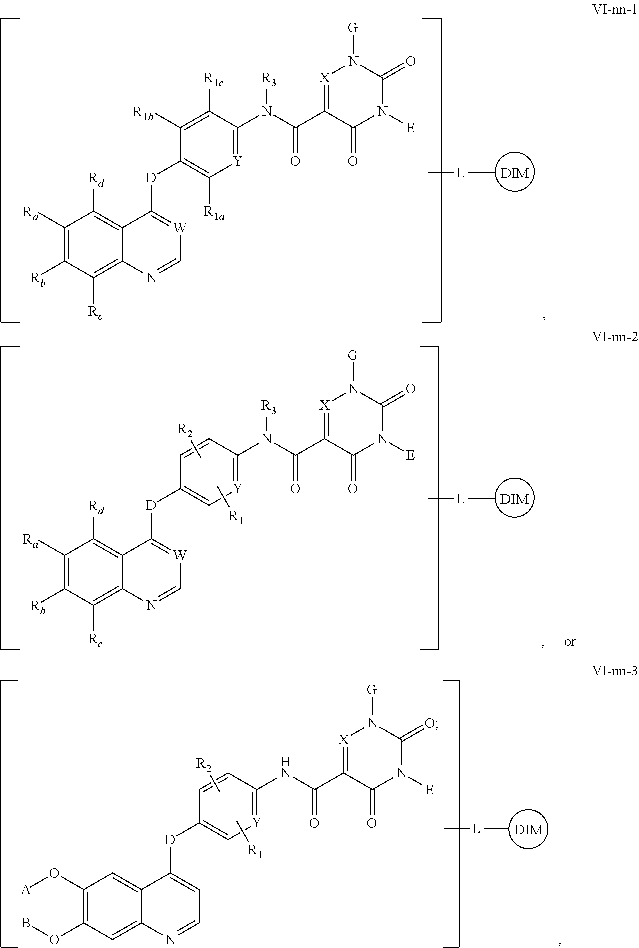

or a pharmaceutically acceptable salt thereof, wherein L and DIM are as defined above and described in embodiments herein, and wherein each of the variables R₁, R₂, R₃, R₁ₐ, R_{1b}, R_{1c}, Rₐ, R_{b}, R_{c}, R_{d}, D, E, G, X, and W, are as defined and described in U.S. Pat. No. 9,120,778, which is incorporated herein by reference, in its entirety.

In some embodiments, TAMBM is selected from a moiety recited in Yan, S. B. et al., LY2801653 is an orally bioavailable multi-kinase inhibitor with potent activity against MET, MST1R, and other oncoproteins, and displays anti-tumor activities in mouse xenograft models, Invest. New Drugs (2013) 31(4): 833-44, such as, for example:

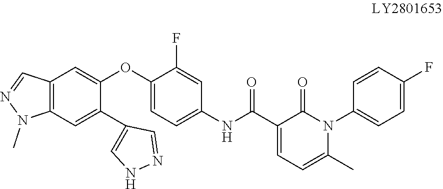 [SMILES: Cc1ccc(C(=O)Nc2ccc(Oc3cc4cnn(C)c4cc3-c3cn[nH]c3)c(F)c2)c(=O)n1-c1ccc(F)cc1]

wherein

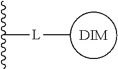

is attached to a modifiable carbon, oxygen, nitrogen or sulfur atom.

In some embodiments, TAMBM is selected from a moiety recited in Knubel, K. H. et al., MerTK inhibition is a novel therapeutic approach for glioblastoma multiforme, Oncotarget (2014) 5(5): 1338-51, such as, for example:

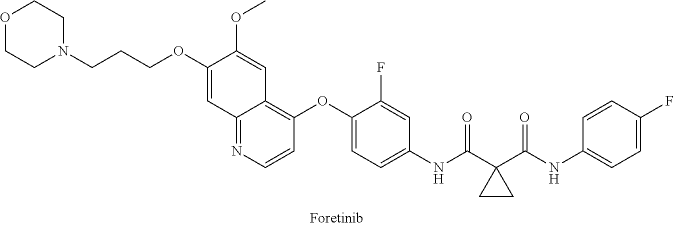 [SMILES: COc1cc2c(Oc3ccc(NC(=O)C4(C(=O)Nc5ccc(F)cc5)CC4)cc3F)ccnc2cc1OCCCN1CCOCC1]

wherein

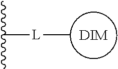

is attached to a modifiable carbon, oxygen, nitrogen or sulfur atom.

In some embodiments, TAMBM is selected from a moiety recited in Page, L. S. et al., MerTK Receptor Tyrosine Kinase Inhibition As a Potential Strategy to Augment Immune-Mediated Clearance of Acute Myeloid Leukemia, Blood (2016) 128(22): 4044-48; and/or Minson, K. A. et al., The MERTK/FLT3 inhibitor MRX-2843 overcomes resistance-conferring FLT3 mutations in acute myeloid leukemia, JCI Insight. (2016) 1(3): e85630, https://doi.org/10.1172/jci.insight.85630, such as, for example:

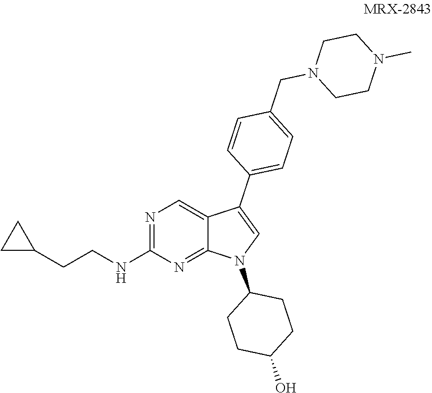 [SMILES: CN1CCN(Cc2ccc(-c3cn([C@H]4CC[C@H](O)CC4)c4nc(NCCC5CC5)ncc34)cc2)CC1]

wherein

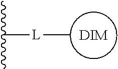

is attached to a modifiable carbon, oxygen, nitrogen or sulfur atom.

In some embodiments, TAMBM is selected from a moiety recited in Kim, J. E. et al., MerTK inhibition by RXDX-106 in MerTK activated gastric cancer cell lines, Oncotarget (2017) 8(62): 105727-34, such as, for example:

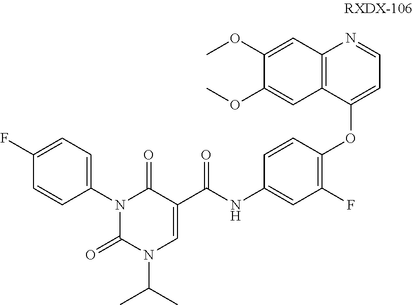 [SMILES: COc1cc2nccc(Oc3ccc(NC(=O)c4cn(C(C)C)c(=O)n(-c5ccc(F)cc5)c4=O)cc3F)c2cc1OC]

wherein

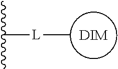

is attached to a modifiable carbon, oxygen, nitrogen or sulfur atom.

In some embodiments, TAMBM is selected from a moiety recited in Zhang, W. et al., UNC2025, a Potent and Orally Bioavailable MER/FLT3 Dual Inhibitor, J. Med. Chem. (2014) 57(16): 7031-41, such as, for example:

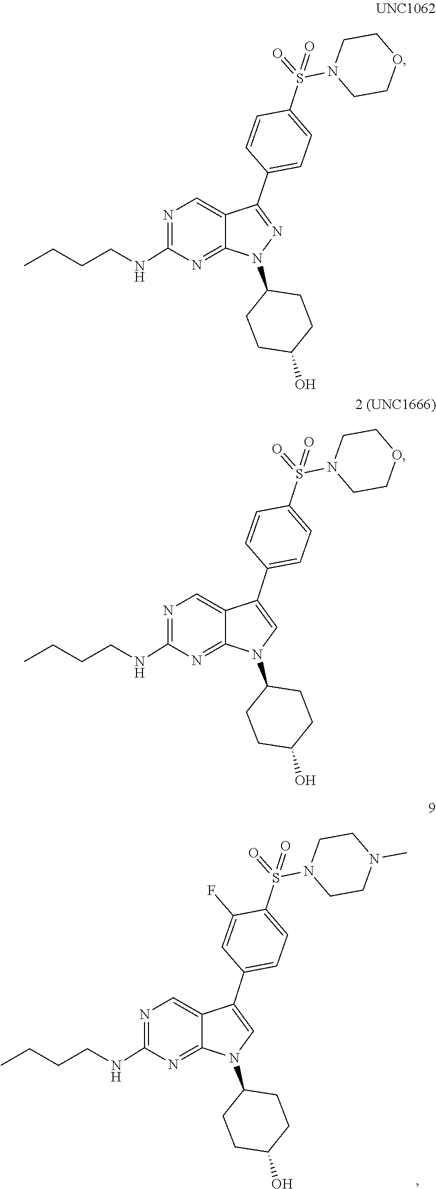 [SMILES: CCCCNc1ncc2c(-c3ccc(S(=O)(=O)N4CCOCC4)cc3)nn([C@H]3CC[C@H](O)CC3)c2n1 ; CCCCNc1ncc2c(-c3ccc(S(=O)(=O)N4CCOCC4)cc3)cn([C@H]3CC[C@H](O)CC3)c2n1 ; CCCCNc1ncc2c(-c3ccc(S(=O)(=O)N4CCN(C)CC4)c(F)c3)cn([C@H]3CC[C@H](O)CC3)c2n1]

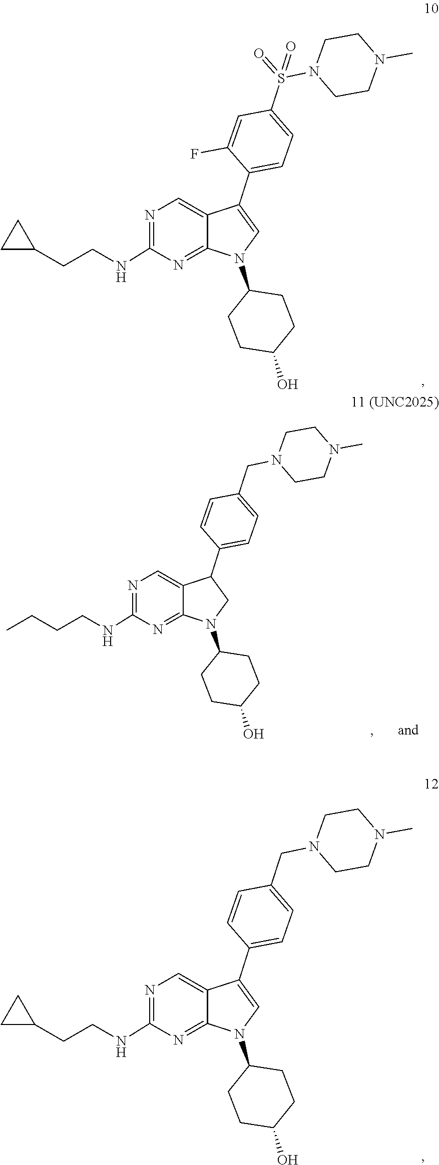

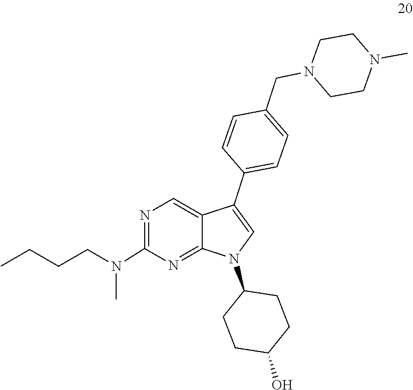 [SMILES: CCCCN(C)c1ncc2c(-c3ccc(CN4CCN(C)CC4)cc3)cn([C@H]3CC[C@H](O)CC3)c2n1]

wherein

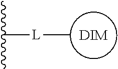

is attached to a modifiable carbon, oxygen, nitrogen or sulfur atom.

In some embodiments, TAMBM is selected from a moiety recited in Huey, M. G. et al., Targeting the TAM Receptors in Leukemia, Cancers (2016) 8(11): 101/1-101/22, such as, for example:

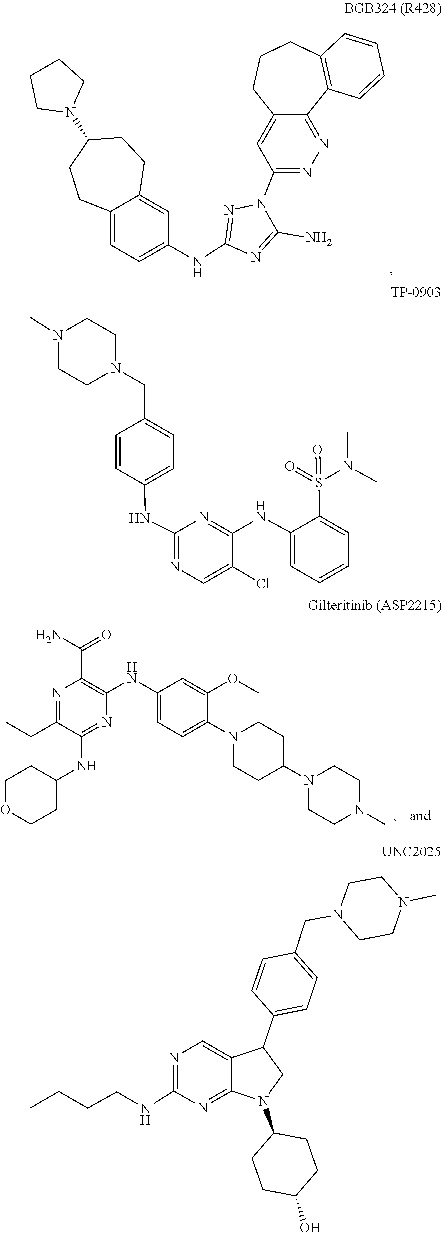

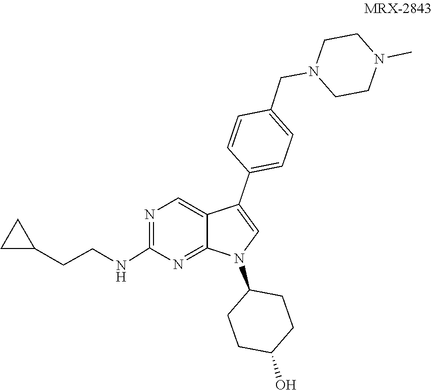 [SMILES: CN1CCN(Cc2ccc(-c3cn([C@H]4CC[C@H](O)CC4)c4nc(NCCC5CC5)ncc34)cc2)CC1]

wherein

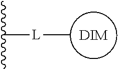

is attached to a modifiable carbon, oxygen, nitrogen or sulfur atom.

In some embodiments, TAMBM is selected from a moiety recited in DeRyckere, A. B. et al., UNC2025, a MERTK Small-Molecule Inhibitor, Is Therapeutically Effective Alone and in Combination with Methotrexate in Leukemia Models, Clin. Cancer Res. (2017) 23(6): 1481-92; Sufit, A. et al., MERTK Inhibition Induces Polyploidy and Promotes Cell Death and Cellular Senescence in Glioblastoma Multiforme, PLoS One (2016) 11(10); e0165107/1-20; Cummings, C. T. et al., Small Molecule Inhibition of MERTK is Efficacious in Non-Small Cell lung Cancer Models Independent of Driver Oncogene Status, Mol. Cancer. Ther. (2015) 14(9): 2014-22; Shi, C. et al., The proto-oncogene Mer tyrosine kinase is a novel therapeutic target in mantle cell lymphoma, J. Hematol. Oncol. (2018) 11(1): 43; and Branchford, B. R. et al., The small-molecule MERTK inhibitor UNC2025 decreases platelet activation and prevents thrombosis, J. Thromb. Haemost. (2018) 16(2): 352-63 such as, for example:

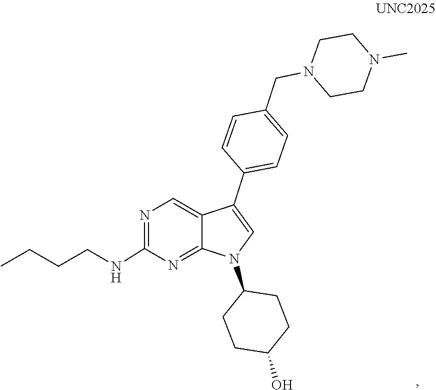 [SMILES: CCCCNc1ncc2c(-c3ccc(CN4CCN(C)CC4)cc3)cn([C@H]3CC[C@H](O)CC3)c2n1]

wherein

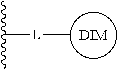

is attached to a modifiable carbon, oxygen, nitrogen or sulfur atom.

In some embodiments, TAMBM is selected from a moiety recited in Paolino, M. et al., The E3 ligase Cbl-b and TAM receptors regulate cancer metastasis via natural killer cells, Nature (2014) 507(7493): 508-12, such as, for example:

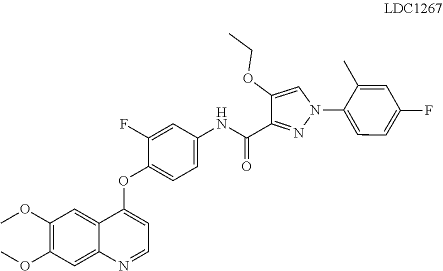 [SMILES: CCOc1cn(-c2ccc(F)cc2C)nc1C(=O)Nc1ccc(Oc2ccnc3cc(OC)c(OC)cc23)c(F)c1]

wherein

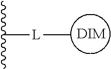

is attached to a modifiable carbon, oxygen, nitrogen or sulfur atom.

In some embodiments, TAMBM is selected from a moiety recited in Cummings, C. T. et al., Molecular Pathways: MERTK Signaling in Cancer, Clin. Cancer Res. (2013) 19(19): 5275-80, such as, for example:

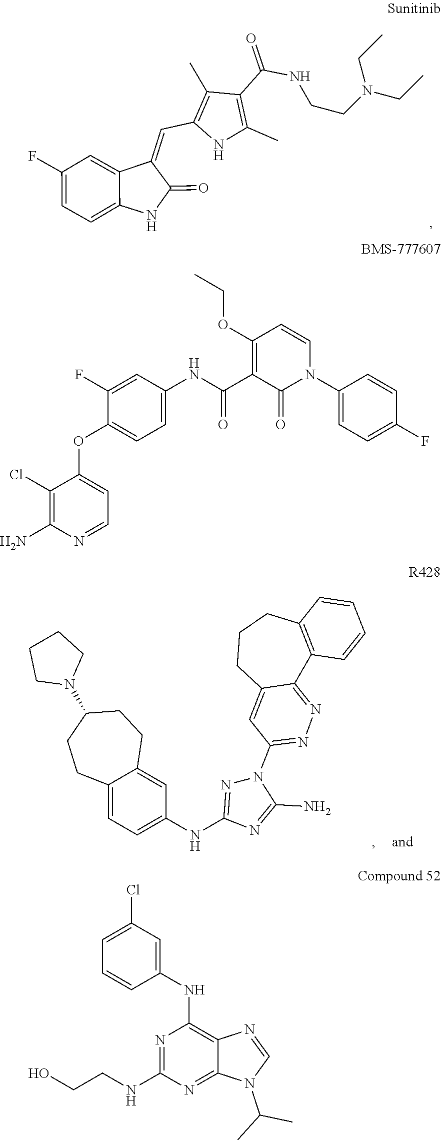 [SMILES: CCN(CC)CCNC(=O)c1c(C)[nH]c(/C=C2\C(=O)Nc3ccc(F)cc32)c1C ; CCOc1ccn(-c2ccc(F)cc2)c(=O)c1C(=O)Nc1ccc(Oc2ccnc(N)c2Cl)c(F)c1 ; Nc1nc(Nc2ccc3c(c2)CC[C@@H](N2CCCC2)CC3)nn1-c1cc2c(nn1)-c1ccccc1CCC2 ; CC(C)n1cnc2c(Nc3cccc(Cl)c3)nc(NCCO)nc21]

wherein

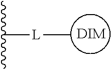

is attached to a modifiable carbon, oxygen, nitrogen or sulfur atom.

In some embodiments, TAMBM is selected from a moiety recited in Schlegel, J. et al., MERTK receptor tyrosine kinase is a therapeutic target in melanoma, J. Clin. Invest. (2013) 123(5): 2257-67; and Ho, Y. J. et al., MerTK is a novel therapeutic target in gastric cancer, Oncotarget (2017) 8(57): 96656-67, such as, for example:

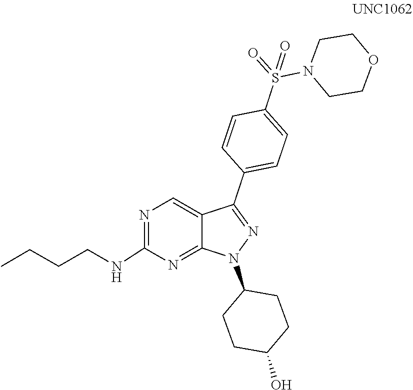 [SMILES: CCCCNc1ncc2c(-c3ccc(S(=O)(=O)N4CCOCC4)cc3)nn([C@H]3CC[C@H](O)CC3)c2n1]

wherein

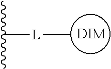

is attached to a modifiable carbon, oxygen, nitrogen or sulfur atom.

In some embodiments, TAMBM is selected from a moiety recited in Espindola, M.S. et al., Targeting TAM Receptors Ameliorates Fibrotic Mechanisms in Idiopathic Pulmonary Fibrosis, Am. J. Resp. Critical Med. (2018) 197(11): 1443-56; and Mikaella, V. & Sassan, H., TAM Receptor Tyrosine Kinases in Cancer Drug Resistance, Cancer Res. (2017) 77(11): 2775-8, such as, for example:

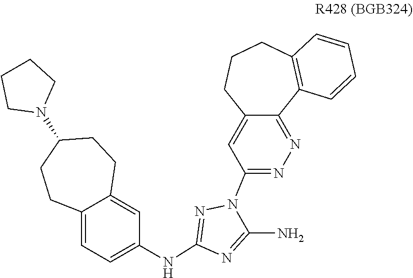 [SMILES: Nc1nc(Nc2ccc3c(c2)CC[C@@H](N2CCCC2)CC3)nn1-c1cc2c(nn1)-c1ccccc1CCC2]

wherein

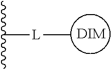

is attached to a modifiable carbon, oxygen, nitrogen or sulfur atom.

In some embodiments, TAMBM is selected from a moiety recited in Kimani, S. G. et al., Small molecule inhibitors block Gas6-inducible TAM activation and tumorigenicity, Scientific Reports (2017) 7: 74908, such as, for example:

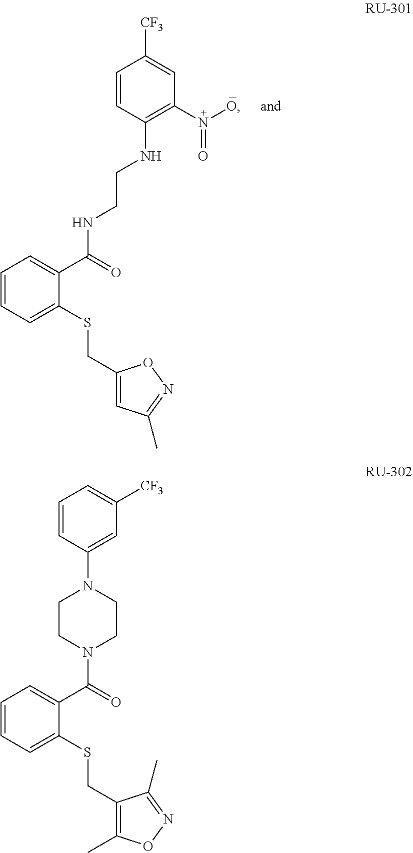

wherein

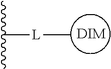

is attached to a modifiable carbon, oxygen, nitrogen or sulfur atom.

In some embodiments, TAMBM is selected from a moiety recited in Schoumacher, M. & Burbridge, M., Key Roles of AXL and MER Receptor Tyrosine Kinases in Resistance to Multiple Anticancer Therapies, Curr. Oncol. Rep. (2017) 19(3):19, such as, for example:

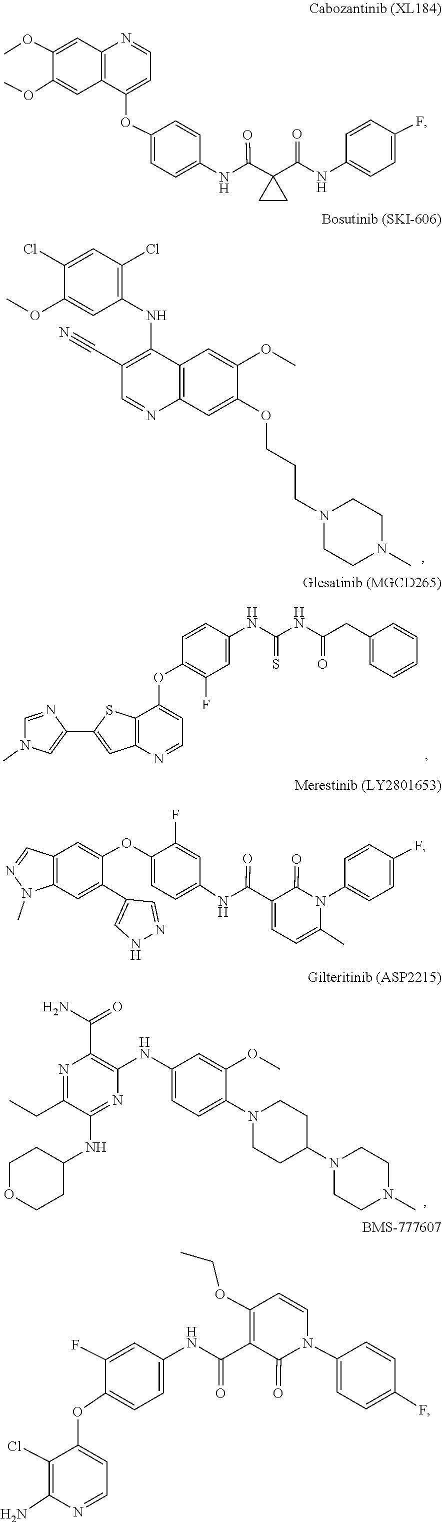

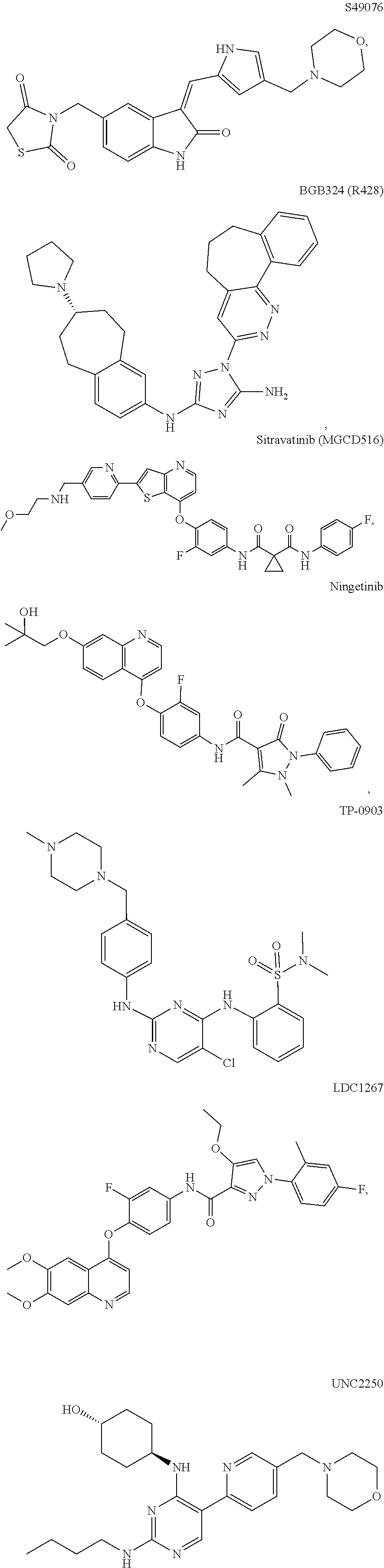

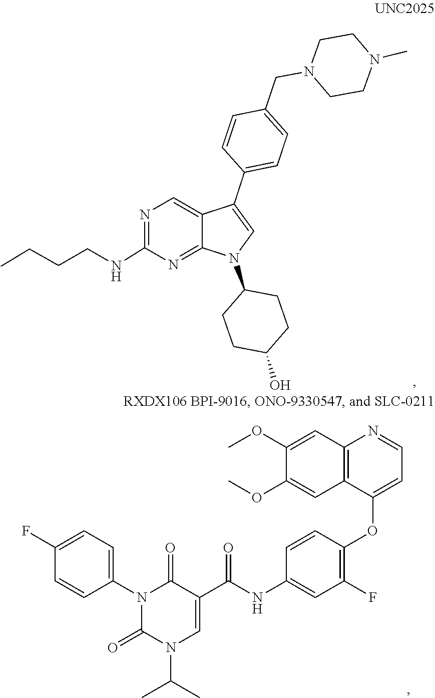 [SMILES: CCCCNc1ncc2c(-c3ccc(CN4CCN(C)CC4)cc3)cn([C@H]3CC[C@H](O)CC3)c2n1 ; COc1cc2nccc(Oc3ccc(NC(=O)c4cn(C(C)C)c(=O)n(-c5ccc(F)cc5)c4=O)cc3F)c2cc1OC]

wherein

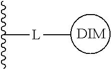

is attached to a modifiable carbon, oxygen, nitrogen or sulfur atom.

In some embodiments, TAMBM is selected from a moiety recited in Davra, V. et al., Ligand Activation of TAM Family Receptors-Implications for Tumor Biology and Therapeutic Response, Cancers (Basel) (2016) 8(12): 107, such as, for example:

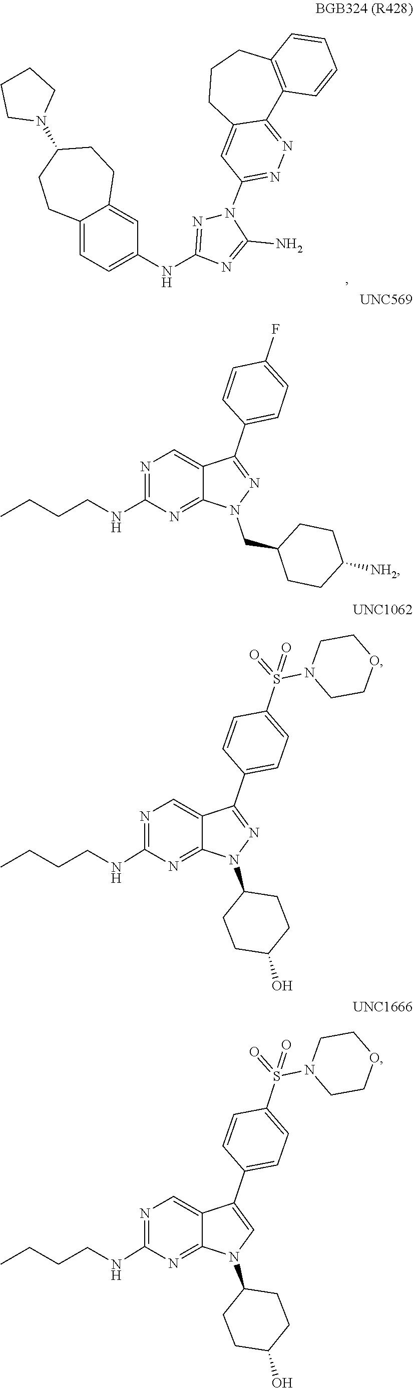 [SMILES: Nc1nc(Nc2ccc3c(c2)CC[C@@H](N2CCCC2)CC3)nn1-c1cc2c(nn1)-c1ccccc1CCC2 ; CCCCNc1ncc2c(-c3ccc(F)cc3)nn(C[C@H]3CC[C@H](N)CC3)c2n1 ; CCCCNc1ncc2c(-c3ccc(S(=O)(=O)N4CCOCC4)cc3)nn([C@H]3CC[C@H](O)CC3)c2n1 ; CCCCNc1ncc2c(-c3ccc(S(=O)(=O)N4CCOCC4)cc3)cn([C@H]3CC[C@H](O)CC3)c2n1]

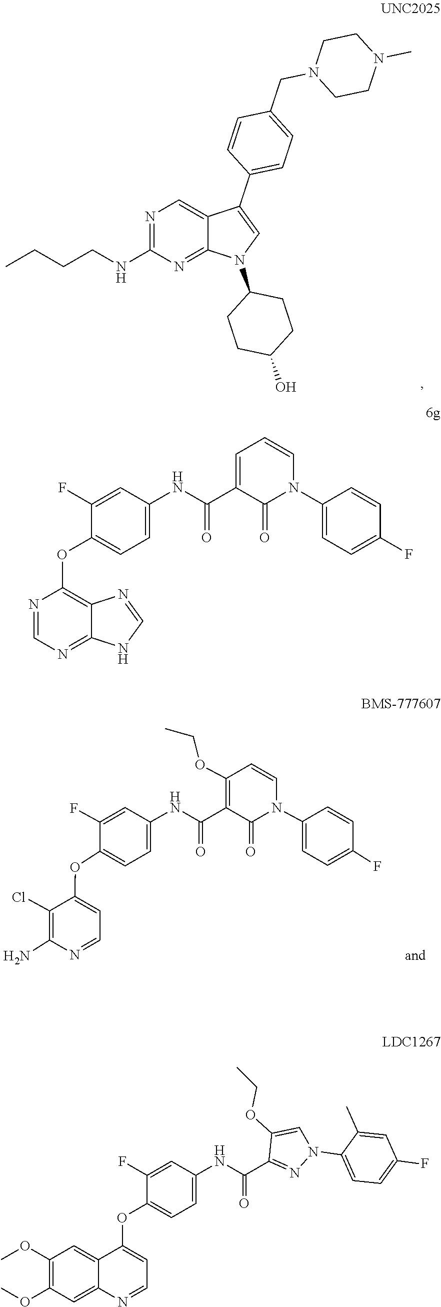

wherein

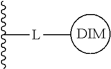

is attached to a modifiable carbon, oxygen, nitrogen or sulfur atom.

In some embodiments, TAMBM is selected from a moiety recited in von Massenhaausen, A. et al., MERTK as a novel therapeutic target in head and neck cancer, Oncotarget (2016) 7(22): 32678-94, such as, for example:

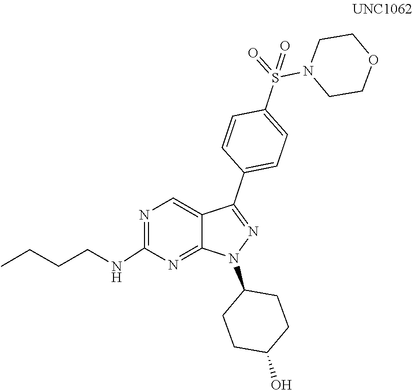 [SMILES: CCCCNc1ncc2c(-c3ccc(S(=O)(=O)N4CCOCC4)cc3)nn([C@H]3CC[C@H](O)CC3)c2n1]

wherein

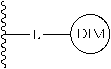

is attached to a modifiable carbon, oxygen, nitrogen or sulfur atom.

In some embodiments, TAMBM is selected from a moiety recited in Lee-Sherick, A. B. et al., Efficacy of a Mer and Flt3 tyrosine kinase small molecule inhibitor, UNC1666, in acute myeloid leukemia, Oncotarget (2015) 6(9): 6722-36, such as, for example:

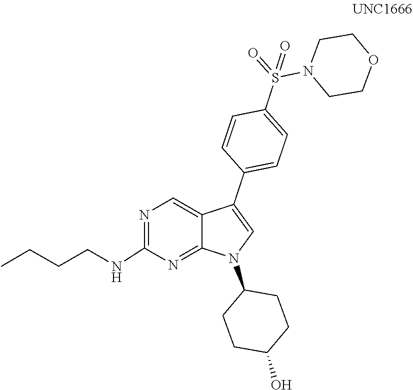 [SMILES: CCCCNc1ncc2c(-c3ccc(S(=O)(=O)N4CCOCC4)cc3)cn([C@H]3CC[C@H](O)CC3)c2n1]

wherein

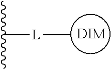

is attached to a modifiable carbon, oxygen, nitrogen or sulfur atom.

In some embodiments, TAMBM is selected from a moiety recited in Christoph, S. et al., UNC569, a novel small-molecule mer inhibitor with efficacy against acute lymphoblastic leukemia in vitro and in vivo, Mol. Cancer. Ther. (2013) 12(11): 2367-77, such as, for example:

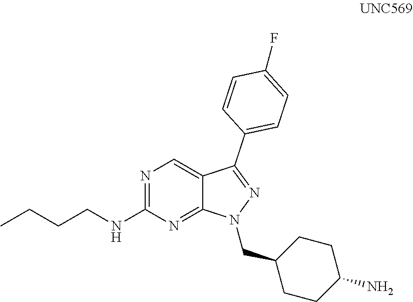 [SMILES: CCCCNc1ncc2c(-c3ccc(F)cc3)nn(C[C@H]3CC[C@H](N)CC3)c2n1]

wherein

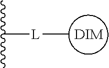

is attached to a modifiable carbon, oxygen, nitrogen or sulfur atom.

In some embodiments, TAMBM is selected from a moiety recited in Liu, J. et al., UNC1062, a new and potent Mer inhibitor, Eur. J. Med. Chem. (2013) 65: 83-93, such as, for example:

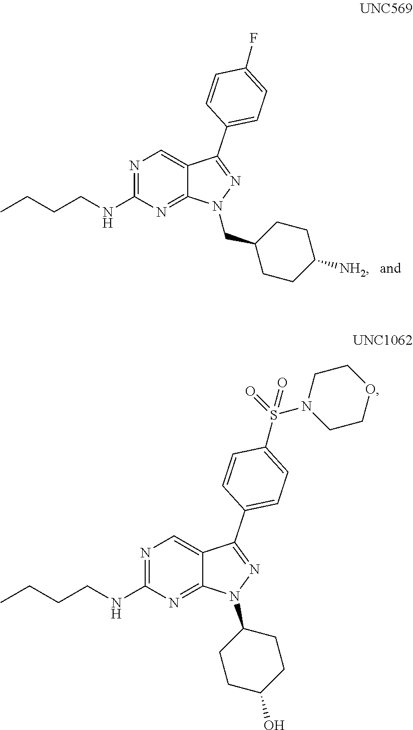 [SMILES: CCCCNc1ncc2c(-c3ccc(F)cc3)nn(C[C@H]3CC[C@H](N)CC3)c2n1 ; "and" ; CCCCNc1ncc2c(-c3ccc(S(=O)(=O)N4CCOCC4)cc3)nn([C@H]3CC[C@H](O)CC3)c2n1]

wherein

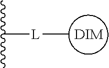

is attached to a modifiable carbon, oxygen, nitrogen or sulfur atom.

In some embodiments, TAMBM is selected from a moiety recited in Suarez, R. M. et al., Inhibitors of the TAM subfamily of tyrosine kinases: Synthesis and biological evaluation, Eur. J. Med. Chem (2013) 61: 2-25, such as, for example:

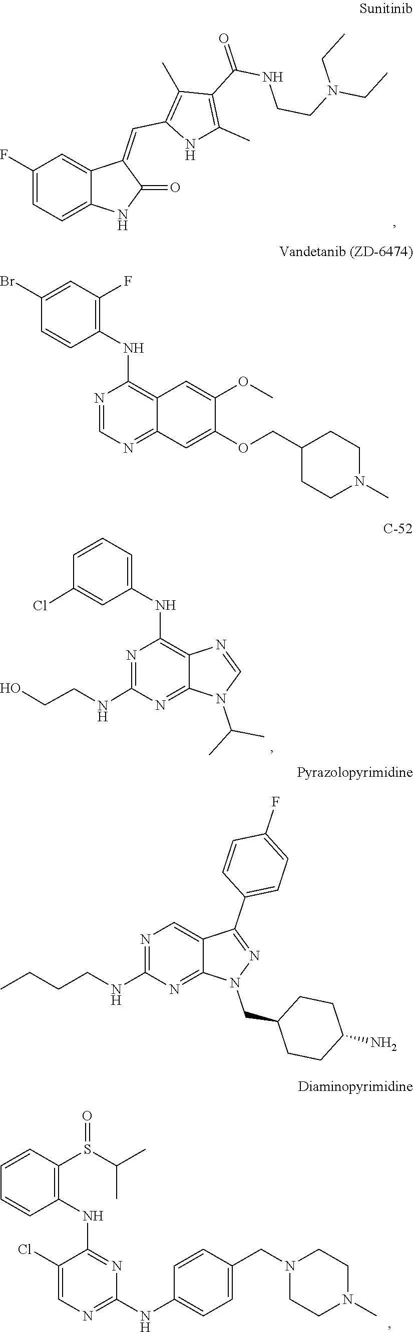

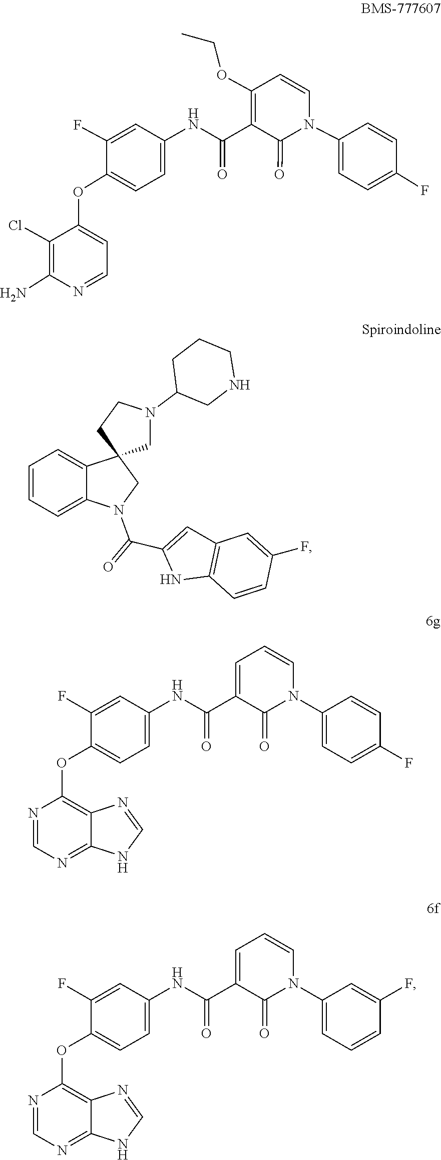 [SMILES: CCOc1ccn(-c2ccc(F)cc2)c(=O)c1C(=O)Nc1ccc(Oc2ccnc(N)c2Cl)c(F)c1 ; O=C(c1cc2cc(F)ccc2[nH]1)N1C[C@]2(CCN(C3CCCNC3)C2)c2ccccc21 ; O=C(Nc1ccc(Oc2ncnc3[nH]cnc23)c(F)c1)c1cccn(-c2ccc(F)cc2)c1=O ; O=C(Nc1ccc(Oc2ncnc3[nH]cnc23)c(F)c1)c1cccn(-c2cccc(F)c2)c1=O]

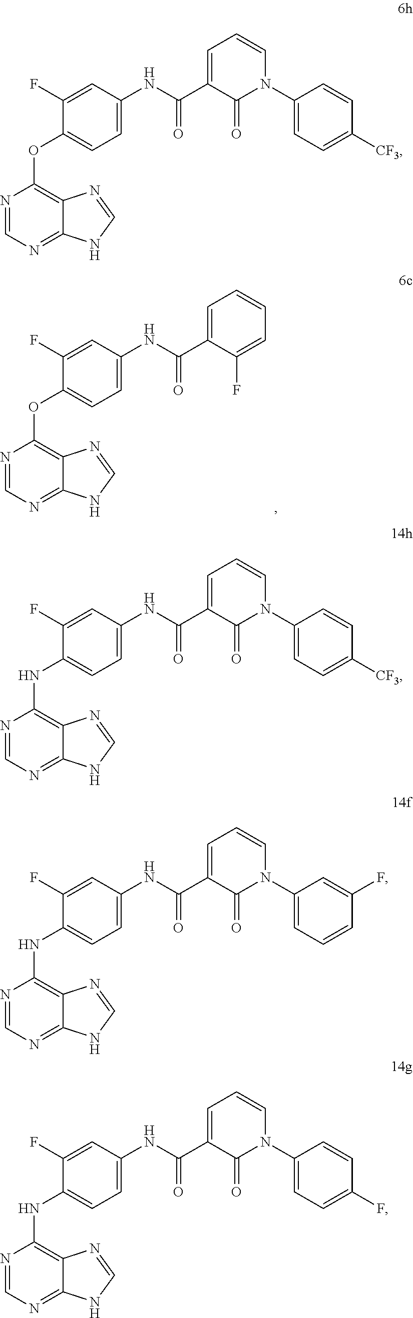 [SMILES: O=C(Nc1ccc(Oc2ncnc3[nH]cnc23)c(F)c1)c1cccn(-c2ccc(C(F)(F)F)cc2)c1=O ; O=C(Nc1ccc(Oc2ncnc3[nH]cnc23)c(F)c1)c1ccccc1F ; O=C(Nc1ccc(Nc2ncnc3[nH]cnc23)c(F)c1)c1cccn(-c2ccc(C(F)(F)F)cc2)c1=O ; O=C(Nc1ccc(Nc2ncnc3[nH]cnc23)c(F)c1)c1cccn(-c2cccc(F)c2)c1=O ; O=C(Nc1ccc(Nc2ncnc3[nH]cnc23)c(F)c1)c1cccn(-c2ccc(F)cc2)c1=O]

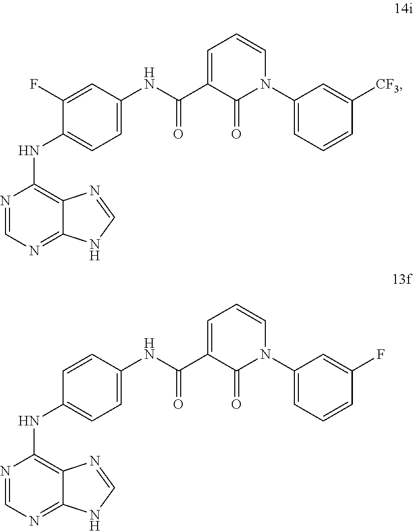 [SMILES: O=C(Nc1ccc(Nc2ncnc3[nH]cnc23)c(F)c1)c1cccn(-c2cccc(C(F)(F)F)c2)c1=O ; O=C(Nc1ccc(Nc2ncnc3[nH]cnc23)cc1)c1cccn(-c2cccc(F)c2)c1=O]

wherein

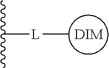

is attached to a modifiable carbon, oxygen, nitrogen or sulfur atom.

In some embodiments, TAMBM is

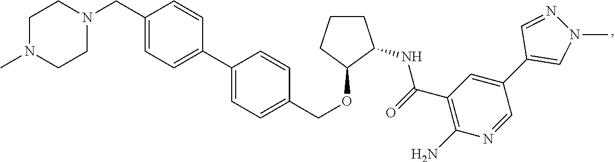

wherein

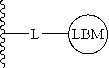

is attached to a modifiable carbon, oxygen, nitrogen or sulfur atom.

In some embodiments, TAMBM is

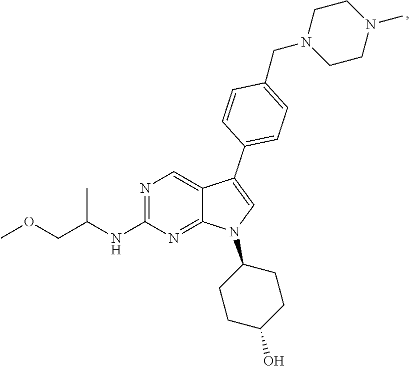

wherein

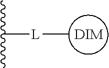

is attached to a modifiable carbon, oxygen, nitrogen or sulfur atom.

In some embodiments, TAMBM is

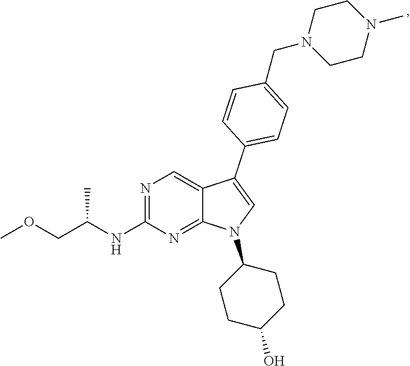

wherein

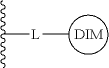

is attached to a modifiable carbon, oxygen, nitrogen or sulfur atom.

In some embodiments, TAMBM is

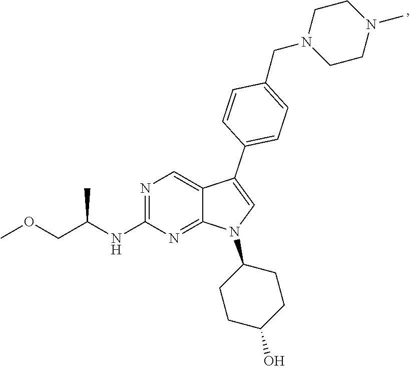

wherein

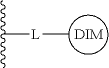

is attached to a modifiable carbon, oxygen, nitrogen or sulfur atom.

In some embodiments, TAMBM is

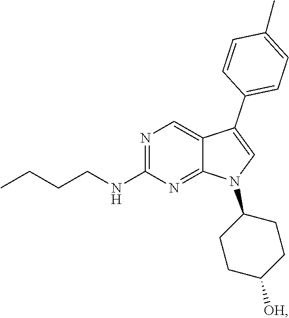 [SMILES: CCCCNc1ncc2c(-c3ccc(C)cc3)cn([C@H]3CC[C@H](O)CC3)c2n1]

wherein

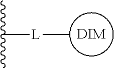

is attached to a modifiable carbon, oxygen, nitrogen or sulfur atom.

In some embodiments, TAMBM is

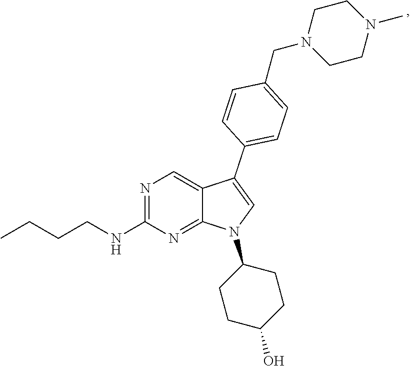 [SMILES: CCCCNc1ncc2c(-c3ccc(CN4CCN(C)CC4)cc3)cn([C@H]3CC[C@H](O)CC3)c2n1]

wherein

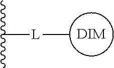

is attached to a modifiable carbon, oxygen, nitrogen or sulfur atom.

In some embodiments, TAMBM is

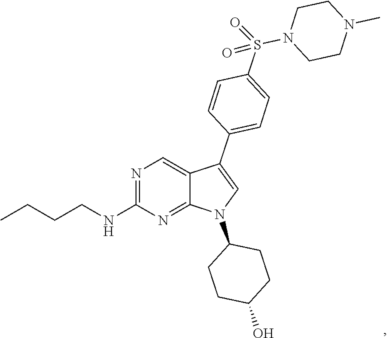 [SMILES: CCCCNc1ncc2c(-c3ccc(S(=O)(=O)N4CCN(C)CC4)cc3)cn([C@H]3CC[C@H](O)CC3)c2n1]

wherein

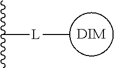

is attached to a modifiable carbon, oxygen, nitrogen or sulfur atom.

In some embodiments, TAMBM is

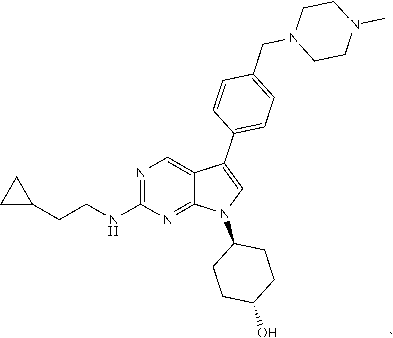 [SMILES: CN1CCN(Cc2ccc(-c3cn([C@H]4CC[C@H](O)CC4)c4nc(NCCC5CC5)ncc34)cc2)CC1]

wherein

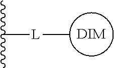

is attached to a modifiable carbon, oxygen, nitrogen or sulfur atom.

In some embodiments, TAMBM is

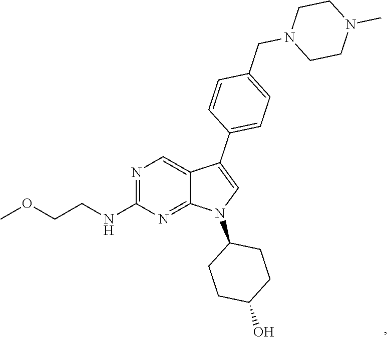

wherein

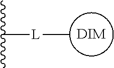

is attached to a modifiable carbon, oxygen, nitrogen or sulfur atom.

In some embodiments, TAMBM is

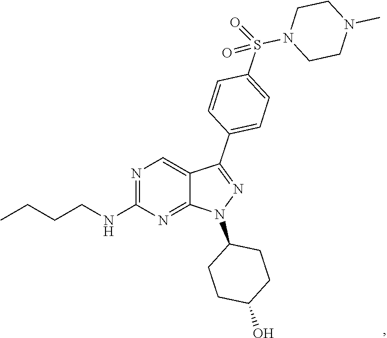 [SMILES: CCCCNc1ncc2c(-c3ccc(S(=O)(=O)N4CCN(C)CC4)cc3)nn([C@H]3CC[C@H](O)CC3)c2n1]

wherein

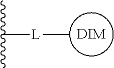

is attached to a modifiable carbon, oxygen, nitrogen or sulfur atom.

In some embodiments, TAMBM is

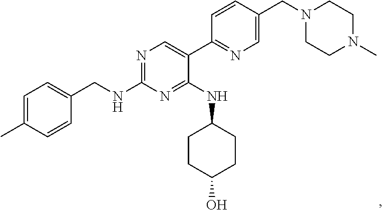

wherein

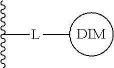

is attached to a modifiable carbon, oxygen, nitrogen or sulfur atom.

In some embodiments, TAMBM is

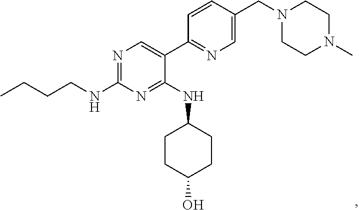 [SMILES: CCCCNc1ncc(-c2ccc(CN3CCN(C)CC3)cn2)c(N[C@H]2CC[C@H](O)CC2)n1]

wherein

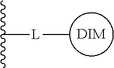

is attached to a modifiable carbon, oxygen, nitrogen or sulfur atom.

In some embodiments, TAMBM is

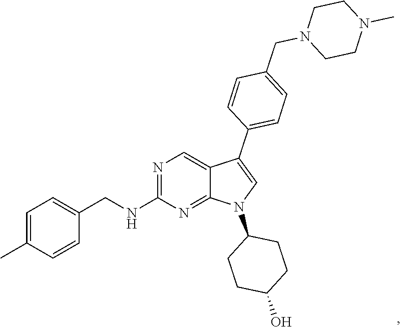

wherein

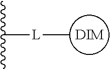

is attached to a modifiable carbon, oxygen, nitrogen or sulfur atom.

In some embodiments, TAMBM is

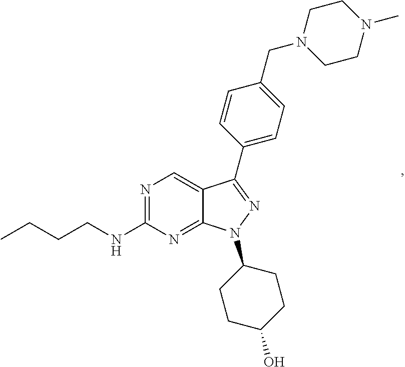 [SMILES: CCCCNc1ncc2c(-c3ccc(CN4CCN(C)CC4)cc3)nn([C@H]3CC[C@H](O)CC3)c2n1]

wherein

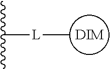

is attached to a modifiable carbon, oxygen, nitrogen or sulfur atom.

In some embodiments, the present invention provides a compound of Formula VII:

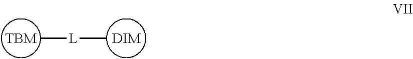

or a pharmaceutically acceptable salt thereof, wherein L and DIM are as described above and herein, and TBM is a TAMBM that preferentially binds Tyro3 (i.e., a Tyro3 binding moiety).

In some embodiments, the present invention provides a compound of Formula VIII:

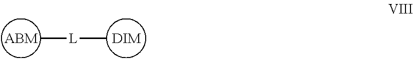

or a pharmaceutically acceptable salt thereof, wherein L and DIM are as described above and herein, and ABM is a TAMBM that preferentially binds Axl (i.e., an Axl binding moiety).

In some embodiments, the present invention provides a compound of Formula V:

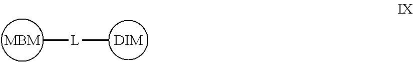

or a pharmaceutically acceptable salt thereof, wherein L and DIM are as described above and herein, and MBM is a TAMBM that preferentially binds MerTK (i.e., a MerTK binding moiety).

In some embodiments, a provided compound or pharmaceutically acceptatable salt thereof, is selected from those wherein TAMBM is

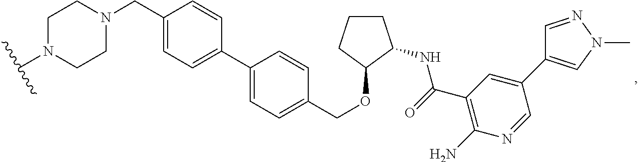 [SMILES: *N1CCN(Cc2ccc(-c3ccc(CO[C@H]4CCC[C@@H]4NC(=O)c4cc(-c5cnn(C)c5)cnc4N)cc3)cc2)CC1]

LBM is selected from any of those in Table A below, and L is selected from any of those in Table B below.

In some embodiments, a provided compound or pharmaceutically acceptatable salt thereof, is selected from those wherein TAMBM is

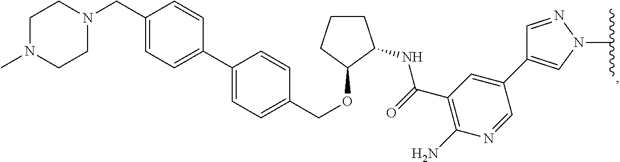 [SMILES: *n1cc(-c2cnc(N)c(C(=O)N[C@H]3CCC[C@@H]3OCc3ccc(-c4ccc(CN5CCN(C)CC5)cc4)cc3)c2)cn1]

LBM is selected from any of those in Table A below, and L is selected from any of those in Table B below.

In some embodiments, a provided compound or pharmaceutically acceptatable salt thereof, is selected from those wherein TAMBM is

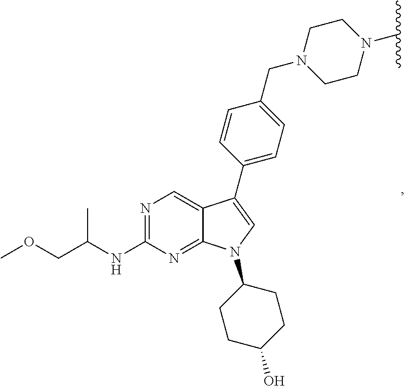 [SMILES: *N1CCN(Cc2ccc(-c3cn([C@H]4CC[C@H](O)CC4)c4nc(NC(C)COC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

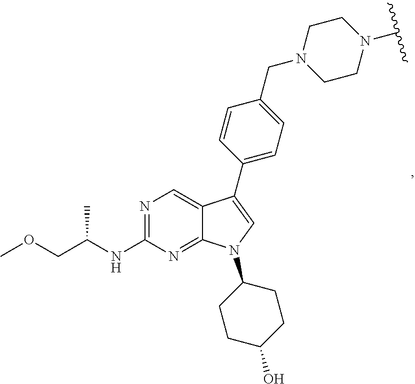 [SMILES: *N1CCN(Cc2ccc(-c3cn([C@H]4CC[C@H](O)CC4)c4nc(N[C@@H](C)COC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

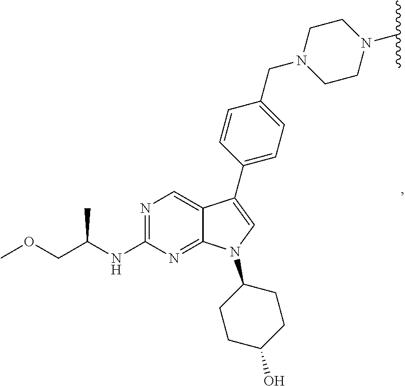 [SMILES: *N1CCN(Cc2ccc(-c3cn([C@H]4CC[C@H](O)CC4)c4nc(N[C@H](C)COC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

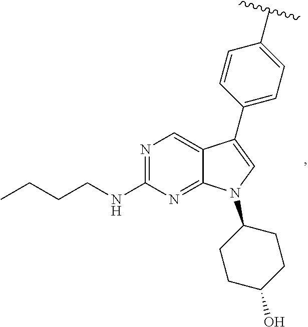 [SMILES: *c1ccc(-c2cn([C@H]3CC[C@H](O)CC3)c3nc(NCCCC)ncc23)cc1]

LBM is selected from any of those in Table A below, and L is selected from any of those in Table B below.

In some embodiments, a provided compound or pharmaceutically acceptatable salt thereof, is selected from those wherein TAMBM is

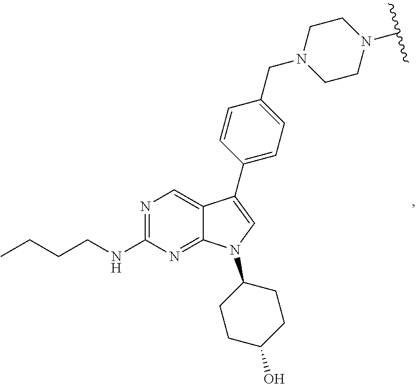 [SMILES: *N1CCN(Cc2ccc(-c3cn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

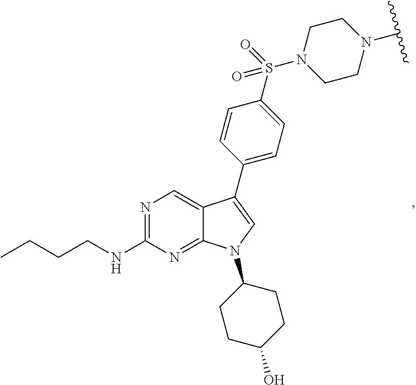 [SMILES: *N1CCN(S(=O)(=O)c2ccc(-c3cn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

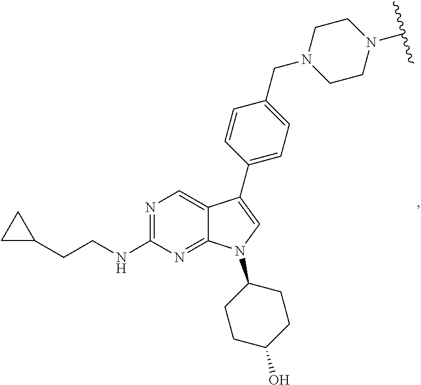 [SMILES: *N1CCN(Cc2ccc(-c3cn([C@H]4CC[C@H](O)CC4)c4nc(NCCC5CC5)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

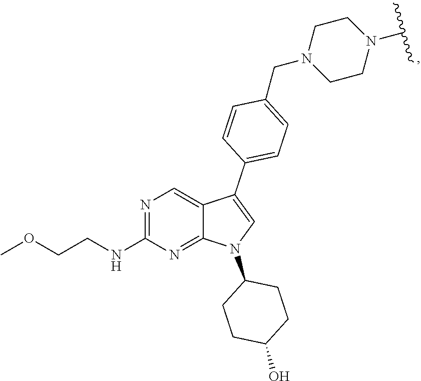 [SMILES: *N1CCN(Cc2ccc(-c3cn([C@H]4CC[C@H](O)CC4)c4nc(NCCOC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

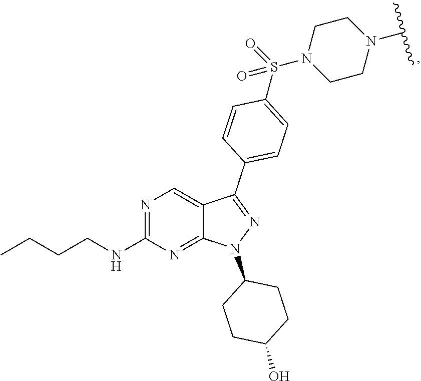 [SMILES: *N1CCN(S(=O)(=O)c2ccc(-c3nn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

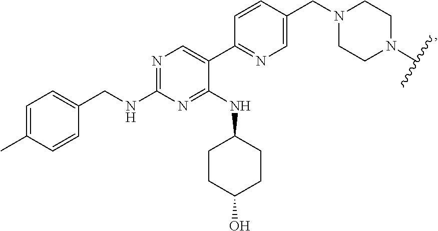 [SMILES: *N1CCN(Cc2ccc(-c3cnc(NCc4ccc(C)cc4)nc3N[C@H]3CC[C@H](O)CC3)nc2)CC1]

LBM is selected from any of those in Table A below, and L is selected from any of those in Table B below.

In some embodiments, a provided compound or pharmaceutically acceptatable salt thereof, is selected from those wherein TAMBM is

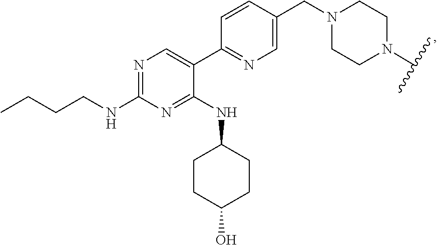 [SMILES: *N1CCN(Cc2ccc(-c3cnc(NCCCC)nc3N[C@H]3CC[C@H](O)CC3)nc2)CC1]

LBM is selected from any of those in Table A below, and L is selected from any of those in Table B below.

In some embodiments, a provided compound or pharmaceutically acceptatable salt thereof, is selected from those wherein TAMBM is

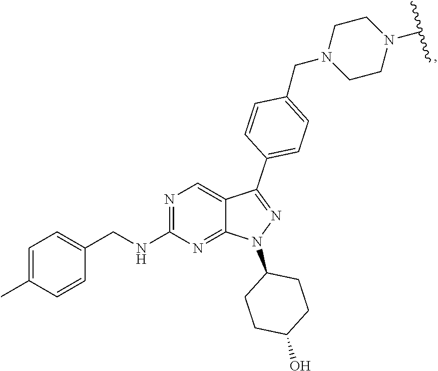 [SMILES: *N1CCN(Cc2ccc(-c3nn([C@H]4CC[C@H](O)CC4)c4nc(NCc5ccc(C)cc5)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

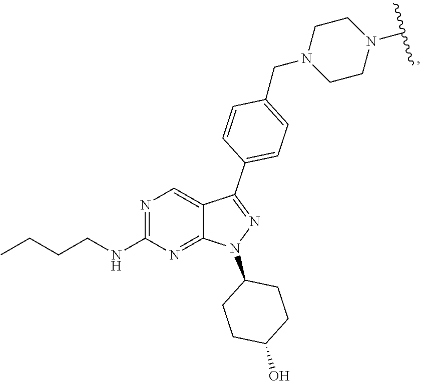 [SMILES: *N1CCN(Cc2ccc(-c3nn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

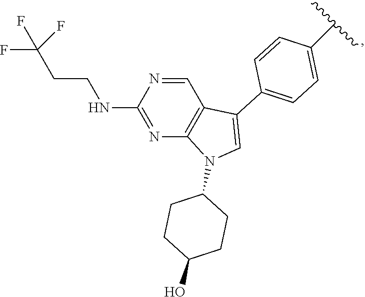 [SMILES: *c1ccc(-c2cn([C@H]3CC[C@H](O)CC3)c3nc(NCCC(F)(F)F)ncc23)cc1]

LBM is selected from any of those in Table A below, and L is selected from any of those in Table B below.

In some embodiments, a provided compound or pharmaceutically acceptatable salt thereof, is selected from those wherein TAMBM is

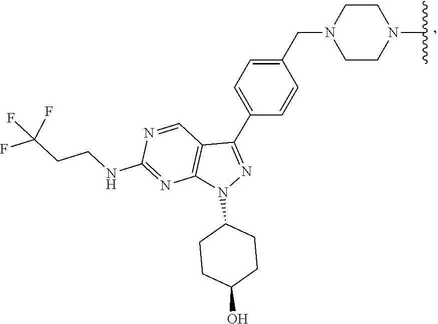 [SMILES: *N1CCN(Cc2ccc(-c3nn([C@H]4CC[C@H](O)CC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

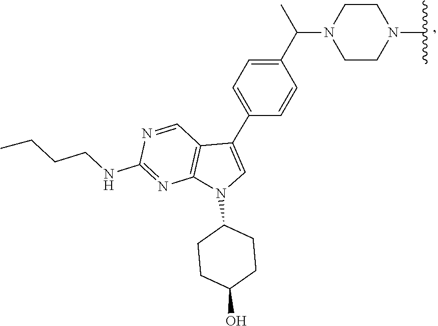 [SMILES: *N1CCN(C(C)c2ccc(-c3cn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

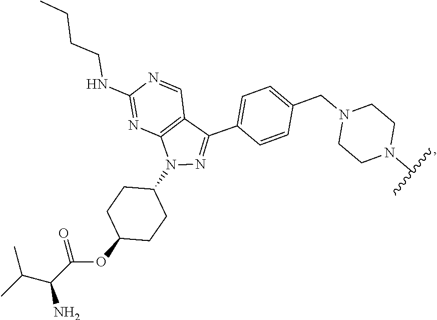 [SMILES: *N1CCN(Cc2ccc(-c3nn([C@H]4CC[C@H](OC(=O)[C@@H](N)C(C)C)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

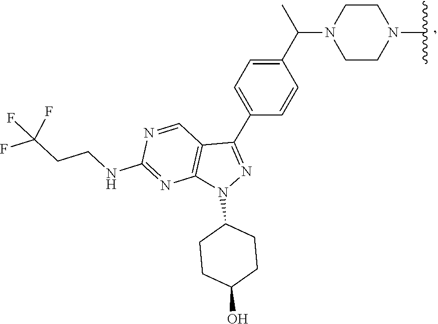 [SMILES: *N1CCN(C(C)c2ccc(-c3nn([C@H]4CC[C@H](O)CC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

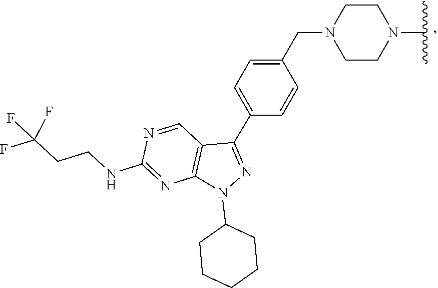 [SMILES: *N1CCN(Cc2ccc(-c3nn(C4CCCCC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

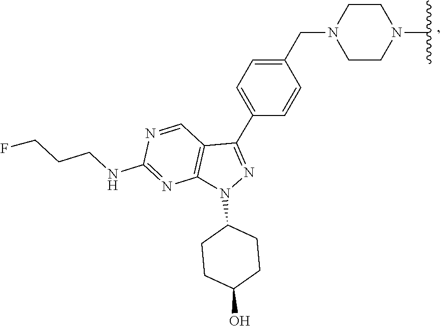 [SMILES: *N1CCN(Cc2ccc(-c3nn([C@H]4CC[C@H](O)CC4)c4nc(NCCCF)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

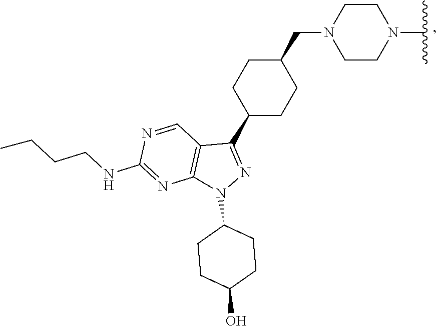 [SMILES: *N1CCN(C[C@H]2CC[C@@H](c3nn([C@H]4CC[C@H](O)CC4)c4nc(NCCCC)ncc43)CC2)CC1]

LBM is selected from any of those in Table A below, and L is selected from any of those in Table B below.

In some embodiments, a provided compound or pharmaceutically acceptatable salt thereof, is selected from those wherein TAMBM is

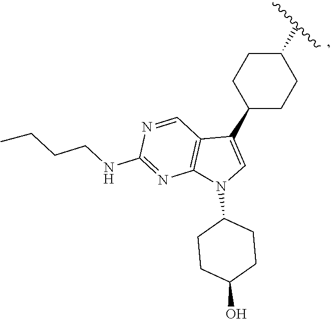 [SMILES: *[C@H]1CC[C@H](c2cn([C@H]3CC[C@H](O)CC3)c3nc(NCCCC)ncc32)CC1]

LBM is selected from any of those in Table A below, and L is selected from any of those in Table B below.

In some embodiments, a provided compound or pharmaceutically acceptatable salt thereof, is selected from those wherein TAMBM is

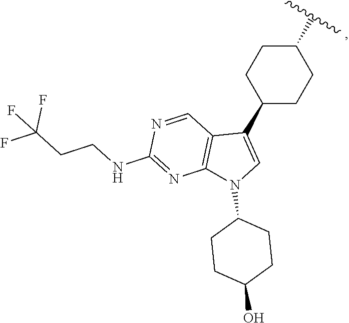 [SMILES: *[C@H]1CC[C@H](c2cn([C@H]3CC[C@H](O)CC3)c3nc(NCCC(F)(F)F)ncc32)CC1]

LBM is selected from any of those in Table A below, and L is selected from any of those in Table B below.

In some embodiments, a provided compound or pharmaceutically acceptatable salt thereof, is selected from those wherein TAMBM is

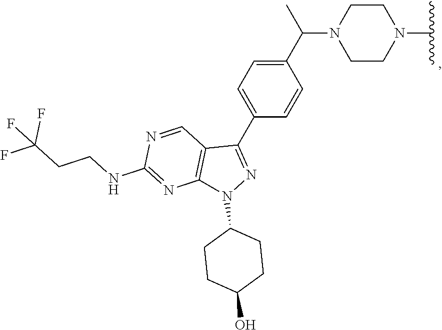 [SMILES: *N1CCN(C(C)c2ccc(-c3nn([C@H]4CC[C@H](O)CC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

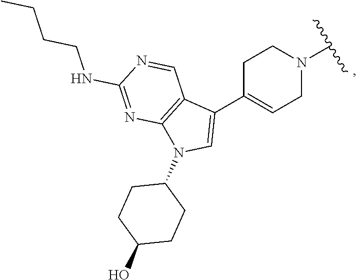

LBM is selected from any of those in Table A below, and L is selected from any of those in Table B below.

In some embodiments, a provided compound or pharmaceutically acceptatable salt thereof, is selected from those wherein TAMBM is

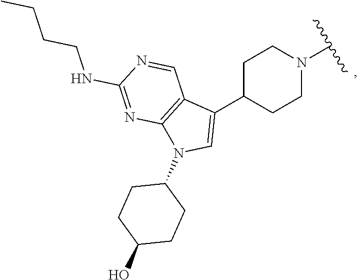

LBM is selected from any of those in Table A below, and L is selected from any of those in Table B below.

In some embodiments, a provided compound or pharmaceutically acceptatable salt thereof, is selected from those wherein TAMBM is

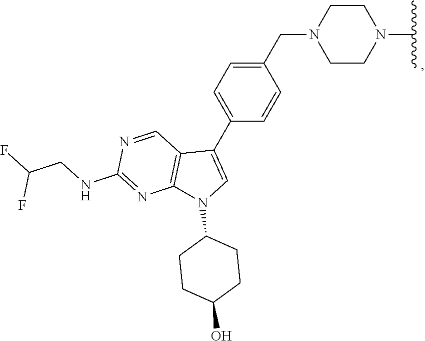 [SMILES: *N1CCN(Cc2ccc(-c3cn([C@H]4CC[C@H](O)CC4)c4nc(NCC(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

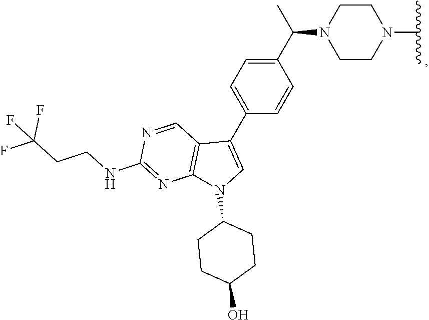 [SMILES: *N1CCN([C@H](C)c2ccc(-c3cn([C@H]4CC[C@H](O)CC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

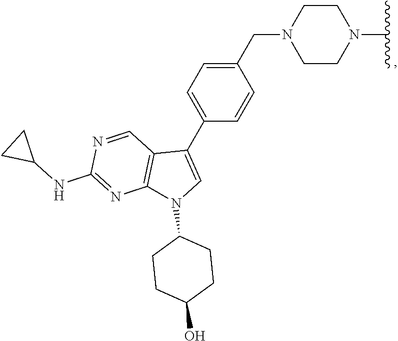 [SMILES: *N1CCN(Cc2ccc(-c3cn([C@H]4CC[C@H](O)CC4)c4nc(NC5CC5)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

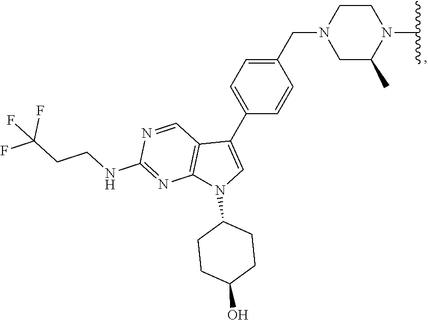 [SMILES: *N1CCN(Cc2ccc(-c3cn([C@H]4CC[C@H](O)CC4)c4nc(NCCC(F)(F)F)ncc34)cc2)C[C@@H]1C]

LBM is selected from any of those in Table A below, and L is selected from any of those in Table B below.

In some embodiments, a provided compound or pharmaceutically acceptatable salt thereof, is selected from those wherein TAMBM is

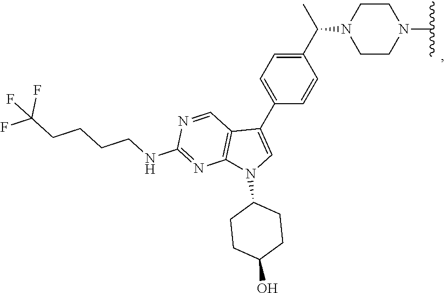 [SMILES: *N1CCN([C@@H](C)c2ccc(-c3cn([C@H]4CC[C@H](O)CC4)c4nc(NCCCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is LBM

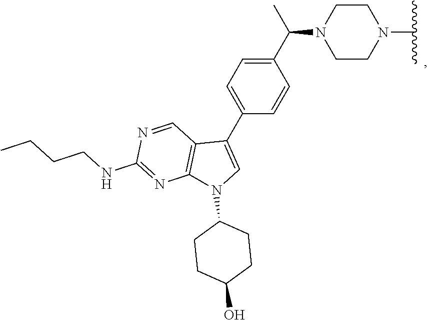 [SMILES: *N1CCN([C@H](C)c2ccc(-c3cn([C@H]4CC[C@H](O)CC4)c4nc(NCCCC)ncc34)cc2)CC1]

is selected from any of those in Table A below, and L is selected from any of those in Table B below.

In some embodiments, a provided compound or pharmaceutically acceptatable salt thereof, is selected from those wherein TAMBM is

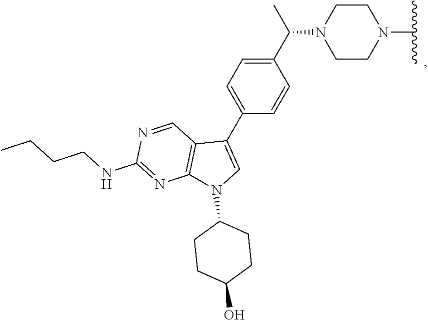 [SMILES: *N1CCN([C@@H](C)c2ccc(-c3cn([C@H]4CC[C@H](O)CC4)c4nc(NCCCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

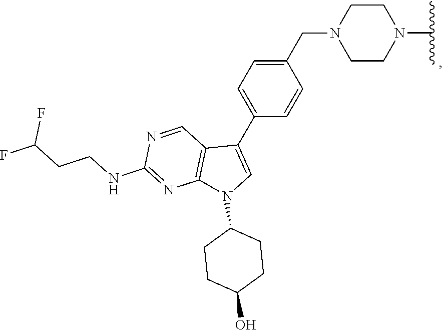 [SMILES: *N1CCN(Cc2ccc(-c3cn([C@H]4CC[C@H](O)CC4)c4nc(NCCC(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

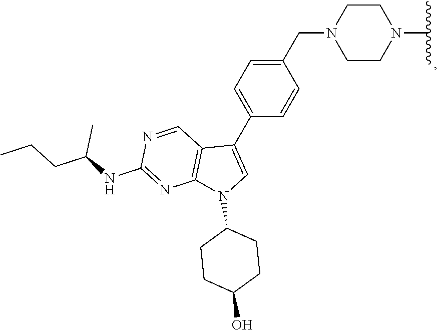 [SMILES: *N1CCN(Cc2ccc(-c3cn([C@H]4CC[C@H](O)CC4)c4nc(N[C@H](C)CCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

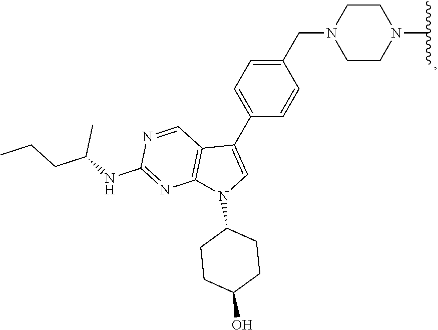 [SMILES: *N1CCN(Cc2ccc(-c3cn([C@H]4CC[C@H](O)CC4)c4nc(N[C@@H](C)CCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

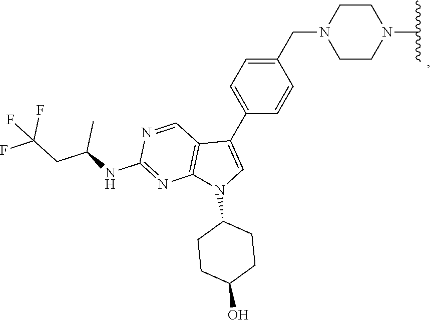 [SMILES: *N1CCN(Cc2ccc(-c3cn([C@H]4CC[C@H](O)CC4)c4nc(N[C@H](C)CC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

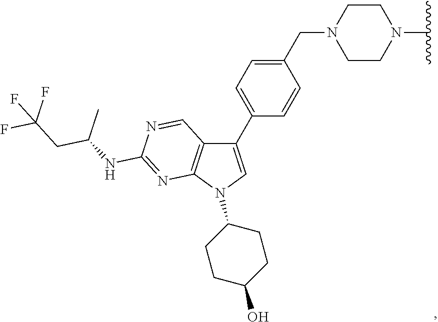 [SMILES: *N1CCN(Cc2ccc(-c3cn([C@H]4CC[C@H](O)CC4)c4nc(N[C@@H](C)CC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

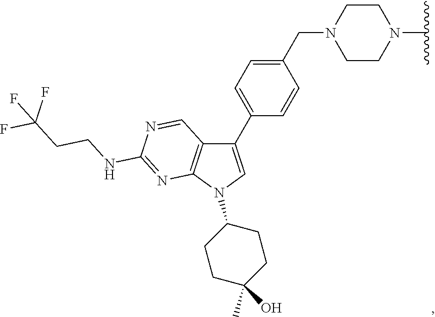 [SMILES: *N1CCN(Cc2ccc(-c3cn([C@H]4CC[C@](C)(O)CC4)c4nc(NCCC(F)(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

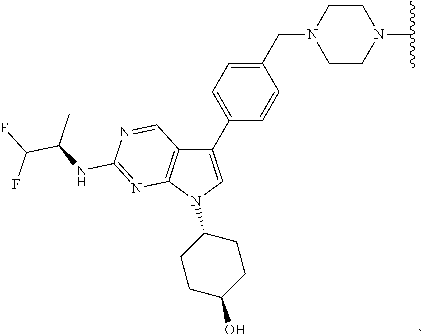 [SMILES: *N1CCN(Cc2ccc(-c3cn([C@H]4CC[C@H](O)CC4)c4nc(N[C@H](C)C(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

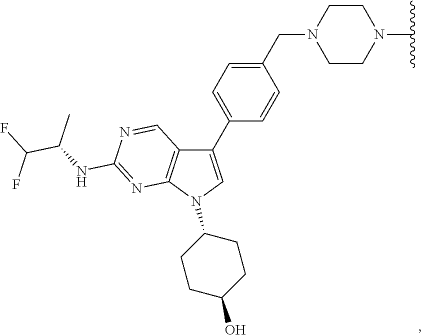 [SMILES: *N1CCN(Cc2ccc(-c3cn([C@H]4CC[C@H](O)CC4)c4nc(N[C@@H](C)C(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

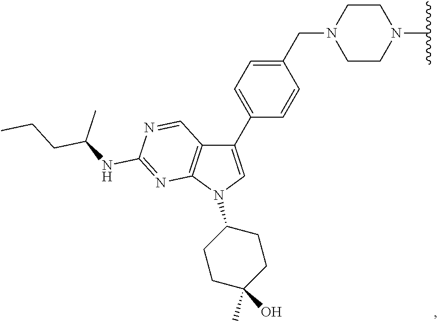 [SMILES: *N1CCN(Cc2ccc(-c3cn([C@H]4CC[C@](C)(O)CC4)c4nc(N[C@H](C)CCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

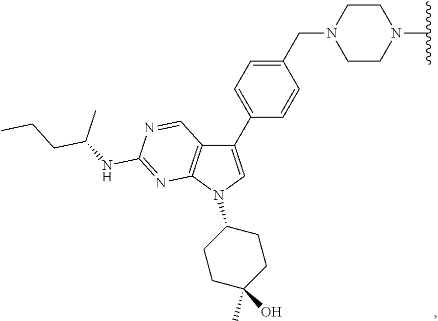 [SMILES: *N1CCN(Cc2ccc(-c3cn([C@H]4CC[C@](C)(O)CC4)c4nc(N[C@@H](C)CCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

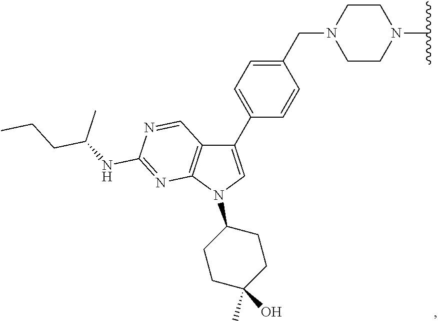 [SMILES: *N1CCN(Cc2ccc(-c3cn([C@H]4CC[C@@](C)(O)CC4)c4nc(N[C@@H](C)CCC)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

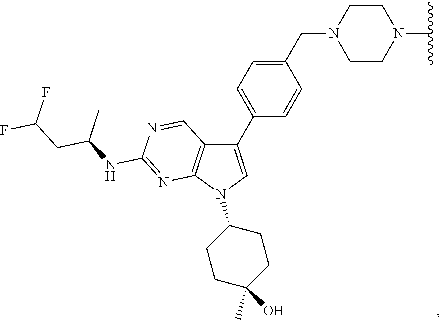 [SMILES: *N1CCN(Cc2ccc(-c3cn([C@H]4CC[C@](C)(O)CC4)c4nc(N[C@H](C)CC(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

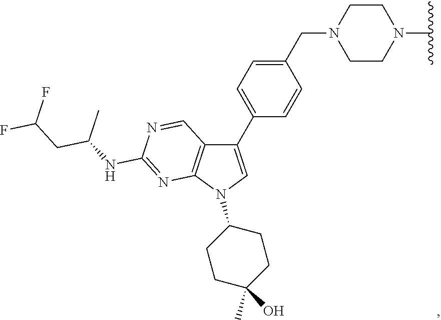 [SMILES: *N1CCN(Cc2ccc(-c3cn([C@H]4CC[C@](C)(O)CC4)c4nc(N[C@@H](C)CC(F)F)ncc34)cc2)CC1]

LBM is selected from any of those in Table A below, and L is selected from any of those in Table B below.

In some embodiments, a provided compound or pharmaceutically acceptatable salt thereof, is selected from those wherein TAMBM is

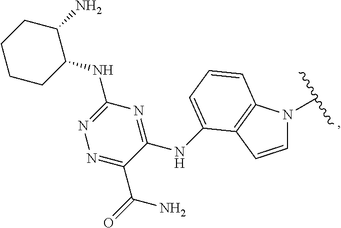 [SMILES: *n1ccc2c(Nc3nc(N[C@@H]4CCCC[C@@H]4N)nnc3C(N)=O)cccc21]

LBM is selected from any of those in Table A below, and L is selected from any of those in Table B below.

In some embodiments, a provided compound or pharmaceutically acceptatable salt thereof, is selected from those wherein TAMBM is

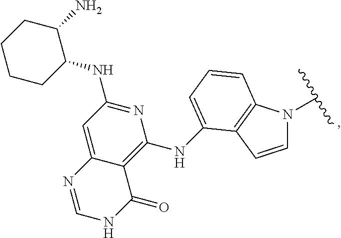 [SMILES: *n1ccc2c(Nc3nc(N[C@@H]4CCCC[C@@H]4N)cc4nc[nH]c(=O)c34)cccc21]

LBM is selected from any of those in Table A below, and L is selected from any of those in Table B below.

In some embodiments, a provided compound or pharmaceutically acceptatable salt thereof, is selected from those wherein TAMBM is

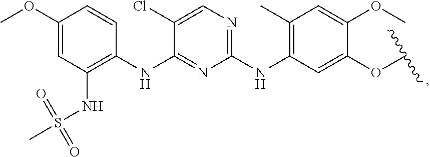 [SMILES: *Oc1cc(Nc2ncc(Cl)c(Nc3ccc(OC)cc3NS(C)(=O)=O)n2)c(C)cc1OC]

LBM is selected from any of those in Table A below, and L is selected from any of those in Table B below.

In some embodiments, a provided compound or pharmaceutically acceptatable salt thereof, is selected from those wherein TAMBM is

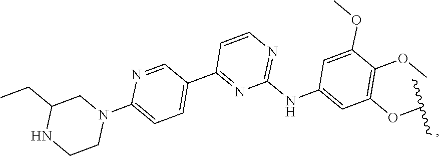 [SMILES: *Oc1cc(Nc2nccc(-c3ccc(N4CCNC(CC)C4)nc3)n2)cc(OC)c1OC]

LBM is selected from any of those in Table A below, and L is selected from any of those in Table B below.

TABLE A
Exemplified E3 ligases (LBM)

| | |
|---|---|
| 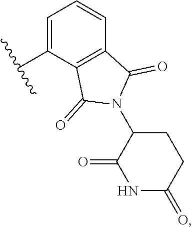 [SMILES: *c1cccc2c1C(=O)N(C1CCC(=O)NC1=O)C2=O] | (a) |
| 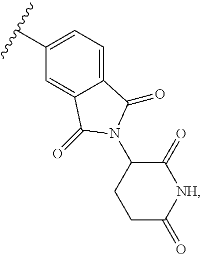 [SMILES: *c1ccc2c(c1)C(=O)N(C1CCC(=O)NC1=O)C2=O] | (b) |
| 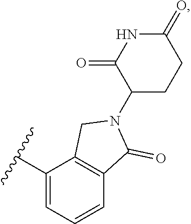 [SMILES: *c1cccc2c1CN(C1CCC(=O)NC1=O)C2=O] | (c) |
| 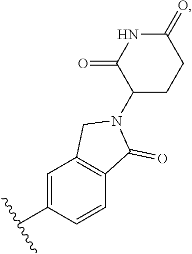 [SMILES: *c1ccc2c(c1)CN(C1CCC(=O)NC1=O)C2=O] | (d) |
| 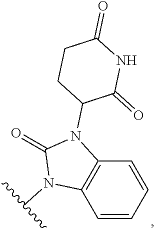 [SMILES: *n1c(=O)n(C2CCC(=O)NC2=O)c2ccccc21] | (e) |
| 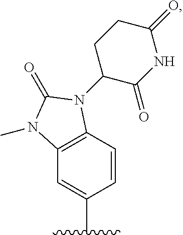 [SMILES: *c1ccc2c(c1)n(C)c(=O)n2C1CCC(=O)NC1=O] | (f) |
| 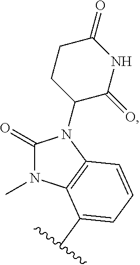 [SMILES: *c1cccc2c1n(C)c(=O)n2C1CCC(=O)NC1=O] | (g) |
| 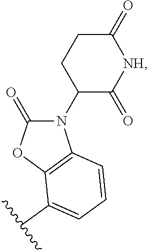 [SMILES: *c1cccc2c1oc(=O)n2C1CCC(=O)NC1=O] | (h) |
| 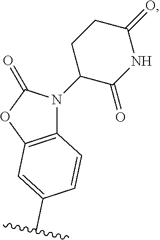 [SMILES: *c1ccc2c(c1)oc(=O)n2C1CCC(=O)NC1=O] | (i) |
| 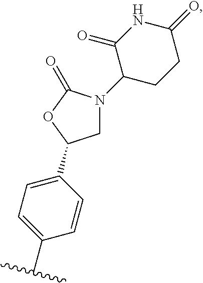 [SMILES: *c1ccc([C@H]2CN(C3CCC(=O)NC3=O)C(=O)O2)cc1] | (j) |
| 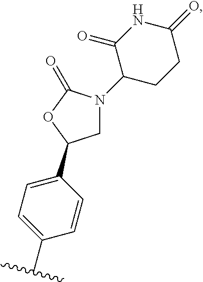 [SMILES: *c1ccc([C@@H]2CN(C3CCC(=O)NC3=O)C(=O)O2)cc1] | (k) |
| 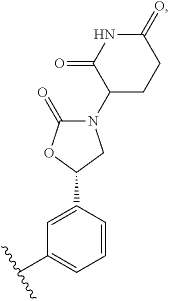 [SMILES: *c1cccc([C@H]2CN(C3CCC(=O)NC3=O)C(=O)O2)c1] | (l) |
| 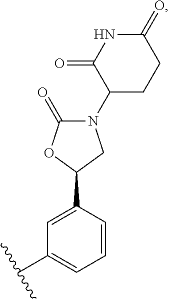 [SMILES: *c1cccc([C@@H]2CN(C3CCC(=O)NC3=O)C(=O)O2)c1] | (m) |
| 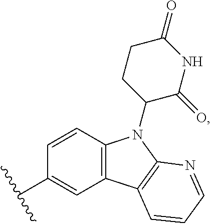 [SMILES: *c1ccc2c(c1)c1cccnc1n2C1CCC(=O)NC1=O] | (n) |
| 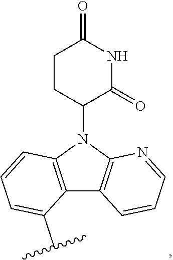 [SMILES: *c1cccc2c1c1cccnc1n2C1CCC(=O)NC1=O] | (o) |
| 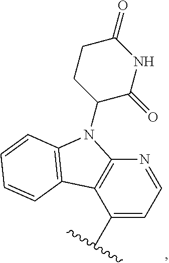 [SMILES: *c1ccnc2c1c1ccccc1n2C1CCC(=O)NC1=O] | (p) |
| 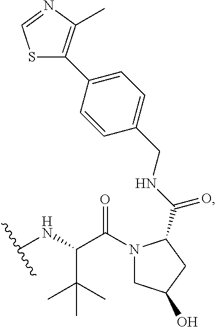 [SMILES: *N[C@H](C(=O)N1C[C@H](O)C[C@H]1C(=O)NCc1ccc(-c2scnc2C)cc1)C(C)(C)C] | (q) |
| 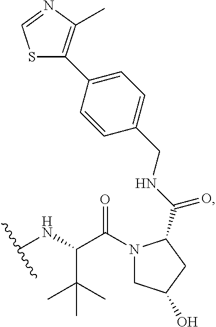 [SMILES: *N[C@H](C(=O)N1C[C@@H](O)C[C@H]1C(=O)NCc1ccc(-c2scnc2C)cc1)C(C)(C)C] | (r) |
| 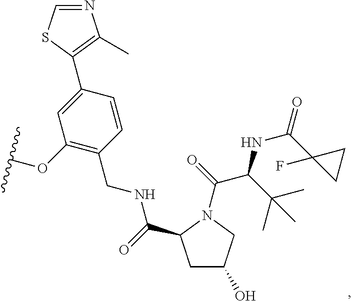 [SMILES: *Oc1cc(-c2scnc2C)ccc1CNC(=O)[C@@H]1C[C@@H](O)CN1C(=O)[C@@H](NC(=O)C1(F)CC1)C(C)(C)C] | (s) |
| 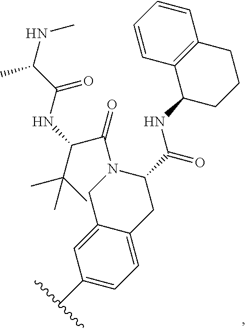 [SMILES: *c1ccc2c(c1)CN(C(=O)[C@@H](NC(=O)[C@H](C)NC)C(C)(C)C)[C@H](C(=O)N[C@@H]1CCCc3ccccc31)C2] | (t) |
| 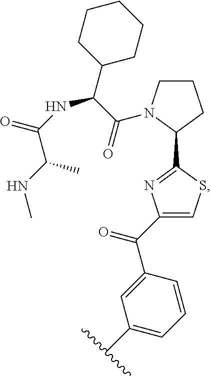 [SMILES: *c1cccc(C(=O)c2csc([C@@H]3CCCN3C(=O)[C@@H](NC(=O)[C@H](C)NC)C3CCCCC3)n2)c1] | (u) |
| 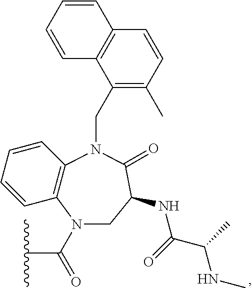 [SMILES: *C(=O)N1C[C@H](NC(=O)[C@H](C)NC)C(=O)N(Cc2c(C)ccc3ccccc23)c2ccccc21] | (v) |
| 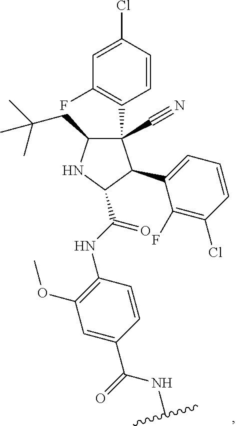 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1] | (w) |
| 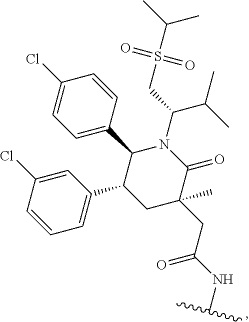 [SMILES: *NC(=O)C[C@@]1(C)C[C@H](c2cccc(Cl)c2)[C@@H](c2ccc(Cl)cc2)N([C@H](CS(=O)(=O)C(C)C)C(C)C)C1=O] | (x) |
| 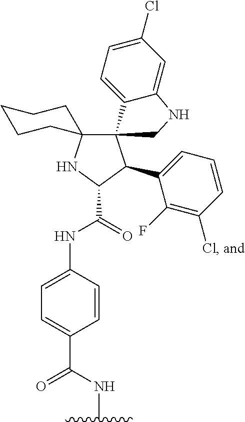 | (y) |
| 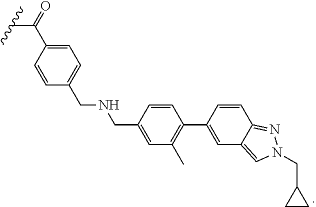 [SMILES: *C(=O)c1ccc(CNCc2ccc(-c3ccc4nn(CC5CC5)cc4c3)c(C)c2)cc1] | (z) |

TABLE B
Exemplified Linkers (L)

| | |
|---|---|
| 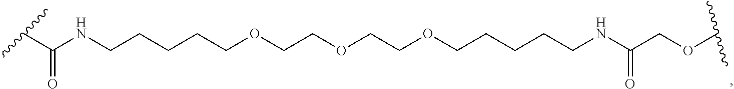 [SMILES: *OCC(=O)NCCCCCOCCOCCOCCCCCNC(*)=O] | (1) |
| 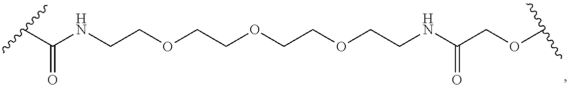 [SMILES: *OCC(=O)NCCOCCOCCOCCNC(*)=O] | (2) |
| 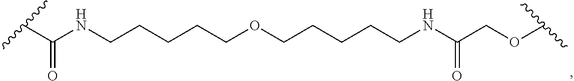 [SMILES: *OCC(=O)NCCCCCOCCCCCNC(*)=O] | (3) |
| 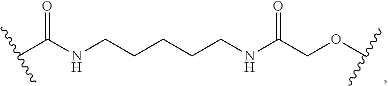 [SMILES: *OCC(=O)NCCCCCNC(*)=O] | (4) |
| 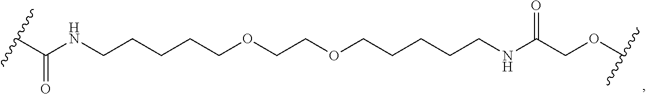 [SMILES: *OCC(=O)NCCCCCOCCOCCCCCNC(*)=O] | (5) |
| 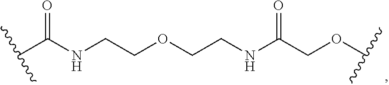 [SMILES: *OCC(=O)NCCOCCNC(*)=O] | (6) |
| 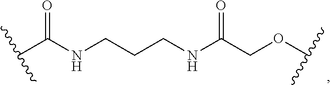 [SMILES: *OCC(=O)NCCCNC(*)=O] | (7) |
| 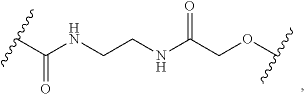 [SMILES: *OCC(=O)NCCNC(*)=O] | (8) |
| 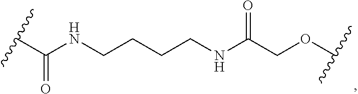 [SMILES: *OCC(=O)NCCCCNC(*)=O] | (9) |
| 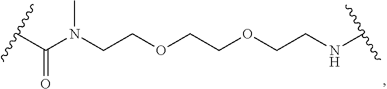 [SMILES: *NCCOCCOCCN(C)C(*)=O] | (10) |
| 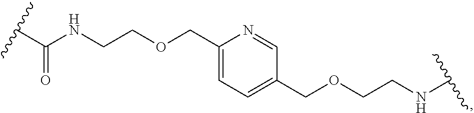 [SMILES: *NCCOCc1ccc(COCCNC(*)=O)nc1] | (11) |
| 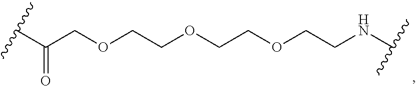 [SMILES: *NCCOCCOCCOCC(*)=O] | (12) |
| 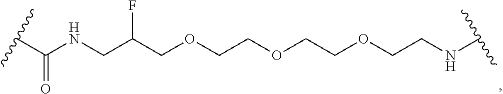 [SMILES: *NCCOCCOCCOCC(F)CNC(*)=O] | (13) |
| 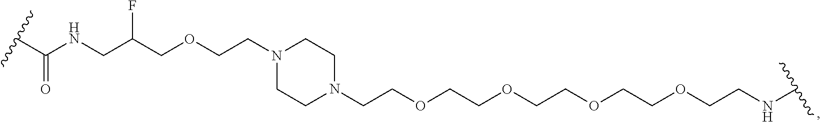 [SMILES: *NCCOCCOCCOCCOCCN1CCN(CCOCC(F)CNC(*)=O)CC1] | (14) |
| 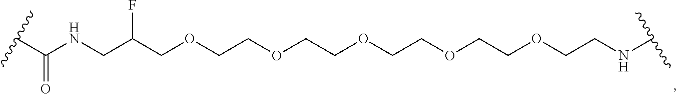 [SMILES: *NCCOCCOCCOCCOCCOCC(F)CNC(*)=O] | (15) |
| 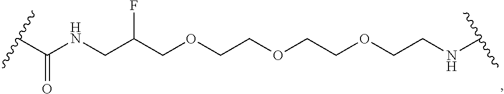 [SMILES: *NCCOCCOCCOCC(F)CNC(*)=O] | (16) |
| 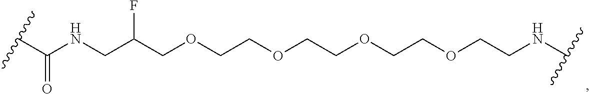 [SMILES: *NCCOCCOCCOCCOCC(F)CNC(*)=O] | (17) |
| 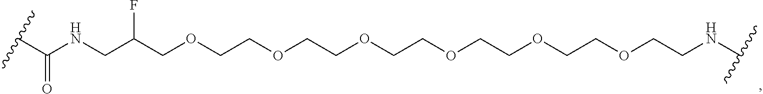 [SMILES: *NCCOCCOCCOCCOCCOCCOCC(F)CNC(*)=O] | (18) |
| 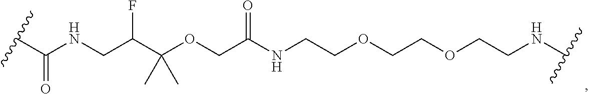 [SMILES: *NCCOCCOCCNC(=O)COC(C)(C)C(F)CNC(*)=O] | (19) |
| 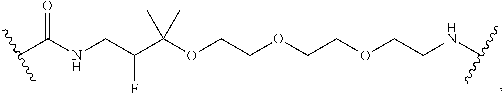 [SMILES: *NCCOCCOCCOC(C)(C)C(F)CNC(*)=O] | (20) |
| 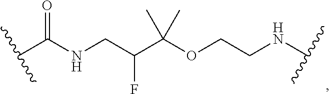 [SMILES: *NCCOC(C)(C)C(F)CNC(*)=O] | (21) |
| 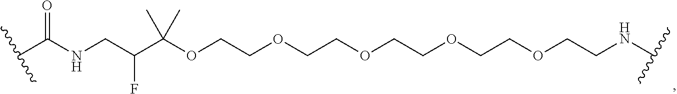 [SMILES: *NCCOCCOCCOCCOCCOC(C)(C)C(F)CNC(*)=O] | (22) |
| 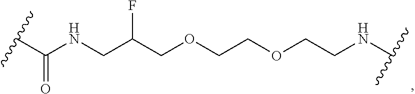 [SMILES: *NCCOCCOCC(F)CNC(*)=O] | (23) |
| 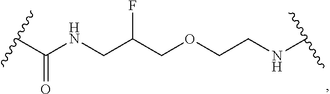 [SMILES: *NCCOCC(F)CNC(*)=O] | (24) |
| 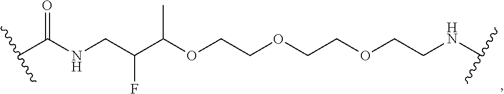 [SMILES: *NCCOCCOCCOC(C)C(F)CNC(*)=O] | (25) |
| 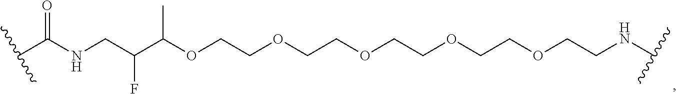 [SMILES: *NCCOCCOCCOCCOCCOC(C)C(F)CNC(*)=O] | (26) |
| 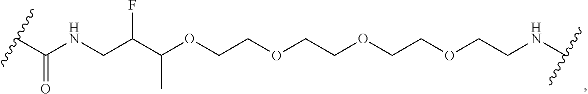 [SMILES: *NCCOCCOCCOCCOC(C)C(F)CNC(*)=O] | (27) |
| 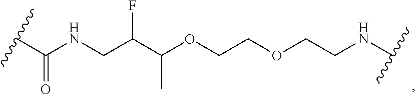 [SMILES: *NCCOCCOC(C)C(F)CNC(*)=O] | (28) |
| 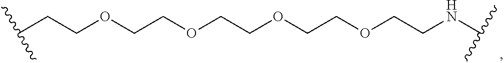 [SMILES: *CCOCCOCCOCCOCCN*] | (29) |
| 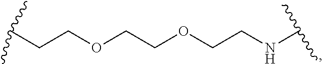 [SMILES: *CCOCCOCCN*] | (30) |
| 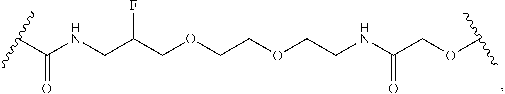 [SMILES: *OCC(=O)NCCOCCOCC(F)CNC(*)=O] | (31) |
| 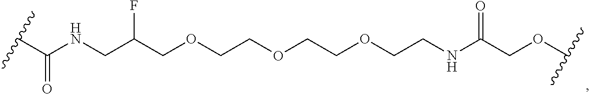 [SMILES: *OCC(=O)NCCOCCOCCOCC(F)CNC(*)=O] | (32) |
| 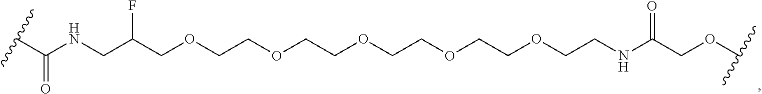 [SMILES: *OCC(=O)NCCOCCOCCOCCOCCOCC(F)CNC(*)=O] | (33) |
| 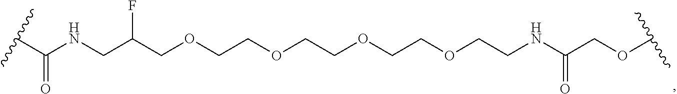 [SMILES: *OCC(=O)NCCOCCOCCOCCOCC(F)CNC(*)=O] | (34) |
| 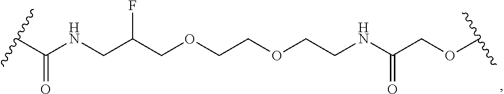 [SMILES: *OCC(=O)NCCOCCOCC(F)CNC(*)=O] | (35) |
| 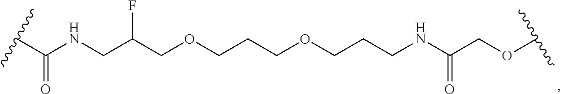 [SMILES: *OCC(=O)NCCCOCCCOCC(F)CNC(*)=O] | (36) |
| 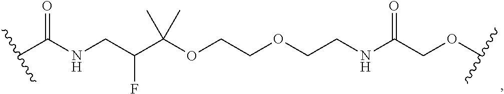 [SMILES: *OCC(=O)NCCOCCOC(C)(C)C(F)CNC(*)=O] | (37) |
| 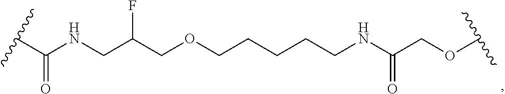 [SMILES: *OCC(=O)NCCCCCOCC(F)CNC(*)=O] | (38) |
| 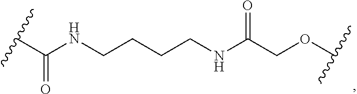 [SMILES: *OCC(=O)NCCCCNC(*)=O] | (39) |
| 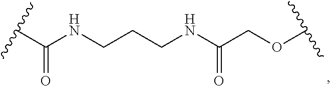 [SMILES: *OCC(=O)NCCCNC(*)=O] | (40) |
| 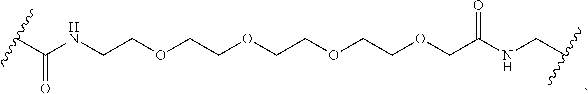 [SMILES: *CNC(=O)COCCOCCOCCOCCNC(*)=O] | (41) |
| 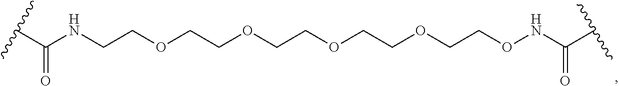 [SMILES: *C(=O)NCCOCCOCCOCCOCCONC(*)=O] | (42) |
| 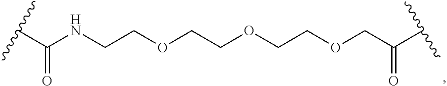 [SMILES: *C(=O)COCCOCCOCCNC(*)=O] | (43) |
| 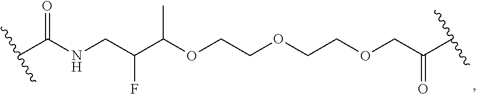 [SMILES: *C(=O)COCCOCCOC(C)C(F)CNC(*)=O] | (44) |
| 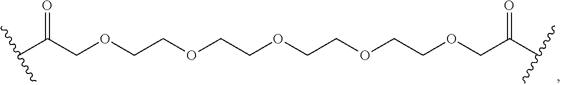 [SMILES: *C(=O)COCCOCCOCCOCCOCC(*)=O] | (45) |
| 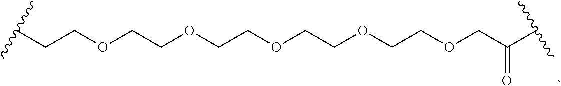 [SMILES: *CCOCCOCCOCCOCCOCC(*)=O] | (46) |
| 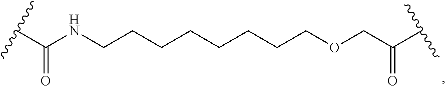 [SMILES: *C(=O)COCCCCCCCCNC(*)=O] | (47) |
| 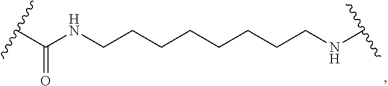 [SMILES: *NCCCCCCCCNC(*)=O] | (49) |
| 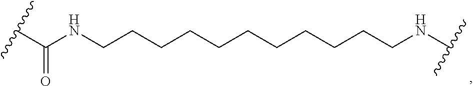 [SMILES: *NCCCCCCCCCCCNC(*)=O] | (50) |
| 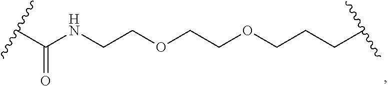 [SMILES: *CCCOCCOCCNC(*)=O] | (51) |
| 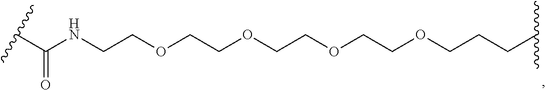 [SMILES: *CCCOCCOCCOCCOCCNC(*)=O] | (52) |
| 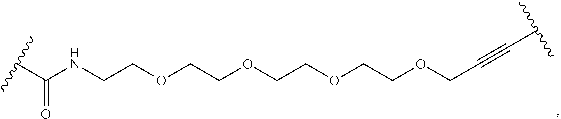 [SMILES: *C#CCOCCOCCOCCOCCNC(*)=O] | (53) |
| 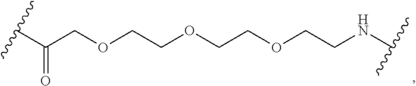 [SMILES: *NCCOCCOCCOCC(*)=O] | (54) |
| 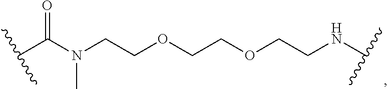 [SMILES: *NCCOCCOCCN(C)C(*)=O] | (55) |
| 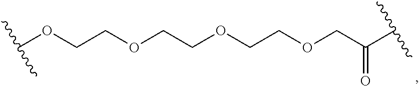 [SMILES: *OCCOCCOCCOCC(*)=O] | (56) |
| 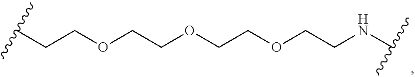 [SMILES: *CCOCCOCCOCCN*] | (57) |
| 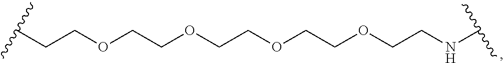 [SMILES: *CCOCCOCCOCCOCCN*] | (58) |
| 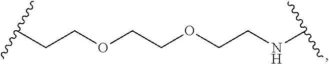 [SMILES: *CCOCCOCCN*] | (59) |
| 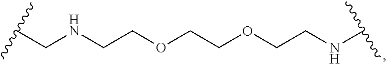 [SMILES: *CNCCOCCOCCN*] | (60) |
| 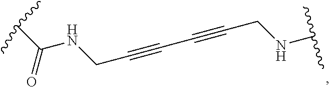 [SMILES: *NCC#CC#CCNC(*)=O] | (61) |
| 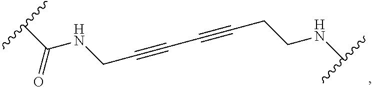 [SMILES: *NCCC#CC#CCNC(*)=O] | (62) |
| 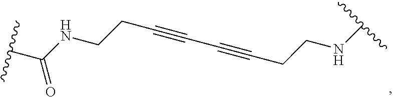 [SMILES: *NCCC#CC#CCCNC(*)=O] | (63) |
| 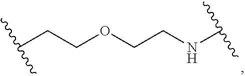 [SMILES: *CCOCCN*] | (64) |
| 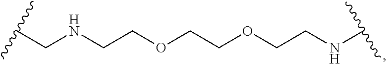 [SMILES: *CNCCOCCOCCN*] | (65) |
| 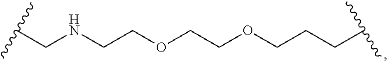 [SMILES: *CCCOCCOCCNC*] | (66) |
| 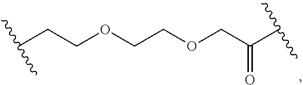 [SMILES: *CCOCCOCC(*)=O] | (67) |
| 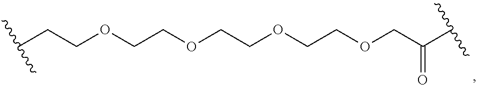 [SMILES: *CCOCCOCCOCCOCC(*)=O] | (68) |
| 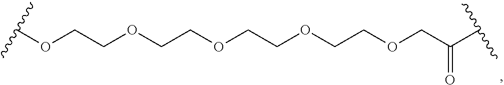 [SMILES: *OCCOCCOCCOCCOCC(*)=O] | (69) |
| 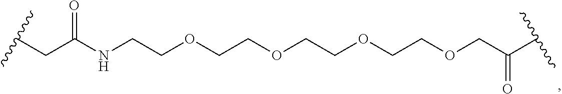 [SMILES: *CC(=O)NCCOCCOCCOCCOCC(*)=O] | (70) |
| 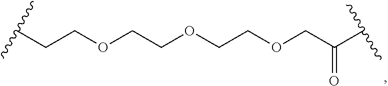 [SMILES: *CCOCCOCCOCC(*)=O] | (71) |
| 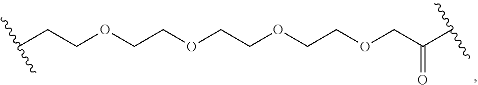 [SMILES: *CCOCCOCCOCCOCC(*)=O] | (72) |
| 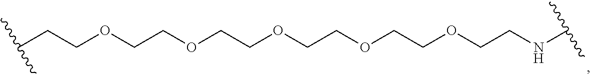 [SMILES: *CCOCCOCCOCCOCCOCCN*] | (73) |
| 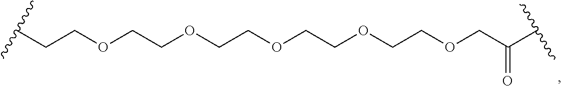 [SMILES: *CCOCCOCCOCCOCCOCC(*)=O] | (74) |
| 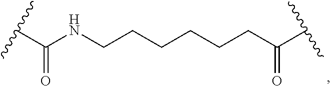 [SMILES: *C(=O)CCCCCCNC(*)=O] | (75) |
| 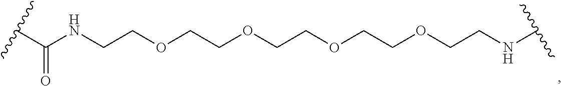 [SMILES: *NCCOCCOCCOCCOCCNC(*)=O] | (76) |
| 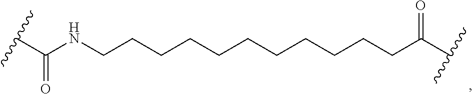 [SMILES: *C(=O)CCCCCCCCCCCNC(*)=O] | (77) |
| 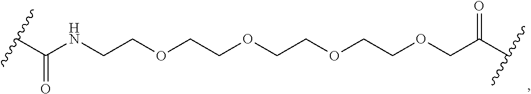 [SMILES: *C(=O)COCCOCCOCCOCCNC(*)=O] | (78) |
| 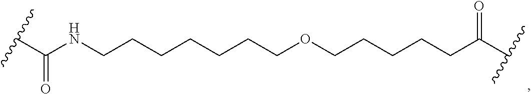 [SMILES: *C(=O)CCCCCOCCCCCCCNC(*)=O] | (79) |
| 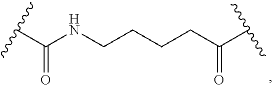 [SMILES: *C(=O)CCCCNC(*)=O] | (80) |
| 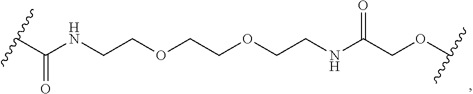 [SMILES: *OCC(=O)NCCOCCOCCNC(*)=O] | (81) |
| 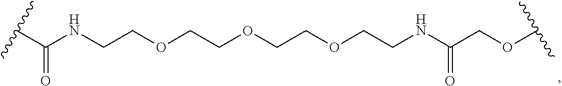 [SMILES: *OCC(=O)NCCOCCOCCOCCNC(*)=O] | (82) |
| 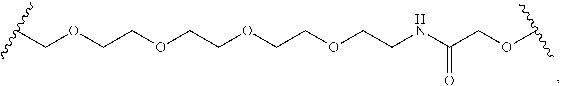 [SMILES: *COCCOCCOCCOCCNC(=O)CO*] | (83) |
| 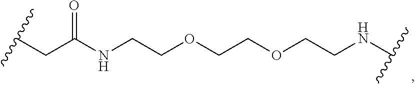 [SMILES: *CC(=O)NCCOCCOCCN*] | (84) |
| 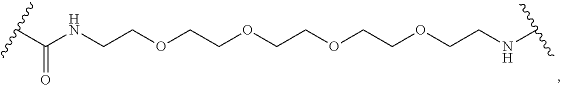 [SMILES: *NCCOCCOCCOCCOCCNC(*)=O] | (85) |
| 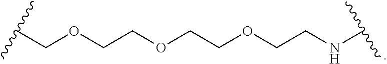 [SMILES: *COCCOCCOCCN*] | (86) |
| 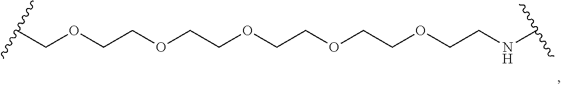 [SMILES: *COCCOCCOCCOCCOCCN*] | (87) |
| 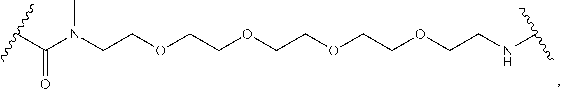 [SMILES: *NCCOCCOCCOCCOCCN(C)C(*)=O] | (88) |
| 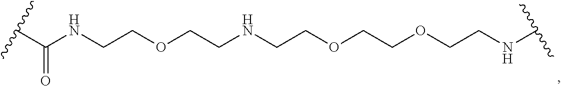 [SMILES: *NCCOCCOCCNCCOCCNC(*)=O] | (89) |
| 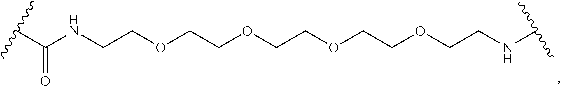 [SMILES: *NCCOCCOCCOCCOCCNC(*)=O] | (90) |
| 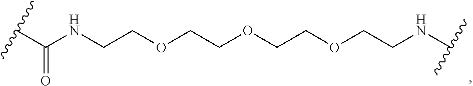 [SMILES: *NCCOCCOCCOCCNC(*)=O] | (91) |
| 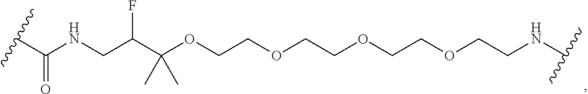 [SMILES: *NCCOCCOCCOCCOC(C)(C)C(F)CNC(*)=O] | (92) |
| 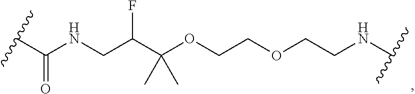 [SMILES: *NCCOCCOC(C)(C)C(F)CNC(*)=O] | (93) |
| 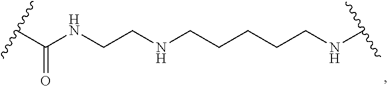 [SMILES: *NCCCCCNCCNC(*)=O] | (94) |
| 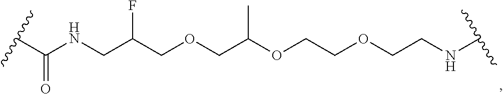 [SMILES: *NCCOCCOC(C)COCC(F)CNC(*)=O] | (95) |
| 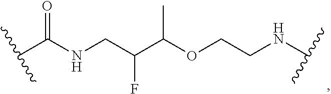 [SMILES: *NCCOC(C)C(F)CNC(*)=O] | (96) |
| 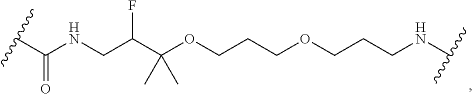 [SMILES: *NCCCOCCCOC(C)(C)C(F)CNC(*)=O] | (97) |
| 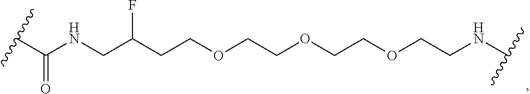 [SMILES: *NCCOCCOCCOCCC(F)CNC(*)=O] | (98) |
| 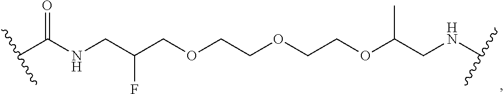 [SMILES: *NCC(C)OCCOCCOCC(F)CNC(*)=O] | (99) |
| 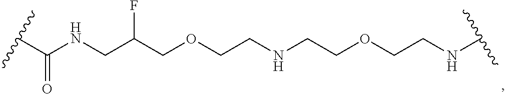 [SMILES: *NCCOCCNCCOCC(F)CNC(*)=O] | (100) |
| 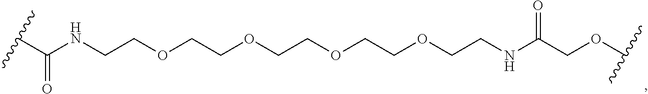 [SMILES: *OCC(=O)NCCOCCOCCOCCOCCNC(*)=O] | (101) |
| 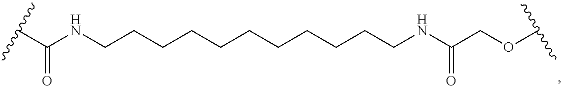 [SMILES: *OCC(=O)NCCCCCCCCCCCNC(*)=O] | (102) |
| 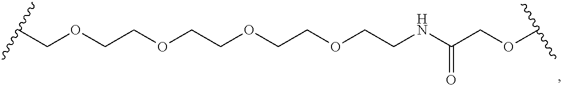 [SMILES: *COCCOCCOCCOCCNC(=O)CO*] | (103) |
| 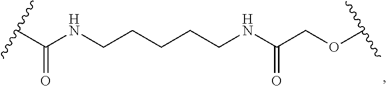 [SMILES: *OCC(=O)NCCCCCNC(*)=O] | (104) |
| 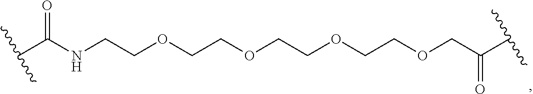 [SMILES: *C(=O)COCCOCCOCCOCCNC(*)=O] | (105) |
| 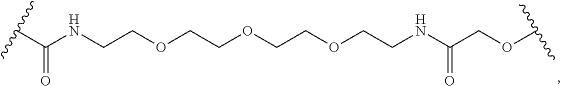 [SMILES: *OCC(=O)NCCOCCOCCOCCNC(*)=O] | (106) |
| 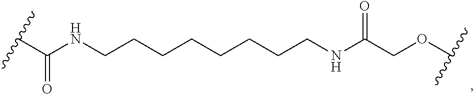 [SMILES: *OCC(=O)NCCCCCCCCNC(*)=O] | (107) |
| 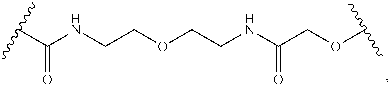 [SMILES: *OCC(=O)NCCOCCNC(*)=O] | (108) |
| 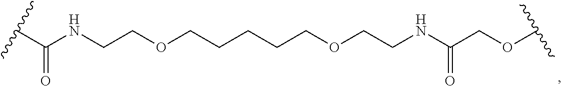 [SMILES: *OCC(=O)NCCOCCCCCOCCNC(*)=O] | (109) |
| 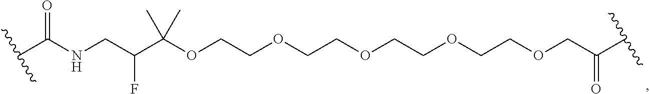 [SMILES: *C(=O)COCCOCCOCCOCCOC(C)(C)C(F)CNC(*)=O] | (110) |
| 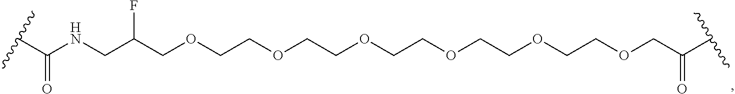 [SMILES: *C(=O)COCCOCCOCCOCCOCCOCC(F)CNC(*)=O] | (111) |
| 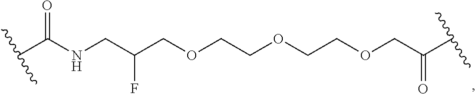 [SMILES: *C(=O)COCCOCCOCC(F)CNC(*)=O] | (112) |
| 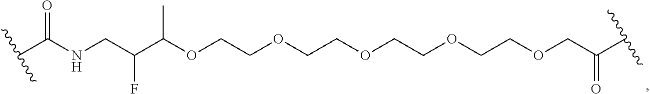 [SMILES: *C(=O)COCCOCCOCCOCCOC(C)C(F)CNC(*)=O] | (113) |
| 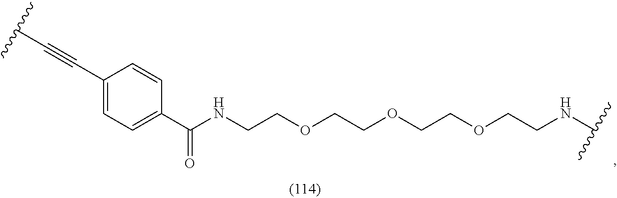 [SMILES: *C#Cc1ccc(C(=O)NCCOCCOCCOCCN*)cc1] | (114) |
| 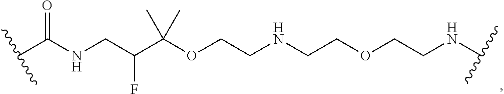 [SMILES: *NCCOCCNCCOC(C)(C)C(F)CNC(*)=O] | (115) |
| 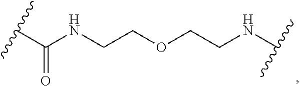 [SMILES: *NCCOCCNC(*)=O] | (116) |
| 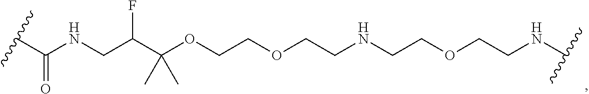 [SMILES: *NCCOCCNCCOCCOC(C)(C)C(F)CNC(*)=O] | (117) |
| 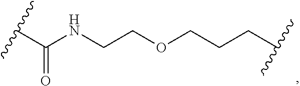 [SMILES: *CCCOCCNC(*)=O] | (118) |
| 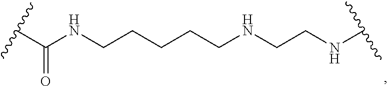 [SMILES: *NCCNCCCCCNC(*)=O] | |
| 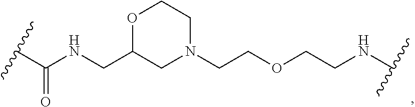 [SMILES: *NCCOCCN1CCOC(CNC(*)=O)C1] | (119) |
| 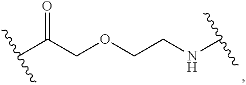 [SMILES: *NCCOCC(*)=O] | |
| 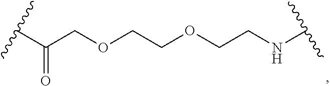 [SMILES: *NCCOCCOCC(*)=O] | (120) |
| 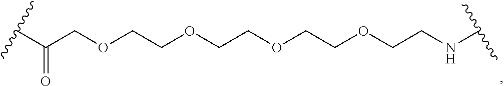 [SMILES: *NCCOCCOCCOCCOCC(*)=O] | (121) |
| 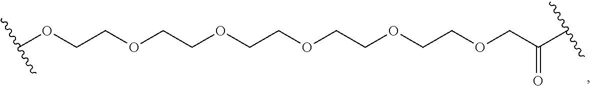 [SMILES: *OCCOCCOCCOCCOCCOCC(*)=O] | (122) |
| 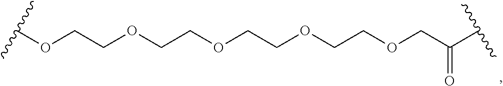 [SMILES: *OCCOCCOCCOCCOCC(*)=O] | (123) |
| 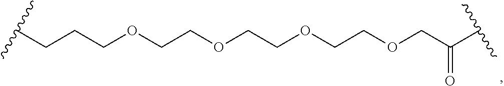 [SMILES: *CCCOCCOCCOCCOCC(*)=O] | (124) |
| 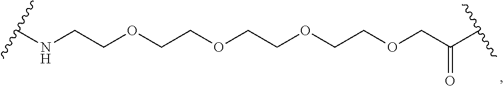 [SMILES: *NCCOCCOCCOCCOCC(*)=O] | (125) |
| 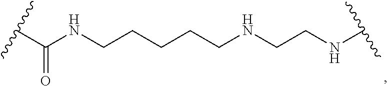 [SMILES: *NCCNCCCCCNC(*)=O] | (126) |
| 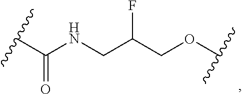 [SMILES: *OCC(F)CNC(*)=O] | |
| 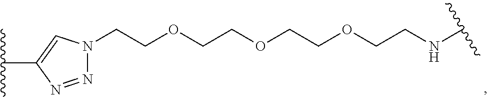 [SMILES: *NCCOCCOCCOCCn1cc(*)nn1] | (127) |
| 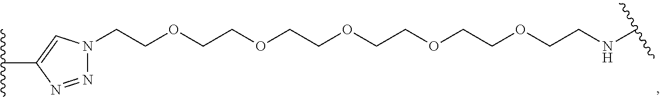 [SMILES: *NCCOCCOCCOCCOCCOCCn1cc(*)nn1] | (128) |
| 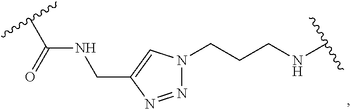 [SMILES: *NCCCn1cc(CNC(*)=O)nn1] | (129) |
| 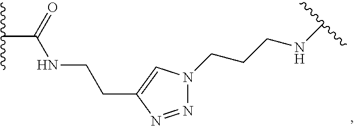 [SMILES: *NCCCn1cc(CCNC(*)=O)nn1] | (130) |
| 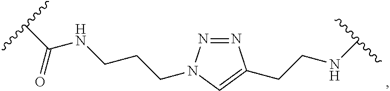 [SMILES: *NCCc1cn(CCCNC(*)=O)nn1] | (131) |
| 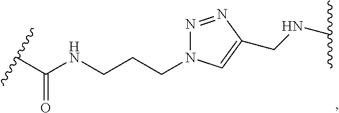 [SMILES: *NCc1cn(CCCNC(*)=O)nn1] | (132) |
| 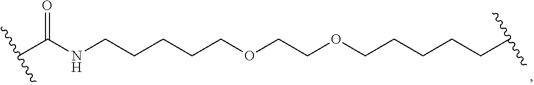 [SMILES: *CCCCCOCCOCCCCCNC(*)=O] | (133) |
| 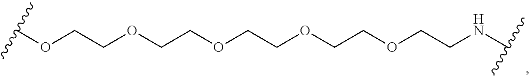 [SMILES: *NCCOCCOCCOCCOCCO*] | (134) |
| 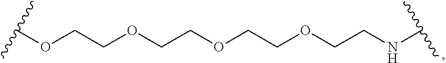 [SMILES: *NCCOCCOCCOCCO*] | (135) |
| 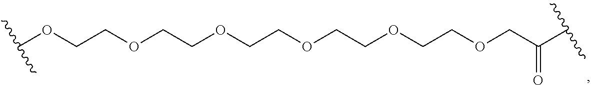 [SMILES: *OCCOCCOCCOCCOCCOCC(*)=O] | (136) |
| 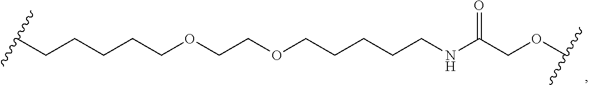 [SMILES: *CCCCCOCCOCCCCCNC(=O)CO*] | (137) |
| 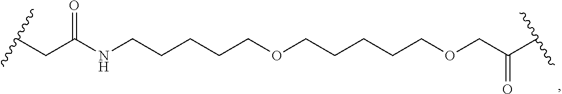 [SMILES: *CC(=O)NCCCCCOCCCCCOCC(*)=O] | (138) |
| 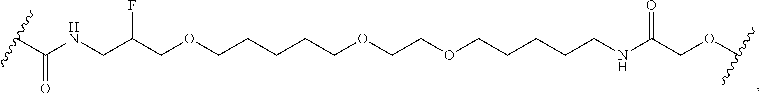 [SMILES: *OCC(=O)NCCCCCOCCOCCCCCOCC(F)CNC(*)=O] | (139) |
| 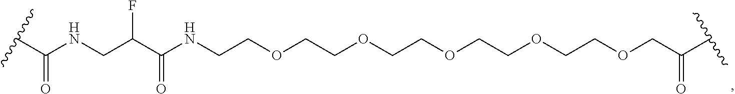 [SMILES: *C(=O)COCCOCCOCCOCCOCCNC(=O)C(F)CNC(*)=O] | (140) |
| 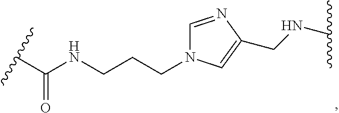 [SMILES: *NCc1cn(CCCNC(*)=O)cn1] | (141) |
| 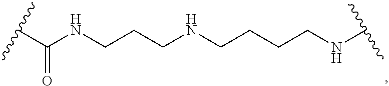 [SMILES: *NCCCCNCCCNC(*)=O] | (142) |
| 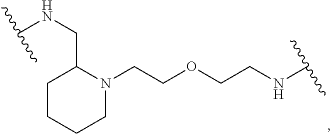 [SMILES: *NCCOCCN1CCCCC1CN*] | (143) |
| 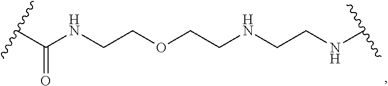 [SMILES: *NCCNCCOCCNC(*)=O] | (144) |
| 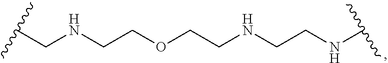 [SMILES: *CNCCOCCNCCN*] | (145) |
| 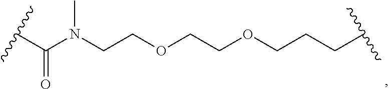 [SMILES: *CCCOCCOCCN(C)C(*)=O] | (146) |
| 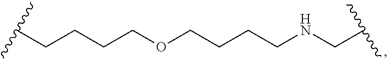 [SMILES: *CCCCOCCCCNC*] | (147) |
| 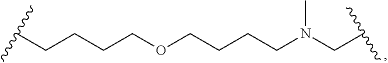 [SMILES: *CCCCOCCCCN(C)C*] | (148) |
| 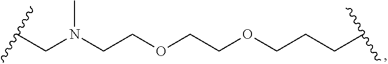 [SMILES: *CCCOCCOCCN(C)C*] | (149) |
| 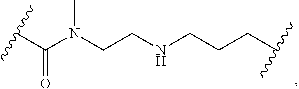 [SMILES: *CCCNCCN(C)C(*)=O] | (150) |
| 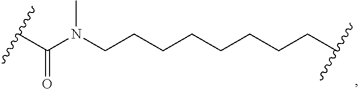 [SMILES: *CCCCCCCCN(C)C(*)=O] | (151) |
| 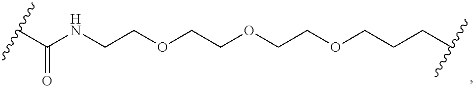 [SMILES: *CCCOCCOCCOCCNC(*)=O] | (152) |
| 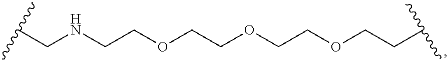 [SMILES: *CCOCCOCCOCCNC*] | (153) |
| 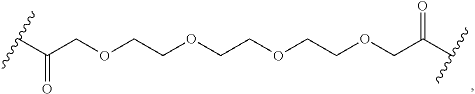 [SMILES: *C(=O)COCCOCCOCCOCC(*)=O] | (154) |
| 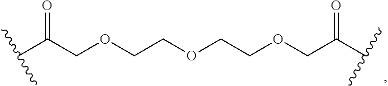 [SMILES: *C(=O)COCCOCCOCC(*)=O] | (155) |
| 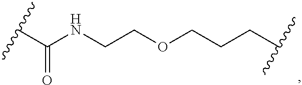 [SMILES: *CCCOCCNC(*)=O] | (156) |
| 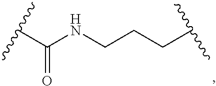 [SMILES: *CCCNC(*)=O] | (157) |
| 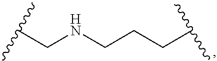 [SMILES: *CCCNC*] | (158) |
| 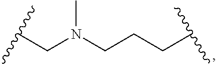 [SMILES: *CCCN(C)C*] | (159) |
| 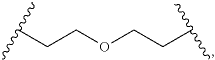 [SMILES: *CCOCC*] | (160) |
| 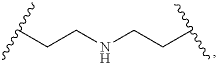 [SMILES: *CCNCC*] | (161) |
| 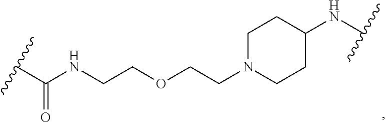 [SMILES: *NC1CCN(CCOCCNC(*)=O)CC1] | (162) |
| 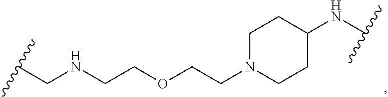 [SMILES: *CNCCOCCN1CCC(N*)CC1] | (163) |
| 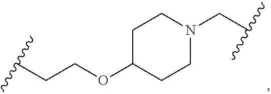 [SMILES: *CCOC1CCN(C*)CC1] | (164) |
| 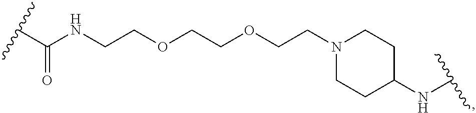 [SMILES: *NC1CCN(CCOCCOCCNC(*)=O)CC1] | (165) |
| 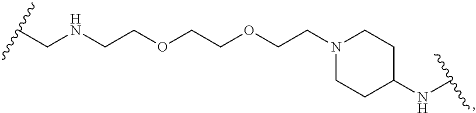 [SMILES: *CNCCOCCOCCN1CCC(N*)CC1] | (166) |
| 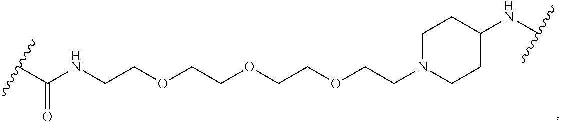 [SMILES: *NC1CCN(CCOCCOCCOCCNC(*)=O)CC1] | (167) |
| 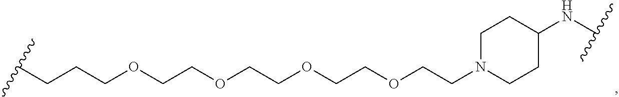 [SMILES: *CCCOCCOCCOCCOCCN1CCC(N*)CC1] | (168) |
| 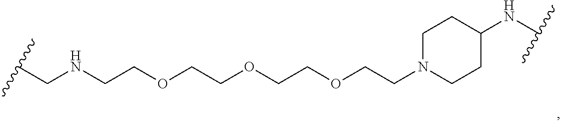 [SMILES: *CNCCOCCOCCOCCN1CCC(N*)CC1] | (169) |
| 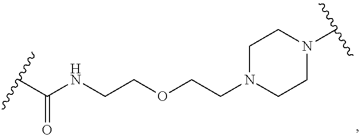 [SMILES: *C(=O)NCCOCCN1CCN(*)CC1] | (170) |
| 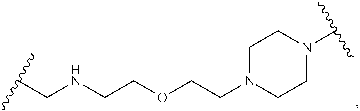 [SMILES: *CNCCOCCN1CCN(*)CC1] | (171) |
| 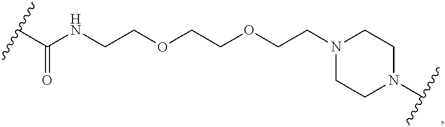 [SMILES: *C(=O)NCCOCCOCCN1CCN(*)CC1] | (172) |
| 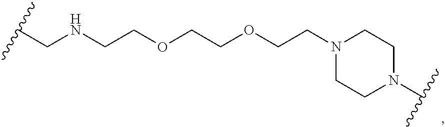 [SMILES: *CNCCOCCOCCN1CCN(*)CC1] | (173) |
| 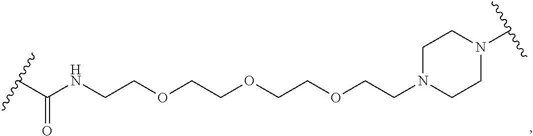 [SMILES: *C(=O)NCCOCCOCCOCCN1CCN(*)CC1] | (174) |
| 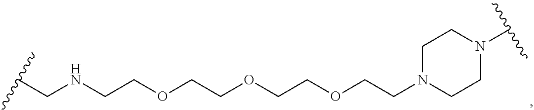 [SMILES: *CNCCOCCOCCOCCN1CCN(*)CC1] | (175) |
| 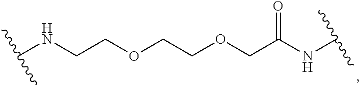 [SMILES: *NCCOCCOCC(=O)N*] | (176) |
| 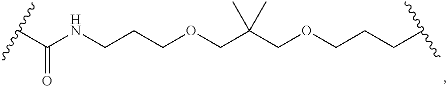 [SMILES: *CCCOCC(C)(C)COCCCNC(*)=O] | (177) |
| 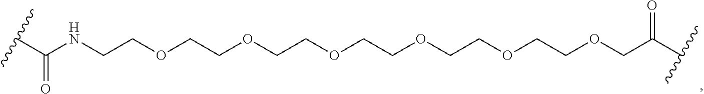 [SMILES: *C(=O)COCCOCCOCCOCCOCCOCCNC(*)=O] | (178) |
| 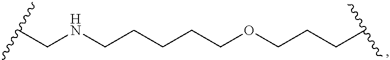 [SMILES: *CCCOCCCCCNC*] | (179) |
| 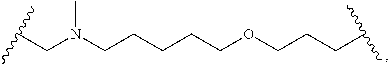 [SMILES: *CCCOCCCCCN(C)C*] | (180) |
| 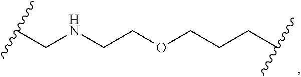 [SMILES: *CCCOCCNC*] | (181) |
| 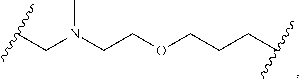 [SMILES: *CCCOCCN(C)C*] | (182) |
| 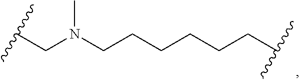 [SMILES: *CCCCCCN(C)C*] | (183) |
| 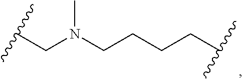 [SMILES: *CCCCN(C)C*] | (184) |
| 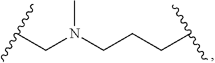 [SMILES: *CCCN(C)C*] | (185) |
| 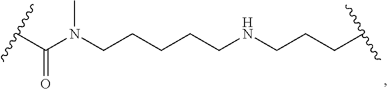 [SMILES: *CCCNCCCCCN(C)C(*)=O] | (186) |
| 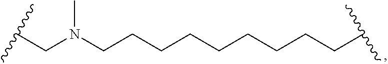 [SMILES: *CCCCCCCCCN(C)C*] | (187) |
| 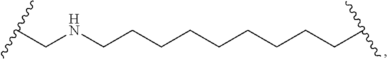 [SMILES: *CCCCCCCCCNC*] | (188) |
| 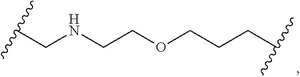 [SMILES: *CCCOCCNC*] | (189) |
| 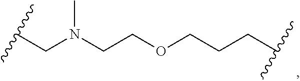 [SMILES: *CCCOCCN(C)C*] | (190) |
| 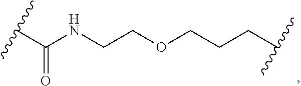 [SMILES: *CCCOCCNC(*)=O] | (191) |
| 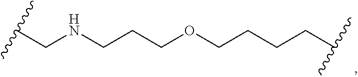 [SMILES: *CCCCOCCCNC*] | (192) |
| 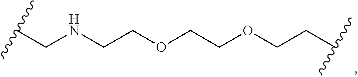 [SMILES: *CCOCCOCCNC*] | (193) |
| 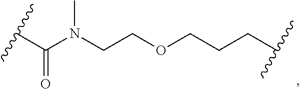 [SMILES: *CCCOCCN(C)C(*)=O] | (194) |
| 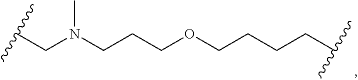 [SMILES: *CCCCOCCCN(C)C*] | (195) |
| 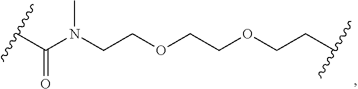 [SMILES: *CCOCCOCCN(C)C(*)=O] | (196) |
| 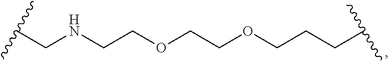 [SMILES: *CCCOCCOCCNC*] | (197) |
| 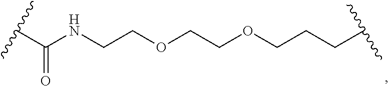 [SMILES: *CCCOCCOCCNC(*)=O] | (198) |
| 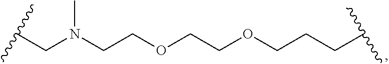 [SMILES: *CCCOCCOCCN(C)C*] | (199) |
| 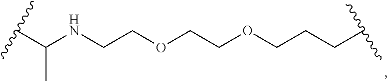 [SMILES: *CCCOCCOCCNC(*)C] | (200) |
| 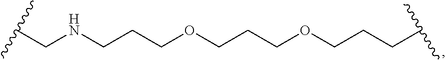 [SMILES: *CCCOCCCOCCCNC*] | (201) |
| 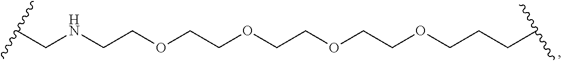 [SMILES: *CCCOCCOCCOCCOCCNC*] | (202) |
| 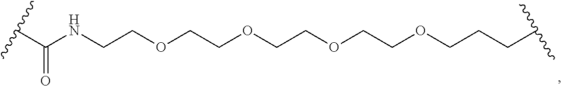 [SMILES: *CCCOCCOCCOCCOCCNC(*)=O] | (203) |
| 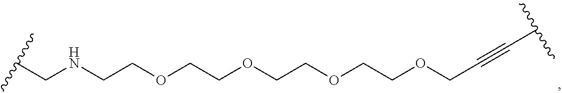 [SMILES: *C#CCOCCOCCOCCOCCNC*] | (204) |
| 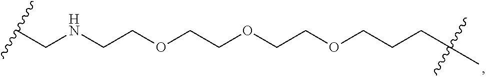 [SMILES: *CCCOCCOCCOCCNC*] | (205) |
| 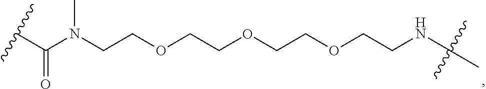 [SMILES: *NCCOCCOCCOCCN(C)C(*)=O] | (206) |
| 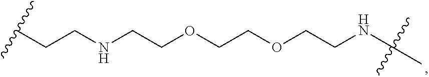 [SMILES: *CCNCCOCCOCCN*] | (207) |
| 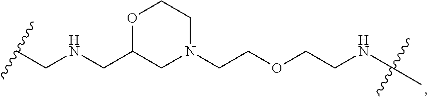 [SMILES: *CNCC1CN(CCOCCN*)CCO1] | (208) |
| 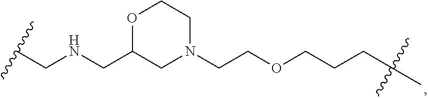 [SMILES: *CCCOCCN1CCOC(CNC*)C1] | (209) |
| 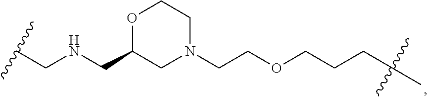 [SMILES: *CCCOCCN1CCO[C@H](CNC*)C1] | (210) |
| 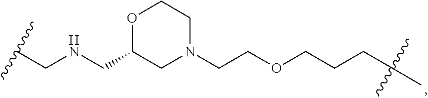 [SMILES: *CCCOCCN1CCO[C@@H](CNC*)C1] | (211) |
| 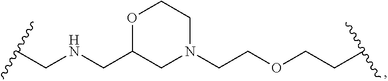 [SMILES: *CCOCCN1CCOC(CNC*)C1] | (212) |
| 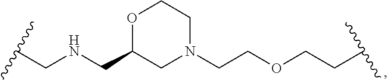 [SMILES: *CCOCCN1CCO[C@H](CNC*)C1] | (213) |
| 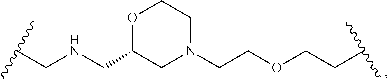 [SMILES: *CCOCCN1CCO[C@@H](CNC*)C1] | (214) |
| 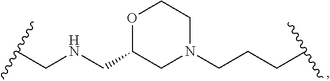 [SMILES: *CCCN1CCO[C@@H](CNC*)C1] | (215) |
| 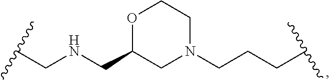 [SMILES: *CCCN1CCO[C@H](CNC*)C1] | (216) |
| 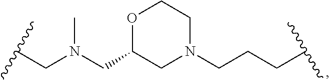 [SMILES: *CCCN1CCO[C@@H](CN(C)C*)C1] | (217) |
| 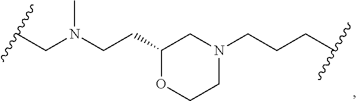 [SMILES: *CCCN1CCO[C@H](CCN(C)C*)C1] | (218) |
| 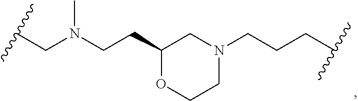 [SMILES: *CCCN1CCO[C@@H](CCN(C)C*)C1] | (219) |
| 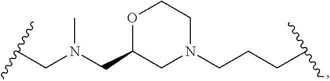 [SMILES: *CCCN1CCO[C@H](CN(C)C*)C1] | (220) |
| 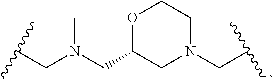 [SMILES: *CN(C)C[C@H]1CN(C*)CCO1] | (221) |
| 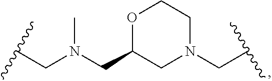 [SMILES: *CN(C)C[C@@H]1CN(C*)CCO1] | (222) |
| 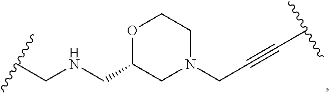 [SMILES: *C#CCN1CCO[C@@H](CNC*)C1] | (223) |
| 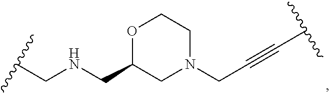 [SMILES: *C#CCN1CCO[C@H](CNC*)C1] | (224) |
| 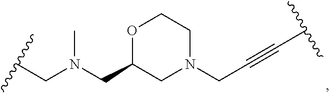 [SMILES: *C#CCN1CCO[C@H](CN(C)C*)C1] | (225) |
| 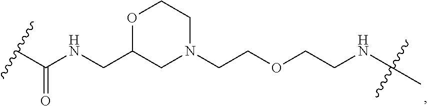 [SMILES: *NCCOCCN1CCOC(CNC(*)=O)C1] | (226) |
| 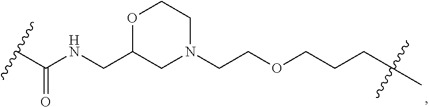 [SMILES: *CCCOCCN1CCOC(CNC(*)=O)C1] | (227) |
| 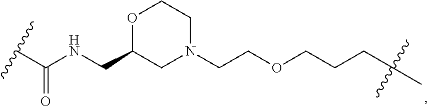 [SMILES: *CCCOCCN1CCO[C@H](CNC(*)=O)C1] | (228) |
| 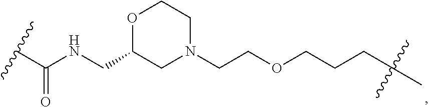 [SMILES: *CCCOCCN1CCO[C@@H](CNC(*)=O)C1] | (229) |
| 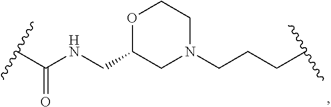 [SMILES: *CCCN1CCO[C@@H](CNC(*)=O)C1] | (230) |
| 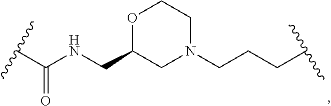 [SMILES: *CCCN1CCO[C@H](CNC(*)=O)C1] | (231) |
| 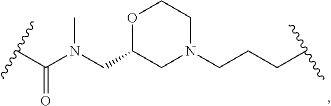 [SMILES: *CCCN1CCO[C@@H](CN(C)C(*)=O)C1] | (232) |
| 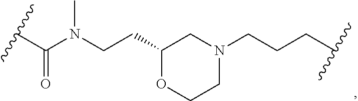 [SMILES: *CCCN1CCO[C@H](CCN(C)C(*)=O)C1] | (233) |
| 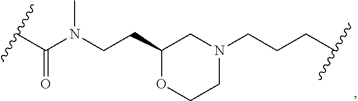 [SMILES: *CCCN1CCO[C@@H](CCN(C)C(*)=O)C1] | (234) |
| 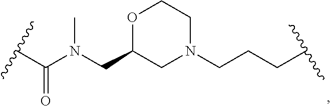 [SMILES: *CCCN1CCO[C@H](CN(C)C(*)=O)C1] | (235) |
| 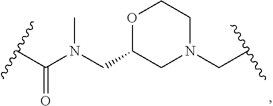 [SMILES: *CN1CCO[C@@H](CN(C)C(*)=O)C1] | (236) |
| 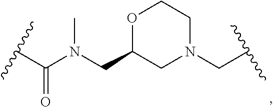 [SMILES: *CN1CCO[C@H](CN(C)C(*)=O)C1] | (237) |
| 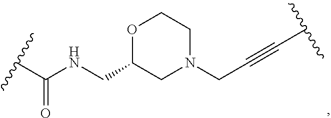 [SMILES: *C#CCN1CCO[C@@H](CNC(*)=O)C1] | (238) |
| 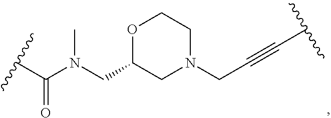 [SMILES: *C#CCN1CCO[C@@H](CN(C)C(*)=O)C1] | (239) |
| 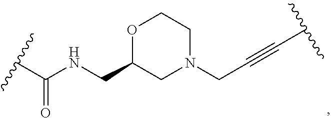 [SMILES: *C#CCN1CCO[C@H](CNC(*)=O)C1] | (240) |
| 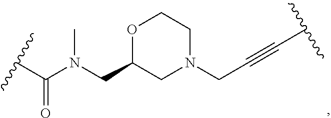 [SMILES: *C#CCN1CCO[C@H](CN(C)C(*)=O)C1] | (241) |
| 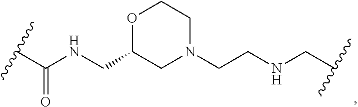 [SMILES: *CNCCN1CCO[C@@H](CNC(*)=O)C1] | (242) |
| 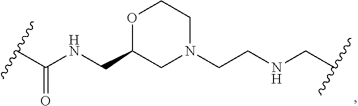 [SMILES: *CNCCN1CCO[C@H](CNC(*)=O)C1] | (243) |
| 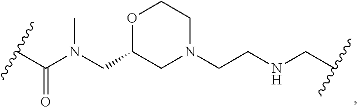 [SMILES: *CNCCN1CCO[C@@H](CN(C)C(*)=O)C1] | (244) |
| 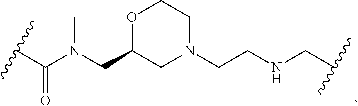 [SMILES: *CNCCN1CCO[C@H](CN(C)C(*)=O)C1] | (245) |
| 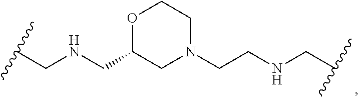 [SMILES: *CNCCN1CCO[C@@H](CNC*)C1] | (246) |
| 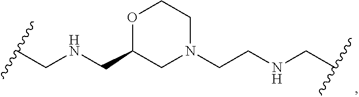 [SMILES: *CNCCN1CCO[C@H](CNC*)C1] | (247) |
| 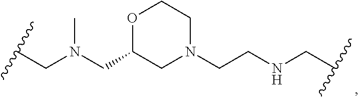 [SMILES: *CNCCN1CCO[C@@H](CN(C)C*)C1] | (248) |
| 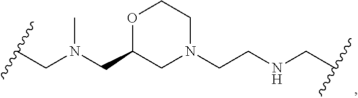 [SMILES: *CNCCN1CCO[C@H](CN(C)C*)C1] | (249) |
| 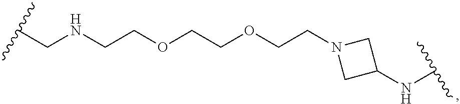 [SMILES: *CNCCOCCOCCN1CC(N*)C1] | (250) |
| 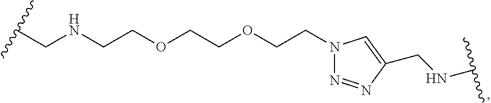 [SMILES: *CNCCOCCOCCn1cc(CN*)nn1] | (251) |
| 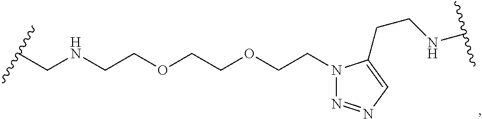 [SMILES: *CNCCOCCOCCn1nncc1CCN*] | (253) |
| 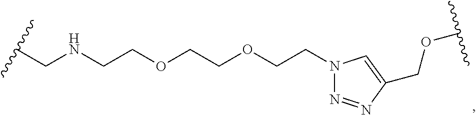 [SMILES: *CNCCOCCOCCn1cc(CO*)nn1] | (254) |
| 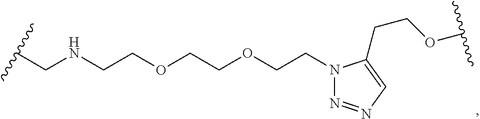 [SMILES: *CNCCOCCOCCn1nncc1CCO*] | (255) |
| 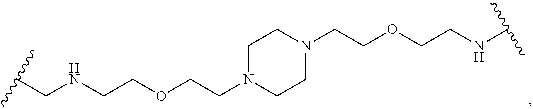 [SMILES: *CNCCOCCN1CCN(CCOCCN*)CC1] | (256) |
| 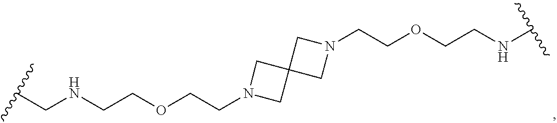 [SMILES: *CNCCOCCN1CC2(CN(CCOCCN*)C2)C1] | (257) |
| 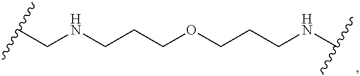 [SMILES: *CNCCCOCCCN*] | (258) |
| 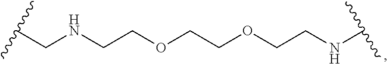 [SMILES: *CNCCOCCOCCN*] | (259) |
| 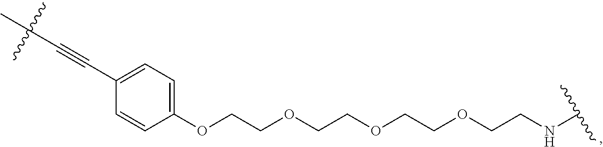 [SMILES: *C#Cc1ccc(OCCOCCOCCOCCN*)cc1] | (260) |
| 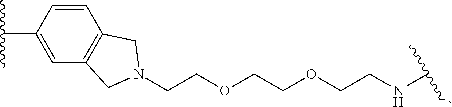 [SMILES: *NCCOCCOCCN1Cc2ccc(*)cc2C1] | (261) |
| 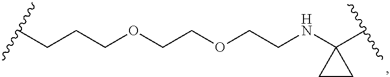 [SMILES: *CCCOCCOCCNC1(*)CC1] | (262) |
| 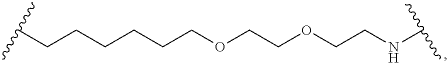 [SMILES: *CCCCCCOCCOCCN*] | (263) |
| 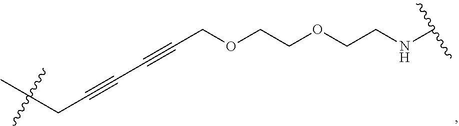 [SMILES: *CC#CC#CCOCCOCCN*] | (264) |
| 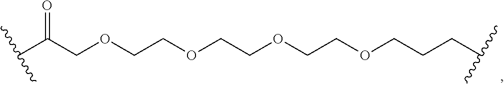 [SMILES: *CCCOCCOCCOCCOCC(*)=O] | (265) |
| 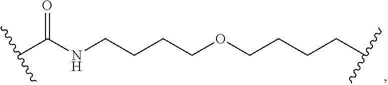 [SMILES: *CCCCOCCCCNC(*)=O] | (266) |
| 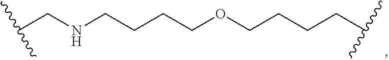 [SMILES: *CCCCOCCCCNC*] | (267) |
| 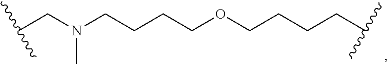 [SMILES: *CCCCOCCCCN(C)C*] | (268) |
| 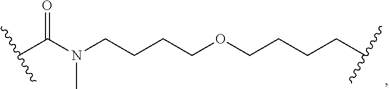 [SMILES: *CCCCOCCCCN(C)C(*)=O] | (269) |
| 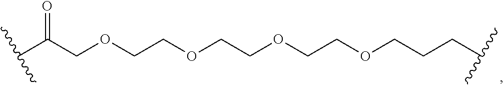 [SMILES: *CCCOCCOCCOCCOCC(*)=O] | (270) |
| 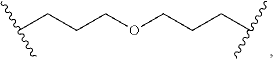 [SMILES: *CCCOCCC*] | (271) |
| 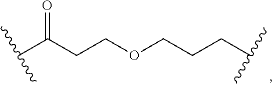 [SMILES: *CCCOCCC(*)=O] | (272) |
| 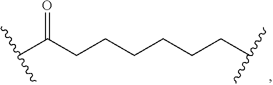 [SMILES: *CCCCCCC(*)=O] | (273) |
| 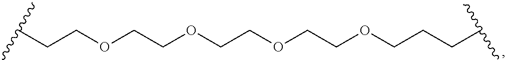 [SMILES: *CCCOCCOCCOCCOCC*] | (274) |
| 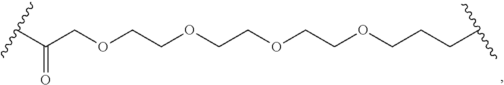 [SMILES: *CCCOCCOCCOCCOCC(*)=O] | (275) |
| 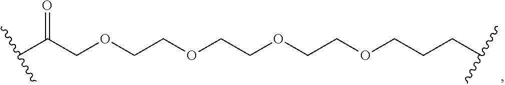 [SMILES: *CCCOCCOCCOCCOCC(*)=O] | (276) |
| 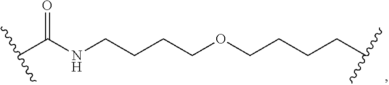 [SMILES: *CCCCOCCCCNC(*)=O] | (277) |
| 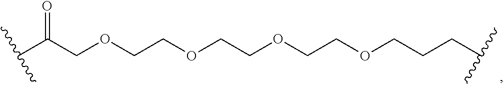 [SMILES: *CCCOCCOCCOCCOCC(*)=O] | (278) |
| 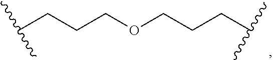 [SMILES: *CCCOCCC*] | (279) |
| 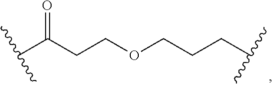 [SMILES: *CCCOCCC(*)=O] | (280) |
| 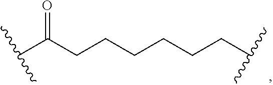 [SMILES: *CCCCCCC(*)=O] | (281) |
| 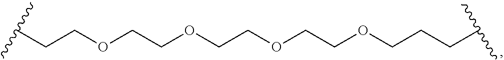 [SMILES: *CCCOCCOCCOCCOCC*] | (282) |
| 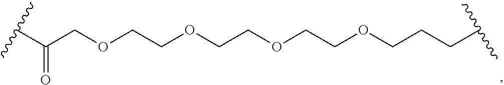 [SMILES: *CCCOCCOCCOCCOCC(*)=O] | (283) |
| 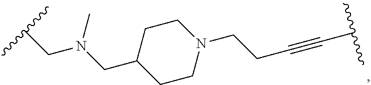 [SMILES: *C#CCCN1CCC(CN(C)C*)CC1] | (284) |
| 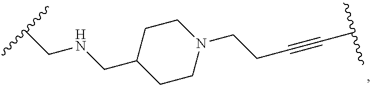 [SMILES: *C#CCCN1CCC(CNC*)CC1] | (285) |
| 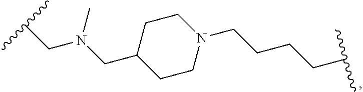 [SMILES: *CCCCN1CCC(CN(C)C*)CC1] | (286) |
| 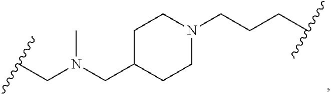 [SMILES: *CCCN1CCC(CN(C)C*)CC1] | (287) |
| 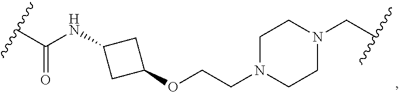 [SMILES: *CN1CCN(CCO[C@H]2C[C@H](NC(*)=O)C2)CC1] | (288) |
| 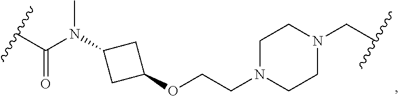 [SMILES: *CN1CCN(CCO[C@H]2C[C@H](N(C)C(*)=O)C2)CC1] | (289) |
| 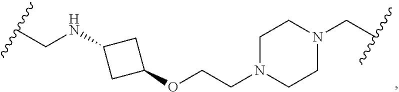 [SMILES: *CN[C@H]1C[C@H](OCCN2CCN(C*)CC2)C1] | (290) |
| 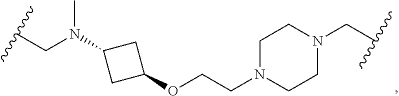 [SMILES: *CN1CCN(CCO[C@H]2C[C@H](N(C)C*)C2)CC1] | (291) |
| 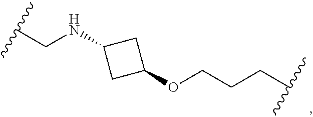 [SMILES: *CCCO[C@H]1C[C@H](NC*)C1] | (292) |
| 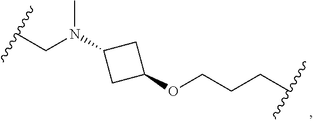 [SMILES: *CCCO[C@H]1C[C@H](N(C)C*)C1] | (293) |
| 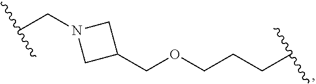 [SMILES: *CCCOCC1CN(C*)C1] | (294) |
| 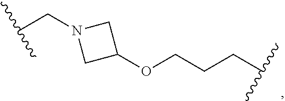 [SMILES: *CCCOC1CN(C*)C1] | (295) |
| 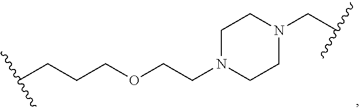 [SMILES: *CCCOCCN1CCN(C*)CC1] | (296) |
| 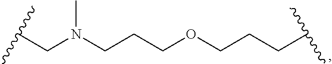 [SMILES: *CCCOCCCN(C)C*] | (297) |
| 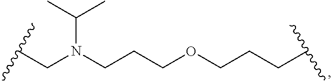 [SMILES: *CCCOCCCN(C*)C(C)C] | (298) |
| 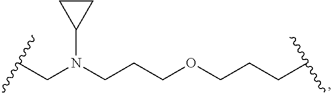 [SMILES: *CCCOCCCN(C*)C1CC1] | (299) |
| 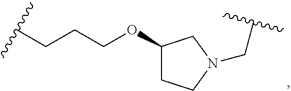 [SMILES: *CCCO[C@@H]1CCN(C*)C1] | (300) |
| 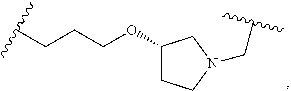 [SMILES: *CCCO[C@H]1CCN(C*)C1] | (301) |
| 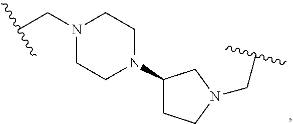 [SMILES: *CN1CCN([C@@H]2CCN(C*)C2)CC1] | (302) |
| 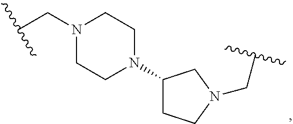 [SMILES: *CN1CCN([C@H]2CCN(C*)C2)CC1] | (303) |
| 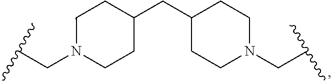 [SMILES: *CN1CCC(CC2CCN(C*)CC2)CC1] | (304) |
| 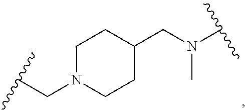 [SMILES: *CN1CCC(CN(*)C)CC1] | (305) |
| 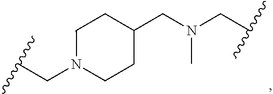 [SMILES: *CN(C)CC1CCN(C*)CC1] | (306) |
| 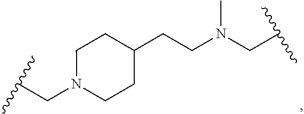 [SMILES: *CN(C)CCC1CCN(C*)CC1] | (307) |
| 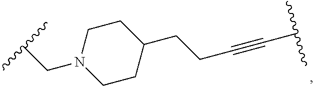 [SMILES: *C#CCCC1CCN(C*)CC1] | (308) |
| 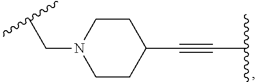 [SMILES: *C#CC1CCN(C*)CC1] | (309) |
| 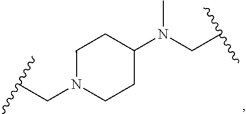 [SMILES: *CN1CCC(N(C)C*)CC1] | (310) |
| 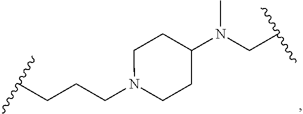 [SMILES: *CCCN1CCC(N(C)C*)CC1] | (311) |
| 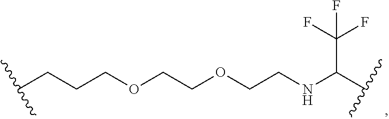 [SMILES: *CCCOCCOCCNC(*)C(F)(F)F] | (312) |
| 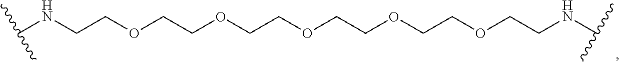 [SMILES: *NCCOCCOCCOCCOCCOCCN*] | (313) |
| 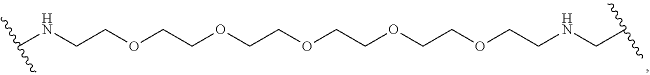 [SMILES: *CNCCOCCOCCOCCOCCOCCN*] | (314) |
| 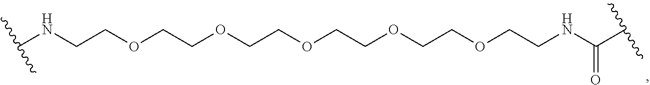 [SMILES: *NCCOCCOCCOCCOCCOCCNC(*)=O] | (315) |
| 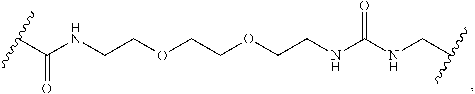 [SMILES: *CNC(=O)NCCOCCOCCNC(*)=O] | (316) |
| 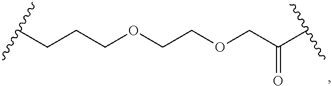 [SMILES: *CCCOCCOCC(*)=O] | (317) |
| 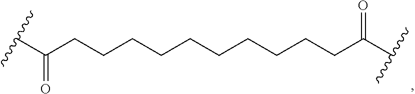 [SMILES: *C(=O)CCCCCCCCCCC(*)=O] | (318) |
| 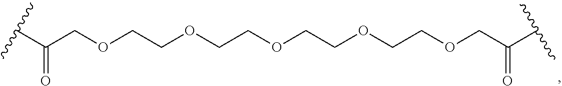 [SMILES: *C(=O)COCCOCCOCCOCCOCC(*)=O] | (319) |
| 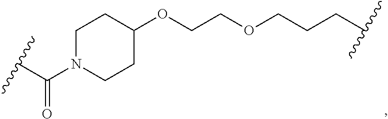 [SMILES: *CCCOCCOC1CCN(C(*)=O)CC1] | (320) |
| 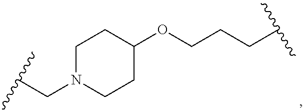 [SMILES: *CCCOC1CCN(C*)CC1] | (321) |
| 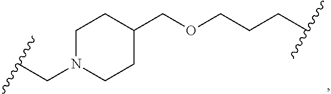 [SMILES: *CCCOCC1CCN(C*)CC1] | (322) |
| 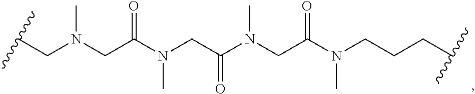 [SMILES: *CCCN(C)C(=O)CN(C)C(=O)CN(C)C(=O)CN(C)C*] | (323) |
| 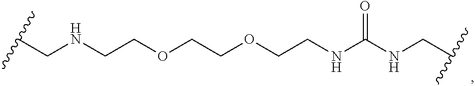 [SMILES: *CNCCOCCOCCNC(=O)NC*] | (324) |
| 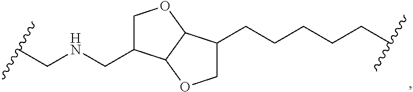 [SMILES: *CCCCCC1COC2C(CNC*)COC12] | (325) |
| 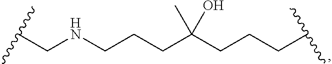 [SMILES: *CCCC(C)(O)CCCNC*] | (326) |
| 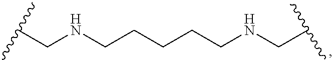 [SMILES: *CNCCCCCNC*] | (327) |
| 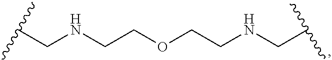 [SMILES: *CNCCOCCNC*] | (328) |
| 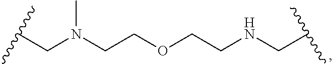 [SMILES: *CNCCOCCN(C)C*] | (329) |
| 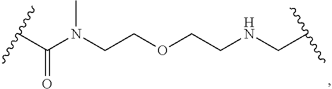 [SMILES: *CNCCOCCN(C)C(*)=O] | (330) |
| 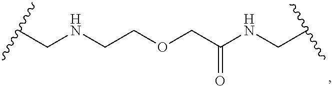 [SMILES: *CNCCOCC(=O)NC*] | (331) |
| 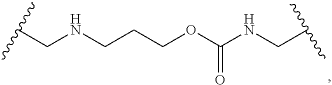 [SMILES: *CNCCCOC(=O)NC*] | (332) |
| 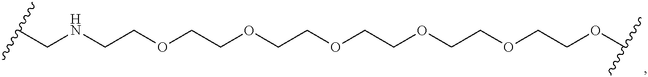 [SMILES: *CNCCOCCOCCOCCOCCOCCO*] | (333) |
| 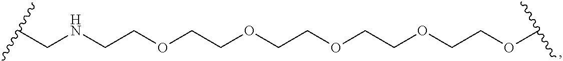 [SMILES: *CNCCOCCOCCOCCOCCO*] | (334) |
| 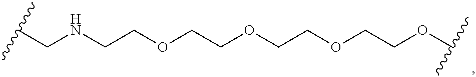 [SMILES: *CNCCOCCOCCOCCO*] | (335) |
| 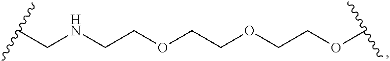 [SMILES: *CNCCOCCOCCO*] | (336) |
| 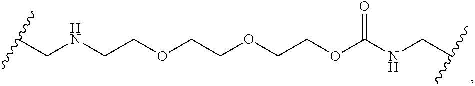 [SMILES: *CNCCOCCOCCOC(=O)NC*] | (337) |
| 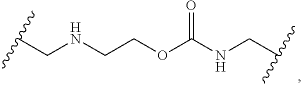 [SMILES: *CNCCOC(=O)NC*] | (338) |
| 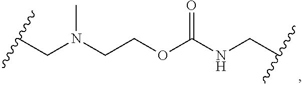 [SMILES: *CNC(=O)OCCN(C)C*] | (339) |
| 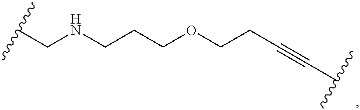 [SMILES: *C#CCCOCCCNC*] | (340) |
| 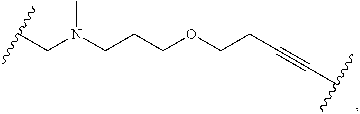 [SMILES: *C#CCCOCCCN(C)C*] | (341) |
| 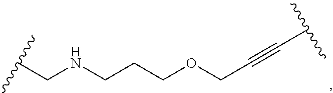 [SMILES: *C#CCOCCCNC*] | (342) |
| 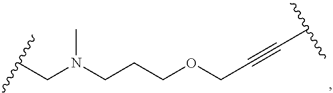 [SMILES: *C#CCOCCCN(C)C*] | (343) |
| 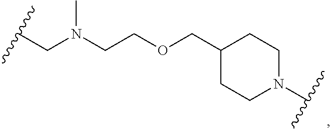 [SMILES: *CN(C)CCOCC1CCN(*)CC1] | (344) |
| 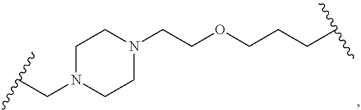 [SMILES: *CCCOCCN1CCN(C*)CC1] | (345) |
| 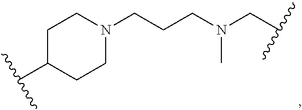 [SMILES: *CN(C)CCCN1CCC(*)CC1] | (346) |
| 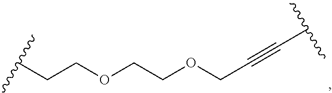 [SMILES: *C#CCOCCOCC*] | (347) |
| 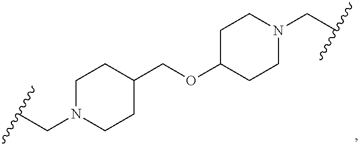 [SMILES: *CN1CCC(COC2CCN(C*)CC2)CC1] | (348) |
| 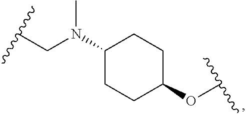 [SMILES: *CN(C)[C@H]1CC[C@H](O*)CC1] | (349) |
| 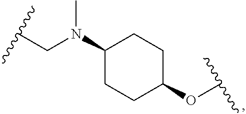 [SMILES: *CN(C)[C@H]1CC[C@@H](O*)CC1] | (350) |
| 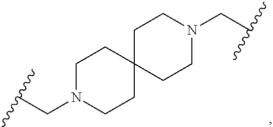 [SMILES: *CN1CCC2(CC1)CCN(C*)CC2] | (351) |
| 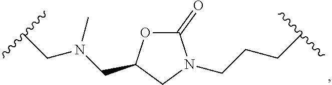 [SMILES: *CCCN1C[C@@H](CN(C)C*)OC1=O] | (352) |
| 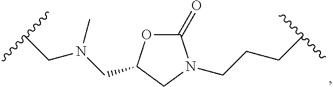 [SMILES: *CCCN1C[C@H](CN(C)C*)OC1=O] | (353) |
| 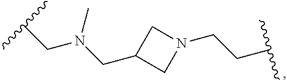 [SMILES: *CCN1CC(CN(C)C*)C1] | (354) |
| 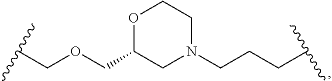 [SMILES: *CCCN1CCO[C@@H](COC*)C1] | (355) |
| 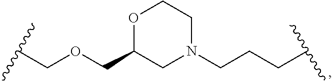 [SMILES: *CCCN1CCO[C@H](COC*)C1] | (356) |
| 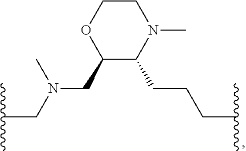 [SMILES: *CCC[C@@H]1[C@@H](CN(C)C*)OCCN1C] | (357) |
| 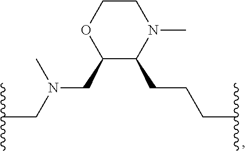 [SMILES: *CCC[C@H]1[C@@H](CN(C)C*)OCCN1C] | (358) |
| 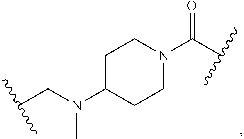 [SMILES: *CN(C)C1CCN(C(*)=O)CC1] | (359) |
| 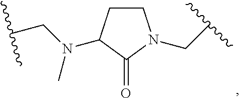 [SMILES: *CN1CCC(N(C)C*)C1=O] | (360) |
| 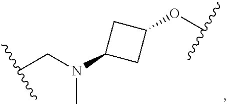 [SMILES: *CN(C)[C@H]1C[C@H](O*)C1] | (361) |
| 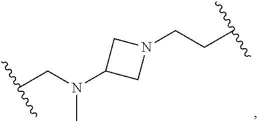 [SMILES: *CCN1CC(N(C)C*)C1] | (362) |
| 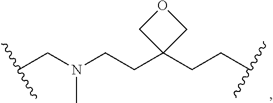 [SMILES: *CCC1(CCN(C)C*)COC1] | (363) |
| 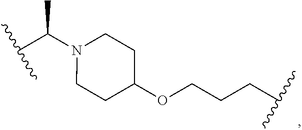 [SMILES: *CCCOC1CCN([C@@H](*)C)CC1] | (364) |
| 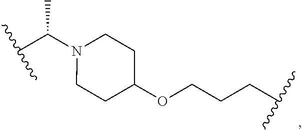 [SMILES: *CCCOC1CCN([C@H](*)C)CC1] | (365) |
| 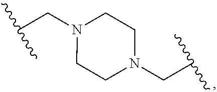 [SMILES: *CN1CCN(C*)CC1] | (366) |
| 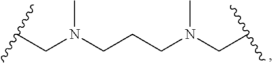 [SMILES: *CN(C)CCCN(C)C*] | (367) |
| 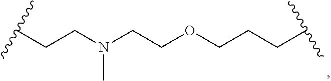 [SMILES: *CCCOCCN(C)CC*] | (368) |
| 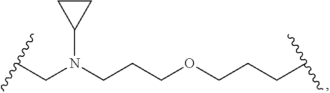 [SMILES: *CCCOCCCN(C*)C1CC1] | (369) |
| 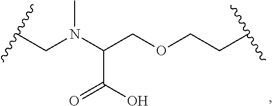 [SMILES: *CCOCC(C(=O)O)N(C)C*] | (370) |
| 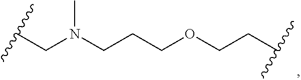 [SMILES: *CCOCCCN(C)C*] | (371) |
| 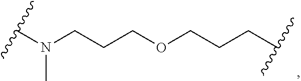 [SMILES: *CCCOCCCN(*)C] | (372) |
| 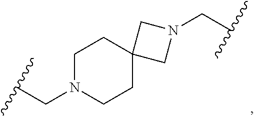 [SMILES: *CN1CCC2(CC1)CN(C*)C2] | (373) |
| 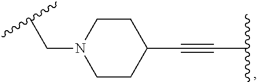 [SMILES: *C#CC1CCN(C*)CC1] | (374) |
| 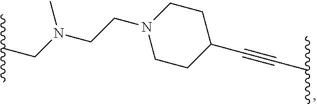 [SMILES: *C#CC1CCN(CCN(C)C*)CC1] | (375) |
| 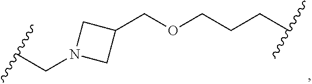 [SMILES: *CCCOCC1CN(C*)C1] | (376) |
| 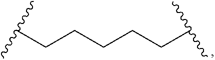 [SMILES: *CCCCC*] | (377) |
| 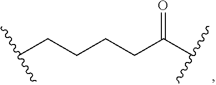 [SMILES: *CCCCC(*)=O] | (378) |
| 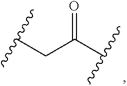 [SMILES: *CC(*)=O] | (379) |
| 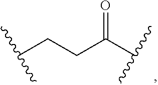 [SMILES: *CCC(*)=O] | (380) |
| 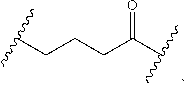 [SMILES: *CCCC(*)=O] | (381) |
| 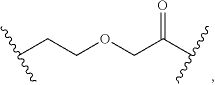 [SMILES: *CCOCC(*)=O] | (382) |
| 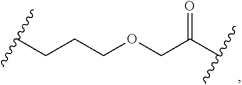 [SMILES: *CCCOCC(*)=O] | (383) |
| 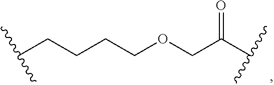 [SMILES: *CCCCOCC(*)=O] | (384) |
| 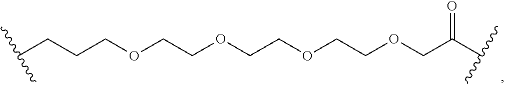 [SMILES: *CCCOCCOCCOCCOCC(*)=O] | (385) |
| 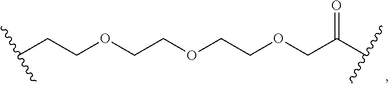 [SMILES: *CCOCCOCCOCC(*)=O] | (386) |
| 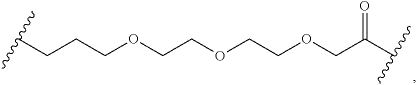 [SMILES: *CCCOCCOCCOCC(*)=O] | (387) |
| 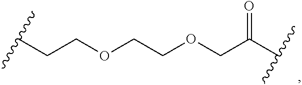 [SMILES: *CCOCCOCC(*)=O] | (388) |
| 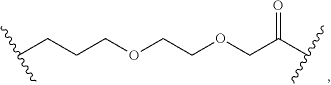 [SMILES: *CCCOCCOCC(*)=O] | (389) |
| 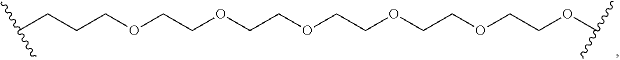 [SMILES: *CCCOCCOCCOCCOCCOCCO*] | (390) |
| 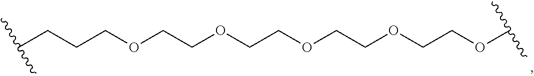 [SMILES: *CCCOCCOCCOCCOCCO*] | (391) |
| 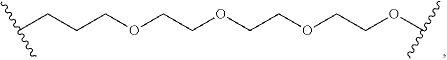 [SMILES: *CCCOCCOCCOCCO*] | (392) |
| 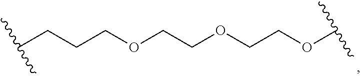 [SMILES: *CCCOCCOCCO*] | (393) |
| 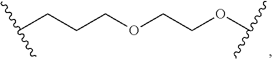 [SMILES: *CCCOCCO*] | (394) |
| 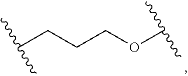 [SMILES: *CCCO*] | (395) |
| 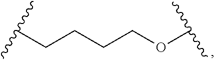 [SMILES: *CCCCO*] | (396) |
| 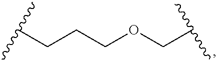 [SMILES: *CCCOC*] | (397) |
| 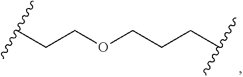 [SMILES: *CCCOCC*] | (398) |
| 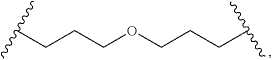 [SMILES: *CCCOCCC*] | (399) |
| 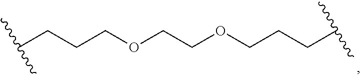 [SMILES: *CCCOCCOCCC*] | (400) |
| 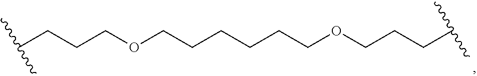 [SMILES: *CCCOCCCCCCOCCC*] | (401) |
| 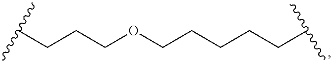 [SMILES: *CCCCCOCCC*] | (402) |
| 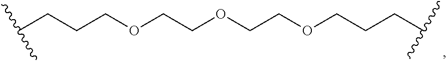 [SMILES: *CCCOCCOCCOCCC*] | (403) |
| 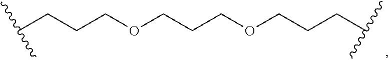 [SMILES: *CCCOCCCOCCC*] | (404) |
| 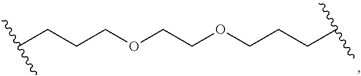 [SMILES: *CCCOCCOCCC*] | (405) |
| 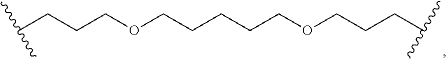 [SMILES: *CCCOCCCCCOCCC*] | (406) |
| 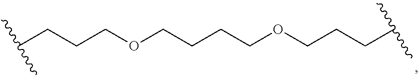 [SMILES: *CCCOCCCCOCCC*] | (407) |
| 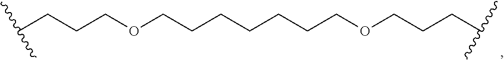 [SMILES: *CCCOCCCCCCCOCCC*] | (408) |
| 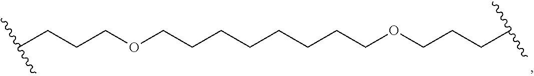 [SMILES: *CCCOCCCCCCCCOCCC*] | (409) |
| 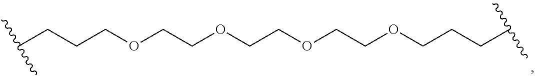 [SMILES: *CCCOCCOCCOCCOCCC*] | (410) |
| 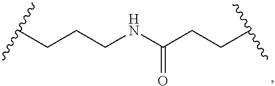 [SMILES: *CCCNC(=O)CC*] | (411) |
| 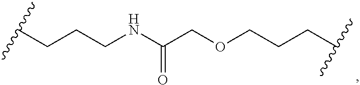 [SMILES: *CCCNC(=O)COCCC*] | (412) |
| 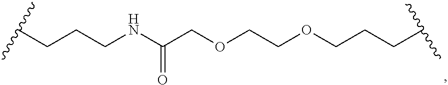 [SMILES: *CCCNC(=O)COCCOCCC*] | (413) |
| 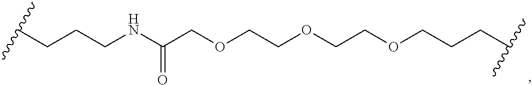 [SMILES: *CCCNC(=O)COCCOCCOCCC*] | (414) |
| 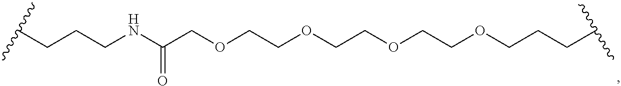 [SMILES: *CCCNC(=O)COCCOCCOCCOCCC*] | (415) |
| 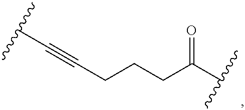 [SMILES: *C#CCCCC(*)=O] | (416) |
| 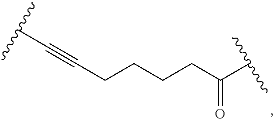 [SMILES: *C#CCCCCC(*)=O] | (417) |
| 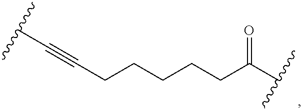 [SMILES: *C#CCCCCCC(*)=O] | (418) |
| 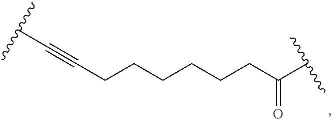 [SMILES: *C#CCCCCCCC(*)=O] | (419) |
| 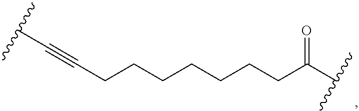 [SMILES: *C#CCCCCCCCC(*)=O] | (420) |
| 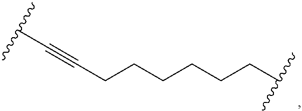 [SMILES: *C#CCCCCCC*] | (421) |
| 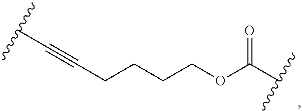 [SMILES: *C#CCCCCOC(*)=O] | (422) |
| 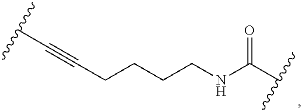 [SMILES: *C#CCCCCNC(*)=O] | (423) |
| 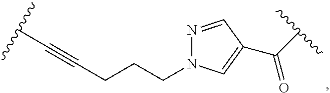 [SMILES: *C#CCCCn1cc(C(*)=O)cn1] | (424) |
| 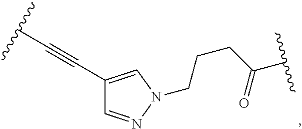 [SMILES: *C#Cc1cnn(CCCC(*)=O)c1] | (425) |
| 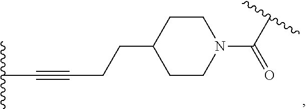 [SMILES: *C#CCCC1CCN(C(*)=O)CC1] | (426) |
| 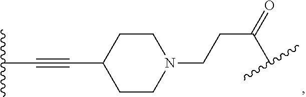 [SMILES: *C#CC1CCN(CCC(*)=O)CC1] | (427) |
| 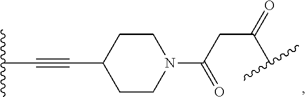 [SMILES: *C#CC1CCN(C(=O)CC(*)=O)CC1] | (428) |
| 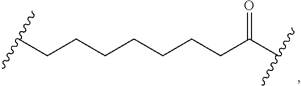 [SMILES: *CCCCCCCC(*)=O] | (429) |
| 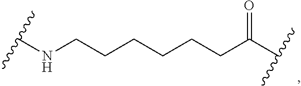 [SMILES: *NCCCCCCC(*)=O] | (430) |
| 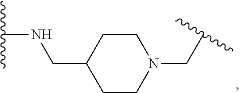 [SMILES: *CN1CCC(CN*)CC1] | (431) |
| 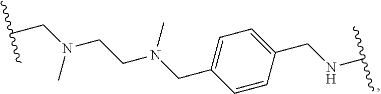 [SMILES: *CN(C)CCN(C)Cc1ccc(CN*)cc1] | (432) |
| 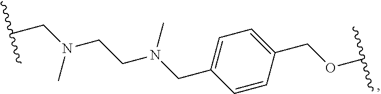 [SMILES: *CN(C)CCN(C)Cc1ccc(CO*)cc1] | (433) |
| 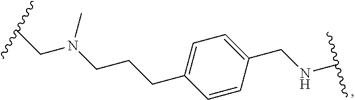 [SMILES: *CN(C)CCCc1ccc(CN*)cc1] | (434) |
| 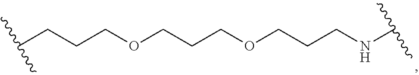 [SMILES: *CCCOCCCOCCCN*] | (435) |
| 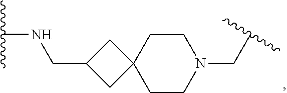 [SMILES: *CN1CCC2(CC1)CC(CN*)C2] | (436) |
| 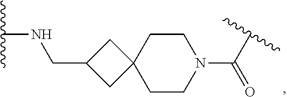 [SMILES: *NCC1CC2(CCN(C(*)=O)CC2)C1] | (437) |
| 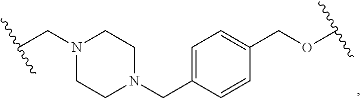 [SMILES: *CN1CCN(Cc2ccc(CO*)cc2)CC1] | (438) |
| 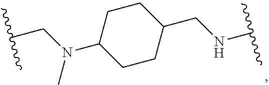 [SMILES: *CN(C)C1CCC(CN*)CC1] | (438) |
| 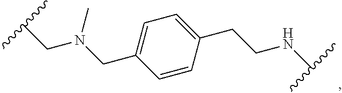 [SMILES: *CN(C)Cc1ccc(CCN*)cc1] | (439) |
| 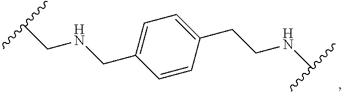 [SMILES: *CNCc1ccc(CCN*)cc1] | (440) |
| 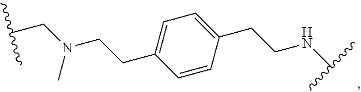 [SMILES: *CN(C)CCc1ccc(CCN*)cc1] | (441) |
| 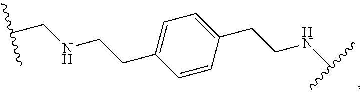 [SMILES: *CNCCc1ccc(CCN*)cc1] | (442) |
| 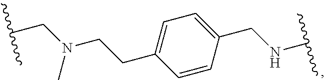 [SMILES: *CN(C)CCc1ccc(CN*)cc1] | (443) |
| 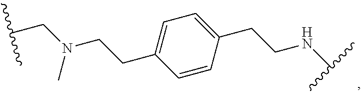 [SMILES: *CN(C)CCc1ccc(CCN*)cc1] | (444) |
| 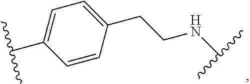 [SMILES: *NCCc1ccc(*)cc1] | (445) |
| 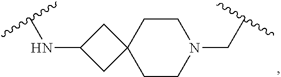 [SMILES: *CN1CCC2(CC1)CC(N*)C2] | (446) |
| 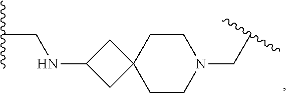 [SMILES: *CNC1CC2(CCN(C*)CC2)C1] | (447) |
| 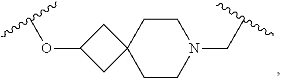 [SMILES: *CN1CCC2(CC1)CC(O*)C2] | (448) |
| 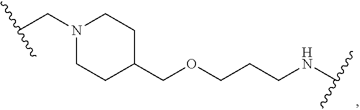 [SMILES: *CN1CCC(COCCCN*)CC1] | (449) |
| 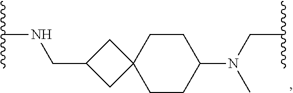 [SMILES: *CN(C)C1CCC2(CC1)CC(CN*)C2] | (450) |
| 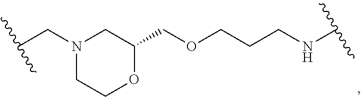 [SMILES: *CN1CCO[C@@H](COCCCN*)C1] | (451) |
| 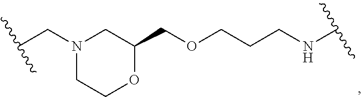 [SMILES: *CN1CCO[C@H](COCCCN*)C1] | (452) |
| 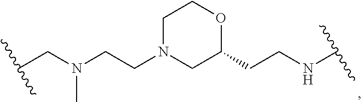 [SMILES: *CN(C)CCN1CCO[C@H](CCN*)C1] | (453) |
| 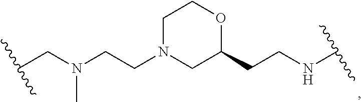 [SMILES: *CN(C)CCN1CCO[C@@H](CCN*)C1] | (454) |
| 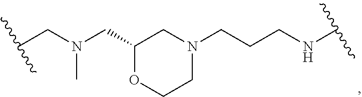 [SMILES: *CN(C)C[C@@H]1CN(CCCN*)CCO1] | (455) |
| 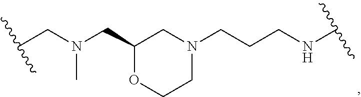 [SMILES: *CN(C)C[C@H]1CN(CCCN*)CCO1] | (456) |
| 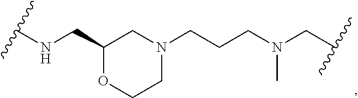 [SMILES: *CN(C)CCCN1CCO[C@@H](CN*)C1] | (457) |
| 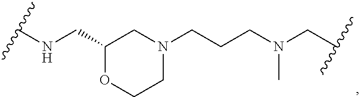 [SMILES: *CN(C)CCCN1CCO[C@H](CN*)C1] | (458) |
| 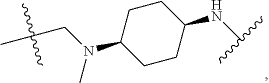 [SMILES: *CN(C)[C@H]1CC[C@@H](N*)CC1] | (459) |
| 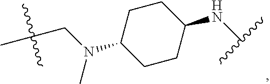 [SMILES: *CN(C)[C@H]1CC[C@H](N*)CC1] | (460) |
| 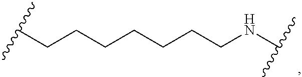 [SMILES: *CCCCCCCN*] | (461) |
| 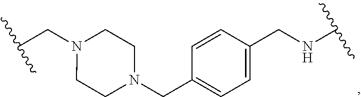 [SMILES: *CN1CCN(Cc2ccc(CN*)cc2)CC1] | (462) |
| 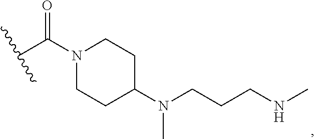 [SMILES: *C(=O)N1CCC(N(C)CCCNC)CC1] | (463) |
| 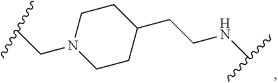 [SMILES: *CN1CCC(CCN*)CC1] | (464) |
| 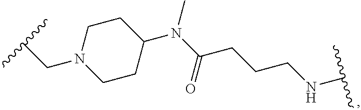 [SMILES: *CN1CCC(N(C)C(=O)CCCN*)CC1] | (465) |
| 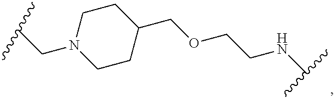 [SMILES: *CN1CCC(COCCN*)CC1] | (466) |
| 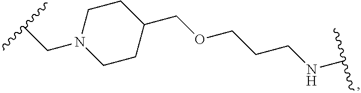 [SMILES: *CN1CCC(COCCCN*)CC1] | (467) |
| 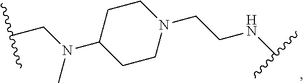 [SMILES: *CN(C)C1CCN(CCN*)CC1] | (468) |
| 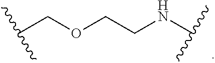 [SMILES: *COCCN*] | (469) |
| 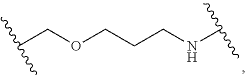 [SMILES: *COCCCN*] | (470) |
| 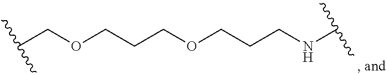 [SMILES: *COCCCOCCCN*] | (471) |
| 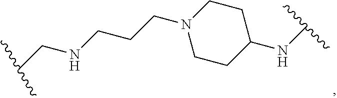 [SMILES: *CNCCCN1CCC(N*)CC1] | (472) |

In some embodiments, the present invention provides a compound having a STAT3 binding moiety described and disclosed herein, a LBM set forth in Table A above, and a linker set forth in Table B above, or a pharmaceutically acceptable salt thereof

Exemplary compounds of the invention are set forth in Table 1, below.

TABLE 1
Exemplary Compounds

| I-# | Structure |
|---|---|
| I-1 | 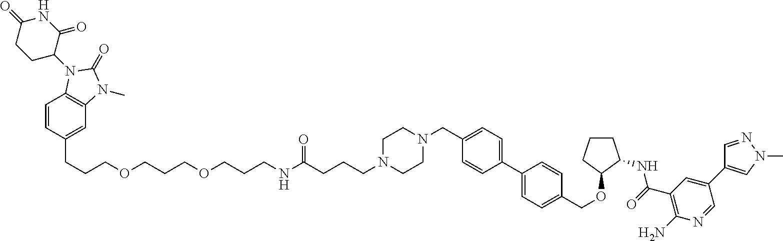 [SMILES: Cn1cc(-c2cnc(N)c(C(=O)N[C@H]3CCC[C@@H]3OCc3ccc(-c4ccc(CN5CCN(CCCC(=O)NCCCOCCCOCCCc6ccc7c(c6)n(C)c(=O)n7C6CCC(=O)NC6=O)CC5)cc4)cc3)c2)cn1] |
| I-2 | 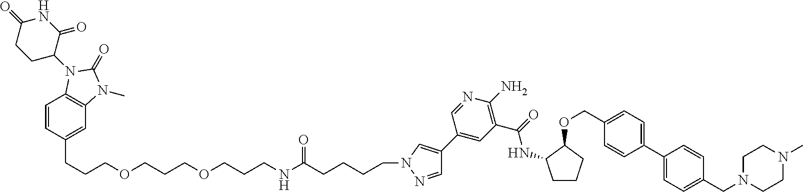 [SMILES: CN1CCN(Cc2ccc(-c3ccc(CO[C@H]4CCC[C@@H]4NC(=O)c4cc(-c5cnn(CCCCC(=O)NCCCOCCCOCCCc6ccc7c(c6)n(C)c(=O)n7C6CCC(=O)NC6=O)c5)cnc4N)cc3)cc2)CC1] |
| I-3 | 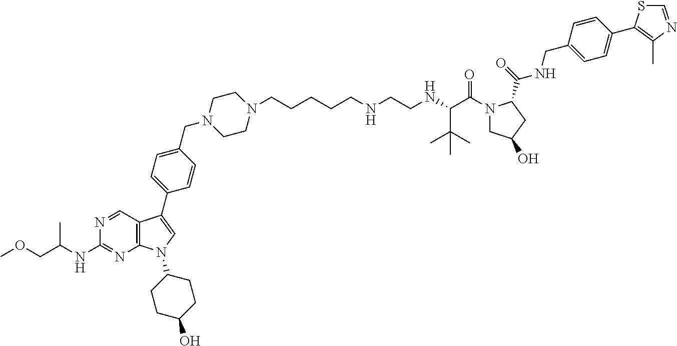 |
| I-4 | 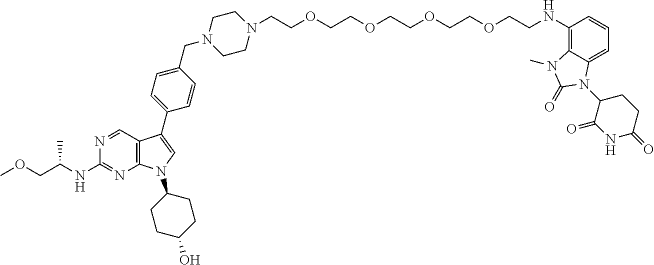 [SMILES: COC[C@H](C)Nc1ncc2c(-c3ccc(CN4CCN(CCOCCOCCOCCOCCNc5cccc6c5n(C)c(=O)n6C5CCC(=O)NC5=O)CC4)cc3)cn([C@H]3CC[C@H](O)CC3)c2n1] |
| I-5 | 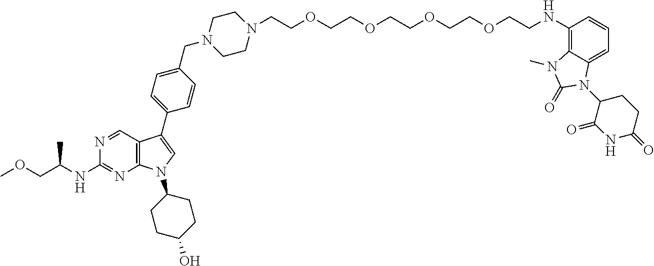 [SMILES: COC[C@@H](C)Nc1ncc2c(-c3ccc(CN4CCN(CCOCCOCCOCCOCCNc5cccc6c5n(C)c(=O)n6C5CCC(=O)NC5=O)CC4)cc3)cn([C@H]3CC[C@H](O)CC3)c2n1] |
| I-6 | 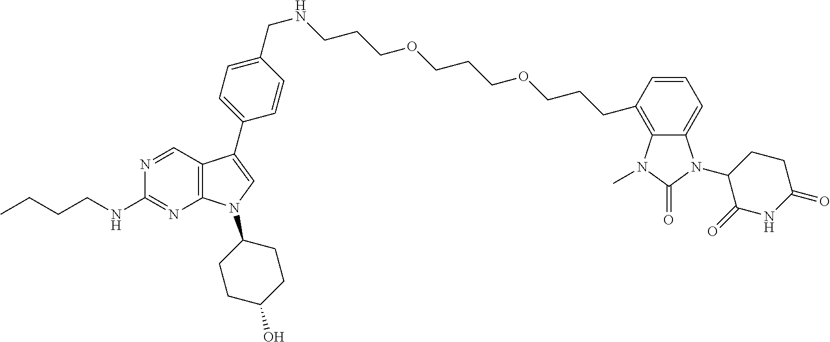 [SMILES: CCCCNc1ncc2c(-c3ccc(CNCCCOCCCOCCCc4cccc5c4n(C)c(=O)n5C4CCC(=O)NC4=O)cc3)cn([C@H]3CC[C@H](O)CC3)c2n1] |
| I-7 | 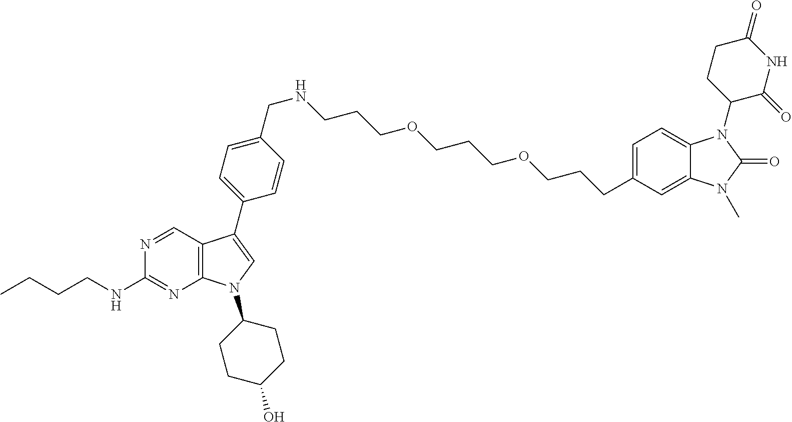 [SMILES: CCCCNc1ncc2c(-c3ccc(CNCCCOCCCOCCCc4ccc5c(c4)n(C)c(=O)n5C4CCC(=O)NC4=O)cc3)cn([C@H]3CC[C@H](O)CC3)c2n1] |
| I-8 | 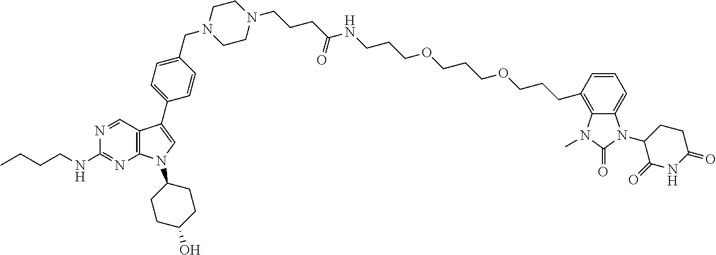 [SMILES: CCCCNc1ncc2c(-c3ccc(CN4CCN(CCCC(=O)NCCCOCCCOCCCc5cccc6c5n(C)c(=O)n6C5CCC(=O)NC5=O)CC4)cc3)cn([C@H]3CC[C@H](O)CC3)c2n1] |
| I-9 | 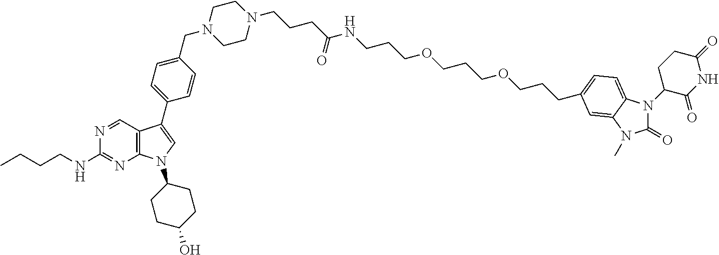 [SMILES: CCCCNc1ncc2c(-c3ccc(CN4CCN(CCCC(=O)NCCCOCCCOCCCc5ccc6c(c5)n(C)c(=O)n6C5CCC(=O)NC5=O)CC4)cc3)cn([C@H]3CC[C@H](O)CC3)c2n1] |
| I-10 | 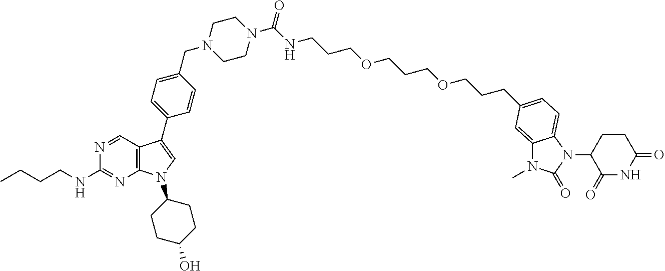 [SMILES: CCCCNc1ncc2c(-c3ccc(CN4CCN(C(=O)NCCCOCCCOCCCc5ccc6c(c5)n(C)c(=O)n6C5CCC(=O)NC5=O)CC4)cc3)cn([C@H]3CC[C@H](O)CC3)c2n1] |
| I-11 | 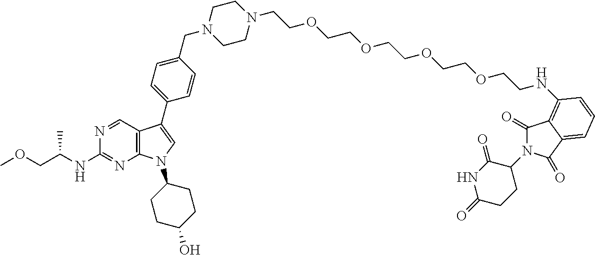 [SMILES: COC[C@H](C)Nc1ncc2c(-c3ccc(CN4CCN(CCOCCOCCOCCOCCNc5cccc6c5C(=O)N(C5CCC(=O)NC5=O)C6=O)CC4)cc3)cn([C@H]3CC[C@H](O)CC3)c2n1] |
| I-12 | 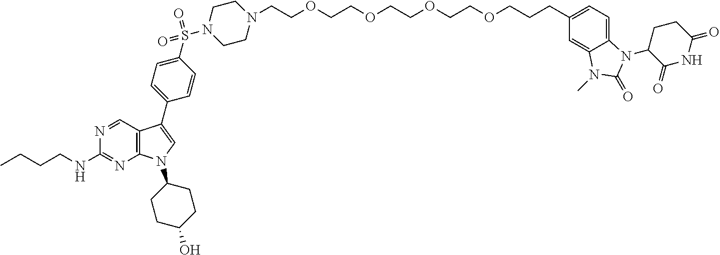 [SMILES: CCCCNc1ncc2c(-c3ccc(S(=O)(=O)N4CCN(CCOCCOCCOCCOCCCc5ccc6c(c5)n(C)c(=O)n6C5CCC(=O)NC5=O)CC4)cc3)cn([C@H]3CC[C@H](O)CC3)c2n1] |
| I-13 | 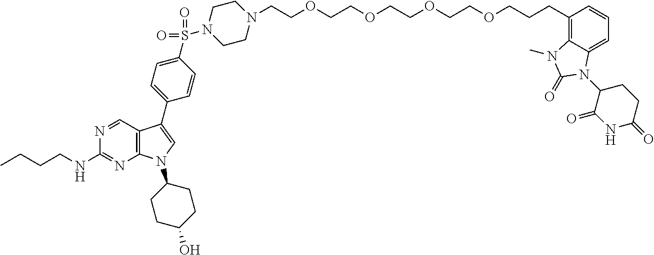 [SMILES: CCCCNc1ncc2c(-c3ccc(S(=O)(=O)N4CCN(CCOCCOCCOCCOCCCc5cccc6c5n(C)c(=O)n6C5CCC(=O)NC5=O)CC4)cc3)cn([C@H]3CC[C@H](O)CC3)c2n1] |
| I-14 | 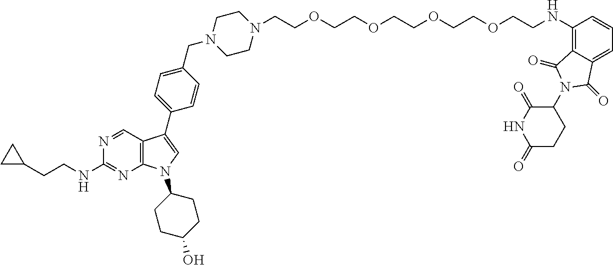 [SMILES: O=C1CCC(N2C(=O)c3cccc(NCCOCCOCCOCCOCCN4CCN(Cc5ccc(-c6cn([C@H]7CC[C@H](O)CC7)c7nc(NCCC8CC8)ncc67)cc5)CC4)c3C2=O)C(=O)N1] |
| I-15 | 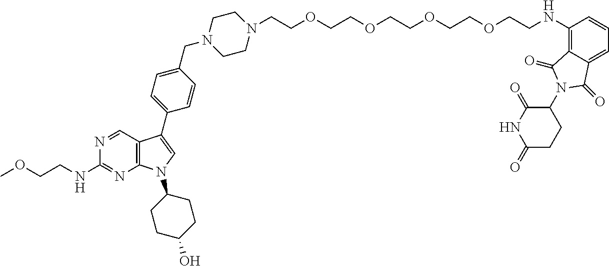 [SMILES: COCCNc1ncc2c(-c3ccc(CN4CCN(CCOCCOCCOCCOCCNc5cccc6c5C(=O)N(C5CCC(=O)NC5=O)C6=O)CC4)cc3)cn([C@H]3CC[C@H](O)CC3)c2n1] |
| I-19 | 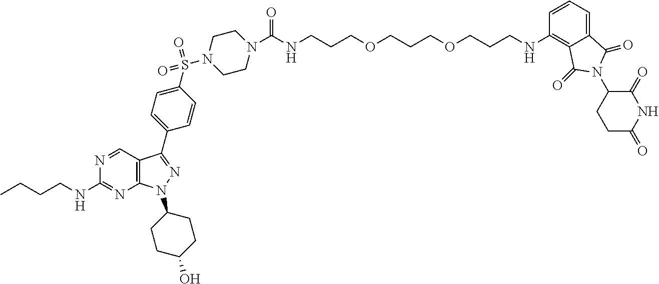 [SMILES: CCCCNc1ncc2c(-c3ccc(S(=O)(=O)N4CCN(C(=O)NCCCOCCCOCCCNc5cccc6c5C(=O)N(C5CCC(=O)NC5=O)C6=O)CC4)cc3)nn([C@H]3CC[C@H](O)CC3)c2n1] |
| I-20 | 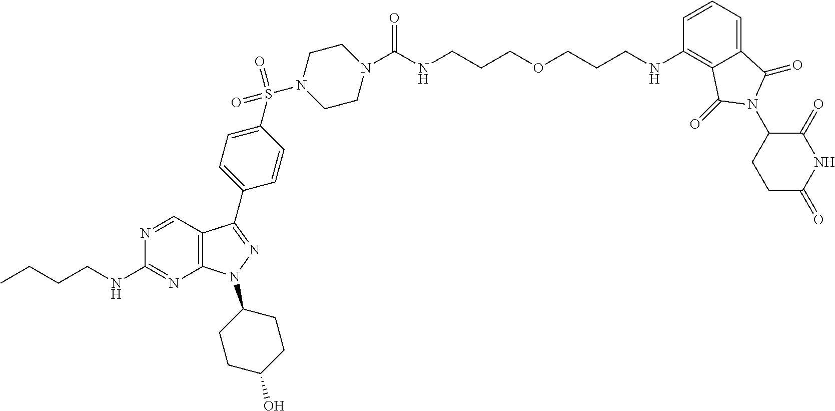 [SMILES: CCCCNc1ncc2c(-c3ccc(S(=O)(=O)N4CCN(C(=O)NCCCOCCCNc5cccc6c5C(=O)N(C5CCC(=O)NC5=O)C6=O)CC4)cc3)nn([C@H]3CC[C@H](O)CC3)c2n1] |
| I-21 | 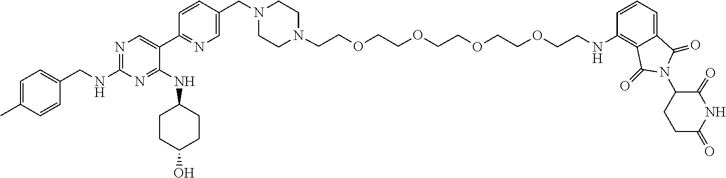 [SMILES: Cc1ccc(CNc2ncc(-c3ccc(CN4CCN(CCOCCOCCOCCOCCNc5cccc6c5C(=O)N(C5CCC(=O)NC5=O)C6=O)CC4)cn3)c(N[C@H]3CC[C@H](O)CC3)n2)cc1] |
| I-22 | 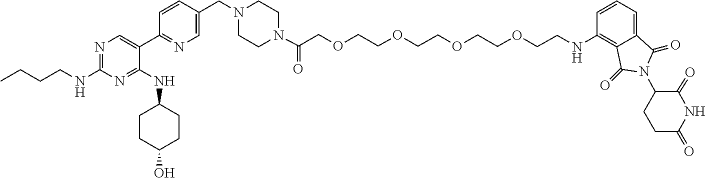 [SMILES: CCCCNc1ncc(-c2ccc(CN3CCN(C(=O)COCCOCCOCCOCCNc4cccc5c4C(=O)N(C4CCC(=O)NC4=O)C5=O)CC3)cn2)c(N[C@H]2CC[C@H](O)CC2)n1] |
| I-23 | 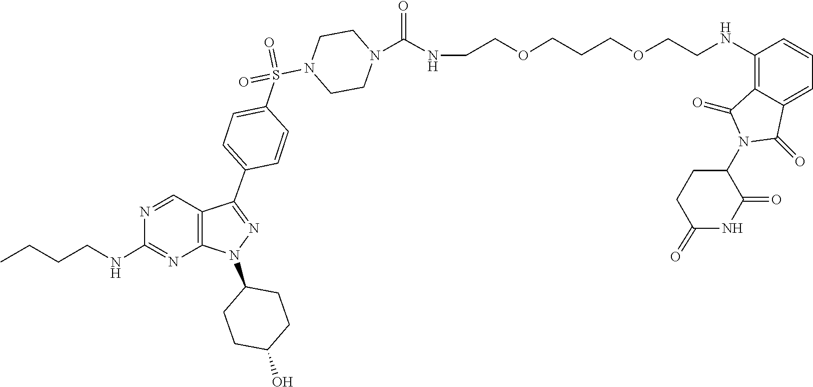 [SMILES: CCCCNc1ncc2c(-c3ccc(S(=O)(=O)N4CCN(C(=O)NCCOCCCOCCNc5cccc6c5C(=O)N(C5CCC(=O)NC5=O)C6=O)CC4)cc3)nn([C@H]3CC[C@H](O)CC3)c2n1] |
| I-24 | 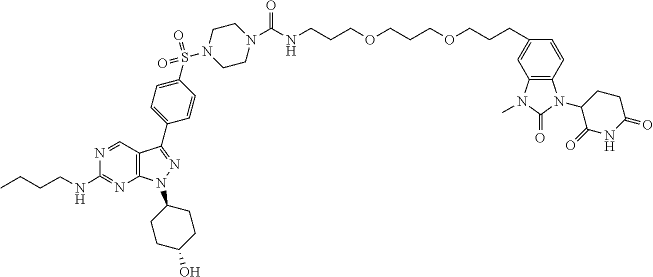 [SMILES: CCCCNc1ncc2c(-c3ccc(S(=O)(=O)N4CCN(C(=O)NCCCOCCCOCCCc5ccc6c(c5)n(C)c(=O)n6C5CCC(=O)NC5=O)CC4)cc3)nn([C@H]3CC[C@H](O)CC3)c2n1] |
| I-25 | 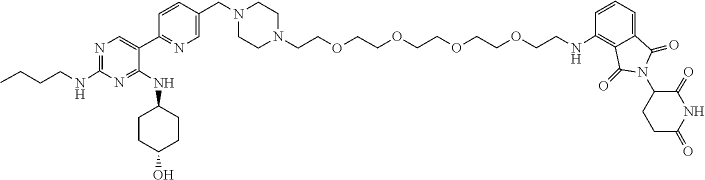 [SMILES: CCCCNc1ncc(-c2ccc(CN3CCN(CCOCCOCCOCCOCCNc4cccc5c4C(=O)N(C4CCC(=O)NC4=O)C5=O)CC3)cn2)c(N[C@H]2CC[C@H](O)CC2)n1] |
| I-26 | 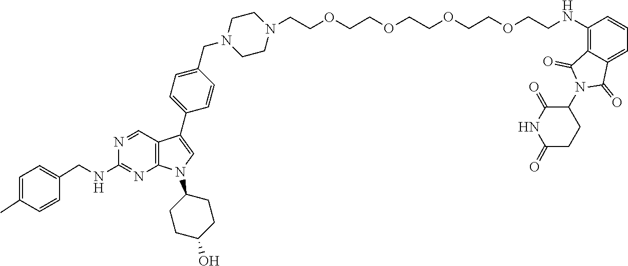 [SMILES: Cc1ccc(CNc2ncc3c(-c4ccc(CN5CCN(CCOCCOCCOCCOCCNc6cccc7c6C(=O)N(C6CCC(=O)NC6=O)C7=O)CC5)cc4)cn([C@H]4CC[C@H](O)CC4)c3n2)cc1] |
| I-27 | 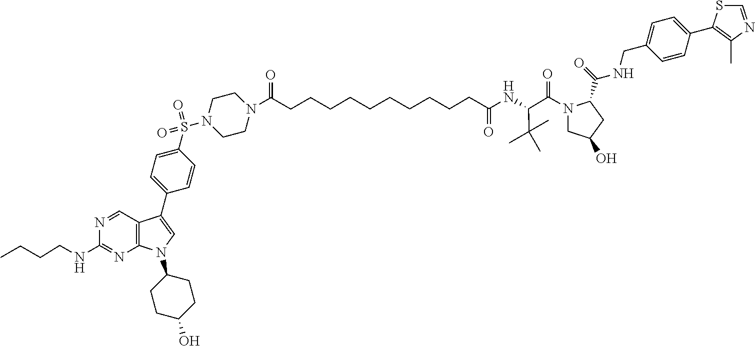 [SMILES: CCCCNc1ncc2c(-c3ccc(S(=O)(=O)N4CCN(C(=O)CCCCCCCCCCC(=O)N[C@H](C(=O)N5C[C@H](O)C[C@H]5C(=O)NCc5ccc(-c6scnc6C)cc5)C(C)(C)C)CC4)cc3)cn([C@H]3CC[C@H](O)CC3)c2n1] |
| I-28 | 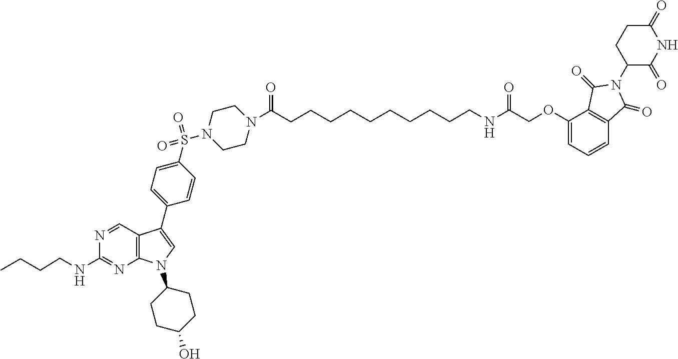 [SMILES: CCCCNc1ncc2c(-c3ccc(S(=O)(=O)N4CCN(C(=O)CCCCCCCCCCNC(=O)COc5cccc6c5C(=O)N(C5CCC(=O)NC5=O)C6=O)CC4)cc3)cn([C@H]3CC[C@H](O)CC3)c2n1] |
| I-29 | 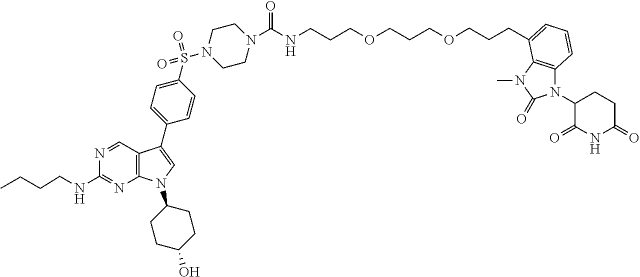 [SMILES: CCCCNc1ncc2c(-c3ccc(S(=O)(=O)N4CCN(C(=O)NCCCOCCCOCCCc5cccc6c5n(C)c(=O)n6C5CCC(=O)NC5=O)CC4)cc3)cn([C@H]3CC[C@H](O)CC3)c2n1] |
| I-30 | 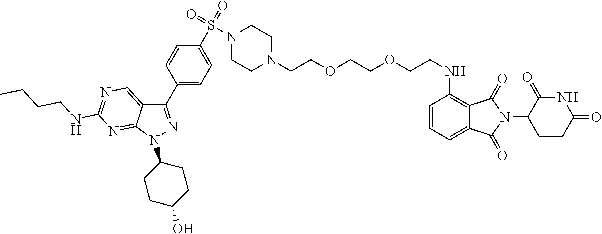 [SMILES: CCCCNc1ncc2c(-c3ccc(S(=O)(=O)N4CCN(CCOCCOCCNc5cccc6c5C(=O)N(C5CCC(=O)NC5=O)C6=O)CC4)cc3)nn([C@H]3CC[C@H](O)CC3)c2n1] |
| I-31 | 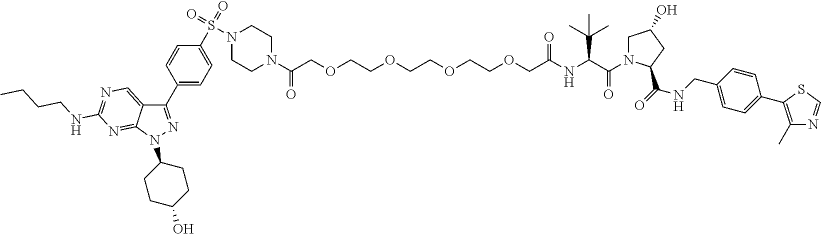 [SMILES: CCCCNc1ncc2c(-c3ccc(S(=O)(=O)N4CCN(C(=O)COCCOCCOCCOCC(=O)N[C@H](C(=O)N5C[C@H](O)C[C@H]5C(=O)NCc5ccc(-c6scnc6C)cc5)C(C)(C)C)CC4)cc3)nn([C@H]3CC[C@H](O)CC3)c2n1] |
| I-32 | 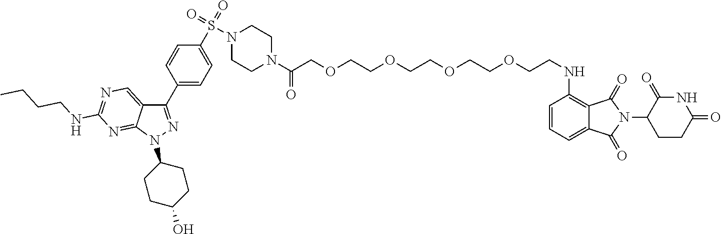 [SMILES: CCCCNc1ncc2c(-c3ccc(S(=O)(=O)N4CCN(C(=O)COCCOCCOCCOCCNc5cccc6c5C(=O)N(C5CCC(=O)NC5=O)C6=O)CC4)cc3)nn([C@H]3CC[C@H](O)CC3)c2n1] |
| I-33 | 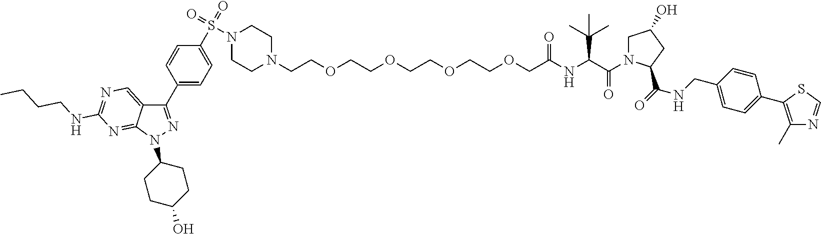 [SMILES: CCCCNc1ncc2c(-c3ccc(S(=O)(=O)N4CCN(CCOCCOCCOCCOCC(=O)N[C@H](C(=O)N5C[C@H](O)C[C@H]5C(=O)NCc5ccc(-c6scnc6C)cc5)C(C)(C)C)CC4)cc3)nn([C@H]3CC[C@H](O)CC3)c2n1] |
| I-34 | 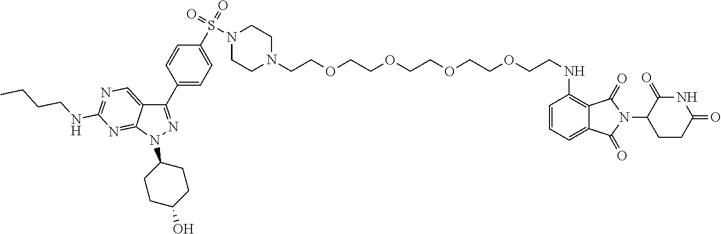 [SMILES: CCCCNc1ncc2c(-c3ccc(S(=O)(=O)N4CCN(CCOCCOCCOCCOCCNc5cccc6c5C(=O)N(C5CCC(=O)NC5=O)C6=O)CC4)cc3)nn([C@H]3CC[C@H](O)CC3)c2n1] |
| I-35 | 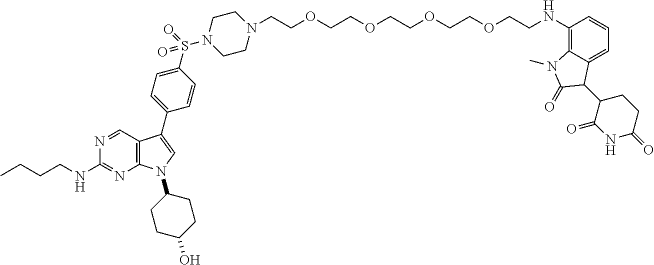 |
| I-36 | 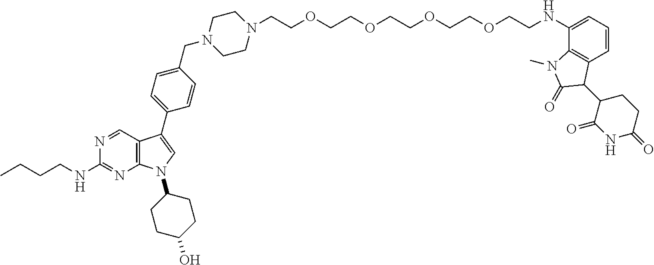 |
| I-37 | 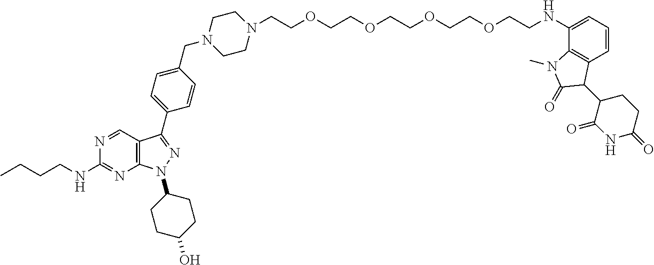 |
| I-38 | 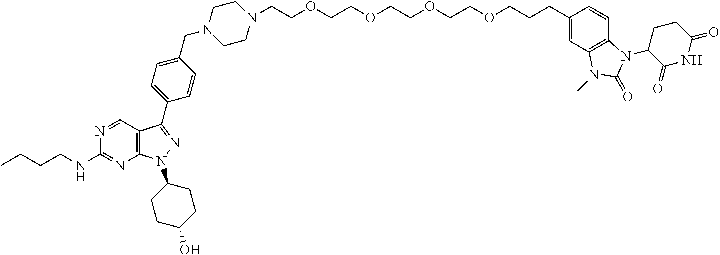 [SMILES: CCCCNc1ncc2c(-c3ccc(CN4CCN(CCOCCOCCOCCOCCCc5ccc6c(c5)n(C)c(=O)n6C5CCC(=O)NC5=O)CC4)cc3)nn([C@H]3CC[C@H](O)CC3)c2n1] |
| I-39 | 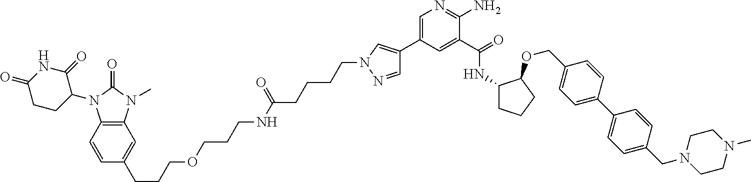 [SMILES: CN1CCN(Cc2ccc(-c3ccc(CO[C@H]4CCC[C@@H]4NC(=O)c4cc(-c5cnn(CCCCC(=O)NCCCOCCCc6ccc7c(c6)n(C)c(=O)n7C6CCC(=O)NC6=O)c5)cnc4N)cc3)cc2)CC1] |
| I-40 | 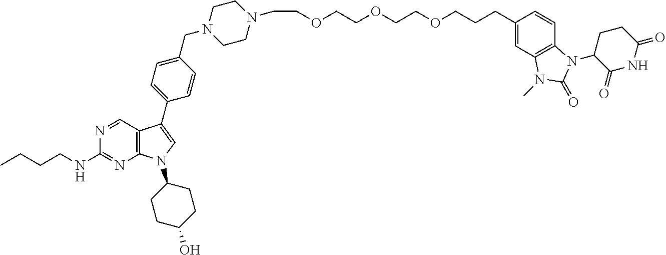 [SMILES: CCCCNc1ncc2c(-c3ccc(CN4CCN(CCOCCOCCOCCCc5ccc6c(c5)n(C)c(=O)n6C5CCC(=O)NC5=O)CC4)cc3)cn([C@H]3CC[C@H](O)CC3)c2n1] |
| I-41 | 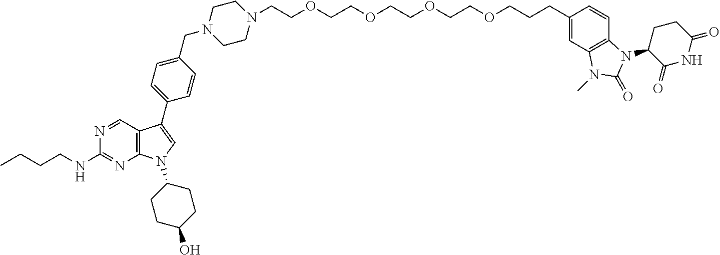 [SMILES: CCCCNc1ncc2c(-c3ccc(CN4CCN(CCOCCOCCOCCOCCCc5ccc6c(c5)n(C)c(=O)n6[C@H]5CCC(=O)NC5=O)CC4)cc3)cn([C@H]3CC[C@H](O)CC3)c2n1] |
| I-42 | 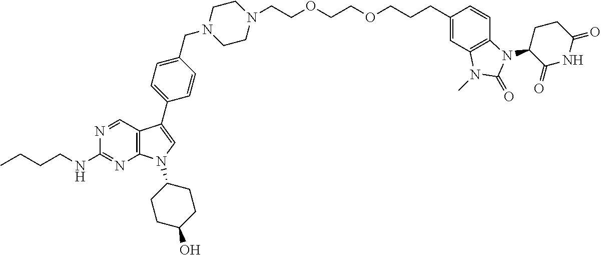 [SMILES: CCCCNc1ncc2c(-c3ccc(CN4CCN(CCOCCOCCCc5ccc6c(c5)n(C)c(=O)n6[C@H]5CCC(=O)NC5=O)CC4)cc3)cn([C@H]3CC[C@H](O)CC3)c2n1] |
| I-43 | 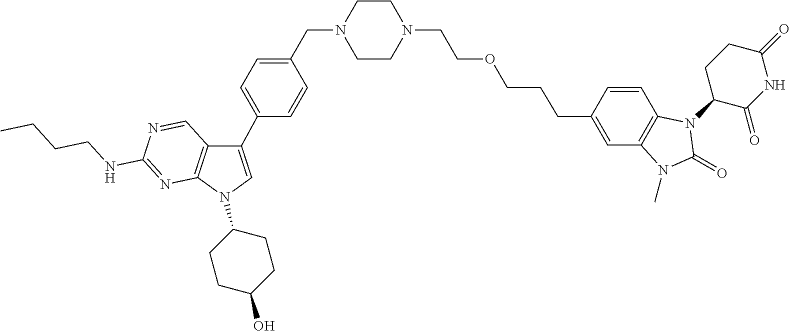 [SMILES: CCCCNc1ncc2c(-c3ccc(CN4CCN(CCOCCCc5ccc6c(c5)n(C)c(=O)n6[C@H]5CCC(=O)NC5=O)CC4)cc3)cn([C@H]3CC[C@H](O)CC3)c2n1] |
| I-44 | 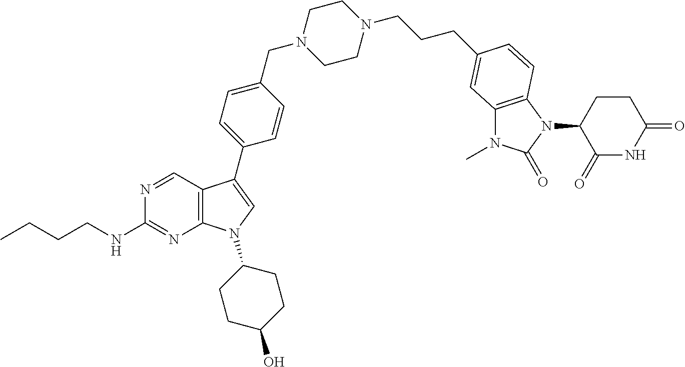 [SMILES: CCCCNc1ncc2c(-c3ccc(CN4CCN(CCCc5ccc6c(c5)n(C)c(=O)n6[C@H]5CCC(=O)NC5=O)CC4)cc3)cn([C@H]3CC[C@H](O)CC3)c2n1] |
| I-45 | 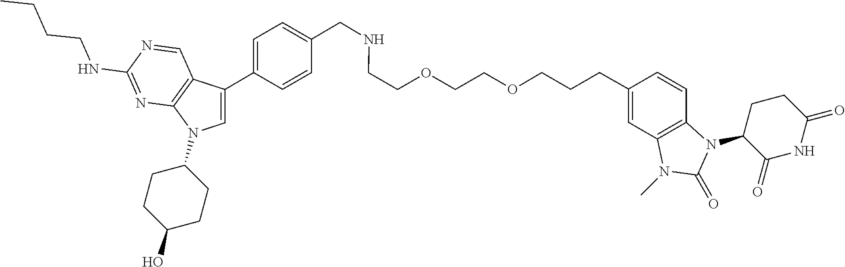 [SMILES: CCCCNc1ncc2c(-c3ccc(CNCCOCCOCCCc4ccc5c(c4)n(C)c(=O)n5[C@H]4CCC(=O)NC4=O)cc3)cn([C@H]3CC[C@H](O)CC3)c2n1] |
| I-46 | 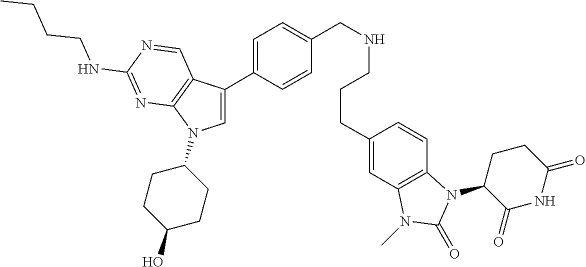 [SMILES: CCCCNc1ncc2c(-c3ccc(CNCCCc4ccc5c(c4)n(C)c(=O)n5[C@H]4CCC(=O)NC4=O)cc3)cn([C@H]3CC[C@H](O)CC3)c2n1] |
| I-47 | 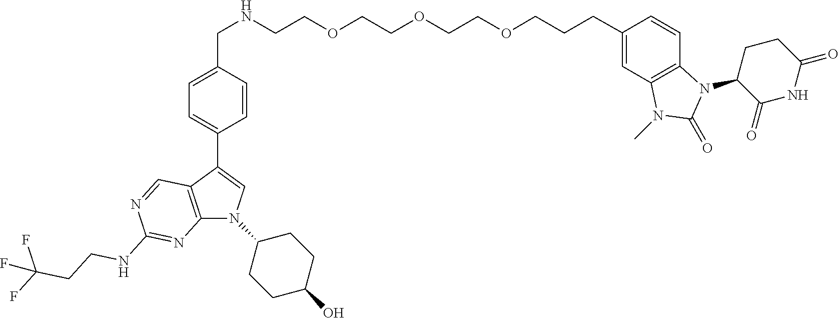 [SMILES: Cn1c(=O)n([C@H]2CCC(=O)NC2=O)c2ccc(CCCOCCOCCOCCNCc3ccc(-c4cn([C@H]5CC[C@H](O)CC5)c5nc(NCCC(F)(F)F)ncc45)cc3)cc21] |
| I-48 | 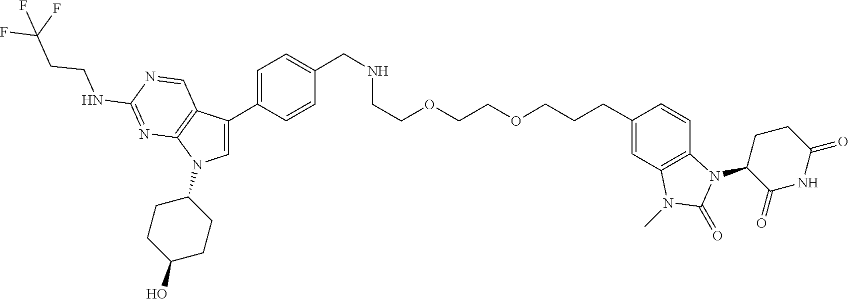 [SMILES: Cn1c(=O)n([C@H]2CCC(=O)NC2=O)c2ccc(CCCOCCOCCNCc3ccc(-c4cn([C@H]5CC[C@H](O)CC5)c5nc(NCCC(F)(F)F)ncc45)cc3)cc21] |
| I-49 | 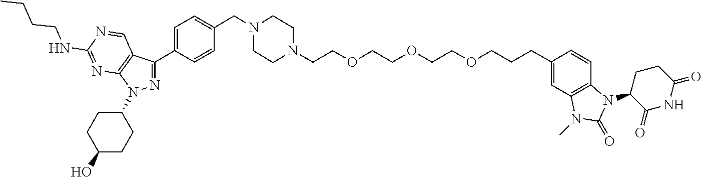 [SMILES: CCCCNc1ncc2c(-c3ccc(CN4CCN(CCOCCOCCOCCCc5ccc6c(c5)n(C)c(=O)n6[C@H]5CCC(=O)NC5=O)CC4)cc3)nn([C@H]3CC[C@H](O)CC3)c2n1] |
| I-50 | 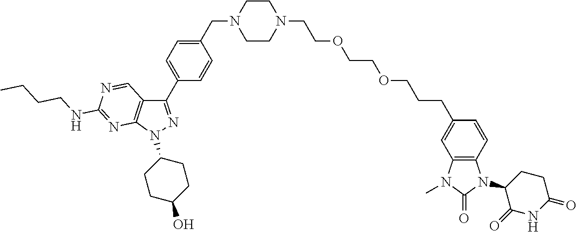 [SMILES: CCCCNc1ncc2c(-c3ccc(CN4CCN(CCOCCOCCCc5ccc6c(c5)n(C)c(=O)n6[C@H]5CCC(=O)NC5=O)CC4)cc3)nn([C@H]3CC[C@H](O)CC3)c2n1] |
| I-51 | 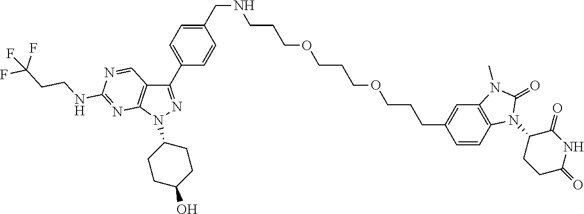 [SMILES: Cn1c(=O)n([C@H]2CCC(=O)NC2=O)c2ccc(CCCOCCCOCCCNCc3ccc(-c4nn([C@H]5CC[C@H](O)CC5)c5nc(NCCC(F)(F)F)ncc45)cc3)cc21] |
| I-52 | 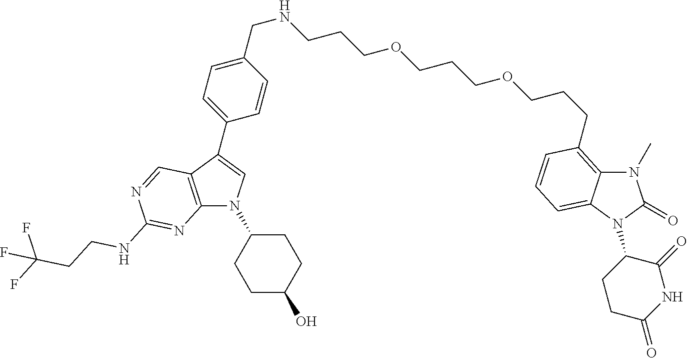 [SMILES: Cn1c(=O)n([C@H]2CCC(=O)NC2=O)c2cccc(CCCOCCCOCCCNCc3ccc(-c4cn([C@H]5CC[C@H](O)CC5)c5nc(NCCC(F)(F)F)ncc45)cc3)c21] |
| I-53 | 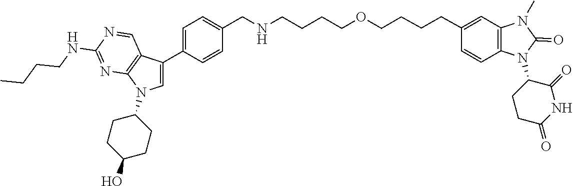 [SMILES: CCCCNc1ncc2c(-c3ccc(CNCCCCOCCCCc4ccc5c(c4)n(C)c(=O)n5[C@H]4CCC(=O)NC4=O)cc3)cn([C@H]3CC[C@H](O)CC3)c2n1] |
| I-54 | 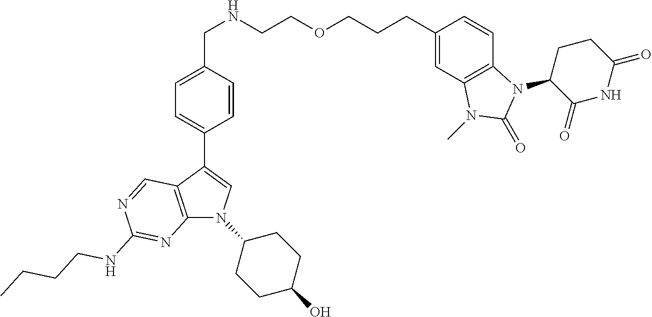 [SMILES: CCCCNc1ncc2c(-c3ccc(CNCCOCCCc4ccc5c(c4)n(C)c(=O)n5[C@H]4CCC(=O)NC4=O)cc3)cn([C@H]3CC[C@H](O)CC3)c2n1] |
| I-55 | 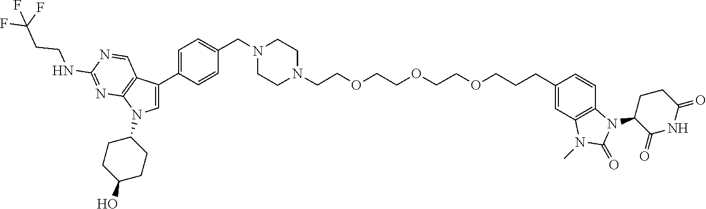 [SMILES: Cn1c(=O)n([C@H]2CCC(=O)NC2=O)c2ccc(CCCOCCOCCOCCN3CCN(Cc4ccc(-c5cn([C@H]6CC[C@H](O)CC6)c6nc(NCCC(F)(F)F)ncc56)cc4)CC3)cc21] |
| I-56 | 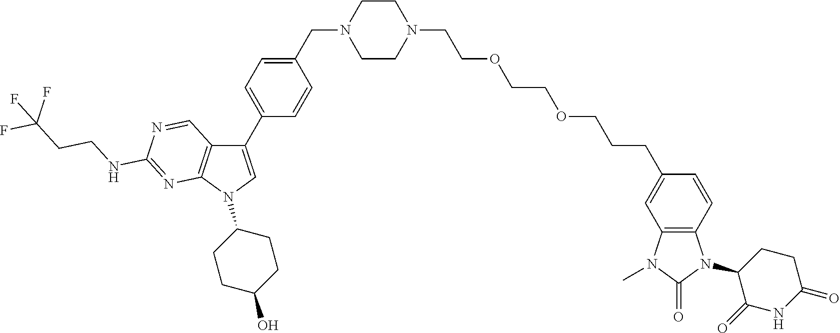 [SMILES: Cn1c(=O)n([C@H]2CCC(=O)NC2=O)c2ccc(CCCOCCOCCN3CCN(Cc4ccc(-c5cn([C@H]6CC[C@H](O)CC6)c6nc(NCCC(F)(F)F)ncc56)cc4)CC3)cc21] |
| I-57 | 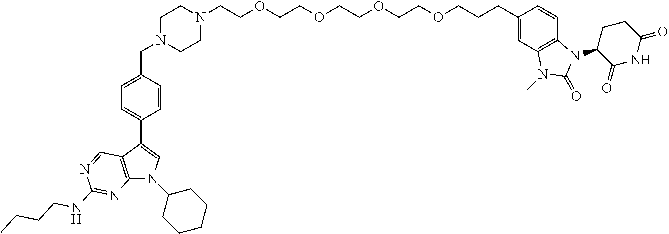 [SMILES: CCCCNc1ncc2c(-c3ccc(CN4CCN(CCOCCOCCOCCOCCCc5ccc6c(c5)n(C)c(=O)n6[C@H]5CCC(=O)NC5=O)CC4)cc3)cn(C3CCCCC3)c2n1] |
| I-58 | 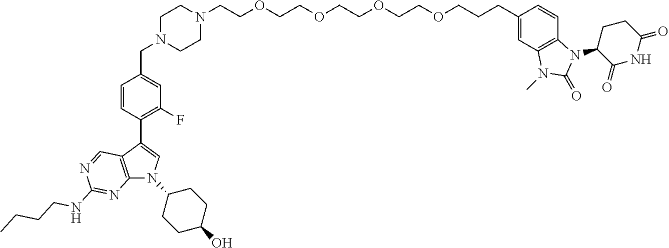 [SMILES: CCCCNc1ncc2c(-c3ccc(CN4CCN(CCOCCOCCOCCOCCCc5ccc6c(c5)n(C)c(=O)n6[C@H]5CCC(=O)NC5=O)CC4)cc3F)cn([C@H]3CC[C@H](O)CC3)c2n1] |
| I-59 | 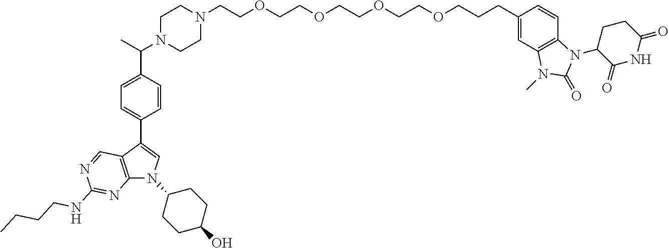 [SMILES: CCCCNc1ncc2c(-c3ccc(C(C)N4CCN(CCOCCOCCOCCOCCCc5ccc6c(c5)n(C)c(=O)n6C5CCC(=O)NC5=O)CC4)cc3)cn([C@H]3CC[C@H](O)CC3)c2n1] |
| I-60 | 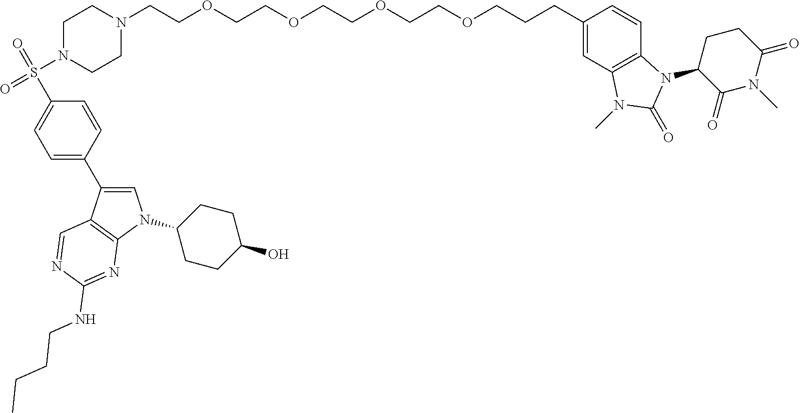 [SMILES: CCCCNc1ncc2c(-c3ccc(S(=O)(=O)N4CCN(CCOCCOCCOCCOCCCc5ccc6c(c5)n(C)c(=O)n6[C@H]5CCC(=O)N(C)C5=O)CC4)cc3)cn([C@H]3CC[C@H](O)CC3)c2n1] |
| I-61 | 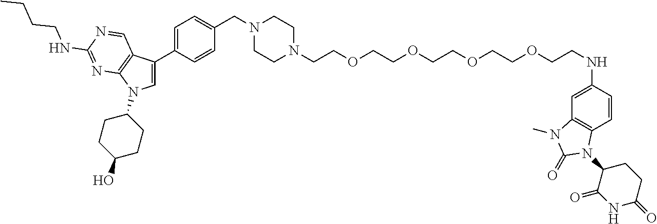 [SMILES: CCCCNc1ncc2c(-c3ccc(CN4CCN(CCOCCOCCOCCOCCNc5ccc6c(c5)n(C)c(=O)n6[C@H]5CCC(=O)NC5=O)CC4)cc3)cn([C@H]3CC[C@H](O)CC3)c2n1] |
| I-62 | 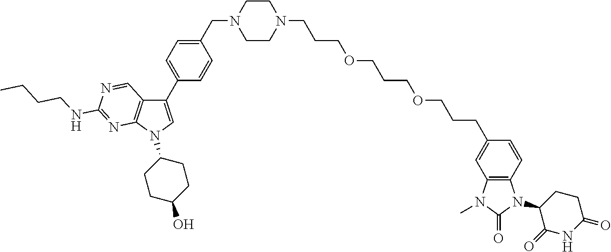 [SMILES: CCCCNc1ncc2c(-c3ccc(CN4CCN(CCCOCCCOCCCc5ccc6c(c5)n(C)c(=O)n6[C@H]5CCC(=O)NC5=O)CC4)cc3)cn([C@H]3CC[C@H](O)CC3)c2n1] |
| I-63 | 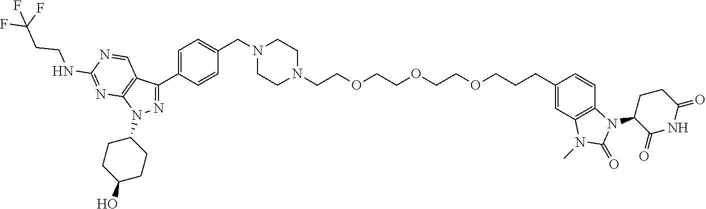 [SMILES: Cn1c(=O)n([C@H]2CCC(=O)NC2=O)c2ccc(CCCOCCOCCOCCN3CCN(Cc4ccc(-c5nn([C@H]6CC[C@H](O)CC6)c6nc(NCCC(F)(F)F)ncc56)cc4)CC3)cc21] |
| I-64 | 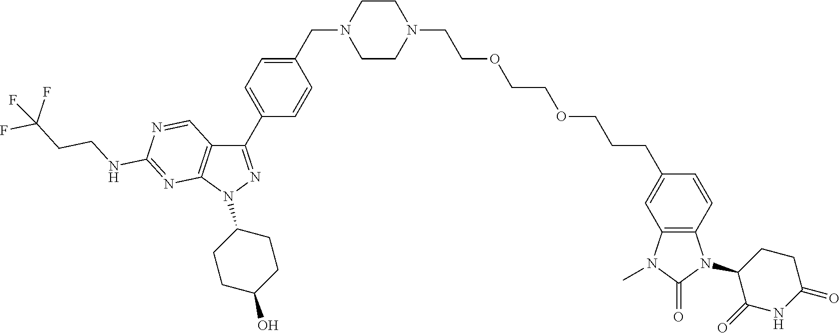 [SMILES: Cn1c(=O)n([C@H]2CCC(=O)NC2=O)c2ccc(CCCOCCOCCN3CCN(Cc4ccc(-c5nn([C@H]6CC[C@H](O)CC6)c6nc(NCCC(F)(F)F)ncc56)cc4)CC3)cc21] |
| I-65 | 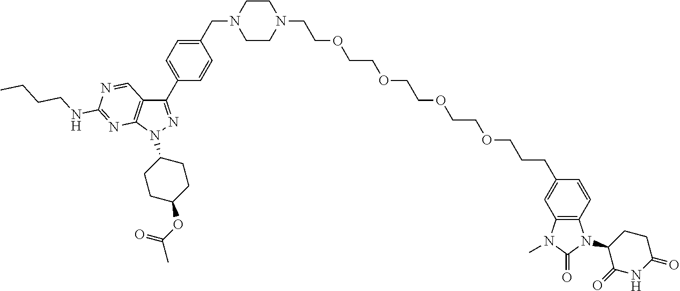 [SMILES: CCCCNc1ncc2c(-c3ccc(CN4CCN(CCOCCOCCOCCOCCCc5ccc6c(c5)n(C)c(=O)n6[C@H]5CCC(=O)NC5=O)CC4)cc3)nn([C@H]3CC[C@H](OC(C)=O)CC3)c2n1] |
| I-66 | 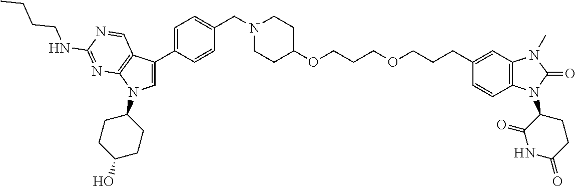 [SMILES: CCCCNc1ncc2c(-c3ccc(CN4CCC(OCCCOCCCc5ccc6c(c5)n(C)c(=O)n6[C@H]5CCC(=O)NC5=O)CC4)cc3)cn([C@H]3CC[C@H](O)CC3)c2n1] |
| I-67 | 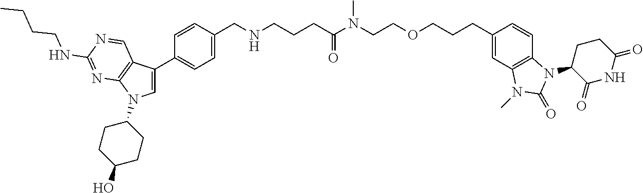 [SMILES: CCCCNc1ncc2c(-c3ccc(CNCCCC(=O)N(C)CCOCCCc4ccc5c(c4)n(C)c(=O)n5[C@H]4CCC(=O)NC4=O)cc3)cn([C@H]3CC[C@H](O)CC3)c2n1] |
| I-68 | 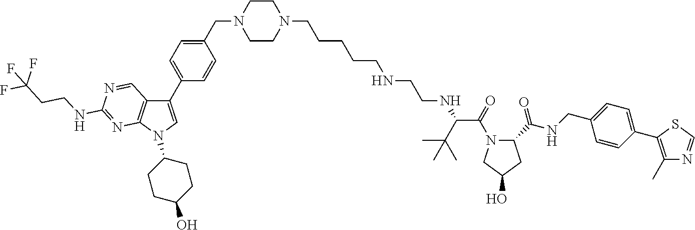 [SMILES: Cc1ncsc1-c1ccc(CNC(=O)[C@@H]2C[C@@H](O)CN2C(=O)[C@@H](NCCNCCCCCN2CCN(Cc3ccc(-c4cn([C@H]5CC[C@H](O)CC5)c5nc(NCCC(F)(F)F)ncc45)cc3)CC2)C(C)(C)C)cc1] |
| I-69 | 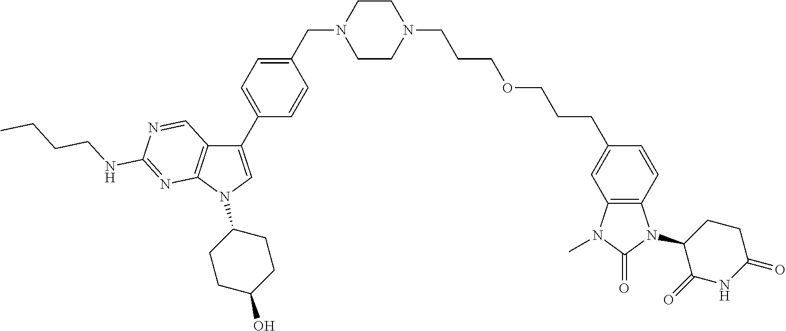 [SMILES: CCCCNc1ncc2c(-c3ccc(CN4CCN(CCCOCCCc5ccc6c(c5)n(C)c(=O)n6[C@H]5CCC(=O)NC5=O)CC4)cc3)cn([C@H]3CC[C@H](O)CC3)c2n1] |
| I-70 | 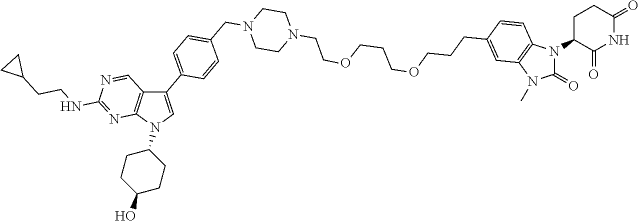 [SMILES: Cn1c(=O)n([C@H]2CCC(=O)NC2=O)c2ccc(CCCOCCCOCCN3CCN(Cc4ccc(-c5cn([C@H]6CC[C@H](O)CC6)c6nc(NCCC7CC7)ncc56)cc4)CC3)cc21] |
| I-71 | 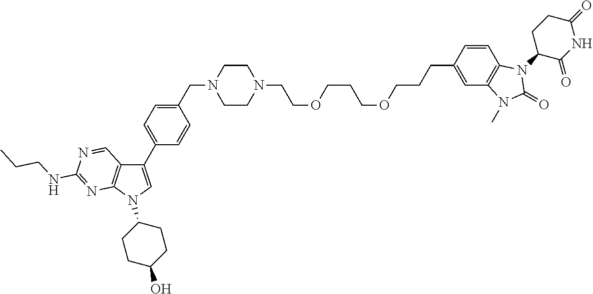 [SMILES: CCCNc1ncc2c(-c3ccc(CN4CCN(CCOCCCOCCCc5ccc6c(c5)n(C)c(=O)n6[C@H]5CCC(=O)NC5=O)CC4)cc3)cn([C@H]3CC[C@H](O)CC3)c2n1] |
| I-72 | 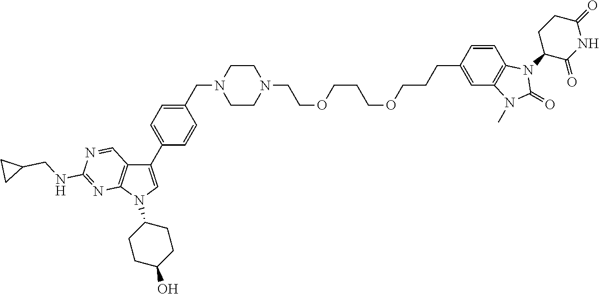 [SMILES: Cn1c(=O)n([C@H]2CCC(=O)NC2=O)c2ccc(CCCOCCCOCCN3CCN(Cc4ccc(-c5cn([C@H]6CC[C@H](O)CC6)c6nc(NCC7CC7)ncc56)cc4)CC3)cc21] |
| I-73 | 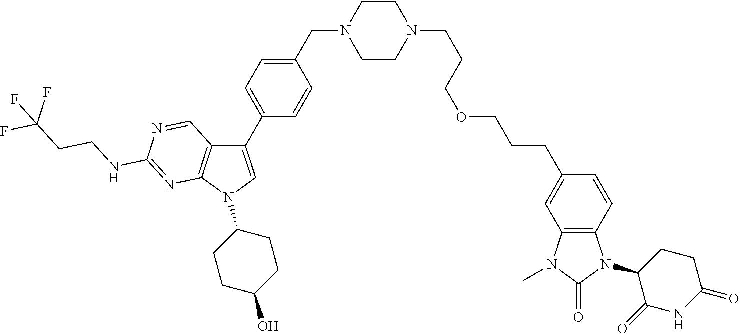 [SMILES: Cn1c(=O)n([C@H]2CCC(=O)NC2=O)c2ccc(CCCOCCCN3CCN(Cc4ccc(-c5cn([C@H]6CC[C@H](O)CC6)c6nc(NCCC(F)(F)F)ncc56)cc4)CC3)cc21] |
| I-74 | 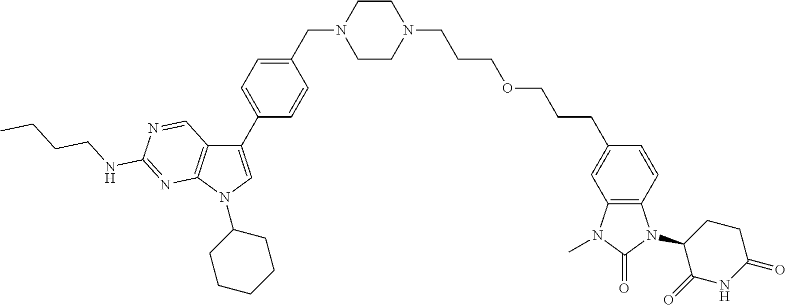 [SMILES: CCCCNc1ncc2c(-c3ccc(CN4CCN(CCCOCCCc5ccc6c(c5)n(C)c(=O)n6[C@H]5CCC(=O)NC5=O)CC4)cc3)cn(C3CCCCC3)c2n1] |
| I-75 | 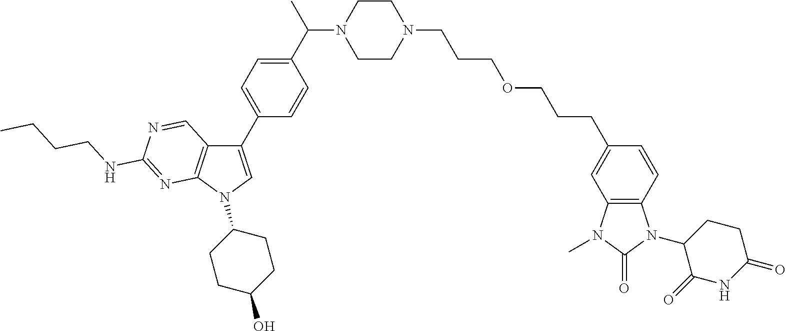 [SMILES: CCCCNc1ncc2c(-c3ccc(C(C)N4CCN(CCCOCCCc5ccc6c(c5)n(C)c(=O)n6C5CCC(=O)NC5=O)CC4)cc3)cn([C@H]3CC[C@H](O)CC3)c2n1] |
| I-76 | 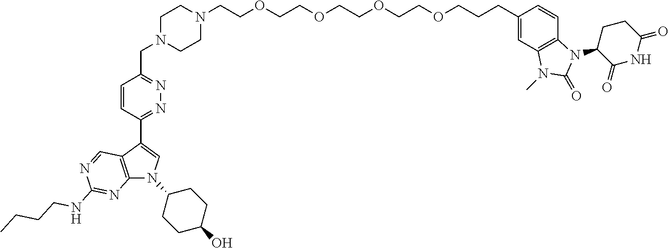 [SMILES: CCCCNc1ncc2c(-c3ccc(CN4CCN(CCOCCOCCOCCOCCCc5ccc6c(c5)n(C)c(=O)n6[C@H]5CCC(=O)NC5=O)CC4)nn3)cn([C@H]3CC[C@H](O)CC3)c2n1] |
| I-77 | 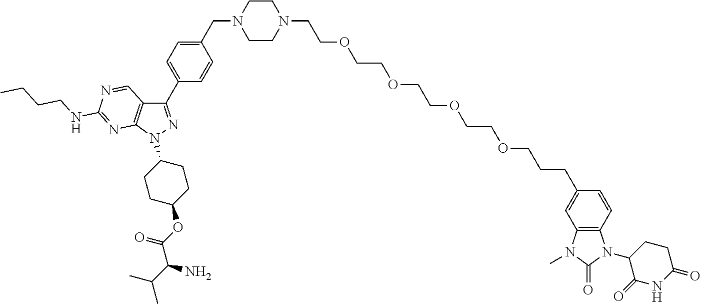 [SMILES: CCCCNc1ncc2c(-c3ccc(CN4CCN(CCOCCOCCOCCOCCCc5ccc6c(c5)n(C)c(=O)n6C5CCC(=O)NC5=O)CC4)cc3)nn([C@H]3CC[C@H](OC(=O)[C@@H](N)C(C)C)CC3)c2n1] |
| I-78 | 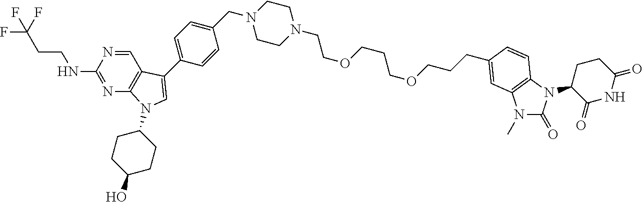 [SMILES: Cn1c(=O)n([C@H]2CCC(=O)NC2=O)c2ccc(CCCOCCCOCCN3CCN(Cc4ccc(-c5cn([C@H]6CC[C@H](O)CC6)c6nc(NCCC(F)(F)F)ncc56)cc4)CC3)cc21] |
| I-79 | 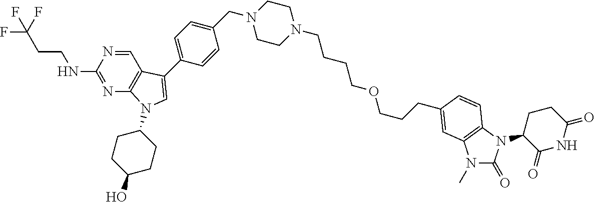 [SMILES: Cn1c(=O)n([C@H]2CCC(=O)NC2=O)c2ccc(CCCOCCCCN3CCN(Cc4ccc(-c5cn([C@H]6CC[C@H](O)CC6)c6nc(NCCC(F)(F)F)ncc56)cc4)CC3)cc21] |
| I-80 | 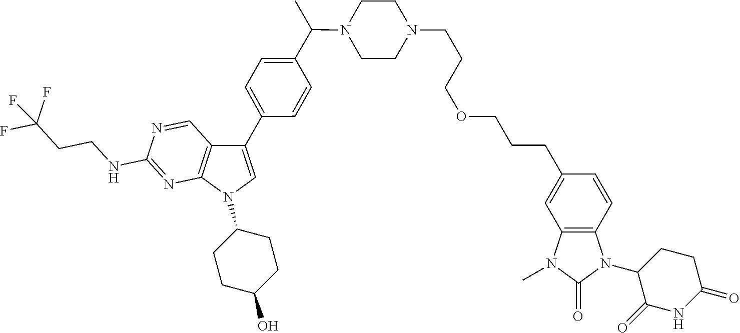 [SMILES: CC(c1ccc(-c2cn([C@H]3CC[C@H](O)CC3)c3nc(NCCC(F)(F)F)ncc23)cc1)N1CCN(CCCOCCCc2ccc3c(c2)n(C)c(=O)n3C2CCC(=O)NC2=O)CC1] |
| I-81 | 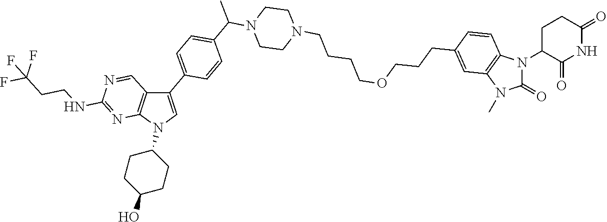 [SMILES: CC(c1ccc(-c2cn([C@H]3CC[C@H](O)CC3)c3nc(NCCC(F)(F)F)ncc23)cc1)N1CCN(CCCCOCCCc2ccc3c(c2)n(C)c(=O)n3C2CCC(=O)NC2=O)CC1] |
| I-82 | 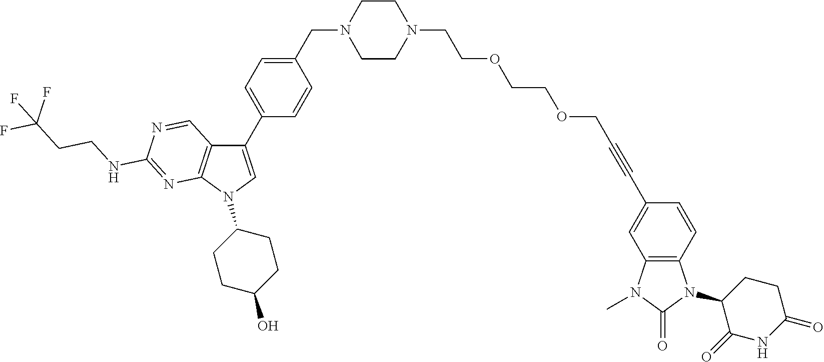 [SMILES: Cn1c(=O)n([C@H]2CCC(=O)NC2=O)c2ccc(C#CCOCCOCCN3CCN(Cc4ccc(-c5cn([C@H]6CC[C@H](O)CC6)c6nc(NCCC(F)(F)F)ncc56)cc4)CC3)cc21] |
| I-83 | 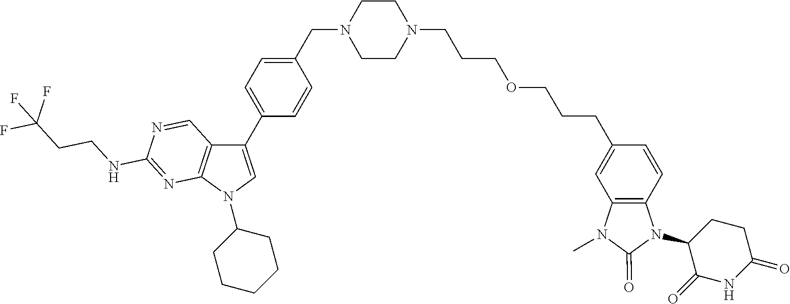 [SMILES: Cn1c(=O)n([C@H]2CCC(=O)NC2=O)c2ccc(CCCOCCCN3CCN(Cc4ccc(-c5cn(C6CCCCC6)c6nc(NCCC(F)(F)F)ncc56)cc4)CC3)cc21] |
| I-84 | 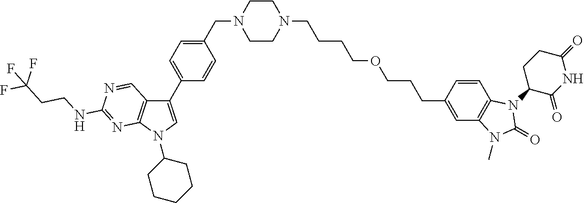 [SMILES: Cn1c(=O)n([C@H]2CCC(=O)NC2=O)c2ccc(CCCOCCCCN3CCN(Cc4ccc(-c5cn(C6CCCCC6)c6nc(NCCC(F)(F)F)ncc56)cc4)CC3)cc21] |
| I-85 | 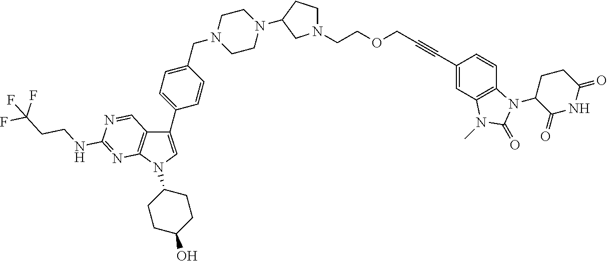 [SMILES: Cn1c(=O)n(C2CCC(=O)NC2=O)c2ccc(C#CCOCCN3CCC(N4CCN(Cc5ccc(-c6cn([C@H]7CC[C@H](O)CC7)c7nc(NCCC(F)(F)F)ncc67)cc5)CC4)C3)cc21] |
| I-86 | 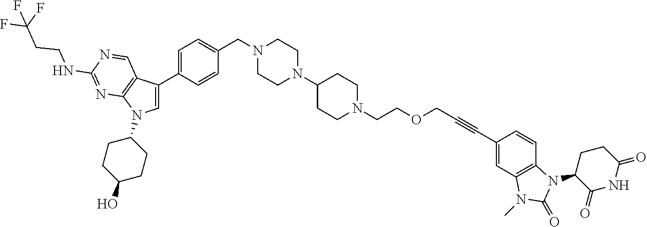 [SMILES: Cn1c(=O)n([C@H]2CCC(=O)NC2=O)c2ccc(C#CCOCCN3CCC(N4CCN(Cc5ccc(-c6cn([C@H]7CC[C@H](O)CC7)c7nc(NCCC(F)(F)F)ncc67)cc5)CC4)CC3)cc21] |
| I-87 | 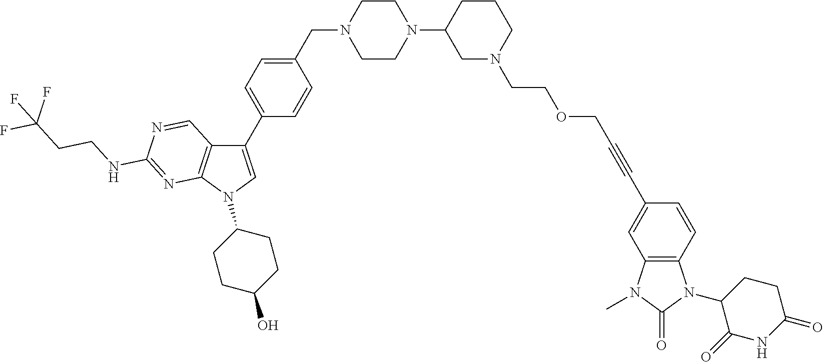 [SMILES: Cn1c(=O)n(C2CCC(=O)NC2=O)c2ccc(C#CCOCCN3CCCC(N4CCN(Cc5ccc(-c6cn([C@H]7CC[C@H](O)CC7)c7nc(NCCC(F)(F)F)ncc67)cc5)CC4)C3)cc21] |
| I-88 | 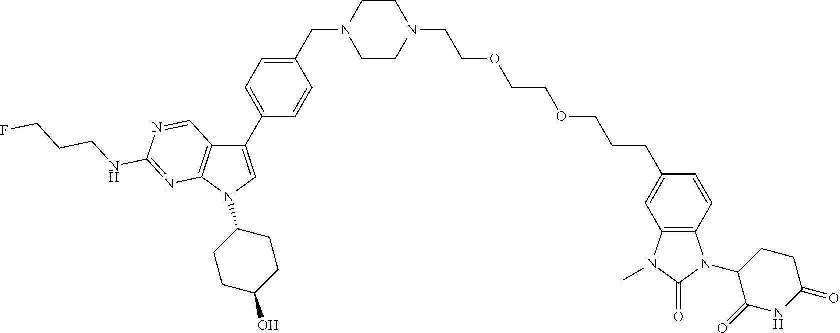 [SMILES: Cn1c(=O)n(C2CCC(=O)NC2=O)c2ccc(CCCOCCOCCN3CCN(Cc4ccc(-c5cn([C@H]6CC[C@H](O)CC6)c6nc(NCCCF)ncc56)cc4)CC3)cc21] |
| I-89 | 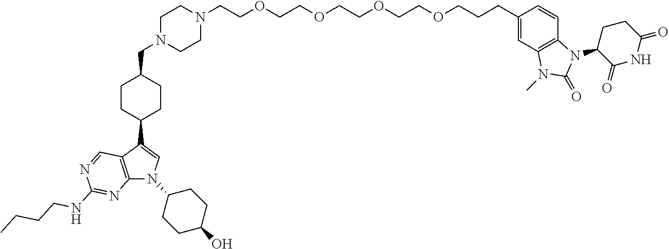 [SMILES: CCCCNc1ncc2c(n1)n([C@H]1CC[C@H](O)CC1)cc2[C@H]1CC[C@@H](CN2CCN(CCOCCOCCOCCOCCCc3ccc4c(c3)n(C)c(=O)n4[C@H]3CCC(=O)NC3=O)CC2)CC1] |
| I-90 | 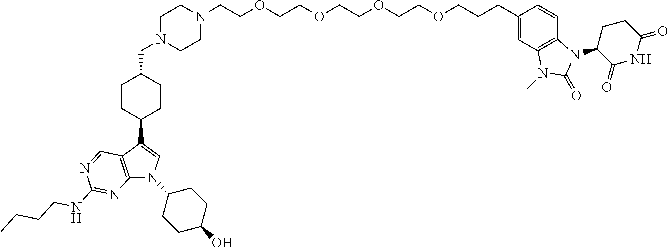 [SMILES: CCCCNc1ncc2c(n1)n([C@H]1CC[C@H](O)CC1)cc2[C@H]1CC[C@H](CN2CCN(CCOCCOCCOCCOCCCc3ccc4c(c3)n(C)c(=O)n4[C@H]3CCC(=O)NC3=O)CC2)CC1] |
| I-91 | 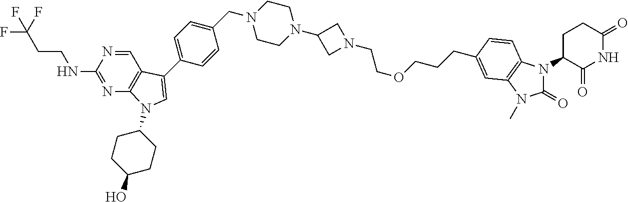 [SMILES: Cn1c(=O)n([C@H]2CCC(=O)NC2=O)c2ccc(CCCOCCN3CC(N4CCN(Cc5ccc(-c6cn([C@H]7CC[C@H](O)CC7)c7nc(NCCC(F)(F)F)ncc67)cc5)CC4)C3)cc21] |
| I-92 | 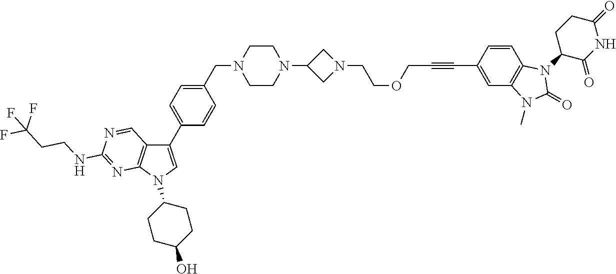 [SMILES: Cn1c(=O)n([C@H]2CCC(=O)NC2=O)c2ccc(C#CCOCCN3CC(N4CCN(Cc5ccc(-c6cn([C@H]7CC[C@H](O)CC7)c7nc(NCCC(F)(F)F)ncc67)cc5)CC4)C3)cc21] |
| I-93 | 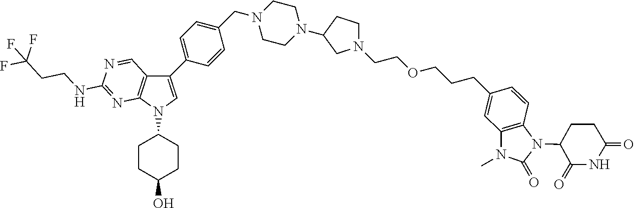 [SMILES: Cn1c(=O)n(C2CCC(=O)NC2=O)c2ccc(CCCOCCN3CCC(N4CCN(Cc5ccc(-c6cn([C@H]7CC[C@H](O)CC7)c7nc(NCCC(F)(F)F)ncc67)cc5)CC4)C3)cc21] |
| I-94 | 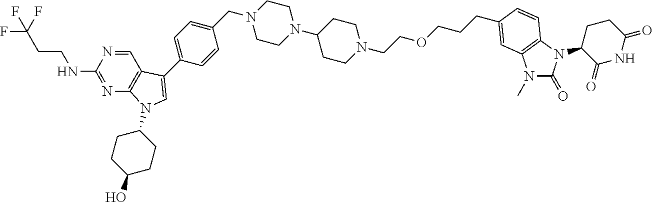 [SMILES: Cn1c(=O)n([C@H]2CCC(=O)NC2=O)c2ccc(CCCOCCN3CCC(N4CCN(Cc5ccc(-c6cn([C@H]7CC[C@H](O)CC7)c7nc(NCCC(F)(F)F)ncc67)cc5)CC4)CC3)cc21] |
| I-95 | 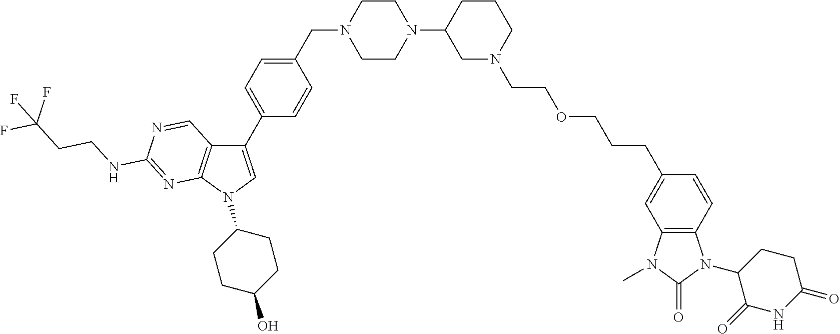 [SMILES: Cn1c(=O)n(C2CCC(=O)NC2=O)c2ccc(CCCOCCN3CCCC(N4CCN(Cc5ccc(-c6cn([C@H]7CC[C@H](O)CC7)c7nc(NCCC(F)(F)F)ncc67)cc5)CC4)C3)cc21] |
| I-96 | 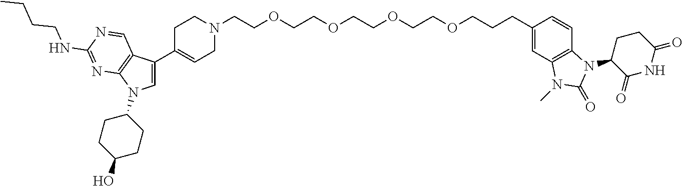 [SMILES: CCCCNc1ncc2c(C3=CCN(CCOCCOCCOCCOCCCc4ccc5c(c4)n(C)c(=O)n5[C@H]4CCC(=O)NC4=O)CC3)cn([C@H]3CC[C@H](O)CC3)c2n1] |
| I-97 | 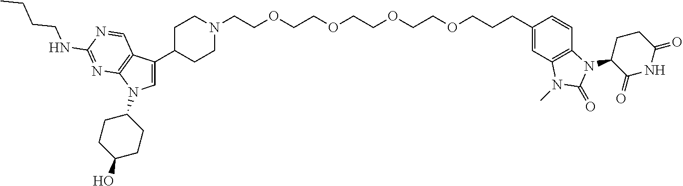 [SMILES: CCCCNc1ncc2c(C3CCN(CCOCCOCCOCCOCCCc4ccc5c(c4)n(C)c(=O)n5[C@H]4CCC(=O)NC4=O)CC3)cn([C@H]3CC[C@H](O)CC3)c2n1] |
| I-98 | 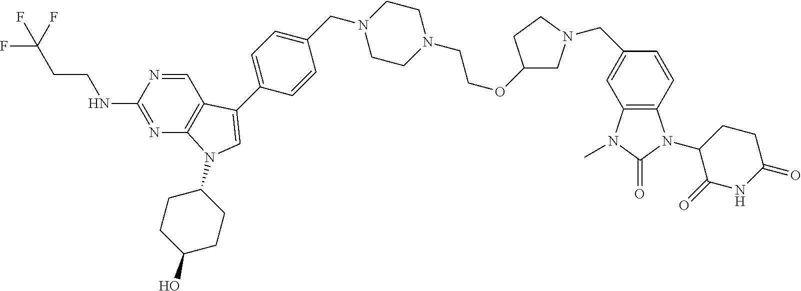 [SMILES: Cn1c(=O)n(C2CCC(=O)NC2=O)c2ccc(CN3CCC(OCCN4CCN(Cc5ccc(-c6cn([C@H]7CC[C@H](O)CC7)c7nc(NCCC(F)(F)F)ncc67)cc5)CC4)C3)cc21] |
| I-99 | 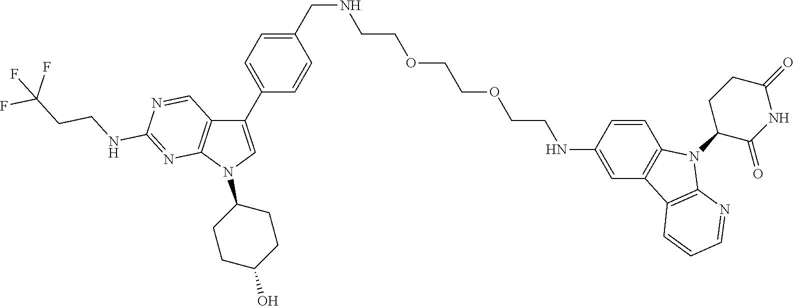 [SMILES: O=C1CC[C@H](n2c3ccc(NCCOCCOCCNCc4ccc(-c5cn([C@H]6CC[C@H](O)CC6)c6nc(NCCC(F)(F)F)ncc56)cc4)cc3c3cccnc32)C(=O)N1] |
| I-100 | 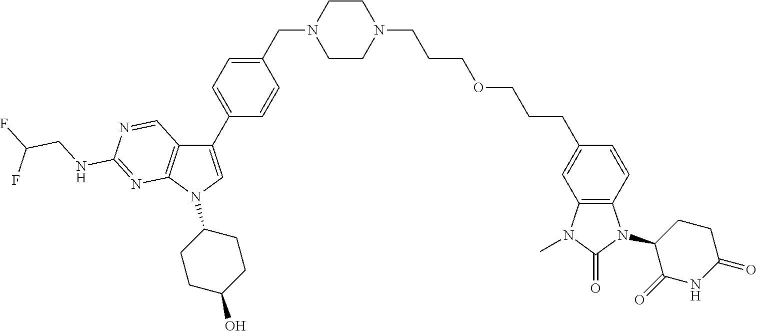 [SMILES: Cn1c(=O)n([C@H]2CCC(=O)NC2=O)c2ccc(CCCOCCCN3CCN(Cc4ccc(-c5cn([C@H]6CC[C@H](O)CC6)c6nc(NCC(F)F)ncc56)cc4)CC3)cc21] |
| I-101 | 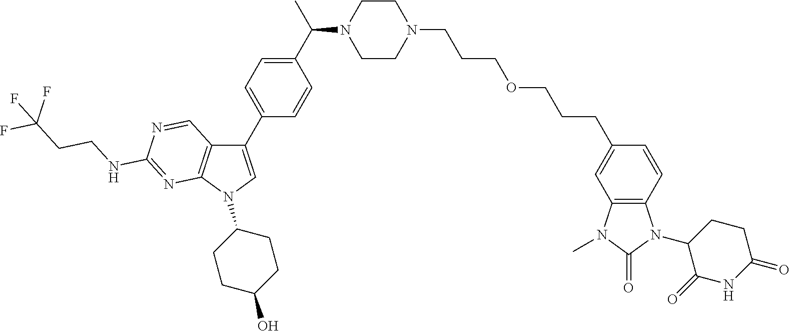 [SMILES: C[C@H](c1ccc(-c2cn([C@H]3CC[C@H](O)CC3)c3nc(NCCC(F)(F)F)ncc23)cc1)N1CCN(CCCOCCCc2ccc3c(c2)n(C)c(=O)n3C2CCC(=O)NC2=O)CC1] |
| I-102 | 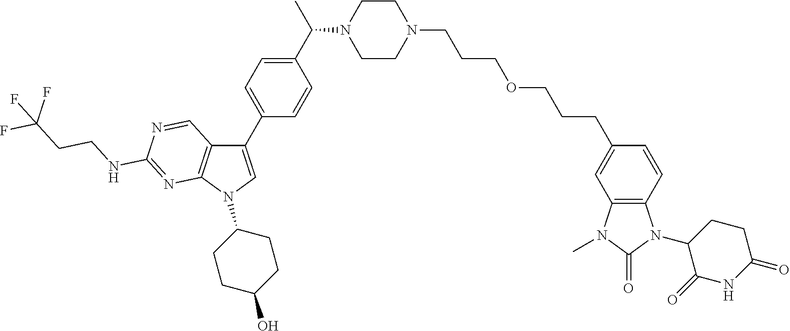 [SMILES: C[C@@H](c1ccc(-c2cn([C@H]3CC[C@H](O)CC3)c3nc(NCCC(F)(F)F)ncc23)cc1)N1CCN(CCCOCCCc2ccc3c(c2)n(C)c(=O)n3C2CCC(=O)NC2=O)CC1] |
| I-103 | 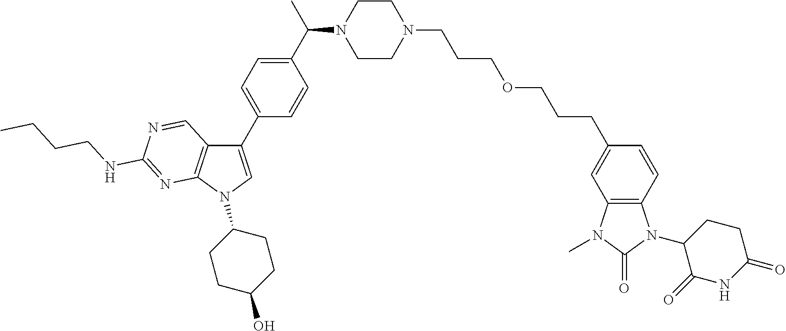 [SMILES: CCCCNc1ncc2c(-c3ccc([C@@H](C)N4CCN(CCCOCCCc5ccc6c(c5)n(C)c(=O)n6C5CCC(=O)NC5=O)CC4)cc3)cn([C@H]3CC[C@H](O)CC3)c2n1] |
| I-104 | 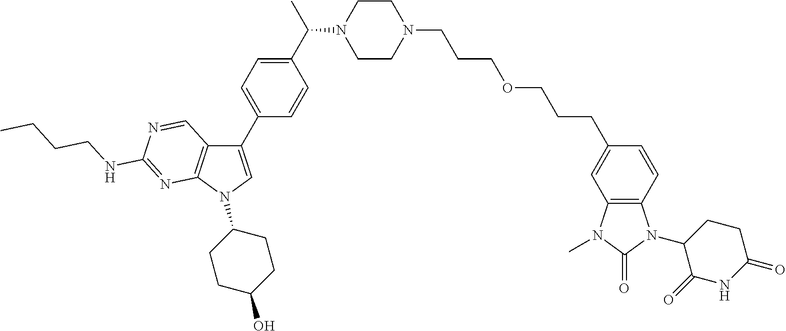 [SMILES: CCCCNc1ncc2c(-c3ccc([C@H](C)N4CCN(CCCOCCCc5ccc6c(c5)n(C)c(=O)n6C5CCC(=O)NC5=O)CC4)cc3)cn([C@H]3CC[C@H](O)CC3)c2n1] |
| I-105 | 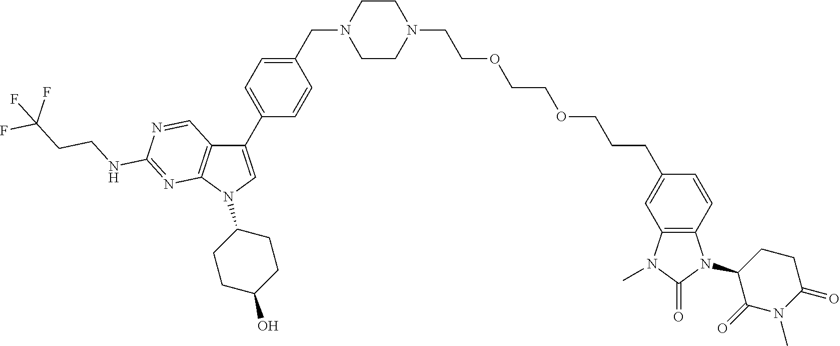 [SMILES: CN1C(=O)CC[C@H](n2c(=O)n(C)c3cc(CCCOCCOCCN4CCN(Cc5ccc(-c6cn([C@H]7CC[C@H](O)CC7)c7nc(NCCC(F)(F)F)ncc67)cc5)CC4)ccc32)C1=O] |
| I-106 | 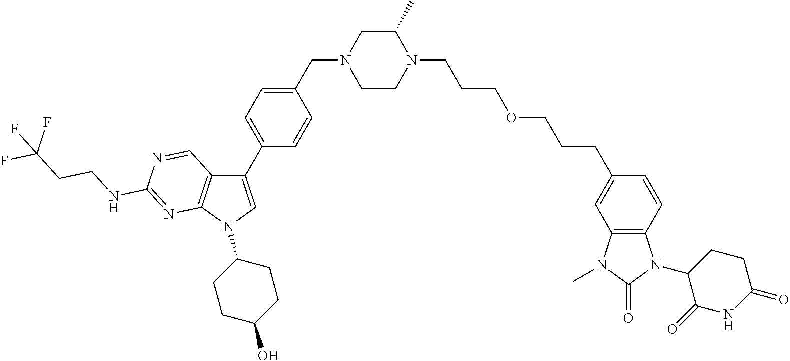 [SMILES: C[C@H]1CN(Cc2ccc(-c3cn([C@H]4CC[C@H](O)CC4)c4nc(NCCC(F)(F)F)ncc34)cc2)CCN1CCCOCCCc1ccc2c(c1)n(C)c(=O)n2C1CCC(=O)NC1=O] |
| I-107 | 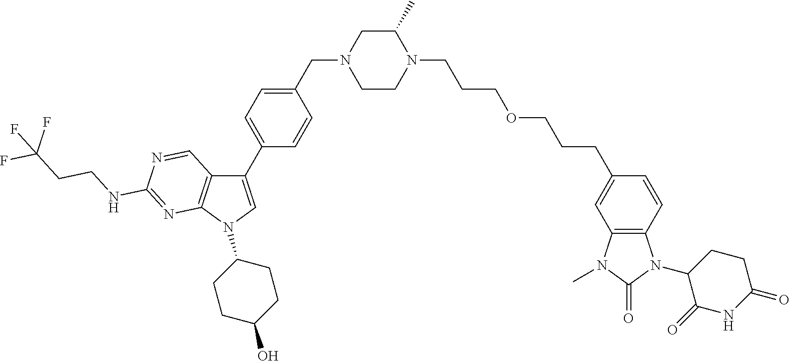 [SMILES: C[C@H]1CN(Cc2ccc(-c3cn([C@H]4CC[C@H](O)CC4)c4nc(NCCC(F)(F)F)ncc34)cc2)CCN1CCCOCCCc1ccc2c(c1)n(C)c(=O)n2C1CCC(=O)NC1=O] |
| I-108 | 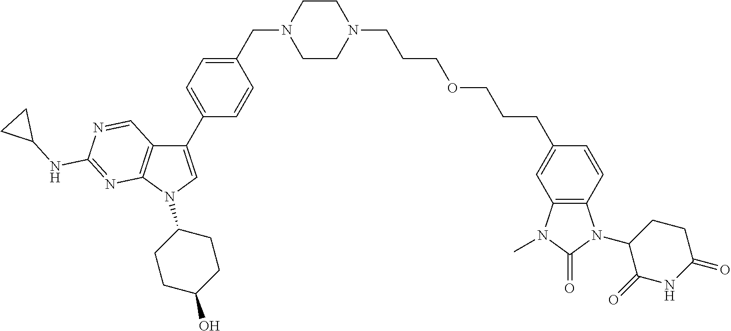 [SMILES: Cn1c(=O)n(C2CCC(=O)NC2=O)c2ccc(CCCOCCCN3CCN(Cc4ccc(-c5cn([C@H]6CC[C@H](O)CC6)c6nc(NC7CC7)ncc56)cc4)CC3)cc21] |
| I-109 | 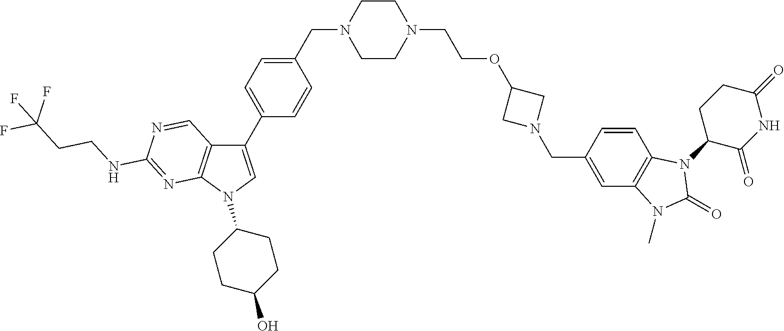 [SMILES: Cn1c(=O)n([C@H]2CCC(=O)NC2=O)c2ccc(CN3CC(OCCN4CCN(Cc5ccc(-c6cn([C@H]7CC[C@H](O)CC7)c7nc(NCCC(F)(F)F)ncc67)cc5)CC4)C3)cc21] |
| I-110 | 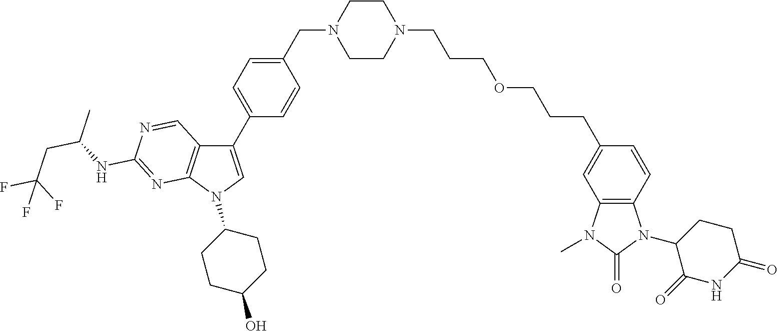 [SMILES: C[C@@H](CC(F)(F)F)Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1] |
| I-111 | 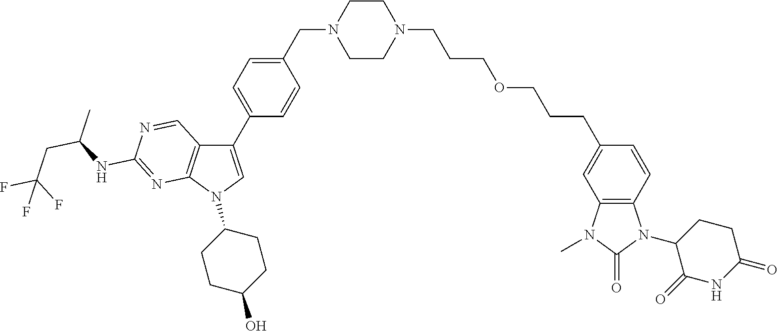 [SMILES: C[C@H](CC(F)(F)F)Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1] |
| I-112 | 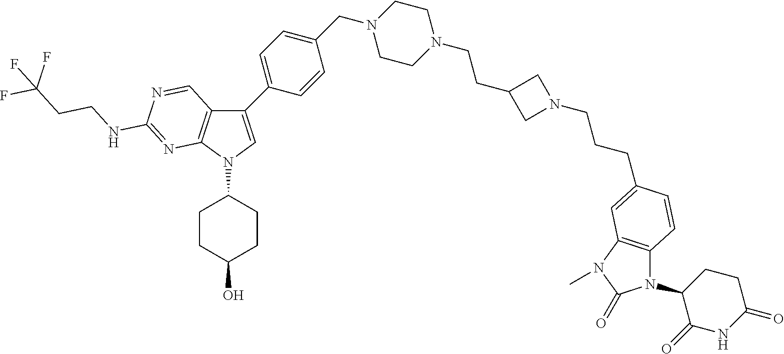 [SMILES: Cn1c(=O)n([C@H]2CCC(=O)NC2=O)c2ccc(CCCN3CC(CCN4CCN(Cc5ccc(-c6cn([C@H]7CC[C@H](O)CC7)c7nc(NCCC(F)(F)F)ncc67)cc5)CC4)C3)cc21] |
| I-113 | 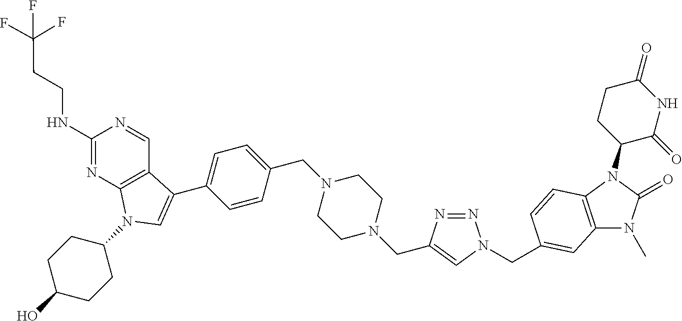 [SMILES: Cn1c(=O)n([C@H]2CCC(=O)NC2=O)c2ccc(Cn3cc(CN4CCN(Cc5ccc(-c6cn([C@H]7CC[C@H](O)CC7)c7nc(NCCC(F)(F)F)ncc67)cc5)CC4)nn3)cc21] |
| I-114 | 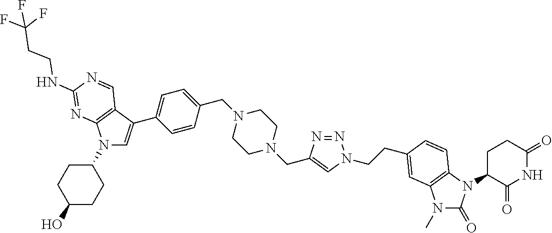 [SMILES: Cn1c(=O)n([C@H]2CCC(=O)NC2=O)c2ccc(CCn3cc(CN4CCN(Cc5ccc(-c6cn([C@H]7CC[C@H](O)CC7)c7nc(NCCC(F)(F)F)ncc67)cc5)CC4)nn3)cc21] |
| I-115 | 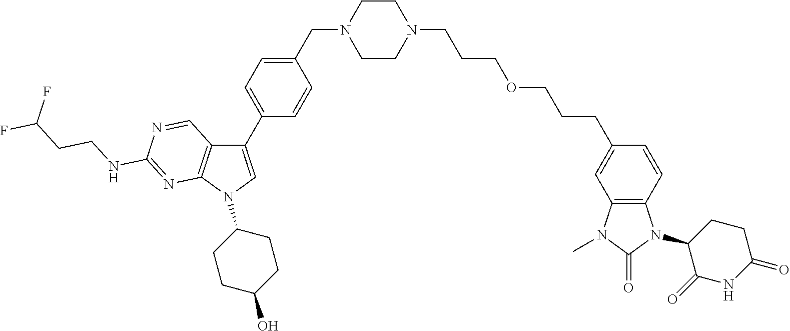 [SMILES: Cn1c(=O)n([C@H]2CCC(=O)NC2=O)c2ccc(CCCOCCCN3CCN(Cc4ccc(-c5cn([C@H]6CC[C@H](O)CC6)c6nc(NCCC(F)F)ncc56)cc4)CC3)cc21] |
| I-116 | 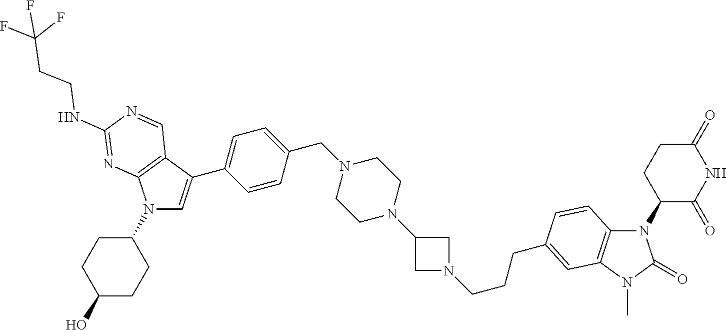 [SMILES: Cn1c(=O)n([C@H]2CCC(=O)NC2=O)c2ccc(CCCN3CC(N4CCN(Cc5ccc(-c6cn([C@H]7CC[C@H](O)CC7)c7nc(NCCC(F)(F)F)ncc67)cc5)CC4)C3)cc21] |
| I-117 | 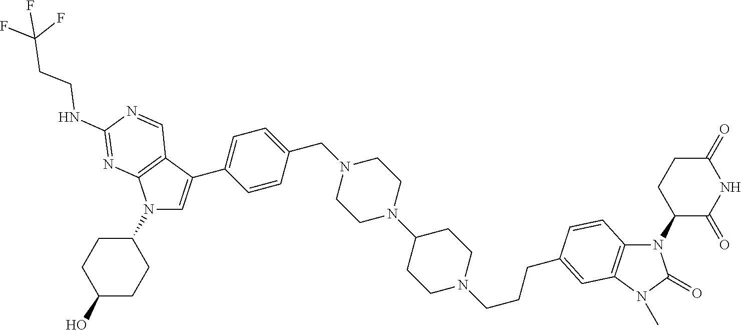 [SMILES: Cn1c(=O)n([C@H]2CCC(=O)NC2=O)c2ccc(CCCN3CCC(N4CCN(Cc5ccc(-c6cn([C@H]7CC[C@H](O)CC7)c7nc(NCCC(F)(F)F)ncc67)cc5)CC4)CC3)cc21] |
| I-118 | 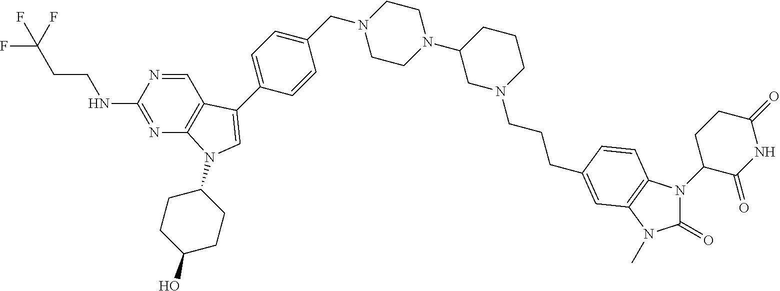 [SMILES: Cn1c(=O)n(C2CCC(=O)NC2=O)c2ccc(CCCN3CCCC(N4CCN(Cc5ccc(-c6cn([C@H]7CC[C@H](O)CC7)c7nc(NCCC(F)(F)F)ncc67)cc5)CC4)C3)cc21] |
| I-119 | 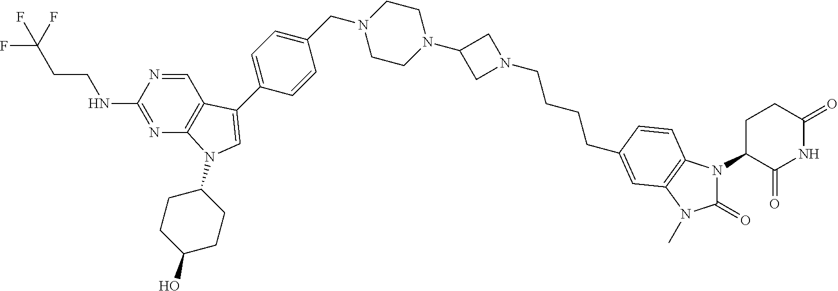 [SMILES: Cn1c(=O)n([C@H]2CCC(=O)NC2=O)c2ccc(CCCCN3CC(N4CCN(Cc5ccc(-c6cn([C@H]7CC[C@H](O)CC7)c7nc(NCCC(F)(F)F)ncc67)cc5)CC4)C3)cc21] |
| I-120 | 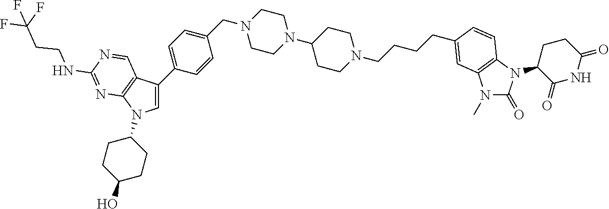 [SMILES: Cn1c(=O)n([C@H]2CCC(=O)NC2=O)c2ccc(CCCCN3CCC(N4CCN(Cc5ccc(-c6cn([C@H]7CC[C@H](O)CC7)c7nc(NCCC(F)(F)F)ncc67)cc5)CC4)CC3)cc21] |
| I-121 | 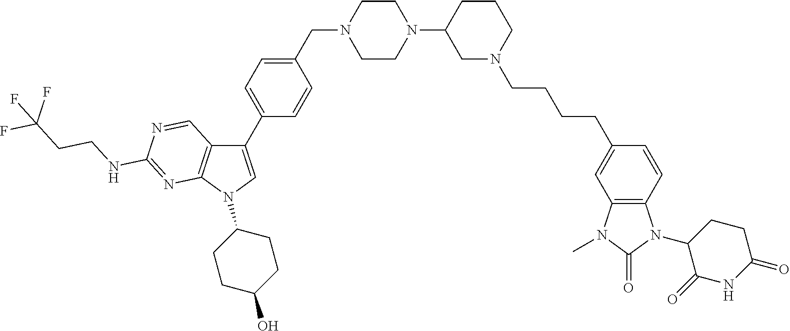 [SMILES: Cn1c(=O)n(C2CCC(=O)NC2=O)c2ccc(CCCCN3CCCC(N4CCN(Cc5ccc(-c6cn([C@H]7CC[C@H](O)CC7)c7nc(NCCC(F)(F)F)ncc67)cc5)CC4)C3)cc21] |
| I-122 | 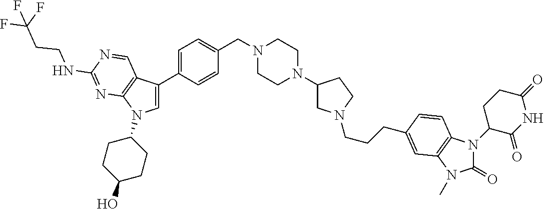 [SMILES: Cn1c(=O)n(C2CCC(=O)NC2=O)c2ccc(CCCN3CCC(N4CCN(Cc5ccc(-c6cn([C@H]7CC[C@H](O)CC7)c7nc(NCCC(F)(F)F)ncc67)cc5)CC4)C3)cc21] |
| I-123 | 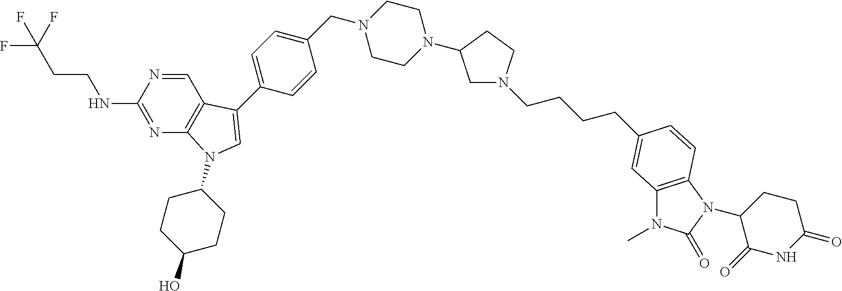 [SMILES: Cn1c(=O)n(C2CCC(=O)NC2=O)c2ccc(CCCCN3CCC(N4CCN(Cc5ccc(-c6cn([C@H]7CC[C@H](O)CC7)c7nc(NCCC(F)(F)F)ncc67)cc5)CC4)C3)cc21] |
| I-124 | 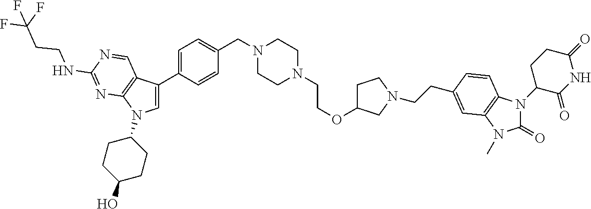 [SMILES: Cn1c(=O)n(C2CCC(=O)NC2=O)c2ccc(CCN3CCC(OCCN4CCN(Cc5ccc(-c6cn([C@H]7CC[C@H](O)CC7)c7nc(NCCC(F)(F)F)ncc67)cc5)CC4)C3)cc21] |
| I-125 | 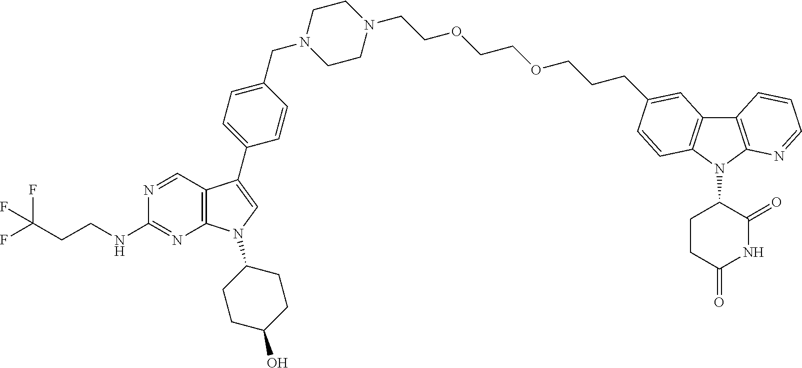 [SMILES: O=C1CC[C@H](n2c3ccc(CCCOCCOCCN4CCN(Cc5ccc(-c6cn([C@H]7CC[C@H](O)CC7)c7nc(NCCC(F)(F)F)ncc67)cc5)CC4)cc3c3cccnc32)C(=O)N1] |
| I-126 | 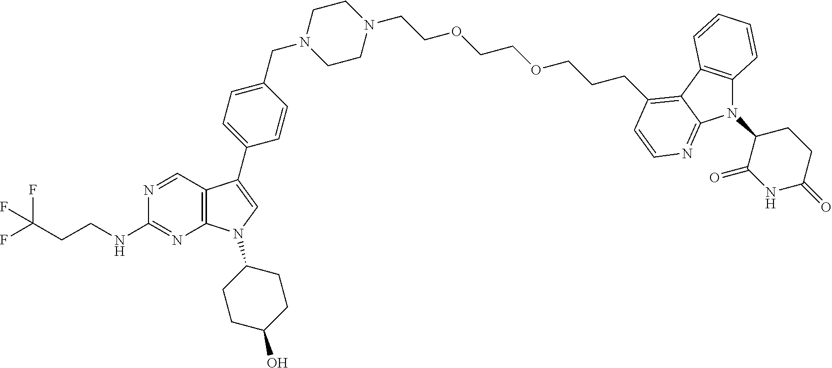 [SMILES: O=C1CC[C@H](n2c3ccccc3c3c(CCCOCCOCCN4CCN(Cc5ccc(-c6cn([C@H]7CC[C@H](O)CC7)c7nc(NCCC(F)(F)F)ncc67)cc5)CC4)ccnc32)C(=O)N1] |
| I-127 | 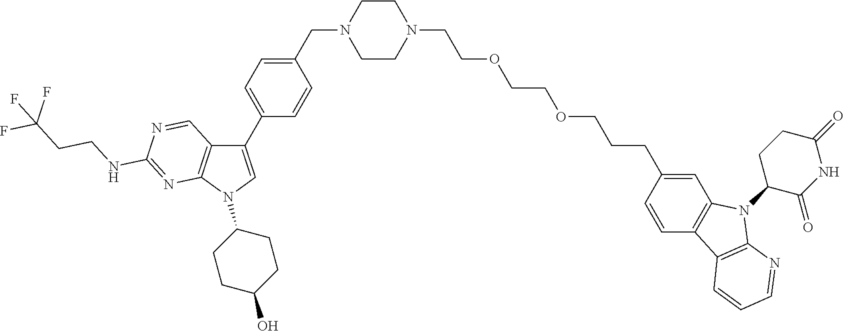 [SMILES: O=C1CC[C@H](n2c3cc(CCCOCCOCCN4CCN(Cc5ccc(-c6cn([C@H]7CC[C@H](O)CC7)c7nc(NCCC(F)(F)F)ncc67)cc5)CC4)ccc3c3cccnc32)C(=O)N1] |
| I-128 | 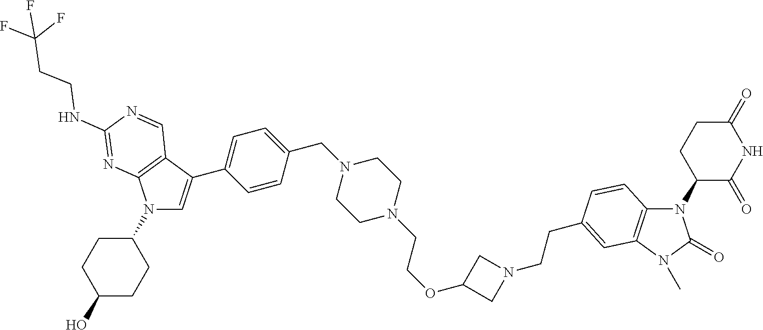 [SMILES: Cn1c(=O)n([C@H]2CCC(=O)NC2=O)c2ccc(CCN3CC(OCCN4CCN(Cc5ccc(-c6cn([C@H]7CC[C@H](O)CC7)c7nc(NCCC(F)(F)F)ncc67)cc5)CC4)C3)cc21] |
| I-129 | 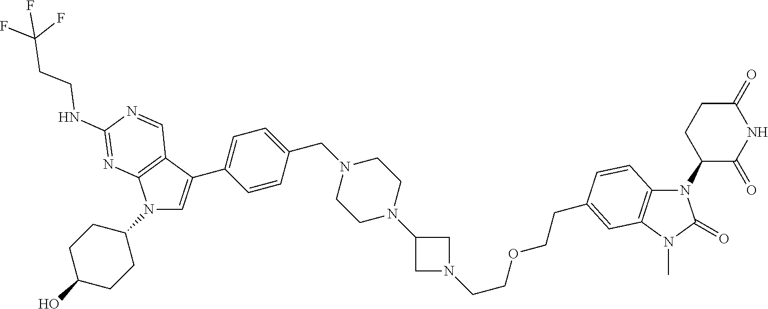 [SMILES: Cn1c(=O)n([C@H]2CCC(=O)NC2=O)c2ccc(CCOCCN3CC(N4CCN(Cc5ccc(-c6cn([C@H]7CC[C@H](O)CC7)c7nc(NCCC(F)(F)F)ncc67)cc5)CC4)C3)cc21] |
| I-130 | 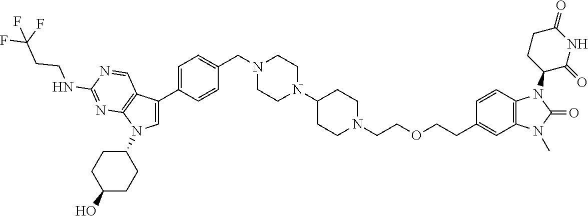 [SMILES: Cn1c(=O)n([C@H]2CCC(=O)NC2=O)c2ccc(CCOCCN3CCC(N4CCN(Cc5ccc(-c6cn([C@H]7CC[C@H](O)CC7)c7nc(NCCC(F)(F)F)ncc67)cc5)CC4)CC3)cc21] |
| I-131 | 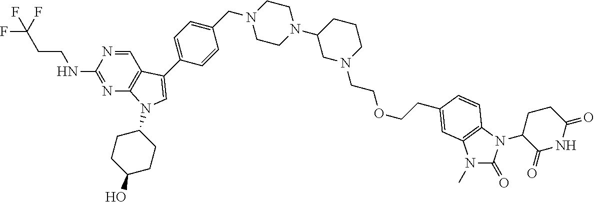 [SMILES: Cn1c(=O)n(C2CCC(=O)NC2=O)c2ccc(CCOCCN3CCCC(N4CCN(Cc5ccc(-c6cn([C@H]7CC[C@H](O)CC7)c7nc(NCCC(F)(F)F)ncc67)cc5)CC4)C3)cc21] |
| I-132 | 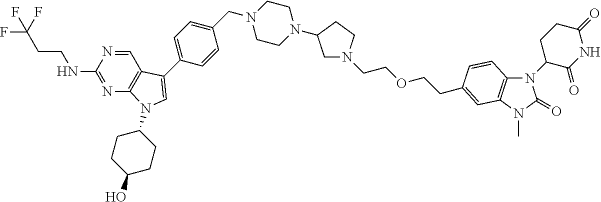 [SMILES: Cn1c(=O)n(C2CCC(=O)NC2=O)c2ccc(CCOCCN3CCC(N4CCN(Cc5ccc(-c6cn([C@H]7CC[C@H](O)CC7)c7nc(NCCC(F)(F)F)ncc67)cc5)CC4)C3)cc21] |
| I-133 | 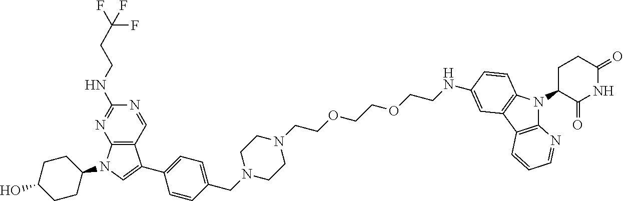 [SMILES: O=C1CC[C@H](n2c3ccc(NCCOCCOCCN4CCN(Cc5ccc(-c6cn([C@H]7CC[C@H](O)CC7)c7nc(NCCC(F)(F)F)ncc67)cc5)CC4)cc3c3cccnc32)C(=O)N1] |
| I-134 | 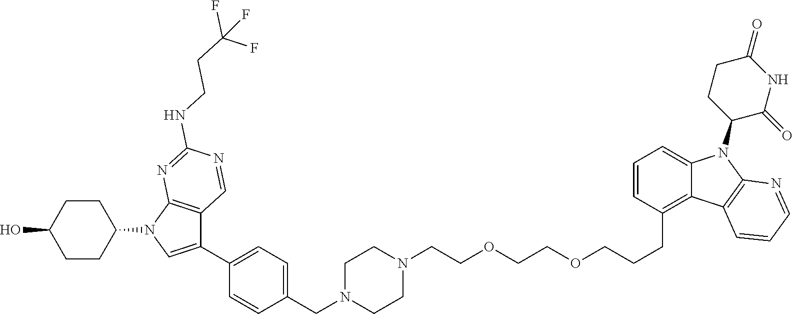 [SMILES: O=C1CC[C@H](n2c3cccc(CCCOCCOCCN4CCN(Cc5ccc(-c6cn([C@H]7CC[C@H](O)CC7)c7nc(NCCC(F)(F)F)ncc67)cc5)CC4)c3c3cccnc32)C(=O)N1] |
| I-135 | 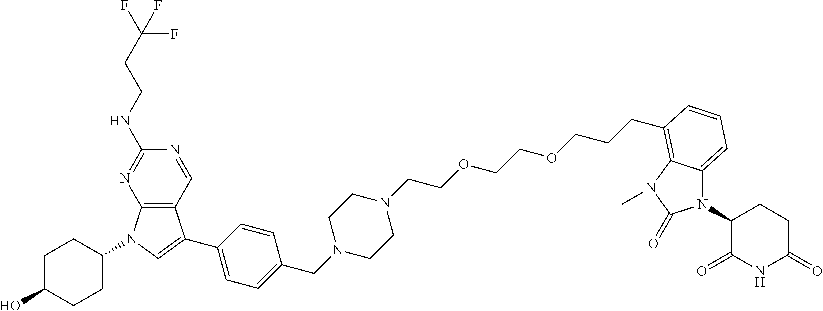 [SMILES: Cn1c(=O)n([C@H]2CCC(=O)NC2=O)c2cccc(CCCOCCOCCN3CCN(Cc4ccc(-c5cn([C@H]6CC[C@H](O)CC6)c6nc(NCCC(F)(F)F)ncc56)cc4)CC3)c21] |
| I-136 | 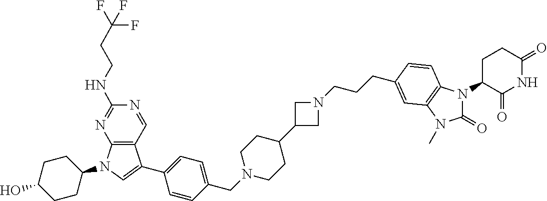 [SMILES: Cn1c(=O)n([C@H]2CCC(=O)NC2=O)c2ccc(CCCN3CC(C4CCN(Cc5ccc(-c6cn([C@H]7CC[C@H](O)CC7)c7nc(NCCC(F)(F)F)ncc67)cc5)CC4)C3)cc21] |
| I-137 | 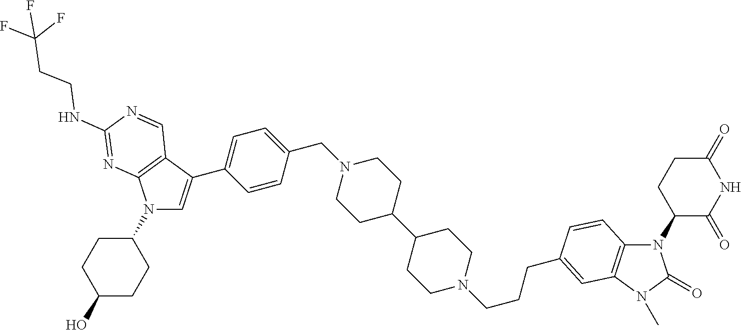 [SMILES: Cn1c(=O)n([C@H]2CCC(=O)NC2=O)c2ccc(CCCN3CCC(C4CCN(Cc5ccc(-c6cn([C@H]7CC[C@H](O)CC7)c7nc(NCCC(F)(F)F)ncc67)cc5)CC4)CC3)cc21] |
| I-138 | 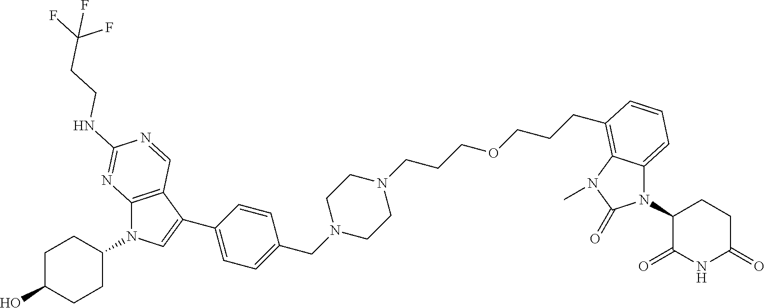 [SMILES: Cn1c(=O)n([C@H]2CCC(=O)NC2=O)c2cccc(CCCOCCCN3CCN(Cc4ccc(-c5cn([C@H]6CC[C@H](O)CC6)c6nc(NCCC(F)(F)F)ncc56)cc4)CC3)c21] |
| I-139 | 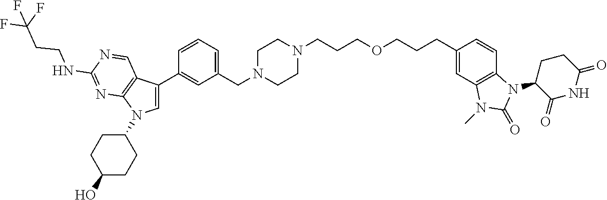 [SMILES: Cn1c(=O)n([C@H]2CCC(=O)NC2=O)c2ccc(CCCOCCCN3CCN(Cc4cccc(-c5cn([C@H]6CC[C@H](O)CC6)c6nc(NCCC(F)(F)F)ncc56)c4)CC3)cc21] |
| I-140 | 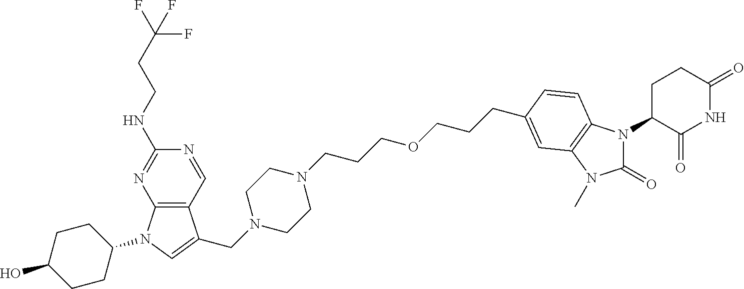 [SMILES: Cn1c(=O)n([C@H]2CCC(=O)NC2=O)c2ccc(CCCOCCCN3CCN(Cc4cn([C@H]5CC[C@H](O)CC5)c5nc(NCCC(F)(F)F)ncc45)CC3)cc21] |
| I-141 | 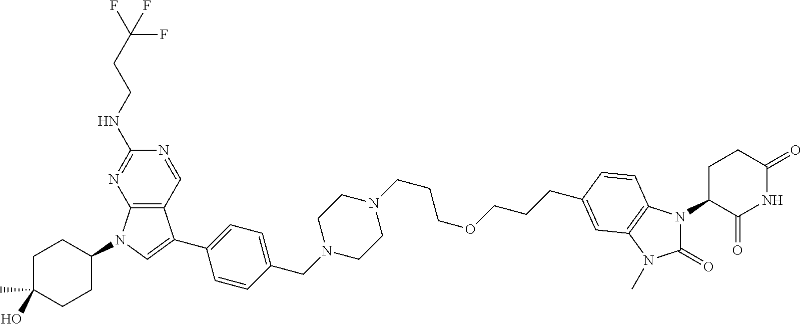 [SMILES: Cn1c(=O)n([C@H]2CCC(=O)NC2=O)c2ccc(CCCOCCCN3CCN(Cc4ccc(-c5cn([C@H]6CC[C@@](C)(O)CC6)c6nc(NCCC(F)(F)F)ncc56)cc4)CC3)cc21] |
| I-142 | 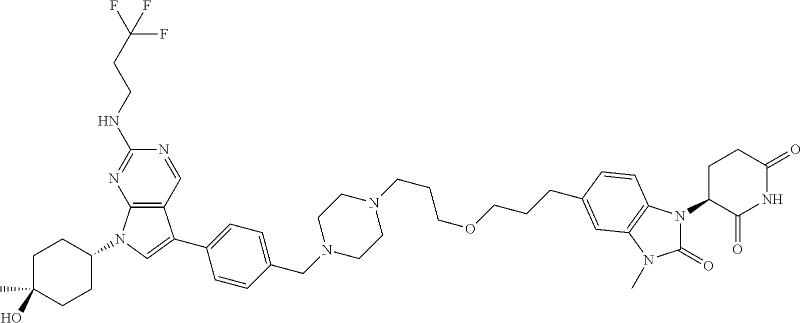 [SMILES: Cn1c(=O)n([C@H]2CCC(=O)NC2=O)c2ccc(CCCOCCCN3CCN(Cc4ccc(-c5cn([C@H]6CC[C@](C)(O)CC6)c6nc(NCCC(F)(F)F)ncc56)cc4)CC3)cc21] |
| I-143 | 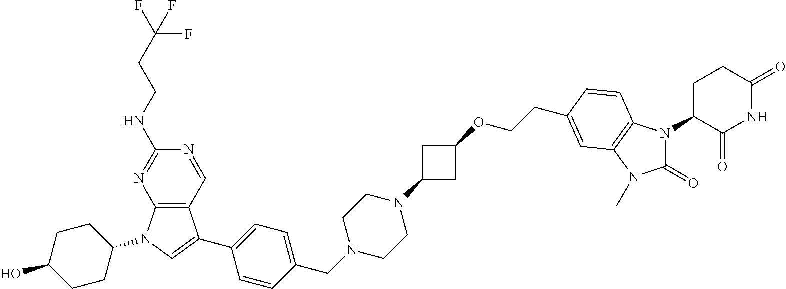 [SMILES: Cn1c(=O)n([C@H]2CCC(=O)NC2=O)c2ccc(CCO[C@H]3C[C@@H](N4CCN(Cc5ccc(-c6cn([C@H]7CC[C@H](O)CC7)c7nc(NCCC(F)(F)F)ncc67)cc5)CC4)C3)cc21] |
| I-144 | 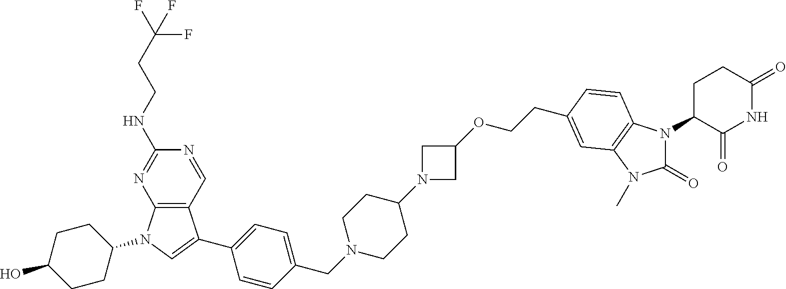 [SMILES: Cn1c(=O)n([C@H]2CCC(=O)NC2=O)c2ccc(CCOC3CN(C4CCN(Cc5ccc(-c6cn([C@H]7CC[C@H](O)CC7)c7nc(NCCC(F)(F)F)ncc67)cc5)CC4)C3)cc21] |
| I-145 | 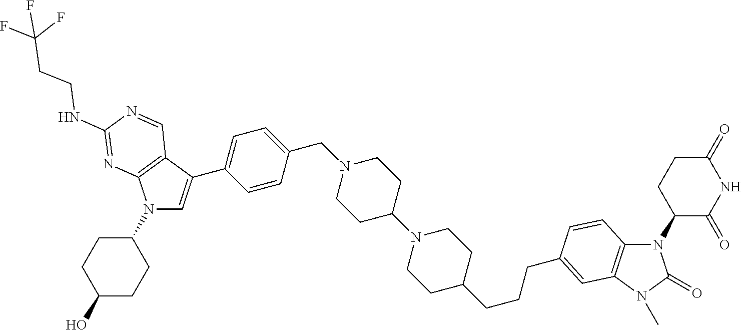 [SMILES: Cn1c(=O)n([C@H]2CCC(=O)NC2=O)c2ccc(CCCC3CCN(C4CCN(Cc5ccc(-c6cn([C@H]7CC[C@H](O)CC7)c7nc(NCCC(F)(F)F)ncc67)cc5)CC4)CC3)cc21] |
| I-146 | 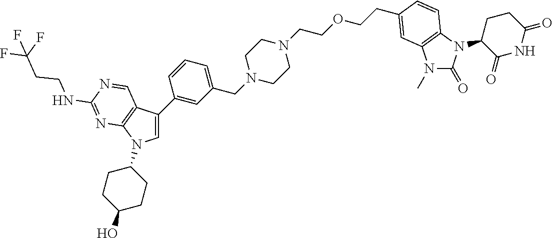 [SMILES: Cn1c(=O)n([C@H]2CCC(=O)NC2=O)c2ccc(CCOCCN3CCN(Cc4cccc(-c5cn([C@H]6CC[C@H](O)CC6)c6nc(NCCC(F)(F)F)ncc56)c4)CC3)cc21] |
| I-147 | 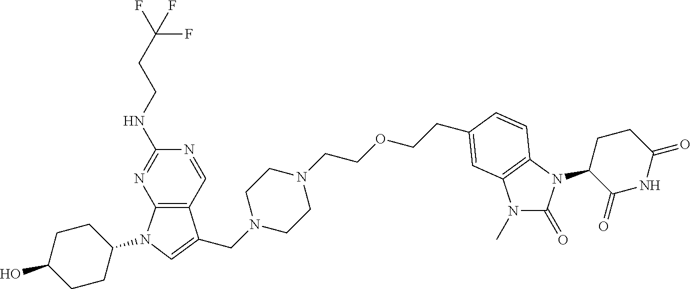 [SMILES: Cn1c(=O)n([C@H]2CCC(=O)NC2=O)c2ccc(CCOCCN3CCN(Cc4cn([C@H]5CC[C@H](O)CC5)c5nc(NCCC(F)(F)F)ncc45)CC3)cc21] |
| I-148 | 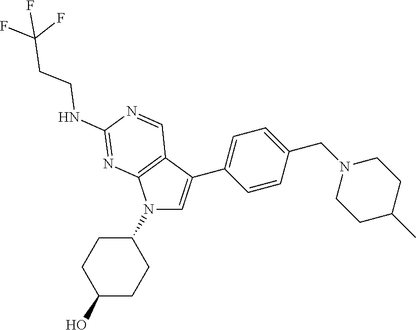 [SMILES: CC1CCN(Cc2ccc(-c3cn([C@H]4CC[C@H](O)CC4)c4nc(NCCC(F)(F)F)ncc34)cc2)CC1] |
| I-149 | 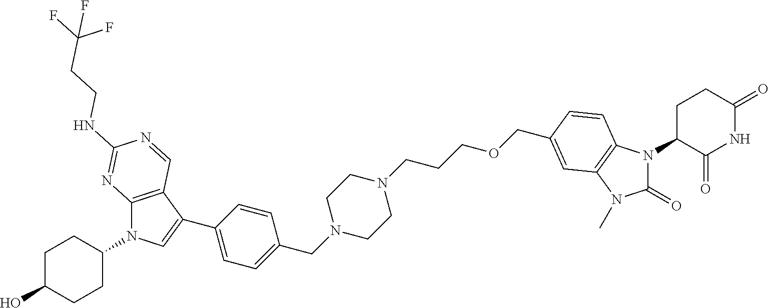 [SMILES: Cn1c(=O)n([C@H]2CCC(=O)NC2=O)c2ccc(COCCCN3CCN(Cc4ccc(-c5cn([C@H]6CC[C@H](O)CC6)c6nc(NCCC(F)(F)F)ncc56)cc4)CC3)cc21] |
| I-150 | 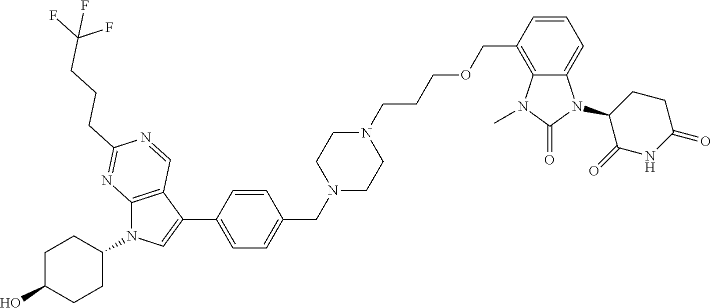 [SMILES: Cn1c(=O)n([C@H]2CCC(=O)NC2=O)c2cccc(COCCCN3CCN(Cc4ccc(-c5cn([C@H]6CC[C@H](O)CC6)c6nc(CCCC(F)(F)F)ncc56)cc4)CC3)c21] |
| I-151 | 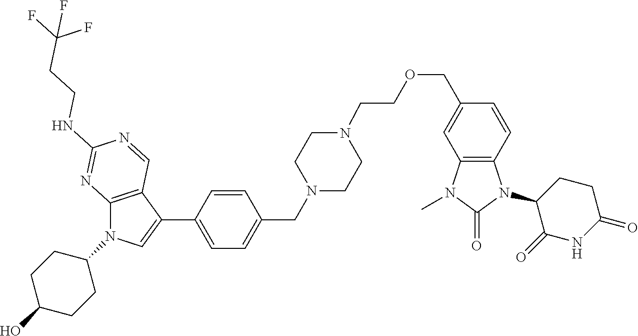 [SMILES: Cn1c(=O)n([C@H]2CCC(=O)NC2=O)c2ccc(COCCN3CCN(Cc4ccc(-c5cn([C@H]6CC[C@H](O)CC6)c6nc(NCCC(F)(F)F)ncc56)cc4)CC3)cc21] |
| I-152 | 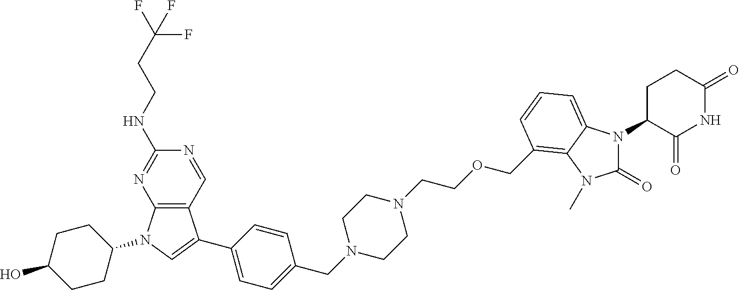 [SMILES: Cn1c(=O)n([C@H]2CCC(=O)NC2=O)c2cccc(COCCN3CCN(Cc4ccc(-c5cn([C@H]6CC[C@H](O)CC6)c6nc(NCCC(F)(F)F)ncc56)cc4)CC3)c21] |
| I-153 | 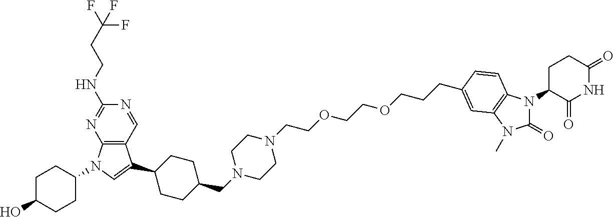 [SMILES: Cn1c(=O)n([C@H]2CCC(=O)NC2=O)c2ccc(CCCOCCOCCN3CCN(C[C@H]4CC[C@@H](c5cn([C@H]6CC[C@H](O)CC6)c6nc(NCCC(F)(F)F)ncc65)CC4)CC3)cc21] |
| I-154 | 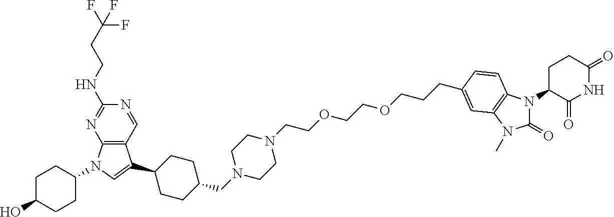 [SMILES: Cn1c(=O)n([C@H]2CCC(=O)NC2=O)c2ccc(CCCOCCOCCN3CCN(C[C@H]4CC[C@H](c5cn([C@H]6CC[C@H](O)CC6)c6nc(NCCC(F)(F)F)ncc65)CC4)CC3)cc21] |
| I-155 | 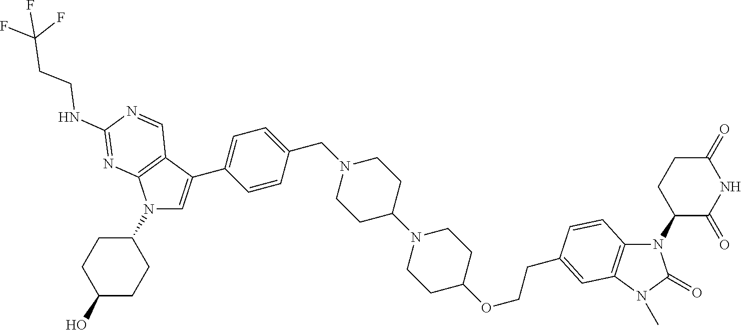 [SMILES: Cn1c(=O)n([C@H]2CCC(=O)NC2=O)c2ccc(CCOC3CCN(C4CCN(Cc5ccc(-c6cn([C@H]7CC[C@H](O)CC7)c7nc(NCCC(F)(F)F)ncc67)cc5)CC4)CC3)cc21] |
| I-156 | 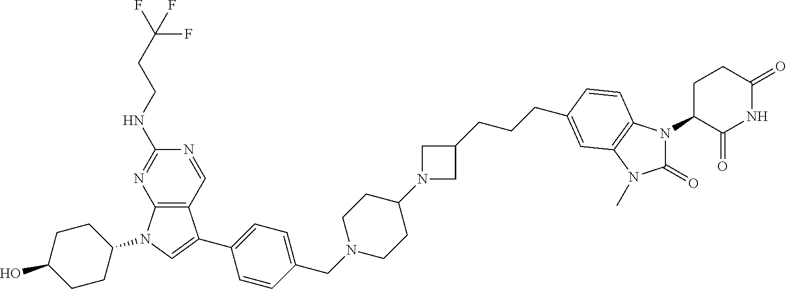 [SMILES: Cn1c(=O)n([C@H]2CCC(=O)NC2=O)c2ccc(CCCC3CN(C4CCN(Cc5ccc(-c6cn([C@H]7CC[C@H](O)CC7)c7nc(NCCC(F)(F)F)ncc67)cc5)CC4)C3)cc21] |
| I-157 | 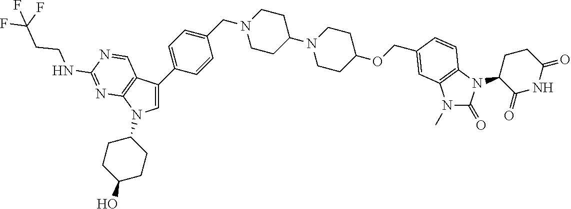 [SMILES: Cn1c(=O)n([C@H]2CCC(=O)NC2=O)c2ccc(COC3CCN(C4CCN(Cc5ccc(-c6cn([C@H]7CC[C@H](O)CC7)c7nc(NCCC(F)(F)F)ncc67)cc5)CC4)CC3)cc21] |
| I-158 | 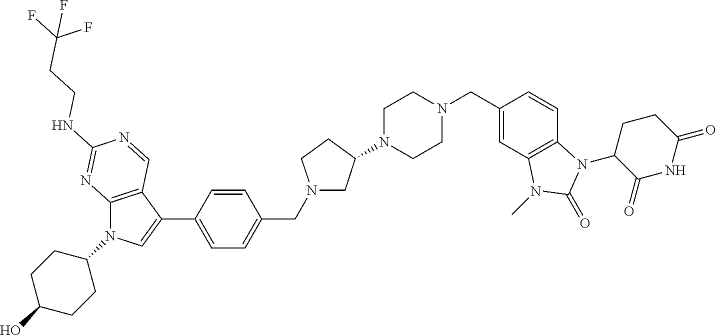 [SMILES: Cn1c(=O)n(C2CCC(=O)NC2=O)c2ccc(CN3CCN([C@H]4CCN(Cc5ccc(-c6cn([C@H]7CC[C@H](O)CC7)c7nc(NCCC(F)(F)F)ncc67)cc5)C4)CC3)cc21] |
| I-159 | 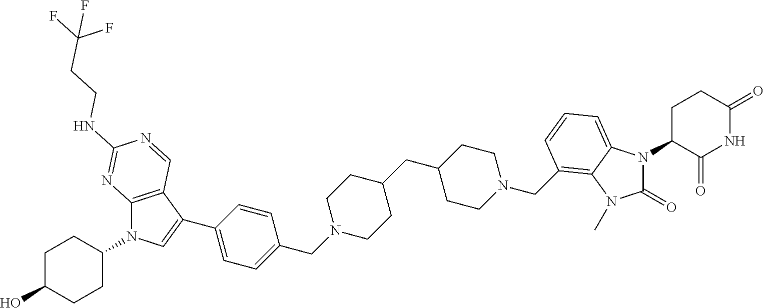 [SMILES: Cn1c(=O)n([C@H]2CCC(=O)NC2=O)c2cccc(CN3CCC(CC4CCN(Cc5ccc(-c6cn([C@H]7CC[C@H](O)CC7)c7nc(NCCC(F)(F)F)ncc67)cc5)CC4)CC3)c21] |
| I-160 | 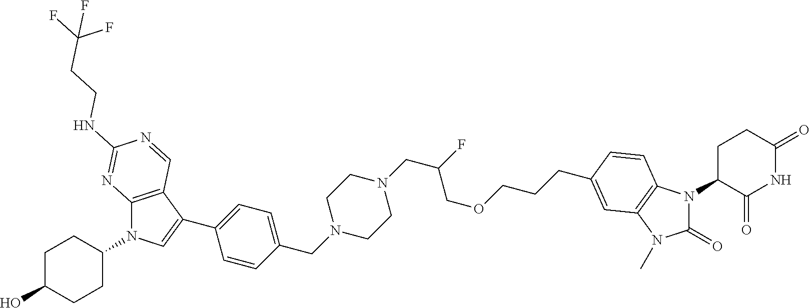 [SMILES: Cn1c(=O)n([C@H]2CCC(=O)NC2=O)c2ccc(CCCOCC(F)CN3CCN(Cc4ccc(-c5cn([C@H]6CC[C@H](O)CC6)c6nc(NCCC(F)(F)F)ncc56)cc4)CC3)cc21] |
| I-161 | 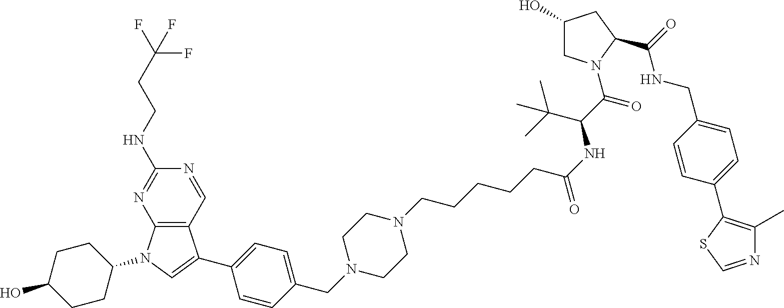 [SMILES: Cc1ncsc1-c1ccc(CNC(=O)[C@@H]2C[C@@H](O)CN2C(=O)[C@@H](NC(=O)CCCCCN2CCN(Cc3ccc(-c4cn([C@H]5CC[C@H](O)CC5)c5nc(NCCC(F)(F)F)ncc45)cc3)CC2)C(C)(C)C)cc1] |
| I-162 | 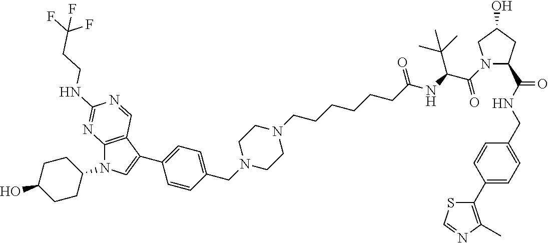 [SMILES: Cc1ncsc1-c1ccc(CNC(=O)[C@@H]2C[C@@H](O)CN2C(=O)[C@@H](NC(=O)CCCCCCN2CCN(Cc3ccc(-c4cn([C@H]5CC[C@H](O)CC5)c5nc(NCCC(F)(F)F)ncc45)cc3)CC2)C(C)(C)C)cc1] |
| I-163 | 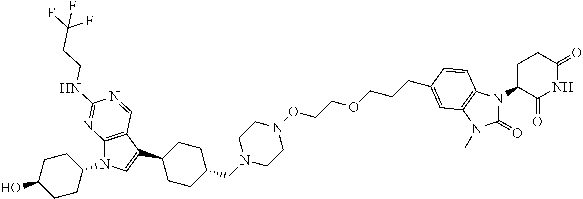 [SMILES: Cn1c(=O)n([C@H]2CCC(=O)NC2=O)c2ccc(CCCOCCON3CCN(C[C@H]4CC[C@H](c5cn([C@H]6CC[C@H](O)CC6)c6nc(NCCC(F)(F)F)ncc65)CC4)CC3)cc21] |
| I-164 | 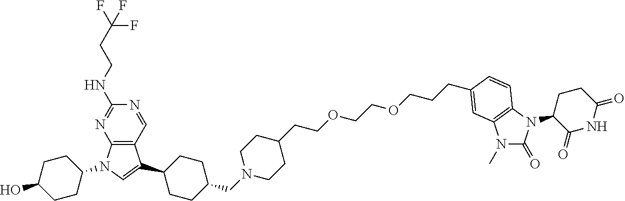 [SMILES: Cn1c(=O)n([C@H]2CCC(=O)NC2=O)c2ccc(CCCOCCOCCC3CCN(C[C@H]4CC[C@H](c5cn([C@H]6CC[C@H](O)CC6)c6nc(NCCC(F)(F)F)ncc65)CC4)CC3)cc21] |
| I-165 | 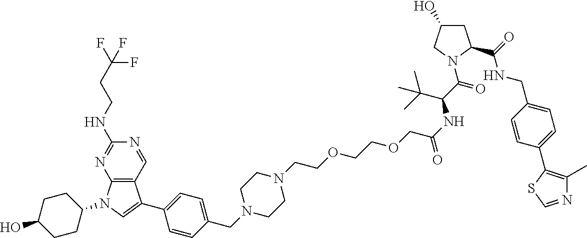 [SMILES: Cc1ncsc1-c1ccc(CNC(=O)[C@@H]2C[C@@H](O)CN2C(=O)[C@@H](NC(=O)COCCOCCN2CCN(Cc3ccc(-c4cn([C@H]5CC[C@H](O)CC5)c5nc(NCCC(F)(F)F)ncc45)cc3)CC2)C(C)(C)C)cc1] |
| I-166 | 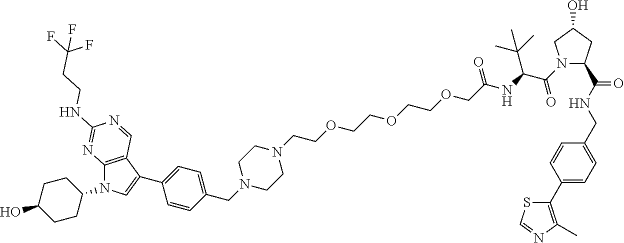 [SMILES: Cc1ncsc1-c1ccc(CNC(=O)[C@@H]2C[C@@H](O)CN2C(=O)[C@@H](NC(=O)COCCOCCOCCN2CCN(Cc3ccc(-c4cn([C@H]5CC[C@H](O)CC5)c5nc(NCCC(F)(F)F)ncc45)cc3)CC2)C(C)(C)C)cc1] |
| I-167 | 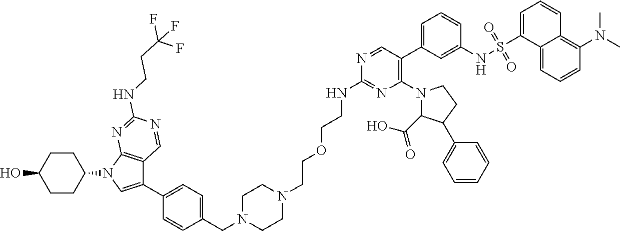 [SMILES: CN(C)c1cccc2c(S(=O)(=O)Nc3cccc(-c4cnc(NCCOCCN5CCN(Cc6ccc(-c7cn([C@H]8CC[C@H](O)CC8)c8nc(NCCC(F)(F)F)ncc78)cc6)CC5)nc4N4CCC(c5ccccc5)C4C(=O)O)c3)cccc12] |
| I-168 | 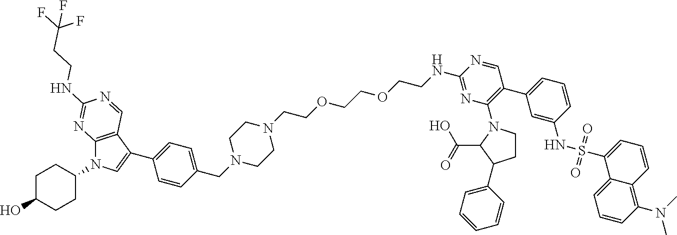 [SMILES: CN(C)c1cccc2c(S(=O)(=O)Nc3cccc(-c4cnc(NCCOCCOCCN5CCN(Cc6ccc(-c7cn([C@H]8CC[C@H](O)CC8)c8nc(NCCC(F)(F)F)ncc78)cc6)CC5)nc4N4CCC(c5ccccc5)C4C(=O)O)c3)cccc12] |
| I-169 | 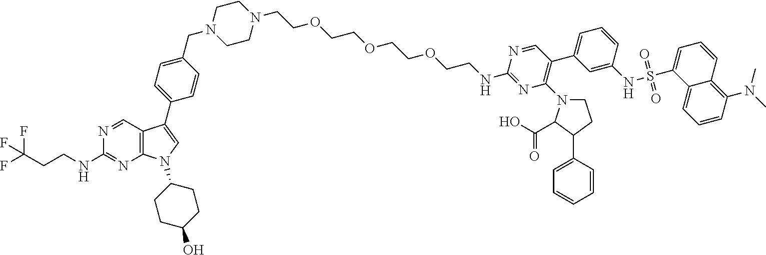 [SMILES: CN(C)c1cccc2c(S(=O)(=O)Nc3cccc(-c4cnc(NCCOCCOCCOCCN5CCN(Cc6ccc(-c7cn([C@H]8CC[C@H](O)CC8)c8nc(NCCC(F)(F)F)ncc78)cc6)CC5)nc4N4CCC(c5ccccc5)C4C(=O)O)c3)cccc12] |
| I-170 | 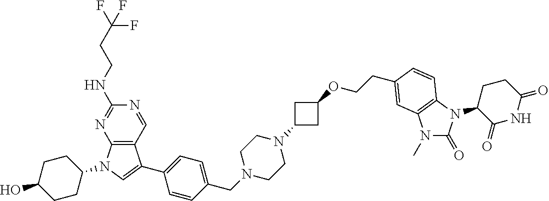 [SMILES: Cn1c(=O)n([C@H]2CCC(=O)NC2=O)c2ccc(CCO[C@H]3C[C@H](N4CCN(Cc5ccc(-c6cn([C@H]7CC[C@H](O)CC7)c7nc(NCCC(F)(F)F)ncc67)cc5)CC4)C3)cc21] |
| I-171 | 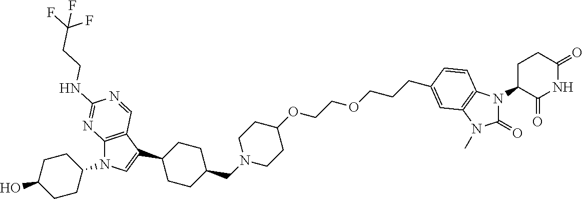 [SMILES: Cn1c(=O)n([C@H]2CCC(=O)NC2=O)c2ccc(CCCOCCOC3CCN(C[C@H]4CC[C@@H](c5cn([C@H]6CC[C@H](O)CC6)c6nc(NCCC(F)(F)F)ncc65)CC4)CC3)cc21] |
| I-172 | 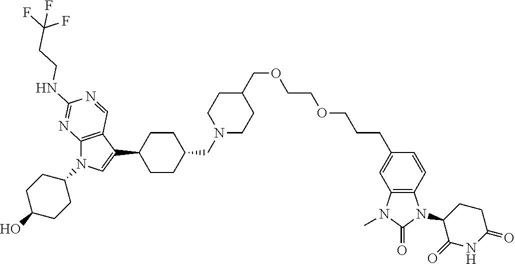 [SMILES: Cn1c(=O)n([C@H]2CCC(=O)NC2=O)c2ccc(CCCOCCOCC3CCN(C[C@H]4CC[C@H](c5cn([C@H]6CC[C@H](O)CC6)c6nc(NCCC(F)(F)F)ncc65)CC4)CC3)cc21] |
| I-173 | 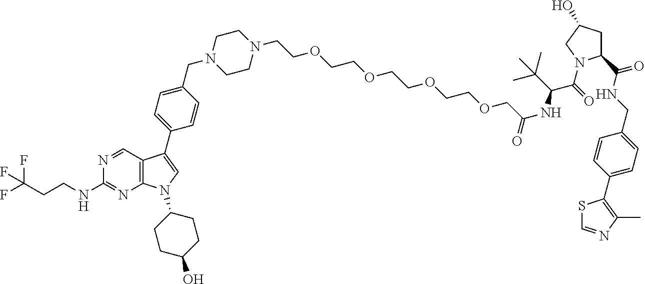 [SMILES: Cc1ncsc1-c1ccc(CNC(=O)[C@@H]2C[C@@H](O)CN2C(=O)[C@@H](NC(=O)COCCOCCOCCOCCN2CCN(Cc3ccc(-c4cn([C@H]5CC[C@H](O)CC5)c5nc(NCCC(F)(F)F)ncc45)cc3)CC2)C(C)(C)C)cc1] |
| I-174 | 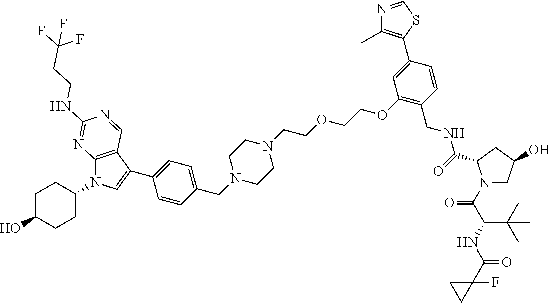 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCN2CCN(Cc3ccc(-c4cn([C@H]5CC[C@H](O)CC5)c5nc(NCCC(F)(F)F)ncc45)cc3)CC2)c1] |
| I-175 | 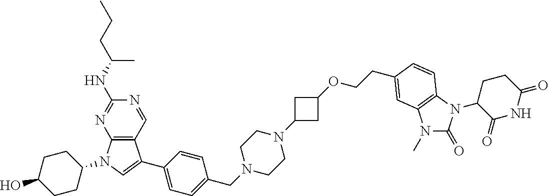 [SMILES: CCC[C@H](C)Nc1ncc2c(-c3ccc(CN4CCN(C5CC(OCCc6ccc7c(c6)n(C)c(=O)n7C6CCC(=O)NC6=O)C5)CC4)cc3)cn([C@H]3CC[C@H](O)CC3)c2n1] |
| I-176 | 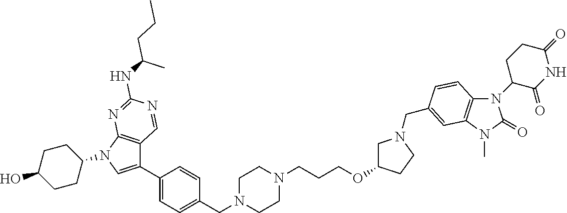 [SMILES: CCC[C@@H](C)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-177 | 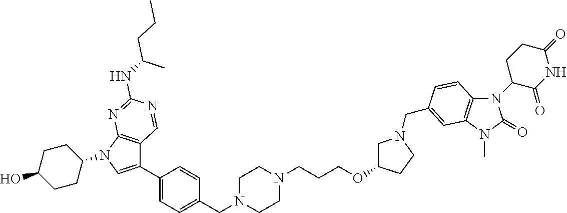 [SMILES: CCC[C@H](C)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-178 | 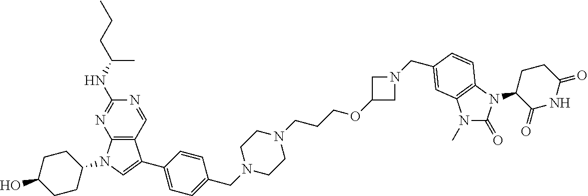 [SMILES: CCC[C@H](C)Nc1ncc2c(-c3ccc(CN4CCN(CCCOC5CN(Cc6ccc7c(c6)n(C)c(=O)n7[C@H]6CCC(=O)NC6=O)C5)CC4)cc3)cn([C@H]3CC[C@H](O)CC3)c2n1] |
| I-179 | 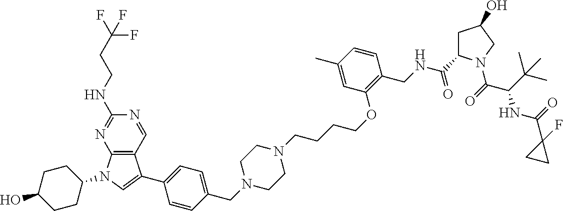 [SMILES: Cc1ccc(CNC(=O)[C@@H]2C[C@@H](O)CN2C(=O)[C@@H](NC(=O)C2(F)CC2)C(C)(C)C)c(OCCCCN2CCN(Cc3ccc(-c4cn([C@H]5CC[C@H](O)CC5)c5nc(NCCC(F)(F)F)ncc45)cc3)CC2)c1] |
| I-180 | 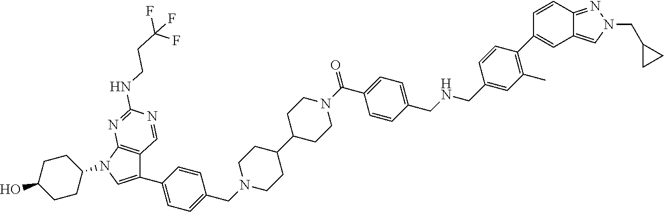 [SMILES: Cc1cc(CNCc2ccc(C(=O)N3CCC(C4CCN(Cc5ccc(-c6cn([C@H]7CC[C@H](O)CC7)c7nc(NCCC(F)(F)F)ncc67)cc5)CC4)CC3)cc2)ccc1-c1ccc2nn(CC3CC3)cc2c1] |
| I-181 | 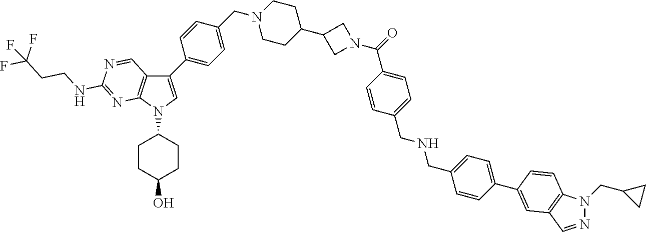 [SMILES: O=C(c1ccc(CNCc2ccc(-c3ccc4c(cnn4CC4CC4)c3)cc2)cc1)N1CC(C2CCN(Cc3ccc(-c4cn([C@H]5CC[C@H](O)CC5)c5nc(NCCC(F)(F)F)ncc45)cc3)CC2)C1] |
| I-182 | 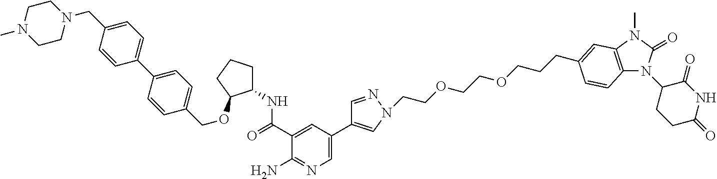 [SMILES: CN1CCN(Cc2ccc(-c3ccc(CO[C@H]4CCC[C@@H]4NC(=O)c4cc(-c5cnn(CCOCCOCCCc6ccc7c(c6)n(C)c(=O)n7C6CCC(=O)NC6=O)c5)cnc4N)cc3)cc2)CC1] |
| I-183 | 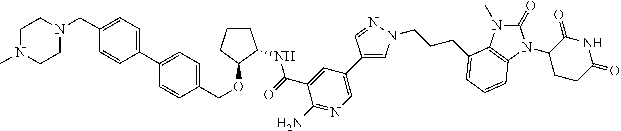 [SMILES: CN1CCN(Cc2ccc(-c3ccc(CO[C@H]4CCC[C@@H]4NC(=O)c4cc(-c5cnn(CCCc6cccc7c6n(C)c(=O)n7C6CCC(=O)NC6=O)c5)cnc4N)cc3)cc2)CC1] |
| I-184 | 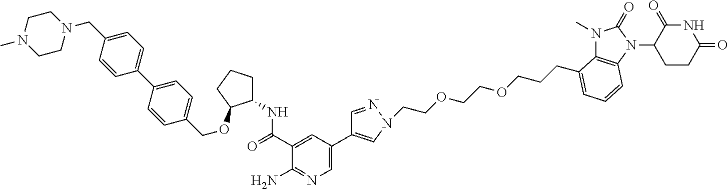 [SMILES: CN1CCN(Cc2ccc(-c3ccc(CO[C@H]4CCC[C@@H]4NC(=O)c4cc(-c5cnn(CCOCCOCCCc6cccc7c6n(C)c(=O)n7C6CCC(=O)NC6=O)c5)cnc4N)cc3)cc2)CC1] |
| I-185 | 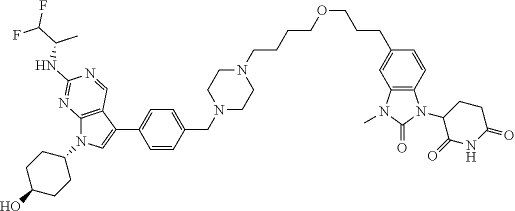 [SMILES: C[C@H](Nc1ncc2c(-c3ccc(CN4CCN(CCCCOCCCc5ccc6c(c5)n(C)c(=O)n6C5CCC(=O)NC5=O)CC4)cc3)cn([C@H]3CC[C@H](O)CC3)c2n1)C(F)F] |
| I-186 | 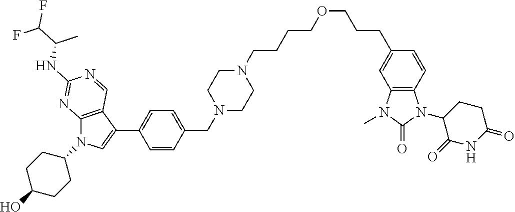 [SMILES: C[C@H](Nc1ncc2c(-c3ccc(CN4CCN(CCCCOCCCc5ccc6c(c5)n(C)c(=O)n6C5CCC(=O)NC5=O)CC4)cc3)cn([C@H]3CC[C@H](O)CC3)c2n1)C(F)F] |
| I-187 | 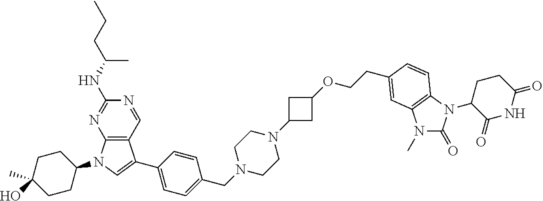 [SMILES: CCC[C@H](C)Nc1ncc2c(-c3ccc(CN4CCN(C5CC(OCCc6ccc7c(c6)n(C)c(=O)n7C6CCC(=O)NC6=O)C5)CC4)cc3)cn([C@H]3CC[C@@](C)(O)CC3)c2n1] |
| I-188 | 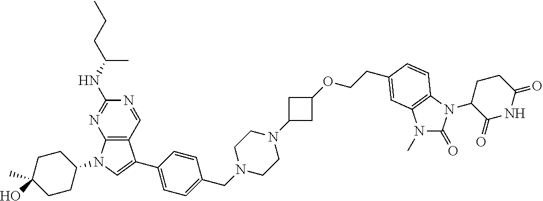 [SMILES: CCC[C@H](C)Nc1ncc2c(-c3ccc(CN4CCN(C5CC(OCCc6ccc7c(c6)n(C)c(=O)n7C6CCC(=O)NC6=O)C5)CC4)cc3)cn([C@H]3CC[C@](C)(O)CC3)c2n1] |
| I-189 | 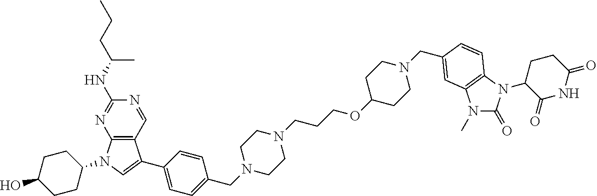 [SMILES: CCC[C@H](C)Nc1ncc2c(-c3ccc(CN4CCN(CCCOC5CCN(Cc6ccc7c(c6)n(C)c(=O)n7C6CCC(=O)NC6=O)CC5)CC4)cc3)cn([C@H]3CC[C@H](O)CC3)c2n1] |
| I-190 | 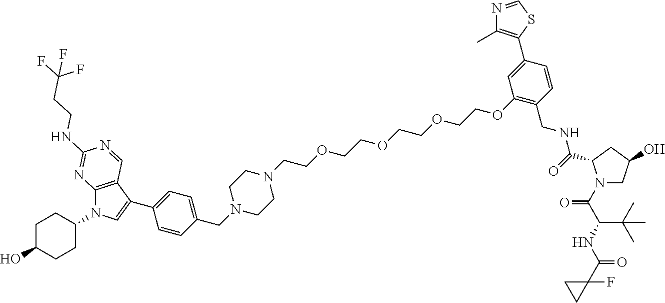 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCN2CCN(Cc3ccc(-c4cn([C@H]5CC[C@H](O)CC5)c5nc(NCCC(F)(F)F)ncc45)cc3)CC2)c1] |
| I-191 | 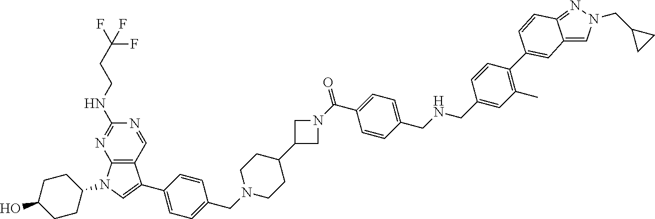 [SMILES: Cc1cc(CNCc2ccc(C(=O)N3CC(C4CCN(Cc5ccc(-c6cn([C@H]7CC[C@H](O)CC7)c7nc(NCCC(F)(F)F)ncc67)cc5)CC4)C3)cc2)ccc1-c1ccc2nn(CC3CC3)cc2c1] |
| I-192 | 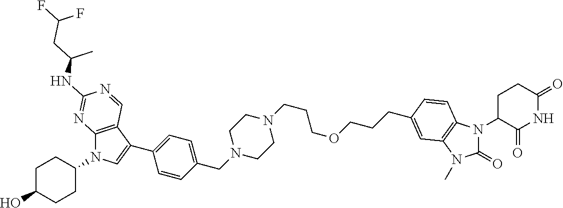 [SMILES: C[C@H](CC(F)F)Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1] |
| I-193 | 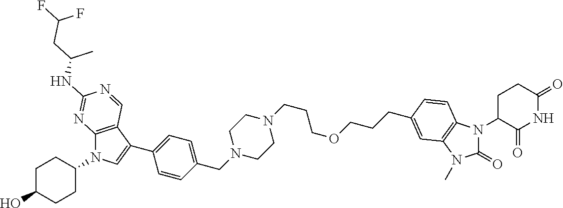 [SMILES: C[C@@H](CC(F)F)Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1] |
| I-194 | 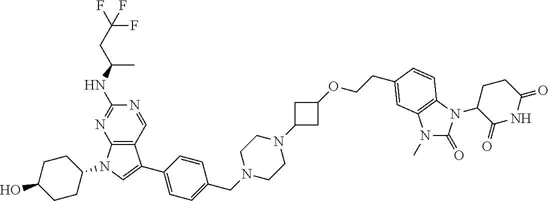 [SMILES: C[C@H](CC(F)(F)F)Nc1ncc2c(-c3ccc(CN4CCN(C5CC(OCCc6ccc7c(c6)n(C)c(=O)n7C6CCC(=O)NC6=O)C5)CC4)cc3)cn([C@H]3CC[C@H](O)CC3)c2n1] |
| I-195 | 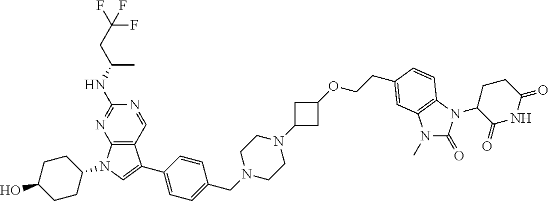 [SMILES: C[C@@H](CC(F)(F)F)Nc1ncc2c(-c3ccc(CN4CCN(C5CC(OCCc6ccc7c(c6)n(C)c(=O)n7C6CCC(=O)NC6=O)C5)CC4)cc3)cn([C@H]3CC[C@H](O)CC3)c2n1] |
| I-196 | 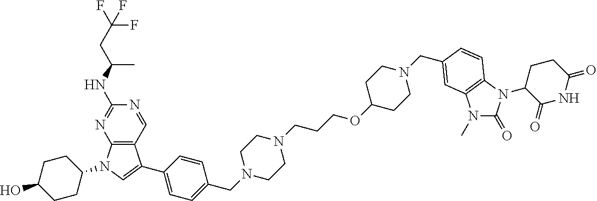 [SMILES: C[C@H](CC(F)(F)F)Nc1ncc2c(-c3ccc(CN4CCN(CCCOC5CCN(Cc6ccc7c(c6)n(C)c(=O)n7C6CCC(=O)NC6=O)CC5)CC4)cc3)cn([C@H]3CC[C@H](O)CC3)c2n1] |
| I-197 | 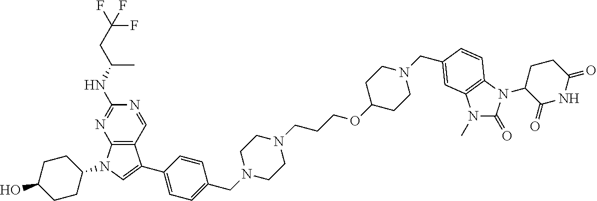 [SMILES: C[C@@H](CC(F)(F)F)Nc1ncc2c(-c3ccc(CN4CCN(CCCOC5CCN(Cc6ccc7c(c6)n(C)c(=O)n7C6CCC(=O)NC6=O)CC5)CC4)cc3)cn([C@H]3CC[C@H](O)CC3)c2n1] |
| I-198 | 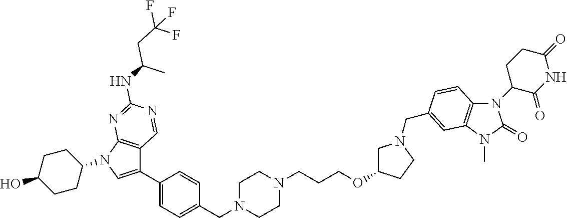 [SMILES: C[C@H](CC(F)(F)F)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-199 | 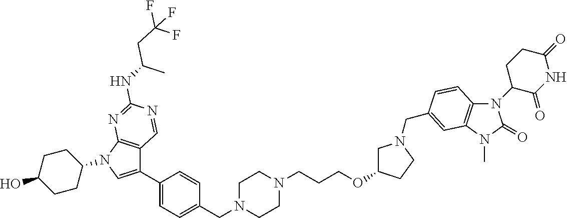 [SMILES: C[C@@H](CC(F)(F)F)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-200 | 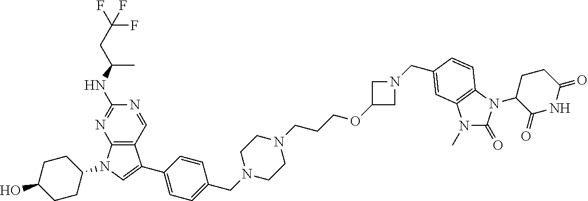 [SMILES: C[C@H](CC(F)(F)F)Nc1ncc2c(-c3ccc(CN4CCN(CCCOC5CN(Cc6ccc7c(c6)n(C)c(=O)n7C6CCC(=O)NC6=O)C5)CC4)cc3)cn([C@H]3CC[C@H](O)CC3)c2n1] |
| I-201 | 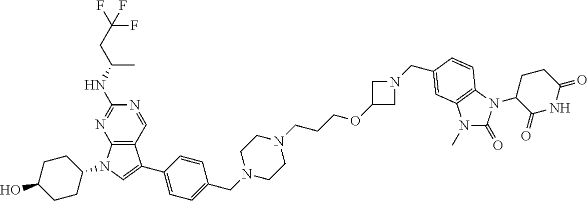 [SMILES: C[C@@H](CC(F)(F)F)Nc1ncc2c(-c3ccc(CN4CCN(CCCOC5CN(Cc6ccc7c(c6)n(C)c(=O)n7C6CCC(=O)NC6=O)C5)CC4)cc3)cn([C@H]3CC[C@H](O)CC3)c2n1] |
| I-202 | 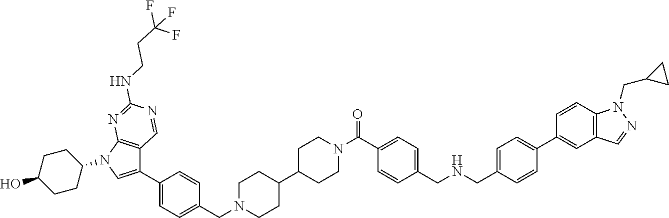 [SMILES: O=C(c1ccc(CNCc2ccc(-c3ccc4c(cnn4CC4CC4)c3)cc2)cc1)N1CCC(C2CCN(Cc3ccc(-c4cn([C@H]5CC[C@H](O)CC5)c5nc(NCCC(F)(F)F)ncc45)cc3)CC2)CC1] |
| I-203 | 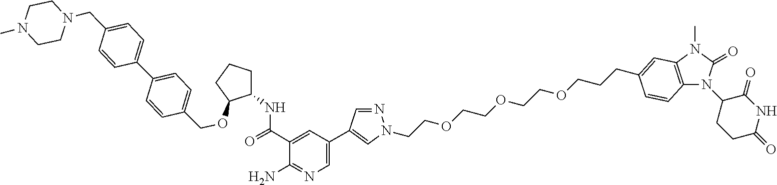 [SMILES: CN1CCN(Cc2ccc(-c3ccc(CO[C@H]4CCC[C@@H]4NC(=O)c4cc(-c5cnn(CCOCCOCCOCCCc6ccc7c(c6)n(C)c(=O)n7C6CCC(=O)NC6=O)c5)cnc4N)cc3)cc2)CC1] |
| I-204 | 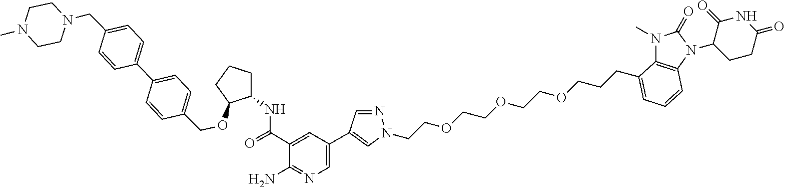 [SMILES: CN1CCN(Cc2ccc(-c3ccc(CO[C@H]4CCC[C@@H]4NC(=O)c4cc(-c5cnn(CCOCCOCCOCCCc6cccc7c6n(C)c(=O)n7C6CCC(=O)NC6=O)c5)cnc4N)cc3)cc2)CC1] |
| I-205 | 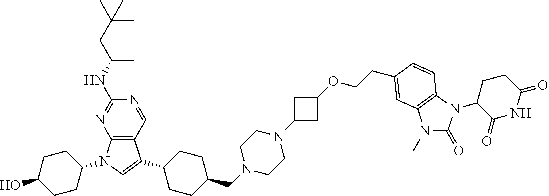 [SMILES: C[C@@H](CC(C)(C)C)Nc1ncc2c(n1)n([C@H]1CC[C@H](O)CC1)cc2[C@H]1CC[C@H](CN2CCN(C3CC(OCCc4ccc5c(c4)n(C)c(=O)n5C4CCC(=O)NC4=O)C3)CC2)CC1] |
| I-206 | 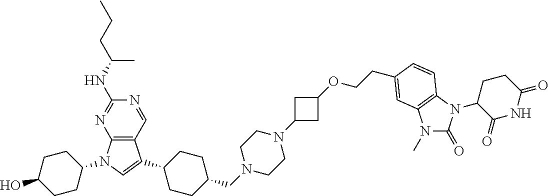 [SMILES: CCC[C@H](C)Nc1ncc2c(n1)n([C@H]1CC[C@H](O)CC1)cc2[C@H]1CC[C@@H](CN2CCN(C3CC(OCCc4ccc5c(c4)n(C)c(=O)n5C4CCC(=O)NC4=O)C3)CC2)CC1] |
| I-207 | 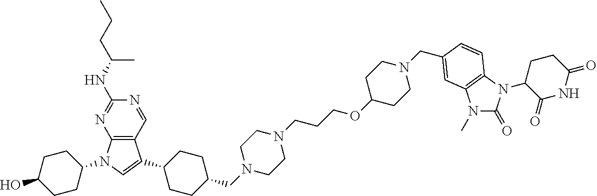 [SMILES: CCC[C@H](C)Nc1ncc2c(n1)n([C@H]1CC[C@H](O)CC1)cc2[C@H]1CC[C@@H](CN2CCN(CCCOC3CCN(Cc4ccc5c(c4)n(C)c(=O)n5C4CCC(=O)NC4=O)CC3)CC2)CC1] |
| I-208 | 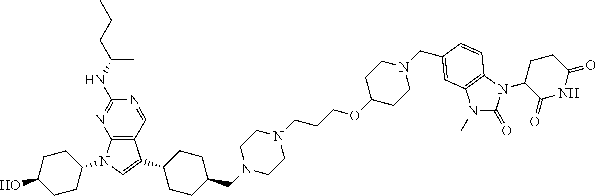 [SMILES: CCC[C@H](C)Nc1ncc2c(n1)n([C@H]1CC[C@H](O)CC1)cc2[C@H]1CC[C@H](CN2CCN(CCCOC3CCN(Cc4ccc5c(c4)n(C)c(=O)n5C4CCC(=O)NC4=O)CC3)CC2)CC1] |
| I-209 | 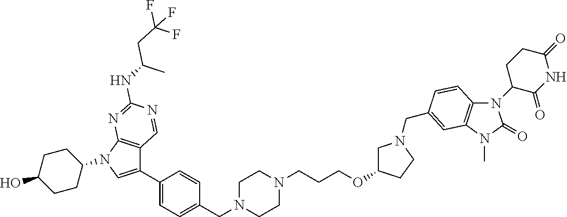 [SMILES: C[C@@H](CC(F)(F)F)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-210 | 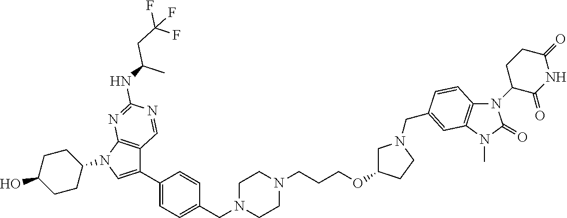 [SMILES: C[C@H](CC(F)(F)F)Nc1ncc2c(-c3ccc(CN4CCN(CCCO[C@H]5CCN(Cc6ccc7c(c6)n(C)c(=O)n7C6CCC(=O)NC6=O)C5)CC4)cc3)cn([C@H]3CC[C@H](O)CC3)c2n1] |
| I-211 | 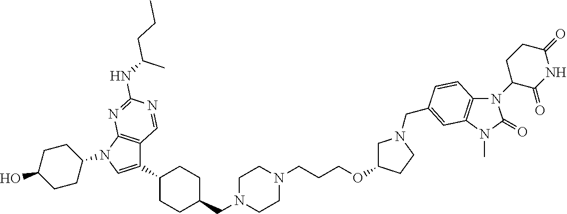 [SMILES: CCC[C@H](C)Nc1ncc2c(n1)n([C@H]1CC[C@H](O)CC1)cc2[C@H]1CC[C@H](CN2CCN(CCCO[C@H]3CCN(Cc4ccc5c(c4)n(C)c(=O)n5C4CCC(=O)NC4=O)C3)CC2)CC1] |
| I-212 | 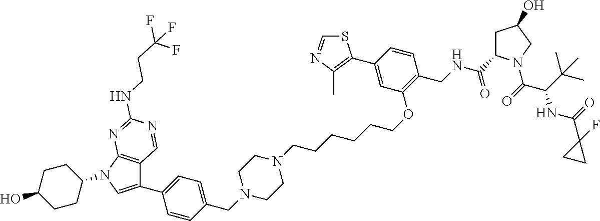 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCN2CCN(Cc3ccc(-c4cn([C@H]5CC[C@H](O)CC5)c5nc(NCCC(F)(F)F)ncc45)cc3)CC2)c1] |
| I-213 | 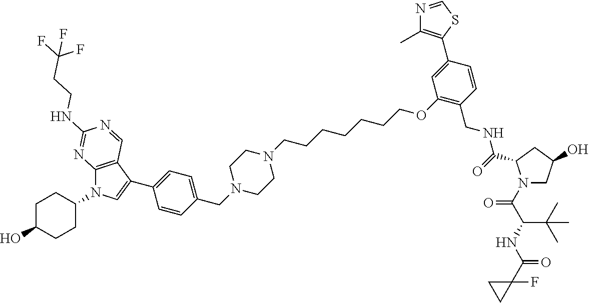 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCN2CCN(Cc3ccc(-c4cn([C@H]5CC[C@H](O)CC5)c5nc(NCCC(F)(F)F)ncc45)cc3)CC2)c1] |
| I-214 | 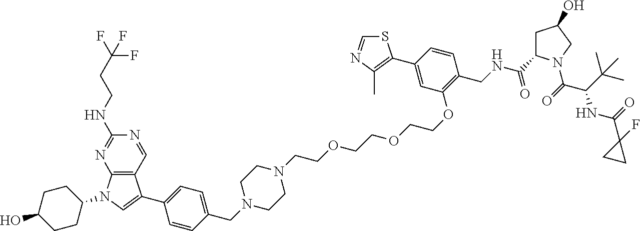 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCN2CCN(Cc3ccc(-c4cn([C@H]5CC[C@H](O)CC5)c5nc(NCCC(F)(F)F)ncc45)cc3)CC2)c1] |
| I-215 | 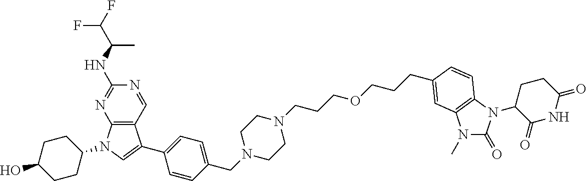 [SMILES: C[C@@H](Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1)C(F)F] |
| I-216 | 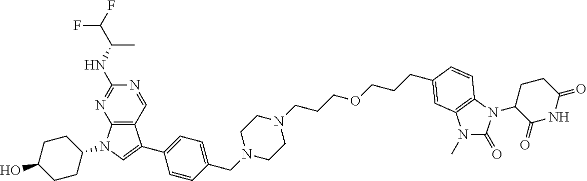 [SMILES: C[C@H](Nc1ncc2c(-c3ccc(CN4CCN(CCCOCCCc5ccc6c(c5)n(C)c(=O)n6C5CCC(=O)NC5=O)CC4)cc3)cn([C@H]3CC[C@H](O)CC3)c2n1)C(F)F] |
| I-217 | 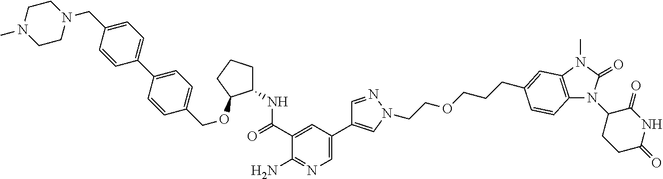 [SMILES: CN1CCN(Cc2ccc(-c3ccc(CO[C@H]4CCC[C@@H]4NC(=O)c4cc(-c5cnn(CCOCCCc6ccc7c(c6)n(C)c(=O)n7C6CCC(=O)NC6=O)c5)cnc4N)cc3)cc2)CC1] |
| I-218 | 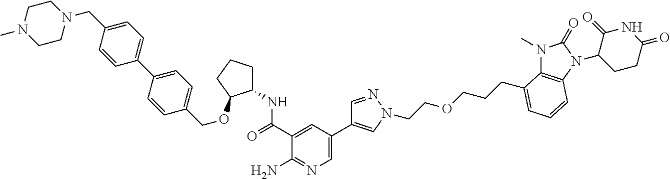 [SMILES: CN1CCN(Cc2ccc(-c3ccc(CO[C@H]4CCC[C@@H]4NC(=O)c4cc(-c5cnn(CCOCCCc6cccc7c6n(C)c(=O)n7C6CCC(=O)NC6=O)c5)cnc4N)cc3)cc2)CC1] |
| I-219 | 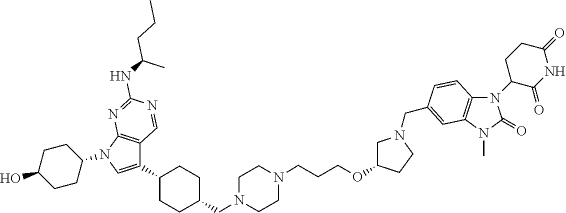 [SMILES: CCC[C@@H](C)Nc1ncc2c(n1)n([C@H]1CC[C@H](O)CC1)cc2[C@H]1CC[C@@H](CN2CCN(CCCO[C@H]3CCN(Cc4ccc5c(c4)n(C)c(=O)n5C4CCC(=O)NC4=O)C3)CC2)CC1] |
| I-220 | 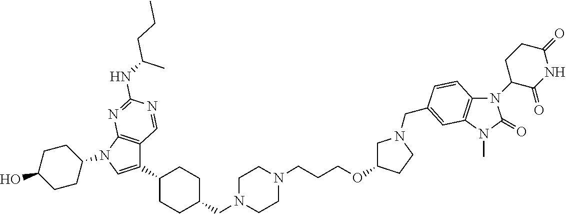 [SMILES: CCC[C@H](C)Nc1ncc2c(n1)n([C@H]1CC[C@H](O)CC1)cc2[C@H]1CC[C@@H](CN2CCN(CCCO[C@H]3CCN(Cc4ccc5c(c4)n(C)c(=O)n5C4CCC(=O)NC4=O)C3)CC2)CC1] |
| I-221 | 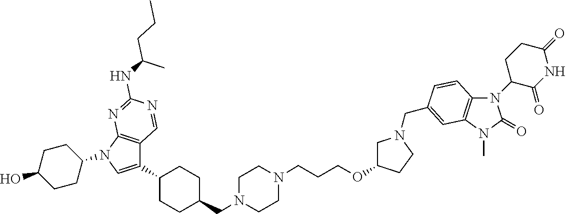 [SMILES: CCC[C@@H](C)Nc1ncc2c(n1)n([C@H]1CC[C@H](O)CC1)cc2[C@H]1CC[C@H](CN2CCN(CCCO[C@H]3CCN(Cc4ccc5c(c4)n(C)c(=O)n5C4CCC(=O)NC4=O)C3)CC2)CC1] |
| I-222 | 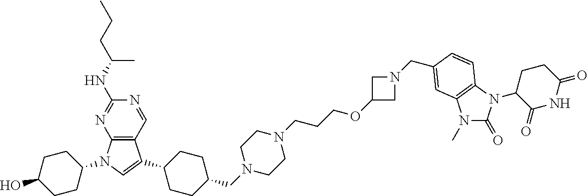 [SMILES: CCC[C@H](C)Nc1ncc2c(n1)n([C@H]1CC[C@H](O)CC1)cc2[C@H]1CC[C@@H](CN2CCN(CCCOC3CN(Cc4ccc5c(c4)n(C)c(=O)n5C4CCC(=O)NC4=O)C3)CC2)CC1] |
| I-223 | 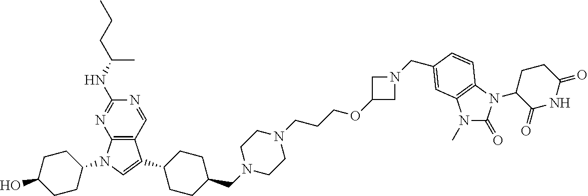 [SMILES: CCC[C@H](C)Nc1ncc2c(n1)n([C@H]1CC[C@H](O)CC1)cc2[C@H]1CC[C@H](CN2CCN(CCCOC3CN(Cc4ccc5c(c4)n(C)c(=O)n5C4CCC(=O)NC4=O)C3)CC2)CC1] |
| I-224 | 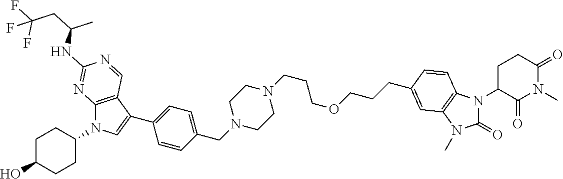 [SMILES: C[C@H](CC(F)(F)F)Nc1ncc2c(-c3ccc(CN4CCN(CCCOCCCc5ccc6c(c5)n(C)c(=O)n6C5CCC(=O)N(C)C5=O)CC4)cc3)cn([C@H]3CC[C@H](O)CC3)c2n1] |
| I-225 | 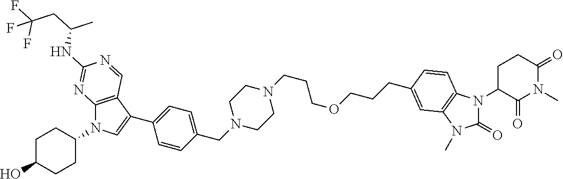 [SMILES: C[C@@H](CC(F)(F)F)Nc1ncc2c(-c3ccc(CN4CCN(CCCOCCCc5ccc6c(c5)n(C)c(=O)n6C5CCC(=O)N(C)C5=O)CC4)cc3)cn([C@H]3CC[C@H](O)CC3)c2n1] |
| I-226 | 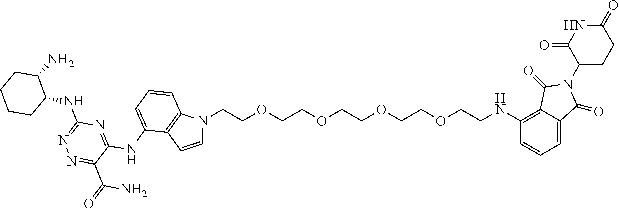 [SMILES: NC(=O)c1nnc(N[C@@H]2CCCC[C@@H]2N)nc1Nc1cccc2c1ccn2CCOCCOCCOCCOCCNc1cccc2c1C(=O)N(C1CCC(=O)NC1=O)C2=O] |
| I-227 | 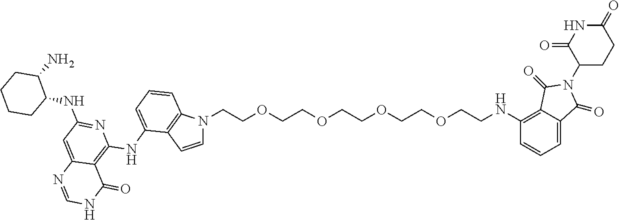 [SMILES: N[C@H]1CCCC[C@H]1Nc1cc2nc[nH]c(=O)c2c(Nc2cccc3c2ccn3CCOCCOCCOCCOCCNc2cccc3c2C(=O)N(C2CCC(=O)NC2=O)C3=O)n1] |
| I-228 | 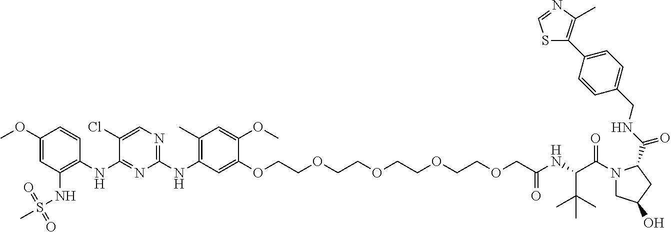 [SMILES: COc1ccc(Nc2nc(Nc3cc(OCCOCCOCCOCCOCC(=O)N[C@H](C(=O)N4C[C@H](O)C[C@H]4C(=O)NCc4ccc(-c5scnc5C)cc4)C(C)(C)C)c(OC)cc3C)ncc2Cl)c(NS(C)(=O)=O)c1] |
| I-229 | 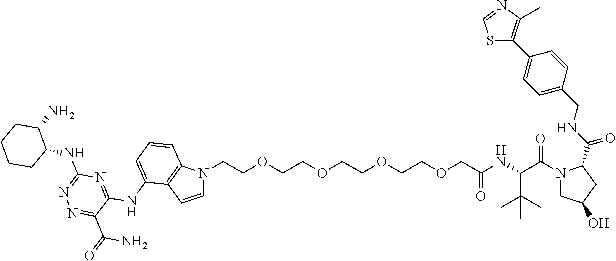 [SMILES: Cc1ncsc1-c1ccc(CNC(=O)[C@@H]2C[C@@H](O)CN2C(=O)[C@@H](NC(=O)COCCOCCOCCOCCn2ccc3c(Nc4nc(N[C@@H]5CCCC[C@@H]5N)nnc4C(N)=O)cccc32)C(C)(C)C)cc1] |
| I-230 | 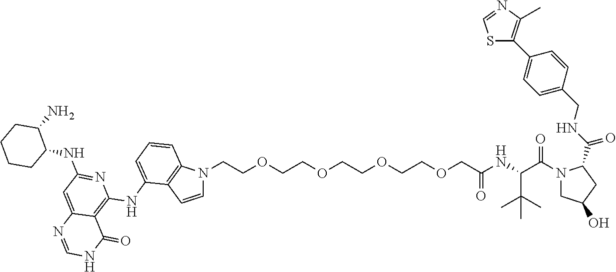 [SMILES: Cc1ncsc1-c1ccc(CNC(=O)[C@@H]2C[C@@H](O)CN2C(=O)[C@@H](NC(=O)COCCOCCOCCOCCn2ccc3c(Nc4nc(N[C@@H]5CCCC[C@@H]5N)cc5nc[nH]c(=O)c45)cccc32)C(C)(C)C)cc1] |
| I-231 | 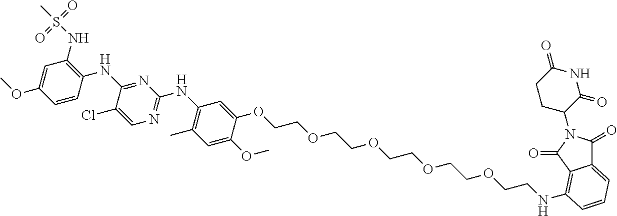 [SMILES: COc1ccc(Nc2nc(Nc3cc(OCCOCCOCCOCCOCCNc4cccc5c4C(=O)N(C4CCC(=O)NC4=O)C5=O)c(OC)cc3C)ncc2Cl)c(NS(C)(=O)=O)c1] |
| I-232 | 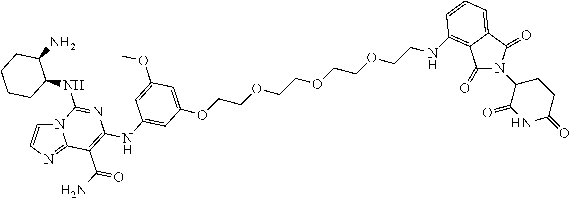 [SMILES: COc1cc(Nc2nc(N[C@H]3CCCC[C@H]3N)n3ccnc3c2C(N)=O)cc(OCCOCCOCCOCCNc2cccc3c2C(=O)N(C2CCC(=O)NC2=O)C3=O)c1] |
| I-233 | 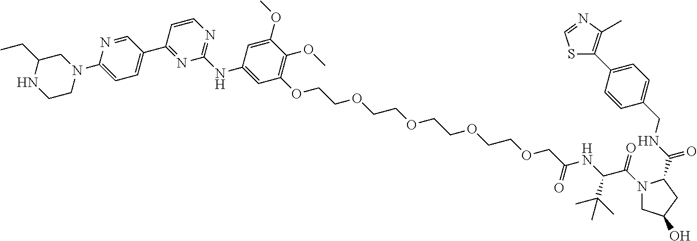 [SMILES: CCC1CN(c2ccc(-c3ccnc(Nc4cc(OC)c(OC)c(OCCOCCOCCOCCOCC(=O)N[C@H](C(=O)N5C[C@H](O)C[C@H]5C(=O)NCc5ccc(-c6scnc6C)cc5)C(C)(C)C)c4)n3)cn2)CCN1] |
| I-234 | 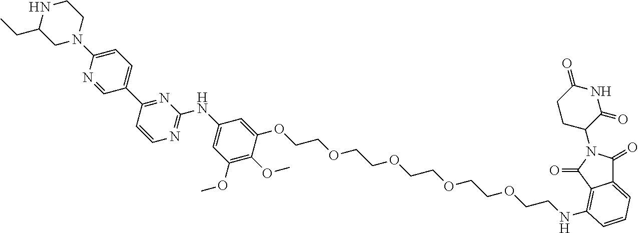 [SMILES: CCC1CN(c2ccc(-c3ccnc(Nc4cc(OC)c(OC)c(OCCOCCOCCOCCOCCNc5cccc6c5C(=O)N(C5CCC(=O)NC5=O)C6=O)c4)n3)cn2)CCN1] |
| I-235 | 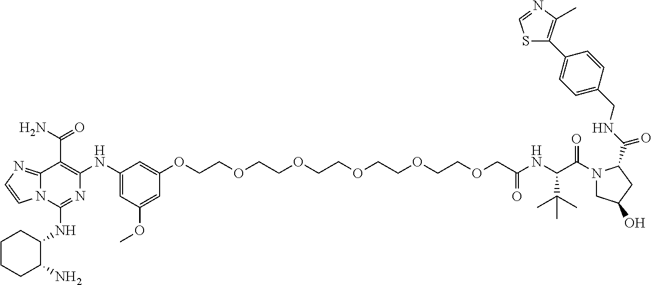 [SMILES: COc1cc(Nc2nc(N[C@H]3CCCC[C@H]3N)n3ccnc3c2C(N)=O)cc(OCCOCCOCCOCCOCCOCC(=O)N[C@H](C(=O)N2C[C@H](O)C[C@H]2C(=O)NCc2ccc(-c3scnc3C)cc2)C(C)(C)C)c1] |
| I-236 | 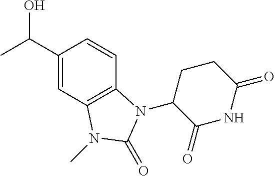 [SMILES: CC(O)c1ccc2c(c1)n(C)c(=O)n2C1CCC(=O)NC1=O] |

In some embodiments, the present invention provides a compound set forth in Table 1, above, or a pharmaceutically acceptable salt thereof.

### 4. General Methods of Providing the Present Compounds

The compounds of this invention may be prepared or isolated in general by synthetic and/or semi-synthetic methods known to those skilled in the art for analogous compounds and by methods described in detail in the Examples, herein.

In the Schemes below, where a particular protecting group, leaving group, or transformation condition is depicted, one of ordinary skill in the art will appreciate that other protecting groups, leaving groups, and transformation conditions are also suitable and are contemplated. Such groups and transformations are described in detail in March's Advanced Organic Chemistry: Reactions, Mechanisms, and Structure, M. B. Smith and J. March, 5^{th} Edition, John Wiley & Sons, 2001, Comprehensive Organic Transformations, R. C. Larock, 2^{nd} Edition, John Wiley & Sons, 1999, and Protecting Groups in Organic Synthesis, T. W. Greene and P. G. M. Wuts, 3^{rd} edition, John Wiley & Sons, 1999, the entirety of each of which is hereby incorporated herein by reference.

As used herein, the phrase “oxygen protecting group” includes, for example, carbonyl protecting groups, hydroxyl protecting groups, etc. Hydroxyl protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Examples of suitable hydroxyl protecting groups include, but are not limited to, esters, allyl ethers, ethers, silyl ethers, alkyl ethers, arylalkyl ethers, and alkoxyalkyl ethers. Examples of such esters include formates, acetates, carbonates, and sulfonates. Specific examples include formate, benzoyl formate, chloroacetate, trifluoroacetate, methoxyacetate, triphenylmethoxyacetate, p-chlorophenoxyacetate, 3-phenylpropionate, 4-oxopentanoate, 4,4-(ethylenedithio)pentanoate, pivaloate (trimethylacetyl), crotonate, 4-methoxy-crotonate, benzoate, p-benylbenzoate, 2,4,6-trimethylbenzoate, carbonates such as methyl, 9-fluorenylmethyl, ethyl, 2,2,2-trichloroethyl, 2-(trimethylsilyl)ethyl, 2-(phenylsulfonyl)ethyl, vinyl, allyl, and p-nitrobenzyl. Examples of such silyl ethers include trimethylsilyl, triethylsilyl, t-butyldimethylsilyl, t-butyldiphenylsilyl, triisopropylsilyl, and other trialkylsilyl ethers. Alkyl ethers include methyl, benzyl, p-methoxybenzyl, 3,4-dimethoxybenzyl, trityl, t-butyl, allyl, and allyloxycarbonyl ethers or derivatives. Alkoxyalkyl ethers include acetals such as methoxymethyl, methylthiomethyl, (2-methoxyethoxy)methyl, benzyloxymethyl, beta-(trimethylsilyl)ethoxymethyl, and tetrahydropyranyl ethers. Examples of arylalkyl ethers include benzyl, p-methoxybenzyl (MPM), 3,4-dimethoxybenzyl, O-nitrobenzyl, p-nitrobenzyl, p-halobenzyl, 2,6-dichlorobenzyl, p-cyanobenzyl, and 2- and 4-picolyl.

Amino protecting groups are well known in the art and include those described in detail in Protecting Groups in Organic Synthesis, T. W. Greene and P. G. M. Wuts, 3^{rd} edition, John Wiley & Sons, 1999, the entirety of which is incorporated herein by reference. Suitable amino protecting groups include, but are not limited to, aralkylamines, carbamates, cyclic imides, allyl amines, amides, and the like. Examples of such groups include t-butyloxycarbonyl (BOC), ethyloxycarbonyl, methyl oxycarbonyl, trichloroethyloxycarbonyl, allyloxycarbonyl (Alloc), benzyloxocarbonyl (CBZ), allyl, phthalimide, benzyl (Bn), fluorenylmethylcarbonyl (Fmoc), formyl, acetyl, chloroacetyl, dichloroacetyl, trichloroacetyl, phenylacetyl, trifluoroacetyl, benzoyl, and the like.

In certain embodiments, compounds of the present invention are generally prepared according to Scheme 1 set forth below:

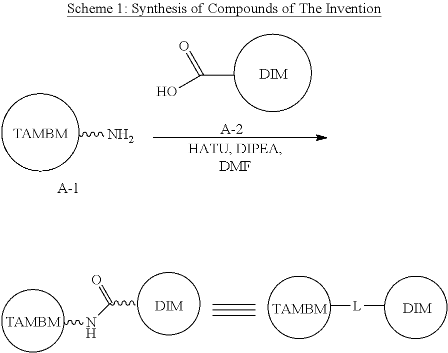

As depicted in Scheme 1, above, amine A-1 is coupled to acid A-2 using the coupling agent HATU in the presence of the base DIPEA in DMF to form a provided compound with a linker comprising an amide bond. The squiggly bond, [symbol] , represents the portion of the linker between TAMBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-C1, DEPBT, T3P, TATU, TBTU, TNTU, TOTU, TPTU, TSTU, or TDBTU.

In certain embodiments, compounds of the present invention are generally prepared according to Scheme 2 set forth below:

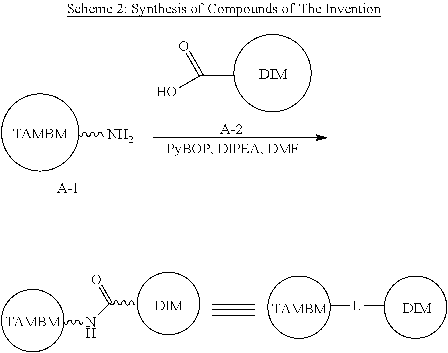

As depicted in Scheme 2, above, amine A-1 is coupled to acid A-2 using the coupling agent PyBOP in the presence of the base DIPEA in DMF to form a provided compound with a linker comprising an amide bond. The squiggly bond, [symbol] , represents the portion of the linker between TAMBM and the terminal amino group of A-1 or the portion of the linker between DIM and the terminal carboxyl group of A-2, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 3 set forth below:

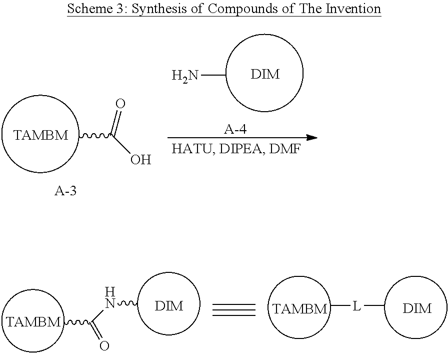

As depicted in Scheme 3, above, acid A-3 is coupled to amine A-4 using the coupling agent HATU in the presence of the base DIPEA in DMF to form a provided compound with a linker comprising an amide bond. The squiggly bond, [symbol] , represents the portion of the linker between TAMBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 4 set forth below:

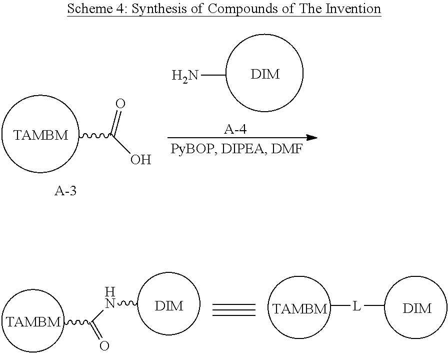

As depicted in Scheme 4, above, acid A-3 is coupled to amine A-4 using the coupling agent PyBOP in the presence of the base DIPEA in DMF to form a provided compound with a linker comprising an amide bond. The squiggly bond, [symbol] , represents the portion of the linker between TAMBM and the terminal carboxyl group of A-3 or the portion of the linker between DIM and the terminal amino group of A-4, respectively. Additionally, an amide bond can be formed using coupling reagents known in the art such as, but not limited to DCC, DIC, EDC, HBTU, HCTU, PyAOP, PyBrOP, BOP, BOP-Cl, DEPBT, T3P, TATU, TBTU, TNTU, TOTU, TPTU, TSTU, or TDBTU.

In certain embodiments, compounds of the present invention are generally prepared according to Scheme 5 set forth below:

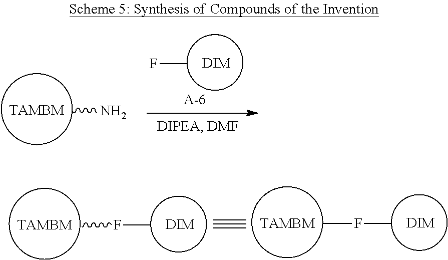

As depicted in Scheme 5, above, an SNAr displacement of fluoride A-6 by amine A-5 is effected in the presence of the base DIPEA in DMF to form a provided compound with a linker comprising a secondary amine. The squiggly bond, [symbol] , represents the portion of the linker between TAMBM and the terminal amino group of A-5.

In certain embodiments, compounds of the present invention are generally prepared according to Scheme 6 set forth below:

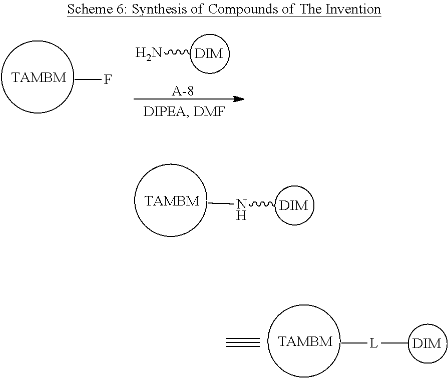

As depicted in Scheme 6, above, an SNAr displacement of fluoride A-7 by amine A-8 is effected in the presence of the base DIPEA in DMF to form a provided compound with a linker comprising a secondary amine. The squiggly bond, [symbol] , represents the portion of the linker between DIM and the terminal amino group of A-8.

In certain embodiments, compounds of the present invention are generally prepared according to Scheme 7 set forth below:

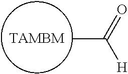

As depicted in Scheme 7, above, reductive alkylation of aldehyde A-9 by amine A-10 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between DIM and the terminal amino group of A-10.

In certain embodiments, compounds of the present invention are generally prepared according to Scheme 8 set forth below:

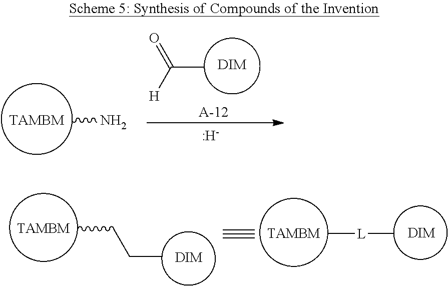

As depicted in Scheme 8, above, reductive alkylation of aldehyde A-12 by amine A-11 is effected in the presence of a mild hydride source (e.g., sodium cyanoborohydride or sodium triacetoxyborohydride) to form a provided compound with a linker comprising a secondary amine. The squiggly bond, [symbol] , represents the portion of the linker between TAMBM and the terminal amino group of A-11.

One of skill in the art will appreciate that various functional groups present in compounds of the invention such as aliphatic groups, alcohols, carboxylic acids, esters, amides, aldehydes, halogens and nitriles can be interconverted by techniques well known in the art including, but not limited to reduction, oxidation, esterification, hydrolysis, partial oxidation, partial reduction, halogenation, dehydration, partial hydration, and hydration. “March's Advanced Organic Chemistry”, 5^{th} Ed., Ed.: Smith, M. B. and March, J., John Wiley & Sons, New York: 2001, the entirety of which is incorporated herein by reference. Such interconversions may require one or more of the aforementioned techniques, and certain methods for synthesizing compounds of the invention are described below in the Exemplification.

### 5. Uses, Formulation and Administration

Pharmaceutically Acceptable Compositions

According to another embodiment, the invention provides a composition comprising a compound of this invention or a pharmaceutically acceptable derivative thereof and a pharmaceutically acceptable carrier, adjuvant, or vehicle. The amount of compound in compositions of this invention is such that is effective to measurably degrade and/or inhibit a TAM receptor kinase, or a mutant thereof, in a biological sample or in a patient. In some embodiments, the TAM receptor kinase is Tyro3. In some embodiments, the TAM receptor kinase is Axl. In some embodiments, the TAM receptor kinase is MerTK. In certain embodiments, the amount of compound in compositions of this invention is such that is effective to measurably degrade and/or inhibit a TAM receptor kinase, or a mutant thereof, in a biological sample or in a patient. In certain embodiments, a composition of this invention is formulated for administration to a patient in need of such composition. In some embodiments, a composition of this invention is formulated for oral administration to a patient.

The term “patient,” as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this invention that, upon administration to a recipient, is capable of providing, either directly or indirectly, a compound of this invention or an inhibitorily or degratorily active metabolite or residue thereof.

As used herein, the term “inhibitorily active metabolite or residue thereof” means that a metabolite or residue thereof is also an inhibitor of a TAM receptor kinase, or a mutant thereof.

As used herein, the term “degratorily active metabolite or residue thereof” means that a metabolite or residue thereof is also a degrader of a TAM receptor kinase, or a mutant thereof

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Most preferably, pharmaceutically acceptable compositions of this invention are formulated for oral administration. Such formulations may be administered with or without food. In some embodiments, pharmaceutically acceptable compositions of this invention are administered without food. In other embodiments, pharmaceutically acceptable compositions of this invention are administered with food.

The amount of compounds of the present invention that may be combined with the carrier materials to produce a composition in a single dosage form will vary depending upon the host treated, the particular mode of administration. Preferably, provided compositions should be formulated so that a dosage of between 0.01-100 mg/kg body weight/day of the compound can be administered to a patient receiving these compositions.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

Uses of Compounds and Pharmaceutically Acceptable Compositions

Compounds and compositions described herein are generally useful for the degradation and/or inhibition of kinase activity of one or more enzymes.

Examples of kinases that are degraded and/or inhibited by the compounds and compositions described herein and against which the methods described herein are useful include receptor tyrosine kinases, such as, a TAM receptor kinase (e.g., MerTK; also known as Mer).

The activity of a compound utilized in this invention as a degrader and/or inhibitor of a TAM receptor kinase or a mutant thereof, may be assayed in vitro, in vivo or in a cell line. In vitro assays include assays that determine inhibition of either the phosphorylation activity and/or the subsequent functional consequences, or ATPase activity of an activated TAM receptor kinase (e.g., MerTK), or a mutant thereof Alternate in vitro assays quantitate the ability of the inhibitor to bind to a TAM receptor kinase. Inhibitor binding may be measured by radiolabeling the inhibitor prior to binding, isolating the inhibitor/TAM receptor complex and determining the amount of radiolabel bound. Alternatively, inhibitor binding may be determined by running a competition experiment where new inhibitors are incubated with a TAM receptor kinase bound to known radioligands.

Representative in vitro and in vivo assays useful in assaying a TAM receptor kinase inhibitor include those described and disclosed in, e.g., Yan, S. B. et al., LY2801653 is an orally bioavailable multi-kinase inhibitor with potent activity against MET, MST 1R, and other oncoproteins, and displays anti-tumor activities in mouse xenograft models, Invest. New Drugs (2013) 31(4): 833-44; Knubel, K. H. et al., MerTK inhibition is a novel therapeutic approach for glioblastoma multiforme, Oncotarget (2014) 5(5): 1338-51; Page, L. S. et al., MerTK Receptor Tyrosine Kinase Inhibition As a Potential Strategy to Augment Immune-Mediated Clearance of Acute Myeloid Leukemia, Blood (2016) 128(22): 4044-48; Minson, K. A. et al., The MERTK/FLT3 inhibitor MRX-2843 overcomes resistance-conferring FLT3 mutations in acute myeloid leukemia, JCI Insight. (2016) 1(3): e85630, https://doi.org/10.1172/jci.insight.85630; Kim, J. E. et al., MerTK inhibition by RXDX-106 in MerTK activated gastric cancer cell lines, Oncotarget (2017) 8(62): 105727-34; Zhang, W. et al., UNC2025, a Potent and Orally Bioavailable MER/FLT3 Dual Inhibitor, J. Med. Chem. (2014) 57(16): 7031-41; Huey, M. G. et al., Targeting the TAM Receptors in Leukemia, Cancers (2016) 8(11): 101/1-101/22; DeRyckere, A. B. et al., UNC2025, a MERTK Small-Molecule Inhibitor, Is Therapeutically Effective Alone and in Combination with Methotrexate in Leukemia Models, Clin. Cancer Res. (2017) 23(6): 1481-92; Sufit, A. et al., MERTK Inhibition Induces Polyploidy and Promotes Cell Death and Cellular Senescence in Glioblastoma Multiforme, PLoS One (2016) 11(10); e0165107/1-20; Cummings, C. T. et al., Small Molecule Inhibition of MERTK is Efficacious in Non-Small Cell lung Cancer Models Independent of Driver Oncogene Status, Mol. Cancer. Ther. (2015) 14(9): 2014-22; Shi, C. et al., The proto-oncogene Mer tyrosine kinase is a novel therapeutic target in mantle cell lymphoma, J. Hematol. Oncol. (2018) 11(1): 43; Branchford, B. R. et al., The small-molecule MERTK inhibitor UNC2025 decreases platelet activation and prevents thrombosis, J. Thromb. Haemost. (2018) 16(2): 352-63; Paolino, M. et al., The E3 ligase Cbl-b and TAM receptors regulate cancer metastasis via natural killer cells, Nature (2014) 507(7493): 508-12; Cummings, C. T. et al., Molecular Pathways: MERTK Signaling in Cancer, Clin. Cancer Res. (2013) 19(19): 5275-80; Schlegel, J. et al., MERTK receptor tyrosine kinase is a therapeutic target in melanoma, J. Clin. Invest. (2013) 123(5): 2257-67; Ho, Y. J. et al., MerTK is a novel therapeutic target in gastric cancer, Oncotarget (2017) 8(57): 96656-67; Espindola, M. S. et al., Targeting TAM Receptors Ameliorates Fibrotic Mechanisms in Idiopathic Pulmonary Fibrosis, Am. J. Resp. Critical Med. (2018) 197(11): 1443-56; Mikaella, V. & Sassan, H., TAM Receptor Tyrosine Kinases in Cancer Drug Resistance, Cancer Res. (2017) 77(11): 2775-8; Kimani, S. G. et al., Small molecule inhibitors block Gas6-inducible TAM activation and tumorigenicity, Scientific Reports (2017) 7: 74908; Schoumacher, M. & Burbridge, M., Key Roles of AXL and MER Receptor Tyrosine Kinases in Resistance to Multiple Anticancer Therapies, Curr. Oncol. Rep. (2017) 19(3):19; Davra, V. et al., Ligand Activation of TAM Family Receptors-Implications for Tumor Biology and Therapeutic Response, Cancers (Basel) (2016) 8(12): 107; von Mässenhaausen, A. et al., MERTK as a novel therapeutic target in head and neck cancer, Oncotarget (2016) 7(22): 32678-94; Lee-Sherick, A. B. et al., Efficacy of a Mer and Flt3 tyrosine kinase small molecule inhibitor, UNC1666, in acute myeloid leukemia, Oncotarget (2015) 6(9): 6722-36; Christoph, S. et al., UNC569, a novel small-molecule mer inhibitor with efficacy against acute lymphoblastic leukemia in vitro and in vivo, Mol. Cancer. Ther. (2013) 12(11): 2367-77; Liu, J. et al., UNC1062, a new and potent Mer inhibitor, Eur. J. Med. Chem. (2013) 65: 83-93;

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

Provided compounds are degraders and/or inhibitors of a TAM receptor kinase and are therefore useful for treating one or more disorders associated with activity of a TAM receptor kinase (e.g., MerTK). Thus, in certain embodiments, the present invention provides a method for treating a TAM receptor kinase-mediated disorder comprising the step of administering to a patient in need thereof a compound of the present invention, or pharmaceutically acceptable composition thereof. In some embodiments, the TAM receptor kinase is Tyro3. In some embodiments, the TAM receptor kinase is Axl. In some embodiments, the TAM receptor kinase is MerTK.

As used herein, the term “TAM receptor kinase-mediated” disorders, diseases, and/or conditions as used herein means any disease or other deleterious condition in which Tyro3, Axl, and/or MerTK, or mutants thereof, are known to play a role. Accordingly, another embodiment of the present invention relates to treating or lessening the severity of one or more diseases in which Tyro3, Axl, and/or MerTK, or mutants thereof, are known to play a role.

As used herein, the term “MerTK-mediated” disorders, diseases, and/or conditions as used herein means any disease or other deleterious condition in which MerTK, or a mutant thereof, is known to play a role. Accordingly, another embodiment of the present invention relates to treating or lessening the severity of one or more diseases in which MerTK, or a mutant thereof, is known to play a role.

In some embodiments, the present invention provides a method for treating one or more disorders, diseases, and/or conditions wherein the disorder, disease, or condition is an infectious disease, an immune disorder, an autoimmune disorder, an inflammatory disorder, a proliferation disorder, or a platelet aggregation disorder.

Compounds of the present invention are useful in the treatment of an infectious disease. In some embodiments, the infectious disease is a viral infection. In some embodiments, the infectious disease is a bacterial infection.

In some embodiments, the viral infection is caused by a viral agent selected from, for example, Flaviviridae viruses (e.g., Yellow Fever, West Nile, and Dengue); Hepacivirus (Hepatitis C); Pegivirus and Pestivirus (bovine viral diarrhea virus); Filoviridae viruses (e.g., Chikungunya virus); Coronaviruses (e.g., severe acute respiratory syndrome (SARS) and Middle East respiratory syndrome (MERS)); Orthomyxoviridae viruses (e.g., Respiratory syncytial virus (RSB), measles, and mumps); and Calciviridae viruses (e.g., Lagovirus, Vesivirus, Sapovirus, Norovirus, and Lentiviruses (e.g., HIV)).

In some embodiments, the viral infection is caused by an enveloped virus, including: Bornaviridae; Bunyaviridae (e.g., La Crosse virus and Hantaan); Coronaviridae (e.g., oronaviruses, or Toroviruses); Filoviridae (e.g., Ebola and Marburg); Flaviviridae (e.g., Dengue, encephalitis viruses including (e.g., West Nile virus and Japanese encephalitis virus) and Yellow Fever); Hepadnaviridae; Herpesviridae (e.g., cytomegalovirus, herpes simplex viruses 1 and 2, HHV-6 HHV-7 HHV-8 pseudorabies virus, and varicella zoster virus); Nyamiviridae; Orthomyxoviridae (e.g., influenza virus); Paramyxoviridae (e.g., measles, metapneumovirus, mumps, parainfluenza, respiratory syncytial virus, and sendai); Poxviridae (e.g., pox viruses (e.g., smallpox, monkey pox), Molluscum contagiosum virus, variola viruses, vaccinia virus, and yatapox viruses (e.g., Tanapox and Yabapox)); Retroviridae (e.g., Coltiviruses (e.g., CTFV and Banna virus), human immunodeficiency viruses (e.g., HIV-1 and HIV-2), murine leukemia virus, simian immunodeficiency virus, feline immunodeficiency virus, human T-cell leukemia viruses 1 and 2, and XMRV); Rhabdoviridae (e.g., vesicular stomatitis and rabies); and Togaviridae (e.g., rubella viruses or alpha viruses (e.g., Chikungunya virus, Eastern equine encephalitis virus, O'nyong'nyong virus, Ross River virus, Semliki Forest virus, Sindbis, Venezuelan equine encephalitis or Western equine encephalitis virus)).

In some embodiments, the bacterial infection is caused by Gram-negative bacteria, including: Escherichia coli, Salmonella, and other Enterobacteriaceae, Pseudomonas, Moraxella, Helicobacter, Stenotrophomonas, Bdellovibrio, acetic acid bacteria, Legionella, Staphylococcus aureus, Hemophilus influenzae, Klebsiella pneumoniae, Legionella pneumophila, Pseudomonas aeruginosa, Vibrio cholerae, Proteus mirabilis, Enterobacter cloacae, Serratia marcescens, Clostridium tetani, Helicobacter pylori, Salmonella enteritidis, Salmonella typhi, Shigella flexneri, or Acinetobacter baumanii.

In some embodiment, the compounds and methods of the present invention are useful for the treatment of immune and inflammatory disorders, diseases, and conditions. In some embodiments, the compounds of the present invention reverse the MerTK-induced suppression of proinflammatory cytokines such as, for example, wound healing cytokines (IL-10 and GAS6) and enhance the expression of acute inflammatory cytokines (IL-12 and IL-6). In some embodiments, the compounds of the present invention modulate immune response to cancer.

In some embodiments, the bacterial infection is caused by Gram-positive bacteria, including: Bacillus, Listeria, Staphylococcus, Enterococcus, Lactobacillus, Lactococcus, Leuconostoc, Pedicoccus, Streptococcus, Acetobacterium, Clostridium, Eubacterium, Heliobacterium, Heliospirillum, Megasphaera, Pectinatus, Selenomonas, Zymophilus, Sporomusa, Mycoplasma, Spiroplasma, Ureaplasma, or Erysipelothrix.

In certain embodiments, the compounds and methods of the present invention are useful for the treatment of a proliferative disorder. In certain embodiments, the compounds and methods of the present invention are useful for the treatment of cancer. In some embodiments, the cancer treated is a primary tumor or a metastatic tumor. In some embodiments, present invention is used to treat: solid tumor (e.g., melanoma); lung cancer (e.g., lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma); breast cancer (e.g., ductal carcinoma, lobular carcinoma, inflammatory breast cancer, clear cell carcinoma, mucinous carcinoma, serosal cavities breast carcinoma); colorectal cancer (e.g., colon cancer, rectal cancer, colorectal adenocarcinoma); anal cancer; pancreatic cancer (e.g., pancreatic adenocarcinoma, islet cell carcinoma, neuroendocrine tumors); prostate cancer; prostate adenocarcinoma; ovarian carcinoma (e.g., ovarian epithelial carcinoma or surface epithelial-stromal tumor (e.g., serous tumor, endometrioid tumor and mucinous cystadenocarcinoma, sex-cord-stromal tumor)); liver and bile duct carcinoma (e.g., hepatocellular carcinoma, cholangiocarcinoma, hemangioma); esophageal carcinoma (e.g., esophageal adenocarcinoma and squamous cell carcinoma); oral and oropharyngeal squamous cell carcinoma; salivary gland adenoid cystic carcinoma; bladder cancer; bladder carcinoma; carcinoma of the uterus (e.g., endometrial adenocarcinoma, ocular, uterine papillary serous carcinoma, uterine clear-cell carcinoma, uterine sarcomas, and leiomyosarcomas, mixed mullerian tumors); glioma, glioblastoma, medulloblastoma, and other tumors of the brain; kidney cancers (e.g., renal cell carcinoma, clear cell carcinoma, Wilm's tumor); cancer of the head and neck (e.g., squamous cell carcinomas); cancer of the stomach (e.g., gastric cancers, stomach adenocarcinoma, gastrointestinal stromal tumor); testicular cancer; germ cell tumor; neuroendocrine tumor; cervical cancer; carcinoids of the gastrointestinal tract, breast, and other organs; signet ring cell carcinoma; mesenchymal tumors (e.g., sarcomas), fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin (e.g., melanoma); cervical; retinoblastoma; head and neck cancer; pancreatic; brain; thyroid; testicular; renal; bladder; soft tissue; adrenal gland; urethra; cancers of the penis; myxosarcoma; chondrosarcoma; osteosarcoma; chordoma; malignant fibrous histiocytoma; lymphangiosarcoma; mesothelioma; squamous cell carcinoma; epidermoid carcinoma; malignant skin adnexal tumors; adenocarcinoma; hepatoma; hepatocellular carcinoma; renal cell carcinoma; hypernephroma; cholangiocarcinoma; transitional cell carcinoma; choriocarcinoma; seminoma; embryonal cell carcinoma; glioma anaplastic; glioblastoma multiforme; neuroblastoma; medulloblastoma; malignant meningioma; malignant schwannoma; neurofibrosarcoma; parathyroid carcinoma; medullary carcinoma of thyroid; bronchial carcinoid; pheochromocytoma; Islet cell carcinoma; malignant carcinoid; malignant paraganglioma; melanoma; Merkel cell neoplasm; cystosarcoma phylloide, salivary cancers, thymic carcinomas; and cancers of the vagina among others.

In some embodiments, the cancer treated is a lymphoma, or lymphocytic or myelocytic proliferation disorder or abnormality. In some embodiments the lymphoma is selected from: AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

In some embodiments, the compounds and methods of the present invention are useful in the treatment of blood clot formation. In some embodiments, the blood clot is a result of or a risk of a patient suffering from coronary artery disease, peripheral vascular disease, cerebrovascular disease, stable and unstable angina pectoris (SAP and UAP, respectively), left ventricular dysfunction LVD, (congestive) heart failure (CHF), myocardial death, myocardial infarction, atrial fibrillation, stroke, renal damage, percutaneous translumenal coronary angioplasty, athreosclerosis, disseminated intravascular coagulation, sepsis, endotoxemia (i.e., the presence of endotoxins in the blood), pulmonary embolism and deep vein thrombosis

Furthermore, the invention provides the use of a compound according to the definitions herein, or a pharmaceutically acceptable salt, or a hydrate or solvate thereof for the preparation of a medicament for the treatment of a proliferative disease, an inflammatory disease, an obstructive respiratory disease, a cardiovascular disease, a metabolic disease, a neurological disease, a neurodegenerative disease, a viral disease, or a disorder commonly occurring in connection with transplantation.

Combination Therapies

Depending upon the particular condition, or disease, to be treated, additional therapeutic agents, which are normally administered to treat that condition, may be administered in combination with compounds and compositions of this invention. As used herein, additional therapeutic agents that are normally administered to treat a particular disease, or condition, are known as “appropriate for the disease, or condition, being treated.”

In certain embodiments, a provided combination, or composition thereof, is administered in combination with another therapeutic agent.

In some embodiments, the present invention provides a method of treating a disclosed disease or condition comprising administering to a patient in need thereof an effective amount of a compound disclosed herein or a pharmaceutically acceptable salt thereof and co-administering simultaneously or sequentially an effective amount of one or more additional therapeutic agents, such as those described herein. In some embodiments, the method includes co-administering one additional therapeutic agent. In some embodiments, the method includes co-administering two additional therapeutic agents. In some embodiments, the combination of the disclosed compound and the additional therapeutic agent or agents acts synergistically.

Examples of agents the combinations of this invention may also be combined with include, without limitation: treatments for Alzheimer's Disease such as Aricept® and Excelon®; treatments for HIV such as ritonavir; treatments for Parkinson's Disease such as L-DOPA/carbidopa, entacapone, ropinrole, pramipexole, bromocriptine, pergolide, trihexephendyl, and amantadine; agents for treating Multiple Sclerosis (MS) such as beta interferon (e.g., Avonex® and Rebif®), Copaxone®, and mitoxantrone; treatments for asthma such as albuterol and Singulair®; agents for treating schizophrenia such as zyprexa, risperdal, seroquel, and haloperidol; anti-inflammatory agents such as corticosteroids, TNF blockers, IL-1 RA, azathioprine, cyclophosphamide, and sulfasalazine; immunomodulatory and immunosuppressive agents such as cyclosporin, tacrolimus, rapamycin, mycophenolate mofetil, interferons, corticosteroids, cyclophophamide, azathioprine, and sulfasalazine; neurotrophic factors such as acetylcholinesterase inhibitors, MAO inhibitors, interferons, anti-convulsants, ion channel blockers, riluzole, and anti-Parkinsonian agents; agents for treating cardiovascular disease such as beta-blockers, ACE inhibitors, diuretics, nitrates, calcium channel blockers, and statins; agents for treating liver disease such as corticosteroids, cholestyramine, interferons, and anti-viral agents; agents for treating blood disorders such as corticosteroids, anti-leukemic agents, and growth factors; agents that prolong or improve pharmacokinetics such as cytochrome P450 inhibitors (i.e., inhibitors of metabolic breakdown) and CYP3A4 inhibitors (e.g., ketokenozole and ritonavir), and agents for treating immunodeficiency disorders such as gamma globulin.

In certain embodiments, combination therapies of the present invention, or a pharmaceutically acceptable composition thereof, are administered in combination with a monoclonal antibody or an siRNA therapeutic.

Those additional agents may be administered separately from a provided combination therapy, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a combination of the present invention may be administered with another therapeutic agent simultaneously or sequentially in separate unit dosage forms or together in a single unit dosage form.

The amount of additional therapeutic agent present in the compositions of this invention will be no more than the amount that would normally be administered in a composition comprising that therapeutic agent as the only active agent. Preferably the amount of additional therapeutic agent in the presently disclosed compositions will range from about 50% to 100% of the amount normally present in a composition comprising that agent as the only therapeutically active agent.

One or more other therapeutic agent may be administered separately from a compound or composition of the invention, as part of a multiple dosage regimen. Alternatively, one or more other therapeutic agents agents may be part of a single dosage form, mixed together with a compound of this invention in a single composition. If administered as a multiple dosage regime, one or more other therapeutic agent and a compound or composition of the invention may be administered simultaneously, sequentially or within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, one or more other therapeutic agent and a compound or composition of the invention are administered as a multiple dosage regimen within greater than 24 hours aparts.

In one embodiment, the present invention provides a composition comprising a provided compound or a pharmaceutically acceptable salt thereof and one or more additional therapeutic agents. The therapeutic agent may be administered together with a provided compound or a pharmaceutically acceptable salt thereof, or may be administered prior to or following administration of a provided compound or a pharmaceutically acceptable salt thereof. Suitable therapeutic agents are described in further detail below. In certain embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours before the therapeutic agent. In other embodiments, a provided compound or a pharmaceutically acceptable salt thereof may be administered up to 5 minutes, 10 minutes, 15 minutes, 30 minutes, 1 hour, 2 hours, 3 hours, 4 hours, 5, hours, 6 hours, 7 hours, 8 hours, 9 hours, 10 hours, 11 hours, 12 hours, 13 hours, 14 hours, 15 hours, 16 hours, 17 hours, or 18 hours following the therapeutic agent.

In another embodiment, the present invention provides a method of treating an inflammatory disease, disorder or condition by administering to a patient in need thereof a provided compound or a pharmaceutically acceptable salt thereof and one or more additional therapeutic agents. Such additional therapeutic agents may be small molecules or recombinant biologic agents and include, for example, acetaminophen, non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol, febuxostat (Uloric®), sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), canakinumab (Ilaris®), anti-Jak inhibitors such as tofacitinib, antibodies such as rituximab (Rituxan®), “anti-T-cell” agents such as abatacept (Orencia®), “anti-IL-6” agents such as tocilizumab (Actemra®), diclofenac, cortisone, hyaluronic acid (Synvisc® or Hyalgan®), monoclonal antibodies such as tanezumab, anticoagulants such as heparin (Calcinparine® or Liquaemin®) and warfarin (Coumadin®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot®, anticholinergics or antispasmodics such as dicyclomine (Bentyl®), Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as beclomethasone dipropionate (Beclovent®, Qvar®, and Vanceril®), triamcinolone acetonide (Azmacort®), mometasone (Asthmanex®), budesonide (Pulmocort®), and flunisolide (Aerobid®), Afviar®, Symbicort®, Dulera®, cromolyn sodium (Intal®), methylxanthines such as theophylline (Theo-Dur®, Theolair®, Slo-bid®, Uniphyl®, Theo-24®) and aminophylline, IgE antibodies such as omalizumab (Xolair®), nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), doxorubicin (Hydrodaunorubicin®), vincristine (Oncovin®), bortezomib (Velcade®), and dexamethasone (Decadron ®) in combination with lenalidomide (Revlimid®), or any combination(s) thereof.

In another embodiment, the present invention provides a method of treating gout comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, colchicine (Colcrys®), corticosteroids such as prednisone, prednisolone, methylprednisolone, hydrocortisone, and the like, probenecid, allopurinol and febuxostat (Uloric®).

In another embodiment, the present invention provides a method of treating rheumatoid arthritis comprising administering to a patient in need thereof a provided compound or a pharmaceutically acceptable salt thereof and one or more additional therapeutic agents selected from non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, sulfasalazine (Azulfidine®), antimalarials such as hydroxychloroquine (Plaquenil®) and chloroquine (Aralen®), methotrexate (Rheumatrex®), gold salts such as gold thioglucose (Solganal®), gold thiomalate (Myochrysine®) and auranofin (Ridaura®), D-penicillamine (Depen® or Cuprimine®), azathioprine (Imuran®), cyclophosphamide (Cytoxan®), chlorambucil (Leukeran®), cyclosporine (Sandimmune®), leflunomide (Arava®) and “anti-TNF” agents such as etanercept (Enbrel®), infliximab (Remicade®), golimumab (Simponi®), certolizumab pegol (Cimzia®) and adalimumab (Humira®), “anti-IL-1” agents such as anakinra (Kineret®) and rilonacept (Arcalyst®), antibodies such as rituximab (Rituxan®), “anti-T-cell” agents such as abatacept (Orencia®) and “anti-IL-6” agents such as tocilizumab (Actemra®).

In some embodiments, the present invention provides a method of treating osteoarthritis comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, diclofenac, cortisone, hyaluronic acid (Synvisc® or Hyalgan®) and monoclonal antibodies such as tanezumab.

In some embodiments, the present invention provides a method of treating lupus comprising administering to a patient in need thereof a provided compound or a pharmaceutically acceptable salt thereof and one or more additional therapeutic agents selected from acetaminophen, non-steroidal anti-inflammatory drugs (NSAIDS) such as aspirin, ibuprofen, naproxen, etodolac (Lodine®) and celecoxib, corticosteroids such as prednisone, prednisolone, methylprednisolone, hydrocortisone, and the like, antimalarials such as hydroxychloroquine (Plaquenil®) and chloroquine (Aralen®), cyclophosphamide (Cytoxan®), methotrexate (Rheumatrex®), azathioprine (Imuran®) and anticoagulants such as heparin (Calcinparine® or Liquaemin®) and warfarin (Coumadin®).

In some embodiments, the present invention provides a method of treating inflammatory bowel disease comprising administering to a patient in need thereof a provided compound or a pharmaceutically acceptable salt thereof and one or more additional therapeutic agents selected from mesalamine (Asacol®) sulfasalazine (Azulfidine®), antidiarrheals such as diphenoxylate (Lomotil®) and loperamide (Imodium®), bile acid binding agents such as cholestyramine, alosetron (Lotronex®), lubiprostone (Amitiza®), laxatives such as Milk of Magnesia, polyethylene glycol (MiraLax®), Dulcolax®, Correctol® and Senokot® and anticholinergics or antispasmodics such as dicyclomine (Bentyl®), anti-TNF therapies, steroids, and antibiotics such as Flagyl or ciprofloxacin.

In some embodiments, the present invention provides a method of treating asthma comprising administering to a patient in need thereof a provided compound or a pharmaceutically acceptable salt thereof and one or more additional therapeutic agents selected from Singulair®, beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®, cromolyn sodium (Intal®), methylxanthines such as theophylline (Theo-Dur®, Theolair®, Slo-bid®, Uniphyl®, Theo-24®) and aminophylline, and IgE antibodies such as omalizumab (Xolair®).

In some embodiments, the present invention provides a method of treating COPD comprising administering to a patient in need thereof a provided compound or a pharmaceutically acceptable salt thereof and one or more additional therapeutic agents selected from beta-2 agonists such as albuterol (Ventolin® HFA, Proventil® HFA), levalbuterol (Xopenex®), metaproterenol (Alupent®), pirbuterol acetate (Maxair®), terbutaline sulfate (Brethaire®), salmeterol xinafoate (Serevent®) and formoterol (Foradil®), anticholinergic agents such as ipratropium bromide (Atrovent®) and tiotropium (Spiriva®), methylxanthines such as theophylline (Theo-Dur®, Theolair®, Slo-bid®, Uniphyl®, Theo-24®) and aminophylline, inhaled corticosteroids such as prednisone, prednisolone, beclomethasone dipropionate (Beclovent®, Qvar®, and Vanceril®), triamcinolone acetonide (Azmacort®), mometasone (Asthmanex®), budesonide (Pulmocort®), flunisolide (Aerobid®), Afviar®, Symbicort®, and Dulera®,

In some embodiments, the present invention provides a method of treating HIV comprising administering to a patient in need thereof a provided compound or a pharmaceutically acceptable salt thereof and one or more additional therapeutic agents selected from nucleoside reverse transcriptase inhibitors such as zidovudine (Retrovir®), abacavir (Ziagen®), abacavir/lamivudine (Epzicom®), abacavir/lamivudine/zidovudine (Trizivir®), didanosine (Videx®), emtricitabine (Emtriva®), lamivudine (Epivir®), lamivudine/zidovudine (Combivir®), stavudine (Zerit®), and zalcitabine (Hivid®), non-nucleoside reverse transcriptase inhibitors such as delavirdine (Rescriptor®), efavirenz (Sustiva®), nevairapine (Viramune®) and etravirine (Intelence®), nucleotide reverse transcriptase inhibitors such as tenofovir (Viread®), protease inhibitors such as amprenavir (Agenerase®), atazanavir (Reyataz®), darunavir (Prezista®), fosamprenavir (Lexiva®), indinavir (Crixivan®), lopinavir and ritonavir (Kaletra®), nelfinavir (Viracept®), ritonavir (Norvir®), saquinavir (Fortovase® or Invirase®), and tipranavir (Aptivus®), entry inhibitors such as enfuvirtide (Fuzeon®) and maraviroc (Selzentry®), integrase inhibitors such as raltegravir (Isentress®), and combinations thereof.

In another embodiment, the present invention provides a method of treating a hematological malignancy comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a solid tumor comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, a BTK inhibitor, a JAK/pan-JAK inhibitor, a TYK2 inhibitor, a PI3K inhibitor, a SYK inhibitor, and combinations thereof.

In another embodiment, the present invention provides a method of treating a hematological malignancy comprising administering to a patient in need thereof a provided compound or a pharmaceutically acceptable salt thereof and a Hedgehog (Hh) signaling pathway inhibitor. In some embodiments, the hematological malignancy is DLBCL (Ramirez et al “Defining causative factors contributing in the activation of hedgehog signaling in diffuse large B-cell lymphoma” Leuk. Res. (2012), published online July 17, and incorporated herein by reference in its entirety).

In another embodiment, the present invention provides a method of treating diffuse large B-cell lymphoma (DLBCL) comprising administering to a patient in need thereof a provided compound or a pharmaceutically acceptable salt thereof and one or more additional therapeutic agents selected from rituximab (Rituxan®), cyclophosphamide (Cytoxan®), doxorubicin (Hydrodaunorubicin®), vincristine (Oncovin®), prednisone, a hedgehog signaling inhibitor, and combinations thereof.

In some embodiments, the present invention provides a method of treating DLBCL comprising administering to a patient in need thereof a provided compound or a pharmaceutically acceptable salt thereof and a CHOP (cyclophosphamide, Hydrodaunorubicin®, Oncovin®, and prednisone or prednisolone) or R-CHOP (rituximab, cyclophosphamide, Hydrodaunorubicin®, Oncovin®, and prednisone or prednisolone) chemotherapy regimen.

In some embodiments, the present invention provides a method of treating DLBCL comprising administering to a patient in need thereof a provided compound or a pharmaceutically acceptable salt thereof and a rituximab/bendamustine chemotherapy regimen.

In some embodiments, the present invention provides a method of treating DLBCL comprising administering to a patient in need thereof a provided compound or a pharmaceutically acceptable salt thereof and a BTK inhibitor (e.g., ibrutinib).

In some embodiments, the present invention provides a method of treating DLBCL comprising administering to a patient in need thereof a provided compound or a pharmaceutically acceptable salt thereof and an anti-CD20 antibody (e.g., rituximab).

In some embodiments, the present invention provides a method of treating DLBCL comprising administering to a patient in need thereof a provided compound or a pharmaceutically acceptable salt thereof and an anti-CD79B ADC (e.g., polatuzumab).

In some embodiments, the present invention provides a method of treating DLBCL comprising administering to a patient in need thereof a provided compound or a pharmaceutically acceptable salt thereof and a BCL2 inhibitor (e.g., venetoclax).

In some embodiments, the present invention provides a method of treating DLBCL comprising administering to a patient in need thereof a provided compound or a pharmaceutically acceptable salt thereof and lenalidomide

In some embodiments, the present invention provides a method of treating DLBCL comprising administering to a patient in need thereof a provided compound or a pharmaceutically acceptable salt thereof and a PI3K inhibitor (e.g., umbralisib).

In some embodiments, the present invention provides a method of treating a T-cell disease or deficiency describing herein comprising administering to a patient in need thereof a provided compound or a pharmaceutically acceptable salt thereof and a PI3K inhibitor (e.g., umbralisib).

In some embodiments, the present invention provides a method of treating DLBCL comprising administering to a patient in need thereof a provided compound or a pharmaceutically acceptable salt thereof and a protesome inhibitor (e.g., bortezomib)

In some embodiments, the present invention provides a method of treating a T-cell disease or deficiency describing herein comprising administering to a patient in need thereof a provided compound or a pharmaceutically acceptable salt thereof and a protesome inhibitor (e.g., bortezomib).

In another embodiment, the present invention provides a method of treating multiple myeloma comprising administering to a patient in need thereof a provided compound or a pharmaceutically acceptable salt thereof and one or more additional therapeutic agents selected from bortezomib (Velcade®), and dexamethasone (Decadron®), a hedgehog signaling inhibitor, a BTK inhibitor, a JAK/pan-JAK inhibitor, a TYK2 inhibitor, a PI3K inhibitor, a SYK inhibitor in combination with lenalidomide (Revlimid®).

In another embodiment, the present invention provides a method of treating Waldenström's macroglobulinemia comprising administering to a patient in need thereof a provided compound or a pharmaceutically acceptable salt thereof and one or more additional therapeutic agents selected from chlorambucil (Leukeran®), cyclophosphamide (Cytoxan®, Neosar®), fludarabine (Fludara®), cladribine (Leustatin®), rituximab (Rituxan®), a hedgehog signaling inhibitor, a BTK inhibitor, a JAK/pan-JAK inhibitor, a TYK2 inhibitor, a PI3K inhibitor, and a SYK inhibitor.

In some embodiments, one or more other therapeutic agent is an antagonist of the hedgehog pathway. Approved hedgehog pathway inhibitors which may be used in the present invention include sonidegib (Odomzo®, Sun Pharmaceuticals); and vismodegib (Erivedge®, Genentech), both for treatment of basal cell carcinoma.

In some embodiments, one or more other therapeutic agent is a Poly ADP ribose polymerase (PARP) inhibitor. In some embodiments, a PARP inhibitor is selected from olaparib (Lynparza®, AstraZeneca); rucaparib (Rubraca®, Clovis Oncology); niraparib (Zejula®, Tesaro); talazoparib (MDV3800/BMN 673/LT00673, Medivation/Pfizer/Biomarin); veliparib (ABT-888, AbbVie); and BGB-290 (BeiGene, Inc.).

In some embodiments, one or more other therapeutic agent is a histone deacetylase (HDAC) inhibitor. In some embodiments, an HDAC inhibitor is selected from vorinostat (Zolinza®, Merck); romidepsin (Istodax®, Celgene); panobinostat (Farydak®, Novartis); belinostat (Beleodaq®, Spectrum Pharmaceuticals); entinostat (SNDX-275, Syndax Pharmaceuticals) (NCT00866333); and chidamide (Epidaza®, HBI-8000, Chipscreen Biosciences, China).

In some embodiments, one or more other therapeutic agent is a CDK inhibitor, such as a CDK4/CDK6 inhibitor. In some embodiments, a CDK 4/6 inhibitor is selected from palbociclib (Ibrance®, Pfizer); ribociclib (Kisqali®, Novartis); abemaciclib (Ly2835219, Eli Lilly); and trilaciclib (G1T28, G1 Therapeutics).

In some embodiments, one or more other therapeutic agent is a folic acid inhibitor. Approved folic acid inhibitors useful in the present invention include pemetrexed (Alimta®, Eli Lilly).

In some embodiments, one or more other therapeutic agent is a CC chemokine receptor 4 (CCR4) inhibitor. CCR4 inhibitors being studied that may be useful in the present invention include mogamulizumab (Poteligeo®, Kyowa Hakko Kirin, Japan).

In some embodiments, one or more other therapeutic agent is an isocitrate dehydrogenase (IDH) inhibitor. IDH inhibitors being studied which may be used in the present invention include AG120 (Celgene; NCT02677922); AG221 (Celgene, NCT02677922; NCT02577406); BAY1436032 (Bayer, NCT02746081); IDH305 (Novartis, NCT02987010).

In some embodiments, one or more other therapeutic agent is an arginase inhibitor. Arginase inhibitors being studied which may be used in the present invention include AEB1102 (pegylated recombinant arginase, Aeglea Biotherapeutics), which is being studied in Phase 1 clinical trials for acute myeloid leukemia and myelodysplastic syndrome (NCT02732184) and solid tumors (NCT02561234); and CB-1158 (Calithera Biosciences).

In some embodiments, one or more other therapeutic agent is a glutaminase inhibitor. Glutaminase inhibitors being studied which may be used in the present invention include CB-839 (Calithera Biosciences).

In some embodiments, one or more other therapeutic agent is an antibody that binds to tumor antigens, that is, proteins expressed on the cell surface of tumor cells. Approved antibodies that bind to tumor antigens which may be used in the present invention include rituximab (Rituxan®, Genentech/BiogenIdec); ofatumumab (anti-CD20, Arzerra®, GlaxoSmithKline); obinutuzumab (anti-CD20, Gazyva®, Genentech), ibritumomab (anti-CD20 and Yttrium-90, Zevalin®, Spectrum Pharmaceuticals); daratumumab (anti-CD38, Darzal ex®, Janssen Biotech), dinutuximab (anti-glycolipid GD2, Unituxin®, United Therapeutics); trastuzumab (anti-HER2, Herceptin®, Genentech); ado-trastuzumab emtansine (anti-HER2, fused to emtansine, Kadcyla®, Genentech); and pertuzumab (anti-HER2, Perjeta®, Genentech); and brentuximab vedotin (anti-CD30-drug conjugate, Adcetris®, Seattle Genetics).

In some embodiments, one or more other therapeutic agent is a topoisomerase inhibitor. Approved topoisomerase inhibitors useful in the present invention include irinotecan (Onivyde®, Merrimack Pharmaceuticals); topotecan (Hycamtin®, GlaxoSmithKline). Topoisomerase inhibitors being studied which may be used in the present invention include pixantrone (Pixuvri®, CTI Biopharma).

In some embodiments, one or more other therapeutic agent is an inhibitor of anti-apoptotic proteins, such as BCL-2. Approved anti-apoptotics which may be used in the present invention include venetoclax (Venclexta®, AbbVie/Genentech); and blinatumomab (Blincyto®, Amgen). Other therapeutic agents targeting apoptotic proteins which have undergone clinical testing and may be used in the present invention include navitoclax (ABT-263, Abbott), a BCL-2 inhibitor (NCT02079740).

In some embodiments, one or more other therapeutic agent is an androgen receptor inhibitor. Approved androgen receptor inhibitors useful in the present invention include enzalutamide (Xtandi®, Astellas/Medivation); approved inhibitors of androgen synthesis include abiraterone (Zytiga®, Centocor/Ortho); approved antagonist of gonadotropin-releasing hormone (GnRH) receptor (degaralix, Firmagon®, Ferring Pharmaceuticals).

In some embodiments, one or more other therapeutic agent is a selective estrogen receptor modulator (SERM), which interferes with the synthesis or activity of estrogens. Approved SERMs useful in the present invention include raloxifene (Evista®, Eli Lilly).

In some embodiments, one or more other therapeutic agent is an inhibitor of bone resorption. An approved therapeutic which inhibits bone resorption is Denosumab (Xgeva®, Amgen), an antibody that binds to RANKL, prevents binding to its receptor RANK, found on the surface of osteoclasts, their precursors, and osteoclast-like giant cells, which mediates bone pathology in solid tumors with osseous metastases. Other approved therapeutics that inhibit bone resorption include bisphosphonates, such as zoledronic acid (Zometa®, Novartis).

In some embodiments, one or more other therapeutic agent is an inhibitor of interaction between the two primary p53 suppressor proteins, MDMX and MDM2. Inhibitors of p53 suppression proteins being studied which may be used in the present invention include ALRN-6924 (Aileron), a stapled peptide that equipotently binds to and disrupts the interaction of MDMX and MDM2 with p53. ALRN-6924 is currently being evaluated in clinical trials for the treatment of AML, advanced myelodysplastic syndrome (MDS) and peripheral T-cell lymphoma (PTCL) (NCT02909972; NCT02264613).

In some embodiments, one or more other therapeutic agent is an inhibitor of transforming growth factor-beta (TGF-beta or TGFB). Inhibitors of TGF-beta proteins being studied which may be used in the present invention include NIS793 (Novartis), an anti-TGF-beta antibody being tested in the clinic for treatment of various cancers, including breast, lung, hepatocellular, colorectal, pancreatic, prostate and renal cancer (NCT 02947165). In some embodiments, the inhibitor of TGF-beta proteins is fresolimumab (GC1008; Sanofi-Genzyme), which is being studied for melanoma (NCT00923169); renal cell carcinoma (NCT00356460); and non-small cell lung cancer (NCT02581787). Additionally, in some embodiments, the additional therapeutic agent is a TGF-beta trap, such as described in Connolly et al. (2012) Int'l J. Biological Sciences 8:964-978. One therapeutic compound currently in clinical trials for treatment of solid tumors is M7824 (Merck KgaA—formerly MSB0011459X), which is a bispecific, anti-PD-L1/TGFB trap compound (NCT02699515); and (NCT02517398). M7824 is comprised of a fully human IgG1 antibody against PD-L1 fused to the extracellular domain of human TGF-beta receptor II, which functions as a TGFB “trap.”

In some embodiments, one or more other therapeutic agent is selected from glembatumumab vedotin-monomethyl auristatin E (MMAE) (Celldex), an anti-glycoprotein NMB (gpNMB) antibody (CR011) linked to the cytotoxic MMAE. gpNMB is a protein overexpressed by multiple tumor types associated with cancer cells' ability to metastasize.

In some embodiments, one or more other therapeutic agent is an antiproliferative compound. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZd₆244 from AstraZeneca, PD181461 from Pfizer and leucovorin.

In some embodiments, the present invention provides a method of treating Alzheimer's disease comprising administering to a patient in need thereof a provided compound or a pharmaceutically acceptable salt thereof and one or more additional therapeutic agents selected from donepezil (Aricept®), rivastigmine (Excelon®), galantamine (Razadyne®), tacrine (Cognex®), and memantine (Namenda®).

In some embodiments, one or more other therapeutic agent is a taxane compound, which causes disruption of microtubules, which are essential for cell division. In some embodiments, a taxane compound is selected from paclitaxel (Taxol®, Bristol-Myers Squibb), docetaxel (Taxotere®, Sanofi-Aventis; Docefrez®, Sun Pharmaceutical), albumin-bound paclitaxel (Abraxane®; Abraxis/Celgene), cabazitaxel (Jevtana®, Sanofi-Aventis), and SID530 (SK Chemicals, Co.) (NCT00931008).

In some embodiments, one or more other therapeutic agent is a nucleoside inhibitor, or a therapeutic agent that interferes with normal DNA synthesis, protein synthesis, cell replication, or will otherwise inhibit rapidly proliferating cells.

In some embodiments, a nucleoside inhibitor is selected from trabectedin (guanidine alkylating agent, Yondelis®, Janssen Oncology), mechlorethamine (alkylating agent, Valchlor®, Aktelion Pharmaceuticals); vincristine (Oncovin®, Eli Lilly; Vincasar®, Teva Pharmaceuticals; Marqibo®, Talon Therapeutics); temozolomide (prodrug to alkylating agent 5-(3-methyltriazen-1-yl)-imidazole-4-carboxamide (MTIC) Temodar®, Merck); cytarabine injection (ara-C, antimetabolic cytidine analog, Pfizer); lomustine (alkylating agent, CeeNU®, Bristol-Myers Squibb; Gleostine®, NextSource Biotechnology); azacitidine (pyrimidine nucleoside analog of cytidine, Vidaza®, Celgene); omacetaxine mepesuccinate (cephalotaxine ester) (protein synthesis inhibitor, Synribo®; Teva Pharmaceuticals); asparaginase Envinia chrysanthemi (enzyme for depletion of asparagine, Elspar®, Lundbeck; Erwinaze®, EUSA Pharma); eribulin mesylate (microtubule inhibitor, tubulin-based antimitotic, Halaven®, Eisai); cabazitaxel (microtubule inhibitor, tubulin-based antimitotic, Jevtana®, Sanofi-Aventis); capacetrine (thymidylate synthase inhibitor, Xeloda®, Genentech); bendamustine (bifunctional mechlorethamine derivative, believed to form interstrand DNA cross-links, Treanda®, Cephalon/Teva); ixabepilone (semi-synthetic analog of epothilone B, microtubule inhibitor, tubulin-based antimitotic, Ixempra®, Bristol-Myers Squibb); nelarabine (prodrug of deoxyguanosine analog, nucleoside metabolic inhibitor, Arranon®, Novartis); clorafabine (prodrug of ribonucleotide reductase inhibitor, competitive inhibitor of deoxycytidine, Clolar®, Sanofi-Aventis); and trifluridine and tipiracil (thymidine-based nucleoside analog and thymidine phosphorylase inhibitor, Lonsurf®, Taiho Oncology).

In some embodiments, one or more other therapeutic agent is a kinase inhibitor or VEGF-R antagonist. Approved VEGF inhibitors and kinase inhibitors useful in the present invention include: bevacizumab (Avastin®, Genentech/Roche) an anti-VEGF monoclonal antibody; ramucirumab (Cyramza®, Eli Lilly), an anti-VEGFR-2 antibody and ziv-aflibercept, also known as VEGF Trap (Zaltrap®; Regeneron/Sanofi). VEGFR inhibitors, such as regorafenib (Stivarga®, Bayer); vandetanib (Caprelsa®, AstraZeneca); axitinib (Inlyta®, Pfizer); and lenvatinib (Lenvima®, Eisai); Raf inhibitors, such as sorafenib (Nexavar®, Bayer AG and Onyx); dabrafenib (Tafinlar®, Novartis); and vemurafenib (Zelboraf®, Genentech/Roche); MEK inhibitors, such as cobimetanib (Cotellic®, Exelexis/Genentech/Roche); trametinib (Mekinist®, Novartis); Bcr-Abl tyrosine kinase inhibitors, such as imatinib (Gleevec®, Novartis); nilotinib (Tasigna®, Novartis); dasatinib (Sprycel®, BristolMyersSquibb); bosutinib (Bosulif®, Pfizer); and ponatinib (Inclusig®, Ariad Pharmaceuticals); Her2 and EGFR inhibitors, such as gefitinib (Iressa®, AstraZeneca); erlotinib (Tarceeva®, Genentech/Roche/Astellas); lapatinib (Tykerb®, Novartis); afatinib (Gilotrif®, Boehringer Ingelheim); osimertinib (targeting activated EGFR, Tagrisso®, AstraZeneca); and brigatinib (Alunbrig®, Ariad Pharmaceuticals); c-Met and VEGFR2 inhibitors, such as cabozanitib (Cometriq®, Exelexis); and multikinase inhibitors, such as sunitinib (Sutent®, Pfizer); pazopanib (Votrient®, Novartis); ALK inhibitors, such as crizotinib (Xalkori®, Pfizer); ceritinib (Zykadia®, Novartis); and alectinib (Alecenza®, Genentech/Roche); Bruton's tyrosine kinase inhibitors, such as ibrutinib (Imbruvica®, Pharmacyclics/Janssen); and Flt3 receptor inhibitors, such as midostaurin (Rydapt®, Novartis).

Other kinase inhibitors and VEGF-R antagonists that are in development and may be used in the present invention include tivozanib (Aveo Pharmaecuticals); vatalanib (Bayer/Novartis); lucitanib (Clovis Oncology); dovitinib (TKI258, Novartis); Chiauanib (Chipscreen Biosciences); CEP-11981 (Cephalon); linifanib (Abbott Laboratories); neratinib (HKI-272, Puma Biotechnology); radotinib (Supect®, IY5511, Il-Yang Pharmaceuticals, S. Korea); ruxolitinib (Jakafi®, Incyte Corporation); PTC299 (PTC Therapeutics); CP-547,632 (Pfizer); foretinib (Exelexis, GlaxoSmithKline); quizartinib (Daiichi Sankyo) and motesanib (Amgen/Takeda).

In another embodiment, the present invention provides a method of treating organ transplant rejection or graft vs. host disease comprising administering to a patient in need thereof a provided compound or a pharmaceutically acceptable salt thereof and one or more additional therapeutic agents selected from a steroid, cyclosporin, FK506, rapamycin, a hedgehog signaling inhibitor, a BTK inhibitor, a JAK/pan-JAK inhibitor, a TYK2 inhibitor, a PI3K inhibitor, and a SYK inhibitor.

In another embodiment, the present invention provides a method of treating or lessening the severity of a disease comprising administering to a patient in need thereof a provided compound or a pharmaceutically acceptable salt thereof and a BTK inhibitor, wherein the disease is selected from inflammatory bowel disease, arthritis, systemic lupus erythematosus (SLE), vasculitis, idiopathic thrombocytopenic purpura (ITP), rheumatoid arthritis, psoriatic arthritis, osteoarthritis, Still's disease, juvenile arthritis, diabetes, myasthenia gravis, Hashimoto's thyroiditis, Ord's thyroiditis, Graves' disease, autoimmune thyroiditis, Sjogren's syndrome, multiple sclerosis, systemic sclerosis, Lyme neuroborreliosis, Guillain-Barre syndrome, acute disseminated encephalomyelitis, Addison's disease, opsoclonus-myoclonus syndrome, ankylosing spondylosis, antiphospholipid antibody syndrome, aplastic anemia, autoimmune hepatitis, autoimmune gastritis, pernicious anemia, celiac disease, Goodpasture's syndrome, idiopathic thrombocytopenic purpura, optic neuritis, scleroderma, primary biliary cirrhosis, Reiter's syndrome, Takayasu's arteritis, temporal arteritis, warm autoimmune hemolytic anemia, Wegener's granulomatosis, psoriasis, alopecia universalis, Behcet's disease, chronic fatigue, dysautonomia, membranous glomerulonephropathy, endometriosis, interstitial cystitis, pemphigus vulgaris, bullous pemphigoid, neuromyotonia, scleroderma, vulvodynia, a hyperproliferative disease, rejection of transplanted organs or tissues, Acquired Immunodeficiency Syndrome (AIDS, also known as HIV), type 1 diabetes, graft versus host disease, transplantation, transfusion, anaphylaxis, allergies (e.g., allergies to plant pollens, latex, drugs, foods, insect poisons, animal hair, animal dander, dust mites, or cockroach calyx), type I hypersensitivity, allergic conjunctivitis, allergic rhinitis, and atopic dermatitis, asthma, appendicitis, atopic dermatitis, asthma, allergy, blepharitis, bronchiolitis, bronchitis, bursitis, cervicitis, cholangitis, cholecystitis, chronic graft rejection, colitis, conjunctivitis, Crohn's disease, cystitis, dacryoadenitis, dermatitis, dermatomyositis, encephalitis, endocarditis, endometritis, enteritis, enterocolitis, epicondylitis, epididymitis, fasciitis, fibrositis, gastritis, gastroenteritis, Henoch-Schonlein purpura, hepatitis, hidradenitis suppurativa, immunoglobulin A nephropathy, interstitial lung disease, laryngitis, mastitis, meningitis, myelitis myocarditis, myositis, nephritis, oophoritis, orchitis, osteitis, otitis, pancreatitis, parotitis, pericarditis, peritonitis, pharyngitis, pleuritis, phlebitis, pneumonitis, pneumonia, polymyositis, proctitis, prostatitis, pyelonephritis, rhinitis, salpingitis, sinusitis, stomatitis, synovitis, tendonitis, tonsillitis, ulcerative colitis, uveitis, vaginitis, vasculitis, or vulvitis, B-cell proliferative disorder, e.g., diffuse large B cell lymphoma, follicular lymphoma, chronic lymphocytic lymphoma, chronic lymphocytic leukemia, acute lymphocytic leukemia, B-cell prolymphocytic leukemia, lymphoplasmacytic lymphoma/Waldenstrom macroglobulinemia, splenic marginal zone lymphoma, multiple myeloma (also known as plasma cell myeloma), non-Hodgkin's lymphoma, Hodgkin's lymphoma, plasmacytoma, extranodal marginal zone B cell lymphoma, nodal marginal zone B cell lymphoma, mantle cell lymphoma, mediastinal (thymic) large B cell lymphoma, intravascular large B cell lymphoma, primary effusion lymphoma, Burkitt lymphoma/leukemia, or lymphomatoid granulomatosis, breast cancer, prostate cancer, or cancer of the mast cells (e.g., mastocytoma, mast cell leukemia, mast cell sarcoma, systemic mastocytosis), bone cancer, colorectal cancer, pancreatic cancer, diseases of the bone and joints including, without limitation, rheumatoid arthritis, seronegative spondyloarthropathies (including ankylosing spondylitis, psoriatic arthritis and Reiter's disease), Behcet's disease, Sjogren's syndrome, systemic sclerosis, osteoporosis, bone cancer, bone metastasis, a thromboembolic disorder, (e.g., myocardial infarct, angina pectoris, reocclusion after angioplasty, restenosis after angioplasty, reocclusion after aortocoronary bypass, restenosis after aortocoronary bypass, stroke, transitory ischemia, a peripheral arterial occlusive disorder, pulmonary embolism, deep venous thrombosis), inflammatory pelvic disease, urethritis, skin sunburn, sinusitis, pneumonitis, encephalitis, meningitis, myocarditis, nephritis, osteomyelitis, myositis, hepatitis, gastritis, enteritis, dermatitis, gingivitis, appendicitis, pancreatitis, cholocystitus, agammaglobulinemia, psoriasis, allergy, Crohn's disease, irritable bowel syndrome, ulcerative colitis, Sjogren's disease, tissue graft rejection, hyperacute rejection of transplanted organs, asthma, allergic rhinitis, chronic obstructive pulmonary disease (COPD), autoimmune polyglandular disease (also known as autoimmune polyglandular syndrome), autoimmune alopecia, pernicious anemia, glomerulonephritis, dermatomyositis, multiple sclerosis, scleroderma, vasculitis, autoimmune hemolytic and thrombocytopenic states, Goodpasture's syndrome, atherosclerosis, Addison's disease, Parkinson's disease, Alzheimer's disease, diabetes, septic shock, systemic lupus erythematosus (SLE), rheumatoid arthritis, psoriatic arthritis, juvenile arthritis, osteoarthritis, chronic idiopathic thrombocytopenic purpura, Waldenstrom macroglobulinemia, myasthenia gravis, Hashimoto's thyroiditis, atopic dermatitis, degenerative joint disease, vitiligo, autoimmune hypopituitarism, Guillain-Barre syndrome, Behcet's disease, scleraderma, mycosis fungoides, acute inflammatory responses (such as acute respiratory distress syndrome and ischemia/reperfusion injury), and Graves' disease.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from a cancer, a neurodegenative disorder, an angiogenic disorder, a viral disease, an autoimmune disease, an inflammatory disorder, a hormone-related disease, conditions associated with organ transplantation, immunodeficiency disorders, a destructive bone disorder, a proliferative disorder, an infectious disease, a condition associated with cell death, thrombin-induced platelet aggregation, chronic myelogenous leukemia (CML), chronic lymphocytic leukemia (CLL), liver disease, pathologic immune conditions involving T cell activation, a cardiovascular disorder, and a CNS disorder.

In another embodiment, the present invention provides a method of treating or lessening the severity of a disease comprising administering to a patient in need thereof a provided compound or a pharmaceutically acceptable salt thereof and a PI3K inhibitor, wherein the disease is selected from benign or malignant tumor, carcinoma or solid tumor of the brain, kidney (e.g., renal cell carcinoma (RCC)), liver, adrenal gland, bladder, breast, stomach, gastric tumors, ovaries, colon, rectum, prostate, pancreas, lung, vagina, endometrium, cervix, testis, genitourinary tract, esophagus, larynx, skin, bone or thyroid, sarcoma, glioblastomas, neuroblastomas, multiple myeloma or gastrointestinal cancer, especially colon carcinoma or colorectal adenoma or a tumor of the neck and head, an epidermal hyperproliferation, psoriasis, prostate hyperplasia, a neoplasia, a neoplasia of epithelial character, adenoma, adenocarcinoma, keratoacanthoma, epidermoid carcinoma, large cell carcinoma, non-small-cell lung carcinoma, lymphomas, (including, for example, non-Hodgkin's Lymphoma (NHL) and Hodgkin's lymphoma (also termed Hodgkin's or Hodgkin's disease)), a mammary carcinoma, follicular carcinoma, undifferentiated carcinoma, papillary carcinoma, seminoma, melanoma, or a leukemia, diseases include Cowden syndrome, Lhermitte-Dudos disease and Bannayan-Zonana syndrome, or diseases in which the PI3K/PKB pathway is aberrantly activated, asthma of whatever type or genesis including both intrinsic (non-allergic) asthma and extrinsic (allergic) asthma, mild asthma, moderate asthma, severe asthma, bronchitic asthma, exercise-induced asthma, occupational asthma and asthma induced following bacterial infection, acute lung injury (ALI), adult/acute respiratory distress syndrome (ARDS), chronic obstructive pulmonary, airways or lung disease (COPD, COAD or COLD), including chronic bronchitis or dyspnea associated therewith, emphysema, as well as exacerbation of airways hyperreactivity consequent to other drug therapy, in particular other inhaled drug therapy, bronchitis of whatever type or genesis including, but not limited to, acute, arachidic, catarrhal, croupus, chronic or phthinoid bronchitis, pneumoconiosis (an inflammatory, commonly occupational, disease of the lungs, frequently accompanied by airways obstruction, whether chronic or acute, and occasioned by repeated inhalation of dusts) of whatever type or genesis, including, for example, aluminosis, anthracosis, asbestosis, chalicosis, ptilosis, siderosis, silicosis, tabacosis and byssinosis, Loffler's syndrome, eosinophilic, pneumonia, parasitic (in particular metazoan) infestation (including tropical eosinophilia), bronchopulmonary aspergillosis, polyarteritis nodosa (including Churg-Strauss syndrome), eosinophilic granuloma and eosinophil-related disorders affecting the airways occasioned by drug-reaction, psoriasis, contact dermatitis, atopic dermatitis, alopecia areata, erythema multiforma, dermatitis herpetiformis, scleroderma, vitiligo, hypersensitivity angiitis, urticaria, bullous pemphigoid, lupus erythematosus, pemphisus, epidermolysis bullosa acquisita, conjunctivitis, keratoconjunctivitis sicca, and vernal conjunctivitis, diseases affecting the nose including allergic rhinitis, and inflammatory disease in which autoimmune reactions are implicated or having an autoimmune component or etiology, including autoimmune hematological disorders (e.g. hemolytic anemia, aplastic anemia, pure red cell anemia and idiopathic thrombocytopenia), systemic lupus erythematosus, rheumatoid arthritis, polychondritis, sclerodoma, Wegener granulamatosis, dermatomyositis, chronic active hepatitis, myasthenia gravis, Steven-Johnson syndrome, idiopathic sprue, autoimmune inflammatory bowel disease (e.g. ulcerative colitis and Crohn's disease), endocrine opthalmopathy, Grave's disease, sarcoidosis, alveolitis, chronic hypersensitivity pneumonitis, multiple sclerosis, primary biliary cirrhosis, uveitis (anterior and posterior), keratoconjunctivitis sicca and vernal keratoconjunctivitis, interstitial lung fibrosis, psoriatic arthritis and glomerulonephritis (with and without nephrotic syndrome, e.g. including idiopathic nephrotic syndrome or minal change nephropathy, restenosis, cardiomegaly, atherosclerosis, myocardial infarction, ischemic stroke and congestive heart failure, Alzheimer's disease, Parkinson's disease, amyotrophic lateral sclerosis, Huntington's disease, and cerebral ischemia, and neurodegenerative disease caused by traumatic injury, glutamate neurotoxicity and hypoxia.

In some embodiments, one or more other therapeutic agent is a phosphatidylinositol 3 kinase (PI3K) inhibitor. In some embodiments, a PI3K inhibitor is selected from idelalisib (Zydelig®, Gilead), alpelisib (BYL719, Novartis), taselisib (GDC-0032, Genentech/Roche); pictilisib (GDC-0941, Genentech/Roche); copanlisib (BAY806946, Bayer); duvelisib (formerly IPI-145, Infinity Pharmaceuticals); PQR309 (Piqur Therapeutics, Switzerland); and TGR1202 (formerly RP5230, TG Therapeutics).

The compounds and compositions, according to the method of the present invention, may be administered using any amount and any route of administration effective for treating or lessening the severity of a cancer, an autoimmune disorder, a proliferative disorder, an inflammatory disorder, a neurodegenerative or neurological disorder, schizophrenia, a bone-related disorder, liver disease, or a cardiac disorder. The exact amount required will vary from subject to subject, depending on the species, age, and general condition of the subject, the severity of the infection, the particular agent, its mode of administration, and the like. Compounds of the invention are preferably formulated in dosage unit form for ease of administration and uniformity of dosage. The expression “dosage unit form” as used herein refers to a physically discrete unit of agent appropriate for the patient to be treated. It will be understood, however, that the total daily usage of the compounds and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the infection being treated. In certain embodiments, the compounds of the invention may be administered orally or parenterally at dosage levels of about 0.01 mg/kg to about 50 mg/kg and preferably from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

According to one embodiment, the invention relates to a method of inhibiting protein kinase activity or degading a protein kinase in a biological sample comprising the step of contacting said biological sample with a compound of this invention, or a composition comprising said compound.

According to another embodiment, the invention relates to a method of inhibiting or degrading a member of the TAM receptor kinase family (i.e., Tyro3, Axl, and Mer) or a mutant thereof, activity in a biological sample comprising the step of contacting said biological sample with a compound of this invention, or a composition comprising said compound.

The term “biological sample”, as used herein, includes, without limitation, cell cultures or extracts thereof biopsied material obtained from a mammal or extracts thereof; and blood, saliva, urine, feces, semen, tears, or other body fluids or extracts thereof

Inhibition and/or degradation of a protein kinase, or a protein kinase selected from a member of the TAM receptor kinase family (i.e., Tyro3, Axl, and Mer), or a mutant thereof, activity in a biological sample is useful for a variety of purposes that are known to one of skill in the art. Examples of such purposes include, but are not limited to, blood transfusion, organ-transplantation, biological specimen storage, and biological assays.

Another embodiment of the present invention relates to a method of degrading a protein kinase and/or inhibiting protein kinase activity in a patient comprising the step of administering to said patient a compound of the present invention, or a composition comprising said compound.

According to another embodiment, the invention relates to a method of degrading and/or inhibiting one or more of a member of the TAM receptor kinase family (i.e., Tyro3, Axl, and Mer), or a mutant thereof, activity in a patient comprising the step of administering to said patient a compound of the present invention, or a composition comprising said compound. In other embodiments, the present invention provides a method for treating a disorder mediated by one or more of a member of the TAM receptor kinase family (i.e., Tyro3, Axl, and Mer), or a mutant thereof, in a patient in need thereof, comprising the step of administering to said patient a compound according to the present invention or pharmaceutically acceptable composition thereof. Such disorders are described in detail herein.

Depending upon the particular condition, or disease, to be treated, additional therapeutic agents that are normally administered to treat that condition, may also be present in the compositions of this invention. As used herein, additional therapeutic agents that are normally administered to treat a particular disease, or condition, are known as “appropriate for the disease, or condition, being treated.”

A compound of the current invention may also be used to advantage in combination with other antiproliferative compounds. Such antiproliferative compounds include, but are not limited to aromatase inhibitors; antiestrogens; topoisomerase I inhibitors; topoisomerase II inhibitors; microtubule active compounds; alkylating compounds; histone deacetylase inhibitors; compounds which induce cell differentiation processes; cyclooxygenase inhibitors; MMP inhibitors; mTOR inhibitors; antineoplastic antimetabolites; platin compounds; compounds targeting/decreasing a protein or lipid kinase activity and further anti-angiogenic compounds; compounds which target, decrease or inhibit the activity of a protein or lipid phosphatase; gonadorelin agonists; anti-androgens; methionine aminopeptidase inhibitors; matrix metalloproteinase inhibitors; bisphosphonates; biological response modifiers; antiproliferative antibodies; heparanase inhibitors; inhibitors of Ras oncogenic isoforms; telomerase inhibitors; proteasome inhibitors; compounds used in the treatment of hematologic malignancies; compounds which target, decrease or inhibit the activity of Flt-3; Hsp90 inhibitors such as 17-AAG (17-allylaminogeldanamycin, NSC330507), 17-DMAG (17-dimethylaminoethylamino-17-demethoxy-geldanamycin, NSC707545), IPI-504, CNF1010, CNF2024, CNF1010 from Conforma Therapeutics; temozolomide (Temodal®); kinesin spindle protein inhibitors, such as SB715992 or SB743921 from GlaxoSmithKline, or pentamidine/chlorpromazine from CombinatoRx; MEK inhibitors such as ARRY142886 from Array BioPharma, AZD6244 from AstraZeneca, PD181461 from Pfizer and leucovorin.

The term “aromatase inhibitor” as used herein relates to a compound which inhibits estrogen production, for instance, the conversion of the substrates androstenedione and testosterone to estrone and estradiol, respectively. The term includes, but is not limited to steroids, especially atamestane, exemestane and formestane and, in particular, non-steroids, especially aminoglutethimide, roglethimide, pyridoglutethimide, trilostane, testolactone, ketokonazole, vorozole, fadrozole, anastrozole and letrozole. Exemestane is marketed under the trade name Aromasin™. Formestane is marketed under the trade name Lentaron™. Fadrozole is marketed under the trade name Afema™. Anastrozole is marketed under the trade name Arimidex™. Letrozole is marketed under the trade names Femara™ or Femar™. Aminoglutethimide is marketed under the trade name Orimeten™. A combination of the invention comprising a chemotherapeutic agent which is an aromatase inhibitor is particularly useful for the treatment of hormone receptor positive tumors, such as breast tumors.

In some embodiments, one or more other therapeutic agent is an mTOR inhibitor, which inhibits cell proliferation, angiogenesis and glucose uptake. In some embodiments, an mTOR inhibitor is everolimus (Afinitor®, Novartis); temsirolimus (Torisel®, Pfizer); and sirolimus (Rapamune®, Pfizer).

In some embodiments, one or more other therapeutic agent is an aromatase inhibitor. In some embodiments, an aromatase inhibitor is selected from exemestane (Aromasin®, Pfizer); anastazole (Arimidex®, AstraZeneca) and letrozole (Femara®, Novartis).

The term “antiestrogen” as used herein relates to a compound which antagonizes the effect of estrogens at the estrogen receptor level. The term includes, but is not limited to tamoxifen, fulvestrant, raloxifene and raloxifene hydrochloride. Tamoxifen is marketed under the trade name Nolvadex™. Raloxifene hydrochloride is marketed under the trade name Evista™. Fulvestrant can be administered under the trade name Faslodex™. A combination of the invention comprising a chemotherapeutic agent which is an antiestrogen is particularly useful for the treatment of estrogen receptor positive tumors, such as breast tumors.

The term “anti-androgen” as used herein relates to any substance which is capable of inhibiting the biological effects of androgenic hormones and includes, but is not limited to, bicalutamide (Casodex™). The term “gonadorelin agonist” as used herein includes, but is not limited to abarelix, goserelin and goserelin acetate. Goserelin can be administered under the trade name Zoladex™.

The term “topoisomerase I inhibitor” as used herein includes, but is not limited to topotecan, gimatecan, irinotecan, camptothecian and its analogues, 9-nitrocamptothecin and the macromolecular camptothecin conjugate PNU-166148. Irinotecan can be administered, e.g. in the form as it is marketed, e.g. under the trademark Camptosar™. Topotecan is marketed under the trade name Hycamptin™.

The term “topoisomerase II inhibitor” as used herein includes, but is not limited to the anthracyclines such as doxorubicin (including liposomal formulation, such as Caelyx™) daunorubicin, epirubicin, idarubicin and nemorubicin, the anthraquinones mitoxantrone and losoxantrone, and the podophillotoxines etoposide and teniposide. Etoposide is marketed under the trade name Etopophos™. Teniposide is marketed under the trade name VM 26-Bristol Doxorubicin is marketed under the trade name Acriblastin™ or Adriamycin™. Epirubicin is marketed under the trade name Farmorubicin™. Idarubicin is marketed. under the trade name Zavedos™. Mitoxantrone is marketed under the trade name Novantron.

The term “microtubule active agent” relates to microtubule stabilizing, microtubule destabilizing compounds and microtublin polymerization inhibitors including, but not limited to taxanes, such as paclitaxel and docetaxel; vinca alkaloids, such as vinblastine or vinblastine sulfate, vincristine or vincristine sulfate, and vinorelbine; discodermolides; cochicine and epothilones and derivatives thereof. Paclitaxel is marketed under the trade name Taxol™. Docetaxel is marketed under the trade name Taxotere™. Vinblastine sulfate is marketed under the trade name Vinblastin R.P™. Vincristine sulfate is marketed under the trade name Farmistin™.

The term “alkylating agent” as used herein includes, but is not limited to, cyclophosphamide, ifosfamide, melphalan or nitrosourea (BCNU or Gliadel). Cyclophosphamide is marketed under the trade name Cyclostin™. Ifosfamide is marketed under the trade name Holoxan™.

The term “histone deacetylase inhibitors” or “HDAC inhibitors” relates to compounds which inhibit the histone deacetylase and which possess antiproliferative activity. This includes, but is not limited to, suberoylanilide hydroxamic acid (SAHA).

The term “antineoplastic antimetabolite” includes, but is not limited to, 5-fluorouracil or 5-FU, capecitabine, gemcitabine, DNA demethylating compounds, such as 5-azacytidine and decitabine, methotrexate and edatrexate, and folic acid antagonists such as pemetrexed. Capecitabine is marketed under the trade name Xeloda™. Gemcitabine is marketed under the trade name Gemzar™.

The term “platin compound” as used herein includes, but is not limited to, carboplatin, cis-platin, cisplatinum and oxaliplatin. Carboplatin can be administered, e.g., in the form as it is marketed, e.g. under the trademark Carboplat™. Oxaliplatin can be administered, e.g., in the form as it is marketed, e.g. under the trademark Eloxatin™.

The term “Bcl-2 inhibitor” as used herein includes, but is not limited to compounds having inhibitory activity against B-cell lymphoma 2 protein (Bcl-2), including but not limited to ABT-199, ABT-731, ABT-737, apogossypol, Ascenta's pan-Bcl-2 inhibitors, curcumin (and analogs thereof), dual Bcl-2/Bcl-xL inhibitors (Infinity Pharmaceuticals/Novartis Pharmaceuticals), Genasense (G3139), HA14-1 (and analogs thereof; see WO 2008/118802, US 2010/0197686), navitoclax (and analogs thereof, see U.S. Pat. No. 7,390,799), NH-1 (Shenayng Pharmaceutical University), obatoclax (and analogs thereof, see WO 2004/106328, US 2005/0014802), S-001 (Gloria Pharmaceuticals), TW series compounds (Univ. of Michigan), and venetoclax. In some embodiments the Bcl-2 inhibitor is a small molecule therapeutic. In some embodiments the Bcl-2 inhibitor is a peptidomimetic.

The term “compounds targeting/decreasing a protein or lipid kinase activity; or a protein or lipid phosphatase activity; or further anti-angiogenic compounds” as used herein includes, but is not limited to, protein tyrosine kinase and/or serine and/or threonine kinase inhibitors or lipid kinase inhibitors, such as a) compounds targeting, decreasing or inhibiting the activity of the platelet-derived growth factor-receptors (PDGFR), such as compounds which target, decrease or inhibit the activity of PDGFR, especially compounds which inhibit the PDGF receptor, such as an N-phenyl-2-pyrimidine-amine derivative, such as imatinib, SU101, SU6668 and GFB-111; b) compounds targeting, decreasing or inhibiting the activity of the fibroblast growth factor-receptors (FGFR); c) compounds targeting, decreasing or inhibiting the activity of the insulin-like growth factor receptor I (IGF-IR), such as compounds which target, decrease or inhibit the activity of IGF-IR, especially compounds which inhibit the kinase activity of IGF-I receptor, or antibodies that target the extracellular domain of IGF-I receptor or its growth factors; d) compounds targeting, decreasing or inhibiting the activity of the Trk receptor tyrosine kinase family, or ephrin B4 inhibitors; e) compounds targeting, decreasing or inhibiting the activity of the AxI receptor tyrosine kinase family; f) compounds targeting, decreasing or inhibiting the activity of the Ret receptor tyrosine kinase; g) compounds targeting, decreasing or inhibiting the activity of the Kit/SCFR receptor tyrosine kinase, such as imatinib; h) compounds targeting, decreasing or inhibiting the activity of the C-kit receptor tyrosine kinases, which are part of the PDGFR family, such as compounds which target, decrease or inhibit the activity of the c-Kit receptor tyrosine kinase family, especially compounds which inhibit the c-Kit receptor, such as imatinib; i) compounds targeting, decreasing or inhibiting the activity of members of the c-Abl family, their gene-fusion products (e.g. BCR-Abl kinase) and mutants, such as compounds which target decrease or inhibit the activity of c-Abl family members and their gene fusion products, such as an N-phenyl-2-pyrimidine-amine derivative, such as imatinib or nilotinib (AMN107); PD180970; AG957; NSC 680410; PD173955 from ParkeDavis; or dasatinib (BMS-354825); j) compounds targeting, decreasing or inhibiting the activity of members of the protein kinase C (PKC) and Raf family of serine/threonine kinases, members of the MEK, SRC, JAK/pan-JAK, FAK, PDK1, PKB/Akt, Ras/MAPK, PI3K, SYK, TYK2, BTK and TEC family, and/or members of the cyclin-dependent kinase family (CDK) including staurosporine derivatives, such as midostaurin; examples of further compounds include UCN-01, safingol, BAY 43-9006, Bryostatin 1, Perifosine; llmofosine; RO 318220 and RO 320432; GO 6976; lsis 3521; LY333531/LY379196; isochinoline compounds; FTIs; PD184352 or QAN697 (a P13K inhibitor) or AT7519 (CDK inhibitor); k) compounds targeting, decreasing or inhibiting the activity of protein-tyrosine kinase inhibitors, such as compounds which target, decrease or inhibit the activity of protein-tyrosine kinase inhibitors include imatinib mesylate (Gleevec™) or tyrphostin such as Tyrphostin A23/RG-50810; AG 99; Tyrphostin AG 213; Tyrphostin AG 1748; Tyrphostin AG 490; Tyrphostin B44; Tyrphostin B44 (+) enantiomer; Tyrphostin AG 555; AG 494; Tyrphostin AG 556, AG957 and adaphostin (4-{[(2,5-dihydroxyphenyl)methyl]amino}-benzoic acid adamantyl ester; NSC 680410, adaphostin); 1) compounds targeting, decreasing or inhibiting the activity of the epidermal growth factor family of receptor tyrosine kinases (EGFR₁ ErbB2, ErbB3, ErbB4 as homo- or heterodimers) and their mutants, such as compounds which target, decrease or inhibit the activity of the epidermal growth factor receptor family are especially compounds, proteins or antibodies which inhibit members of the EGF receptor tyrosine kinase family, such as EGF receptor, ErbB2, ErbB3 and ErbB4 or bind to EGF or EGF related ligands, CP 358774, ZD 1839, ZM 105180; trastuzumab (Herceptin™), cetuximab (Erbitux™), Iressa, Tarceva, OSI-774, C1-1033, EKB-569, GW-2016, E1.1, E2.4, E2.5, E6.2, E6.4, E2.11, E6.3 or E7.6.3, and 7H-pyrrolo-[2,3-d]pyrimidine derivatives; m) compounds targeting, decreasing or inhibiting the activity of the c-Met receptor, such as compounds which target, decrease or inhibit the activity of c-Met, especially compounds which inhibit the kinase activity of c-Met receptor, or antibodies that target the extracellular domain of c-Met or bind to HGF, n) compounds targeting, decreasing or inhibiting the kinase activity of one or more JAK family members (JAK1/JAK2/JAK3/TYK2 and/or pan-JAK), including but not limited to PRT-062070, SB-1578, baricitinib, pacritinib, momelotinib, VX-509, AZD-1480, TG-101348, tofacitinib, and ruxolitinib; o) compounds targeting, decreasing or inhibiting the kinase activity of PI3 kinase (PI3K) including but not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib; and; and q) compounds targeting, decreasing or inhibiting the signaling effects of hedgehog protein (Hh) or smoothened receptor (SMO) pathways, including but not limited to cyclopamine, vismodegib, itraconazole, erismodegib, and IPI-926 (saridegib).

Compounds which target, decrease or inhibit the activity of a protein or lipid phosphatase are e.g. inhibitors of phosphatase 1, phosphatase 2A, or CDC25, such as okadaic acid or a derivative thereof.

In some embodiments, one or more other therapeutic agent is a growth factor antagonist, such as an antagonist of platelet-derived growth factor (PDGF), or epidermal growth factor (EGF) or its receptor (EGFR). Approved PDGF antagonists which may be used in the present invention include olaratumab (Lartruvo®; Eli Lilly). Approved EGFR antagonists which may be used in the present invention include cetuximab (Erbitux®, Eli Lilly); necitumumab (Portrazza®, Eli Lilly), panitumumab (Vectibix®, Amgen); and osimertinib (targeting activated EGFR, Tagrisso®, AstraZeneca).

The term “PI3K inhibitor” as used herein includes, but is not limited to compounds having inhibitory activity against one or more enzymes in the phosphatidylinositol-3-kinase family, including, but not limited to PI3Kα, PI3Kγ, PI3Kδ, PI3Kβ, PI3K-C2α, PI3K-C2β, PI3K-C2γ, Vps34, p110-α, p110-β, p110-γ, p110-δ, p85-α, p85-β, p55-γ, p150, p101, and p87. Examples of PI3K inhibitors useful in this invention include but are not limited to ATU-027, SF-1126, DS-7423, PBI-05204, GSK-2126458, ZSTK-474, buparlisib, pictrelisib, PF-4691502, BYL-719, dactolisib, XL-147, XL-765, and idelalisib.

The term “BTK inhibitor” as used herein includes, but is not limited to compounds having inhibitory activity against Bruton's Tyrosine Kinase (BTK), including, but not limited to AVL-292 and ibrutinib.

The term “SYK inhibitor” as used herein includes, but is not limited to compounds having inhibitory activity against spleen tyrosine kinase (SYK), including but not limited to PRT-062070, R-343, R-333, Excellair, PRT-062607, and fostamatinib

Further examples of BTK inhibitory compounds, and conditions treatable by such compounds in combination with compounds of this invention can be found in WO 2008/039218, US 2008/0108636 and WO 2011/090760, US 2010/0249092, the entirety of each of which is herein incorporated by reference.

Further examples of SYK inhibitory compounds, and conditions treatable by such compounds in combination with compounds of this invention can be found in WO 2003/063794, US 2004/0029902, WO 2005/007623, US 2005/0075306, and WO 2006/078846, US 2006/0211657, the entirety of each of which is herein incorporated by reference.

Further examples of PI3K inhibitory compounds, and conditions treatable by such compounds in combination with compounds of this invention can be found in WO 2004/019973, US 2004/0106569, WO 2004/089925, US 2004/0242631, US 8,138,347, WO 2002/088112, US 2004/0116421, WO 2007/084786, US 2010/0249126, WO 2007/129161, US 2008/0076768, WO 2006/122806, US 2008/0194579, WO 2005/113554, US 2008/0275067, and WO 2007/044729, US 2010/0087440, the entirety of each of which is herein incorporated by reference.

Further examples of JAK inhibitory compounds, and conditions treatable by such compounds in combination with compounds of this invention can be found in WO 2009/114512, US 2009/0233903, WO 2008/109943, US 2010/0197671, WO 2007/053452, US 2007/0191405, WO 2001/0142246, US 2001/0053782, and WO 2007/070514, US 2007/0135461, the entirety of each of which is herein incorporated by reference.

Further anti-angiogenic compounds include compounds having another mechanism for their activity, e.g. unrelated to protein or lipid kinase inhibition e.g. thalidomide (Thalomid™) and TNP-470.

Examples of proteasome inhibitors useful for use in combination with compounds of the invention include, but are not limited to bortezomib, disulfiram, epigallocatechin-3-gallate (EGCG), salinosporamide A, carfilzomib, ONX-0912, CEP-18770, and MLN9708.

Compounds which target, decrease or inhibit the activity of a protein or lipid phosphatase are e.g. inhibitors of phosphatase 1, phosphatase 2A, or CDC25, such as okadaic acid or a derivative thereof.

Compounds which induce cell differentiation processes include, but are not limited to, retinoic acid, α- γ- or δ- tocopherol or α- γ- or δ-tocotrienol.

The term cyclooxygenase inhibitor as used herein includes, but is not limited to, Cox-2 inhibitors, 5-alkyl substituted 2-arylaminophenylacetic acid and derivatives, such as celecoxib (Celebrex™), rofecoxib (Vioxx™), etoricoxib, valdecoxib or a 5-alkyl-2-arylaminophenylacetic acid, such as 5-methyl-2-(2′-chloro-6′-fluoroanilino)phenyl acetic acid, lumiracoxib.

The term “bisphosphonates” as used herein includes, but is not limited to, etridonic, clodronic, tiludronic, pamidronic, alendronic, ibandronic, risedronic and zoledronic acid. Etridonic acid is marketed under the trade name Didronel™. Clodronic acid is marketed under the trade name Bonefos™. Tiludronic acid is marketed under the trade name Skelid™. Pamidronic acid is marketed under the trade name Aredia™. Alendronic acid is marketed under the trade name Fosamax™. Ibandronic acid is marketed under the trade name Bondranat™. Risedronic acid is marketed under the trade name Actonel™. Zoledronic acid is marketed under the trade name Zometa™. The term “mTOR inhibitors” relates to compounds which inhibit the mammalian target of rapamycin (mTOR) and which possess antiproliferative activity such as sirolimus (Rapamune®), everolimus (Certican™), CCI-779 and ABT578.

The term “heparanase inhibitor” as used herein refers to compounds which target, decrease or inhibit heparin sulfate degradation. The term includes, but is not limited to, PI-88. The term “biological response modifier” as used herein refers to a lymphokine or interferons.

The term “inhibitor of Ras oncogenic isoforms”, such as H-Ras, K-Ras, or N-Ras, as used herein refers to compounds which target, decrease or inhibit the oncogenic activity of Ras; for example, a “farnesyl transferase inhibitor” such as L-744832, DK8G557 or R115777 (Zarnestra™). The term “telomerase inhibitor” as used herein refers to compounds which target, decrease or inhibit the activity of telomerase. Compounds which target, decrease or inhibit the activity of telomerase are especially compounds which inhibit the telomerase receptor, such as telomestatin.

The term “methionine aminopeptidase inhibitor” as used herein refers to compounds which target, decrease or inhibit the activity of methionine aminopeptidase. Compounds which target, decrease or inhibit the activity of methionine aminopeptidase include, but are not limited to, bengamide or a derivative thereof.

The term “proteasome inhibitor” as used herein refers to compounds which target, decrease or inhibit the activity of the proteasome. Compounds which target, decrease or inhibit the activity of the proteasome include, but are not limited to, Bortezomib (Velcade™),); carfilzomib (Kyprolis®, Amgen); and ixazomib (Ninlaro®, Takeda), and MLN 341.

The term “matrix metalloproteinase inhibitor” or (“MMP” inhibitor) as used herein includes, but is not limited to, collagen peptidomimetic and nonpeptidomimetic inhibitors, tetracycline derivatives, e.g. hydroxamate peptidomimetic inhibitor batimastat and its orally bioavailable analogue marimastat (BB-2516), prinomastat (AG3340), metastat (NSC 683551) BMS-279251, BAY 12-9566, TAA211, MMI270B or AAJ996.

The term “compounds used in the treatment of hematologic malignancies” as used herein includes, but is not limited to, FMS-like tyrosine kinase inhibitors, which are compounds targeting, decreasing or inhibiting the activity of FMS-like tyrosine kinase receptors (Flt-3R); interferon, 1-β-D-arabinofuransylcytosine (ara-c) and bisulfan; and ALK inhibitors, which are compounds which target, decrease or inhibit anaplastic lymphoma kinase.

Compounds which target, decrease or inhibit the activity of FMS-like tyrosine kinase receptors (Flt-3R) are especially compounds, proteins or antibodies which inhibit members of the Flt-3R receptor kinase family, such as PKC412, midostaurin, a staurosporine derivative, SU11248 and MLN518.

The term “HSP90 inhibitors” as used herein includes, but is not limited to, compounds targeting, decreasing or inhibiting the intrinsic ATPase activity of HSP90; degrading, targeting, decreasing or inhibiting the HSP90 client proteins via the ubiquitin proteosome pathway. Compounds targeting, decreasing or inhibiting the intrinsic ATPase activity of HSP90 are especially compounds, proteins or antibodies which inhibit the ATPase activity of HSP90, such as 17-allylamino,17-demethoxygeldanamycin (17AAG), a geldanamycin derivative; other geldanamycin related compounds; radicicol and HDAC inhibitors.

The term “antiproliferative antibodies” as used herein includes, but is not limited to, trastuzumab (Herceptin™), Trastuzumab-DM1, erbitux, bevacizumab (Avastin™), rituximab (Rituxan) PRO64553 (anti-CD40) and 2C4 Antibody. By antibodies is meant intact monoclonal antibodies, polyclonal antibodies, multispecific antibodies formed from at least 2 intact antibodies, and antibodies fragments so long as they exhibit the desired biological activity.

For the treatment of acute myeloid leukemia (AML), compounds of the current invention can be used in combination with standard leukemia therapies, especially in combination with therapies used for the treatment of AML. In particular, compounds of the current invention can be administered in combination with, for example, farnesyl transferase inhibitors and/or other drugs useful for the treatment of AML, such as Daunorubicin, Adriamycin, Ara-C, VP-16, Teniposide, Mitoxantrone, Idarubicin, Carboplatinum and PKC412.

Other anti-leukemic compounds include, for example, Ara-C, a pyrimidine analog, which is the 2′-alpha-hydroxy ribose (arabinoside) derivative of deoxycytidine. Also included is the purine analog of hypoxanthine, 6-mercaptopurine (6-MP) and fludarabine phosphate. Compounds which target, decrease or inhibit activity of histone deacetylase (HDAC) inhibitors such as sodium butyrate and suberoylanilide hydroxamic acid (SAHA) inhibit the activity of the enzymes known as histone deacetylases. Specific HDAC inhibitors include MS275, SAHA, FK228 (formerly FR901228), Trichostatin A and compounds disclosed in U.S. Pat. No. 6,552,065 including, but not limited to, N-hydroxy-3-[4-[[[2-(2-methyl-1H-indol-3-yl)-ethyl]-amino]methyl]phenyl]-2E-2-propenamide, or a pharmaceutically acceptable salt thereof and N-hydroxy-3-[4-[(2-hydroxyethyl){2-(1H-indol-3-yl)ethyl]-amino]methyl]phenyl]-2E-2-propenamide, or a pharmaceutically acceptable salt thereof, especially the lactate salt. Somatostatin receptor antagonists as used herein refer to compounds which target, treat or inhibit the somatostatin receptor such as octreotide, and SOM230. Tumor cell damaging approaches refer to approaches such as ionizing radiation. The term “ionizing radiation” referred to above and hereinafter means ionizing radiation that occurs as either electromagnetic rays (such as X-rays and gamma rays) or particles (such as alpha and beta particles). Ionizing radiation is provided in, but not limited to, radiation therapy and is known in the art. See Hellman, Principles of Radiation Therapy, Cancer, in Principles and Practice of Oncology, Devita et al., Eds., 4^{th} Edition, Vol. 1 , pp. 248-275 (1993).

Also included are EDG binders and ribonucleotide reductase inhibitors. The term “EDG binders” as used herein refers to a class of immunosuppressants that modulates lymphocyte recirculation, such as FTY720. The term “ribonucleotide reductase inhibitors” refers to pyrimidine or purine nucleoside analogs including, but not limited to, fludarabine and/or cytosine arabinoside (ara-C), 6-thioguanine, 5-fluorouracil, cladribine, 6-mercaptopurine (especially in combination with ara-C against ALL) and/or pentostatin. Ribonucleotide reductase inhibitors are especially hydroxyurea or 2-hydroxy-1H-isoindole-1,3-dione derivatives.

Also included are in particular those compounds, proteins or monoclonal antibodies of VEGF such as 1-(4-chloroanilino)-4-(4-pyridylmethyl)phthalazine or a pharmaceutically acceptable salt thereof, 1-(4-chloroanilino)-4-(4-pyridylmethyl)phthalazine succinate; Angiostatin™; Endostatin™; anthranilic acid amides; ZD4190; ZD6474; SU5416; SU6668; bevacizumab; or anti-VEGF antibodies or anti-VEGF receptor antibodies, such as rhuMAb and RHUFab, VEGF aptamer such as Macugon; FLT-4 inhibitors, FLT-3 inhibitors, VEGFR-2 IgGI antibody, Angiozyme (RPI 4610) and Bevacizumab (Avastin™).

Photodynamic therapy as used herein refers to therapy which uses certain chemicals known as photosensitizing compounds to treat or prevent cancers. Examples of photodynamic therapy include treatment with compounds, such as Visudyne™ and porfimer sodium.

Angiostatic steroids as used herein refers to compounds which block or inhibit angiogenesis, such as, e.g., anecortave, triamcinolone, hydrocortisone, 11-α-epihydrocotisol, cortexolone, 17α-hydroxyprogesterone, corticosterone, desoxycorticosterone, testosterone, estrone and dexamethasone.

Implants containing corticosteroids refers to compounds, such as fluocinolone and dexamethasone.

Other chemotherapeutic compounds include, but are not limited to, plant alkaloids, hormonal compounds and antagonists; biological response modifiers, preferably lymphokines or interferons; antisense oligonucleotides or oligonucleotide derivatives; shRNA or siRNA; or miscellaneous compounds or compounds with other or unknown mechanism of action.

The compounds of the invention are also useful as co-therapeutic compounds for use in combination with other drug substances such as anti-inflammatory, bronchodilatory or antihistamine drug substances, particularly in the treatment of obstructive or inflammatory airways diseases such as those mentioned hereinbefore, for example as potentiators of therapeutic activity of such drugs or as a means of reducing required dosaging or potential side effects of such drugs. A compound of the invention may be mixed with the other drug substance in a fixed pharmaceutical composition or it may be administered separately, before, simultaneously with or after the other drug substance. Accordingly the invention includes a combination of a compound of the invention as hereinbefore described with an anti-inflammatory, bronchodilatory, antihistamine or anti-tussive drug substance, said compound of the invention and said drug substance being in the same or different pharmaceutical composition.

Suitable anti-inflammatory drugs include steroids, in particular glucocorticosteroids such as budesonide, beclamethasone dipropionate, fluticasone propionate, ciclesonide or mometasone furoate; non-steroidal glucocorticoid receptor agonists; LTB4 antagonists such LY293111, CGS025019C, CP-195543, SC-53228, BIIL 284, ONO 4057, SB 209247; LTD4 antagonists such as montelukast and zafirlukast; PDE4 inhibitors such cilomilast (Ariflo® GlaxoSmithKline), Roflumilast (Byk Gulden), V-11294A (Napp), BAY19-8004 (Bayer), SCH-351591 (Schering-Plough), Arofylline (Almirall Prodesfarma), PD189659/PD168787 (Parke-Davis), AWD-12-281 (Asta Medica), CDC-801 (Celgene), SeICID™ CC-10004 (Celgene), VM554/UM565 (Vernalis), T-440 (Tanabe), KW-4490 (Kyowa Hakko Kogyo); A2a agonists; A2b antagonists; and beta-2 adrenoceptor agonists such as albuterol (salbutamol), metaproterenol, terbutaline, salmeterol fenoterol, procaterol, and especially, formoterol and pharmaceutically acceptable salts thereof. Suitable bronchodilatory drugs include anticholinergic or antimuscarinic compounds, in particular ipratropium bromide, oxitropium bromide, tiotropium salts and CHF 4226 (Chiesi), and glycopyrrolate.

Suitable antihistamine drug substances include cetirizine hydrochloride, acetaminophen, clemastine fumarate, promethazine, loratidine, desloratidine, diphenhydramine and fexofenadine hydrochloride, activastine, astemizole, azelastine, ebastine, epinastine, mizolastine and tefenadine.

Other useful combinations of compounds of the invention with anti-inflammatory drugs are those with antagonists of chemokine receptors, e.g. CCR-1, CCR-2, CCR-3, CCR-4, CCR-5, CCR-6, CCR-7, CCR-8, CCR-9 and CCR10, CXCR1, CXCR2, CXCR3, CXCR4, CXCRS, particularly CCR-5 antagonists such as Schering-Plough antagonists SC-351125, SCH-55700 and SCH-D, and Takeda antagonists such as N-[[4-[[[6,7-dihydro-2-(4-methylphenyl)-5H-benzo-cyclohepten-8-yl]carbonyl]amino]phenyl]-methyl]tetrahydro-N,N-dimethyl-2H-pyran-4-aminium chloride (TAK-770).

The structure of the active compounds identified by code numbers, generic or trade names may be taken from the actual edition of the standard compendium “The Merck Index” or from databases, e.g. Patents International (e.g. IMS World Publications).

A compound of the current invention may also be used in combination with known therapeutic processes, for example, the administration of hormones or radiation. In certain embodiments, a provided compound is used as a radiosensitizer, especially for the treatment of tumors which exhibit poor sensitivity to radiotherapy.

A compound of the current invention can be administered alone or in combination with one or more other therapeutic compounds, possible combination therapy taking the form of fixed combinations or the administration of a compound of the invention and one or more other therapeutic compounds being staggered or given independently of one another, or the combined administration of fixed combinations and one or more other therapeutic compounds. A compound of the current invention can besides or in addition be administered especially for tumor therapy in combination with chemotherapy, radiotherapy, immunotherapy, phototherapy, surgical intervention, or a combination of these. Long-term therapy is equally possible as is adjuvant therapy in the context of other treatment strategies, as described above. Other possible treatments are therapy to maintain the patient's status after tumor regression, or even chemopreventive therapy, for example in patients at risk.

Those additional agents may be administered separately from an inventive compound-containing composition, as part of a multiple dosage regimen. Alternatively, those agents may be part of a single dosage form, mixed together with a compound of this invention in a single composition. If administered as part of a multiple dosage regime, the two active agents may be submitted simultaneously, sequentially or within a period of time from one another normally within five hours from one another.

As used herein, the term “combination,” “combined,” and related terms refers to the simultaneous or sequential administration of therapeutic agents in accordance with this invention. For example, a compound of the present invention may be administered with another therapeutic agent simultaneously or sequentially in separate unit dosage forms or together in a single unit dosage form. Accordingly, the present invention provides a single unit dosage form comprising a compound of the current invention, an additional therapeutic agent, and a pharmaceutically acceptable carrier, adjuvant, or vehicle.

The amount of both an inventive compound and additional therapeutic agent (in those compositions which comprise an additional therapeutic agent as described above) that may be combined with the carrier materials to produce a single dosage form will vary depending upon the host treated and the particular mode of administration. Preferably, compositions of this invention should be formulated so that a dosage of between 0.01-100 mg/kg body weight/day of an inventive compound can be administered.

In those compositions which comprise an additional therapeutic agent, that additional therapeutic agent and the compound of this invention may act synergistically. Therefore, the amount of additional therapeutic agent in such compositions will be less than that required in a monotherapy utilizing only that therapeutic agent. In such compositions a dosage of between 0.01-1,000 μg/kg body weight/day of the additional therapeutic agent can be administered.

The amount of one or more other therapeutic agent present in the compositions of this invention may be no more than the amount that would normally be administered in a composition comprising that therapeutic agent as the only active agent. Preferably the amount of one or more other therapeutic agent in the presently disclosed compositions will range from about 50% to 100% of the amount normally present in a composition comprising that agent as the only therapeutically active agent. In some embodiments, one or more other therapeutic agent is administered at a dosage of about 50%, about 55%, about 60%, about 65%, about 70%, about 75%, about 80%, about 85%, about 90%, or about 95% of the amount normally administered for that agent. As used herein, the phrase “normally administered” means the amount an FDA approved therapeutic agent is approvided for dosing per the FDA label insert.

The compounds of this invention, or pharmaceutical compositions thereof, may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

Exemplary Immuno-Oncology Agents

In some embodiments, one or more other therapeutic agent is an immuno-oncology agent. As used herein, the term “an immuno-oncology agent” refers to an agent which is effective to enhance, stimulate, and/or up-regulate immune responses in a subject. In some embodiments, the administration of an immuno-oncology agent with a compound of the invention has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a compound of the invention and an immuno-oncology agent can stimulate T cell responses. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX4OL, GITR, GITRL, CD70, CD27, CD40, DR3 and CD28H.

In some embodiments, an immuno-oncology agent is an antagonist of inhibitory receptors on NK cells or an agonists of activating receptors on NK cells. In some embodiments, an immuno-oncology agent is an antagonists of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO 2011/070024, US 2011/0165156, WO 2011/0107553, US 2012/0329997, WO 2011/131407, US 2013/0005949, WO 2013/087699, US 2014/0336363, WO 2013/119716, WO 2013/132044, US 2014/0079706) or FPA-008 (WO 2011/140249, US 2011/0274683; WO 2013/169264; WO 2014/036357, US 2014/0079699).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO 2010/077634, US 2010/0203056), durvalumab (MEDI4736), BMS-936559 (WO 2007/005874, US 2009/0055944), and MSB0010718C (WO 2013/079174, US 2014/0341917).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO 2010/019570, US 2010/0150892, WO 2014/008218, US 2014/0093511), or IMP-731 or IMP-321 (WO 2008/132601, US 2010/0233183, WO 2009/044273, US 2011/0008331).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO 2006/105021, US 2007/0098719, WO 2009/009116, US 2009/0136494), or MK-4166 (WO 2011/028683, US 2012/0189639).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS :F001287 (Bristol-Myers Squibb); Phy906/KD108 (Phytoceutica); an enzyme that breaks down kynurenine (Kynase, Kyn Therapeutics); and NLG-919 (WO 2009/073620, US 2011/0053941, WO 2009/132238, US 2011/0136796, WO 2011/056652, US 2012/0277217, WO 2012/142237, US 2014/0066625).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO 2006/029879, U.S. Pat. No. 7,501,496).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO 2011/109400, US 2013/0149236).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (Opdivo®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (Pomalyst®, Celgene); lenalidomide (Revlimid®, Celgene); ingenol mebutate (Picato®, LEO Pharma).

In some embodiments, an immuno-oncology agent is a cancer vaccine. In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-)deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-C SF, in melanoma (NCT03003676); and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TGO2 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8⁺ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682, the entirety of each of which is herein incorporated by reference, which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials.gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+ (Th17) and CD8+ (Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that may be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetlL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams E T. A L., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams E T. A L. In some embodiments, an immuno-oncology agent is a small molecule targeting an immuno-oncoloby target selected from those listed in Table 2 of Jerry L. Adams E T. A L. In some embodiments, an immuno-oncology agent is a small molecule agent selected from those listed in Table 2 of Jerry L. Adams E T. A L.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BiTE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BiTE®) antibody construct. In some embodiments, a bispecific T cell engager (BiTE®) antibody construct is a CD19/CD3 bispecific antibody construct. In some embodiments, a bispecific T cell engager (BiTE®) antibody construct is an EGFR/CD3 bispecific antibody construct. In some embodiments, a bispecific T cell engager (BiTE®) antibody construct activates T cells. In some embodiments, a bispecific T cell engager (BiTE®) antibody construct activates T cells, which release cytokines inducing upregulation of intercellular adhesion molecule 1 (ICAM-1) and FAS on bystander cells. In some embodiments, a bispecific T cell engager (BiTE®) antibody construct activates T cells which result in induced bystander cell lysis. In some embodiments, the bystander cells are in solid tumors. In some embodiments, the bystander cells being lysed are in proximity to the BiTE®-acticvated T cells. In some embodiment, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex-vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

Exemplary Immune Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In one aspect, the checkpoint inhibitor is a biologic therapeutic or a small molecule. In another aspect, the checkpoint inhibitor is a monoclonal antibody, a humanized antibody, a fully human antibody, a fusion protein or a combination thereof In a further aspect, the checkpoint inhibitor inhibits a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In an additional aspect, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof In an aspect, the checkpoint inhibitor is an immunostimulatory agent, a T cell growth factor, an interleukin, an antibody, a vaccine or a combination thereof In a further aspect, the interleukin is IL-7 or IL-15. In a specific aspect, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors may include small molecule inhibitors or may include antibodies, or antigen binding fragments thereof, that bind to and block or inhibit immune checkpoint receptors or antibodies that bind to and block or inhibit immune checkpoint receptor ligands. Illustrative checkpoint molecules that may be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, γδ, and memory CD8⁺ (αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (Opdivo®), ipilimumab (Yervoy®), and pembrolizumab (Keytruda®). In some embodiments, the checkpoint inhibitor is selected from nivolumab (anti-PD-1 antibody, Opdivo®, Bristol-Myers Squibb); pembrolizumab (anti-PD-1 antibody, Keytruda®, Merck); ipilimumab (anti-CTLA-4 antibody, Yervoy®, Bristol-Myers Squibb); durvalumab (anti-PD-L1 antibody, Imfinzi®, AstraZeneca); and atezolizumab (anti-PD-L1 antibody, Tecentriq®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (Keytruda®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (Bavencio®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IMP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that may be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that may be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981).

Checkpoint inhibitors that may be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that may be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that may be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that may be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that may be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that may be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that may be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that may be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that may be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

Abbreviations

Ac: acetyl

AcOH: acetic acid

ACN: acetonitrile

Ad: adamantly

AIBN: 2,2′-azo bisisobutyronitrile

Anhyd: anhydrous

Aq: aqueous

B₂Pin₂: bis (pinacolato)diboron-4,4,4′,4′,5,5,5′,5′-octamethyl-2,2′-bi(1,3,2-dioxaborolane)

BINAP: 2,2′-bis(diphenylphosphino)-1,1′-binaphthyl

BH₃: Borane

Bn: benzyl

Boc: tert-butoxycarbonyl

Boc₂O: di-tert-butyl dicarbonate

BPO: benzoyl peroxide

ⁿBuOH: n-butanol

CDI: carbonyldiimidazole

COD: cyclooctadiene

d: days

DABCO: 1,4-diazobicyclo[2.2.2]octane

DAST: diethylaminosulfur trifluoride

dba: dibenzylideneacetone

DBU: 1,8-diazobicyclo[5.4.0]undec-7-ene

DCE: 1,2-dichloroethane

DCM: dichloromethane

DEA: diethylamine

DHP: dihydropyran

DIBAL-H: diisobutylaluminum hydride

DIPA: diisopropylamine

DIPEA or DIEA: N,N-diisopropylethylamine

DMA: N,N-dimethylacetamide

DME: 1,2-dimethoxyethane

DMAP: 4-dimethylaminopyridine

DMF: N,N-dimethylformamide

DMP: Dess-Martin periodinane

DMSO-dimethyl sulfoxide

DPPA: diphenylphosphoryl azide

dppf: 1,1′-bis(diphenylphosphino)ferrocene

EDC or EDCI: 1-(3-dimethylaminopropyl)-3-ethylcarbodiimide hydrochloride

ee: enantiomeric excess

ESI: electrospray ionization

EA: ethyl acetate

EtOAc: ethyl acetate

EtOH: ethanol

FA: formic acid

h or hrs: hours

HATU: N,N,N′,N′-tetramethyl-O-(7-azabenzotriazol-1-yl)uronium hexafluorophosphate

HCl: hydrochloric acid

HPLC: high performance liquid chromatography

HOAc: acetic acid

IBX: 2-iodoxybenzoic acid

IPA: isopropyl alcohol

KHMDS: potassium hexamethyldisilazide

K₂CO₃: potassium carbonate

LAH: lithium aluminum hydride

LDA: lithium diisopropylamide

m-CPBA: meta-chloroperbenzoic acid

M: molar

MeCN: acetonitrile

MeOH: methanol

Me₂S: dimethyl sulfide

MeONa: sodium methylate

MeI: iodomethane

min: minutes

mL: milliliters

mM: millimolar

mmol: millimoles

MPa: mega pascal

MOMCl: methyl chloromethyl ether

MsCl: methanesulfonyl chloride

MTBE: methyl tert-butyl ether

nBuLi: n-butyllithium

NaNO₂: sodium nitrite

NaOH: sodium hydroxide

Na₂SO₄: sodium sulfate

NBS: N-bromosuccinimide

NCS: N-chlorosuccinimide

NFSI: N-Fluorobenzenesulfonimide

NMO: N-methylmorpholine N-oxide

NMP: N-methylpyrrolidine

NMR: Nuclear Magnetic Resonance

° C.: degrees Celsius

Pd/C: Palladium on Carbon

Pd(OAc)₂: Palladium Acetate

PBS: phosphate buffered saline

PE: petroleum ether

POCl₃: phosphorus oxychloride

PPh₃: triphenylphosphine

PyBOP: (Benzotriazol-1-yloxy)tripyrrolidinophosphonium hexafluorophosphate

Rel: relative

R.T. or rt: room temperature

sat: saturated

SEMCl: chloromethyl-2-trimethylsilylethyl ether

SFC: supercritical fluid chromatography

SOCl₂: sulfur dichloride

tBuOK: potassium tert-butoxide

TBAB: tetrabutylammonium bromide

TBAI: tetrabutylammonium iodide

TEA: triethylamine

Tf: trifluoromethanesulfonate

TfAA, TFMSA or Tf₂O: trifluoromethanesulfonic anhydride

TFA: trifluoracetic acid

TIPS: triisopropylsilyl

THF: tetrahydrofuran

THP: tetrahydropyran

TLC: thin layer chromatography

TMEDA: tetramethylethylenediamine

pTSA: para-toluenesulfonic acid

wt: weight

Xantphos: 4,5-bis(diphenylphosphino)-9,9-dimethylxanthene

### General Synthetic Methods

The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg(=20-133 mbar). The structure of final products, intermediates and starting materials is confirmed by standard analytical methods, e.g., microanalysis and spectroscopic characteristics, e.g., MS, IR, NMR. Abbreviations used are those conventional in the art.

All starting materials, building blocks, reagents, acids, bases, dehydrating agents, solvents, and catalysts utilized to synthesis the compounds of the present invention are either commercially available or can be produced by organic synthesis methods known to one of ordinary skill in the art (Houben-Weyl 4th Ed. 1952, Methods of Organic Synthesis, Thieme, Volume 21). Further, the compounds of the present invention can be produced by organic synthesis methods known to one of ordinary skill in the art as shown in the following examples.

All reactions are carried out under nitrogen or argon unless otherwise stated.

Proton NMR (¹H NMR) is conducted in deuterated solvent. In certain compounds disclosed herein, one or more ¹H shifts overlap with residual proteo solvent signals; these signals have not been reported in the experimental provided hereinafter.

As depicted in the Examples below, in certain exemplary embodiments, compounds are prepared according to the following general procedures. It will be appreciated that, although the general methods depict the synthesis of certain compounds of the present invention, the following general methods, and other methods known to one of ordinary skill in the art, can be applied to all compounds and subclasses and species of each of these compounds, as described herein.

### KEY INTERMEDIATE PREPARATION

The following intermediates, A, B, C, D, E, F, G, H, and I, were prepared according to the methods below and further utilized in the syntheses of compounds depicted in Examples 1 through 15.

### INTERMEDIATE A: 14-((2-(2,6-dioxopiperidin-3-yl)-1,3-dioxoisoindolin-4-yl)amino)-3,6,9,12-tetraoxatetradecanal

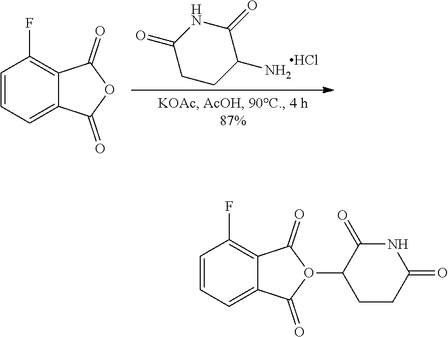

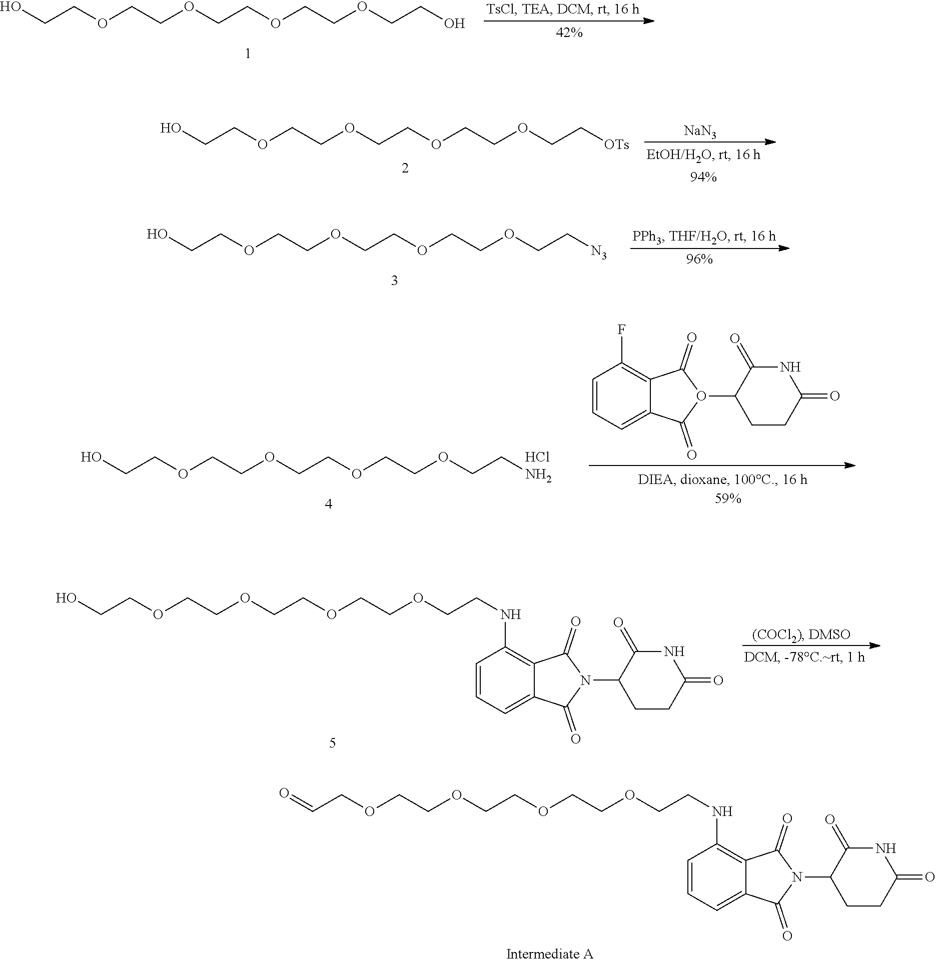

### Step 1: 2-(2,6-Dioxopiperidin-3-yl)-4-fluoro-2,3-dihydro-1H-isoindole-1,3-dione

To a stirred mixture of 4-fluoro-1,3-dihydro-2-benzofuran-1,3-dione (25 g, 150.5 mmol) and 3-aminopiperidine-2,6-dione (21.2 g, 165.5 mmol) in AcOH (400 mL) was added KOAc (49.7 g, 506.4 mmol) at room temperature under nitrogen atmosphere. The resulting mixture was stirred for 4 hours at 90° C. under nitrogen atmosphere. Upon completion, the resulting mixture was cooled to room temperature and poured into ice/water (1.5 L). The precipitated solids were collected by filtration, washed with water (3×50 mL) and dried in the air to afford 2-(2,6-dioxopiperidin-3-yl)-4-fluoro-2,3-dihydro-1H-isoindole-1,3-dione (36 g, 87%) as a grey solid. ¹H NMR (400 MHz, DMSO-d₆) δ 11.16 (br, 1H), 7.95 (td, J=7.9, 4.4 Hz, 1H), 7.79 (d, J=7.3 Hz, 1H), 7.73 (t, J=8.9 Hz, 1H), 5.17 (dd, J=12.8, 5.4 Hz, 1H), 2.91 (ddd, J=17.2, 13.8, 5.4 Hz, 1H), 2.66-2.46 (m, 2H), 2.08 (ddq, J=10.7, 5.6, 3.2, 2.6 Hz, 1H). LC/MS (ESI, m/z): [(M+18)]⁺=294.10

### Step 2: 14-[(4-Methylbenzenesulfonyl)oxy]-3,6,9,12-tetraoxatetradecan-1-ol

To a stirred solution of 3,6,9,12-tetraoxatetradecane-1,14-diol (70 g, 293.8 mmol) in DCM (500 mL) was added TEA (29.7 g, 293.8 mmol) followed by the addition of a solution of 4-methylbenzene-1-sulfonyl chloride (56.0 g, 293.8 mmol) in DCM (500 mL) over 30 min at room temperature under nitrogen atmosphere. The resulting solution was stirred for an additional 16 h at room temperature. Upon completion, the resulting solution was concentrated under reduced pressure and the residue was purified by silica gel column chromatography, eluted with EtOAc to afford 14-[(4-methylbenzenesulfonyl)oxy]-3,6,9,12-tetraoxatetradecan-1-ol (48 g, 42%) as a light yellow oil.

¹H NMR (400 MHz, CDCl₃) δ 7.81 (d, J=8.0 Hz, 2H), 7.35 (d, J=8.0 Hz, 2H), 4.17 (t, J=4.9 Hz, 2H), 3.80-3.56 (m, 18H), 2.50 (d, J=4.4 Hz, 1H), 2.46 (s, 3H) LC/MS (ESI, m/z): [(M+1)]⁺=393.00

### Step 3: 14-Azido-3,6,9,12-tetraoxatetradecan-1-ol

A solution of 14-[(4-methylbenzenesulfonyl)oxy]-3,6,9,12-tetraoxatetradecan-1-ol (44 g, 112.1 mmol) and NaN₃ (29.2 g, 448.5 mmol) in EtOH (300 mL) and H₂O (300 mL) was stirred for 16 h at 80° C. under nitrogen atmosphere. Upon completion, the resulting solution was concentrated under reduced pressure to remove EtOH. The residue was diluted with water (200 mL) and extracted with EtOAc (4×400 mL). The combined organic layers was washed with brine (500 mL) and dried over anhydrous Na₂SO₄. After filtration, the filtrate was concentrated under reduced pressure and the residue was purified by silica gel column chromatography, eluted with 5% methanol in dichloromethane to afford 14-azido-3,6,9,12-tetraoxatetradecan-1-ol (27.7 g, 94%) as a light yellow oil. ¹H NMR (400 MHz, CDCl₃) δ 3.71 (t, J=4.5 Hz, 2H), 3.66 (s, 14H), 3.60 (t, J=4.5 Hz, 2H), 3.38 (t, J=5.1 Hz, 2H), 2.65 (br s, 1H). LC/MS (ESI, m/z): [(M+1)]⁺=264.00

### Step 4: 14-Amino-3,6,9,12-tetraoxatetradecan-1-ol hydrochloride

To a solution of 14-azido-3,6,9,12-tetraoxatetradecan-1-ol (27.7 g, 105.2 mmol) in THF (300 mL) were added H₂O (60 mL) and Ph₃P (41.4 g, 157.8 mmol) (several batches). After stirring for an additional 16 h at room temperature under nitrogen atmosphere, the resulting solution was concentrated under reduced pressure and the residue was taken up with water (50 mL) and acidified to pH=1 with concentrated hydrochloric acid. The aqueous layer was extracted with CH₂Cl₂ (3×30 mL). The aqueous layer was concentrated under reduced pressure to afford 14-amino-3,6,9,12-tetraoxatetradecan-1-ol hydrochloride (27.6 g, 96%) as a light yellow oil. ¹H NMR (400 MHz, CDCl₃) δ 7.89 (br s, 3H), 5.00 (s, 2H), 3.90-3.80 (m, 2H), 3.79-3.72 (m, 2H), 3.71-3.55 (m, 13H), 3.26-3.13 (m, 2H). LC/MS (ESI, m/z): [(M+1)]⁺=238.10

### Step 5: 2-(2,6-Dioxopiperidin-3-yl)-4-(15-hydroxy-4,7,10,13-tetraoxa-1-azapentadecan-1-yl)-2,3-dihydro-1H-isoindole-1,3-dione

To a solution of 14-amino-3,6,9,12-tetraoxatetradecan-1-ol hydrochloride (10 g, 36.5 mmol) in 1,4-dioxane (100 mL) were added 2-(2,6-dioxopiperidin-3-yl)-4-fluoro-2,3-dihydro-1H-isoindole-1,3-dione (12.1 g, 43.8 mmol) and DIEA (7.4 g, 73.1 mmol). The resulting solution was stirred for 16 h at 100° C. under nitrogen atmosphere. The resulting solution was concentrated under reduced pressure and the residue was purified by silica gel column chromatography, eluted with 50% acetonitrile in dichloromethane to afford 2-(2,6-dioxopiperidin-3-yl)-4-(15-hydroxy-4,7,10,13-tetraoxa-1-azapentadecan-1-yl)-2,3-dihydro-1H-isoindole-1,3-dione (10.7 g, 59%) as a light yellow solid. ¹H NMR (400 MHz, CDCl₃) δ 8.48 (br s, 1H), 7.51 (t, J=7.8 Hz, 1H), 7.13 (d, J=7.1 Hz, 1H), 6.94 (d, J=8.5 Hz, 1H), 4.93 (dd, J=12.0, 5.3 Hz, 1H), 3.79-3.60 (m, 18H), 3.49 (t, J=5.4 Hz, 2H), 2.94-2.69 (m, 3H), 2.21-2.08 (m, 1H). LC/MS (ESI, m/z): [(M+1)]⁺=494.35

### Step 6: 14-[[2-(2,6-Dioxopiperidin-3-yl)-1,3-dioxo-2,3-dihydro-1H-isoindol-4-yl]amino]-3,6,9,12-tetraoxatetradecanal

To a stirred solution of oxalic dichloride (0.7 g, 5.47 mmol) in DCM (50 mL) was added DMSO (0.6 g, 7.29 mmol) dropwise at −78° C. under nitrogen atmosphere. The solution was stirred for 30 min at −78° C. Then a solution of 2-(2,6-dioxopiperidin-3-yl)-4-(15-hydroxy-4,7,10,13-tetraoxa-1-azapentadecan-1-yl)-2,3-dihydro-1H-isoindole-1,3-dione (1.8 g, 3.65 mmol) in DCM (10 mL) was added dropwise to the solution at −78° C. The solution was stirred for an additional 30 min at −78° C. Then TEA (1.5 g, 14.59 mmol) was added to the resulting solution under −78° C. The resulting solution was warmed slowly to −30° C. over 20 min then quenched by the addition of AcOH (5 mL). The resulting solution was washed with water (20 mL) and dried over anhydrous Na₂SO₄. After filtration, the filtrate was concentrated under reduced pressure. The residue was purified by silica gel column chromatography, eluted with 50% acetonitrile in dichloromethane to afford 14-[[2-(2,6-dioxopiperidin-3-yl)-1,3-dioxo-2,3-dihydro-1H-isoindol-4-yl]amino]-3,6,9,12-tetraoxatetradecanal (1.4 g, 78%) as a yellow oil. ¹H NMR (400 MHz, CDCl₃) δ 9.75 (s, 1H), 8.14 (br s, 1H), 7.52 (dd, J=8.5, 7.2 Hz, 1H), 7.13 (d, J=7.2 Hz, 1H), 6.95 (d, J=8.5 Hz, 1H), 4.94 (dd, J=12.0, 5.3 Hz, 1H), 4.19 (s, 2H), 3.79-3.66 (m, 14H), 3.53-3.49 (m, 2H), 2.95-2.87 (m, 1H), 2.84-2.68 (m, 2H), 2.18-2.13 (m, 1H). LC/MS (ESI, m/z): [(M+1)]⁺=492.25.

### INTERMEDIATE B: tert-butyl 4-(4-(4,4,5,5-tetramethyl-1,3,2-dioxaborolan-2-yl)benzyl)piperazine-1-carboxylate

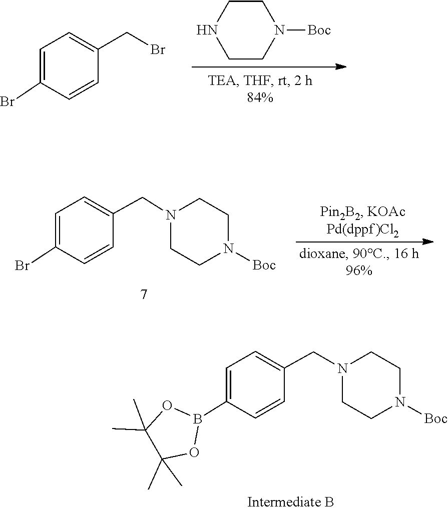 [SMILES: BrCc1ccc(Br)cc1 ; CC(C)(C)OC(=O)N1CCNCC1 ; CC(C)(C)OC(=O)N1CCN(Cc2ccc(Br)cc2)CC1 ; CC(C)(C)OC(=O)N1CCN(Cc2ccc(B3OC(C)(C)C(C)(C)O3)cc2)CC1]

### Step 1: tert-butyl 4-[(4-bromophenyl)methyl]piperazine-1-carboxylate

To a stirred solution of tert-butyl piperazine-1-carboxylate (32.8 g, 176 mmol) in THF (400 mL) were added TEA (44.5 mL, 439 mmol) and 1-bromo-4-(bromomethyl)benzene (40 g, 160 mmol) at 0° C. under nitrogen atmosphere. The resulting mixture was stirred for 2 h at room temperature and concentrated under reduced pressure. The residue was dissolved into DCM (100 mL) and washed with water (400 mL). The aqueous layer was extracted with DCM (2×200 mL). The combined organic layers was dried over anhydrous Na₂SO₄. After filtration, the filtrate was concentrated under reduced pressure to afford tert-butyl 4-[(4-bromophenyl)methyl]piperazine-1-carboxylate (48 g, 84%) as a white solid. ¹H NMR (400 MHz, DMSO-d₆) δ 7.53-7.41 (m, 2H), 7.30-7.15 (m, 2H), 3.45 (s, 2H), 3.33-3.22 (m, 4H), 2.36-2.09 (m, 4H), 1.35 (s, 9H). LC/MS (ESI, m/z): [(M+1)]⁺=355.00, 357.00

### Step 2: tert-butyl 4-[[4-(4,4,5,5-tetramethyl-1,3,2-dioxaborolan-2-yl)pheny]methyl]piperazine-1-carboxylate

To a solution of tert-butyl 4-[(4-bromophenyl)methyl]piperazine-1-carboxylate (46 g, 129 mmol) in 1,4-dioxane (400 mL) were added 4,4,5,5-tetramethyl-2-(4,4,5,5-tetramethyl-1,3,2-dioxaborolan-2-yl)-1,3,2-dioxaborolane (66 g, 259 mmol), Pd(dppf)Cl₂.CH₂Cl₂ (5.29 g, 6.47 mmol) and KOAc (25 g, 259 mmol) at room temperature under nitrogen atmosphere. The resulting mixture was stirred for an additional 16 h at 90° C. under N₂ atmosphere. The mixture was cooled to room temperature and diluted with water (500 mL). The resulting mixture was extracted with DCM (3×300 mL). The combined organic layers was washed with brine (400 mL) and dried over anhydrous Na₂SO₄. After filtration, the filtrate was concentrated under reduced pressure. The residue was purified by silica gel column chromatography, eluted with 10%-30% ethyl acetate in Petroleum ether to afford tert-butyl 4-[[4-(4,4,5,5-tetramethyl-1,3,2-dioxaborolan-2-yl)phenyl]methyl]piperazine-1-carboxylate (50 g, 96%) as a white solid. ¹H NMR (400 MHz, Chloroform-d) δ 7.80 (d, J=7.4 Hz, 2H), 7.36 (d, J=7.4 Hz, 2H), 3.55 (s, 2H), 3.44 (s, 4H), 2.40 (s, 4H), 1.47 (s, 9H), 1.36 (s, 12H). LC/MS (ESI, m/z): [(M+1)]⁺=403.35

### INTERMEDIATE C: trans-4-(5-bromo-2-chloro-7H-pyrrolo[2,3-d]pyrimidin-7-yl)cyclohexan-1-ol

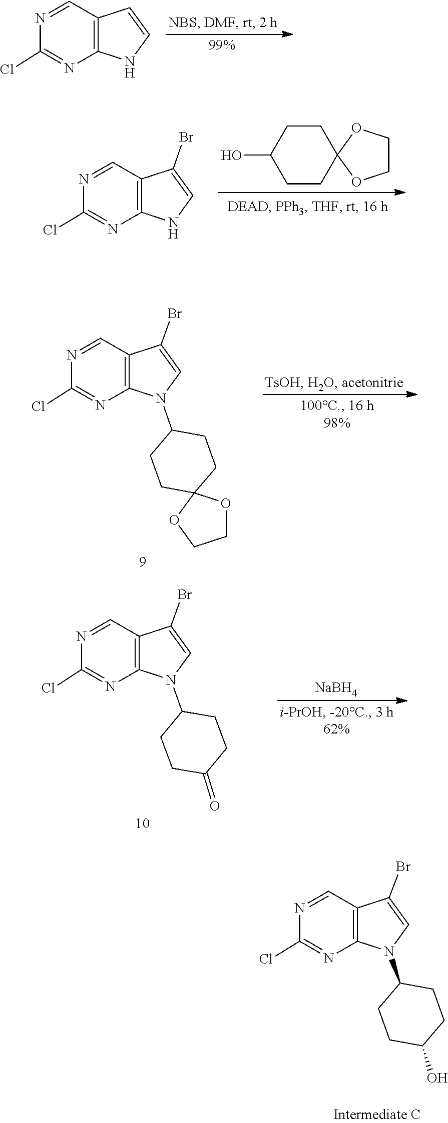

### Step 1: 5-Bromo-2-chloro-7H-pyrrolo[2,3-d]pyrimidine

To a stirred solution of 2-chloro-7H-pyrrolo[2,3-d]pyrimidine (20 g, 130 mmol) in DMF (300 mL) was added NBS (27.8 g, 156 mmol) at 0° C. under nitrogen atmosphere. The resulting solution was stirred for 2 h at room temperature. The resulting solution was poured into ice/water (1000 mL). Solid was precipitated and filtered. The filter cake was dried in a vacuum oven at room temperature to afford 5-bromo-2-chloro-7H-pyrrolo[2,3-d]pyrimidine (30 g, 99%) as a white solid. ¹H NMR (400 MHz, DMSO-d₆) δ 12.79 (br s, 1H), 8.86 (s, 1H), 7.90 (d, J=2.5 Hz, 1H). LC/MS (ESI, m/z): [(M+1)]⁺=231.95, 233.90, 235.95

### Step 2: 5-Bromo-2-chloro-7-[1,4-dioxaspiro[4.5]decan-8-yl]-7H-pyrrolo[2,3-d]pyrimidine

To a stirred solution of 5-bromo-2-chloro-7H-pyrrolo[2,3-d]pyrimidine (30 g, 129 mmol) in THF (1 L) were added 1,4-dioxaspiro[4.5]decan-8-ol (41 g, 258 mmol), PPh₃ (85 g, 323 mmol) and DEAD (56 g, 323 mmol) (over 30 min) at 0° C. The reaction was stirred at room temperature for 16 h. The resulting mixture was concentrated under reduced pressure. The residue was triturated with MeOH (200 mL) and filtered. The filter cake was washed with MeOH (3×50 mL) and dried to afford 5-bromo-2-chloro-7-[1,4-dioxaspiro[4.5]decan-8-yl]-7H-pyrrolo[2,3-d]pyrimidine (30 g, 62%) as a yellow solid. ¹H NMR (400 MHz, DMSO-d₆) δ 8.84 (s, 1H), 8.10 (s, 1H), 4.70 (tt, J=12.1, 3.9 Hz, 1H), 3.98-3.86 (m, 4H), 2.08 (qd, J=12.2, 5.3 Hz, 2H), 1.98-1.88 (m, 2H), 1.85-1.71 (m, 4H). LC/MS (ESI, m/z): [(M+1)]⁺=372.05, 374.05, 376.05

### Step 3: 4-[5-Bromo-2-chloro-7H-pyrrolo[2,3-d]pyrimidin-7-yl]cyclohexan-1-one

To a suspension of 5-bromo-2-chloro-7-[1,4-dioxaspiro[4.5]decan-8-yl]-7H-pyrrolo[2,3-d]pyrimidine (29 g, 77.8 mmol) in H₂O (500 mL) and acetonitrile (500 mL) was added 4-methylbenzenesulfonic acid (14 g, 81.7 mmol) at room temperature. The resulting mixture was stirred at 100° C. for 16 h. The mixture was cooled to room temperature and filtered. The filter cake was washed with water (3×30 mL) and dried in the air to afford 4-[5-bromo-2-chloro-7H-pyrrolo[2,3-d]pyrimidin-7-yl]cyclohexan-1-one (25 g, 98%) as a white solid. ¹H NMR (400 MHz, DMSO-d₆) δ 8.87 (s, 1H), 8.14 (s, 1H), 5.29-5.18 (m, 1H), 2.84-2.75 (m, 2H), 2.40-2.12 (m, 6H). LC/MS (ESI, m/z): [(M+1)]⁺=328.00, 330.00, 332.00

### Step 4: trans-4-[5-Bromo-2-chloro-7H-pyrrolo[2,3-d]pyrimidin-7-yl]cyclohexan-1-ol

To a stirred solution of 4-[5-bromo-2-chloro-7H-pyrrolo[2,3-d]pyrimidin-7-yl]cyclohexan-1-one (29 g, 88.3 mmol) in propan-2-ol (1 L) was added NaBH₄ (5.0 g, 132.4 mmol) in several portions at −20° C. under nitrogen atmosphere. The reaction was stirred at −20° C. for 3 h. The reaction was quenched by a saturated aqueous solution of NH₄Cl (100 mL) and concentrated under reduced pressure. The residue was taken up with water (200 mL) and ethyl acetate (200 mL). The organic layer was separated and dried over anhydrous Na₂SO₄. After filtration, the filtrate was concentrated under reduced pressure. The crude product (a mixture of trans:cis about 94:6) was separated by SFC with the following conditions: Column: CHIRALPAK IA-SFC-02, 5 cm×25 cm (5 um); Mobile Phase A: CO₂: 55%; Mobile Phase B: MeOH: 45%; Flow rate: 160 mL/min; Detector: 270 nm; RT 1: 5.41 min; RT2: 6.85 min. The faster eluting peak was collected and concentrated under reduced pressure to afford trans-4-[5-bromo-2-chloro-7H-pyrrolo[2,3-d]pyrimidin-7-yl]cyclohexan-1-ol (19 g, 62%) as a yellow solid. ¹H NMR (400 MHz, DMSO-d₆) δ 8.83 (s, 1H), 8.10 (s, 1H), 4.74 (d, J=4.3 Hz, 1H), 4.56 (td, J=10.4, 9.9, 5.0 Hz, 1H), 3.53 (ddt, J=11.0, 7.2, 4.0 Hz, 1H), 1.97-1.74 (m, 6H), 1.41 (qd, J=11.4, 5.1 Hz, 2H). LC/MS (ESI, m/z): [(M+1)]⁺=330.00, 332.00, 334.00

### INTERMEDIATE D: tert-butyl 4-((4-(4,4,5,5-tetramethyl-1,3,2-dioxaborolan-2-yl)phenyl)sulfonyl)piperazine-1-carboxylate

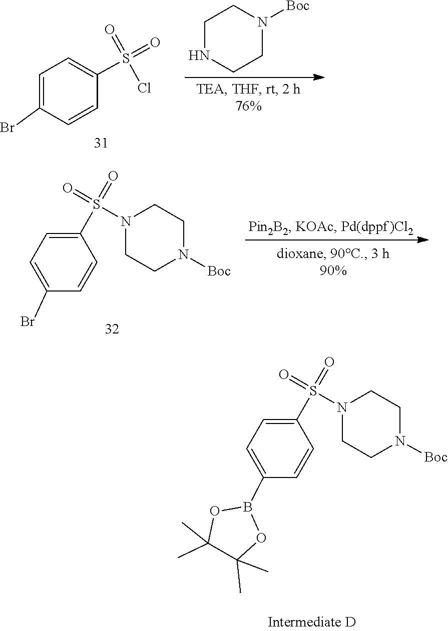 [SMILES: O=S(=O)(Cl)c1ccc(Br)cc1 ; CC(C)(C)OC(=O)N1CCNCC1 ; CC(C)(C)OC(=O)N1CCN(S(=O)(=O)c2ccc(Br)cc2)CC1 ; CC(C)(C)OC(=O)N1CCN(S(=O)(=O)c2ccc(B3OC(C)(C)C(C)(C)O3)cc2)CC1]

### Step 1: tert-butyl 4-(4-bromobenzenesulfonyl)piperazine-1-carboxylate

To a stirred solution of tert-butyl piperazine-1-carboxylate (12 g, 65 mmol) in THF (250 mL) were added TEA (12 g, 117 mmol) and 4-bromobenzene-1-sulfonyl chloride (15 g, 59 mmol). The resulting mixture was stirred for 2 h at room temperature and concentrated under reduced pressure. The residue was dissolved into DCM (200 mL), washed with water (100 mL) and 10% aq. HCl (50 mL). The organic layer was dried over anhydrous Na₂SO₄. After filtration, the filtrate was concentrated under reduced pressure to afford tert-butyl 4-(4-bromobenzenesulfonyl)piperazine-1-carboxylate (18 g, 76%) as a white solid. ¹H NMR (400 MHz, DMSO-d₆) δ 7.91-7.84 (m, 2H), 7.70-7.63 (m, 2H), 3.40 (t, J=5.0 Hz, 4H), 2.88 (t, J=5.1 Hz, 4H), 1.35 (s, 9H). LC/MS (ESI, m/z): [(M+18)]⁺=422.05, 424.05

### Step 2: tert-butyl 4-[4-(4,4,5,5-tetramethyl-1,3,2-dioxaborolan-2-yl)benzenesulfonyl]piperazine-1-carboxylate

To a solution of tert-butyl 4-(4-bromobenzenesulfonyl)piperazine-1-carboxylate (18 g, 44.41 mmol) in 1,4-dioxane (200 mL) were added 4,4,5,5-tetramethyl-2-(4,4,5,5-tetramethyl-1,3,2-dioxaborolan-2-yl)-1,3,2-dioxaborolane (34 g, 133.23 mmol), Pd(dppf)Cl₂.CH₂Cl₂ (3.63 g, 4.44 mmol) and KOAc (8.72 g, 88.82 mmol) at room temperature under nitrogen atmosphere. The resulting mixture was stirred for an additional 3 h at 90° C. The resulting mixture was cooled down to room temperature, diluted with water (200 mL) and extracted with DCM (3×200 mL). The combined organic layers was washed with brine (200 mL) and dried over anhydrous Na₂SO₄. After filtration, the filtrate was concentrated under reduced pressure. The residue was purified by silica gel column chromatography, eluted with 20%-50% ethyl acetate in petroleum ether to afford tert-butyl 4-[4-(4,4,5,5-tetramethyl-1,3,2-dioxaborolan-2-yl)benzenesulfonyl]piperazine-1-carboxylate (18 g, 90%) as a light pink solid. ¹H NMR (400 MHz, CDCl₃) δ 7.98 (d, J=7.7 Hz, 2H), 7.74 (d, J=7.7 Hz, 2H), 3.51 (t, J=5.1 Hz, 4H), 2.98 (t, J=4.9 Hz, 4H), 1.42 (s, 9H), 1.27 (s, 12H). LC/MS (ESI, m/z): [(M+23)]⁺=475.30.

### INTERMEDIATE E: 15-(1-(2,6-dioxopiperidin-3-yl)-3-methyl-2-oxo-2,3-dihydro-1H-benzo[d]imidazol-4-yl)-3,6,9,12-tetraoxapentadecanal

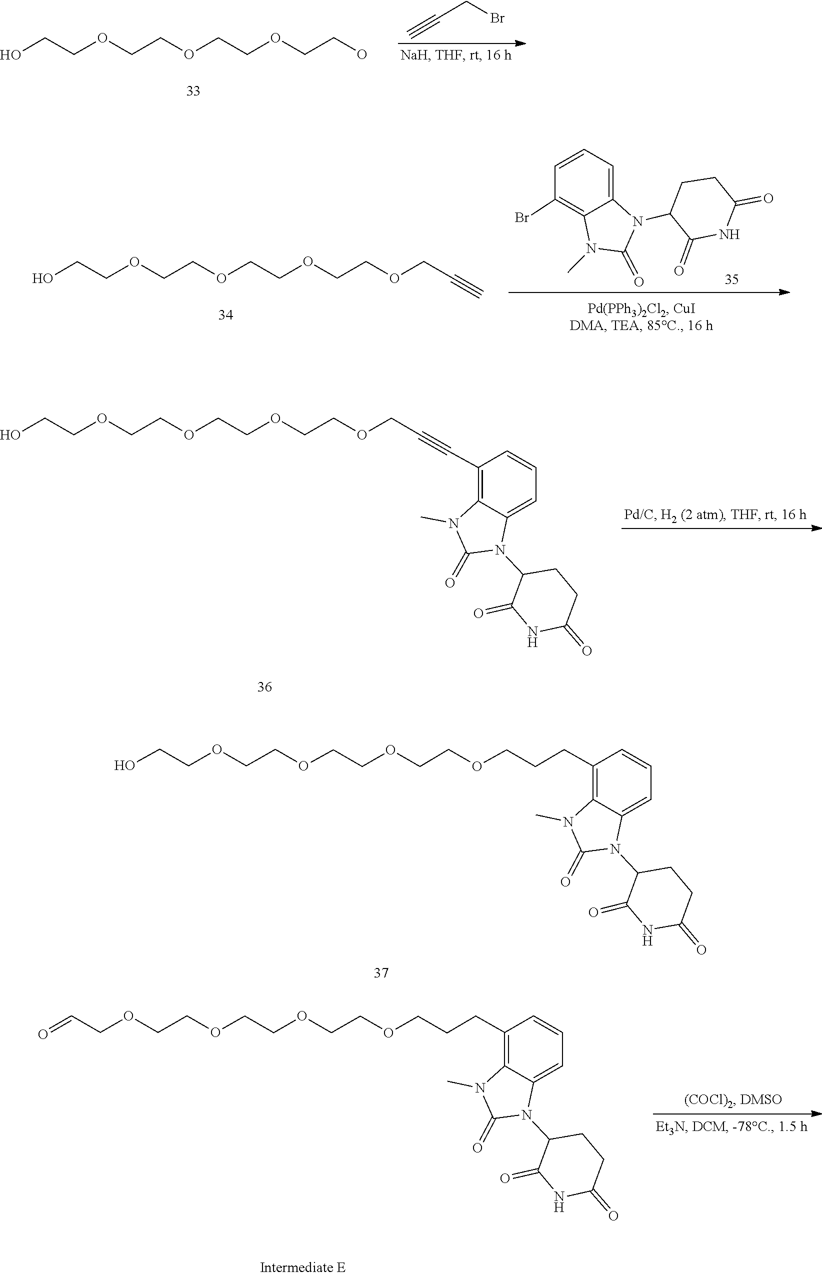

### Step 1: 3,6,9,12-Tetraoxapentadec-14-yn-1-ol

To a stirred solution of 2-[2-[2-(2-hydroxyethoxy)ethoxy]ethoxy]ethan-1-ol (29.9 g, 153.8 mmol) in THF (600 mL) was added NaH (60% dispersion in mineral oil, 6.2 g, 258 mmol) in portions over 5 min at 0° C. under nitrogen atmosphere followed by the addition of a solution of 3-bromoprop-1-yne (12.2 g, 103 mmol) in THF (20 mL) over 5 min. The resulting mixture was stirred for 16 h at room temperature under nitrogen atmosphere. The reaction was quenched with sat. NH₄Cl (200 mL) at 0° C. and extracted with EtOAc (2×500 mL). The combined organic layers was washed with brine (300 mL), dried over anhydrous Na₂SO₄. After filtration, the filtrate was concentrated under reduced pressure. The residue was purified by silica gel column chromatography, eluted with 10%-30% ethyl acetate in petroleum ether. The fractions containing desired product were collected and concentrated under reduced pressure to afford 3,6,9,12-tetraoxapentadec-14-yn-1-ol (5.6 g, 24%) as a light yellow oil. ¹H NMR (400 MHz, CDCl₃-d) δ 4.16 (q, J=1.8 Hz, 2H), 3.71-3.59 (m, 14H), 3.56 (ddt, J=5.7, 3.8, 1.7 Hz, 2H), 2.89-2.79 (m, 1H), 2.42 (t, J=2.4 Hz, 1H). LC/MS (ESI, m/z): [(M+1)]⁺=233.00

### Step 2: 3-[4-(1-Hydroxy-3,6,9,12-tetraoxapentadec-14-yn-15-yl)-3-methyl-2-oxo-2,3-dihydro-1H-1,3-benzodiazol-1-yl]piperidine-2,6-dione

To a stirred solution of 3-(4-bromo-3-methyl-2-oxo-2,3-dihydro-1H-1,3-benzodiazol-1-yl)piperidine-2,6-dione (1 g, 2.96 mmol) in DMA (10 mL) were added 3,6,9,12-tetraoxapentadec-14-yn-1-ol (2.75 g, 11.83 mmol), TEA (5 mL), CuI (56 mg, 0.30 mmol) and Pd(PPh₃)₂Cl₂ (207 mg, 0.30 mmol) at room temperature under nitrogen atmosphere. The resulting mixture was stirred for 16 h at 85° C. under nitrogen atmosphere. After cooling down to room temperature, the mixture was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 25%-45% B in 25 min; Flow rate: 80 mL/min; Detector: 220/254 nm; desired fractions were collected at 42% B and concentrated under reduced pressure to afford 3-[4-(1-hydroxy-3,6,9,12-tetraoxapentadec-14-yn-15-yl)-3-methyl-2-oxo-2,3-dihydro-1H-1,3-benzodiazol-1-yl]piperidine-2,6-dione (300 mg, 21%) as a white solid. ¹H NMR (400 MHz, DMSO-d₆) δ 11.12 (br s, 1H), 7.18 (d, J=7.8 Hz, 1H), 7.14 (dd, J=8.0, 1.1 Hz, 1H), 7.04 (t, J=7.9 Hz, 1H), 5.41 (dd, J=12.6, 5.4 Hz, 1H), 4.56 (t, J=5.5 Hz, 1H), 4.47 (s, 2H), 3.71-6.32 (m, 5H), 3.59 (dd, J=5.8, 3.3 Hz, 2H), 3.48 (q, J=4.7 Hz, 10H), 3.41 (t, J=5.4 Hz, 2H), 2.98-2.82 (m, 1H), 2.80-2.58 (m, 2H), 2.04 (ddd, J=11.0, 8.3, 5.3 Hz, 1H). LC/MS (ESI, m/z): [(M+1)]⁺=490.3

### Step 3: 3-[4-(1-Hydroxy-3,6,9,12-tetraoxapentadecan-15-yl)-3-methyl-2-oxo-2,3-dihydro-1H-1,3-benzodiazol-1-yl]piperidine-2,6-dione

To a stirred solution of 3-[4-(1-hydroxy-3,6,9,12-tetraoxapentadec-14-yn-15-yl)-3-methyl-2-oxo-2,3-dihydro-1H-1,3-benzodiazol-1-yl]piperidine-2,6-dione (200 mg, 0.41 mmol) in THF (10 mL) was added palladium on charcoal (20 mg, 10%, w/w) at room temperature. The resulting mixture was purged with H₂ for three times and was stirred for 16 h at room temperature under hydrogen atmosphere. Upon completion, the resulting mixture was filtered. The filter cake was washed with EtOAc (3×5 mL). The combined filtrates was concentrated under reduced pressure to afford 3-[4-(1-hydroxy-3,6,9,12-tetraoxapentadecan-15-yl)-3-methyl-2-oxo-2,3-dihydro-1H-1,3-benzodiazol-1-yl]piperidine-2,6-dione (200 mg, 99%) as a white solid. ¹H NMR (400 MHz, DMSO-d₆) δ 11.09 (br s, 1H), 7.00-6.94 (m, 2H), 6.89 (q, J=4.2 Hz, 1H), 5.37 (dd, J=12.6, 5.4 Hz, 1H), 4.56 (t, J=5.5 Hz, 1H), 4.04 (q, J=7.1 Hz, 2H), 3.57 (s, 3H), 3.56-3.52 (m, 8H), 3.50-3.44 (m, 4H), 3.41 (d, J=5.1 Hz, 2H), 3.01-2.92 (m, 2H), 2.92-2.84 (m, 1H), 2.77-2.58 (m, 2H), 2.06-1.96 (m, 1H), 1.89-1.78 (m, 2H), 1.18 (t, J=7.1 Hz, 2H). LC/MS (ESI, m/z): [(M+1)]⁺=494.4

### Step 4: 15-[1-(2,6-Dioxopiperidin-3-yl)-3-methyl-2-oxo-2,3-dihydro-1H-1,3-benzodiazol-4-yl]-3,6,9,12-tetraoxapentadecanal

To a stirred solution of oxalyl chloride (116 mg, 0.91 mmol) in DCM (10 mL) was added DMSO (95 mg, 1.22 mmol) dropwise at −78° C. under nitrogen atmosphere. The resulting solution was stirred for 30 min at −78° C. Then a solution of 3-[4-(1-hydroxy-3,6,9,12-tetraoxapentadecan-15-yl)-3-methyl-2-oxo-2,3-dihydro-1H-1,3-benzodiazol-1-yl]piperidine-2,6-dione (300 mg, 0.61 mmol) in DCM (5 mL) was added dropwise at −78° C. The resulting solution was stirred for another 30 min at the same temperature. Then TEA (307 mg, 3.04 mmol) was added at −78° C. The reaction temperature was slowly increased to room temperature in 30 min. The resulting mixture was diluted with water (20 mL), extracted with DCM (3×20 mL). The combined organic layers was dried over anhydrous Na₂SO₄. After filtration, the filtrate was concentrated under reduced pressure to afford crude 15-[1-(2,6-dioxopiperidin-3-yl)-3-methyl-2-oxo-2,3-dihydro-1H-1,3-benzodiazol-4-yl]-3,6,9,12-tetraoxapentadecanal (340 mg, purity >90%) as a yellow oil. LC/MS (ESI, m/z): [(M+1)]⁺=492.30

### INTERMEDIATE F: 15-(1-(2,6-dioxopiperidin-3-yl)-3-methyl-2-oxo-2,3-dihydro-1H-benzo[d]imidazol-5-yl)-3,6,9,12-tetraoxapentadecanal

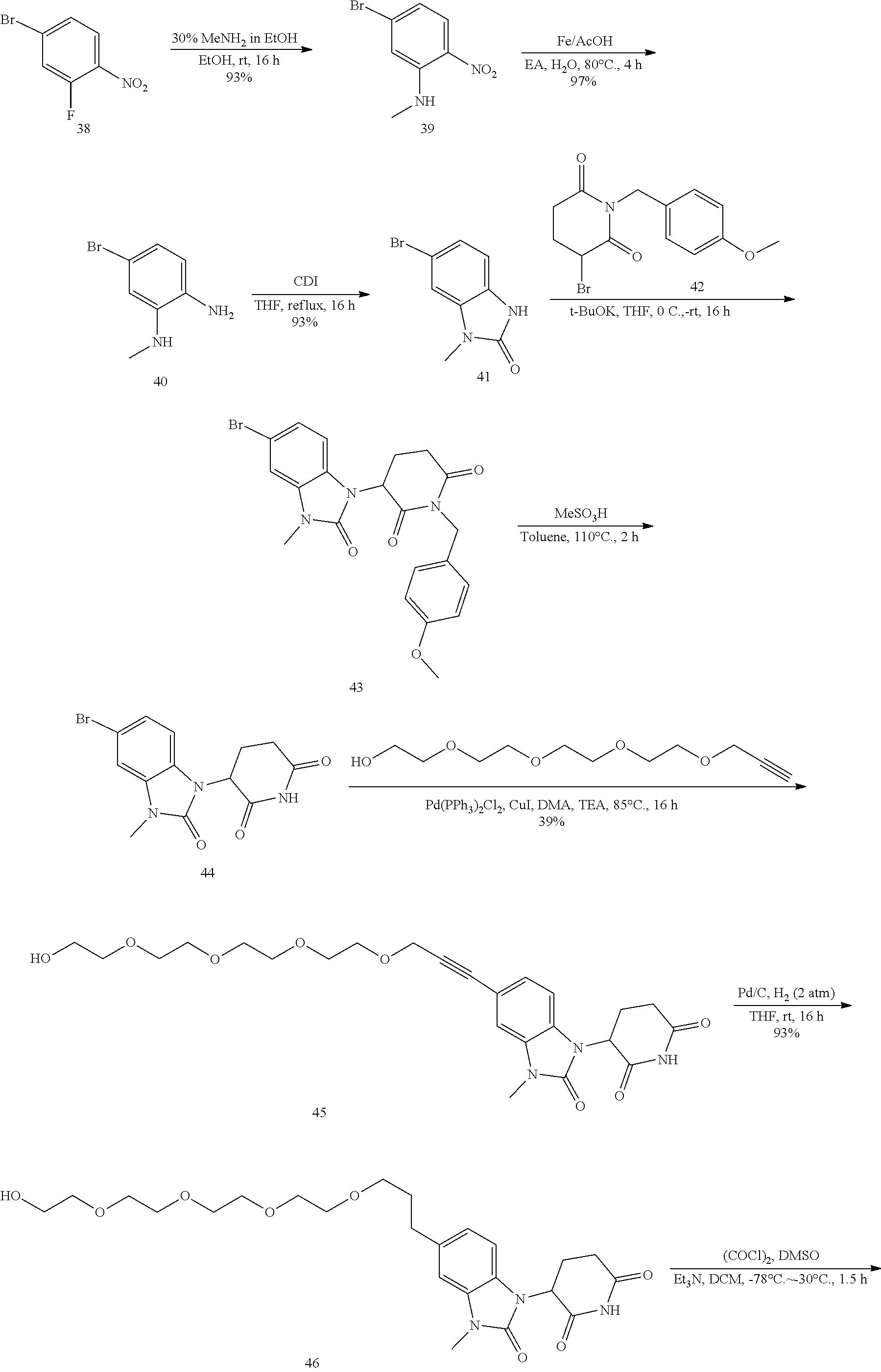

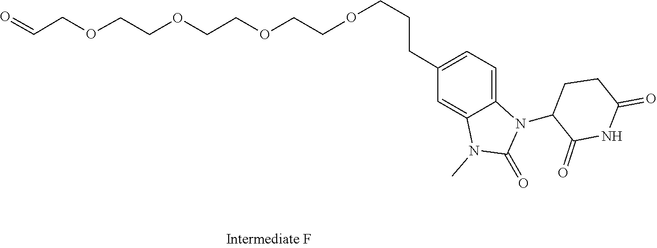 [SMILES: Cn1c(=O)n(C2CCC(=O)NC2=O)c2ccc(CCCOCCOCCOCCOCC=O)cc21]

### Step 1: 5-Bromo-N-methyl-2-nitroaniline

Into a sealed tube were added 4-bromo-2-fluoro-1-nitrobenzene (46 g, 209 mmol) and methylamine (30% in ethanol, 500 mL) at room temperature. After stirring for an additional 16 h, the resulting solution was concentrated under reduced pressure. The residue was triturated with water (1 L) and filtered. The filter cake was collected and dried under vacuum to afford 5-bromo-N-methyl-2-nitroaniline (45 g, 93%) as an orange solid. ¹H NMR (400 MHz, DMSO-d₆) δ 8.25 (br s, 1H), 8.02-7.94 (m, 1H), 7.17 (t, J=2.9 Hz, 1H), 6.83 (dd, J=9.1, 2.2 Hz, 1H), 2.95 (d, J=4.7 Hz, 3H). LC/MS (ESI, m/z): [(M+1)]⁺=231.15, 233.15

### Step 2: 5-Bromo-N1-methylbenzene-1,2-diamine

To a solution of 5-bromo-N-methyl-2-nitroaniline (45 g, 195 mmol) in EtOAc (600 mL) were added AcOH (200 mL), H₂O (20 mL) and Fe (45 g, 806 mmol) in portions at 50° C. under nitrogen atmosphere. The resulting mixture was stirred for 4 h at 80° C. After cooling to room temperature, the resulting mixture was filtered and the filtrate was diluted with water (1 L). The organic phase was separated and the aqueous phase was extracted with EtOAc (2×500 mL). The combined organic layers was washed with brine (2×400 mL) and dried over anhydrous Na₂SO₄. After filtration, the filtrate was concentrated under reduced pressure to afford 5-bromo-N1-methylbenzene-1,2-diamine (38 g, 97%) as a reddish brown oil. ¹H NMR (400 MHz, DMSO-d₆) δ 6.53 (dd, J=8.1, 2.2 Hz, 1H), 6.45 (d, J=8.1 Hz, 1H), 6.41 (d, J=2.2 Hz, 1H), 4.89 (q, J=5.1 Hz, 1H), 4.61 (s, 2H), 2.69 (d, J=4.2 Hz, 3H). LC/MS (ESI, m/z): [(M+1)]⁺=200.90, 202.90

### Step 3: 6-Bromo-1-methyl-2,3-dihydro-1H-1,3-benzodiazol-2-one

To a stirred solution of 5-bromo-N1-methylbenzene-1,2-diamine (38 g, 189 mmol) in THF (800 mL) was added CDI (37 g, 228 mmol) at room temperature under nitrogen atmosphere. The resulting mixture was refluxed for 16 h. The mixture was cooled to room temperature. The mixture was diluted with water (1 L) and stirred at room temperature for 30 min. The precipitated solids were collected by filtration and washed with water (200 mL). The filter cake was dried under vacuum to afford 6-bromo-1-methyl-2,3-dihydro-1H-1,3-benzodiazol-2-one (40 g, 93%) as a yellow solid. ¹H NMR (400 MHz, DMSO-d₆) δ 11.02 (br s, 1H), 7.35 (d, J=1.9 Hz, 1H), 7.14 (dd, J=8.2, 1.9 Hz, 1H), 6.92 (d, J=8.2 Hz, 1H), 3.27 (s, 3H). LC/MS (ESI, m/z): [(M+1)]⁺=227.10, 229.10

### Step 4: 3-(5-Bromo-3-methyl-2-oxo-2,3-dihydro-1H-1,3-benzodiazol-1-yl)-1-[(4-methoxyphenyl)methyl]piperidine-2,6-dione

A stirred solution of 6-bromo-1-methyl-2,3-dihydro-1H-1,3-benzodiazol-2-one (8.27 g, 36.42 mmol) in THF (180 mL) was treated with t-BuOK (5.70 g, 50.99 mmol) at 0° C. for 1 h under nitrogen atmosphere followed by the addition of 3-bromo-1-[(4-methoxyphenyl)methyl]piperidine-2,6-dione (11.4 g, 36.42 mmol). The resulting mixture was stirred for 16 h at room temperature under nitrogen atmosphere. The reaction was quenched with sat. NH₄Cl (200 mL) at 0° C. The resulting mixture was extracted with EtOAc (2×300 mL). The combined organic layers was washed with brine (200 mL) and dried over anhydrous Na₂SO₄. After filtration, the filtrate was concentrated under reduced pressure. The residue was purified by silica gel column chromatography, eluted with 50% ethyl acetate in dichloromethane to afford 3-(5-bromo-3-methyl-2-oxo-2,3-dihydro-1H-1,3-benzodiazol-1-yl)-1-[(4-methoxyphenyl)methyl]piperidine-2,6-dione (8 g, 48%) as a light yellow solid. ¹H NMR (400 MHz, DMSO-d₆) δ 7.48 (d, J=1.8 Hz, 1H), 7.29-7.08 (m, 3H), 7.01 (d, J=8.0 Hz, 1H), 6.86 (d, J=8.5 Hz, 2H), 5.54 (dd, J=13.0, 5.3 Hz, 1H), 4.79 (q, J=14.3 Hz, 2H), 3.73 (s, 3H), 3.35 (s, 3H), 3.12-2.98 (m, 1H), 2.86-2.66 (m, 2H), 2.12-2.02 (m, 1H). LC/MS (ESI, m/z): [(M+1)]⁺=458.06, 460.06.

### Step 5: 3-(5-Bromo-3-methyl-2-oxo-2,3-dihydro-1H-1,3-benzodiazol-1-yl)piperidine-2,6-dione

To a solution of 3-(5-bromo-3-methyl-2-oxo-2,3-dihydro-1H-1,3-benzodiazol-1-yl)-1-[(4-methoxyphenyl)methyl]piperidine-2,6-dione (8 g, 17.5 mmol) in toluene (40 mL) was added MeSO₃H (40 mL) at room temperature. The resulting mixture was stirred for 2 h at 110° C. under nitrogen atmosphere. After cooling to room temperature, the resulting mixture was concentrated under reduced pressure and the residue was diluted with ice/water (200 mL). The precipitated solids were collected by filtration, washed with hexane (3×10 mL) and dried under reduced pressure to afford 3-(5-bromo-3-methyl-2-oxo-2,3-dihydro-1H-1,3-benzodiazol-1-yl)piperidine-2,6-dione (3.6 g, 61%) as a light yellow solid. ¹H NMR (400 MHz, DMSO-d₆) δ 11.13 (br s, 1H), 7.48 (d, J=1.9 Hz, 1H), 7.22 (dd, J=8.4, 1.9 Hz, 1H), 7.11 (d, J=8.4 Hz, 1H), 5.39 (dd, J=12.8, 5.3 Hz, 1H), 3.35 (s, 3H), 2.96-2.82 (m, 1H), 2.79-2.58 (m, 2H), 2.07-1.98 (m, 1H). LC/MS (ESI, m/z): [(M+1)]⁺=337.95, 339.95

### Step 6: 3-[5-(1-Hydroxy-3,6,9,12-tetraoxapentadec-14-yn-15-yl)-3-methyl-2-oxo-2,3-dihydro-1H-1,3-benzodiazol-1-yl]piperidine-2,6-dione

To a stirred solution of 3-(5-bromo-3-methyl-2-oxo-2,3-dihydro-1H-1,3-benzodiazol-1-yl)piperidine-2,6-dione (350 mg, 1.04 mmol.) in DMSO (4 mL) were added 3,6,9,12-tetraoxapentadec-14-yn-1-ol (962 mg, 4.14 mmol), TEA (2 mL), CuI (16 mg, 0.083 mmol) and Pd(PPh₃)₄ (120 mg, 0.11 mmol) at room temperature. The resulting mixture was stirred for 16 h at 85° C. under nitrogen atmosphere. The reaction mixture was cooled to room temperature and purified by reverse phase flash chromatography with the following conditions: Column: WelFlash™ C18-I, 20-40 μm, 330 g; Eluent A: Water (plus 10 mmol/L HCOOH); Eluent B: ACN; Gradient: 65%-85% B in 25 min; Flow rate: 80 mL/min; Detector: 220/254 nm; desired fractions were collected at 73% B and concentrated under reduced pressure to afford 3-[5-(1-hydroxy-3,6,9,12-tetraoxapentadec-14-yn-15-yl)-3-methyl-2-oxo-2,3-dihydro-1H-1,3-benzodiazol-1-yl]piperidine-2,6-dione (200 mg, 39%) as a light yellow solid. ¹H NMR (400 MHz, DMSO-d₆) δ 11.12 (s, 1H), 7.34 (d, J=1.4 Hz, 1H), 7.24-7.08 (m, 2H), 5.39 (dd, J=12.7, 5.4 Hz, 1H), 4.57 (t, J=5.5 Hz, 1H), 4.40 (s, 2H), 3.67-3.62 (m, 2H), 3.61-3.56 (m, 2H), 3.53 (d, J=6.4 Hz, 8H), 3.50-3.46 (m, 2H), 3.41 (ddd, J=5.8, 4.9, 1.1 Hz, 2H), 3.35 (s, 3H), 2.90 (ddd, J=16.6, 12.4, 5.0 Hz, 1H), 2.78-2.58 (m, 2H), 2.04 (ddd, J=10.3, 5.5, 3.5 Hz, 1H). LC/MS (ESI, m/z): [(M+1)]⁺=490.35

### Step 7: 3-[5-(1-Hydroxy-3,6,9,12-tetraoxapentadecan-15-yl)-3-methyl-2-oxo-2,3-dihydro-1H-1,3-benzodiazol-1-yl]piperidine-2,6-dione

To a stirred solution of 3-[5-(1-hydroxy-3,6,9,12-tetraoxapentadec-14-yn-15-yl)-3-methyl-2-oxo-2,3-dihydro-1H-1,3-benzodiazol-1-yl]piperidine-2,6-dione (200 mg, 0.41 mmol) in THF (10 mL) was added palladium on charcoal (20 mg, 10%, w/w) at room temperature. The resulting mixture was purged with H₂ for three times and was stirred for 3 h at room temperature under hydrogen atmosphere. Upon completion, the resulting mixture was filtered. The filter cake was washed with EtOAc (3×5 mL). The combined filtrates was concentrated under reduced pressure to afford 3-[5-(1-hydroxy-3,6,9,12-tetraoxapentadecan-15-yl)-3-methyl-2-oxo-2,3-dihydro-1H-1,3-benzodiazol-1-yl]piperidine-2,6-dione (200 mg, 99%) as a yellow solid. ¹H NMR (400 MHz, DMSO-d₆) δ 11.09 (s, 1H), 7.07-6.98 (m, 2H), 6.88 (dd, J=8.1, 1.6 Hz, 1H), 5.34 (dd, J=12.7, 5.5 Hz, 1H), 4.57 (t, J=5.5 Hz, 1H), 4.04 (q, J=7.1 Hz, 1H), 3.57-3.49 (m, 12H), 3.40 (ddd, J=6.5, 4.8, 2.6 Hz, 4H), 2.98-2.83 (m, 1H), 2.78-2.57 (m, 4H), 2.00 (s, 3H), 1.88-1.73 (m, 2H), 1.18 (t, J=7.1 Hz, 2H). LC/MS (ESI, m/z): [(M+1)]⁺=494.40

### Step 8: 15-[1-(2,6-Dioxopiperidin-3-yl)-3-methyl-2-oxo-2,3-dihydro-1H-1,3-benzodiazol-5-yl]-3,6,9,12-tetraoxapentadecanal

To a stirred solution of oxalyl chloride (77 mg, 0.61 mmol) in DCM (5 mL) was added DMSO (63 mg, 0.81 mmol) dropwise at −78° C. under nitrogen atmosphere. The solution was allowed to react for 30 min at the same temperature. Then a solution of 3-[5-(1-hydroxy-3,6,9,12-tetraoxapentadecan-15-yl)-3-methyl-2-oxo-2,3-dihydro-1H-1,3-benzodiazol-1-yl]piperidine-2,6-dione (200 mg, 0.41 mmol) in DCM (5 mL) was added dropwise to the reaction at −78° C. The solution was stirred for another 30 min at the same temperature. Then TEA (205 mg, 2.01 mmol) was added to the reaction at −78° C. The resulting solution was slowly warmed to −30° C. over 30 min. The resulting mixture was diluted with water (20 mL) and extracted with DCM (3×20 mL). The combined organic layers was dried over anhydrous Na₂SO₄. After filtration, the filtrate was concentrated under reduced pressure to give crude 15-[1-(2,6-dioxopiperidin-3-yl)-3-methyl-2-oxo-2,3-dihydro-1H-1,3-benzodiol-5-yl]-3,6,9,12-tetraoxapentadecanal (190 mg) as a light yellow oil.

LC/MS (ESI, m/z): [(M+1)]⁺=492.30

### INTERMEDIATE G: Trans-4-(6-(butylamino)-3-(4-(piperazin-1-ylsulfonyl)phenyl)-1H-pyrazolo[3,4-d]pyrimidin-1-yl)cyclohexan-1-ol hydrochloride

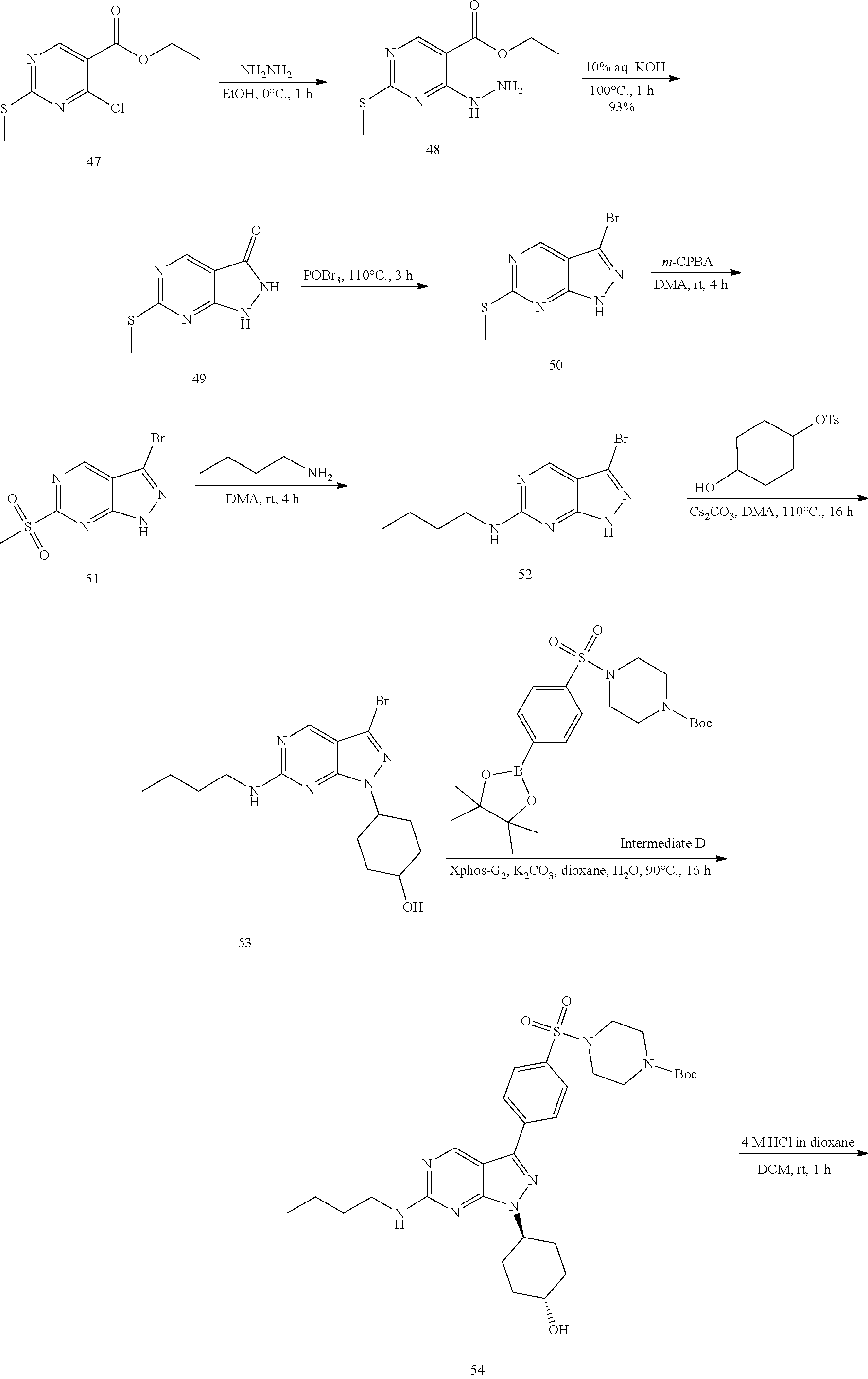

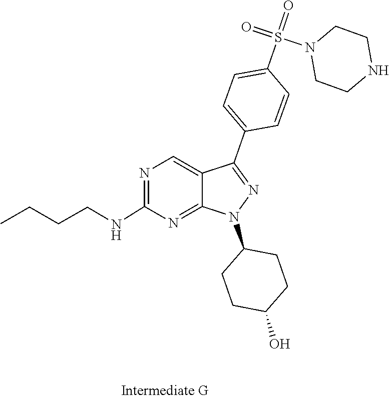 [SMILES: CCCCNc1ncc2c(-c3ccc(S(=O)(=O)N4CCNCC4)cc3)nn([C@H]3CC[C@H](O)CC3)c2n1]

Step 1: Ethyl 4-hydrazinyl-2-(methylthio)pyrimidine-5-carboxylate

To a stirred solution of hydrazine hydrate (32.9 g, 645 mmol) in EtOH (500 mL) was added a solution of ethyl 4-chloro-2-(methylsulfanyl)pyrimidine-5-carboxylate (50 g, 215 mmol) in EtOH (1.5 L) dropwise at 0° C. After stirring for an additional 1 h at 0° C., the mixture was concentrated under reduced pressure to give ethyl 4-hydrazinyl-2-(methylsulfanyl)pyrimidine-5-carboxylate (49 g, 100%) as a white solid. ¹H NMR (400 MHz, DMSO-d₆) δ 8.50 (s, 1H), 4.27 (t, J=7.1 Hz, 2H), 2.53 (s, 3H), 1.30 (t, J=7.1 Hz, 3H). LC/MS (ESI, m/z): [(M+1)]⁺=229.15.

### Step 2: 6-(Methylsulfanyl)-1H,2H,3H-pyrazolo[3,4-d]pyrimidin-3-one

To a stirred solution of potassium hydroxide (50 g, 891 mmol) in H₂O (900 mL) was added ethyl 4-hydrazinyl-2-(methylsulfanyl)pyrimidine-5-carboxylate (49 g, 214 mmol) at room temperature. The resulting mixture was stirred for 1 h at 100° C. The resulting mixture was cooled down to room temperature. The mixture was acidified to pH=5 with AcOH. The precipitated solids were collected by filtration, washed with water (2×200 mL) and dried to afford 6-(methylsulfanyl)-1H,2H,3H-pyrazolo[3,4-d]pyrimidin-3-one (36.5 g, 93%) as a yellow solid. ¹H NMR (400 MHz, DMSO-d₆) δ 11.76 (br s, 2H), 8.82 (s, 1H), 2.52 (s, 3H). LC/MS (ESI, m/z): [(M +1)]⁺=183.25

### Step 3: 3-Bromo-6-(methylthio)-1H-pyrazolo[3,4-d]pyrimidine

A mixture of 6-(methylsulfanyl)-1H,2H,3H-pyrazolo[3,4-d]pyrimidin-3-one (45 g, 247 mmol) and phosphoroyl tribromide (566 g, 1.97 mol) was stirred for 3 h at 110° C. under nitrogen atmosphere. After cooling down to room temperature, the resulting mixture was triturated with ethyl ether (500 mL). The solids were collected by filtration and washed with ethyl ether (2×100 mL). The filter cake was then triturated with ice/water (3 L). The pH value of the mixture was adjusted to pH =5 with concentrated aqueous solution of ammonia. The resulting mixture was filtered. The filter cake was washed with water (3×200 mL) and dried to afford 3-bromo-6-(methylsulfanyl)-1H-pyrazolo[3,4-d]pyrimidine (55 g, 91%) as a light yellow solid. ¹H NMR (400 MHz, DMSO-d₆) δ 14.22 (br s, 1H), 9.01 (s, 1H), 2.57 (s, 3H). LC/MS (ESI, m/z): [(M+1)]⁺=245.15, 247.15

### Step 4: 3-Bromo-6-(methylsulfonyl)-1H-pyrazolo[3,4-d]pyrimidine

To a stirred solution of 3-bromo-6-(methylsulfanyl)-1H-pyrazolo[3,4-d]pyrimidine (65 g, 265 mmol) in DMA (650 mL) was added m-CPBA (162 g, 796 mmol) in portions at 0° C. under nitrogen atmosphere. The resulting solution was stirred for 4 h at room temperature. The solution was used to next step directly. LC/MS (ESI, m/z): [(M+1)]⁺=277.10, 279.10

### Step 5: 3-Bromo-N-butyl-1H-pyrazolo[3,4-d]pyrimidin-6-amine

To the above solution was added butan-1-amine (120 g, 1.64 mol) dropwise at 0° C. under nitrogen atmosphere. The solution was stirred for 16 h at room temperature. The reaction was diluted with water (1.3 L). The precipitated solids were collected by filtration, washed with water (2×200 mL) and dried to afford 3-bromo-N-butyl-1H-pyrazolo[3,4-d]pyrimidin-6-amine (57 g, 90%) as a light yellow solid. ¹H NMR (400 MHz, DMSO-d₆) δ 13.06 (br s, 1H), 8.64 (s, 1H), 7.70 (br s, 1H), 3.39-3.28 (m, 2H), 1.54 (p, J=7.4 Hz, 2H), 1.34 (h, J=7.4 Hz, 2H), 0.90 (t, J=7.3 Hz, 3H). LC/MS (ESI, m/z): [(M+1)]⁺=270.20, 272.20

### Step 6: 4-(3-Bromo-6-(butylamino)-1H-pyrazolo[3,4-d]pyrimidin-1-yl)cyclohexan-1-ol

To a solution of 3-bromo-N-butyl-1H-pyrazolo[3,4-d]pyrimidin-6-amine (16 g, 59 mmol) in DMA (250 mL) were added 4-hydroxycyclohexyl 4-methylbenzene-1-sulfonate (48 g, 178 mmol) and Cs₂CO₃ (58 g, 178 mmol) at room temperature. The resulting mixture was stirred for 16 h at 110° C. under nitrogen atmosphere. The mixture was cooled down to room temperature, diluted with water (1 L) and extracted with EtOAc (2×500 mL). The combined organic layers was washed with brine (300 mL) and dried over anhydrous Na₂SO₄. After filtration, the filtrate was concentrated under reduced pressure. The residue was purified by silica gel column chromatography, eluted with 25% ethyl acetate in petroleum ether to afford 4-[3-bromo-6-(butylamino)-1H-pyrazolo[3,4-d]pyrimidin-1-yl]cyclohexan-1-ol (18 g, 83%) as a white solid. ¹H NMR (400 MHz, DMSO-d₆) δ 8.64 (s, 1H), 7.73 (br s, 1H), 4.74-4.35 (m, 2H), 3.90-3.44 (m, 1H), 3.40-3.25 (m, 2H), 2.37-2.15 (m, 1H), 2.04-1.71 (m, 4H), 1.68-1.45 (m, 4H), 1.42-1.23 (m, 3H), 1.00-0.81 (m, 3H). LC/MS (ESI, m/z): [(M+1)]⁺=368.15, 370.15

### Step 7: tert-butyl 4-((4-(6-(butylamino)-1-(trans-4-hydroxycyclohexyl)-1H-pyrazolo[3,4-d]pyrimidin-3-yl)phenyl)sulfonyl)piperazine-1-carboxylate

To a solution of 4-[3-bromo-6-(butylamino)-1H-pyrazolo[3,4-d]pyrimidin-1-yl]cyclohexan-1-ol (5.5 g, 14.9 mmol) in 1,4-dioxane (55 mL) and H₂O (11 mL) were added tert-butyl 4-[4-(4,4,5,5-tetramethyl-1,3,2-dioxaborolan-2-yl)benzenesulfonyl]piperazine-1-carboxylate (8.1 g, 17.9 mmol), K₂CO₃ (6.2 g, 44.8 mmol) and XPhos-G2 pre-catalyst (1.2 g, 1.49 mmol, CAS No.: 1310584-14-5) at room temperature under nitrogen atmosphere. The resulting mixture was stirred for 3 h at 90° C. The mixture was cooled down to room temperature and diluted with ethyl acetate (50 mL). The resulting mixture was filtered. The filter cake was washed with ethyl acetate (2×30 mL). The combined filtrates was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 45%-70% B in 30 min; Flow rate: 80 mL/min; Detector: 220/254 nm; desired fractions were collected at 65% B and concentrated under reduced pressure to afford tert-butyl 4-[4-[6-(butylamino)-1-[trans-4-hydroxycyclohexyl]-1H-pyrazolo[3,4-d]pyrimidin-3-yl]benzenesulfonyl]piperazine-1-carboxylate (1.9 g, 21%) as a white solid. ¹H NMR (400 MHz, CDCl₃) δ 8.96 (s, 1H), 8.08 (d, J=8.2 Hz, 2H), 7.87 (d, J=8.2 Hz, 2H), 5.63 (br s, 1H), 4.80-4.60 (m, 1H), 3.93-3.78 (m, 1H), 3.55 (t, J=5.1 Hz, 4H), 3.04 (t, J=5.1 Hz, 4H), 2.33-2.15 (m, 4H), 2.15-2.01 (m, 2H), 1.73-1.44 (m, 6H), 1.43 (s, 9H), 1.02 (t, J=7.3 Hz, 3H). LC/MS(ESI, m/z): [(M+1)]⁺=614.40

### Step 8: trans-4-(6-(butylamino)-3-(4-(piperazin-1-ylsulfonyl)phenyl)-1H-pyrazolo[3,4-d]pyrimidin-1-yl)cyclohexan-1-ol hydrochloride

To a stirred solution of tert-butyl 4-[4-[6-(butylamino)-1-[trans-4-hydroxycyclohexyl]-1H-pyrazolo[3,4-d]pyrimidin-3-yl]benzenesulfonyl]piperazine-1-carboxylate (1.9 g, 3.1 mmol) in DCM (80 mL) was added a solution of hydrochloride in 1,4-dioxane (4 M, 40 mL) dropwise at 0° C. under nitrogen atmosphere. The solution was stirred for 1 h at room temperature. The resulting solution was concentrated under reduced pressure to afford trans-4-[6-(butylamino)-3-[4-(piperazine-1-sulfonyl)phenyl]-1H-pyrazolo[3,4-d]pyrimidin-1-yl]cyclohexan-1-ol hydrochloride (1.7 g, 100%) as an off white solid. ¹H NMR (400 MHz, DMSO-d₆) δ 9.31 (s, 2H), 8.28 (d, J=8.2 Hz, 2H), 7.88 (d, J=8.2 Hz, 2H), 6.88 (br s, 3H), 4.62-4.47 (m, 1H), 3.76-3.35 (m, 6H), 3.25-3.11 (m, 4H), 2.16-1.87 (m, 6H), 1.58 (q, J=7.4 Hz, 2H), 1.47-1.32 (m, 4H), 0.94 (t, J=7.4 Hz, 3H). LC/MS (ESI, m/z): [(M+1)]⁺=514.30

### INTERMEDIATE H: 3-(4-[3-[3-(3-Aminopropoxy)propoxy]propyl]-3-methyl-2-oxo-2,3-dihydro-1H-1,3-benzodiazol-1-yl)piperidine-2,6-dione hydrochloride

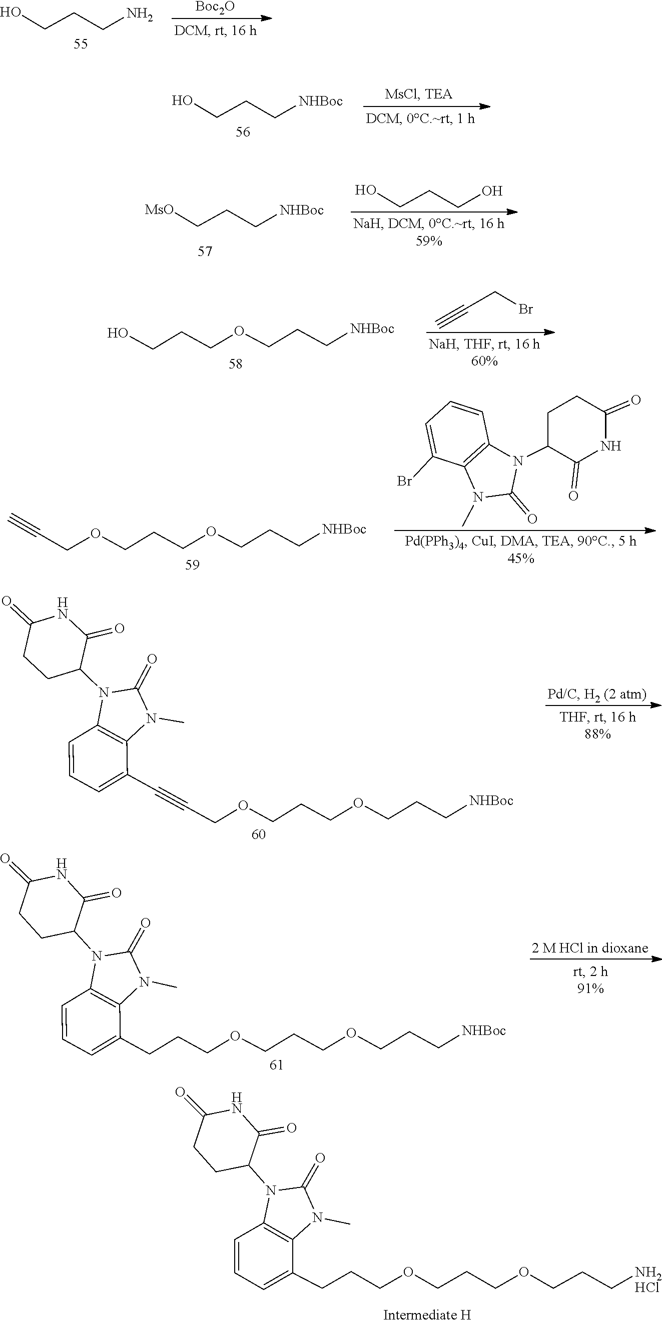

Step 1: tert-butyl N-(3-hydroxypropyl)carbamate

To a stirred solution of 3-aminopropan-1-ol (102 g, 1.35 mol) in DCM (1.5 L) was added a solution of Boc₂O (311 g, 1.43 mol) in DCM (500 mL) dropwise at room temperature under nitrogen atmosphere. The resulting solution was stirred for 4 hours at room temperature under nitrogen atmosphere. The resulting solution was concentrated under reduced pressure. The resulting mixture was diluted with water (2 L) and extracted with CH₂Cl₂ (3×1 L). The combined organic layers was washed with brine (500 mL), dried over anhydrous Na₂SO₄. After filtration, the filtrate was concentrated under reduced pressure. This resulted in tert-butyl N-(3-hydroxypropyl) carbamate (260 g, 82%) as a light yellow oil. ¹H NMR (400 MHz, CDCl₃) δ 5.03 (br, 1H), 3.61 (dd, J=6.9, 4.5 Hz, 2H), 3.22 (q, J=4.6, 2.8 Hz, 2H), 1.63 (p, J=5.9 Hz, 2H), 1.43-1.35 (m, 9H)

### Step 2: tert-butyl N-[3-(methanesulfonyloxy)propyl]carbamate

To a stirred solution of tert-butyl N-(3-hydroxypropyl)carbamate (92.6 g, 528.4 mmol) in DCM (800 mL) were added TEA (80.2 g, 792.7 mmol) and a solution of MsCl (60.5 g, 528.5 mmol) in DCM (200 mL) dropwise at 0° C. over 30 min under nitrogen atmosphere. The resulting mixture was stirred for 30 min at room temperature under nitrogen atmosphere. The resulting mixture was diluted with water (2 L) and extracted with CH₂Cl₂ (3×800 mL). The combined organic layers was washed with brine (1 L) and dried over anhydrous Na₂SO₄. After filtration, the filtrate was concentrated under reduced pressure. The residue was purified by silica gel column chromatography, eluted with 40% ethyl acetate in petroleum ether to afford tert-butyl N-[3-(methanesulfonyloxy)propyl]carbamate (112 g, 84%) as a light brown solid. ¹H NMR (400 MHz, CDCl₃) δ 4.91 (br s, 1H), 4.24 (td, J=6.9, 6.4, 2.9 Hz, 2H), 3.21 (d, J=7.9 Hz, 2H), 3.06-2.92 (m, 3H), 1.89 (pd, J=6.3, 2.6 Hz, 2H), 1.39 (d, J=3.0 Hz, 9H)

### Step 3: tert-butyl N-[3-(3-hydroxypropoxy)propyl]carbamate

To a solution of propane-1,3-diol (50 g, 657 mmol) in DMF (500 mL) was added sodium hydride (4.79 g, 200 mmol, 60% dispersed in mineral oil) at 0° C. The mixture was stirred for 15 min at room temperature. To the above mixture was added a solution of tert-butyl N-[3-(methanesulfonyloxy)propyl]carbamate (25.3 g, 100 mmol) in DMF (150 mL) dropwise at room temperature. The mixture was stirred for 16 hours at room temperature. The reaction was quenched with sat. NH₄Cl (200 mL) at 0° C. The resulting mixture was concentrated under reduced pressure. The residue was diluted with brine (1.5 L) and extracted with EtOAc (3×500 mL). The combined organic layers was washed with brine (500 mL) and dried over anhydrous Na₂SO₄. After filtration, the filtrate was concentrated under reduced pressure. The residue was purified by silica gel column chromatography, eluted with 30% ethyl acetate in petroleum ether to afford tert-butyl N-[3-(3-hydroxypropoxy)propyl]carbamate (13.7 g, 59%) as a light yellow oil. ¹H NMR (400 MHz, CDCl₃) δ 4.11 (s, 1H), 3.57 (td, J=6.3, 1.9 Hz, 2H), 3.50-3.42 (m, 4H), 3.19 (q, J=6.5 Hz, 2H), 1.88-1.78 (m, 2H), 1.71 (p, J=6.5 Hz, 2H), 1.41 (s, 9H). LC/MS (ESI, m/z): [(M+1]⁺=234.15

### Step 4: tert-butyl N-[3-[3-(prop-2-yn-1-yloxy)propoxy]propyl]carbamate

To a solution of tert-butyl N-[3-(3-hydroxypropoxy)propyl]carbamate (12.5 g, 53.6 mmol) in THF (300 mL) was added sodium hydride (2.6 g, 108.3 mmol, 60% dispersion in mineral oil) at 0° C. The mixture was stirred for 15 min. To the mixture was added a solution of 3-bromoprop-1-yne (6.4 g, 53.8 mmol) in THF (50 mL) dropwise and the mixture was warmed to room temperature and stirred for 16 hours. The reaction was quenched with sat. NH₄Cl (200 mL) at 0° C., diluted with brine (500 mL) and extracted with EtOAc (3×300 mL). The combined organic layers was washed with brine (500 mL) and dried over anhydrous Na₂SO₄. After filtration, the filtrate was concentrated under reduced pressure. The residue was purified by silica gel column chromatography, eluted with 25% ethyl acetate in petroleum ether to afford tert-butyl N-[3-[3-(prop-2-yn-1-yloxy)propoxy]propyl]carbamate (9.2 g, 60%) as a light yellow solid. ¹H NMR (400 MHz, CDCl₃) δ 4.90 (br s, 1H), 4.11 (d, J=2.4 Hz, 2H), 3.57 (t, J=6.3 Hz, 2H), 3.50-3.40 (m, 4H), 3.18 (t, J=7.2 Hz, 2H), 2.42 (t, J=2.4 Hz, 1H), 1.83 (p, J=6.2 Hz, 2H), 1.71 (p, J=6.2 Hz, 2H), 1.41 (s, 9H)

### Step 5: tert-butyl N-[3-[3-([3-[1-(2,6-dioxopiperidin-3-yl)-3-methyl-2-oxo-2,3-dihydro-1H-1,3-benzodiazol-4-yl]prop-2-yn-1-yl]oxy)propoxy]propyl]carbamate

To a solution of 3-(4-bromo-3-methyl-2-oxo-2,3-dihydro-1H-1,3-benzodiazol-1-yl)piperidine-2,6-dione (2 g, 5.91 mmol) in DMA (30 mL) were added tert-butyl N-[3-[3-(prop-2-yn-1-yloxy)propoxy]propyl]carbamate (3 g, 11.06 mmol), TEA (15 mL), CuI (113 mg, 0.59 mmol) and Pd(PPh₃)₄ (0.68 g, 0.59 mmol) at room temperature under nitrogen atmosphere. The resulting mixture was stirred for 5 h at 90° C. under nitrogen atmosphere. The resulting mixture was cooled down to room temperature and concentrated under reduced pressure. The residue was purified by reverse phase flash chromatography with the following conditions: Column: WelFlash™ C18-I, 20-40 μm, 330 g; Eluent A: Water (plus 10 mmol/L HCOOH); Eluent B: ACN; Gradient: 35%-60% B in 15 min; Flow rate: 80 mL/min; Detector: 220/254 nm; desired fractions were collected at 55% B and concentrated under reduced pressure to afford tert-butyl N-[3-[3-([3-[1-(2,6-dioxopiperidin-3-yl)-3-methyl-2-oxo-2,3-dihydro-1H-1,3-benzodiol-4-yl]prop-2-yn-1-yl]oxy)propoxy]propyl]carbamate (1.4 g, 45%) as a light brown solid. ¹H NMR (400 MHz, DMSO-d₆) δ 11.12 (br s, 1H), 7.19-7.12 (m, 2H), 7.08-6.95 (m, 1H), 6.75 (br s, 1H), 5.41 (dd, J=12.7, 5.4 Hz, 1H), 4.43 (s, 2H), 3.64 (s, 3H), 3.63-3.55 (m, 4H), 3.42 (t, J=6.4 Hz, 2H), 3.35 (d, J=12.6 Hz, 2H), 3.00-2.83 (m, 3H), 2.71 (m, 2H), 2.10-1.97 (m, 2H), 1.78 (p, J=6.4 Hz, 2H), 1.59 (p, J=6.6 Hz, 2H), 1.37 (s, 9H). LC/MS (ESI, m/z): [(M+1)]⁺=529.35

### Step 6: tert-butyl N-[3-(3-[3-[1-(2,6-dioxopiperidin-3-yl)-3-methyl-2-oxo-2,3-dihydro-1H-1,3-benzodiazol-4-yl]propoxy]propoxy)propyl]carbamate

To a stirred solution of tert-butyl N-[3-[3-([3-[1-(2,6-dioxopiperidin-3-yl)-3-methyl-2-oxo-2,3-dihydro-1H-1,3-benzodiazol-4-yl]prop-2-yn-1-yl]oxy)propoxy]propyl]carbamate (900 mg, 1.71 mmol) in THF (20 mL) was added palladium on charcoal (300 mg, 10% w/w) at room temperature under nitrogen atmosphere. The resulting mixture was purged with hydrogen for 3 times and was stirred for 4 h at room temperature under hydrogen atmosphere. The reaction mixture was filtered through a Celite pad. The filtrate was concentrated under reduced pressure to afford tert-butyl N-[3-(3-[3-[1-(2,6-dioxopiperidin-3-yl)-3-methyl-2-oxo-2,3-dihydro-1H-1,3-benzodiazol-4-yl]propoxy]propoxy)propyl]carbamate (800 mg, 88%) as a light brown solid. ¹H NMR (400 MHz, DMSO-d₆) δ 11.10 (br s, 1H), 7.02-6.98 (m, 2H), 6.90-6.83 (m, 1H), 6.76 (t, J=5.7 Hz, 1H), 5.37 (dd, J=12.6, 5.4 Hz, 1H), 3.56 (s, 3H), 3.52-3.33 (m, 6H), 3.01-2.83 (m, 5H), 2.72-2.58 (m, 2H), 2.00 (ddd, J=11.1, 5.9, 3.6 Hz, 1H), 1.89-1.68 (m, 6H), 1.60 (p, J=6.6 Hz, 2H), 1.37 (s, 9H). LC/MS (ESI, m/z): [(M+1)]⁺=533.40

### Step 7: 3-(4-[3-[3-(3-Aminopropoxy)propoxy]propyl]-3-methyl-2-oxo-2,3-dihydro-1H-1,3-benzodiazol-1-yl)piperidine-2,6-dione hydrochloride

To a stirred solution of tert-butyl N-[3-(3-[3-[1-(2,6-dioxopiperidin-3-yl)-3-methyl-2-oxo-2,3-dihydro-1H-1,3-benzodiazol-4-yl]propoxy]propoxy)propyl]carbamate (800 mg, 1.50 mmol) in 1,4-dioxane (10 mL) was added a solution of hydrochloride in dioxane (4 M, 10 mL) dropwise at 0° C. The resulting mixture was stirred for 2 h at room temperature. The resulting mixture was concentrated under reduced pressure to afford 3-(4-[3-[3-(3-aminopropoxy)propoxy]propyl]-3-methyl-2-oxo-2,3-dihydro-1H-1,3-benzodiazol-1-yl)piperidine-2,6-dione hydrochloride (640 mg, 91%) as a white solid. ¹H NMR (400 MHz, DMSO-d₆) δ 11.11 (br s, 1H), 7.83 (br s, 3H), 7.05-6.93 (m, 2H), 6.88 (td, J=6.8, 5.9, 3.1 Hz, 1H), 5.38 (dd, J=12.5, 5.4 Hz, 1H), 3.57 (s, 3H), 3.45-3.31 (m, 8H), 3.04-2.77 (m, 5H), 2.77-2.57 (m, 2H), 1.99 (dd, J=9.8, 4.9 Hz, 1H), 1.90-1.70 (m, 6H). LC/MS (ESI, m/z): [(M+1)]⁺=433.30

### INTERMEDIATE I: 3-(5-(3-(3-(3-aminopropoxy)propoxy)propyl)-3-methyl-2-oxo-2,3-dihydro-1H-benzo[d]imidazol-1-yl)piperidine-2,6-dione hydrochloride

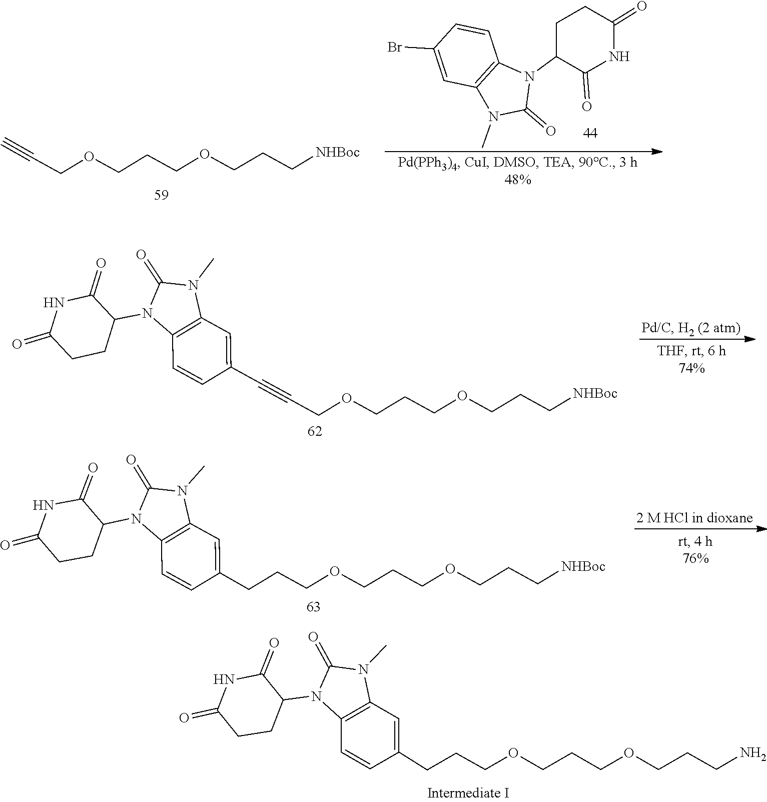 [SMILES: C#CCOCCCOCCCNC(=O)OC(C)(C)C ; Cn1c(=O)n(C2CCC(=O)NC2=O)c2ccc(Br)cc21 ; Cn1c(=O)n(C2CCC(=O)NC2=O)c2ccc(C#CCOCCCOCCCNC(=O)OC(C)(C)C)cc21 ; Cn1c(=O)n(C2CCC(=O)NC2=O)c2ccc(CCCOCCCOCCCNC(=O)OC(C)(C)C)cc21 ; Cn1c(=O)n(C2CCC(=O)NC2=O)c2ccc(CCCOCCCOCCCN)cc21]

### Step 1: tert-Butyl N-[3-[3-([3-[1-(2,6-dioxopiperidin-3-yl)-3-methyl-2-oxo-2,3-dihydro-1H-1,3-benzodiazol-5-yl]prop-2-yn-1-yl]oxy)propoxy]propyl]carbamate

To a stirred solution of 3-(5-bromo-3-methyl-2-oxo-2,3-dihydro-1H-1,3-benzodiazol-1-yl)piperidine-2,6-dione (2.0 g, 5.91 mmol) in DMSO (20 mL) were added tert-butyl N-[3-[3-(prop-2-yn-1-yloxy)propoxy]propyl]carbamate (5.8 g, 21.4 mmol), TEA (10 mL), CuI (0.2 g, 1 mmol) and Pd(PPh₃)₄ (0.7 g, 0.59 mmol) at room temperature under nitrogen atmosphere. The resulting mixture was stirred for 3 h at 90° C. The resulting mixture was cooled to room temperature and concentrated under reduced pressure. The resulting mixture was diluted with 1% aqueous solution of AcOH (100 mL). The resulting mixture was extracted with EtOAc (2×100 mL). The combined organic layers was washed with brine (100 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L AcOH); Eluent B: ACN; Gradient: 45%-55% B in 10 min; Flow rate: 80 mL/min; Detector: 220/254 nm; desired fractions were collected at 51% B and concentrated under reduced pressure to afford tert-butyl N-[3-[3-([3-[1-(2,6-dioxopiperidin-3-yl)-3-methyl-2-oxo-2,3-dihydro-1H-1,3-benzodiazol-5-yl]prop-2-yn-1-yl]oxy)propoxy]propyl]carbamate (1.5 g, 48%) as a yellow solid. ¹H NMR (400 MHz, CDCl₃) δ 8.21 (br s, 1H), 7.22 (dd, J=8.2, 1.5 Hz, 1H), 7.14 (d, J=1.4 Hz, 1H), 6.77 (d, J=8.1 Hz, 1H), 5.22 (dd, J=12.7, 5.3 Hz, 1H), 4.89 (br s, 1H), 4.39 (s, 2H), 3.69 (t, J=6.3 Hz, 2H), 3.55 (t, J=6.3 Hz, 2H), 3.51 (t, J=5.9 Hz, 2H), 3.44 (s, 3H), 3.23 (q, J=6.1 Hz, 2H), 3.03-2.64 (m, 3H), 2.34-2.24 (m, 1H), 1.92 (p, J=6.3 Hz, 2H), 1.76 (p, J=6.3 Hz, 2H), 1.46 (s, 9H). LC/MS (ESI, m/z): [(M+1)]⁺=529.25

### Step 2: tert-Butyl N-[3-(3-[3-[1-(2,6-dioxopiperidin-3-yl)-3-methyl-2-oxo-2,3-dihydro-1H-1,3-benzodiazol-5-yl]propoxy]propoxy)propyl]carbamate

To a solution of tert-butyl N-[3-[3-([3-[1-(2,6-dioxopiperidin-3-yl)-3-methyl-2-oxo-2,3-dihydro-1H-1,3-benzodiazol-5-yl]prop-2-yn-1-yl]oxy)propoxy]propyl]carbamate (200 mg, 0.38 mmol) in THF (10 mL) was added palladium on charcoal (402 mg, 10% w/w) under nitrogen atmosphere. The mixture was purged with hydrogenated for three times and was stirred at room temperature for 6 h under hydrogen atmosphere. The resulting mixture was filtered through a Celite pad and the filtrate was concentrated under reduced pressure to afford tert-butyl N-[3-(3-[3-[1-(2,6-dioxopiperidin-3-yl)-3-methyl-2-oxo-2,3-dihydro-1H-1,3-benzodiazol-5-yl]propoxy]propoxy)propyl]carbamate (150 mg, 74%) as a white solid.

¹H NMR (400 MHz, DMSO-d₆) δ 11.09 (br s, 1H), 7.93 (br s, 1H), 7.06-6.98 (m, 2H), 6.87 (d, J=8.0 Hz, 1H), 5.36 (dt, J=12.6, 6.0 Hz, 1H), 3.57 (s, 5H), 3.47-3.34 (m, 8H), 3.04-2.84 (m, 2H), 2.82-2.66 (m, 1H), 2.66-2.58 (m, 3H), 2.04-1.96 (m, 1H), 1.86-1.68 (m, 4H), 1.65-1.53 (m, 1H), 1.37 (s, 9H). LC/MS (ESI, m/z): [(M+1)]⁺=533.25

### Step 3: 3-(5-[3-[3-(3-Aminopropoxy)propoxy]propyl]-3-methyl-2-oxo-2,3-dihydro-1H-1,3-benzodiazol-1-yl)piperidine-2,6-dione hydrochloride

To a solution of tert-butyl N-[3-(3-[3-[1-(2,6-dioxopiperidin-3-yl)-3-methyl-2-oxo-2,3-dihydro-1H-1,3-benzodiazol-5-yl]propoxy]propoxy)propyl]carbamate (300 mg, 0.56 mmol) in dioxane (10 mL) was added a solution of hydrochloride in 1,4-dioxane (4 M, 10 mL). The resulting solution was stirred for 4 h at room temperature under nitrogen atmosphere. The resulting mixture was concentrated under vacuum to afford 3-(5-[3-[3-(3-aminopropoxy)propoxy]propyl]-3-methyl-2-oxo-2,3-dihydro-1H-1,3-benzodiazol-1-yl)piperidine-2,6-dione hydrochloride (200 mg, 76%) as a yellow solid.

¹H NMR (400 MHz, DMSO-d₆) δ 11.09 (br s, 1H), 7.93 (br s, 3H), 7.06-6.99 (m, 2H), 6.87 (d, J=8.0 Hz, 1H), 5.36 (dd, J=12.7, 5.5 Hz, 1H), 3.58 (s, 3H), 3.48-3.34 (m, 8H), 3.02-2.54 (m, 7H), 2.00 (dd, J=11.4, 6.2 Hz, 1H), 1.87-1.68 (m, 6H)

LC/MS (ESI, m/z): [(M+1)]⁺=433.15

### INTERMEDIATE J: 5-bromo-2-chloro-7-cyclohexyl-7H-pyrrolo[2,3-d]pyrimidine

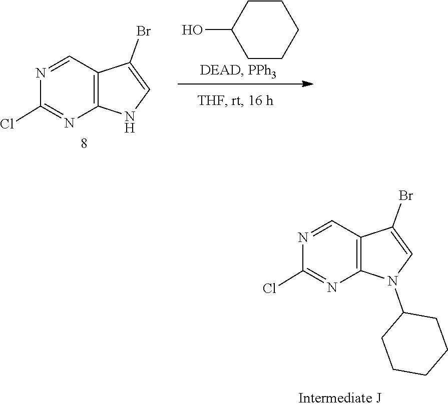 [SMILES: Clc1ncc2c(Br)c[nH]c2n1 ; OC1CCCCC1 ; Clc1ncc2c(Br)cn(C3CCCCC3)c2n1]

To a stirred solution of 5-bromo-2-chloro-7H-pyrrolo[2,3-d]pyrimidine (3.0 g, 12.9 mmol), cyclohexanol (2.59 g, 25.9 mmol) and PPh₃ (8.46 g, 32.3 mmol) in THF (100 mL) was added DEAD (5.62 g, 32.3 mmol) dropwise at 0° C. The resulting solution was stirred for 16 h at room temperature under nitrogen atmosphere. The resulting solution was condensed under reduced pressure. The residue was purified by reversed phase flash chromatography with the following conditions: Column: WelFlash™ C18-I, 20-40 um, 330 g; Eluent A: Water (plus 10 mmol/L TEA); Eluent B: ACN; Gradient: 70%-90% B in 25 min; Flow rate: 80 mL/min; Detector: UV 200/220 nm; desired fractions were collected at 78% B and concentrated under reduced pressure to afford 5-bromo-2-chloro-7-cyclohexyl-7H-pyrrolo[2,3-d]pyrimidine (1.7 g, 42%) as a white solid. ¹H NMR (400 MHz, DMSO-d₆) δ 8.84 (s, 1H), 8.11 (s, 1H), 4.67-4.49 (m, 1H), 1.96-1.64 (m, 7H), 1.47 (q, J=13.5 Hz, 2H), 1.32-1.12 (m, 1H). LC/MS (ESI, m/z): [(M+1)]⁺=313.95, 315.95

### INTERMEDIATE K1: trans-4-[5-bromo-2-[(3,3,3-trifluoropropyl)amino]-7H-pyrrolo[2,3-d]pyrimidin-7-yl]cyclohexan-1-ol

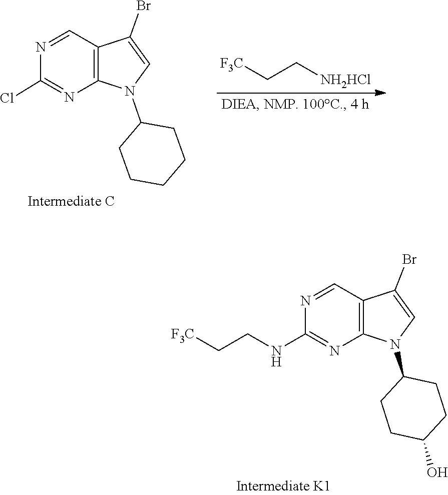

To a solution of trans-4-[5-bromo-2-chloro-7H-pyrrolo[2,3-d]pyrimidin-7-yl]cyclohexan-1-ol (5.0 g, 15.1 mmol) in NMP (25.0 mL) were added DIEA (11.7 g, 90.7 mmol) and 3,3,3-trifluoropropan-1-amine hydrochloride (4.52 g, 30.3 mmol) at room temperature under nitrogen atmosphere. The resulting solution was stirred for 20 h at 110° C. in a sealed tube. After cooling down to room temperature, the resulting solution was purified by reversed phase flash chromatography with the following conditions: Column: WelFlash™ C18-I, 20-40 um, 330 g; Eluent A: Water (plus 10 mmol/L FA); Eluent B: ACN; Gradient: 40%-60% B in 25 min; Flow rate: 80 mL/min; Detector: UV 220/254 nm; desired fractions were collected at 52% B and concentrated under reduced pressure to afford trans-4-[5-bromo-2-[(3,3,3-trifluoropropyl)amino]-7H-pyrrolo[2,3-d]pyrimidin-7-yl]cyclohexan-1-ol (4.8 g, 78%) as a white solid. ¹H NMR (400 MHz, DMSO-d₆) δ 8.41 (s, 1H), 7.44 (s, 1H), 7.21 (t, J=5.8 Hz, 1H), 4.70 (d, J=4.5 Hz, 1H), 4.39 (dq, J=11.4, 6.7, 5.6 Hz, 1H), 3.60-3.53 (m, 2H), 3.49 (dd, J=10.7, 4.4 Hz, 1H), 2.58 (dt, J=11.6, 7.2 Hz, 2H), 1.85-1.96 (m, 6H), 1.39-1.27 (m, 2H). LC/MS (ESI, m/z): [(M+1)]⁺=407.05, 409.05.

The following intermediates in Table 2 were synthesized according to the above procedure

TABLE 2

| Inter | | | MS: | |
|---|---|---|---|---|
| mediate | Structure | Name | [(M + 1)]⁺ | ¹H NMR |
| K2 | 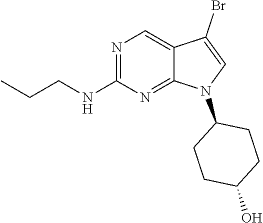 [SMILES: CCCNc1ncc2c(Br)cn([C@H]3CC[C@H](O)CC3)c2n1] | trans-4-[5-bromo- 2-(propylamino)- 7H-pyrrolo[2,3- d]pyrimidin-7- yl]cyclohexan-1- ol | 353.10, 355.10. | (400 MHz, CD₃OD) δ 8.28 (s, 1H), 7.14 (d, J = 2.1 Hz, 1H), 4.54-4.41 (m, 1H), 3.67 (m, 1H), 3.37 (t, J = 7.1 Hz, 2H), 2.13-2.04 (m, 2H), 2.03-1.85 (m, 4H), 1.69-1.60 (m, 2H), 1.52- 1.47 (m, 2H), 0.99 (t, J = 7.5 Hz, 3H) |
| K3 | 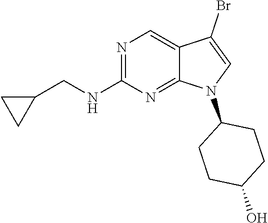 [SMILES: O[C@H]1CC[C@H](n2cc(Br)c3cnc(NCC4CC4)nc32)CC1] | trans-4-[5-bromo- 2-[(cyclopropyl- methyl)amino]- 7H-pyrrolo[2,3- d]pyrimidin-7- yl]cyclohexan- 1-ol | 365.05, 367.05. | (400 MHz, CD₃OD) δ 8.31 (m, 1H), 7.18 (m, 1H), 4.55-4.43 (m, 1H), 3.73- 3.63 (m, 1H), 3.37-3.26 (m, 2H), 2.10 (m, 2H), 2.08-1.93 (m, 4H), 1.58- 1.47 (m, 2H), 1.25-1.01 (m, 1H), 0.59-0.51 (m, 1H), 0.51 (d, J = 6.3 Hz, 1H), 0.39-0.25 (m, 2H) |
| K4 | 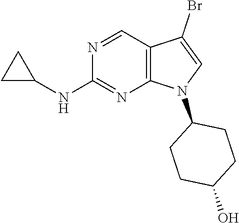 [SMILES: O[C@H]1CC[C@H](n2cc(Br)c3cnc(NC4CC4)nc32)CC1] | trans-4-[5-bromo- 2-(cyclopropyl- amino)-7H- pyrrolo[2,3-d]- pyrimidin-7-yl]- cyclohexan-1-ol | 351.05, 353.05. | (400 MHz, CD₃OD) δ 8.51 (s, 1H), 7.74 (s, 1H), 3.82- 3.64 (m, 2H), 3.25 (q, J = 7.4 Hz, 1H), 2.18-2.06 (m, 2H), 2.07-1.91 (m, 4H), 1.56-1.43 (m, 2H), 1.06- 0.98 (m, 2H), 0.83-0.69 (m, 2H) |
| K5 | 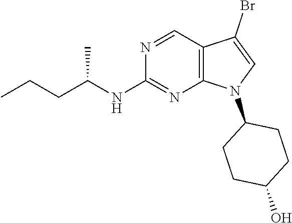 [SMILES: CCC[C@H](C)Nc1ncc2c(Br)cn([C@H]3CC[C@H](O)CC3)c2n1] | trans-4-[5-bromo- 2-[(2S)-pentan-2- ylamino]pyrrolo- [2,3-d]pyrimidin- 7-yl]cyclohexan- 1-ol | 381.05, 383.05. | (400 MHz, CD₃OD) δ 8.29 (s, 1H), 7.16 (s, 1H), 4.55- 4.44 (m, 1H), 4.10 (h, J = 6.5 Hz, 1H), 3.69 (m, 1H), 2.13-2.08 (m, 2H), 2.02- 1.97 (m, 3H), 1.96-1.91 (m, 1H), 1.67-1.60 (m, 1H), 1.54-1.43 (m, 5H), 1.24 (d, J = 6.5 Hz, 3H), 0.98 (t, J = 7.1 Hz, 3H) |
| K6 | 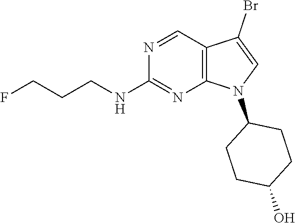 [SMILES: O[C@H]1CC[C@H](n2cc(Br)c3cnc(NCCCF)nc32)CC1] | trans-4-[5-bromo- 2-[(3-fluoro- propyl)amino]- 7H-pyrrolo[2,3- d]pyrimidin-7- yl]cyclohexan-1- ol | 371.15, 373.15. | (400 MHz, DMSO-d₆) δ 8.36 (s, 1H), 7.39 (s, 1H), 7.16-7.06 (m, 1H), 4.72- 4.55 (m, 2H), 4.51-4.32 (m, 2H), 3.58-3.42 (m, 2H), 1.98-1.79 (m, 8H), 1.38-1.26 (m, 2H) |
| K7 | 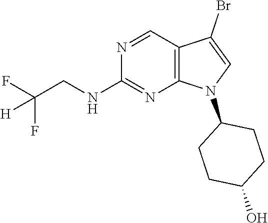 [SMILES: O[C@H]1CC[C@H](n2cc(Br)c3cnc(NCC(F)F)nc32)CC1] | trans-4-[5-bromo- 2-[(2,2-difluoro- ethyl)amino]-7H- pyrrolo[2,3-d]- pyrimidin-7-yl]- cyclohexan-1-ol | 375.20, 377.20. | (400 MHz, CD₃OD) δ 8.56 (d, J = 1.7 Hz, 1H), 7.71- 7.62 (m, 1H), 6.13 (tt, J = 55.9, 3.8 Hz, 1H), 4.62- 4.55 (m, 1H), 3.99-3.85 (m, 2H), 3.75-3.70 (m, 1H), 2.04-1.94 (m, 4H), 1.40-1.37 (m, 4H) |
| K8 | 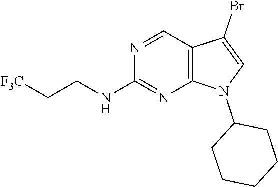 [SMILES: FC(F)(F)CCNc1ncc2c(Br)cn(C3CCCCC3)c2n1] | 5-bromo-7-cyclo- hexyl-N-(3,3,3- trifluoropropyl)- 7H-pyrrolo[2,3- d]pyrimidin-2- amine | 391.05, 393.05. | (400 MHz, DMSO-d₆) δ 8.41 (s, 1H), 7.44 (s, 1H), 7.18 (s, 1H), 4.40 (d, J = 11.9 Hz, 1H), 3.54 (q, J = 6.7 Hz, 2H), 2.59-2.45 (m, 2H), 1.89-1.75 (m, 6H), 1.69 (d, J = 13.1 Hz, 1H), 1.38 (d, J = 13.0 Hz, 2H), 1.23 (t, J = 12.9 Hz, 1H) |
| K9 | 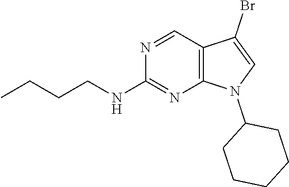 [SMILES: CCCCNc1ncc2c(Br)cn(C3CCCCC3)c2n1] | 5-bromo-N-butyl- 7-cyclohexyl-7H- pyrrolo-[2,3-d]- pyrimidin-2-amine | 351.05, 353.05. | (400 MHz, DMSO-d₆) δ 8.35 (s, 1H), 7.39 (s, 1H), 6.99 (d, J = 5.9 Hz, 1H), 4.45-4.32 (m, 1H), 3.30 (d, J = 6.7 Hz, 2H), 1.88-1.79 (m, 6H), 1.75-1.65 (m, 1H), 1.59-1.42 (m, 2H), 1.40- 1.29 (m, 4H), 1.25-1.18 (m, 1H), 0.91 (t, J = 7.3 Hz, 3H) |

### INTERMEDIATE K10: trans-4-[5-bromo-2-[(3,3,3-trifluoropropyl)amino]pyrrolo[2,3-d]pyrimidin-7-yl]-1-methylcyclohexan-1-ol and

### INTERMEDIATE K11: cis-4-[5-bromo-2-[(3,3,3-trifluoropropyl)amino]pyrrolo[2,3-d]pyrimidin-7-yl]-1-methylcyclohexan-1-ol

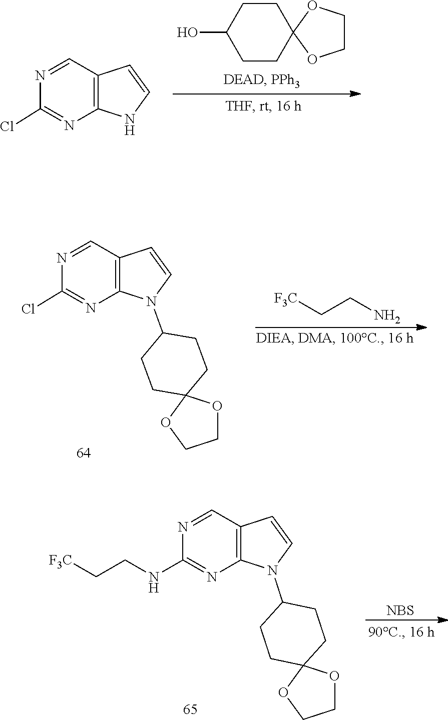 [SMILES: Clc1ncc2cc[nH]c2n1 ; OC1CCC2(CC1)OCCO2 ; Clc1ncc2ccn(C3CCC4(CC3)OCCO4)c2n1 ; NCCC(F)(F)F ; FC(F)(F)CCNc1ncc2ccn(C3CCC4(CC3)OCCO4)c2n1 ; "NBS"]

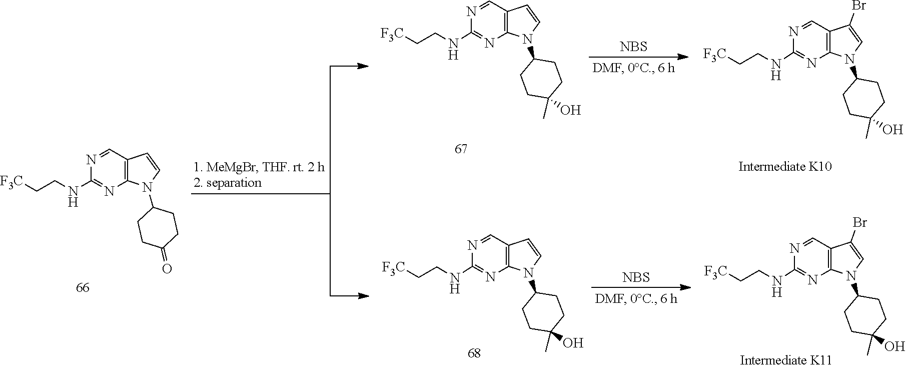

### Step 1: 2-chloro-7-[1,4-dioxaspiro[4.5]decan-8-yl]-7H-pyrrolo[2,3-d]pyrimidine

To a stirred solution of 2-chloro-7H-pyrrolo[2,3-d]pyrimidine (50.0 g, 326 mmol), 1,4-dioxaspiro[4.5]decan-8-ol (103 g, 651 mmol) and PPh₃ (214 g, 814 mmol) in THF (800 mL) was added DEAD (142 g, 814 mmol) dropwise at 0° C. under nitrogen atmosphere. The solution was stirred for 16 h at room temperature. The solution was condensed under reduced pressure. The crude product was re-crystallized from MeOH (300 mL) to afford 2-chloro-7-[1,4-dioxaspiro[4.5]decan-8-yl]-7H-pyrrolo[2,3-d]pyrimidine (68.0 g, 71%) as a white solid. ¹H NMR (400 MHz, DMSO-d₆) δ 8.90 (s, 1H), 7.79 (d, J=3.7 Hz, 1H), 6.68 (d, J=3.6 Hz, 1H), 4.77-4.65 (m, 1H), 3.97-3.83 (m, 4H), 2.15-2.00 (m, 2H), 1.96-1.87 (m, 2H), 1.85-1.73 (m, 4H). LC/MS (ESI, m/z): [(M+1)]⁺=294.05

### Step 2: 7-[1,4-dioxaspiro[4.5]decan-8-yl]-N-(3,3,3-trifluoropropyl)-7H-pyrrolo[2,3-d]pyrimidin-2-amine

To a stirred mixture of 2-chloro-7-[1,4-dioxaspiro[4.5]decan-8-yl]-7H-pyrrolo[2,3-d]pyrimidine (2.00 g, 6.81 mmol) and 3,3,3-trifluoropropan-1-amine (1.54 g, 13.6 mmol) in 1,4-dioxane (30.0 mL) were added Cs₂CO₃ (11.1 g, 34.1 mmol), BINAP (0.85 g, 1.36 mmol) and Pd₂(dba)₃ (0.62 g, 0.68 mmol) at room temperature. The resulting mixture was purged with nitrogen three times and stirred for 16 h at 100° C. under nitrogen atmosphere. The mixture was cooled down to room temperature and concentrated under reduced pressure. The residue was purified by reversed phase flash chromatography with the following conditions: Column: WelFlash™ C18-I, 20-40 um, 330 g; Eluent A: Water (plus 10 mmol/L TEA); Eluent B: ACN; Gradient: 40%-60% B in 20 min; Flow rate: 80 mL/min; Detector: UV 220/254 nm; desired fractions were collected at 53% B and concentrated under reduced pressure to afford 7-[1,4-dioxaspiro[4.5] decan-8-yl]-N-(3,3,3-trifluoropropyl)-7H-pyrrolo[2,3-d]pyrimidin-2-amine (1.37 g, 54%) as a white solid. ¹H NMR (400 MHz, DMSO-d₆) δ 8.51 (s, 1H), 7.19 (d, J=3.7 Hz, 1H), 6.92 (t, J=5.8 Hz, 1H), 6.32 (d, J=3.7 Hz, 1H), 4.48 (tt, J=12.1, 3.9 Hz, 1H), 3.96-3.87 (m, 4H), 3.54 (dt, J=7.6, 6.0 Hz, 2H), 2.65-2.53 (m, 2H), 2.13-1.99 (m, 2H), 1.93-1.78 (m, 4H), 1.68 (td, J=13.5, 4.1 Hz, 2H). LC/MS (ESI, m/z): [(M+1)]⁺=371.15

### Step 3: 4-[2-[(3,3,3-trifluoropropyl)amino]-7H-pyrrolo[2,3-d]pyrimidin-7-yl]cyclohexan-1-one

To a stirred solution of 7-[1,4-dioxaspiro[4.5]decan-8-yl]-N-(3,3,3-trifluoropropyl)-7H-pyrrolo[2,3-d]pyrimidin-2-amine (866 mg, 2.34 mmol) in H₂O (15.0 mL) and acetonitrile (15.0 mL) was added TsOH (423 mg, 2.45 mmol) at room temperature. The resulting mixture was stirred for 16 h at 90° C. under nitrogen atmosphere. The mixture was cooled down to room temperature. The resulting mixture was concentrated under vacuum. The residue was purified by reversed phase flash chromatography with the following conditions: Column: WelFlash™ C18-I, 20-40 um, 330 g; Eluent A: Water (plus 10 mmol/L TEA); Eluent B: ACN; Gradient: 40%-60% B in 20 min; Flow rate: 80 mL/min; Detector: UV 220/254 nm; desired fractions were collected at 50% B and concentrated under reduced pressure to afford 4-[2-[(3,3,3-trifluoropropyl)amino]-7H-pyrrolo[2,3-d]pyrimidin-7-yl]cyclohexan-1-one (640 mg, 84%) as a white solid. ¹H NMR (400 MHz, DMSO-d₆) δ 8.54 (s, 1H), 7.25 (d, J=3.7 Hz, 1H), 6.93 (t, J=5.9 Hz, 1H), 6.36 (d, J=3.7 Hz, 1H), 4.94 (tt, J=11.8, 3.9 Hz, 1H), 3.63-3.50 (m, 2H), 2.71-2.54 (m, 4H), 2.39-2.26 (m, 4H), 2.21-2.11 (m, 2H). LC/MS (ESI, m/z): [(M+1)]⁺=327.10

### Step 4: Trans-1-methyl-4-[2-[(3,3,3-trifluoropropyl)amino]-7H-pyrrolo[2,3-d]pyrimidin-7-yl]cyclohexan-1-ol and cis-1-methyl-4-[2-[(3,3,3-trifluoropropyl)amino]-7H-pyrrolo[2,3-d]pyrimidin-7-yl]cyclohexan-1-ol

To a stirred solution of 4-[2-[(3,3,3-trifluoropropyl)amino]-7H-pyrrolo[2,3-d]pyrimidin-7-yl]cyclohexan-1-one (800 mg, 2.45 mmol) in THF (8.00 mL) were added LiCl (208 mg, 4.90 mmol) and MeMgBr (25 mL, 12.5 mmol, 0.5 M in THF) dropwise at 0° C. The resulting mixture was stirred for 2 h at room temperature under nitrogen atmosphere. The reaction was quenched by sat. NH₄Cl (20.0 mL) and extracted by EtOAc (3×20.0 mL). The combined organic layer was washed with brine (20.0 mL), dried with anhydrous Na₂SO₄ and filtered. The filtrate was concentrated under reduced pressure. The residue was purified by Prep-HPLC with the following conditions (Column: XBridge Prep OBD C₁₈ Column 30×150 mm, 5 um; Mobile Phase A: water (plus 10 mmol/L NH₄HCO₃), Mobile Phase B: ACN; Flow rate: 60 mL/min; Gradient: 25% B to 45% B in 11 min; Detector: UV 220/254 nm) to afford trans-1-methyl-4-[2-[(3,3,3-trifluoropropyl)amino]-7H-pyrrolo[2,3-d]pyrimidin-7-yl]cyclohexan-1-ol (67, Rt: 8.25 min) (253 mg, 30%) as a white solid. ¹H NMR (400 MHz, CD₃OD) δ 8.44 (s, 1H), 7.15 (d, J=3.7 Hz, 1H), 6.38 (d, J=3.7 Hz, 1H), 4.50 (tt, J=12.0, 4.2 Hz, 1H), 3.70 (dd, J=8.1, 6.4 Hz, 2H), 2.56 (qt, J=11.2, 7.3 Hz, 2H), 2.18-2.03 (m, 2H), 1.97 (dd, J=13.6, 4.0 Hz, 2H), 1.88-1.68 (m, 4H), 1.41 (s, 3H). LC/MS (ESI, m/z): [(M+1)]⁺=343.20 and cis-1-methyl-4-[2-[(3,3,3-trifluoropropyl)amino]-7H-pyrrolo[2,3-d]pyrimidin-7-yl]cyclohexan-1-ol (68, Rt: 9.70 min) (465 mg, 55%) as a white solid. ¹H NMR (400 MHz, CD₃OD) δ 8.44 (s, 1H), 7.16 (dd, J=3.8, 0.9 Hz, 1H), 6.39 (dd, J=3.7, 0.9 Hz, 1H), 4.54 (tt, J=12.3, 3.8 Hz, 1H), 3.70 (t, J=7.1 Hz, 2H), 2.57 (qt, J=11.2, 7.0 Hz, 2H), 2.25-2.16 (m, 2H), 1.89-1.75 (m, 4H), 1.65 (td, J=14.5, 13.8, 4.3 Hz, 2H), 1.29 (s, 3H). LC/MS (ESI, m/z): [(M+1)]⁺=343.20

### Step 5: trans-4-[5-bromo-2-[(3,3,3-trifluoropropyl)amino]pyrrolo[2,3-d]pyrimidin-7-yl]-1-methylcyclohexan-1-ol and cis-4-[5-bromo-2-[(3,3,3-trifluoropropyl)amino]pyrrolo[2,3-d]pyrimidin-7-yl]-1-methylcyclohexan-1-ol

To a stirred solution of trans-1-methyl-4-[2-[(3,3,3-trifluoropropyl)amino]pyrrolo[2,3-d]pyrimidin-7-yl]cyclohexan-1-ol (100 mg, 0.29 mmol) in DMF (7.00 mL) was added a solution of NBS (57.2 mg, 0.32 mmol) in DMF (2.00 mL) dropwise at 0° C. under nitrogen atmosphere. After stirring for additional 6 h, the resulting solution was purified by reversed phase flash chromatography with the following conditions: Column: WelFlash™ C18-I, 20-40 um, 120 g; Eluent A: water (plus 10 mmol/L NH₄HCO₃); Eluent B: ACN; Gradient: 40%-60% B in 20 min; Flow rate: 60 mL/min; Detector: UV 220/254 nm; desired fractions were collected at 48% B and concentrated under reduced pressure to afford trans-4-[5-bromo-2-[(3,3,3-trifluoropropyl)amino]pyrrolo[2,3-d]pyrimidin-7-yl]-1-methylcyclohexan-1-ol (70.0 mg, 57%) as a pink solid. ¹H NMR (400 MHz, DMSO-d₆) δ 8.41 (s, 1H), 7.52 (s, 1H), 7.19 (t, J=5.8 Hz, 1H), 4.55-4.31 (m, 2H), 3.64-3.42 (m, 2H), 2.58 (tt, J=8.8, 5.8 Hz, 2H), 1.98 (q, J=12.8 Hz, 2H), 1.87-1.74 (m, 2H), 1.72-1.62 (m, 2H), 1.59-1.51 (m, 2H), 1.27 (s, 3H). LC/MS (ESI, m/z): [(M+1)]⁺=421.15, 423.15

The following intermediates in Table 3 were synthesized according to the above procedure.

TABLE 3

| Inter- | | | MS: | |
|---|---|---|---|---|
| mediate | Structure | Name | [(M + 1)]⁺ | ¹H NMR |
| K11 | 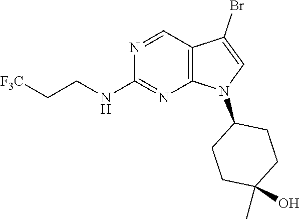 [SMILES: C[C@]1(O)CC[C@@H](n2cc(Br)c3cnc(NCCC(F)(F)F)nc32)CC1] | cis-4-[5-bromo-2- [(3,3,3-trifluoro- propyl)amino]- pyrrolo[2,3-d]- pyrimidin-7-yl]- 1-methylcyclo- hexan-1-ol | 421.15, 423.15 | (400 MHz, DMSO-d₆) δ 8.41 (s, 1H), 7.40 (s, 1H), 7.15 (s, 1H), 4.39 (t, J = 12.1 Hz, 1H), 3.55 (q, J = 6.7 Hz, 2H), 2.57 (dt, J = 11.4, 6.9 Hz, 2H), 2.17- 2.06 (m, 2H), 1.75-1.59 (m, 4H), 1.53-1.41 (m, 2H), 1.17 (s, 3H) |

### INTERMEDIATE K12: trans-4-[3-bromo-6-[(3,3,3-trifluoropropyl)amino]-1H-pyrazolo[3,4-d]pyrimidin-1-yl]cyclohexan-1-ol

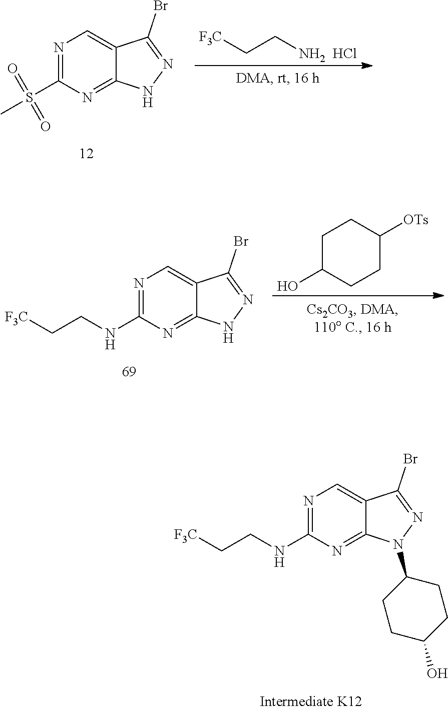 [SMILES: CS(=O)(=O)c1ncc2c(Br)n[nH]c2n1 ; NCCC(F)(F)F ; Cl ; FC(F)(F)CCNc1ncc2c(Br)n[nH]c2n1 ; Cc1ccc(S(=O)(=O)OC2CCC(O)CC2)cc1 ; O[C@H]1CC[C@H](n2nc(Br)c3cnc(NCCC(F)(F)F)nc32)CC1]

### Step 1: 3-bromo-N-(3,3,3-trifluoropropyl)-1H-pyrazolo[3,4-d]pyrimidin-6-amine

To a stirred mixture of 3-bromo-6-methanesulfonyl-1H-pyrazolo[3,4-d]pyrimidine (2.30 g, 8.30 mmol) and TEA (12.6 g, 125 mmol) in DMA (20.0 mL) was added 3,3,3-trifluoropropan-1-amine hydrochloride (6.21 g, 41.5 mmol) at room temperature under nitrogen atmosphere. After stirring for additional 16 h, the resulting mixture was diluted with water (50.0 mL). The precipitated solid was collected by filtration and washed with water (2×10.0 mL) to give 3-bromo-N-(3,3,3-trifluoropropyl)-1H-pyrazolo[3,4-d]pyrimidin-6-amine (2.0 g, 78%) as light yellow solid. ¹H NMR (400 MHz, DMSO-d₆) δ 13.31 (s, 1H), 8.70 (s, 1H), 7.86 (s, 1H), 3.55 (s, 2H), 2.58 (td, J=11.1, 5.0 Hz, 2H). LC/MS (ESI, m/z): [(M+1)]⁺=310.00, 312.00

### Step 2: trans-4-[3-bromo-6-[(3,3,3-trifluoropropyl)amino]-1H-pyrazolo[3,4-d]pyrimidin-1-yl]cyclohexan-1-ol

To a stirred mixture of 3-bromo-N-(3,3,3-trifluoropropyl)-1H-pyrazolo[3,4-d]pyrimidin-6-amine (2.00 g, 6.45 mmol) and 4-hydroxycyclohexyl 4-methylbenzene-1-sulfonate (4.36 g, 16.2 mmol) in DMA (20.0 mL) was added Cs₂CO₃ (6.30 g, 19.4 mmol) at room temperature under nitrogen atmosphere. The resulting mixture was stirred at for 16 h 110° C. Upon completion, the mixture was cooled down to room temperature. The resulting mixture was diluted with EtOAc (50.0 mL) and water (50.0 mL). The resulting mixture was extracted with EtOAc (2×30.0 mL). The combined organic layer was dried over anhydrous Na₂SO₄. After filtration, the filtrate was concentrated under reduced pressure. The residue was purified by silica gel column chromatography, eluted with 25% ethyl acetate in petroleum ether to afford trans-4-[3-bromo-6-[(3,3,3-trifluoropropyl)amino]-1H-pyrazolo[3,4-d]pyrimidin-1-yl]cyclohexan-1-ol (500 mg, 19%) as an off-white solid. ¹H NMR (400 MHz, DMSO-d₆) δ 8.79-8.65 (m, 1H), 7.89-7.75 (m, 1H), 4.69 (d, J=4.3 Hz, 1H), 4.44 (s, 1H), 3.59-3.45 (m, 3H), 2.60 (s, 2H), 2.05-1.90 (m, 6H), 1.34 (d, J=12.3 Hz, 2H). LC/MS (ESI, m/z): [(M+1)]⁺=408.00, 410.00

### INTERMEDIATE L1: trans-4-[7-[4-hydroxycyclohexyl]-2-[(3,3,3-trifluoropropyl)amino]pyrrolo[2,3-d]pyrimidin-5-yl]benzaldehyde

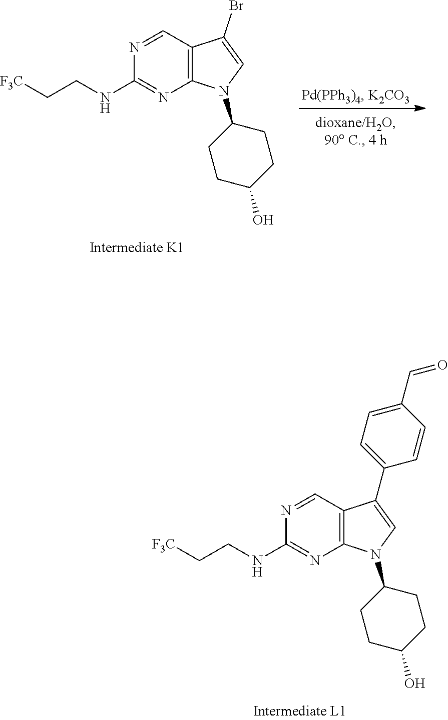 [SMILES: O[C@H]1CC[C@H](n2cc(Br)c3cnc(NCCC(F)(F)F)nc32)CC1 ; O=Cc1ccc(-c2cn([C@H]3CC[C@H](O)CC3)c3nc(NCCC(F)(F)F)ncc23)cc1]

A solution of trans-4-[5-bromo-2-[(3,3,3-trifluoropropyl)amino]pyrrolo[2,3-d]pyrimidin-7-yl]cyclohexan-1-ol (2.00 g, 4.91 mmol), Pd(PPh₃)₄ (567 mg, 0.049 mmol), K₂CO₃ (2.04 g, 14.7 mmol,) and 4-(4,4,5,5-tetramethyl-1,3,2-dioxaborolan-2-yl)benzaldehyde (1.71 g, 7.37 mmol) in dioxane (15.0 mL) and H₂O (5.00 mL) were stirred for 4 h at 90° C. under nitrogen atmosphere. The mixture was cooled down to room temperature. The resulting solution was purified by reversed phase flash chromatography with the following conditions: Column: WelFlash™ C18-I, 20-40 mm, 330 g; Eluent A: water (plus 10 mmol/L NH₄HCO₃); Eluent B: ACN; Gradient: 45%-65% B in 15 min; Flow rate: 80 mL/min; Detector: UV 220/254 nm; desired fractions were collected at 60% B and concentrated under reduced pressure to afford trans-4-[7-[4-hydroxycyclohexyl]-2-[(3,3,3-trifluoropropyl)amino]pyrrolo[2,3-d]pyrimidin-5-yl]benzaldehyde (1.50 g, 71%) as a yellow solid. ¹H NMR (400 MHz, CD₃OD) δ 9.96 (d, J=2.2 Hz, 1H), 8.87 (d, J=6.1 Hz, 1H), 7.95 (dd, J=8.4, 2.8 Hz, 2H), 7.90-7.78 (m, 2H), 7.67 (d, J=9.9 Hz, 1H), 4.59-4.51 (m, 1H), 3.80-3.69 (m, 3H), 3.29-3.18 (m, 2H), 2.19-2.03 (m, 6H), 1.63-1.51 (m, 2H). LC/MS (ESI, m/z): [(M+1)]⁺=433.20

The following intermediates in Table 4 were synthesized according to the above procedure.

TABLE 4

| Inter | | | MS: | |
|---|---|---|---|---|
| mediate | Structure | Name | [(M + 1)]⁺ | ¹H NMR |
| L2 | 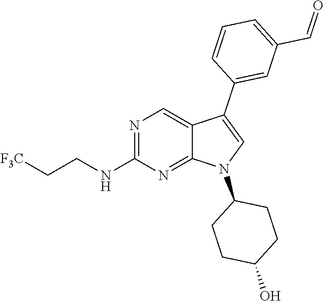 [SMILES: O=Cc1cccc(-c2cn([C@H]3CC[C@H](O)CC3)c3nc(NCCC(F)(F)F)ncc23)c1] | trans-3-[7-[4- hydroxycyclo- hexyl]-2-[(3,3,3- trifluoropropyl)- amino]pyrrolo- [2,3-d]pyrimidin- 5-yl]benzalde- hyde | 433.25 | (400 MHz, DMSO-d₆) δ 10.10 (s, 1H), 8.99 (s, 1H), 8.24 (t, J = 1.7 Hz, 1H), 8.05 (dt, J = 7.9, 1.6 Hz, 1H), 7.86 (s, 1H), 7.79- 7.67 (m, 1H), 7.63 (s, 1H), 7.13 (t, J = 5.9 Hz, 1H), 4.71 (d, J = 4.3 Hz, 1H), 4.50-4.41 (m, 1H), 3.59 (q, J = 6.6 Hz, 2H), 2.61 (dt, J = 11.5, 7.1 Hz, 2H), 2.03-1.89 (m, 6H), 1.39- 1.30 (m, 2H) |
| L3 | 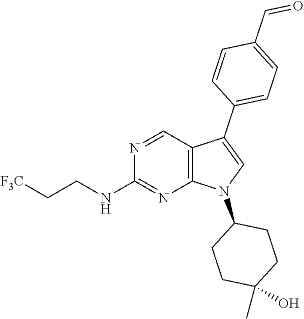 [SMILES: C[C@]1(O)CC[C@H](n2cc(-c3ccc(C=O)cc3)c3cnc(NCCC(F)(F)F)nc32)CC1] | trans-4-[7-[4- hydroxy-4-meth- ylcyclohexyl]-2- [(3,3,3-trifluoro- propyl)amino]- pyrrolo[2,3-d]- pyrimidin-5-yl]- benzaldehyde | 447.25 | (400 MHz, DMSO-d₆) δ 9.98 (s, 1H), 9.03 (s, 1H), 8.06 (s, 1H), 7.99-7.90 (m, 4H), 7.16 (t, J = 5.8 Hz, 1H), 4.48 (s, 1H), 3.93 (d, J = 5.7 Hz, 1H), 3.59 (q, J = 6.8 Hz, 2H), 2.60 (dt, J = 11.6, 7.2 Hz, 2H), 2.11- 1.99 (m, 2H), 1.85 (d, J = 12.5 Hz, 2H), 1.54 (s, 2H), 1.24 (d, J = 4.1 Hz, 2H), 0.94 (s, 3H) |
| L4 | 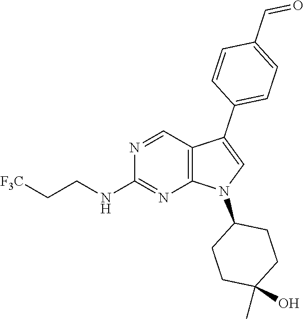 [SMILES: C[C@]1(O)CC[C@@H](n2cc(-c3ccc(C=O)cc3)c3cnc(NCCC(F)(F)F)nc32)CC1] | cis-4-[7-[4- hydroxy-4-meth- ylcyclohexyl]-2- [(3,3,3-trifluoro- propyl)amino]- pyrrolo[2,3-d]- pyrimidin-5-yl]- benzaldehyde | 447.25 | (400 MHz, CD₃OD) δ 9.98 (s, 1H), 8.88 (s, 1H), 7.97 (d, J = 8.3 Hz, 2H), 7.89 (d, J = 8.3 Hz, 2H), 7.72 (d, J = 3.3 Hz, 1H), 4.62 (t, J = 12.4 Hz, 1H), 3.74 (t, J = 7.1 Hz, 2H), 2.63-2.51 (m, 2H), 2.36-2.22 (m, 2H), 1.93-1.64 (m, 6H), 1.31 (s, 3H) |

### INTERMEDIATE L5: trans-7-[4-hydroxycyclohexyl]-2-[(3,3,3-trifluoropropyl)amino]pyrrolo[2,3-d]pyrimidine-5-carbaldehyde

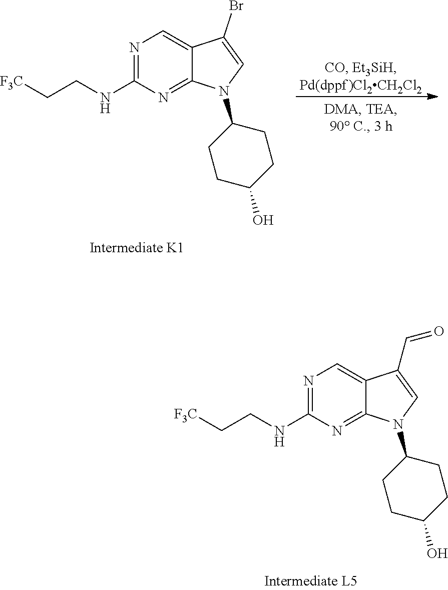 [SMILES: O[C@H]1CC[C@H](n2cc(Br)c3cnc(NCCC(F)(F)F)nc32)CC1 ; O=Cc1cn([C@H]2CC[C@H](O)CC2)c2nc(NCCC(F)(F)F)ncc12]

To a stirred solution of trans-4-[5-bromo-2-[(3,3,3-trifluoropropyl)amino]pyrrolo[2,3-d]pyrimidin-7-yl]cyclohexan-1-ol (250 mg, 0.61 mmol) and Et₃SiH (143 mg, 1.23 mmol) in DMA (12.0 mL) were added TEA (186 mg, 1.84 mmol) and Pd(dppf)Cl₂.CH₂Cl₂ (50.0 mg, 0.06 mmol) at room temperature. The resulting mixture was purged with CO three times and stirred for 3 h at 90° C. under CO atmosphere. After cooling down to room temperature, the mixture was purified by reversed phase flash chromatography with the following conditions: Column: WelFlash™ C18-I, 20-40 um, 330 g; Eluent A: water (plus 10 mmol/L FA); Eluent B: ACN; Gradient: 25%-45% B in 20 min; Flow rate: 80 mL/min; Detector: UV 220/254 nm; desired fractions were collected at 38% B and concentrated under reduced pressure to afford trans-7-[4-hydroxycyclohexyl]-2-[(3,3,3-trifluoropropyl)amino]pyrrolo[2,3-d]pyrimidine-5-carbaldehyde (190 mg, 87%) as a white solid. ¹H NMR (400 MHz, DMSO-d₆) δ 9.77 (s, 1H), 8.87 (s, 1H), 8.27 (s, 1H), 7.35 (d, J=6.5 Hz, 1H), 4.72 (d, J=4.4 Hz, 1H), 4.43 (p, J=6.1, 5.2 Hz, 1H), 3.63-3.50 (m, 3H), 2.64-2.53 (m, 2H), 2.06-1.91 (m, 6H), 1.39-1.32 (m, 2H). LC/MS (ESI, m/z): [(M+1)]⁺=357.15

### INTERMEDIATE L6: trans-4-[7-[trans-4-[(tert-butyldimethylsilyl)oxy]cyclohexyl]-2-[(3,3,3-trifluoropropyl)amino]pyrrolo[2,3-d]pyrimidin-5-yl]cyclohexane-1-carbaldehyde and Intermediate L7: cis-4-[7-[trans-4-[(tert-butyldimethylsilyl)oxy]cyclohexyl]-2-[(3,3,3-trifluoropropyl)amino]pyrrolo[2,3-d]pyrimidin-5-yl]cyclohexane-1-carbaldehyde

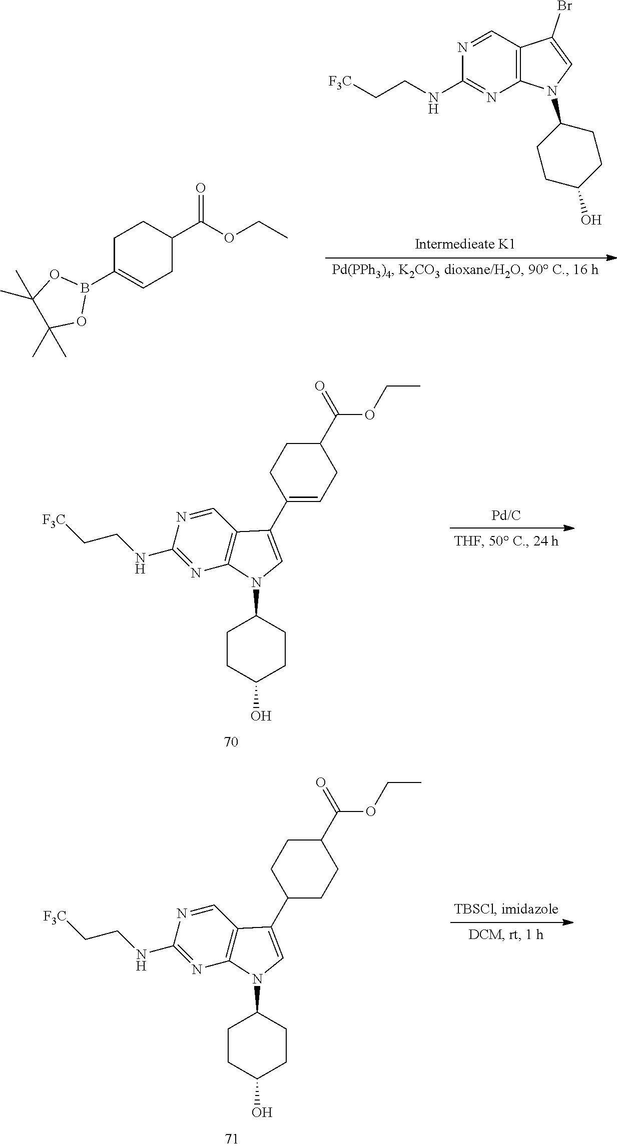 [SMILES: CCOC(=O)C1CC=C(B2OC(C)(C)C(C)(C)O2)CC1 ; O[C@H]1CC[C@H](n2cc(Br)c3cnc(NCCC(F)(F)F)nc32)CC1 ; CCOC(=O)C1CC=C(c2cn([C@H]3CC[C@H](O)CC3)c3nc(NCCC(F)(F)F)ncc23)CC1 ; CCOC(=O)C1CCC(c2cn([C@H]3CC[C@H](O)CC3)c3nc(NCCC(F)(F)F)ncc23)CC1]

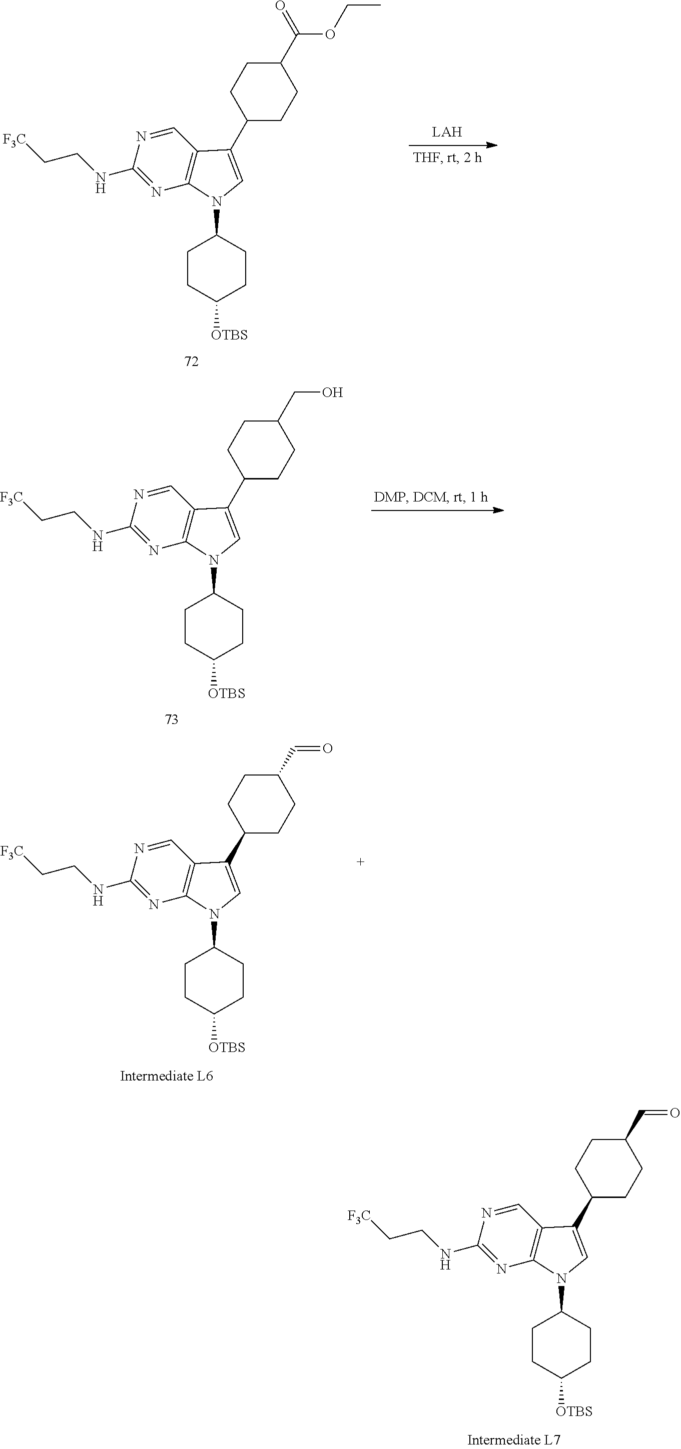 [SMILES: CCOC(=O)C1CCC(c2cn([C@H]3CC[C@H](O[Si](C)(C)C(C)(C)C)CC3)c3nc(NCCC(F)(F)F)ncc23)CC1 ; CC(C)(C)[Si](C)(C)O[C@H]1CC[C@H](n2cc(C3CCC(CO)CC3)c3cnc(NCCC(F)(F)F)nc32)CC1 ; CC(C)(C)[Si](C)(C)O[C@H]1CC[C@H](n2cc([C@H]3CC[C@H](C=O)CC3)c3cnc(NCCC(F)(F)F)nc32)CC1 ; CC(C)(C)[Si](C)(C)O[C@H]1CC[C@H](n2cc([C@H]3CC[C@@H](C=O)CC3)c3cnc(NCCC(F)(F)F)nc32)CC1]

### Step 1: trans-ethyl-4-[7-[4-hydroxycyclohexyl]-2-[(3,3,3-trifluoropropyl)amino]pyrrolo[2,3-d]pyrimidin-5-yl]cyclohex-3-ene-1-carboxylate

To a stirred solution of trans-4-[5-bromo-2-[(3,3,3-trifluoropropyl)amino]pyrrolo[2,3-d]pyrimidin-7-yl]cyclohexan-1-ol (3.00 g, 7.37 mmol) in 1,4-dioxane (30.0 mL) were added ethyl-4-(4,4,5,5-tetramethyl-1,3,2-dioxaborolan-2-yl)cyclohex-3-ene-1-carboxylate (2.48 g, 8.84 mmol), H₂O (6.00 mL), Pd(PPh₃)₄ (0.85 g, 0.74 mmol) and K₂CO₃ (3.05 g, 22.1 mmol) at room temperature under nitrogen atmosphere. The resulting mixture was purged with nitrogen 3 times and stirred for overnight at 90° C. under nitrogen atmosphere. The resulting mixture was cooled and filtered. The filter cake was washed with 1,4-dioxane (3×10.0 mL). The combined filtrates were concentrated under reduced pressure. The residue was purified by reversed phase flash chromatography with the following conditions: Column: WelFlash™ C18-I, 20-40 um, 330 g; Eluent A: water (plus 10 mmol/L NH₄HCO₃); Eluent B: ACN; Gradient: 45%-65% B in 20 min; Flow rate: 80 mL/min; Detector: UV 220/254 nm; desired fractions were collected at 53% B and concentrated under reduced pressure to afford trans-ethyl-4-[7-[4-hydroxycyclohexyl]-2-[(3,3,3-trifluoropropyl)amino]pyrrolo[2,3-d]pyrimidin-5-yl]cyclohex-3-ene-1-carboxylate (800 mg, 23%) as a white solid. ¹H NMR (400 MHz, DMSO-d₆) δ 8.77 (s, 1H), 7.21 (s, 1H), 6.97 (t, J=5.9 Hz, 1H), 6.15 (s, 1H), 4.66 (d, J=4.5 Hz, 1H), 4.41-4.28 (m, 1H), 4.19-4.04 (m, 2H), 3.63-3.46 (m, 3H), 2.66-2.54 (m, 2H), 2.48-2.29 (m, 4H), 2.06 (d, J=13.0 Hz, 1H), 1.95-1.83 (m, 6H), 1.76-1.63 (m, 1H), 1.33 (q, J=11.5 Hz, 2H), 1.21 (t, J=7.1 Hz, 3H). LC/MS (ESI, m/z): [(M+1)]⁺=481.35

### Step 2: trans-ethyl-4-[7-[(4-hydroxycyclohexyl]-2-[(3,3,3-trifluoropropyl)amino]pyrrolo[2,3-d]pyrimidin-5-yl]cyclohexane-1-carboxylate

To a stirred solution of trans-ethyl -4-[7-[4-hydroxycyclohexyl]-2-[(3,3,3-trifluoropropyl)amino]pyrrolo[2,3-d]pyrimidin-5-yl]cyclohex-3-ene-1-carboxylate (1.00 g, 2.08 mmol) in THF (10.0 mL) was added 10% palladium on activated carbon (200 mg) at room temperature. The resulting mixture was purged with hydrogen 3 times and stirred for 24 h at 50° C. under hydrogen atmosphere. The resulting mixture was filtered. The filter cake was washed with THF (3×10.0 mL). The combined filtrates was concentrated under reduced pressure to afford trans-ethyl-4-[7-[4-hydroxycyclohexyl]-2-[(3,3,3-trifluoropropyl)amino]pyrrolo[2,3-d]pyrimidin-5-yl]cyclohexane-1-carboxylate (1.00 g, 99%) as a colorless oil. ¹H NMR (400 MHz, DMSO-d₆) δ 8.55 (m, 1H), 6.93-6.79 (m, 2H), 4.65 (dd, J=4.5, 2.1 Hz, 1H), 4.40-4.27 (m, 1H), 4.20-3.98 (m, 2H), 3.59-3.43 (m, 3H), 2.80-2.65 (m, 2H), 2.57 (dd, J=11.6, 7.2 Hz, 2H), 2.08-1.74 (m, 13H), 1.70-1.55(m, 2H), 1.55-1.46 (m, 1H), 1.20 (td, J=7.1, 1.4 Hz, 3H). LC/MS (ESI, m/z): [(M+1)]⁺=483.35

### Step 3: trans-ethyl-4-[7-[4-hydroxycyclohexyl]-2-[(3,3,3-trifluoropropyl)amino]pyrrolo[2,3-d]pyrimidin-5-yl]cyclohexane-1-carboxylate

To a stirred mixture of trans-ethyl-4-[7-[4-hydroxycyclohexyl]-2-[(3,3,3-trifluoropropyl)amino]pyrrolo[2,3-d]pyrimidin-5-yl]cyclohexane-1-carboxylate (552 mg, 1.14 mmol) and imidazole (389 mg, 5.72 mmol) in DCM (6.00 mL) was added a solution of TBS-Cl (517 mg, 3.43 mmol) in DCM (4.00 mL). The resulting mixture was stirred for 1 h at room temperature under nitrogen atmosphere. The resulting solution was diluted with water (10.0 mL) and extracted with DCM (3×10.0 mL). The combined organic layer was washed with brine (20.0 mL), dried over anhydrous Na₂SO₄ and filtered. The filtrate was concentrated under reduced pressure. The residue was purified by silica gel column chromatography, eluted with 10% ethyl acetate in petroleum ether to afford trans-ethyl-4-[7-[4-[(tert-butyldimethylsilyl)oxy]cyclohexyl]-2-[(3,3,3-trifluoropropyl)amino]pyrrolo[2,3-d]pyrimidin-5-yl]cyclohexane-1-carboxyl ate (450 mg, 66%) as a white oil. ¹H NMR (400 MHz, DMSO-d₆) δ 8.55 (m, 1H), 6.96-6.75 (m, 2H), 4.36-4.22 (m, 1H), 4.09 (dq, J=10.9, 7.1 Hz, 2H), 3.73 (t, J=9.9 Hz, 1H), 3.52 (q, J=6.7 Hz, 2H), 2.75-2.68 (m, 2H), 2.63-2.54 (m, 2H), 2.08-1.75 (m, 10H), 1.70-1.58 (m, 3H), 1.49-1.38 (m, 3H), 1.20 (t, J=7.1 Hz, 3H), 0.88 (s, 9H), 0.07 (s, 6H). LC/MS (ESI, m/z): [(M+1)]⁺=597.45

### Step 4: trans-(4-[7-[4-[(tert-butyldimethylsilyl)oxy]cyclohexyl]-2-[(3,3,3-trifluoropropyl)amino]pyrrolo[2,3-d]pyrimidin-5-yl]cyclohexyl)methanol

To a stirred solution of ethyl trans-4-[7-[4-[(tert-butyldimethylsilyl)oxy]cyclohexyl]-2-[(3,3,3-trifluoropropyl)amino]pyrrolo[2,3-d]pyrimidin-5-yl]cyclohexane-1-carboxylate (1.00 g, 1.68 mmol) in THF (10.0 mL) was added LiAlH₄ (1 Min THF, 2.50 mL, 2.51 mmol) dropwise at 0° C. under nitrogen atmosphere. The reaction was stirred for 2 h at room temperature. The reaction was quenched with water (1.00 mL) at 0° C. Then NaOH aq. (15%) was added. Finally, water (3.00 mL) was added. The resulting mixture was filtered. The filter cake was washed with THF (3×3.00 mL). The combined filtrates was concentrated under reduced pressure to afford trans-(4-[7-[4-[(tert-butyldimethylsilyl)oxy]cyclohexyl]-2-[(3,3,3-trifluoropropyl)amino]pyrrolo[2,3-d]pyrimidin-5-yl]cyclohexyl)methanol (900 mg, 97%) as a white semi-solid. ¹H NMR (400 MHz, DMSO-d₆) δ 8.54 (d, J=12.8 Hz, 1H), 6.96-6.80 (m, 2H), 4.47-4.24 (m, 2H), 3.79-3.68 (m, 1H), 3.65-3.59 (m, 1H), 3.52 (q, J=7.0 Hz, 2H), 3.39 (t, J=6.2 Hz, 1H), 2.63-2.54 (m, 2H), 2.05-1.89 (m, 4H), 1.86-1.62 (m, 8H), 1.56 (d, J=7.9 Hz, 2H), 1.48-1.31 (m, 4H), 0.88 (s, 9H), 0.07 (s, 6H). LC/MS (ESI, m/z): [(M+1)]⁺=555.30.

### Step 5: trans-4-[7-[trans-4-[(tert-butyldimethylsilyl)oxy]cyclohexyl]-2-[(3,3,3-trifluoropropyl)amino]pyrrolo[2,3-d]pyrimidin-5-y]cyclohexane-1-carbaldehyde and cis-4-[7-[trans-4-[(tert-butyldimethylsilyl)oxy]cyclohexyl]-2-[(3,3,3-trifluoropropyl)amino]pyrrolo[2,3-d]pyrimidin-5-yl]cyclohexane-1-carbaldehyde

To a stirred solution of trans-4-[5-[4-(hydroxymethyl)cyclohexyl]-2-[(3,3,3-trifluoropropyl)amino]pyrrolo[2,3-d]pyrimidin-7-yl]cyclohexan-1-ol (600 mg, 1.36 mmol) in DCM (7.00 mL) was added Dess-Martin reagent (520 mg, 1.23 mmol) at 0° C. The resulting mixture was stirred for 1 h at 0° C. under nitrogen atmosphere. The resulting mixture was concentrated under vacuum. The residue was purified by reversed phase flash chromatography with the following conditions: Column: WelFlash™ C18-I, 20-40 um, 330 g; Eluent A: water (plus 10 mmol/L HOAc); Eluent B: ACN; Gradient: 50%-70% B in 15 min; Flow rate: 80 mL/min; Detector: UV 220/254 nm; desired fractions were collected at 95% B and concentrated under reduced pressure to afford trans-4-[7-[trans-4-[(tert-butyldimethylsilyl)oxy]cyclohexyl]-2-[(3,3,3-trifluoropropyl)amino]pyrrolo[2,3-d]pyrimidin-5-yl]cyclohexane-1-carbaldehyde (180 mg, 24%) as a light yellow solid and cis-4-[7-[trans-4-[(tert-butyldimethylsilyl)oxy]cyclohexyl]-2-[(3,3,3-trifluoropropyl)amino]pyrrolo[2,3-d]pyrimidin-5-yl]cyclohexane-1-carbaldehyde (60 mg, 8%) as a light yellow solid.

Intermediate L6: ¹H NMR (400 MHz, CDCl₃) δ 9.78 (s, 1H), 8.47 (s, 1H), 6.60 (s, 1H), 5.32 (s, 1H), 4.38 (d, J=12.6 Hz, 1H), 3.83-3.59 (m, 2H), 2.84-2.70 (m, 1H), 2.61-2.44 (m, 3H), 2.29 (d, J=13.7 Hz, 2H), 2.11-1.93 (m, 5H), 1.87-1.77 (m, 3H), 1.60-1.50 (m, 4H), 1.28 (s, 2H), 0.93 (s, 9H), 0.11 (s, 6H). LC/MS (ESI, m/z): [(M+1)]⁺=553.35

Intermediate L7: ¹H NMR (400 MHz, CDCl₃) δ 9.71 (d, J=1.6 Hz, 1H), 8.51 (s, 1H), 6.66 (s, 1H), 5.44 (s, 1H), 4.48-4.35 (m, 1H), 3.79-3.62 (m, 3H), 2.69 (s, 1H), 2.55-2.50 (m, 2H), 2.18 (d, J=9.8 Hz, 2H), 2.03 (d, J=12.1 Hz, 4H), 1.86 (d, J=12.4 Hz, 2H), 1.56 (d, J=11.3 Hz, 4H), 1.31-1.23 (m, 4H), 0.93 (s, 9H), 0.11 (s, 6H). LC/MS (ESI, m/z): [(M+1)]⁺=553.25

### INTERMEDIATE M1: tert-butyl 4-[1-[4-(4,4,5,5-tetramethyl-1,3,2-dioxaborolan-2-yl)phenyl]ethyl]piperazine-1-carboxylate

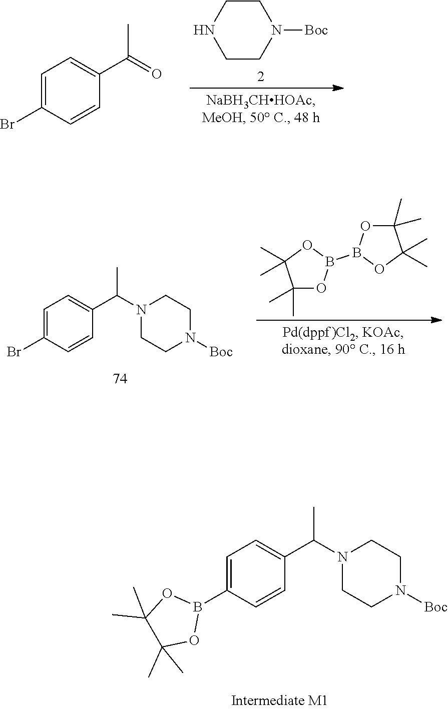

### Step 1: tert-butyl 4-[1-(4-bromophenyl)ethyl]piperazine-1-carboxylate

A solution of 1-(4-bromophenyl)ethan-1-one (5.00 g, 25.1 mmol), tert-butyl piperazine-1-carboxylate (4.68 g, 25.1 mmol) and AcOH (4.32 mL) in MeOH (50.0 mL) was stirred for 30 min at room temperature under nitrogen atmosphere followed by the addition of NaBH₃CN (3.16 g, 50.2 mmol) at 0° C. The resulting mixture was stirred for 48 h at 50° C. under nitrogen atmosphere. The resulting solution was cooled down to room temperature and filtered. The filtrate was concentrated under reduced pressure. The residue was purified by reversed phase flash chromatography with the following conditions: WelFlash™ C18-I, 20-40 um, 330 g; Eluent A: water (plus 10 mmol/L NH₄HCO₃); Eluent B: ACN; Gradient: 60%-99% B in 15 min; Flow rate: 80 mL/min; Detector: UV 220/254 nm; desired fractions were collected at 99% B and concentrated under reduced pressure to afford tert-butyl 4-[1-(4-bromophenyl)ethyl]piperazine-1-carboxylate (4.60 g, 50%) as a brown oil. ¹H NMR (400 MHz, CDCl₃) δ 7.53-7.38 (m, 2H), 7.25-7.12 (m, 2H), 3.45-3.25 (m, 5H), 2.49-2.35 (m, 2H), 2.35-2.12 (m, 2H), 1.46 (s, 9H), 1.34 (d, J=6.6 Hz, 3H). LC/MS (ESI, m/z): [(M+1)]⁺=369.05, 371.05

The following intermediates in Table 4 were synthesized according to the above procedure:

TABLE 4

| Inter- | | | MS: | |
|---|---|---|---|---|
| mediate | Structure | Name | [(M + 1)]⁺ | ¹H NMR |
| 75 | 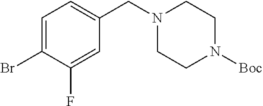 [SMILES: CC(C)(C)OC(=O)N1CCN(Cc2ccc(Br)c(F)c2)CC1] | tert-butyl 4-[(4- bromo-3-fluoro- phenyl)methyl]- piperazine-1- carboxylate | 373.05, 375.05 | (400 MHz, CDCl₃) δ 7.54-7.41 (m, 1H), 7.15 (dd, J = 9.5, 1.9 Hz, 1H), 7.08-6.92 (m, 1H), 3.46 (s, 2H), 3.43 (t, J = 5.1 Hz, 4H), 2.46-2.30 (m, 4H), 1.46 (s, 9H) |

### Step 2: tert-butyl 4-[1-[4-(4,4,5,5-tetramethyl-1,3,2-dioxaborolan-2-yl)phenyl]ethyl]piperazine-1-carboxylate

To a stirred solution of tert-butyl 4-[1-(4-bromophenyl)ethyl]piperazine-1-carboxylate (4.60 g, 12.5 mmol) and 4,4,5,5-tetramethyl-2-(4,4,5,5-tetramethyl-1,3,2-dioxaborolan-2-yl)-1,3,2-dioxaborolane (6.33 g, 24.9 mmol) in 1,4-dioxane (40.0 mL) was added Pd(dppf)Cl₂.CH₂Cl₂ (509 mg, 0.62 mmol) and KOAc (2.44 g, 24.9 mmol) at room temperature under nitrogen atmosphere. The resulting mixture was purged with nitrogen for 3 times and was stirred for 16 h at 90° C. The mixture was cooled down to room temperature and diluted with H₂O (100 mL). The resulting mixture was extracted with EtOAc (3×150 mL). The combined organic layers was washed with brine (200 mL) and dried over anhydrous Na₂SO₄. After filtration, the filtrate was concentrated under reduced pressure. The residue was purified by silica gel column chromatography, eluted with 3%˜10% ethyl acetate in petroleum ether to afford tert-butyl 4-[1-[4-(4,4,5,5-tetramethyl-1,3 ,2-dioxaborolan-2-yl)phenyl] ethyl] piperazine-1-carboxylate (4.80 g, 93%) as a light brown solid. ¹H NMR (400 MHz, CDCl₃) δ 7.77 (d, J=7.5 Hz, 2H), 7.32 (d, J=7.5 Hz, 2H), 3.44-3.31 (m, 5H), 2.49-2.35 (m, 2H), 2.38-2.25 (m, 2H), 1.43 (s, 9H), 1.34 (s, 12H), 1.27 (s, 9H), 1.24 (s, 3H). LC/MS (ESI, m/z): [(M+1)]⁺=417.15

The following intermediates in Table 5 were synthesized according to the above procedure:

TABLE 5

| Inter- | | | MS: | |
|---|---|---|---|---|
| mediate | Structure | Name | [(M + 1)]⁺ | ¹H NMR |
| M2 | 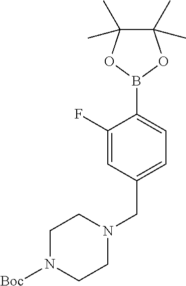 [SMILES: CC(C)(C)OC(=O)N1CCN(Cc2ccc(B3OC(C)(C)C(C)(C)O3)c(F)c2)CC1] | tert-butyl 4-[[3-fluoro- 4-(4,4,5,5-tetramethyl- 1,3,2-dioxaborolan-2- yl)phenyl]methyl]pipe razine-1-carboxylate | 421.35 | ¹H NMR (400 MHz, CDCl₃) δ 7.70 (t, J = 7.0 Hz, 1H), 7.19- 7.01 (m, 2H), 3.54 (s, 2H), 3.55-3.46 (m, 4H), 2.50-2.33 (m, 4H), 1.47 (s, 9H), 1.38 (s, 12H) |

### INTERMEDIATE M3: tert-butyl 4-[[4-(4,4,5,5-tetramethyl-1,3,2-dioxaborolan-2-yl)cyclohex-3-en-1-yl]methyl]piperazine-1-carboxylate

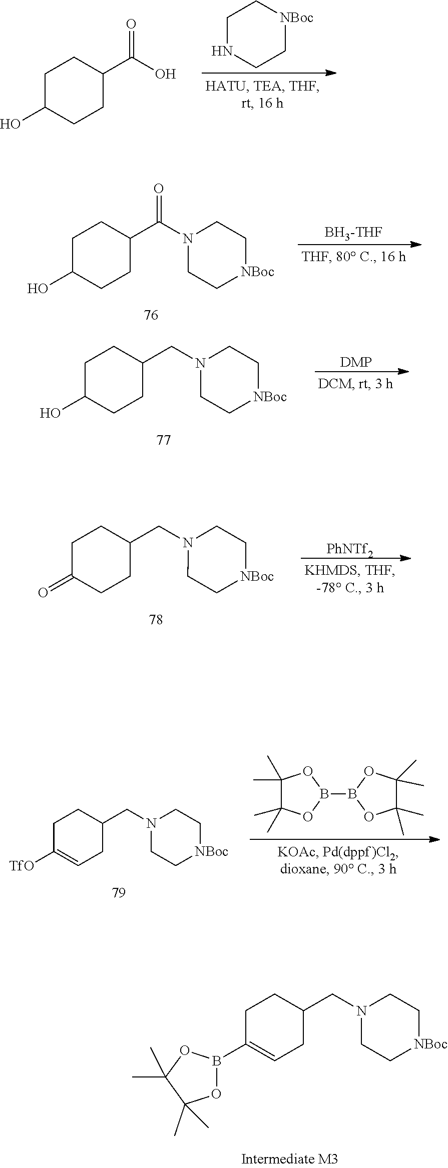

### Step 1: tert-butyl 4-(4-hydroxycyclohexanecarbonyl)piperazine-1-carboxylate

To a stirred solution of 4-hydroxycyclohexane-1-carboxylic acid (50.0 g, 347 mmol) in THF (1.00 L) was added HATU (171 g, 451 mmol) in portions at 0° C. The resulting solution was stirred for 30 min at 0° C. under nitrogen atmosphere. To the above solution were added tert-butyl piperazine-1-carboxylate (71.1 g, 382 mmol) and TEA (70.2 g, 694 mmol) at 0° C. under nitrogen atmosphere. The resulting solution was stirred for 16 h at room temperature. The resulting mixture was concentrated under reduced pressure and the residue was diluted with water (1.50 L), extracted with DCM (3×1.40 L). The combined organic layers were washed with brine (1.40 L) and dried over anhydrous Na₂SO₄. After filtration, the filtrate was concentrated under reduced pressure. The residue was triturated with EtOAc (500 mL). The precipitated solids were collected by filtration and washed with EtOAc (3×100 mL) to afford tert-butyl 4-(4-hydroxycyclohexanecarbonyl)piperazine-1-carboxylate (85.0 g, 78%) as a white solid. ¹H NMR (400 MHz, CDCl₃) δ 3.72-3.38 (m, 9H), 2.58-2.37 (m, 1H), 2.13-1.55 (m, 6H), 1.49 (s, 9H), 1.38-1.24 (m, 2H). LC/MS (ESI, m/z): [(M+1)]⁺=313.20

### Step 2: tert-butyl 4-[(4-hydroxycyclohexyl)methyl]piperazine-1-carboxylate

To a solution of tert-butyl 4-(4-hydroxycyclohexanecarbonyl)piperazine-1-carboxylate (85.0 g, 272 mmol) in THF (1.00 L) was added BH₃-THF (1.00 L, 1 Min THF) dropwise at 0° C. The solution was stirred at 80° C. for 16 h. The resulting mixture was cooled to 0° C. followed by the addition of MeOH (500 mL) at 0° C. slowly. The mixture was stirred at room temperature for 30 min and concentrated under reduced pressure. The residue was diluted with H₂O (1.00 L) and extracted with CH₂Cl₂ (3×1.00 L). The combined organic layers were washed with brine (3×1.00 L) and dried over anhydrous Na₂SO₄. After filtration, the filtrate was concentrated under reduced pressure to afford tert-butyl 4-[(4-hydroxycyclohexyl)methyl]piperazine-1-carboxylate (80.0 g, 98%) as a white solid. ¹H NMR (400 MHz, CDCl₃) δ 4.04-3.34 (m, 5H), 3.01 (d, J=11.9 Hz, 2H), 2.74-2.51 (m, 3H), 2.13-1.94 (m, 3H), 1.75-1.57 (m, 3H), 1.48 (d, J=2.6 Hz, 9H), 1.40-1.06 (m, 4H). LC/MS (ESI, m/z): [(M+1)]⁺=299.25

### Step 3: tert-butyl 4-((4-oxocyclohexyl)methyl)piperazine-1-carboxylate

To a solution of tert-butyl 4-[(4-hydroxycyclohexyl)methyl]piperazine-1-carboxylate (104 g, 348 mmol) in DCM (2.00 L) was added Dess-Martin reagent (222 g, 523 mmol) at 0° C. The mixture was stirred at room temperature for 3 h. The resulting mixture was quenched with saturated NaHCO₃ (aq.) and Na₂S₂O₃ (aq. v/v=1/1) at 0° C. The resulting mixture was extracted with CH₂Cl₂ (3×1.00 L). The combined organic layers were washed with brine (3×1.00 L) and dried over anhydrous Na₂SO₄. After filtration, the filtrate was concentrated under reduced pressure to afford tert-butyl 4-[(4-oxocyclohexyl)methyl]piperazine-1-carboxylate (80.0 g, 77%) as a light yellow solid. ¹H NMR (400 MHz, DMSO-d₆) δ 3.30 (d, J=5.0 Hz, 4H), 2.43-2.26 (m, 6H), 2.25-2.11 (m, 4H), 2.06-1.91 (m, 3H), 1.39 (s, 9H), 1.28 (q, J=12.5, 11.1 Hz, 2H). LC/MS (ESI, m/z): [(M+1)]⁺=297.35

### Step 4: tert-butyl 4-[[4-(trifluoromethanesulfonyloxy)cyclohex-3-en-1-yl]methyl]piperazine-1-carboxylate

To a solution of tert-butyl 4-[(4-oxocyclohexyl)methyl]piperazine-1-carboxylate (88.0 g, 297 mmol) and 1,1,1-trifluoro-N-phenyl-N-(trifluoromethane)sulfonylmethanesulfonamide (149 g, 416 mmol) in THF (1.00 L) was added KHMDS (416 mL, 1 Min THF) dropwise at −78° C. The mixture was stirred at −78° C. for 3 h. The resulting mixture was quenched with H₂O (500 mL) and extracted with CH₂Cl₂ (3×1.00 L). The combined organic layers were washed with brine (3×1.00 L) and dried over anhydrous Na₂SO₄. After filtration, the filtrate was concentrated under reduced pressure. The residue was purified by silica gel column chromatography, eluted with 10%˜50% ethyl acetate in petroleum ether to afford tert-butyl 4-[[4-(trifluoromethanesulfonyloxy)cyclohex-3-en-1-yl]methyl]piperazine-1-carboxylate (89.0 g, 70%) as a light yellow oil. ¹H NMR (400 MHz, DMSO-d₆) δ 5.87 (dt, J=5.0, 2.3 Hz, 1H), 3.30 (t, J=5.1 Hz, 4H), 2.39-2.31 (m, 1H), 2.29 (t, J=5.1 Hz, 6H), 2.18 (d, J=6.7 Hz, 2H), 1.92-1.83 (m, 3H), 1.47-1.40 (m, 1H), 1.40 (s, 9H) LC/MS (ESI, m/z): [(M+1)]⁺=429.10

### Step 5: tert-butyl 4-[[4-(4,4,5,5-tetramethyl-1,3,2-dioxaborolan-2-yl)cyclohex-3-en-1-yl]methyl]piperazine-1-carboxylate

To a solution of tert-butyl 4-[[4-(trifluoromethanesulfonyloxy)cyclohex-3-en-1-yl]methyl]piperazine-1-carboxylate (89.0 g, 208 mmol) and 4,4,5,5-tetramethyl-2-(4,4,5,5-tetramethyl-1,3,2-dioxaborolan-2-yl)-1,3,2-dioxaborolane (106 g, 416 mmol) in 1,4-dioxane (1.20 L) were added KOAc (40.8 g, 416 mmol) and Pd(dppf)C12-CH₂Cl₂ (17.0 g, 20.8 mmol). After stirring for 3 h at 90° C. under a nitrogen atmosphere, the resulting mixture was cooled and concentrated under reduced pressure. The residue was purified by silica gel column chromatography, eluted with 10%˜50% ethyl acetate in petroleum ether to afford tert-butyl 4-[[4-(4,4,5,5-tetramethyl-1,3,2-dioxaborolan-2-yl)cyclohex-3-en-1-yl]methyl]piperazine-1-carboxylate (60.0 g, 71%) as a light yellow oil. ¹H NMR (400 MHz, CD₃OD) δ 6.52 (dq, J=4.3, 2.3 Hz, 1H), 3.44 (t, J=5.1 Hz, 4H), 2.45-2.32 (m, 4H), 2.29-2.15 (m, 3H), 2.14-1.97 (m, 2H), 1.90-1.75 (m, 4H), 1.47 (s, 9H), 1.26 (d, J=1.3 Hz, 12H). LC/MS (ESI, m/z): [(M+1)]⁺=407.40

### INTERMEDIATE M4: trans-N-butyl-7-[4-[(tert-butyldimethylsilyl)oxy]cyclohexyl]-5-(4,4,5,5-tetramethyl-1,3,2-dioxaborolan-2-yl)-7H-pyrrolo[2,3-d]pyrimidin-2-amine

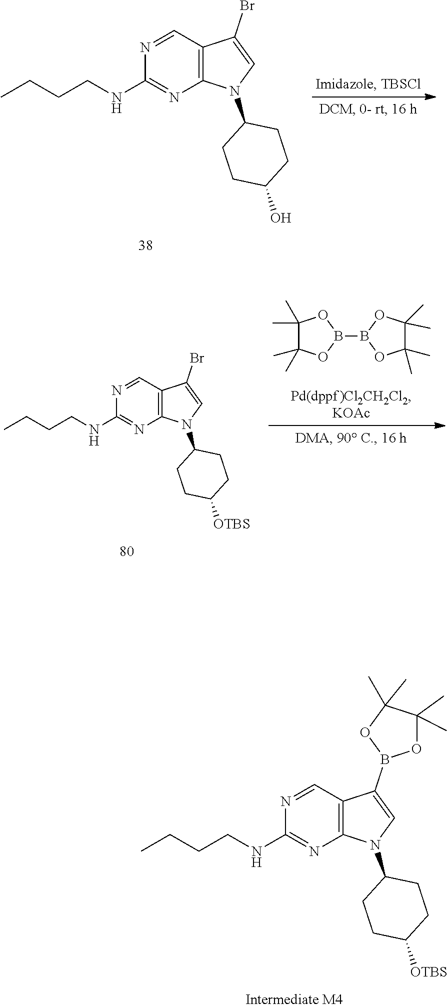

Step 1: 5-bromo-N-butyl-7-[trans-4-[(tert-butyldimethylsilyl)oxy]cyclohexyl]-7H-pyrrolo[2,3-d]pyrimidin-2-amine

To a stirred solution of trans-4-[5-bromo-2-(butylamino)-7H-pyrrolo[2,3-d]pyrimidin-7-yl]cyclohexan-1-ol (5.40 g, 14.7 mmol) and imidazole (4.00 g, 58.8 mmol) in DCM (100 mL) was added TBSCl (24.4 g, 162 mmol) in portions at 0° C. under nitrogen atmosphere. The resulting mixture was stirred for 16 h at room temperature. The resulting mixture was diluted with sat. NaHCO₃ (500 mL) and extracted with DCM (3×300 mL). The combined organic layers were washed with brine (400 mL) and dried over anhydrous Na₂SO₄. After filtration, the filtrate was concentrated under reduced pressure. The residue was purified by silica gel column chromatography, eluted with 1%˜2% methanol in dichloromethane to afford trans-5-bromo-N-butyl-7-[4-[(tert-butyldimethylsilyl)oxy]cyclohexyl]-7H-pyrrolo[2,3-d]pyrimidin-2-amine (5.00 g, 71%) as a light pink solid. ¹H NMR (400 MHz, CD₃OD) δ 8.42 (s, 1H), 7.50 (d, J=3.6 Hz, 1H), 4.61-4.40 (m, 1H), 3.89-3.73 (m, 1H), 3.50 (t, J=7.2 Hz, 2H), 2.11-1.94 (m, 6H), 1.75-1.63 (m, 2H), 1.63-1.42 (m, 4H), 1.03 (td, J=7.4, 1.8 Hz, 3H), 0.94 (d, J=1.8 Hz, 9H), 0.13 (d, J=1.7 Hz, 6H). LC/MS (ESI, m/z): [(M+1)]⁺=481.30, 483.30

### Step 2: trans-N-butyl-7-[(4-[(tert-butyldimethylsilyl)oxy]cyclohexyl]-5-(4,4,5,5-tetramethyl-1,3,2-dioxaborolan-2-yl)-7H-pyrrolo[2,3-d]pyrimidin-2-amine

To a stirred solution of trans-5-bromo-N-butyl-7-[4-[(tert-butyldimethylsilyl)oxy]cyclohexyl]-7H-pyrrolo[2,3-d]pyrimidin-2-amine (3.00 g, 6.23 mmol) and 4,4,5,5-tetramethyl-2-(4,4,5,5-tetramethyl-1,3,2-dioxaborolan-2-yl)-1,3,2-dioxaborolane (7.91 g, 31.1 mmol) in DMA (20.0 mL) were added Pd(dppf)Cl₂.CH₂Cl₂ (509 mg, 0.62 mmol) and KOAc (1.22 g, 12.5 mmol) at room temperature under nitrogen atmosphere. The resulting mixture was purged with nitrogen for 3 times and was stirred for 16 h at 90° C. The mixture was cooled down to room temperature and concentrated under reduced pressure. The residue was purified by silica gel column chromatography, eluted with 1%˜15% ethyl acetate in petroleum ether to afford trans-N-butyl-7-[4-[(tert-butyldimethylsilyl)oxy]cyclohexyl]-5-(4,4,5,5-tetramethyl-1,3,2-dioxaborolan-2-yl)-7H-pyrrolo[2,3-d]pyrimidin-2-amine (1.00 g, 23%) as a brown solid. ¹H NMR (400 MHz, CD₃OD) δ 8.39 (s, 1H), 7.11-7.05 (m, 1H), 4.31 (t, J=6.6 Hz, 1H), 3.89-3.73 (m, 1H), 3.43 (t, J=7.1 Hz, 2H), 2.09-1.95 (m, 6H), 1.78-1.60 (m, 2H), 1.56-1.45 (m, 4H), 1.22 (s, 12H), 1.09-0.98 (m, 3H), 0.94 (s, 9H), 0.13 (s, 6H). LC/MS (ESI, m/z): [(M+1)]⁺=529.35

### INTERMEDIATE N: tert-butyl 4-[(6-chloropyridazin-3-yl)methyl]piperazine-1-carboxylate

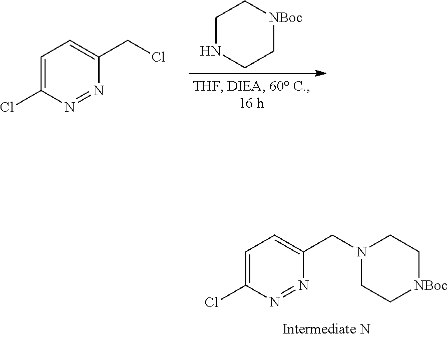 [SMILES: ClCc1ccc(Cl)nn1 ; CC(C)(C)OC(=O)N1CCNCC1 ; CC(C)(C)OC(=O)N1CCN(Cc2ccc(Cl)nn2)CC1]

### tert-butyl 4-[(6-chloropyridazin-3-yl)methyl]piperazine-1-carboxylate

To a stirred solution of 3-chloro-6-(chloromethyl)pyridazine (3.00 g, 18.4 mmol) and tert-butyl piperazine-1-carboxylate (4.11 g, 22.1 mmol) in THF (30.0 mL) was added DIEA (7.14 g, 55.2 mmol) at room temperature under nitrogen atmosphere. The solution was allowed to react at 60° C. for 16 h. Upon completion, the mixture was cooled down to room temperature and concentrated under reduced pressure. The residue was purified by silica gel column chromatography, eluted with 60% ethyl acetate in petroleum ether to afford tert-butyl 4-[(6-chloropyridazin-3-yl)methyl]piperazine-1-carboxylate (2.5. g, 43%) as an off-white solid. ¹H NMR (400 MHz, DMSO-d₆) δ 7.91 (d, J=8.8 Hz, 1H), 7.80 (d, J=8.8 Hz, 1H), 3.81 (s, 2H), 3.33-3.21 (m, 4H), 2.48-2.31 (m, 4H), 1.40 (s, 9H). LC/MS (ESI, m/z): [(M+1)]⁺=313.20

### INTERMEDIATE O1: trans-4-[5-[trans-4-(piperazin-1-ylmethyl)cyclohexyl]-2-[(3,3,3-trifluoropropyl)amino]pyrrolo[2,3-d]pyrimidin-7-yl]cyclohexan-1-ol hydrochloride

### INTERMEDIATE O2: trans-4-[5-[cis-4-(piperazin-1-ylmethyl)cyclohexyl]-2-[(3,3,3-trifluoropropyl)amino]pyrrolo[2,3-d]pyrimidin-7-yl]cyclohexan-1-ol hydrochloride

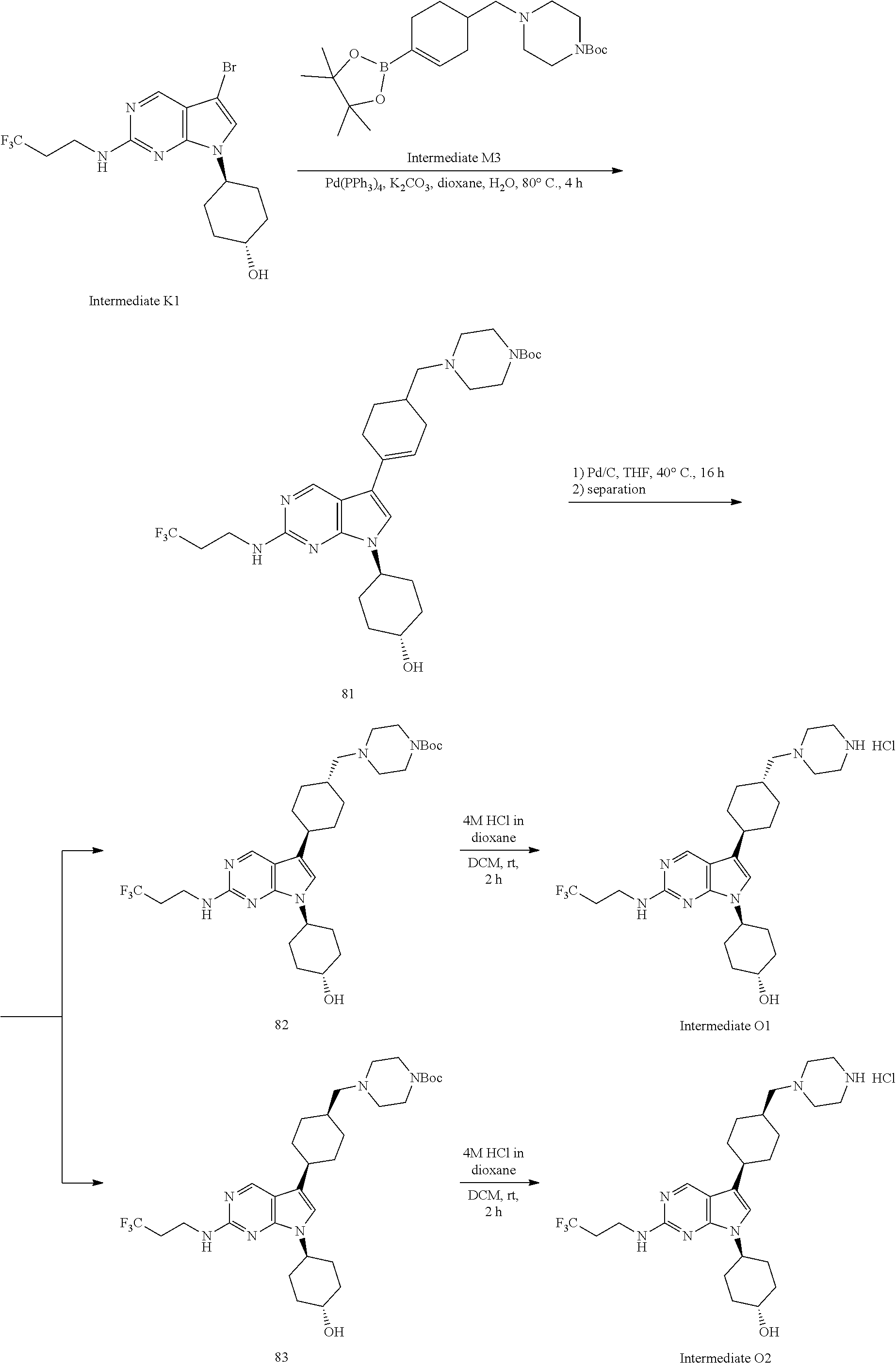

### Step 1: tert-butyl 4-[(4-[7-[trans-4-hydroxycyclohexyl]-2-[(3,3,3-trifluoropropyl)amino]pyrrolo[2,3-d]pyrimidin-5-yl]cyclohex-3-en-1-yl)methyl]piperazine-1-carboxylate

To a stirred solution of trans-4-[5-bromo-2-[(3,3,3-trifluoropropyl)amino]pyrrolo[2,3-d]pyrimidin-7-yl]cyclohexan-1-ol (640 mg, 1.57 mmol,) and tert-butyl 4-[[4-(4,4,5,5-tetramethyl-1,3,2-dioxaborolan-2-yl)cyclohex-3-en-1-yl]methyl]piperazine-1-carboxylate (830 mg, 2.04 mmol) in 1,4-dioxane (15.0 mL) and H₂O (5.00 mL) were added K₂CO₃ (652 mg, 4.72 mmol) and Pd(PPh₃)₄ (182 mg, 0.16 mmol) at room temperature. The resulting solution was purged with nitrogen three times and stirred for 4 h at 80° C. under nitrogen atmosphere. The resulting mixture was cooled down to room temperature and diluted with water (20.0 mL). The resulting mixture was extracted with EtOAc (3×50.0 mL). The combined organic layers were washed with brine (50.0 mL) and dried over anhydrous Na₂SO₄. After filtration, the filtrate was concentrated under reduced pressure. The residue was purified by reversed phase flash chromatography with the following conditions: Column: WelFlash™ C18-I, 20-40 um, 330 g; Eluent A: water (plus 10 mmol/L NH₄HCO₃); Eluent B: ACN; Gradient: 70%-99% B in 20 min; Flow rate: 80 mL/min; Detector: UV 220/254 nm; desired fractions were collected at 99% B and concentrated under reduced pressure to afford tert-butyl 4-[(4-[7-[trans-4-hydroxycyclohexyl]-2-[(3,3,3-trifluoropropyl)amino]pyrrolo[2,3-d]pyrimidin-5-yl]cyclohex-3-en-1-yl)methyl]piperazine-1-carboxylate (300 mg, 31%) as a yellow solid. ¹H NMR (400 MHz, CD₃OD) δ 8.66 (s, 1H), 7.08 (s, 1H), 6.15 (s, 1H), 4.54-4.42 (m, 1H), 3.75-3.64 (m, 3H), 3.49-3.41 (m, 4H), 2.64-2.49 (m, 2H), 2.49-2.36 (m, 6H), 2.33 (d, J=6.0 Hz, 2H), 2.12 (d, J=12.9 Hz, 2H), 2.06-1.86 (m, 8H), 1.59-1.46 (m, 3H), 1.48 (s, 9H). LC/MS (ESI, m/z): [(M+1)]⁺=607.35

The following intermediates in Table 6 were synthesized according to the above procedure:

TABLE 6

| Inter- | | | MS: | |
|---|---|---|---|---|
| mediate | Structure | Name | [(M + 1)]⁺ | ¹H NMR |
| 81-1 | 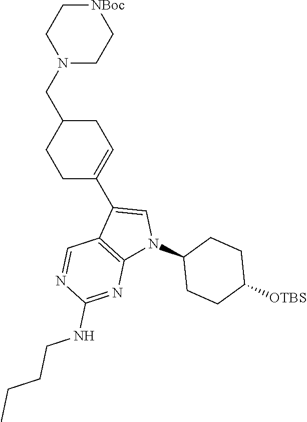 [SMILES: CCCCNc1ncc2c(C3=CCC(CN4CCN(C(=O)OC(C)(C)C)CC4)CC3)cn([C@H]3CC[C@H](O[Si](C)(C)C(C)(C)C)CC3)c2n1] | tert-butyl-4-([4-[2- (butylamino)-7-[4- [(trans-tert-butyl- dimethylsilyl)oxy]- cyclohexyl]-7H- pyrrolo[2,3-d]- pyrimidin-5-yl]- cyclohex-3-en-1- yl]methyl)pipera- zine-1-carboxylate | 681.50 | ¹H NMR (400 MHz, DMSO- d₆) δ 8.70 (s, 1H), 7.74-7.67 (m, 1H), 7.15 (s, 1H), 6.12 (s, 1H), 4.35 (t, J = 12.0 Hz, 1H), 4.23 (t, J = 6.5 Hz, 1H), 2.35- 2.15 (m, 10H), 2.04-1.80 (m, 10H), 1.66-1.61 (m, 1H), 1.58-1.43 (m, 4H), 1.52-1.31 (m, 9H), 1.29-1.18 (m, 6H), 0.93 (d, J = 7.3 Hz, 3H), 0.88 (d, J = 4.0 Hz, 9H), 0.08 (s, 6H) |
| 81-2 | 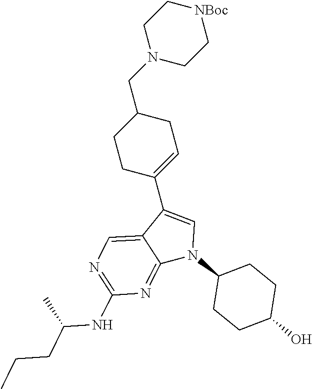 [SMILES: CCC[C@H](C)Nc1ncc2c(C3=CCC(CN4CCN(C(=O)OC(C)(C)C)CC4)CC3)cn([C@H]3CC[C@H](O)CC3)c2n1] | tert-butyl-4-[(4-[2- [(2S)-pentan-2- ylamino]-7-[trans- 4-hydroxycyclo- hexyl]pyrrolo[2,3- d]pyrimidin-5-yl]- cyclohex-3-en-1- yl)methyl]pipera- zine-1-carboxylate | 581.65 | (400 MHz, CD₃OD) δ 8.61 (s, 1H), 7.03 (s, 1H), 6.13 (s, 1H), 4.49-4.38 (m, 1H), 4.10 (q, J = 6.4 Hz, 1H), 3.77-3.65 (m, 1H), 3.55-3.41 (m, 4H), 3.37 (s, 2H), 2.49-2.36 (m, 5H), 2.32 (d, J = 6.2 Hz, 2H), 2.11 (d, J = 12.3 Hz, 2H), 2.03-1.90 (m, 7H), 1.64 (t, J = 7.4 Hz, 1H), 1.53 (t, J = 7.4 Hz, 3H), 1.48 (s, 9H), 1.49-1.36 (m, 3H), 1.33-1.20 (m, 3H), 0.99 (t, J = 7.2 Hz, 3H) |
| 81-3 | 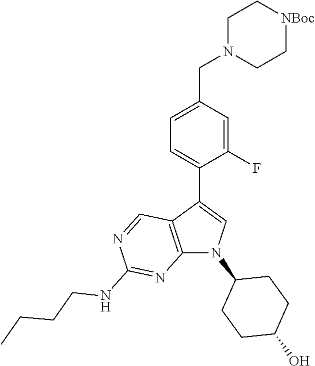 [SMILES: CCCCNc1ncc2c(-c3ccc(CN4CCN(C(=O)OC(C)(C)C)CC4)cc3F)cn([C@H]3CC[C@H](O)CC3)c2n1] | tert-butyl-4-([4-[2- (butylamino)-7- [trans-4-hydroxy- cyclohexyl]-7H- pyrrolo[2,3-d]- pyrimidin-5-yl]- 3-fluorophenyl]- methyl)pipera- zine-1-carboxylate | 581.40 | (400 MHz, CD₃OD) δ 8.58 (s, 1H), 7.69-7.52 (m, 3H), 7.39 (s, 1H), 7.21 (d, J = 9.6 Hz, 1H), 4.55 (s, 1H), 4.35- 4.08 (m, 1H), 3.71 (s, 1H), 3.57 (s, 2H), 3.45 (d, J = 6.7 Hz, 4H), 3.34 (s, 1H), 2.55- 2.42 (m, 3H), 2.20-1.92 (m, 5H), 1.66 (t, J = 7.4 Hz, 2H), 1.60-1.49 (m, 2H), 1.45 (s, 9H), 1.28 (s, 3H), 1.09-0.95 (m, 3H), 0.90 (s, 1H). |
| 81-4 | 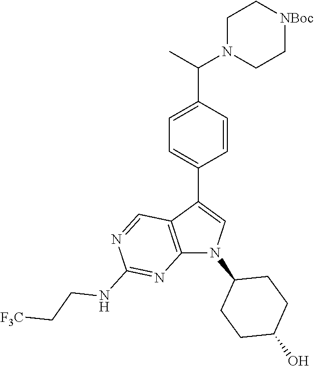 [SMILES: CC(c1ccc(-c2cn([C@H]3CC[C@H](O)CC3)c3nc(NCCC(F)(F)F)ncc23)cc1)N1CCN(C(=O)OC(C)(C)C)CC1] | tert-butyl-4-[1-(4- [7-[trans-4-hydroxy- cyclohexyl]-2- [(3,3,3-trifluoro- propyl)amino]- 7H-pyrrolo[2,3-d]- pyrimidin-5-yl]- phenyl)ethyl]pipera- zine-1-carboxylate | 617.30 | (400 MHz, CD₃OD) δ 8.76 (s, 1H), 7.61 (d, J = 7.8 Hz, 2H), 7.45-7.36 (m, 3H), 4.60-4.52 (m, 1H), 3.72 (t, J = 7.2 Hz, 3H), 3.51-3.36 (m, 6H), 2.61 (s, 2H), 2.50 (d, J = 11.6 Hz, 2H), 2.42 (s, 2H), 2.15 (d, J = 12.1 Hz, 2H), 2.15-2.06 (m, 3H), 1.59-1.46 (m, 2H), 1.55-1.36 (m, 12H). |
| 81-5 | 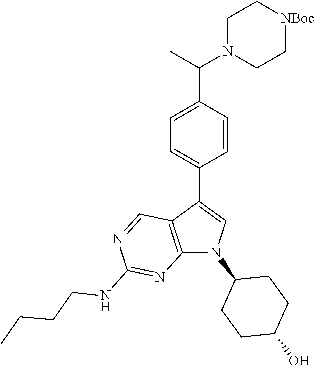 [SMILES: CCCCNc1ncc2c(-c3ccc(C(C)N4CCN(C(=O)OC(C)(C)C)CC4)cc3)cn([C@H]3CC[C@H](O)CC3)c2n1] | tert-butyl-4-(1-[4- [2-(butylamino)-7- [trans-4-hydroxy- cyclohexyl]-7H- pyrrolo[2,3-d]- pyrimidin-5-yl]- phenyl]ethyl)pipera- zine-1-carboxylate | 577.40 | (400 MHz, CD₃OD) δ 8.69 (s, 1H), 7.74-7.53 (m, 4H), 7.40-7.33 (m, 2H), 4.53 (s, 1H), 4.35-4.19 (m, 1H), 4.15-4.06 (m, 1H), 3.71 (s, 1H), 3.49-3.36 (m, 5H), 2.48 (d, J = 10.7 Hz, 2H), 2.38 (d, J = 11.7 Hz, 2H), 2.12 (d, J = 12.6 Hz, 2H), 2.02 (d, J = 12.5 Hz, 4H), 1.72-1.62 (m, 2H), 1.59- 1.45 (m, 3H), 1.43 (s, 9H), 1.41 (s, 1H), 1.30-1.19 (m, 2H), 1.05-0.97 (m, 3H), 0.97-0.89 (m, 1H). |
| 81-6 | 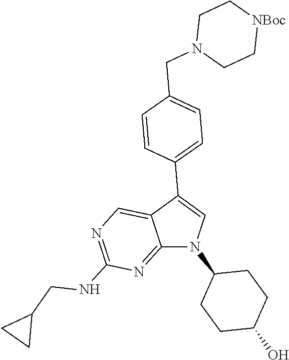 [SMILES: CC(C)(C)OC(=O)N1CCN(Cc2ccc(-c3cn([C@H]4CC[C@H](O)CC4)c4nc(NCC5CC5)ncc34)cc2)CC1] | tert-butyl-4-[(4-[2- [(cyclopropylmeth- yl)amino]-7-[trans- 4-hydroxycyclo- hexyl]-7H-pyrrolo- [2,3-d]pyrimidin- 5-yl]phenyl)meth- yl]piperazine-1- carboxylate | 561.50 | (400 MHz, CD₃OD) δ 8.72 (s, 1H), 7.61 (d, J = 8.2 Hz, 2H), 7.44-7.37 (m, 3H), 5.51 (s, 1H), 4.68-4.43 (m, 1H), 3.79-3.68 (m, 1H), 3.58 (s, 2H), 3.56-3.35 (m, 5H), 2.56-2.32 (m, 4H), 2.14 (d, J = 12.4 Hz, 2H), 2.10-1.97 (m, 4H), 1.59- 1.46 (m, 2H), 1.47 (s, 9H), 1.22-1.15 (m, 1H), 0.60- 0.51 (m, 2H), 0.37-0.29 (m, 2H). |
| 81-7 | 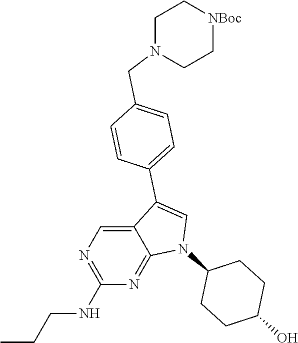 [SMILES: CCCNc1ncc2c(-c3ccc(CN4CCN(C(=O)OC(C)(C)C)CC4)cc3)cn([C@H]3CC[C@H](O)CC3)c2n1] | tert-butyl-4-([4-[2- (propylamino)-7- [trans-4-hydroxy- cyclohexyl]-7H- pyrrolo[2,3-d]- pyrimidin-5-yl]- phenyl]methyl)- piperazine-1- carboxylate | 549.50 | (400 MHz, CD₃OD) δ 8.70 (s, 1H), 7.59 (d, J = 8.0 Hz, 2H), 7.41-7.34 (m, 3H), 5.51 (s, 1H), 4.59-4.46 (m, 1H), 3.79-3.65 (m, 1H), 3.54 (s, 2H), 3.52-3.36 (m, 5H), 2.49-2.36 (m, 4H), 2.17-2.09 (m, 2H), 2.08- 1.98 (m, 4H), 1.79-1.62 (m, 2H), 1.60-1.49 (m, 2H), 1.46 (s, 9H), 1.03 (t, J = 7.4 Hz, 3H). |
| 81-8 | 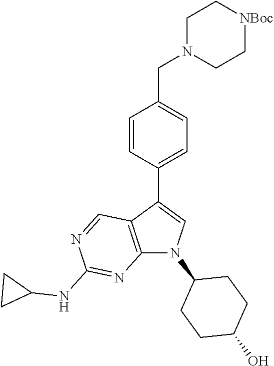 [SMILES: CC(C)(C)OC(=O)N1CCN(Cc2ccc(-c3cn([C@H]4CC[C@H](O)CC4)c4nc(NC5CC5)ncc34)cc2)CC1] | tert-butyl-4-([4-[2- (cyclopropyl- amino)-7-[trans- 4-hydroxycyclo- hexyl]-7H-pyrrolo- [2,3-d]pyrimidin- 5-yl]phenyl]meth- yl)piperazine-1- carboxylate | 547.40 | (400 MHz, CD₃OD) δ 8.75 (s, 1H), 7.61 (d, J = 7.9 Hz, 2H), 7.46-7.37 (m, 3H), 4.60-4.56 (m, 1H), 3.78- 3.65 (m, 1H), 3.57 (s, 2H), 3.49-3.41 (m, 3H), 2.82- 3.72 (m, 1H), 2.50-2.32 (m, 4H), 2.13 (d, J = 12.2 Hz, 2H), 2.10-2.02 (m, 4H), 1.59-1.49 (m, 2H), 1.47 (s, 9H), 0.89-0.78 (m, 2H), 0.61-0.53 (m, 2H). |
| 81-9 | 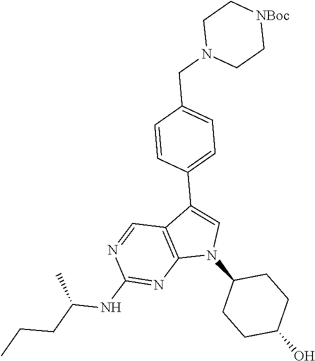 [SMILES: CCC[C@H](C)Nc1ncc2c(-c3ccc(CN4CCN(C(=O)OC(C)(C)C)CC4)cc3)cn([C@H]3CC[C@H](O)CC3)c2n1] | tert-butyl-4-[(4-[2- [(2S)-pentan-2- ylamino]-7-[trans- 4-hydroxycyclo- hexyl]pyrrolo[2,3- d]pyrimidin-5-yl]- phenyl)methyl]- piperazine-1- carboxylate | 577.50 | (400 MHz, CD₃OD) δ 8.71 (s, 1H), 7.65-7.57 (m, 2H), 7.42-7.36 (m, 3H), 4.58- 4.50 (m, 1H), 4.19-4.06 (m, 1H), 3.79-3.62 (m, 1H), 3.56 (s, 2H), 3.54-3.36 (m, 4H), 2.55-2.36 (m, 4H), 2.13 (d, J = 12.1 Hz, 2H), 2.09-1.95 (m, 4H), 1.69- 1.52 (m, 1H), 1.53 (t, J = 10.2 Hz, 3H), 1.47 (s, 9H), 1.36-1.15 (m, 3H), 1.09- 0.92 (m, 4H). |
| 81-10 | 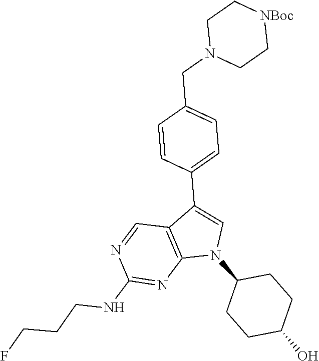 [SMILES: CC(C)(C)OC(=O)N1CCN(Cc2ccc(-c3cn([C@H]4CC[C@H](O)CC4)c4nc(NCCCF)ncc34)cc2)CC1] | tert-butyl-4-[(4-[2- [(3-fluoropropyl)- amino]-7-[trans-4- hydroxycyclo- hexyl]-7H-pyrrolo- [2,3-d]pyrimidin-5- yl]phenyl)methyl]- piperazine-1- carboxylate | 567.35 | (400 MHz, DMSO-d₆) δ 8.85 (s, 1H), 7.66-7.55 (m, 3H), 7.32 (d, J = 7.9 Hz, 2H), 6.96 (t, J = 5.7 Hz, 1H), 4.71 (d, J = 4.5 Hz, 1H), 4.62 (t, J = 5.9 Hz, 1H), 4.53-4.38 (m, 2H), 3.59-3.38 (m, 5H), 2.39- 2.25 (m, 4H), 2.10-1.89 (m, 8H), 1.49-1.25 (m, 11H). |
| 81-11 | 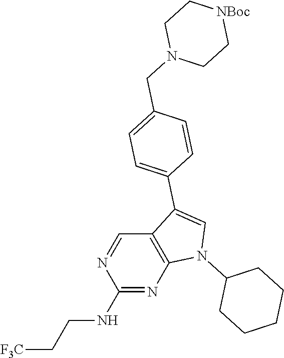 [SMILES: CC(C)(C)OC(=O)N1CCN(Cc2ccc(-c3cn(C4CCCCC4)c4nc(NCCC(F)(F)F)ncc34)cc2)CC1] | tert-butyl-4-[(4- [7-cyclohexyl-2- [(3,3,3-trifluoro- propyl)amino]- 7H-pyrrolo[2,3-d]- pyrimidin-5-yl]- phenyl)methyl]- piperazine-1- carboxylate | 587.30 | (400 MHz, DMSO-d₆) δ 8.89 (s, 1H), 7.69-7.63 (m, 3H), 7.33 (d, J = 7.9 Hz, 2H), 7.05 (t, J = 5.8 Hz, 1H), 4.44 (t, J = 10.8 Hz, 1H), 3.61-3.44 (m, 4H), 2.69-2.52 (m, 2H), 2.44-2.21 (m, 4H), 1.99-1.80 (m, 6H), 1.72 (d, J = 12.3 Hz, 1H), 1.55-1.32 (m, 12H), 1.30-1.14 (m, 4H). |
| 81-12 | 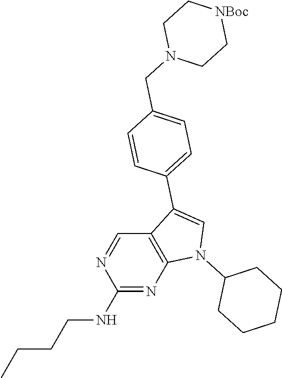 [SMILES: CCCCNc1ncc2c(-c3ccc(CN4CCN(C(=O)OC(C)(C)C)CC4)cc3)cn(C3CCCCC3)c2n1] | tert-butyl-4-([4-[2- (butylamino)-7- cyclohexyl-7H- pyrrolo[2,3-d]- pyrimidin-5-yl]- phenyl]methyl)- piperazine-1- carboxylate | 547.50 | (400 MHz, CDCl₃) δ 8.79 (s, 1H), 7.74-7.66 (m, 1H), 7.59-7.47 (m, 4H), 7.39 (d, J = 7.8 Hz, 2H), 7.13 (s, 1H), 3.60-3.43 (m, 8H), 2.45 (s, 4H), 2.15-2.05 (m, 3H), 1.95 (d, J = 13.2 Hz, 2H), 1.85- 1.61 (m, 5H), 1.54-1.34 (m, 13H), 1.34-1.24 (m, 3H). |
| 81-13 | 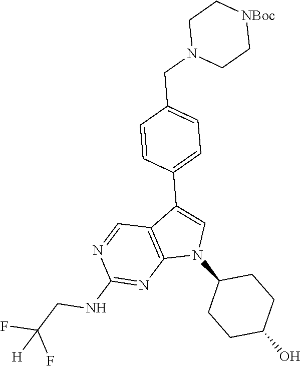 [SMILES: CC(C)(C)OC(=O)N1CCN(Cc2ccc(-c3cn([C@H]4CC[C@H](O)CC4)c4nc(NCC(F)F)ncc34)cc2)CC1] | tert-butyl-4-[(4-[2- [(2,2-difluoro- ethyl)amino]-7- [trans-4-hydroxy- cyclohexyl]-7H- pyrrolo[2,3-d]- pyrimidin-5-yl]- phenyl)methyl]- piperazine-1- carboxylate | 571.25 | (400 MHz, CD₃OD) δ 8.78 (s, 1H), 7.62 (d, J = 8.1 Hz, 2H), 7.46 (s, 1H), 7.40 (d, J = 8.0 Hz, 2H), 6.09-6.00 (m, 1H), 4.65-4.51 (m, 1H), 3.89-2.75 (m, 2H), 3.78- 3.67 (m, 1H), 3.57 (s, 2H), 3.49-3.42 (m, 4H), 2.56- 2.45 (m, 4H), 2.16 (s, 1H), 2.20-2.04 (m, 5H), 2.03 (d, J = 4.5 Hz, 1H), 1.57 (s, 1H), 1.57-1.32 (m, 8H). |
| 81-14 | 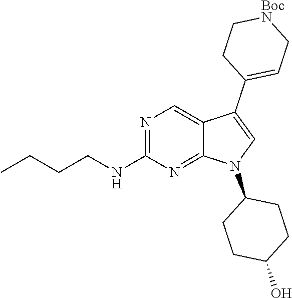 [SMILES: CCCCNc1ncc2c(C3=CCN(C(=O)OC(C)(C)C)CC3)cn([C@H]3CC[C@H](O)CC3)c2n1] | tert-butyl-4-[2- (butylamino)-7- [trans-4-hydroxy- cyclohexyl]-7H- pyrrolo[2,3-d]- pyrimidin-5-yl]- 1,2,3,6-tetra- hydropyridine- 1-carboxylate | 470.30 | (400 MHz, DMSO-d₆) δ 8.75 (s, 1H), 7.23 (s, 1H), 6.80 (t, J = 5.8 Hz, 1H), 6.11 (s, 1H), 4.69 (d, J = 4.4 Hz, 1H), 4.34 (d, J = 12.1 Hz, 1H), 4.00 (s, 2H), 3.59-3.42 (m, 3H), 3.30 (q, J = 6.5 Hz, 2H), 2.42 (s, 2H), 2.00-1.76 (m, 6H), 1.56- 1.52 (m, 2H), 1.53-1.42 (m, 13H), 0.92 (t, J = 7.3 Hz,3H). |
| 81-15 | 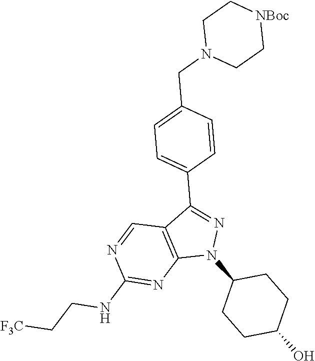 [SMILES: CC(C)(C)OC(=O)N1CCN(Cc2ccc(-c3nn([C@H]4CC[C@H](O)CC4)c4nc(NCCC(F)(F)F)ncc34)cc2)CC1] | tert-butyl-4-[(4-[1- [trans-4-hydroxy- cyclohexyl]-6- [(3,3,3-trifluoro- propyl)amino]-1H- pyrazolo[3,4-d]- pyrimidin-3-yl]- phenyl)methyl]- piperazine-1- carboxylate | 604.30 | (400 MHz, DMSO-d₆) δ 9.15 (s, 1H), 7.93 (d, J = 8.0 Hz, 2H), 7.65-7.61 (m, 1H), 7.43 (d, J = 8.1 Hz, 2H), 4.71 (d, J = 4.4 Hz, 1H), 4.51 (s, 1H), 3.69-3.51 (m, 3H), 3.54 (s, 2H), 3.45-3.21 (m, 6H), 2.62 (s, 2H), 2.44-2.21 (m, 4H), 2.07 (s, 2H), 2.10-1.90 (m, 4H), 1.40 (s, 9H). |
| 81-16 | 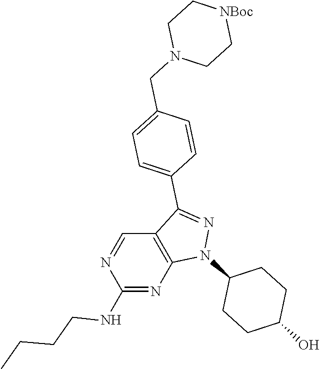 [SMILES: CCCCNc1ncc2c(-c3ccc(CN4CCN(C(=O)OC(C)(C)C)CC4)cc3)nn([C@H]3CC[C@H](O)CC3)c2n1] | tert-butyl-4-([4-[2- (butylamino)-7- [trans-4-hydroxy- cyclohexyl]-7H- pyrrolo[2,3-d]- pyrimidin-5-yl]- phenyl]methyl)- piperazine-1- carboxylate | 564.40 | (400 MHz, DMSO-d₆) δ 12.27 (s, 1H), 9.77 (s, 1H), 9.32 (s, 1H), 8.07 (d, J = 7.9 Hz, 2H), 7.81 (d, J = 8.0 Hz, 2H), 4.54 (s, 1H), 4.48 (s, 2H), 3.70- 3.64 (m, 1H), 3.67-3.55 (m, 4H), 3.55-3.45 (m, 5H), 3.41 (t, J = 7.5 Hz, 3H), 2.11-1.92 (m, 5H), 1.59 (d, J = 8.1 Hz, 2H), 1.45-1.32 (m, 4H), 0.95 (t, J = 7.4 Hz, 3H). |
| 81-17 | 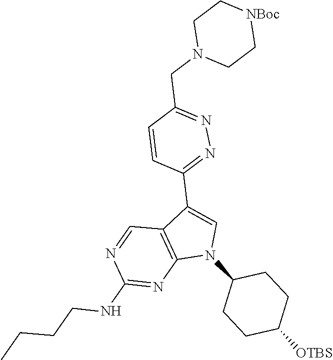 [SMILES: CCCCNc1ncc2c(-c3ccc(CN4CCN(C(=O)OC(C)(C)C)CC4)nn3)cn([C@H]3CC[C@H](O[Si](C)(C)C(C)(C)C)CC3)c2n1] | tert-butyl-4-([6-[2- (butylamino)-7- [trans-4-[(tert- butyldimethylsilyl)- oxy]cyclohexyl]- 7H-pyrrolo[2,3-d]- pyrimidin-5-yl]- pyridazin-3-yl]- methyl)piperazine- 1-carboxylate | 679.60 | (400 MHz, CD₃OD) δ 9.24 (d, J = 6.2 Hz, 1H), 8.04 (d, J = 9.0 Hz, 1H), 7.95 (s, 1H), 7.82-7.75 (m, 1H), 7.79-7.62 (m, 1H), 4.29 (t, J = 6.6 Hz, 2H), 3.94-3.75 (m, 3H), 3.47 (s, 1H), 3.45 (s, 2H), 3.43 (s, 1H), 2.50 (s, 2H), 2.48 (d, J = 4.7 Hz, 2H), 2.08 (s, 3H), 2.05 (s, 2H), 1.98 (s, 2H), 1.79-1.61 (m, 7H), 1.28 (s, 2H), 1.35-1.12 (m, 4H), 1.21- 0.92 (m, 8H), 0.93 (s, 9H), 0.12 (s, 6H). |

### Step 2: tert-butyl 4-[[trans-4-[7-[trans-4-hydroxycyclohexyl]-2-[(3,3,3-trifluoropropyl)amino]pyrrolo[2,3-d]pyrimidin-5-yl]cyclohexyl]methyl]piperazine-1-carboxylate and tert-butyl 4-[[cis-4-[7-[trans-4-hydroxycyclohexyl]-2-[(3,3,3-trifluoropropyl)amino]pyrrolo[2,3-d]pyrimidin-5-yl]cyclohexyl]methyl]piperazine-1-carboxylate

To a stirred solution of tert-butyl 4-[(4-[7-[trans-4-hydroxycyclohexyl]-2-[(3,3,3-trifluoropropyl)amino]pyrrolo[2,3-d]pyrimidin-5-yl]cyclohex-3-en-1-yl)methyl]piperazine-1-carboxylate (300 mg, 0.49 mmol) in THF (30.0 mL) was added 10% palladium on activated carbon (30 mg) at room temperature. The resulting mixture was purged with hydrogen three times and stirred for 16 h at 40° C. under hydrogen atmosphere. The resulting mixture was filtered. The filter cake was washed with EtOAc (3×10.0 mL). The filtrate was concentrated under reduced pressure. The residue was purified by Prep-HPLC with the following conditions (Column: XBridge Prep C18 OBD Column, 19×150 mm 5 um; Mobile Phase A: water (10 mmol/L NH₄HCO₃), Mobile Phase B: ACN; Flow rate: 25 mL/min; Gradient: 50% B to 60% B in 12 min; Detector: UV 220/254 nm;) to afford tert-butyl 4-[[trans-4-[7-[trans-4-hydroxycyclohexyl]-2-[(3,3,3-trifluoropropyl)amino]pyrrolo[2,3-d]pyrimidin-5-yl]cyclohexyl]methyl]piperazine-1-carboxylate (30 mg, 10%) as a white solid and tert-butyl 4-[[cis-4-[7-[trans-4-hydroxycyclohexyl]-2-[(3,3,3-trifluoropropyl)amino]pyrrolo[2,3-d]pyrimidin-5-yl]cyclohexyl]methyl]piperazine-1-carboxylate (50 mg, 17%) as a white solid.

82: ¹H NMR (400 MHz, DMSO-d₆) δ 8.56 (s, 1H), 6.90-6.79 (m, 2H), 4.65 (d, J=4.4 Hz, 1H), 4.37-4.25 (m, 1H), 3.59-3.51 (m, 3H), 3.30 (s, 3H), 2.68-2.53 (m, 3H), 2.48-2.33 (m, 4H), 2.13 (d, J=7.1 Hz, 2H), 2.02-1.76 (m, 12H), 1.57 (d, J=8.6 Hz, 1H), 1.51-1.42 (m, 2H), 1.40 (s, 9H), 1.31-1.24 (m, 2H), 1.01 (q, J=11.9 Hz, 2H). LC/MS (ESI, m/z): [(M+1)]⁺=609.35

83: ¹H NMR (400 MHz, DMSO-d₆) δ 8.53 (s, 1H), 6.93 (s, 1H), 6.83 (t, J=5.9 Hz, 1H), 4.65 (d, J=4.5 Hz, 1H), 4.32 (s, 1H), 3.53 (q, J=6.7 Hz, 3H), 3.32 (s, 2H), 2.80 (s, 1H), 2.68-2.54 (m, 2H), 2.59-2.44 (m, 4H), 2.38-2.19 (m, 6H), 2.02-1.78 (m, 7H), 1.72-1.62 (m, 4H), 1.66-1.49 (m, 4H), 1.40 (s, 9H). LC/MS (ESI, m/z): [(M+1)]⁺=609.35

The following intermediates in Table 7 were synthesized according to the above procedure:

TABLE 7

| Inter- | | | MS: | |
|---|---|---|---|---|
| medi- | | | [(M + | |
| ate | Structure | Name | 1)]⁺ | ¹H NMR |
| 84 | 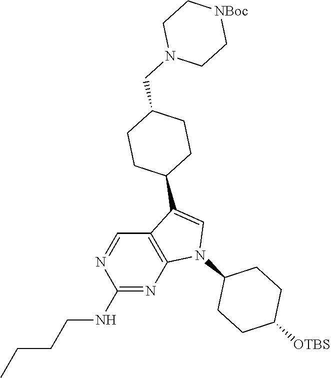 [SMILES: CCCCNc1ncc2c(n1)n([C@H]1CC[C@H](O[Si](C)(C)C(C)(C)C)CC1)cc2[C@H]1CC[C@H](CN2CCN(C(=O)OC(C)(C)C)CC2)CC1] | trans-tert-butyl-4- [[4-[2-(butylamino)- 7-[trans-4-[(tert- butyldimethylsilyl) oxy]cyclohexyl]-7H- pyrrolo[2,3- d]pyrimidin-5- yl]cyclohexyl]methyl] piperazine-1- carboxylate | 683.50 | (400 MHz, CDCl₃) δ 8.47 (s, 1H), 6.57 (d, J = 0.9 Hz, 1H), 5.23 (s, 1H), 4.43 (tt, J = 12.0, 3.6 Hz, 1H), 3.77-3.64 (m, 1H), 3.55-3.36 (m, 6H), 2.70-2.57 (m, 1H), 2.43-2.29 (m, 4H), 2.20 (d, J = 7.1 Hz, 2H), 2.13-1.89 (m, 9H), 1.87-1.69 (m, 3H), 1.69-1.52 (m, 6H), 1.48 (s, 9H), 1.32-1.23 (m, 4H), 1.18-1.01 (m, 2H), 0.92 (s, 9H), 0.10 (s, 6H). |
| 85 | 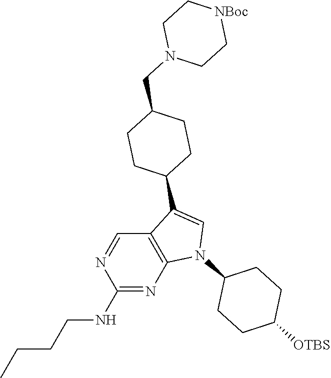 [SMILES: CCCCNc1ncc2c(n1)n([C@H]1CC[C@H](O[Si](C)(C)C(C)(C)C)CC1)cc2[C@H]1CC[C@@H](CN2CCN(C(=O)OC(C)(C)C)CC2)CC1] | cis-tert-butyl-4-[[4- [2-(butylamino)-7- [trans-4-[(tert- butyldimethylsilyl) oxy]cyclohexyl]-1H- pyrrolo[2,3- d]pyrimidin-5- yl]cyclohexyl]methyl] piperazine-1- carboxylate | 683.50 | (400 MHz, CDCl₃) δ 8.46 (s, 1H), 6.61 (d, J = 1.1 Hz, 1H), 5.31 (s, 1H), 4.49-4.35 (m, 1H), 3.79-3.70 (m, 1H), 3.54-3.41 (m, 6H), 2.46- 2.31 (m, 4H), 2.29 (d, J = 7.5 Hz, 2H), 2.07-1.97 (m, 4H), 1.89-1.71 (m, 7H), 1.66-1.52 (m, 8H), 1.48 (s, 9H), 1.31-1.24 (m, 4H), 0.97 (s, 2H), 0.92 (s, 9H), 0.10 (s, 6H). |
| 86 | 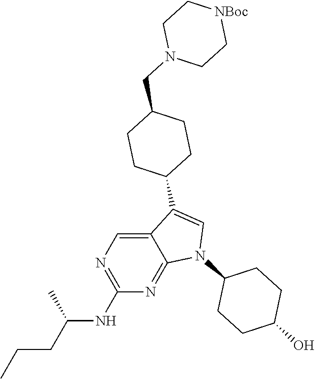 [SMILES: CCC[C@H](C)Nc1ncc2c(n1)n([C@H]1CC[C@H](O)CC1)cc2[C@H]1CC[C@H](CN2CCN(C(=O)OC(C)(C)C)CC2)CC1] | trans-tert-butyl-4- [[4-[2-[(2S)-pentan- 2-ylamino]-7-[trans- 4- hydroxycyclohexyl] pyrrolo[2,3- d]pyrimidin-5- yl]cyclohexyl]methyl] piperazine-1- carboxylate | 583.50 | crude to next step without further purification |
| 87 | 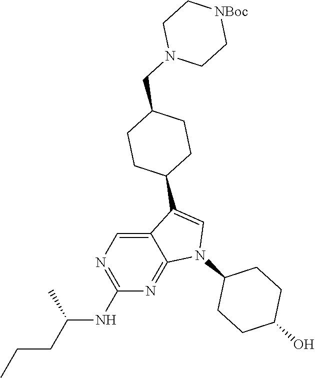 [SMILES: CCC[C@H](C)Nc1ncc2c(n1)n([C@H]1CC[C@H](O)CC1)cc2[C@H]1CC[C@@H](CN2CCN(C(=O)OC(C)(C)C)CC2)CC1] | cis-tert-butyl-4-[[4- [2-[(2S)-pentan-2- ylamino]-7-[trans-4- hydroxycyclohexyl] pyrrolo[2,3- d]pyrimidin-5- yl]cyclohexyl]methyl] piperazine-1- carboxylate | 583.50 | crude to next step without further purification |

### Step 4: trans-4-[5-[trans-4-(piperazin-1-ylmethyl)cyclohexyl]-2-[(3,3,3-trifluoropropyl)amino]pyrrolo[2,3-d]pyrimidin-7-yl]cyclohexan-1-ol hydrochloride

To a stirred solution of tert-butyl 4-[[trans-4-[7-[trans-4-hydroxycyclohexyl]-2-[(3,3,3-trifluoropropyl)amino]pyrrolo[2,3-d]pyrimidin-5-yl]cyclohexyl]methyl]piperazine-1-carboxylate (30.0 mg, 0.05 mmol) in 1,4-dioxane (3.00 mL) was added HCl (4 Min 1,4-dioxane, 3.00 mL) dropwise at room temperature under nitrogen atmosphere. The resulting mixture was stirred for 2 h at room temperature. The resulting mixture was concentrated under reduced pressure to afford trans-4-[5-[trans-4-(piperazin-1-ylmethyl)cyclohexyl]-2-[(3,3,3-trifluoropropyl)amino]pyrrolo[2,3-d]pyrimidin-7-yl]cyclohexan-1-ol hydrochloride (25.0 mg, 93%) as a light yellow solid. ¹H NMR (400 MHz, CD₃OD) δ 8.74 (s, 1H), 7.33 (s, 1H), 4.64-4.45 (m, 2H), 3.89-3.78 (m, 4H), 2.80 (t, J=11.4 Hz, 1H), 2.69-2.59 (m, 3H), 2.19-1.97 (m, 14H), 1.69-1.46 (m, 7H), 1.43-1.25 (m, 4H). LC/MS (ESI, m/z): [(M+1)]⁺=509.35

The following intermediates in Table 8 were synthesized according to the above procedure:

TABLE 8

| Inter- | | | MS: | |
|---|---|---|---|---|
| medi- | | | [(M + | |
| ate | Structure | Name | 1)]⁺ | ¹H NMR |
| O2 | 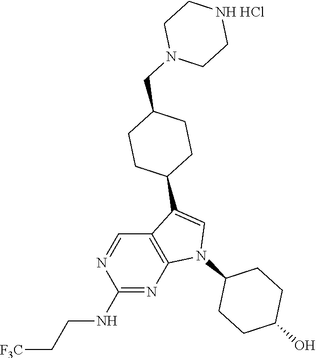 | trans-4-[5-[4-(cis- piperazin-1- ylmethyl)cyclohexyl]- 2-[(3,3,3- trifluoropropyl)amino] pyrrolo[2,3- d]pyrimidin-7- yl]cyclohexan-1-ol hydrochloride | 509.30 | (400 MHz, DMSO-d₆) δ 8.92 (s, 1H), 8.03 (s, 1H), 7.38 (s, 1H), 4.47-4.31 (m, 2H), 3.69 (s, 7H), 3.51 (d, J = 9.9 Hz, 4H), 3.27- 3.13 (m, 2H), 2.85 (s, 1H), 2.69- 2.56 (m, 2H), 2.20 (s, 1H), 2.03- 1.81 (m, 6H), 1.79-1.62 (m, 8H), 1.39-1.25 (m, 2H), 1.24 (s, 1H). |
| O3 | 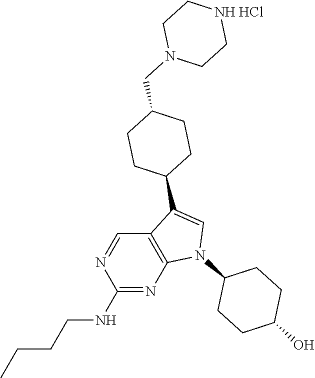 | trans-4-[2- (butylamino)-5- [trans-4-[(piperazin- 1- yl)methyl]cyclohexyl]- 7H-pyrrolo[2,3- d]pyrimidin-7- yl]cyclohexan-1-ol hydrochloride | 469.50 | (400 MHz, DMSO-d₆) δ 8.31 (s, 1H), 7.05 (s, 1H), 4.29-4.20 (m, 1H), 3.62-3.49 (m, 7H), 3.29 (t, J = 7.1 Hz, 2H), 3.13 (d, J = 7.0 Hz, 2H), 2.59-2.50 (m, 1H), 2.03- 1.58 (m, 12H), 1.54-1.07 (m, 11H), 0.80 (t, J = 7.4 Hz, 3H). |
| O4 | 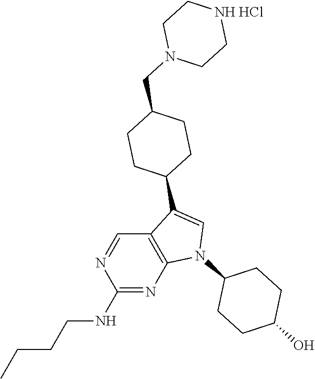 | trans-4-[2- (butylamino)-5-[cis- 4-[(piperazin-1- yl)methyl]cyclohexyl]- 7H-pyrrolo[2,3- d]pyrimidin-7- yl]cyclohexan-1-ol hydrochloride | 469.50 | (400 MHz, DMSO-d₆) δ 8.31 (s, 1H), 7.05 (s, 1H), 4.29-4.20 (m, 1H), 3.62-3.49 (m, 7H), 3.29 (t, J = 7.1 Hz, 2H),3.13 (d, J = 7.0 Hz, 2H), 2.59-2.50 (m, 1H), 2.03- 1.58 (m, 12H), 1.54-1.07 (m, 11H), 0.80 (t, J = 7.4 Hz, 3H). |
| O5 | 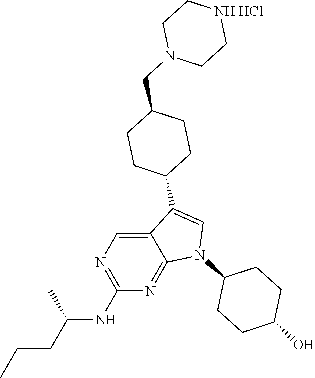 | trans-4-[2-[(2S)- pentan-2-ylamino]-5- [trans-4-(piperazin-1- ylmethyl)cyclohexyl] pyrrolo[2,3- d]pyrimidin-7- yl]cyclohexan-1-ol hydrochloride | 483.50 | (400 MHz, DMSO-d₆) δ 13.86 (s, 1H), 11.49 (s, 1H), 9.87 (d, J = 70.3 Hz, 2H), 8.89 (s, 1H), 8.04 (s, 1H), 7.76-7.64 (m, 1H), 7.35 (s, 1H), 4.39-4.31 (m, 1H), 4.13- 3.81 (m, 5H), 3.76-3.47 (m, 8H), 3.32 (d, J = 14.1 Hz, 2H), 3.14- 2.93 (m, 2H), 2.71-2.59 (m, 1H), 2.11-1.75 (m, 12H), 1.67-1.58 (m, 2H), 1.53-1.44 (m, 2H), 1.48- 1.30 (m, 7H), 1.29-1.15 (m, 4H), 1.14-1.03 (m, 2H), 0.99-0.85 (m, 6H). |
| O6 | 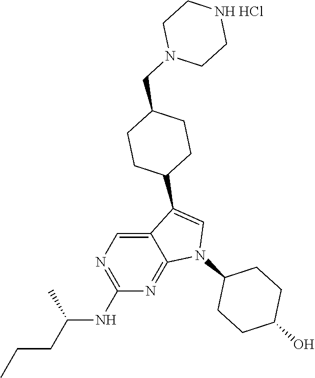 | trans-4-[2-[(2S)- pentan-2-ylamino]-5- [cis-4-(piperazin-1- ylmethyl)cyclohexyl] pyrrolo[2,3- d]pyrimidin-7- yl]cyclohexan-1-ol hydrochloride | 483.50 | (400 MHz, DMSO-d₆) δ 13.90 (s, 1H), 11.57 (s, 1H), 10.11 (s, 1H), 9.89 (s, 1H), 8.90 (s, 1H), 8.14 (d, J = 7.5 Hz, 1H), 7.36 (s, 1H), 4.36 (m, 1H), 4.07 (m, 1H), 3.79-3.68 (m, 2H), 3.66-3.56 (m, 3H), 3.58- 3.41 (m, 3H), 3.32 (s, 2H), 3.23 (d, J = 6.5 Hz, 2H), 2.81 (s, 1H), 2.20 (s, 1H), 1.99-1.89 (m, 4H), 1.89-1.79 (m, 2H), 1.78-1.63 (m, 7H), 1.63-1.45 (m, 2H), 1.46- 1.33 (m, 4H), 1.22 (d, J = 6.5 Hz, 3H), 0.92 (t, J = 7.2 Hz, 3H). |
| O7 | 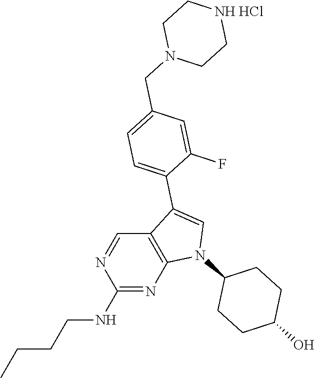 | trans-4-[2- (butylamino)-5-[2- fluoro-4-[(piperazin- 1-yl)methyl]phenyl]- 7H-pyrrolo[2,3- d]pyrimidin-7- yl]cyclohexan-1-ol hydrochloride | 481.30 | (400 MHz, CD₃OD) δ 8.71 (s, 1H), 7.96 (s, 1H), 7.84 (t, J = 7.9 Hz, 1H), 7.71-7.59 (m, 6H), 4.69- 4.60 (m, 1H), 4.59-4.50 (m, 2H), 3.75 (s, 1H), 3.66-3.54 (m, 10H), 2.18-2.12 (m, 6H), 1.74 (p, J = 7.3 Hz, 2H), 1.59-1.42 (m, 4H), 1.05 (t, J = 7.3 Hz, 3H). |
| O8 | 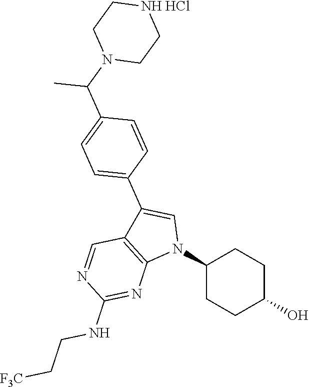 | trans-4-(5-[4-[1- (piperazin-1- yl)ethyl]phenyl]-2- [(3,3,3- trifluoropropyl)amino]- 7H-pyrrolo[2,3- d]pyrimidin-7- yl)cyclohexan-1-ol hydrochloride | 517.35 | (400 MHz, CD₃OD) δ 8.97-8.92 (m, 1H), 8.03 (d, J = 1.5 Hz, 1H), 7.88-7.76 (m, 4H), 4.69-4.55 (m, 2H), 3.88 (t, J = 7.0 Hz, 2H), 3.81-3.70 (m, 3H), 3.69-3.35 (m, 7H), 2.72-2.62 (m, 2H), 2.17- 2.08 (m, 5H), 1.89 (t, J = 5.2 Hz, 3H), 1.59-1.45 (m, 2H). |
| O9^{(a)} | 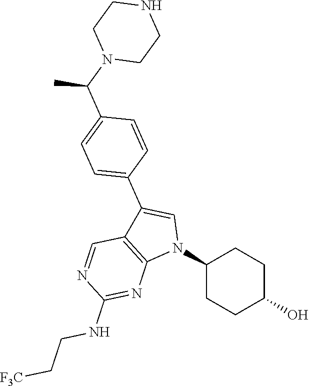 [SMILES: C[C@H](c1ccc(-c2cn([C@H]3CC[C@H](O)CC3)c3nc(NCCC(F)(F)F)ncc23)cc1)N1CCNCC1] | trans-4-(5-[4-[(1R)-1- (piperazin-1- yl)ethyl]phenyl]-2- [(3,3,3- trifluoropropyl)amino] pyrrolo[2,3- d]pyrimidin-7- yl)cyclohexan-1-ol | 517.35 | (400 MHz, DMSO-d₆) δ 8.88 (s, 1H), 7.69-7.62 (m, 3H), 7.33 (d, J = 8.0 Hz, 2H), 7.07 (t, J = 5.9 Hz, 1H), 4.73 (d, J = 4.2 Hz, 1H), 4.49-4.32 (m, 1H), 3.59 (d, J = 6.6 Hz, 1H), 3.59-3.45 (m, 2H), 3.15-3.02 (m, 4H), 2.72-2.52 (m, 5H), 2.02-1.85 (m, 6H), 1.46- 1.32 (m, 5H), 1.28-1.10 (m, 3H). |
| O10^{(a)} | 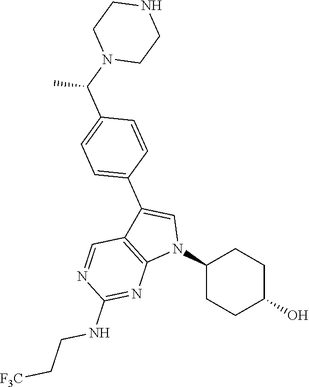 [SMILES: C[C@@H](c1ccc(-c2cn([C@H]3CC[C@H](O)CC3)c3nc(NCCC(F)(F)F)ncc23)cc1)N1CCNCC1] | trans-4-(5-[4-[(1S)-1- (piperazin-1- yl)ethyl]phenyl]-2- [(3,3,3- trifluoropropyl)amino] pyrrolo[2,3- d]pyrimidin-7- yl)cyclohexan-1-ol | 517.35 | (400 MHz, DMSO-d₆) δ 8.83 (s, 1H), 7.65 (d, J = 7.9 Hz, 2H), 7.59 (s, 1H), 7.33 (d, J = 7.8 Hz, 2H), 6.88 (s, 1H), 4.73 (d, J = 4.4 Hz, 1H), 4.44-4.37 (m, 1H), 3.55 (s, 1H), 3.44-3.30 (m, 10H), 3.15- 3.00 (m, 3H), 2.57 (s, 2H), 2.54- 2.48 (m, 10H), 2.09-1.90 (m, 3H), 1.89 (s, 2H), 1.66-1.42 (m, 2H), 1.40-1.29 (m, 7H), 1.29- 1.12 (m, 8H), 0.93 (t, J = 13 Hz, 3H). |
| O11 | 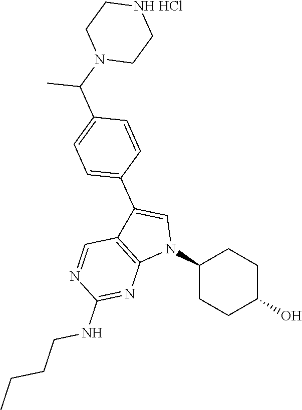 | trans-4-[2- (butylamino)-5-[4-[1- (piperazin-1- yl)ethyl]phenyl]-1H- pyrrolo[2,3- d]pyrimidin-7- yl]cyclohexan-1-ol hydrochloride | 477.30 | (400 MHz, CD₃OD) δ 8.85 (s, 1H), 7.99 (s, 1H), 7.87-7.72 (m, 4H), 4.65 (s, 2H), 4.12 (q, J = 7.1 Hz, 2H), 3.80-3.54 (m, 8H), 3.47 (s, 2H), 2.17 (d, J = 12.3 Hz, 2H), 2.19-2.04 (m, 4H), 2.03-1.95 (m, 3H), 1.91-1.82 (m, 3H), 1.74 (q, J = 7.4 Hz, 2H), 1.59-1.41 (m, 4H), 1.26 (t, J = 7.1 Hz, 3H), 1.05 (t, J = 7.3 Hz, 3H). |
| O12^{(a)} | 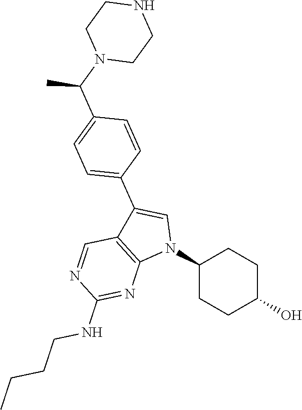 [SMILES: CCCCNc1ncc2c(-c3ccc([C@@H](C)N4CCNCC4)cc3)cn([C@H]3CC[C@H](O)CC3)c2n1] | trans-4-[2- (butylamino)-5-[4- [(1R)-1-(piperazin-1- yl)ethyl]phenyl]pyrrolo [2,3-d]pyrimidin-7- yl]cyclohexan-1-ol | 477.35 | (400 MHz, DMSO-d₆) δ 8.82 (s, 1H), 7.64 (d, J = 8.0 Hz, 2H), 7.58 (s, 1H), 7.32 (d, J = 7.9 Hz, 2H), 6.85 (t, J = 5.8 Hz, 1H), 4.72 (d, J = 4.4 Hz, 1H), 4.41 (s, 1H), 3.59-3.42 (m, 2H), 3.15-2.95 (m, 3H), 2.66-2.52 (m, 4H), 1.99- 1.90 (m, 3H), 1.89 (s, 2H), 1.59- 1.49 (m, 2H), 1.45-1.23 (m, 7H), 1.18 (d, J = 6.5 Hz, 2H), 0.92 (t, J = 7.3 Hz, 3H). |
| O13^{(a)} | 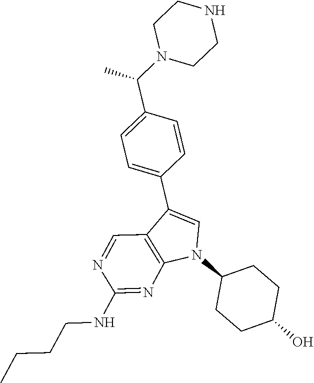 [SMILES: CCCCNc1ncc2c(-c3ccc([C@H](C)N4CCNCC4)cc3)cn([C@H]3CC[C@H](O)CC3)c2n1] | trans-4-[2- (butylamino)-5-[4- [(1S)-1-(piperazin-1- yl)ethyl]phenyl]pyrrolo [2,3-d]pyrimidin-7- yl]cyclohexan-1-ol | 477.35 | (400 MHz, DMSO-d₆) δ 8.88 (s, 1H), 7.70-7.61 (m, 3H), 7.33 (d, J = 8.0 Hz, 2H), 7.07 (t, J = 5.9 Hz, 1H), 4.73 (d, J = 4.2 Hz, 1H), 4.44 (d, J = 12.9 Hz, 1H), 3.67- 3.49 (m, 4H), 3.15-3.00 (m, 4H), 2.61-2.54 (m, 4H), 1.99-1.90 (m, 3H), 1.91 (s, 3H), 1.43-1.27 (m, 5H), 1.28-1.06 (m, 5H). |
| O14 | 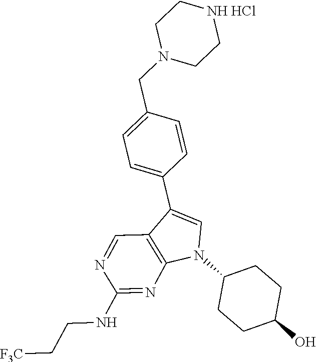 | trans-4-(5-[4- [(piperazin-1- yl)methyl]phenyl]-2- [(3,3,3- trifluoropropyl)amino]- 7H-pyrrolo[2,3- d]pyrimidin-7- yl)cyclohexan-1-ol hydrochloride | 503.30 | (400 MHz, DMSO-d₆) δ 12.15 (s, 1H), 9.62 (s, 2H), 9.10 (s, 1H), 8.10 (s, 1H), 7.83 (d, J = 7.9 Hz, 2H), 7.72 (d, J = 7.7 Hz, 2H), 4.55-4.36 (m, 3H), 3.79-3.66 (m, 4H), 3.66-3.42 (m, 4H), 3.25 (s, 2H), 2.76-2.62 (m, 2H), 2.04- 1.89 (m, 6H), 1.48-1.38 (m, 2H). |
| O15 | 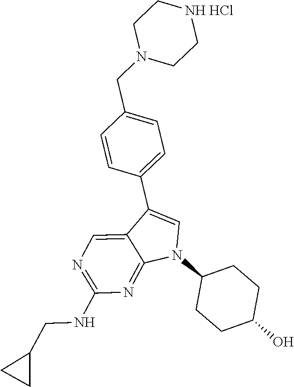 | trans-4-[2- [(cyclopropylmethyl) amino]-5-[4- [(piperazin-1- yl)methyl]phenyl]- 7H-pyrrolo[2,3- d]pyrimidin-7- yl]cyclohexan-1-ol hydrochloride | 461.35 | (400 MHz, CD₃OD) δ 8.86 (s, 1H), 8.00 (s, 1H), 7.86-7.75 (m, 4H), 4.69-4.60 (m, 1H), 4.57 (s, 2H), 3.78-3.60 (m, 6H), 3.44 (d, J = 7.1 Hz, 2H), 2.16 (d, J = 12.5 Hz, 2H), 2.19-2.00 (m, 3H), 1.56 (s, 1H), 0.92 (s, 2H), 0.90 (s, 2H), 0.64 (d, J = 7.7 Hz, 2H), 0.41 (d, J = 5.0 Hz, 2H). |
| O16 | 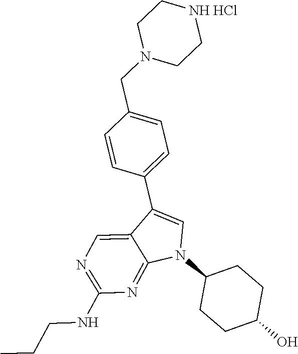 | trans-4-(5-[4- [(piperazin-1- yl)methyl]phenyl]-2- (propylamino)-7H- pyrrolo[2,3- d]pyrimidin-7- yl)cyclohexan-1-ol hydrochloride | 449.35 | (400 MHz, CD₃OD) δ 8.85 (s, 1H), 8.00 (s, 1H), 7.83 (d, J = 7.9 Hz, 2H), 7.78 (d, J = 8.1 Hz, 2H), 4.66-4.61 (m, 1H), 4.57 (s, 2H), 3.72-3.59 (m, 9H), 3.54 (t, J = 7.2 Hz, 2H), 2.17 (d, J = 12.1 Hz, 2H), 2.20-2.02 (m, 4H), 1.78 (q, J = 7.3 Hz, 2H), 1.57 (d, J = 9.6 Hz, 2H), 1.08 (t, J = 7.4 Hz, 3H). |
| O17 | 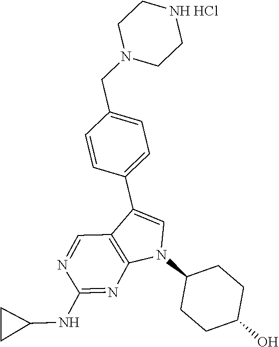 | trans-4-[2- (cyclopropylamino)- 5-[4-[(piperazin-1- yl)methyl]phenyl]- 7H-pyrrolo[2,3- d]pyrimidin-7- yl]cyclohexan-1-ol hydrochloride | 447.30 | (400 MHz, CD₃OD) δ 8.82 (s, 1H), 8.04 (s, 1H), 7.84 (d, J = 8.3 Hz, 2H), 7.78 (d, J = 8.1 Hz, 2H), 4.67-4.59 (m, 1H), 4.56 (s, 2H), 3.80-3.70 (m, 2H), 3.77-3.58 (m, 5H), 3.62 (s, 2H), 2.89-2.75 (m, 1H), 2.19-2.04 (m, 6H), 1.59- 1.45 (m, 2H), 1.09-0.99 (m, 2H), 0.81-0.73 (m, 2H). |
| O18 | 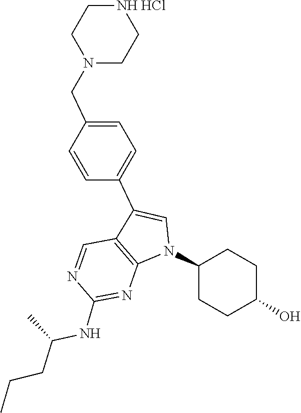 | trans-4-[2-[(2S)- pentan-2-ylamino]-5- [4-(piperazin-1- ylmethyl)phenyl]pyrrolo [2,3-d]pyrimidin- 7-yl]cyclohexan-1-ol hydrochloride | 477.30 | (400 MHz, CD₃OD) δ 8.83 (s, 1H), 7.98 (s, 1H), 7.82 (d, J = 8.0 Hz, 2H), 7.76 (d, J = 8.6 Hz, 2H), 7.71-7.60 (m, 1H), 4.63 (q, J = 7.9 Hz, 1H), 4.51 (s, 2H), 4.23 (d, J = 6.3 Hz, 1H), 3.80-3.70 (m, 1H), 3.78-3.66 (m, 3H), 3.66- 3.56 (m, 3H), 2.23-2.01 (m, 6H), 1.80-1.48 (m, 7H), 1.46-1.25 (m, 4H), 1.09-0.95 (m, 4H). |
| O19 | 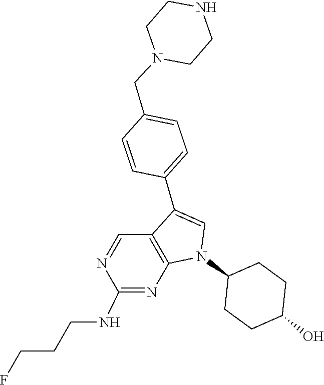 [SMILES: O[C@H]1CC[C@H](n2cc(-c3ccc(CN4CCNCC4)cc3)c3cnc(NCCCF)nc32)CC1] | trans-4-2-[(3- fluoropropyl)amino]- 5-[4-[(piperazin-1- yl)methyl]phenyl]- 7H-pyrrolo[2,3- d]pyrimidin-7- yl]cyclohexan-1-ol | 467.25 | (400 MHz, CD₃OD) δ 8.87 (s, 1H), 7.99 (s, 1H), 7.82 (d, J = 8.0 Hz, 1H), 7.73 (d, J = 7.9 Hz, 1H), 4.74-4.63 (m, 2H), 4.57 (t, J = 5.6 Hz, 1H), 4.45 (s, 1H), 3.79-3.65 (m, 3H), 3.68 (s, 2H), 3.62 (s, 5H), 3.53 (s, 2H), 2.19-2.04 (m, 7H), 1.63-1.49 (m, 2H), 1.37- 1.29 (m, 1H). |
| O20 | 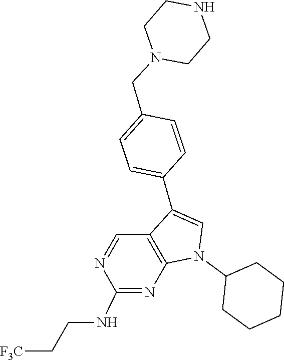 [SMILES: FC(F)(F)CCNc1ncc2c(-c3ccc(CN4CCNCC4)cc3)cn(C3CCCCC3)c2n1] | 7-cyclohexyl-5-[4- [(piperazin-1- yl)methyl]phenyl]-N- (3,3,3- trifluoropropyl)-7H- pyrrolo[2,3- d]pyrimidin-2-amine | 487.25 | (400 MHz, CD₃OD) δ 8.93 (s, 1H), 8.04 (s, 1H), 7.84 (d, J = 8.0 Hz, 2H), 7.76 (d, J = 8.1 Hz, 2H), 4.72-4.60 (m, 1H), 4.50 (s, 2H), 3.88 (t, J = 6.9Hz, 2H), 3.70-3.52 (m, 8H), 2.77-2.58 (m, 2H), 2.15- 1.79 (m, 8H), 1.67-1.51 (m, 2H), 1.49-1.32 (m, 1H). |
| O21 | 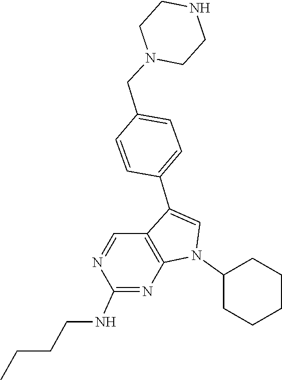 [SMILES: CCCCNc1ncc2c(-c3ccc(CN4CCNCC4)cc3)cn(C3CCCCC3)c2n1] | N-butyl-7-cyclohexyl- 5-[4-[(piperazin-1-yl) methyl]phenyl]-7H- pyrrolo[2,3-d] pyrimidin-2-amine | 447.3 | (400 MHz, CD₃OD) δ 8.85 (s, 1H), 8.00 (s, 1H), 7.86-7.73 (m, 4H), 4.68-4.55 (m, 3H), 3.72- 3.53 (m, 10H), 2.12-1.92 (m, 6H), 1.87-1.67 (m, 3H), 1.64- 1.32 (m, 5H), 1.05 (t, J = 13 Hz, 3H). |
| O22 | 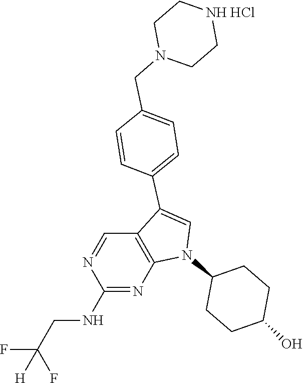 | trans-4-[2-[(2,2- difluoroethyl)amino]- 5-[4-[(piperazin-1- yl)methyl]phenyl]- 7H-pyrrolo[2,3- d]pyrimidin-7- yl]cyclohexan-1-ol hydrochloride | 471.40 | (400 MHz, CD₃OD) δ 8.96 (s, 1H), 8.06 (s, 1H), 7.85 (d, J = 8.1 Hz, 2H), 7.78 (d, J = 8.0 Hz, 2H), 6.23-6.12 (m, 1H), 4.68 (p, J = 8.6 Hz, 1H), 4.55 (s, 2H), 4.14- 4.01 (m, 2H), 3.83-3.71 (m, 2H), 3.70-3.66 (m, 4H), 3.62 (s, 1H), 2.30-1.91 (m, 7H), 1.68-1.52 (m, 2H). |
| O23 | 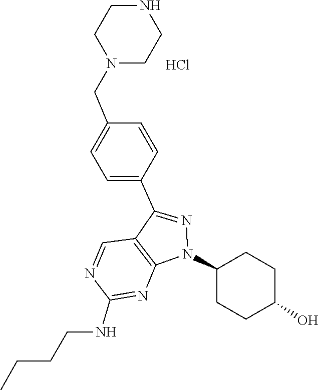 [SMILES: CCCCNc1ncc2c(-c3ccc(CN4CCNCC4)cc3)nn([C@H]3CC[C@H](O)CC3)c2n1 ; Cl] | trans-4-[6- (butylamino)-3-[4- [(piperazin-1- yl)methyl]phenyl]- 1H-pyrazolo[3,4- d]pyrimidin-1- yl]cyclohexan-1-ol hydrochloride | 464.25 | (400 MHz, DMSO-d₆) δ 12.27 (s, 1H), 9.77 (s, 1H), 9.32 (s, 1H), 8.07 (d, J = 7.9 Hz, 2H), 7.81 (d, J = 8.0 Hz, 2H), 4.54 (s, 1H), 4.48 (s, 2H), 3.70-3.64 (m, 1H), 3.57 (s, 4H), 3.55-3.45 (m, 5H), 3.41 (t, J = 7.5 Hz, 3H), 2.11-1.92 (m, 5H), 1.59 (d, J = 8.1 Hz, 2H), 1.49-1.31 (m, 4H), 0.95 (t, J = 7.4 Hz, 3H). |
| O24 | 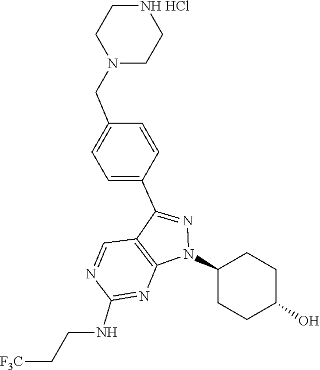 | trans-4-(3-[4- [(piperazin-1- yl)methyl]phenyl]-6- [(3,3,3- trifluoropropyl)amino]- 1H-pyrazolo[3,4- d]pyrimidin-1- yl)cyclohexan-1-ol hydrochloride | 504.20 | (400 MHz, DMSO-d₆) δ 8.08 (d, J = 7.9 Hz, 2H), 7.77 (d, J = 7.7 Hz, 2H), 7.65-7.62 (m, 1H), 7.53- 7.49 (m, 1H), 4.22 (s, 2H), 3.62 (s, 2H), 3.27 (s, 1H), 2.69-2.54 (m, 3H), 2.07 (s, 1H), 2.00 (s, 2H), 2.05-1.90 (m, 4H), 1.38 (t, J = 12.4 Hz, 2H), 1.18 (t, J = 7.1 Hz, 2H). |
| O25 | 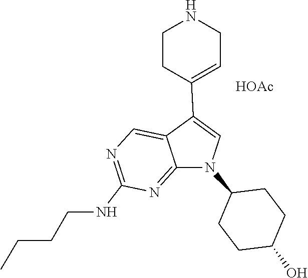 [SMILES: CCCCNc1ncc2c(C3=CCNCC3)cn([C@H]3CC[C@H](O)CC3)c2n1 ; "HOAc"] | trans-4-[2- (butylamino)-5- (1,2,3,6- tetrahydropyridin-4- yl)-7H-pyrrolo[2,3- d]pyrimidin-7- yl]cyclohexan-1-ol; acetic acid | 370.40 | (400 MHz, DMSO-d₆) δ 8.71 (d, J = 7.5 Hz, 1H), 7.18 (s, 1H), 6.78 (t, J = 5.8 Hz, 1H), 6.13 (s, 1H), 4.39-4.30 (m, 1H), 3.50 (d, J = 11.2 Hz, 2H), 3.39-3.31 (m, 2H), 2.94 (s, 2H), 2.34 (s, 2H), 2.00- 1.76 (m, 9H), 1.59-1.42 (m, 2H), 1.45-1.32 (m, 4H), 0.92 (t, J = 7.4 Hz, 3H). |
| O26 | 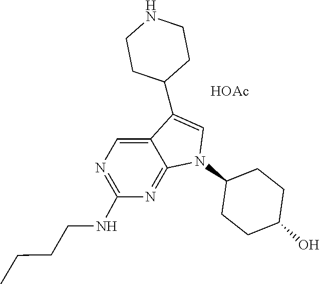 [SMILES: CCCCNc1ncc2c(C3CCNCC3)cn([C@H]3CC[C@H](O)CC3)c2n1 ; "HOAc"] | trans-4-[2- (butylamino)-5- (piperidin-4-yl)-7H- pyrrolo[2,3- d]pyrimidin-7- yl]cyclohexan-1-ol; acetic acid | 372.25 | (400 MHz, CD₃OD) δ 8.50 (s, 1H), 6.94 (s, 1H), 4.57-4.39 (m, 1H), 3.79-3.61 (m, 1H), 3.53- 3.40 (m, 4H), 3.22-3.04 (m, 3H), 2.23 (d, J = 14.3 Hz, 2H), 2.11 (d, J = 12.7 Hz, 2H), 2.03-1.86 (m, 11H), 1.69-1.55 (m, 2H), 1.57- 1.40 (m, 4H), 1.01 (t, J = 7.4 Hz, 3H). |
| O27 | 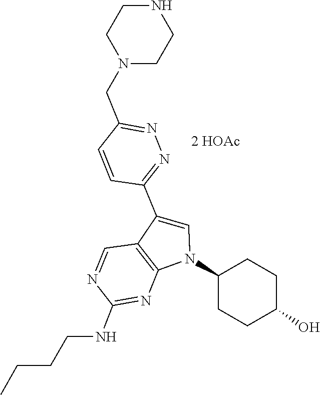 [SMILES: CCCCNc1ncc2c(-c3ccc(CN4CCNCC4)nn3)cn([C@H]3CC[C@H](O)CC3)c2n1] | trans-4-[2- (butylamino)-5-[6- [(piperazin-1- yl)methyl]pyridazin- 3-yl]-7H-pyrrolo[2,3- d]pyrimidin-7- yl]cyclohexan-1-ol; bis(acetic acid) | 465.40 | (400 MHz, CD₃OD) δ 9.25 (s, 1H), 8.08 (d, J = 8.9 Hz, 1H), 7.98 (s, 1H), 7.76 (d, J = 8.9 Hz, 1H), 4.59 (s, 1H), 3.92 (s, 2H), 3.74 (s, 1H), 3.47 (t, J = 7.1 Hz, 2H), 3.29-3.15 (m, 4H), 2.90- 2.72 (m, 4H), 2.21-2.00 (m, 6H), 1.77-1.56 (m, 2H), 1.59-1.44 (m, 4H), 1.02 (t, J = 7.4 Hz, 3H). |

^{(a)}:The mixture was separated in this step with the following conditions: Column: CHIRALPAK IG, 2 x 25 cm, 5 um; Mobile Phase A: Hex (0.2% isopropylamine), Mobile Phase B: EtOH; Flow rate: 20 mL/min; Gradient: 10% B to 10% B in 18 min; detector: UV 254/220 nm; RT1: 10.85 min; RT2: 13.14 min.

### INTERMEDIATE O28: tert-butyl 4-[(4-[7-[trans-4-hydroxycyclohexyl]-2-[[(2S)-4,4,4-trifluorobutan-2-yl]amino]-7H-pyrrolo[2,3-d]pyrimidin-5-yl]phenyl)methyl]piperazine-1-carboxylate hydrochloride and Intermediate O29: tert-butyl 4-[(4-[7-[trans-4-hydroxycyclohexyl]-2-[[(2R)-4,4,4-trifluorobutan-2-yl]amino]-7H-pyrrolo[2,3-d]pyrimidin-5-yl]phenyl)methyl]piperazine-1-carboxylate hydrochloride

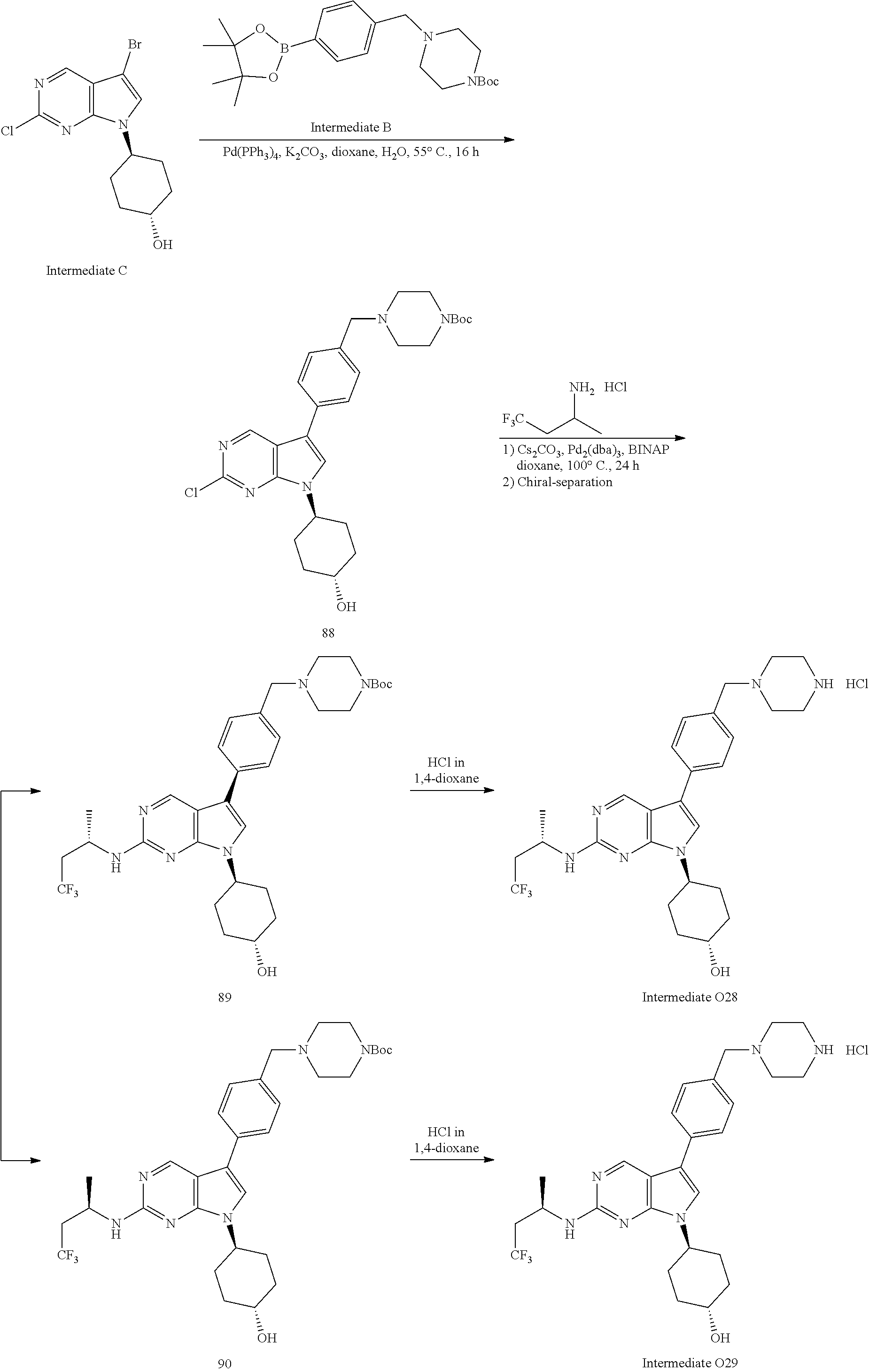

### Step 1: tert-butyl 4-[(4-[2-chloro-7-[trans-4-hydroxycyclohexyl]-7H-pyrrolo[2,3-d]pyrimidin-5-yl]phenyl)methyl]piperazine-1-carboxylate

To a stirred solution of trans-4-[5-bromo-2-chloro-7H-pyrrolo[2,3-d]pyrimidin-7-yl]cyclohexan-1-ol (1.00 g, 3.03 mmol) and tert-butyl 4-[[4-(4,4,5,5-tetramethyl-1,3,2-dioxaborolan-2-yl)phenyl]methyl]piperazine-1-carboxylate (2.07 g, 5.14 mmol) in 1,4-dioxane (30.0 mL) and H₂O (10.0 mL) were added K₂CO₃ (1.25 g, 9.07 mmol) and Pd(PPh₃)₄ (350 mg, 0.30 mmol) at room temperature. The resulting mixture was purged with nitrogen three times and stirred for 16 h at 55° C. under nitrogen atmosphere. The resulting mixture was diluted with water (20.0 mL) and extracted with EtOAc (3×50.0 mL). The combined organic layers were washed with brine (30.0 mL), dried over anhydrous Na₂SO₄. After filtration, the filtrate was concentrated under reduced pressure. The residue was purified by reversed phase flash chromatography with the following conditions: Column: WelFlash™ C18-I, 20-40 um, 330 g; Eluent A: water (plus 10 mmol/L NH₄HCO₃); Eluent B: ACN; Gradient: 50%-70% B in 25 min; Flow rate: 80 mL/min; Detector: UV 220/254 nm; desired fractions were collected at 69% B and concentrated under reduced pressure to afford tert-butyl 4-[(4-[2-chloro-7-[trans-4-hydroxycyclohexyl]-7H-pyrrolo[2,3-d]pyrimidin-5-yl]phenyl)methyl]piperazine-1-carboxylate (730 mg, 46%) as a white solid. ¹H NMR (400 MHz, DMSO-d₆) δ 9.25 (s, 1H), 8.24 (s, 1H), 7.79-7.71 (m, 2H), 7.43-7.34 (m, 2H), 4.75 (d, J=4.4 Hz, 1H), 4.65-4.53 (m, 1H), 3.63-3.46 (m, 3H), 2.51-2.45 (m, 6H), 2.39-2.26 (m, 4H), 2.07-1.87 (m, 6H), 1.39 (s, 9H). LC/MS (ESI, m/z): [(M+1)]⁺=526.30

### Step 2: tert-butyl 4-[(4-[7-[trans-4-hydroxycyclohexyl]-2-[[(2S)-4,4,4-trifluorobutan-2-yl]amino]-7H-pyrrolo[2,3-d]pyrimidin-5-yl]phenyl)methyl]piperazine-1-carboxylate and tert-butyl 4-[(4-[7-[trans-4-hydroxycyclohexyl]-2-[[(2R)-4,4,4-trifluorobutan-2-yl]amino]-7H-pyrrolo[2,3-d]pyrimidin-5-y]phenyl)methyl]piperazine-1-carboxylate

To a stirred solution of tert-butyl 4-[(4-[2-chloro-7-[trans-4-hydroxycyclohexyl]-7H-pyrrolo[2,3-d]pyrimidin-5-yl]phenyl)methyl]piperazine-1-carboxylate (400 mg, 0.76 mmol) and 4,4,4-trifluorobutan-2-amine hydrochloride (373 mg, 2.28 mmol) in 1,4-dioxane (15.0 mL) were added Cs₂CO₃ (743 mg, 2.28 mmol), Pd2(dba)3 (70 mg, 0.08 mmol) and BINAP (95 mg, 0.15 mmol). The reaction mixture was purged with N2 for three times and was stirred at 100° C. for 24 h under nitrogen atmosphere. The resulting mixture was cooled and concentrated under vacuum. The residue was purified by reversed phase flash chromatography with the following conditions: Column: WelFlash™ C18-I, 20-40 um, 330 g; Eluent A: water (plus 10 mmol/L TEA); Eluent B: ACN; Gradient: 50%-70% B in 20 min; Flow rate: 80 mL/min; Detector: UV 220/254 nm; desired fractions were collected at 63% B and concentrated under reduced pressure. The residue was carried out to chiral resolution to afford tert-butyl 4-[(4-[7-[trans-4-hydroxycyclohexyl]-2-[[(2S)-4,4,4-trifluorobutan-2-yl]amino]-7H-pyrrolo[2,3-d]pyrimidin-5-yl]phenyl)methyl]piperazine-1-carboxylate (81.6 mg, 17%) as a yellow solid, and tert-butyl 4-[(4-[7-[trans-4-hydroxycyclohexyl]-2-[[(2R)-4,4,4-trifluorobutan-2-yl]amino]-7H-pyrrolo[2,3-d]pyrimidin-5-yl]phenyl)methyl]piperazine-1-carboxylate (74.9 mg, 16%) as a yellow solid.

89: ¹H NMR (400 MHz, DMSO-d₆) δ 8.87 (s, 1H), 7.68-7.59 (m, 3H), 7.32 (d, J=8.0 Hz, 2H), 6.93 (d, J=8.0 Hz, 1H), 4.70 (s, 1H), 4.47-4.32 (m, 2H), 3.49 (s, 3H), 2.82-2.60 (m, 2H), 2.46-2.27 (m, 6H), 2.03-1.84 (m, 6H), 1.50-1.36 (m, 14H), 1.15-0.99 (m, 2H). LC/MS (ESI, m/z): [(M+1)]⁺=617.30

90: ¹H NMR (400 MHz, DMSO-d₆) δ 8.87 (s, 1H), 7.69-7.56 (m, 3H), 7.32 (d, J=7.9 Hz, 2H), 6.93 (d, J=8.1 Hz, 1H), 4.71 (s, 1H), 4.47-4.29 (m, 2H), 3.51 (s, 3H), 2.83-2.61 (m, 2H), 2.46-2.24 (m, 6H), 2.06-1.84 (m, 6H), 1.52-1.37 (m, 14H), 1.13-1.00 (m, 2H). LC/MS (ESI, m/z): [(M+1)]⁺=617.30

The following intermediates in Table 9 were synthesized according to the above procedure:

TABLE 9

| Inter- | | | MS: | |
|---|---|---|---|---|
| medi- | | | [(M + | |
| ate | Structure | Name | 1)]⁺ | ¹H NMR |
| 91 | 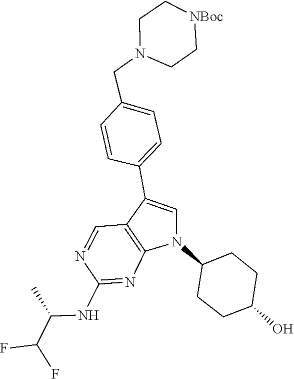 [SMILES: C[C@H](Nc1ncc2c(-c3ccc(CN4CCN(C(=O)OC(C)(C)C)CC4)cc3)cn([C@H]3CC[C@H](O)CC3)c2n1)C(F)F] | tert-butyl-4-[[4-(2- [[(2S)-1,1- difluoropropan-2- yl]amino]-7-[trans-4- hydroxycyclohexyl] pyrrolo[2,3-d]pyrimidin- 5- yl)phenyl]methyl] piperazine-1-carboxylate | 585.35 | (400 MHz, DMSO-d₆) δ 8.89 (s, 1H), 7.67-7.63 (m, 3H), 7.33 (d, J = 7.9 Hz, 2H), 7.07 (d, J = 8.0 Hz, 1H), 6.17-6.02 (m, 1H), 4.69 (d, J = 4.5 Hz, 1H), 4.43 (d, J = 13.0 Hz, 2H), 3.59-3.40 (m, 3H), 2.39-2.28 (m, 5H), 2.06-1.83 (m, 7H), 1.50-1.36 (m, 13H), 1.24 (d, J = 6.8 Hz, 3H). |
| 92 | 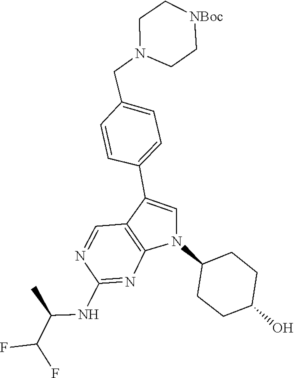 [SMILES: C[C@@H](Nc1ncc2c(-c3ccc(CN4CCN(C(=O)OC(C)(C)C)CC4)cc3)cn([C@H]3CC[C@H](O)CC3)c2n1)C(F)F] | tert-butyl-4-[[4-(2- [[(2R)-1,1- difluoropropan-2- yl]amino]-7-[trans-4- hydroxycyclohexyl] pyrrolo[2,3-d]pyrimidin- 5- yl)phenyl]methyl] piperazine-1-carboxylate | 585.35 | (400 MHz, DMSO-d₆) δ 8.89 (s, 1H), 7.65 (d, J = 8.5 Hz, 3H), 7.33 (d, J = 7.8 Hz, 2H), 7.07 (d, J = 8.0 Hz, 1H), 6.17- 6.00 (m, 1H), 4.69 (d, J = 4.5 Hz, 1H), 4.44 (s, 2H), 3.59-3.41 (m, 3H), 2.43-2.31 (m, 5H), 2.03-1.87 (m, 7H), 1.49-1.32 (m, 13H), 1.24 (d, J = 6.9 Hz, 3H). |
| 93 | 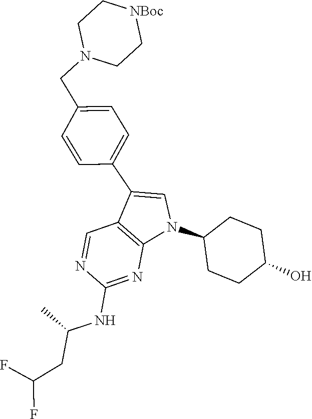 [SMILES: C[C@@H](CC(F)F)Nc1ncc2c(-c3ccc(CN4CCN(C(=O)OC(C)(C)C)CC4)cc3)cn([C@H]3CC[C@H](O)CC3)c2n1] | tert-butyl 4-[[4-(2- [[(2S)-4,4- difluorobutan-2- yl]amino]-7-[trans-4- hydroxycyclohexyl] pyrrolo[2,3-d]pyrimidin- 5- yl)phenyl]methyl] piperazine-1-carboxylate | 599.30 | (400 MHz, CD₃OD) δ 8.75 (s, 1H), 7.62 (d, J = 8.0 Hz, 2H), 7.45-7.37 (m, 3H), 6.05 (t, J = 57.4 Hz, 1H), 4.57 (s, 1H), 4.49-4.32 (m, 1H), 3.73 (d, J = 11.2 Hz, 1H), 3.58 (s, 2H), 3.49-4.31 (m, 4H), 2.57-2.42 (m, 4H), 2.23 (s, 1H), 2.19-2.00 (m, 7H), 1.55 (s, 2H), 1.47 (s, 9H), 1.36 (d, J = 6.6 Hz, 3H). |
| 94 | 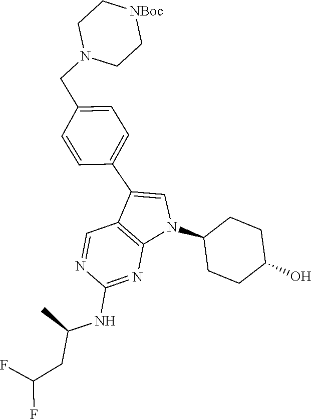 [SMILES: C[C@H](CC(F)F)Nc1ncc2c(-c3ccc(CN4CCN(C(=O)OC(C)(C)C)CC4)cc3)cn([C@H]3CC[C@H](O)CC3)c2n1] | tert-butyl 4-[[4-(2- [[(2R)-4,4- difluorobutan-2- yl]amino]-7-[trans-4- hydroxycyclohexyl] pyrrolo[2,3-d]pyrimidin- 5- yl)phenyl]methyl] piperazine-1-carboxylate | 599.30 | (400 MHz, CD₃OD) δ 8.74 (s, 1H), 7.62 (d, J = 8.0 Hz, 2H), 7.46-7.37 (m, 3H), 6.05 (s, 1H), 4.57 (s, 1H), 4.39 (d, J = 6.4 Hz, 1H), 3.74 (s, 1H), 3.58 (s, 2H), 3.56-3.41 (m, 4H), 2.56-2.41 (m, 4H), 2.23 (s, 1H), 2.19-2.00 (m, 7H), 1.57-1.42 (m, 11H), 1.36 (d, J = 6.7 Hz, 3H). |
| 95 | 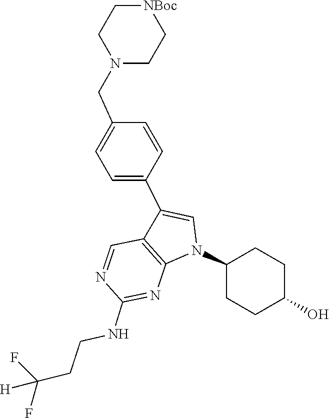 [SMILES: CC(C)(C)OC(=O)N1CCN(Cc2ccc(-c3cn([C@H]4CC[C@H](O)CC4)c4nc(NCCC(F)F)ncc34)cc2)CC1] | tert-butyl 4-[(4-[2- [(3,3- difluoropropyl)amino]- 7-[trans-4- hydroxycyclohexyl]- 7H-pyrrolo[2,3- d]pyrimidin-5- yl]phenyl)methyl] piperazine-1-carboxylate | 585.40 | (400 MHz, DMSO-d₆) δ 8.87 (s, 1H), 7.69-7.58 (m, 3H), 7.33 (d, J = 7.9 Hz, 2H), 7.01 (t, J = 5.9 Hz, 1H), 6.29-6.12 (m, 1H), 4.71 (s, 1H), 4.49-4.31 (m, 1H), 3.63-3.40 (m, 8H), 2.46-2.31 (m, 4H), 2.27-2.12 (m, 2H), 2.08 (s, 1H), 2.03-1.81 (m, 6H), 1.49-1.21 (m, 9H), 1.39-1.21 (m, 2H). |

### Step 3: tert-butyl 4-[(4-[7-[trans-4-hydroxycyclohexyl]-2-[[(2S)-4,4,4-trifluorobutan-2-yl]amino]-7H-pyrrolo[2,3-d]pyrimidin-5-yl]phenyl)methyl]piperazine-1-carboxylate hydrochloride

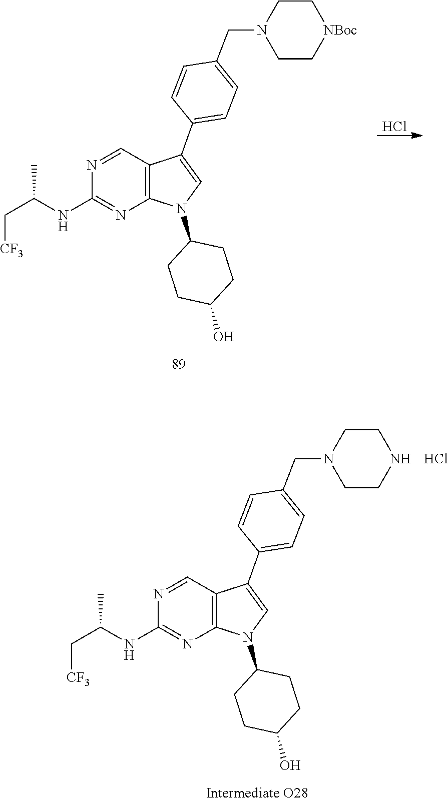 [SMILES: C[C@@H](CC(F)(F)F)Nc1ncc2c(-c3ccc(CN4CCN(C(=O)OC(C)(C)C)CC4)cc3)cn([C@H]3CC[C@H](O)CC3)c2n1 ; Cl ; C[C@@H](CC(F)(F)F)Nc1ncc2c(-c3ccc(CN4CCNCC4)cc3)cn([C@H]3CC[C@H](O)CC3)c2n1 ; Cl]

A solution of tert-butyl 4-[(4-[7-[trans-4-hydroxycyclohexyl]-2-[[(2S)-4,4,4-trifluorobutan-2-yl] amino]-7H-pyrrolo[2,3-d]pyrimidin-5-yl]phenyl)methyl]piperazine-1-carboxylate (81.6 mg, 0.13 mmol) in 1,4-dioxane (5.00 mL) and HCl (4 M in 1,4-dioxane, 5.00 mL) was stirred for 2 h at room temperature under nitrogen atmosphere. The resulting mixture was concentrated under vacuum. to afford trans-4-(5-[4-[(piperazin-1-yl)methyl]phenyl]-2-[[(2S)-4,4,4-trifluorobutan-2-yl]amino]-7H-pyrrolo[2,3-d]pyrimidin-7-yl)cyclohexan-1-ol hydrochloride (65 mg, 95%) as a yellow solid. ¹H NMR (400 MHz, CD₃OD) δ 8.88 (s, 1H), 8.01 (s, 1H), 7.81 (d, J=7.8 Hz, 2H), 7.72 (d, J=8.2 Hz, 2H), 4.71-4.60 (m, 2H), 4.40 (s, 1H), 3.62 (s, 7H), 2.82-2.64 (m, 1H), 2.63-2.49 (m, 2H), 2.16 (s, 2H), 2.21-2.02 (m, 4H), 1.56 (s, 2H), 1.50 (d, J=6.8 Hz, 3H), 1.36-1.27 (m, 2H). LC/MS (ESI, m/z): [(M+1)]⁺=517.30

The following intermediates in Table 10 were synthesized according to the above procedure:

TABLE 10

| Inter- | | | | |
|---|---|---|---|---|
| medi- | | | MS: | |
| ate | Structure | Name | [(M + 1)]⁺ | ¹H NMR |
| O29 | 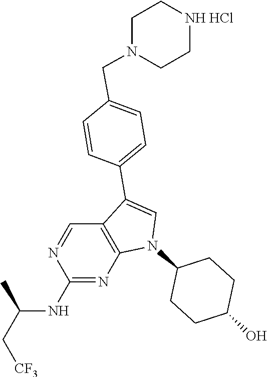 | trans-4-(5-[4-[(piperazin- 1-yl)methyl]phenyl]-2- [[2R)-4,4,4- trifluorobutan-2- yl]amino]-7H- pyrrolo[2,3-d]pyrimidin- 7-yl)cyclohexan-1-ol hydrochloride | 517.30 | (400 MHz, DMSO-d₆) δ 9.06 (s, 1H), 8.04 (s, 1H), 7.82 (d, J = 8.0 Hz, 2H), 7.69 (s, 2H), 4.49-4.35 (m, 3H), 3.02-2.83 (m, 1H), 2.74 (s, 1H), 2.09-1.83 (m, 6H), 1.46-1.31 (m, 4H), 1.26- 1.15 (m, 1H). |
| O30 | 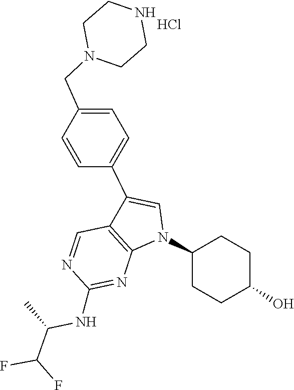 [SMILES: C[C@H](Nc1ncc2c(-c3ccc(CN4CCNCC4)cc3)cn([C@H]3CC[C@H](O)CC3)c2n1)C(F)F ; Cl] | trans-4-(2-[[(2S)-1,1- difluoropropan-2- yl]amino]-5-[4- (piperazin-1- ylmethyl)phenyl]pyrrolo [2,3-d]pyrimidin-7- yl)cyclohexan-1-ol hydrochloride | 485.25 | crude to next step directly without further purification |
| O31 | 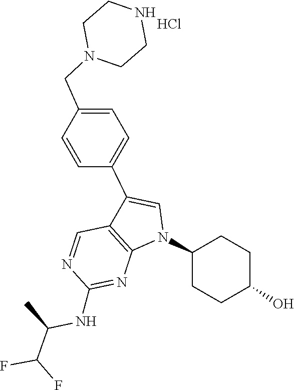 [SMILES: C[C@@H](Nc1ncc2c(-c3ccc(CN4CCNCC4)cc3)cn([C@H]3CC[C@H](O)CC3)c2n1)C(F)F ; Cl] | trans-4-(2-[[(2R)-1,1- difluoropropan-2- yl]amino]-5-[4- (piperazin-1- ylmethyl)phenyl]pyrrolo [2,3-d]pyrimidin-7- yl)cyclohexan-1-ol hydrochloride | 485.25 | crude to next step directly without further purification |
| O32 | 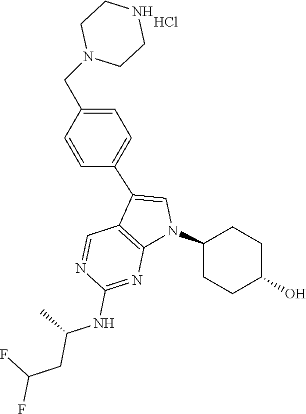 [SMILES: C[C@@H](CC(F)F)Nc1ncc2c(-c3ccc(CN4CCNCC4)cc3)cn([C@H]3CC[C@H](O)CC3)c2n1 ; Cl] | trans-4-(2-[[(2S)-1,1- difluorobutan-2- yl]amino]-5-[4- (piperazin-1- ylmethyl)phenyl]pyrrolo [2,3-d]pyrimidin-7- yl)cyclohexan-1-ol hydrochloride | 499.25 | crude to next step directly without further purification |
| O33 | 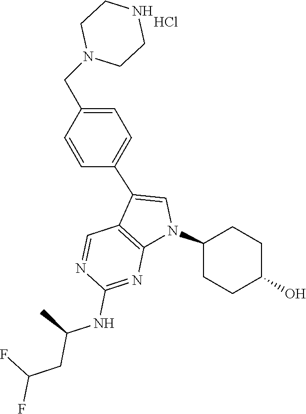 [SMILES: C[C@H](CC(F)F)Nc1ncc2c(-c3ccc(CN4CCNCC4)cc3)cn([C@H]3CC[C@H](O)CC3)c2n1 ; Cl] | trans-4-(2-[[(2R)-4,4- difluorobutan-2- yl]amino]-5-[4- (piperazin-1- ylmethyl)phenyl]pyrrolo [2,3-d]pyrimidin-7- yl)cyclohexan-1-ol hydrochloride | 499.25 | crude to next step directly without further purification |
| O34 | 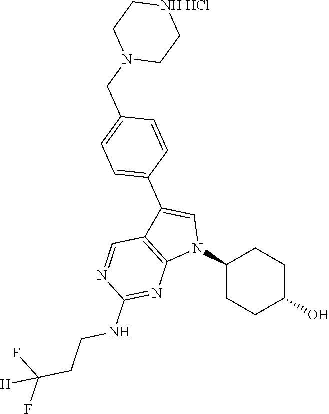 | trans-4-[2-[(3,3- difluoropropyl)amino]-5- [4-[(piperazin-1- yl)methyl]phenyl]-7H- pyrrolo[2,3-d]pyrimidin- 7-yl]cyclohexan-1-ol hydrochloride | 485.35 | (400 MHz, DMSO-d₆) δ 9.13 (s, 1H), 8.18 (m, 2H), 7.84 (d, J = 8.0 Hz, 2H), 7.76 (d, J = 8.0 Hz, 2H), 6.29-6.15 (m, 1H), 4.56- 4.39 (m, 4H), 3.70-3.36 (m, 12H), 2.31-2.15 (m, 2H), 2.08-1.86 (m, 6H), 1.49- 1.30 (m, 2H). |

### INTERMEDIATE 96: tert-butyl 3-(2-oxoethoxy)azetidine-1-carboxylate

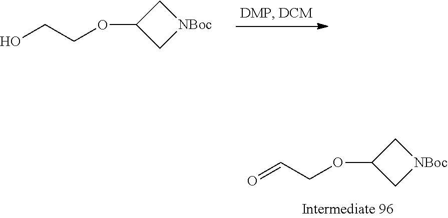 [SMILES: CC(C)(C)OC(=O)N1CC(OCCO)C1 ; CC(C)(C)OC(=O)N1CC(OCC=O)C1]

A solution of tert-butyl 3-(2-hydroxyethoxy)azetidine-1-carboxylate (2.00 g, 9.21 mmol) in DCM (20.0 mL) was treated with DMP (4.29 g, 10.1 mmol) for 1 h at room temperature under nitrogen atmosphere. Hexane (40 mL) was added to the resulting mixture and stirred for 10 min. The resulting mixture was filtered and the filtrate was concentrated under reduced pressure. The residue was purified by silica gel column chromatography, eluted with EtOAc in petroleum ether (50%-100%) to afford tert-butyl 3-(2-oxoethoxy)azetidine-1-carboxylate (1.50 g, 76%) as a yellow oil. ¹H NMR (400 MHz, DMSO-d₆) δ 9.55 (s, 1H), 4.31 (tt, J=6.4, 4.1 Hz, 1H), 4.21 (s, 2H), 4.11-3.96 (m, 4H), 1.46 (s, 9H).

The following intermediates in Table 11 were synthesized according to the above procedure:

TABLE 11

| Intermediate | Structure | Name | MS: [(M + 1)]⁺ |
|---|---|---|---|
| 97 | 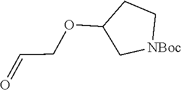 [SMILES: CC(C)(C)OC(=O)N1CCC(OCC=O)C1] | tert-butyl 3-(2- oxoethoxy)pyrrolidine-1- carboxylate | 229.40 |

### INTERMEDIATE O35: trans-4-[5-(4-[[4,4-bipiperidin]-1-ylmethyl]phenyl)-2-[(3,3,3-trifluoropropyl)amino]pyrrolo[2,3-d]pyrimidin-7-yl]cyclohexan-1-ol hydrochloride

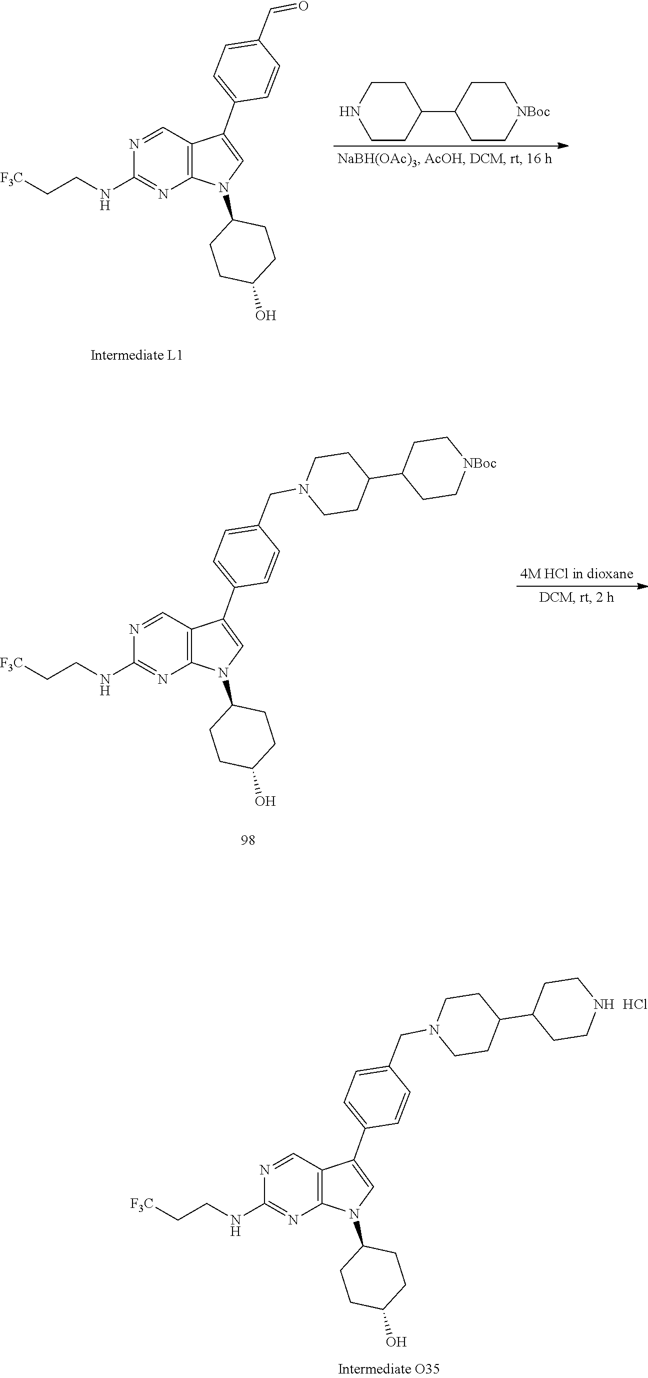 [SMILES: O=Cc1ccc(-c2cn([C@H]3CC[C@H](O)CC3)c3nc(NCCC(F)(F)F)ncc23)cc1 ; CC(C)(C)OC(=O)N1CCC(C2CCNCC2)CC1 ; CC(C)(C)OC(=O)N1CCC(C2CCN(Cc3ccc(-c4cn([C@H]5CC[C@H](O)CC5)c5nc(NCCC(F)(F)F)ncc45)cc3)CC2)CC1 ; O[C@H]1CC[C@H](n2cc(-c3ccc(CN4CCC(C5CCNCC5)CC4)cc3)c3cnc(NCCC(F)(F)F)nc32)CC1 ; Cl]

### Step 1: trans-tert-butyl 1-[(4-[7-H-hydroxycyclohexyl]-2-[(3,3,3-trifluoropropyl)amino]pyrrolo[2,3-d]pyrimidin-5-yl]phenyl)methyl]-[4,4-bipiperidine]-1-carboxylate

To a stirred mixture of trans-4-[7-[4-hydroxycyclohexyl]-2-[(3,3,3-trifluoropropyl)amino]pyrrolo[2,3-d]pyrimidin-5-yl]benzaldehyde (150 mg, 0.35 mmol), AcOH (42 mg, 0.69 mmol) and tert-butyl [4,4-bipiperidine]-1-carboxylate (140 mg, 0.52 mmol) in DCM (5.00 mL)/DMA (1.00 mL) was added NaBH(OAc)₃ (147 mg, 0.69 mmol). The resulting solution was stirred for 16 h at room temperature. Upon completion, the resulting solution was concentrated under reduced pressure. The residue was purified by reversed phase flash chromatography with the following conditions: Column: WelFlash™ C18-I, 20-40 um, 80 g; Eluent A: water (plus 10 mmol/L NH₄HCO₃); Eluent B: ACN; Gradient: 65%-95% B in 15 min; Flow rate: 50 mL/min; Detector: UV 220/254 nm; desired fractions were collected at 95% B and concentrated under reduced pressure to afford trans-tert-butyl 1-[(4-[7-[4-hydroxycyclohexyl]-2-[(3,3,3-trifluoropropyl)amino]pyrrolo[2,3-d]pyrimidin-5-yl]phenyl)methyl]-[4,4-bipiperidine]-1-carboxylate (80 mg, 34%) as a light yellow solid. ¹H NMR (400 MHz, DMSO-d₆) δ 8.88 (s, 1H), 7.67-7.59 (m, 3H), 7.30 (d, J=7.8 Hz, 2H), 7.05 (t, J=5.9 Hz, 1H), 4.70 (d, J=4.5 Hz, 1H), 4.46-4.38 (m, 1H), 3.95 (d, J=11.1 Hz, 2H), 3.57 (q, J=6.7 Hz, 3H), 3.42 (s, 2H), 2.85 (d, J=10.9 Hz, 2H), 2.66 (s, 1H), 2.70-2.54 (m, 5H), 2.08-1.92 (m, 4H), 1.99-1.82 (m, 4H), 1.73-1.62(m, 4H), 1.39 (s, 9H), 1.32-1.12 (m, 4H), 1.04-0.97 (m, 2H). LC/MS (ESI, m/z): [(M+1)]⁺=685.60.

The following intermediates in Table 12 were synthesized according to the above procedure:

TABLE 12

| Inter- | | | MS: | |
|---|---|---|---|---|
| medi- | | | [(M + | |
| ate | Structure | Name | 1)]⁺ | ¹H NMR |
| 98-1 | 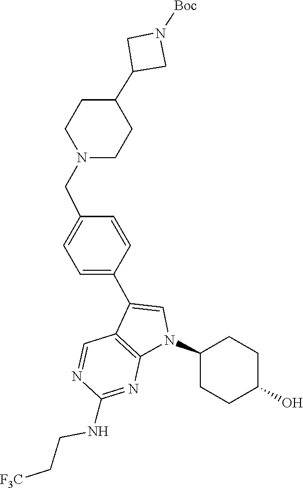 [SMILES: CC(C)(C)OC(=O)N1CC(C2CCN(Cc3ccc(-c4cn([C@H]5CC[C@H](O)CC5)c5nc(NCCC(F)(F)F)ncc45)cc3)CC2)C1] | tert-butyl 3-[1-[(4-[7- [trans-4- hydroxycyclohexyl]- 2-[(3,3,3- trifluoropropyl)amino] pyrrolo[2,3- d]pyrimidin-5- yl]phenyl)methyl] piperidin-4-yl]azetidine- 1-carboxylate | 657.35 | (400 MHz, DMSO-d₆) δ 8.89 (s, 1H), 8.15 (s, 1H), 7.66 (d, J = 8.2 Hz, 3H), 7.35 (d, J = 7.9 Hz, 2H), 7.06 (t, J = 5.9 Hz, 1H), 4.70 (s, 1H), 4.48-4.38 (m, 1H), 3.84 (s, 2H), 3.64-3.49 (m, 7H), 3.22-3.13 (m, 1H), 2.92 (d, J = 11.0 Hz, 2H), 2.66-2.55 (m, 2H), 2.25 (d, J = 7.6 Hz, 1H), 2.08 (s, 2H), 2.05-1.82 (m, 7H), 1.67-1.51 (m, 3H), 1.37 (s, 13H), 1.19-1.00 (m, 2H), 0.94 (t, J = 7.3 Hz, 1H) |
| 98-2 | 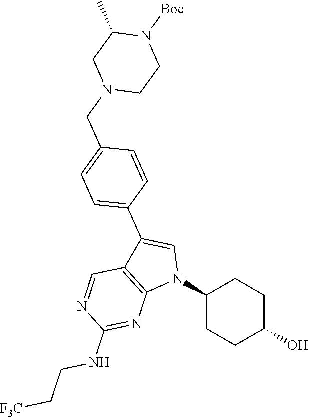 [SMILES: C[C@H]1CN(Cc2ccc(-c3cn([C@H]4CC[C@H](O)CC4)c4nc(NCCC(F)(F)F)ncc34)cc2)CCN1C(=O)OC(C)(C)C] | tert-butyl (2S)-2- methyl-4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazine-1-carboxylate | 617.30 | (400 MHz, CD₃OD) δ 8.76 (s, 1H), 7.61 (d, J = 8.0 Hz, 2H), 7.46- 7.38 (m, 3H), 4.65-4.53 (m, 1H), 4.20 (s, 1H), 3.82 (d, J = 13.1 Hz, 1H), 3.84-3.65 (m, 3H), 3.59 (d, J = 13.1 Hz, 1H), 3.46 (d, J = 13.0 Hz, 1H), 3.15 (t, J = 11.8 Hz, 1H), 2.86 (d, J = 11.5 Hz, 1H), 2.72 (d, J = 11.4 Hz, 1H), 2.69-2.55 (m, 2H), 2.17-2.01 (m, 8H), 1.69-1.56 (m, 2H), 1.48 (s, 9H), 1.28 (d, J = 6.7 Hz, 3H) |
| 98-3 | 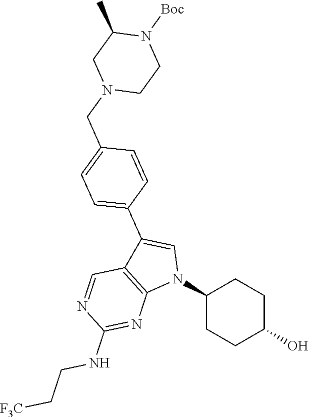 [SMILES: C[C@@H]1CN(Cc2ccc(-c3cn([C@H]4CC[C@H](O)CC4)c4nc(NCCC(F)(F)F)ncc34)cc2)CCN1C(=O)OC(C)(C)C] | tert-butyl (2R)-2- methyl-4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazine-1-carboxylate | 617.30 | (400 MHz, CD₃OD) δ 8.76 (s, 1H), 7.61 (d, J = 8.1 Hz, 2H), 7.46- 7.38 (m, 3H), 4.68-4.55 (m, 1H), 4.20 (s, 1H), 3.82 (d, J = 13.2 Hz, 1H), 3.84-3.65 (m, 3H), 3.59 (d, J = 13.1 Hz, 1H), 3.46 (d, J = 13.1 Hz, 1H), 3.15 (t, J = 11.8 Hz, 1H), 2.86 (d, J = 11.6 Hz, 1H), 2.72 (d, J = 11.3 Hz, 1H), 2.68-2.55 (m, 2H), 2.19-2.02 (m, 8H), 1.59-1.48 (m, 2H), 1.47 (s, 9H), 1.27 (d, J = 6.8 Hz, 3H) |
| 98-4 | 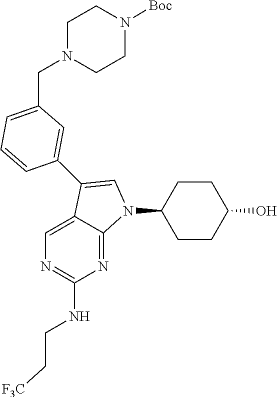 [SMILES: CC(C)(C)OC(=O)N1CCN(Cc2cccc(-c3cn([C@H]4CC[C@H](O)CC4)c4nc(NCCC(F)(F)F)ncc34)c2)CC1] | tert-butyl 4-[(3-[7- [trans-4- hydroxycyclohexyl]- 2-[(3,3,3- trifluoropropyl)amino] pyrrolo[2,3- d]pyrimidin-5- yl]phenyl)methyl] piperazine-1-carboxylate | 603.35 | (400 MHz, CD₃OD) δ 8.78 (s, 1H), 7.64 (d, J = 1.8 Hz, 1H), 7.56 (d, J = 7.9 Hz, 1H), 7.45 (s, 1H), 7.39 (t, J = 7.6 Hz, 1H), 7.24 (d, J = 7.6 Hz, 1H), 4.67-4.46 (m, 1H), 3.77-3.67 (m, 3H), 3.61 (s, 2H), 3.56-3.45 (m, 4H), 2.68-2.52 (m, 2H), 2.58-2.49 (m, 4H), 2.16-2.04 (m, 6H), 1.59-1.45 (m, 2H), 1.47 (s, 9H) |
| 98-5 | 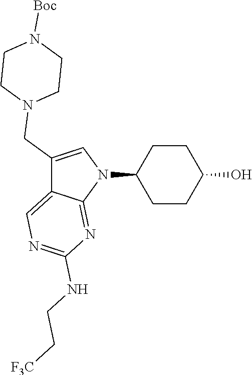 [SMILES: CC(C)(C)OC(=O)N1CCN(Cc2cn([C@H]3CC[C@H](O)CC3)c3nc(NCCC(F)(F)F)ncc23)CC1] | tert-butyl-4-([7- [trans-4- hydroxycyclohexyl]- 2-[(3,3,3- trifluoropropyl)amino] pyrrolo[2,3- d]pyrimidin-5- yl]methyl)piperazine- 1-carboxylate. | 527.25 | (400 MHz, CD₃OD) δ 8.60 (s, 1H), 7.07 (s, 1H), 4.47 (m, 1H), 3.76-3.66 (m, 3H), 3.64 (s, 2H), 3.54-3.35 (m, 4H), 2.59-2.45 (m, 2H), 2.55-2.38 (m, 4H), 2.12 (d, J = 12.5 Hz, 2H), 2.07-1.92 (m, 5H), 1.59-1.46 (m, 1H), 1.46 (s, 9H) |
| 98-6 | 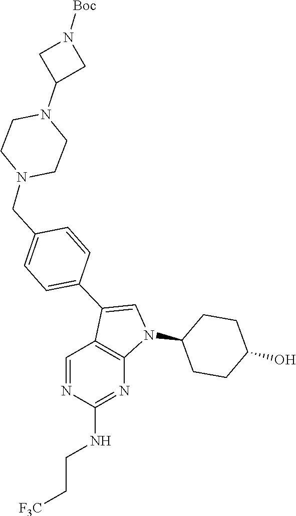 [SMILES: CC(C)(C)OC(=O)N1CC(N2CCN(Cc3ccc(-c4cn([C@H]5CC[C@H](O)CC5)c5nc(NCCC(F)(F)F)ncc45)cc3)CC2)C1] | tert-butyl-3-[4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazin-1-yl]azetidine- 1-carboxylate | 658.30 | (400 MHz, CD₃OD) δ 8.76 (s, 1H), 7.62 (d, J = 8.1 Hz, 2H), 7.45 (s, 1H), 7.40 (d, J = 8.1 Hz, 2H), 4.59-4.45 (m, 1H), 3.97 (s, 2H), 3.79 (d, J = 7.5 Hz, 2H), 3.76-3.68 (m, 3H), 3.59 (s, 2H), 3.19-3.08 (m, 1H), 2.71-2.30 (m, 9H), 2.20- 1.97 (m, 7H), 1.62-1.50 (m, 2H), 1.45 (s, 9H) |
| 98-7 | 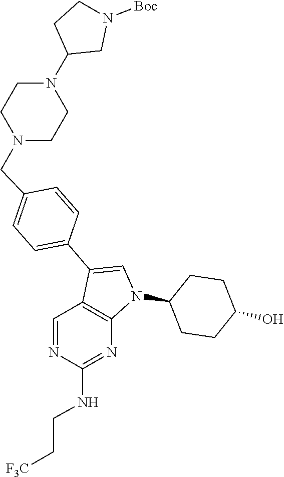 [SMILES: CC(C)(C)OC(=O)N1CCC(N2CCN(Cc3ccc(-c4cn([C@H]5CC[C@H](O)CC5)c5nc(NCCC(F)(F)F)ncc45)cc3)CC2)C1] | tert-butyl-3-[4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]pyrrolidine-1- carboxylate. | 672.40 | (400 MHz, CD₃OD) δ 8.76 (s, 1H), 7.62 (d, J = 8.0 Hz, 2H), 7.47- 7.36 (m, 3H), 4.61-4.52 (m, 1H), 3.80-3.68 (m, 3H), 3.63 (t, J = 7.7 Hz, 2H), 3.58 (s, 2H), 3.55 (s, 1H), 3.52 (d, J = 9.8 Hz, 1H), 3.38 (d, J = 9.6 Hz, 1H), 3.27 (s, 1H), 3.11- 3.03 (m, 1H), 2.86 (s, 1H), 2.69- 2.55 (m, 1H), 2.18-2.01 (m, 5H), 2.05 (s, 2H), 1.79-1.65 (m, 1H), 1.57 (s, 1H), 1.59-1.48 (m, 4H), 1.48-1.42 (m, 12H), 1.31 (s, 1H) |
| 98-8 | 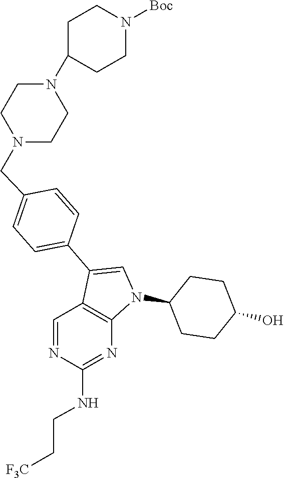 [SMILES: CC(C)(C)OC(=O)N1CCC(N2CCN(Cc3ccc(-c4cn([C@H]5CC[C@H](O)CC5)c5nc(NCCC(F)(F)F)ncc45)cc3)CC2)CC1] | tert-butyl-4-[4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]piperidine-1- carboxylate | 686.35 | (400 MHz, CD₃OD) δ 8.76 (s, 1H), 7.65-7.59 (m, 2H), 7.45 (s, 1H), 7.40 (d, J = 8.2 Hz, 2H), 4.56 (p, J = 8.5 Hz, 1H), 4.13 (d, J = 13.3 Hz, 2H), 3.79-3.65 (m, 3H), 3.58 (s, 2H), 2.87-2.47 (m, 12H), 2.46-2.37 (m, 1H), 2.15 (d, J = 10.8 Hz, 2H), 2.12-2.03 (m, 4H), 1.91 (d, J = 12.3 Hz, 2H), 1.61- 1.50 (m, 2H), 1.47 (s, 9H), 1.40- 1.31 (m, 2H) |
| 98-9 | 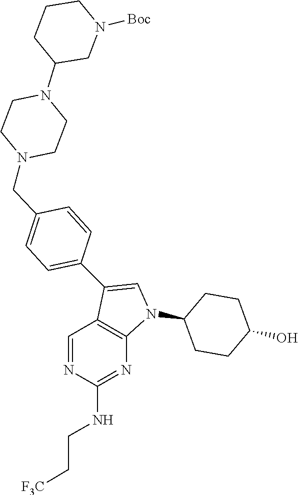 [SMILES: CC(C)(C)OC(=O)N1CCCC(N2CCN(Cc3ccc(-c4cn([C@H]5CC[C@H](O)CC5)c5nc(NCCC(F)(F)F)ncc45)cc3)CC2)C1] | tert-butyl-3-[4-[(4-[7- [trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]piperidine-1- carboxylate | 686.40 | (400 MHz, CD₃OD) δ 8.76 (s, 1H), 7.62 (d, J = 8.1 Hz, 2H), 7.47- 7.37 (m, 3H), 4.69-4.52 (m, 5H), 4.17 (s, 1H), 3.91 (d, J = 13.1 Hz, 1H), 3.77-3.69 (m, 3H), 3.58 (s, 2H), 3.47-3.34 (m, 6H), 2.86-2.65 (m, 3H), 2.79-2.52 (m, 7H), 2.64- 2.50 (m, 6H), 2.27 (s, 2H), 2.12- 2.05 (m, 5H), 1.77 (s, 1H), 1.58 (d, J = 11.4 Hz, 1H), 1.57-1.40 (m, 10H) |
| 98- 10 | 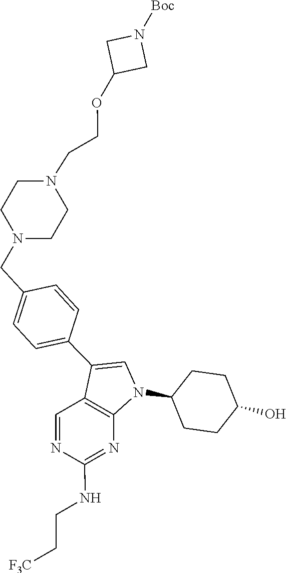 [SMILES: CC(C)(C)OC(=O)N1CC(OCCN2CCN(Cc3ccc(-c4cn([C@H]5CC[C@H](O)CC5)c5nc(NCCC(F)(F)F)ncc45)cc3)CC2)C1] | tert-butyl-3-(2-[4-[(4- [7-[trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]ethoxy)azetidine- 1-carboxylate | 702.50 | (400 MHz, DMSO-d₆) δ 8.88 (s, 1H), 7.67-7.60 (m, 3H), 7.31 (d, J = 7.9 Hz, 2H), 7.06 (d, J = 6.1 Hz, 1H), 4.71 (d, J = 4.5 Hz, 1H), 4.42 (s, 1H), 4.22 (s, 2H), 4.26-4.14 (m, 1H), 3.99-3.92 (m, 3H), 3.69-3.60 (m, 3H), 3.59-3.50 (m, 4H), 3.47- 3.40 (m, 4H), 2.69-2.50 (m, 2H), 2.45 (t, J = 5.9 Hz, 2H), 2.07-1.90 (m, 4H), 1.99-1.85 (m, 4H), 1.47- 1.31 (m, 12H) |
| 98- 11 | 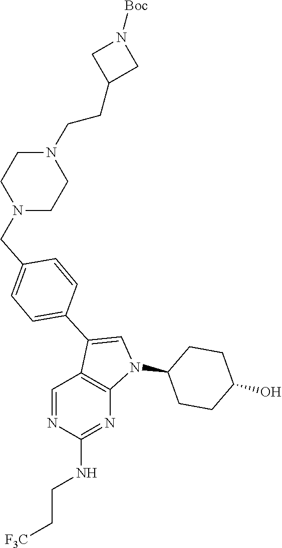 [SMILES: CC(C)(C)OC(=O)N1CC(CCN2CCN(Cc3ccc(-c4cn([C@H]5CC[C@H](O)CC5)c5nc(NCCC(F)(F)F)ncc45)cc3)CC2)C1] | tert-butyl-3-(2-[4-[(4- [7-[trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]ethyl)azetidine-1- carboxylate | 686.40 | (400 MHz, DMSO-d₆) δ 8.88 (s, 1H), 8.15 (s, 1H), 7.65 (d, J = 8.3 Hz, 2H), 7.33 (d, J = 7.9 Hz, 2H), 7.07 (t, J = 5.8 Hz, 1H), 4.47-4.37 (m, 1H), 3.89 (s, 2H), 3.67-3.45 (m, 6H), 3.56-3.31 (m, 3H), 2.74- 2.53 (m, 7H), 2.41-3.36 (m, 3H), 2.08 (s, 2H), 2.04-1.85 (m, 6H), 1.71 (q, J = 7.5 Hz, 2H), 1.47-1.31 (m, 11H) |
| 98- 12 | 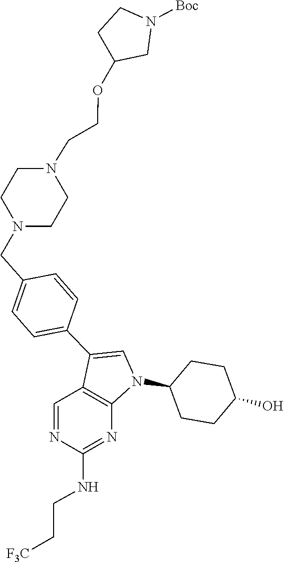 [SMILES: CC(C)(C)OC(=O)N1CCC(OCCN2CCN(Cc3ccc(-c4cn([C@H]5CC[C@H](O)CC5)c5nc(NCCC(F)(F)F)ncc45)cc3)CC2)C1] | tert-butyl-3-(2-[4-[(4- [7-[trans-4- hydroxycyclohexyl]- 2-[(3,3,3- trifluoropropyl)amino]- 7H-pyrrolo[2,3- d]pyrimidin-5- yl]phenyl)methyl] piperazin-1- yl]ethoxy)pyrrolidine- 1-carboxylate | 716.35 | (400 MHz, DMSO-d₆) δ 8.88 (s, 1H), 7.96 (s, 1H), 7.66-7.60 (m, 2H), 7.31 (d, J = 7.9 Hz, 2H), 7.06 (t, J = 5.9 Hz, 1H), 4.71 (d, J = 4.4 Hz, 2H), 4.01 (s, 1H), 3.45 (s, 2H), 2.99-2.91 (m, 4H), 2.74 (s, 2H), 2.69-2.51 (m, 4H), 2.49-2.35 (m, 5H), 2.49-2.31 (m, 4H), 2.08-1.99 (m, 4H), 1.99-1.82 (m, 4H), 1.49- 1.25 (m, 10H), 1.35 (s, 2H). |

### Step 2: trans-4-[5-(4-[[4,4-bipiperidin]-1-ylmethyl]phenyl)-2-[(3,3,3-trifluoropropyl)amino]pyrrolo[2,3-d]pyrimidin-7-yl]cyclohexan-1-ol hydrochloride

To a stirred solution of tert-butyl 1-[(4-[7-[trans-4-hydroxycyclohexyl]-2-[(3,3,3-trifluoropropyl)amino]pyrrolo[2,3-d]pyrimidin-5-yl]phenyl)methyl]-[4,4-bipiperidine]-1-carboxylate (80.0 mg, 0.12 mmol) in DCM (5.00 mL) was added HCl (4 M, in 1,4-dioxane, 5.00 mL) at room temperature under nitrogen atmosphere. The resulting solution was stirred for 2 h at room temperature. Upon completion, the reaction was concentrated under reduced pressure to give trans-4-[5-(4-[[4,4-bipiperidin]-1-ylmethyl]phenyl)-2-[(3,3,3-trifluoropropyl)amino]pyrrolo[2,3-d]pyrimidin-7-yl]cyclohexan-1-ol hydrochloride (80.0 mg, crude) as a light yellow solid. ¹H NMR (400 MHz, DMSO-d₆) δ 9.07 (s, 1H), 8.80 (d, J=10.9 Hz, 1H), 8.63 (s, 1H), 8.04 (s, 1H), 7.85-7.79 (m, 2H), 7.68 (t, J=5.4 Hz, 2H), 4.54-4.43 (m, 1H), 4.26 (d, J=5.2 Hz, 2H), 3.69 (d, J=9.4 Hz, 2H), 3.25 (d, J=12.2 Hz, 3H), 2.89-2.75 (m, 4H), 2.75-2.60 (m, 2H), 2.07-1.88 (m, 6H), 1.87-1.73 (m, 4H), 1.66-1.53 (m, 2H), 1.45-1.26 (m, 6H). LC/MS (ESI, m/z): [(M+1)]⁺=585.35.

The following intermediates in Table 13 were synthesized according to the above procedure:

TABLE 13

| Inter- | | | MS: | |
|---|---|---|---|---|
| medi- | | | [(M + | |
| ate | Structure | Name | 1)]⁺ | ¹H NMR |
| O36 | 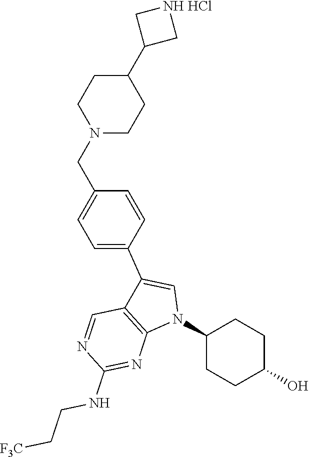 | trans-4-[5-(4-[[4- (azetidin-3- yl)piperidin-1- yl]methyl]phenyl)- 2-[(3,3,3- trifluoropropyl) amino]pyrrolo[2,3- d]pyrimidin-7- yl]cyclohexan-1-ol hydrochloride | 557.35 | (400 MHz, DMSO-d₆) δ 10.89 (s, 1H), 9.08 (s, 2H), 8.05 (s, 1H), 7.81 (d, J = 8.1 Hz, 2H), 7.69 (d, J = 8.0 Hz, 2H), 4.52-4.43 (m, 1H), 4.29 (d, J = 5.1 Hz, 2H), 3.94-3.88 (m, 2H), 3.79-2.65 (m, 5H), 3.56 (d, J = 11.1 Hz, 2H), 3.41-3.29 (m, 2H), 3.21-3.01 (m, 1H), 2.89-2.75 (m, 2H), 2.73-2.58 (m, 2H), 2.07- 1.89 (m, 6H), 1.88-1.72 (m, 2H), 1.52-1.30 (m, 4H) |
| O37 | 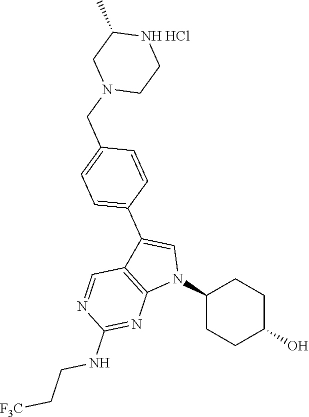 | trans-4-[5-(4- [[(3S)-3- Methylpiperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 517.30 | (400 MHz, CD₃OD) δ 8.95 (s, 1H), 8.04 (s, 1H), 7.88-7.76 (m, 4H), 4.65 (p, J = 8.2 Hz, 1H), 4.58 (s, 2H), 3.88 (t, J = 7.0 Hz, 2H), 3.80-3.72 (m, 4H), 3.78-3.56 (m, 5H), 3.62-3.53 (m, 1H), 3.40 (t, J = 12.1 Hz, 1H), 2.78-2.60 (m, 2H), 2.13-2.10 (m, 3H), 1.59-1.52 (m, 2H), 1.46 (d, J = 6.5 Hz, 3H) |
| O38 | 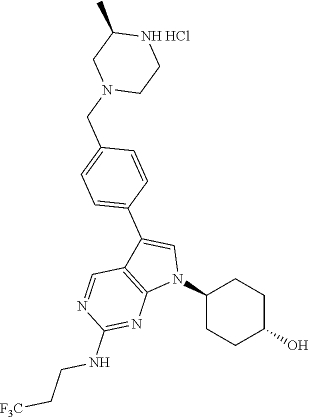 | trans-4-[5-(4- [[(3R)-3- Methylpiperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 517.30 | (400 MHz, CD₃OD) δ 8.95 (d, J = 3.2 Hz, 1H), 8.04 (s, 1H), 7.88- 7.76 (m, 4H), 4.70-4.61 (m, 1H), 4.58 (d, J = 4.1 Hz, 2H), 3.93 (s, 1H), 3.88 (t, J = 6.9 Hz, 2H), 3.79- 3.73 (m, 4H), 3.66 (s, 1H), 3.64- 3.51 (m, 2H), 3.39 (d, J = 12.9 Hz, 1H), 2.78-2.55 (m, 2H), 2.19 (s, 1H), 2.16-1.98 (m, 6H), 1.59-1.49 (m, 2H), 1.45 (d, J = 6.4 Hz, 3H) |
| O39 | 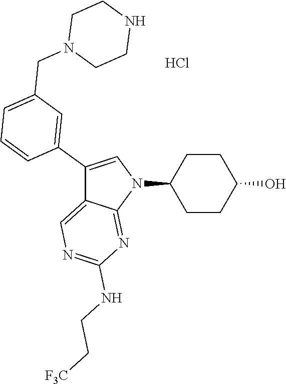 [SMILES: O[C@H]1CC[C@H](n2cc(-c3cccc(CN4CCNCC4)c3)c3cnc(NCCC(F)(F)F)nc32)CC1 ; Cl] | trans-4-[5-[3- (piperazin-1- ylmethyl)phenyl]- 2-[(3,3,3- trifluoropropyl) amino]pyrrolo[2,3- d]pyrimidin-7- yl]cyclohexan-1-ol hydrochloride. | 503.35 | (400 MHz, CD₃OD) δ 8.19-8.02 (m, 3H), 7.85 (d, J = 7.4 Hz, 1H), 7.68-7.55 (m, 2H), 4.75 (d, J = 3.3 Hz, 2H), 4.29-4.10 (m, 3H), 3.79- 3.65 (m, 4H), 3.51 (q, J = 7.0 Hz, 2H), 2.78-2.65 (m, 3H), 2.19-2.00 (m, 6H), 1.59-1.48 (m, 2H), 1.20 (t, J = 7.0 Hz, 2H) |
| O40 | 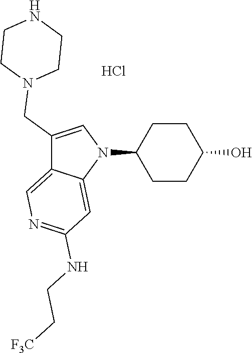 [SMILES: O[C@H]1CC[C@H](n2cc(CN3CCNCC3)c3cnc(NCCC(F)(F)F)cc32)CC1 ; Cl] | trans-4-[5- (piperazin-1- ylmethyl)-2-[(3,3,3- trifluoropropyl) amino]pyrrolo[2,3- d]pyrimidin-7- yl]cyclohexan-1-ol hydrochloride | 427.25 | (400 MHz, CD₃OD) δ 8.11 (s, 1H), 8.00 (s, 1H), 4.75 (d, J = 3.4 Hz, 2H), 4.67 (s, 2H), 3.51 (q, J = 7.0 Hz, 3H), 2.69-2.55 (m, 3H), 2.19-1.99 (m, 8H), 1.59-1.46 (m, 3H), 1.20 (t, J = 7.0 Hz, 3H) |
| O41 | 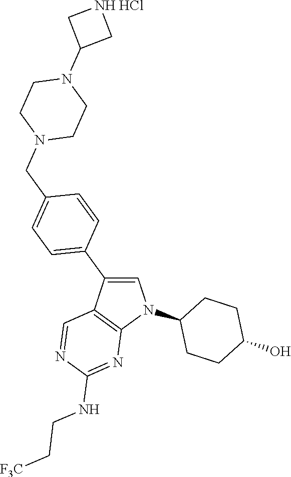 | trans-4-[5-(4-[[4- (Azetidin-3- yl)piperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 558.40 | (400 MHz, CD₃OD) δ 8.94 (s, 1H), 8.03 (s, 1H), 7.84 (d, J = 8.0 Hz, 2H), 7.72 (d, J = 8.0 Hz, 2H), 4.69-4.59 (m, 1H), 4.46 (s, 2H), 4.19-4.08 (m, 2H), 4.08-3.95 (m, 2H), 3.88 (t, J = 7.0 Hz, 2H), 3.80- 3.70 (m, 1H), 3.64-3.53 (m, 2H), 3.53 (s, 1H), 3.07 (s, 1H), 2.78- 2.52 (m, 2H), 2.50 (s, 1H), 2.19 (s, 1H), 2.12 (s, 3H), 1.59-1.45 (m, 2H) |
| O42 | 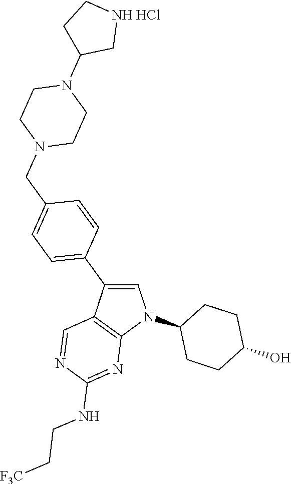 | trans-4-[5-(4-[[4- (Pyrrolidin-3- yl)piperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 572.30 | (400 MHz, CD₃OD) δ 8.95 (s, 1H), 8.04 (s, 1H), 7.87-7.81 (m, 2H), 7.74 (d, J = 8.0 Hz, 2H), 4.64 (q, J = 7.9 Hz, 1H), 4.49 (s, 2H), 3.88 (t, J = 7.0 Hz, 2H), 3.83-3.64 (m, 3H), 3.64-3.50 (m, 7H), 3.48- 3.35 (m, 5H), 2.78-2.56 (m, 2H), 2.35 (s, 1H), 2.19 (s, 2H), 2.17- 2.03 (m, 6H), 1.59-1.45 (m, 3H) |
| O43 | 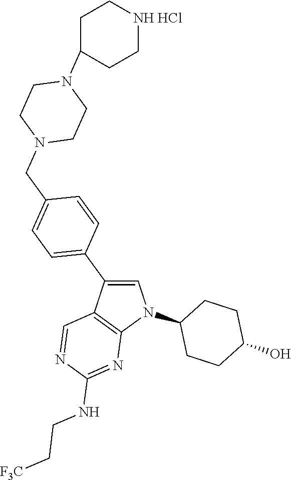 | trans-4-[5-(4-[[4- (piperidin-4- yl)piperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 586.30 | (400 MHz, CD₃OD) δ 8.97 (s, 1H), 8.04 (s, 1H), 7.86-7.76 (m, 4H), 4.90-4.65 (m, 2H), 4.56 (s, 1H), 3.90-3.59 (m, 15H), 3.17- 3.10 (m, 2H), 2.72-2.64 (m, 2H), 2.48-2.45 (m, 2H), 2.19-2.06 (m, 8H), 1.58-1.54 (m, 2H) |
| O44 | 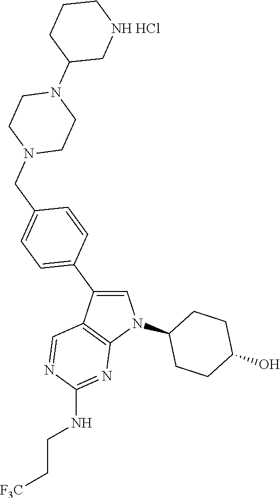 | trans-4-[5-(4-[[4- (piperidin-4- yl)piperazin-1- yl]methyl]phenyl)- 2-[(3,3,3- trifluoropropyl)amino]- 7H-pyrrolo[2,3- d]pyrimidin-7- yl]cyclohexan-1-ol hydrochloride | 586.30 | (400 MHz, CD₃OD) δ 8.93 (s, 1H), 8.03 (s, 1H), 7.85-7.83 (m, 2H), 7.72-7.70 (m, 2H), 4.88-4.63 (m, 2H), 4.52 (s, 1H), 3.92-3.58 (m, 15H), 3.16-3.08 (m, 2H), 2.70- 2.62 (m, 2H), 2.47-2.42 (m, 2H), 2.18-2.02 (m, 8H), 1.57-1.52 (m, 2H) |
| O45 | 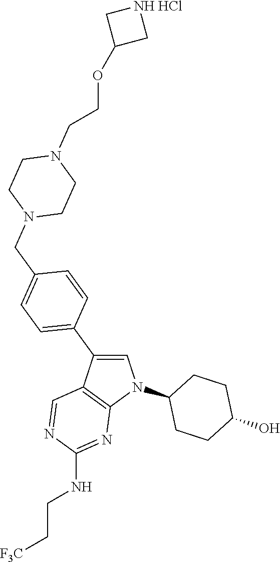 | trans-4-[5-[4-([4- [2-(azetidin-3- yloxy)ethyl] piperazin-1- yl]methyl)phenyl]- 2-[(3,3,3- trifluoropropyl)amino]- 7H-pyrrolo[2,3- d]pyrimidin-7- yl]cyclohexan-1-ol hydrochloride | 602.35 | (400 MHz, CD₃OD) δ 8.95 (d, J = 5.9 Hz, 1H), 8.08-7.98 (m, 1H), 7.88-7.75 (m, 4H), 4.59-4.50 (m, 3H), 4.39-4.22 (m, 2H), 4.23-4.11 (m, 2H), 3.99-3.84 (m, 6H), 3.88- 3.75 (m, 4H), 3.63-3.57 (m, 2H), 3.02 (s, 1H), 2.88 (s, 1H), 2.74- 2.63 (m, 1H), 2.35 (d, J = 11.9 Hz, 1H), 2.23 (d, J = 3.2Hz, 1H), 2.16- 2.09 (m, 1H), 1.89-1.80 (m, 1H), 1.59-1.45 (m, 1H) |
| O46 | 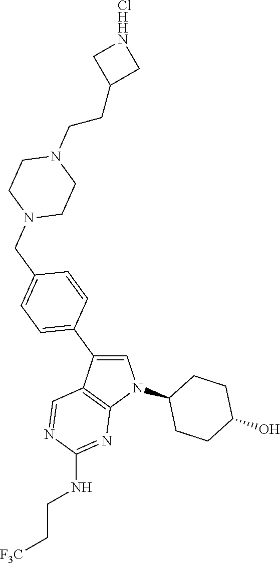 | trans-4-[5-[4-([4- [2-(azetidin-3- yl)ethyl]piperazin- 1- yl]methyl)phenyl]- 2-[(3,3,3- trifluoropropyl)amino]- 7H-pyrrolo[2,3- d]pyrimidin-7- yl]cyclohexan-1-ol hydrochloride | 586.40 | (400 MHz, DMSO-d₆) δ 9.14 (s, 1H), 8.24 (s, 1H), 8.15 (d, J = 6.2 Hz, 1H), 7.83 (d, J = 8.0 Hz, 2H), 7.78-7.66 (m, 2H), 4.55-4.27 (m, 4H), 3.96 (s, 2H), 3.85 (d, J = 10.1 Hz, 1H), 3.78-3.65 (m, 6H), 3.61- 3.42 (m, 7H), 3.08-3.01 (m, 1H), 2.86 (s, 1H), 2.77-2.55 (m, 2H), 2.09-1.90 (m, 8H), 1.47-1.32 (m, 2H), 1.29-1.15 (m, 1H) |
| O47 | 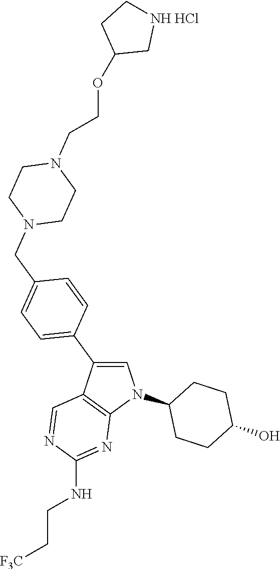 | trans-4-[5-[4-([4- [2-(pyrrolidin-3- yloxy)ethyl] piperazin-1- yl]methyl)phenyl]- 2-[(3,3,3- trifluoropropyl)amino]- 7H-pyrrolo[2,3- d]pyrimidin-7- yl]cyclohexan-1-ol | 616.30 | (400 MHz, CD₃OD) δ 8.95 (s, 1H), 8.02 (s, 1H), 7.85-7.82 (m, 2H), 7.77-7.75 (m, 2H), 4.89-4.70 (m, 1H), 4.65 (s, 2H), 4.49-4.35 (m, 1H), 3.93-3.86 (m, 12H), 3.76- 3.73 (m, 5H), 3.54-3.24 (m, 2H), 2.98-2.75 (m, 2H), 2.34-2.31 (m, 1H), 2.16-2.09 (m, 7H), 1.72-1.56 (m, 2H) |

### INTERMEDIATE O48: assumed trans-1-methyl-4-[2-[(2S)-pentan-2-ylamino]-5-[4-(piperazin-1-ylmethyl)phenyl]pyrrolo[2,3-d]pyrimidin-7-yl]cyclohexan-1-ol and Intermediate O49: assumed cis-1-methyl-4-[2-[(2S)-pentan-2-ylamino]-5-[4-(piperazin-1-ylmethyl)phenyl]pyrrolo[2,3-d]pyrimidin-7-yl]cyclohexan-1-ol

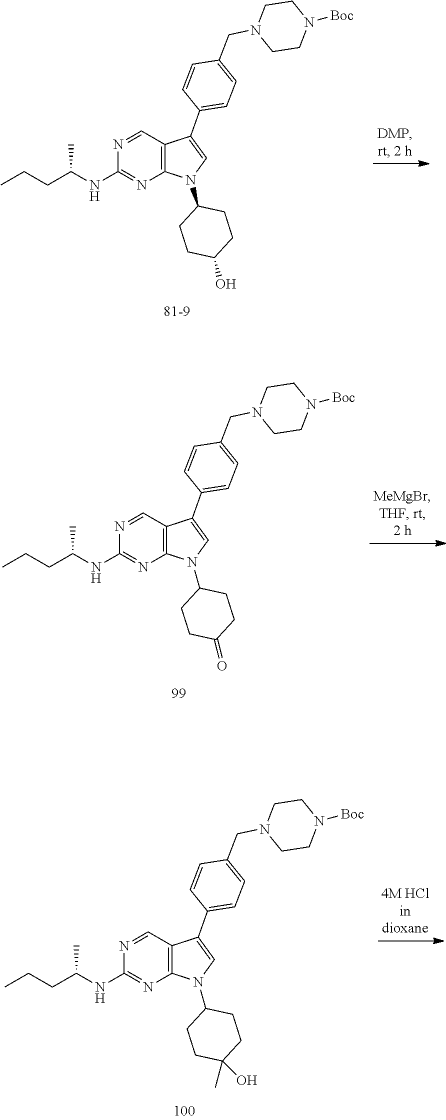 [SMILES: CCC[C@H](C)Nc1ncc2c(-c3ccc(CN4CCN(C(=O)OC(C)(C)C)CC4)cc3)cn([C@H]3CC[C@H](O)CC3)c2n1 ; CCC[C@H](C)Nc1ncc2c(-c3ccc(CN4CCN(C(=O)OC(C)(C)C)CC4)cc3)cn(C3CCC(=O)CC3)c2n1 ; CCC[C@H](C)Nc1ncc2c(-c3ccc(CN4CCN(C(=O)OC(C)(C)C)CC4)cc3)cn(C3CCC(C)(O)CC3)c2n1]

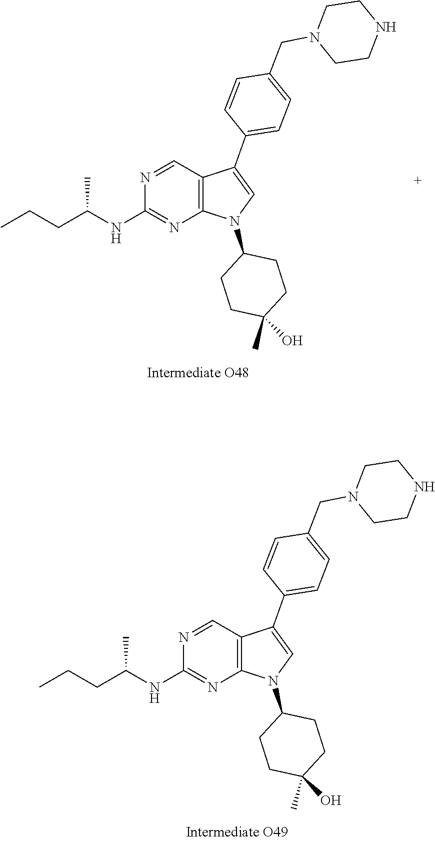 [SMILES: CCC[C@H](C)Nc1ncc2c(-c3ccc(CN4CCNCC4)cc3)cn([C@H]3CC[C@](C)(O)CC3)c2n1 ; CCC[C@H](C)Nc1ncc2c(-c3ccc(CN4CCNCC4)cc3)cn([C@H]3CC[C@@](C)(O)CC3)c2n1]

### Step 1: tert-Butyl 4-([4-[7-(4-oxocyclohexyl)-2-[(2S)-pentan-2-ylamino]pyrrolo[2,3-d]pyrimidin-5-yl]phenyl]methyl)piperazine-1-carboxylate

To a stirred solution of tert-butyl-4-[(4-[2-[(2S)-pentan-2-ylamino]-7-[trans-4-hydroxycyclohexyl]pyrrolo[2,3-d]pyrimidin-5-yl]phenyl)methyl]piperazine-1-carboxylate (600 mg, 1.04 mmol) in DCM (20.0 mL) was added Dess-Martin reagent (662 mg, 1.56 mmol) at 0° C. under nitrogen atmosphere. The resulting mixture was stirred for 2 h at room temperature under nitrogen atmosphere. The resulting mixture was concentrated under vacuum. The residue was purified by silica gel column chromatography, eluted with 50% ethyl acetate in petroleum ether to afford tert-butyl 4-([4-[7-(4-oxocyclohexyl)-2-[(2S)-pentan-2-ylamino]pyrrolo[2,3-d]pyrimidin-5-yl]phenyl]methyl)piperazine-1-carboxylate (400 mg, 67%) as a white solid. ¹H NMR (400 MHz, CD₃OD) δ 8.72 (d, J=9.3 Hz, 1H), 7.65-7.58 (m, 2H), 7.46-7.34 (m, 3H), 4.19-4.11 (m, 1H), 3.58 (s, 2H), 3.56-3.31 (m, 3H), 2.75 (s, 1H), 2.51 (d, J=14.2 Hz, 1H), 2.49-2.40 (m, 3H), 2.38 (s, 1H), 2.23-2.11 (m, 1H), 1.66 (s, 1H), 1.59-1.45 (m, 2H), 1.47 (s, 9H), 1.33-1.23 (m, 3H), 1.03-0.95 (m, 3H). LC/MS (ESI, m/z): [(M+1)]⁺=575.40

### Step 2: tert-Butyl 4-([4-[7-(4-hydroxy-4-methylcyclohexyl)-2-[(2S)-pentan-2-ylamino]pyrrolo[2,3-d]pyrimidin-5-yl]phenyl]methyl)piperazine-1-carboxylate

To a stirred solution of tert-butyl-4-([4-[7-(4-oxocyclohexyl)-2-[(2S)-pentan-2-ylamino]pyrrolo[2,3-d]pyrimidin-5-yl]phenyl]methyl)piperazine-1-carboxylate (400 mg, 0.70 mmol) in THF (15.0 mL) was added MeMgBr (4.00 mL, 4.03 mmol, 1 Min THF) dropwise at 0° C. under nitrogen atmosphere. After stirring for additional 2 h at room temperature, the resulting solution was concentrated under reduced pressure and the residue was diluted with water (10.0 mL). The mixture was extracted with DCM (3×50.0 mL). The combined organic phase was washed with brine (10.0 mL), dried over Na₂SO₄ and filtered. The filtrate was concentrated under reduced pressure. The residue was purified by silica gel column chromatography, eluted with 50% ethyl acetate in petroleum ether to afford tert-butyl-4-([4-[7-(4-hydroxy-4-methylcyclohexyl)-2-[(2S)-pentan-2-ylamino]pyrrolo[2,3-d]pyrimidin-5-yl]phenyl]methyl)piperazine-1-carboxylate (300 mg, 73%) as a yellow solid. ¹H NMR (400 MHz, CD₃OD) δ 8.71 (d, J=1.5 Hz, 1H), 7.65-7.56 (m, 2H), 7.43-7.32 (m, 3H), 4.59-4.45 (m, 1H), 4.19-4.05 (m, 2H), 3.55 (d, J=2.0 Hz, 2H), 3.49-3.32 (m, 4H), 2.49-2.31 (m, 4H), 2.31-2.15 (m, 2H), 2.03 (s, 1H), 1.89-1.75 (m, 3H), 1.79-1.62 (m, 2H), 1.69-1.64 (m, 1H), 1.64-1.58 (m, 1H), 1.52 (d, J=14.2 Hz, 1H), 1.47 (s, 9H), 1.42 (d, J=7.6 Hz, 1H), 1.32-1.21 (m, 7H), 1.09-0.92 (m, 4H). LC/MS (ESI, m/z): [(M+1)]⁺=591.40

### Step 3: Intermeidate O48: assumed trans-1-methyl-4-[2-[(2S)-pentan-2-ylamino]-5-[4-(piperazin-1-ylmethyl)phenyl]pyrrolo[2,3-d]pyrimidin-7-yl]cyclohexan-1-ol and Intermeidate O49: assumed cis-1-Methyl-4-[2-[(2S)-pentan-2-ylamino]-5-[4-(piperazin-1-ylmethyl)phenyl]pyrrolo[2,3-d]pyrimidin-7-yl]cyclohexan-1-ol

To a stirred solution of tert-butyl-4-([4-[7-(4-hydroxy-4-methylcyclohexyl)-2-[(2S)-pentan-2-ylamino]pyrrolo[2,3-d]pyrimidin-5-yl]phenyl] methyl)piperazine-1-carboxylate (250 mg, 0.43 mmol) in DCM (5.00 mL) was added HCl (4 M in 1,4-dioxane, 5.00 mL) dropwise at room temperature under nitrogen atmosphere. The resulting solution was stirred for 2 h at room temperature. The resulting solution was concentrated under reduced pressure. The residue was purified by reversed phase flash chromatography with the following conditions: Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 35% B -55% B gradient in 20 min; Detector: UV 220/254 nm. The fractions containing the desired product were collected at 52% B and concentrated under reduced pressure to afford assumed trans-1-methyl-4-[2-[(2S)-pentan-2-ylamino]-5-[4-(piperazin-1-ylmethyl)phenyl]pyrrolo[2,3-d]pyrimidin-7-yl]cyclohexan-1-ol (50 mg, 24%) as a white solid and assumed cis-1-methyl-4-[2-[(2S)-pentan-2-ylamino]-5-[4-(piperazin-1-ylmethyl)phenyl]pyrrolo[2,3-d]pyrimidin-7-yl]cyclohexan-1-ol (60 mg, 29%) as a white solid.

O48: ¹H NMR (400 MHz, CD₃OD) δ 8.71 (s, 1H), 7.65-7.58 (m, 2H), 7.44-7.37 (m, 3H), 4.57-4.46 (m, 1H), 4.14 (p, J=6.6 Hz, 1H), 3.57 (s, 2H), 2.99-2.85 (m, 4H), 2.59-2.51 (m, 3H), 2.23 (p, J=12.6 Hz, 2H), 1.99 (d, J=13.0 Hz, 2H), 1.91-1.82 (m, 2H), 1.76 (t, J=13.3 Hz, 2H), 1.69-1.60 (m, 1H), 1.60-1.44 (m, 5H), 1.26 (d, J=6.5 Hz, 3H), 0.99 (t, J=7.2 Hz, 3H). LC/MS (ESI, m/z): [(M+1)]⁺=491.35

O49: ¹H NMR (400 MHz, CD₃OD) δ 8.71 (s, 1H), 8.45 (s, 1H), 7.66-7.59 (m, 2H), 7.46-7.38 (m, 3H), 4.58 (t, J=12.5 Hz, 1H), 4.19-4.05 (m, 1H), 3.66 (s, 2H), 3.29-3.12 (m, 4H), 2.83-2.71 (m, 4H), 2.34-2.18 (m, 2H), 1.96-1.72 (m, 4H), 1.73-1.60 (m, 3H), 1.60-1.40 (m, 3H), 1.33-1.23 (m, 6H), 0.99 (t, J=7.1 Hz, 3H). LC/MS (ESI, m/z): [(M+1)]⁺=491.35

### INTERMEDIATE P1: 6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoic acid

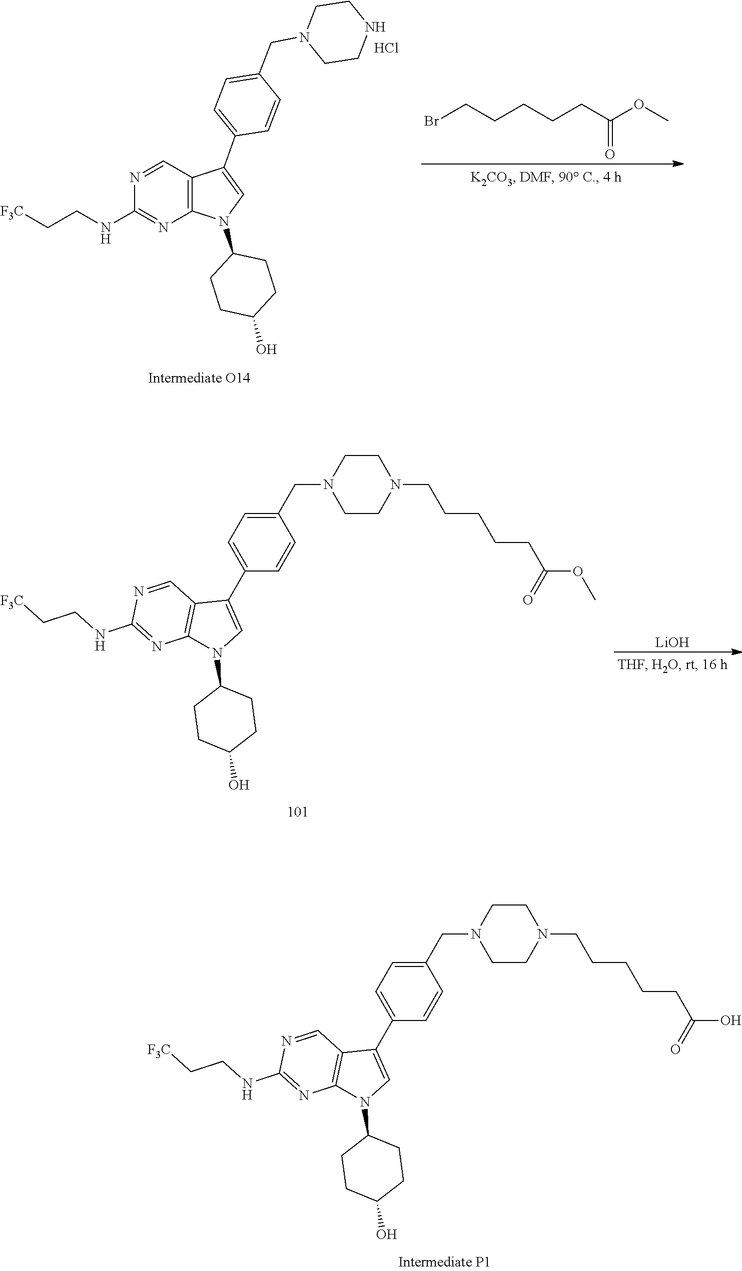

### Step 1: 6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoate

To a stirred solution of trans-4-[5-[4-(piperazin-1-ylmethyl)phenyl]-2-[(3,3,3-trifluoropropyl)amino]pyrrolo[2,3-d]pyrimidin-7-yl]cyclohexan-1-ol hydrochloride (100 mg, 0.19 mmol) and methyl 6-bromohexanoate (58.2 mg, 0.28 mmol) in DMF (3.00 mL) was added K₂CO₃ (51.3 mg, 0.37 mmol) at room temperature under nitrogen atmosphere. The resulting mixture was stirred for 4 h at 90° C. under nitrogen atmosphere. The resulting mixture was cooled down to room temperature and filtered. The filtrate was purified by reversed phase flash with the following conditions (Column: Spherical C¹⁸, 20˜40 um, 120 g; Mobile Phase A: water (plus 10 mM NH₄HCO₃), Mobile Phase B: ACN; Flow rate: 60 mL/min; Gradient (B %): 5%˜5%, 4 min; 35%˜60%, 20 min; 95%, 5 min; Detector: UV 220/254 nm; desired fractions were collected at 43% B) and concentrated under reduced pressure to afford methyl-6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoate (60.0 mg, 51%) as a light yellow solid. ¹HNMR (400 MHz, DMSO-d₆) δ 8.88 (s, 1H), 7.69-7.55 (m, 3H), 7.30 (d, J=7.9 Hz, 2H), 7.05 (t, J=5.9 Hz, 1H), 4.70 (d, J=4.3 Hz, 1H), 4.49-4.40 (m, 1H), 3.68-3.62 (m, 4H), 3.62-3.49 (m, 2H), 3.33 (s, 1H), 2.69-2.51 (m, 2H), 2.46-2.31 (m, 7H), 2.29 (t, J=7.4 Hz, 2H), 2.22 (t, J=7.3 Hz, 2H), 2.04-1.86 (m, 6H), 1.52 (p, J=7.5 Hz, 2H), 1.45-1.30 (m, 4H), 1.29-1.15 (m, 2H). LC/MS (ESI, m/z): [(M+1)]⁺=631.25

### Step 2: 6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoic acid

To a stirred mixture of methyl 6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoate (60.0 mg, 0.10 mmol) in THF (2.00 mL) was added a solution of LiOH (16.0 mg, 0.67 mmol) in H₂O (2.00 mL) dropwise at 0° C. The resulting mixture was stirred for 16 h at room temperature. The mixture was acidified to pH 2 with HCl (1 M). The resulting mixture was diluted with water (10.0 mL) and extracted with CH₂Cl₂ (3×10.0 mL). The combined organic layers was concentrated under reduced pressure and the residue was purified by reversed phase flash chromatography with the following conditions (Column: Spherical C¹⁸ Column, 20-40 um, 120 g; Mobile Phase A: water (plus 0.1% FA), Mobile Phase B: ACN; Flow rate: 60 mL/min; Gradient: 35% B to 50% B in 25 min; detector: UV 220/254 nm. The fractions containing the desired product were collected at 40% B) and concentrated under reduced pressure to afford 6-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]hexanoic acid (53.0 mg, 90%) as a light yellow solid. ¹HNMR (400 MHz, DMSO-d₆) δ 11.96 (s, 1H), 8.88 (s, 1H), 7.63 (d, J=7.5 Hz, 3H), 7.31 (d, J=7.9 Hz, 2H), 7.05 (t, J=5.9 Hz, 1H), 4.70 (s, 1H), 4.49-4.32 (m, 1H), 3.60-3.49 (m, 1H), 3.45 (s, 2H), 2.69-2.52 (m, 2H), 2.40-2.35 (m, 8H), 2.29-2.15 (m, 4H), 2.08 (s, 1H), 2.02-1.85 (m, 7H), 1.56-1.19 (m, 8H). LC/MS (ESI, m/z): [(M+1)]⁺=617.30

### INTERMEDIATE P2: [2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]acetic acid

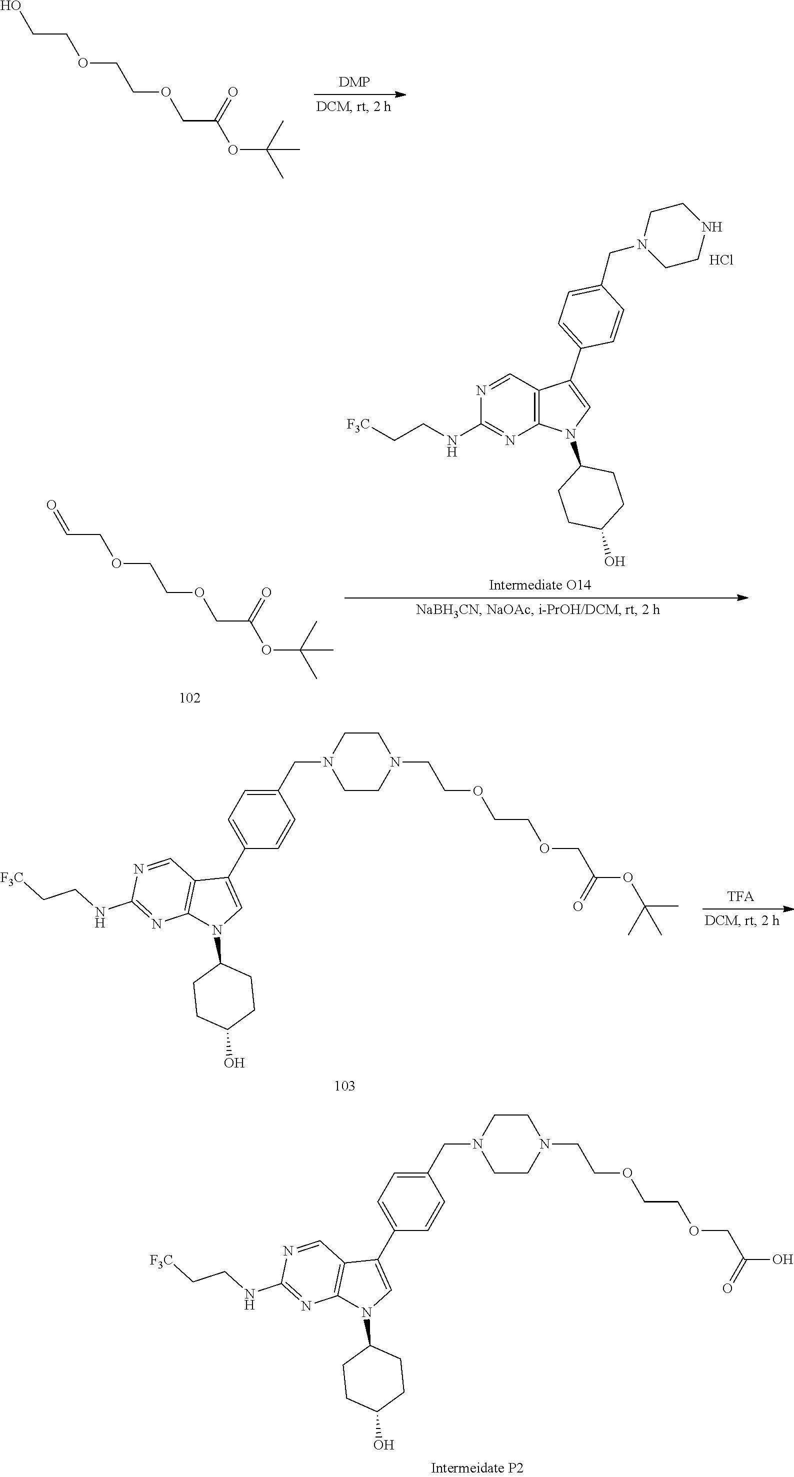 [SMILES: CC(C)(C)OC(=O)COCCOCCO ; CC(C)(C)OC(=O)COCCOCC=O ; O[C@H]1CC[C@H](n2cc(-c3ccc(CN4CCNCC4)cc3)c3cnc(NCCC(F)(F)F)nc32)CC1 ; Cl ; CC(C)(C)OC(=O)COCCOCCN1CCN(Cc2ccc(-c3cn([C@H]4CC[C@H](O)CC4)c4nc(NCCC(F)(F)F)ncc34)cc2)CC1 ; O=C(O)COCCOCCN1CCN(Cc2ccc(-c3cn([C@H]4CC[C@H](O)CC4)c4nc(NCCC(F)(F)F)ncc34)cc2)CC1]

### Step 1: tert-butyl 2-[2-(2-oxoethoxy)ethoxy]acetate

To a stirred solution of tert-butyl 2-[2-(2-hydroxyethoxy)ethoxy]acetate (94.0 mg, 0.43 mmol) in DCM (2.00 mL) was added Dess-Martin reagent (217 mg, 0.51 mmol) in portions at 0° C. under nitrogen atmosphere. The resulting mixture was stirred for 2 h at room temperature. The resulting mixture was filtered. The filter cake was washed with CH₂Cl₂ (4×10.0 mL). The combined filtrate was concentrated under reduced pressure. This resulted in tert-butyl 2-[2-(2-oxoethoxy)ethoxy]acetate (90.0 mg, 97%) as a white solid. ¹HNMR (400 MHz, CD₃OD) δ 9.72 (t, J=4.9 Hz, 1H), 4.05 (d, J=2.1 Hz, 2H), 3.69 (s, 2H), 3.77-3.63 (m, 2H), 3.39-3.31 (m, 2H), 1.50 (s, 9H). LC/MS (ESI, m/z): [(M+1)]⁺=219

### Step 2: tert-butyl-2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]acetate

To a stirred solution of trans-4-[5-[4-(piperazin-1-ylmethyl)phenyl]-2-[(3,3,3-trifluoropropyl)amino]pyrrolo[2,3-d]pyrimidin-7-yl]cyclohexan-1-ol hydrochloride (223 mg, 0.41 mmol) and tert-butyl 2-[2-(2-oxoethoxy)ethoxy]acetate (90.0 mg, 0.41 mmol) in MeOH (4.00 mL) was added KOAc (162 mg, 1.66 mmol) at room temperature under nitrogen atmosphere. After stirring for additional 20 min, NaBH₃CN (52.0 mg, 0.83 mmol) was added in portions over 1 min at 0° C. The resulting mixture was stirred for additional 2 h at room temperature. The resulting mixture was concentrated under reduced pressure. The residue was purified by reversed phase flash chromatography with the following conditions (Column: Spherical C¹⁸, 20˜40 um, 120 g; Mobile Phase A: water (plus 10 mM NH₄HCO₃), Mobile Phase B: ACN; Flow rate: 60 mL/min; Gradient (B %): 5%˜5%, 4 min; 35%˜60%, 20 min; 95%, 5 min; Detector: UV 220/254 nm; desired fractions were collected at 43% B and concentrated under reduced pressure to afford tert-butyl-2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]acetate (100 mg, 34%) as a white solid. ¹HNMR (400 MHz, DMSO-d6) δ 8.87 (s, 1H), 7.66-7.59 (m, 3H), 7.31 (d, J=8.0 Hz, 2H), 7.03 (t, J=5.9 Hz, 1H), 4.43 (s, 1H), 3.99 (d, J=6.5 Hz, 3H), 3.77-3.64 (m, 2H), 3.63-3.54 (m, 7H), 3.54-3.47 (m, 4H), 3.45 (s, 2H), 2.69-2.55 (m, 2H), 2.46 (t, J=6.0 Hz, 2H), 2.49-2.25 (m, 6H), 2.05-1.82 (m, 6H), 1.42 (s, 9H). LC/MS (ESI, m/z): [(M+1)]⁺=705.75

### Step 3: [2-(2-[4-[(4-[7-[trans-4-hydroxycyclohexyl]-2-[(3,3,3-trifluoropropyl)amino]pyrrolo[2,3-d]pyrimidin-5-y]phenyl)methyl]piperazin-1-yl]ethoxy)ethoxy]acetic acid

To a stirred solution of tert-butyl-2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]acetate (100 mg, 0.14 mmol) in DCM (8.00 mL) was added TFA (2.00 mL) dropwise at 0° C. under nitrogen atmosphere. The resulting mixture was stirred for 2 h at room temperature. The resulting mixture was concentrated under reduced pressure. The residue was purified by reversed phase flash chromatography with the following conditions (Column: Spherical C¹⁸, 20˜40 um, 120 g; Mobile Phase A: water (plus 0.05% HOAc), Mobile Phase B: ACN; Flow rate: 50 mL/min; Gradient (B %): 18%˜35%, 25 min; 95%, 5 min; Detector: UV 220/254 nm; The fractions containing the desired product were collected at 23% B and concentrated under reduced pressure to afford [2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]acetic acid (40.0 mg, 43%) as a white solid. ¹H NMR (400 MHz, CDCl₃) δ 8.71 (s, 1H), 7.52 (d, J=7.8 Hz, 2H), 7.38 (d, J=7.8 Hz, 2H), 7.28 (s, 2H), 7.13 (s, 1H), 4.07 (s, 2H), 3.85 (t, J=4.8 Hz, 2H), 3.78 (s, 2H), 3.79-3.73 (m, 2H), 3.73-3.71 (m, 2H), 3.71-3.64 (m, 4H), 3.27-3.08 (m, 4H), 3.05 (t, J=4.7 Hz, 2H), 2.94-2.84 (m, 4H), 2.59-2.45 (m, 2H), 2.25-2.12 (m, 4H), 2.19-2.02 (m, 4H). LC/MS (ESI, m/z): [(M+1)]⁺=649.65.

The following intermediates in Table 14 were synthesized according to the above two procedures:

TABLE 14

| Inter- | | | | |
|---|---|---|---|---|
| medi- | | | [(M + | |
| ate | Structure | Name | 1)]⁺ | ¹H NMR |
| P3 | 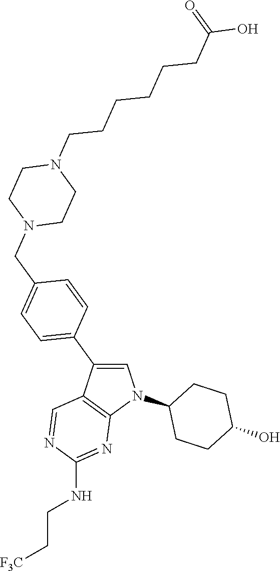 [SMILES: O=C(O)CCCCCCN1CCN(Cc2ccc(-c3cn([C@H]4CC[C@H](O)CC4)c4nc(NCCC(F)(F)F)ncc34)cc2)CC1] | 7-[4-[(4-[7- [trans-4- hydroxycyclohexyl]- 2-[(3,3,3- trifluoropropyl) amino]pyrrolo[2,3- d]pyrimidin-5- yl]phenyl)methyl] piperazin-1- yl]heptanoic acid | 631.25 | (400 MHz, DMSO-d₆) δ 11.86 (s, 1H), 8.88 (s, 1H), 7.63 (d, J = 7.6 Hz, 3H), 7.31 (d, J = 7.8 Hz, 2H), 7.05 (t, J = 5.9 Hz, 1H), 4.70 (s, 1H), 4.49-4.31 (m, 1H), 3.59-3.42 (m, 3H), 2.68-2.57 (m, 1H), 2.60- 2.48 (m, 5H), 2.27 (t, J = 7.4 Hz, 2H), 2.18 (t, J = 7.3 Hz, 2H), 2.05- 1.85 (m, 7H), 1.58-1.45 (m, 2H), 1.43-1.37 (m, 3H), 1.37-1.29 (m, 1H), 1.29-1.22 (m, 4H) |
| P4 | 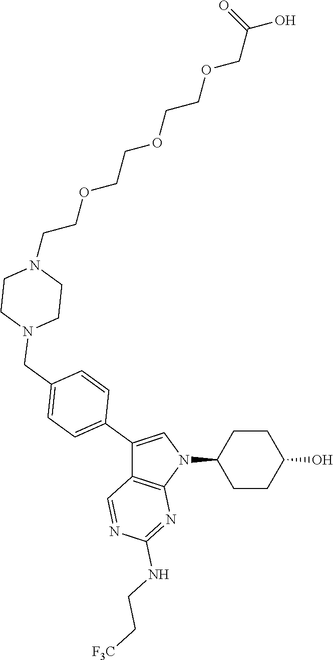 [SMILES: O=C(O)COCCOCCOCCN1CCN(Cc2ccc(-c3cn([C@H]4CC[C@H](O)CC4)c4nc(NCCC(F)(F)F)ncc34)cc2)CC1] | [2-[2-(2-[4-[(4- [7-[trans-4- hydroxycyclohexyl]- 2-[(3,3,3- trifluoropropyl) amino]pyrrolo[2,3- d]pyrimidin-5- yl]phenyl)methyl] piperazin-1- yl]ethoxy)ethoxy] ethoxy]acetic acid | 693.40 | (400 MHz, DMSO-d₆) δ 11.68 (s, 1H), 9.11 (s, 1H), 8.11 (s, 1H), 7.82 (d, J = 7.9 Hz, 1H), 7.71 (s, 1H), 7.37 (s, 1H), 7.24 (s, 1H), 7.12 (s, 1H), 4.47 (m, 1H), 4.37 (s, 1H), 4.09-4.00 (m, 2H), 3.89-3.75 (m, 2H), 3.79-3.62 (m, 2H), 3.63- 3.48 (m, 18H), 3.39-3.31 (m, 2H), 2.75-2.60 (m, 2H), 2.04-1.91 (m, 6H), 1.53-1.03 (m, 2H) |
| P5 | 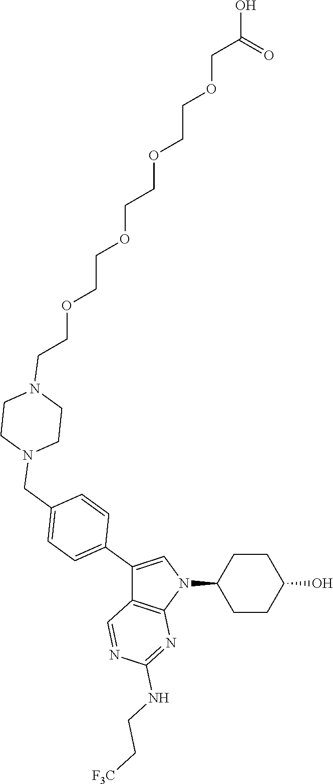 [SMILES: O=C(O)COCCOCCOCCOCCN1CCN(Cc2ccc(-c3cn([C@H]4CC[C@H](O)CC4)c4nc(NCCC(F)(F)F)ncc34)cc2)CC1] | 14-[4-[(4-[7- [trans-4- hydroxycyclohexyl]- 2-[(3,3,3- trifluoropropyl) amino]pyrrolo[2,3- d]pyrimidin-5- yl]phenyl)methyl] piperazin-1-yl]- 3,6,9,12- tetraoxatetra- decanoic acid | 736.40 | (400 MHz, CD₃OD) δ 8.76 (s, 1H), 7.62 (d, J = 7.8 Hz, 2H), 7.49- 7.38 (m, 3H), 4.18 (s, 1H), 4.16- 4.10 (m, 1H), 3.74-3.58 (m, 18H), 2.69-2.53 (m, 6H), 2.39-2.25 (m, 2H), 2.17-2.05 (m, 6H), 1.56 (q, J = 10.0 Hz, 2H) |

### INTERMEDIATE Q: 3-(5-bromo-3-methyl-2-oxo-2,3-dihydro-1H-1,3-benzodiazol-1-yl)-1-methylpiperidine-2,6-dione

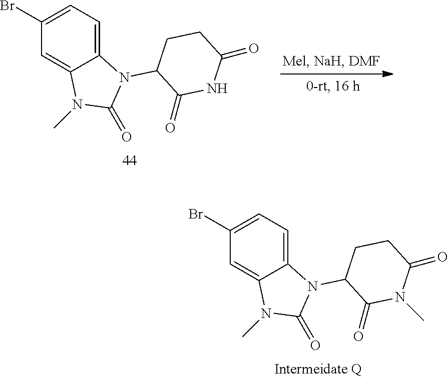 [SMILES: Cn1c(=O)n(C2CCC(=O)NC2=O)c2ccc(Br)cc21 ; CN1C(=O)CCC(n2c(=O)n(C)c3cc(Br)ccc32)C1=O]

A solution of 3-(5-bromo-3-methyl-2-oxo-1,3-benzodiazol-1-yl)piperidine-2,6-dione (5.00 g, 14.8 mmol) in DMF (80.0 mL) was treated with NaH (60% dispersion in mineral oil, 710 mg, 17.7 mmol) for 30 min at 0° C. under nitrogen atmosphere followed by the addition of Mel (2.52 g, 17.7 mmol). The resulting mixture was stirred for 16 h at room temperature under nitrogen atmosphere. The mixture was quenched with AcOH (1 mL) at 0° C. and diluted with water (200 mL). The mixture was extracted with EtOAc (3×200 mL). The combined organic layer was washed with brine (300 mL) and dried over anhydrous Na₂SO₄. After filtration, the filtrate was concentrated under reduced pressure. The residue was purified by reversed phase flash chromatography with the following conditions: Column: WelFlash™ C18-I, 20-40 um, 330 g; Eluent A: water (plus 10 mmol/L AcOH); Eluent B: ACN; Gradient: 30%-50% B in 25 min; Flow rate: 80 mL/min; Detector: UV 220/254 nm; desired fractions were collected at 43% B and concentrated under reduced pressure to afford 3-(5-bromo-3-methyl-2-oxo-1,3-benzodiazol-1-yl)-1-methylpiperidine-2,6-dione (1.70 g, 33%) as a light brown solid. ¹H NMR (400 MHz, DMSO-d₆) δ 7.48 (d, J=1.9 Hz, 1H), 7.21 (dd, J=8.4, 1.9 Hz, 1H), 7.12 (d, J=8.4 Hz, 1H), 5.45 (dd, J=13.0, 5.3 Hz, 1H), 3.35 (s, 3H), 3.03 (s, 3H), 3.01-2.91 (m, 1H), 2.84-2.63 (m, 2H), 2.04 (m, 1H). LC/MS (ESI, m/z): [(M+1)]⁺=352.10, 354.10

### INTERMEDIATE R1: tert-butyl 4-(14-[[1-(2,6-dioxopiperidin-3-yl)-3-methyl-2-oxo-2,3-dihydro-1H-1,3-benzodiazol-5-yl]amino]-3,6,9,12-tetraoxatetradecan-1-yl)piperazine-1-carboxylate

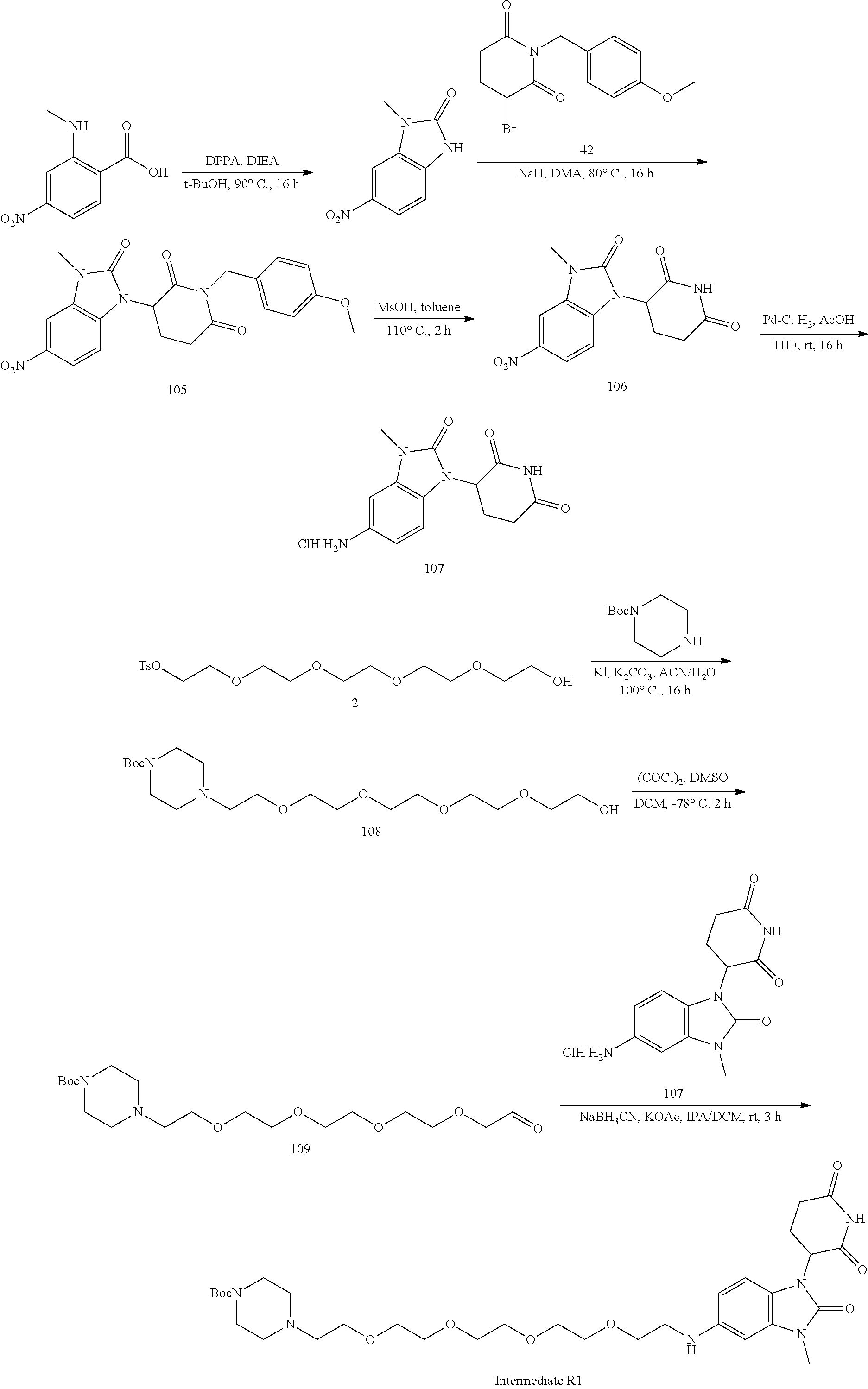

### Step 1: 1-methyl-6-nitro-2,3-dihydro-1H-1,3-benzodiazol-2-one

To a stirred solution of 2-(methylamino)-4-nitrobenzoic acid (33.7 g, 172 mmol) and DIEA (66.6 g, 516 mmol) in t-BuOH (200 mL) was added DPPA (56.7 g, 206 mmol) at room temperature under nitrogen atmosphere. The resulting mixture was stirred for 16 h at 90° C. The mixture was cooled down to room temperature and concentrated under reduced pressure. The residue was triturated with water (500 mL) and filtered. The filter cake was washed with water (3×100 mL). The crude product was purified by reversed phase flash chromatography with the following conditions: Column: WelFlash™ C18-I, 20-40 330 g; Eluent A: water (plus 10 mmol/L FA); Eluent B: ACN; Gradient: 35%-55% B in 15 min; Flow rate: 80 mL/min; Detector: UV 220/254 nm; desired fractions were collected at 40% B and concentrated under reduced pressure to afford 1-methyl-6-nitro-2,3-dihydro-1H-1,3-benzodiazol-2-one (29.0 g, 80%) as a light yellow solid. ¹H NMR (400 MHz, DMSO-d₆) δ 11.62 (s, 1H), 7.96 (d, J=8.5 Hz, 1H), 7.94 (s, 1H), 7.12 (d, J=8.5 Hz, 1H), 3.36 (s, 3H). LC/MS (ESI, m/z): [(M+1)]⁺=194.15.

### Step 2: 1-[(4-methoxyphenyl)methyl]-3-(3-methyl-5-nitro-2-oxo-2,3-dihydro-1H-1,3-benzodiazol-1-yl)piperidine-2,6-dione

To a solution of 1-methyl-6-nitro-2,3-dihydro-1H-1,3-benzodiazol-2-one (5.50 g, 28.5 mmol) in DMF (80.0 mL) was added sodium hydride (2.05 g, 51.3 mmol, 60% dispersion in mineral oil) at 0° C. After stirring for additional 15 minutes, a solution of 3-bromo-1-[(4-methoxyphenyl)methyl]piperidine-2,6-dione (6.84 g, 21.9 mmol) in DMF (50.0 mL) was added and the mixture was stirred for 16 h at room temperature. The mixture was acidified to pH 6 with acetic acid (2 mL). The resulting mixture was diluted with water (600 mL) and extracted with EtOAc (3×150 mL). The combined organic layers were washed with brine (200 mL) and dried over anhydrous Na₂SO₄. After filtration, the filtrate was concentrated under reduced pressure. The residue was purified by silica gel column chromatography, eluted with 30% ethyl acetate in petroleum ether to afford crude product. The crude product was further purified by reversed phase flash chromatography with the following conditions: Column: WelFlash™ C18-I, 20-40 μm, 330 g; Eluent A: water (plus 10 mmol/L FA); Eluent B: ACN; Gradient: 40%-60% B in 25 min; Flow rate: 80 mL/min; Detector: UV 220/254 nm; desired fractions were collected at 54% B and concentrated under reduced pressure to afford 1-[(4-methoxyphenyl)methyl]-3-(3-methyl-5-nitro-2-oxo-2,3-dihydro-1H-1,3-benzodiazol-1-yl)piperidine-2,6-dione (2.50 g, 21%) as a light yellow solid. ¹H NMR (400 MHz, DMSO-d₆) δ 8.14 (d, J=2.2 Hz, 1H), 8.03 (dd, J=8.7, 2.3 Hz, 1H), 7.30 (d, J=8.7 Hz, 1H), 7.25-7.18 (m, 2H), 6.92-6.81 (m, 2H), 5.69 (dd, J=12.9, 5.4 Hz, 1H), 4.81 (q, J=14.4 Hz, 2H), 3.73 (s, 3H), 3.46 (s, 3H), 3.11-3.14 (m, 1H), 2.92-2.70 (m, 2H), 2.14 (ddd, J=12.6, 5.6, 3.3 Hz, 1H). LC/MS (ESI, m/z): [(M+1)]⁺=425.25.

### Step 3: 3-(3-methyl-5-nitro-2-oxo-2,3-dihydro-1H-1,3-benzodiazol-1-yl)piperidine-2,6-dione

To a stirred mixture of 1-[(4-methoxyphenyl)methyl]-3-(3-methyl-5-nitro-2-oxo-2,3-dihydro-1H-1,3-benzodiazol-1-yl)piperidine-2,6-dione (5.00 g, 11.8 mmol, 1 equiv) in toluene (25.0 mL) was added methanesulfonic acid (25.0 mL, 385 mmol) at room temperature under nitrogen atmosphere. The resulting mixture was stirred for 2 h at 110° C. The mixture was cooled down to room temperature and concentrated under reduced pressure. The residue was diluted with ice-water (300 mL). The precipitated solids were collected by filtration and washed with water (3×20.0 mL). The crude product was purified by reversed phase flash chromatography with the following conditions: Column: WelFlash™ C18-I, 20-40 μm, 330 g; Eluent A: water (plus 10 mmol/L FA); Eluent B: ACN; Gradient: 40%-70% B in 30 min; Flow rate: 80 mL/min; Detector: UV 220/254 nm; desired fractions were collected at 58% B and concentrated under reduced pressure to afford 3-(3-methyl-5-nitro-2-oxo-2,3-dihydro-1H-1,3-benzodiazol-1-yl)piperidine-2,6-dione (1.70 g, 46%) as a light brown solid. ¹H NMR (400 MHz, DMSO-d₆) δ 11.22 (s, 1H), 8.15 (d, J=2.2 Hz, 1H), 8.07 (d, J=8.5 Hz, 1H), 7.39 (d, J=8.7 Hz, 1H), 5.53 (dd, J=12.9, 5.4 Hz, 1H), 3.46 (d, J=1.6 Hz, 3H), 2.91 (s, 1H), 2.75 (td, J=12.9, 4.2 Hz, 1H), 2.66 (d, J=18.3 Hz, 1H), 2.09 (dq, J=8.6, 4.8 Hz, 1H). LC/MS (ESI, m/z): [(M+1)]⁺=305.25.

### Step 4: 3-(5-amino-3-methyl-2-oxo-2,3-dihydro-1H-1,3-benzodiazol-1-yl)piperidine-2,6-dione hydrochloride

To a solution of 3-(3-methyl-5-nitro-2-oxo-2,3-dihydro-1H-1,3-benzodiazol-1-yl)piperidine-2,6-dione (1.40 g, 4.60 mmol) in AcOH (200 mL) was added 10% palladium on activated carbon (200 mg) under nitrogen atmosphere. The mixture was stirred for 16 h at room temperature under hydrogen atmosphere using a hydrogen balloon. After filtration, the filtrate was concentrated under reduced pressure. The residue was purified by reversed phase flash chromatography with the following conditions: Column: WelFlash™ C18-I, 20-40 μm, 330 g; Eluent A: water (plus 10 mmol/L HCl); Eluent B: ACN; Gradient: 5%-25% B in 20 min; Flow rate: 80 mL/min; Detector: UV 220/254 nm; desired fractions were collected at 12% B and concentrated under reduced pressure to afford 3-(5-amino-3-methyl-2-oxo-2,3-dihydro-1H-1,3-benzodiazol-1-yl)piperidine-2,6-dione hydrochloride (270 mg, 19%) as a white solid. ¹H NMR (400 MHz, CD₃OD) δ 7.31-7.26 (m, 2H), 7.19 (dd, J=8.3, 2.1 Hz, 1H), 5.41 (dd, J=12.9, 5.4 Hz, 1H), 3.49 (s, 3H), 2.99 (ddd, J=18.4, 13.7, 5.3 Hz, 1H), 2.91-2.74 (m, 2H), 2.30-2.20 (m, 1H). LC/MS (ESI, m/z): [(M+1)]⁺=275.25.

### Step 5: tert-butyl 4-(14-hydroxy-3,6,9,12-tetraoxatetradecan-1-yl)piperazine-1-carboxylate

To a stirred solution of 14-[(4-methylbenzenesulfonyl)oxy]-3,6,9,12-tetraoxatetradecan-1-ol (6.50 g, 16.6 mmol) and tert-butyl piperazine-1-carboxylate (4.63 g, 24.9 mmol) in ACN (30.0 mL) and H₂O (30.0 mL) were added KI (2.75 g, 16.6 mmol) and K₂CO₃ (4.58 g, 33.1 mmol) at room temperature under nitrogen atmosphere. The resulting mixture was stirred for 16 h at 100° C. The solution was cooled down to room temperature and concentrated under reduced pressure. The residue was purified by reversed phase flash chromatography with the following conditions: Column: WelFlash™ C18-I, 20-40 μm, 330 g; Eluent A: water (plus 10 mmol/L FA); Eluent B: ACN; Gradient: 15%-30% B in 20 min; Flow rate: 80 mL/min; Detector: UV 220/200 nm; desired fractions were collected at 20% B and concentrated under reduced pressure to afford tert-butyl 4-(14-hydroxy-3,6,9,12-tetraoxatetradecan-1-yl)piperazine-1-carboxylate (6.20 g, 87%) as a light yellow oil. ¹H NMR (400 MHz, DMSO-d₆) δ 3.55-3.46 (m, 18H), 3.44-3.38 (m, 4H), 2.52-2.50 (d, J=3.2 Hz, 2H), 2.40 (t, J=5.1 Hz, 4H), 1.39 (s, 9H). LC/MS (ESI, m/z): [(M+1)]⁺=407.40

### Step 6: tert-butyl 4-(14-oxo-3,6,9,12-tetraoxatetradecan-1-yl)piperazine-1-carboxylate

To a stirred solution of DMSO (1.05 mL, 13.4 mmol) in DCM (5.00 mL) was added oxalyl chloride (0.63 mL, 4.95 mmol) dropwise at −78° C. under nitrogen atmosphere. The above solution was stirred for 30 min at −78° C. under nitrogen atmosphere. To the above mixture was added a solution of tert-butyl 4-(14-hydroxy-3,6,9,12-tetraoxatetradecan-1-yl)piperazine-1-carboxylate (1.00 g, 2.46 mmol) in DCM (5.00 mL) dropwise at −78° C. The resulting mixture was stirred for additional 30 min at −78° C. To the above mixture was added TEA (4.10 mL, 29.5 mmol) at −78° C. The resulting mixture was stirred for additional 30 min at −78° C. and 2 h at room temperature. The resulting mixture was concentrated under reduced pressure. The residue was purified by reversed phase flash chromatography with the following conditions: Column: WelFlash™ C18-I, 20-40 μm, 120 g; Eluent A: water (plus 10 mmol/L FA); Eluent B: ACN; Gradient: 15%-40% B in 25 min; Flow rate: 50 mL/min; Detector: UV 220/200 nm; desired fractions were collected at 28% B and concentrated under reduced pressure to afford tert-butyl 4-(14-oxo-3,6,9,12-tetraoxatetradecan-1-yl)piperazine-1-carboxylate (700 mg, 70%) as a light yellow oil. ¹H NMR (400 MHz, DMSO-d₆) δ 8.16 (s, 1H), 3.57-3.46 (m, 14H), 3.35-3.26 (m, 4H), 2.54-2.48 (m, 4H), 2.42-2.38 (m, 4H), 1.39 (s, 9H). LC/MS (ESI, m/z): [(M+1)]⁺=405.60

The following intermediates in Table 15 were synthesized according to the above procedure:

TABLE 15

| Inter- | | | | |
|---|---|---|---|---|
| medi- | | | MS: | |
| ate | Structure | Name | [(M + 23)]⁺ | ¹H NMR |
| 110 | 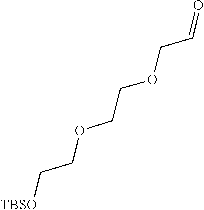 [SMILES: CC(C)(C)[Si](C)(C)OCCOCCOCC=O] | 2,2,3,3-tetramethyl- 4,7,10-trioxa-3- siladodecan-12-al | 285.25 | (400 MHz, DMSO-d₆) δ 9.58 (s, 1H), 3.73-3.29 (m, 10H), 0.87 (s, 9H), 0.06 (s, 6H) |

### Step 7: tert-butyl 4-(14-[[1-(2,6-dioxopiperidin-3-yl)-3-methyl-2-oxo-2,3-dihydro-1H-1,3-benzodiazol-5-yl]amino]-3,6,9,12-tetraoxatetradecan-1-yl)piperazine-1-carboxylate

To a stirred solution of tert-butyl 4-(14-oxo-3,6,9,12-tetraoxatetradecan-1-yl)piperazine-1-carboxylate (200 mg, 0.49 mmol) and KOAc (97.1 mg, 0.99 mmol) in IPA (6.00 mL) and DCM (0.50 mL) was added 3-(5-amino-3-methyl-2-oxo-2,3-dihydro-1H-1,3-benzodiazol-1-yl)piperidine-2,6-dione (136 mg, 0.49 mmol) at room temperature under nitrogen atmosphere. After stirring for additional 10 min, NaBH₃CN (62.1 mg, 0.99 mmol) was added. The resulting mixture was stirred for additional 3 h at room temperature. The resulting mixture was concentrated under reduced pressure. The residue was purified by reversed phase flash chromatography with the following conditions: Column: WelFlash™ C18-I, 20-40 um, 120 g; Eluent A: water (plus 10 mmol/L FA); Eluent B: ACN; Gradient: 35%-60% B in 25 min; Flow rate: 50 mL/min; Detector: UV 220/254 nm; desired fractions were collected at 55% B and concentrated under reduced pressure to afford tert-butyl 4-(14-[[1-(2,6-dioxopiperidin-3-yl)-3-methyl-2-oxo-2,3-dihydro-1H-1,3-benzodiazol-5-yl]amino]-3,6,9,12-tetraoxatetradecan-1-yl)piperazine-1-carboxylate (75.0 mg, 16%) as a grey solid. LC/MS (ESI, m/z): [(M+1)]⁺=663.40.

The following intermediates in Table 16 were synthesized according to the above procedure:

TABLE 16

| Inter- | | | MS: | |
|---|---|---|---|---|
| medi- | | | [(M + | |
| ate | Structure | Name | 1)]⁺ | ¹H NMR |
| 110 | 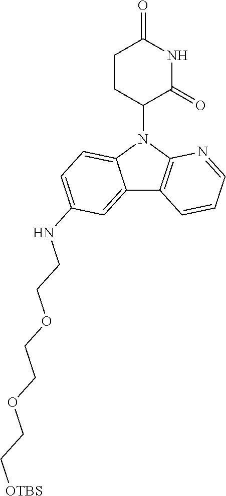 [SMILES: CC(C)(C)[Si](C)(C)OCCOCCOCCNc1ccc2c(c1)c1cccnc1n2C1CCC(=O)NC1=O] | 3-[6-(11, 11, 12, 12- tetramethyl-4,7,10- trioxa-1-aza-11- silatridecan-1-yl)- 9H-pyrido[2,3- b]indol-9- yl]piperidine-2,6- dione | 541.35 | (400 MHz, DMSO-d₆) δ 11.10 (s, 1H), 8.42 (m, 1H), 8.33 (m, 1H), 7.39-7.31 (m, 2H), 7.16 (m, 1H), 6.92 (m, 1H), 5.92 (s, 1H), 5.30 (t, J = 5.9 Hz, 1H), 3.67 (m, 4H), 3.58 (s, 4H), 3.47 (t, J = 5.1 Hz, 2H), 3.29 (m, 2H), 3.05-2.94 (m, 2H), 2.69 (d, J = 13.1 Hz, 1H), 0.86 (s, 9H), 0.04 (s, 6H) |

### INTERMEDIATE R2: tert-butyl 4-[trans-3-[2-[1-(2,6-dioxopiperidin-3-yl)-3-methyl-2-oxo-1,3-benzodiazol-5-yl]ethoxy]cyclobutyl]piperazine-1-carboxylate

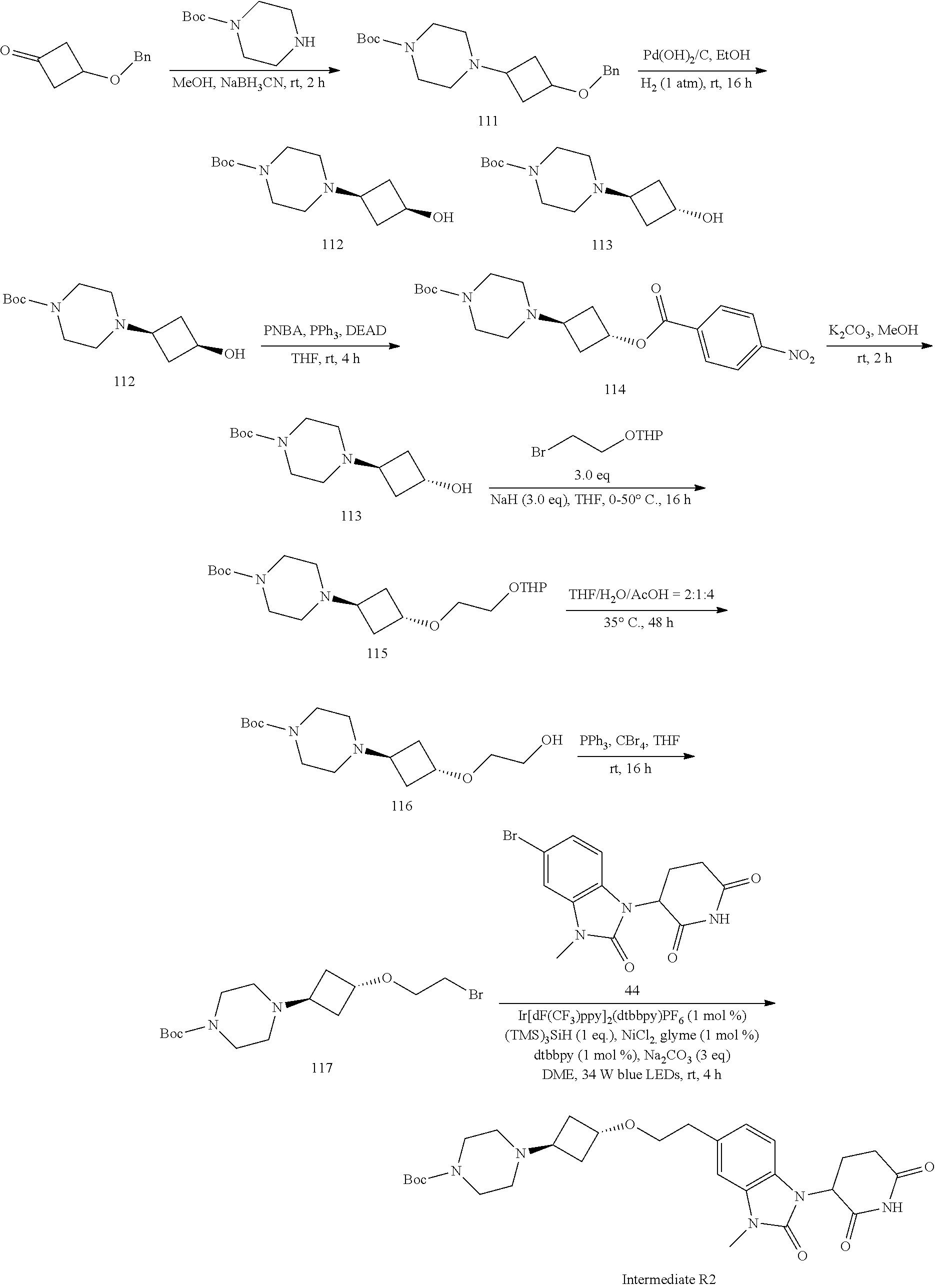

### Step 1: tert-butyl 4-[3-(benzyloxy)cyclobutyl]piperazine-1-carboxylate

To a stirred solution of tert-butyl piperazine-1-carboxylate (5.00 g, 26.9 mmol), 3-(benzyloxy)cyclobutan-1-one (4.73 g, 26.9 mmol) and CH₃COOH (8.06 g, 134 mmol) in CH₃OH (70.0 mL) was added NaBH₃CN (5.06 g, 80.5 mmol) at room temperature under nitrogen atmosphere. The resulting solution was stirred for 2 h at room temperature. The resulting solution was purified by reversed flash chromatography with the following conditions: Column: WelFlash™ C18-I, 20-40 um, 330 g; Eluent A: water (plus 10 mmol/L FA); Eluent B: ACN; Gradient: 20%-40% B in 20 min; Flow rate: 80 mL/min; Detector: UV 220/254 nm; desired fractions were collected at 55% B and concentrated under reduced pressure to afford tert-butyl 4-[3-(benzyloxy)cyclobutyl]piperazine-1-carboxylate (6.00 g, 64%) as a colorless oil. ¹H NMR (400 MHz, CD₃OD) δ 7.38-7.24 (m, 5H), 4.44 (s, 2H), 3.89-3.78 (m, 1H), 3.43 (t, J=5.1 Hz, 4H), 2.48 (m, 2H), 2.41-2.27 (m, 5H), 1.82 (m, 2H), 1.47 (s, 9H). LC/MS (ESI, m/z): [(M+1)]⁺=347.20

### Step 2: tert-butyl 4-[cis-3-hydroxycyclobutyl]piperazine-1-carboxylate and tert-butyl 4-[trans-3-hydroxycyclobutyl]piperazine-1-carboxylate

To a solution of tert-butyl 4-[3-(benzyloxy)cyclobutyl]piperazine-1-carboxylate (12.0 g, 34.6 mmol) in EtOH (30.0 mL) was added 10% Pd(OH)₂ on activated carbon (3.00 g) under nitrogen atmosphere. The mixture was stirred for 16 h at room temperature under hydrogen atmosphere using a hydrogen balloon. After filtration, the filtrate was concentrated under reduced pressure. The residue was purified by reversed phase flash chromatography with the following conditions: Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 50% B-65% B gradient in 20 min; Detector: UV 254/220 nm. The fractions containing the desired product were collected at 53% B and concentrated under reduced pressure to afford tert-butyl 4-[cis-3-hydroxycyclobutyl]piperazine-1-carboxylate (5.50 g, 62%) and tert-butyl 4-[trans-3-hydroxycyclobutyl]piperazine-1-carboxylate (0.50 g, 6%) as a white solid.

112: ¹H NMR (400 MHz, CDCl₃) δ 4.02 (m, 1H), 3.43 (q, J=4.8 Hz, 4H), 2.54 (m, 2H), 2.37-2.21 (m, 5H), 1.80 (m, 2H), 1.50-1.43 (m, 9H). LC/MS (ESI, m/z): [(M+1)]⁺=257.25

113: ¹H NMR (400 MHz, CD₃OD) δ 4.34 (m, 1H), 3.45 (t, J=5.3 Hz, 4H), 3.05-2.93 (m, 1H), 2.34 (t, J=5.2 Hz, 4H), 2.25 (m, 2H), 2.05 (m, 2H), 1.47 (s, 9H). LC/MS (ESI, m/z): [(M+1)]⁺=257.10

### Step 3: tert-butyl 4-[trans-3-(4-nitrobenzoyloxy)cyclobutyl]piperazine-1-carboxylate

To a stirred solution of tert-butyl 4-[cis-3-hydroxycyclobutyl]piperazine-1-carboxylate (5.50 g, 21.5 mmol), PPh₃ (22.5 g, 85.8 mmol) and p-nitrobenzoic acid (14.4 g, 85.8 mmol) in THF (40.0 mL) was added DEAD (15.0 g, 85.8 mmol) dropwise at 0° C. under nitrogen atmosphere. The resulting solution was stirred for 4 h at room temperature and concentrated under vacuum. The residue was diluted with water (100 mL) and extracted with EtOAc (3×150 mL). The combined organic phase was washed with brine (100 mL), dried with Na₂SO₄ and filtered. The filtrate was concentrated under reduced pressure. The residue was purified by silica gel column chromatography, eluted with 10% methanol in dichloromethane to afford tert-butyl 4-[trans-3-(4-nitrobenzoyl oxy)cyclobutyl]piperazine-1-carboxylate (3.80 g, 44%) as a yellow solid.

¹H NMR (400 MHz, DMSO-d₆) δ 8.40-8.32 (m, 2H), 8.25-8.18 (m, 2H), 5.22 (m, 1H), 3.33 (d, J=10.3 Hz, 4H), 2.99 (m, 1H), 2.41 (m, 2H), 2.27 (m, 6H), 1.40 (s, 9H). LC/MS (ESI, m/z): [(M+1)]⁺=406.25

### Step 4: tert-butyl 4-[trans-3-hydroxycyclobutyl]piperazine-1-carboxylate

A mixture of tert-butyl 4-[trans-3-(4-nitrobenzoyloxy)cyclobutyl]piperazine-1-carboxylate (3.80 g, 9.37 mmol) and K₂CO₃ (3.89 g, 28.1 mmol) in MeOH (50.0 mL) was stirred for 2 h at room temperature. The resulting solution was concentrated under vacuum. The residue was diluted with water (50.0 mL) and extracted with EtOAc (3×50.0 mL). The combined organic phase was washed with brine (50.0 mL), dried with Na₂SO₄ and filtered. The filtrate was concentrated under reduced pressure. The residue was purified by silica gel column chromatography, eluted with 10% methanol in dichloromethane to afford tert-butyl 4-[trans-3-hydroxycyclobutyl]piperazine-1-carboxylate (1.60 g, 67%) as a white solid.

¹H NMR (400 MHz, CD₃OD) δ 4.34 (m, 1H), 3.45 (t, J=5.3 Hz, 4H), 3.05-2.93 (m, 1H), 2.34 (t, J=5.2 Hz, 4H), 2.25 (m, 2H), 2.05 (m, 2H), 1.47 (s, 9H) LC/MS (ESI, m/z): [(M+1)]⁺=257.10

### Step 5: tert-butyl 4-[tans-3-[2-(oxan-2-yloxy)ethoxy]cyclobutyl]piperazine-1-carboxylate

To a stirred solution of tert-butyl 4-[trans-3-hydroxycyclobutyl]piperazine-1-carboxylate (1.60 g, 6.24 mmol) in THF (50.0 mL) was added NaH (749 mg, 18.7 mmol, 60% dispersion in mineral oil) in portions at 0° C. under nitrogen atmosphere. The resulting mixture was stirred for additional 30 min at room temperature. To the above mixture was added 2-(2-bromoethoxy)oxane (3.92 g, 18.7 mmol) dropwise over at 0° C. The resulting mixture was stirred for additional 16 h at 50° C. The mixture was cooled down to 25° C., quenched by water (50.0 mL) and extracted with EtOAc (3×50.0 mL). The combined organic phase was washed with brine (50.0 mL), dried over anhydrous Na₂SO₄ and filtered. The filtrate was concentrated under reduced pressure. The residue was purified by reversed phase flash chromatography with the following conditions: Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 33% B-45% B gradient in 20 min; Detector: UV 220/254 nm. The fractions containing the desired product were collected at 40% B and concentrated under reduced pressure to afford tert-butyl 4-[trans-3-[2-(oxan-2-yloxy)ethoxy]cyclobutyl]piperazine-1-carboxylate (700 mg, 29%) as a white solid. ¹H NMR (400 MHz, DMSO-d₆) δ 4.58 (t, J=3.7 Hz, 1H), 3.99 (m, 1H), 3.80-3.64 (m, 2H), 3.45 (m, 4H), 3.31 (s, 2H), 2.83-2.72 (m, 1H), 2.19 (t, J=5.0 Hz, 4H), 2.13-2.02 (m, 2H), 1.96 (m, 2H), 1.71 (d, J=7.4 Hz, 1H), 1.61 (m, 1H), 1.53-1.42 (m, 4H), 1.39 (s, 9H). LC/MS (ESI, m/z): [(M+1)]⁺=385.35

The following intermediates in Table 17 were synthesized according to the above procedure:

TABLE 17

| Inter- | | | | |
|---|---|---|---|---|
| medi- | | | MS: | |
| ate | Structure | Name | [(M +1)]⁺ | ¹H NMR |
| 118 | 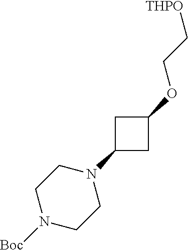 | tert-butyl 4-[cis- 3-[2-(oxan-2- yloxy)ethoxy]cyclo- butyl]piperazine- 1-carboxylate | 385.35 | (400 MHz, CDCl₃) δ 4.64 (m, 1H), 3.88 (m, 1H), 3.86-3.72 (m, 2H), 3.65-3.41 (m, 8H), 2.45 (m, 2H), 2.32 (m, 5H), 1.85 (m, 3H), 1.73 (m, 1H), 1.68-1.50 (m, 4H), 1.47 (s, 9H) |

### Step 6: tert-butyl 4-[trans-3-(2-hydroxyethoxy)cyclobuty]piperazine-1-carboxylate

A solution of tert-butyl 4-[trans-3-[2-(oxan-2-yloxy)ethoxy]cyclobutyl]piperazine-1-carboxylate (700 mg, 1.82 mmol) in AcOH (10.0 mL) and H₂O (1.00 mL) were stirred for 48 h at 35° C. under nitrogen atmosphere. The resulting mixture was concentrated under vacuum. The residue was purified by reversed phase flash chromatography with the following conditions: Column: Spherical C18, 20-40 um, 330 g; Mobile Phase A: water (plus 10 mM NH₄HCO₃); Mobile Phase B: ACN; Flow rate: 80 mL/min; Gradient: 5%-5% B, 10 min, 33% B -45% B gradient in 20 min; Detector: UV 220/200 nm. The fractions containing the desired product were collected at 40% B and concentrated under reduced pressure to afford tert-butyl 4-[trans-3-(2-hydroxyethoxy)cyclobutyl]piperazine-1-carboxylate (400 mg, 73%) as a white solid. ¹H NMR (400 MHz, CD₃OD) δ 4.11 (m, 1H), 3.67 (t, J=4.9 Hz, 2H), 3.45 (m, 6H), 2.97 (m, 1H), 2.34 (t, J=5.1 Hz, 4H), 2.25-2.11 (m, 4H), 1.47 (s, 9H). LC/MS (ESI, m/z): [(M+1)]⁺=301.20.

The following intermediates in Table 18 were synthesized according to the above procedure:

TABLE 18

| Inter- | | | MS: | |
|---|---|---|---|---|
| mediate | Structure | Name | [(M +1)]⁺ | ¹H NMR |
| 119 | 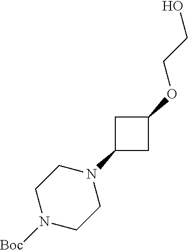 [SMILES: CC(C)(C)OC(=O)N1CCN([C@H]2C[C@@H](OCCO)C2)CC1] | tert-butyl 4-[cis- 3-(2- hydroxyethoxy) cyclobutyl] piperazine-1- carboxylate | 301.20 | (400 MHz, CDCl₃) δ 3.85-3.74 (m, 1H), 3.74-3.67 (m, 2H), 3.47 (m, 6H), 2.54-2.44 (m, 2H), 2.33- 2.27 (m, 4H), 2.10-2.00 (m, 1H), 1.86 (m, 2H), 1.47 (s, 9H) |

### Step 7: tert-Butyl 4-[trans-3-(2-bromoethoxy)cyclobutyl]piperazine-1-carboxylate

To a stirred solution of tert-butyl 4-[trans-3-(2-hydroxyethoxy)cyclobutyl]piperazine-1-carboxylate (150 mg, 0.50 mmol) in THF (20.0 mL) were added PPh₃ (262 mg, 1.00 mmol) and CBr₄ (331 mg, 1.00 mmol) at room temperature under nitrogen atmosphere. The resulting mixture was stirred for 16 h at room temperature. The resulting mixture was concentrated under vacuum. The residue was purified by reversed phase flash chromatography with the following conditions: Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 33% B-45% B gradient in 20 min; Detector: UV 220/200 nm. The fractions containing the desired product were collected at 40% B and concentrated under reduced pressure to afford tert-butyl 4-[trans-3-(2-bromoethoxy)cyclobutyl]piperazine-1-carboxylate (80.0 mg, 44%) as a white solid. ¹H NMR (400 MHz, DMSO-d₆) δ 4.04 (m, 1H), 3.66-3.53 (m, 4H), 2.82-2.77 (m, 1H), 2.20 (t, J=5.1 Hz, 4H), 2.13-2.08 (m, 2H), 2.01-1.97 (m, 2H), 1.40 (s, 9H). LC/MS (ESI, m/z): [(M+1)]⁺=363.25, 365.25.

The following intermediates in Table 19 were synthesized according to the above procedure:

TABLE 19

| Inter- | | | MS: | |
|---|---|---|---|---|
| mediate | Structure | Name | [(M +1)]⁺ | ¹H NMR |
| 120 | 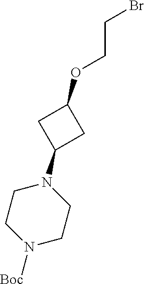 [SMILES: CC(C)(C)OC(=O)N1CCN([C@H]2C[C@@H](OCCBr)C2)CC1] | tert-butyl 4- [cis-3-(2- bromoethoxy) cyclobutyl] piperazine-1- carboxylate | 363.25, 362.25. | (400 MHz, CDCl₃) 5.60-5.54 (m, 2H), 3.81 (p, J = 7.0 Hz, 1H), 3.68 (t, J = 6.3 Hz, 2H), 3.58 (t, J = 5.2 Hz, 4H), 3 .43 (t, J = 6.2 Hz, 2H), 2.60 (t, J = 5.3 Hz, 4H), 2.54-2.51 (m, 1H), 2.20-2.16 (m, 2H), 1.46 (s, 9H) |

### Step 8: tert-butyl 4-[trans-3-[2-[1-(2,6-dioxopiperidin-3-yl)-3-methyl-2-oxo-1,3-benzodiazol-5-yl]ethoxy]cyclobutyl]piperazine-1-carboxylate

To a stirred mixture of tert-butyl 4-[trans-3-(2-bromoethoxy)cyclobutyl]piperazine-1-carboxylate (80.0 mg, 0.22 mmol), 3-(5-bromo-3-methyl-2-oxo-1,3-benzodiazol-1-yl)piperidine-2,6-dione (74.47 mg, 0.22 mmol), tris(trimethylsilyl)silane (54.7 mg, 0.22 mmol), Na₂CO₃ (70.1 mg, 0.66 mmol) and IR[DF(CF₃)PPY]₂(DTBPY)PF₆ (1.00 mg, 0.002 mmol) in DME (4.00 mL) was added a mixture of dtbbpy (0.59 mg, 0.002 mmol) and 1,2-dimethoxyethane dihydrochloride nickel (0.48 mg, 0.002 mmol) in DME (2.00 mL) at room temperature under nitrogen atmosphere. The resulting mixture was stirred for 4 h under 34 W blue LEDs at room temperature. The resulting mixture was filtered. The filter cake was washed with DCM (2×10.0 mL). The combined filtrate was concentrated under reduced pressure. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 55%-75% B in 25 min; Flow rate: 80 mL/min; Detector: UV 220/254 nm; desired fractions were collected at 58% B and concentrated under reduced pressure to afford tert-butyl 4-[trans-3-[2-[1-(2,6-dioxopiperidin-3-yl)-3-methyl-2-oxo-1,3-benzodiazol-5-yl]ethoxy]cyclobutyl]piperazine-1-carboxylate (50.0 mg, 42%) as white solid. ¹H NMR (400 MHz, DMSO-d₆) δ 7.08 (s, 1H), 7.01 (d, J=2.2 Hz, 2H), 5.31 (m, 1H), 4.05 (m, 1H), 3.59 (t, J=6.7 Hz, 2H), 3.48-3.43 (m, 2H), 3.42 (s, 4H), 2.90 (q, J=7.3, 6.9 Hz, 3H), 2.85-2.81 (m, 1H), 2.81-2.72 (m, 1H), 2.39 (s, 1H), 2.18-2.14 (m, 3H), 2.08 (d, J=7.5 Hz, 1H), 2.03 (s, 2H), 1.45 (s, 9H). LC/MS (ESI, m/z): [(M+1)]⁺=542.30.

The following intermediates in Table 20 were synthesized according to the above procedure:

TABLE 20

| Inter- | | | | |
|---|---|---|---|---|
| medi- | | | MS: | |
| ate | Structure | Name | [(M +1)]⁺ | ¹H NMR |
| R3 | 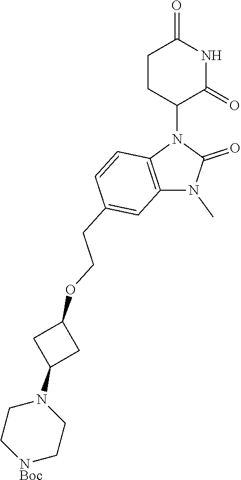 | tert-butyl 4-[cis-3- [2-[1-(2,6- dioxopiperidin-3- yl)-3-methyl-2-oxo- 1,3-benzodiazol-5- yl]ethoxy]cyclobutyl] piperazine-1- carboxylate | 542.25 | (400 MHz, DMSO-d₆) δ 11.08 (s, 1H), 7.07 (s, 1H), 7.01 (d, J = 8.0 Hz, 1H), 6.90 (d, J = 7.9 Hz, 1H), 5.34 (m, 1H), 3.67 (m, 2H), 3.50 (t, J = 7.1 Hz, 2H), 2.92 (d, J = 13.6 Hz, 1H), 2.72-2.59 (m, 2H), 2.36 (d, J = 9.4 Hz, 3H), 2.30-2.22 (m, 2H), 2.17 (t, J = 5.0 Hz, 6H), 1.62-1.58 (m, 2H), 1.39 (s, 9H), 1.08 (t, J = 7.0 Hz, 2H) |
| R4 | 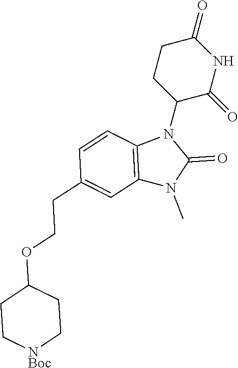 | tert-butyl 4-[2-[1- (2,6- dioxopiperidin-3- yl)-3-methyl-2-oxo- 1,3-benzodiazol-5- yl]ethoxy]piperidine- 1-carboxylate | 487.25 | (400 MHz, DMSO-d₆) δ 7.10 (d, J = 1.6 Hz, 1H), 7.02 (d, J = 8.0 Hz, 1H), 6.92 (m, 1H), 5.34 (m, 1H), 3.63 (t, J = 7.2 Hz, 3H), 3.60-3.56 (m, 1H), 3.48 (m, 1H), 3.02 (s, 1H), 2.97-2.80 (m, 3H), 2.78-2.57 (m, 2H), 2.08 (s, 2H), 2.05-1.97 (m, 1H), 1.77 (d, J = 12.8 Hz, 2H), 1.39 (s, 9H), 1.32 (m, 2H) |
| R5 | 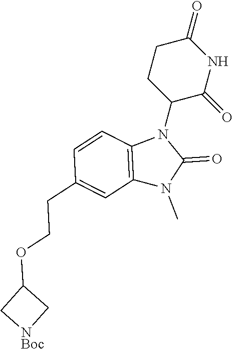 | tert-butyl 3-[2-[1- (2,6- dioxopiperidin-3- yl)-3-methyl-2-oxo- 2,3-dihydro-₁H-1,3- benzodiazol-5- yl]ethoxy]azetidine- 1-carboxylate | 459.10 | (400 MHz, CDCl₃) δ 8.21 (s, 1H), 6.98-6.89 (m, 2H), 6.75 (d, J = 8.0 Hz, 1H), 5.23 (m, 1H), 4.22 (m, 1H), 4.07 (m, 2H), 3.83 (m, 2H), 3.59 (t, J = 6.9 Hz, 2H), 3.45 (s, 3H), 2.95 (q, J = 7.7, 6.7 Hz, 3H), 2.79 (m, 2H), 2.29-2.21 (m, 1H), 1.45 (s, 9H) |
| R6 | 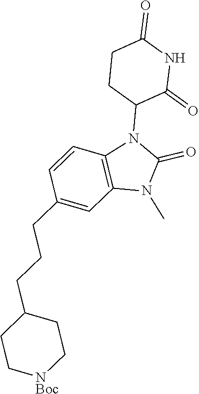 | tert-butyl 4-[3-[1- (2,6- dioxopiperidin-3- yl)-3-methyl-2-oxo- 1,3-benzodiazol-5- yl]propyl]piperidine- 1-carboxylate | 485.25 | (400 MHz, CDCl₃) δ 8.16 (s, 1H), 6.90 (m, 1H), 6.86 (d, J = 1.5 Hz, 1H), 6.73 (d, J = 8.0 Hz, 1H), 5.23 (m, 1H), 4.09 (m, 2H), 3.45 (s, 3H), 3.01-2.71 (m, 4H), 2.71-2.62 (m, 4H), 2.30-2.21 (m, 1H), 2.03 (s, 1H), 1.70-1.62 (m, 4H), 1.47 (s, 9H), 1.31 (m, 2H), 1.10 (m, 2H) |

### INTERMEDIATE S1: 1-(2,6-dioxopiperidin-3-yl)-3-methyl-2-oxo-1,3-benzodiazole-5-carbaldehyde

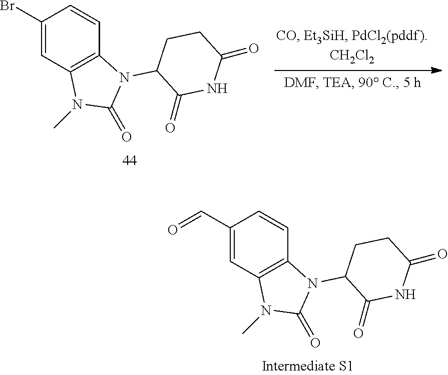 [SMILES: Cn1c(=O)n(C2CCC(=O)NC2=O)c2ccc(Br)cc21 ; Cn1c(=O)n(C2CCC(=O)NC2=O)c2ccc(C=O)cc21]

To a stirred solution of 3-(5-bromo-3-methyl-2-oxo-2,3-dihydro-1H-1,3-benzodiazol-1-yl)piperidine-2,6-dione (200 mg, 0.59 mmol), Et₃SiH (136 mg, 1.18 mmol) and TEA (180 mg, 1.77 mmol) in DMF (10.0 mL) was added Pd(dppf)Cl₂ (50.0 mg, 68.3 mmol) at room temperature under nitrogen atmosphere. The mixture was allowed to react for 16 h at 110 degrees C. under CO atmosphere. After completion, the mixture was cooled down to room temperature and concentrated by reversed phase flash chromatography with the following conditions: Column: WelFlash™ C18-I, 20-40 um, 80 g; Eluent A: Water (plus 10 mmol/L FA); Eluent B: ACN; Gradient: 15%-30% B in 15 min; Flow rate: 50 mL/min; Detector: 220/254 nm; desired fractions were collected at 25% B and concentrated under reduced pressure to afford 1-(2,6-dioxopiperidin-3-yl)-3-methyl-2-oxo-2,3-dihydro-1H-1,3-benzodiazole-5-carbaldehyde (110 mg, 65%) as an off white solid. ¹H NMR (400 MHz, DMSO-d₆) δ 11.16 (s, 1H), 9.95 (s, 1H), 7.73-7.70 (m, 2H), 7.37 (d, J=8.4 Hz, 1H), 5.51-5.48 (m, 1H), 3.43 (s, 3H), 2.98-2.90 (m, 1H), 2.80-2.75 (m, 1H), 2.71-2.60 (m, 1H), 2.13-2.03 (m, 1H). LC/MS (ESI, m/z): [(M+1)]⁺=288.15

### INTERMEDIATE T1: 3-[5-(hydroxymethyl)-3-methyl-2-oxo-1,3-benzodiazol-1-yl]piperidine-2,6-dione

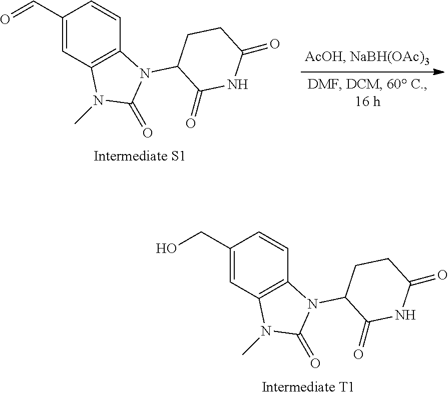 [SMILES: Cn1c(=O)n(C2CCC(=O)NC2=O)c2ccc(C=O)cc21 ; Cn1c(=O)n(C2CCC(=O)NC2=O)c2ccc(CO)cc21]

To a stirred solution of 1-(2,6-dioxopiperidin-3-yl)-3-methyl-2-oxo-1,3-benzodiazole-5-carbaldehyde (5.00 g, 17.41 mmol) and AcOH (4.18 g, 69.6 mmol) in DCM (40.0 mL) and DMF (40.0 mL) was added NaBH(OAc)₃ (36.9 g, 174 mmol) at room temperature. The reaction was stirred at 60° C. for 16 h. The mixture was cooled down to room temperature and concentrated under reduced pressure. The residue was purified by reversed phase flash chromatography with the following conditions: Column: WelFlash™ C18-I, 20-40 um, 330 g; Eluent A: Water (plus 10 mmol/L FA); Eluent B: ACN; Gradient: 15%-30% B in 15 min; Flow rate: 50 mL/min; Detector: UV 220/254 nm; desired fractions were collected at 20% B and concentrated under reduced pressure to afford 3-[5-(hydroxymethyl)-3-methyl-2-oxo-1,3-benzodiazol-1-yl]piperidine-2,6-dione (3.00 g, 60%) as a white solid. ¹H NMR (400 MHz, DMSO-d₆) δ 11.09 (s, 1H), 7.14 (d, J=1.5 Hz, 1H), 7.06 (d, J=8.0 Hz, 1H), 7.00 (dd, J=8.1, 1.5 Hz, 1H), 5.40-5.30 (m, 1H), 5.15 (t, J=5.7 Hz, 1H), 4.52 (d, J=5.6 Hz, 2H), 3.34 (s, 3H), 2.94-2.90 (m, 1H), 2.79-2.55 (m, 2H), 2.10-1.96 (m, 1H). LC/MS (ESI, m/z): [(M+1)]⁺=290.20

### INTERMEDIATE R7: tert-butyl 4-[[1-(2,6-dioxopiperidin-3-yl)-3-methyl-2-oxo-1,3-benzodiazol-5-yl]methoxy]piperidine-1-carboxylate

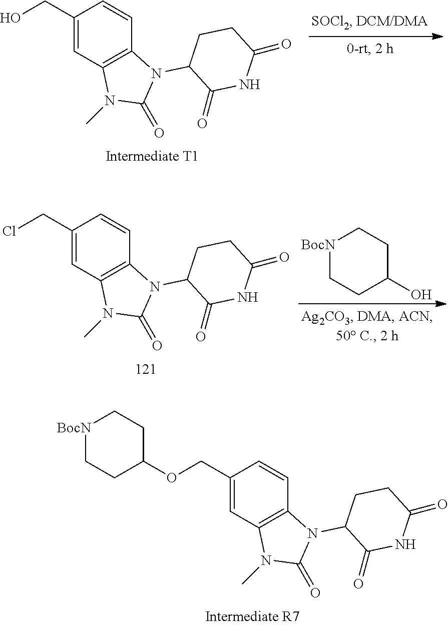 [SMILES: Cn1c(=O)n(C2CCC(=O)NC2=O)c2ccc(CO)cc21 ; Cn1c(=O)n(C2CCC(=O)NC2=O)c2ccc(CCl)cc21 ; CC(C)(C)OC(=O)N1CCC(O)CC1 ; Cn1c(=O)n(C2CCC(=O)NC2=O)c2ccc(COC3CCN(C(=O)OC(C)(C)C)CC3)cc21]

### Step 1: 3-[5-(chloromethyl)-3-methyl-2-oxo-1,3-benzodiazol-1-yl]piperidine-2,6-dione

To a stirred solution of 3-[5-(hydroxymethyl)-3-methyl-2-oxo-1,3-benzodiazol-1-yl]piperidine-2,6-dione (500 mg, 1.73 mmol) in DCM (20.0 mL) and DMA (10.0 mL) was added SOCl₂ (411 mg, 3.46 mmol) dropwise at 0° C. under nitrogen atmosphere. The resulting solution was stirred for 2 h at room temperature. The resulting solution was diluted with water (40.0 mL) and extracted with CH₂Cl₂ (3×30.0 mL). The combined organic layers were washed with water (2×30.0 mL) and dried over anhydrous Na₂SO₄. After filtration, the filtrate was concentrated under reduced pressure to afford 3-[5-(chloromethyl)-3-methyl-2-oxo-1,3-benzodiazol-1-yl]piperidine-2,6-dione (500 mg, 94%) as a yellow solid. ¹H NMR (400 MHz, DMSO-d₆) δ 11.11 (s, 1H), 7.29 (d, J=1.4 Hz, 1H), 7.17-7.09 (m, 2H), 5.42-5.38 (m, 1H), 4.83 (s, 2H), 3.36 (s, 3H), 2.93-2.85 (m, 1H), 2.75-2.70 (m, 1H), 2.69-2.59 (m, 1H), 2.05-2.00 (m, 1H). LC/MS (ESI, m/z): [(M+1)]⁺=308.10, 310.10

### Step 2: tert-butyl 4-[[1-(2,6-dioxopiperidin-3-yl)-3-methyl-2-oxo-1,3-benzodiazol-5-yl]methoxy]piperidine-1-carboxylate

To a stirred mixture of tert-butyl 4-hydroxypiperidine-1-carboxylate (981 mg, 4.87 mmol) and Ag₂CO₃ (1344 mg, 4.87 mmol) in ACN (20.0 mL)/DMA (5.00 mL) was added 3-[5-(chloromethyl)-3-methyl-2-oxo-1,3-benzodiazol-1-yl]piperidine-2,6-dione (500 mg, 1.63 mmol) at room temperature under nitrogen atmosphere. The mixture was stirred for 2 h at 50° C. Upon completion, the resulting mixture was cooled and filtered. The filter cake was washed with CH₂Cl₂ (3×10.0 mL). The filtrate was concentrated under reduced pressure. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 35%-55% B in 20 min; Flow rate: 80 mL/min; Detector: UV 220/254 nm; desired fractions were collected at 46% B and concentrated under reduced pressure to afford tert-butyl 4-[[1-(2,6-dioxopiperidin-3-yl)-3-methyl-2-oxo-1,3-benzodiazol-5-yl]methoxy]piperidine-1-carboxylate (350 mg, 46%) as a white solid. ¹H NMR (400 MHz, DMSO-d₆) δ 11.09 (s, 1H), 7.16 (d, J=1.4 Hz, 1H), 7.08 (d, J=8.0 Hz, 1H), 7.04-7.00 (m, 1H), 5.40-5.35 (m, 1H), 4.53 (s, 2H), 3.66-3.62 (m, 2H), 3.59-3.34 (m, 1H), 3.35 (s, 3H), 3.05 (d, J=11.1 Hz, 2H), 2.94-2.84 (m, 1H), 2.75-2.70 (m, 1H), 2.67-2.59 (m, 1H), 2.07-1.98 (m, 1H), 1.85-1.80 (m, 2H), 1.40 (s, 11H). LC/MS (ESI, m/z): [(M+1)]⁺=372.15

### INTERMEDIATE U1: tert-butyl 3-(prop-2-yn-1-yl)azetidine-1-carboxylate

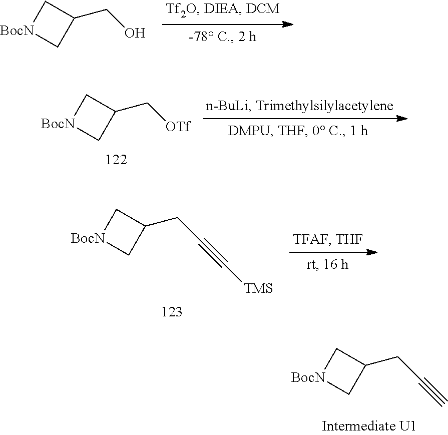 [SMILES: CC(C)(C)OC(=O)N1CC(CO)C1 ; CC(C)(C)OC(=O)N1CC(COS(=O)(=O)C(F)(F)F)C1 ; CC(C)(C)OC(=O)N1CC(CC#C[Si](C)(C)C)C1 ; C#CCC1CN(C(=O)OC(C)(C)C)C1]

### Step 1: tert-butyl 3-[(trifluoromethanesulfonyloxy)methyl]azetidine-1-carboxylate

To a stirred solution of tert-butyl 3-(hydroxymethyl)azetidine-1-carboxylate (10.0 g, 53.4 mmol) in DCM (100 mL) was added DIEA (17.3 g, 134 mmol) dropwise at room temperature under nitrogen atmosphere. To the above mixture was added Tf₂O (18.1 g, 64.1 mmol) dropwise at −78° C. The resulting mixture was stirred for 2 h at −78° C. The resulting mixture was quenched with water (100 mL) and extracted with EtOAc (3×200 mL). The combined organic layers were washed with NaHCO₃ (200 mL), brine (200 mL) and dried over anhydrous Na₂SO₄. After filtration, the filtrate was concentrated under reduced pressure. The residue was purified by silica gel column chromatography, eluted with Petroleum ether/EtOAc (v:v=20:1) to afford tert-butyl 3-[(trifluoromethanesulfonyloxy)methyl]azetidine-1-carboxylate (9.20 g, 54%) as a yellow oil. ¹H NMR (400 MHz, DMSO-d₆) δ 4.42 (d, J=6.7 Hz, 2H), 3.97-3.86 (m, 2H), 3.68-3.55 (m, 2H), 2.96-2.86 (m, 1H), 1.38 (s, 9H)

### Step 2: tert-butyl 3-[3-(trimethylsilyl)prop-2-yn-1-yl]azetidine-1-carboxylate

To a stirred solution of ethynyltrimethylsilane (8.83 g, 89.9 mmol) in THF (150 mL) was added n-BuLi (2.5 M in THF, 43.0 mL, 108 mmol) dropwise at 0° C. under nitrogen atmosphere. The resulting mixture was stirred for 30 min at 0° C. under nitrogen atmosphere. DMPU (25.8 mL) was then added at −20° C., followed by the addition of a solution of tert-butyl 3-[(trifluoromethanesulfonyloxy)methyl]azetidine-1-carboxylate (5.00 g, 15.7 mmol) in THF (20.0 mL) and the reaction was stirred for 30 min at −20° C. Conc. ammonia (8.40 mL) and MeOH (8.40 mL) were then added at −20° C. and the reaction was warmed to room temperature over 10 min. The mixture was diluted with EtOAc (150 mL) and water (100 mL). The organic layer was washed with water (2×50.0 mL), brine (100 mL), dried over anhydrous Na₂SO₄ and filtered. The filtrate was concentrated under vacuum to afford tert-butyl 3-[3-(trimethylsilyl)prop-2-yn-1-yl]azetidine-1-carboxylate (2.20 g, 53%) as a yellow oil. ¹H NMR (400 MHz, CDCl₃) δ 3.99 (t, J=8.4 Hz, 2H), 3.68 (m, 2H), 2.73-2.61 (m, 1H), 2.46 (d, J=6.8 Hz, 2H), 1.44 (s, 9H), 0.14 (s, 9H)

### Step 3: tert-butyl 3-(prop-2-yn-1-yl)azetidine-1-carboxylate

To a stirred solution of tert-butyl 3-[3-(trimethylsilyl)prop-2-yn-1-yl]azetidine-1-carboxylate (1.60 g, 5.98 mmol) in THF (20.0 mL) was added TBAF (3.13 g, 12.0 mmol) at room temperature under nitrogen atmosphere. The resulting solution was stirred for 16 h at room temperature. The resulting mixture was concentrated under vacuum. The residue was purified by silica gel column chromatography, eluted with Petroleum ether/EtOAc (v:v=10:1) to afford tert-butyl 3-(prop-2-yn-1-yl)azetidine-1-carboxylate (1.10 g, 94%) as a colorless oil. ¹H NMR (400 MHz, CDCl₃) δ 4.00 (t, J=8.4 Hz, 2H), 3.67 (m, 2H), 2.75-2.63 (m, 1H), 2.42 (m, 2H), 1.97 (t, J=2.6 Hz, 1H), 1.42 (s, 9H)

### INTERMEDIATE U2: tert-butyl 4-[3-(prop-2-yn-1-yloxy)propoxy]piperidine-1-carboxylate

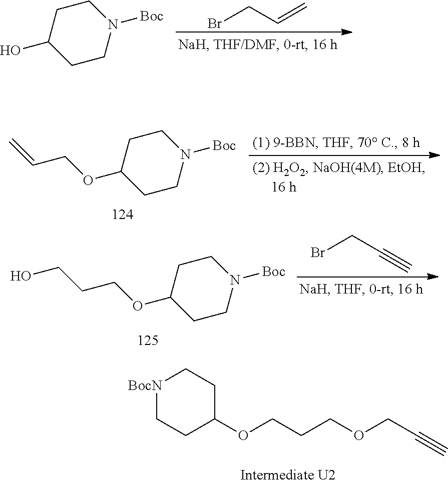

### Step 1: tert-butyl 4-(prop-2-en-1-yloxy)piperidine-1-carboxylate

To a stirred solution of tert-butyl 4-hydroxypiperidine-1-carboxylate (10.0 g, 49.7 mmol) in THF (100 mL) was added NaH (3.58 g, 149 mmol, 60% dispersion in mineral oil) at 0° C. To the above mixture was added allyl bromide (12.1 g, 99.4 mmol) dropwise over 1 h at 0° C. The resulting mixture was stirred for additional 16 h at room temperature. The reaction was quenched with water (500 mL) at room temperature. The resulting mixture was extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with Petroleum ether/EtOAc (v:v=10:1) to afford tert-butyl 4-(prop-2-en-1-yloxy)piperidine-1-carboxylate (10.0 g, 83%) as a yellow oil. ¹H NMR (400 MHz, DMSO-d₆) δ 5.90 (m, 1H), 5.26 (m, 1H), 5.13 (m, 1H), 3.99 (m, 2H), 3.63 (m, 2H), 3.49 (m, 1H), 3.02 (t, J=11.3 Hz, 2H), 1.79 (m, 2H), 1.40 (s, 9H), 1.33 (m, 2H).

The following intermediates in Table 21 were synthesized according to the above procedure:

TABLE 21

| Inter- | | | | |
|---|---|---|---|---|
| medi- | | | MS: | |
| ate | Structure | Name | [(M + 1)]⁺ | ¹H NMR |
| 124-1 | 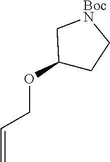 | tert-butyl (3R)- 3-(prop-2-en-1- yloxy) pyrrolidine-1- carboxylate | [(2M + 1)]⁺ = 455.35 | (400 MHz, DMSO-d₆) δ 5.91-5.87 (m, 1H), 5.30-5.20 (m, 1H), 5.16-5.12 (m, 1H), 4.06 (s, 1H), 4.00-3.94 (m, 2H), 3.26 (d, J = 13.5 Hz, 4H), 1.91 (s, 2H), 1.41 (s, 9H) |
| 124-2 | 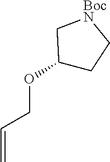 | tert-butyl (3S)- 3-(prop-2-en-1- yloxy) pyrrolidine-1- carboxylate | [(2M + 1)]⁺ = 455.35 | (400 MHz, DMSO-d₆) δ 5.92-5.85 (m, 1H), 5.30-5.20 (m, 1H), 5.18-5.10 (m, 1H), 4.10-4.00 (m, 1H), 3.98-3.92 (m, 2H), 3.30-3.16 (m, 4H), 1.94-1.88 (m, 2H), 1.40 (s, 9H) |
| 124-3 | 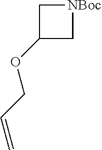 | tert-butyl 3- hydroxyazetidine- 1-carboxylate | [(2M + 1)]⁺ = 427.35 | (400 MHz, DMSO-d₆) δ 5.91-5.86 (m, 1H), 5.30-5.25 (m, 1H), 5.20-5.15 (m, 1H), 4.29-4.22 (m, 1H), 4.00 (t, J = 7.8 Hz, 2H), 3.95-3.88 (m, 2H), 3.73-3.60 (m, 2H), 1.38 (s, 9H) |

### Step 2: tert-butyl 4-(3-hydroxypropoxy)piperidine-1-carboxylate

To a stirred solution of tert-butyl 4-(prop-2-en-1-yloxy)piperidine-1-carboxylate (10.0 g, 41.4 mmol) in THF (40.0 mL) was added 9-borabicyclo[3.3.1]nonane (62.0 mL, 62.2 mmol, 1 M in THF) at 0° C. under nitrogen atmosphere. The reaction was stirred at 70° C. for 8 h. The mixture was cooled down to room temperature. To the resulting solution were added aq. NaOH (50.0 mL, 4_{M}), H₂O₂ (30%, 50.0 mL) and EtOH (50.0 mL) at room temperature and stirred for 16 h. The resulting mixture was extracted with EtOAc (2×500 mL). The combined organic layers were washed with brine (500 mL) and dried over anhydrous Na₂SO₄. After filtration, the filtrate was concentrated under reduced pressure. The residue was purified by silica gel column chromatography, eluted with Petroleum ether/EtOAc (v:v=1:1) to afford tert-butyl 4-(3-hydroxypropoxy)piperidine-1-carboxylate (9.00 g, 84%) as a yellow oil. ¹H NMR (400 MHz, DMSO-d₆) δ 4.36 (t, J=5.1 Hz, 1H), 3.65-3.58 (m, 2H), 3.50-3.38 (m, 5H), 3.03 (d, J=12.2 Hz, 2H), 1.81-1.70 (m, 2H), 1.65-1.60 (m, 2H), 1.39 (s, 9H), 1.34-1.28 (m, 2H).

The following intermediates in Table 22 were synthesized according to the above procedure:

TABLE 22

| Inter- | | | MS: | |
|---|---|---|---|---|
| medi- | | | [(M + | |
| ate | Structure | Name | 1)]⁺ | ¹H NMR |
| 125-1 | 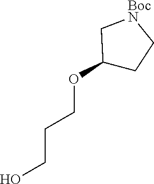 [SMILES: CC(C)(C)OC(=O)N1CC[C@@H](OCCCO)C1] | tert-butyl (3R)-3- (3- hydroxypropoxy) pyrrolidine-1- carboxylate | 246.10 | (400 MHz, DMSO-d₆) 4.38 (t, J = 5.2 Hz, 1H), 3.99 (s, 1H), 3.48-3.39 (m, 4H), 3.28 (s, 2H), 3.25-3.15 (m, 2H), 1.88 (s, 2H), 1.65-1.60 (m), 1.40 (s, 9H) |
| 125-2 | 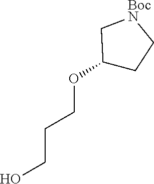 [SMILES: CC(C)(C)OC(=O)N1CC[C@H](OCCCO)C1] | tert-butyl (3S)-3- (3- hydroxypropoxy) pyrrolidine-1- carboxylate | 246.10 | (400 MHz, DMSO-d₆) δ 4.38 (t, J = 5.1 Hz, 1H), 3.44 (t, J = 6.4 Hz, 4H), 3.30 (d, J = 18.1 Hz, 3H), 3.21 (d, J = 10.7 Hz, 2H), 1.90-1.85 (m, 2H), 1.62 (p, J = 6.4 Hz, 2H), 1.40 (s, 9H) |
| 125-3 | 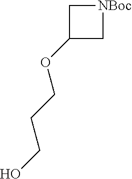 [SMILES: CC(C)(C)OC(=O)N1CC(OCCCO)C1] | tert-butyl 3-(3- hydroxypropoxy) azetidine-1- carboxylate | 463.30 [(2M + 1)]⁺ | (400 MHz, DMSO-d₆) δ 4.42 (t, J = 5.2 Hz, 1H), 4.19 (m, 1H), 4.00 (t, J = 8.0 Hz, 2H), 3.63 (m, 2H), 3.45 (m, 2H), 3.38 (t, J = 6.5 Hz, 2H), 1.64 (m, 2H), 1.38 (s, 9H) |

### Step 3: tert-butyl 4-[3-(prop-2-yn-1-yloxy)propoxy]piperidine-1-carboxylate

To a solution of tert-butyl 4-(3-hydroxypropoxy)piperidine-1-carboxylate (11.3 g, 43.6 mmol) in THF (120 mL) was added NaH (2.61 g, 65.4 mmol, 60% dispersion in mineral oil) at 0° C. The mixture was stirred at 0° C. for 30 min then a solution of 3-bromoprop-1-yne (7.77 g, 65.4 mmol) in THF (80.0 mL) was added at 0° C. The mixture was stirred at 25° C. for 16 h. The reaction was quenched by the addition of sat. NH₄Cl (aq.) (100 mL) at 0° C. and was extracted with EtOAc (3×200 mL). The combined organic layers were washed with brine (200 mL) and dried over anhydrous Na₂SO₄. After filtration, the filtrate was concentrated under reduced pressure. The residue was purified by silica gel column chromatography, eluted with Petroleum ether/EtOAc (v:v=20:1 to 5:1) to afford tert-butyl 4-[3-(prop-2-yn-1-yloxy)propoxy]piperidine-1-carboxylate (9.10 g, 70%) as a yellow oil. ¹H NMR (400 MHz, CDCl₃) δ 4.15 (d, J=2.4 Hz, 2H), 3.80-3.70 (m, 2H), 3.64-3.57 (m, 4H), 3.49-3.44 (m, 1H), 3.15-3.08 (m, 2H), 2.51-2.35 (m, 1H), 1.96-1.75 (m, 4H), 1.47 (s, 11H). LC/MS (ESI, m/z): [(M+1)]⁺=398.20.

The following intermediates in Table 23 were synthesized according to the above procedure:

TABLE 23

| Inter- | | | | |
|---|---|---|---|---|
| medi- | | | MS: | |
| ate | Structure | Name | [(M + 1)]⁺ | ¹H NMR |
| U3 | 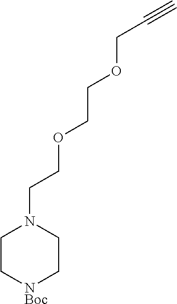 [SMILES: C#CCOCCOCCN1CCN(C(=O)OC(C)(C)C)CC1] | tert-butyl 4-[2-[2- (prop-2-yn-1- yloxy)ethoxy] ethyl]piperazine-1- carboxylate | 313.10 | (400 MHz, DMSO-d₆) δ 4.15 (d, J = 2.4 Hz, 2H), 3.60-3.40 (m, 6H), 3.35 (s, 1H), 3.35-3.30 (m, 2H), 2.58-2.37 (m, 4H), 2.35 (d, J = 5.1 Hz, 4H), 1.40 (s, 9H) |

### INTERMEDIATE U4: tert-butyl 4-[2-(prop-2-yn-1-yloxy)ethoxy]piperidine-1-carboxylate

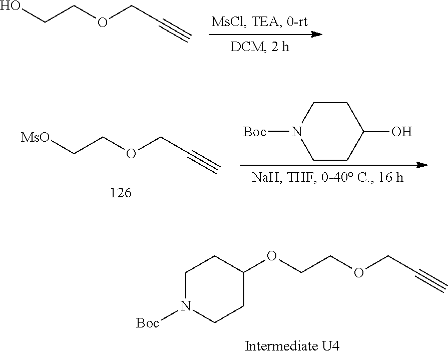

### Step 1: 2-(prop-2-yn-1-yloxy)ethyl methanesulfonate

To a stirred solution of 2-(prop-2-yn-1-yloxy)ethanol (7.90 g, 78.9 mmol) in DCM (80.0 mL) was added TEA (15.9 g, 158 mmol) followed by the addition of a solution of methanesulfonyl chloride (13.6 g, 119 mmol) at 0° C. under nitrogen atmosphere. The resulting solution was stirred for additional 2 h at room temperature. Upon completion, the reaction was quenched with water (100 mL) at 0° C. The mixture was extracted with EtOAc (2×100 mL). The combined organic phase was washed with brine (100 mL), dried with anhydrous Na₂SO₄ and filtered. The filtrate was concentrated under reduced pressure to afford 2-(prop-2-yn-1-yloxy)ethyl methanesulfonate (14.0 g, 99%) as a colorless oil. ¹H NMR (400 MHz, DMSO-d₆) δ 4.37-4.28 (m, 2H), 4.21 (d, J=2.4 Hz, 2H), 3.76-3.63 (m, 2H), 3.53-3.42 (m, 1H), 3.19 (s, 3H).

The following intermediates in Table 24 were synthesized according to the above procedure:

TABLE 24

| Inter- | | | |
|---|---|---|---|
| mediate | Structure | Name | ¹H NMR |
| 127 | 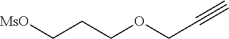 | 3-(prop-2-yn-1- yloxy)propyl methanesulfonate | (400 MHz, DMSO-d) 4.24 (t, J = 6.3 Hz, 2H), 4.14 (d, J = 2.4 Hz, 2H), 3.58-3.50 (m, 2H), 3.32 (s, 1H), 2.75 (s, 3H), 1.92 (d, J = 6.3 Hz, 2H) |

### Step 2: tert-butyl 4-[2-(prop-2-yn-1-yloxy)ethoxy]piperidine-1-carboxylate

To a stirred solution of tert-butyl 4-hydroxypiperidine-1-carboxylate (59.3 g, 295 mmol) in THF (1.00 L) was added NaH (19.6 g, 491 mmol, 60% dispersion in mineral oil) at 0° C. under nitrogen atmosphere. After stirring for 1 h at room temperature, a solution of 2-(prop-2-yn-1-yloxy)ethyl methanesulfonate (35.0 g, 196 mmol) in THF (50.0 mL) was added dropwise. The resulting mixture was stirred for additional 16 at 40° C. The reaction was quenched with water (300 mL) at 0° C. The mixture was extracted with EtOAc (3×200 mL). The combined organic phase was washed with brine (300 mL), dried with anhydrous Na₂SO₄ and filtered. The filtrate was concentrated under reduced pressure. The residue was purified by silica gel column chromatography, eluted with Petroleum ether/EtOAc (v:v=20:1 to 5:1) to give tert-butyl 4-[2-(prop-2-yn-1-yloxy)ethoxy]piperidine-1-carboxylate (23.0 g, 41%) as a yellow oil. ¹HNMR (400 MHz, DMSO-d₆) δ 4.18-4.12 (m, 2H), 3.66-3.60 (m, 4H), 3.52-3.39 (m, 2H), 3.12-2.94 (m, 2H), 1.83-1.73 (m, 2H), 1.47-1.22 (m, 13H). LC/MS (ESI, m/z): [(M+1)]⁺=285.15.

The following intermediates in Table 25 were synthesized according to the above procedure:

TABLE 25

| Inter- | | | | |
|---|---|---|---|---|
| medi- | | | MS: | |
| ate | Structure | Name | [(M +1)]⁺ | ¹H NMR |
| U5 | 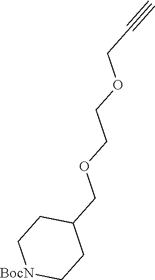 [SMILES: C#CCOCCOCC1CCN(C(=O)OC(C)(C)C)CC1] | tert-butyl 4-[[2- (prop-2-yn-1- yloxy)ethoxy] methyl]piperidine- 1-carboxylate | 298.40 | (400 MHz, DMSO-d₆) δ 4.15 (d, J = 2.4 Hz, 2H), 3.93 (d, J = 13.0 Hz, 2H), 3.59- 3.47 (m, 4H), 3.43 (t, J = 2.4 Hz, 1H), 3.24 (d, J = 6.3 Hz, 2H), 2.68 (s, 2H), 1.75-1.66 (m, 1H), 1.66-1.58 (m, 2H), 1.39 (s, 9H), 1.03-0.07 (m, 2H) |
| U6 | 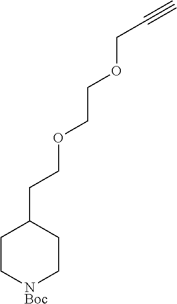 [SMILES: C#CCOCCOCCC1CCN(C(=O)OC(C)(C)C)CC1] | tert-butyl 4-[2- [2-(prop-2-yn- 1- yloxy)ethoxy] ethyl]piperidine- 1-carboxylate | None | (400 MHz, DMSO-d₆) δ 4.14 (d, J = 2.4 Hz, 2H), 3.54 (t, J = 5.7 Hz, 2H), 3.36- 3.29 (m, 3H), 2.81 (d, J = 9.8 Hz, 3H), 1.40 (s, 9H) |
| U7 | 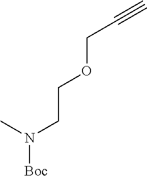 | tert-butyl N- methyl-N-[2- (prop-2-yn-1- yloxy)ethyl] carbamate | None | (400 MHz, DMSO-d₆) δ 4.15 (t, J = 2.2 Hz, 2H), 3.94-3.88 (m, 2H), 3.68-3.56 (m, 1H), 3.56-3.34 (m, 6H), 2.70-2.65 (m, 2H), 1.65-1.60 (m, 2H), 1.55-1.40 (m, 5H), 1.39 (s, 9H) |

### INTERMEDIATE U8: tert-butyl N-(3-[methyl[2-(prop-2-yn-1-yloxy)ethyl]carbamoyl]propyl)carbamate

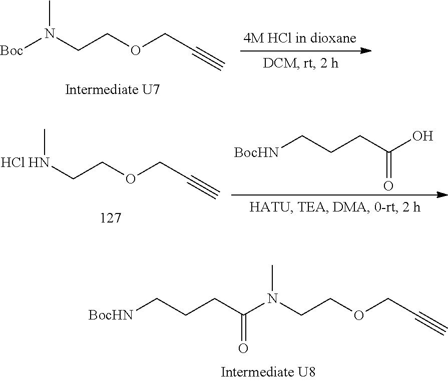

### Step 1: methyl[2-(prop-2-yn-1-yloxy)ethyl]amine hydrochloride

To a stirred solution of tert-butyl N-methyl-N-[2-(prop-2-yn-1-yloxy)ethyl]carbamate (15.0 g, 70.3 mmol) in 1,4-dioxane (100 mL) was added HCl (4 M in 1,4-dioxane, 100 mL) dropwise at 0° C. under nitrogen atmosphere. The resulting mixture was stirred for 2 h at room temperature. The resulting mixture was concentrated under vacuum. This resulted in methyl[2-(prop-2-yn-1-yloxy)ethyl]amine hydrochloride (10.0 g, 95%) as a white solid.

### Step 2: tert-butyl N-(3-[methyl[2-(prop-2-yn-1-yloxy)ethyl]carbamoyl]propyl)carbamate

To a stirred solution of methyl[2-(prop-2-yn-1-yloxy)ethyl]amine hydrochloride (10.0 g, 66.8 mmol), TEA (27.9 mL, 275 mmol) and 4-[[(tert-butoxy)carbonyl]amino]butanoic acid (13.6 g, 66.8 mmol) in DMA (150 mL) was added HATU (30.5 g, 80.2 mmol) at 0° C. under nitrogen atmosphere. The reaction was stirred for 2 h at room temperature. Upon completion, the resulting solution was concentrated under vacuum. The residue was diluted with water (200 mL). The mixture was extracted with EtOAc (3×200 mL). The combined organic phase was washed with brine (500 mL), dried with anhydrous Na₂SO₄ and filtered. The filtrate was concentrated under reduced pressure. The residue was purified by silica gel column chromatography, eluted with Petroleum ether/EtOAc (v:v=5:1 to 1:1) to afford tert-butyl N-(3-[methyl[2-(prop-2-yn-1-yloxy)ethyl]carbamoyl]propyl)carbamate (17.0 g, 95%) as a yellow oil. ¹H NMR (400 MHz, DMSO-d₆) δ 6.81 (d, J=6.6 Hz, 1H), 4.15 (dd, J=11.1, 2.4 Hz, 2H), 3.60-3.50 (m, 2H), 3.14-3.08 (m, 1H), 2.94-2.89 (m, 2H), 2.81 (s, 1H), 2.70 (s, 2H), 2.30-2.25 (m, 2H), 1.61-1.56 (m, 2H), 1.38 (s, 9H), 1.18 (t, J=7.3 Hz, 2H). LC/MS (ESI, m/z): [(M+1)]⁺=299.15

### INTERMEDIATE R8: tert-butyl 3-[3-[1-(2,6-dioxopiperidin-3-yl)-3-methyl-2-oxo-1,3-benzodiazol-5-yl]propyl]azetidine-1-carboxylate

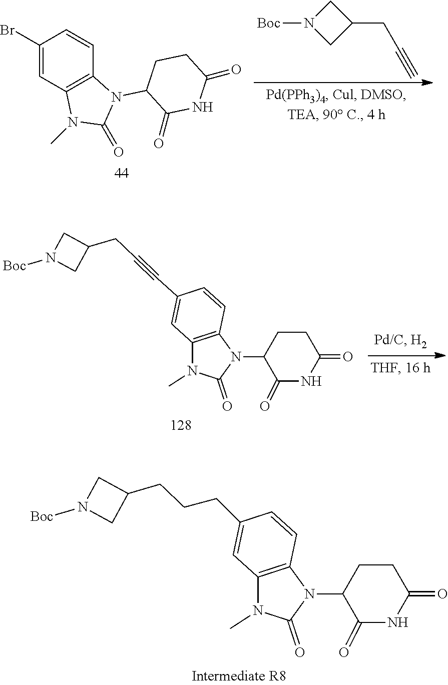 [SMILES: Cn1c(=O)n(C2CCC(=O)NC2=O)c2ccc(Br)cc21 ; C#CCC1CN(C(=O)OC(C)(C)C)C1 ; Cn1c(=O)n(C2CCC(=O)NC2=O)c2ccc(C#CCC3CN(C(=O)OC(C)(C)C)C3)cc21 ; Cn1c(=O)n(C2CCC(=O)NC2=O)c2ccc(CCCC3CN(C(=O)OC(C)(C)C)C3)cc21]

### Step 1: tert-butyl 3-[3-[1-(2,6-dioxopiperidin-3-yl)-3-methyl-2-oxo-1,3-benzodiazol-5-yl]prop-2-yn-1-yl]azetidine-1-carboxylate

To a solution of tert-butyl 3-(prop-2-yn-1-yl)azetidine-1-carboxylate (0.93 g, 4.88 mmol) and 3-(5-bromo-3-methyl-2-oxo-1,3-benzodiazol-1-yl)piperidine-2,6-dione (1.10 g, 3.25 mmol) in TEA (10.0 mL) and DMSO (20.0 mL) were added CuI (730 mg, 0.33 mmol) and Pd(PPh₃)₄ (120 mg, 0.33 mmol) at room temperature. The resulting mixture was stirred for additional 4 h at 90° C. under nitrogen atmosphere. The resulting mixture was cooled and concentrated under vacuum. The residue was purified by reversed phase flash chromatography with the following conditions: Column: Spherical C¹⁸, 20-40 um, 330 g; Mobile Phase A: water (plus 10 mM NH₄HCO₃); Mobile Phase B: ACN; Flow rate: 80 mL/min; Gradient: 5%-5% B, 10 min, 50% B -65% B gradient in 20 min; Detector: UV 220/254 nm. The fractions containing the desired product were collected at 56% B and concentrated under reduced pressure to afford tert-butyl 3-[3-[1-(2,6-dioxopiperidin-3-yl)-3-methyl-2-oxo-1,3-benzodiazol-5-yl]prop-2-yn-1-yl]azetidine-1-carboxylate (300 mg, 20%) as a yellow solid. ¹H NMR (400 MHz, DMSO-d₆) δ 11.08 (s, 1H), 7.06-6.97 (m, 2H), 6.87 (d, J=8.1 Hz, 1H), 5.40-5.30 (m, 1H), 3.89 (s, 1H), 2.96-2.84 (m, 1H), 2.78-2.63 (m, 2H), 2.60 (s,2H), 2.04-1.97 (m, 1H), 1.54 (d, J=3.8 Hz, 4H), 1.36 (s, 9H). LC/MS (ESI, m/z): [(M+1)]⁺=453.15.

The following intermediates in Table 26 were synthesized according to the above procedure:

TABLE 26

| Inter- | | | | |
|---|---|---|---|---|
| medi- | | | MS: | |
| ate | Structure | Name | [(M +1)]⁺ | ¹H NMR |
| 128- 1 | 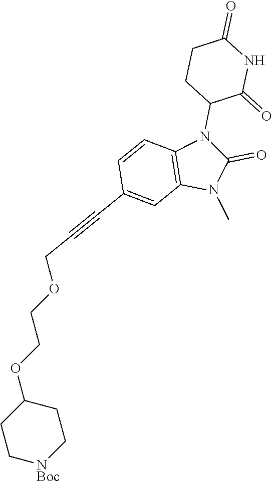 [SMILES: Cn1c(=O)n(C2CCC(=O)NC2=O)c2ccc(C#CCOCCOC3CCN(C(=O)OC(C)(C)C)CC3)cc21] | tert-butyl 4-[2-([3- [1-(2,6- dioxopiperidin-3- yl)-3-methyl-2-oxo- 1,3-benzodiazol-5- yl]prop-2-yn-1- yl]oxy)ethoxy] piperidine-1-carboxylate | 541.30 | (400 MHz, CD₃OD) δ 7.28 (s, 1H), 7.23 (d, J = 8.1 Hz, 1H), 7.10 (d, J = 8.1 Hz, 1H), 4.45 (s, 2H), 3.80-3.68 (m, 5H), 3.60 (m, 1H), 3.44 (s, 3H), 3.16 (d, J = 12.6 Hz, 2H), 2.83 (m, 2H), 2.20 (m, 1H), 2.05 (m, 1H), 1.88 (d, J = 13.4 Hz, 4H), 1.46 (s, 9H) |
| 128- 2 | 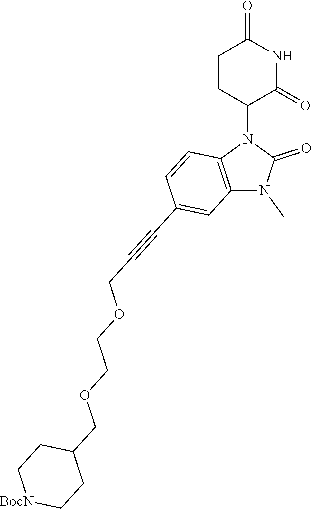 [SMILES: Cn1c(=O)n(C2CCC(=O)NC2=O)c2ccc(C#CCOCCOCC3CCN(C(=O)OC(C)(C)C)CC3)cc21] | tert-butyl 4-[[2-([3- [1-(2,6- dioxopiperidin-3- yl)-3-methyl-2-oxo- 1,3-benzodiazol-5- yl]prop-2-yn-1- yl]oxy)ethoxy]methyl] piperidine-1- carboxylate | 555.30 | (400 MHz, CD₃OD) δ 7.28 (d, J = 1.4 Hz, 1H), 7.23 (dd, J = 8.1, 1.5 Hz, 1H), 7.11 (d, J = 8.2 Hz, 1H), 5.30 (m, 1H), 4.46 (s, 2H), 3.76 (m, 2H), 3.66 (m, 2H), 3.44 (s, 2H), 3.36 (m, 7H), 3.01-2.85 (m, 1H), 2.85-2.73 (m, 4H), 2.25-2.14 (m, 1H), 1.77 (t, J = 15.8 Hz, 4H), 1.46 (d, J = 4.9 Hz, 9H), 1.21 (m, 1H) |
| 128- 3 | 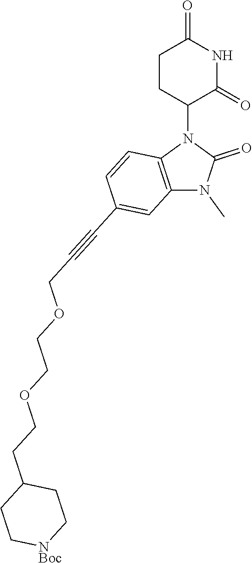 [SMILES: Cn1c(=O)n(C2CCC(=O)NC2=O)c2ccc(C#CCOCCOCCC3CCN(C(=O)OC(C)(C)C)CC3)cc21] | tert-butyl 4-[2-[2- (3-[1-(2,6- dioxopiperidin-3- yl)-3-methyl-2-oxo- 1,3-benzodiazol-5- yl]prop-2-yn-1- yl]oxy)ethoxy]ethyl] piperidine-1- carboxylate | 569.30 | (400 MHz, CD₃OD) δ 7.29 (d, J = 1.5 Hz, 1H), 7.24 (m, 1H), 7.11 (d, J = 8.2 Hz, 1H), 5.36 (m, 1H), 4.44 (s, 2H), 4.04 (d, J = 13.0 Hz, 2H), 3.80-3.73 (m, 2H), 3.66 (m, 2H), 3.58 (t, J = 6.4 Hz, 2H), 3.44 (s, 3H), 3.01-2.89 (m, 1H), 2.89-2.75 (m, 3H), 2.73 (s, 1H), 2.23-2.14 (m, 1H), 1.72 (d, J = 13.6 Hz, 2H), 1.55 (m, 2H), 1.45 (s, 9H), 1.12 (m, 1H) |
| 128- 4 | 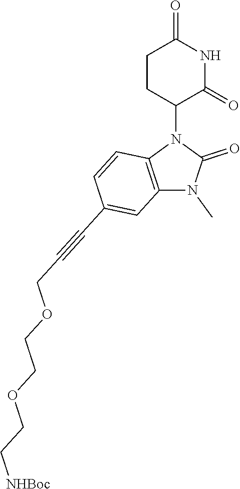 [SMILES: Cn1c(=O)n(C2CCC(=O)NC2=O)c2ccc(C#CCOCCOCCNC(=O)OC(C)(C)C)cc21] | tert-butyl N-[2-[2- (3-[1-(2,6- dioxopiperidin-3- yl)-3-methyl-2-oxo- 2,3-dihydro-1H-1,3- benzodiazol-5- yl]prop-2-yn-1- yl]oxy)ethoxy]ethyl] carbamate | 501.30 | (400 MHz, CDCl₃) δ 8.60 (s, 1H), 7.25-7.20 (m, 1H), 7.13 (d, J = 1.4 Hz, 1H), 6.77 (d, J = 8.2 Hz, 1H), 5.30-5.20 (m, 1H), 5.03 (s, 1H), 4.45 (s, 2H), 3.80-3.74 (m, 2H), 3.72-3.65 (m, 2H), 3.58 (t, J = 5.2 Hz, 2H), 3.43 (s, 3H), 3.40-3.30 (m, 2H), 2.99-2.93 (m, 1H), 2.90-2.65 (m, 2H), 2.28- 2.18 (m, 1H), 1.45 (s, 9H) |
| 128- 5 | 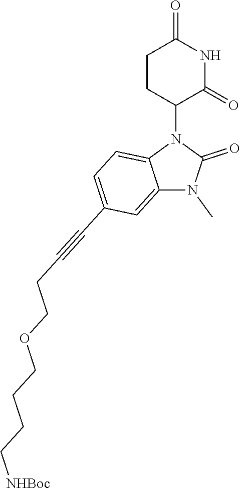 [SMILES: Cn1c(=O)n(C2CCC(=O)NC2=O)c2ccc(C#CCCOCCCCNC(=O)OC(C)(C)C)cc21] | tert-butyl N-[4-([4- [1-(2,6- dioxopiperidin-3- yl)-3-methyl-2-oxo- 2,3-dihydro-1H-1,3- benzodiazol-5- yl]but-3-yn-1- yl]oxy)butyl] carbamate | 499.30 | (400 MHz, DMSO-d₆) δ 11.12 (s, 1H), 7.24 (s, 1H), 7.10 (s, 2H), 6.79 (s, 1H), 5.41-5.34 (m, 1H), 3.55 (t, J = 6.8 Hz, 2H), 3.44 (t, J = 6.3 Hz, 2H), 3.32 (s, 1H), 2.96-2.88 (m, 3H), 2.71 (s, 1H), 2.66 (t, J = 6.7 Hz, 3H), 2.11-2.00 (m, 1H), 1.49 (s, 2H), 1.48-1.40 (m, 2H), 1.37 (s, 9H) |
| 128- 6 | 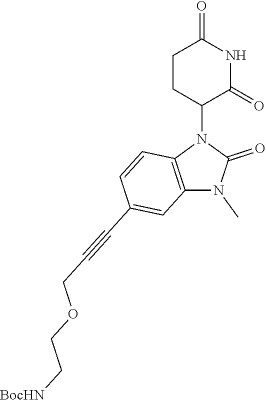 [SMILES: Cn1c(=O)n(C2CCC(=O)NC2=O)c2ccc(C#CCOCCNC(=O)OC(C)(C)C)cc21] | tert-butyl N-[2-([3- [1-(2,6- dioxopiperidin-3- yl)-3-methyl-2-oxo- 2,3-dihydro-1H-1,3- benzodiazol-5- yl]prop-2-yn-1- yl]oxy)ethyl] carbamate | 457.25 | (400 MHz, DMSO-d₆) δ 11.14 (s, 1H), 7.34 (d, J = 1.4 Hz, 1H), 7.21-7.11 (m, 2H), 6.88 (t, J = 5.8 Hz, 1H), 5.45-5.35 (m, 1H), 4.38 (s, 2H), 3.51 (t, J = 6.0 Hz, 2H), 3.32 (s, 3H), 3.13 (q, J = 6.0 Hz, 2H), 2.92-2.84 (m, 1H), 2.75-2.63 (m, 2H), 2.07-2.00 (m, 1H), 1.38 (s, 9H) |
| 128- 7 | 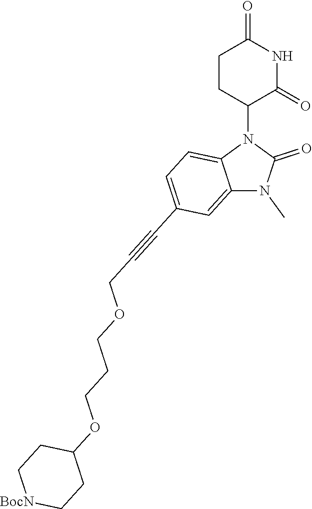 [SMILES: Cn1c(=O)n(C2CCC(=O)NC2=O)c2ccc(C#CCOCCCOC3CCN(C(=O)OC(C)(C)C)CC3)cc21] | tert-butyl 4-[3-([3- [1-(2,6- dioxopiperidin-3- yl)-3-methyl-2-oxo- 2,3-dihydro-1H-1,3- benzodiazol-5- yl]prop-2-yn-1- yl]oxy)propoxy] piperidine-1-carboxylate | 553.30 [(M − 1)]⁻ | (400 MHz, DMSO-d₆) δ 11.13 (s, 1H), 7.32 (s, 1H), 7.21-7.09 (m, 2H), 5.39 (m, 1H), 4.36 (s, 2H), 3.65-3.53 (m, 4H), 3.50- 3.41 (m, 3H), 3.34 (s, 3H), 3.06- 2.84 (m, 3H), 2.77-2.65 (m, 2H), 2.10-2.00 (m, 1H), 1.80-1.70 (m, 4H), 1.37 (s, 9H), 1.34-1.24 (m, 2H) |
| 128- 8 | 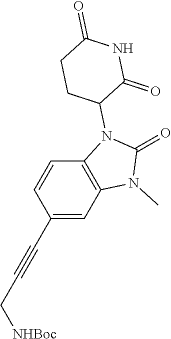 [SMILES: Cn1c(=O)n(C2CCC(=O)NC2=O)c2ccc(C#CCNC(=O)OC(C)(C)C)cc21] | tert-butyl N-[3-[1- (2,6-dioxopiperidin- 3-yl)-3-methyl-2- oxo-2,3-dihydro-1H- 1,3-benzodiazol-5- yl]prop-2-yn-1- yl]carbamate | 357.3 [(M − 56)]⁺ | (400 MHz, CDCl₃) δ 8.27 (s, 1H), 7.20-7.15 (m, 1H), 7.11 (s, 1H), 6.76 (d, J = 8.2 Hz, 1H), 5.25-5.17 (m, 1H), 4.82 (s, 1H), 4.18 (d, J = 5.2 Hz, 2H), 3.44 (s, 3H), 3.01-2.92 (m, 1H), 2.90- 2.85 (m, 1H), 2.80-2.70 (m, 1H), 2.31-2.20 (m, 1H), 1.49 (s, 9H) |
| 128- 9 | 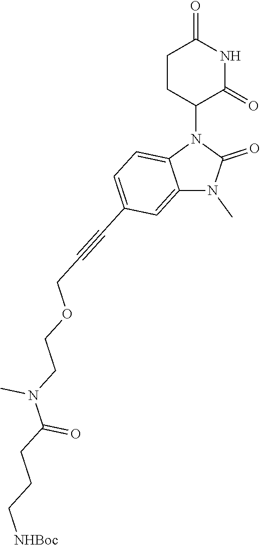 [SMILES: CN(CCOCC#Cc1ccc2c(c1)n(C)c(=O)n2C1CCC(=O)NC1=O)C(=O)CCCNC(=O)OC(C)(C)C] | tert-butyl N-(3-[[2- (3-[1-(2,6- dioxopiperidin-3- yl)-3-methyl-2-oxo- 2,3-dihydro-1H-1,3- benzodiazol-5- yl]prop-2-yn-1- yl]oxy)ethyl](methyl) carbamoyl]propyl) carbamate | 556.40 | (400 MHz, DMSO-d₆) δ 11.13 (s, 1H), 7.73-7.67 (m, 1H), 7.33 (d, J = 5.0 Hz, 1H), 7.15 (s, 1H), 6.80 (s, 1H), 5.44-5.35 (m, 1H), 4.40 (d, J = 10.2 Hz, 2H), 4.22 (t, J = 6.5 Hz, 1H), 3.67-3.59 (m, 1H), 3.54-3.46 (m, 1H), 3.34 (s, 3H), 2.99 (s, 2H), 2.91 (d, J = 6.9 Hz, 2H), 2.83 (s, 1H), 2.37-2.21 (m, 2H), 2.08-1.88 (m, 2H), 1.70-1.60 (m, 2H), 1.59-1.55 (m, 2H), 1.38-1.35 (m, 9H) |
| 128- 10 | 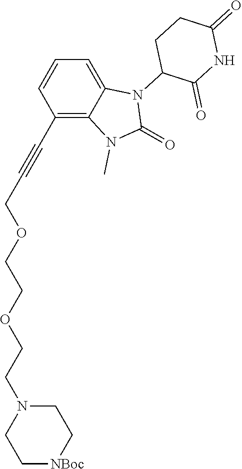 [SMILES: Cn1c(=O)n(C2CCC(=O)NC2=O)c2cccc(C#CCOCCOCCN3CCN(C(=O)OC(C)(C)C)CC3)c21] | tert-butyl 4-[2-[2- ([3-[1-(2,6- dioxopiperidin-3- yl)-3-methyl-2-oxo- 1,3-benzodiazol-4- yl]prop-2-yn-1- yl]oxy)ethoxy]ethyl] piperazine-1- carboxylate | 570.35 | (400 MHz, DMSO-d₆) δ 11.12 (s, 1H), 8.14 (s, 1H), 7.25-7.15 (m 1H), 7.14-7.10 (m, 1H), 5.45- 5.35 (m, 1H), 4.48 (s, 2H), 3.70- 3.60 (m, 2H), 3.65 (s, 4H), 3.62- 3.53 (m, 4H), 3.32 (s, 3H), 2.95- 2.85 (m, 1H), 2.77-2.67 (m, 1H), 2.65 (d, J = 4.7 Hz, 1H), 2.60 (d, J = 6.5 Hz, 2H), 2.50-2.40 (m, 2H), 2.15-2.05 (m, 3H), 1.39 (s, 9H) |

### Step 2: tert-butyl 3-[3-[1-(2,6-dioxopiperidin-3-yl)-3-methyl-2-oxo-1,3-benzodiazol-5-yl]propyl]azetidine-1-carboxylate

To a solution of tert-butyl 3-[3-[1-(2,6-dioxopiperidin-3-yl)-3-methyl-2-oxo-1,3-benzodiazol-5-yl]prop-2-yn-1-yl]azetidine-1-carboxylate (300 mg, 0.66 mmol) in THF (5 mL) was added 10% palladium on activated carbon (50.0 mg) under nitrogen atmosphere. The mixture was stirred for 16 h at room temperature under hydrogen atmosphere using a hydrogen balloon. After filtration, the filtrate was concentrated under reduced pressure to afford tert-butyl 3-[3-[1-(2,6-dioxopiperidin-3-yl)-3-methyl-2-oxo-1,3-benzodiazol-5-yl]propyl]azetidine-1-carboxylate (200 mg, 66%) as a white solid. ¹H NMR (400 MHz, DMSO-d₆) δ 11.08 (d, J=3.6 Hz, 1H), 7.07-6.97 (m, 2H), 6.87 (d, J=8.1 Hz, 1H), 5.39-5.26 (m, 1H), 3.89 (s, 1H), 3.69-3.60 (m, 2H), 3.60-3.46 (m, 3H), 3.43 (s, 1H), 2.95 (d, J=6.9 Hz, 1H), 2.93-2.84 (m, 1H), 2.78-2.62 (m, 3H), 2.60 (s, 2H), 2.08 (s, 1H), 2.04-1.96 (m, 1H), 1.54 (d, J=4.0 Hz, 3H), 1.37 (d, J=3.8 Hz, 9H). LC/MS (ESI, m/z): [(M+1)]⁺=457.15.

The following intermediates in Table 27 were synthesized according to the above Procedure:

TABLE 27

| Inter- | | | | |
|---|---|---|---|---|
| medi- | | | MS: | |
| ate | Structure | Name | [(M + 1)]⁺ | ¹H NMR |
| R9 | 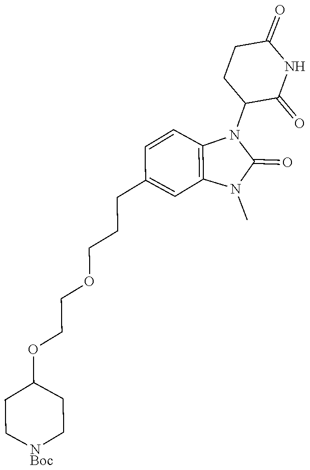 | tert-butyl 4-(2-[3- [1-(2,6- dioxopiperidin-3- yl)-3-methyl-2- oxo-1,3- benzodiazol-5- yl]propoxy]ethoxy) piperidine-1- carboxylate | 545.35 | (400 MHz, CD₃OD) δ 7.08-6.94 (m, 3H), 5.38-5.27 (m, 1H), 4.65-4.53 (m, 1H), 3.80-3.70 (m, 2H), 3.69- 3.63 (m, 2H), 3.62-3.58 (m, 2H), 3.50 (t, J = 6.4 Hz, 2H), 3.43 (s, 3H), 3.14 (t, J = 11.9 Hz, 2H), 3.01-2.79 (m, 3H), 2.77 (t, J = 6.3 Hz, 2H), 2.24- 2.12 (m, 1H), 1.98-1.80 (m, 4H), 1.46 (s, 11H) |
| R10 | 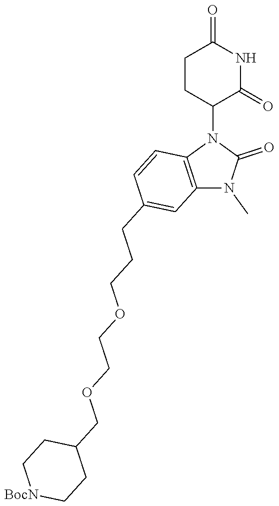 | tert-butyl 4-[(2-[3- [1-(2,6- dioxopiperidin-3- yl)-3-methyl-2- oxo-1,3- benzodiazol-5- yl]propoxy]ethoxy) methyl]piperidine- 1-carboxylate | 559.35 | (400 MHz, DMSO-d₆) δ 11.08 (s, 1H), 7.12-6.90 (m, 2H), 6.90-6.85 (m, 1H), 5.40-5.30 (m, 1H), 3.92 (d, J = 13.0 Hz, 2H), 3.39 (dt, J = 7.9, 5.8 Hz, 2H), 3.33 (d, J = 2.4 Hz, 6H), 3.25 (t, J = 6.8 Hz, 3H), 2.97-2.84 (m, 1H), 2.74-2.65 (m, 6H), 2.08-1.96 (m, 1H), 1.87-1.76 (m, 1H), 1.74- 1.59 (m, 4H), 1.39 (d, J = 4.7 Hz, 9H), 1.05-0.95 (m, 2H) |
| R11 | 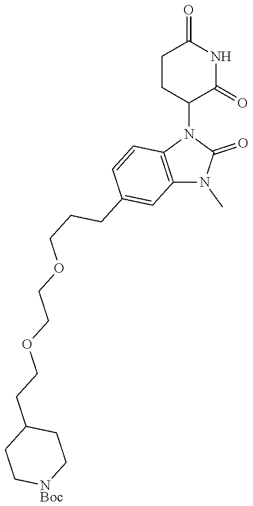 | tert-butyl 4-[2-(2- [3-[1-(2,6- dioxopiperidin-3- yl)-3-methyl-2- oxo-1,3- benzodiazol-5- yl]propoxy]ethoxy) ethyl]piperidine- 1-carboxylate | 573.40 | (400 MHz, DMSO-d₆) δ 11.08 (s, 1H), 7.06-6.97 (m, 2H), 6.90-6.85 (m, 1H), 5.40-5.30 (m, 1H), 3.89 (d, J = 13.0 Hz, 2H), 3.48-3.36 (m, 6H), 3.35-3.30 (m, 5H), 2.97-2.84 (m, 1H), 2.78-2.62 (m, 3H), 2.62-2.58 (m, 1H), 2.06-1.96 (m, 1H), 1.87- 1.76 (m, 2H), 1.66-1.58 (m, 2H), 1.53-1.49 (m, 1H), 1.45-1.40 (m, 2H), 1.38 (s, 9H), 1.01-0.95 (m 2H) |
| R12 | 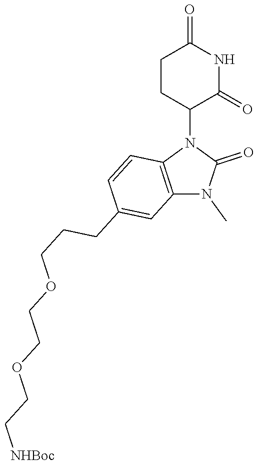 | tert-butyl N-[2-(2- [3-[1-(2,6- dioxopiperidin-3- yl)-3-methyl-2- oxo-2,3-dihydro- 1H-1,3- benzodiazol-5- yl]propoxy]ethoxy) ethyl]carbamate | 527.40 [(M + 23)]⁺ | (400 MHz, CDCl₃) δ 8.51 (s, 1H), 6.91 (d, J = 8.0 Hz, 1H), 6.88 (s, 1H), 6.73 (d, J = 8.0 Hz, 1H), 5.29-5.16 (m, 1H), 5.04 (s, 1H), 3.70-3.60 (m, 2H), 3.59-3.54 (m, 4H), 3.49 (t, J = 6.4 Hz, 2H), 3.43 (s, 3H), 3.34 (d, J = 5.7 Hz, 2H), 2.93-2.86 (m, 2H), 2.80- 2.70 (m, 3H), 2.27-2.18 (m, 1H), 2.00-1.90 (m, 2H), 1.44 (s, H) |
| R13 | 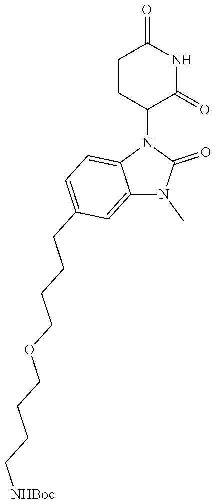 | tert-butyl N-(4-[4- [1-(2,6- dioxopiperidin-3- yl)-3-methyl-2- oxo-2,3-dihydro- 1H-1,3- benzodiazol-5- yl]butoxy]butyl)car- bamate | 503.35 | (400 MHz, CDCl₃) δ 8.12 (s, 1H), 6.95-6.89 (m, 1H), 6.87 (d, J = 1.5 Hz, 1H), 6.73 (d, J = 8.0 Hz, 1H), 5.28-5.17 (m, 1H), 4.67 (s, 1H), 3.47- 3.39 (m, 7H), 3.15 (d, J = 6.1 Hz, 2H), 3.05-2.95 (m, 1H), 2.92-2.85 (m, 1H), 2.83-2.71 (m, 1H), 2.70 (t, J = 7.6 Hz, 2H), 2.30-2.20 (m, 1H), 1.77-1.54 (m, 4H), 1.46 (s, 9H) |
| R14 | 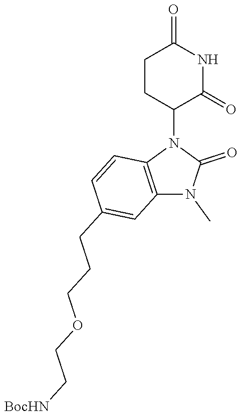 | tert-butyl N-(2-[3- [1-(2,6- dioxopiperidin-3- yl)-3-methyl-2- oxo-2,3-dihydro- 1H-1,3- benzodiazol-5- yl]propoxy]ethyl)car- bamate | 483.40 [(M + 23)]⁺ | (400 MHz, DMSO-d₆) δ 11.10 (s, 1H), 7.07-6.97 (m, 2H), 6.91-6.78 (m, 2H), 5.40-5.30 (m, 1H), 3.39 (d, J = 6.4 Hz, 4H), 3.33 (s, 3H), 3.14- 3.05 (m, 2H), 2.95-2.86 (m, 1H), 2.64 (d, J = 6.8 Hz, 3H), 2.00 (s, 1H), 1.85-1.78 (m, 2H), 1.38 (s, 9H), 1.25- 1.13 (m, 1H) |
| R15 | 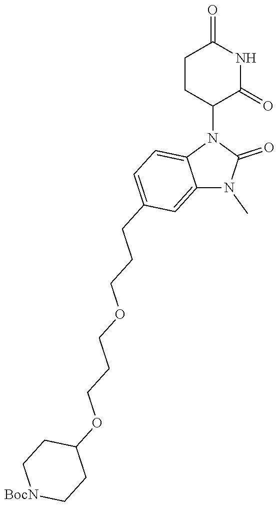 | tert-butyl 4-(3-[3- [1-(2,6- dioxopiperidin-3- yl)-3-methyl-2- oxo-2,3-dihydro- 1H-1,3- benzodiazol-5- yl]propoxy] propoxy)piperidine- 1-carboxylate | 557.35 [(M − 1)]⁻ | (400 MHz, DMSO-d₆) δ 11.09 (s, 1H), 7.12-6.96 (m, 2H), 6.86 (d, J = 7.8 Hz, 1H), 5.40-5.30 (m, 1H), 3.65- 3.60 (m, 1H), 3.49-3.33 (m, 9H), 3.11-2.81 (m, 3H), 2.79-2.53 (m, 4H), 2.07-1.92 (m, 1H), 1.80-1.70 (m, 6H), 1.40-1.35 (m, 13H) |
| R16 | 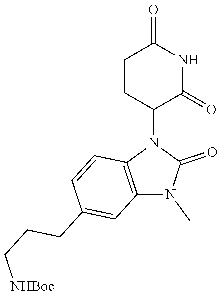 | tert-butyl N-[3-[1- (2,6- dioxopiperidin-3- yl)-3-methyl-2- oxo-2,3-dihydro- 1H-1,3- benzodiazol-5- yl]propyl]carbamate | 557.35 [(M − 100)]⁺ | (400 MHz, CDCl₃) δ 8.47 (s, 1H), 6.90 (d, J = 8.0 Hz, 1H), 6.89 (s, 1H), 6.71 (d, J = 8.0 Hz, 1H), 5.28-5.15 (m, 1H), 4.62 (s, 1H), 3.43 (s, 3H), 3.17 (s, 2H), 3.00-2.90 (m, 1H), 2.88- 2.82 (m, 1H), 2.81-2.72 (m, 1H), 2.69 (t, J = 7.5 Hz, 2H), 2.25-2.18 (m, 1H), 1.88-1.77 (m, 2H), 1.47 (s, 9H) |
| R17 | 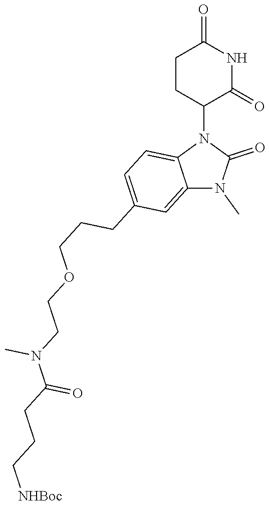 | tert-butyl N-[3-[(2- [3-[1-(2,6- dioxopiperidin-3- yl)-3-methyl-2- oxo-2,3-dihydro- 1H-1,3- benzodiazol-5- yl]propoxy]ethyl)(meth- yl)carbamoyl] propyl]carbamate | 560.30 | (400 MHz, DMSO-d₆) δ 11.10 (s, 1H), 7.06-6.97 (m, 2H), 6.90-6.79 (m, 2H), 5.40-5.30 (m, 1H), 3.56- 3.36 (m, 5H), 3.33 (s, 3H), 2.99 (s, 1H), 2.96-2.87 (m, 3H), 2.84 (s, 2H), 2.70-2.60 (m, 4H), 2.35 (t, J = 7.3 Hz, 1H), 2.27 (t, J = 7.4 Hz, 1H), 2.07- 1.94 (m, 1H), 1.81 (t, J = 7.3 Hz, 2H), 1.65-1.55 (m, 2H), 1.36 (d, J = 3.6 Hz, 9H) |
| R18 | 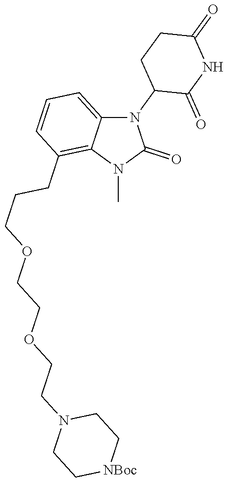 | tert-butyl 4-[2-(2- [3-[1-(2,6- dioxopiperidin-3- yl)-3-methyl-2- oxo-1,3- benzodiazol-4- yl]propoxy]ethoxy) ethyl]piperazine- 1-carboxylate | 574.35 | (400 MHz, DMSO-d₆) δ 11.09 (s, 1H), 7.01-6.92 (m, 2H), 6.87-6.84 (m, 1H), 5.40-5.30 (m, 1H), 3.59- 3.50 (m, 8H), 3.46 (t, J = 6.1 Hz, 2H), 3.33-3.25 (m, 5H), 3.00-2.83 (m, 3H), 2.78-2.65 (m, 1H), 2.65-2.58 (m, 1H), 2.37 (t, J = 5.0 Hz, 4H), 2.00 (m, 1H), 1.89-1.78 (m, 2H), 1.40- 1.35 (m, 11H) |

### INTERMEDIATE R19: tert-butyl 4-(3-[3-[1-(2,6-dioxopiperidin-3-yl)-3-methyl-2-oxo-1,3-benzodiazol-5-yl]propoxy]propyl)piperazine-1-carboxylate

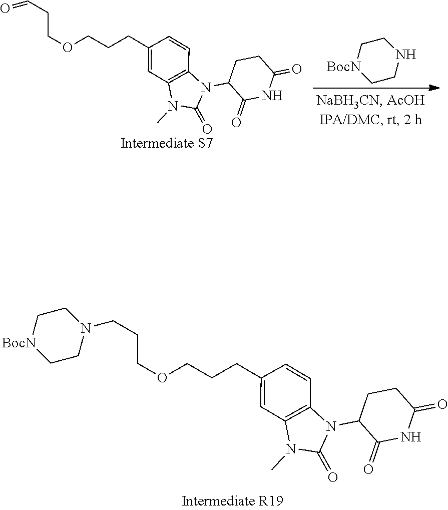 [SMILES: Cn1c(=O)n(C2CCC(=O)NC2=O)c2ccc(CCCOCCC=O)cc21 ; CC(C)(C)OC(=O)N1CCNCC1 ; Cn1c(=O)n(C2CCC(=O)NC2=O)c2ccc(CCCOCCCN3CCN(C(=O)OC(C)(C)C)CC3)cc21]

To a stirred solution of 3-[3-[1-(2,6-dioxopiperidin-3-yl)-3-methyl-2-oxo-1,3-benzodiazol-5-yl]propoxy]propanal (350 mg, 0.94 mmol) and tert-butyl piperazine-1-carboxylate (175 mg, 0.94 mmol) in IPA (10.0 mL) and DCM (5.00 mL) was added AcOH (113 mg, 1.88 mmol) at room temperature. The resulting solution was stirred for 15 min followed by the addition of NaBH₃CN (118 mg, 1.88 mmol) at room temperature. The resulting solution was stirred for 2 h at room temperature. The reaction solution was concentrated under reduced pressure. The residue was purified by reversed phase flash chromatography with the following conditions: Column: WelFlash™ C18-I, 20-40 um, 330 g; Eluent A: water (plus 10 mmol/L NH₄HCO₃); Eluent B: ACN; Gradient: 25%-45% B in 25 min; Flow rate: 80 mL/min; Detector: UV 220/254 nm; desired fractions were collected at 45% B and concentrated under reduced pressure to afford tert-butyl 4-(3-[3-[1-(2,6-dioxopiperidin-3-yl)-3-methyl-2-oxo-1,3-benzodiazol-5-yl]propoxy]propyl)piperazine-1-carboxylate (400 mg, 79%) as a white solid. ¹H NMR (400 MHz, DMSO-d₆) δ 11.08 (s, 1H), 7.10-6.96 (m, 2H), 6.90-6.86 (m, 1H), 5.40-5.30 (m, 1H), 3.90 (s, 2H), 3.46-3.38 (m, 5H), 3.33 (s, 3H), 3.25-2.77 (m, 7H), 2.77-2.58 (m, 4H), 2.08 (s, 1H), 2.06-1.96 (m, 1H), 1.93-1.76 (m, 4H), 1.42 (s, 9H). LC/MS (ESI, m/z): [(M+1)]⁺=544.35

### INTERMEDIATE W1: 3-(3-methyl-2-oxo-5-[3-[1-(piperidin-4-yl)azetidin-3-yl]propyl]-1,3-benzodiazol-1-yl)piperidine-2,6-dione hydrochloride

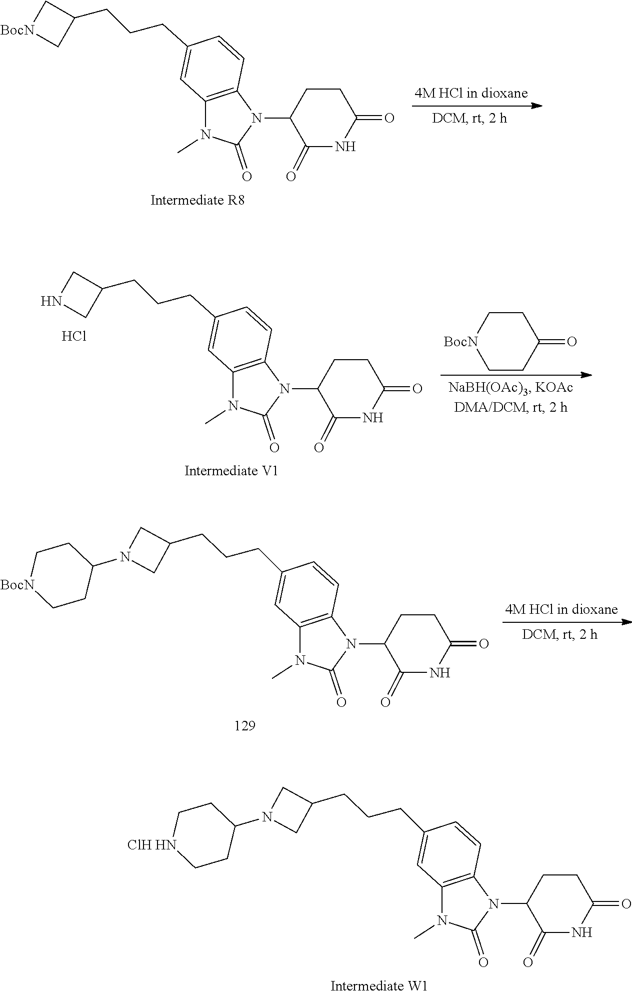

### Step 1: 3-[5-[3-(azetidin-3-yl)propyl]-3-methyl-2-oxo-1,3-benzodiazol-1-yl]piperidine-2,6-dione hydrochloride

To a stirred solution of tert-butyl 3-[3-[1-(2,6-dioxopiperidin-3-yl)-3-methyl-2-oxo-1,3-benzodiazol-5-yl]propyl]azetidine-1-carboxylate (200 mg, 0.44 mmol) in 1,4-dioxane (4.00 mL) was added HCl (4 M in 1,4-dioxane, 4.00 mL) dropwise at room temperature under nitrogen atmosphere. The resulting mixture was stirred for 2 h at room temperature. The resulting mixture was concentrated under vacuum. This resulted in 3-[5-[3-(azetidin-3-yl)propyl]-3-methyl-2-oxo-1,3-benzodiazol-1-yl]piperidine-2,6-dione hydrochloride (200 mg, crude) as a white solid. ¹H NMR (400 MHz, CD₃OD) δ 7.10-7.01 (m, 2H), 6.99-6.95 (m, 1H), 4.05-3.96 (m, 2H), 3.71-3.62 (m, 2H), 3.43 (s, 3H), 2.97-2.88 (m, 2H), 2.86-2.78 (m, 2H), 2.72 (s, 1H), 2.70 (s, 1H), 2.21-2.12 (m, 1H), 1.70 (q, J=8.0, 7.4 Hz, 2H), 1.65-1.58 (m, 2H). LC/MS (ESI, m/z): [(M+1)]⁺=357.25.

The following intermediates in Table 27 were synthesized according to the above procedure:

TABLE 27

| | | | MS: | |
|---|---|---|---|---|
| Intermediate | Structure | Name | [(M + 1)]⁺ | ¹H NMR |
| V2 | 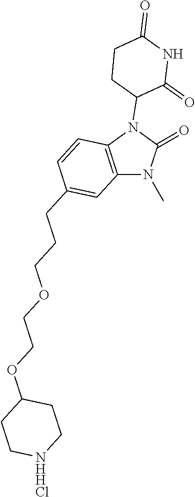 | 3-(3-methyl-2-oxo- 5-[3-[2-(piperidin-4- yloxy)ethoxy]propyl]- 1,3-benzodiazol- 1-yl)piperidine-2,6- dione hydrochloride | 445.25 | (400 MHz, CD₃OD) δ 7.09-7.02 (m, 2H), 6.99 (m, 1H), 5.40-5.30 (m, 1H), 3.81-3.57 (m, 7H), 3.52 (t, J = 6.4 Hz, 2H), 3.43 (d, J = 1.3 Hz, 3H), 3.20-3.10 (m, 2H), 3.04- 2.89 (m, 1H), 2.89-2.74 (m, 4H), 2.03 (d, J = 6.1 Hz, 1H), 2.03-1.95 (m, 2H), 1.94-1.83 (m, 4H) |
| V3 | 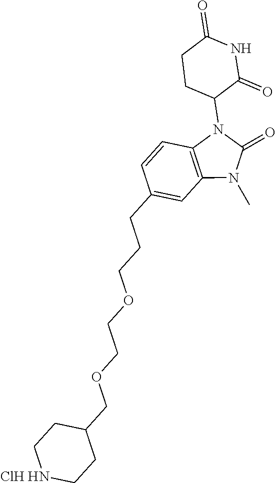 | 3-(3-methyl-2-oxo- 5-[3-[2-(piperidin-4- ylmethoxy)ethoxy] propyl]-1,3- benzodiazol-1- yl)piperidine-2,6- dione hydrochloride | 495.35 | (400 MHz, DMSO-d₆) δ 11.08 (s, 1H), 8.50 (s, 1H), 7.06-6.98 (m, 2H), 6.97 (s, 1H), 5.40-5.30 (m, 1H), 3.49 (d, J = 5.1 Hz, 6H), 3.31- 3.20 (m, 5H), 2.90-2.80 (m, 4H), 2.77-2.58 (m, 5H), 2.05-1.95 (m, 1H), 1.85-1.75 (m, 6H), 1.40-1.30 (m, 3H) |
| V4 | 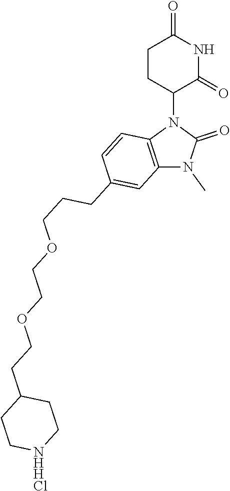 | 3-[3-methyl-2-oxo- 5-(3-[2-[2- (piperidin-4- yl)ethoxy]ethoxy] propyl)-1,3- benzodiazol-1- yl]piperidine-2,6- dione hydrochloride | 473.35 | (400 MHz, DMSO-d₆) δ 11.08 (s, 1H), 8.50 (s, 1H), 7.07-6.99 (m, 2H), 6.90-6.85 (m, 1H), 5.40-5.30 (m, 1H), 3.45-3.35 (m, 5H), 3.33 (s, 3H), 3.20 (d, J = 12.6 Hz, 5H), 2.98-2.84 (m, 1H), 2.82-2.75 (m, 2H), 2.74-2.57 (m, 4H), 2.05-1.95 (m, 1H), 1.85-1.75 (m, 4H), 1.70- 1.60 (m, 1H), 1.50-1.40 (m, 2H), 1.35-1.22 (m, 2H) |
| V5 | 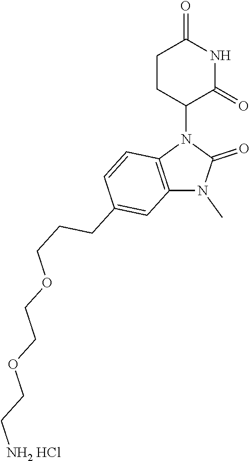 | 3-(5-[3-[2-(2- Aminoethoxy)ethoxy] propyl]-3-methyl- 2-oxo-2,3-dihydro- 1H-1,3- benzodiazol-1- yl)piperidine-2,6- dione hydrochloride | 423.25 [(M + 23)]⁺ | (400 MHz, DMSO-d₆) δ 11.10 (s, 1H), 8.05 (s, 2H), 7.07-6.99 (m, 2H), 6.90-6.85 (m, 1H), 5.40-5.30 (m, 1H), 3.63 (t, J = 5.3 Hz, 2H), 3.59-3.50 (m, 2H), 3.43-3.39 (m, 2H), 3.37 (d, J = 6.3 Hz, 2H), 3.03- 2.85 (m, 3H), 2.79-2.58 (m, 4H), 2.04-1.94 (m, 1H), 1.89-1.75 (m, 2H), 1.09 (t, J = 7.0 Hz, 1H) |
| V6 | 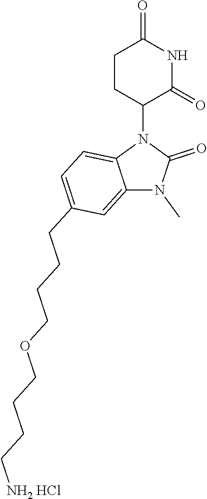 | 3-[5-[4-(4- aminobutoxy)butyl]- 3-methyl-2-oxo- 2,3-dihydro-1H- 1,3-benzodiazol-1- yl]piperidine-2,6- dione | 403.25 | (400 MHz, DMSO-d₆) δ 11.08 (s, 1H), 7.86 (s, 3H), 7.05-6.98 (m, 2H), 6.87(d, J = 8.0 Hz, 1H), 5.40- 5.30 (m, 1H), 3.74-3.63 (m, 1H), 3.57 (s, 1H), 3.52-3.48 (m, 1H), 3.42-3.33 (m, 4H), 2.97-2.85 (m, 1H), 2.82-2.59 (m, 6H), 2.06-1.96 (m, 1H), 1.67-1.48 (m, 8H) |
| N7 | 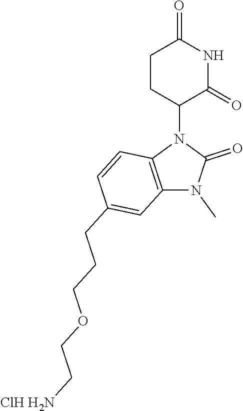 | 3-[5-[3-(2- aminoethoxy)propyl]- 3-methyl-2-oxo- 2,3-dihydro-1H- 1,3-benzodiazol-1- yl]piperidine-2,6- dione hydrochloride | 361.25 | (400 MHz, DMSO-d₆) δ 11.12 (s, 1H), 7.85 (s, 3H), 7.12-6.99 (m, 2H), 6.91-6.88 (m, 1H), 5.40-5.30 (m, 1H), 3.58-3.54 (m, 3H), 3.45 (t, J = 6.5 Hz, 2H), 3.33 (s, 3H), 2.99 (q, J = 5.5 Hz, 2H), 2.95-2.90 (m, 1H), 2.74-2.58 (m, 4H), 2.04- 1.95 (m, 1H), 1.94-1.80 (m, 2H) |
| V8 | 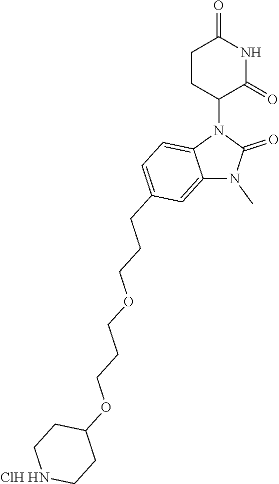 | 3-(3-methyl-2-oxo- 5-[3-[3-(piperidin-4- yloxy)propoxy]propyl]- 2,3-dihydro-1H-1,3- benzodiazol-1- yl)piperidine- 2,6-dione hydrochloride | 459.30 | (400 MHz, DMSO-d₆) δ 11.10 (s, 1H), 8.75 (d, J = 27.4 Hz, 2H), 7.06-6.98 (m, 2H), 6.90-6.85 (m, 1H), 5.40-5.30 (m, 1H), 3.56-3.36 (m, 9H), 3.11 (s, 2H), 3.00-2.87 (m, 3H), 2.77-2.59 (m, 4H), 2.05- 1.89 (m, 3H), 1.80-1.70 (m, 7H) |
| V9 | 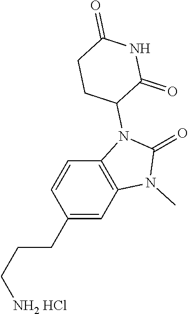 | 3-[5-(3- Aminopropyl)-3- methyl-2-oxo-2,3- dihydro-1H-1,3- benzodiazol-1- yl]piperidine-2,6- dione hydrochloride | 317.15 | (400 MHz, DMSO-d₆) δ 11.10 (s, 1H), 8.01 (s, 2H), 7.12-7.01 (m, 2H), 6.96-6.81 (m, 1H), 5.40-5.30 (m, 1H), 3.34 (s, 3H), 2.95-2.90 (m, 1H), 2.80-2.74 (m, 2H), 2.73- 2.57 (m, 3H), 2.06-1.96 (m, 1H), 1.92-1.88 (m, 2H), 1.37 (d, J = 9.4 Hz, 1H) |
| V10 | 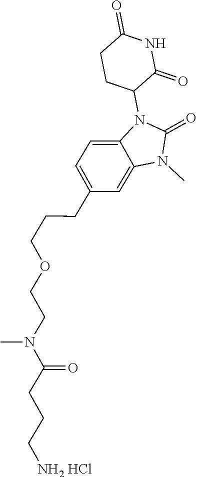 | 4-Amino-N-(2-[3- [1-(2,6- dioxopiperidin-3- yl)-3-methyl-2-oxo- 2,3-dihydro-1H- 1,3-benzodiazol-5- yl]propoxy]ethyl)- N- methylbutanamide hydrochloride | 460.35 | (400 MHz, DMSO-d₆) δ 11.10 (s, 1H), 8.03 (s, 2H), 7.11-7.00 (m, 2H), 6.88-6.84 (m, 1H), 5.40-5.30 (m, 1H), 3.55-3.47 (m, 3H), 3.45- 3.38 (m, 3H), 3.33 (d, J = 1.1 Hz, 2H), 3.03-2.92 (m, 2H), 2.86 (s, 2H), 2.80 (q, J = 6.4 Hz, 2H), 2.76- 2.67 (m, 1H), 2.67-2.53 (m, 3H), 2.50-2.39 (m, 3H), 2.02-1.98 (m, 1H), 1.85-1.75 (m, 4H) |
| V11 | 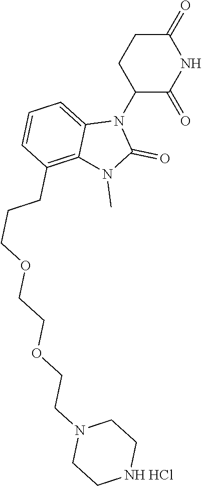 | 3-(3-methyl-2-oxo- 4-(3-(2-(2- (piperazin-1- yl)ethoxy)ethoxy) propyl)-2,3-dihydro- 1H- benzo[d]imidazol- 1-yl)piperidine-2,6- dione hydrochloride | 474.30 | (400 MHz, DMSO-d₆) δ 11.11 (s, 1H), 7.00-6.91 (m, 2H), 6.86-6.82 (m, 1H), 5.40-5.30 (m, 1H), 3.55- 3.44 (m, 8H), 3.45-3.40 (m, 4H), 3.34 (s, 3H), 3.01-2.82 (m, 3H), 2.78-2.64 (m, 1H), 2.64-2.56 (m, 1H), 2.40-2.30 (m, 4H), 2.10-2.00 (m, 1H), 1.88-1.76 (m, 2H), 1.40- 1.35 (m, 2H) |
| V12 | 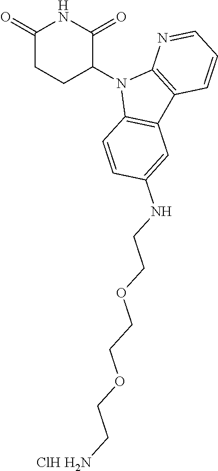 | 3-[6-([2-[2-(2- aminoethoxy)ethoxy] ethyl]amino)-9H- pyrido[2,3-b]indol- 9-yl]piperidine-2,6- dione hydrochloride | 426.15 | (400 MHz, DMSO-d₆) δ 11.19 (s, 1H), 8.60 (d, J = 7.6 Hz, 1H), 8.50 (d, J = 4.8 Hz, 1H), 7.96 (s, 3H), 7.67 (d, J = 55.8 Hz, 1H), 7.36- 7.32 (m, 1H), 6.10 (s, 1H), 3.63 (d, J = 4.8 Hz, 8H), 3.56 (d, J = 8.0 Hz, 4H), 3.06 (d, J = 13.7 Hz, 2H), 3.00-2.95 (m, 2H), 2.73 (d, J = 14.7 Hz, 1H), 2.16 (d, J = 8.2 Hz, 1H) |
| V13 | 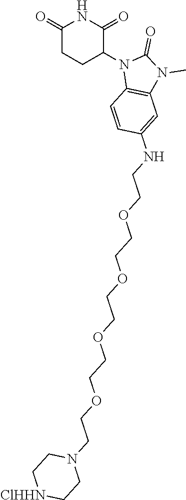 | 3-[3-methyl-2-oxo- 5-[15-(piperazin-1- yl)-4,7,10,13- tetraoxa-1- azapentadecan-1- yl]-2,3-dihydro-1H- 1,3-benzodiazol-1- yl]piperidine-2,6- dione hydrochloride | 563.30 | (400 MHz, DMSO-d₆) δ 11.99 (s, 1H), 11.11 (s, 1H), 10.29 (s, 1H), 7.71 (s, 1H), 7.59 (s, 1H), 7.28 (t, J = 7.7 Hz, 1H), 5.55-5.46 (m, 1H), 3.82 (s, 3H), 3.72 (t, J = 5.6 Hz, 2H), 3.56-3.47 (m, 12H), 3.47-3.35 (m, 6H), 2.94 (s, 2H), 2.76-2.54 (m, 6H), 2.06 (s, 4H) |
| V16 | 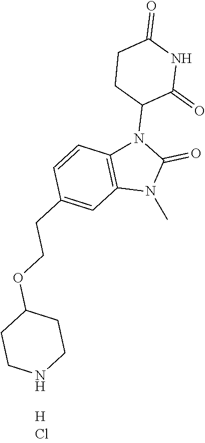 [SMILES: Cn1c(=O)n(C2CCC(=O)NC2=O)c2ccc(CCOC3CCNCC3)cc21] | 3-[3-methyl-2-oxo- 5-[2-(piperidin-4- yloxy)ethyl]-1,3- benzodiazol-1- yl]piperidine-2,6- dione hydrochloride | 387.30 | (400 MHz, DMSO-d₆) δ 11.08 (s, 1H), 8.94-8.90 (m, 1H), 7.15-7.07 (m, 1H), 7.06-7.00 (m, 1H), 6.95- 6.90 (m, 1H), 5.40-5.30 (m, 1H), 3.70-3.60 (m, 3H), 3.33 (s, 3H), 3.07 (s, 2H), 2.98-2.80 (m, 5H), 2.77-2.56 (m, 2H), 2.05-1.89 (m, 3H), 1.74-1.62 (m, 2H) |
| V17 | 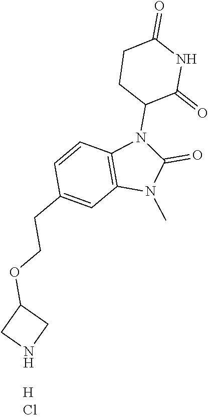 [SMILES: Cn1c(=O)n(C2CCC(=O)NC2=O)c2ccc(CCOC3CNC3)cc21] | 3-[5-[2-(azetidin-3- yloxy)ethyl]-3- methyl-2-oxo-1,3- benzodiazol-1- yl]piperidine-2,6- dione hydrochloride | 359.10 | (400 MHz, DMSO-d₆) δ 11.08 (s, 1H), 7.07 (s, 1H), 7.01 (d, J = 8.0 Hz, 1H), 6.90 (d, J = 7.9 Hz, 1H), 5.40-5.30 (m, 1H), 3.70-3.63 (m, 2H), 3.50 (t, J = 7.1 Hz, 2H), 3.35 (s, 3H), 2.95-2.90 (m, 1H), 2.72- 2.59 (m, 2H), 2.36 (d, J = 9.4 Hz, 3H), 2.30-2.22 (m, 2H), 2.10-2.05 (m, 1H) |
| V19 | 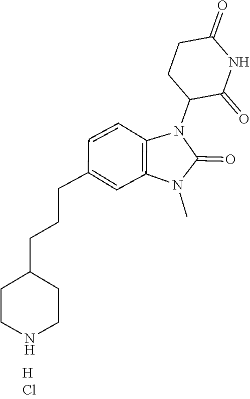 [SMILES: Cn1c(=O)n(C2CCC(=O)NC2=O)c2ccc(CCCC3CCNCC3)cc21] | 3-[3-methyl-2-oxo- 5-[3-(piperidin-4- yl)propyl]-1,3- benzodiazol-1- yl]piperidine-2,6- dione hydrochloride | 385.20 | (400 MHz, DMSO-d₆) δ 11.08 (s, 1H), 8.73 (s, 1H), 8.46 (s, 1H), 7.06-6.98 (m, 2H), 6.90-6.85 (m, 1H), 5.40-5.30 (m, 1H), 3.25-3.20 (m, 2H), 2.96-2.58 (m, 7H), 2.05- 1.94 (m, 1H), 1.85-1.74 (m, 2H), 1.67-1.48 (m, 3H), 1.30-1.20 (m, 4H) |
| V22 | 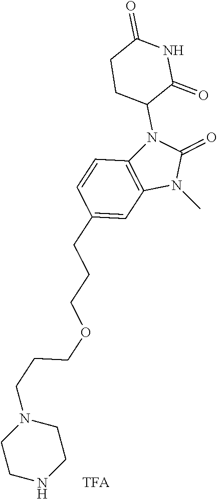 [SMILES: Cn1c(=O)n(C2CCC(=O)NC2=O)c2ccc(CCCOCCCN3CCNCC3)cc21] | 3-(3-methyl-2-oxo- 5-[3-[3-(piperazin-1- yl)propoxy]propyl]- 1,3-benzodiazol-1- yl)piperidine-2,6- dione; trifluoroacetaldehyde | 444.30 | (400 MHz, DMSO-d₆) δ 11.08 (s, 1H), 9.13 (br, 2H), 7.05-6.99 (m, 2H), 6.87 (d, J = 8.1 Hz, 1H), 5.35 (dd, J = 12.8, 5.3 Hz, 1H), 3.72- 3.64 (m, 2H), 3.63-3.54 (m, 8H), 3.51 (q, J = 2.1 Hz, 2H), 3.47-3.41 (m, 7H), 2.76-2.58 (m, 3H), 2.01 (d, J = 12.2 Hz, 1H), 1.94-1.77 (m, 4H) |
| V14 | 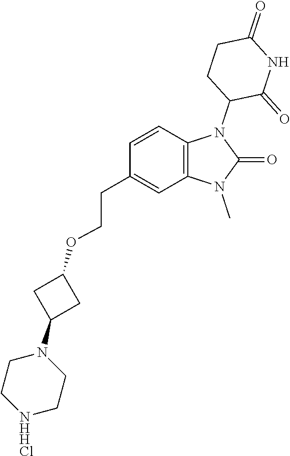 | 3-(3-methyl-2-oxo- 5-[2-[trans-3- (piperazin-1- yl)cyelobutoxy] ethyl]-1,3- benzodiazol-1- yl)piperidine-2,6- dione hydrochloride | 442.20 | N/A |
| V15 | 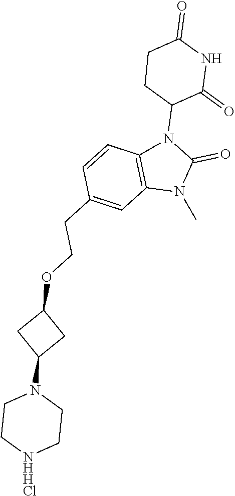 | 3-(3-methyl-2-oxo- 5-[2-[cis-3- (piperazin-1- yl)cyelobutoxy] ethyl]-1,3- benzodiazol-1- yl)piperidine-2,6- dione hydrochloride | 442.20 | N/A |
| V18 | 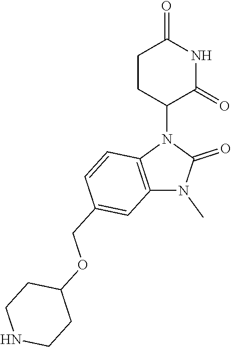 [SMILES: Cn1c(=O)n(C2CCC(=O)NC2=O)c2ccc(COC3CCNCC3)cc21] | 3-[3-methyl-2-oxo- 5-[(piperidin-4- yloxy)methyl]-1,3- benzodiazol-1- yl]piperidine-2,6- dione | 373.15 | N/A |
| V20 | 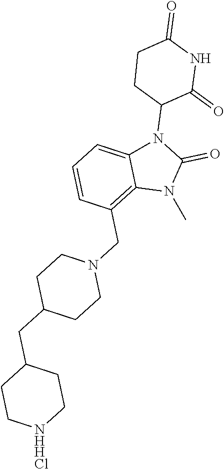 | 3-(3-methyl-2-oxo- 4-[[4-(piperidin-4- ylmethyl)piperidin- 1-yl]methyl]-1,3- benzodiazol-1- yl)piperidine-2,6- dione hydrochloride | 454.25 | N/A |
| V21 | 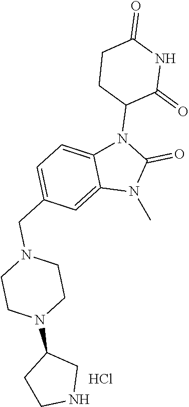 [SMILES: Cn1c(=O)n(C2CCC(=O)NC2=O)c2ccc(CN3CCN([C@@H]4CCNC4)CC3)cc21 ; Cl] | 3-[3-methyl-2-oxo- 5-([4-[(3R)- pyrrolidin-3- yl]piperazin-1- yl]methyl)-1,3- benzodiazol-1- yl]piperidine-2,6- dione hydrochloride | 427.20 | N/A |

### Step 2: tert-butyl 4-(3-[3-[1-(2,6-dioxopiperidin-3-yl)-3-methyl-2-oxo-1,3-benzodiazol-5-yl]propyl]azetidin-1-yl)piperidine-1-carboxylate

To a stirred solution of 3-[5-[3-(azetidin-3-yl)propyl]-3-methyl-2-oxo-1,3-benzodiazol-1-yl]piperidine-2,6-dione (156 mg, 0.44 mmol), tert-butyl 4-oxopiperidine-1-carboxylate (131 mg, 0.66 mmol) and KOAc (215 mg, 2.19 mmol) in DCM (6.00 mL) and DMA (2.00 mL) was added NaBH(OAc)₃ (464 mg, 2.19 mmol) at room temperature under nitrogen atmosphere. After stirring for additional 2 h the resulting mixture was concentrated under reduced pressure. The residue was purified by reversed phase flash chromatography with the following conditions: Column: Xselect CSH OBD Column 30×150 mm 5 um; Mobile Phase A: water (plus 0.1% FA), Mobile Phase B: ACN; Flow rate: 60 mL/min; Gradient: 5% B to 20% B in 7 min; Detector: UV 220/254 nm; Rt: 6.18 min. Desired fractions were collected and concentrated under reduced pressure to afford tert-butyl 4-(3-[3-[1-(2,6-dioxopiperidin-3-yl)-3-methyl-2-oxo-1,3-benzodiazol-5-yl]propyl]azetidin-1-yl)piperidine-1-carboxylate (60.0 mg, 25%) as a white solid. ¹H NMR (400 MHz, CD₃OD) δ 7.02 (d, J=9.6 Hz, 2H), 6.96 (d, J=8.2 Hz, 1H), 5.32 (m, 1H), 4.04 (d, J=13.7 Hz, 2H), 3.50 (t, J=7.5 Hz, 2H), 3.43 (s, 3H), 2.99-2.87 (m, 1H), 2.85-2.78 (m, 4H), 2.70 (t, J=6.8 Hz, 2H), 2.55-2.45 (m, 1H), 1.74 (d, J=12.9 Hz, 2H), 1.59 (s, 3H), 1.46 (s, 9H), 1.10-1.05 (m, 2H). LC/MS (ESI, m/z): [(M+1)]⁺=540.60.

The following intermediates in Table 28 were synthesized according to the above procedure:

TABLE 28

| Inter- | | | | |
|---|---|---|---|---|
| medi- | | | MS: | |
| ate | Structure | Name | [(M + 1)]⁺ | ¹H NMR |
| 129- 1 | 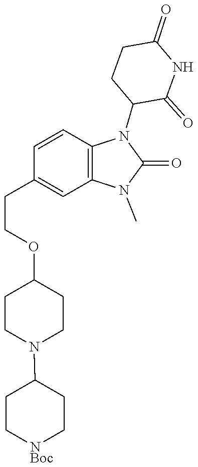 [SMILES: Cn1c(=O)n(C2CCC(=O)NC2=O)c2ccc(CCOC3CCN(C4CCN(C(=O)OC(C)(C)C)CC4)CC3)cc21] | tert-butyl 4-[2-[1- (2,6-dioxopiperidin- 3-yl)-3-methyl-2- oxo-1,3- benzodiazol-5- yl]ethoxy]-[1,4- bipiperidine]-1- carboxylate | 570.32 | (400 MHz, CDCl₃) δ 7.02-6.89 (m, 2H), 6.72 (d, J = 8.0 Hz, 1H), 5.30- 5.20 (m, 1H), 3.75-3.65 (m, 2H), 3.45 (s, 3H), 3.35-3.28 (m, 1H), 3.01-2.78 (m, 5H), 2.78-2.61 (m, 4H), 2.46 (t, J = 6.4 Hz, 1H), 2.29- 2.21 (m, 1H), 2.03 (s, 1H), 1.99-1.53 (m, 9H), 1.47 (s, 9H), 1.43 (s, 2H) |
| 129- 2 | 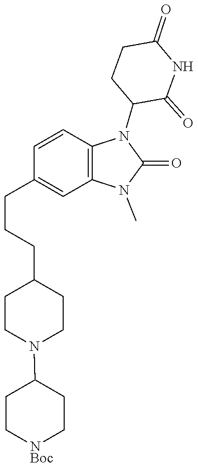 [SMILES: Cn1c(=O)n(C2CCC(=O)NC2=O)c2ccc(CCCC3CCN(C4CCN(C(=O)OC(C)(C)C)CC4)CC3)cc21] | tert-butyl 4-[3-[1- (2,6-dioxopiperidin- 3-yl)-3-methyl-2- oxo-1,3- benzodiazol-5- yl]propyl]-[1,4- bipiperidine]-1- carboxylate | 568.55 | (400 MHz, DMSO-d₆) δ 7.05-6.96 (m, 2H), 6.86 (m, 1H), 5.40-5.30 (m, 1H), 4.02-3.87 (m, 2H), 3.61 (t, J = 6.2 Hz, 1H), 2.95-2.85 (m, 1H), 2.79 (d, J = 10.9 Hz, 2H), 2.75-2.66 (m, 2H), 2.66-2.54 (m, 4H), 2.40-2.30 (m, 2H), 2.13-2.04 (m, 2H), 2.04- 1.95 (m, 1H), 1.71-1.52 (m, 6H), 1.39 (s, 9H), 1.30-1.20 (m, 5H), 1.10-1.00 (m, 2H) |
| 129- 3 | 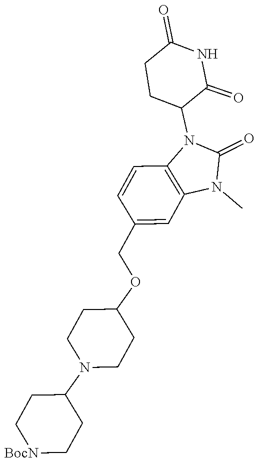 [SMILES: Cn1c(=O)n(C2CCC(=O)NC2=O)c2ccc(COC3CCN(C4CCN(C(=O)OC(C)(C)C)CC4)CC3)cc21] | tert-butyl 4-[[1-(2,6- dioxopiperidin-3- yl)-3-methyl-2-oxo- 1,3-benzodiazol-5- yl]methoxy]-[1,4- bipiperidine]-1- carboxylate | 556.35 | (400 MHz, DMSO-d₆) δ 7.21 (s, 1H), 7.17-7.07 (m, 2H), 5.40-5.30 (m, 1H), 4.62 (s, 2H), 4.19-4.09 (m, 2H), 3.52 (d, J = 4.3 Hz, 1H), 3.45 (d, J = 5.0 Hz, 3H), 2.98-2.59 (m, 8H), 2.54-2.44 (m, 1H), 2.39 (t, J = 10.7 Hz, 2H), 2.25-2.15 (m, 1H), 1.97 (d, J = 7.8 Hz, 2H), 1.90-1.85 (m, 2H), 1.67 (d, J = 7.1 Hz, 3H), 1.47 (s, 9H), 1.45-1.35 (m, 2H), 1.31 (s, 1H) |
| 129- 4 | 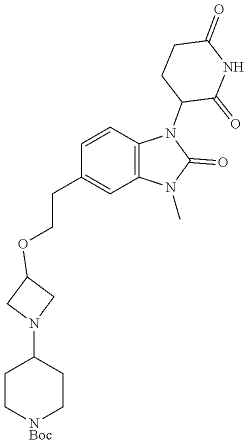 [SMILES: Cn1c(=O)n(C2CCC(=O)NC2=O)c2ccc(CCOC3CN(C4CCN(C(=O)OC(C)(C)C)CC4)C3)cc21] | tert-butyl 4-(3-[2-[1- (2,6-dioxopiperidin- 3-yl)-3-methyl-2- oxo-1,3- benzodiazol-5- yl]ethoxy]azetidin- 1-yl)piperidine-1- carboxylate | 542.30 | (400 MHz, DMSO-d₆) δ 7.08 (s, 1H), 7.02 (d, J = 7.9 Hz, 1H), 6.91 (d, J = 8.0 Hz, 1H), 4.08-4.06-3.97 (m, 1H), 3.68 (s, 2H), 3.53 (t, J = 7.1 Hz, 3H), 3.50-3.40 (m, 4H), 3.36 (s, 3H), 2.82 (t, J = 7.0 Hz, 3H), 2.73 (d, J = 6.7 Hz, 3H), 2.12 (s, 1H), 2.00 (s, 1H), 1.60-1.52 (m, 2H), 1.38 (s, 9H), 1.01 (d, J = 10.3 Hz, 2H) |

### Step 3: 3-(3-methyl-2-oxo-5-[3-[1-(piperidin-4-yl)azetidin-3-yl]propyl]-1,3-benzodiazol-1-yl)piperidine-2,6-dione hydrochloride

To a stirred solution of tert-butyl 4-(3-[3-[1-(2,6-dioxopiperidin-3-yl)-3-methyl-2-oxo-1,3-benzodiazol-5-yl]propyl]azetidin-1-yl)piperidine-1-carboxylate (80.0 mg, 0.15 mmol) in 1,4-dioxane (5.00 mL) was added HCl (4 M in 1,4-dioxane, 5.00 mL) dropwise at room temperature under nitrogen atmosphere. The resulting mixture was stirred for 2 h at room temperature. The resulting mixture was concentrated under reduced pressure. This resulted in 3-(3-methyl-2-oxo-5-[3-[1-(piperidin-4-yl)azetidin-3-yl]propyl]-1,3-benzodiazol-1-yl)piperidine-2,6-dione (50.0 mg, crude) as a white solid. LC/MS (ESI, m/z): [(M+1)]⁺=440.20.

The following intermediates in Table 29 were synthesized according to the above procedure:

TABLE 29

| Inter- | | | | |
|---|---|---|---|---|
| medi- | | | MS: | |
| ate | Structure | Name | [(M + 1)]⁺ | ¹H NMR |
| W2 | 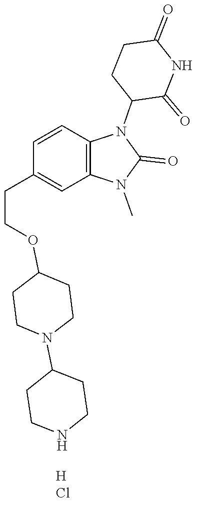 [SMILES: Cn1c(=O)n(C2CCC(=O)NC2=O)c2ccc(CCOC3CCN(C4CCNCC4)CC3)cc21] | 3-[5-(2-[[1,4- bipiperidin]-4- yloxy]ethyl)-3- methyl-2-oxo- 1,3-benzodiazol- 1-yl)piperidine- 2,6-dione hydrochloride | 470.30 | (400 MHz, CD₃OD) δ 7.18-6.99 (m, 3H), 5.45-5.37 (m, 1H), 3.80-3.70 (m, 3H), 3.53 (d, J = 13.5 Hz, 2H), 3.45 (d, J = 12.2 Hz, 3H), 3.12 (d, J = 11.2 Hz, 2H), 3.10-3.01 (m, 2H), 2.99-2.90 (m, 3H), 2.88-2.81 (m, 2H), 2.66-2.50 (m, 2H), 2.30-2.11 (m, 3H), 2.05 (s, 2H), 2.00 (s, 1H), 1.98-1.89 (m, 2H), 1.89- 1.73 (m, 2H) |
| W3 | 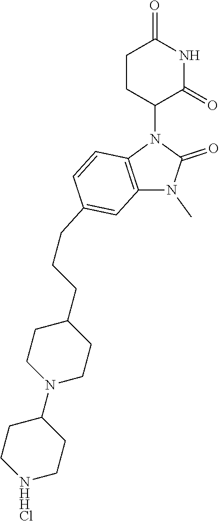 | 3-[5-(3-[[1,4- bipiperidin]-4- yl]propyl)-3- methyl-2-oxo- 1,3-benzodiazol- 1-yl]piperidine- 2,6-dione hydrochloride | 468.25 | (400 MHz, DMSO-d₆) δ 11.08 (s, 1H), 10.81 (s, 1H), 9.14 (s, 1H), 7.07-6.98 (m, 2H), 6.87 (m, 1H), 5.40-5.30 (m, 1H), 3.45-3.39 (m, 5H), 3.33 (s, 3H), 2.95-2.80 (m, 5H), 2.66-2.52 (m, 3H), 2.30-2.19 (m, 2H), 2.05-1.80 (m, 5H), 1.70-1.50 (m, 5H), 1.25 (d, J = 6.4 Hz, 2H) |
| W4 | 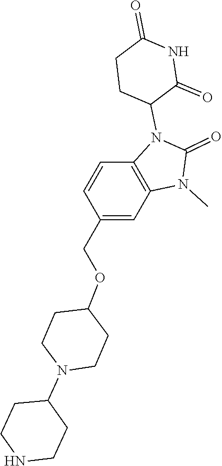 [SMILES: Cn1c(=O)n(C2CCC(=O)NC2=O)c2ccc(COC3CCN(C4CCNCC4)CC3)cc21] | 3-[5-([[1,4- bipiperidin]-4- yloxy]methyl)-3- methyl-2-oxo- 1,3-benzodiazol- 1-yl]piperidine- 2,6-dione | 456.25 | Crude to next step without further purification |
| W5 | 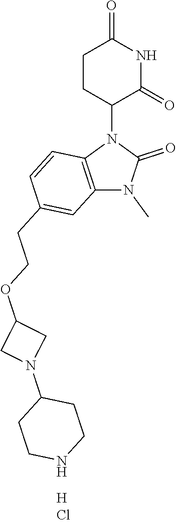 [SMILES: Cn1c(=O)n(C2CCC(=O)NC2=O)c2ccc(CCOC3CN(C4CCNCC4)C3)cc21] | 3-[3-methyl-2- oxo-5-(2-[[1- (piperidin-4- yl)azetidin-3- yl]oxy]ethyl)- 1,3-benzodiazol- 1-yl]piperidine- 2,6-dione hydrochloride | 442.35 | (400 MHz, DMSO-d₆) δ 11.09 (s, 1H), 10.80 (s, 1H), 9.11 (s, 1H), 7.05-6.92 (m, 2H), 6.90-6.85 (m, 1H), 4.08-4.02 (m, 1H), 3.68 (s, 2H), 3.53 (t, J = 7.1 Hz, 3H), 3.50-3.40 (m, 4H), 3.35 (s, 3H), 2.82 (t, J = 7.0 Hz, 3H), 2.73 (d, J = 6.7 Hz, 3H), 2.12 (s, 1H), 2.00 (s, 1H), 1.60- 1.52 (m, 2H), 1.01 (d, J = 10.3 Hz, 2H) |

### INTERMEDIATE T2: 5-[(3aS,4S,6aR)-2-oxo-hexahydro-1H-thieno[3,4-d]imidazol-4-yl]-N-[2-[2-(2-hydroxyethoxy)ethoxy]ethyl]pentanamide

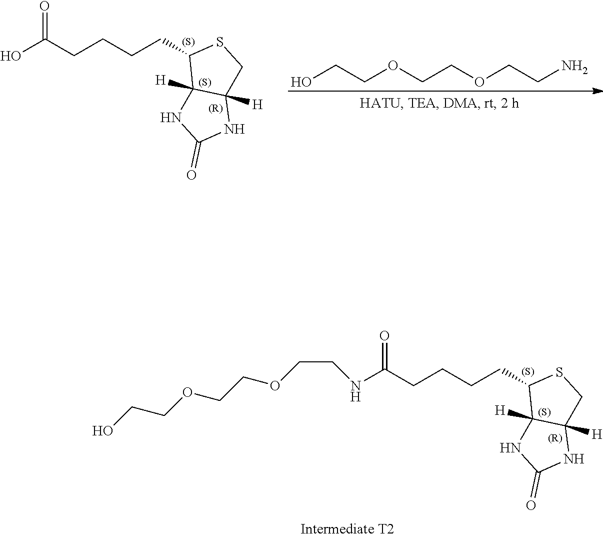

A solution of 5-[(3aS, 4S, 6aR)-2-oxo-hexahydro-1H-thieno[3,4-d]imidazol-4-yl]pentanoic acid (1.00 g, 4.09 mmol) in DMA (10.0 mL) was treated with HATU (2.02 g, 5.32 mmol) for 10 min at 0° C. under nitrogen atmosphere followed by the addition of TEA (1.71 mL, 16.9 mmol) and 2-[2-(2-aminoethoxy)ethoxy]ethan-1-ol (672 mg, 4.50 mmol) at 0° C. The resulting mixture was stirred for 2 h at room temperature and was concentrated under reduced pressure. The residue was re-dissolved in DCM (50.0 mL) and washed with brine (50.0 mL). The aqueous phase was concentrated under reduced pressure and the residue was purified by reversed phase flash chromatography with the following conditions: Column: WelFlash™ C18-I, 20-40 um, 330 g; Eluent A: water (plus 10 mmol/L FA); Eluent B: ACN; Gradient: 1%-20% B in 20 min; Flow rate: 80 mL/min; Detector: UV 220/254 nm; desired fractions were collected at 10% B and concentrated under reduced pressure to afford 5-[(3aS, 4S, 6aR)-2-oxo-hexahydro-1H-thieno[3,4-d]imidazol-4-yl]-N-[2-[2-(2-hydroxyethoxy)ethoxy]ethyl]pentanamide (1.17g, 76%) as a white solid. ¹H NMR (400 MHz, DMSO-d₆) δ 7.91 (t, J=5.6 Hz, 1H), 6.43 (s, 1H), 6.39 (s, 1H), 4.66 (s, 1H), 4.31 (m, 1H), 4.13 (m, 1H), 3.48 (d, J=5.1 Hz, 2H), 3.42 (s, 1H), 3.41 (s, 3H), 3.39 (s, 3H), 3.18 (q, J=5.9 Hz, 2H), 3.15-3.05 (m, 1H), 2.85-2.80 (m, 1H), 2.58 (d, J=12.4 Hz, 1H), 2.07 (t, J=7.4 Hz, 2H), 1.65-1.60 (m, 1H), 1.52-1.43 (m, 3H), 1.38-1.22 (m, 2H). LC/MS (ESI, m/z): [(M+1)]⁺=376.30

### INTERMEDIATE T3: 3-[5-[2-(3-hydroxycyclobutoxy)ethyl]-3-methyl-2-oxo-1,3-benzodiazol-1-yl]piperidine-2,6-dione

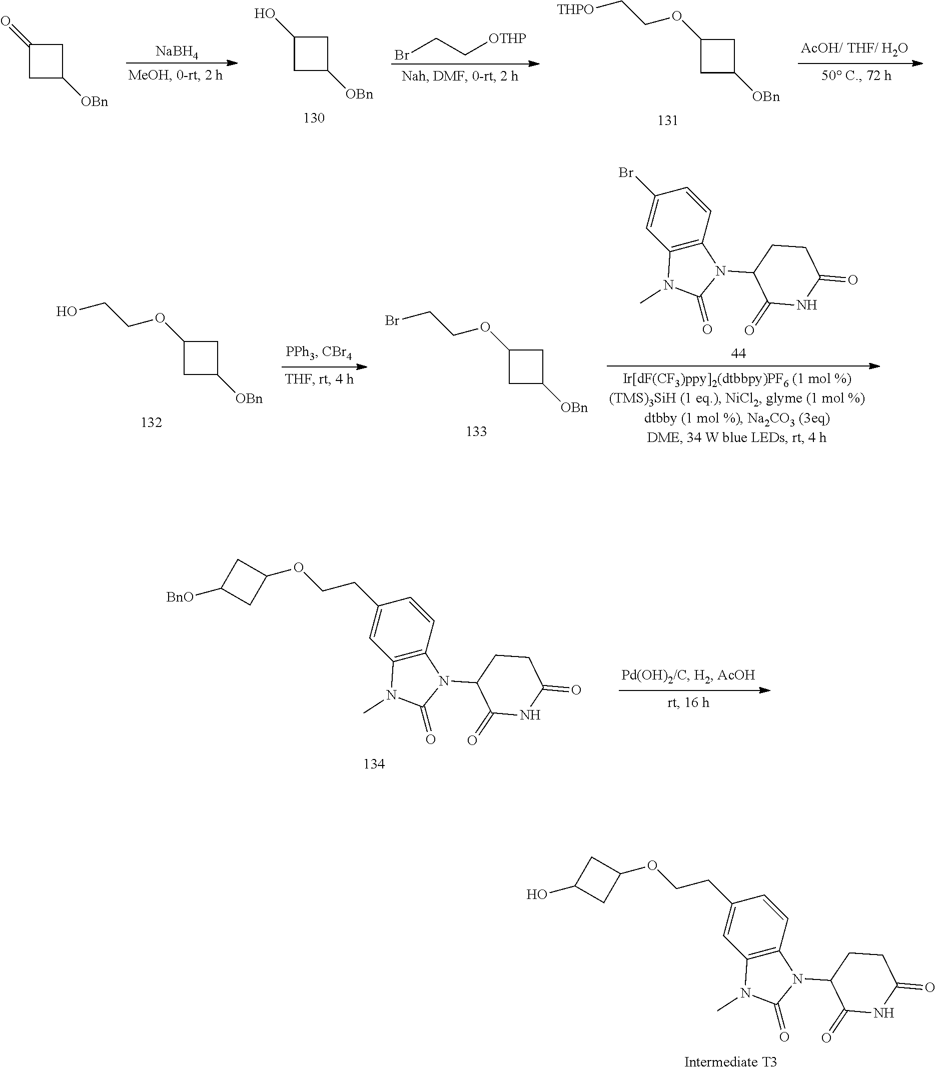

### Step 1: 3-(benzyloxy)cyclobutan-1-ol

To a stirred solution of 3-(benzyloxy)cyclobutan-1-one (11.0 g, 62.4 mmol) in MeOH (100 mL) was added NaBH4 (5.90 g, 156 mmol) in portions at 0° C. under nitrogen atmosphere. The solution was stirred for 2 h at 0° C. Upon completion, the reaction was quenched by the addition of water (50.0 mL) at 0° C. The resulting mixture was extracted with DCM (3×50.0 mL). The combined organic layers were dried over anhydrous Na₂SO₄. After filtration, the filtrate was concentrated under reduced pressure. The residue was purified by silica gel column chromatography, eluted with EtOAc: Petroleum ether (v:v=1:1) to afford 3-(benzyloxy)cyclobutan-1-ol (10.5 g, 94%) as a colorless oil. ¹H NMR (400 MHz, DMSO-d₆) δ 7.35-7.25 (m, 5H), 5.01 (d, J=6.6 Hz, 1H), 4.34 (s, 2H), 3.70-3.60 (m, 1H), 3.58-3.47 (m, 1H), 2.60-2.50 (m, 2H), 1.80-1.70 (m, 2H). LC/MS (ESI, m/z): [(M+18)]⁺=196.10

### Step2: 2-[2-[3-(benzyloxy)cyclobutoxy]ethoxy]oxane

To a stirred solution of 3-(benzyloxy)cyclobutan-1-ol (10.0 g, 56.1 mmol) in DMF (75.0 mL) was added NaH (5.61 g, 140 mmol, 60% dispersion in mineral oil) in portions at 0° C. under nitrogen atmosphere. The mixture was stirred for 30 min at 0° C. Then 2-(2-bromoethoxy)oxane (17.6 g, 84.2 mmol) was added. The mixture was stirred for 2 h at room temperature. Upon completion, the reaction was quenched by the addition of water (100 mL) at 0° C. The resulting mixture was extracted with EtOAc (2×100 mL). The combined organic layers were washed with water (50.0 mL) and dried over anhydrous Na₂SO₄. After filtration, the filtrate was concentrated under reduced pressure. The residue was purified by silica gel column chromatography, eluted with EtOAc:Petroleum ether (v:v=1:3) to afford 2-[2-[3-(benzyloxy)cyclobutoxy]ethoxy]oxane (16.0 g, 93%) as a colorless oil. ¹H NMR (400 MHz, DMSO-d₆) δ 7.44-7.15 (m, 5H), 4.60-4.54 (m, 1H), 4.36 (s, 2H), 3.78-3.57 (m, 4H), 3.50-3.37 (m, 4H), 2.62-2.56 (m, 2H), 1.81-1.57 (m, 4H), 1.51-1.37 (m, 4H). LC/MS (ESI, m/z): [(M+18)]⁺=324.15

### Step 3: 2-[3-(benzyloxy) cyclobutoxy]ethanol

A solution of 2-[2-[3-(benzyloxy)cyclobutoxy]ethoxy]oxane (17.0 g, 55.5 mmol) in AcOH (40.0 mL)/H₂O (10.0 mL)/THF (20.0 mL) was stirred for 72 h at 50° C. under nitrogen atmosphere. Upon completion, the solution was concentrated under reduced pressure and the residue was purified by silica gel column chromatography, eluted with EtOAc:Petroleum ether (v:v=1:1) to afford 2-[3-(benzyloxy)cyclobutoxy]ethanol (9.50 g, 77%) as a colorless oil. ¹H NMR (400 MHz, DMSO-d₆) δ 7.39-7.24 (m, 5H), 4.36 (s, 2H), 3.71-3.62 (m, 1H), 3.62-3.55 (m, 1H), 3.47 (t, J=5.3 Hz, 2H), 3.30 (t, J=5.3 Hz, 2H), 2.60-2.55 (m, 2H), 1.80-1.84 (m, 2H). LC/MS (ESI, m/z): [(M+18)]⁺=240.20

### Step 4: [[3-(2-bromoethoxy)cyclobutoxy]methyl]benzene

To a stirred solution of 2-[3-(benzyloxy)cyclobutoxy]ethanol (8.00 g, 36.0 mmol) and PPh₃ (18.9 g, 71.9 mmol) in THF (200 mL) was added CBr₄ (23.9 g, 72.0 mmol) in portions at 0° C. under nitrogen atmosphere. The mixture was stirred for 4 h at room temperature. Upon completion, the resulting mixture was diluted with hexane (100 mL). After filtration, the filter cake was washed with hexane (2×100 mL). The combined filtrate was concentrated under reduced pressure. The residue was purified by silica gel column chromatography, eluted with EtOAc:Petroleum ether (v:v=1:5) to afford [[3-(2-bromoethoxy)cyclobutoxy]methyl]benzene (10.1 g, 98%) as a colorless oil. ¹H NMR (400 MHz, DMSO-d₆) δ 7.39-7.24 (m, 5H), 4.36 (s, 2H), 3.71-3.52 (m, 6H), 2.65-2.55 (m, 2H), 1.82-1.75 (m, 2H). LC/MS (ESI, m/z): [(M+18)]⁺=302.05, 304.05.

The following intermediates in Table 30 were synthesized according to the above procedure:

TABLE 30

| Inter- | | | |
|---|---|---|---|
| mediate | Structure | Name | ¹H NMR |
| 133-1 | 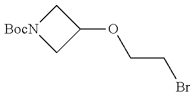 [SMILES: CC(C)(C)OC(=O)N1CC(OCCBr)C1] | tert-butyl 3-(2- bromoethoxy)azeti- dine-1- carboxylate | (400 MHz, CDCl₃) δ 4.35-4.28 (m, 1H), 4.14-4.08 (m, 2H), 3.91-3.87 (m, 2H), 3.73 (t, J = 6.1 Hz, 2H), 3.47 (t, J = 6.1 Hz, 2H), 1.46 (s, 9H) |

### Step 5: 3-(5-[2-[3-(benzyloxy)cyclobutoxy]ethyl]-3-methyl-2-oxo-1,3-benzodiazol-1-yl)piperidine-2,6-dione

Mixture A: A mixture of dtbbpy (4.00 mg, 0.015 mmol) and 1,2-dimethoxyethane dihydrochloride nickel (3.26 mg, 0.015 mmol) in DME (2.00 mL) was stirred for 3 min at room temperature under nitrogen atmosphere. To a mixture of [[3-(2-bromoethoxy)cyclobutoxy]methyl]benzene (632 mg, 2.22 mmol), 3-(5-bromo-3-methyl-2-oxo-1,3-benzodiazol-1-yl)piperidine-2,6-dione (500 mg, 1.48 mmol), tris(trimethylsilyl)silane (367 mg, 1.48 mmol), Na₂CO₃ (470 mg, 4.44 mmol) and IR[DF(CF₃)PPY]₂(DTBPY)PF₆ (12 mg, 0.015 mmol) in DME (3.00 mL) was added the above mixture A at room temperature under nitrogen atmosphere. The reaction was stirred for 2 h under 34 W blue LEDs at room temperature. The resulting mixture was filtered, the filter cake was washed with DCM (2×10.0 mL). The filtrate was concentrated under reduced pressure. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 35%-60% B in 25 min; Flow rate: 80 mL/min; Detector: UV 220/254 nm; desired fractions were collected at 55% B and concentrated under reduced pressure to afford 3-(5-[2-[3-(benzyloxy)cyclobutoxy]ethyl]-3-methyl-2-oxo-1,3-benzodiazol-1-yl)piperidine-2,6-dione (400 mg, 58%) as a white solid. ¹H NMR (400 MHz, DMSO-d₆) δ 11.08 (s, 1H), 7.40-7.24 (m, 5H), 7.08 (d, J=1.5 Hz, 1H), 7.02 (d, J=8.0 Hz, 1H), 6.95-6.90 (m, 1H), 5.40-5.30 (m, 1H), 4.36 (s, 2H), 3.68-3.56 (m, 2H), 3.51 (t, J=7.1 Hz, 2H), 2.97-2.86 (m, 1H), 2.83 (t, J=7.0 Hz, 2H), 2.77-2.63 (m, 2H), 2.62-2.55 (m, 3H), 2.05-1.95 (m, 1H), 1.81-1.70 (m, 2H). LC/MS (ESI, m/z): [(M+1)]⁺=464.25.

The following intermediates in Table 31 were synthesized according to the above procedure:

TABLE 31

| Inter- | | | | |
|---|---|---|---|---|
| medi- | | | MS: | |
| ate | Structure | Name | [(M + 1)]⁺ | ¹H NMR |
| 134-1 | 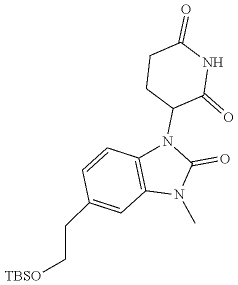 [SMILES: Cn1c(=O)n(C2CCC(=O)NC2=O)c2ccc(CCO[Si](C)(C)C(C)(C)C)cc21] | 3-(5-[2-[(tert- butyldimethylsilyl) oxy]ethyl]-3- methyl-2-oxo-2,3- dihydro-1H-1,3- benzodiazol-1- yl)piperidine-2,6- dione | 418.30 | (400 MHz, DMSO-d₆) δ 11.09 (s, 1H), 7.07 (d, J = 1.5 Hz, 1H), 7.02 (d, J = 8.0 Hz, 1H), 6.95-6.90 (m, 1H), 5.40-5.30 (m, 1H), 3.77 (t, J = 6.9 Hz, 2H), 3.34 (s, 3H), 2.97-2.83 (m, 1H), 2.79 (t, J = 6.9 Hz, 2H), 2.74-2.58 (m, 2H), 2.04-1.96 (m, 1H), 0.86 (s, 9H), 0.01 (s, 6H) |
| 134- 2 | 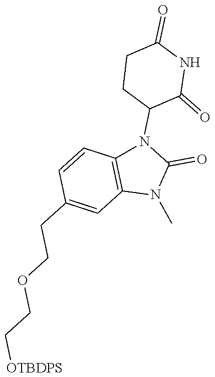 | 3-(5-(2-(2-((tert- butyldiphenylsilyl) oxy)ethoxy)ethyl)- 3-methyl-2-oxo- 2,3-dihydro-1H- benzo[d]imidazol- 1-yl)piperidine- 2,6-dione | 586.20 | (400 MHz, CD₃OD) δ 7.68 (m, 4H), 7.43 (s, 1H), 7.45-7.39 (m, 1H), 7.37-7.30 (m, 4H), 7.05 (d, J = 1.6 Hz, 1H), 7.03-6.94 (m, 2H), 5.35- 5.20 (m, 1H), 3.85-3.75 (m, 2H), 3.73 (t, J = 6.7 Hz, 2H), 3.63-3.56 (m, 2H), 3.33 (s, 3H), 2.93 (d, J = 6.4 Hz, 2H), 2.93-2.85 (m, 1H), 2.84-2.75 (m, 1H), 2.79-2.67 (m, 1H), 2.17-2.08 (m, 1H), 1.02 (s, 9H) |
| 134- 3 | 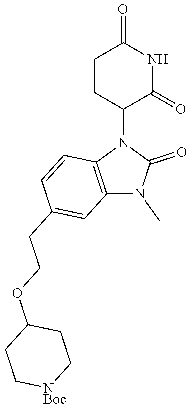 [SMILES: Cn1c(=O)n(C2CCC(=O)NC2=O)c2ccc(CCOC3CCN(C(=O)OC(C)(C)C)CC3)cc21] | tert-butyl 4-[2-[1- (2,6- dioxopiperidin-3- yl)-3-methyl-2- oxo-1,3- benzodiazol-5- yl]ethoxy]piperidine- 1-carboxylate | 487.30 | (400 MHz, DMSO-d₆) δ 7.10 (d, J = 1.6 Hz, 1H), 7.02 (d, J = 8.0 Hz, 1H), 6.95-6.90 (m, 1H), 5.40-5.30 (m, 1H), 3.63-3.56 (m, 4H), 3.50- 3.40 (m, 1H), 3.34 (s, 3H), 3.02 (s, 1H), 2.97-2.80 (m, 2H), 2.78-2.57 (m, 2H), 2.15-2.06 (m, 2H), 2.05- 1.97 (m, 1H), 1.77 (d, J = 12.8 Hz, 2H), 1.39 (s, 9H), 1.32 (m, 2H) |
| 134- 4 | 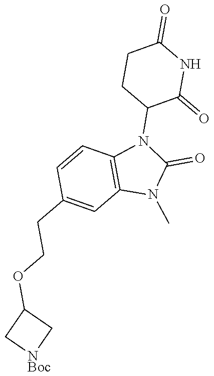 [SMILES: Cn1c(=O)n(C2CCC(=O)NC2=O)c2ccc(CCOC3CN(C(=O)OC(C)(C)C)C3)cc21] | tert-butyl 3-[2-[1- (2,6- dioxopiperidin-3- yl)-3-methyl-2- oxo-1,3- benzodiazol-5- yl]ethoxy]azetidine- 1-carboxylate | 459.10 | (400 MHz, CDCl₃) δ 8.21 (s, 1H), 6.98-6.89 (m, 2H), 6.75 (d, J = 8.0 Hz, 1H), 5.30-5.16 (m, 1H), 4.29- 4.16 (m, 1H), 4.10-4.00 (m, 2H), 3.90-3.75 (m, 2H), 3.59 (t, J = 6.9 Hz, 2H), 3.45 (s, 3H), 3.00-2.90 (m, 3H), 2.85-2.75 (m, 2H), 2.29-2.21 (m, 1H), 1.45 (s, 9H) |
| 134- 5 | 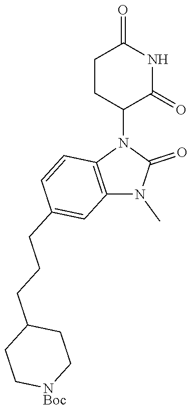 [SMILES: Cn1c(=O)n(C2CCC(=O)NC2=O)c2ccc(CCCC3CCN(C(=O)OC(C)(C)C)CC3)cc21] | tert-butyl 4-[3-[1- (2,6- dioxopiperidine-3- yl)-3-methyl-2- oxo-1,3- benzodiazol-5- yl]propyl]piperidine- 1-carboxylate | 485.25 | (400 MHz, CDCl₃) δ 8.16 (s, 1H), 6.95-6.90 (m, 1H), 6.86 (d, J = 1.5 Hz, 1H), 6.73 (d, J = 8.0 Hz, 1H), 5.30-5.18 (m, 1H), 4.09 (d, J = 13.2 Hz, 2H), 3.45 (s, 3H), 3.01-2.71 (m, 4H), 2.71-2.62 (m, 4H), 2.30-2.21 (m, 1H), 1.70-1.62 (m, 4H), 1.47 (s, 9H), 1.35-1.20 (m, 2H), 1.15-109 (m, 2H) |

### Step 6: 3-[5-[2-(3-hydroxycyclobutoxy)ethyl]-3-methyl-2-oxo-1,3-benzodiazol-1-yl]piperidine-2,6-dione

To a stirred solution of 3-(5-[2-[3-(benzyloxy)cyclobutoxy]ethyl]-3-methyl-2-oxo-1,3-benzodiazol-1-yl)piperidine-2,6-dione (1.80 g, 3.88 mmol) in AcOH (30.0 mL) was added 10% Pd(OH)2 on activated carbon (200 mg) at room temperature under nitrogen atmosphere. The mixture was purged with H₂ three times at room temperature. The mixture was stirred for 16 h at room temperature under hydrogen atmosphere. Upon completion, the resulting mixture was filtered. The filter cake was washed with DCM (3×20.0 mL). The filtrate was concentrated under reduced pressure. The residue was purified by reversed phase flash chromatography with the following conditions: Column: WelFlash™ C18-I, 20-40 um, 330 g; Eluent A: water (plus 10 mmol/L NH₄HCO₃); Eluent B: ACN; Gradient: 20%-40% B in 20 min; Flow rate: 80 mL/min; Detector: UV 220/254 nm; desired fractions were collected at 34% B and concentrated under reduced pressure to afford 3-[5-[2-(3-hydroxycyclobutoxy)ethyl ]-3-methyl-2-oxo-1,3-benzodiazol-1-yl]piperidine-2,6-dione (1.15 g, 79%) as a white solid. ¹H NMR (400 MHz, DMSO-d₆) δ 7.07 (d, J=1.6 Hz, 1H), 7.01 (d, J=8.1 Hz, 1H), 6.95-6.87 (m, 1H), 5.40-5.30 (m, 1H), 4.97 (d, J=6.7 Hz, 1H), 3.72-3.60 (m, 1H), 3.48 (t, J=7.0 Hz, 3H), 3.32 (s, 3H), 2.97-2.85 (m, 1H), 2.81 (t, J=7.1 Hz, 2H), 2.76-2.67 (m, 1H), 2.65-2.60 (m, 1H), 2.55 (d, J=3.0 Hz, 2H), 2.04-1.97 (m, 1H), 1.62-1.56 (m, 2H). LC/MS (ESI, m/z): [(M+1)]⁺=374.15

### INTERMEDIATE T4: 3-[5-(2-hydroxyethyl)-3-methyl-2-oxo-2,3-dihydro-1H-1,3-benzodiazol-1-yl]piperidine-2,6-dione

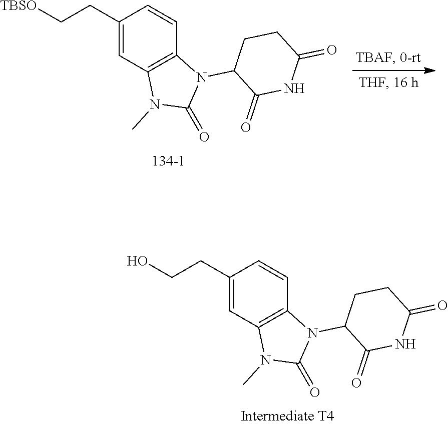 [SMILES: Cn1c(=O)n(C2CCC(=O)NC2=O)c2ccc(CCO[Si](C)(C)C(C)(C)C)cc21 ; Cn1c(=O)n(C2CCC(=O)NC2=O)c2ccc(CCO)cc21]

To a stirred solution of 3-(5-[2-[(tert-butyldimethylsilyl)oxy]ethyl]-3-methyl-2-oxo-2,3-dihydro-1H-1,3-benzodiazol-1-yl)piperidine-2,6-dione (540 mg, 1.29 mmol) in THF (20.0 mL) was added TBAF (676 mg, 2.59 mmol) in portions at 0° C. The resulting mixture was stirred for 16 h at room temperature under nitrogen atmosphere. The resulting mixture was concentrated under reduced pressure. The residue was purified by reversed phase flash chromatography with the following conditions: Column: WelFlash™ C18-I, 20-40 um, 330 g; Eluent A: water (plus 10 mmol/L AcOH); Eluent B: ACN; Gradient: 15%-35% B in 25 min; Flow rate: 80 mL/min; Detector: UV 220/254 nm; desired fractions were collected at 23% B and concentrated under reduced pressure to afford 3-[5-(2-hydroxyethyl)-3-methyl-2-oxo-2,3-dihydro-1H-1,3-benzodiazol-1-yl]piperidine-2,6-dione (370 mg, 94%) as a white solid. ¹H NMR (400 MHz, DMSO-d₆) δ 11.09 (s, 1H), 7.09-6.96 (m, 2H), 6.91-6.87 (m, 1H), 5.40-5.30 (m, 1H), 4.63 (t, J=5.2 Hz, 1H), 3.61 (td, J=7.0, 5.1 Hz, 2H), 3.33 (s, 3H), 2.95-2.90 (m, 1H), 2.80-2.57 (m, 4H), 2.06-1.97 (m, 1H). LC/MS (ESI, m/z): [(M+1)]⁺=304.25

The following intermediates in Table 32 were synthesized according to the above procedure:

TABLE 32

| Inter- | | | | |
|---|---|---|---|---|
| medi- | | | MS: | |
| ate | Structure | Name | [(M + 1)]⁺ | ¹H NMR |
| T5 | 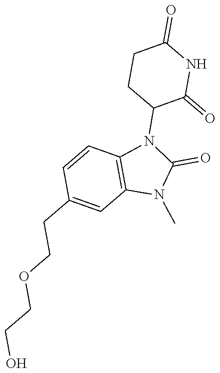 [SMILES: Cn1c(=O)n(C2CCC(=O)NC2=O)c2ccc(CCOCCO)cc21] | 3-[5-[2-(2- hydroxyethoxy)eth- yl]-3-methyl- 2-oxo-2,3- dihydro-1H-1,3- benzodiazol-1- yl]piperidine- 2,6-dione | 348.05 | (400 MHz, DMSO-d₆) δ 11.10 (s, 1H), 7.11 (d, J = 1.5 Hz, 1H), 7.01 (d, J = 8.1 Hz, 1H), 6.95-6.90 (m, 1H), 5.40- 5.30 (m, 1H), 4.60 (t, J = 5.5 Hz, 1H), 3.62 (t, J = 7.1 Hz, 2H), 3.55-3.45 (m, 2H), 3.44-3.38 (m, 2H), 3.34 (s, 3H), 2.97-2.81 (m, 3H), 2.78-2.62 (m, 2H), 2.08 (s, 1H) |

### INTERMEDIATE T7: 3-[4-[(3-hydroxypropoxy)methyl]-3-methyl-2-oxo-1,3-benzodiazol-1-yl]piperidine-2,6-dione

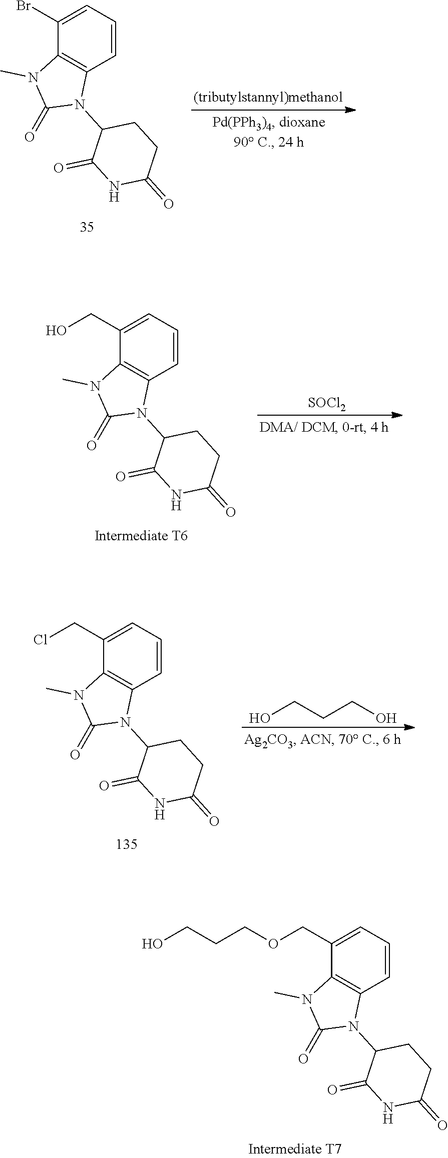 [SMILES: Cn1c(=O)n(C2CCC(=O)NC2=O)c2cccc(Br)c21 ; Cn1c(=O)n(C2CCC(=O)NC2=O)c2cccc(CO)c21 ; "SOCl2" ; Cn1c(=O)n(C2CCC(=O)NC2=O)c2cccc(CCl)c21 ; OCCCO ; Cn1c(=O)n(C2CCC(=O)NC2=O)c2cccc(COCCCO)c21]

### Step 1: 3-[4-(hydroxymethyl)-3-methyl-2-oxo-1,3-benzodiazol-1-yl]piperidine-2,6-dione

To a stirred mixture of 3-(4-bromo-3-methyl-2-oxo-1,3-benzodiazol-1-yl)piperidine-2,6-dione (500 mg, 1.48 mmol) and Pd(PPh₃)₄ (171 mg, 0.15 mmol) in 1,4-dioxane (10.0 mL) was added (tributylstannyl)methanol (950 mg, 2.96 mmol) at room temperature under nitrogen atmosphere. The mixture was stirred for 24 h at 90° C. The mixture was cooled down to room temperature. The reaction was quenched with KF aq. (10.0 mL) at room temperature. The resulting mixture was extracted with EtOAc (3×20.0 mL). The combined organic layers were dried over anhydrous Na₂SO₄. After filtration, the filtrate was concentrated under reduced pressure. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 20%-40% B in 20 min; Flow rate: 80 mL/min; Detector: UV 220/254 nm; desired fractions were collected at 30% B and concentrated under reduced pressure to afford 3-[4-(hydroxymethyl)-3-methyl-2-oxo-1,3-benzodiazol-1-yl]piperidine-2,6-dione (200 mg, 47%) as white solid. ¹H NMR (400 MHz, DMSO-d₆) δ 11.10 (s, 1H), 7.10-7.05 (m, 1H), 7.05-7.01 (m, 1H), 7.01-6.95 (m, 1H), 5.45-5.35 (m, 2H), 4.75 (s, 2H), 3.62 (s, 3H), 2.95-2.85 (m, 1H), 2.80-2.70 (m, 1H), 2.69-2.59 (m, 1H), 2.06-1.96 (m, 1H). LC/MS (ESI, m/z): [(M+23)]⁺=290.25

### Step 2: 3-[4-(chloromethyl)-3-methyl-2-oxo-1,3-benzodiazol-1-yl]piperidine-2,6-dione

To a stirred solution of 3-[4-(hydroxymethyl)-3-methyl-2-oxo-1,3-benzodiazol-1-yl]piperidine-2,6-dione (3.50 g, 12.1 mmol) in DCM (20.0 mL) and DMA (10.0 mL) was added SOCl₂ (1.76 mL, 14.8) at 0° C. under nitrogen atmosphere and stirred at room temperature for 4 h. The reaction was quenched by the addition of water (200 mL) at 0° C. The resulting mixture was extracted with CH₂Cl₂ (300 mL). The combined organic layers were washed with brine (100 mL) and dried over anhydrous Na₂SO₄. After filtration, the filtrate was concentrated under reduced pressure to afford 3-[4-(chloromethyl)-3-methyl-2-oxo-1,3-benzodiazol-1-yl]piperidine-2,6-dione (3.50 g, 94%) as an orange solid. ¹H NMR (400 MHz, DMSO-d₆) δ 11.11 (d, J=10.5 Hz, 1H), 7.10-6.95 (m, 3H), 5.45-5.35 (m, 1H), 4.75(s, 2H), 3.62 (s, 3H), 2.95-2.85 (m, 1H), 2.80-2.70 (m, 1H), 2.69-2.58 (m, 1H), 2.05-1.97 (m, 1H). LC/MS (ESI, m/z): [(M+23)]⁺=308.15, 310.15

### Step 3: 3-[4-[(3-hydroxypropoxy)methyl]-3-methyl-2-oxo-1,3-benzodiazol-1-yl]piperidine-2,6-dione

To a stirred mixture of 1,3-propandiol (866 mg, 11.37 mmol) and Ag₂CO₃ (941 mg, 3.41 mmol) in ACN (10.0 mL)/DMA (2.00 mL) was added 3-[4-(chloromethyl)-3-methyl-2-oxo-1,3-benzodiazol-1-yl]piperidine-2,6-dione (350 mg, 1.14 mmol) at room temperature under nitrogen atmosphere. The mixture was stirred for 6 h at 70° C. Upon completion, the resulting mixture was filtered, the filter cake was washed with DCM (3×10.0 mL). The filtrate was concentrated under reduced pressure. The residue was purified by reversed phase flash chromatography with the following conditions: Column: WelFlash™ C18-I, 20-40 um, 80 g; Eluent A: water (plus 10 mmol/L FA); Eluent B: ACN; Gradient: 15%-35% B in 20 min; Flow rate: 50 mL/min; Detector: UV 220/254 nm; desired fractions were collected at 25% B and concentrated under reduced pressure to afford 3-[4-[(3-hydroxypropoxy)methyl]-3-methyl-2-oxo-1,3-benzodiazol-1-yl]piperidine-2,6-dione (230 mg, 58%) as a white solid. ¹H NMR (400 MHz, DMSO-d₆) δ 11.10 (s, 1H), 7.15-7.05 (m, 1H), 7.06-6.96 (m, 2H), 5.45-5.35 (m, 1H), 4.68 (s, 2H), 4.40 (t, J=5.1 Hz, 1H), 3.58 (s, 3H), 3.55 (t, J=6.5 Hz, 2H), 3.50-3.40 (m, 2H), 2.97-2.84 (m, 1H), 2.80-2.59 (m, 2H), 2.07-1.99 (m, 1H), 1.75-1.60 (m, 2H). LC/MS (ESI, m/z): [(M+23)]⁺=370.25.

The following intermediates in Table 33 were synthesized according to the above procedure:

TABLE 33

| Inter- | | | | |
|---|---|---|---|---|
| medi- | | | MS: | |
| ate | Structure | Name | [(M + )]⁺ | ¹H NMR |
| T8 | 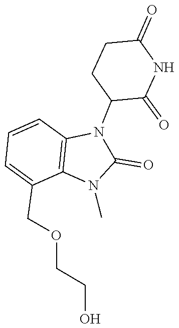 [SMILES: Cn1c(=O)n(C2CCC(=O)NC2=O)c2cccc(COCCO)c21] | 3-[4-[(2- hydroxyethoxy) methyl]-3- methyl-2-oxo- 1,3-benzodiazol- 1-yl]piperidine- 2,6-dione | 334.15 | (400 MHz, DMSO-d₆) δ 11.10 (s, 1H), 7.12 (d, J = 7.5 Hz, 1H), 7.07-6.95 (m, 2H), 5.45-5.35 (m, 1H), 4.72 (s, 2H), 4.67 (t, J = 5.3 Hz, 1H), 3.60 (s, 3H), 3.54 (h, J = 4.6 Hz, 4H), 2.95-2.82 (m, 1H), 2.80-2.59 (m, 2H), 2.02 (t, J = 5.4 Hz, 1H) |
| T9 | 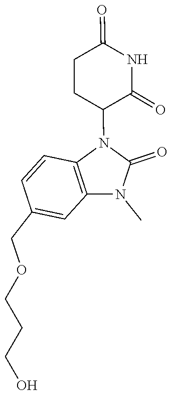 [SMILES: Cn1c(=O)n(C2CCC(=O)NC2=O)c2ccc(COCCCO)cc21] | 3-[5-[(3- hydroxypropoxy) methyl]-3- methyl-2-oxo- 1,3-benzodiazol- 1-yl]piperidine- 2,6-dione | 348.20 | (400 MHz, DMSO-d₆) δ 11.06 (s, 1H), 7.15 (s, 1H), 7.09 (d, J = 8.0 Hz, 1H), 7.01 (d, J = 8.1 Hz, 1H), 5.45-5.34 (m, 1H), 4.47 (s, 2H), 4.40 (s, 1H), 3.53- 3.44 (m, 4H), 3.35 (s, 3H), 2.98-2.84 (m, 1H), 2.78-2.58 (m, 2H), 2.10-1.98 (m, 1H), 1.75-1.60 (m, 2H) |
| T10 | 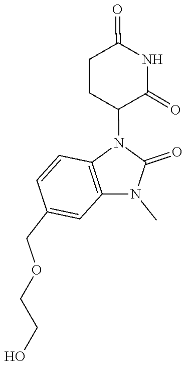 [SMILES: Cn1c(=O)n(C2CCC(=O)NC2=O)c2ccc(COCCO)cc21] | 3-[5-[(3- hydroxypropoxy) methyl]-3- methyl-2-oxo- 1,3-benzodiazol- 1-yl]piperidine- 2,6-dione | 334.15 | (400 MHz, DMSO-d₆) δ 11.09 (s, 1H), 7.18 (d, J = 1.5 Hz, 1H), 7.09 (d, J = 8.0 Hz, 1H), 7.07-7.00 (m, 1H), 5.45- 5.33 (m, 1H), 4.61 (t, J = 5.5 Hz, 1H), 4.51 (s, 2H), 3.60-3.50 (m, 2H), 3.47 (d, J = 5.1 Hz, 2H), 3.35 (s, 3H), 3.00- 2.88 (m, 1H), 2.80-2.66 (m, 1H), 2.66- 2.59 (m, 1H), 2.06-1.98 (m, 1H) |

### INTERMEDIATE T11: 3-(5-[[4-(3-hydroxypropoxy)piperidin-1-yl]methyl]-3-methyl-2-oxo-1,3-benzodiazol-1-yl)piperidine-2,6-dione

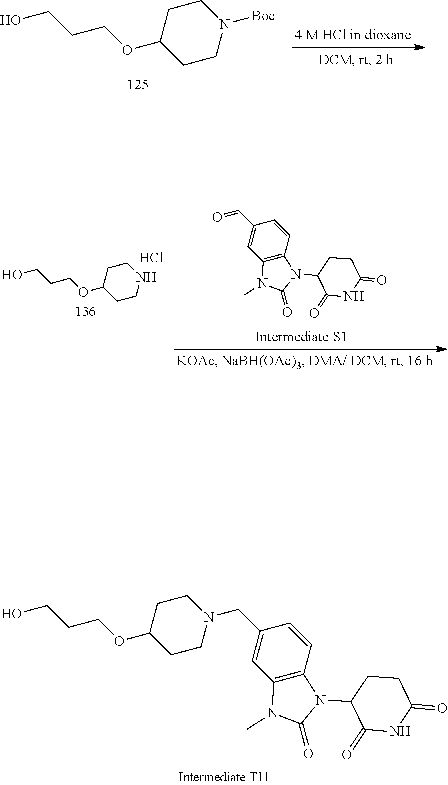 [SMILES: CC(C)(C)OC(=O)N1CCC(OCCCO)CC1 ; OCCCOC1CCNCC1 ; Cl ; Cn1c(=O)n(C2CCC(=O)NC2=O)c2ccc(C=O)cc21 ; Cn1c(=O)n(C2CCC(=O)NC2=O)c2ccc(CN3CCC(OCCCO)CC3)cc21]

### Step 1: 3-(piperidin-4-yloxy)propan-1-ol hydrochloride

To a stirred solution of tert-butyl 4-(3-hydroxypropoxy)piperidine-1-carboxylate (2.00 g, 7.71 mmol) in DCM (10.0 mL) was added HCl (4 M in 1,4-dioxane, 10.0 mL) at room temperature and stirred for 2 h at room temperature. The resulting mixture was concentrated under reduced pressure to afford 3-(piperidin-4-yloxy)propan-1-ol hydrochloride (1.60 g, crude) as a yellow oil. ¹H NMR (400 MHz, DMSO-d₆) δ 8.93 (s, 1H), 3.58-3.51 (m, 1H), 3.50-3.40 (m, 4H), 3.10 (s, 2H), 3.00-2.85 (m, 2H), 2.00-1.90 (m, 2H), 1.72-1.57 (m, 4H).

The following intermediates in Table 34 were synthesized according to the above procedure:

TABLE 34

| Inter- | | | MS: | |
|---|---|---|---|---|
| mediate | Structure | Name | [(M + 1)]⁺ | ¹H NMR |
| 126-1 | 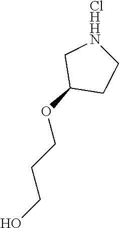 | 3-[(3R)-pyrrolidin-3- yloxy]propan-1-ol hydrochloride | — | (400 MHz, DMSO-d₆) δ 9.32 (br, 2H [symbol] 4.27 (s, 1H), 4.20- 4.05 (m, 1H), 3.57 (s [symbol] 2H), 3.50-3.40 (m, 2H), 3.22-3.15 ( [symbol] 4H), 2.05-1.90 (m, 1H), 1.95-1.83 ( [symbol] 1H), 1.70-1.50 (m, 2H) |
| 126-2 | 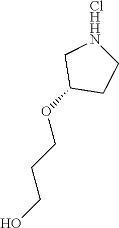 | 3-[(3S)-pyrrolidin-3- yloxy]propan-1-ol hydrochloride | — | (400 MHz, CDCl3) δ 4.26 (d, J = 4.5 [symbol] 1H), 3.70-3.50 (m, 4H), 3.43-3.22 [symbol] 4H), 2.26- 2.15 (m, 1H), 2.10-2.00 [symbol] 1H), 1.90-1.72 (m, 2H) |
| 126-3 | 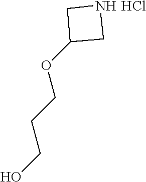 | 3-(azetidin-3-yloxy)- propan-1-ol hydro- chloride | 132.10 | — |

[symbol]

indicates data missing or illegible when filed

### Step 2: 3-(5-[[4-(3-hydroxypropoxy)piperidin-1-yl]methyl]-3-methyl-2-oxo-1,3-benzodiazol-1-yl)piperidine-2,6-dione

To a stirred solution of 1-(2,6-dioxopiperidin-3-yl)-3-methyl-2-oxo-1,3-benzodiazole-5-carbaldehyde (900 mg, 3.13 mmol) and 3-(piperidin-4-yloxy)propan-1-ol hydrochloride (1.23 g, 6.26 mmol) in DCM (20.0 mL) was added KOAc (1.23 g, 12.5 mmol) at room temperature under nitrogen atmosphere. Then NaBH(OAc)₃ (1.99 g, 9.40 mmol) was added to the reaction. The reaction was reacted for 1 h at room temperature. Upon completion, the solution was concentrated under reduced pressure. The residue was purified by reversed phase flash chromatography with the following conditions: WelFlash™ C18-I, 20-40 um, 330 g; Eluent A: water (plus 10 mmol/L NH₄HCO₃); Eluent B: ACN; Gradient: 10%-15% B in 20 min; Flow rate: 80 mL/min; Detector: UV 220/254 nm; desired fractions were collected at 14% B and concentrated under reduced pressure to afford 3-(5-[[4-(3-hydroxypropoxy)piperidin-1-yl]methyl]-3-methyl -2-oxo-1,3-benzodiazol-1-yl)piperidine-2,6-dione (700 mg, 52%)as a white solid. ¹H NMR (400 MHz, DMSO-d₆) δ 11.09 (s, 1H), 7.10 (d, J=1.5 Hz, 1H), 7.04 (d, J=8.0 Hz, 1H), 7.00-6.90 (m, 1H), 5.40-5.30 (m, 1H), 4.34 (t, J=5.0 Hz, 1H), 3.43 (t, J=6.4 Hz, 6H), 3.34 (s, 3H), 3.30-3.20 (m, 1H), 2.97-2.84 (m, 1H), 2.78-2.66 (m, 2H), 2.66-2.64 (m, 1H), 2.64-2.57 (m, 1H), 2.12-1.96 (m, 3H), 1.85-1.70 (m, 2H), 1.62 (p, J=6.4 Hz, 2H), 1.48-1.35 (m, 2H). LC/MS (ESI, m/z): [(M+23)]⁺=431.20.

The following intermediates in Table 35 were synthesized according to the above procedure:

TABLE 35

| Inter- | | | | |
|---|---|---|---|---|
| medi- | | | MS: | |
| ate | Structure | Name | [(M + 1)]⁺ | ¹H NMR |
| T12 | 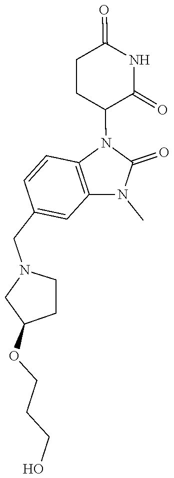 [SMILES: Cn1c(=O)n(C2CCC(=O)NC2=O)c2ccc(CN3CC[C@@H](OCCCO)C3)cc21] | 3-(5-[[(3R)-3- (3- hydroxypropoxy) pyrrolidin-1- yl]methyl]-3- methyl-2-oxo- 1,3- benzodiazol-1- yl)piperidine- 2,6-dione | 417.20 | (400 MHz, DMSO-d₆) δ 7.11 (d, J = 1.5 Hz, 1H), 7.04 (d, J = 8.0 Hz, 1H), 7.00-6.90 (m, 1H), 5.40-5.30 (m, 1H), 4.36 (s, 1H), 4.00-3.91 (m, 1H), 3.57 (s, 2H), 3.43 (t, J = 6.3 Hz, 2H), 3.38 (d, J = 2.7 Hz, 5H), 2.97-2.84 (m, 1H), 2.83-2.60 (m, 3H), 2.60-2.53 (m, 1H), 2.42 (td, J = 9.1, 4.8 Hz, 2H), 2.00 (m, 2H), 1.65-1.50 (m, 3H). |
| T13 | 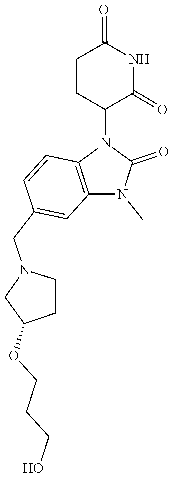 [SMILES: Cn1c(=O)n(C2CCC(=O)NC2=O)c2ccc(CN3CC[C@H](OCCCO)C3)cc21] | 3-(5-[[(3S)-3- (3- hydroxypropoxy) pyrrolidin-1- yl]methyl]-3- methyl-2-oxo- 1,3- benzodiazol-1- yl)piperidine- 2,6-dione | 417.20 | (400 MHz, DMSO-d₆) δ 7.11 (d, J = 1.4 Hz, 1H), 7.04 (d, J = 8.0 Hz, 1H), 7.00-6.90 (m, 1H), 5.40-5.30 (m, 1H), 4.35 (s, 1H), 4.00-3.90 (m, 1H), 3.58 (s, 2H), 3.43 (t, J = 6.3 Hz, 2H), 3.39- 3.28 (m, 5H), 2.97-2.84 (m, 1H), 2.81- 2.59 (m, 3H), 2.59-2.52 (m, 1H), 2.48- 2.35 (m, 2H), 2.10-1.95 (m, 2H), 1.65- 1.50 (m, 3H) |
| T14 | 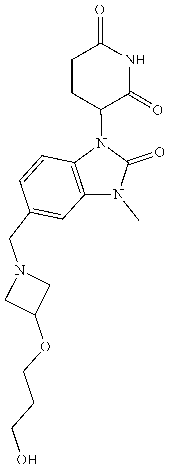 [SMILES: Cn1c(=O)n(C2CCC(=O)NC2=O)c2ccc(CN3CC(OCCCO)C3)cc21] | 3-(5-[[3-(3- hydroxypropoxy) azetidin-1- yl]methyl]-3- methyl-2-oxo- 1,3- benzodiazol-1- yl)piperidine- 2,6-dione | 403.10 | (400 MHz, DMSO-d₆) δ 11.08 (s, 1H), 7.09 (d, J = 1.5 Hz, 1H), 7.03 (d, J = 8.0 Hz, 1H), 6.99-6.91 (m, 1H), 5.40- 5.30 (m, 1H), 4.39 (t, J = 5.1 Hz, 1H), 4.03 (p, J = 5.9 Hz, 1H), 3.58 (s, 2H), 3.49-3.41 (m, 4H), 3.38-3.33 (m, 5H), 2.97-2.86 (m, 1H), 2.90-2.80 (m, 2H), 2.77-2.69 (m, 1H), 2.67-2.58 (m, 1H), 2.05-1.97 (m, 1H), 1.65-1.50 (m, 2H) |

### INTERMEDIATE T15: tert-butyl N-[9-(2,6-dioxopiperidin-3-yl)pyrido[2,3-b]indol-6-yl]-N-[2-[2-(2-hydroxyethoxy)ethoxy]ethyl]carbamate

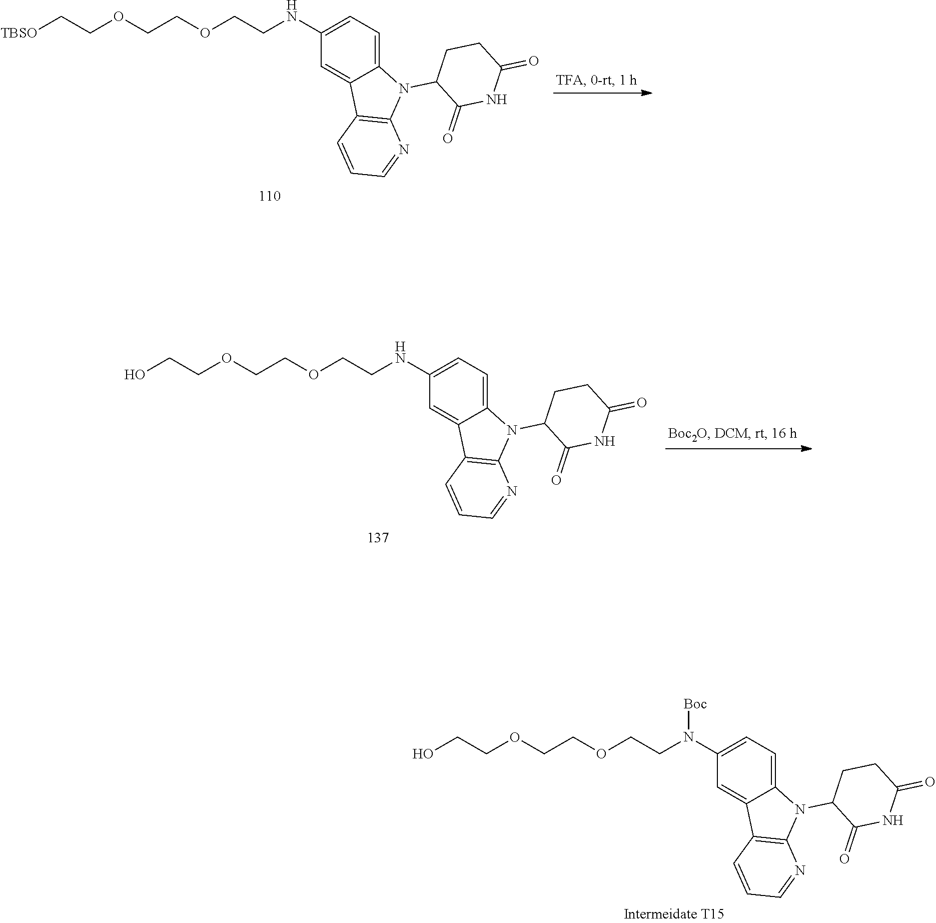 [SMILES: CC(C)(C)[Si](C)(C)OCCOCCOCCNc1ccc2c(c1)c1cccnc1n2C1CCC(=O)NC1=O ; O=C1CCC(n2c3ccc(NCCOCCOCCO)cc3c3cccnc32)C(=O)N1 ; CC(C)(C)OC(=O)N(CCOCCOCCO)c1ccc2c(c1)c1cccnc1n2C1CCC(=O)NC1=O]

### Step 1: 3-[6-([2-[2-(2-hydroxyethoxy)ethoxy]ethyl]amino)pyrido[2,3-b]indol-9-yl]piperidine-2,6-dione

To a stirred solution of 3-[6-(11,11,12,12-tetramethyl-4,7,10-trioxa-1-aza-11-silatridecan-1-yl)pyrido[2,3-b]indol-9-yl]piperidine-2,6-dione (456 mg, 0.84 mmol) in DCM (10.0 mL) was added trifluoroacetic acid (1.00 mL) at 0° C. under nitrogen atmosphere. The reaction was stirred for 1 h at room temperature. The resulting mixture was concentrated under vacuum and the residue was purified by reversed phase flash chromatography with the following conditions: column WelFlash™ C18-I, 20-40 um, 330 g; Eluent A: water (plus 10 mmol/L FA); Eluent B: ACN; Gradient: 25%-45% B in 20 min; Flow rate: 80 mL/min; Detector: UV 220/254 nm; desired fractions were collected at 34% B and concentrated under reduced pressure to afford 3-[6-([2-[2-(2-hydroxyethoxy)ethoxy]ethyl]amino)pyrido[2,3-b]indol-9-yl]piperidine-2,6-dione (310 mg, 86%) as a white solid. ¹H NMR (400 MHz, DMSO-d₆) δ 11.11 (s, 1H), 8.46-8.37 (m, 1H), 8.35-8.30 (m, 1H), 8.05 (s, 1H), 7.40-7.26 (m, 2H), 7.20-7.10 (m, 1H), 5.91 (s, 1H), 5.33 (t, J=5.9 Hz, 1H), 3.64-3.38 (m, 12H), 3.03-2.96 (m, 3H), 2.07 (s, 1H). LC/MS (ESI, m/z): [(M+23)]⁺=427.30.

### Step 2: tert-butyl N-[9-(2,6-dioxopiperidin-3-yl)pyrido[2,3-b]indol-6-yl]-N-[2-[2-(2-hydroxyethoxy)ethoxy]ethyl]carbamate

To a stirred solution of 3-[6-([2-[2-(2-hydroxyethoxy)ethoxy]ethyl]amino)pyrido[2,3-b]indol-9-yl]piperidine-2,6-dione (270 mg, 0.63 mmol) in DCM (30.0 mL) was added Boc₂O (207 mg, 0.95 mmol) at room temperature under nitrogen atmosphere. The reaction was stirred for 16 h at room temperature. Upon completion, the solution was concentrated under reduced pressure. The residue was purified by reversed phase flash chromatography with the following conditions: column WelFlash™ C18-I, 20-40 um, 330 g; Eluent A: water (plus 10 mmol/L FA); Eluent B: ACN; Gradient: 45%-65% B in 20 min; Flow rate: 80 mL/min; Detector: UV 220/254 nm; desired fractions were collected at 54% B and concentrated under reduced pressure to afford tert-butyl N-[9-(2,6-dioxopiperidin-3-yl)pyrido[2,3-b]indol-6-yl]-N-[2-[2-(2-hydroxyethoxy)ethoxy]ethyl]carbamate (320 mg, 96%). ¹H NMR (400 MHz, DMSO-d₆) δ 11.18 (s, 1H), 8.60-8.50 (m, 1H), 8.49-8.37 (m, 1H), 8.14 (d, J=2.1 Hz, 1H), 7.59 (d, J=8.4 Hz, 1H), 7.45-7.35 (m, 1H), 7.30-7.20 (m, 1H), 6.04 (s, 1H), 4.57 (s, 1H), 3.79 (t, J=6.0 Hz, 2H), 3.58-3.48 (m, 6H), 3.47 (d, J=4.6 Hz, 2H), 3.10-2.90 (m, 2H), 2.76-2.63 (m, 3H), 2.18-2.00 (m, 1H), 1.59-1.19 (m, 9H). LC/MS (ESI, m/z): [(M+23)]⁺=527.30.

### INTERMEDIATE 138: 2-[3-(prop-2-yn-1-yloxy)propoxy]ethan-1-ol

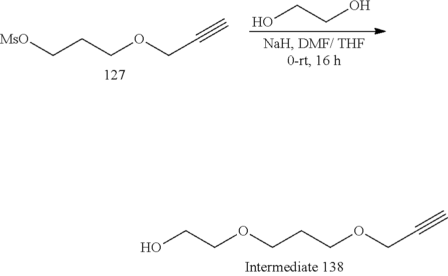

To a stirred solution of ethane-1,2-diol (25.8 g, 416 mmol) in THF (1.00 L) and DMF (100 mL) was added NaH (4.99 g, 208 mmol, 60% dispersion in mineral oil) in portions over 30 min at 0° C. under nitrogen atmosphere followed by the addition of a solution of 3-(prop-2-yn-1-yloxy)propyl methanesulfonate (20.0 g, 104 mmol) in THF (20.0 mL) over 5 min. The resulting mixture was stirred for 16 h at room temperature under nitrogen atmosphere. The resulting mixture was quenched with water (2.00 L) and extracted with CH₂Cl₂ (2×500 mL). The combined organic phase was dried over anhydrous Na₂SO₄. After filtration, the filtrate was concentrated under reduced pressure. The residue was purified by silica gel column chromatography, eluted with Petroleum ether/EtOAc (v:v=5:1) to afford 2-[3-(prop-2-yn-1-yloxy)propoxy]ethan-1-ol (8.50 g, 52%) as a yellow oil. ¹H NMR (400 MHz, DMSO-d₆) δ 4.57 (t, J=5.5 Hz, 1H), 4.11 (d, J=2.4 Hz, 2H), 3.55-3.45 (m, 4H), 3.44-3.29 (m, 5H), 1.74 (p, J=6.5 Hz, 2H)

### INTERMEDIATE S2: 4-[3-[1-(2,6-dioxopiperidin-3-yl)-3-methyl-2-oxo-2,3-dihydro-1H-1,3-benzodiazol-5-yl]propoxy]butanal

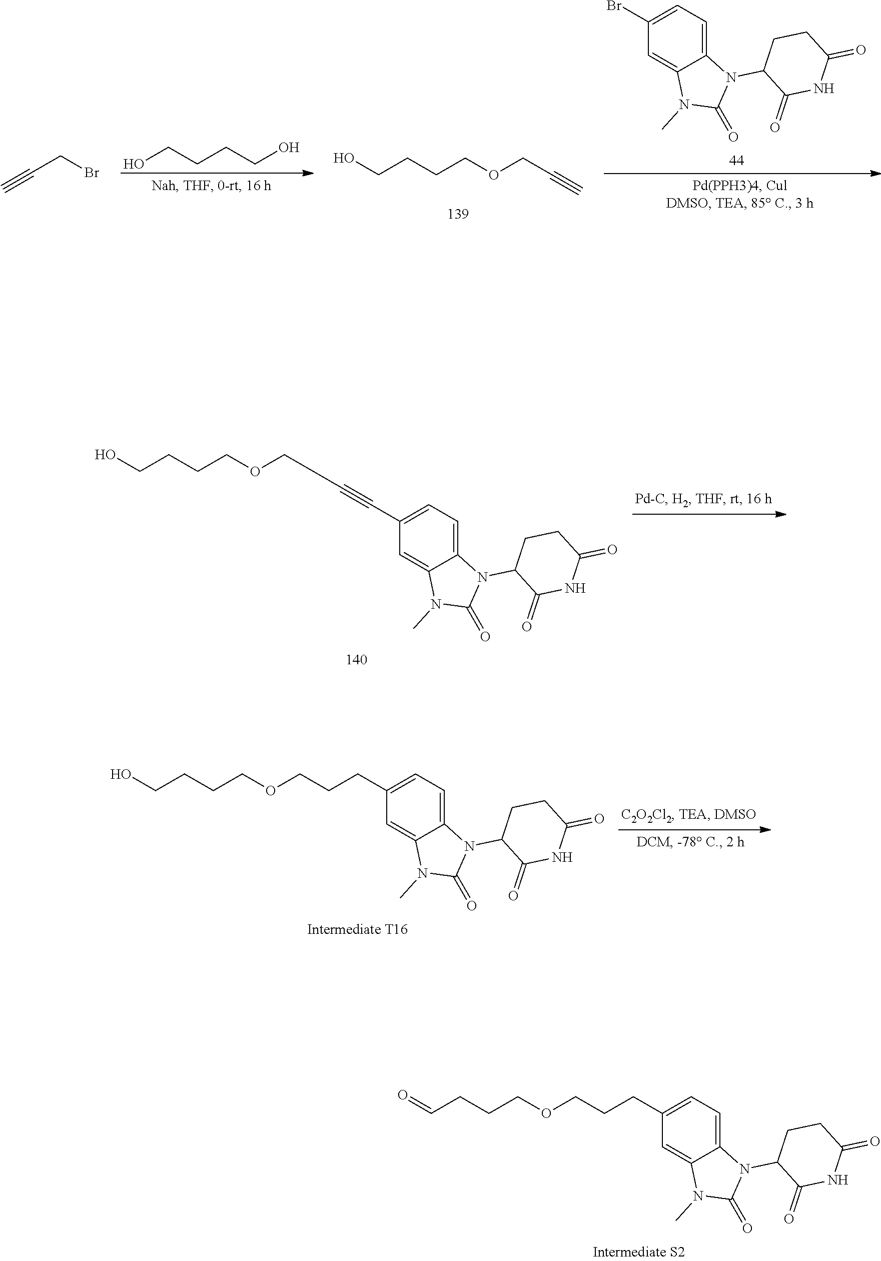

### Step 1: 4-(prop-2-yn-1-yloxy)butan-1-ol

To a stirred solution of butane-1,4-diol (190 g, 2.10 mol) in THF (2.50 L) was added NaH (20.2 g, 841 mmol, 60% dispersion in mineral oil) in portions over 5 min at 0° C. under nitrogen atmosphere. Followed by the addition of 3-bromoprop-1-yne (50.0 g, 420 mmol) over 5 min. The resulting mixture was stirred for 16 h at room temperature under nitrogen atmosphere. The reaction was quenched with sat. NH₄Cl (500 mL) at 0° C. and extracted with EtOAc (3×1.00 L). The combined organic layers was washed with brine (1.00 L) and dried over anhydrous Na₂SO₄. After filtration, the filtrate was concentrated under reduced pressure. The residue was purified by silica gel column chromatography, eluted with 10%˜30% EtOAc in petroleum ether. The fractions containing desired product were collected and concentrated under reduced pressure to afford 4-(prop-2-yn-1-yloxy)butan-1-ol (9.40 g, 18%) as a light yellow oil. ¹HNMR (400 MHz, CDCl₃) δ 4.17 (d, J=2.4 Hz, 2H), 3.69 (t, J=5.8 Hz, 2H), 3.59 (t, J=5.8 Hz, 2H), 2.50-2.40 (m, 1H), 1.78-1.65 (m, 4H)

### Step 2: 3-[5-[3-(4-hydroxybutoxy)prop-1-yn-1-yl]-3-methyl-2-oxo-2,3-dihydro-1H-1,3-benzodiazol-1-yl]piperidine-2,6-dione

To a stirred mixture of 4-(prop-2-yn-1-yloxy)butan-1-ol (1.90 g, 14.8 mmol) in DMSO (30.0 mL) was added Pd(PPh₃)₄ (342 mg, 0.30 mmol) and CuI (56 mg, 0.30 mmol) at 25° C. under nitrogen atmosphere. To the above mixture was added 3-(5-bromo-3-methyl-2-oxo-1,3-benzodiazol-1-yl)piperidine-2,6-dione (1.00 g, 2.96 mmol) and TEA (10.0 mL) at 25° C. The resulting mixture was stirred for additional 3 h at 85° C. under nitrogen atmosphere. The resulting mixture was cooled and concentrated under vacuum. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 20%-40% B in 25 min; Flow rate: 80 mL/min; Detector: UV 254/220 nm; desired fractions were collected at 28% B and concentrated under reduced pressure to afford 3-[5-[3-(4-hydroxybutoxy)prop-1-yn-1-yl]-3-methyl-2-oxo-1,3-benzodiazol-1-yl]piperidine-2,6-dione (670 mg, 59%) as a white solid. ¹H NMR (400 MHz, DMSO-d₆) δ 11.12 (s, 1H), 7.34 (d, J=1.4 Hz, 1H), 7.19-7.12 (m, 2H), 5.50-5.30 (m, 1H), 4.40-4.34 (m, 4H), 3.52 (t, J=6.4 Hz, 2H), 3.42 (s, 3H), 2.95-2.84 (m, 1H), 2.79-2.57 (m, 2H), 2.09-1.98 (m, 1H), 1.63-1.39 (m, 4H). LC/MS (ESI, m/z): [(M+1)]⁺=386.30

The following intermediates in Table 36 were synthesized according to the above procedure:

TABLE 36

| Inter- | | | | |
|---|---|---|---|---|
| mediate | Structure | Name | [(M + 1)]⁺ | ¹H NMR |
| 140-1 | 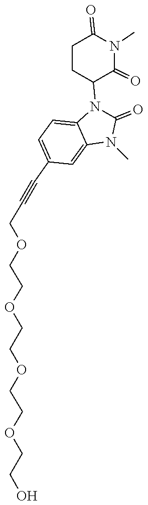 [SMILES: CN1C(=O)CCC(n2c(=O)n(C)c3cc(C#CCOCCOCCOCCOCCO)ccc32)C1=O] | 3-[5-(1-hydroxy- 3,6,9,12- tetraoxapentadec- 14-yn-15-yl)-3- methyl-2-oxo-2,3- dihydro-1H-1,3- benzodiazol-1-yl]- 1- methylpiperidine- 2,6-dione | 504.20 | (400 MHz, CD₃OD) δ 7.32-7.26 (m, 1H), 7.22 (t, J = 8.1 Hz, 1H), 7.08 (t, J = 7.5 Hz, 1H), 5.46-5.35 (m, 1H), 4.87 (s, 1H), 4.44 (t, J = 3.6 Hz, 2H), 3.81-3.74 (m, 2H), 3.67 (d, J = 6.1 Hz, 11H), 3.56 (q, J = 5.0 Hz, 2H), 3.43 (d, J = 7.2 Hz, 3H), 3.18 (d, J = 6.5 Hz, 3H), 3.00- 2.90 (m, 2H), 2.85-2.70 (m, 1H), 2.20-2.08 (m, 1H) |
| 140-2 | 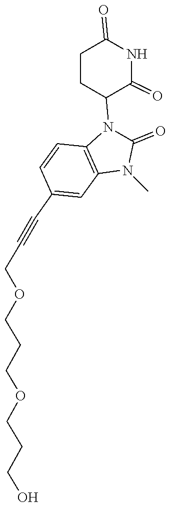 [SMILES: Cn1c(=O)n(C2CCC(=O)NC2=O)c2ccc(C#CCOCCCOCCCO)cc21] | 3-[5-[3-(3- hydroxypropoxy)prop- 1-yn-1-yl]-3- methyl-2-oxo-2,3- dihydro-1H-1,3- benzodiazol-1- yl]piperidine-2,6- dione | 430.15 | (400 MHz, DMSO-d₆) δ 11.14 (s, 1H), 7.34 (d, J = 1.4 Hz, 1H), 7.24- 7.10 (m, 2H), 5.50-5.30 (m, 1H), 4.41-4.34 (m, 3H), 3.57 (t, J = 6.4 Hz, 2H), 3.45-3.40 (m, 6H), 3.00- 2.85 (m, 1H), 2.78-2.59 (m, 2H), 2.10-1.97 (m, 1H), 1.85-1.70 (m, 2H), 1.68-1.50 (m, 2H) |
| 140-3 | 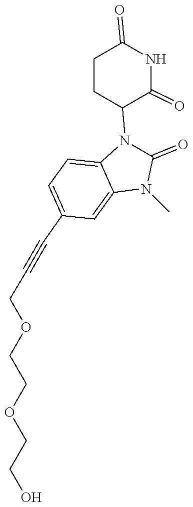 [SMILES: Cn1c(=O)n(C2CCC(=O)NC2=O)c2ccc(C#CCOCCOCCO)cc21] | 3-(5-[3-[2-(2- hydroxyethoxy)eth- oxy]prop-1-yn-1- yl]-3-methyl-2- oxo-2,3-dihydro- 1H-1,3- benzodiazol-1- yl)piperidine-2,6- dione | 402.20 | (400 MHz, DMSO-d₆) δ 11.13 (s, 1H), 7.35 (d, J = 1.4 Hz, 1H), 7.23- 7.11 (m, 2H), 5.45-5.35 (m, 1H), 4.61 (t, J = 5.5 Hz, 1H), 4.40 (s, 2H), 3.68-3.62 (m, 2H), 3.65-3.48 (m, 2H), 3.55-3.48 (m, 2H), 3.46- 3.38 (m, 2H), 3.35 (s, 3H), 2.96- 2.83 (m, 1H), 2.77-2.59 (m, 2H), 2.10-2.00 (m, 1H) |
| 140-4 | 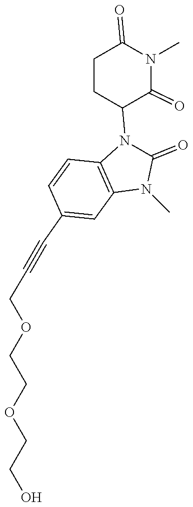 [SMILES: CN1C(=O)CCC(n2c(=O)n(C)c3cc(C#CCOCCOCCO)ccc32)C1=O] | 3-(5-[3-[2-(2- Hydroxyethoxy)eth- oxy]prop-1-yn-1- yl]-3-methyl-2- oxo-2,3-dihydro- 1H-1,3- benzodiazol-1-yl)- 1- methylpiperidine- 2,6-dione | 416.35 | (400 MHz, DMSO-d₆) δ 7.35 (s, 1H), 7.16 (s, 2H), 5.50-5.40 (m, 1H), 4.61 (t, J = 5.4 Hz, 1H), 4.40 (s, 2H), 3.69-3.61 (m, 2H), 3.60- 3.50 (m, 2H), 3.54-3.47 (m, 2H), 3.45 (d, J = 5.0 Hz, 2H), 3.34 (s, 2H), 3.04 (s, 3H), 3.00-2.91 (m, 1H), 2.85-2.64 (m, 2H), 2.07-1.98 (m, 1H), 1.24 (s, 1H) |
| 140-5 | 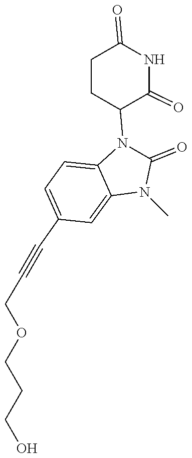 [SMILES: Cn1c(=O)n(C2CCC(=O)NC2=O)c2ccc(C#CCOCCCO)cc21] | 3-[5-[3-(3- hydroxypropoxy)prop- 1-yn-1-yl]-3- methyl-2-oxo-2,3- dihydro-1H-1,3- benzodiazol-1- yl]piperidine-2,6- dione | 372.25 | (400 MHz, DMSO-d₆) δ 1.14 (s, 1H), 7.34 (d, J = 1.3 Hz, 1H), 7.23- 7.16 (m, 1H), 7.14 (d, J = 8.2 Hz, 1H), 5.45-5.35 (m, 1H), 4.46 (t, J = 5.1 Hz, 1H), 4.36 (s, 2H), 3.58 (t, J = 6.5 Hz, 2H), 3.50-3.40 (m, 3H), 2.96-2.83 (m, 1H), 2.79-2.58 (m, 2H), 2.08-1.98 (m, 1H), 1.80-1.60 (m, 2H) |
| 140-6 | 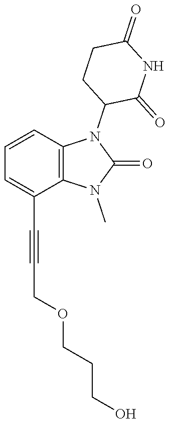 [SMILES: Cn1c(=O)n(C2CCC(=O)NC2=O)c2cccc(C#CCOCCCO)c21] | 3-[4-[3-(3- hydroxypropoxy)prop- 1-yn-1-yl]-3- methyl-2-oxo-1,3- benzodiazol-1- yl]piperidine-2,6- dione | 372.30 | (400 MHz, DMSO-d₆) δ 11.13 (s, 1H), 7.18 (d, J = 7.8 Hz, 1H), 7.13 (d, J = 7.7 Hz, 1H), 7.04 (t, J = 7.9 Hz, 1H), 5.41 (d, J = 5.3 Hz, 1H), 4.44 (d, J = 5.1 Hz, 1H), 4.42 (s, 2H), 3.60 (t, J = 6.5 Hz, 3H), 3.48 (q, J = 6.0 Hz, 3H), 2.96-2.83 (m, 1H), 2.78-2.59 (m, 2H), 2.15-1.94 (m, 2H), 1.80-1.65 (m, 2H) |
| 140-7 | 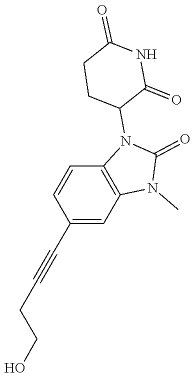 [SMILES: Cn1c(=O)n(C2CCC(=O)NC2=O)c2ccc(C#CCCO)cc21] | 3-[5-(4- hydroxybut-1-yn- 1-yl)-3-methyl-2- oxo-1,3- benzodiazol-1- yl]piperidine-2,6- dione | 328.15 | (400 MHz, DMSO-d₆) δ 11.11 (s, 1H), 7.25 (d, J = 1.3 Hz, 1H), 7.10 (d, J = 1.1 Hz, 2H), 5.45-5.30 (m, 1H), 4.88 (s, 1H), 3.60 (d, J = 7.7 Hz, 2H), 3.34 (s, 3H), 2.95-2.83 (m, 1H), 2.75-2.59 (m, 2H), 2.55 (t, J = 3.5 Hz, 2H), 2.10-1.95 (m, 1H) |
| 140-8 | 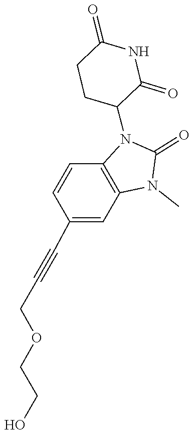 [SMILES: Cn1c(=O)n(C2CCC(=O)NC2=O)c2ccc(C#CCOCCO)cc21] | 3-[5-[3-(2- hydroxyethoxy)prop- 1-yl-1-yl]-3- methyl-2-oxo-2,3- dihydro-1H-1,3- benzodiazol-1- yl]piperidine-2,6- dione | 358.30 | (400 MHz, DMSO-d₆) δ 11.13 (s, 1H), 7.34 (d, J = 1.4 Hz, 1H), 7.23- 7.08 (m, 2H), 5.45-5.30 (m, 1H), 4.68 (d, J = 5.6 Hz, 1H), 4.40 (s, 2H), 3.55 (d, J = 2.2 Hz, 4H), 3.35 (s, 3H), 2.99-2.82 (m, 1H), 2.78- 2.58 (m, 2H), 2.07-1.95 (m, 1H) |
| 140-9 | 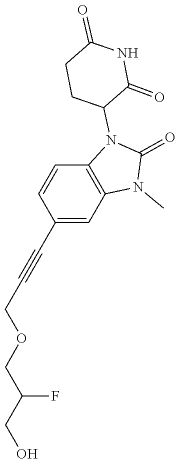 [SMILES: Cn1c(=O)n(C2CCC(=O)NC2=O)c2ccc(C#CCOCC(F)CO)cc21] | 3-[5-[3-(2-fluoro- 3- hydroxypropoxy)prop- 1-yn-1-yl]-3- methyl-2-oxo-1,3- benzodiazol-1- yl]piperidine-2,6- dione | 390.30 | (400 MHz, DMSO-d₆) δ 11.12 (s, 1H), 7.35 (d, J = 1.5 Hz, 1H), 7.26- 7.12 (m, 2H), 5.45-5.30 (m, 1H), 4.98 (t, J = 5.7 Hz, 1H), 4.75-4.56 (m, 1H), 4.44 (s, 2H), 3.86-3.50 (m, 4H), 3.36 (s, 3H), 2.95-2.80 (m, 1H), 2.79-2.58 (m, 2H), 2.10-1.95 (m, 1H) |
| 140- 10 | 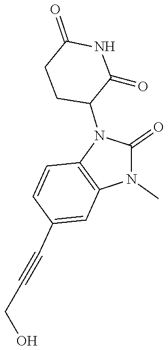 [SMILES: Cn1c(=O)n(C2CCC(=O)NC2=O)c2ccc(C#CCO)cc21] | 3-[5-(3- hydroxyprop-1-yn- 1-yl)-3-methyl-2- oxo-2,3-dihydro- 1H-1,3- benzodiazol-1- yl]piperidine-2,6- dione | 314.20 | (400 MHz, DMSO-d₆) δ 11.13 (s, 1H), 7.30 (s, 1H), 7.14 (s, 2H), 5.45-5.35 (m, 1H), 5.31 (t, J = 6.0 Hz, 1H), 4.31 (d, J = 5.7 Hz, 2H), 3.35 (s, 3H), 2.98-2.82 (m, 1H), 2.79-2.57 (m, 2H), 2.03 (d, J = 10.8 Hz, 1H) |
| 140- 11 | 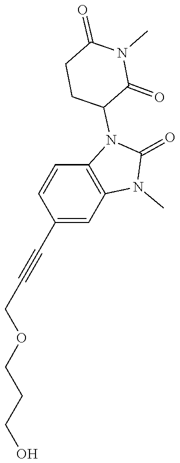 [SMILES: CN1C(=O)CCC(n2c(=O)n(C)c3cc(C#CCOCCCO)ccc32)C1=O] | 3-[5-[3-(3- hydroxypropoxy)prop- 1-yn-1-yl]-3- methyl-2-oxo-1,3- benzodiazol-1-yl]- 1- methylpiperidine- 2,6-dione | 386.10 | (400 MHz, DMSO-d₆) δ 7.34 (s, 1H), 7.15 (d, J = 1.1 Hz, 2H), 5.50- 5.38 (m, 1H), 4.43 (t, J = 5.1 Hz, 1H), 4.35 (s, 2H), 3.58 (t, J = 6.5 Hz, 2H), 3.48 (td, J = 6.3, 5.1 Hz, 2H), 3.36 (s, 3H), 3.04 (s, 3H), 3.01-2.91 (m, 1H), 2.83-2.64 (m, 2H), 2.08-2.00 (m, 1H), 1.75-1.65 (m, 2H) |
| 140- 12 | 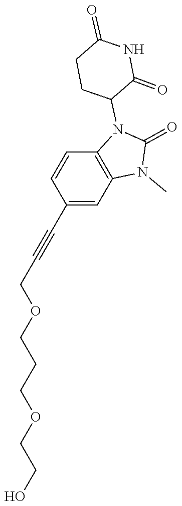 [SMILES: Cn1c(=O)n(C2CCC(=O)NC2=O)c2ccc(C#CCOCCCOCCO)cc21] | 3-(5-[3-[3-(2- hydroxyethoxy)pro- poxy]prop-1-yn-1- yl]-3-methyl-2- oxo-2,3-dihydro- 1H-1,3- benzodiazol-1- yl)piperidine-2,6- dione | 416.30 | (400 MHz, DMSO-d₆) δ 11.14 (s, 1H), 7.34 (d, J = 1.4 Hz, 1H), 7.23- 7.11 (m, 2H), 5.45-5.35 (m, 1H), 4.58 (t, J = 5.5 Hz, 1H), 4.36 (s, 2H), 3.58 (t, J = 6.4 Hz, 2H), 3.52- 3.44 (m, 4H), 3.40 (d, J = 5.2 Hz, 2H), 3.35 (s, 3H), 2.99-2.83 (m, 1H), 2.78-2.60 (m, 2H), 2.10-1.98 (m, 1H), 1.85-1.65 (m, 2H) |

### Step 3: 3-[5-[3-(4-hydroxybutoxy)propyl]-3-methyl-2-oxo-2,3-dihydro-1H-1,3-benzodiazol-1-yl]piperidine-2,6-dione

To a stirred solution of 3-[5-[3-(4-hydroxybutoxy)prop-1-yn-1-yl]-3-methyl-2-oxo-2,3-dihydro-1H-1,3-benzodiazol-1-yl]piperidine-2,6-dione (1.00 g, 2.60 mmol) in THF (30.0 mL) was added 10% palladium on activated carbon (100 mg) at room temperature. The resulting mixture was stirred for 16 h at room temperature under hydrogen atmosphere. Upon completion, the resulting mixture was filtered. The filter cake was washed with EtOAc (3×5.00 mL). The combined filtrates was concentrated under reduced pressure to afford 3-[5-[3-(4-hydroxybutoxy)propyl]-3-methyl-2-oxo-2,3-dihydro-1H-1,3-benzodiazol-1-yl]piperidine-2,6-dione (1.00 g, 99%) as a white solid. ¹H NMR (400 MHz, DMSO-d₆) δ 11.08 (s, 1H), 7.09-6.98 (m, 2H), 6.90-6.84 (m, 1H), 5.40-5.30 (m, 1H), 4.37 (t, J=5.2 Hz, 1H), 3.45-3.34 (m, 6H), 3.33 (s, 3H), 2.97-2.84 (m, 1H), 2.78-2.59 (m, 4H), 2.10-1.96 (m, 1H), 1.86-1.77 (m, 2H), 1.53-1.42 (m, 4H). LC/MS (ESI, m/z): [(M+1)]⁺=390.30.

The following intermediates in Table 37 were synthesized according to the above procedure:

TABLE 37

| Inter- | | | | |
|---|---|---|---|---|
| medi- | | | MS: | |
| ate | Structure | Name | [(M + 1)]⁺ | ¹H NMR |
| T17 | 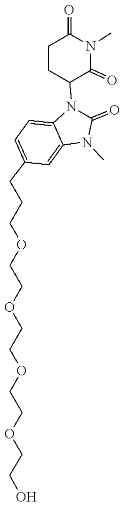 [SMILES: CN1C(=O)CCC(n2c(=O)n(C)c3cc(CCCOCCOCCOCCOCCO)ccc32)C1=O] | 3-[5-(1-hydroxy- 3,6,9,12- tetraoxapentadecan- 15-yl)-3-methyl-2- oxo-2,3-dihydro- 1H-1,3- benzodiazol-1-yl]- 1-methylpiperidine- 2,6-dione | 508.40 | (400 MHz, CDCl₃) δ 6.86-6.80 (m, 2H), 6.61 (d, J = 8.3 Hz, 1H), 5.15- 5.05 (m, 1H), 3.75-3.70 (m, 2H), 3.68-3.55 (m, 9H), 3.54-3.45 (m, 4H), 3.40 (t, J = 6.4 Hz, 2H), 3.36 (s, 3H), 3.17 (s, 3H), 3.01-2.90 (m, 1H), 2.84-2.57 (m, 4H), 2.20 (s, 1H), 2.16-2.00 (m, 1H), 1.91-.78 (m, 2H), 1.36 (s, 1H) |
| T18 | 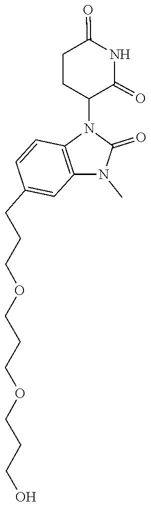 [SMILES: Cn1c(=O)n(C2CCC(=O)NC2=O)c2ccc(CCCOCCCOCCCO)cc21] | 3-(5-[3-[3-(3- hydroxypropoxy)pro- poxy]propyl]-3- methyl-2-oxo-2,3- dihydro-1H-1,3- benzodiazol-1- yl)piperidine-2,6- dione | 434.15 | (400 MHz, DMSO-d₆) δ 11.09 (s, 1H), 7.06-6.97 (m, 2H), 6.86 (d, J = 7.9 Hz, 1H), 5.40-5.30 (m, 1H), 4.45-4.35 (m, 1H), 3.48-3.35 (m, 10H), 2.95-2.83 (m, 1H), 2.70-2.55 (m, 4H), 2.00 (d, J = 9.9 Hz, 1H), 1.81 (t, J = 7.3 Hz, 2H), 1.80-1.68 (m, 2H), 1.65-1.59 (m, 2H) |
| T19 | 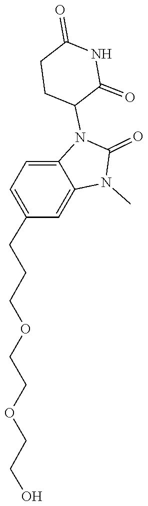 [SMILES: Cn1c(=O)n(C2CCC(=O)NC2=O)c2ccc(CCCOCCOCCO)cc21] | 3-(5-[3-[2-(2- hydroxyethoxy)eth- oxy]propyl]-3- methyl-2-oxo-2,3- dihydro-1H-1,3- benzodiazol-1- yl)piperidine-2,6- dione | 406.35 | (400 MHz, DMSO-d₆) δ 11.09 (s, 1H), 7.05 (d, J = 1.5 Hz, 1H), 7.01 (d, J = 8.0 Hz, 1H), 6.95-6.80 (m, 1H), 5.40-5.30 (m, 1H), 4.59 (t, J = 5.4 Hz, 1H), 3.56-3.52 (m, 2H), 3.52-3.46 (m, 4H), 3.46-3.37 (m, 4H), 3.33 (s, 3H), 3.00-2.85 (m, 1H), 2.78-2.57 (m, 4H), 2.05-1.90 (m, 1H), 1.88-1.76 (m, 2H) |
| T20 | 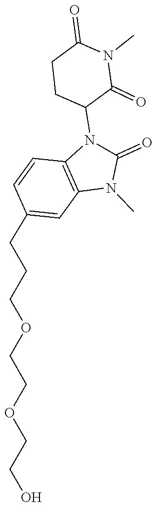 [SMILES: CN1C(=O)CCC(n2c(=O)n(C)c3cc(CCCOCCOCCO)ccc32)C1=O] | 3-(5-[3-[2-(2- Hydroxyethoxy)eth- oxy]propyl]-3- methyl-2-oxo-2,3- dihydro-1H-1,3- benzodiazol-1-yl)- 1-methylpiperidine- 2,6-dione | 420.20 | (400 MHz, DMSO-d₆) δ 7.10-6.97 (m, 2H), 6.90-6.80 (m, 1H), 5.45- 5.34 (m, 1H), 4.59 (s, 1H), 3.58- 3.47 (m, 6H), 3.46-3.37 (m, 4H), 3.04 (s, 3H), 3.01-2.91 (m, 1H), 2.87-2.56 (m, 4H), 2.00 (s, 3H), 1.90-1.75 (m, 2H), 1.24 (d, J = 3.9 Hz, 1H) |
| T21 | 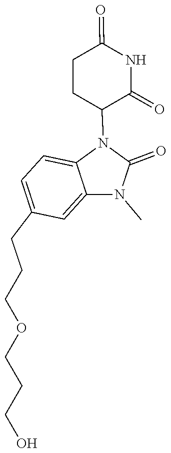 [SMILES: Cn1c(=O)n(C2CCC(=O)NC2=O)c2ccc(CCCOCCCO)cc21] | 3-[5-[3-(3- hydroxypropoxy)pro- pyl]-3-methyl-2- oxo-2,3-dihydro- 1H-1,3- benzodiazol-1- yl]piperidine-2,6- dione | 376.30 | (400 MHz, DMSO-d₆) δ 11.10 (s, 1H), 7.11-6.98 (m, 2H), 6.95-6.70 (m, 1H), 5.40-5.30 (m, 1H), 4.40 (t, J = 5.1 Hz, 1H), 3.51-3.46 (m, 2H), 3.42 (t, J = 6.4 Hz, 2H), 3.37 (d, J = 6.4 Hz, 2H), 3.33 (s, 3H), 2.9- 2.84 (m, 1H), 2.75-2.58 (m, 4H), 2.00 (m, 1H), 1.85-1.70 (m, 2H), 1.68-1.60 (m, 2H) |
| T22 | 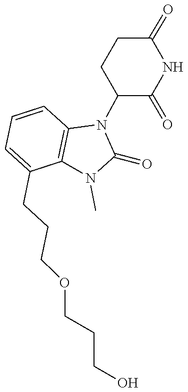 [SMILES: Cn1c(=O)n(C2CCC(=O)NC2=O)c2cccc(CCCOCCCO)c21] | 3-[4-[3-(3- hydroxypropoxy)pro- pyl]-3-methyl-2- oxo-1,3- benzodiazol-1- yl]piperidine-2,6- dione | 376.40 | (400 MHz, DMSO-d₆) δ 11.09 (s, 1H), 6.97 (d, J = 4.8 Hz, 2H), 6.88 (q, J = 4.4 Hz, 1H), 5.45-5.34 (m, 1H), 4.39 (t, J = 5.1 Hz, 1H), 3.57 (s, 3H), 3.03-2.94 (m, 1H), 2.94- 2.79 (m, 2H), 2.78-2.56 (m, 2H), 2.51 (t, J = 2.9 Hz, 3H), 2.12-1.89 (m, 1H), 1.86-1.80 (m, 2H), 1.14- 1.60 (m, 2H) |
| T23 | 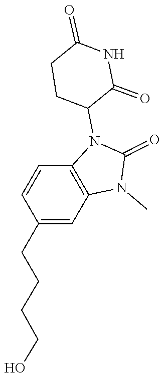 [SMILES: Cn1c(=O)n(C2CCC(=O)NC2=O)c2ccc(CCCCO)cc21] | 3-[5-(4- hydroxybutyl)-3- methyl-2-oxo-1,3- benzodiazol-1- yl]piperidine-2,6- dione | 332.20 | (400 MHz, DMSO-d₆) δ 11.09 (s, 1H), 7.05-6.97 (m, 2H), 6.90-6.80 (m, 1H), 5.40-5.30 (m, 1H), 4.37 (t, J = 5.2 Hz, 1H), 3.46-3.30 (m, 5H), 2.97-2.84 (m, 1H), 2.78-2.69 (m, 1H), 2.66-2.50 (m, 3H), 2.05-1.92 (m, 1H), 1.70-1.50 (m, 2H), 1.45- 1.35 (m, 2H) |
| T24 | 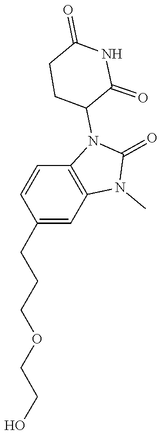 [SMILES: Cn1c(=O)n(C2CCC(=O)NC2=O)c2ccc(CCCOCCO)cc21] | 3-[5-[3-(2- hydroxyethoxy)pro- pyl]-3-methyl-2- oxo-2,3-dihydro- 1H-1,3- benzodiazol-1- yl]piperidine-2,6- dione | 362.30 | (400 MHz, DMSO-d₆) δ 11.10 (s, 1H), 7.11-6.98 (m, 2H), 6.90-6.80 (m, 1H), 5.40-5.30 (m, 1H), 4.60 (t, J = 5.5 Hz, 1H), 3.61-3.31 (m, 9H), 2.95-2.83 (m, 1H), 2.78-2.57 (m, 4H), 2.06-1.95 (m, 1H), 1.88-1.76 (m, 2H). |
| T25 | 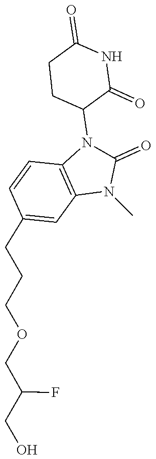 [SMILES: Cn1c(=O)n(C2CCC(=O)NC2=O)c2ccc(CCCOCC(F)CO)cc21] | 3-[5-[3-(2-fluoro-3- hydroxypropoxy)pro- pyl]-3-methyl-2- oxo-1,3- benzodiazol-1- yl]piperidine-2,6- dione | 394.30 | (400 MHz, CDCl₃) δ 8.17 (s, 1H), 6.95-6.85 (m, 2H), 6.74 (d, J = 8.0 Hz, 1H), 5.30-5.15 (m, 1H), 4.85- 4.60 (m, 1H), 3.96-3.79 (m, 2H), 3.79-3.61 (m, 2H), 3.52 (t, J = 6.2 Hz, 2H), 3.45 (s, 3H), 3.02-2.92 (m, 1H), 2.91-2.61 (m, 4H), 2.26 (m, 1H), 2.00-1.88 (m, 2H) |
| T26 | 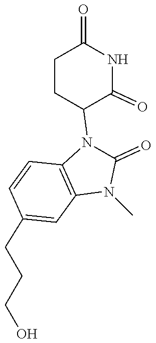 [SMILES: Cn1c(=O)n(C2CCC(=O)NC2=O)c2ccc(CCCO)cc21] | 3-[5-(3- hydroxypropyl)-3- methyl-2-oxo-2,3- dihydro-1H-1,3- benzodiazol-1- yl]piperidine-2,6- dione | 318.10 | (400 MHz, DMSO-d₆) δ 11.12 (d, J = 12.6 Hz, 1H), 7.14-6.96 (m, 2H), 6.89 (t, J = 9.9 Hz, 1H), 5.45-5.34 (m, 1H), 4.58-4.38 (m, 1H), 3.70- 3.60 (m, 2H), 3.50-3.40 (m, 2H), 3.34 (s, 3H), 3.00-2.85 (m, 1H), 2.60-2.45 (m, 2H), 2.17-1.94 (m, 1H), 1.80-1.70 (m, 2H) |
| T27 | 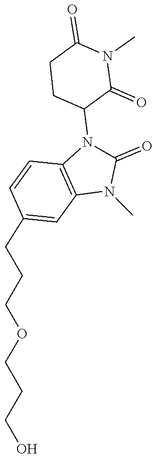 [SMILES: CN1C(=O)CCC(n2c(=O)n(C)c3cc(CCCOCCCO)ccc32)C1=O] | 3-[5-[3-(3- hydroxypropoxy)pro- pyl]-3-methyl-2- oxo-1,3- benzodiazol-1-yl]- 1-methylpiperidine- 2,6-dione | 390.25 | (400 MHz, DMSO-d₆) δ 7.07-6.98 (m, 2H), 6.90-6.80 (m, 1H), 5.46- 5.35 (m, 1H), 4.39 (t, J = 5.1 Hz, 1H), 3.52-3.40 (m, 4H), 3.39-3.33 (m, 5H), 3.04 (s, 3H), 3.02-2.92 (m, 1H), 2.84-2.75 (m, 1H), 2.74-2.61 (m, 3H), 2.07-1.98 (m, 1H), 1.86- 1.76 (m, 2H), 1.70-1.60 (m, 2H) |
| T28 | 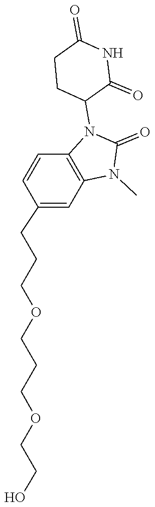 [SMILES: Cn1c(=O)n(C2CCC(=O)NC2=O)c2ccc(CCCOCCCOCCO)cc21] | 3-(5-[3-[3-(2- hydroxyethoxy)pro- poxy]propyl]-3- methyl-2-oxo-2,3- dihydro-1H-1,3- benzodiazol-1- yl)piperidine-2,6- dione | 420.35 | (400 MHz, DMSO-d₆) δ 11.10 (s, 1H), 7.10-6.96 (m, 2H), 6.90-6.80 (m, 1H), 5.40-5.30 (m, 1H), 4.58 (t, J = 5.5 Hz, 1H), 3.53-3.35 (m, 10H), 3.33 (s, 3H), 2.95-2.84 (m, 1H), 2.76-2.61 (m, 4H), 2.06-1.95 (m, 1H), 1.85-1.71 (m, 4H) |

### Step 4: 4-[3-[1-(2,6-dioxopiperidin-3-yl)-3-methyl-2-oxo-2,3-dihydro-1H-1,3-benzodiazol-5-yl]propoxy]butanal

To a stirred solution of oxalyl chloride (1.30 g, 10.3 mmol) in DCM (60.0 mL) was added DMSO (1.00 g, 12.8 mmol) dropwise at −78° C. under nitrogen atmosphere. The resulting solution was stirred for 30 min at −78° C. Then a solution of 3-[5-[3-(4-hydroxybutoxy)propyl]-3-methyl-2-oxo-2,3-dihydro-1H-1,3-benzodiazol-1-yl]piperidine-2,6-dione (1.00 g, 2.57 mmol) in DCM (5.00 mL) was added dropwise at −78° C. The resulting solution was stirred for another 30 min at the same temperature. Then TEA (2.60 g, 25.7 mmol) was added at −78° C. The reaction temperature was slowly increased to room temperature in 30 min. The resulting mixture was diluted with water (30.0 mL) and extracted with DCM (3×30.0 mL). The combined organic layers were dried over anhydrous Na₂SO₄. After filtration, the filtrate was concentrated under reduced pressure. The residue was purified by reversed phase flash chromatography with the following conditions: Column: WelFlash™ C18-I, 20-40 um, 330 g; Eluent A: water (plus 10 mmol/L FA); Eluent B: ACN; Gradient: 15%-35% B in 25 min; Flow rate: 80 mL/min; Detector: UV 220/254 nm; desired fractions were collected at 27% B and concentrated under reduced pressure to afford 4-[3-[1-(2,6-dioxopiperidin-3-yl)-3-methyl-2-oxo-2,3-dihydro-1H-1,3-benzodiazol-5-yl]propoxy]butanal (670 mg, 67%) as a white solid. ¹H NMR (400 MHz, DMSO-d₆) δ 11.10 (s, 1H), 9.68 (t, J=1.7 Hz, 1H), 7.06-6.98 (m, 2H), 6.90-6.80 (m, 1H), 5.40-5.30 (m, 1H), 3.36 (d, J=5.8 Hz, 4H), 3.33 (s, 3H), 2.97-2.84 (m, 1H), 2.78-2.58 (m, 4H), 2.50-2.40 (m, 2H), 2.10-1.95 (m, 1H), 1.86-1.73 (m, 4H). LC/MS (ESI, m/z): [(M+1)]⁺=388.30

The following intermediates in Table 38 were synthesized according to the above procedure:

TABLE 38

| Inter- | | | | |
|---|---|---|---|---|
| medi- | | | MS: | |
| ate | Structure | Name | [(M + 1)]⁺ | ¹H NMR |
| S3 | 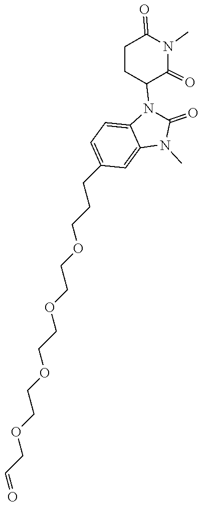 [SMILES: CN1C(=O)CCC(n2c(=O)n(C)c3cc(CCCOCCOCCOCCOCC=O)ccc32)C1=O] | 15-[3-methyl-1-(1- methyl-2,6- dioxopiperidin-3- yl)-2-oxo-2,3- dihydro-1H-1,3- benzodiazol-5-yl]- 3,6,9,12- tetraoxapentadecan- al | 506.30 | (400 MHz, CDCl₃) δ 6.82 (m, J = 1.6 Hz, 2H), 6.61 (d, J = 8.3 Hz, 1H), 5.20- 5.05 (m, 1H), 3.75-3.65 (m, 2H), 3.60 (d, J = 3.1 Hz, 9H), 3.58-3.25 (m, 4H), 3.40 (t, J = 6.4 Hz, 2H), 3.36 (s, 3H), 3.17 (s, 3H), 3.01-2.90 (m, 1H), 2.84- 2.57 (m, 4H), 2.20 (s, 1H), 2.16-2.05 (m, 1H), 1.91-1.78 (m, 2H), 1.36 (s, 1H) |
| S4 | 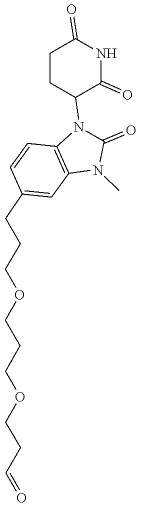 [SMILES: Cn1c(=O)n(C2CCC(=O)NC2=O)c2ccc(CCCOCCCOCCC=O)cc21] | 3-(3-[3-[1-(2,6- dioxopiperidin-3- yl)-3-methyl-2- oxo-2,3-dihydro- 1H-1,3- benzodiazol-5- yl]propoxy]propoxy) propanal | 432.35 | (400 MHz, DMSO-d₆) δ 11.10 (s, 1H), 9.65 (t, J = 2.1 Hz, 1H), 7.11-6.98 (m, 2H), 6.90-6.80 (m, 1H), 5.40-5.30 (m, 1H), 3.69 (t, J = 6.0 Hz, 2H), 3.49-3.33 (m, 9H), 2.95-2.80 (m, 1H), 2.77-2.57 (m, 6H), 2.05-1.90 (m, 1H), 1.87-1.77 (m, 2H), 1.75-1.60 (m, 2H) |
| S5 | 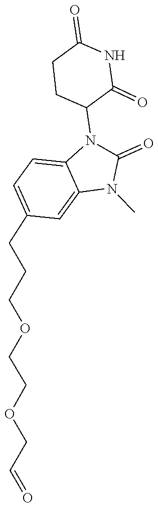 [SMILES: Cn1c(=O)n(C2CCC(=O)NC2=O)c2ccc(CCCOCCOCC=O)cc21] | 2-(2-[3-[1-(2,6- dioxopiperidin-3- yl)-3-methyl-2- oxo-2,3-dihydro- 1H-1,3- benzodiazol-5- yl]propoxy]ethoxy) acetaldehyde | 404.25 | (400 MHz, DMSO-d₆) δ 11.10 (s, 1H), 9.59 (s, 1H), 7.10-6.96 (m, 2H), 6.90- 6.80 (m, 1H), 5.40-5.30 (m, 1H), 4.21 (s, 1H), 3.66-3.61 (m, 1H), 3.59-3.45 (m, 3H), 3.41 (t, J = 6.4 Hz, 2H), 3.33 (s, 3H), 2.97-2.84 (m, 1H), 2.78-2.58 (m, 4H), 2.09-1.93 (m, 1H), 1.85-1.70 (m, 2H) |
| S6 | 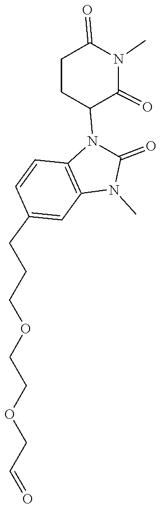 [SMILES: CN1C(=O)CCC(n2c(=O)n(C)c3cc(CCCOCCOCC=O)ccc32)C1=O] | 2-(2-[3-[3-Methyl- 1-(1-methyl-2,6- dioxopiperidin-3- yl)-2-oxo-2,3- dihydro-1H-1,3- benzodiazol-5- yl]propoxy]ethoxy) acetaldehyde | 418.30 | (400 MHz, DMSO-d₆) δ 9.59 (s, 1H), 7.09-6.96 (m, 2H), 6.90-6.80 (m, 1H), 5.45-5.35 (m, 1H), 4.21 (s, 1H), 3.70- 3.60 (m, 2H), 3.59-3.45 (m, 3H), 3.41 (t, J = 6.4 Hz, 2H), 3.33 (s, 3H), 3.03 (d, J = 2.8 Hz, 3H), 3.00-2.91 (m, 1H), 2.84- 2.55 (m, 4H), 2.10-1.95 (m, 1H), 1.89- 1.70 (m, 2H) |
| S7 | 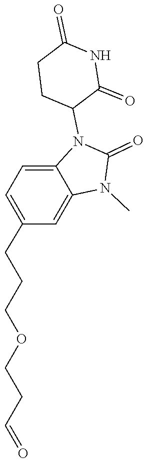 [SMILES: Cn1c(=O)n(C2CCC(=O)NC2=O)c2ccc(CCCOCCC=O)cc21] | 3-[3-[1-(2,6- dioxopiperidin-3- yl)-3-methyl-2- oxo-2,3-dihydro- 1H-1,3- benzodiazol-5- yl]propoxy]propan- al | 374.15 | (400 MHz, DMSO-d₆) δ 11.09 (s, 1H), 9.68 (t, J = 2.0 Hz, 1H), 7.06-6.98 (m, 2H), 6.87 (d, J = 7.9 Hz, 1H), 5.40-5.30 (m, 1H), 3.71 (t, J = 6.0 Hz, 2H), 3.40 (d, J = 6.4 Hz, 4H), 2.93-2.84 (m, 1H), 2.63 (td, J = 6.2, 2.3 Hz, 4H), 2.04-1.97 (m, 1H), 1.85-1.70 (m, 2H) |
| S8 | 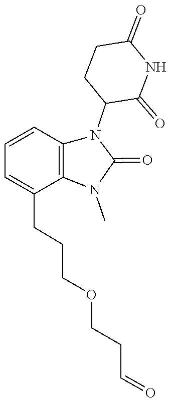 | 3-[3-[1-(2,6- dioxopiperidin-3- yl)-3-methyl-2- oxo-1,3- benzodiazol-4- yl]propoxy]propan- al | 374.40 | (400 MHz, DMSO-d₆) δ 11.09 (s, 1H), 9.69 (t, J = 2.0 Hz, 1H), 6.97 (d, J = 4.8 Hz, 2H), 6.90-6.80 (m, 1H), 5.40-5.30 (m, 1H), 3.73 (t, J = 6.0 Hz, 2H), 3.50- 3.40 (m, 3H), 3.32 (s, 1H), 2.91-2.80 (m, 1H), 2.78-2.57 (m, 4H), 2.48 (s, 1H), 2.05-1.90 (m, 1H), 1.85-1.74 (m, 2H) |
| S9 | 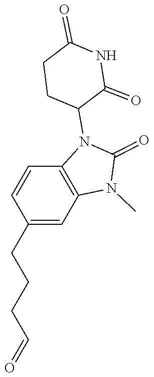 [SMILES: Cn1c(=O)n(C2CCC(=O)NC2=O)c2ccc(CCCC=O)cc21] | 4-[1-(2,6- dioxopiperidin-3- yl)-3-methyl-2- oxo-2,3-dihydro- 1H-1,3- benzodiazol-5- yl]butanal | 330.30 | (400 MHz, DMSO-d₆) δ 11.09 (s, 1H), 9.68 (d, J = 1.7 Hz, 1H), 7.07-6.97 (m, 2H), 6.90-6.80 (m, 1H), 5.40-5.30 (m, 1H), 3.34 (s, 3H), 2.97-2.84 (m, 1H), 2.66-2.58 (m, 2H), 2.50-2.45 (m, 2H), 2.43-2.33 (m, 2H), 1.99 (s, 1H), 1.90- 1.70 (m, 2H) |
| S10 | 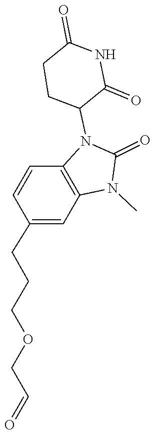 [SMILES: Cn1c(=O)n(C2CCC(=O)NC2=O)c2ccc(CCCOCC=O)cc21] | 2-[3-[1-(2,6- dioxopiperidin-3- yl)-3-methyl-2- oxo-2,3-dihydro- 1H-1,3- benzodiazol-5- yl]propoxy]acetalde- hyde | 360.15 | (400 MHz, DMSO-d₆) δ 11.10 (s, 1H), 9.60 (s, 1H), 7.15-6.96 (m, 2H), 6.94- 6.81 (m, 1H), 5.40-5.30 (m, 1H), 4.18 (s, 1H), 3.49-3.35 (m, 7H), 2.90 (m, 1H), 2.78-2.57 (m, 4H),, 2.06-1.95 (m, 1H), 1.90-1.75 (m, 2H) |
| S11 | 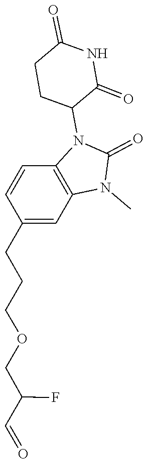 [SMILES: Cn1c(=O)n(C2CCC(=O)NC2=O)c2ccc(CCCOCC(F)C=O)cc21] | 3-[3-[1-(2,6- dioxopiperidin-3- yl)-3-methyl-2- oxo-1,3- benzodiazol-5- yl]propoxy]-2- fluoropropanal | 392.30 | (400 MHz, CDCl3) δ 9.84 (d, J = 5.8 Hz, 1H), 8.44 (s, 1H), 6.93-6.85 (m, 2H), 6.74 (d, J = 7.9 Hz, 1H), 5.40-5.30 (m, 1H), 5.00-4.90 (m, 1H), 3.98-3.82 (m, 2H), 3.60-3.48 (m, 2H), 3.45 (s, 3H), 3.00-2.90 (m, 1H), 2.86 (d, J = 5.0 Hz, 1H), 2.76-2.69 (m, 3H), 2.03 (s, 1H), 1.95-1.80 (m, 2H) |
| S12 | 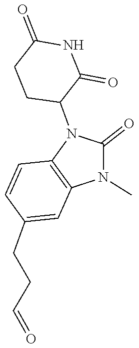 [SMILES: Cn1c(=O)n(C2CCC(=O)NC2=O)c2ccc(CCC=O)cc21] | 3-[1-(2,6- dioxopiperidin-3- yl)-3-methyl-2- oxo-2,3-dihydro- 1H-1,3- benzodiazol-5- yl]propanal | 316.30 | (400 MHz, DMSO-d₆) δ 11.09 (s, 1H), 9.73 (d, J = 1.6 Hz, 1H), 7.08 (d, J = 1.5 Hz, 1H), 7.02 (d, J = 8.0 Hz, 1H), 6.90 (m, 1H), 5.40-5.30 (m, 1H), 3.34 (s, 3H), 2.95-2.80 (m, 3H), 2.84-2.76 (m, 2H), 2.77-2.56 (m, 2H), 2.05-1.95 (m, 1H) |
| S13 | 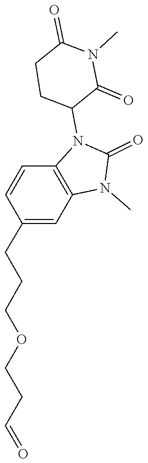 [SMILES: CN1C(=O)CCC(n2c(=O)n(C)c3cc(CCCOCCC=O)ccc32)C1=O] | 3-[3-[3-methyl-1- (1-methyl-2,6- dioxopiperidin-3- yl)-2-oxo-1,3- benzodiazol-5- yl]propoxy]propan- al | 388.20 | (400 MHz, DMSO-d₆) δ 9.68 (t, J = 2.0 Hz, 1H), 7.06-6.98 (m, 2H), 6.90-6.82 (m, 1H), 5.41 (m, 1H), 3.70 (t, J = 6.0 Hz, 2H), 3.39 (t, J = 6.4 Hz, 2H), 3.34 (s, 3H), 3.04 (s, 3H), 3.00-2.91 (m, 1H), 2.83-2.68 (m, 2H), 2.65-2.40 (m, 4H), 2.06-1.97 (m, 1H), 1.86-1.75 (m, 2H) |
| S14 | 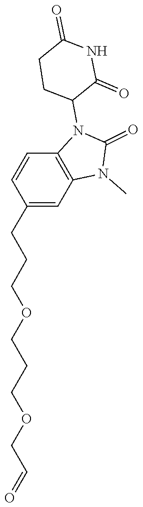 [SMILES: Cn1c(=O)n(C2CCC(=O)NC2=O)c2ccc(CCCOCCCOCC=O)cc21] | 2-(3-[3-[1-(2,6- dioxopiperidin-3- yl)-3-methyl-2- oxo-2,3-dihydro- 1H-1,3- benzodiazol-5- yl]propoxy]propoxy) acetaldehyde | 418.30 | (400 MHz, DMSO-d₆) δ 11.09 (s, 1H), 9.58 (s, 1H), 7.07-6.96 (m, 2H), 6.90- 6.80 (m, 1H), 5.40-5.30 (m, 1H), 4.16 (s, 2H), 3.58-3.33 (m, 9H), 2.97-2.84 (m, 1H), 2.78-2.53 (m, 4H), 2.10-2.00 (m, 1H), 1.85-1.70 (m, 4H) |
| S20 | 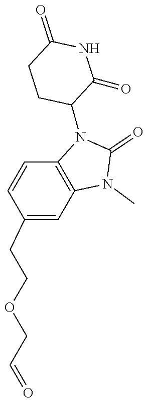 [SMILES: Cn1c(=O)n(C2CCC(=O)NC2=O)c2ccc(CCOCC=O)cc21] | 2-[2-[1-(2,6- dioxopiperidin-3- yl)-3-methyl-2- oxo-2,3-dihydro- 1H-1,3- benzodiazol-5- yl]ethoxy]acetalde- hyde | 346.15 | (400 MHz, CD₃OD) δ 7.14 (d, J = 6.0 Hz, 1H), 7.02 (d, J = 2.1 Hz, 2H), 5.37- 5.27 (m, 1H), 4.64 (t, J = 5.0 Hz, 1H), 3.74 (t, J = 6.9 Hz, 2H), 3.52-3.36 (m, 5H), 3.02-2.85 (m, 3H), 2.83 (d, J = 4.3 Hz, 1H), 2.82-2.73 (m, 1H), 2.22-2.12 (m, 1H). |
| S21 | 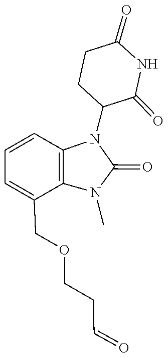 | 3-[[1-(2,6- dioxopiperidin-3- yl)-3-methyl-2- oxo-1,3- benzodiazol-4- yl]methoxy]propan- al | 346.10 | (400 MHz, DMSO-d₆) δ 11.10 (s, 1H), 9.65 (d, J = 2.3 Hz, 1H), 7.13 (d, J = 7.1 Hz, 1H), 7.01 (d, J = 7.0 Hz, 2H), 5.40- 5.30 (m, 1H), 4.75-4.60 (m, 2H), 3.56 (d, J = 9.6 Hz, 5H), 2.96-2.83 (m, 1H), 2.75-2.60 (m, 4H), 2.06-1.98 (m, 1H) |
| S23 | 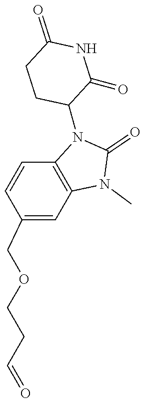 [SMILES: Cn1c(=O)n(C2CCC(=O)NC2=O)c2ccc(COCCC=O)cc21] | 3-[[1-(2,6- dioxopiperidin-3- yl)-3-methyl-2- oxo-1,3- benzodiazol-5- yl]methoxy]propan- al | 346.25 | (400 MHz, DMSO-d₆) δ 11.10 (s, 1H), 9.67 (t, J = 2.0 Hz, 1H), 7.17-7.10 (m, 1H), 7.08 (t, J = 7.3 Hz, 1H), 7.05-6.95 (m, 1H), 5.40-5.30 (m, 1H), 4.48 (d, J = 16.3 Hz, 2H), 3.75 (t, J = 6.0 Hz, 1H), 3.55-3.40 (m, 1H), 3.35 (s, 3H), 2.98- 2.84 (m, 1H), 2.78-2.68 (m, 1H), 2.67- 2.50 (m, 2H), 2.07-1.97 (m, 1H), 1.78- 1.65 (m, 1H) |
| S24 | 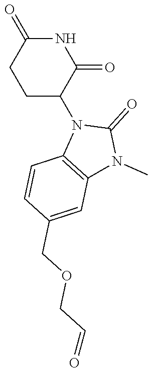 [SMILES: Cn1c(=O)n(C2CCC(=O)NC2=O)c2ccc(COCC=O)cc21] | 2-[[1-(2,6- dioxopiperidin-3- yl)-3-methyl-2- oxo-1,3- benzodiazol-5- yl]methoxy]acetal- dehyde | 332.25 | (400 MHz, DMSO-d₆) δ 11.10 (s, 1H), 9.67 (t, J = 2.0 Hz, 1H), 7.23-6.98 (m, 3H), 5.40-5.30 (m, 1H), 4.59 (s, 1H), 4.53 (d, J = 3.4 Hz, 1H), 3.35 (s, 3H), 3.29 (d, J = 5.4 Hz, 2H), 2.00-1.90 (m, 1H), 2.80-2.70 (m, 1H), 2.68-2.59 (m, 1H), 2.06-1.98 (m, 1H) |
| S29 | 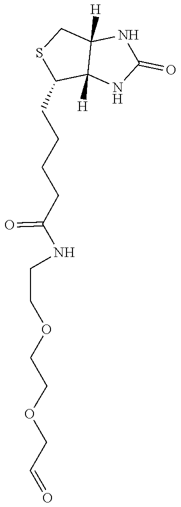 [SMILES: O=CCOCCOCCNC(=O)CCCC[C@@H]1SC[C@@H]2NC(=O)N[C@@H]21] | 5-[(3aS,4S,6aR)- 2-oxo-hexahydro- 1H-thieno[3,4- d]imidazol-4-yl]- N-[2-[2-(2- oxoethoxy)ethoxy] ethyl]pentanamide | 374.30 | (400 MHz, DMSO-d₆) δ 7.83 (s, 1H), 6.41 (d, J = 26.9 Hz, 1H), 5.87 (d, J = 4.9 Hz, 1H), 5.32-5.24 (m, 1H), 4.64- 4.30 (m, 3H), 4.24-4.04 (m, 3H), 2.87- 2.76 (m, 2H), 2.58 (d, J = 12.5 Hz, 1H), 2.07 (t, J = 7.4 Hz, 4H), 1.63 (s, 2H), 1.40 (d, J = 4.3 Hz, 1H), 1.33-1.23 (m, 4H) |
| S30 | 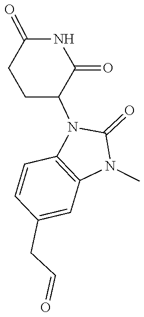 [SMILES: Cn1c(=O)n(C2CCC(=O)NC2=O)c2ccc(CC=O)cc21] | 2-[1-(2,6- dioxopiperidin-3- yl)-3-methyl-2- oxo-2,3-dihydro- 1H-1,3- benzodiazol-5- yl]acetaldehyde | 302.30 | (400 MHz, DMSO-d₆) δ 11.18 (s, 1H), 9.69 (d, J = 2.1 Hz, 1H), 7.71 (d, J = 2.7 Hz, 1H), 7.12 (s, 1H), 6.93 (s, 1H), 5.52- 5.47 (m, 1H), 3.42 (s, 3H), 2.98-2.84 (m, 1H), 2.80-2.70 (m, 1H), 2.68-2.59 (m, 1H), 2.06-1.98 (m, 1H) |
| S31 | 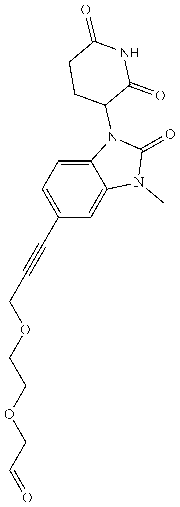 [SMILES: Cn1c(=O)n(C2CCC(=O)NC2=O)c2ccc(C#CCOCCOCC=O)cc21] | 2-[2-([3-[1-(2,6- dioxopiperidin-3- yl)-3-methyl-2- oxo-2,3-dihydro- 1H-1,3- benzodiazol-5- yl]prop-2-yn-1- yl]oxy)ethoxy]acet- aldehyde | 400.25 | (400 MHz, DMSO-d₆) δ 11.14 (s, 1H), 9.60 (s, 1H), 7.35 (d, J = 1.4 Hz, 1H), 7.22-7.11 (m, 2H), 5.40 (dd, J = 12.7, 5.4 Hz, 1H), 4.41 (d, J = 6.7 Hz, 2H), 4.22 (s, 2H), 3.68 (s, 4H), 3.35 (s, 3H), 2.95-2.83 (m, 1H), 2.76-2.60 (m, 2H), 2.07-1.99 (m, 1H). |
| S32 | 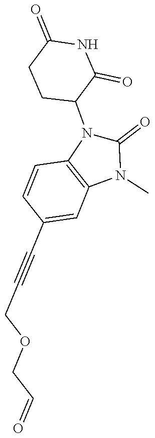 [SMILES: Cn1c(=O)n(C2CCC(=O)NC2=O)c2ccc(C#CCOCC=O)cc21] | 2-([3-[1-(2,6- dioxopiperidin-3- yl)-3-methyl-2- oxo2,3-dihydro- 1H-1,3- benzodiazol-5- yl]prop-2-yn-1- yl]oxy)acetaldehyde | 356.25 | (400 MHz, DMSO-d₆) δ 11.14 (s, 1H), 9.60 (s, 1H), 7.34 (s, 1H), 72.1-7.11 (m, 2H), 5.86 (d, J = 6.5 Hz, 2H), 5.40 (dd, J = 12.7, 5.3 Hz, 1H), 4.41 (s, 2H), 3.35 (s, 3H), 2.89 (t, J = 15.2 Hz, 1H), 2.77- 2.67 (m, 1H), 2.65 (s, 1H), 2.07-2.00 (m, 1H). |
| S15 | 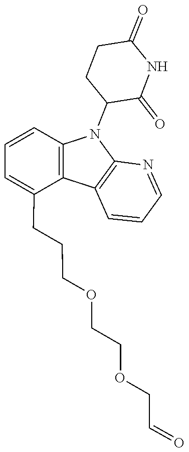 [SMILES: O=CCOCCOCCCc1cccc2c1c1cccnc1n2C1CCC(=O)NC1=O] | 2-(2-[3-[9-(2,6 dioxopiperidin-3- yl)pyrido[2,3- b]indol-5- yl]propoxy]ethoxy) acetaldehyde | 424.30 | N/A |
| S16 | 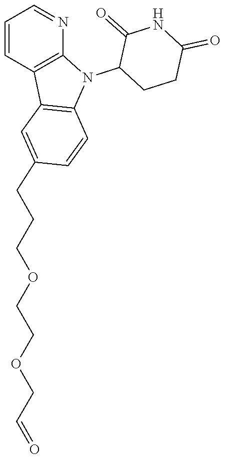 [SMILES: O=CCOCCOCCCc1ccc2c(c1)c1cccnc1n2C1CCC(=O)NC1=O] | 2-(2-[3-[9-(2,6- dioxopiperidin-3- yl)-9H-pyrido[2,3- b]indol-6- yl]propoxy]ethoxy) acetaldehyde | 424.30 | N/A |
| S17 | 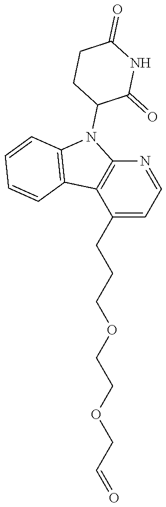 [SMILES: O=CCOCCOCCCc1ccnc2c1c1ccccc1n2C1CCC(=O)NC1=O] | 2-(2-[3-[9-(2,6- dioxopiperidin-3- yl)-9H-pyrido[2,3- b]indol-4- yl]propoxy]ethoxy) acetaldehyde | 424.30 | N/A |
| S18 | 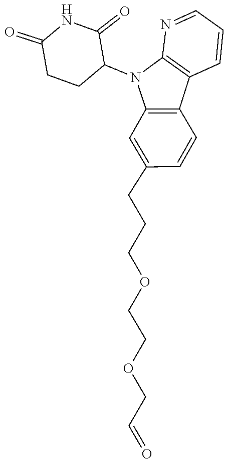 [SMILES: O=CCOCCOCCCc1ccc2c3cccnc3n(C3CCC(=O)NC3=O)c2c1] | 2-(2-[3-[9-(2,6- dioxopiperidin-3- yl)-9H-pyrido[2,3- b]indol-7- yl]propoxy]ethoxy) acetaldehyde | 424.30 | N/A |
| S19 | 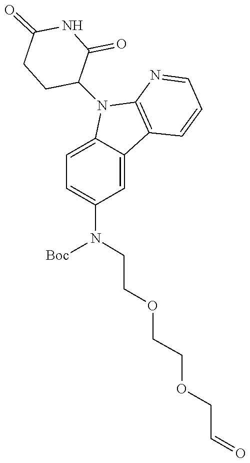 [SMILES: CC(C)(C)OC(=O)N(CCOCCOCC=O)c1ccc2c(c1)c1cccnc1n2C1CCC(=O)NC1=O] | tert-butyl (9-(2,6- dioxopiperidin-3- yl)-9H-pyrido[2,3- b]indol-6-yl)(2-(2- (2- oxoethoxy)ethoxy) ethyl)carbamate | 525.30 | N/A |
| S22 | 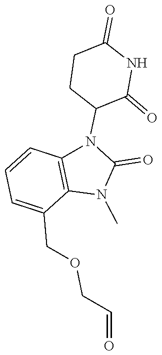 [SMILES: Cn1c(=O)n(C2CCC(=O)NC2=O)c2cccc(COCC=O)c21] | 2-[[1-(2,6- dioxopiperidin-3- yl)-3-methyl-2- oxo-1,3- benzodiazol-4- yl]methoxy]acetal- dehyde | 332.20 | N/A |
| S25 | 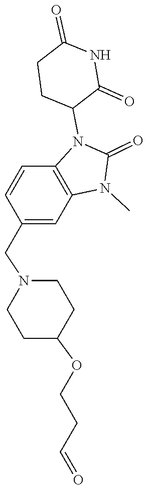 [SMILES: Cn1c(=O)n(C2CCC(=O)NC2=O)c2ccc(CN3CCC(OCCC=O)CC3)cc21] | 3-[(1-[[1-(2,6- dioxopiperidin-3- yl)-3-methyl-2- oxo-1,3- benzodiazol-5- yl]methyl]piperidin- 4- yl)oxy]propanal | 429.20 | N/A |
| S26 | 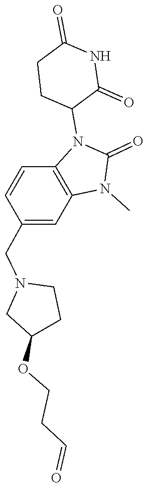 [SMILES: Cn1c(=O)n(C2CCC(=O)NC2=O)c2ccc(CN3CC[C@@H](OCCC=O)C3)cc21] | 3-[[(3R)-1-[[1- (2,6- dioxopiperidin-3- yl)-3-methyl-2- oxo-1,3- benzodiazol-5- yl]methyl]pyrrolidin- 3- yl]oxy]propanal | 415.15 | N/A |
| S27 | 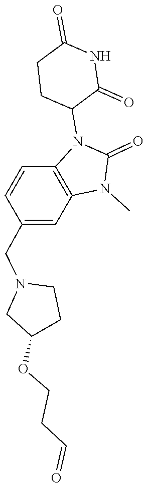 [SMILES: Cn1c(=O)n(C2CCC(=O)NC2=O)c2ccc(CN3CC[C@H](OCCC=O)C3)cc21] | 3-[[(3S)-1-[[1- (2,6- dioxopiperidin-3- yl)-3-methyl-2- oxo-1,3- benzodiazol-5- yl]methyl]pyrrolidin- 3- yl]oxy]propanal | 415.40 | N/A |
| S28 | 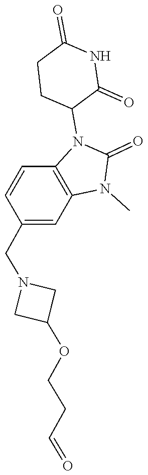 [SMILES: Cn1c(=O)n(C2CCC(=O)NC2=O)c2ccc(CN3CC(OCCC=O)C3)cc21] | 3-[(1-[[1-(2,6- dioxopiperidin-3- yl)-3-methyl-2- oxo-1,3- benzodiazol-5- yl]methyl]azetidin- 3-yl)oxy]propanal | 401.15 | N/A |

### INTERMEDIATE S33: 3-[3-methyl-2-oxo-5-[2-(3-oxocyclobutoxy)ethyl]-1,3-benzodiazol-1-yl]piperidine-2,6-dione

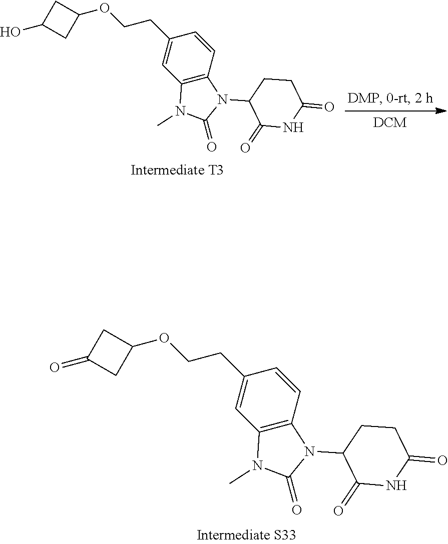 [SMILES: Cn1c(=O)n(C2CCC(=O)NC2=O)c2ccc(CCOC3CC(O)C3)cc21 ; Cn1c(=O)n(C2CCC(=O)NC2=O)c2ccc(CCOC3CC(=O)C3)cc21]

To a stirred solution of 3-[5-[2-(3-hydroxycyclobutoxy)ethyl]-3-methyl-2-oxo-1,3-benzodiazol-1-yl]piperidine-2,6-dione (300 mg, 0.80 mmol) in DCM (10.0 mL) was added Dess-Martin reagent (409 mg, 0.96 mmol) in portions at 0° C. under nitrogen atmosphere. The solution was stirred for 2 h at room temperature. Upon completion, the resulting solution was concentrated under reduced pressure and the residue was purified by reversed phase flash chromatography with the following conditions: Column: WelFlash™ C18-I, 20-40 um, 330 g; Eluent A: water (plus 10 mmol/L FA); Eluent B: ACN; Gradient: 20%-40% B in 20 min; Flow rate: 80 mL/min; Detector: UV 220/254 nm; desired fractions were collected at 35% B and concentrated under reduced pressure to afford 3-[3-methyl-2-oxo-5-[2-(3-oxocyclobutoxy)ethyl]-1,3-benzodiazol-1-yl]piperidine-2,6-dione (220 mg, 74%) as an off white solid. ¹H NMR (400 MHz, DMSO-d₆) δ 11.08 (s, 1H), 7.11 (d, J=1.5 Hz, 1H), 7.02 (d, J=8.0 Hz, 1H), 7.00-6.90 (m, 1H), 5.40-5.30 (m, 1H), 4.35-4.20 (m, 1H), 3.63 (t, J=7.0 Hz, 2H), 3.33 (s, 3H), 3.27-3.19 (m, 2H), 3.01-2.84 (m, 5H), 2.74-2.59 (m, 2H), 2.05-1.90 (m, 1H). LC/MS (ESI, m/z): [(M+1)]⁺=372.30

### INTERMEDIATE X1: (2S,4R)—N-[[2-(4-bromobutoxy)-4-(4-methyl-1,3-thiazol-5-yl)phenyl]methyl]-1-[(2S)-2-[(1-fluorocyclopropyl)formamido]-3,3-dimethylbutanoyl]-4-hydroxypyrrolidine-2-carboxamide

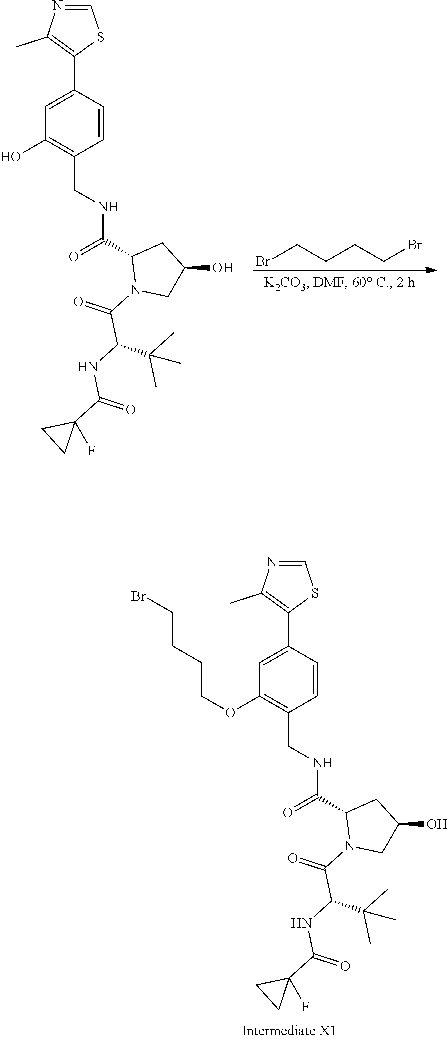 [SMILES: Cc1ncsc1-c1ccc(CNC(=O)[C@@H]2C[C@@H](O)CN2C(=O)[C@@H](NC(=O)C2(F)CC2)C(C)(C)C)c(O)c1 ; BrCCCCBr ; Cc1ncsc1-c1ccc(CNC(=O)[C@@H]2C[C@@H](O)CN2C(=O)[C@@H](NC(=O)C2(F)CC2)C(C)(C)C)c(OCCCCBr)c1]

To a stirred solution of (2S,4R)-1-[(2S)-2-[(1-fluorocyclopropyl)formamido]-3,3-dimethylbutanoyl]-4-hydroxy-N-[[2-hydroxy-4-(4-methyl-1,3-thiazol-5-yl)phenyl]methyl]pyrrolidine-2-carboxamide (50.0 mg, 0.10 mmol) and Cs₂CO₃ (61.2 mg, 0.20 mmol) in DMF (3.00 mL) were added 1,4-dibromobutane (24.3 mg, 0.11 mmol) at 60° C. The resulting mixture was stirred for 2 h at 60° C. The resulting solution was cooled and purified by reversed phase flash chromatography with the following conditions: Column: Spherical C¹⁸ Column, 20-40 um, 120 g; Mobile Phase A: water (plus 0.05% NH₄HCO₃), Mobile Phase B: ACN; Flow rate: 60 mL/min; Gradient: 30% B to 50% B in 25 min, detector: UV 220/254 nm). The fractions containing the desired product were collected at 40% B and concentrated under reduced pressure to afford (2S,4R)—N-[[2-(4-bromobutoxy)-4-(4-methyl-1,3-thiazol-5-yl)phenyl]methyl]-1-[(2S)-2-[(1-fluorocyclopropyl)formamido]-3,3-dimethylbutanoyl]-4-hydroxypyrrolidine-2-carboxamide (59.0 mg, 94%) as a white solid. ¹H NMR (400 MHz, CD₃OD) δ 8.89 (d, J=2.7 Hz, 1H), 7.55-7.45 (m, 1H), 7.04 (d, J=6.2 Hz, 2H), 4.76 (d, J=5.3 Hz, 1H), 4.70-4.60 (m, 1H), 4.52 (d, J=6.1 Hz, 1H), 4.47 (s, 1H), 4.43 (d, J=4.7 Hz, 1H), 4.14 (t, J=5.9 Hz, 2H), 3.91-3.79 (m, 2H), 3.58 (t, J=6.4 Hz, 2H), 3.37 (s, 2H), 2.51 (d, J=3.2 Hz, 3H), 2.31-2.21 (m, 1H), 2.12 (d, J=7.1 Hz, 3H), 2.10-1.95 (m, 2H), 1.39 (d, J=9.4 Hz, 1H), 1.35-1.20 (m, 3H), 1.05 (s, 9H). LC/MS (ESI, m/z): [(M+1)]⁺=667.40, 669.40.

The following intermediates in Table 39 were synthesized according to the above procedure:

TABLE 39

| Inter- | | | | |
|---|---|---|---|---|
| medi- | | | MS: | |
| ate | Structure | Name | [(M + 1)]⁺ | ¹H NMR |
| X3 | 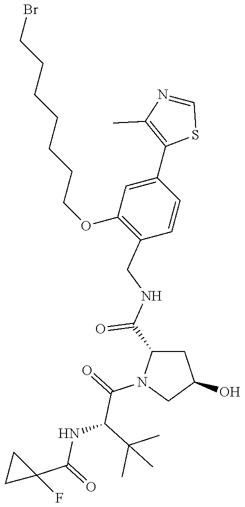 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCCBr)c1] | (2S,4R)-N-([2-[(7- bromoheptyl)oxy]- 4-(4-methyl-1,3- thiazol-5- yl)phenyl]methyl)- 1-[(2S)-2-[(1- fluorocyclopropyl)for- mamido]-3,3- dimethylbutanoyl]- 4- hydroxypyrrolidine- 2-carboxamide | 709.50, 711.50. | (400 MHz, CDCl₃) δ 8.74 (s, 1H), 7.35 (d, J = 7.7 Hz, 1H), 7.26 (d, J = 6.1 Hz, 1H), 7.10-7.00 (m, 1H), 6.98-6.90 (m, 1H), 6.88 (d, J = 1.6 Hz, 1H), 4.76 (t, J = 7.7 Hz, 1H), 4.61-4.40 (m, 4H), 4.10-3.97 (m, 3H), 3.70-3.60 (m, 1H), 3.44 (t, J = 6.8 Hz, 2H), 2.82 (s, 1H), 2.70- 2.60 (m, 1H), 2.56 (s, 3H), 2.15- 2.01 (m, 1H), 1.96-1.83 (m, 4H), 1.68-1.40 (m, 3H), 1.40-1.24 (m, 4H), 0.96 (s, 9H) |
| X5 | 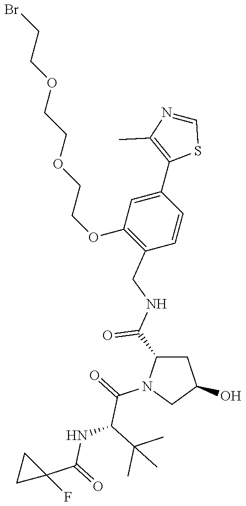 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCBr)c1] | (2S,4R)-N-[(2-[2- [2-(2- bromoethoxy)ethoxy] ethoxy]-4-(4- methyl-1,3-thiazol- 5- yl)phenyl)methyl]- 1-[(2S)-2-[(1- fluorocyclopropyl)for- mamido]-3,3- dimethylbutanoyl]- 4- hydroxypyrrolidine- 2-carboxamide | 727.50, 729.50. | (400 MHz, CDCl3) δ 8.71 (d, J = 2.5 Hz, 1H), 7.38-7.28 (m, 2H), 7.15-7.06 (m, 1H), 7.02-6.96 (m, 1H), 6.94-6.88 (m, 1H), 4.70-4.60 (m, 1H), 4.57-4.40 (m, 4H), 4.30- 4.13 (m, 2H), 4.03-3.95 (m, 3H), 3.93-3.78 (m, 2H), 3.82-3.76 (m, 1H), 3.76-3.70 (m, 1H), 3.68 (m, 1H), 3.48 (t, J = 6.2 Hz, 1H), 2.54 (d, J = 3.4 Hz, 3H), 2.50-2.40 (m, 1H), 2.17-2.07 (m, 1H), 2.02 (s, 1H), 1.41-1.14 (m, 3H), 1.30 (s, 1H), 0.98 (s, 9H) |
| X6 | 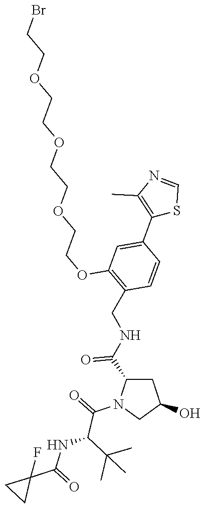 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCOCCOCCBr)c1] | (2S,4R)-N-[[2-(2- [2-[2-(2- bromoethoxy)ethoxy] ethoxy]ethoxy)- 4-(4-methyl-1,3- thiazol-5- yl)phenyl]methyl]- 1-[(2S)-2-[(1- fluorocyclopropyl)for- mamido]-3,3- dimethylbutanoyl]- 4- hydroxypyrrolidine- 2-carboxamide | 771.60, 773.60. | (400 MHz, CDCl₃) δ 8.72 (s, 1H), 7.39-7.28 (m, 2H), 7.15-7.05 (m, 1H), 7.00-6.96 (m, 1H), 6.94-6.88 (m, 1H), 4.69 (t, J = 7.9 Hz, 1H), 4.65-4.42 (m, 5H), 4.30-4.14 (m, 2H), 4.03-3.86 (m, 3H), 3.85-3.69 (m, 6H), 3.68 (s, 4H), 3.71-3.63 (m, 1H), 3.50-3.40 (m, 2H), 2.87 (s, 1H), 2.55 (d, J = 4.2 Hz, 4H), 2.19-2.04 (m, 2H), 1.33-1.23 (m, 3H), 0.98 (s, 9H) |
| X2 | 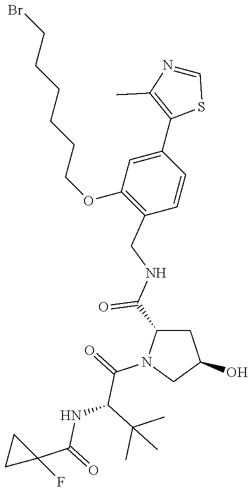 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCCCBr)c1] | (2S,4R)-N-([2-[(6- bromohexyl)oxy]- 4-(4-methyl-1,3- thiazol-5- yl)phenyl]methyl)- 1-[(2S)-2-[(1- fluorocyclopropyl)for- mamido]-3,3- dimethylbutanoyl]- 4- hydroxypyrrolidine- 2-carboxamide | 695.00, 697.00. | N/A |
| X4 | 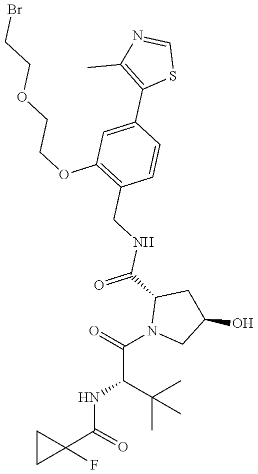 [SMILES: Cc1ncsc1-c1ccc(CNC(=O)[C@@H]2C[C@@H](O)CN2C(=O)[C@@H](NC(=O)C2(F)CC2)C(C)(C)C)c(OCCOCCBr)c1] | (2S,4R)-N-([2-[2- (2- bromoethoxy)ethoxy]- 4-(4-methyl-1,3- thiazol-5- yl)phenyl]methyl)- 1-[(2S)-2-[(1- fluorocyclopropyl)for- mamido]-3,3- dimethylbutanoyl]- 4- hydroxypyrrolidine- 2-carboxamide | 683.40, 685.40. | N/A |

### INTERMEDIATE X7: methyl 1-(5-[3-[5-(dimethylamino)naphthalene-1-sulfonamido]phenyl]-2-[[2-(2-iodoethoxy)ethyl]amino]pyrimidin-4-yl)-3-phenylpyrrolidine-2-carboxylate

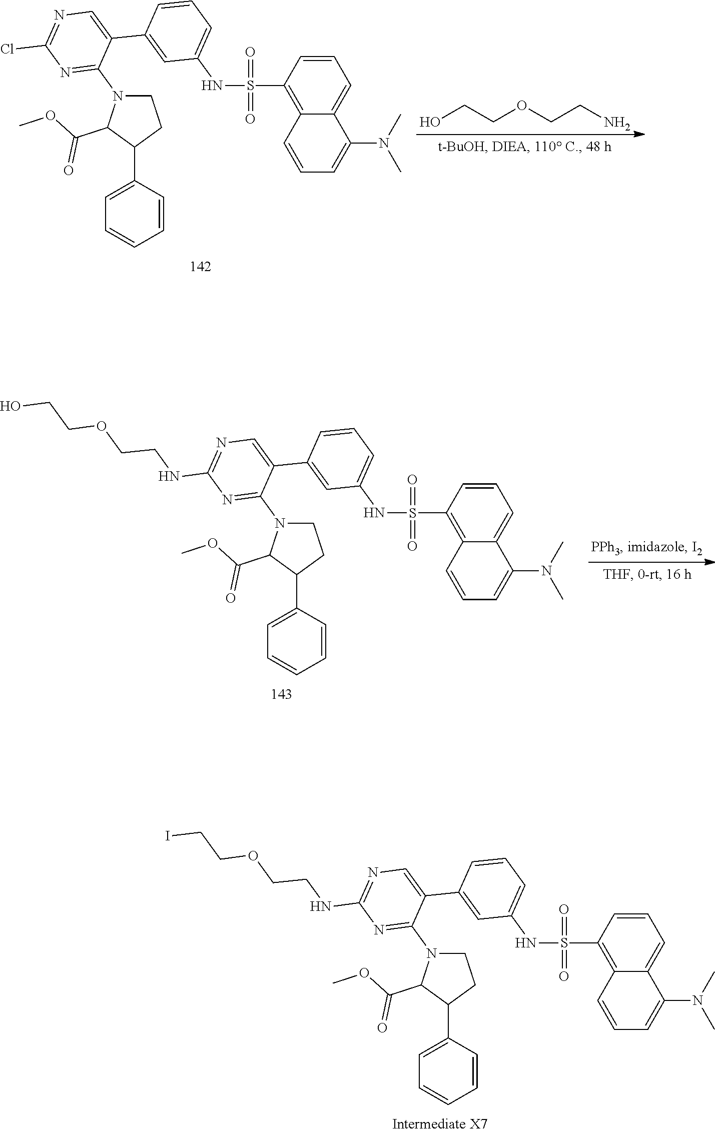

### Step 1: methyl 1-(5-[3-[5-(dimethylamino)naphthalene-1-sulfonamido]phenyl]-2-[[2-(2-hydroxyethoxy)ethyl]amino]pyrimidin-4-yl)-3-phenylpyrrolidine-2-carboxylate

To a stirred solution of methyl 1-(2-chloro-5-[3-[5-(dimethylamino)naphthalene-1-sulfonamido]phenyl]pyrimidin-4-yl)-3-phenylpyrrolidine-2-carboxylate (300 mg, 0.47 mmol) and 2-(2-aminoethoxy)ethanol (98.2 mg, 0.93 mmol) in t-BuOH (5.00 mL) was added DIEA (181 mg, 1.40 mmol) at room temperature under nitrogen atmosphere. The solution was sealed for 48 h at 110° C. Upon completion, the mixture was cooled down to room temperature and purified by reversed phase flash chromatography with the following conditions: Column: WelFlash™ C18-I, 20-40 um, 120 g; Eluent A: water (plus 10 mmol/L NH₄HCO₃); Eluent B: ACN; Gradient: 50%-75% B in 20 min; Flow rate: 55 mL/min; Detector: UV 220/254 nm; desired fractions were collected at 70% B and concentrated under reduced pressure to afford methyl 1-(5-[3-[5-(dimethylamino)naphthalene-1-sulfonamido]phenyl]-2-[[2-(2-hydroxyethoxy)ethyl]amino]pyrimidin-4-yl)-3-phenylpyrrolidine-2-carboxylate (300 mg, 90%) as a light yellow solid. ¹H NMR (400 MHz, DMSO-d₆) δ 10.69 (s, 1H), 8.42 (t, J=7.8 Hz, 2H), 8.25-8.10 (m, 1H), 7.75-7.65 (m, 2H), 7.53-7.14 (m, 8H), 7.04 (d, J=20.3 Hz, 1H), 6.97-6.53 (m, 2H), 4.67-4.41 (m, 2H), 3.53 (s, 1H), 3.50-3.37 (m, 7H), 3.30-3.20 (m, 4H), 3.09 (s, 2H), 2.80-2.60 (m, 7H). LC/MS (ESI, m/z): [(M+1)]⁺=711.20.

The following intermediates in Table 40 were synthesized according to the above procedure:

TABLE 40

| Inter- | | | | |
|---|---|---|---|---|
| medi- | | | MS: | |
| ate | Structure | Name | [(M + 1)]⁺ | ¹H NMR |
| 143- 1 | 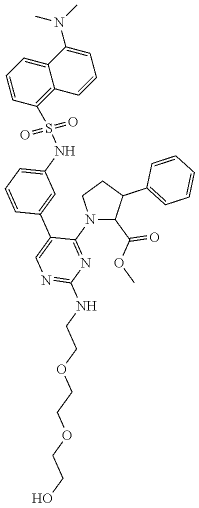 [SMILES: COC(=O)C1C(c2ccccc2)CCN1c1nc(NCCOCCOCCO)ncc1-c1cccc(NS(=O)(=O)c2cccc3c(N(C)C)cccc23)c1] | methyl 1-(5-[3-[5- (dimethylamino)naph- thalene-1- sulfonamido]phen- yl]-2-([2-[2-(2- hydroxyethoxy)eth- oxy]ethyl]amino)py- rimidin-4-yl)-3- phenylpyrrolidine- 2-carboxylate | 755.25 | (400 MHz, DMSO-d₆) δ 10.70 (s, 1H), 8.47-8.36 (m, 2H), 8.21 (d, J = 7.3 Hz, 1H), 7.70-7.60 (m, 2H), 7.45 (s, 1H), 7.38-7.23 (m, 4H), 7.19 (d, J = 7.3 Hz, 3H), 7.07 (s, 1H), 6.86 (s, 2H), 6.56 (s, 1H), 4.63 (d, J = 10.6 Hz, 2H), 3.50-3.30 (m, 11H), 3.24 (s, 4H), 3.09 (s, 3H), 2.80- 2.60 (m, 6H), 2.11 (s, 1H) |
| 143- 2 | 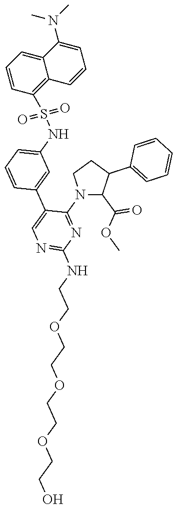 [SMILES: COC(=O)C1C(c2ccccc2)CCN1c1nc(NCCOCCOCCOCCO)ncc1-c1cccc(NS(=O)(=O)c2cccc3c(N(C)C)cccc23)c1] | methyl 1-(5-[3-[5- (dimethylamino)naph- thalene-1- sulfonamido]phen- yl]-2-(12-hydroxy- 4,7,10-trioxa-1- azadodecan-1- yl)pyrimidin-4-yl)- 3- phenylpyrrolidine- 2-carboxylate | 799.30 | (400 MHz, DMSO-d₆) δ 10.70 (s, 1H), 8.42 (t, J = 7.2 Hz, 2H), 8.21 (d, J = 7.3 Hz, 1H), 7.70- 7.60 (m, 2H), 7.46 (s, 1H), 7.37- 7.23 (m, 4H), 7.20-7.10 (m, 3H), 7.07 (s, 1H), 6.86 (s, 2H), 6.55 (s, 1H), 4.69-4.52 (m, 2H), 3.53 (s, 1H), 3.49 (d, J = 1.6 Hz, 11H), 3.50-3.30 (m, 4H), 3.24 (s, 2H), 3.09 (s, 2H), 2.80-2.60 (m, 6H), 2.11 (s, 1H) |

### Step 2: methyl 1-(5-[3-[5-(dimethylamino)naphthalene-1-sulfonamido]phenyl]-2[[2-(2-iodoethoxy)ethyl]amino]pyrimidin-4-yl)-3-phenylpyrrolidine-2-carboxylate

To a stirred solution of PPh₃ (221 mg, 0.84 mmol) and imidazole (144 mg, 2.11 mmol) in THF (5.00 mL) was added I₂ (161 mg, 0.63 mmol) over 1 min at 0° C. under nitrogen atmosphere. The solution was stirred for 30 min at 0° C. Then methyl 1-(5-[3-[5-(dimethylamino)naphthalene-1-sulfonamido]phenyl]-2-[[2-(2-hydroxyethoxy)ethyl]amino]pyrimidin-4-yl)-3-phenylpyrrolidine-2-carboxylate (300 mg, 0.42 mmol) in THF (5.00 mL) was added dropwise over 5 min at 0° C. The reaction was allowed to react for 16 h at room temperature. Upon completion, the solution was concentrated under reduced pressure and the residue was purified by silica gel column chromatography, eluted with DCM/MeOH (v:v=25/1) to afford methyl 1-(5-[3-[5-(dimethylamino)naphthalene-1-sulfonamido]phenyl]-2-[[2-(2-iodoethoxy)ethyl]amino]pyrimidin-4-yl)-3-phenylpyrrolidine-2-carboxylate (300 mg, 87%) as a light yellow solid. ¹H NMR (400 MHz, DMSO-d₆) δ 10.73 (s, 1H), 8.42 (t, J=8.3 Hz, 2H), 8.21 (d, J=7.2 Hz, 1H), 7.69-7.51 (m, 6H), 7.37-7.13 (m, 6H), 7.10-6.80 (m, 2H), 4.72-4.41 (m, 1H), 3.64 (t, J=6.4 Hz, 2H), 3.48 (s, 3H), 3.37-3.17 (m, 6H), 3.10 (s, 2H), 2.89-2.65 (m, 6H), 2.11 (s, 3H). LC/MS (ESI, m/z): [(M+1)]⁺=821.10.

The following intermediates in Table 41 were synthesized according to the above procedure:

TABLE 41

| Inter- | | | | |
|---|---|---|---|---|
| medi- | | | MS: | |
| ate | Structure | Name | [(M + 1)]⁺ | ¹H NMR |
| X8 | 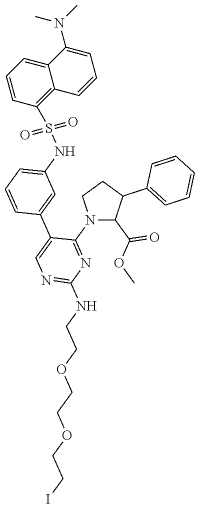 [SMILES: COC(=O)C1C(c2ccccc2)CCN1c1nc(NCCOCCOCCI)ncc1-c1cccc(NS(=O)(=O)c2cccc3c(N(C)C)cccc23)c1] | methyl 1-(5-[3-[5- (dimethylamino)naph- thalene-1- sulfonamido]phenyl]- 2-([2-[2-(2- iodoethoxy)ethoxy]eth- yl]amino)pyrimidin- 4-yl)-3- phenylpyrrolidine-2- carboxyate | 865.20 | (400 MHz, CD₃OD) δ 8.49 (d, J = 8.4 Hz, 2H), 8.25 (d, J = 7.3 Hz, 1H), 7.66-7.53 (m, 5H), 7.34 (t, J = 7.2 Hz, 2H), 7.30-7.15 (m, 5H), 6.97-6.86 (m, 1H), 3.80- 3.70 (m, 2H), 3.66-3.53 (m, 7H), 3.49-3.34 (m, 2H), 3.34-3.31 (m, 5H), 3.20 (s, 3H), 2.88-2.65 (m, 6H), 2.56 (s, 1H), 2.22 (s, 1H) |
| X9 | 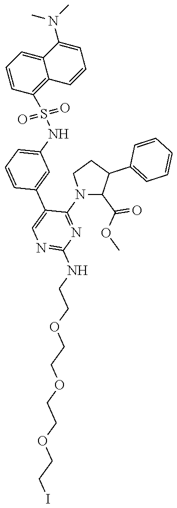 [SMILES: COC(=O)C1C(c2ccccc2)CCN1c1nc(NCCOCCOCCOCCI)ncc1-c1cccc(NS(=O)(=O)c2cccc3c(N(C)C)cccc23)c1] | methyl 1-(5-[3-[5- (dimethylamino)naph- thalene-1- sulfonamido]phenyl]- 2-(12-iodo-4,7,10- trioxa-1- azadodecan-1- yl)pyrimidin-4-yl)- 3-phenylpyrrolidine- 2-carboxamide | 909.35 | (400 MHz, CD₃OD) δ 8.49 (t, J = 8.4 Hz, 2H), 8.32-8.22 (m, 1H), 7.76-7.58 (m, 5H), 7.40- 7.12 (m, 7H), 7.04-6.78 (m, 1H), 3.78-3.49 (m, 13H), 3.49-3.31 (m, 2H), 3.31-3.21 (m, 7H), 3.20- (s, 2H), 2.91-2.67 (m, 6H), 2.57 (s, 1H), 2.26 (d, J = 7.5 Hz, 1H) |

### INTERMEDIATE Y1: 2-Amino-5-(1-[2-[2-(2-[3-[1-(2, 6-dioxopiperidin-3-yl)-3-methyl-2-oxo-1,3-benzodiazol-5-yl]propoxy]ethoxy)ethoxy]ethyl]pyrazol-4-yl)pyridine-3-carboxylic acid

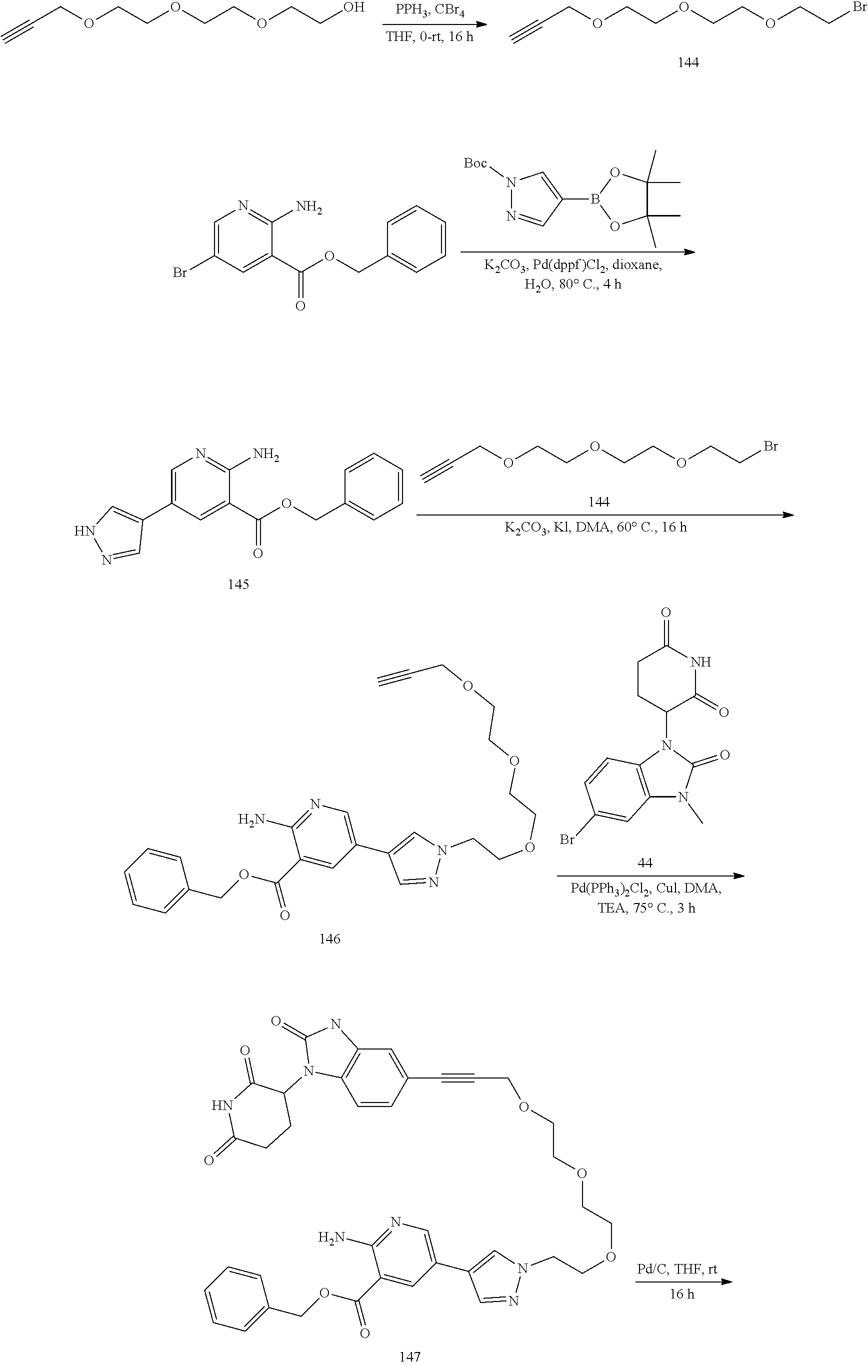

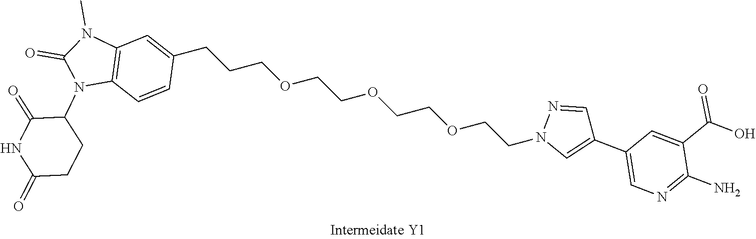 [SMILES: Cn1c(=O)n(C2CCC(=O)NC2=O)c2ccc(CCCOCCOCCOCCn3cc(-c4cnc(N)c(C(=O)O)c4)cn3)cc21]

### Step 1: 1-bromo-2-[2-[2-(prop-2-yn-1-yloxy)ethoxy]ethoxy]ethane

To a stirred solution of 2-[2-[2-(prop-2-yn-1-yloxy)ethoxy]ethoxy]ethanol (10.0 g, 53.1 mmol) in THF (100 mL) was added triphenylphosphine (27.9 g, 106 mmol) at 0° C. under nitrogen atmosphere followed by the addition of a solution of CBr₄ (35.2 g, 106 mmol) in THF (100 mL) over 20 min. The resulting mixture was stirred for 16 h at room temperature under nitrogen atmosphere. The resulting mixture was filtered. The filter cake was washed with THF (3×20.0 mL). The filtrate was concentrated under reduced pressure. The residue was purified by silica gel column chromatography, eluted with Petroleum ether/EtOAc (v:v=12:1) to afford 1-bromo-2-[2-[2-(prop-2-yn-1-yloxy)ethoxy]ethoxy]ethane (10.0 g, 75%) as a colorless oil. ¹H NMR (400 MHz, DMSO-d₆) δ 4.15 (d, J=2.4 Hz, 2H), 3.74 (t, J=5.8 Hz, 2H), 3.63-3.50 (m, 10H), 3.43 (t, J=2.4 Hz, 1H).

The following intermediates in Table 42 were synthesized according to the above procedure:

TABLE 42

| Intermediate | Structure | Name | ¹H NMR |
|---|---|---|---|
| 144-1 | 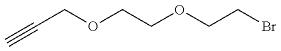 | 1-bromo-2-[2-(prop- 2-yl-1- yloxy)ethoxy]ethane | (400 MHz, DMSO-d₆) δ 4.15 (d, J = 2.4 Hz, 2H), 3.60-33.8 (m, 9H) |
| 144-2 | 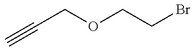 | 3-(2- bromoethoxy)prop-1- yne | (400 MHz, DMSO-d₆) δ 4.21 (d, J = 2.4 Hz, 2H), 3.80-3.70 (m, 2H), 3.68- 3.55 (m, 2H), 3.47 (d, J = 4.8 Hz, 1H) |

### Step 2: benzyl 2-amino-5-(1H-pyrazol-4-yl)pyridine-3-carboxylate

To a stirred solution of benzyl 2-amino-5-bromopyridine-3-carboxylate (34.5 g, 112 mmol) and tert-butyl 4-(4,4,5,5-tetramethyl-1,3 ,2-dioxaborolan-2-yl)pyrazole-1-carboxylate (49.6 g, 168 mmol) in 1,4-dioxane (900 mL) and water (300 mL) were added K₂CO₃ (46.6 g, 337 mmol) and Pd(dppf)Cl₂ CH₂Cl₂ (9.17 g, 11.3 mmol) at room temperature. The resulting mixture was purged with nitrogen three times and stirred for 4 h at 85° C. The mixture was cooled down to room temperature and concentrated under reduced pressure. The residue was diluted with water (500 mL) and extracted with EtOAc (3×400 mL). The combined organic layers were washed with brine (300 mL) and dried over anhydrous Na₂SO₄. After filtration, the filtrate was concentrated under reduced pressure. The residue was diluted with ACN (300 mL) and was stirred for 1 h at room temperature under nitrogen atmosphere. The precipitated solids were collected by filtration and washed with ACN (2×10.0 mL). The resulting solid was dried under vacuum to afford benzyl 2-amino-5-(1H-pyrazol-4-yl)pyridine-3-carboxylate (21 g, 63%) as a brown solid. ¹H NMR (400 MHz, DMSO-d₆) δ 12.92 (s, 1H), 8.54 (d, J=2.5 Hz, 1H), 8.23 (d, J=2.5 Hz, 1H), 8.02 (s, 2H), 7.49 (d, J=7.4 Hz, 2H), 7.41 (t, J=7.4 Hz, 2H), 7.35 (t, J=7.3 Hz, 1H), 7.14 (s, 2H), 5.37 (s, 2H). LC/MS (ESI, m/z): [(M+1)]⁺=295.15

### Step 3: benzyl 2-amino-5-[1-(2-[2-[2-(prop-2-yn-1-yloxy)ethoxy]ethoxy]ethyl)pyrazol-4-yl]pyridine-3-carboxylate

To a stirred solution of benzyl 2-amino-5-(1H-pyrazol-4-yl)pyridine-3-carboxylate (6.70 g, 22.77 mmol) and K₂CO₃ (9.44 g, 68.3 mmol) in DMA (50.0 mL) were added KI (1.89 g, 11.4 mmol) and 1-bromo-2-[2-[2-(prop-2-yn-1-yloxy)ethoxy]ethoxy]ethane (7.43 g, 29.6 mmol) at room temperature. The resulting mixture was stirred for 16 h at 60° C. under nitrogen atmosphere. After cooling down to room temperature, the mixture was purified by reversed phase flash chromatography with the following conditions: Column: WelFlash™ C18-I, 20-40 μm, 330 g; Eluent A: water (plus 10 mmol/L FA); Eluent B: ACN; Gradient: 40%-80% B in 20 min; Flow rate: 80 mL/min; Detector: UV 220/254 nm; desired fractions were collected at 70% B and concentrated under reduced pressure to afford benzyl 2-amino-5-[1-(2-[2-[2-(prop-2-yn-1-yloxy)ethoxy]ethoxy]ethyl)pyrazol-4-yl]pyridine-3-carboxylate (6.80 g, 64%) as a black oil. ¹H NMR (400 MHz, DMSO-d₆) δ 8.50 (d, J=2.5 Hz, 1H), 8.19 (d, J=2.5 Hz, 1H), 8.10 (s, 1H), 7.81 (s, 1H), 7.48 (d, J=7.4 Hz, 2H), 7.41 (t, J=7.4 Hz, 2H), 7.38-7.32 (m, 1H), 7.15 (s, 2H), 5.37 (s, 2H), 4.25 (t, J=5.4 Hz, 2H), 4.15 (d, J=2.4 Hz, 2H), 4.09 (d, J=2.4 Hz, 2H), 3.79 (t, J=5.3 Hz, 2H), 3.74 (t, J=5.8 Hz, 1H), 3.67 (t, J=6.4 Hz, 1H), 3.62-3.52 (m, 2H), 3.49 (d, J=13.8 Hz, 1H), 3.42 (d, J=2.5 Hz, 1H), 3.38 (d, J=2.4 Hz, 1H). LC/MS (ESI, m/z): [(M+1)]⁺=465.30.

The following intermediates were in Table 43 synthesized according to the above procedure:

TABLE 43

| Inter- | | | MS: | |
|---|---|---|---|---|
| mediate | Structure | Name | [(M + 1)]⁺ | ¹H NMR |
| 146-1 | 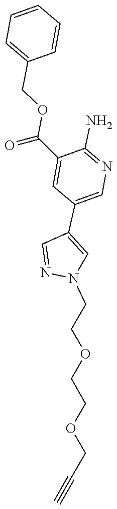 [SMILES: C#CCOCCOCCn1cc(-c2cnc(N)c(C(=O)OCc3ccccc3)c2)cn1] | benzyl 2-amino- 5-(1-[2-[2-(prop- 2-yn-1- yloxy)ethoxy]eth- yl]pyrazol-4- yl)pyridine-3- carboxylate | 421.25 | (400 MHz, DMSO-d₆) δ 8.50 (d, J = 2.4 Hz, 1H), 8.19 (d, J = 2.5 Hz, 1H), 8.11 (d, J = 0.9 Hz, 1H), 7.82 (d, J = 0.8 Hz, 1H), 7.51-7.46 (m, 2H), 7.45- 7.32 (m, 3H), 7.15 (s, 2H), 5.37 (s, 2H), 4.26 (t, J = 5.4 Hz, 2H), 4.10 (d, J = 2.5 Hz, 2H), 3.79 (t, J = 5.4 Hz, 2H), 3.53 (d, J = 8.2 Hz, 4H), 3.33 (s, 1H) |
| 146-2 | 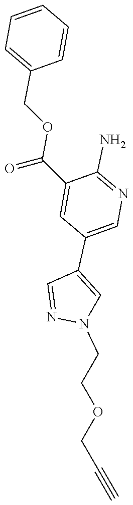 [SMILES: C#CCOCCn1cc(-c2cnc(N)c(C(=O)OCc3ccccc3)c2)cn1] | benzyl 2-amino- 5-[1-[2-(prop-2- yn-1- yloxy)ethyl]pyra- zol-4- yl]pyridine-3- carboxylate | 377.20 | (400 MHz, DMSO-d₆) δ 8.51 (s, 1H), 8.19 (d, J = 2.5 Hz, 1H), 8.10 (s, 1H), 7.83 (s, 1H), 7.48 (d, J = 7.3 Hz, 2H), 7.41 (t, J = 7.4 Hz, 2H), 7.38-7.31 (m, 1H), 7.15 (s, 2H), 5.37 (s, 2H), 4.28 (t, J = 5.3 Hz, 2H), 4.14 (d, J = 2.4 Hz, 2H), 3.83 (t, J = 5.3 Hz, 2H), 3.43 (t, J = 2.4 Hz, 1H) |
| 146-3 | 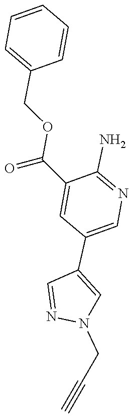 [SMILES: C#CCn1cc(-c2cnc(N)c(C(=O)OCc3ccccc3)c2)cn1] | benzyl 2-amino- 5-[2-[2-(prop-2- yn-1- yloxy)ethyl]pyra- zol-4- yl]pyridine-3- carboxylate | 333.25 | (400 MHz, DMSO-d₆) δ 8.52 (d, J = 2.5 Hz, 1H), 8.26-8.15 (m, 2H), 7.87 (s, 1H), 7.49 (d, J = 7.5 Hz, 2H), 7.45- 7.35 (m, 3H), 7.18 (s, 2H), 5.37 (s, 2H), 5.04 (d, J = 2.5 Hz, 2H), 3.50 (t, J = 2.5 Hz, 1H) |

### Step 4: benzyl 2-amino-5-[1-(2-[2-[2-([3-[1-(2,6-dioxopiperidin-3-yl)-3-methyl-2-oxo-1,3-benzodiazol-5-yl]prop-2-yn-1-yl]oxy)ethoxy]ethoxy]ethyl)pyrazol-4-yl]pyridine-3-carboxylate

To a stirred solution of 3-(5-bromo-3-methyl-2-oxo-1,3-benzodiazol-1-yl)piperidine-2,6-dione (400 mg, 1.18 mmol) and benzyl 2-amino-5-[1-(2-[2-[2-(prop-2-yn-1-yloxy)ethoxy]ethoxy]ethyl)pyrazol-4-yl]pyridine-3-carboxylate (2.20 g, 4.73 mmol) in DMSO (16.0 mL) and TEA (8.00 mL) were added CuI (22.5 mg, 0.12 mmol.) and Pd(PPh₃)₄ (137 mg, 0.12 mmol) at room temperature. The resulting mixture was purged with nitrogen three times and stirred for 3 h at 75° C. After cooling down to room temperature, the mixture was purified by reversed phase flash chromatography with the following conditions: Column: WelFlash™ C18-I, 20-40 μm, 330 g; Eluent A: water (plus 10 mmol/L NH₄HCO₃); Eluent B: ACN; Gradient: 50%-70% B in 25 min; Flow rate: 80 mL/min; Detector: UV 220/254 nm; desired fractions were collected at 60% B and concentrated under reduced pressure to afford benzyl 2-amino-5-[1-(2-[2-[2-([3-[1-(2,6-dioxopiperidin-3-yl)-3-methyl-2-oxo-1,3-benzodiazol-5-yl]prop-2-yn-1-yl]oxy)ethoxy]ethoxy]ethyl)pyrazol-4-yl]pyridine-3-carboxylate (400 mg, 47%) as a brown solid. ¹H NMR (400 MHz, DMSO-d₆) δ 11.10 (s, 1H), 8.54-8.44 (m, 1H), 8.25-8.14 (m, 1H), 8.10 (d, J=7.7 Hz, 1H), 7.81 (s, 1H), 7.70-7.60 (m, 1H), 7.58-7.50 (m, 1H), 7.48 (d, J=7.7 Hz, 2H), 7.45-7.30 (m, 3H), 7.28 (s, 1H), 7.13 (d, J=10.2 Hz, 2H), 5.36 (s, 2H), 5.31-5.19 (m, 1H), 4.34 (s, 1H), 4.30-4.20 (m, 3H), 3.87-3.69 (m, 3H), 3.59-3.39 (m, 11H), 2.79 (d, J=15.5 Hz, 1H), 2.68-2.50 (m, 1H), 2.10-1.95 (m, 1H). LC/MS (ESI, m/z): [(M+1)]⁺=722.20.

The following intermediates in Table 44 were synthesized according to the above procedure:

TABLE 44

| Interme- | | | MS: | |
|---|---|---|---|---|
| diate | Structure | Name | [(M + 1)]⁺ | ¹H NMR |
| 147-1 | 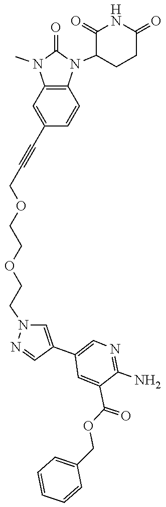 [SMILES: Cn1c(=O)n(C2CCC(=O)NC2=O)c2ccc(C#CCOCCOCCn3cc(-c4cnc(N)c(C(=O)OCc5ccccc5)c4)cn3)cc21] | benzyl 2-amino- 5-(1-[2-[2-([3-[1- (2,6- dioxopiperidin-3- yl)-3-methyl-2- oxo-1,3- benzodiazol-5- yl]prop-2-yn-1- yl]oxy)ethoxy]eth- yl]pyrazol-4- yl)pyridine-3- carboxylate | 678.30 | (400 MHz, DMSO-d₆) δ 11.12 (s, 1H), 8.50 (s, 1H), 8.18 (d, J = 2.4 Hz, 1H), 8.13 (s, 1H), 7.82 (s, 1H), 7.63 (m, 1H), 7.48 (d, J = 7.0 Hz, 2H), 7.44-7.37 (m, 2H), 7.37-7.31 (m, 1H), 7.26 (s, 1H), 7.12 (d, J = 7.4 Hz, 3H), 5.76 (s, 1H), 5.46-5.26 (m, 3H), 4.35 (s, 2H), 4.27 (m, 2H), 3.81 (t, J = 5.4 Hz, 2H), 3.60 (m, 4H), 2.96-2.82 (m, 1H), 2.77-2.59 (m, 2H), 2.55 (s, 1H), 2.11-1.95 (m, 1H), 1.49-1.19 (m, 1H) |
| 147-2 | 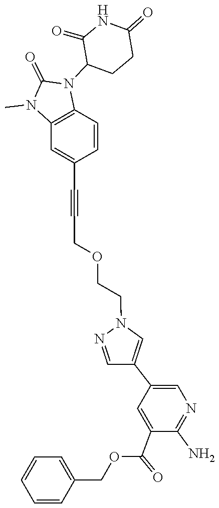 [SMILES: Cn1c(=O)n(C2CCC(=O)NC2=O)c2ccc(C#CCOCCn3cc(-c4cnc(N)c(C(=O)OCc5ccccc5)c4)cn3)cc21] | benzyl 2-amino- 5-[1-[2-([3-[1- (2,6- dioxopiperidin-3- yl)-3-methyl-2- oxo-1,3- benzodiazol-5- yl]prop-2-yn-1- yl]oxy)ethyl]pyra- zol-4-yl]pyridine- 3-carboxylate | 634.30 | (400 MHz, DMSO-d₆) δ 8.51 (s, 1H), 8.24-8.09 (m, 2H), 7.84 (s, 1H), 7.72-7.52 (m, 4H), 7.48 (d, J = 7.3 Hz, 2H), 7.14 (d, J = 6.2 Hz, 4H), 5.41 (d, J = 5.5 Hz, 1H), 5.36 (s, 2H), 4.40 (s, 2H), 4.33 (t, J = 5.3 Hz, 2H), 3.93 (t, J = 5.2 Hz, 2H), 3.33 (s, 3H), 2.88 (d, J = 15.7 Hz, 1H), 2.79-2.58 (m, 2H), 2.05-1.97 (m, 1H) |
| 147-3 | 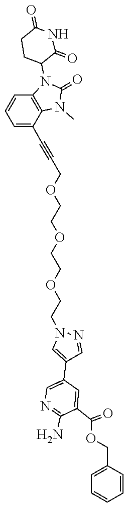 [SMILES: Cn1c(=O)n(C2CCC(=O)NC2=O)c2cccc(C#CCOCCOCCOCCn3cc(-c4cnc(N)c(C(=O)OCc5ccccc5)c4)cn3)c21] | benzyl 2-amino- 5-[1-(2-[2-[2-([3- [1-(2,6- dioxopiperidin-3- yl)-3-methyl-2- oxo-1,3- benzodiazol-4- yl]prop-2-yn-1- yl]oxy)ethoxy]eth- oxy]ethyl)pyrazol- 4-yl]pyridine-3- carboxylate | 772.40 | (400 MHz, DMSO-d₆) δ 11.12 (s, 1H), 8.48 (d, J = 2.4 Hz, 1H), 8.18 (d, J = 2.4 Hz, 1H), 8.10 (s, 1H), 7.80 (s, 1H), 7.47 (d, J = 7.3 Hz, 2H), 7.37 (m, 3H), 7.22-7.06 (m, 4H), 7.01 (t, J = 7.9 Hz, 1H), 5.36 (d, J = 8.3 Hz, 2H), 4.41 (s, 2H), 4.25 (t, J = 5.4 Hz, 2H), 3.79 (t, J = 5.4 Hz, 2H), 3.68-3.50 (m, 6H), 3.52 (d, J = 6.8 Hz, 6H), 2.87 (m, 1H), 2.78-2.59 (m, 2H), 2.06-1.99 (m, 1H) |
| 147-4 | 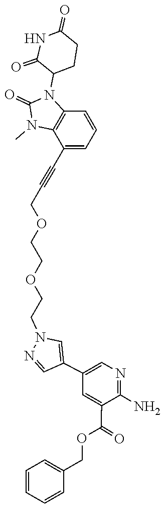 [SMILES: Cn1c(=O)n(C2CCC(=O)NC2=O)c2cccc(C#CCOCCOCCn3cc(-c4cnc(N)c(C(=O)OCc5ccccc5)c4)cn3)c21] | benzyl 2-amino- 5-(1-[2-[2-([3-[1- (2,6- dioxopiperidin-3- yl)-3-methyl-2- oxo-1,3- benzodiazol-4- yl]prop-2-yn-1- yl]oxy)ethoxy]eth- yl]pyrazol-4- yl)pyridine-3- carboxylate | 678.30 | (400 MHz, DMSO-d₆) δ 11.13 (s, 1H), 8.49 (d, J = 2.5 Hz, 1H), 8.17 (d, J = 2.5 Hz, 1H), 8.12 (s, 1H), 7.81 (s, 1H), 7.49-7.44 (m, 2H), 7.43-7.38 (m, 2H), 7.37-7.33 (m, 1H), 7.20- 7.09 (m, 3H), 7.08-7.03 (m, 1H), 6.99 (t, J = 7.8 Hz, 1H), 5.45-5.28 (m, 3H), 4.40 (s, 2H), 4.27 (t, J = 5.3 Hz, 2H), 3.81 (t, J = 5.4 Hz, 2H), 3.67- 3.59 (m, 3H), 3.58 (s, 3H), 2.99-2.81 (m, 1H), 2.79-2.55 (m, 2H), 2.08 (s, 2H) |
| 147-5 | 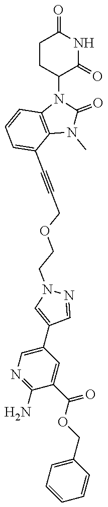 [SMILES: Cn1c(=O)n(C2CCC(=O)NC2=O)c2cccc(C#CCOCCn3cc(-c4cnc(N)c(C(=O)OCc5ccccc5)c4)cn3)c21] | benzyl 2-amino- 5-[1-[2-([3-[1- (2,6- dioxopiperidin-3- yl)-3-methyl-2- oxo-1,3- benzodiazol-4- yl]prop-2-yn-1- yl]oxy)ethyl]pyra- zol-4-yl]pyridine- 3-carboxylate | 634.20 | (400 MHz, DMSO-d6) δ 11.13 (s, 1H), 8.50 (d, J = 2.5 Hz, 1H), 8.22- 8.11 (m, 2H), 7.83 (s, 1H), 7.48 (m, 2H), 7.44-7.31 (m, 3H), 7.16 (d, J = 9.7 Hz, 3H), 7.10 (m, 1H), 7.00 (t, J = 7.9 Hz, 1H), 5.44-5.38 (m, 1H), 5.36 (s, 2H), 4.46 (s, 2H), 4.33 (t, J = 5.2 Hz, 2H), 3.94 (t, J = 5.3 Hz, 2H), 3.59 (s, 3H), 2.89 (t, J = 14.7 Hz, 1H), 2.76-2.59 (m, 2H), 2.11- 1.98 (m, 1H) |
| 147-6 | 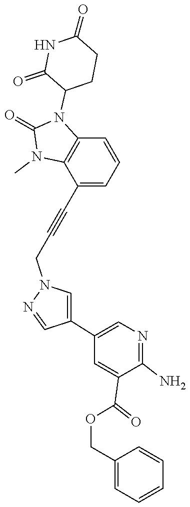 [SMILES: Cn1c(=O)n(C2CCC(=O)NC2=O)c2cccc(C#CCn3cc(-c4cnc(N)c(C(=O)OCc5ccccc5)c4)cn3)c21] | benzyl 2-amino- 5-(1-[3-[1-(2,6- dioxopiperidin-3- yl)-3-methyl-2- oxo-1,3- benzodiazol-4- yl]prop-2-yn-1- yl]pyrazol-4- yl)pyridine-3- carboxylate | 590.20 | (400 MHz, DMSO-d₆) δ 11.11 (s, 1H), 8.55 (s, 1H), 8.29 (s, 1H), 8.23 (d, J = 2.4 Hz, 1H), 7.92 (s, 1H), 7.67-7.53 (m, 2H), 7.49 (d, J = 7.0 Hz, 3H), 7.44-7.37 (m, 3H), 7.13 (s, 1H), 7.04 (d, J = 7.9 Hz, 1H), 5.37 (d, J = 2.4 Hz, 4H), 3.58 (s, 3H), 2.92-2.83 (m, 1H), 2.78-2.70 (m, 1H), 2.65 (t, J = 14.8 Hz, 2H), 2.06 1.99 (m, 1H) |

### Step 5: 2-Amino-5-(1-[2-[2-(2-[3-[1-(2,6-dioxopiperidin-3-yl)-3-methyl-2-oxo-1,3-benzodiazol-5-yl]propoxy]ethoxy)ethoxy]ethyl]pyrazol-4-yl)pyridine-3-carboxylic acid

To a stirred solution of benzyl 2-amino-5-[1-(2-[2-[2-([3-[1-(2,6-dioxopiperidin-3-yl)-3-methyl-2-oxo-1,3-benzodiazol-5-yl]prop-2-yn-1-yl ]oxy)ethoxy]ethoxy]ethyl)pyrazol-4-yl]pyridine-3-carboxylate (400 mg, 0.55 mmol) in THF (20.0 mL) was added 10% palladium on activated carbon (40.0 mg) at room temperature. The resulting mixture was purged with hydrogen three times and stirred for 16 h at room temperature under hydrogen atmosphere. The resulting mixture was concentrated under reduced pressure. The residue was diluted with DMF (6.00 mL) and stirred for 1 h at 75° C. under nitrogen atmosphere. The resulting mixture was filtered. The filter cake was washed with hot DMF (3×3.00 mL). The filtrate was purified by reversed phase flash chromatography with the following conditions: Column: WelFlash™ C18-I, 20-40 μm, 120 g; Eluent A: water (plus 10 mmol/L FA); Eluent B: ACN; Gradient: 20%-40% B in 25 min; Flow rate: 80 mL/min; Detector: UV 220/254 nm; desired fractions were collected at 30% B and concentrated under reduced pressure to afford 2-amino-5-(1-[2-[2-(2-[3-[1-(2,6-dioxopiperidin-3-yl)-3-methyl-2-oxo-1,3-benzodiazol-5-yl]propoxy]ethoxy)ethoxy]ethyl]pyrazol-4-yl)pyridine-3-carboxylic acid (150 mg, 43%) as a white solid. ¹H NMR (400 MHz, DMSO-d₆) δ 11.08 (s, 1H), 8.42 (d, J=2.5 Hz, 1H), 8.16 (d, J=2.6 Hz, 1H), 8.09 (s, 1H), 7.80 (s, 1H), 7.07-6.93 (m, 2H), 6.90-6.80 (m, 1H), 5.40-5.30 (m, 1H), 4.24 (t, J=5.4 Hz, 2H), 3.80 (t, J=5.4 Hz, 2H), 3.60-3.50 (m, 6H), 3.48-3.40 (m, 3H), 3.35 (d, J=6.4 Hz, 4H), 2.94-2.84 (m, 1H), 2.70-2.50 (m, 4H), 2.06-1.94 (m, 1H), 1.83-1.69 (m, 2H). LC/MS (ESI, m/z): [(M+1)]⁺=636.25.

The following intermediates in Table 45 were synthesized according to the above procedure:

TABLE 45

| Inter- | | | MS: | |
|---|---|---|---|---|
| mediate | Structure | Name | [(M + 1)]⁺ | ¹H NMR |
| Y2 | 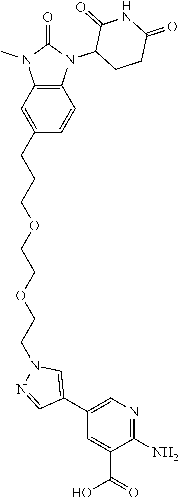 [SMILES: Cn1c(=O)n(C2CCC(=O)NC2=O)c2ccc(CCCOCCOCCn3cc(-c4cnc(N)c(C(=O)O)c4)cn3)cc21] | 2-Amino-5-[1-[2- (2-[3-[1-(2,6- dioxopiperidin-3- yl)-3-methyl-2- oxo-1,3- benzodiazol-5- yl]propoxy]ethoxy) ethyl]pyrazol-4- yl]pyridine-3- carboxylic acid | 592.30 | (400 MHz, DMSO-d₆) δ 11.07 (s, 1H), 8.41 (d, J = 2.5 Hz, 1H), 8.22- 8.04 (m, 2H), 7.81 (s, 1H), 6.97 (t, J = 4.0 Hz, 2H), 6.81 (d, J = 7.9 Hz, 1H), 5.32 (m, 1H), 4.26 (t, J = 5.5 Hz, 2H), 3.81 (t, J = 5.4 Hz, 2H), 3.60-3.45 (m, 4H), 3.36 (d, J = 12.9 Hz, 3H), 2.88 (d, J = 14.5 Hz, 1H), 2.78-2.54 (m, 4H), 2.05-1.95 (m, 1H), 1.77 (q, J = 7.1 Hz, 2H) |
| Y3 | 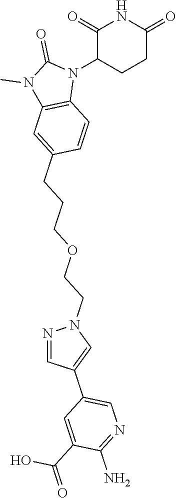 [SMILES: Cn1c(=O)n(C2CCC(=O)NC2=O)c2ccc(CCCOCCn3cc(-c4cnc(N)c(C(=O)O)c4)cn3)cc21] | 2-Amino-5-[1-(2- [3-[1-(2,6- dioxopiperidin-3- yl)-3-methyl-2- oxo-1,3- benzodiazol-5- yl]propoxy]ethyl) pyrazol-4- yl]pyridine-3- carboxylic acid | 548.30 | (400 MHz, DMSO-d₆) δ 11.06 (s, 1 H), 8.45 (d, J = 2.5 Hz, 1H), 8.19- 8.12 (m, 2H), 7.84 (s, 1H), 6.98- 6.91 (m, 2H), 6.85-6.70 (m, 1H), 5.40-5.30 (m, 1H), 4.27 (t, J = 5.3 Hz, 2H), 3.75 (t, J = 5.3 Hz, 2H), 3.38 (t, J = 6.3 Hz, 2H), 3.29 (s, 3H), 2.87 (d, J = 16.6 Hz, 1H), 2.76-2.62 (m, 1H), 2.57 (t, J = 7.8 Hz, 2H), 2.07 (s, 1H), 1.99 (d, J = 11.6 Hz, 1 H), 1.78 (q, J = 7.1 Hz, 2H) |
| Y4 | 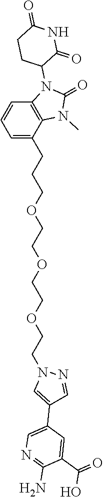 [SMILES: Cn1c(=O)n(C2CCC(=O)NC2=O)c2cccc(CCCOCCOCCOCCn3cc(-c4cnc(N)c(C(=O)O)c4)cn3)c21] | 2-Amino-5-(1-[2- [2-(2-[3-[1-(2,6- dioxopiperidin-3- yl)-3-methyl-2- oxo-1,3- benzodiazol-4- yl]propoxy]ethoxy) ethoxy]ethyl] pyrazol-4-yl) pyridine-3- carboxylic acid | 636.30 | (400 MHz, DMSO-d₆) δ 11.09 (s, 1 H), 8.47-8.33 (m, 1H), 8.19-8.13 (m, 1H), 8.08 (s, 1H), 7.79 (s, 1H), 6.99-6.91 (m, 2H), 6.88-6.81 (m, 1 H), 5.40-5.30 (m, 1H), 4.25 (t, J = 5.5 Hz, 2H), 3.80 (t, J = 5.4 Hz, 2 H), 3.54 (s, 3H), 3.53-3.44 (m, 7H), 3.41 (t, J = 6.1 Hz, 2H), 2.99-2.81 (m, 3H), 2.78-2.57 (m, 2H), 2.07- 1.93 (m, 1H), 1.86-1.70 (m, 2H) |
| Y6 | 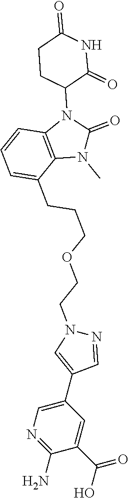 [SMILES: Cn1c(=O)n(C2CCC(=O)NC2=O)c2cccc(CCCOCCn3cc(-c4cnc(N)c(C(=O)O)c4)cn3)c21] | 2-Amino-5-[1-(2- [3-[1-(2,6- dioxopiperidin-3- yl)-3-methyl-2- oxo-1,3- benzodiazol-4- yl]propoxy]ethyl) pyrazol-4- yl]pyridine-3- carboxylic acid | 548.30 | (400 MHz, DMSO-d₆) δ 11.08 (s, 1 H), 8.44 (d, J = 2.5 Hz, 1H), 8.19- 8.11 (m, 2H), 7.84 (d, J = 0.8 Hz, 1 H), 6.98-6.86 (m, 2H), 6.80-6.70 (m, 1H), 5.40-5.30 (m, 1H), 4.28 (t, J = 5.3 Hz, 2H), 3.79 (t, J = 5.3 Hz, 2H), 3.48 (s, 3H), 3.44 (t, J = 6.0 Hz, 2H), 2.95-2.80 (m, 4H), 2.61 (d, J = 17.4 Hz, 1H), 2.03-1.94 (m, 1 H), 1.79 (p, J = 6.2 Hz, 2H) |
| Y7 | 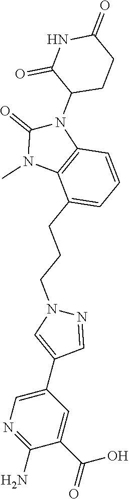 [SMILES: Cn1c(=O)n(C2CCC(=O)NC2=O)c2cccc(CCCn3cc(-c4cnc(N)c(C(=O)O)c4)cn3)c21] | 2-Amino-5-(1-[3- [1-(2,6- dioxopiperidin-3- yl)-3-methyl-2- oxo-1,3- benzodiazol-4- yl]propyl]pyrazol- 4-yl)pyridine-3- carboxylic acid | 504.30 | (400 MHz, DMSO-d₆) δ 11.08 (s, 1 H), 8.40 (d, J = 2.4 Hz, 1H), 8.19 (s, 2H), 7.82 (s, 1H), 6.98 (d, J = 4.6 Hz, 2H), 6.91 (q, J = 4.3 Hz, 1H), 5.40-5.30 (m, 1H), 4.21 (t, J = 6.8 Hz, 2H), 3.44 (s, 3H), 2.94-2.84 (m, 3H), 2.72 (d, J = 13.8 Hz, 1H), 2.62 (d, J = 18.3 Hz, 1H), 2.21-2.06 (m, 2H), 2.00-1.90 (m, 1H) |
| Y5 | 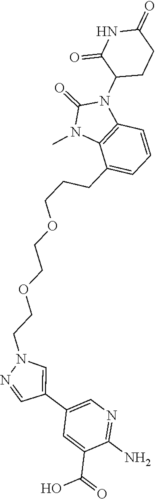 [SMILES: Cn1c(=O)n(C2CCC(=O)NC2=O)c2cccc(CCCOCCOCCn3cc(-c4cnc(N)c(C(=O)O)c4)cn3)c21] | | 592.30 | N/A |

### INTERMEDIATE Z1: 3-[5-(2-azidoethyl)-3-methyl-2-oxo-2,3-dihydro-1H-1,3-benzodiazol-1-yl]piperidine-2,6-dione

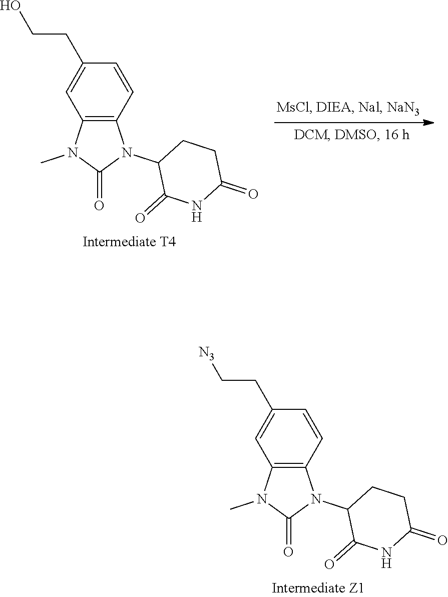 [SMILES: Cn1c(=O)n(C2CCC(=O)NC2=O)c2ccc(CCO)cc21 ; Cn1c(=O)n(C2CCC(=O)NC2=O)c2ccc(CCN=[N+]=[N-])cc21]

To a stirred solution of 3-[5-(2-hydroxyethyl)-3-methyl-2-oxo-2,3-dihydro-1H-1,3-benzodiazol-1-yl]piperidine-2,6-dione (120 mg, 0.40 mmol) and NaI (119 mg, 0.79 mmol) in DCM (6.00 mL) and DMSO (3.00 mL) was added DIEA (153 mg, 1.19 mmol) at room temperature under nitrogen atmosphere. To the above mixture was added MsCl (67.9 mg, 0.59 mmol) dropwise at 0° C. The resulting mixture was stirred for additional 4 h at room temperature. To the resulting mixture was added NaN₃ (129 mg, 1.98 mmol) and the resulting mixture was stirred for 12 h at room temperature. The reaction was quenched with water (2.00 mL). The resulting mixture was concentrated under reduced pressure. The residue was purified by reversed phase flash chromatography with the following conditions: Column: WelFlash™ C18-I, 20-40 μm, 120 g; Eluent A: water (plus 10 mmol/L HOAc); Eluent B: ACN; Gradient: 20%-45% B in 25 min; Flow rate: 80 mL/min; Detector: UV 220/254 nm; desired fractions were collected at 30% B and concentrated under reduced pressure to afford 3-[5-(2-azidoethyl)-3-methyl-2-oxo-2,3-dihydro-1H-1,3-benzodiazol-1-yl]piperidine-2,6-dione (70.0 mg, 54%) as a light yellow solid. ¹H NMR (400 MHz, DMSO-d₆) δ 11.10 (s, 1H), 7.14 (s, 1H), 7.05 (d, J=8.1 Hz, 1H), 6.99-6.92 (m, 1H), 5.40-5.30 (m, 1H), 3.34 (s, 3H), 3.59 (t, J=7.1 Hz, 2H), 3.21-3.12 (m, 1H), 2.89 (t, J=7.1 Hz, 2H), 2.80-2.70 (m, 1H), 2.64 (s, 1H), 2.01 (s, 1H). LC/MS (ESI, m/z): [(M+1)]⁺=329.10.

The following intermediate in Table 46 was synthesized according to the above procedure:

TABLE 46

| | | | MS: | |
|---|---|---|---|---|
| Intermediate | Structure | Name | [(M + 1)]⁺ | ¹H NMR |
| Z2 | 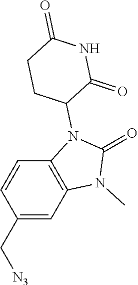 [SMILES: Cn1c(=O)n(C2CCC(=O)NC2=O)c2ccc(CN=[N+]=[N-])cc21] | 3-[5- (azidomethyl)-3- methyl-2-oxo- 1,3-benzodiazol- 1-yl]piperidine- 2,6-dione | 315.15 | (400 MHz, DMSO-d₆) δ 11.11 (s, 1H), 7.24 (d, J = 1.5 Hz, 1H), 7.16 (d, J = 8.1 Hz, 1H), 7.08 (m, 1H), 5.40-5.30 (m, 1H), 4.47 (s, 2H), 3.36 (s, 3H), 3.00-2.85 (m, 1H), 2.80-2.70 (m, 1H), 2.70- 2.60 (m, 1H), 2.07-2.00 (m, 1H) |

### INTERMEDIATE AA: trans-4-[5-(4-[[4-(prop-2-yn-1-yl)piperazin-1-yl]methyl]phenyl)-2-[(3,3,3-trifluoropropyl)amino]-7H-pyrrolo[2,3-d]pyrimidin-7-yl]cyclohexan-1-ol

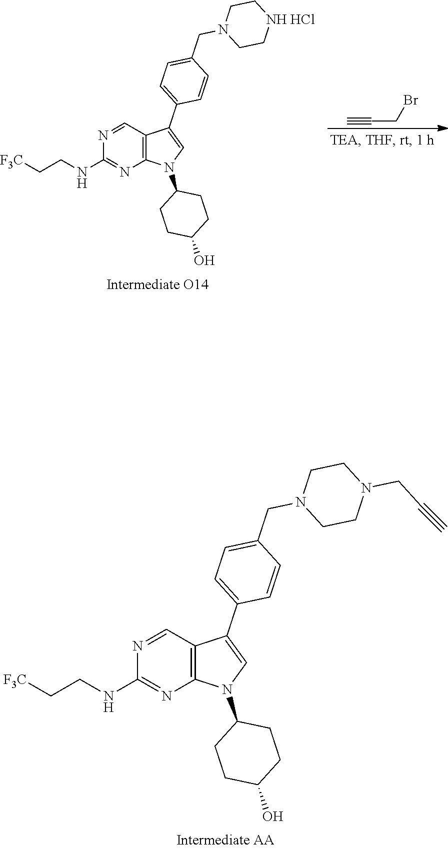

To a stirred solution of trans-4-(5-[4-[(piperazin-1-yl)methyl]phenyl]-2-[(3,3,3-trifluoropropyl)amino]-7H-pyrrolo[2,3-d]pyrimidin-7-yl)cyclohexan-1-ol (400 mg, 0.80 mmol) and 3-bromoprop-1-yne (947 mg, 7.96 mmol) in THF (40.0 mL) was added TEA (805 mg, 7.96 mmol) at room temperature under nitrogen atmosphere. After stirring for additional 1 h, the resulting solution was concentrated under reduced pressure. The residue was purified by reversed phase flash chromatography with the following conditions: Column: WelFlash™ C18-I, 20-40 μm, 120 g; Eluent A: water (plus 10 mmol/L NH₄HCO₃); Eluent B: ACN; Gradient: 40%-60% B in 25 min; Flow rate: 80 mL/min; Detector: UV 220/254 nm; desired fractions were collected at 57% B and concentrated under reduced pressure to afford trans-4-[5-(4-[[4-(prop-2-yn-1-yl)piperazin-1-yl]methyl]phenyl)-2-[(3,3,3-trifluoropropyl)amino]-7H-pyrrolo[2,3-d]pyrimidin-7-yl]cyclohexan-1-ol (160 mg, 37%) as a colorless solid. ¹H NMR (400 MHz, DMSO-d₆) δ 8.88 (s, 1H), 7.64 (m, 3H), 7.32 (d, J=8.1 Hz, 2H), 7.06 (t, J=5.9 Hz, 1H), 4.71 (d, J=4.4 Hz, 1H), 4.50-4.40 (m, 1H), 3.60-3.50 (m, 3H), 3.46 (s, 2H), 3.25 (d, J=2.5 Hz, 2H), 3.15 (t, J=2.4 Hz, 1H), 2.65-2.50 (m, 2H), 1.98 (d, J=11.7 Hz, 3H), 1.94-1.81 (m, 3H), 1.40-1.30 (m, 2H). LC/MS (ESI, m/z): [(M+1)]⁺=541.30.

### SYNTHESIS OF FINAL TARGETS

### EXAMPLE 1: 4-((4-(6-(Butylamino)-1-(trans-4-hydroxycyclohexyl)-1H-pyrazolo[3,4-d]pyrimidin-3-yl)phenyl)sulfonyl)-N-(3-(3-(3-(1-(2,6-dioxopiperidin-3-yl)-3-methyl-2-oxo-2,3-dihydro-1H-benzo[d]imidazol-4-yl)propoxy)propoxy)propyl)piperazine-1-carboxamide (I-29)

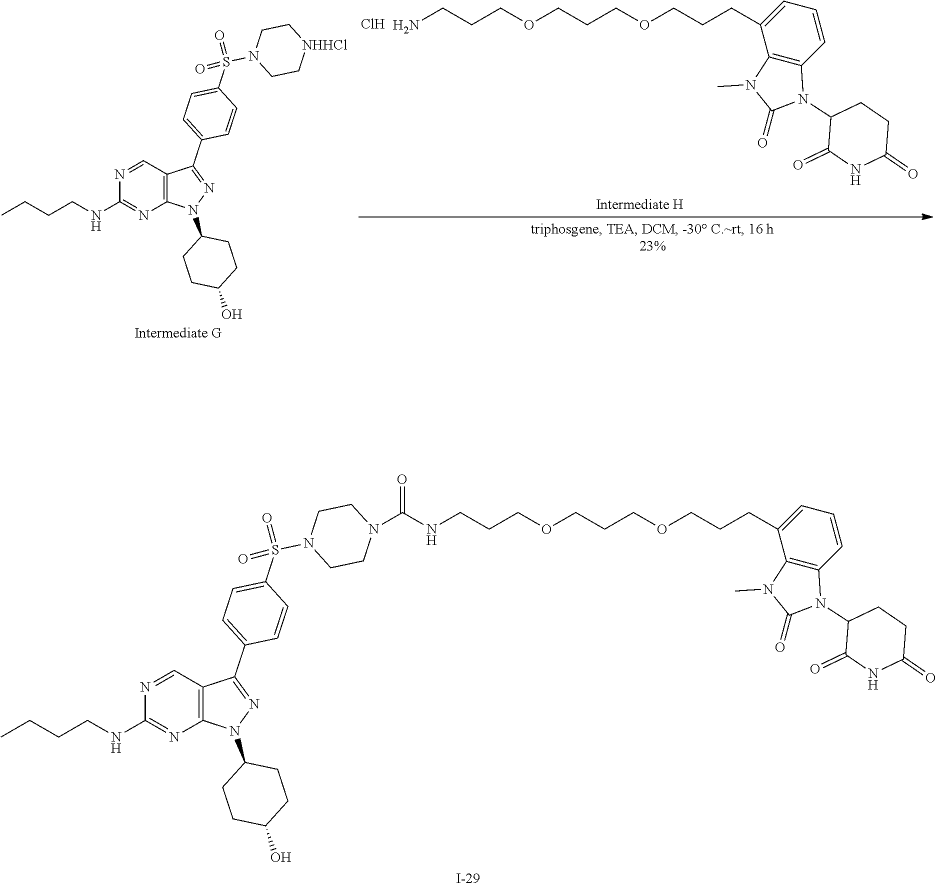

To a stirred solution of trans-4-[6-(butylamino)-3-[4-(piperazine-1-sulfonyl)phenyl]-1H-pyrazolo[3,4-d]pyrimidin-1-yl]cyclohexan-1-ol hydrochloride (100 mg, 0.18 mmol) in DCM (10 mL) were added TEA (184 mg, 1.82 mmol), 3-(4-[3-[3-(3-aminopropoxy)propoxy]propyl]-3-methyl-2-oxo-2,3-dihydro-1H-1,3-benzodiazol-1-yl)piperidine-2,6-dione hydrochloride (85 mg, 0.18 mmol) and a solution of triphosgene (16 mg, 0.055 mmol) in DCM (3 mL) dropwise at −30° C. under nitrogen atmosphere. The resulting solution was stirred at room temperature for 16 h. The resulting solution was concentrated under reduced pressure. The residue was purified by reverse phase flash chromatography with the following conditions: Column: WelFlash™ C18-I, 20-40 μm, 80 g; Eluent A: Water (plus 10 mmol/L HCOOH); Eluent B: ACN; Gradient: 30%-55% B in 25 min; Flow rate: 40 mL/min; Detector: 220/254 nm; desired fractions were collected at 46% B and concentrated under reduced pressure to afford 4-[4-[6-(butylamino)-1-[trans-4-hydroxycyclohexyl]-1H-pyrazolo[3,4-d]pyrimidin-3-yl]benzenesulfonyl]-N-[3-(3-[3-[1-(2,6-dioxopiperidin-3-yl)-3-methyl-2-oxo-2,3-dihydro-1H-1,3-benzodiazol-4-yl]propoxy]propoxy)propyl]piperazine-1-carboxamide (40 mg, 23%) as a white solid. ¹H NMR (400 MHz, DMSO-d₆) δ 11.09 (s, 1H), 9.17 (s, 1H), 8.22 (d, J=8.4 Hz, 2H), 7.82 (d, J=8.4 Hz, 2H), 7.67 (s, 1H), 7.05-6.90 (m, 2H), 6.84 (dd, J=6.3, 2.6 Hz, 1H), 6.52 (t, J=5.3 Hz, 1H), 5.37 (dd, J=12.7, 5.4 Hz, 1H), 4.71 (s, 1H), 4.53 (s, 1H), 3.54 (s, 4H), 3.45-3.30 (m, 12H), 3.27 (t, J=6.3 Hz, 2H), 2.99 (q, J=6.5 Hz, 2H), 2.94-2.83 (m, 7H), 2.74-2.59 (m, 2H), 2.10-1.86 (m, 7H), 1.79 (t, J=7.7 Hz, 2H), 1.66 (q, J=6.3 Hz, 2H), 1.62-1.50 (m, 4H), 1.48-1.30 (m, 4H), 0.93 (s, 3H). LC/MS (ESI, m/z): [(M+1)]⁺=972.65

The following compounds, 1-19, 1-20, 1-23, and 1-24, were prepared according to the above procedure.

### EXAMPLE 2: 4-((4-(6-(Butylamino)-1-(trans-4-hydroxycyclohexyl)-1H-pyrazolo[3,4-d]pyrimidin-3-yl)phenyl)sulfonyl)-N-(3-(3-(3-(1-(2,6-dioxopiperidin-3-yl)-3-methyl-2-oxo-2,3-dihydro-1H-benzo[d]imidazol-5-yl)propoxy)propoxy)propyl)piperazine-1-carboxamide (I-24)

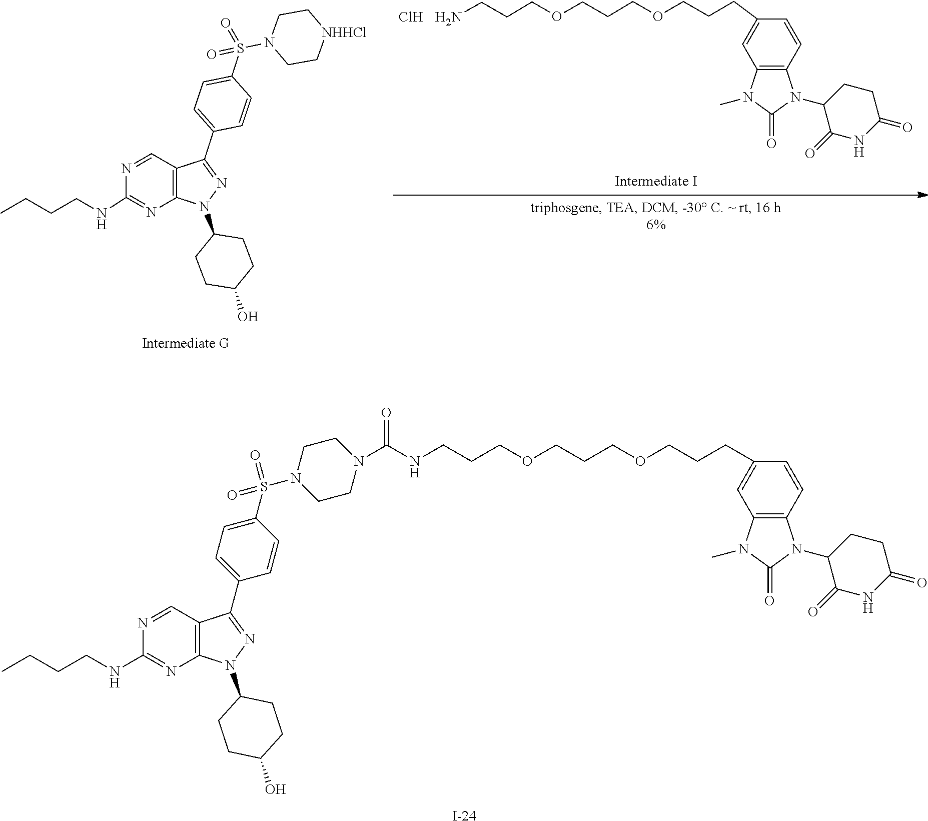

(10 mg, 6%) as a white solid. ¹H NMR (400 MHz, DMSO-d₆) δ 11.08 (br s, 1H), 9.18 (s, 1H), 8.23 (d, J=8.4 Hz, 2H), 7.82 (d, J=8.4 Hz, 2H), 7.66 (br s, 1H), 7.02 (s, 1H), 7.00 (d, J=8.0 Hz, 1H), 6.84 (d, J=8.0 Hz, 1H), 6.52 (t, J=5.4 Hz, 1H), 5.34 (dd, J=12.7, 5.4 Hz, 1H), 4.71 (d, J=4.3 Hz, 1H), 4.60-4.48 (m, 1H), 3.63-3.48 (m, 2H), 3.43-3.24 (m, 10H), 3.03-2.95 (m, 2H), 2.95-2.82 (m, 5H), 2.75-2.50 (m, 4H), 2.11-1.90 (m, 7H), 1.82-1.73 (m, 2H), 1.70-1.60 (m, 2H), 1.60-1.45 (m, 4H), 1.42-1.30 (m, 5H), 1.28-1.16 (m, 2H), 1.08-0.62 (m, 5H). LC/MS (ESI, m/z): [(M+1)]⁺=972.60.

### EXAMPLE 3: 4-((4-(6-(Butylamino)-1-(trans-4-hydroxycyclohexyl)-1H-pyrazolo[3,4-d]pyrimidin-3-yl)phenyl)sulfonyl)-N-(3-(3-((2-(2,6-dioxopiperidin-3-yl)-1,3-dioxoisoindolin-4-yl)amino)propoxy)propyl)piperazine-1-carboxamide (I-20)

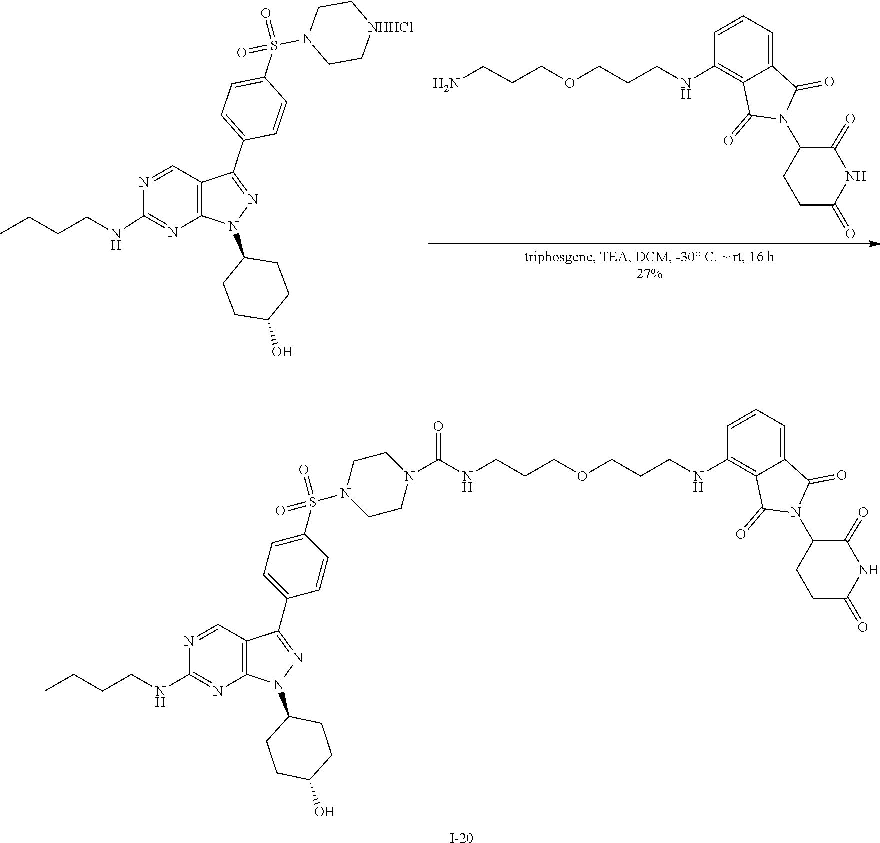

(15 mg, 27%) as a light yellow solid. ¹H NMR (400 MHz, DMSO-d₆) δ 9.17 (br s, 1H), 8.22 (d, J=8.4 Hz, 2H), 7.82 (d, J=8.4 Hz, 2H), 7.72-7.50 (m, 2H), 7.06-6.96 (m, 2H), 6.61 (t, J=6.1 Hz, 1H), 6.52 (s, 1H), 5.04 (dd,J=12.8, 5.4 Hz, 1H), 4.75-4.63 (m, 1H), 4.60-4.43 (m, 1H), 3.60-3.50 (m, 2H), 3.50-3.40 (m, 4H), 3.06-2.78 (m, 8H), 2.63-2.52 (m, 2H), 2.39-1.83 (m, 9H), 1.81-1.12 (m, 13H), 1.00-0.80 (m, 4H). LC/MS (ESI, m/z): [(M+1)]⁺=928.45.

### EXAMPLE 4: 4-((4-(6-(Butylamino)-1-(trans-4-hydroxycyclohexyl)-1H-pyrazolo[3,4-d]pyrimidin-3-yl)phenyl)sulfonyl)-N-(2-(3-(2-((2-(2,6-dioxopiperidin-3-yl)-1,3-dioxoisoindolin-4-yl)amino)ethoxy)propoxy)ethyl)piperazine-1-carboxamide (I-23)

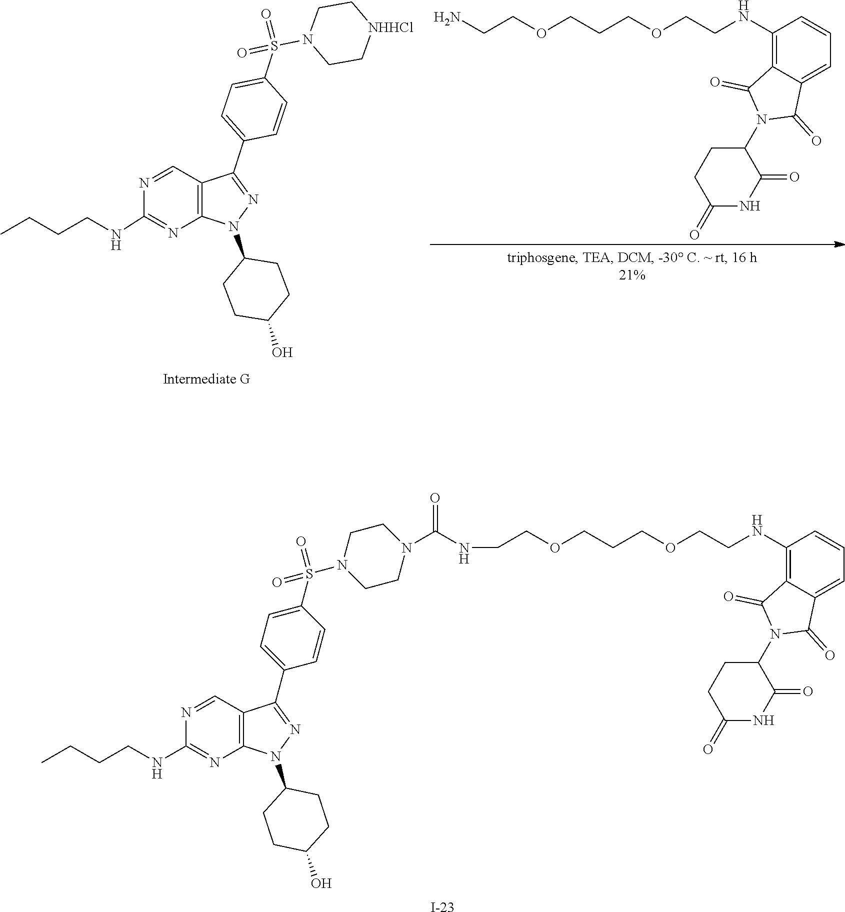

(13 mg, 21%) as a light yellow solid. ¹H NMR (400 MHz, DMSO-d₆) δ 11.09 (s, 1H), 9.17 (s, 1H), 8.22 (d, J=8.4 Hz, 2H), 7.82 (d, J=8.4 Hz, 2H), 7.66 (br s, 1H), 7.60-7.52 (m, 1H), 7.10 (d, J=8.4 Hz, 1H), 7.03 (d, J=6.8 Hz, 1H), 6.61-6.52 (m, 2H), 5.05 (dd, J=12.9, 5.4 Hz, 1H), 4.70 (d, J=4.3 Hz, 1H), 4.60-4.50 (m, 1H), 3.60-3.46 (m, 3H), 3.43-3.34 (m, 10H), 3.25 (t, J=6.0 Hz, 2H), 3.07 (q, J=5.8 Hz, 2H), 2.93-2.80 (m, 5H), 2.70-2.50 (m, 2H), 2.25-1.81 (m, 8H), 1.65 (q, J=6.3 Hz, 2H), 1.62-1.52 (m, 2H), 1.45-1.30 (m, 4H), 1.00-0.89 (m, 3H). LC/MS (ESI, m/z): [(M+1)]⁺=958.55.

### EXAMPLE 5: 4-((4-(6-(Butylamino)-1-(trans-4-hydroxycyclohexyl)-1H-pyrazolo[3,4-d]pyrimidin-3-yl)phenyl)sulfonyl)-N-(3-(3-(3-((2-(2,6-dioxopiperidin-3-yl)-1,3-dioxoisoindolin-4-yl)amino)propoxy)propoxy)propyl)piperazine-1-carboxamide (I-19)

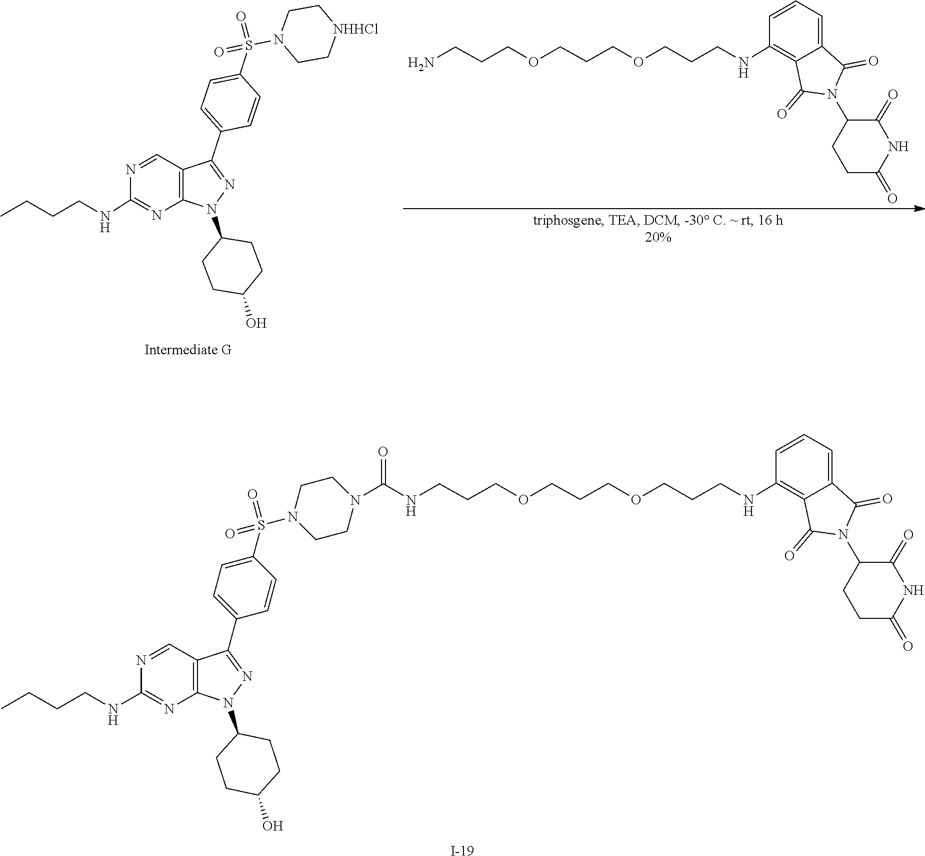

(10 mg, 20%) as a light yellow solid. ¹H NMR (400 MHz, DMSO-d₆) δ 11.09 (s, 1H), 9.18 (s, 1H), 8.49 (s, 1H), 8.23 (d, J=8.4 Hz, 2H), 7.82 (d, J=8.4 Hz, 2H), 7.71-7.52 (m, 2H), 7.08-7.04 (m, 2H), 6.63 (t, J=5.8 Hz, 1H), 6.50 (s, 1H), 5.04 (dd, J=12.9, 5.4 Hz, 1H), 4.71 (s, 1H), 4.60-4.49 (m, 1H), 3.62-3.50 (m, 1H), 3.50-3.40 (m, 5H), 3.30-3.20 (m, 4H), 3.03-2.80 (m, 9H), 2.62-2.50 (m, 2H), 2.12-1.90 (m, 8H), 1.81-1.50 (m, 10H), 1.48-1.32 (m, 4H), 1.00-0.89 (m, 4H). LC/MS (ESI, m/z): [(M+1)]⁺=986.55

### EXAMPLE 6: N-(11-(4-((4-(6-(Butylamino)-1-(trans-4-hydroxycyclohexyl)-1H-pyrazolo[3,4-d]pyrimidin-3-yl)phenyl)sulfonyl)piperazin-1-yl)-11-oxoundecyl)-2-((2-(2,6-dioxopiperidin-3-yl)-1,3-dioxoisoindolin-4-yl)oxy)acetamide (I-28)

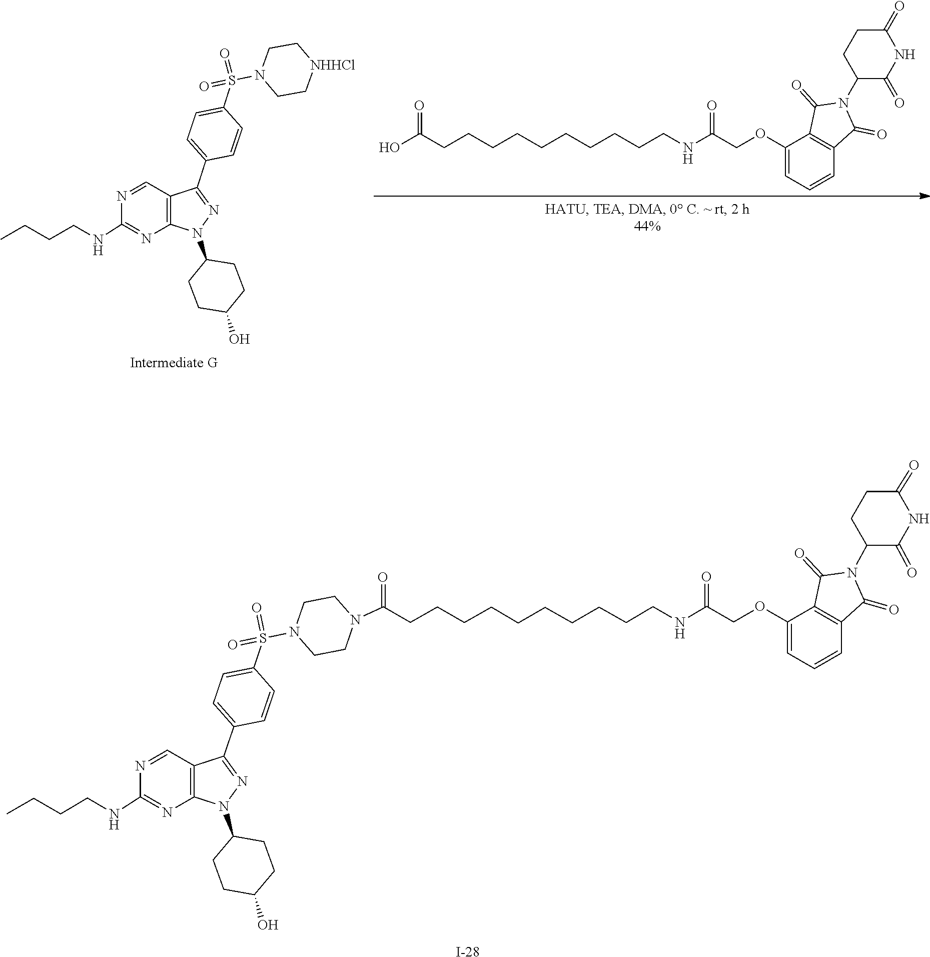

To a stirred solution of trans-4-[6-(butylamino)-3-[4-(piperazine-1-sulfonyl)phenyl]-1H-pyrazolo[3,4-d]pyrimidin-1-yl]cyclohexan-1-ol hydrochloride (50 mg, 0.091 mmol) in DMA (3 mL) were added 11-(2-[[2-(2,6-dioxopiperidin-3-yl)-1,3-dioxo-2,3-dihydro-1H-isoindol-4-yl]oxy]acetamido)undecanoic acid (47 mg, 0.091 mmol), TEA (28 mg, 0.27 mmol) and HATU (42 mg, 0.11 mmol) at 0° C. under nitrogen atmosphere. The resulting solution was stirred for 2 h at room temperature. The mixture was purified by prep-HPLC with the following conditions: column: X Select CSH Prep C¹⁸ OBD Column, 5 um, 19×150 mm; Mobile Phase A: water (plus 0.1% FA); Mobile Phase B: ACN; Flow rate: 20 mL/min; Gradient: 50% B to 65% B in 7 min; Detector: 254/220 nm; Rt: 6.27 min. The fractions containing desired product were combined and concentrated under reduced pressure to afford N-(11-(4-((4-(6-(butylamino)-1-(trans-4-hydroxycyclohexyl)-1H-pyrazolo[3,4-d]pyrimidin-3-yl)phenyl)sulfonyl)piperazin-1-yl)-11-oxoundecyl)-2-((2-(2,6-dioxopiperidin-3-yl)-1,3-dioxoisoindolin-4-yl)oxy)acetamide (40 mg, 44%) as a white solid. ¹H NMR (400 MHz, DMSO-d₆) δ 11.12 (s, 1H), 9.19 (s, 1H), 8.23 (d, J=8.4 Hz, 2H), 7.91 (t, J=5.7 Hz, 1H), 7.85-7.76 (m, 3H), 7.67 (br s, 1H), 7.49 (d, J=7.2 Hz, 1H), 7.38 (d, J=8.5 Hz, 1H), 5.12 (dd, J=12.9, 5.3 Hz, 1H), 4.76 (s, 2H), 4.56-4.46 (m, 1H), 3.60-3.50 (m, 6H), 3.40-3.35 (m, 2H), 3.11 (q, J=6.6 Hz, 2H), 2.97-2.86 (m, 5H), 2.65-2.55 (m, 1H), 2.21 (t, J=7.5 Hz, 2H), 2.10-1.89 (m, 7H), 1.53-1.55 (m, 2H), 1.40-1.35 (m, 8H), 1.20-1.10 (m, 12H), 0.94 (t, J=7.1 Hz, 3H). LC/MS (ESI, m/z): [(M+1)]⁺=1011.65.

### EXAMPLE 7: (2S,4R)-1-((S)-2-(12-(4-((4-(6-(Butylamino)-1-(trans-4-hydroxycyclohexyl)-1H-pyrazolo[3,4-d]pyrimidin-3-yl)phenyl)sulfonyl)piperazin-1-yl)-12-oxododecanamido)-3,3-dimethylbutanoyl)-4-hydroxy-N-(4-(4-methylthiazol-5-yl)benzyl)pyrrolidine-2-carboxamide (I-27)

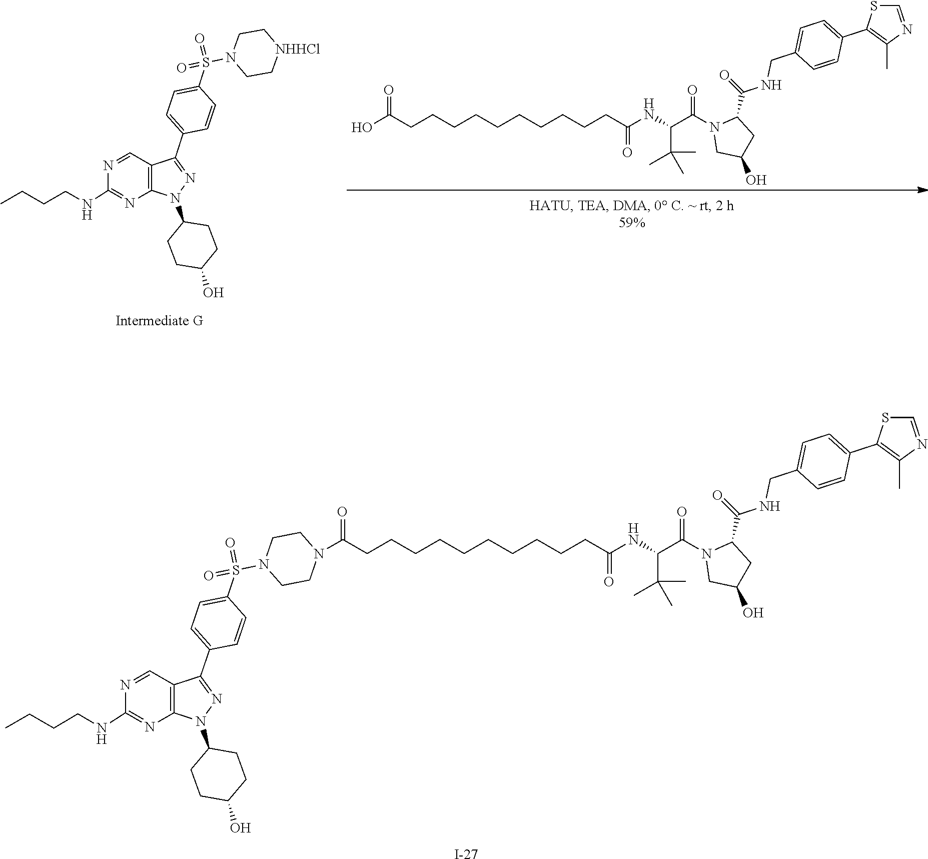

To a stirred solution of trans-4-[6-(butylamino)-3-[4-(piperazine-1-sulfonyl)phenyl]-1H-pyrazolo[3,4-d]pyrimidin-1-yl]cyclohexan-1-ol hydrochloride (45 mg, 0.082 mmol) in DMA (3 mL) were added 11-[[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamoyl]undecanoic acid (53 mg, 0.082 mmol), TEA (25 mg, 0.25 mmol) and HATU (38 mg, 0.098 mmol) at 0° C. under nitrogen atmosphere. The resulting solution was stirred for 2 h at room temperature. The mixture was purified by prep-HPLC with the following conditions: column: X Select CSH Prep C¹⁸ OBD column, 5 um, 19×150 mm; Mobile Phase A: Water (plus 0.1% FA), Mobile Phase B: ACN; Flow rate: 20 mL/min; Gradient: 50% B to 75% B in 7 min; Detector: 254/220 nm; Rt: 5.78 min. The fractions containing desired product were combined and concentrated under reduced pressure to afford (2S,4R)-1-((S)-2-(12-(4-((4-(6-(butylamino)-1-(trans-4-hydroxycyclohexyl)-1H-pyrazolo[3,4-d]pyrimidin-3-yl)phenyl)sulfonyl)piperazin-1-yl)-12-oxododecanamido)-3,3-dimethylbutanoyl)-4-hydroxy-N-(4-(4-methylthiazol-5-yl)benzyl)pyrrolidine-2-carboxamide (55 mg, 59%) as a white solid. ¹H NMR (400 MHz, DMSO-d₆) δ 9.21 (br s, 1H), 9.01 (s, 1H), 8.56 (t, J=6.1 Hz, 1H), 8.23 (d, J=8.8 Hz, 2H), 7.84-7.81 (m, 3H), 7.76-7.61 (m, 1H), 7.42 (d, J=8.4 Hz, 2H), 7.38 (d, J=8.4 Hz, 2H), 4.57-4.50 (m, 2H), 4.49-4.38 (m, 2H), 4.35 (s, 1H), 4.25-4.18 (m, 1H), 3.71-3.59 (m, 2H), 3.58-3.47 (m, 5H), 3.42-3.35 (m, 2H), 2.97-2.84 (m, 4H), 2.45 (s, 3H), 2.27-2.19 (m, 3H), 2.14-1.87 (m, 9H), 1.63-1.52 (m, 2H), 1.50-1.30 (m, 8H), 1.23-1.10 (m, 12H), 0.98-0.88 (m, 12H). LC/MS (ESI, m/z): [(M+1)]⁺=1138.70

### EXAMPLE 8: 2-(2,6-Dioxopiperidin-3-yl)-4-(15-[4-[(4-[2-[(2-methoxyethyl)amino]-7-[trans-4-hydroxycyclohexyl]-7H-pyrrolo[2,3-d]pyrimidin-5-yl]phenyl)methyl]piperazin-1-yl]-4,7,10,13-tetraoxa-1-azapentadecan-1-yl)-2,3-dihydro-1H-isoindole-1,3-dione diformate (I-15)

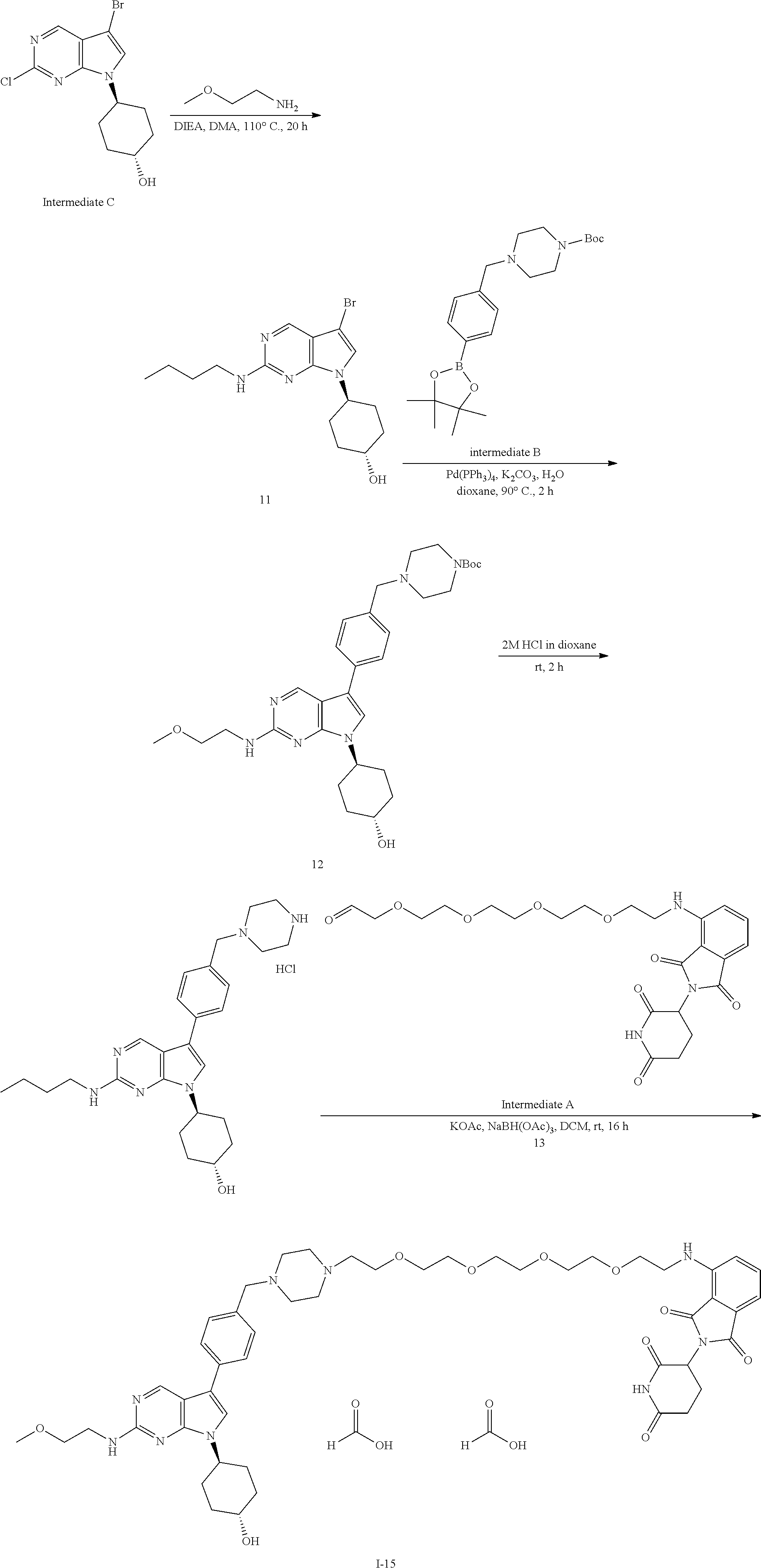

### Step 1: trans-4-[5-Bromo-2-[(2-methoxyethyl)amino]-7H-pyrrolo[2,3-d]pyrimidin-7-yl]cyclohexan-1-ol

To a stirred solution of trans-4-[5-bromo-2-chloro-7H-pyrrolo[2,3-d]pyrimidin-7-yl]cyclohexan-1-ol (300 mg, 0.91 mmol) and DIEA (293.19 mg, 2.27 mmol) in NMP (5 mL) was added 2-methoxyethan-1-amine (204 mg, 2.72 mmol) at room temperature. The reaction was stirred at 110° C. for 20 h. The resulting mixture was cooled down to room temperature and was purified by reverse phase flash chromatography with the following conditions: Column: WelFlash™ C18-I, 20-40 μm, 120 g; Eluent A: Water (plus 10 mmol/L NH₄HCO₃); Eluent B: ACN; Gradient: 40%-60% B in 20 min; Flow rate: 45 mL/min; Detector: 220/254 nm; desired fractions were collected at 52% B and concentrated under reduced pressure to afford trans-4-[5-bromo-2-[(2-methoxyethyl)amino]-7H-pyrrolo[2,3-d]pyrimidin-7-yl]cyclohexan-1-ol as an orange solid (270 mg, 81%). ¹H NMR (400 MHz, DMSO-d₆) δ 8.37 (s, 1H), 7.40 (s, 1H), 6.93 (s, 1H), 4.68 (d, J=4.4 Hz, 1H), 4.39 (dq, J=11.4, 6.8, 5.7 Hz, 1H), 3.55-3.45 (m, 5H), 3.28 (s, 3H), 2.00-1.60 (m, 6H), 1.40-1.25 (m, 2H). LC/MS (ESI, m/z): [(M+1)]⁺=369.08, 371.08

### Step 2: tert-Butyl 4-[(4-[2-[(2-methoxyethyl)amino]-7-[trans-4-hydroxycyclohexy]-7H-pyrrolo[2,3-d]pyrimidin-5-yl]phenyl)methyl]piperazine-1-carboxylate

To a stirred solution of trans-4-[5-bromo-2-[(2-methoxyethyl)amino]-7H-pyrrolo[2,3-d]pyrimidin-7-yl]cyclohexan-1-ol (270 mg, 0.73 mmol) in dioxane (4 mL) were added H₂O (1 mL), tert-butyl 4-[[4-(4,4,5,5-tetramethyl-1,3,2-dioxaborolan-2-yl)phenyl]methyl]piperazine-1-carboxylate (294 mg, 0.73 mmol), K₂CO₃ (151 mg, 1.09 mmol) and Pd(PPh₃)₄ (42 mg, 0.037 mmol.) at room temperature under nitrogen atmosphere. The resulting mixture was stirred at 90° C. for 2 h. The resulting mixture was cooled and concentrated under reduced pressure. The residue was taken up with water (10 mL) and ethyl acetate (30 mL). The organic phase was separated and dried over anhydrous Na₂SO₄. After filtration, the filtrate was concentrated under reduced pressure. The residue was purified by silica gel column chromatography, eluted with 1%-7% methanol in dichloromethane. The fractions with desired product were combined and concentrated under reduced pressure to afford tert-butyl 4-[(4-[2-[(2-methoxyethyl)amino]-7-[trans-4-hydroxycyclohexyl]-7H-pyrrolo[2,3-d]pyrimidin-5-yl]phenyl)methyl]piperazine-1-carboxylate (260 mg, 63%) as a yellow solid. ¹H NMR (400 MHz, DMSO-d₆) δ 8.84 (s, 1H), 7.71-7.55 (m, 3H), 7.32 (d, J=8.0 Hz, 2H), 6.77 (s, 1H), 4.70 (d, J=4.5 Hz, 1H), 4.58-4.33 (m, 1H), 3.93 (s, 6H), 3.60-3.45 (m, 9H), 3.29 (s, 3H), 2.38-2.29 (m, 4H), 2.06-1.81 (m, 2H), 1.41-1.37 (m, 11H). LC/MS (ESI, m/z): [(M+1)]⁺=565.34

### Step 3: trans-4-[2-[(2-Methoxyethyl)amino]-5-[4-[(piperazin-1-yl)methyl]phenyl]-7H-pyrrolo[2,3-d]pyrimidin-7-yl]cyclohexan-1-ol hydrochloride

To a stirred solution of tert-butyl 4-[(4-[2-[(2-methoxyethyl)amino]-7-[trans-4-hydroxycyclohexyl]-7H-pyrrolo[2,3-d]pyrimidin-5-yl]phenyl)methyl]piperazine-1-carboxylate (210 mg, 0.37 mmol) in dioxane (5 mL) was added a solution of hydrochloride in 1,4-dioxane (4 M, 5 mL) at room temperature. The resulting solution was stirred at room temperature for 2 h. The resulting mixture was concentrated under reduced pressure to afford trans-4-[2-[(2-methoxyethyl)amino]-5-[4-[(piperazin-1-yl)methyl]phenyl]-7H-pyrrolo[2,3-d]pyrimidin-7-yl]cyclohexan-1-ol hydrochloride as a yellow solid (210 mg, 100%). ¹H NMR (400 MHz, DMSO-d₆) δ 9.85 (br s, 2H), 9.14 (s, 1H), 8.21 (s, 1H), 8.13 (s, 1H), 7.83 (d, J=8.0 Hz, 2H), 7.76 (d, J=8.0 Hz, 2H), 4.55-4.40 (m, 2H), 4.30-4.10 (m, 2H), 3.70-3.63 (m, 2H), 3.63-3.54 (m, 3H), 3.54-3.43 (m, 10H), 2.10-1.80 (m, 6H), 1.45-1.30 (m, 2H). LC/MS (ESI, m/z): [(M+1)]⁺=465.34

### Step 4: 2-(2,6-Dioxopiperidin-3-yl)-4-(15-[4-[(4-[2-[(2-methoxyethyl)amino]-7-[trans-4-hydroxycyclohexyl]-7H-pyrrolo[2,3-d]pyrimidin-5-yl]phenyl)methyl]piperazin-1-yl]-4,7,10,13-tetraoxa-1-azapentadecan-1-yl)-2,3-dihydro-1H-isoindole-1,3-dione diformate

To a stirred solution of trans-4-[2-[(2-methoxyethyl)amino]-5-[4-[(piperazin-1-yl)methyl]phenyl]-7H-pyrrolo[2,3-d]pyrimidin-7-yl]cyclohexan-1-ol hydrochloride (50 mg, 0.11 mmol) in DCM (5 mL) were added 14-[[2-(2,6-dioxopiperidin-3-yl)-1,3-dioxo-2,3-dihydro-1H-isoindol-4-yl]amino]-3,6,9,12-tetraoxatetradecanal (53 mg, 0.11 mmol), KOAc (42 mg, 0.43 mmol) and NaBH(OAc)₃ (68 mg, 0.32 mmol) at room temperature. The resulting mixture was stirred for an additional 16 h at room temperature. The resulting mixture was concentrated under reduced pressure. The residue was purified by reverse phase Flash column chromatography with the following conditions: Column: WelFlash™ C18-I, 20-40 μm, 80 g; Eluent A: Water (plus 10 mmol/L HCOOH); Eluent B: ACN; Gradient: 45%-65% B in 20 min; Flow rate: 45 mL/min; Detector: 220/254 nm; desired fractions were collected at 60% B and concentrated under reduced pressure to afford 2-(2,6-dioxopiperidin-3-yl)-4-(15-[4-[(4-[2-[(2-methoxyethyl)amino]-7-[trans-4-hydroxycyclohexyl]-7H-pyrrolo [2,3-d]pyrimidin-5-yl]phenyl)methyl]piperazin-1-yl]-4,7,10,13-tetraoxa-1-azapentadecan-1-yl)-2,3-dihydro-1H-isoindole-1,3-dione diformate (25 mg, 24%) as a yellow solid. ¹H NMR (400 MHz, CD₃OD) δ 8.73 (s, 1H), 8.34 (s, 2H), 7.62 (d, J=8.0 Hz, 2H), 7.51 (dd, J=8.5, 7.1 Hz, 1H), 7.46 (s, 1H), 7.40 (d, J=8.0 Hz, 2H), 7.03 (dd, J=7.8, 3.6 Hz, 2H), 5.04 (dd, J=12.7, 5.5 Hz, 1H), 4.55 (tt, J=10.4, 5.4 Hz, 1H), 3.81-3.69 (m, 5H), 3.68-3.59 (m, 18H), 3.50-3.40 (m, 5H), 3.27-3.16 (m, 6H), 2.92-2.60 (m, 7H), 2.20-1.90 (m, 7H), 1.54 (qd, J=11.8, 5.2 Hz, 2H). LC/MS (ESI, m/z): [(M+1)]⁺=940.49

### EXAMPLE 9: 4-(15-[4-[(4-[2-[(2-Cyclopropylethyl)amino]-7-[trans-4-hydroxycyclohexyl]-7H-pyrrolo[2,3-d]pyrimidin-5-yl]phenyl)methyl]piperazin-1-yl]-4,7,10,13-tetraoxa-1-azapentadecan-1-yl)-2-(2,6-dioxopiperidin-3-yl)-2,3-dihydro-1H-isoindole-1,3-dione formate (I-14)

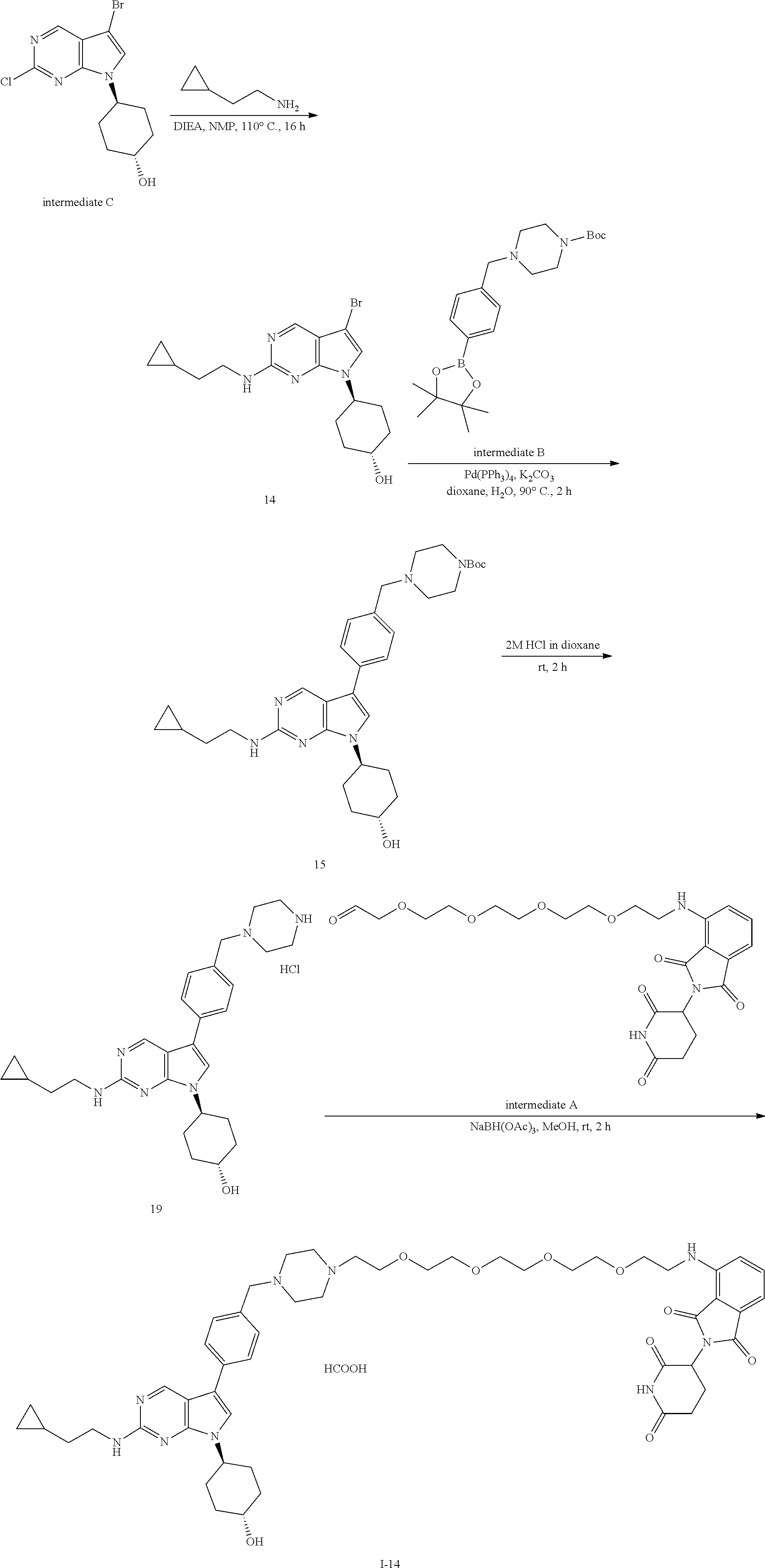

### Step 1: trans-4-[5-Bromo-2-[(2-methoxyethyl)amino]-7H-pyrrolo[2,3-d]pyrimidin-7-yl]cyclohexan-1-ol

To a stirred solution of trans-4-[5-bromo-2-chloro-7H-pyrrolo[2,3-d]pyrimidin-7-yl]cyclohexan-1-ol (300 mg, 0.91 mmol) in NMP (5 mL) were added DIEA (293 mg, 2.27 mmol) and 2-methoxyethan-1-amine (204 mg, 2.72 mmol) at room temperature. The resulting mixture was stirred at 110° C. for 16 h. After cooling down to room temperature, the resulting solution was applied onto a C18 column and purified by reverse phase Flash column chromatography with the following conditions: Column: WelFlash™ C18-I, 20-40 μm, 120 g; Eluent A: Water (plus 10 mmol/L NH4HCO₃); Eluent B: ACN; Gradient: 40%-60% B in 20 min; Flow rate: 45 mL/min; Detector: 220/254 nm; desired fractions were collected at 50% B and concentrated under reduced pressure to afford trans-4-[5-bromo-2-[(2-methoxyethyl)amino]-7H-pyrrolo[2,3-d]pyrimidin-7-yl]cyclohexan-1-ol as an orange solid (270 mg, 81%). ¹H NMR (400 MHz, DMSO-d₆) δ 8.35 (s, 1H), 7.37 (s, 1H), 7.00 (t, J=5.6 Hz, 1H), 4.69 (s, 1H), 4.38 (dq, J=11.3, 6.5, 5.7 Hz, 1H), 3.50 (d, J=11.3 Hz, 1H), 3.41-3.33 (m, 2H), 2.01-1.78 (m, 6H), 1.46 (q, J=7.2 Hz, 2H), 1.41-1.28 (m, 2H), 0.74 (tt, J=7.8, 4.8 Hz, 1H), 0.45-0.36 (m, 2H), 0.07 (dd, J=5.4, 3.8 Hz, 2H). LC/MS (ESI, m/z): [(M+1)]⁺=379.00.

### Step 2: tert-Butyl 4-[(4-[2-[(2-cyclopropylethyl)amino]-7-[trans-4-hydroxycyclohexyl]-7H-pyrrolo[2,3-d]pyrimidin-5-yl]phenyl)methyl]piperazine-1-carboxylate

To a stirred solution of trans-4-[5-bromo-2-[(2-cyclopropylethyl)amino]-7H-pyrrolo[2,3-d]pyrimidin-7-yl]cyclohexan-1-ol (370 mg, 0.98 mmol) in dioxane (4 mL) were added H₂O (1.5 mL), tert-butyl 4-[[4-(4,4,5,5-tetramethyl-1,3,2-dioxaborolan-2-yl)phenyl]methyl]piperazine-1-carboxylate (785 mg, 1.95 mmol), K₂CO₃ (202 mg, 1.46 mmol) and Pd(PPh₃)₄ (56 mg, 0.049 mmol) at room temperature under nitrogen atmosphere. The resulting mixture was stirred at 90° C. for 2 h. Upon completion, the resulting mixture was cooled down to room temperature and concentrated under reduced pressure. The residue was taken up with water (10 mL) and ethyl acetate (30 mL). The organic phase was separated and dried over anhydrous Na₂SO₄. After filtration, the filtrate was concentrated under reduced pressure. The residue was purified by silica gel column chromatography, eluted with 1%-7% methanol in dichloromethane. The fractions containing the desired product were collected and concentrated under reduced pressure to afford tert-butyl 4-[(4-[2-[(2-cyclopropylethyl)amino]-7-[trans-4-hydroxycyclohexyl]-7H-pyrrolo[2,3-d]pyrimidin-5-yl]phenyl)methyl]piperazine-1-carboxylate as a yellow solid (190 mg, 34%). ¹H NMR (400 MHz, DMSO-d₆) δ 8.82 (s, 1H), 7.63 (d, J=8.0 Hz, 2H), 7.58 (s, 1H), 7.32 (d, J=7.9 Hz, 2H), 6.83 (t, J=5.9 Hz, 1H), 4.70 (d, J=4.4 Hz, 1H), 4.52-4.35 (m, 1H), 3.62-3.51 (m, 1H), 3.48 (s, 2H), 3.45-3.35 (m, 2H), 3.34 (t, J=5.0 Hz, 4H), 2.33 (t, J=5.0 Hz, 4H), 2.05-1.84 (m, 6H), 1.48 (q, J=7.2 Hz, 2H), 1.44-1.30 (m, 2H), 1.40 (s, 9H), 0.77 (dq, J=12.8, 7.6, 6.4 Hz, 1H), 0.51-0.38 (m, 2H), 0.15-0.04 (m, 2H). LC/MS (ESI, m/z): [(M+1)]⁺=675.36

### Step 3: trans-4-[2-[(2-cyclopropylethyl)amino]-5-[4-[(piperazin-1-yl)methyl]phenyl]-7H-pyrrolo[2,3-d]pyrimidin-7-y]cyclohexan-1-ol hydrochloride

To a stirred solution of tert-butyl 4-[(4-[2-[(2-cyclopropylethyl)amino]-7-[trans-4-hydroxycyclohexyl]-7H-pyrrolo[2,3-d]pyrimidin-5-yl]phenyl)methyl]piperazine-1-carboxylate (190 mg, 0.33 mmol) in dioxane (5 mL) was added a solution of hydrochloride in 1,4-dioxane (4 M, 5 mL) at room temperature. The resulting solution was stirred at room temperature for 2 h. The resulting solution was concentrated under reduced pressure to afford trans-4-[2-[(2-cyclopropylethyl)amino]-5-[4-[(piperazin-1-yl)methyl]phenyl]-7H-pyrrolo[2,3-d]pyrimidin-7-yl]cyclohexan-1-ol hydrochloride as a yellow solid (150 mg, 89%). ¹H NMR (400 MHz, DMSO-d₆) δ 12.27 (br s, 1H), 9.74 (br s, 2H), 9.12 (s, 1H), 8.20 (s, 1H), 7.83 (d, J=7.9 Hz, 2H), 7.75 (d, J=7.9 Hz, 2H), 4.55-4.35 (m, 2H), 3.75-3.64 (m, 1H), 3.62-3.41 (m, 10H), 3.35-3.15 (m, 2H), 2.07-1.87 (m, 6H), 1.53 (q, J=7.1 Hz, 2H), 1.47-1.33 (m, 2H), 0.80 (s, 1H), 0.46 (d, J=7.7 Hz, 2H), 0.14 (d, J=5.0 Hz, 2H). LC/MS (ESI, m/z): [(M+1)]⁺=575.36

### Step 4: 4-(15-[4-[(4-[2-[(2-Cyclopropylethyl)amino]-7-[trans-4-hydroxycyclohexyl]-7H-pyrrolo[2,3-d]pyrimidin-5-yl]phenyl)methyl]piperazin-1-yl]-4,7,10,13-tetraoxa-1-azapentadecan-1-yl)-2-(2,6-dioxopiperidin-3-yl)-2,3-dihydro-1H-isoindole-1,3-dione formate

To a stirred solution of trans-4-[2-[(2-cyclopropylethyl)amino]-5-[4-[(piperazin-1-yl)methyl]phenyl]-7H-pyrrolo[2,3-d]pyrimidin-7-yl]cyclohexan-1-ol (50 mg, 0.11 mmol) in DCM (5 mL) were added 14-[[2-(2,6-dioxopiperidin-3-yl)-1,3-dioxo-2,3-dihydro-1H-isoindol-4-yl]amino]-3,6,9,12-tetraoxatetradecanal (52 mg, 0.11 mmol), KOAc (41 mg, 0.42 mmol) and NaBH(OAc)₃ (67 mg, 0.32 mmol) at room temperature. After stirring for an additional 16 h at room temperature, the resulting mixture was concentrated under reduced pressure and the residue was purified by reverse phase flash chromatography with the following conditions: Column: WelFlash™ C18-I, 20-40 μm, 80 g; Eluent A: Water (plus 10 mmol/L HCOOH); Eluent B: ACN; Gradient: 45%-65% B in 20 min; Flow rate: 45 mL/min; Detector: 220/254 nm; desired fractions were collected at 62% B and concentrated under reduced pressure to afford 4-(15-[4-[(4-[2-[(2-cyclopropylethyl)amino]-7-[trans-4-hydroxycyclohexyl]-7H-pyrrolo[2,3-d]pyrimidin-5-yl]phenyl)methyl]piperazin-1-yl]-4,7,10,13-tetraoxa-1-azapentadecan-1-yl)-2-(2,6-dioxopiperidin-3-yl)-2,3-dihydro-1H-isoindole-1,3-dione formate as a yellow solid (13.7 mg, 14%). ¹H NMR (400 MHz, CD₃OD) δ 8.71 (s, 1H), 8.54 (s, 1H), 7.61 (d, J=8.0 Hz, 2H), 7.57-7.48 (m, 1H), 7.44-7.35 (m, 3H), 7.03 (dd, J=7.9, 3.5 Hz, 2H), 5.04 (dd, J=12.4, 5.4 Hz, 1H), 4.58-4.50 (m, 1H), 3.77-3.68 (m, 3H), 3.69-3.63 (m, 4H), 3.62 (s, 12H), 3.54 (t, J=7.2 Hz, 2H), 3.44 (t, J=5.2 Hz, 2H), 3.06-3.90 (m, 5H), 2.90-2.63 (m, 7H), 2.15-2.00 (m, 7H), 1.63-1.47 (m, 4H), 0.87-0.76 (m, 1H), 0.55-0.44 (m, 2H), 0.19-0.11 (m, 2H). LC/MS (ESI, m/z): [(M+1)]⁺=950.51

### EXAMPLE 10: 2-(2,6-Dioxopiperidin-3-yl)-4-[15-(4-[[4-(2-[[(4-methylphenyl)methyl]amino]-7-[trans-4-hydroxycyclohexyl]-7H-pyrrolo[2,3-d]pyrimidin-5-yl)phenyl]methyl] piperazin-1-yl)-4,7,10,13-tetraoxa-1-azapentadecan-1-yl]-2,3-dihydro-1H-isoindole-1,3-dione formate (I-26)

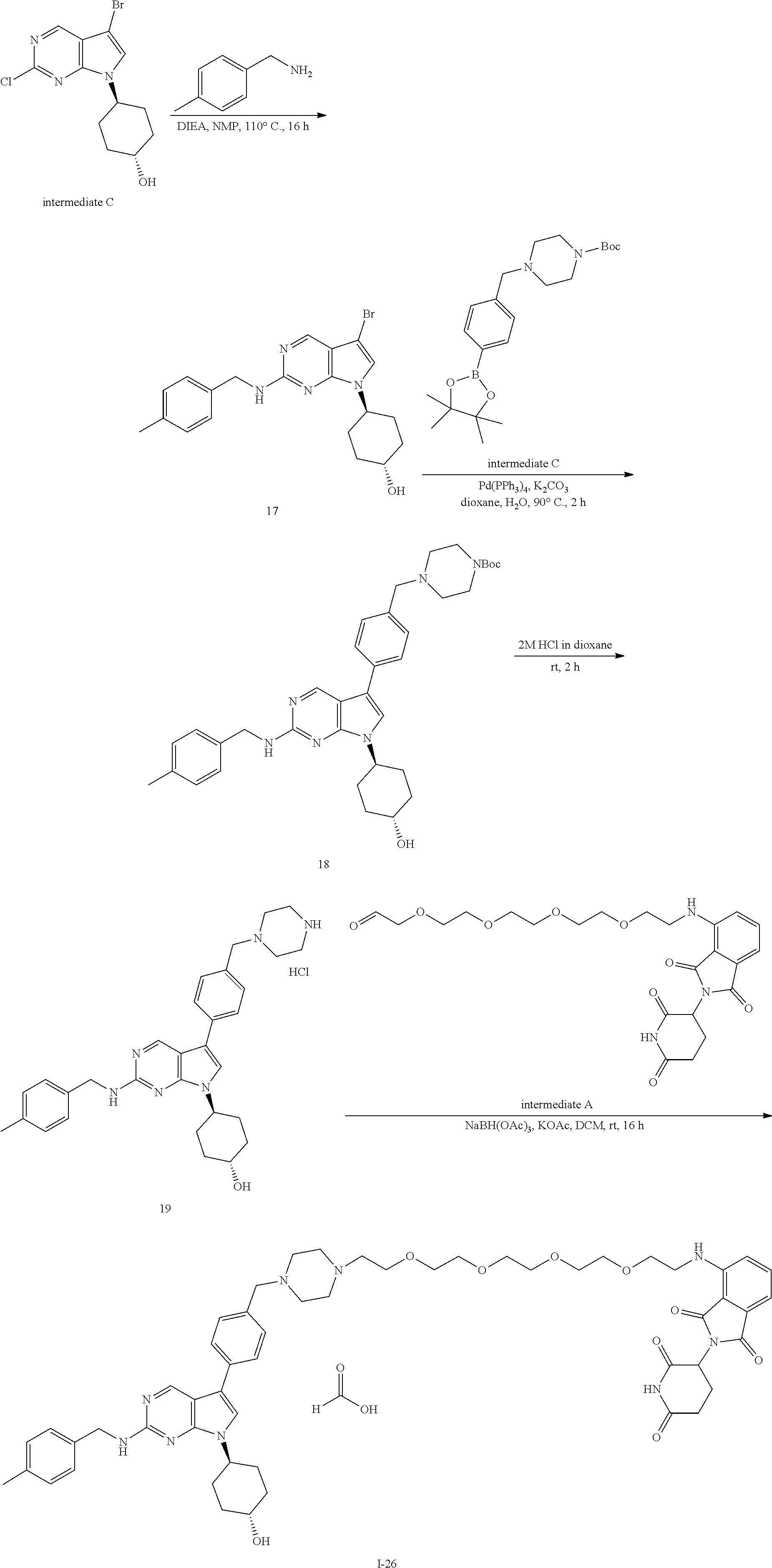

### Step 1: trans-4-(5-Bromo-2-[[(4-methylphenyl)methy]amino]-7H-pyrrolo[2,3-d]pyrimidin-7-yl)cyclohexan-1-ol

To a stirred solution of trans-4-[5-bromo-2-chloro-7H-pyrrolo[2,3-d]pyrimidin-7-yl]cyclohexan-1-ol (1 g, 3.03 mmol) in DMA (20 mL) were added DIEA (975 mg, 7.56 mmol) and 1-(4-methylphenyl)methanamine (733 mg, 6.05 mmol) at room temperature. The resulting solution was stirred at 110° C. for 16 h. The resulting mixture was cooled to room temperature and purified by reverse phase flash chromatography with the following conditions: Column: WelFlash™ C18-I, 20-40 μ.m, 330 g; Eluent A: Water (plus 10 mmol/L NH₄HCO₃); Eluent B: ACN; Gradient: 45%-65% B in 20 min; Flow rate: 80 mL/min; Detector: 220/254 nm; desired fractions were collected at 56% B and concentrated under reduced pressure to afford trans-4-(5-bromo-2-[[(4-methylphenyl)methyl]amino]-7H-pyrrolo[2,3-d]pyrimidin-7-yl)cyclohexan-1-ol (560 mg, 45%) as an orange solid. ¹H NMR (400 MHz, DMSO-d₆) δ 8.35 (s, 1H), 7.58 (s, 1H), 7.36 (s, 1H), 7.25 (d, J=7.7 Hz, 2H), 7.08 (d, J=7.8 Hz, 2H), 4.68 (d, J=4.5 Hz, 1H), 4.44 (d, J=6.2 Hz, 2H), 4.42-4.28 (m, 1H), 3.50 (dq, J=10.9, 5.8 Hz, 1H), 2.25 (s, 3H), 2.01-1.71 (m, 6H), 1.46-1.28 (m, 2H). LC/MS (ESI, m/z): [(M+1)]⁺=415.11, 417.11

### Step 2: tert-Butyl 4-[[4-(2-[[(4-methylphenyl)methy]amino]-7-[trans-4-hydroxycyclohexyl]-7H-pyrrolo[2,3-d]pyrimidin-5-yl)phenyl]methyl]piperazine-1-carboxylate

To a stirred solution of trans-4-(5-bromo-2-[[(4-methylphenyl)methyl]amino]-7H-pyrrolo[2,3-d]pyrimidin-7-yl)cyclohexan-1-ol (560 mg, 1.35 mmol) in 1,4-dioxane (5.6 mL) were added H₂O (2 mL), tert-butyl 4-[[4-(4,4,5,5-tetramethyl-1,3,2-dioxaborolan-2-yl)phenyl]methyl]piperazine-1-carboxylate (813 mg, 2.02 mmol), K₂CO₃ (279 mg, 2.02 mmol) and Pd(PPh₃)₄ (77.90 mg, 0.067 mmol) at room temperature under nitrogen atmosphere. The mixture was stirred at 90° C. for 2 h under nitrogen atmosphere. The resulting mixture was cooled down to room temperature, diluted with water (10 mL) and extracted with EtOAc (3×10 mL). The combined organic layers was washed with brine (5 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 20 min; Flow rate: 80 mL/min; Detector: 220/254 nm; desired fractions were collected at 53% B and concentrated under reduced pressure to afford tert-butyl 4-[[4-(2-[[(4-methylphenyl)methyl]amino]-7-[trans-4-hydroxycyclohexyl]-7H-pyrrolo[2,3-d]pyrimidin-5-yl)phenyl]methyl]piperazine-1-carboxylate (400 mg, 49%) as a yellow solid. ¹H NMR (400 MHz, DMSO-d₆) δ 8.82 (s, 1H), 7.64-7.59 (m, 2H), 7.57 (s, 1H), 7.43 (t, J=6.2 Hz, 1H), 7.34-7.26 (m, 4H), 7.09 (d, J=7.8 Hz, 2H), 4.69 (d, J=4.5 Hz, 1H), 4.47 (d, J=6.3 Hz, 2H), 4.44-4.34 (m, 1H), 3.59-3.50 (m, 1H), 3.48 (s, 2H), 3.32 (t, J=5.0 Hz, 4H), 2.33 (t, J=5.0 Hz, 4H), 2.25 (s, 3H), 2.03-1.77 (m, 6H), 1.45-1.32 (s, 11H). LC/MS (ESI, m/z): [(M+1)]⁺=611.50

### Step 3: trans-4-(2-[[(4-methylphenyl)methyl]amino]-5-[4-[(piperazin-1-yl)methyl]phenyl]-7H-pyrrolo[2,3-d]pyrimidin-7-yl)cyclohexan-1-ol

To a stirred solution of tert-butyl 4-[[4-(2-[[(4-methylphenyl)methyl]amino]-7-[trans-4-hydroxycyclohexyl]-7H-pyrrolo [2,3-d]pyrimidin-5-yl)phenyl]methyl]piperazine-1-carboxylate (390 mg, 0.64 mmol) in dioxane (5 mL) was added a solution of hydrochloride in 1,4-dioxane (4 M, 5 mL) at room temperature. The reaction was stirred at room temperature for 2 h. The resulting mixture was concentrated under reduced pressure to afford trans-4-(2-[[(4-methylphenyl)methyl]amino]-5-[4-[(piperazin-1-yl)methyl]phenyl]-7H-pyrrolo[2,3-d]pyrimidin-7-yl)cyclohexan-1-ol (300 mg, 92%) as a yellow solid. ¹H (400 MHz, DMSO-d₆) δ 9.75 (br s, 2H), 9.12 (s, 1H), 8.60 (s, 1H), 8.15 (s, 1H), 7.82 (d, J=7.9 Hz, 2H), 7.73 (d, J=7.9 Hz, 2H), 7.35 (d, J=7.6 Hz, 2H), 7.15 (d, J=7.7 Hz, 2H), 4.61 (s, 2H), 4.51-4.38 (m, 3H), 3.60-3.41 (m, 6H), 3.27 (s, 3H), 2.28 (s, 3H), 2.04-1.75 (m, 6H), 1.48-1.34 (m, 2H). LC/MS (ESI, m/z): [(M+1)]⁺=511.45

### Step 4: 2-(2,6-Dioxopiperidin-3-yl)-4-[15-(4-[[4-(2-[[(4-methylphenyl)methyl]amino]-7-[trans-4-hydroxycyclohexyl]-7H-pyrrolo[2,3-d]pyrimidin-5-yl)pheny]methyl]piperazin-1-yl)-4,7,10,13-tetraoxa-1-azapentadecan-1-yl]-2,3-dihydro-1H-isoindole-1,3-dioneformate

To a stirred solution of trans-4-(2-[[(4-methylphenyl)methyl]amino]-5-[4-[(piperazin-1-yl)methyl]phenyl]-7H-pyrrolo[2,3-d]pyrimidin-7-yl)cyclohexan-1-ol (50 mg, 0.098 mmol) in DCM (5 mL) were added 14-[[2-(2,6-dioxopiperidin-3-yl)-1,3-dioxo-2,3-dihydro-1H-isoindol-4-yl]amino]-3,6,9,12-tetraoxatetradecanal (48 mg, 0.098 mmol), KOAc (38 mg, 0.39 mmol) and NaBH(AcO)₃ (62 mg, 0.29 mmol) at room temperature. The resulting mixture was stirred for an additional 16 h at room temperature. The resulting mixture was concentrated under reduced pressure. The residue was purified by reverse phase flash chromatography with the following conditions: Column: WelFlash™ C18-I, 20-40 μm, 80 g; Eluent A: Water (plus 10 mmol/L HCOOH); Eluent B: ACN; Gradient: 45%-65% B in 20 min; Flow rate: 45 mL/min; Detector: 220/254 nm; desired fractions were collected at 56% B and concentrated under reduced pressure to afford 2-(2,6-dioxopiperidin-3-yl)-4-[15-(4-[[4-(2-[[(4-methylphenyl)methyl]amino]-7-[trans-4-hydroxycyclohexyl]-7H-pyrrolo [2,3-d]pyrimidin-5-yl)phenyl]methyl]piperazin-1-yl)-4,7,10,13-tetraoxa-1-azapentadecan-1-yl]-2,3-dihydro-1H-isoindole-1,3-dione formate (10 mg, 10%) as a yellow solid. ¹H NMR (400 MHz, DMSO-d₆) δ 11.12 (br s, 1H), 8.81 (s, 1H), 8.17 (s, 1H), 7.65-7.53 (m, 4H), 7.50-7.40 (m, 1H), 7.32-7.20 (m, 4H), 7.18-7.00 (m, 4H), 6.60 (s, 1H), 5.06 (d, J=8.2 Hz, 1H), 4.70 (s, 1H), 4.46 (s, 2H), 4.45-4.34 (m, 1H), 3.65-3.40 (m, 21H), 2.95-2.82 (s, 1H), 2.60-2.30 (m, 10H), 2.25 (s, 3H), 2.07-1.75 (m, 8H), 1.37 (d, J=12.9 Hz, 2H). LC/MS (ESI, m/z): [(M+1)]⁺=987.05

### EXAMPLE 11: 4-[15-[4-([6-[2-(Butylamino)-4-[[trans-4-hydroxycyclohexyl]amino]pyrimidin-5-yl]pyridin-3-yl]methyl)piperazin-1-yl]-4,7,10,13-tetraoxa-1-azapentadecan-1-yl]-2-(2,6-dioxopiperidin-3-yl)-2,3-dihydro-1H-isoindole-1,3-dione (I-25)

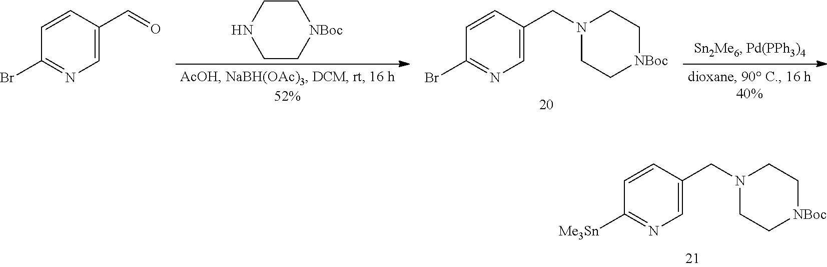

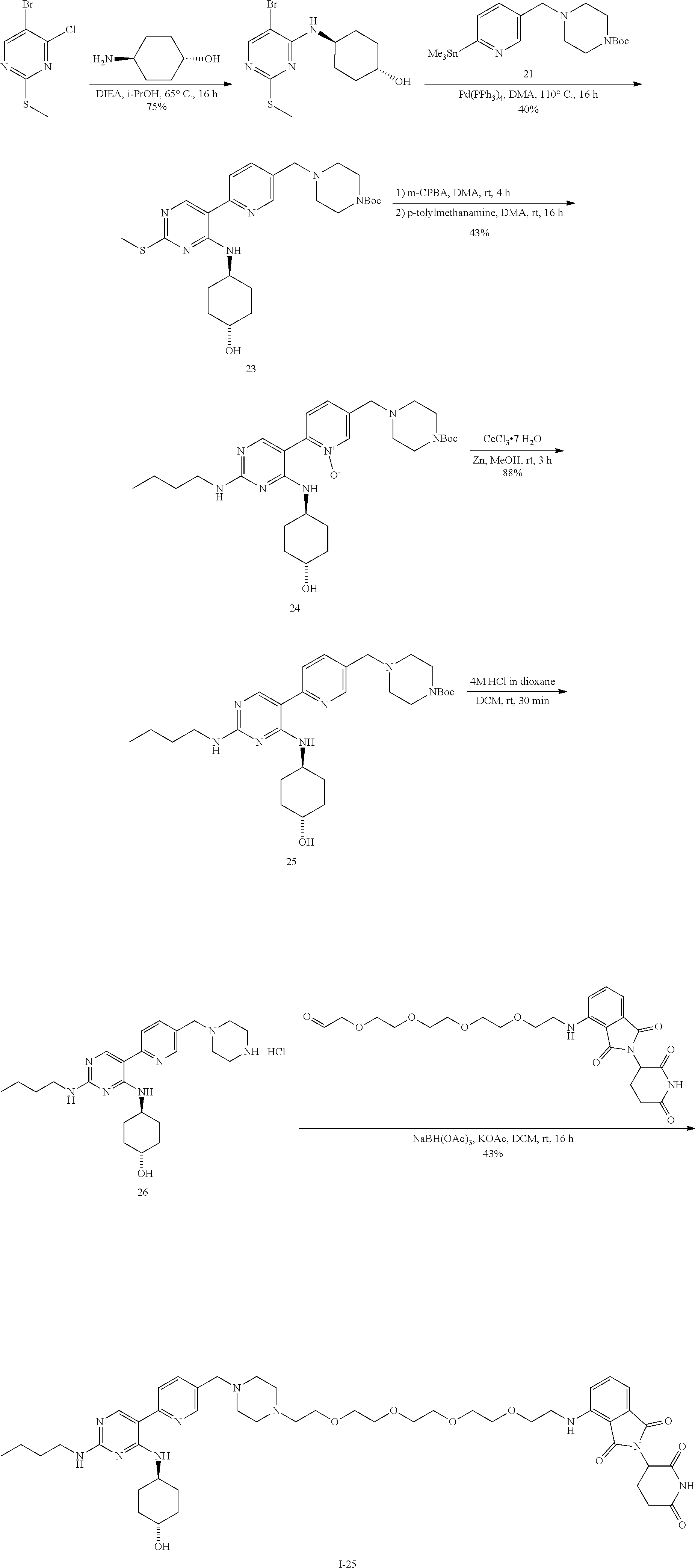

### Step 1: tert-Butyl 4-[(6-bromopyridin-3-yl)methy]piperazine-1-carboxylate

To a stirred solution of 6-bromopyridine-3-carbaldehyde (5 g, 26.88 mmol) in DCM (100 mL) were added AcOH (1.54 mL, 25.65 mmol), tert-butyl piperazine-1-carboxylate (5.51 g, 29.57 mmol) and NaBH(OAc)₃ (17.09 g, 80.64 mmol) at 0° C. under nitrogen atmosphere. The resulting mixture was stirred for 16 h at room temperature under nitrogen atmosphere. The resulting mixture was diluted with saturated aqueous solution of NaHCO₃ (100 mL) and extracted with DCM (3×100 mL). The combined organic layers was washed with brine (100 mL) and dried over anhydrous sodium sulfate. After filtration, the filtrate was concentrated under reduced pressure. The residue was triturated with Et₂O (10 mL) and collected after filtration to afford tert-butyl 4-[(6-bromopyridin-3-yl)methyl]piperazine-1-carboxylate (5 g, 52%) as a light yellow solid. ¹H NMR (400 MHz, DMSO-d₆) δ 8.31 (s, 1H), 7.69 (d, J=8.2 Hz, 1H), 7.62 (d, J=8.0 Hz, 1H), 3.50 (s, 2H), 3.31 (s, 4H), 2.32 (s, 4H), 1.38 (s, 9H). LC/MS (ESI, m/z): [(M+1)]⁺=356.00, 358.00

### Step 2: tert-Butyl 4-[[6-(trimethylstannyl)pyridin-3-y]methyl]piperazine-1-carboxylate

To a solution of tert-butyl 4-[(6-bromopyridin-3-yl)methyl]piperazine-1-carboxylate (200 mg, 0.56 mmol) in dioxane (3 mL) were added Sn₂Me₆ (367 mg, 1.12 mmol) and Pd(PPh₃)₄ (64 mg, 0.056 mmol). The resulting solution was stirred for 16 h at 90° C. under nitrogen atmosphere. The resulting solution was cooled to room temperature and diluted with water (50 mL). The mixture was extracted with EtOAc (3×50 mL). The combined organic phase was washed with brine (20 mL), dried over anhydrous Na₂SO₄ and filtered. The filtrate was concentrated under reduced pressure. The residue was purified by reverse phase flash chromatography with the following conditions: Column: WelFlash™ C18-I, 20-40 μm, 120 g; Eluent A: Water (plus 10 mmol/L NH₄HCO₃); Eluent B: ACN; Gradient: 50%-70% B in 25 min; Flow rate: 45 mL/min; Detector: 220/254 nm; desired fractions were collected at 66% B and concentrated under reduced pressure to afford tert-butyl 4-[[6-(trimethylstannyl)pyridin-3-yl]methyl]piperazine-1-carboxylate (100 mg, 40%) as a light yellow solid. ¹H NMR (400 MHz, DMSO-d₆) δ 8.60 (dd, J=2.2, 1.0 Hz, 1H), 7.60-7.46 (m, 2H), 3.46 (s, 2H), 3.30 (s, 4H), 2.31 (s, 4H), 1.39 (s, 9H), 0.28 (s, 9H). LC/MS (ESI, m/z): [(M+1)]⁺=438.1, 440.1, 442.1

### Step 3: trans-4-[[5-Bromo-2-(methylsulfanyl)pyrimidin-4-yl]amino]cyclohexan-1-ol

To a solution of 5-bromo-4-chloro-2-(methylsulfanyl)pyrimidine (900 mg, 3.76 mmol) in i-PrOH (15 mL) were added DIEA (1.31 mL, 10.13 mmol) and trans-4-aminocyclohexan-1-ol (0.43 g, 3.76 mmol). The resulting solution was stirred for 16 h at 65° C. under nitrogen atmosphere. After cooling down to room temperature, the resulting solution was diluted with water (100 mL) and extracted with DCM (3×30 mL). The combined organic phase was washed with brine (100 mL) and dried over anhydrous sodium sulfate. After filtration, the filtrate was concentrated under reduced pressure. The residue was purified by silica gel column chromatography, eluted with 10% methanol in dichloromethane to afford trans-4-[[5-bromo-2-(methyl sulfanyl)pyrimidin-4-yl]amino]cyclohexan-1-ol (900 mg, 75%) as a yellow solid. ¹H NMR (400 MHz, DMSO-d₆) δ 8.12 (d, J=1.3 Hz, 1H), 6.78 (d, J=7.9 Hz, 1H), 4.58 (d, J=4.3 Hz, 1H), 3.90 (dtt, J=11.5, 7.7, 3.9 Hz, 1H), 3.50-3.35 (m, 1H), 2.42 (d, J=1.3 Hz, 3H), 1.89-1.76 (m, 4H), 1.56-1.41 (m, 2H), 1.30-1.16 (m, 2H). LC/MS (ESI, m/z): [(M+1)]⁺=318.15, 320.15

### Step 4: tert-Butyl 4-[(6-[4-[(4-hydroxycyclohexyl)amino]-2-(methylsulfanyl)pyrimidin-5-yl]pyridin-3-yl)methyl]piperazine-1-carboxylate

To a solution of trans-4-[[5-bromo-2-(methylsulfanyl)pyrimidin-4-yl]amino]cyclohexan-1-ol (4.6 g, 14.5 mmol) in DMA (50 mL) were added tert-butyl 4-[[6-(trimethylstannyl)pyridin-3-yl]methyl]piperazine-1-carboxylate (6.4 g, 14.5 mmol) and Pd(PPh₃)₄ (1.7 g, 1.5 mmol). The resulting solution was stirred for 16 h at 110° C. under nitrogen atmosphere. The resulting mixture was cooled down to room temperature and purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 30%-50% B in 20 min; Flow rate: 80 mL/min; Detector: 220/254 nm; desired fractions were collected at 43% B and concentrated under reduced pressure to afford tert-butyl 4-[(6-[4-[(4-hydroxycyclohexyl)amino]-2-(methylsulfanyl)pyrimidin-5-yl]pyridin-3-yl)methyl]piperazine-1-carboxylate (3 g, 40%) as a yellow solid. ¹H NMR (400 MHz, DMSO-d₆) δ 10.06 (d, J=7.2 Hz, 1H), 8.69 (s, 1H), 8.52 (s, 1H), 8.05 (d, J=8.4 Hz, 1H), 7.82 (d, J=8.4 Hz, 1H), 4.58 (d, J=4.2 Hz, 1H), 4.05-3.93 (m, 1H), 3.59-3.47 (m, 3H), 2.55 (s, 4H), 2.49 (s, 3H), 2.40-2.26 (m, 4H), 2.11-1.98 (m, 2H), 1.91-1.81 (m, 2H), 1.39 (s, 9H), 1.38-1.27 (m, 4H). LC/MS (ESI, m/z): [(M+1)]⁺=515.25

### Step 5: 5-((4-(tert-butoxycarbonyl)piperazin-1-yl)methyl)-2-(2-(butylamino)-4-(((1r,4r)-4-hydroxycyclohexyl)amino)pyrimidin-5-yl)pyridine 1-oxide

To a stirred solution of tert-butyl 4-[(6-[4-[(4-hydroxycyclohexyl)amino]-2-(methylsulfanyl)pyrimidin-5-yl]pyridin-3-yl)methyl]piperazine-1-carboxyl ate (600 mg, 1.17 mmol) in DMA (15 mL) was added m-CPBA (1.01 g, 5.83 mmol) at 0° C. under nitrogen atmosphere. The resulting solution was stirred for 3 h at room temperature. Then butan-1-amine (853 mg, 11.66 mmol) was added to the reaction. The resulting solution was stirred at 50° C. for 3 h. The resulting solution was cooled to room temperature and purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 20 min; Flow rate: 80 mL/min; Detector: 220/254 nm; desired fractions were collected at 53% B and concentrated under reduced pressure to afford 5-([4-[(tert-butoxy)carbonyl]piperazin-1-yl]methyl)-2-[2-(butylamino)-4-[(4-hydroxycyclohexyl)amino]pyrimidin-5-yl]pyridin-1-ium-1-olate (350 mg, 54%) as an off white solid. ¹H NMR (400 MHz, DMSO-d₆) δ 9.91 (s, 1H), 8.65-8.48 (m, 2H), 8.01-7.84 (m, 2H), 7.05 (s, 1H), 4.60 (d, J=4.3 Hz, 1H), 4.31 (s, 2H), 4.00-3.87 (m, 1H), 3.84-3.67 (m, 2H), 3.59-3.43 (m, 2H), 3.33-3.20 (m, 6H), 2.78-2.63 (m, 1H), 2.13-1.97 (m, 2H), 1.93-1.79 (m, 2H), 1.61-1.48 (m, 2H), 1.41 (s, 9H), 1.38-1.21 (m, 6H), 0.91 (t, J=7.1 Hz, 3H). LC/MS (ESI, m/z): [(M+1)]⁺=556.35

### Step 6: tert-Butyl 4-([6-[2-(butylamino)-4-[[trans-4-hydroxycyclohexyl]amino]pyrimidin-5-yl]pyridin-3-yl]methyl)piperazine-1-carboxylate

To a stirred solution of 5-([4-[(tert-butoxy)carbonyl]piperazin-1-yl]methyl)-2-[2-(butylamino)-4-[[trans-4-hydroxycyclohexyl]amino]pyrimidin-5-yl]pyridin-1-ium-1-olate (350 mg, 0.63 mmol) in MeOH (10 mL) were added CeCl₃.7 H₂O (1173 mg, 3.15 mmol) and zinc dust (1236 mg, 18.89 mmol) at room temperature under nitrogen atmosphere. After stirring for an additional 3 h at room temperature, the resulting mixture was filtered and the filtrate was concentrated under reduced pressure. The residue was purified by silica gel column chromatography, eluted with 10% methanol in dichloromethane to afford tert-butyl 4-([6-[2-(butylamino)-4-[[trans-4-hydroxycyclohexyl]amino]pyrimidin-5-yl]pyridin-3-yl]methyl)piperazine-1-carboxylate (300 mg, 88%) as an off white solid. ¹H NMR (400 MHz, DMSO-d₆) δ 10.43 (d, J=7.2 Hz, 1H), 8.48 (d, J=2.2 Hz, 1H), 8.36 (s, 1H), 7.94 (d, J=8.4 Hz, 1H), 7.78 (dd, J=8.4, 2.2 Hz, 1H), 6.86 (t, J=6.1 Hz, 1H), 4.67-4.58 (m, 1H), 3.95 (m, 1H), 3.53 (s, 2H), 3.47-3.52 (m, 1H), 3.43 (q, J=6.7 Hz, 2H), 3.32 (m, 4H), 2.34 (m, 4H), 2.05 (m, 4H), 1.87 (d, J=11.9 Hz, 2H), 1.58 (m, 2H), 1.39 (s, 9H), 1.38-1.32 (m, 4H), 0.94 (t, J=7.4 Hz, 3H). LC/MS (ESI, m/z): [(M+1)]⁺=540.40

### Step 7: trans-4-[[2-(Butylamino)-5-[5-[(piperazin-1-yl)methyl]pyridin-2-yl]pyrimidin-4-yl]amino]cyclohexan-1-ol

To a stirred solution of tert-butyl 4-([6-[2-(butylamino)-4-[[trans-4-hydroxycyclohexyl]amino]pyrimidin-5-yl]pyridin-3-yl]methyl)piperazine-1-carboxylate (300 mg, 0.56 mmol) in DCM (5 mL) was added a solution of hydrochloride in 1,4-dioxane (4 M, 5 mL) at room temperature under nitrogen atmosphere. The solution was stirred for 30 min at room temperature. The solution was concentrated under reduced pressure to afford trans-4-[[2-(butylamino)-5-[5-[(piperazin-1-yl)methyl]pyridin-2-yl]pyrimidin-4-yl]amino]cyclohexan-1-ol hydrochloride (350 mg, crude) as a colorless oil. ^{I}H NMR (400 MHz, D₂O) δ 8.66 (d, J=2.2 Hz, 1H), 8.05 (m, 2H), 7.77 (d, J=8.4 Hz, 1H), 4.51 (s, 2H), 4.07 (m, 1H), 3.72-3.28 (m, 11H), 2.12-1.87 (m, 4H), 1.56 (m, 2H), 1.49-1.24 (m, 6H), 0.87 (t, J=7.4 Hz, 3H). LC/MS (ESI, m/z): [(M +1)]⁺=440.30

### Step 8: 4-[15-[4-([6-[2-(Butylamino)-4-[[trans-4-hydroxycyclohexyl]amino]pyrimidin-5-yl]pyridin-3-yl]methyl)piperazin-1-yl]-4,7,10,13-tetraoxa-1-azapentadecan-1-yl]-2-(2,6-dioxopiperidin-3-yl)-2,3-dihydro-1H-isoindole-1,3-dione

To a stirred solution of trans-4-[[2-(butylamino)-5-[5-[(piperazin-1-yl)methyl]pyridin-2-yl]pyrimidin-4-yl]amino]cyclohexan-1-ol hydrochloride (300 mg, 0.63 mmol) in DCM (20 mL) were added 14-[[2-(2,6-dioxopiperidin-3-yl)-1,3-dioxo-2,3-dihydro-1H-isoindol-4-yl]amino]-3,6,9,12-tetraoxatetradecanal (372 mg, 0.76 mmol), KOAc (247 mg, 2.52 mmol) and NaBH₃CN (534 mg, 2.52 mmol) at room temperature. The resulting mixture was stirred for an additional 4 h at room temperature. The resulting solution was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 52% B and concentrated under reduced pressure to afford 4-[15-[4-([6-[2-(butylamino)-4-[[trans-4-hydroxycyclohexyl]amino]pyrimidin-5-yl]pyridin-3-yl]methyl)piperazin-1-yl]-4,7,10,13-tetraoxa-1-azapentadecan-1-yl]-2-(2,6-dioxopiperidin-3-yl)-2,3-dihydro-1H-isoindole-1,3-dione (250 mg, 43%) as a light yellow solid. ¹H NMR (400 MHz, Methanol-d₄) δ 8.57 (d, J=2.1 Hz, 1H), 8.30 (s, 1H), 7.87 (q, J=8.5 Hz, 2H), 7.53 (t, J=7.8 Hz, 1H), 7.11-6.99 (m, 2H), 5.07 (dd, J=12.7, 5.5 Hz, 1H), 4.14 (s, 1H), 3.83 (t, J=4.8 Hz, 2H), 3.76-3.59 (m, 18H), 3.56-3.41 (m, 5H), 3.40-3.30 (m, 3H), 3.17-2.37 (m, 8H), 2.27-1.98 (m, 5H), 1.75-1.65 (m, 2H), 1.62-1.40 (m, 6H), 1.03 (t, J=7.4 Hz, 3H). LC/MS (ESI, m/z): [(M+1)]³⁰ =915.35

### EXAMPLE 12: 2-(2,6-Dioxopiperidin-3-yl)-4-[15-(4-[[6-(2-[[(4-methylphenyl)methyl]amino]-4-[[trans-4-hydroxycyclohexyl]amino]pyrimidin-5-yl)pyridin-3-yl]methyl]piperazin-1-yl)-4,7,10,13-tetraoxa-1-azapentadecan-1-yl]-2,3-dihydro-1H-isoindole-1,3-dione (I-21)

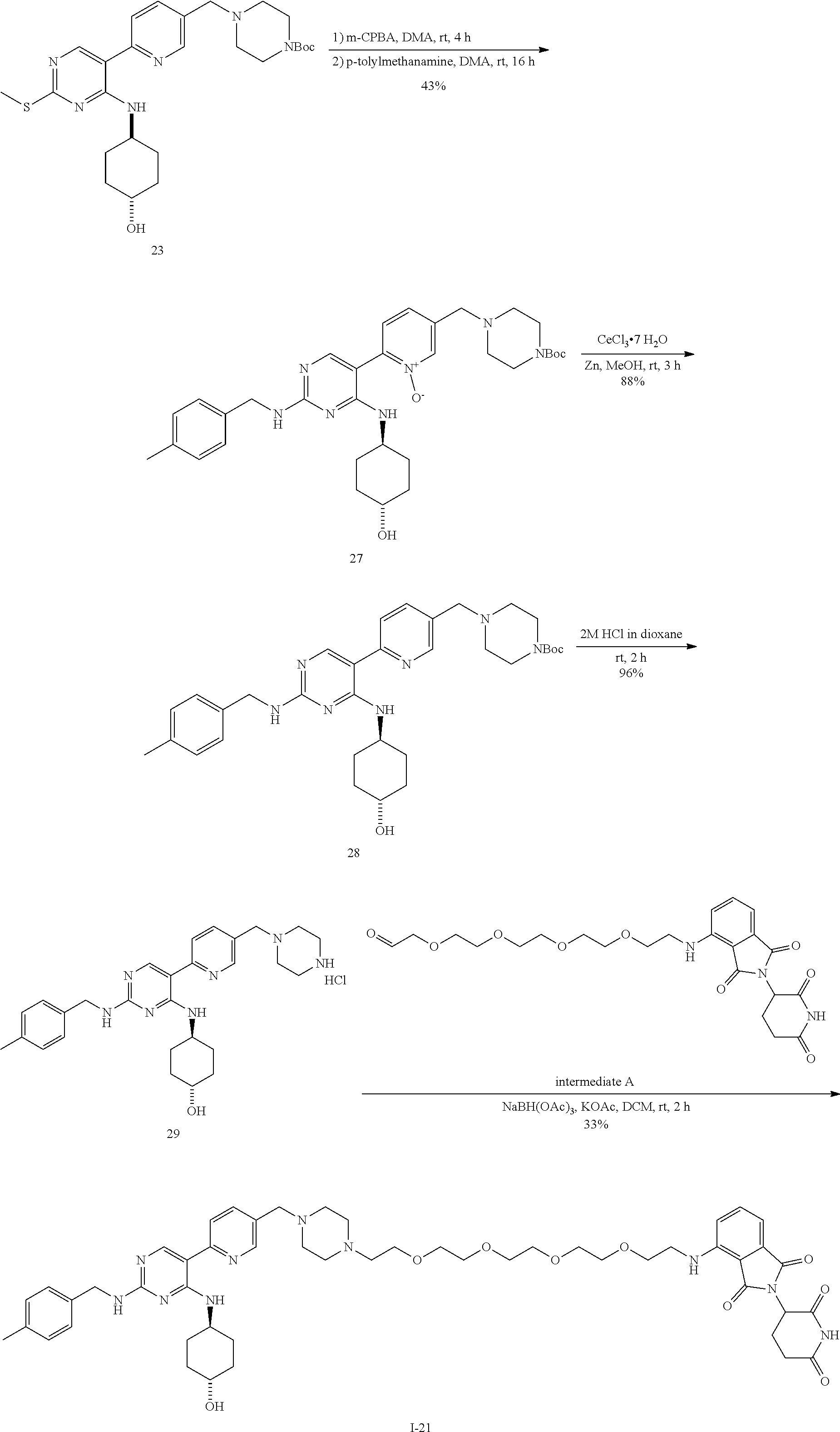

### Step 1: 5-((4-(tert-Butoxycarbonyl)piperazin-1-yl)methyl)-2-(4-(trans-4-hydroxycyclohexylamino)-2-(4-methylbenzylamino)pyrimidin-5-yl)pyridine 1-oxide

To a stirred solution of tert-butyl 4-[(6-[4-[(trans-4-hydroxycyclohexyl)amino]-2-(methylsulfanyl)pyrimidin-5-yl]pyridin-3-yl)methyl]piperazine-1-carboxylate (300 mg, 0.58 mmol) in DMA (5 mL) was added m-CPBA (302 mg, 1.75 mmol) at room temperature under nitrogen atmosphere. After stirring for an additional 4 h at room temperature, 1-(4-methylphenyl) methanamine (565 mg, 4.66 mmol) was added. The resulting mixture was stirred for an additional 16 h at room temperature. The resulting mixture was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 30%-50% B in 25 min; Flow rate: 80 mL/min; Detector: 220/254 nm; desired fractions were collected at 48% B and concentrated under reduced pressure to afford 5-((4-(tert-butoxycarbonyl)piperazin-1-yl)methyl)-2-(4-(trans-4-hydroxycyclohexylamino)-2-(4-methylbenzylamino)pyrimidin-5-yl)pyridine 1-oxide (150 mg, 43%) as a yellow solid. ¹H NMR (400 MHz, DMSO-d₆) δ 9.88 (d, J=7.5 Hz, 1H), 8.55 (m, 2H), 7.99-7.83 (m, 2H), 7.69-7.46 (m, 1H), 7.17 (m, 4H), 4.64-4.51 (m, 1H), 4.49-4.38 (m, 2H), 4.31 (s, 1H), 4.00-3.58 (m, 4H), 3.57-3.42 (m, 1H), 2.75-2.61 (m, 4H), 2.38-2.20 (m, 5H), 2.01-1.75 (m, 4H), 1.41 (s, 9H), 1.35-1.14 (m, 4H). LC/MS (ESI, m/z): [(M+1)]⁺=604.40

### Step2: tert-Butyl 4-[[6-(2-[[(4-methylphenyl)methyl]amino]-4-[[trans-4-hydroxycyclohexyl]amino]pyrimidin-5-yl)pyridin-3-yl]methyl]piperazine-1-carboxylate

To a stirred solution of 5-((4-(tert-butoxycarbonyl)piperazin-1-yl)methyl)-2-(4-(trans-4-hydroxycyclohexylamino)-2-(4-methylbenzylamino)pyrimidin-5-yl)pyridine 1-oxide (350 mg, 0.63 mmol) in MeOH (10 mL) were added CeCl₃.7H₂O (1173 mg, 3.15 mmol) and zinc dust (1236 mg, 18.89 mmol) at room temperature under nitrogen atmosphere. After stirring for an additional 3 h at room temperature, the resulting mixture was filtered and the filtrate was concentrated under reduced pressure. The residue was purified by silica gel column chromatography, eluted with 10% methanol in dichloromethane to afford 4-[[6-(2-[[(4-methylphenyl)methyl]amino]-4-[[trans-4-hydroxycyclohexyl]amino]pyrimidin-5-yl)pyridin-3-yl]methyl]piperazine-1-carboxylate (300 mg, 88%) as an off white solid. ¹H NMR (400 MHz, DMSO-d₆) δ 9.90 (s, 1H), 8.48 (s, 1H), 8.39 (s, 1H), 7.86 (d, J=8.5 Hz, 1H), 7.70 (d, J=8.7 Hz, 1H), 7.66-7.51 (m, 1H), 7.23 (d, J=7.8 Hz, 2H), 7.10 (d, J=7.8 Hz, 2H), 4.56 (d, J=4.4 Hz, 1H), 4.42 (s, 2H), 3.88 (s, 1H), 3.52 (s, 1H), 3.45 (s, 4H), 3.33-3.29 (m, 2H), 2.34 (s, 4H), 2.27 (s, 3H), 1.93 (s, 2H), 1.82 (s, 2H), 1.39 (s, 9H), 1.32-1.17 (m, 4H). LC/MS (ESI, m/z): [(M+=1)]⁺=588.45

### Step 3: trans-4-[(2-[[(4-methylphenyl)methyl]amino]-[5-[(piperazin-1-yl)methyl]pyridin-2-yl]pyrimidin-4-yl)amino]cyclohexan-1-ol

To a stirred solution of tert-butyl 4-[[6-(2-[[(4-methylphenyl)methyl]amino]-4-[[trans-4-hydroxycyclohexyl]amino]pyrimidin-5-yl)pyridin-3-yl]methyl]piperazine-1-carboxylate (100 mg, 0.17 mmol) in dioxane (10 mL) was added a solution of hydrochloride in 1,4-dioxane (4 M, 10 mL) at 0° C. under nitrogen atmosphere. The resulting solution was stirred for 2 h at room temperature and concentrated under reduced pressure to afford trans-4-[(2-[[(4-methylphenyl)methyl]amino]-5-[5-[(piperazin-1-yl)methyl]pyridin-2-yl]pyrimidin-4-yl)amino]cyclohexan-1-ol (80 mg, 96%) as a white solid. ¹H NMR (400 MHz, Methanol-d₄) δ 8.86 (s, 1H), 8.48 (s, 1H), 8.26 (d, J=8.5 Hz, 1H), 8.05 (d, J=8.2 Hz, 1H), 7.29 (d, J=7.8 Hz, 2H), 7.20 (d, J=7.8 Hz, 2H), 4.63-4.45 (m, 3H), 4.13-4.01 (m, 1H), 3.79-3.73 (m, 1H), 3.73-3.52 (m, 9H), 2.34 (s, 3H), 2.08-1.92 (m, 4H), 1.53-1.38 (m, 4H). LC/MS (ESI, m/z): [(M+1)]⁺=488.50

### Step 4: 2-(2,6-Dioxopiperidin-3-yl)-4-[15-(4-[[6-(2-[[(4-methylphenyl)methyl]amino]-4-[[trans-4-hydroxycyclohexyl]amino]pyrimidin-5-yl)pyridin-3-yl]methyl]piperazin-1-yl)-4,7,10,13-tetraoxa-1-azapentadecan-1-yl]-2,3-dihydro-1H-isoindole-1,3-dione

To a stirred solution of trans-4-[(2-[[(4-methylphenyl)methyl]amino]-5-[5-[(piperazin-1-yl)methyl]pyridin-2-yl]pyrimidin-4-yl)amino]cyclohexan-1-ol hydrochloride (50 mg, 0.095 mmol) in DCM (20 mL) were added 14-[[2-(2,6-dioxopiperidin-3-yl)-1,3-dioxo-2,3-dihydro-1H-isoindol-4-yl]amino]-3,6,9,12-tetraoxatetradecanal (47 mg, 0.095 mmol), KOAc (56 mg, 0.57 mmol) and NaBH(AcO)₃ (67 mg, 0.29 mmol) at room temperature under nitrogen atmosphere. The resulting mixture was stirred for 2 h at room temperature and was concentrated under reduced pressure. The residue was purified by reverse phase flash chromatography with the following conditions: Column: WelFlash™ C18-I, 20-40 μm, 80 g; Eluent A: Water (plus 10 mmol/L NH₄HCO₃); Eluent B: ACN; Gradient: 40%-60% B in 25 min; Flow rate: 45 mL/min; Detector: 220/254 nm; desired fractions were collected at 51% B and concentrated under reduced pressure to afford 2-(2,6-dioxopiperidin-3-yl)-4-[15-(4-[[6-(2-[[(4-methylphenyl)methyl]amino]-4-[[trans-4-hydroxycyclohexyl]amino]pyrimidin-5-yl)pyridin-3-yl]methyl]piperazin-1-yl)-4,7,10,13-tetraoxa-1-azapentadecan-1-yl]-2,3-dihydro-1H-isoindole-1,3-dione (30 mg, 33%) as an off light yellow solid. ¹H NMR (400 MHz, Methanol-d₄) δ 8.41 (s, 1H), 8.33 (s, 1H), 7.73 (s, 2H), 7.52 (s, 1H), 7.25 (d, J=7.8 Hz, 2H), 7.13 (d, J=7.6 Hz, 2H), 7.05 (d, J=7.4 Hz, 2H), 5.04 (dd, J=11.9, 5.4 Hz, 1H), 4.54 (s, 2H), 3.94 (s, 1H), 3.77-3.39 (m, 20H), 3.34 (m, 2H), 2.83-2.53 (m, 13H), 2.31 (s, 3H), 2.17-1.88 (m, 4H), 1.45-1.25 (m, 4H). LC/MS (ESI, m/z): [(M+1)]⁺=963.50

### EXAMPLE 13: 4-[15-[4-([6-[2-(Butylamino)-4-[[trans-4-hydroxycyclohexyl]amino]pyrimidin-5-yl]pyridin-3-yl]methyl)piperazin-1-yl]-15-oxo-4,7,10,13-tetraoxa-1-azapentadecan-1-yl]-2-(2,6-dioxopiperidin-3-yl)-2,3-dihydro-1H-isoindole-1,3-dione (I-22)

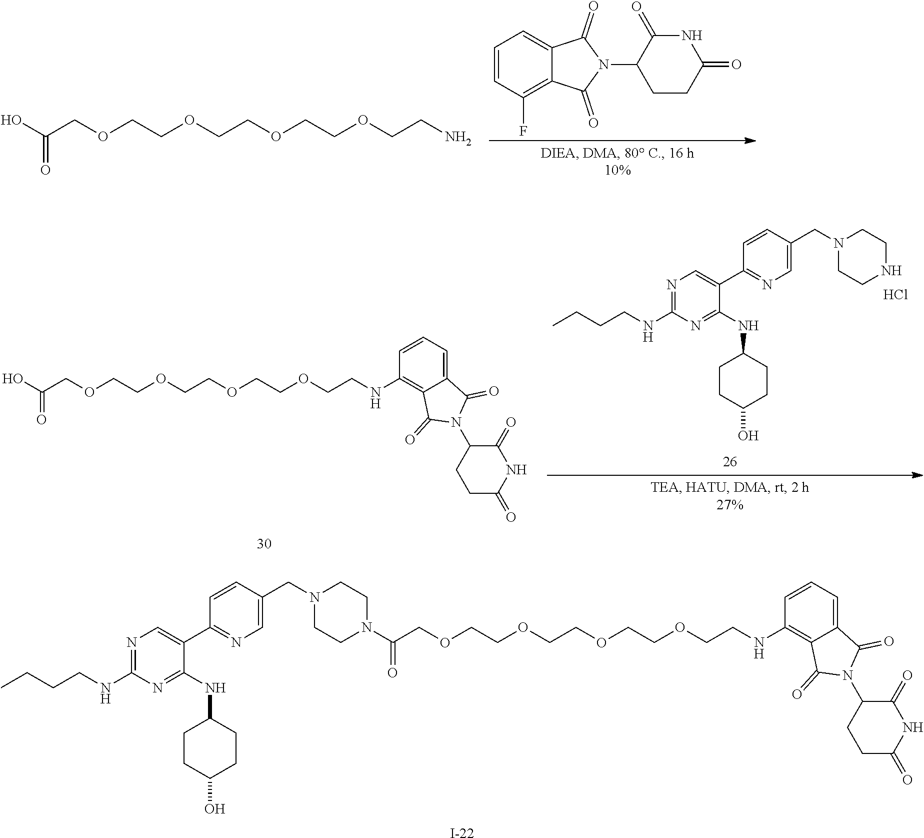

### Step 1: 14-[[2-(2,6-dioxopiperidin-3-yl)-1,3-dioxo-2,3-dihydro-1H-isoindol-4-yl]amino]-3,6,9,12-tetraoxatetradecanoic acid

To a solution of 14-amino-3,6,9,12-tetraoxatetradecanoic acid (400 mg, 1.59 mmol) in DMA (15 mL) were added 2-(2,6-dioxopiperidin-3-yl)-4-fluoro-2,3-dihydro-1H-isoindole-1,3-dione. (439 mg, 1.59 mmol) and DIEA (411 mg, 3.18 mmol) at room temperature. The resulting solution was stirred for 16 h at 80° C. under nitrogen atmosphere. After cooling down to room temperature, the mixture was purified by reverse phase flash chromatography with the following conditions: Column: WelFlash™ C18-I, 20-40 μm, 80 g; Eluent A: Water (plus 10 mmol/L NH₄HCO₃); Eluent B: ACN; Gradient: 40%-50% B in 20 min; Flow rate: 45 mL/min; Detector: 220/254 nm; desired fractions were collected at 46% B and concentrated under reduced pressure to afford 14-[[2-(2,6-dioxopiperidin-3-yl)-1,3-dioxo-2,3-dihydro-1H-isoindol-4-yl]amino]-3,6,9,12-tetraoxatetradecanoic acid (100 mg, 10%) as a yellow solid. ¹H NMR (400 MHz, Methanol-d₄) δ 7.63-7.50 (m, 1H), 7.18-7.03 (m, 2H), 5.07 (dd, J=12.6, 5.5 Hz, 1H), 4.12 (d, J=1.3 Hz, 2H), 3.89 (t, J=5.6 Hz, 1H), 3.79-3.73 (m, 2H), 3.71-3.62 (m, 10H), 3.62-3.59 (m, 1H), 3.54 (t, J=5.2 Hz, 2H), 2.98-2.63 (m, 3H), 2.22-2.07 (m, 1H). LC/MS (ESI, m/z): [(M+1)]⁺=508.15

### Step 2: 4-[15-[4-([6-[2-(Butylamino)-4-[[trans-4-hydroxycyclohexyl]amino]pyrimidin-5-yl]pyridin-3-yl]methyl)piperazin-1-yl]-15-oxo-4,7,10,13-tetraoxa-1-azapentadecan-1-yl]-2-(2,6-dioxopiperidin-3-yl)-2,3-dihydro-1H-isoindole-1,3-dione

To a stirred solution of 14-[[2-(2,6-dioxopiperidin-3-yl)-1,3-dioxo-2,3-dihydro-1H-isoindol-4-yl]amino]-3,6,9,12-tetraoxatetradecanoic acid (40 mg, 0.079 mmol) in DMA (3 mL) were added trans-4-[[2-(butylamino)-5-[5-[(piperazin-1-yl)methyl]pyridin-2-yl]pyrimidin-4-yl]amino]cyclohexan-1-ol (35 mg, 0.079 mmol), TEA (24 mg, 0.24 mmol) and HATU (36 mg, 0.095 mmol) at room temperature under nitrogen atmosphere. After stirring for an additional 2 h at room temperature, the mixture was purified by prep-HPLC with the following conditions: Column: Sunfire Prep C¹⁸ OBD Column, 10 um, 19×250 mm; Mobile Phase A: Water (plus 0.1% FA); Mobile Phase B: ACN; Flow rate: 20 mL/min; Gradient: 18% B to 26% B in 9 min; 220/254 nm; Rt: 8.5 min. The fractions containing desired product were collected, concentrated and lyophilized to afford 4-[15-[4-([6-[2-(butylamino)-4-[[trans-4-hydroxycyclohexyl]amino]pyrimidin-5-yl]pyridin-3-yl]methyl)piperazin-1-yl]-15-oxo-4,7,10,13-tetraoxa-1-azapentadecan-1-yl]-2-(2,6-dioxopiperidin-3-yl)-2,3-dihydro-1H-isoindole-1,3-dione (20 mg, 27%) as a light yellow solid. ¹H NMR (400 MHz, Chloroform-d) δ 10.61 (br s, 1H), 8.95 (br s, 1H), 8.68 (s, 1H), 8.41 (s, 1H), 8.26 (s, 1H), 7.60 (s, 2H), 7.52 (t, J=7.9 Hz, 1H), 7.12 (d, J=7.1 Hz, 1H), 6.96 (d, J=8.6 Hz, 1H), 6.51 (t, J=5.7 Hz, 1H), 4.93 (dd, J=12.1, 5.3 Hz, 1H), 4.23 (s, 2H), 4.17-4.03 (m, 1H), 3.87-3.40 (m, 26H), 2.95-2.70 (m, 3H), 2.45 (s, 4H), 2.26-2.19 (m, 2H), 2.19-2.04 (m, 3H), 1.68 (q, J=7.4 Hz, 2H), 1.60-1.28 (m, 6H), 0.98 (t, J=7.4 Hz, 3H). LC/MS (ESI, m/z): [(M+1)]⁺=929.45

### EXAMPLE 14: 3-[4-[1-(4-[4-[2-(Butylamino)-7-[trans-4-hydroxycyclohexyl]-7H-pyrrolo[2,3-d]pyrimidin-5-yl]benzenesulfonyl]piperazin-1-yl)-3,6,9,12-tetraoxapentadecan-15-yl]-3-methyl-2-oxo-2,3-dihydro-1H-1,3-benzodiazol-1-yl]piperidine-2,6-dione formate (I-13)

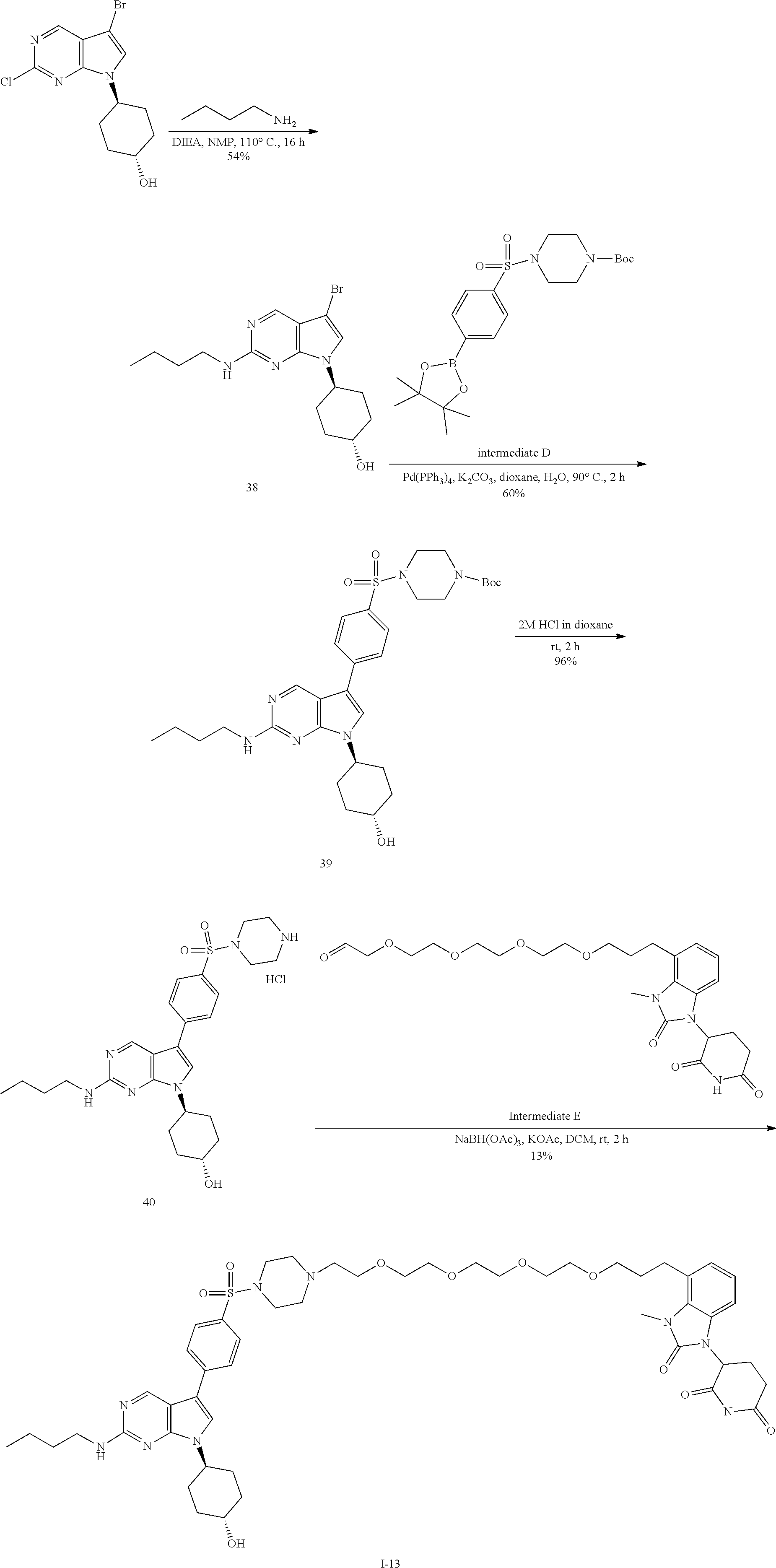

### Step 1: trans-4-[5-Bromo-2-(butylamino)-7H-pyrrolo[2,3-d]pyrimidin-7-yl]cyclohexan-1-ol

To a stirred solution of trans-4-[5-bromo-2-chloro-7H-pyrrolo[2,3-d]pyrimidin-7-yl]cyclohexan-1-ol (1 g, 3.03 mmol) in NMP (20 mL) were added DIEA (0.98 g, 7.56 mmol) and butan-1-amine (0.66 g, 9.07 mmol) at room temperature. The resulting solution was stirred at 110° C. for 16 h. After cooling down to room temperature, the mixture was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 20 min; Flow rate: 80 mL/min; Detector: 220/254 nm; desired fractions were collected at 51% B and concentrated under reduced pressure to afford trans-4-[5-bromo-2-(butylamino)-7H-pyrrolo[2,3-d]pyrimidin-7-yl]cyclohexan-1-ol (600 mg, 54%) as an orange solid. ¹H NMR (400 MHz, DMSO-d₆) δ 8.35 (s, 1H), 7.36 (s, 1H), 7.00 (t, J=5.8 Hz, 1H), 4.68 (d, J=4.5 Hz, 1H), 4.38 (td, J=11.3, 5.6 Hz, 1H), 3.52 (dd, J=10.7, 4.3 Hz, 1H), 3.35-3.25 (m, 2H), 1.98-1.79 (m, 6H), 1.60-1.48 (m, 2H), 1.42-1.25 (m, 4H), 0.91 (t, J=7.3 Hz, 3H). LC/MS (ESI, m/z): [(M+1)]⁺=367.10, 369.10

### Step 2: tert-Butyl 4-[4-[2-(butylamino)-7-[trans-4-hydroxycyclohexyl]-7H-pyrrolo[2,3-d]pyrimidin-5-yl]benzenesulfonyl]piperazine-1-carboxylate

To a stirred solution of trans-4-[5-bromo-2-(butylamino)-7H-pyrrolo[2,3-d]pyrimidin-7-yl]cyclohexan-1-ol (500 mg, 1.36 mmol) in dioxane (5 mL) were added H₂O (2 mL), tert-butyl 4-[4-(4,4,5,5-tetramethyl-1,3 ,2-dioxorolan-2-yl)benzenesulfonyl]piperazine-1-carboxylate (924 mg, 2.04 mmol), K₂CO₃ (282 mg, 2.04 mmol) and Pd(PPh₃)₄ (79 mg, 0.068 mmol) at room temperature under nitrogen atmosphere. The resulting mixture was purged with nitrogen for 3 times and was stirred at 90° C. for 2 h. After cooling down to room temperature, the resulting mixture was diluted with water (20 mL) and extracted with EtOAc (3×10 mL). The combined organic layers was washed with brine (20 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 20 min; Flow rate: 80 mL/min; Detector: 220/254 nm; desired fractions were collected at 49% B and concentrated under reduced pressure to afford tert-butyl 4-[4-[2-(butylamino)-7-[trans-4-hydroxycyclohexyl]-7H-pyrrolo[2,3-d]pyrimidin-5-yl]benzenesulfonyl]piperazine-1-carboxylate (500 mg, 60%) as an orange solid. ¹H NMR (400 MHz, DMSO-d₆) δ 8.94 (s, 1H), 7.96 (m, 4H), 7.71 (d, J=8.0 Hz, 1H), 7.70-7.60 (m, 1H), 4.71 (d, J=4.5 Hz, 1H), 4.50-4.40 (m, 1H), 3.60-3.50 (m,1H), 3.41 (t, J=5.0 Hz, 4H), 3.38-3.28 (m, 2H), 2.90 (s, 4H), 2.05-1.84 (m, 6H), 1.56 (q, J=7.2 Hz, 2H), 1.43-1.34 (m, 4H), 1.34 (s, 9H), 0.93 (t, J=7.3 Hz, 3H). LC/MS (ESI, m/z): [(M+1)]⁺=613.45

### Step 3: trans-4-[2-(Butylamino)-5-[4-(piperazine-1-sulfonyl)phenylyl]-7H-pyrrolo[2,3-d]pyrimidin-7-yl]cyclohexan-1-ol

To a stirred solution of tert-butyl 4-[4-[2-(butylamino)-7-[trans-4-hydroxycyclohexyl]-7H-pyrrolo[2,3-d]pyrimidin-5-yl]benzenesulfonyl]piperazine-1-carboxylate (500 mg, 0.82 mmol) in dioxane (5 mL) was added a solution of hydrochloride in 1,4-dioxane (4 M, 5 mL) at room temperature. The reaction was stirred at room temperature for 2 h. The resulting mixture was concentrated under reduced pressure to afford trans-4-[2-(butylamino)-5-[4-(piperazine-1-sulfonyl)phenyl]-7H-pyrrolo[2,3-d]pyrimidin-7-yl]cyclohexan-1-ol (400 mg, 96%) as a yellow solid. ¹H NMR (400 MHz, DMSO-d₆) δ 9.36 (br s, 2H), 9.14 (br s, 1H), 8.33 (s, 1H), 8.18 (s, 1H), 8.04 (d, J=8.4 Hz, 2H), 7.81 (d, J=8.4 Hz, 2H), 7.70-7.55 (m, 1H), 4.52-4.45 (m, 1H), 3.63-3.52 (m, 1H), 3.47 (s, 2H), 3.21 (s, 8H), 2.09-1.88 (m, 6H), 1.60 (dt, J=8.9, 7.0 Hz, 2H), 1.48-1.32 (m, 4H), 0.96 (t, J=7.3 Hz, 3H). LC/MS (ESI, m/z): [(M+1)]⁺=513.35

### Step 4: 3-[4-[1-(4-[4-[2-(Butylamino)-7-[trans-4-hydroxycyclohexyl]-7H-pyrrolo[2,3-d]pyrimidin-5-yl]benzenesulfonyl]piperazin-1-yl)-3,6,9,12-tetraoxapentadecan-15-yl]-3-methyl-2-oxo-2,3-dihydro-1H-1,3-benzodiazol-1-yl]piperidine-2,6-dione formate

To a stirred solution of trans-4-[2-(butylamino)-5-[4-(piperazine-1-sulfonyl)phenyl]-7H-pyrrolo[2,3-d]pyrimidin-7-yl]cyclohexan-1-ol (50 mg, 0.098 mmol) in DCM (5 mL) were added 15-[1-(2,6-dioxopiperidin-3-yl)-3-methyl-2-oxo-2,3-dihydro-1H-1,3-benzodiazol-4-yl]-3,6,9,12-tetraoxapentadecanal (48 mg, 0.098 mmol), KOAc (39 mg, 0.39 mmol) and NaBH(OAc)₃ (62 mg, 0.29 mmol) at room temperature. The resulting mixture was stirred for an additional 16 h at room temperature. The resulting mixture was concentrated under reduced pressure and the residue was purified by reverse phase flash chromatography with the following conditions: Column: WelFlash™ C18-I, 20-40 μm, 40 g; Eluent A: Water (plus 10 mmol/L HCOOH); Eluent B: ACN; Gradient: 45%-65% B in 20 min; Flow rate: 80 mL/min; Detector: 220/254 nm; desired fractions were collected at 54% B and concentrated under reduced pressure to afford 3-[4-[1-(4-[4-[2-(butylamino)-7-[trans-4-hydroxycyclohexyl]-7H-pyrrolo[2,3-d]pyrimidin-5-yl]benzenesulfonyl]piperazin-1-yl)-3,6,9,12-tetraoxapentadecan-15-yl]-3-methyl-2-oxo-2,3-dihydro-1H-1,3-benzodiazol-1-yl]piperidine-2,6-dione formate (13 mg, 13%) as a yellow solid. ¹H NMR (400 MHz, Methanol-d₄) δ 8.80 (s, 1H), 8.29 (s, 1H), 7.88 (d, J=8.4 Hz, 2H), 7.80 (d, J=8.4 Hz, 2H), 7.65 (s, 1H), 7.06-6.90 (m, 3H), 5.33 (dd, J=12.1, 5.3 Hz, 1H), 4.55 (d, J=12.2 Hz, 1H), 3.77-3.48 (m, 17H), 3.51-3.42 (m, 4H), 3.13 (s, 4H), 3.01 (dd, J=8.7, 6.9 Hz, 2H), 2.96-2.75 (m, 9H), 2.19-2.10 (m, 3H), 2.09-2.02 (m, 4H), 1.86 (dq, J=12.0, 6.0 Hz, 2H), 1.73-1.61 (m, 2H), 1.60-1.40 (m, 4H), 1.02 (t, J=7.4 Hz, 3H). LC/MS (ESI, m/z): [(M+1)]⁺=988.65

### EXAMPLE 15: 3-(5-(1-(4-(4-(2-(Butylamino)-7-(trans-4-hydroxycyclohexyl)-7H-pyrrolo[2,3-d]pyrimidin-5-yl)phenylsulfonyl)piperazin-1-yl)-3,6,9,12-tetraoxapentadecan-15-yl)-3-methyl-2-oxo-2,3-dihydrobenzo[d]imidazol-1-yl)piperidine-2,6-dione (I-12)

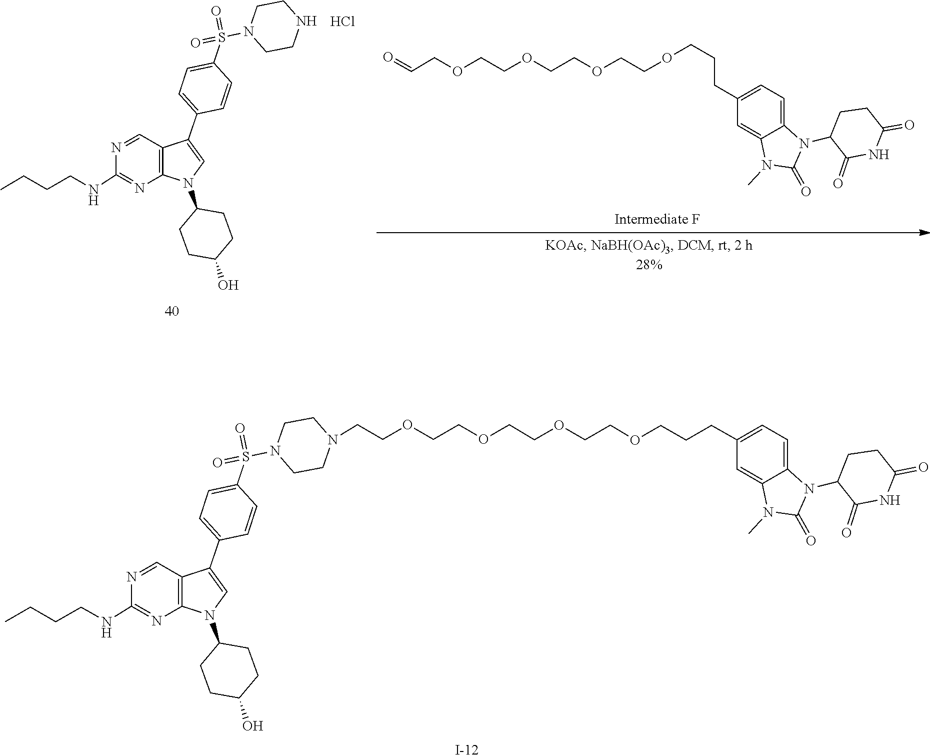

To a stirred solution of trans-4-[2-(butylamino)-5-[4-(piperazine-1-sulfonyl)phenyl]-7H-pyrrolo[2,3-d]pyrimidin-7-yl]cyclohexan-1-ol hydrochloride (50 mg, 0.091 mmol) in DCM (5 mL) were added 15-[1-(2,6-dioxopiperidin-3-yl)-3-methyl-2-oxo-2,3-dihydro-1H-1,3-benzodiazol-5-yl]-3,6,9,12-tetraoxapentadecanal (45 mg, 0.091 mmol), KOAc (36 mg, 0.36 mmol) and NaBH(OAc)₃ (58 mg, 0.27 mmol) at room temperature under nitrogen atmosphere. The resulting mixture was stirred for 2 h at room temperature. The mixture was concentrated under reduced pressure. The residue was purified by prep-HPLC with the following conditions: (Column: XBridge Shield RP¹⁸ OBD Column, 5 um, 19*150 mm; Mobile Phase A: Water (plus 10 mM NH₄HCO₃); Mobile Phase B: ACN; Flow rate: 20 mL/min; Gradient: 5% B to 39% B in 10 min; 254/220 nm; Rt: 7.82 min). Fractions containing desired product were combined and concentrated under reduced pressure to afford 3-(5-(1-(4-(4-(2-(Butylamino)-7-(trans-4-hydroxycyclohexyl)-7H-pyrrolo[2,3-d]pyrimidin-5-yl)phenylsulfonyl)piperazin-1-yl)-3,6,9,12-tetraoxapentadecan-15-yl)-3-methyl-2-oxo-2,3-dihydrobenzo[d]imidazol-1-yl)piperidine-2,6-dione (25 mg, 28%) as a white solid. ¹H NMR (400 MHz, DMSO-d₆) δ 11.11 (s, 1H), 8.94 (s, 1H), 7.95 (d, J=8.0 Hz, 2H), 7.89 (s, 1H), 7.70 (d, J=8.0 Hz, 2H), 7.05-6.96 (m, 3H), 6.85 (d, J=8.0 Hz, 1H), 5.34 (dd, J=12.9, 5.3 Hz, 1H), 4.73 (d, J=4.4 Hz, 1H), 4.48-4.38 (m, 1H), 3.60-3.50 (m, 1H), 3.51-3.38 (m, 15H), 3.33-3.29 (m, 4H), 2.95-2.83 (m, 5H), 2.70-2.58 (m, 4H), 2.50-2.40 (m, 7H), 2.00-1.94 (m, 7H), 1.83-1.75 (m, 2H), 1.60-1.50 (m, 2H), 1.40-1.29 (m, 4H), 0.93 (t, J=7.3 Hz, 3H). LC/MS (ESI, m/z): [(M/2 +1)]⁺=495.00

### KEY INTERMEDIATE PREPARATION

The following intermediates, A and B, were prepared according to the methods below and further utilized in the syntheses of compounds depicted in Examples 16 through 21.

### INTERMEDIATE A: 3-(4-Amino-3-methyl-2-oxo-2,3-dihydro-1H-1,3-benzodiazol-1-yl)piperidine-2,6-dione

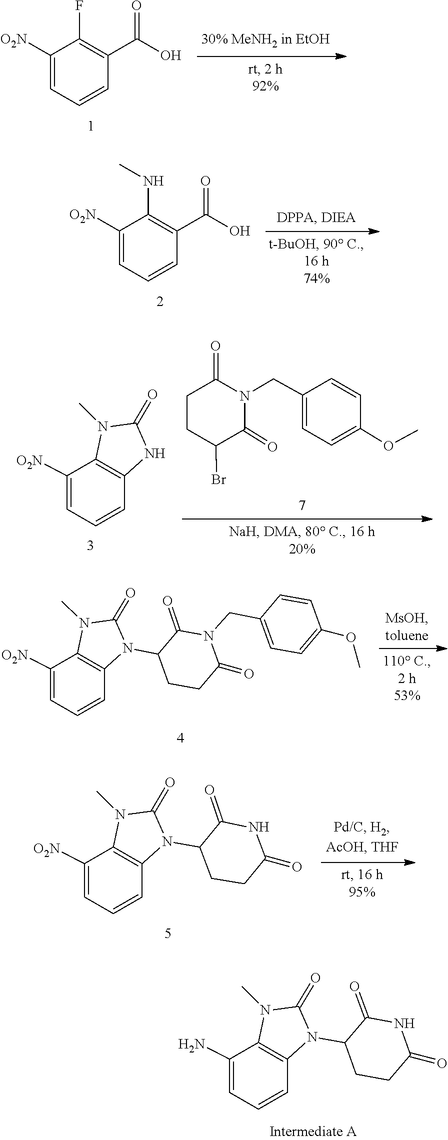

### Step 1: 2-(Methylamino)-3-nitrobenzoic acid

To a stirred solution of methanamine (30% in EtOH, w/w, 500 mL) was added 2-fluoro-3-nitrobenzoic acid (23.5 g, 0.13 mol) in portions at 0° C. The resulting mixture was stirred for 2 h at room temperature under nitrogen atmosphere and then concentrated under reduced pressure. The residue was dissolved in water (200 mL) and the resulting solution was acidified to pH=5˜6 by addition of 1 N hydrochloric acid at 0° C. The precipitated solids were collected by filtration and dried in a vacuum oven to afford 2-(methylamino)-3-nitrobenzoic acid (23 g, 92%) as a yellow solid. ¹H NMR (400 MHz, CDCl₃) δ 8.21 (d, J=7.8 Hz, 1H), 8.04 (d, J=8.1 Hz, 1H), 6.77 (t, J=8.0 Hz, 1H), 2.86 (s, 3H). LC/MS (ESI, m/z): [(M+1)]⁺=197.2

### Step 2: 1-Methyl-7-nitro-2,3-dihydro-1H-1,3-benzodiazol-2-one

To a stirred suspension of 2-(methylamino)-3-nitrobenzoic acid (13 g, 66.3 mmol) in t-BuOH (300 mL) were added N-ethyldiisopropylamine (25.7 g, 198.8 mmol) and diphenylphosphoryl azide (17.2 mL, 62.5 mmol) dropwise at 0° C. under nitrogen atmosphere. The resulting mixture was stirred for 16 h at 90° C. The resulting mixture was cooled down to room temperature and filtered. The filter cake was triturated with MeOH (200 mL) and filtered. The filter cake was dried in a vacuum oven to afford 1-methyl-7-nitro-2,3-dihydro-1H-1,3-benzodiazol-2-one (9.5 g, 74%) as a yellow solid. ¹H NMR (400 MHz, DMSO-d₆) δ 11.64 (s, 1H), 7.61 (dd, J=8.4, 1.1 Hz, 1H), 7.33 (dd, J=7.8, 1.1 Hz, 1H), 7.15 (t, J=8.1 Hz, 1H), 3.36 (s, 3H). LC/MS (ESI, m/z): [(M−1)]⁻=192.1

### Step 3: 1-[(4-Methoxyphenyl)methy]-3-(3-methyl-4-nitro-2-oxo-2,3-dihydro-1H-1,3-benzodiazol-1-yl)piperidine-2,6-dione

A solution of 1-methyl-7-nitro-2,3-dihydro-1H-1,3-benzodiazol-2-one (10.46 g, 54.14 mmol) in DMA (250 mL) was treated with NaH (60% in mineral oil, 2.18 g, 90.78 mmol) for 1 h at 0° C. under nitrogen atmosphere followed by the addition of 3-bromo-1-[(4-methoxyphenyl)methyl]piperidine-2,6-dione (13 g, 41.64 mmol) at 0° C. The resulting mixture was stirred for 16 h at 80° C. The resulting mixture was cooled down to room temperature, acidified to pH=5 by addition of AcOH and diluted with water (500 mL). The resulting mixture was extracted with ethyl acetate (3×400 mL). The combined organic layers was washed with brine (600 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L formic acid); Eluent B: ACN; Gradient: 45%-70% B in 20 min; Flow rate: 100 mL/min; Detector: 220/254 nm; desired fractions were collected at 65% B and concentrated under reduced pressure to afford 1-[(4-methoxyphenyl)methyl]-3-(3-methyl-4-nitro-2-oxo-2,3-dihydro-1H-1,3-benzodiazol-1-yl)piperidine-2,6-dione (3.6 g, 20%) as a yellow solid. ¹H NMR (400 MHz, DMSO-d₆) δ 7.69 (dd, J=8.4, 1.0 Hz, 1H), 7.47 (d, J=7.8 Hz, 1H), 7.25-7.16 (m, 3H), 6.90-6.82 (m, 2H), 5.68 (dd, J=12.9, 5.4 Hz, 1H), 4.83 (d, J=14.3 Hz, 1H), 4.76 (d, J=14.3 Hz, 1H), 3.72 (s, 3H), 3.41 (s, 3H), 3.13-2.99 (m, 1H), 2.90-2.70 (m, 2H), 2.17-2.05 (m, 1H). LC/MS (ESI, m/z): [(M+1)]⁺=425.25

### Step 4: 3-(3-Methyl-4-nitro-2-oxo-2,3-dihydro-1H-1,3-benzodiazol-1-yl)piperidine-2,6-dione

To a suspension of 1-[(4-methoxyphenyl)methyl]-3-(3-methyl-4-nitro-2-oxo-2,3-dihydro-1H-1,3-benzodiazol-1-yl)piperidine-2,6-dione (3.7 g, 8.7 mmol) in toluene (20 mL) was added methanesulfonic acid (20 mL). The resulting mixture was stirred for 2 h at 110° C. After cooling down to room temperature, the resulting mixture was added dropwise to ice water (500 mL) at 0° C. The precipitated solids were collected by filtration and washed with water (3×100 mL). The collected solids were taken up with DMF (50 mL) and purified by reverse phase flash chromatography with the following conditions: Column: WelFlash™ C18-I, 20-40 μm, 330 g; Eluent A: Water (plus 10 mmol/L formic acid); Eluent B: ACN; Gradient: 40%-60% B in 15 min; Flow rate: 100 mL/min; Detector: 220/254 nm; desired fractions were collected at 55% B and concentrated under reduced pressure to afford 3-(3-methyl-4-nitro-2-oxo-2,3-dihydro-1H-1,3-benzodiazol-1-yl)piperidine-2,6-dione (1.4 g, 53%) as a yellow solid. ¹H NMR (400 MHz, DMSO-d₆) δ 11.19 (s, 1H), 7.69 (d, J=8.5 Hz, 1H), 7.56 (d, J=7.9 Hz, 1H), 7.24 (t, J=8.2 Hz, 1H), 5.52 (dd, J=12.7, 5.3 Hz, 1H), 3.42 (s, 3H), 3.01-2.84 (m, 1H), 2.82-2.60 (m, 2H), 2.10 (d, J=11.0 Hz, 1H). LC/MS (ESI, m/z): [(M−1)]⁻=303.10

### Step 5: 3-(4-Amino-3-methyl-2-oxo-2,3-dihydro-1H-1,3-benzodiazol-1-yl)piperidine-2,6-dione

To a solution of 3-(3-methyl-4-nitro-2-oxo-2,3-dihydro-1H-1,3-benzodiazol-1-yl)piperidine-2,6-dione (1.4 g, 4.6 mmol) in AcOH (200 mL) and THF (100 mL) was added palladium on charcoal (685 mg, 10% w/w) under nitrogen atmosphere. The mixture was purged with hydrogen three times and then hydrogenated at room temperature for 16 h. The mixture was filtered through a Celite pad and concentrated under reduced pressure to afford 3-(4-amino-3-methyl-2-oxo-2,3-dihydro-1H-1,3-benzodiazol-1-yl)piperidine-2,6-dione (1.2 g, 95%) as a light pink solid. ¹H NMR (400 MHz, DMSO-d₆) δ 6.75 (t, J=7.9 Hz, 1H), 6.42 (m, 2H), 5.26 (dd, J=13.2, 5.3 Hz, 1H), 4.97 (br s, 2H), 3.58 (s, 3H), 2.88 (t, J=14.5 Hz, 1H), 2.75-2.56 (m, 2H), 1.97 (d, J=12.0 Hz, 1H). LC/MS (ESI, m/z): [(M+1)]⁺=275.05

### Intermediate B: [[1-(2,6-Dioxopiperidin-3-yl)-3-methyl-2-oxo-2,3-dihydro-1H-1,3-benzodiazol-4-yl]amino]-3,6,9,12-tetraoxatetradecanal

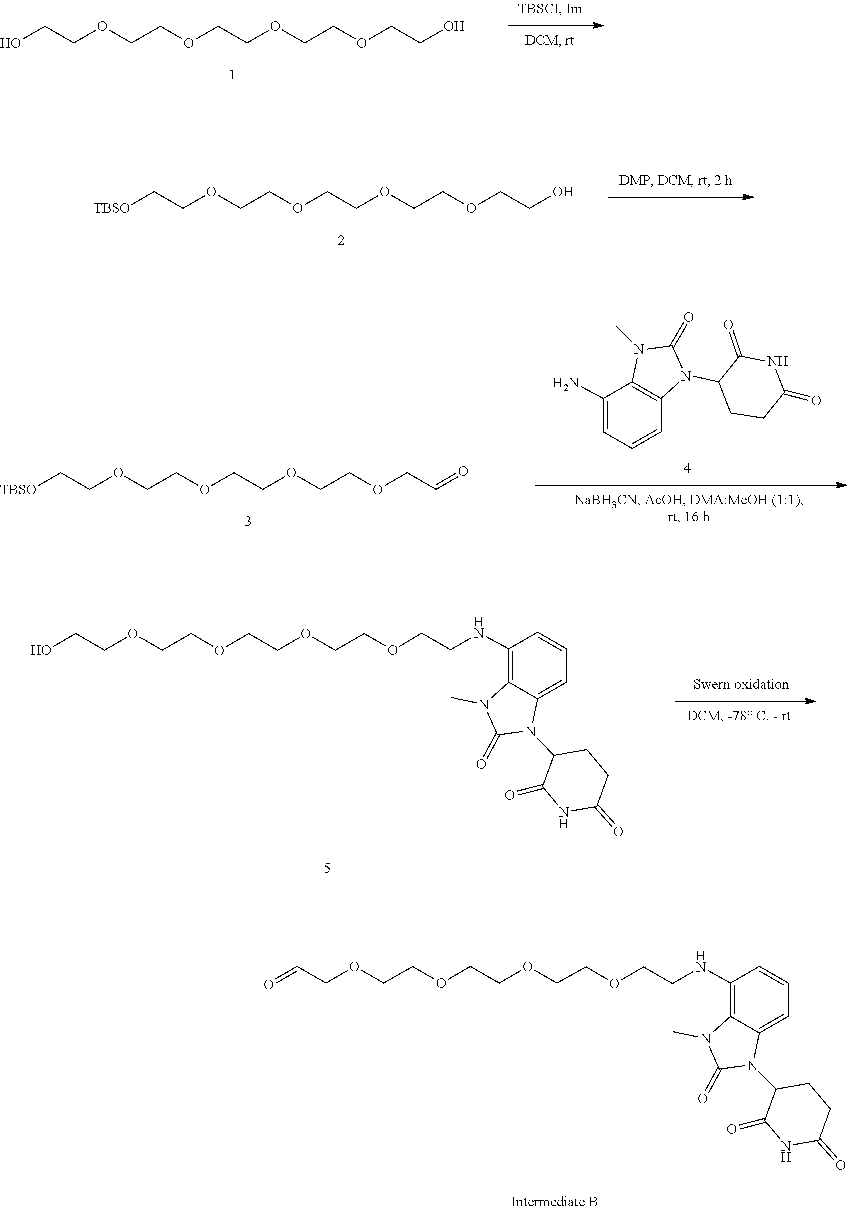 [SMILES: OCCOCCOCCOCCOCCO ; CC(C)(C)[Si](C)(C)OCCOCCOCCOCCOCCO ; Cn1c(=O)n(C2CCC(=O)NC2=O)c2cccc(N)c21 ; CC(C)(C)[Si](C)(C)OCCOCCOCCOCCOCC=O ; Cn1c(=O)n(C2CCC(=O)NC2=O)c2cccc(NCCOCCOCCOCCOCCO)c21 ; Cn1c(=O)n(C2CCC(=O)NC2=O)c2cccc(NCCOCCOCCOCCOCC=O)c21]

### Step 1: 2,2,3,3-Tetramethyl-4,7,10,13,16-pentaoxa-3-silaoctadecan-18-ol

To a stirred solution of 3,6,9,12-tetraoxatetradecane-1,14-diol (40 g, 167.87 mmol) in DCM (300 mL) were added imidazole (13.7 g, 201.44 mmol) and tert-butyl(chloro)dimethylsilane (25.3 g, 167.87 mmol) at 0° C. under nitrogen atmosphere. The resulting solution was stirred for 16 h at room temperature. The reaction was quenched with water (400 mL) and extracted with EtOAc (2×300 mL). The combined organic layers was washed with brine (200 mL) and dried over anhydrous Na₂SO₄. After filtration, the filtrate was concentrated under reduced pressure. The residue was purified by silica gel column chromatography, eluted with EtOAc to afford 2,2,3,3-tetramethyl-4,7,10,13,16-pentaoxa-3-silaoctadecan-18-ol (20 g, 34%) as a light yellow oil. ¹H NMR (400 MHz, CDCl₃) δ 3.77-3.48 (m, 20H), 2.82-2.69 (m, 1H), 0.86 (d, J=3.0 Hz, 9H), 0.04 (d, J=3.0 Hz, 6H). LC/MS (ESI, m/z): [(M+1)]⁺=353.4

### Step 2: 2,2,3,3-Tetramethyl-4,7,10,13,16-pentaoxa-3-silaoctadecan-18-al

To a stirred solution of 2,2,3,3-tetramethyl-4,7,10,13,16-pentaoxa-3-silaoctadecan-18-ol (572 mg, 1.62 mmol) in DCM (5 mL) was added Dess-Martin reagent (1.03 g, 2.43 mmol) under nitrogen atmosphere. The resulting mixture was stirred for 2 h at room temperature. The reaction was quenched by a saturated aqueous solution of NaHCO₃ (50 mL) and the resulting mixture was extracted with DCM (3×30 mL). The combined organic layers was washed with brine (50 mL) and dried over anhydrous Na₂SO₄. After filtration, the filtrate was concentrated under reduced pressure. The residue was purified by silica gel column chromatography, eluted with 5%-15% ethyl acetate in petroleum ether to afford 2,2,3,3-tetramethyl-4,7,10,13,16-pentaoxa-3-silaoctadecan-18-al (300 mg, 53%) as a light yellow oil. ¹H NMR (400 MHz, CDCl₃) δ 9.74 (d, J=0.9 Hz, 1H), 4.17 (d, J=0.9 Hz, 2H), 3.81-3.69 (m, 6H), 3.69-3.62 (m, 8H), 3.57 (t, J=5.5 Hz, 2H), 0.90 (s, 9H), 0.07 (s, 6H). LC/MS (ESI, m/z): [(M+18)]⁺=368.2

### Step 3: 3-[4-(15-Hydroxy-4,7,10,13-tetraoxa-1-azapentadecan-1-yl)-3-methyl-2-oxo-2,3-dihydro-1H-1,3-benzodiazol-1-yl]piperidine-2,6-dione

To a solution of 3-(4-amino-3-methyl-2-oxo-2,3-dihydro-1H-1,3-benzodiazol-1-yl)piperidine-2,6-dione (1.37 g, 4.99 mmol) in MeOH (35 mL) and DMA (35 mL) were added 2,2,3,3-tetramethyl-4,7,10,13,16-pentaoxa-3-silaoctadecan-18-al (2.63 g, 7.49 mmol), AcOH (1.43 mL, 23.83 mmol) and NaBH₃CN (0.94 g, 14.98 mmol) at 0° C. under nitrogen atmosphere. The resulting mixture was stirred for 16 h at room temperature. The resulting mixture was concentrated under reduced pressure and the residue was purified by reverse phase flash chromatography with the following conditions: Column: WelFlash™ C18-I, 20-40 μm, 330 g; Eluent A: Water (plus 10 mmol/L formic acid); Eluent B: ACN; Gradient: 25%-50% B in 15 min; Flow rate: 100 mL/min; Detector: 220/254 nm; desired fractions were collected at 45% B and concentrated under reduced pressure to afford 3-[4-(15-hydroxy-4,7,10,13-tetraoxa-1-azapentadecan-1-yl)-3-methyl-2-oxo-2,3-dihydro-1H-1,3-benzodiazol-1-yl]piperidine-2,6-dione (1.5 g, 61%) as a green semi-solid. ¹H NMR (400 MHz, DMSO-d₆) δ 11.08 (s, 1H), 6.89 (t, J=8.1 Hz, 1H), 6.53 (d, J=7.9 Hz, 1H), 6.47 (d, J=8.2 Hz, 1H), 5.30 (dd, J=12.7, 5.3 Hz, 1H), 5.02 (s, 1H), 4.68 (t, J=5.8 Hz, 1H), 4.61 (s, 2H), 3.62 (s, 3H), 3.60-3.47 (m, 10H), 3.45-3.39 (m, 6H), 3.26 (q, J=6.1, 5.5 Hz, 2H), 2.95-2.83 (m, 1H), 2.77-2.57 (m, 2H), 2.03-1.95 (m, 1H). LC/MS (ESI, m/z): [(M+1)]⁺=495.35

### Step 4: 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal

To a stirred solution of oxalyl chloride (539 mg, 4.25 mmol) in DCM (30 mL) was added DMSO (442 mg, 5.66 mmol) dropwise at −78° C. under nitrogen atmosphere. The resulting solution was stirred for 30 min at −78° C. followed by the addition of 3-[4-(15-hydroxy-4,7,10,13-tetraoxa-1-azapentadecan-1-yl)-3-methyl-2-oxo-2,3-dihydro-1H-1,3-benzodiazol-1-yl]piperidine-2,6-dione (700 mg, 1.42 mmol) in DCM (20 mL) over 5 min. The resulting mixture was kept at −78° C. for another 1 h. To the above mixture was added TEA (1145 mg, 11.32 mmol). The resulting mixture was stirred for an additional 30 min at −78° C. The resulting mixture was diluted with water (60 mL) and extracted with DCM (2×50 mL). The combined organic layers was washed with brine (50 mL) and dried over anhydrous Na₂SO₄. After filtration, the filtrate was concentrated under reduced pressure to give 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (650 mg, crude) as a light yellow oil. LC/MS (ESI, m/z): [(M+1)]⁺=493.35

### SYNTHESIS OF FINAL TARGETS

### EXAMPLE 16: Trans-3-[4-[15-(4-[4-[2-(butylamino)-7-[-4-hydroxycyclohexyl]-7H-pyrrolo[2,3-d]pyrimidin-5-yl]benzenesulfonyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione (I-35)

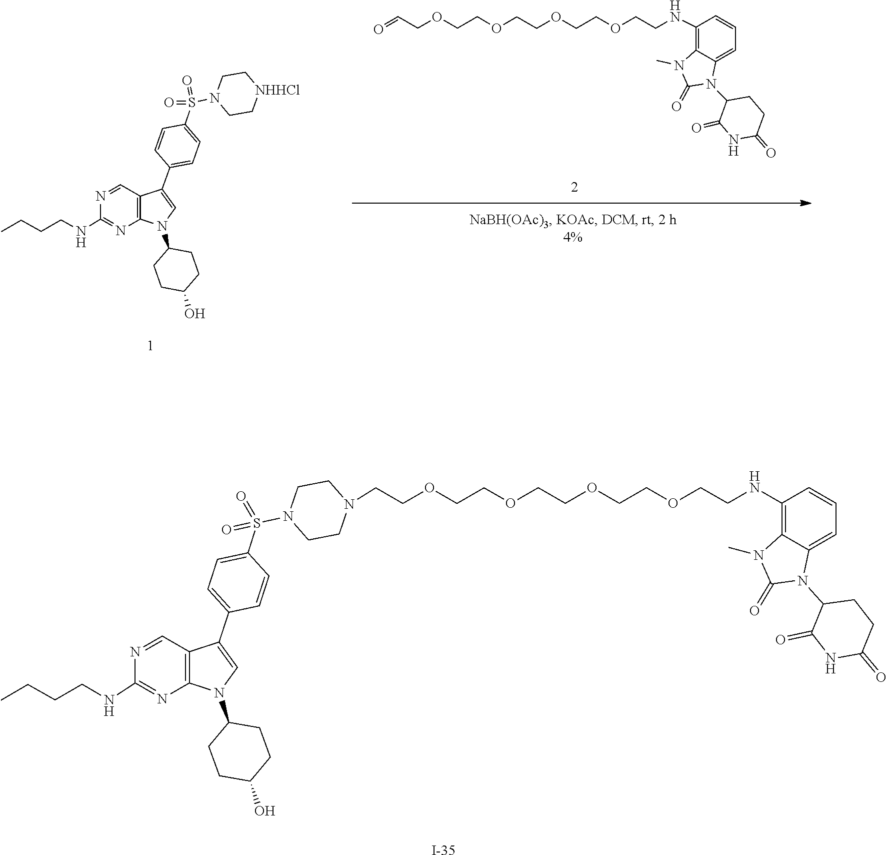

To a stirred solution of trans-4-[2-(butylamino)-5-[4-(piperazine-1-sulfonyl)phenyl]-7H-pyrrolo[2,3-d]pyrimidin-7-yl]cyclohexan-1-ol hydrochloride (145 mg, 0.26 mmol) in DCM (20 mL) were added 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (130 mg, 0.26 mmol), KOAc (104 mg, 1.06 mmol) and NaBH(OAc)₃ (168 mg, 0.79 mmol) at ambient temperature. The resulting mixture was stirred for 2 h at room temperature. Upon completion, the resulting mixture was concentrated under reduced pressure. The crude product was purified by Prep-HPLC with the following conditions: Column: XBridge Shield RP18 OBD Column, 5 um, 19×150 mm; Mobile Phase A: Water (plus 10 mmol/L NH₄HCO₃); Mobile Phase B: ACN; Flow rate: 25 mL/min; Gradient: 28% B to 44% B in 10 min; Detector: 254/220 nm; Rt: 9.82 min) to afford trans-3-[4-[15-(4-[4-[2-(butylamino)-7-[4-hydroxycyclohexyl]-7H-pyrrolo[2,3-d]pyrimidin-5-yl]benzenesulfonyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione (10 mg, 4%) as a white solid. ¹H NMR (400 MHz, CD₃OD) δ 8.81 (s, 1H), 7.92-7.85 (m, 2H), 7.79 (d, J=8.5 Hz, 2H), 7.65 (s, 1H), 6.97 (t, J=8.1 Hz, 1H), 6.56 (dd, J=12.4, 8.1 Hz, 2H), 5.27 (dd, J=12.1, 5.4 Hz, 1H), 4.60-4.51 (m, 1H), 3.80-3.69 (m, 6H), 3.66-3.61 (m, 4H), 3.59-3.39 (m, 12H), 3.30 (d, J=5.2 Hz, 2H), 3.11-2.97 (m, 4H), 2.94-2.82 (m, 1H), 2.85-2.70 (m, 2H), 2.66-2.57 (m, 4H), 2.54 (t, J=5.3 Hz, 2H), 2.19-1.96 (m, 7H), 1.77-1.63 (m, 2H), 1.61-1.41 (m, 4H), 1.02 (t, J=7.3 Hz, 3H). LC/MS (ESI, m/z): [(M+1)]⁺=989.55

### EXAMPLE 17: trans-3-(4-[15-[4-([4-[2-(butylamino)-7-[4-hydroxycyclohexyl]-7H-pyrrolo[2,3-d]pyrimidin-5-yl]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione (I-36)

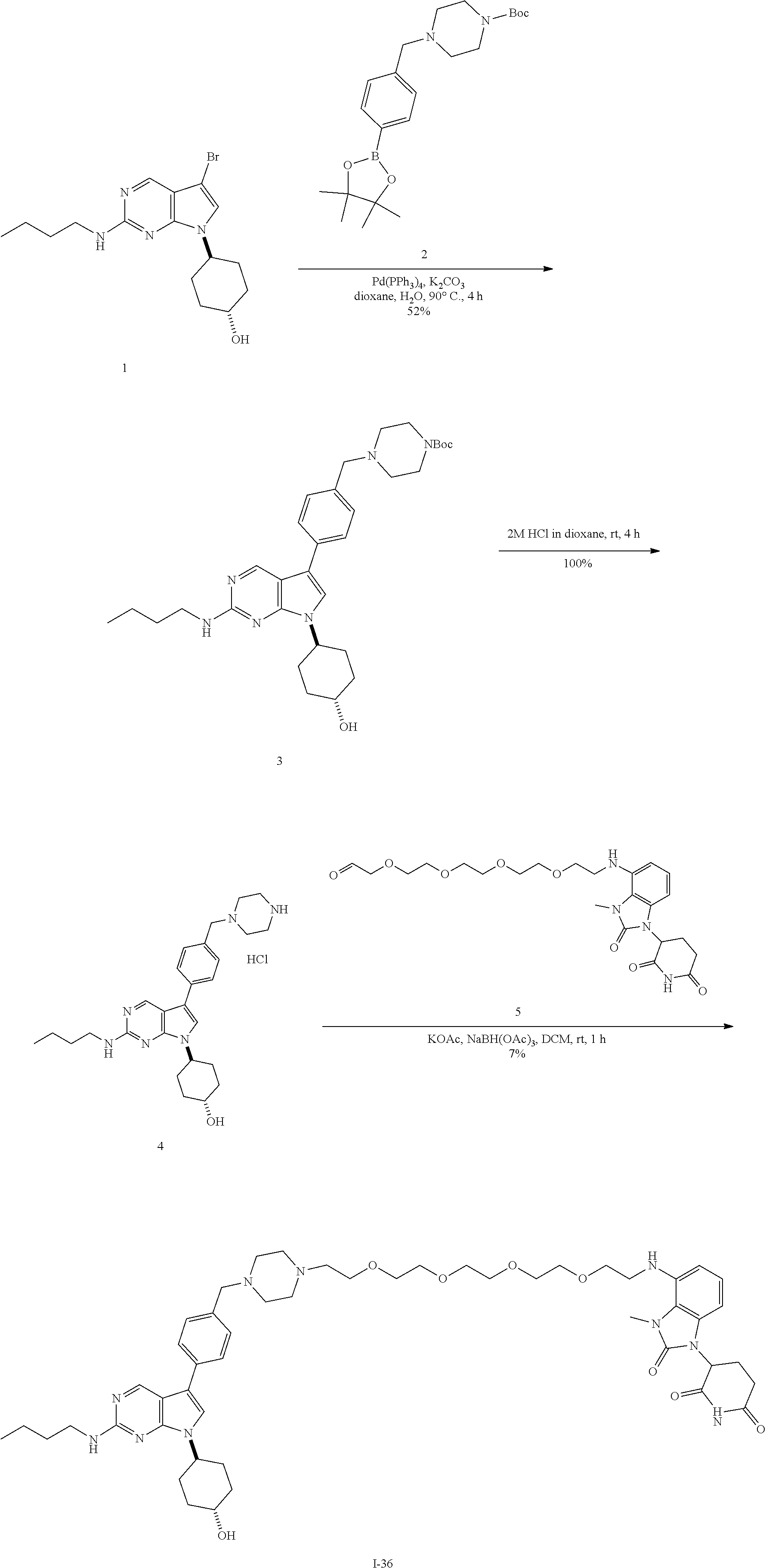

### Step 1: Tert-butyl-4-([4-[2-(butylamino)-7-[trans-4-hydroxycyclohexyl]-7H-pyrrolo[2,3-d]pyrimidin-5-yl]phenyl]methyl)piperazine-1-carboxylate

To a stirred solution of trans-4-[5-bromo-2-(butylamino)-7H-pyrrolo[2,3-d]pyrimidin-7-yl]cyclohexan-1-ol (2.5 g, 6.81 mmol) in 1,4-dioxane (45 mL) and H₂O (15 mL) were added K₂CO₃ (2.82 g, 20.42 mmol), tert-butyl 4-(4-(4,4,5,5-tetramethyl-1,3,2-dioxaborolan-2-yl)benzyl)piperazine-1-carboxylate (2.62 g, 8.17 mmol) and Pd(PPh₃)₄ (0.79 g, 0.68 mmol) at room temperature under nitrogen atmosphere. The resulting mixture was stirred at 90° C. for 4 h under nitrogen atmosphere. After cooling down to room temperature, the resulting mixture was diluted with water (100 mL). The resulting mixture was extracted with ethyl acetate (3×50 mL). The combined organic layers was dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 50%-75% B in 25 min; Flow rate: 100 mL/min; Detector: 220/254 nm; desired fractions were collected at 70% B and concentrated under reduced pressure to afford tert-butyl-4-([4-[2-(butylamino)-7-[trans-4-hydroxycyclohexyl]-7H-pyrrolo[2,3-d]pyrimidin-5-yl]phenyl]methyl)piperazine-1-carboxylate (2.0 g, 52%) as an off white solid. ¹H NMR (400 MHz, DMSO-d₆) δ 8.82 (s, 1H), 7.63 (d, J=7.9 Hz, 2H), 7.58 (s, 1H), 7.32 (d, J=7.9 Hz, 2H), 6.84 (t, J=5.8 Hz, 1H), 4.71 (d, J=4.4 Hz, 1H), 4.47-4.37 (m, 1H), 3.57-3.49 (m, 1H), 3.48 (s, 2H), 3.33-3.28 (m, 6H), 2.33 (t, J=5.0 Hz, 4H), 2.03-1.83 (m, 6H), 1.56 (p, J=7.2 Hz, 2H), 1.45-1.30 (m, 13H), 0.93 (t, J=7.3 Hz, 3H). LC/MS (ESI, m/z): [(M+1)]⁺=563.35

### Step 2: trans-4-[2-(butylamino)-5-[4-[(piperazin-1-yl)methyl]phenyl]-7H-pyrrolo[2,3-d]pyrimidin-7-yl]cyclohexan-1-ol hydrochloride

To a stirred solution of tert-butyl 4-([4-[2-(butylamino)-7-[trans-4-hydroxycyclohexyl]-7H-pyrrolo[2,3-d]pyrimidin-5-yl]phenyl]methyl)piperazine-1-carboxylate (2.0 g, 3.55 mmol) in DCM (30 mL) was added a solution of hydrochloride in 1,4-dioxane (4 M, 20 mL) at room temperature. After stirring for 2 h at room temperature, the resulting solution was concentrated under reduced pressure to give trans-4-[2-(butylamino)-5-[4-[(piperazin-1-yl)methyl]phenyl]-7H-pyrrolo[2,3-d]pyrimidin-7-yl]cyclohexan-1-ol hydrochloride (2.0 g, crude) as a light yellow solid. ¹H NMR (400 MHz, DMSO-d₆) δ 9.63 (br s, 2H), 9.07 (s, 1H), 8.15 (s, 1H), 7.82 (d, J=7.9 Hz, 2H), 7.72 (s, 2H), 4.52-4.41 (m, 2H), 3.57 (s, 6H), 3.47 (s, 7H), 2.08-1.83 (m, 6H), 1.61 (p, J=7.1 Hz, 2H), 1.39 (m, 4H), 0.96 (t, J=7.3 Hz, 3H). LC/MS (ESI, m/z): [(M+1)]⁺=463.25

### Step 3: trans-3-(4-[15-[4-([4-[2-(butylamino)-7-[4-hydroxycyclohexyl]-7H-pyrrolo[2,3-d]pyrimidin-5-y]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione

To a stirred solution of trans-4-[2-(butylamino)-5-[4-[(piperazin-1-yl)methyl]phenyl]-7H-pyrrolo[2,3-d]pyrimidin-7-yl]cyclohexan-1-ol hydrochloride (150 mg, 0.30 mmol) in DCM (10 mL) were added 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (148 mg, 0.30 mmol), KOAc (118 mg, 1.20 mmol) and NaBH(OAc)₃ (191 mg, 0.90 mmol) at ambient temperature under nitrogen atmosphere. After stirring for an additional 1 h, the resulting mixture was concentrated under reduced pressure and was purified by reverse phase flash chromatography with the following conditions: Column: WelFlash™ C18-I, 20-40 μm, 80 g; Eluent A: Water (plus 10 mmol/L NH₄HCO₃); Eluent B: ACN; Gradient: 25%-55% B in 25 min; Flow rate: 60 mL/min; Detector: 220/254 nm; desired fractions were collected at 41% B and concentrated under reduced pressure to afford the product, which was further purified by Prep-HPLC with the following conditions: Column: XBridge Shield RP18 OBD Column, 5 um, 19×150 mm; Mobile Phase A: Water (plus 10 mmol/L FA); Mobile Phase B: ACN; Flow rate: 25 mL/min; Gradient: 10% B to 30% B in 7 min; 254 nm; Rt: 7 min to afford trans-3-(4-[15-[4-([4-[2-(butylamino)-7-[4-hydroxycyclohexyl]-7H-pyrrolo[2,3-d]pyrimidin-5-yl]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione (20 mg, 7%) as a light yellow solid. ¹H NMR (400 MHz, Methanol-d₄) δ 8.71 (s, 1H), 7.61 (m, 2H), 7.42-7.37 (m, 3H), 7.02-6.90 (m, 1H), 6.61-6.48 (m, 2H), 5.27 (dd, J=12.0, 5.3 Hz, 1H), 4.59-4.51 (m, 1H), 3.79-3.66 (m, 6H), 3.64-3.57 (m, 15H), 3.46 (t, J=7.1 Hz, 2H), 3.29 (t, J=5.2 Hz, 2H), 2.94-2.75 (m, 4H), 2.74-2.49 (m, 10H), 2.19-2.09 (m, 3H), 2.09-2.96 (m, 4H), 1.67 (m, 2H), 1.57-1.44 (m, 4H), 1.02 (t, J=7.4 Hz, 3H). LC/MS (ESI, m/z): [(M+1)]⁺=939.55

### EXAMPLE 18: trans-3-(4-[15-[4-([4-[6-(butylamino)-1-[4-hydroxycyclohexyl]-1H-pyrazolo[3,4-d]pyrimidin-3-yl]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione (I-37)

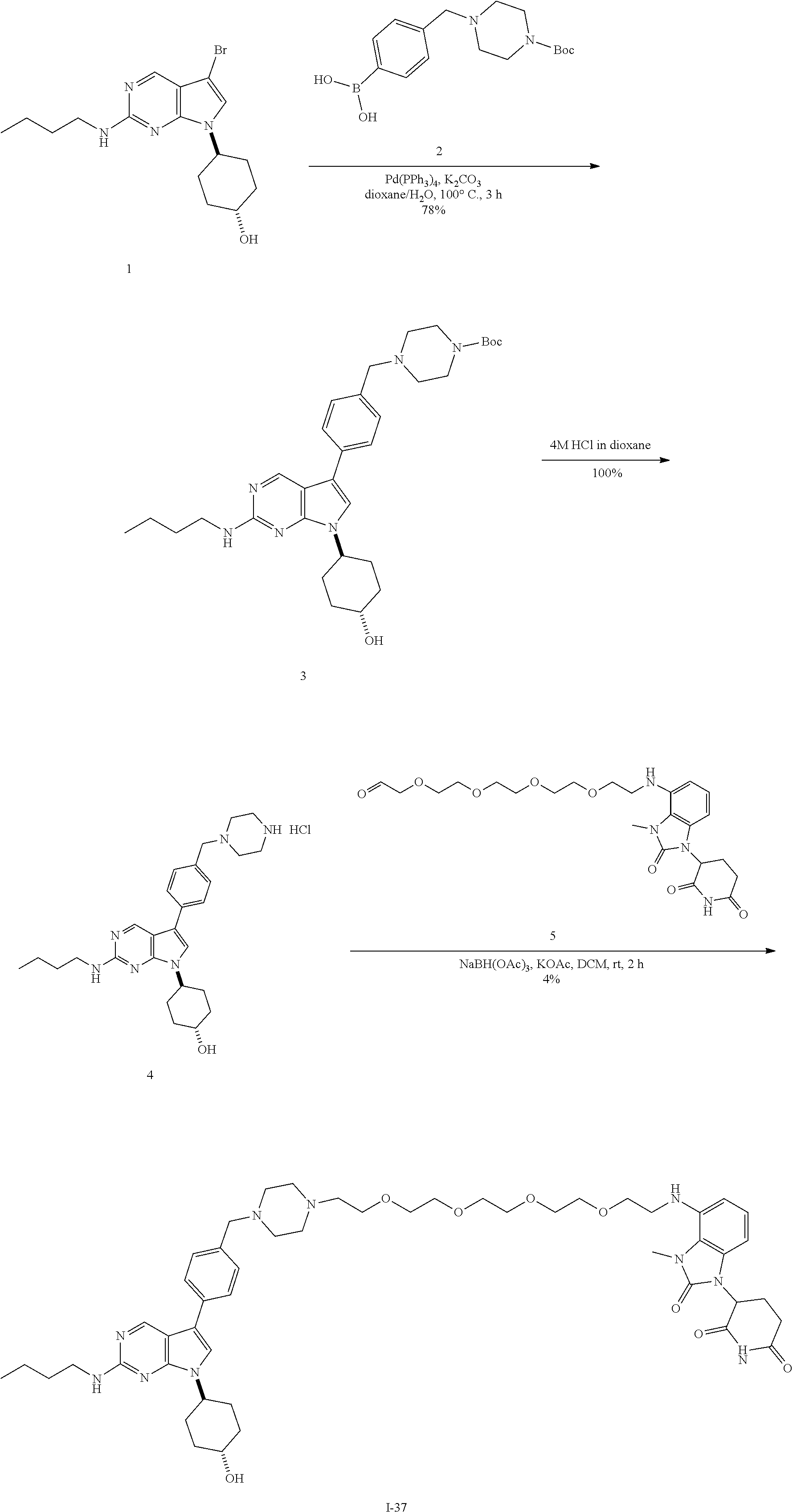

### Step 1: trans-tert-butyl 4-([4-[2-(butylamino)-7-[4-hydroxycyclohexyl]-7H-pyrrolo[2,3-d]pyrimidin-5-yl]phenyl]methyl)piperazine-1-carboxylate

To a stirred solution of trans-4-[5-bromo-2-(butylamino)-7H-pyrrolo[2,3-d]pyrimidin-7-yl]cyclohexan-1-ol (1 g, 2.723 mmol) in 1,4-dioxane (30 mL) and H₂O (10 mL) were added [4-([4-[(tert-butoxy)carbonyl]piperazin-1-yl]methyl)phenyl]boronic acid (1.05 g, 3.27 mmol), K₂CO₃ (1.13 g, 8.17 mmol) and Pd(PPh₃)₄ (0.31 g, 0.27 mmol) at room temperature under nitrogen atmosphere. The resulting mixture was stirred for 3 h at 100° C. under nitrogen atmosphere. Upon completion, the resulting mixture was cooled to room temperature, diluted with water (20 mL) and extracted with ethyl acetate (3×50 mL). The combined organic layers was dried over Na₂SO₄ and condensed under reduced pressure. The residue was purified by reverse phase Column: WelFlash™ C18-I, 20-40 μm, 330 g; Eluent A: Water (plus 10 mmol/L NH₄HCO₃); Eluent B: ACN; Gradient: 40%-70% B in 25 min; Flow rate: 100 mL/min; Detector: 220/254 nm; desired fractions were collected at 68% B and concentrated under reduced pressure to afford trans-tert-butyl 4-([4-[2-(butylamino)-7-[4-hydroxycyclohexyl]-7H-pyrrolo[2,3-d]pyrimidin-5-yl]phenyl]methyl)piperazine-1-carboxylate (1.2 g, 78%) as a white solid. ¹H NMR (400 MHz, DMSO-d₆) δ 9.09 (s, 1H), 7.92 (d, J=7.8 Hz, 2H), 7.42 (d, J=8.0 Hz, 2H), 7.32 (d, J=7.4 Hz, 1H), 4.69 (d, J=4.3 Hz, 1H), 4.59-4.43 (m, 1H), 3.64-3.49 (m, 2H), 3.41-3.34 (m, 3H), 2.35 (t, J=5.0 Hz, 4H), 2.15-1.85 (m, 6H), 1.71-1.50 (m, 2H), 1.46-1.31 (m, 13H), 1.30-1.17 (m, 4H), 0.93 (d, J=7.2 Hz, 3H). LC/MS (ESI, m/z): [(M+1)]⁺=564.40

### Step 2: trans-(4-[6-(butylamino)-3-[4-[(piperazin-1-yl)methyl]phenyl]-1H-pyrazolo[3,4-d]pyrimidin-1-yl]cyclohexan-1-ol hydrochloride

To a 4 M solution of hydrochloride in dioxane (30 mL) was added trans-tert-butyl 4-([4-[6-(butylamino)-1-[4-hydroxycyclohexyl]-1H-pyrazolo[3,4-d]pyrimidin-3-yl]phenyl]methyl)piperazine-1-carboxylate (1.2 g, 2.13 mmol). The resulting solution was stirred for 1 h at room temperature under nitrogen atmosphere. Upon completion, the mixture was concentrated under reduced pressure to give trans-4-[6-(butylamino)-3-[4-[(piperazin-1-yl)methyl]phenyl]-1H-pyrazolo[3,4-d]pyrimidin-1-yl]cyclohexan-1-ol hydrochloride (1.2 g, 100%) as a light yellow solid. ¹H MNR (400 MHz, DMSO-d₆) δ 9.77 (br s, 2H), 9.32 (s, 1H), 8.07 (d, J=7.9 Hz, 2H), 7.81 (d, J=8.0 Hz, 2H), 7.58 (t, J=9.4 Hz, 1H), 4.62-4.40 (m, 3H), 3.70-3.64 (m, 1H), 3.57 (s, 4H), 3.55-3.45 (m, 5H), 3.44-3.38 (m, 2H), 2.11-1.92 (m, 5H), 1.68-1.51 (m, 2H), 1.49-1.31 (m, 4H), 0.95 (t, J=7.4 Hz, 3H). LC/MS (ESI, m/z): [(M+1)]⁺=464.25

### Step 3: trans-3-(4-[15-[4-([4-[6-(butylamino)-1-[4-hydroxycyclohexyl]-1H-pyrazolo[3,4-d]pyrimidin-3-y]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione

To a stirred solution of trans-4-[6-(butylamino)-3-[4-[(piperazin-1-yl)methyl]phenyl]-1H-pyrazolo[3,4-d]pyrimidin-1-yl]cyclohexan-1-ol (130 mg, 0.28 mmol) in DCM (20 mL) were added 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (138 mg, 0.28 mmol), KOAc (110 mg, 1.12 mmol) and NaBH(OAc)₃ (178 mg, 0.84 mmol) at room temperature. The resulting mixture was stirred for 2 h at room temperature. Upon completion, the resulting mixture was concentrated under reduced pressure. The crude product was purified by Prep-HPLC with the following conditions: Column: XBridge Shield RP18 OBD Column, 5 um, 19×150 mm; Mobile Phase A: Water (plus 10 mmol/L FA); Mobile Phase B: ACN; Flow rate: 25 mL/min; Gradient: 15% B to 40% B in 7 min; Detector: 254 nm; Rt: 6.38 min) to afford trans-3-(4-[15-[4-([4-[6-(butylamino)-1-[4-hydroxycyclohexyl]-1H-pyrazolo[3,4-d]pyrimidin-3-yl]phenyl]methyl)piperazin-1-yl]-4,7,10,13-tetraoxa-1-azapentadecan-1-yl]-3-methyl-2-oxo-2,3-dihydro-1H-1,3-benzodiazol-1-yl)piperidine-2,6-dione (10 mg, 4%) as a white solid. ¹H NMR (400 MHz, Methanol-d4) δ 8.96 (s, 1H), 7.90 (d, J=8.0 Hz, 2H), 7.47 (d, J=7.9 Hz, 2H), 6.96 (t, J=8.1 Hz, 1H), 6.56 (m, 2H), 5.27 (dd, J=12.2, 5.4 Hz, 1H), 4.71-4.57 (m, 1H), 3.78-3.68 (m, 6H), 3.67-3.54 (m, 16H), 3.51 (t, J=7.1 Hz, 2H), 3.29 (d, J=5.2 Hz, 2H), 2.99-2.84 (m, 2H), 2.82-2.65 (m, 7H), 2.65-2.45 (m, 4H), 2.32-2.20 (m, 2H), 2.20-2.09 (m, 3H), 2.08-1.97 (m, 2H), 1.68 (p, J=7.2 Hz, 2H), 1.61-1.42 (m, 4H), 1.03 (t, J=7.4 Hz, 3H). LC/MS (ESI, m/z): [(M+1)]⁺=940.65

### EXAMPLE 19: trans-3-(5-[1-[4-([4-[6-(butylamino)-1-[4-hydroxycyclohexyl]-1H-pyrazolo[3,4-d]pyrimidin-3-yl]phenyl]methyl)piperazin-1-yl]-3,6,9,12-tetraoxapentadecan-15-yl]-3-methyl-2-oxo-2,3-dihydro-1H-1,3-benzodiazol-1-yl)piperidine-2,6-dione (I-38)

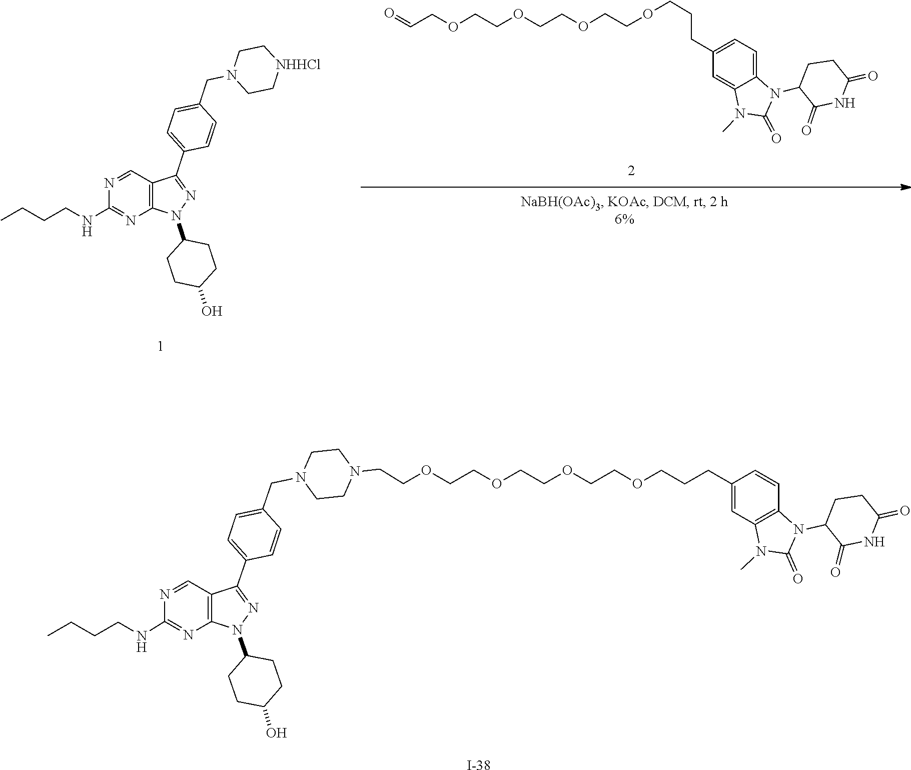

To a stirred solution of trans-4-[6-(butylamino)-3-[4-[(piperazin-1-yl)methyl]phenyl]-1H-pyrazolo[3,4-d]pyrimidin-1-yl]cyclohexan-1-ol hydrochloride (131 mg, 0.26 mmol) in DCM (20 mL) were added 15-[1-(2,6-dioxopiperidin-3-yl)-3-methyl-2-oxo-2,3-dihydro-1H-1,3-benzodiazol-5-yl]-3,6,9,12-tetraoxapentadecanal (129 mg, 0.26 mmol), KOAc (103 mg, 1.05 mmol) and NaBH(OAc)₃ (167 mg, 0.79 mmol) at room temperature. After stirring for an additional 2 h, the resulting mixture was concentrated under reduced pressure. The crude product was purified by reverse phase flash chromatography with the following conditions: Column: XBridge Shield RP18 OBD Column, 5 um, 19×150 mm; Mobile Phase A: Water (plus 10 mmol/L FA); Mobile Phase B: ACN; Flow rate: 25 mL/min; Gradient: 15% B to 40% B in 7 min; Detector: 254 nm; Rt: 6.38 min) to afford trans-3-(5-[1-[4-([4-[6-(butylamino)-1-[4-hydroxycyclohexyl]-1H-pyrazolo[3,4-d]pyrimidin-3-yl]phenyl]methyl)piperazin-1-yl]-3,6,9,12-tetraoxapentadecan-15-yl]-3-methyl-2-oxo-2,3-dihydro-1H-1,3-benzodiazol-1-yl)piperidine-2,6-dione (15 mg, 6%) as a white solid. ¹H NMR (400 MHz, CD₃OD) δ 8.95 (s, 1H), 7.91 (d, J=8.1 Hz, 2H), 7.52-7.43 (d, J=8.1 Hz, 2H), 7.07-6.90 (m, 3H), 5.32 (dd, J=12.6, 5.4 Hz, 1H), 4.70-4.57 (m, 1H), 3.77-3.68 (m, 3H), 3.67-3.59 (m, 12H), 3.55 (m, 2H), 3.49 (t, J=7.1 Hz, 2H), 3.44 (t, J=6.3 Hz, 2H), 3.41 (s, 3H), 3.00-2.60 (m, 15H), 2.30-2.10 (m, 5H), 2.08-1.98 (m, 2H), 1.93-1.82 (m, 2H), 1.75-1.64 (m, 2H), 1.60-1.44 (m, 4H), 1.02 (t, J=7.3 Hz, 3H). LC/MS (ESI, m/z): [(M+1)]⁺=939.50

### EXAMPLE 20: 2-Amino-5-(1-[4-[(3-[3-[1-(2,6-dioxopiperidin-3-yl)-3-methyl-2-oxo-2,3-dihydro-1H-1,3-benzodiazol-5-yl]propoxy]propyl)carbamoyl]butyl]-1H-pyrazol-4-yl)-N-[(1S,2S)-2-([4-[(4-methylpiperazin-1-yl)methyl]-[1,1-biphenyl]-4-yl]methoxy)cyclopentyl]pyridine-3-carboxamide formic acid (I-39)

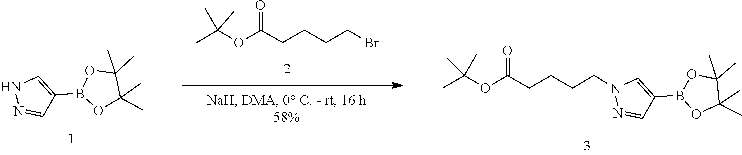 [SMILES: CC(C)(C)OC(=O)CCCCBr ; CC1(C)OB(c2cn[nH]c2)OC1(C)C ; CC(C)(C)OC(=O)CCCCn1cc(B2OC(C)(C)C(C)(C)O2)cn1]

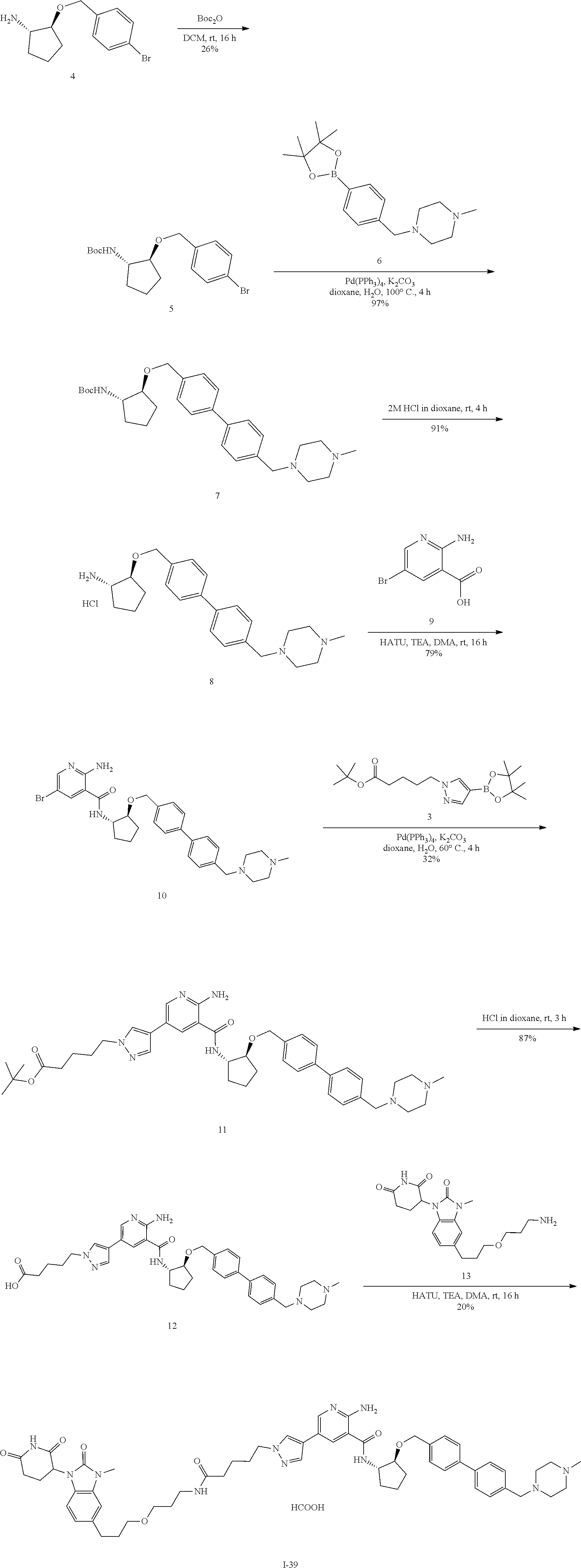

### Step 1: tert-butyl 5-[4-(4,4,5,5-tetramethyl-1,3,2-dioxaborolan-2-yl)-1H-pyrazol-1-yl]pentanoate

A solution of 4-(4,4,5,5-tetramethyl-1,3,2-dioxaborolan-2-yl)-1H-pyrazole (200 mg, 1.03 mmol) in DMF (2 mL) was treated with NaH (60% dispersed in mineral oil, 50 mg, 2.08 mmol) at 0° C. for 1 h under nitrogen atmosphere followed by the addition of tert-butyl 5-bromopentanoate (293 mg, 1.24 mmol). The resulting mixture was stirred for 16 h at room temperature under nitrogen atmosphere. The reaction was quenched by the addition of HOAc (1 mL) at 0° C. The mixture was purified by reverse phase flash chromatography with the following conditions: Column: WelFlash™ C18-I, 20-40 μm, 120 g; Eluent A: Water (plus 10 mmol/L NH₄HCO₃); Eluent B: ACN; Gradient: 60%-80% B in 20 min; Flow rate: 50 mL/min; Detector: 220/254 nm; desired fractions were collected at 74% B and concentrated under reduced pressure to afford tert-butyl 5-[4-(4,4,5,5-tetramethyl-1,3 ,2-dioxaborolan-2-yl)-1H-pyrazol-1-yl]pentanoate (210 mg, 58%) as a light yellow oil. ¹H NMR (400 MHz, DMSO-d₆) δ 7.93 (s, 1H), 7.57 (s, 1H), 4.10 (q, J=5.5, 4.3 Hz, 2H), 2.19 (t, J=7.4 Hz, 2H), 1.75 (p, J=7.0 Hz, 2H), 1.45-1.37 (m, 11H), 1.25 (s, 12H). LC/MS (ESI, m/z): [(M+1)]⁺=351.30

### Step 2: tert-butyl N-[(1S,2S)-2-[(4-bromophenyl)methoxy]yclopentyl]carbamate

To a stirred solution of (1S,2S)-2-[(4-bromophenyl)methoxy]cyclopentan-1-amine (1.4 g, 5.18 mmol) in DCM (20 mL) was added Boc2O (1.36 g, 6.22 mmol) at room temperature under nitrogen atmosphere. The resulting solution was stirred for 16 h at room temperature under nitrogen atmosphere. The resulting solution was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 60%-80% B in 20 min; Flow rate: 80 mL/min; Detector: 220/254 nm; desired fractions were collected at 70% B and concentrated under reduced pressure to afford tert-butyl N-[(1S,2S)-2-[(4-bromophenyl)methoxy]cyclopentyl]carbamate (490 mg, 26%) as a white solid. ¹H NMR (400 MHz, DMSO-d₆) δ 7.52 (d, J=8.4 Hz, 2H), 7.27 (d, J=8.3 Hz, 2H), 6.90 (d, J=7.7 Hz, 1H), 4.56-4.40 (m, 2H), 3.81-3.62 (m, 2H), 1.96-1.72 (m, 2H), 1.68-1.49 (m, 4H), 1.39 (s, 9H). LCMS (ESI, m/z): [(M+1)]⁺=370.10, 372.10

### Step 3: tert-butyl N-[(1S,2S)-2-([4-[(4-methylpiperazin-1-yl)methy]-[1,1-biphenyl]-4-yl]methoxy)cyclopentyl]carbamate

To a stirred solution of tert-butyl N-[(1S,2S)-2-[(4-bromophenyl)methoxy]cyclopentyl]carbamate (200 mg, 0.54 mmol) in dioxane (6 mL) and H₂O (2 mL) were added 1-methyl-4-[[4-(4,4,5,5-tetramethyl-1,3,2-dioxaborolan-2-yl)phenyl]methyl]piperazine (222 mg, 0.71 mmol), K₂CO₃ (224 mg, 1.62 mmol) and Pd(PPh₃)₄ (32 mg, 0.027 mmol) at room temperature. The resulting mixture was stirred for 4 h at 100° C. under nitrogen atmosphere. The resulting mixture was cooled to room temperature, diluted with water (30 mL) and extracted with ethyl acetate (3×30 mL). The combined organic layers was washed with brine (40 mL) and dried over anhydrous Na₂SO₄. After filtration, the filtrate was concentrated under reduced pressure. The residue was purified by reverse flash chromatography with the following conditions: Column: WelFlash™ C18-I, 20-40 μm, 120 g; Eluent A: Water (plus 10 mmol/L NH₄HCO₃); Eluent B: ACN; Gradient: 75%-95% B in 20 min; Flow rate: 50 mL/min; Detector: 220/254 nm; desired fractions were collected at 85% B and concentrated under reduced pressure to afford tert-butyl N-[(1S,2S)-2-([4-[(4-methylpiperazin-1-yl)methyl]-[1,1-biphenyl]-4-yl]methoxy)cyclopentyl]carbamate (250 mg, 97%) as a yellow oil. ¹H NMR (400 MHz, DMSO-d₆) δ 7.65-7.60 (m, 4H), 7.40-7.35 (m, 4H), 6.92 (d, J=7.7 Hz, 1H), 4.63-4.45 (m, 2H), 3.86-3.66 (m, 2H), 3.48 (s, 2H), 2.48-2.24 (m, 8H), 2.15 (s, 3H), 1.96-1.70 (m, 2H), 1.70-1.65 (m, 4H), 1.40 (s, 9H). LC/MS (ESI, m/z): [(M+1)]⁺=480.30

### Step 4: (1S,2S)-2-([4-[(4-methylpiperazin-1-yl)methyl]-[1,1-bipheny]-4-yl]methoxy)cyclopentan-1-amine hydrochloride

To a stirred solution of tert-butyl N-[(1S,2S)-2-([4-[(4-methylpiperazin-1-yl)methyl]-[1,1-biphenyl]-4-yl]methoxy)cyclopentyl]carbamate (250 mg, 0.52 mmol) in 1,4-dioxane (3 mL) was added a solution of hydrocloride in 1,4-dioxane (4 M, 3 mL) at room temperature under nitrogen atmosphere. The resulting mixture was stirred for 4 h at room temperature. The resulting mixture was concentrated under reduced pressure. The residue was triturated with diethyl ether (20 mL) and the precipitated solids were collected by filtration, dried in a vacuum oven to afford (1S,2S)-2-([4-[(4-methylpiperazin-1-yl)methyl]-[1,1-biphenyl]-4-yl]methoxy)cyclopentan-1-amine hydrochloride (197 mg, 91%) as a yellow solid. ¹H NMR (400 MHz, DMSO-d₆) δ 8.28 (br s, 3H), 7.83-7.64 (m, 6H), 7.48 (d, J=8.1 Hz, 2H), 4.58 (s, 2H), 4.04-3.98 (m, 1H), 3.95-3.70 (m, 4H), 3.70-3.51 (m, 4H), 3.50-3.31 (m, 3H), 2.79 (s, 3H), 2.12-1.94 (m, 2H), 1.78-1.55 (m, 4H). LC/MS (ESI, m/z): [(M+1)]⁺=380.30

### Step 5: 2-Amino-5-bromo-N-[(1S,2S)-2-([4-[(4-methylpiperazin-1-yl)methy]-[1,1-biphenyl]-4-yl]methoxy)cyclopentyl]pyridine-3-carboxamide

To a stirred solution of 2-amino-5-bromopyridine-3-carboxylic acid (125 mg, 0.58 mmol) in DMA (2 mL) were added HATU (238 mg, 0.63 mmol), TEA (146 mg, 1.44 mmol) and (1S,2S)-2-([4-[(4-methylpiperazin-1-yl)methyl]-[1,1-biphenyl]-4-yl]methoxy)cyclopentan-1-amine hydrochloride (200 mg, 0.48 mmol). The resulting mixture was stirred for 16 h at room temperature under nitrogen atmosphere. The resulting mixture was purified by reverse phase flash chromatography with the following conditions: Column: WelFlash™ C18-I, 20-40 μm, 120 g; Eluent A: Water (plus 10 mmol/L formic acid); Eluent B: ACN; Gradient: 25%-45% B in 20 min; Flow rate: 50 mL/min; Detector: 220/254 nm; desired fractions were collected at 35% B and concentrated under reduced pressure to afford 2-amino-5-bromo-N-[(1S,2S)-2-([4-[(4-methylpiperazin-1-yl)methyl]-[1,1-biphenyl]-4-yl]methoxy)cyclopentyl]pyridine-3-carboxamide (220 mg, 79%) as a yellow solid. ¹H NMR (400 MHz, DMSO-d₆) δ 8.43 (d, J=7.6 Hz, 1H), 8.15 (d, J=2.6 Hz, 1H), 8.09 (dd, J=7.7, 2.5 Hz, 1H), 7.65-7.60 (m, 4H), 7.43-7.39 (m, 4H), 7.24 (s, 2H), 4.66-4.53 (m, 2H), 4.27 (dt, J=11.4, 7.3 Hz, 1H), 3.90 (dt, J=6.2, 3.6 Hz, 1H), 3.61 (s, 2H), 3.16-2.86 (m, 8H), 2.65 (s, 3H), 2.13-1.87 (m, 2H), 1.81-1.62 (m, 3H), 1.57 (dt, J=13.8, 6.7 Hz, 1H). LC/MS (ESI, m/z): [(M+1)]⁺=578.20, 580.20

### Step 6: tert-butyl 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoate

To a stirred solution of 2-amino-5-bromo-N-[(1S,2S)-2-([4-[(4-methylpiperazin-1-yl)methyl]-[1,1-biphenyl]-4-yl]methoxy)cyclopentyl]pyridine-3-carboxamide (200 mg, 0.35 mmol) in 1,4-dioxane (6 mL) were added H₂O (2 mL), tert-butyl 5-[4-(4,4,5,5-tetramethyl-1,3,2-dioxaborolan-2-yl)-1H-pyrazol-1-yl]pentanoate (158 mg, 0.45 mmol), K₂CO₃ (144 mg, 1.04 mmol) and Pd(PPh₃)₄ (20 mg, 0.017 mmol) at room temperature under nitrogen atmosphere. The resulting mixture was stirred for 4 h at 60° C. After cooling down to room temperature, the resulting mixture was diluted with water (10 mL) and extracted with ethyl acetate (3×20 mL). The combined organic layers was washed with brine (30 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 120 g; Eluent A: Water (plus 10 mmol/L NH₄HCO₃); Eluent B: ACN; Gradient: 70%-95% B in 30 min; Flow rate: 50 mL/min; Detector: 220/254 nm; desired fractions were collected at 85% B and concentrated under reduced pressure to afford tert-butyl 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoate (80 mg, 32%) as a white solid. ¹H NMR (400 MHz, DMSO-d₆) δ 8.37 (d, J=7.6 Hz, 1H), 8.33 (d, J=2.5 Hz, 1H), 8.04 (s, 1H), 8.00 (s, 1H), 7.81 (s, 1H), 7.63-7.51 (m, 4H), 7.40 (d, J=7.7 Hz, 2H), 7.35 (d, J=7.7 Hz, 2H), 6.99 (s, 2H), 4.61 (d, J=3.3 Hz, 2H), 4.31 (s, 1H), 4.09 (t, J=6.5 Hz, 2H), 3.92 (s, 1H), 3.48 (s, 2H), 2.34 (s, 7H), 2.21 (t, J=7.4 Hz, 2H), 2.15 (s, 3H), 2.11-1.99 (m, 1H), 1.99-1.88 (m, 1H), 1.84-1.64 (m, 6H), 1.59 (dd, J=13.6, 6.6 Hz, 1H), 1.45 (q, J=7.5 Hz, 2H), 1.37 (d, J=1.8 Hz, 9H). LC/MS (ESI, m/z): [(M+1)]⁺=722.45

### Step 7: 5-[4-(6-Amino-5-[[(1S,2S)-2-([4-[(4-methylpiperazin-1-yl)methyl]-[1,1-bipheny]-4-yl]methoxy)cyclopentyl]carbamoyl]pyridin-3-yl)-1H-pyrazol-1-yl]pentanoic acid

To a stirred solution of tert-butyl 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoate (100 mg, 0.139 mmol) in 1,4-dioxane (6 mL) was added a solution of hydrochloride in 1,4-dioxane (4 M, 2 mL) at room temperature under nitrogen atmosphere. The resulting solution was stirred for 3 h at room temperature. The resulting solution was concentrated under reduced pressure to afford 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoic acid (80 mg, 87%) as a yellow solid. ¹H NMR (400 MHz, DMSO-d₆) δ 11.92 (br s, 1H), 9.26 (d, J=7.6 Hz, 1H), 8.81 (d, J=2.0 Hz, 1H), 8.60-8.26 (m, 4H), 8.05 (s, 1H), 7.78-7.69 (m, 4H), 7.65 (d, J=8.0 Hz, 2H), 7.43 (d, J=8.1 Hz, 2H), 4.62 (s, 2H), 4.44 (s, 2H), 4.31 (s, 1H), 4.12 (d, J=6.9 Hz, 2H), 4.06 (dd, J=6.8, 3.4 Hz, 1H), 3.77-3.33 (m, 8H), 2.81 (s, 3H), 2.25 (t, J=7.4 Hz, 2H), 2.12-1.92 (m, 2H), 1.87-1.63 (m, 6H), 1.46 (p, J=7.4 Hz, 2H). LC/MS (ESI, m/z): [(M+1)]⁺=666.50

### Step 8: 2-Amino-5-(1-[4-[(3-[3-[1-(2,6-dioxopiperidin-3-yl)-3-methyl-2-oxo-2,3-dihydro-1H-1,3-benzodiazol-5-yl]propoxy]propyl)carbamoyl]butyl]-1H-pyrazol-4-yl)-N-[(1S,2S)-2-([4-[(4-methylpiperazin-1-yl)methy]-[1,1-bipheny]-4-yl]methoxy)cyclopentyl]pyridine-3-carboxamide formic acid

To a stirred solution of 3-[5-[3-(3-aminopropoxy)propyl]-3-methyl-2-oxo-2,3-dihydro-1H-1,3-benzodiazol-1-yl]piperidine-2,6-dione hydrochloride (25 mg, 0.06 mmol) in DMA (2 mL) were added TEA (19 mg, 0.18 mmol), 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoic acid (40 mg, 0.06 mmol) and HATU (20 mg, 0.078 mmol) at 0° C. under nitrogen atmosphere. The resulting mixture was stirred for 16 h at room temperature under nitrogen atmosphere. The resulting mixture was purified by reverse phase flash chromatography with the following conditions: Column: WelFlash™ C18-I, 20-40 μm, 120 g; Eluent A: Water (plus 10 mmol/L formic acid); Eluent B: ACN; Gradient: 25%-45% B in 20 min; Flow rate: 50 mL/min; Detector: 220/254 nm; desired fractions were collected at 36% B and concentrated under reduced pressure to afford 2-amino-5-(1-[4-[(3-[3-[1-(2,6-dioxopiperidin-3-yl)-3-methyl-2-oxo-2,3-dihydro-1H-1,3-benzodiazol-5-yl]propoxy]propyl)carbamoyl]butyl]-1H-pyrazol-4-yl)-N-[(1S,2S)-2-([4-[(4-methylpiperazin-1-yl)methyl]-[1,1-biphenyl]-4-yl]methoxy)cyclopentyl]pyridine-3-carboxamide formic acid (13 mg, 20%) as a white solid. ¹H NMR (400 MHz, CD₃OD) δ 8.51 (s, 1H), 8.26 (d, J=2.3 Hz, 1H), 8.01 (d, J=2.3 Hz, 1H), 7.89 (s, 1H), 7.78 (s, 1H), 7.53 (m, 4H), 7.41 (m, 4H), 7.03-6.96 (m, 2H), 6.92 (dd, J=8.0, 1.5 Hz, 1H), 5.30 (dd, J=12.6, 5.5 Hz, 1H), 4.73-4.59 (m, 2H), 4.47-4.38 (m, 1H), 4.13 (t, J=6.9 Hz, 2H), 4.03-3.95 (m, 1H), 3.66 (s, 2H), 3.48-3.41 (m, 2H), 3.39 (m, 5H), 3.26 (t, J=6.9 Hz, 2H), 3.07-2.86 (m, 5H), 2.85-2.75 (m, 2H), 2.74-2.66 (m, 4H), 2.65 (s, 3H), 2.25-2.11 (m, 4H), 2.09-1.99 (m, 1H), 1.92-1.71 (m, 10H), 1.67-1.53 (m, 4H). LC/MS (ESI, m/z): [(M+1)]⁺=1022.55

### EXAMPLE 21: 3-(5-[3-[2-(2-[2-[4-([4-[2-(Butylamino)-7-[trans-4-hydroxycyclohexyl]-7H-pyrrolo[2,3-d]pyrimidin-5-yl]phenyl]methyl)piperazin-1-yl]ethoxy]ethoxy)ethoxy]propyl]-3-methyl-2-oxo-2,3-dihydro-1H-1,3-benzodiazol-1-yl)piperidine-2,6-dione formic acid (I-40)

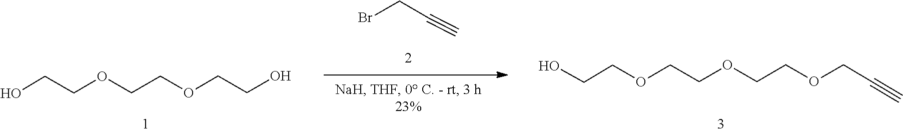

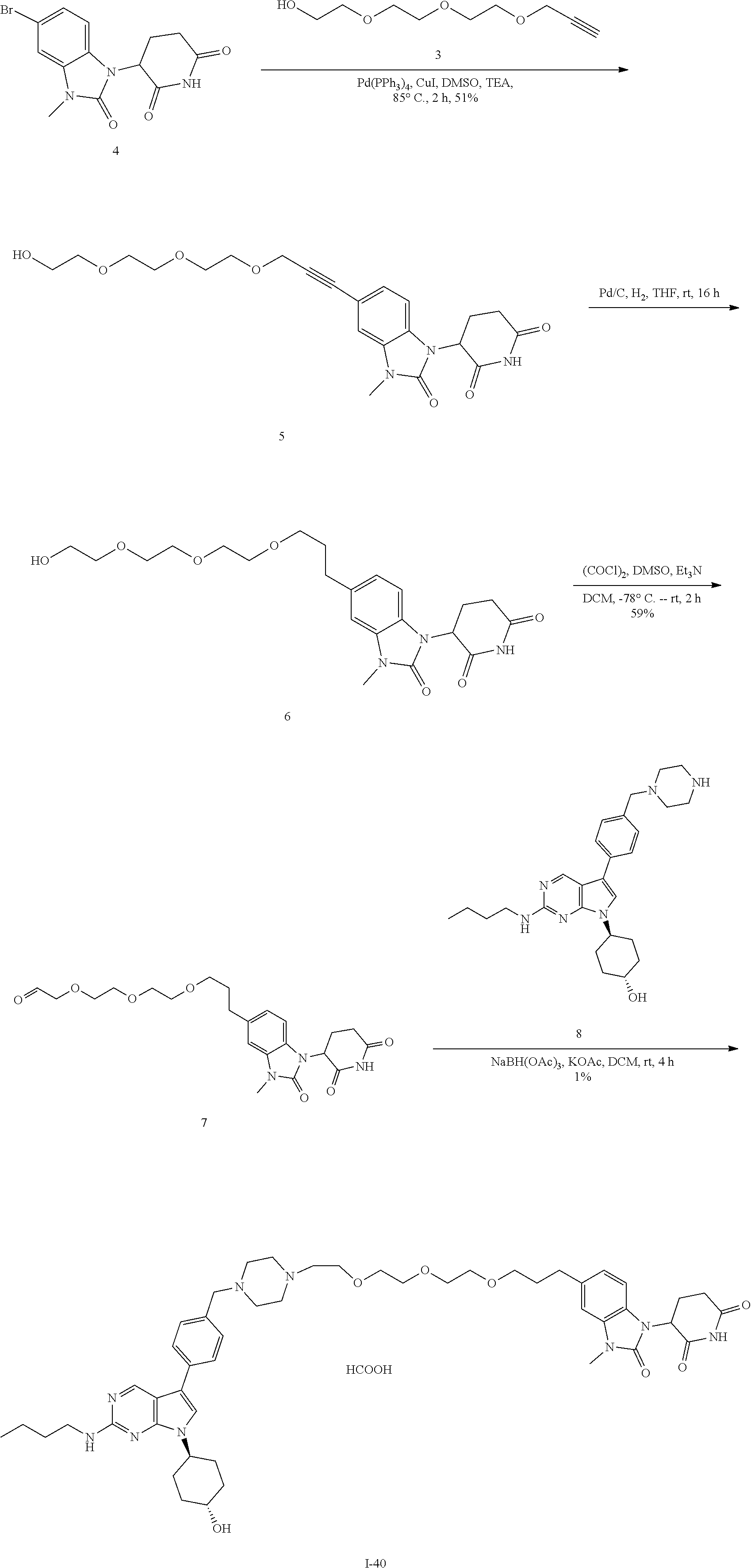

### Step 1: 2-[2-[2-(Prop-2-yn-1-yloxy)ethoxy]ethoxy]ethan-1-ol

To a stirred solution of 2-[2-(2-hydroxyethoxy)ethoxy]ethan-1-ol (50 g, 331 mmol) in THF (1 L) was added NaH (60% dispersion in mineral oil, 13.3 g, 554 mmol) over 10 min at 0° C. To the above mixture was added 3-bromoprop-1-yne (20 g, 166 mmol) over 10 min at 0° C. under nitrogen atmosphere. The resulting mixture was stirred for 3 h at room temperature under nitrogen atmosphere. The reaction was quenched with a saturated aqueous solution of ammonium chloride (200 mL). The resulting mixture was extracted with ethyl acetate (2×1 L). The combined organic layers was washed with brine (1 L) and dried over anhydrous Na₂SO₄. After filtration, the filtrate was concentrated under reduced pressure. The residue was purified by silica gel column chromatography, eluted with ethyl acetate to afford 2-[2-[2-(prop-2-yn-1-yloxy)ethoxy]ethoxy]ethan-1-ol (7.3 g, 23%) as a light yellow oil. ¹H NMR (400 MHz, CDCl₃) δ 4.16 (d, J=2.3 Hz, 2H), 3.71-3.59 (m, 10H), 3.62-3.52 (m, 2H), 2.88 (s, 1H), 2.43 (t, J=2.4 Hz, 1H). LC/MS (ESI, m/z): [(M−1)]⁻=187.05

### Step 2: 3-[5-(3-[2-[2-(2-Hydroxyethoxy)ethoxy]ethoxy]prop-1-yn-1-yl)-3-methyl-2-oxo-2,3-dihydro-1H-1,3-benzodiazol-1-yl]piperidine-2,6-dione

To a stirred solution of 3-(5-bromo-3-methyl-2-oxo-2,3-dihydro-1H-1,3-benzodiazol-1-yl)piperidine-2,6-dione (2 g, 5.91 mmol) in DMSO (15 mL) were added TEA (7.5 mL), CuI (90 mg, 0.47 mmol), 2-[2-[2-(prop-2-yn-1-yloxy)ethoxy]ethoxy]ethan-1-ol (5.57 g, 29.57 mmol) and Pd(PPh₃)₄ (683 mg, 0.59 mmol) at room temperature. The resulting mixture was stirred for 2 h at 85° C. under nitrogen atmosphere. After cooling down to room temperature, the resulting mixture was purified by reverse flash chromatography with the following conditions: Column: WelFlash™ C18-I, 20-40 μm, 330 g; Eluent A: Water (plus 10 mmol/L formic acid); Eluent B: ACN; Gradient: 20%-40% B in 20 min; Flow rate: 80 mL/min; Detector: 220/254 nm; desired fractions were collected at 26% B and concentrated under reduced pressure to afford 3-[5-(3-[2-[2-(2-hydroxyethoxy)ethoxy]ethoxy]prop-1-yn-1-yl)-3-methyl-2-oxo-2,3-dihydro-1H-1,3-benzodiazol-1-yl]piperidine-2,6-dione (1.35 g, 51%) as a yellow solid. ¹H NMR (400 MHz, DMSO-d₆) δ 11.14 (s, 1H), 7.35 (d, J=1.4 Hz, 1H), 7.22-7.11 (m, 2H), 5.40 (dd, J=12.7, 5.3 Hz, 1H), 4.60 (s, 1H), 4.40 (s, 2H), 3.64 (dd, J=4.4, 1.9 Hz, 2H), 3.61-3.56 (m, 2H), 3.55-3.43 (m, 6H), 3.42 (t, J=4.9 Hz, 2H), 3.35 (s, 3H), 2.96-2.83 (m, 1H), 2.73 (td, J=12.8, 4.4 Hz, 1H), 2.63 (d, J=18.5 Hz, 1H), 2.11-1.99 (m, 1H). LC/MS (ESI, m/z): [(M+1)]⁺=446.15

### Step 3: 3-[5-(3-[2-[2-(2-Hydroxyethoxy)ethoxy]ethoxy]propyl)-3-methyl-2-oxo-2,3-dihydro-1H-1,3-benzodiazol-1-yl]piperidine-2,6-dione

To a stirred solution of 3-[5-(3-[2-[2-(2-hydroxyethoxy)ethoxy]ethoxy]prop-1-yn-1-yl)-3-methyl-2-oxo-2,3-dihydro-1H-1,3-benzodiazol-1-yl]piperidine-2,6-dione (1.35 g, 3.03 mmol) in THF (20 mL) was added palladium on charcoal (135 mg, 10% w/w) at room temperature. The resulting mixture was purged with hydrogen three times and stirred for overnight at room temperature under hydrogen atmosphere. The resulting mixture was filtered. The filter cake was washed with EtOAc (3×10 mL). The combined filtrate was concentrated under reduced pressure to afford 3-[5-(3-[2-[2-(2-hydroxyethoxy)ethoxy]ethoxy]propyl)-3-methyl-2-oxo-2,3-dihydro-1H-1,3-benzodiazol-1-yl]piperidine-2,6-dione (1.21 g, 89%) as a yellow oil. ¹H NMR (400 MHz, DMSO-d₆) δ 11.10 (s, 1H), 7.08-6.98 (m, 2H), 6.88 (dd, J=8.1, 1.6 Hz, 1H), 5.34 (dd, J=12.7, 5.4 Hz, 1H), 4.59 (t, J=5.5 Hz, 1H), 3.57-3.46 (m, 10H), 3.49-3.36 (m, 4H), 3.33 (s, 3H), 2.90 (ddd, J=16.6, 13.1, 5.3 Hz, 1H), 2.78-2.58 (m, 4H), 2.00 (q, J=4.8 Hz, 1H), 1.88-1.74 (m, 2H). LC/MS (ESI, m/z): [(M+1)]⁺=450.35

### Step 4: 2-[2-(2-[3-[1-(2,6-Dioxopiperidin-3-yl)-3-methyl-2-oxo-2,3-dihydro-1H-1,3-benzodiazol-5-yl]propoxy]ethoxy)ethox]acetaldehyde

To a stirred solution of (COCl)₂ (1.01 g, 8.01 mmol) in DCM (10 mL) was added a solution of DMSO (834 mg, 10.67 mmol) −78° C. under nitrogen atmosphere. The resulting solution was stirred for 0.5 h at −60° C. under nitrogen atmosphere. To the above solution was added a solution of 3-[5-(3-[2-[2-(2-hydroxyethoxy)ethoxy]ethoxy]propyl)-3-methyl-2-oxo-2,3-dihydro-1H-1,3-benzodiazol-1-yl]piperidine-2,6-dione (1.2 g, 2.67 mmol) in DCM (10 mL) dropwise at −60° C. The resulting solution was stirred for 1 h at −60° C. under nitrogen atmosphere. To the above solution was added TEA (2.16 g, 21.36 mmol) dropwise at −60° C. The resulting solution was stirred for 1 h at room temperature under nitrogen atmosphere. The resulting mixture was concentrated under reduced pressure. The residue was purified by reverse flash chromatography with the following conditions: Column: WelFlash™ C18-I, 20-40 μm, 330 g; Eluent A: Water (plus 10 mmol/L HOAc); Eluent B: ACN; Gradient: 15%-35% B in 20 min; Flow rate: 80 mL/min; Detector: 220/254 nm; desired fractions were collected at 29% B and concentrated under reduced pressure to afford 2-[2-(2-[3-[1-(2,6-dioxopiperidin-3-yl)-3-methyl-2-oxo-2,3-dihydro-1H-1,3-benzodiazol-5-yl]propoxy]ethoxy)ethoxy]acetaldehyde (700 mg, 59%) as a yellow solid. ¹H NMR (400 MHz, DMSO-d₆) δ 11.11 (s, 1H), 9.58 (s, 1H), 7.08-6.97 (m, 2H), 6.88 (dd, J=8.0, 1.6 Hz, 1H), 5.35 (dd, J=12.8, 5.4 Hz, 1H), 4.20 (s, 2H), 3.67-3.45 (m, 9H), 3.44-3.33 (m, 4H), 2.97-2.84 (m, 1H), 2.78-2.56 (m, 4H), 2.01 (ddt, J=11.3, 6.0, 3.0 Hz, 1H), 1.82 (dq, J=8.9, 6.5 Hz, 2H). LC/MS (ESI, m/z): [(M+1)]⁺=448.35

### Step 5: 3-(5-[3-[2-(2-[2-[4-([4-[2-(Butylamino)-7-[trans-4-hydroxycyclohexyl]-7H-pyrrolo[2,3-d]pyrimidin-5-yl]phenyl]methyl)piperazin-1-yl]ethoxy]ethoxy)ethoxy]propyl]-3-methyl-2-oxo-2,3-dihydro-1H-1,3-benzodiazol-1-yl)piperidine-2,6-dione formic acid

To a stirred solution of (trans-4-[2-(butylamino)-5-[4-[(piperazin-1-yl)methyl]phenyl]-7H-pyrrolo[2,3-d]pyrimidin-7-yl]cyclohexan-1-ol hydrochloride (781 mg, 1.56 mmol) in DCM (20 mL) were added KOAc (614 mg, 6.26 mmol), 2-[2-(2-[3-[1-(2,6-dioxopiperidin-3-yl)-3-methyl-2-oxo-2,3-dihydro-1H-1,3-benzodiazol-5-yl]propoxy]ethoxy)ethoxy]acetaldehyde (700 mg, 1.56 mmol) and NaBH(AcO)₃ (1.33 g, 6.26 mmol) at 0° C. under nitrogen atmosphere. The resulting mixture was stirred for 4 h at room temperature under nitrogen atmosphere. The resulting mixture was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 30%-60% B in 30 min; Flow rate: 80 mL/min; Detector: 220/254 nm; desired fractions were collected at 48% B, acidified with formic acid and concentrated under reduced pressure to afford 3-(5-[3-[2-(2-[2-[4-([4-[2-(butylamino)-7-[trans-4-hydroxycyclohexyl]-7H-pyrrolo[2,3-d]pyrimidin-5-yl]phenyl]methyl)piperazin-1-yl]ethoxy]ethoxy)ethoxy]propyl]-3-methyl-2-oxo-2,3-dihydro-1H-1,3-benzodiazol-1-yl)piperidine-2,6-dione formic acid (20 mg, 1%) as a yellow solid. ¹H NMR (400 MHz, CD₃OD) δ 8.70 (s, 1H), 8.44 (s, 1H), 7.62 (d, J=7.8 Hz, 2H), 7.49-7.30 (m, 3H), 7.10-6.84 (m, 3H), 5.30 (dd, J=12.7, 5.4 Hz, 1H), 4.45 (p, J=8.4 Hz, 1H), 3.77 (q, J=5.3 Hz, 2H), 3.74-3.62 (m, 8H), 3.58 (dt, J=6.5, 3.0 Hz, 2H), 3.50-3.40 (m, 4H), 3.38 (s, 3H), 3.35-3.33 (m, 2H), 3.27-3.05 (m, 5H), 2.98-2.64 (m, 9H), 2.1902.08 (m, 3H), 2.21-2.09 (m, 4H), 1.92-1.80 (m, 2H), 1.67 (p, J=7.3 Hz, 2H), 1.60-1.41 (m, 4H), 1.02 (t, J=7.3 Hz, 3H). LC/MS (ESI, m/z): [(M+1)]⁺=894.65

### EXAMPLE 22: 4-((14-(4-((4-(6-(butylamino)-1-(trans-4-hydroxycyclohexyl)-1H-pyrazolo[3,4-d]pyrimidin-3-yl)phenyl)sulfonyl)piperazin-1-yl)-3,6,9,12-tetraoxatetradecyl)amino)-2-(2,6-dioxopiperidin-3-yl)isoindoline-1,3-dione (I-34)

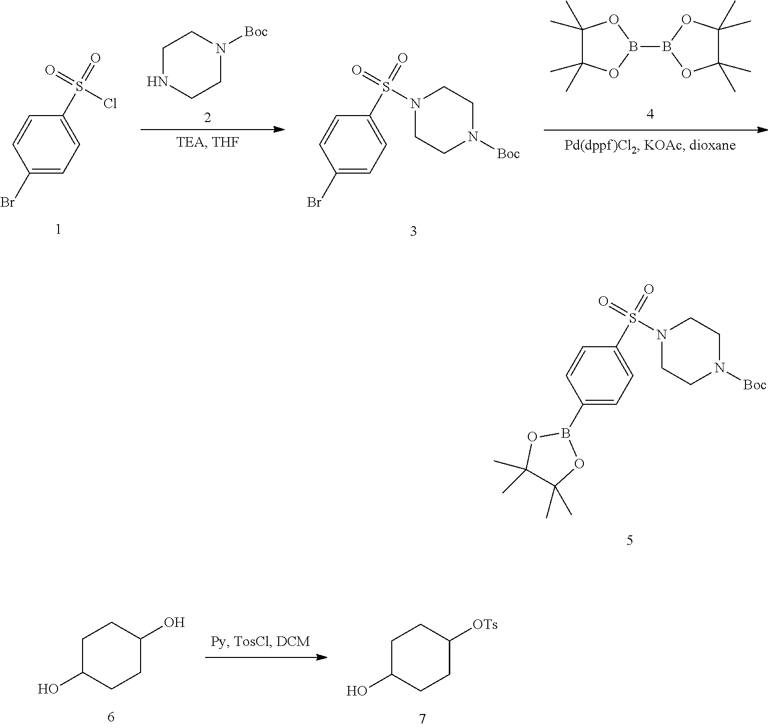

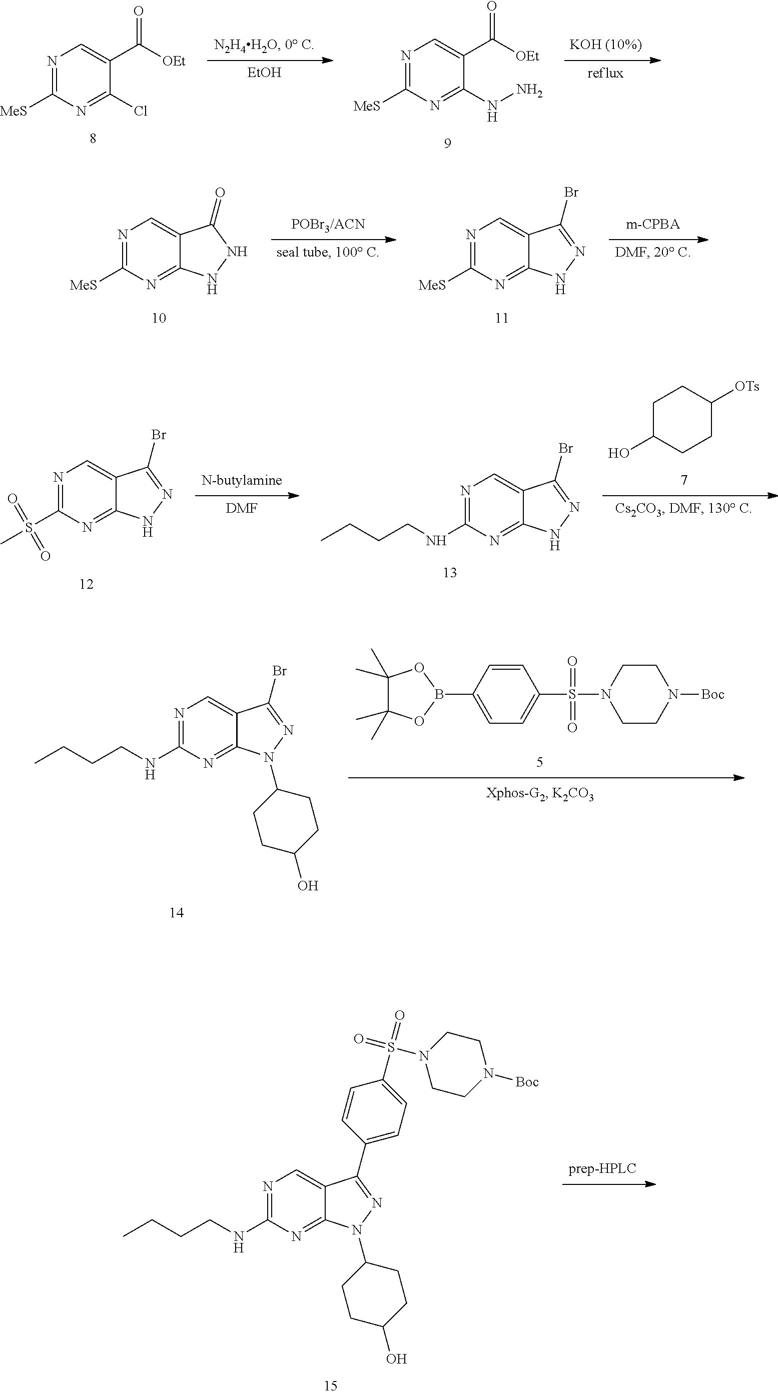

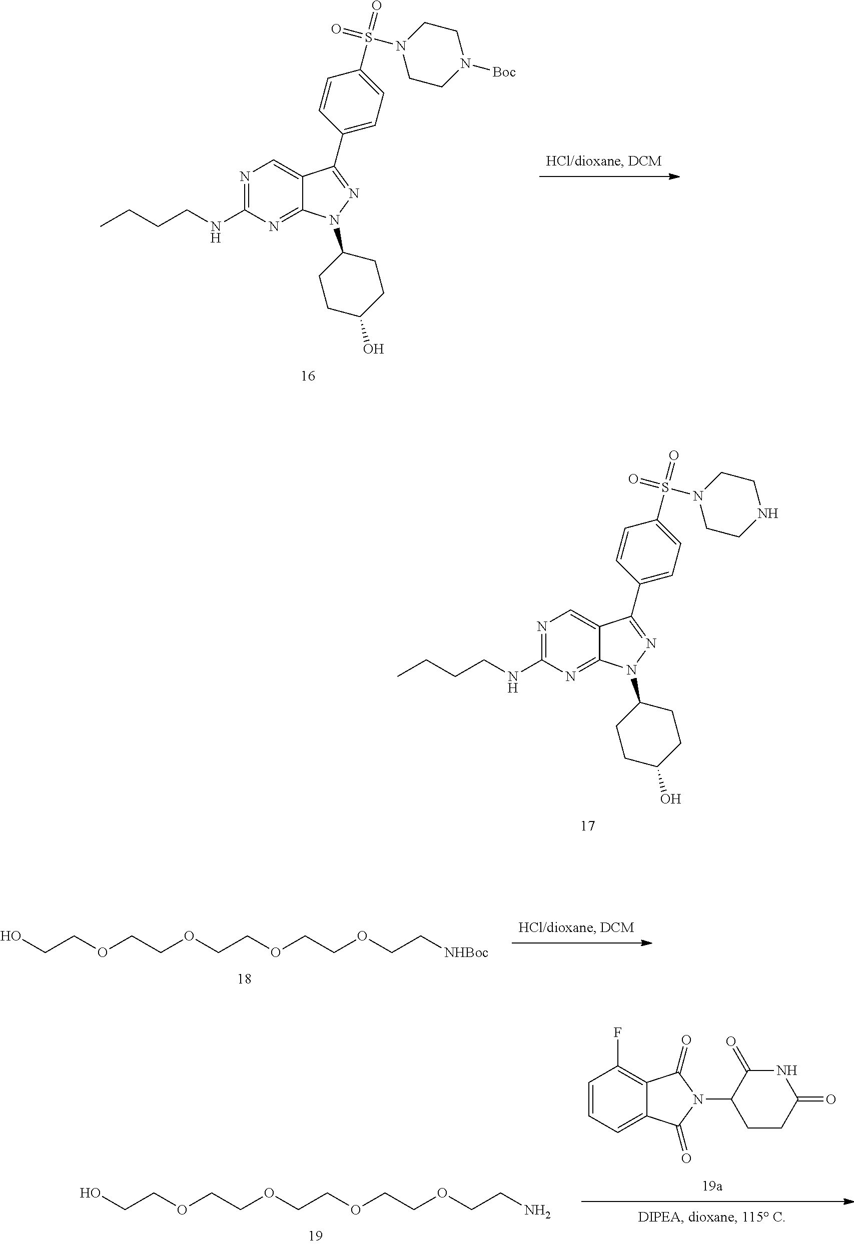 [SMILES: CCCCNc1ncc2c(-c3ccc(S(=O)(=O)N4CCN(C(=O)OC(C)(C)C)CC4)cc3)nn([C@H]3CC[C@H](O)CC3)c2n1 ; CCCCNc1ncc2c(-c3ccc(S(=O)(=O)N4CCNCC4)cc3)nn([C@H]3CC[C@H](O)CC3)c2n1 ; CC(C)(C)OC(=O)NCCOCCOCCOCCOCCO ; O=C1CCC(N2C(=O)c3cccc(F)c3C2=O)C(=O)N1 ; NCCOCCOCCOCCOCCO]

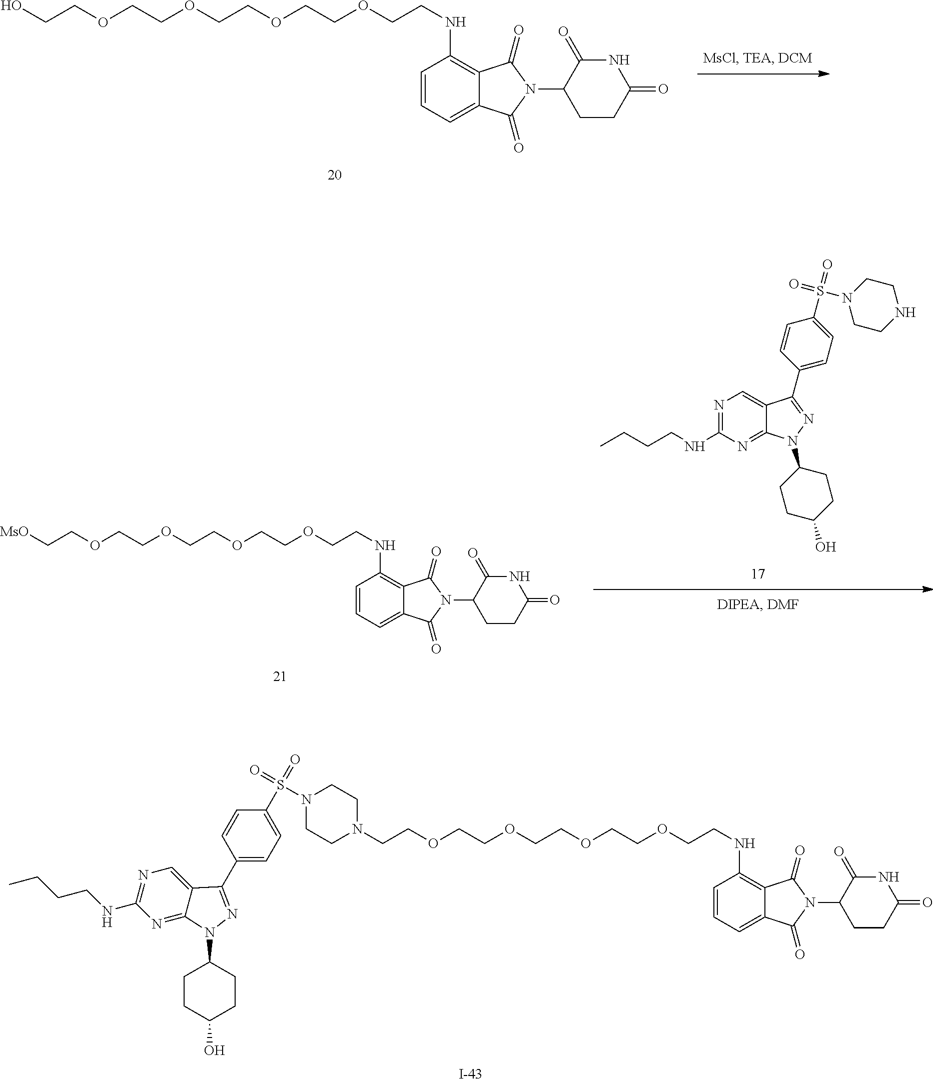

### Step 1: tert-butyl 4-((4-bromophenyl)sulfonyl)piperazine-1-carboxylate (3)

To a solution of tert-butyl piperazine-1-carboxylate (20.1 g, 108 mmol) in THF (250 mL) was added TEA (19.8 g, 196 mmol). The mixture was stirred at 0° C. for 10 minutes. Then 4-bromobenzene sulfonylchloride (25.0 g, 97.8 mmol) was added to the reaction mixture, the reaction mixture was stirred at 25° C. for 50 minutes. On completion, the reaction mixture was filtered and concentrated in vacuo. The residue was washed with water (100 mL) and 10% HCl (50 mL), and then extracted with DCM (2×50 mL). The organic layer was washed with NaHCO₃ (50 mL) to pH=8. The combined organic layers were dried over Na₂SO_{4,} filtered and concentrated in vacuo to give a residue. The residue was purified by column chromatography (petroleum ether/ethyl acetate=0:1) to give the title compound (33 g, 83% yield)) as a white solid. ¹H NMR (400 MHz, CDCl₃) δ 7.72-7.70 (m, 2H), 7.64-7.62 (m, 2H), 3.54 (t, J=4.8 Hz, 4H), 3.00 (t, J=4.8 Hz, 4H), 1.43 (s, 9H)

### Step 2: tert-butyl 4-((4-(4,4,5,5-tetramethyl-1,3,2-dioxaborolan-2-yl)phenyl)sulfonyl)piperazine-1-carboxylate (5)

To a solution of tert-butyl 4-(4-bromophenyl)sulfonylpiperazine-1-carboxylate (10.0 g, 24.7 mmol) and 4,4,5,5-tetramethyl-2-(4,4,5,5-tetramethyl-1,3,2-dioxaborolan-2-yl)-1,3,2-dioxaborolane (18.8 g, 74.0 mmol) in dioxane (100mL) was added Pd(dppf)Cl₂ (1.81 g, 2.47 mmol) and KOAc (4.84 g, 49.4 mmol). The reaction mixture was stirred at 80° C. for 3 hours. On completion, the mixture was filtered, and the filtrate was concentrated in vacuo to give a residue. The residue was purified by flash silica gel chromatography (Ethyl acetate: Petroleum ether=1/1) to give the title compound (8.00 g, 63% yield) as a gray solid. ¹H NMR (400 MHz, CDCl₃) δ 7.98 (d, J=8.0 Hz, 2H), 7.74 (d, J=8.0 Hz, 2H), 3.52 (t, J=4.8 Hz, 4H), 2.98 (t, J=4.8 Hz, 4H), 1.42 (s, 9H), 1.37 (s, 12H), LC-MS (ESI⁺) m/z 397.0 (M+H−56)⁺

### Step 3: (4-Hydroxycyclohexyl) 4-methylbenzenesulfonate (7)

To a solution of Py (12.3 g, 155 mmol) in CHCl₃ (150 mL) was added cyclohexane-1,4-diol (15.0 g, 129 mmol) at 25° C. Then the mixture was cooled to 0° C. and 4-methylbenzenesulfonyl chloride (24.6 g, 129 mmol) was added into the mixture. The reaction mixture was stirred at 25° C. for 18 hrs. On completion, the reaction mixture was acidified with 1N HCl solution until pH=5-6, extracted with EA (3×30 mL). The organic layer was washed with brine (50 mL), dried over Na₂SO_{4,} concentrated in vacuo. The residue was purified by silica gel chromatography to give the title compound (16.0 g, 46% yield) as white solid. ¹H NMR (400 MHz, CDCl₃) δ 7.85-7.76 (m, 2H), 7.36 (d, J=8.0 Hz, 2H), 4.66-4.49 (m, 1H), 3.80-3.70 (m, 1H), 2.47 (s, 3H), 1.97-1.91 (m, 2H), 1.72-1.65 (m, 2H), 1.64-1.50 (m, 4H)

### Step 4: ethyl 4-hydrazinyl-2-(methylthio)pyrimidine-5-carboxylate (9)

To a solution of N₂H₄.H₂O (3.24 g, 63.4 mmol, 3.14 mL, 98% purity) in EtOH (50 mL) was added a solution of ethyl 4-chloro-2-methylsulfanyl-pyrimidine-5-carboxylate (5.00 g, 21.5 mmol) in EtOH (150 mL). The mixture was stirred at 0° C. for 1 hour. On completion, the mixture was concentrated in vacuo to give the title compound (4.91 g, 100% yield) as a yellow solid. The crude product was used to the next step directly without further purification. LC-MS (ESI⁺) m/z 229.0 (M+H)⁺

### Step 5: 6-(Methylthio)-1H-pyrazolo[3,4-d]pyrimidin-3(2H)-one (10)

Ethyl 4-hydrazino-2-methylsulfanyl-pyrimidine-5-carboxylate (4.91 g, 21.5 mmol) was added to a solution of KOH (5 g, 89.12 mmol) in H₂O (45 mL). Then the reaction mixture was stirred at 100° C. for 30 minutes. On completion, the mixture was cooled to 0° C., the mixture was acidified with 25% AcOH solution until pH=3. The mixture was filtered; the filtered cake was collected and concentrated in vacuo to give the title compound (2.50 g, 64% yield) as a yellow solid. ¹H NMR (400 MHz, DMSO-d₆) δ 11.34 (s, 2H), 8.87 (s, 1H), 2.52 (s, 3H)

### Step 6: 3-Bromo-6-(methylthio)-1H-pyrazolo[3,4-d]pyrimidine (11)

To a solution of 6-methylsulfanyl-1,2-dihydropyrazolo[3,4-d]pyrimidin-3-one (2.50 g, 13.7 mmol) in ACN (75 mL) was added POBr₃ (8.26 g, 28.8 mmol, 2.93 mL). The mixture was stirred at 100° C. for 60 hours under seal tube. On completion, the mixture was diluted with H₂O (50 mL), the mixture was basified with NH₃.H₂O until pH=8 under 0° C., the mixture was stirred at 0° C. for 1 hour. The mixture was concentrated in vacuo, and then extracted with EA (3×30mL). The combined organic layers was washed with brine (2×100 mL), dried with anhydrous Na₂SO₄, filtered and concentrated in vacuo to give the title compound (1.60 g, 44% yield). ¹H NMR (400 MHz, DMSO-d₆) δ 14.25 (s, 1H), 9.02 (s, 1H), 2.58 (s, 3H)

### Step 7: 3-Bromo-6-(methylsulfonyl)-1H-pyrazolo[3,4-d]pyrimidine (12)

To a solution of 3-bromo-6-methylsulfanyl-1H-pyrazolo[3,4-d]pyrimidine (1.60 g, 6.53 mmol) in DMF (40 mL) was added m-CPBA (3.00 g, 13.1 mmol, 75% purity). The mixture was stirred at 25° C. for 2 hours. On completion, the mixture was concentrated in vacuo to give the title compound (1.81 g, 100% yield) as a yellow solid. The crude product was used to the next step directly without further purification. LC-MS (ESI⁺) m/z 276.9 (M+H)⁺

### Step 8: 3-Bromo-N-butyl-1H-pyrazolo[3,4-d]pyrimidin-6-amine (13)

To a solution of 3-bromo-6-methylsulfonyl-1H-pyrazolo[3,4-d]pyrimidine (1.81 g, 6.53 mmol) in DMF (20 mL) was added N-butylamine (2.39 g, 32.7 mmol). The mixture was stirred at 25° C. for 14 hours. On completion, the mixture was diluted with water (120 mL) and stirred for 1 hour, filtered and the solid was concentrated in vacuo to give the title compound (800 mg, 45% yield) as a yellow solid. ¹H NMR (400 MHz, DMSO-d₆) δ 13.32 (s, 1H), 8.64 (s, 1H), 7.71 (s, 1H), 3.32-3.24 (m, 2H), 1.60-1.46 (m, 2H), 1.39-1.28 (m, 2H), 0.90 (t, J=7.2 Hz, 3H)

### Step 9: 4-(3-Bromo-6-(butylamino)-1H-pyrazolo[3,4-d]pyrimidin-1-yl)cyclohexanol (14)

To a solution of (4-hydroxycyclohexyl) 4-methylbenzenesulfonate (901 mg, 3.33 mmol) and 3-bromo-N-butyl-1H-pyrazolo[3,4-d]pyrimidin-6-amine (600 mg, 2.22 mmol) in DMF (20 mL) was added Cs₂CO₃ (1.45 g, 4.44 mmol). The reaction mixture was stirred at 110° C. for 16 hours. On completion, the reaction mixture was concentrated in vacuo. The residue was purified by silica gel chromatography (PE: EA=1:1) to give the title compound (500 mg, 49% yield) as a white solid. LC-MS (ESI⁺) m/z 368.0 (M+H)⁺

### Step 10: tert-butyl 4-((4-(6-(butylamino)-1-(4-hydroxycyclohexyl)-1H-pyrazolo[3,4-d]pyrimidin-3-yl)phenyl)sulfonyl)piperazine-1-carboxylate (15)

To a solution of 4-[3-bromo-6-(butylamino)pyrazolo[3,4-d]pyrimidin-1-yl]cyclohexanol (400 mg, 869 μmol), tert-butyl 4-[4-(4,4,5,5-tetramethyl-1,3,2-dioxaborolan-2-yl)phenyl]sulfonylpiperazine-1-carboxylate (472 mg, 1.04 mmol), K₂CO₃ (360 mg, 2.61 mmol) and XPHOS-PD-G₂ (34.2 mg, 43.5 μmol) in water (0.8 mL) and dioxane (4 mL) under N₂. The reaction mixture was stirred at 90° C. for 12 hrs. On completion, the reaction mixture was diluted with ethyl acetate (200 mL) and filtered. The filtrate was concentrated in vacuo. The residue was purified by prep-HPLC (column: Phenomenex Synergi Max-RP 250*50 mm*10 um; mobile phase: [water (0.225% FA)-ACN]; B %: 45ACN %-73ACN %, 30 min, 32% min) to give the title compound 15 (200 mg, 38% yield) as a white solid and 15a (240 mg, 45% yield) as a white solid.

HNMR of 15: ¹H NMR (400 MHz, CDCl₃) δ 8.96 (s, 1H), 8.09 (d, J=8.0 Hz, 2H), 7.86 (d, J=8.0 Hz, 2H), 5.50-5.35 (m, 1H), 4.75-4.60 (m, 1H) 3.92-3.75 (m, 1H), 3.65-3.51 (m, 6H), 3.05-3.02 (m, 4H), 2.25-2.19 (m, 4H), 2.10-2.06 (m, 2H), 1.64-1.60 (m, 4H), 1.58-1.52 (m, 2H), 1.42 (s, 9H), 1.01 (t, J=7.2 Hz, 3H).

HNMR of 15a: ¹H NMR (400 MHz, CDCl₃) δ 8.97 (s, 1H), 8.11 (d, J=8.0 Hz, 2H), 7.86 (d, J=8.0 Hz, 2H), 5.95-5.35 (m, 1H), 4.72-4.66 (m, 1H) 4.17-4.15 (m, 1H), 3.56-3.51 (m, 6H), 3.04-3.03 (m, 4H), 2.57-2.51 (m, 2H), 2.03-1.90 (m, 2H), 1.89-1.82 (m, 4H), 1.51-1.46 (m, 2H), 1.42 (s, 9H), 1.00 (t, J=7.2 Hz, 3H).

### Step 11: (1R,4R)-4-(6-(butylamino)-3-(4-(piperazin-1-ylsulfonyl)phenyl)-1H-pyrazolo[3,4-d]pyrimidin-1-yl)cyclohexanol (17)

To a solution of tert-butyl 4-[4-[(1S)-6-(butylamino)-1-(4-hydroxycyclohexyl)pyrazolo[3,4-d]pyrimidin-3-yl]phenyl]sulfonylpiperazine-1-carboxylate (50.0 mg, 81.5 μmol) in DCM (2 mL) was added HCl/dioxane (4 M, 1 mL). The reaction mixture was stirred at 25° C. for 0.5 hour. On completion, the reaction mixture was concentrated in vacuo to give the title compound (44.8 mg, 100% yield) as a white solid. The crude product was used for the next step without purification. LC-MS (ESI⁺) m/z 514.1 (M+H)⁺

### Step 12: 14-Amino-3,6,9,12-tetraoxatetradecan-1-ol (19)

To a solution of tert-butyl N-[2-[2-[2-[2-(2-hydroxyethoxy)ethoxy]ethoxy]ethoxy]ethyl]carbamate (2.50 g, 7.41 mmol) in DCM (25 mL) was added HCl/dioxane (4 M, 25.0 mL). The reaction mixture was stirred at 25° C. for 15 minutes. On completion, the reaction mixture was concentrated in vacuo to give the title compound (2.00 g, 99% yield, HCl salt) as a yellow oil. ¹H NMR (400 MHz, DMSO-d₆) δ 7.9 (s, 2H), 7.48-7.20 (m, 1H), 3.75-3.25 (m, 20H), 2.90-2.85 (m, 1H)

### Step 13: 2-(2,6-Dioxopiperidin-3-yl)-4-((14-hydroxy-3,6,9,12-tetraoxatetradecyl)amino)isoindoline-1,3-dione (20)

A solution of 2-(2,6-dioxo-3-piperidyl)-4-fluoro-isoindoline-1,3-dione (2.42 g, 8.77 mmol), 2-[2-[2-[2-(2-aminoethoxy)ethoxy]ethoxy]ethoxy]ethanol (2.00 g, 7.31 mmol, HCl salt) and DIPEA (7.55 g, 58.5 mmol, 10.18 mL) in dioxane (30 mL) was stirred at 115° C. for 12 hours. On completion, the reaction mixture was concentrated in vacuo. The residue was purified by column chromatography to give the title compound (2.10 g, 57% yield) as a yellow gum. ¹H NMR (400 MHz, CDCl₃) δ 8.46 (s, 1H), 7.52-7.48 (m, 1H), 7.11 (d, J=7.2 Hz, 1H), 6.93 (d, J=7.2 Hz, 1H), 6.53-6.50 (m, 1H), 4.94-4.89 (m, 1H) 3.74-3.70 (m, 2H), 3.68-3.62 (m, 14H), 3.62-3.58 (m, 2H), 3.48-3.45 (m, 2H), 2.87-2.76 (m, 3H), 2.15-2.12 (m, 1H)

### Step 14: 14-((2-(2,6-Dioxopiperidin-3-yl)-1,3-dioxoisoindolin-4-yl)amino)-3,6,9,12-tetraoxatetra decylmethanesulfonate (21)

To a solution of 2-(2,6-dioxo-3-piperidyl)-4-[2-[2-[2-[2-(2-hydroxyethoxy)ethoxy] ethoxy]ethoxy] ethylamino]isoindoline-1,3-dione (0.20 g, 405 μmol) in DCM (10 mL) was added TEA (123 mg, 1.22 mmol) and MsCl (69.6 mg, 608 μmol). The reaction mixture was stirred at 25° C. for 1 hour. On completion, the reaction mixture was concentrated in vacuo. The residue was purified by silica gel chromatography (PE:EA=1:1) to give the title compound (200 mg, 80% yield) as a yellow oil. ¹H NMR (400 MHz, CDCl₃) δ 8.17 (s, 1H), 7.52-7.49 (m, 1H), 7.12 (d, J=7.2 Hz, 1H), 6.93 (d, J=7.2 Hz, 1H), 6.50 (s, 1H), 4.95-4.92 (m, 1H) 4.39-4.37 (m, 2H), 3.78-3.48 (m, 18H), 3.09 (s, 3H), 2.82-2.77 (m, 3H), 2.14-2.10 (m, 1H).

### Step 15: 4-((14-(4-((4-(6-(Butylamino)-1-(trans-4-hydroxycyclohexyl)-1H-pyrazolo[3,4-d]pyrimidin-3-yl)phenyl)sulfonyl)piperazin-1-yl)-3,6,9,12-tetraoxatetradecyl)amino)-2-(2,6-dioxopiperidin-3-yl)-isoindoline-1,3-dione

To a solution of 4-[(1S)-6-(butylamino)-3-(4-piperazin-1-ylsulfonylphenyl)pyrazolo[3,4-d]pyrimidin-1-yl]cyclohexanol (41.8 mg, 81.5 HCl) and 2-[2-[2-[2-[2-[[2-(2,6-dioxo-3-piperidyl)-1,3-dioxo-isoindolin-4-yl]amino]ethoxy]ethoxy]ethoxy]ethoxy]ethyl methanesulfonate (69.8 mg, 122 μmol) in DMF (2 mL) was added DIPEA (52.6 mg, 407 μmol). The reaction mixture was stirred at 100° C. for 18 hours. On completion, the reaction mixture was concentrated in vacuo. The residue was purified by prep-HPLC (column: Phenomenex Gemini 150*25 mm*10 um; mobile phase: [water (0.05% ammonia hydroxide v/v)-ACN]; B %: 43%-73%) to give the title compound (14.5 mg, 17% yield) as a yellow solid. ¹H NMR (400 MHz, DMSO-d₆) δ 11.09 (s, 1H), 9.17 (s, 1H), 8.22 (d, J=8.4, Hz, 2H), 7.81 (d, J=8.4, Hz, 2H), 7.65 (s, 1H), 7.58-7.54 (m, 1H), 7.11 (d, J=8.4 Hz, 1H), 7.02 (d, J=8.4 Hz, 1H), 6.59-6.01 (m, 1H), 5.06-5.01 (m, 1H), 4.74-4.73 (m, 1H), 4.55-4.45 (m, 1H), 3.59-3.41 (m, 22H), 2.95-2.85 (m, 4H), 2.85-2.80 (m, 1H), 2.60-2.55 (m, 1H), 2.53-2.50 (m, 1H), 2.50-2.45 (m, 4H), 2.45-2.42 (m, 2H), 2.08-2.00 (m, 1H), 1.99-1.80 (m, 6H), 1.60-1.53 (m, 2H), 1.40-1.36 (m, 4H), 0.90-0.91 (m, 3H). LC-MS (ESI⁺) m/z 989.5 (M+H)⁺

### EXAMPLE 23: 4-((14-(4-((4-(6-(butylamino)-1-(trans-4-hydroxycyclohexyl)-1H-pyrazolo[3,4-d]pyrimidin-3-yl)phenyl)sulfonyl)piperazin-1-yl)-14-oxo-3,6,9,12-tetraoxatetradecyl)amino)-2-(2,6-dioxopiperidin-3-yl)isoindoline-1,3-dione (I-32)

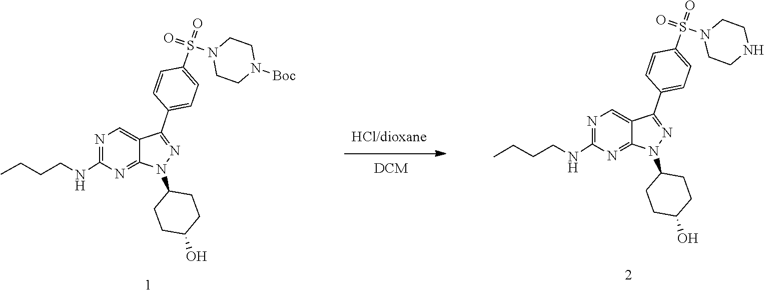 [SMILES: CCCCNc1ncc2c(-c3ccc(S(=O)(=O)N4CCN(C(=O)OC(C)(C)C)CC4)cc3)nn([C@H]3CC[C@H](O)CC3)c2n1 ; CCCCNc1ncc2c(-c3ccc(S(=O)(=O)N4CCNCC4)cc3)nn([C@H]3CC[C@H](O)CC3)c2n1]

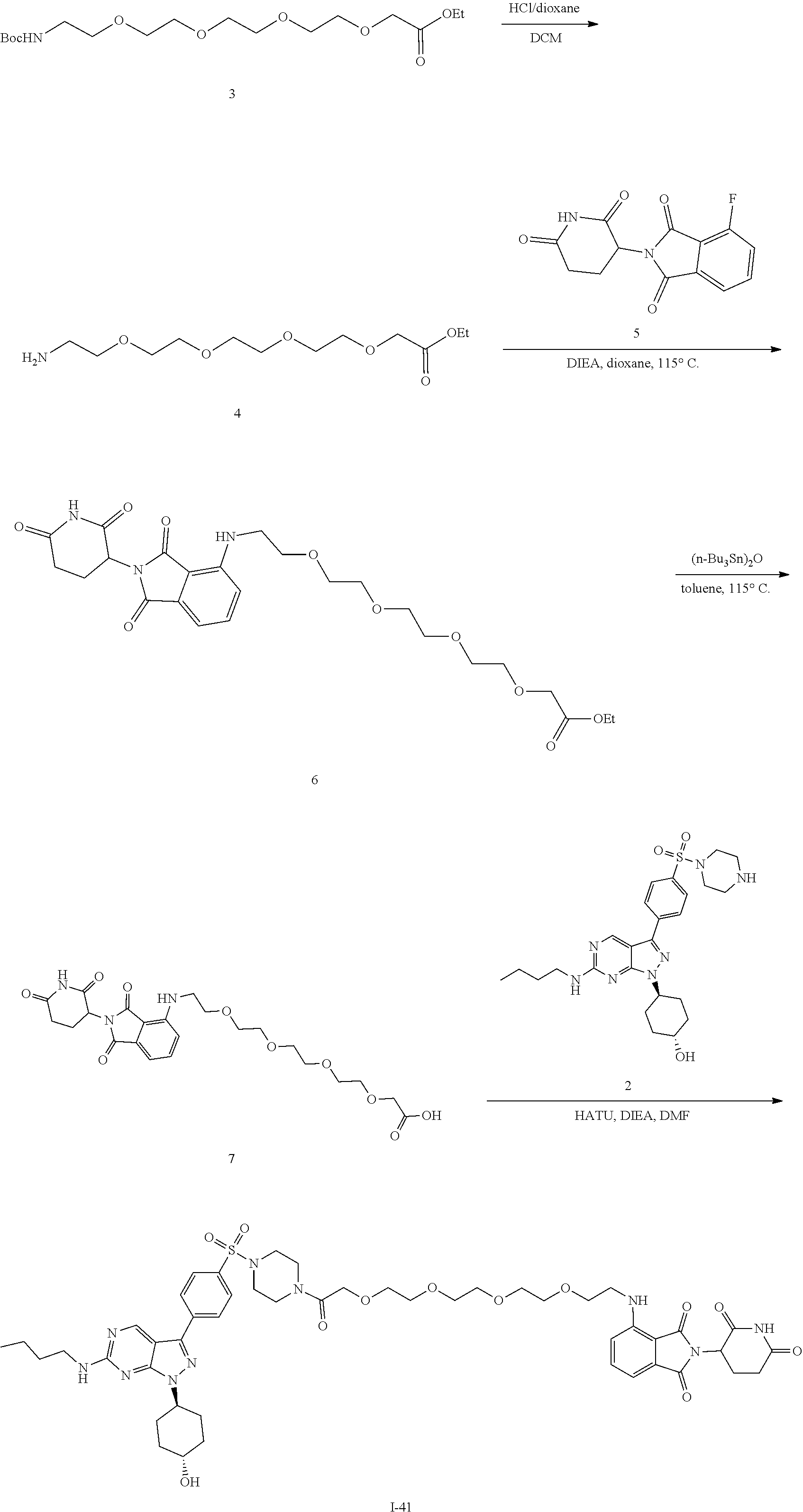

### Step 1: 4-[6-(Butylamino)-3-(4-piperazin-1-ylsulfonylphenyl)pyrazolo[3,4-d]pyrimidin-1-yl]cyclohexanol (2)

To a solution of tert-butyl 4-[4-[6-(butylamino)-1-(4-hydroxycyclohexyl)pyrazolo[3,4-d]pyrimidin-3-yl]phenyl]sulfonylpiperazine-1-carboxylate (0.08 g, 130 μmol) in DCM (10 mL) was added HCl/dioxane (4 M, 2.00 mL). The reaction mixture was stirred at 25° C. for 0.5 hour. On completion, the reaction mixture was concentrated in vacuo to give the title compound (0.07 g, 98% yield, HCl) as off-white solid. The crude was used to next step directly without further purification. LC-MS (ESI⁺) m/z 514.1 (M+H)⁺

### Step 2: Ethyl 2-[2-[2-[2-(2-aminoethoxy)ethoxy]ethoxy]ethoxy]acetate (4)

To a solution of ethyl 2-[2-[2-[2-[2-(tert-butoxycarbonylamino)ethoxy]ethoxy]ethoxy]ethoxy]acetate (0.80 g, 2.11 mmol) in DCM (10 mL) was added HCl/dioxane (4 M, 2.67 mL). The reaction mixture was stirred at 25° C. for 15 minutes. On completion, the reaction mixture was concentrated in vacuo to give the title compound (0.55 g, 83% yield, HCl) as a yellow oil. The crude was used for next step directly without further purification.

### Step 3: Ethyl 2-[2-[2-[2-[2-[[2-(2,6-dioxo-3-piperidyl)-1,3-dioxo-isoindolin-4-yl]amino]ethoxy]ethoxy]ethoxy]ethoxy]acetate (6)

To a solution of ethyl 2-[2-[2-[2-(2-aminoethoxy)ethoxy]ethoxy]ethoxy]acetate (0.55 g, 1.74 mmol, HCl), 2-(2,6-dioxo-3-piperidyl)-4-fluoro-isoindoline-1,3-dione (577 mg, 2.09 mmol) in dioxane (20 mL) was added diisopropylethylamine (1.80 g, 13.9 mmol). Then the reaction mixture was stirred at 115° C. for 16 hours. On completion, the reaction mixture was concentrated in vacuo. The residue was purified by column chromatography (petroleum ether:ethyl acetate=1:1) to give the title compound (0.25 g, 27% yield) as a yellow gum.

¹H NMR (400 MHz, CDCl₃) δ=8.21 (br s, 1H), 7.53-7.47 (m, 1H), 7.11 (d, J=7.2 Hz, 1H), 6.93 (d, J=8.4 Hz, 1H), 6.54-6.47 (m, 1H), 4.95-4.89 (m, 1H), 4.24-4.19 (m, 2H), 4.16-4.15 (m, 2H), 3.75-3.68 (m, 14H), 3.48 (q, J=5.6 Hz, 2H), 2.94-2.70 (m, 3H), 2.17-2.09 (m, 1H), 1.32 (t, J=5.6 Hz, 2H).

### Step 4: 2-[2-[2-[2-[2-[[2-(2,6-Dioxo-3-piperidyl)-1,3-dioxo-isoindolin-4-yl]amino]ethoxy]ethoxy]ethoxy]ethoxy]acetic acid (7)

To a solution of (Bu₃Sn)₂O (779 mg, 1.31 mmol) in toluene (20 mL) was added ethyl 2-[2-[2-[2-[2-[[2-(2,6-dioxo-3-piperidyl)-1,3-dioxo-isoindolin-4-yl]amino]ethoxy]ethoxy] ethoxy]ethoxy]acetate (0.35 g, 654 μmol). The reaction mixture was stirred at 115° C. for 12 hours. On completion, the reaction mixture was quenched with 1 N sodium fluoride solution. The crude was purified by reverse phase flash (0.1% FA) to give the title compound (0.175 g, 53% yield) as a yellow oil. LC-MS (ESI⁺) m/z 508.2 (M+H)⁺

### Step 5: 2-[2-[2-[2-[2-[4-[4-[6-(Butylamino)-1-(trans-4-hydroxycyclohexyl)pyrazolo[3,4-d]pyrimidin-3-yl]phenyl]sulfonylpiperazin-1-yl]-2-oxo-ethoxy]ethoxy]ethoxy]ethoxy]ethylamino]-2-(2,6-dioxo-3-piperidyl)isoindoline-1,3-dione

To a solution of 4-[6-(butylamino)-3-(4-piperazin-1-ylsulfonylphenyl)pyrazolo[3,4-d]pyrimidin-1-yl]cyclohexanol (91.1 mg, 166 HCl) and DIEA (89.1 mg, 690 μmol) in DMF (10 mL) was added 2-[2-[2-[2-[2-[[2-(2,6-dioxo-3-piperidyl)-1,3-dioxo-isoindolin-4-yl]amino]ethoxy]ethoxy] ethoxy]ethoxy]acetic acid (0.07 g, 138 μmol) and HATU (68.2 mg, 179 μmol). The reaction mixture was stirred at 25° C. for 0.5 hour. On completion, the reaction mixture was concentrated in vacuo. The residue was purified by prep-HPLC (column: Phenomenex Synergi C18 150*25*10 um; mobile phase: [water (0.225% FA)-ACN]) to give the title compound (44.0 mg, 32% yield) as a yellow solid. ¹H NMR (400 MHz, DMSO-d₆) δ=11.10 (s, 1H), 9.17 (br s, 1H), 8.22 (d, J=8.4 Hz, 2H), 7.82 (d, J=8.4 Hz, 2H), 7.72-7.61 (m, 1H), 7.56 (dd, J=7.2, 8.4 Hz, 1H), 7.12 (d, J=8.4 Hz, 1H), 7.03 (d, J=7.2 Hz, 1H), 6.62-6.54 (m, 1H), 5.04 (dd, J=5.6, 12.8 Hz, 1H), 4.60-4.46 (m, 1H), 4.06 (s, 2H), 3.67-3.43 (m, 21H), 3.04-2.91 (m, 4H), 2.90-2.80 (m, 1H), 2.64-2.53 (m, 2H), 2.16-1.85 (m, 8H), 1.65-1.50 (m, 2H), 1.47-1.30 (m, 4H), 0.93 (t, J 4.0, 3H). LC-MS (ESI⁺) m/z 1025.4 (M+Na)⁺

### EXAMPLE 24: (2S,4R)-1-((S)-2-(tert-butyl)-17-(4-((4-(6-(butylamino)-1-(trans-4-hydroxycyclohexyl)-1H-pyrazolo[3,4-d]pyrimidin-3-yl)phenyl)sulfonyl)piperazin-1-yl)-4-oxo-6,9,12,15-tetraoxa-3-azaheptadecanoyl)-4-hydroxy-N-(4-(4-methylthiazol-5-yl)benzyl)pyrrolidine-2-carboxamide (I-33)

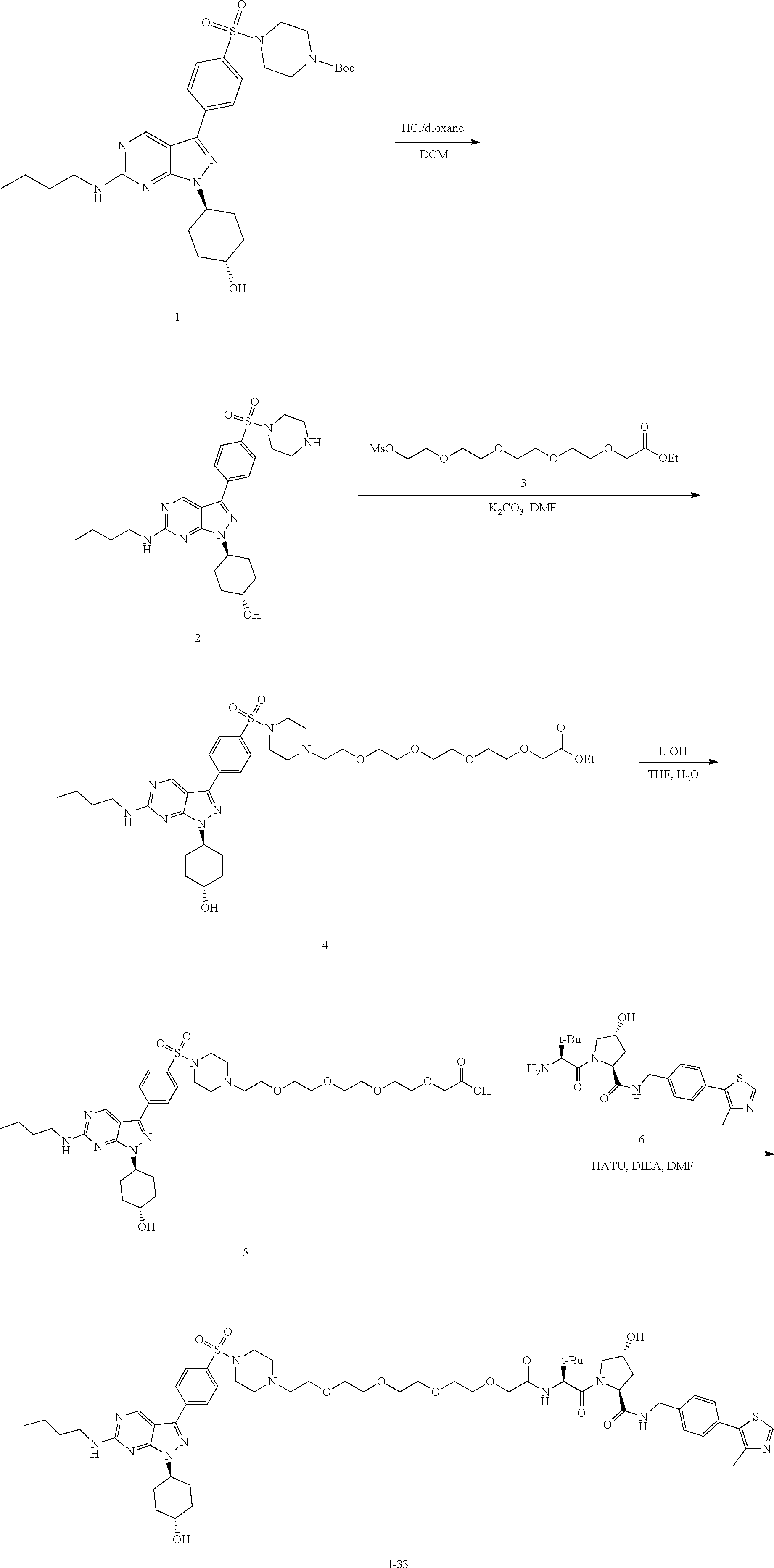

### Step 1: 4-[(1S)-6-(butylamino)-3-(4-piperazin-1-ylsulfonylphenyl)pyrazolo[3,4-d]pyrimidin-1-yl]cyclohexanol (2)

To a solution of tert-butyl-4-[4-[(1S)-6-(butylamino)-1-(4-hydroxycyclohexyl) pyrazolo[3,4-d] pyrimidin-3-yl]phenyl]sulfonylpiperazine-1-carboxylate (100 mg, 163 μmol) in DCM (2 mL) was added HCl/dioxane (4 M, 2 mL). The reaction mixture was stirred at 25° C. for 10 minutes. On completion, the reaction mixture was concentrated in vacuo to give the title compound (80.0 mg, 89% yield, HCl) as a yellow solid. The residue was used for the next step directly without further purification.

LC-MS (ESI⁺) m/z 514.2 (M+H)⁺

### Step 2: Ethyl 2-[2-[2-[2-[2-[4-[4-[(1S)-6-(butylamino)-1-(4-hydroxycyclohexyl)pyrazolo[3,4-d]pyrimidin-3-yl]phenyl]sulfonylpiperazin-1-yl]ethoxy]ethoxy]ethoxy]ethoxy]acetate (4)

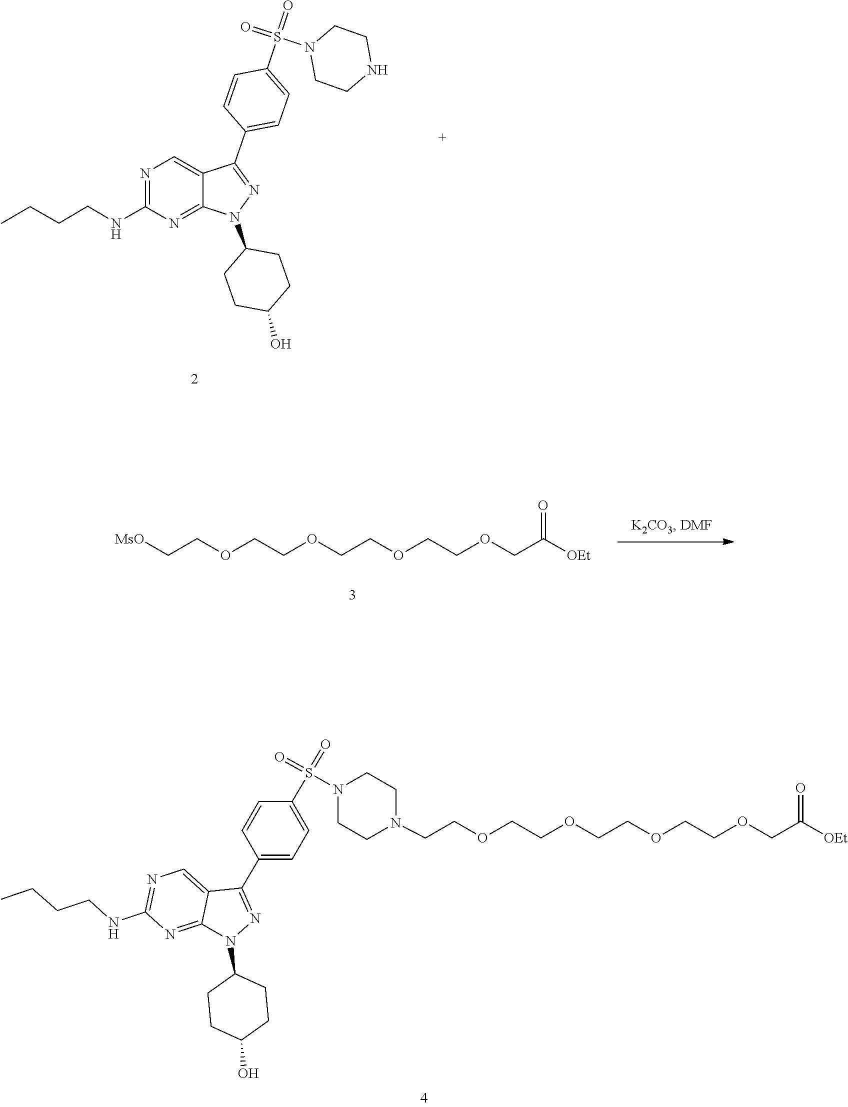 [SMILES: CCCCNc1ncc2c(-c3ccc(S(=O)(=O)N4CCNCC4)cc3)nn([C@H]3CC[C@H](O)CC3)c2n1 ; CCOC(=O)COCCOCCOCCOCCOS(C)(=O)=O ; CCCCNc1ncc2c(-c3ccc(S(=O)(=O)N4CCN(CCOCCOCCOCCOCC(=O)OCC)CC4)cc3)nn([C@H]3CC[C@H](O)CC3)c2n1]

To a solution of 4-[(1S)-6-(butylamino)-3-(4-piperazin-1-ylsulfonylphenyl) pyrazolo[3,4-d]pyrimidin -1-yl]cyclohexanol (80.0 mg, 145 μmol, HCl) and ethyl 2-[2-[2-[2-(2-methylsulfonyloxyethoxy) ethoxy]ethoxy]ethoxy]acetate (130 mg, 364 μmol) in DMF (2.5 mL) was added K₂CO₃ (60.3 mg, 436 μmol). The reaction mixture was stirred at 90° C. for 20 hours. On completion, the mixture was filtered and the filtrate was acidified with FA till pH=3-5. The above mixture was purified by prep-HPLC (column: Phenomenex Synergi C18 150*25*10 um; mobile phase: [water (0.225% FA)-ACN]; B %: 25%-46%, 10 min) to give the title compound (22.0 mg, 20% yield) as a yellow solid. LC-MS (ESI⁺) m/z 776.4 (M+H)⁺

### Step 3: 2-[2-[2-[2-[2-[4-[4-[6-(Butylamino)-1-(4-hydroxycyclohexyl)pyrazolo[3,4-d]pyrimidin-3-yl]phenyl]sulfonylpiperazin-1-yl]ethoxy]ethoxy]ethoxy]ethoxy]acetic acid (5)

To a solution of ethyl 2-[2-[2-[2-[2-[4-[4-[6-(butylamino)-1-(4-hydroxycyclohexyl)pyrazolo[3,4-d]pyrimidin-3-yl]phenyl]sulfonylpiperazin-1-yl]ethoxy]ethoxy]ethoxy]ethoxy]acetate (22.0 mg, 28.4 μmol) in a mixed solvent of THF (1 mL) and H₂O (0.2 mL) was added LiOH (3.39 mg, 142 μmol). The reaction mixture was stirred at 25° C. for 0.5 hour. On completion, the mixture was acidified with 12N HCl solution till pH=3, then concentrated in vacuo to give the title compound (21.0 mg, 99% yield) as a yellow solid. The residue was used for the next step directly without further purification. LC-MS (ESI⁺) m/z 748.1 (M+H)⁺

### Step 4: (2S,4R)-1-[(2S)-2-[[2-[2-[2-[2-[2-[4-[4-[6-(butylamino)-1-(4-hydroxycyclohexyl)pyrazolo[3,4-d]pyrimidin-3-yl]phenyl]sulfonylpiperazin-1-yl]ethoxy]ethoxy]ethoxy]ethoxy]acetyl]amino]-3,3-dimethyl-butanoyl]-4-hydroxy-N-[[4-(4-methylthiazol-5-yl)phenyl]methyl]pyrrolidine-2-carboxamide

To a solution of 2-[2-[2-[2-[2-[4-[4-[6-(butylamino)-1-(4-hydroxycyclohexyl) pyrazolo[3,4-d]pyrimidin-3-yl]phenyl]sulfonylpiperazin-1-yl]ethoxy]ethoxy]ethoxy]ethoxy] acetic acid (21.0 mg, 28.1 μmol) and (2S,4R)-1-[(2S)-2-amino-3,3-dimethyl-butanoyl]-4-hydroxy-N-[[4-(4-methylthiazol-5-yl) phenyl]methyl]pyrrolidine-2-carboxamide (12.1 mg, 28.1 μmol) in DMF (1 mL) was added DIEA (18.1 mg, 140 μmol). Ten minutes later, HATU (12.8 mg, 33.7 μmol) was added into the mixture. The reaction mixture was stirred at 25° C. for 30 minutes. On completion, the mixture was filtered and the filtrate was acidified with FA till pH=5-7. The mixture was purified by prep-HPLC (column: Phenomenex Synergi C18 150*25*10 um; mobile phase: [water (0.225% FA)-ACN]; B %: 30%-48%, 10 min) to give the title compound (13.7 mg, 39% yield) as a yellow solid. ¹H NMR (400 MHz, DMSO-d₆) δ=9.18 (s, 1H), 8.97 (s, 1H), 8.58 (t, J=5.6 Hz, 1H), 8.22 (d, J=8.4 Hz, 2H), 7.82 (d, J=8.4 Hz, 2H), 7.65 (s, 1H), 7.40-7.37 (m, 1H), 7.40-7.35(m, 4H), 5.16 (br s, 1H), 4.72 (br s, 1H), 4.56 (br d, J=9.6 Hz, 2H), 4.47-4.43 (m, 1H), 4.42-4.37 (m, 1H), 4.35-4.32 (m, 1H), 4.27-4.19 (m, 1H), 3.95 (s, 2H), 3.69-3.43 (m, 19H), 3.00-2.82 (m, 4H), 2.54-2.52 (m, 4H), 2.46-2.43 (m, 2H), 2.43 (s, 3H), 2.09-1.86 (m, 8H), 1.66-1.62 (m, 2H), 1.43-1.32 (m, 4H), 0.97-0.89 (m, 12H). LC-MS (ESI⁺) m/z 1160.6 (M+H)⁺

### EXAMPLE 25: (2S,4R)-1-((S)-2-(tert-butyl)-17-(4-((4-(6-(butylamino)-1-(trans-4-hydroxycyclohexyl)-1H-pyrazolo[3,4-d]pyrimidin-3-yl)phenyl)sulfonyl)piperazin-1-yl)-4,17-dioxo-6,9,12,15-tetraoxa-3-azaheptadecanoyl)-4-hydroxy-N-(4-(4-methylthiazol-5-yl)benzyl)pyrrolidine-2-carboxamide (I-31)

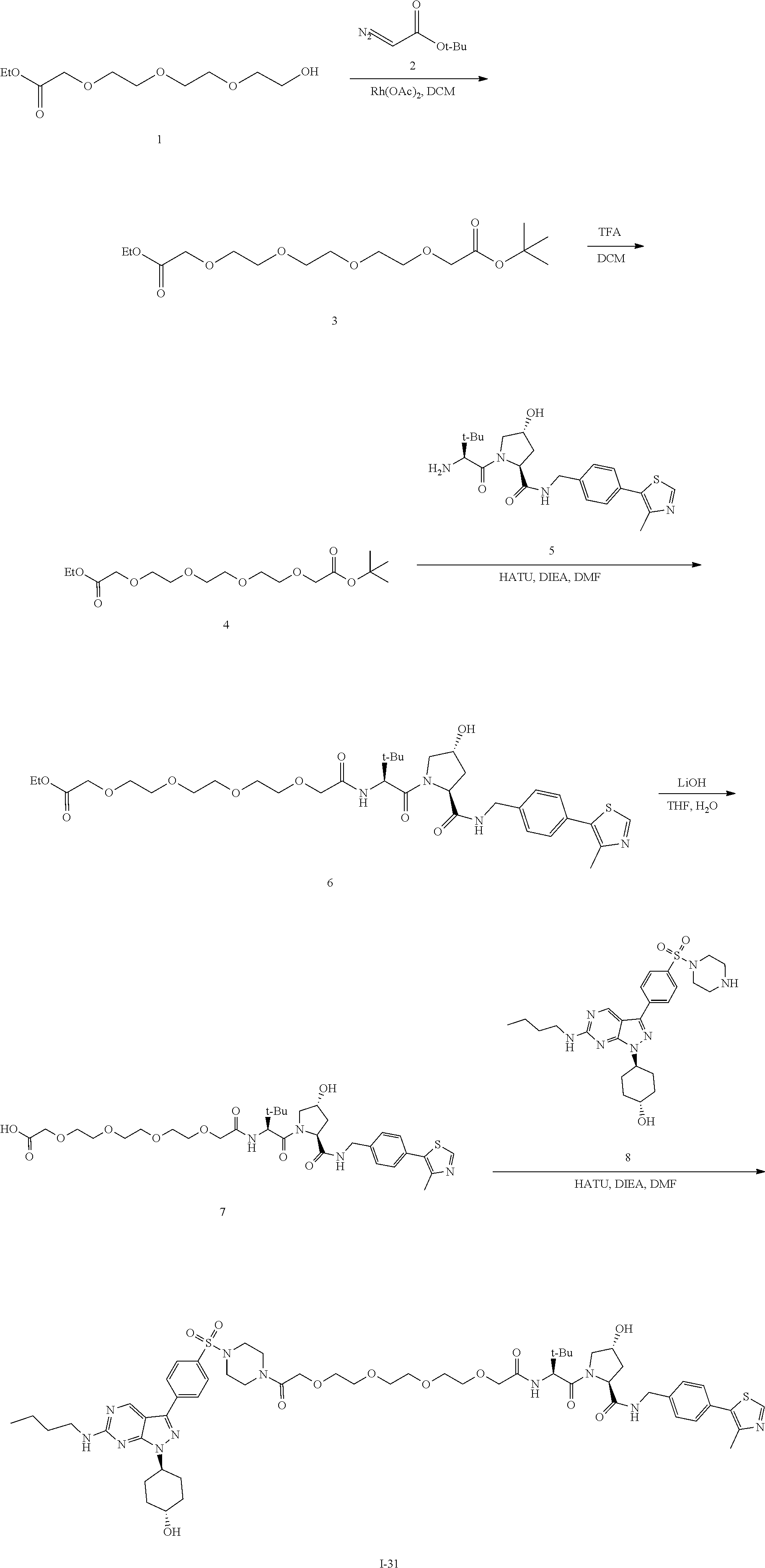

### Step 1: Ethyl 2-[2-[2-[2-(2-tert-butoxy-2-oxo-ethoxy)ethoxy]ethoxy]ethoxy]acetate (3)

To a solution of ethyl 2-[2-[2-(2-hydroxyethoxy)ethoxy]ethoxy]acetate (1.00 g, 4.23 mmol) and Rh(OAc)₂ (37.4 mg, 169 μmol) in DCM (20 mL) was added a solution of tert-butyl 2-diazoacetate (602 mg, 4.23 mmol) in DCM (40 mL). The reaction mixture was stirred at 25° C. for 16 hours. On completion, the mixture was diluted with H₂O (100 mL), extracted with DCM (2×100 mL). The combined organic phase was dried over Na₂SO₄, filtered and concentrated in vacuo to give a residue. The residue was purified by column chromatography to give the title compound (0.80 g, 54% yield) as a yellow oil. ¹H NMR (400 MHz, CDCl₃) δ 4.15 (q, J=7.2 Hz, 2H), 4.08 (s, 2H), 3.95 (s, 2H), 3.70-3.57 (m, 12H), 1.41 (s, 9H), 1.22 (t, J=7.2 Hz, 3H)

### Step 2: 2-[2-[2-[2-(2-Ethoxy-2-oxo-ethoxy)ethoxy]ethoxy]ethoxy]acetic acid (4)

To a solution of ethyl 2-[2-[2-[2-(2-tert-butoxy-2-oxo-ethoxy)ethoxy] ethoxy]ethoxy]acetate (340 mg, 970 μmol) in DCM (5 mL) was added TFA (1.11 g, 9.70 mmol, 718 μL). The reaction mixture was stirred at 25° C. for 30 minutes. On completion, the reaction mixture was concentrated in vacuo to give the title compound (250 mg, 87% yield) as a yellow oil. The residue was used to the next step directly without further purification.

### Step 3: Ethyl 2-[2-[2-[2-[2-[[(1S)-1-[(2S,4R)-4-hydroxy-2-[[4-(4-methylthiazol-5-yl)pheny]methyl carbamoyl]pyrrolidine-1-carbonyl]-2,2-dimethyl-propyl]amino]-2-oxo-ethoxy]ethoxy]ethoxy]ethoxy]acetate (6)

To a solution of 2-[2-[2-[2-(2-ethoxy-2-oxo-ethoxy)ethoxy]ethoxy]ethoxy]acetic acid (200 mg, 679 μmol) and (2S,4R)-1-[(2S)-2-amino-3,3-dimethyl-butanoyl]-4-hydroxy-N-[[4-(4-methylthiazol-5-yl)phenyl] methyl]pyrrolidine-2-carboxamide (317 mg, 679 μmol) in DMF (5 mL) was added DIEA (527 mg, 4.08 mmol, 710 μL), the mixture was stirred at 25° C. for 12 min. Then HATU (388 mg, 1.02 mmol) was added to the mixture, the reaction mixture was stirred at 25° C. for another 1 hour. On completion, the mixture was quenched by addition H₂O (20 mL) and extracted with EA (2×40 mL). The combined organic layers were dried over Na₂SO₄, filtered and concentrated in vacuo to give a residue. The residue was purified by reverse phase (0.5% NH₃.H₂O condition) to give the title compound (340 mg, 63% yield) as a yellow oil. LC-MS (ESI⁺) m/z 707.4 (M+H)⁺

### Step 4: 2-[2-[2-[2-[2-[[(1S)-1-[(2S,4R)-4-hydroxy-2-[[4-(4-methylthiazol-5-yl)pheny]methyl carbamoyl]pyrrolidine-1-carbonyl]-2,2-dimethyl-propyl]amino]-2-oxo-ethoxy]ethoxy]ethoxy]ethoxy]acetic acid (7)

To a solution of ethyl 2-[2-[2-[2-[2-[[(1S)-1-[(2S,4R)-4-hydroxy-2-[[4-(4-methylthiazol-5-yl)phenyl]methyl carbamoyl]pyrrolidine-1-carbonyl]-2,2-dimethyl-propyl] amino]-2-oxo-ethoxy]ethoxy]ethoxy]ethoxy]acetate (100 mg, 141 μmol) in THF (3 mL) and H₂O (1 mL) was added LiOH (5.08 mg, 212 μmol). The reaction mixture was stirred at 25° C. for 1.5 hours. On completion, the mixture was concentrated in vacuo to remove THF, then diluted with H₂O (5 mL). The aqueous phase was acidified with conc.HCl till pH=5, the mixture was concentrated in vacuo to give the title compound (96.0 mg, 93% yield) as a white solid. LC-MS (ESI⁺) m/z 679.1 (M+H)⁺

### Step 5: (2S,4R)-1-[(2S)-2-[[2-[2-[2-[2-[2-[4-[4-[6-(butylamino)-1-(trans-4-hydroxycyclohexyl)pyrazolo[3,4-d]pyrimidin-3-yl]phenyl]sulfonylpiperazin-1-yl]-2-oxo-ethoxy]ethoxy]ethoxy]ethoxy]acetyl]amino]-3,3-dimethyl-butanoyl]-4-hydroxy-N-[[4-(4-methylthiazol-5-yl)phenyl]methyl]pyrrolidine-2-carboxamide

To a solution of 2-[2-[2-[2-[2-[[(1S)-1-[(2S,4R)-4-hydroxy-2-[[4-(4-methylthiazol-5-yl)phenyl] methylcarbamoyl]pyrrolidine-1-carbonyl]-2,2-dimethyl-propyl]amino]-2-oxo-ethoxy]ethoxy]ethoxy]ethoxy]acetic acid (56.0 mg, 82.5 μmol) and 4-[6-(butylamino)-3-(4-piperazin-1-ylsulfonylphenyl) pyrazolo [3,4-d] pyrimidin-1-yl]cyclohexanol (42.3 mg, 82.5 μmol) in DMF (2 mL) was added DIEA (53.3 mg, 412 μmol, 71.8 μL). The mixture was stirred at 25° C. for 12 minutes. Then HATU (31.4 mg, 82.5 μmol) was added to the mixture, the reaction mixture was stirred at 25° C. for 2 hours. On completion, the reaction mixture was concentrated in vacuo to give a residue. The residue was purified by prep-HPLC (column: Boston Green ODS 150*30 5 u; mobile phase: [water (0.225% FA)-ACN]; B %: 47%-71%, 10 min) to give the title compound (46.5 mg, 46% yield) as a yellow solid. ¹H NMR (400 MHz, DMSO-d₆) δ 9.17 (s, 1H), 8.97 (s, 1H), 8.58 (t, J=6.0 Hz, 1H), 8.23 (d, J=8.4 Hz, 2H), 7.82 (d, J=8.4 Hz, 2H), 7.65 (s, 1H), 7.39 (m, 5H), 5.17 (s, 1H), 5.29-4.94 (m, 1H), 4.54-4.48 (m, 1H), 4.46-4.37 (m, 2H), 4.36-4.32 (m, 1H), 4.28-4.21 (m, 1H), 4.07-4.04 (m, 2H), 3.95 (s, 2H), 3.63-3.44 (m, 18H), 2.95-2.93 (m, 4H), 2.53-2.50 (m, 4H), 2.43-2.40 (m, 3H), 2.08-1.88 (m, 8H), 1.58-1.55 (m, 2H), 1.39-1.36 (m, 4H), 0.93 (s, 12H). LC-MS (ESI⁺) m/z 1196.6 (M+Na)⁺

### EXAMPLE 26: 4-((2-(2-(2-(4-((4-(6-(butylamino)-1-(trans-4-hydroxycyclohexyl)-1H-pyrazolo[3,4-d]pyrimidin-3-yl)phenyl)sulfonyl)piperazin-1-yl)ethoxy)ethoxy)ethyl)amino)-2-(2,6-dioxopiperidin-3-yl)isoindoline-1,3-dione (I-30)

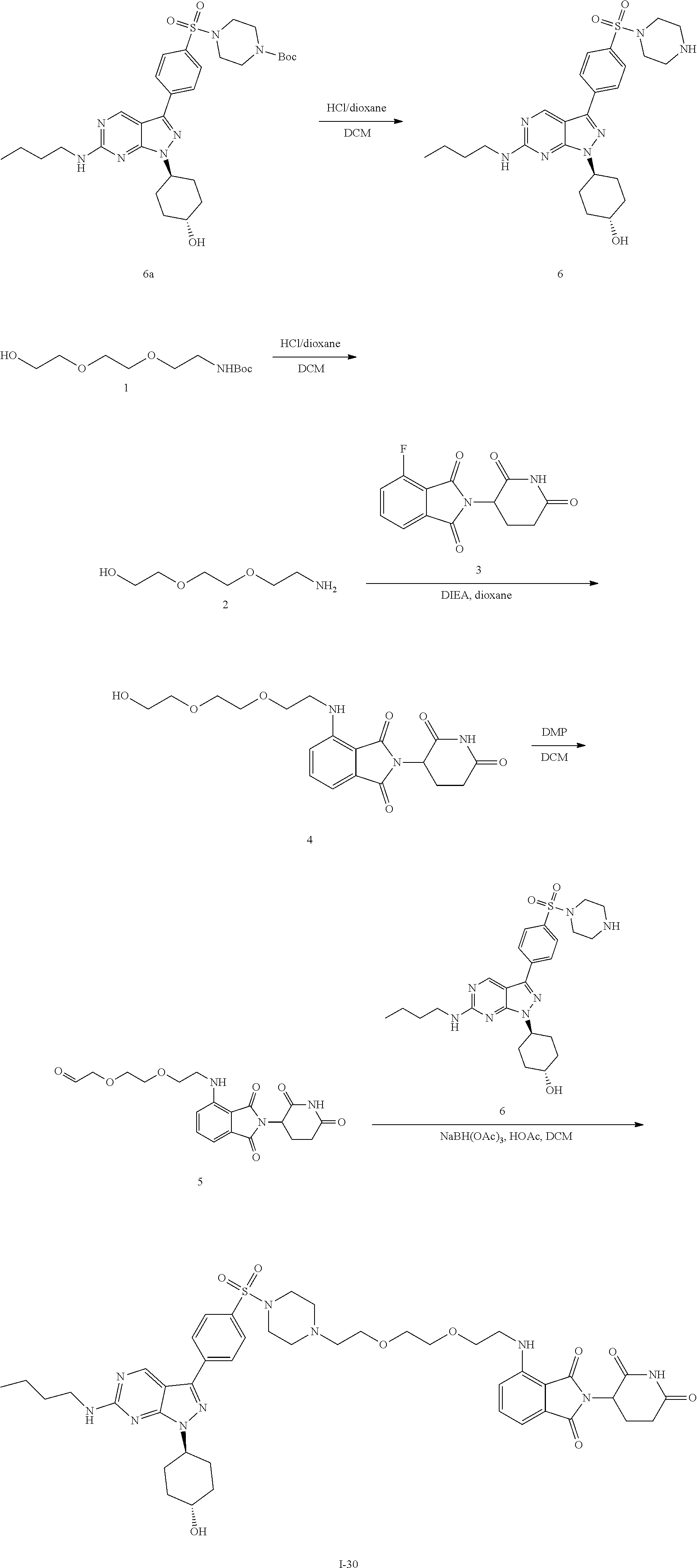

### Step 1: 4-[6-(Butylamino)-3-(4-piperazin-1-ylsulfonylphenyl)pyrazolo[3,4-d]pyrimidin-1-yl]cyclohexanol (6)

To a mixture of tert-butyl 4-[4-[6-(butylamino)-1-(4-hydroxycyclohexyl)pyrazolo [3,4-d]pyrimidin-3-yl]phenyl]sulfonylpiperazine-1-carboxylate (60.0 mg, 97.7 μmol) in DCM (3 mL) was added HCl/dioxane (4 M, 1 mL). The reaction mixture was stirred at 25° C. for 0.5 hour. On completion, the reaction mixture was concentrated in vacuo to give the title compound (55.0 mg, 95% yield, HCl) as a white solid. LC-MS (ESI⁺) m/z 514.3 (M+H)⁺

### Step 2: 2-[2-(2-Aminoethoxy)ethoxy]ethanol (2)

To a solution of tert-butyl N-[2-[2-(2-hydroxyethoxy)ethoxy]ethyl]carbamate (3.00 g, 12.0 mmol) in DCM (30 mL) was added HCl/dioxane (4 M, 15 mL). The reaction mixture was stirred at 25° C. for 20 minutes. On completion, the residue was concentrated in vacuo to give the compound (1.80 g, 100% yield) as yellow oil. The crude product was used to the next step directly without further purification.

### Step 3: 2-(2,6-Dioxo-3-piperidyl)-4-[2-[2-(2-hydroxyethoxy)ethox]ethylamino]isoindoline-1,3-dione (4)

To a solution of 2-[2-(2-aminoethoxy)ethoxy]ethanol (1.80 g, 12.0 mmol) and 2-(2,6-dioxo-3-piperidyl)-4-fluoroisoindoline-1,3-dione (4.00 g, 14.4 mmol, from IRW-003) in dioxane (35 mL) was added DIEA (9.36 g, 72.3 mmol). The reaction mixture was stirred at 115° C. for 20 hours. On completion, the mixture was concentrated in vacuo. The residue was purified by silica gel chromatography to give the title compound (2.90 g, 59% yield) as a yellow solid. ¹H NMR (400 MHz, CDCl₃) δ 8.22 (s, 1H), 7.42 (dd, J=7.6, 8.4 Hz, 1H), 7.03 (d, J=7.2 Hz, 1H), 6.84 (d, J=8.4 Hz, 1H), 6.50 (t, J=5.6 Hz, 1H), 4.85 (dd, J=5.6, 11.6 Hz, 1H), 3.69-3.64 (m, 4H), 3.62 (s, 4H), 3.57-3.51 (m, 2H), 3.41 (q, J=5.6 Hz, 2H), 2.84-2.64 (m, 3H), 2.10-2.00 (m, 1H), 1.65 (s, 1H)

### Step 4: 2-[2-[2-[[2-(2,6-Dioxo-3-piperidyl)-1,3-dioxo-isoindolin-4-yl]amino]ethoxy]ethoxy]acetaldehyde (5)

To a mixture of 2-(2,6-dioxo-3-piperidyl)-4-[2-[2-(2-hydroxyethoxy)ethoxy] ethylamino]isoindoline-1,3-dione (100 mg, 246 μmol) in DCM (4 mL) was added DMP (209 mg, 493 μmol) at 0° C. The reaction mixture was stirred at 25° C. for 12 hours. On completion, the reaction mixture was diluted with Na₂S₂O₃ (10 mL), NaHCO₃ (10 mL) and stirred for 30 min. then the mixture was extracted with DCM (3×10 mL). The combined organic layers was dried over Na₂SO₄, filtered and concentrated in vacuo to give the title compound (92.0 mg, 92% yield) as a yellow solid. LC-MS (ESI⁺) m/z 404.0 (M+H)⁺

### Step 5: 4-[2-[2-[2-[4-[4-[6-(Butylamino)-1-(trans-4-hydroxycyclohexyl)pyrazolo[3,4-d]pyrimidin-3-yl]phenyl]sulfonylpiperazin-1-yl]ethoxy]ethoxy]ethylamino]-2-(2,6-dioxo-3-piperidyl)isoindoline-1,3-dione

To a mixture of 4-[6-(butylamino)-3-(4-piperazin-1-ylsulfonylphenyl)pyrazolo[3,4-d]pyrimidin-1-yl] cyclohexanol (25.2 mg, 45.8 HCl) and 2-[2-[2-[[2-(2,6-dioxo-3-piperidyl)-1,3-dioxo-isoindolin -4-yl]amino]ethoxy]ethoxy]acetaldehyde (18.0 mg, 44.6 μmol) in a mixed solvent of DCM (2 mL) and THF (3 mL) was added HOAc (3.48 mg, 58.0 μmol) and NaBH(OAc)₃ (13.2 mg, 62.4 μmol). The reaction mixture was stirred at 25° C. for 16 hours. On completion, the reaction mixture was concentrated in vacuo. The residue was purified by prep-HPLC (column: Phenomenex Synergi C18 150*25*10 um; mobile phase: [water (0.225% FA)-ACN]) to give the title compound (19.8 mg, 47% yield) as yellow solid. ¹H NMR (400 MHz, DMSO-d₆) δ 11.10 (s, 1H), 9.17 (s, 1H), 8.22 (d, J=8.4 Hz, 2H), 7.80 (d, J=8.4 Hz, 2H), 7.65 (s, 1H), 7.55 (dd, J=7.2, 8.4 Hz, 1H), 7.10 (d, J=8.8 Hz, 1H), 7.02 (d, J=6.7 Hz, 1H), 6.56 (t, J=5.6 Hz, 1H), 5.05 (dd, J=5.6, 12.8 Hz, 1H), 4.98-4.53 (m, 1H), 4.53-4.49 (m, 1H), 3.59-3.56 (m, 2H), 3.56-3.53 (m, 1H), 3.52-3.43 (m, 10H), 2.94-2.86 (m, 4H), 2.85-2.81 (m, 1H), 2.63-2.56 (m, 1H), 2.55-2.54 (m, 1H), 2.53 (d, J=2.0 Hz, 4H), 2.44-2.42 (m, 2H), 2.15-2.08 (m, 1H), 2.05-1.89 (m, 6H), 1.65-1.54 (m, 2H), 1.44-1.32 (m, 4H), 0.94 (s, 3H) LC-MS (ESI⁺) m/z 901.3 (M+H)⁺

### EXAMPLE 27: Trans-3-[4-[15-(4-[[4-(2-[[(2R)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid) (I-5)

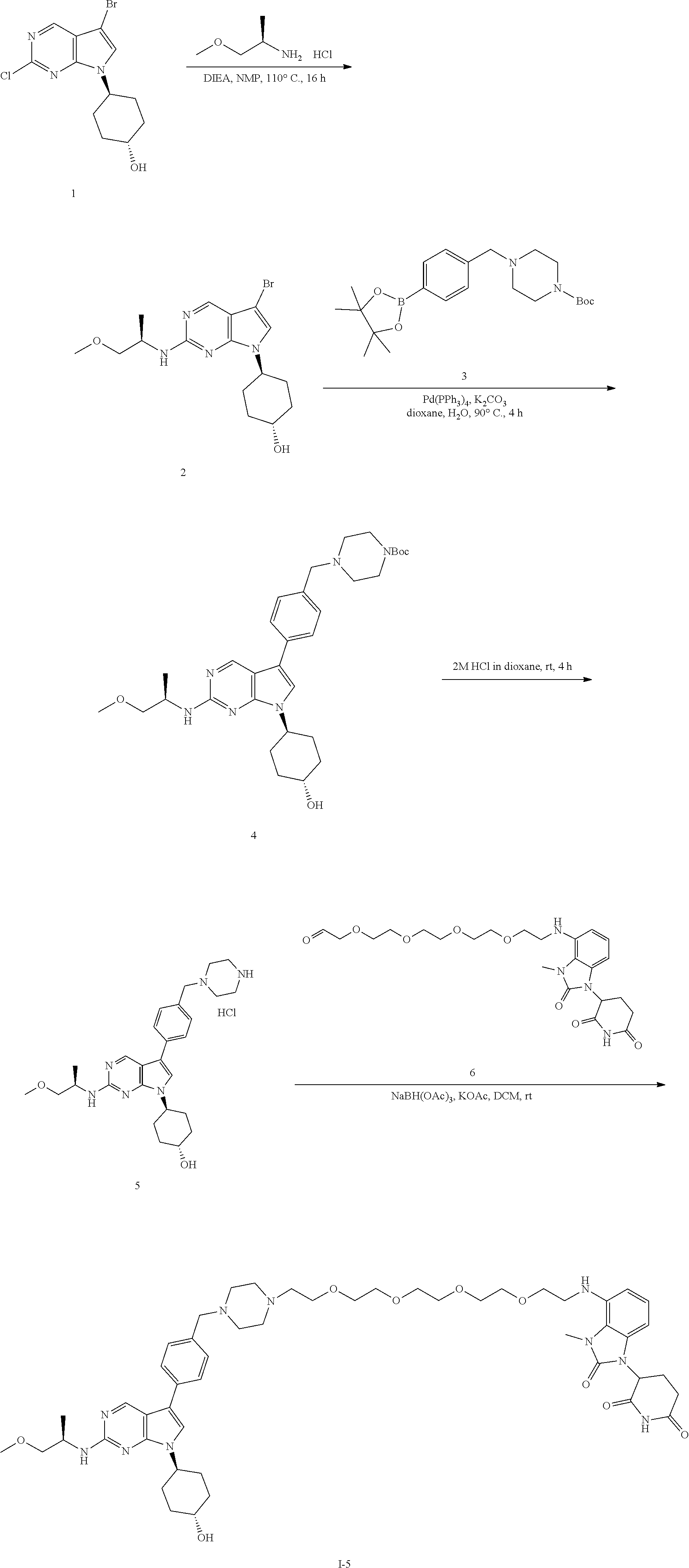

### Step 1: Trans-4-(5-bromo-2-[[(2R)-1-methoxypropan-2-yl]amino]-7H-pyrrolo[2,3-d]pyrimidin-7-yl)cyclohexan-1-ol

To a stirred solution of trans-4-[5-bromo-2-chloro-7H-pyrrolo[2,3-d]pyrimidin-7-yl]cyclohexan-1-ol (1.5 g, 4.537 mmol) and (2R)-1-methoxypropan-2-amine (2.02 g, 22.685 mmol) in NMP (16 mL) was added DIEA (5.86 g, 45.371 mmol) at room temperature. The reaction was stirred at 110° C. for 16 h. The resulting solution was cooled to room temperature and purified by reverse phase flash chromatography with the following conditions: Column: Spherical C¹⁸, 20-40 um, 330 g; Mobile Phase A: Water (plus 10 mM NH₄HCO₃); Mobile Phase B: ACN; Flow rate: 80 mL/min; Gradient: 43%-65% B gradient in 20 min; Detector: 254 nm. The fractions containing the desired product were collected at 50% B and concentrated under reduced pressure to afford trans-4-(5-bromo-2-[[(2R)-1-methoxypropan-2-yl] amino]-7H-pyrrolo[2,3-d]pyrimidin-7-yl)cyclohexan-1-ol (1.2 g, 69%) as a yellow solid. ¹H NMR (400 MHz, DMSO-d₆) δ 8.37 (s, 1H), 7.40 (s, 1H), 6.81 (d, J=8.0 Hz, 1H), 4.70 (s, 1H), 4.47-4.27 (m, 1H), 4.18 (t, J=7.1 Hz, 1H), 3.55-3.39 (m, 1H), 3.28 (s, 3H), 3.24 (d, J=8.0 Hz, 1H), 2.05-1.76 (m, 4H), 1.346-,1.22 (m, 4H), 1.15 (d, J=6.7 Hz, 3H). LC/MS (ESI, m/z): [(M+1)]⁺=383.2, 385.2

### Step 2: Tert-butyl 4-[[4-(2-[[(2R)-1-methoxypropan-2-yl]amino]-7-trans-4-hydroxycyclohexyl]-7H-pyrrolo[2,3-d]pyrimidin-5-yl)phenyl]methyl]piperazine-1-carboxylate

A mixture of trans-4-(5-bromo-2-[[(2R)-1-methoxypropan-2-yl]amino]-7H-pyrrolo[2,3-d]pyrimidin-7-yl)cyclohexan-1-ol (500 mg, 1.304 mmol), tert-butyl 4-(4-(4,4,5,5-tetramethyl-1,3,2-dioxaborolan-2-yl)benzyl)piperazine-1-carboxylate (626.55 mg, 1.957 mmol), Pd(PPh₃)₄ (150.74 mg, 0.130 mmol) and K₂CO₃ (540.87 mg, 3.913 mmol) in H₂O (3 mL) and dioxane (9.00 mL) was stirred for 4 h at 90° C. under nitrogen atmosphere. The mixture was allowed to cool down to room temperature and was filtered. The filtrate was purified by reverse phase flash chromatography with the following conditions: Column: WelFlash™ C18-I, 20-40 um, 330 g; Eluent A: Water (plus 10 mmol/L NH₄HCO₃); Eluent B: ACN; Gradient: 60%-70% B in 20 min; Flow rate: 80 mL/min; Detector: 220/254 nm; desired fractions were collected at 65% B and concentrated under reduced pressure to afford tert-butyl 4-[[4-(2-[[(2R)-1-methoxypropan-2-yl]amino]-7-[trans-4-hydroxycyclohexyl]-7H-pyrrolo[2,3-d]pyrimidin-5-yl)phenyl]methyl]piperazine-1-carboxylate (300 mg, 40%) as a yellow solid. ¹NMR (400 MHz, DMSO-d₆) δ 8.84 (s, 1H), 7.74-7.56 (m, 3H), 7.32 (d, J=7.9 Hz, 2H), 6.63 (d, J=8.0 Hz, 1H), 4.71 (d, J=4.4 Hz, 1H), 4.56-4.34 (m, 1H), 4.21 (p, J=6.7 Hz, 1H), 3.60-3.51 (m, 1H), 3.50-3.43 (m, 3H), 3.32 (s, 4H), 3.30 (s, 3H), 3.26 (dd, J=9.4, 6.9 Hz, 1H), 2.33 (t, J=5.0 Hz, 4H), 2.05-1.83 (m, 6H), 1.39 (s, 9H), 1.39-1.28 (m, 2H), 1.17 (d, J=6.6 Hz, 3H). LC/MS (ESI, m/z): [(M+1)]⁺=579.35

### Step 3: Trans-4-(2-[[(2R)-1-methoxypropan-2-yl]amino]-5-[4-[(piperazin-1-yl)methyl]phenyl]-7H-pyrrolo[2,3-d]pyrimidin-7-yl)cyclohexan-1-ol hydrochloride

To a stirred solution of tert-butyl 4-[[4-(2-[[(2R)-1-methoxypropan-2-yl]amino]-7-[trans-4-hydroxycyclohexyl]-7H-pyrrolo[2,3-d]pyrimidin-5-yl)phenyl]methyl]piperazine-1-carboxylate (600 mg, 1.04 mmol) in dioxane (10 mL) was added a solution of HCl (gas) in 1,4-dioxane (4 M, 10 mL) dropwise at room temperature under nitrogen atmosphere. The resulting mixture was stirred for 4 h at room temperature under nitrogen atmosphere. The resulting mixture was concentrated under vacuum. This resulted in trans-4-(2-[[(2R)-1-methoxypropan-2-yl]amino]-5-[4-[(piperazin-1-yl)methyl]phenyl]-7H-pyrrolo[2,3-d]pyrimidin-7-yl)cyclohexan-1-ol hydrochloride (500 mg, 94%) as a yellow solid. ¹H NMR (400 MHz, DMSO-d₆) δ 9.75 (br, 2H), 9.13 (s, 1H), 8.18 (s, 1H), 8.04 (s, 1H), 7.82 (d, J=7.9 Hz, 2H), 7.74 (d, J=7.8 Hz, 2H), 4.62-4.37 (m, 3H), 4.31 (s, 1H), 3.85-3.37 (m, 14H), 2.07-1.82 (m, 7H), 1.38 (d, J=14.9 Hz, 2H), 1.25 (d, J=6.6 Hz, 3H). LC/MS (ESI, m/z): [(M+1)]⁺=479.25

### Step 4: Trans-3-[4-[15-(4-[[4-(2-[[(2R)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid)

To a stirred solution of trans-4-(2-[[(2R)-1-methoxypropan-2-yl]amino]-5-[4-[(piperazin-1-yl)methyl]phenyl]-7H-pyrrolo[2,3-d]pyrimidin-7-yl)cyclohexan-1-ol hydrochloride (136 mg, 0.264 mmol), 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (130.04 mg, 0.264 mmol) and KOAc (103.65 mg, 1.056 mmol) in DCM (20 mL) was added NaBH(OAc)₃ (167.87 mg, 0.792 mmol) at room temperature. The resulting mixture was stirred for overnight at room temperature. The reaction was monitored by LCMS. Upon completion, the resulting mixture was concentrated under reduced pressure and was purified by Prep-HPLC with the following conditions (Column: XBridge Shield RP18 OBD Column, 5 um, 19×150 mm; Mobile Phase A: Water (0.1% FA), Mobile Phase B: ACN; Flow rate: 20 mL/min; Gradient: 10% B to 30% B in 7 min; 254 nm; Rt: 7 min) to afford trans-15-(4-[[4-(2-[[(2R)-1-methoxypropan-2-yl]amino]-7-[4-hydroxycyclohexyl]-7H-pyrrolo [2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid) (7.3 mg, 3%) as a light yellow solid. ¹H NMR (400 MHz, DMSO-d₆) δ 11.09 (s, 1H), 8.84 (s, 1H), 8.18 (s, 2H), 7.66-7.58 (m, 3H), 7.30 (d, J=7.8 Hz, 2H), 6.87 (t, J=8.0 Hz, 1H), 6.63 (d, J=8.1 Hz, 1H), 6.52 (d, J=7.9 Hz, 1H), 6.44 (d, J=8.2 Hz, 1H), 5.29 (m, 1H), 5.11-4.85 (m, 1H), 4.42 (t, J=4.7 Hz, 1H), 4.23-4.19 (m, 1H), 3.68-3.58 (m, 4H), 3.57-3.52 (m, 4H), 3.52-3.43 (m, 12H), 3.29 (s, 3H), 3.27-3.19 (m, 3H), 2.92-2.83 (m, 1H), 2.74-2.61 (m, 2H), 2.49-2.40 (m, 6H), 2.02-1.87 (m, 7H), 1.42-1.28 (m, 2H), 1.17 (d, J=6.6 Hz, 3H). LC/MS (ESI, m/z): [(M+1)]⁺=955.60

### EXAMPLE 28: Trans-3-[4-[15-(4-[[4-(2-[[(2S)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid) (I-4)

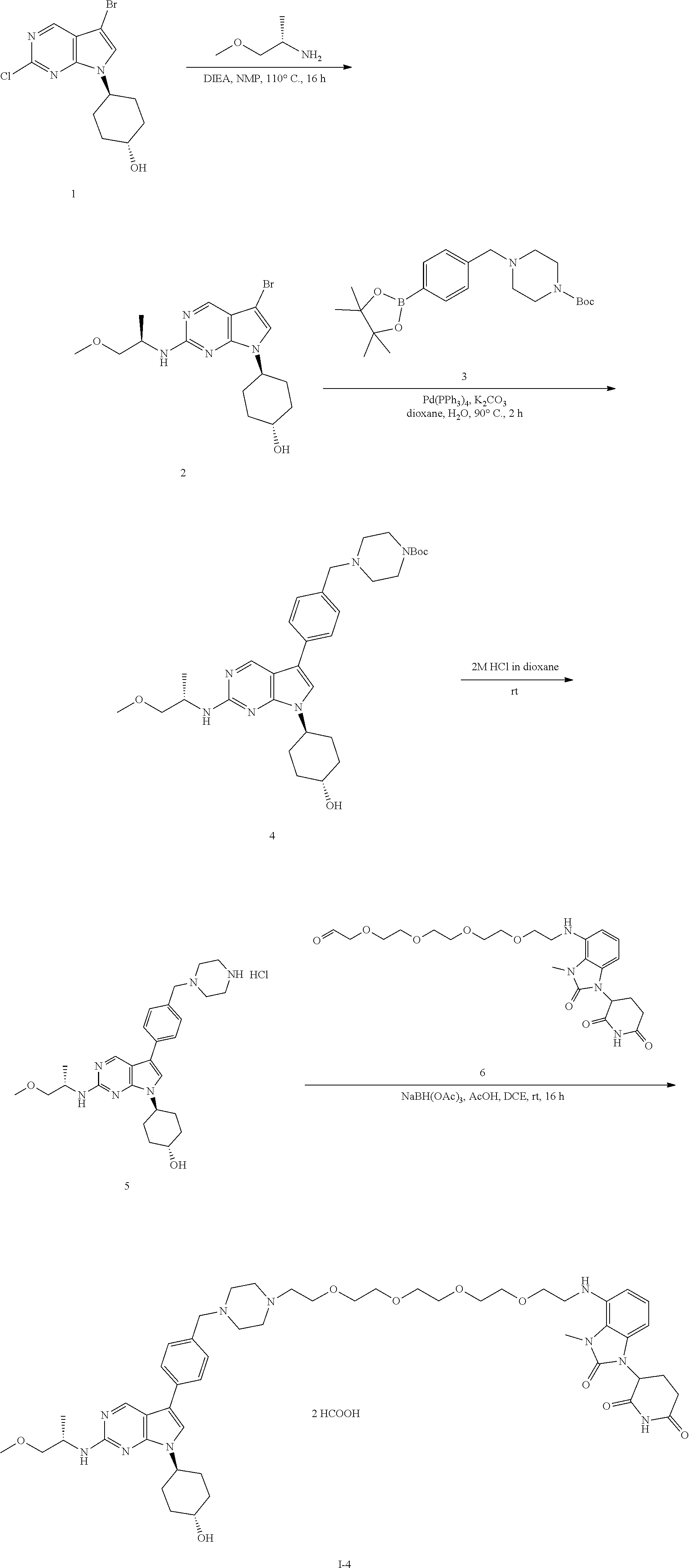

### Step 1: Trans-4-(5-bromo-2-[[(2S)-1-methoxypropan-2-yl]amino]-7H-pyrrolo[2,3-d]pyrimidin-7-yl)cyclohexan-1-ol

To a stirred solution of trans-4-[5-bromo-2-chloro-7H-pyrrolo[2,3-d]pyrimidin-7-yl]cyclohexan-1-ol (2 g, 6.049 mmol, 1 equiv) and (2S)-1-methoxypropan-2-amine (2.70 g, 30.247 mmol) in NMP (8 mL) was added DIEA (1.17 g, 9.074 mmol) at room temperature. The reaction was stirred at 110° C. for 16 h. The resulting mixture was concentrated under reduced pressure and was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 45% B and concentrated under reduced pressure to afford trans-4-(5-bromo-2-[[(2S)-1-methoxypropan-2-yl]amino]-7H-pyrrolo[2,3-d]pyrimidin-7-yl)cyclohexan-1-ol (2 g, 86%) as an orange solid. ¹H NMR (400 MHz, DMSO-d₆) δ 8.37 (s, 1H), 7.39 (s, 1H), 6.80 (d, J=8.0 Hz, 1H), 4.69 (d, J=4.4 Hz, 1H), 4.37 (dt, J=11.4, 6.5 Hz, 1H), 4.22-4.14 (m, 1H), 3.56-3.39 (m, 1H), 3.28 (s, 3H), 3.24 (dd, J=9.4, 6.8 Hz, 1H), 3.17 (d, J=5.2 Hz, 1H), 1.98-1.78 (m, 6H), 1.39-1.23 (m, 2H), 1.15 (d, J=6.6 Hz, 3H). LC/MS (ESI, m/z): [(M+1)]⁺=383.15, 384.15

### Step 2: Tert-butyl 4-[(4-[2-[(1-methoxypropan-2-yl)amino]-7-[trans-4-hydroxycyclohexyl]-7H-pyrrolo[2,3-d]pyrimidin-5-y]phenyl)methyl]piperazine-1-carboxylate

To a stirred solution of trans-4-[5-bromo-2-[(1-methoxypropan-2-yl)amino]-7H-pyrrolo[2,3-d]pyrimidin-7-yl]cyclohexan-1-ol (2 g, 5.218 mmol) and tert-butyl 4-[[4-(4,4,5,5-tetramethyl-1,3,2-dioxorolan-2-yl)phenyl]methyl]piperazine-1-carboxylate (3.15 g, 7.827 mmol) in dioxane (40 mL) and H₂O (4 mL) were added K₂CO₃ (1.08 g, 7.827 mmol) and Pd(PPh₃)₄ (0.30 g, 0.261 mmol) at room temperature under nitrogen atmosphere. The mixture was purged with nitrogen for 3 times and was stirred at 90° C. for 2 h. The mixture was allowed to cool down to room temperature, diluted with water (100 mL) and was extracted with EtOAc (3×100 mL). The combined organic layers was washed with brine (100 mL) and dried over anhydrous Na₂SO₄. After filtration, the filtrate was concentrated under reduced pressure. The residue was purified by reverse phase flash chromatography with the following conditions: Column: WelFlash™ C18-I, 20-40 μm, 330 g; Eluent A: Water (plus 10 mmol/L NH₄HCO₃); Eluent B: ACN; Gradient: 40%-60% B in 25 min; Flow rate: 80 mL/min; Detector: 220/254 nm; desired fractions were collected at 45% B and concentrated under reduced pressure to afford tert-butyl 4-[(4-[2-[(1-methoxypropan-2-yl)amino]-7-[trans-4-hydroxycyclohexyl]-7H-pyrrolo[2,3-d]pyrimidin-5-yl]phenyl)methyl]piperazine-1-carboxylate (1.8 g) as an yellow solid. ¹H NMR (400 MHz, DMSO-d₆) δ 8.84 (s, 1H), 7.67-7.58 (m, 3H), 7.32 (d, J=8.0 Hz, 2H), 6.62 (d, J=8.1 Hz, 1H), 4.71 (d, J=4.5 Hz, 1H), 4.47-4.36 (m, 1H), 4.22 (p, J=6.7 Hz, 1H), 3.60-3.50 (m, 1H), 3.52-3.42 (m, 3H), 3.36-3.21 (m, 8H), 3.17 (d, J=5.2 Hz, 1H), 2.39-2.22 (m, 4H), 2.05-1.81 (m,6H), 1.39 (s, 9H), 1.38-1.30 (m, 1H), 1.17 (d, J=6.6 Hz, 3H). LC/MS (ESI, m/z): [(M+1)]⁺=579.35

### Step 3: Trans-4-(2-[[(2S)-1-methoxypropan-2-y]amino]-5-[4-[(piperazin-1-yl)methyl]phenyl]-7H-pyrrolo[2,3-d]pyrimidin-7-yl)cyclohexan-1-ol

To a stirred solution of tert-butyl 4-[[4-(2-[[(2S)-1-methoxypropan-2-yl]amino]-7-[trans-4-hydroxycyclohexyl]-7H-pyrrolo[2,3-d]pyrimidin-5-yl)phenyl]methyl]piperazine-1-carboxylate (1.8 g, 3.110 mmol) in dioxane (5 mL) was added a solution of HCl in 1,4-dioxane (4 M, 5 mL) at room temperature. The reaction was stirred at room temperature for 2 h. The resulting mixture was concentrated under reduced pressure and to afford trans-4-(2-[[(2S)-1-methoxypropan-2-yl]amino]-5-[4-[(piperazin-1-yl)methyl]phenyl]-7H-pyrrolo[2,3-d]pyrimidin-7-yl)cyclohexan-1-ol (1.3 g, 87%) as a yellow solid. ¹H NMR (400 MHz, DMSO-d₆) δ 9.86 (br, 2H), 9.14 (s, 1H), 8.19 (s, 1H), 8.12 (s, 1H), 7.83 (d, J=8.0 Hz, 2H), 7.75 (d, J=8.0 Hz, 2H), 4.54-4.26 (m, 4H), 3.62-3.38 (m, 12H), 3.17 (s, 3H), 2.03-1.93 (m, 6H), 1.49-1.31 (m, 2H), 1.25 (d, J=6.7 Hz, 3H). LC/MS (ESI, m/z): [(M+1)]⁺=479.40

### Step 4: Trans-3-[4-[15-(4-[[4-(2-[[(2S)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid)

To a stirred solution of trans-4-(2-[[(2S)-1-methoxypropan-2-yl]amino]-5-[4-[(piperazin-1-yl)methyl]phenyl]-7H-pyrrolo[2,3-d]pyrimidin-7-yl)cyclohexan-1-ol (126 mg, 0.263 mmol), 14-[[1-(2,6-dioxopiperidin-3-yl)-3-methyl-2-oxo-2,3-dihydro-1H-1,3-benzodiazol-4-yl]amino]-3,6,9,12-tetraoxatetradecanal (129.66 mg, 0.263 mmol) and KOAc (103.34 mg, 1.053 mmol) in DCM (20 mL) was added NaBH(OAc)₃ (167.38 mg, 0.790 mmol) at room temperature. The resulting mixture was stirred for overnight. The reaction was monitored by LCMS. The resulting mixture was concentrated under reduced pressure and was purified by Prep-HPLC with the following conditions (Column: XBridge Shield RP18 OBD Column, 5 um, 19×150 mm; Mobile Phase A: Water (0.1% FA), Mobile Phase B: ACN; Flow rate: 20 mL/min; Gradient: 10% B to 30% B in 7 min; 254 nm; Rt: 7 min) to afford trans-3-[4-[15-(4-[[4-(2-[[(2S)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-3-methyl-2-oxo-2,3-dihydro-1H-1,3-benzodiazol-1-yl]piperidine-2,6-dione bis(formic acid) (13 mg, 5%) as a light yellow solid. ¹H NMR (400 MHz, DMSO-d₆) δ 11.09 (s, 1H), 8.84 (s, 1H), 8.16 (s, 2H), 7.74-7.57 (m, 3H), 7.30 (d, J=8.1 Hz, 2H), 6.87 (t, J=8.0 Hz, 1H), 6.64 (d, J=8.1 Hz, 1H), 6.52 (d, J=7.9 Hz, 1H), 6.45 (d, J=8.2 Hz, 1H), 5.29 (dd, J=12.7, 5.3 Hz, 1H), 5.00 (s, 1H), 4.71 (s, 1H), 4.42 (t, J=11.5 Hz, 1H), 4.21 (p, J=6.7 Hz, 1H), 3.62 (d, J=9.1 Hz, 5H), 3.55 (m, 4H), 3.52-3.42 (m, 16H), 3.29 (s, 3H), 3.27-3.20 (m, 3H), 2.97-2.79 (m, 1H), 2.77-2.56 (m, 2H), 2.49-2.31 (m, 8H), 2.08-1.81 (m, 6H), 1.35 (d, J=12.0 Hz, 2H), 1.17 (d, J=6.6 Hz, 3H). LC/MS (ESI, m/z): [(M+1)]⁺=955.60

### EXAMPLE 29: Trans-2-(2,6-dioxopiperidin-3-yl)-4-[15-(4-[[4-(2-[[(2S)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-2,3-dihydro-1H-isoindole-1,3-dione (I-11)

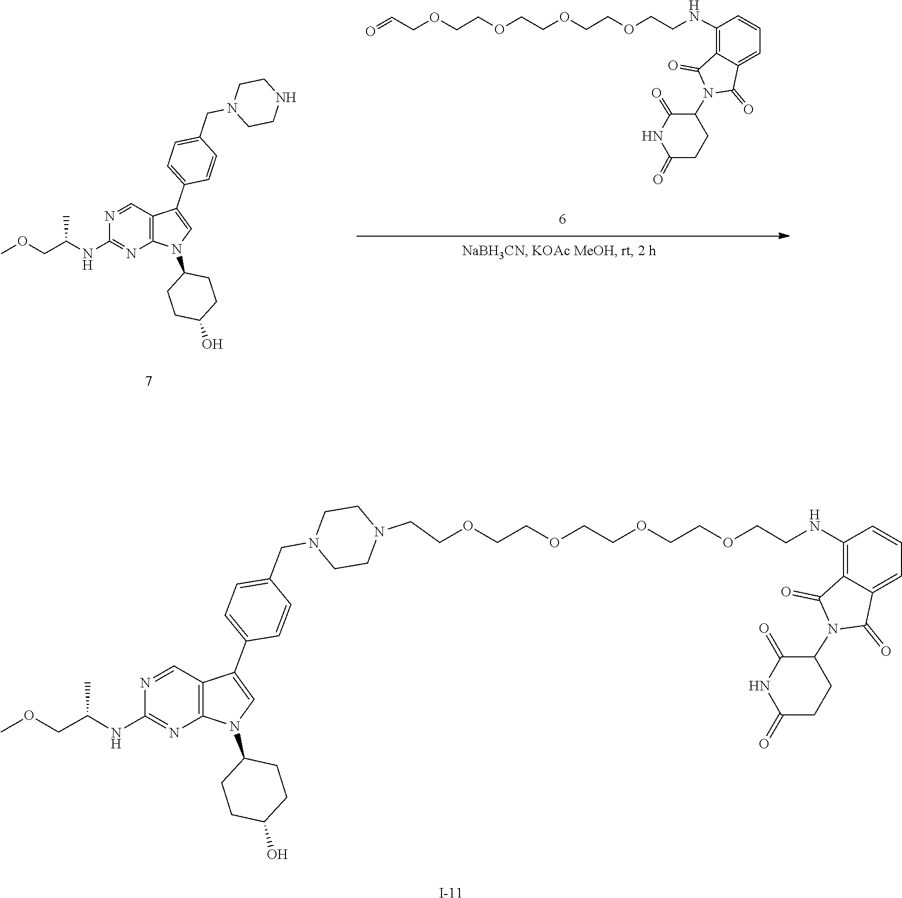

To a stirred solution of 14-[[2-(2,6-dioxopiperidin-3-yl)-1,3-dioxo-2,3-dihydro-1H-isoindol-4-yl]amino]-3,6,9,12-tetraoxatetradecanal (73.93 mg, 0.150 mmol) and 14-[[2-(2,6-dioxopiperidin-3-yl)-1,3-dioxo-2,3-dihydro-1H-isoindol-4-yl]amino]-3,6,9,12-tetraoxatetradecanal (73.93 mg, 0.150 mmol), KOAc (49.21 mg, 0.501 mmol) in MeOH (30 mL) was added NaBH₃CN (23.63 mg, 0.376 mmol) at room temperature. The reaction was allowed to react for 2 h. The reaction was monitored by LCMS. Upon completion, the solution was condensed under reduced pressure and was purified by Prep-HPLC with the following conditions (Column: XBridge Shield RP18 OBD Column, 5 um, 19×150 mm; Mobile Phase A: Water (10 mmol/L NH₄HCO₃+0.1% NH₃.H₂O), Mobile Phase B: ACN; Flow rate: 25 mL/min; Gradient: 32% B to 45% B in 8 min; 254/220 nm; Rt: 7.63 min) to afford trans-2-(2,6-dioxopiperidin-3-yl)-4-[15-(4-[[4-(2-[[(2S)-1-methoxypropan-2-yl]amino]-7-[4-hydroxycyclohexyl]-7H-pyrrolo[2,3-d]pyrimidin-5-yl)phenyl]methyl]piperazin-1-yl)-4,7,10,13-tetraoxa-1-azapentadecan-1-yl]-2,3-dihydro-1H-isoindole-1,3-dione (34 mg, 28%) as a yellow solid. ¹H NMR (400 MHz, DMSO-d₆) δ 11.11 (s, 1H), 8.84 (s, 1H), 7.70-7.52 (m, 4H), 7.30 (d, J=7.9 Hz, 2H), 7.13 (d, J=8.6 Hz, 1H), 7.04 (d, J=7.0 Hz, 1H), 6.61 (d, J=7.6 Hz, 2H), 5.06 (dd, J=13.0, 5.4 Hz, 1H), 4.70 (d, J=4.5 Hz, 1H), 4.42 (t, J=11.8 Hz, 1H), 4.21 (dt, J=13.8, 6.9 Hz, 1H), 3.61 (t, J=5.4 Hz, 2H), 3.59-3.52 (m, 2H), 3.47 (m, 15H), 3.30 (s, 3H), 3.26 (dd, J=9.4, 6.9 Hz, 2H), 2.96-2.82 (m, 1H), 2.64-2.53 (m, 5H), 2.41 (m, 8H), 2.08-1.83 (m, 8H), 1.44-1.30 (m, 2H), 1.17 (d, J=6.7 Hz, 3H). LC/MS (ESI, m/z): [(M+1)]⁺=954.45

### EXAMPLE 30: Trans-4-([4-[2-(butylamino)-7-[4-hydroxycyclohexyl]-7H-pyrrolo[2,3-d]pyrimidin-5-yl]phenyl]methyl)-N-[3-(3-[3-[1-(2,6-dioxopiperidin-3-yl)-3-methyl-2-oxo-2,3-dihydro-1H-1,3-benzodiazol-5-yl]propoxy]propoxy)propyl]piperazine-1-carboxamide (I-10)

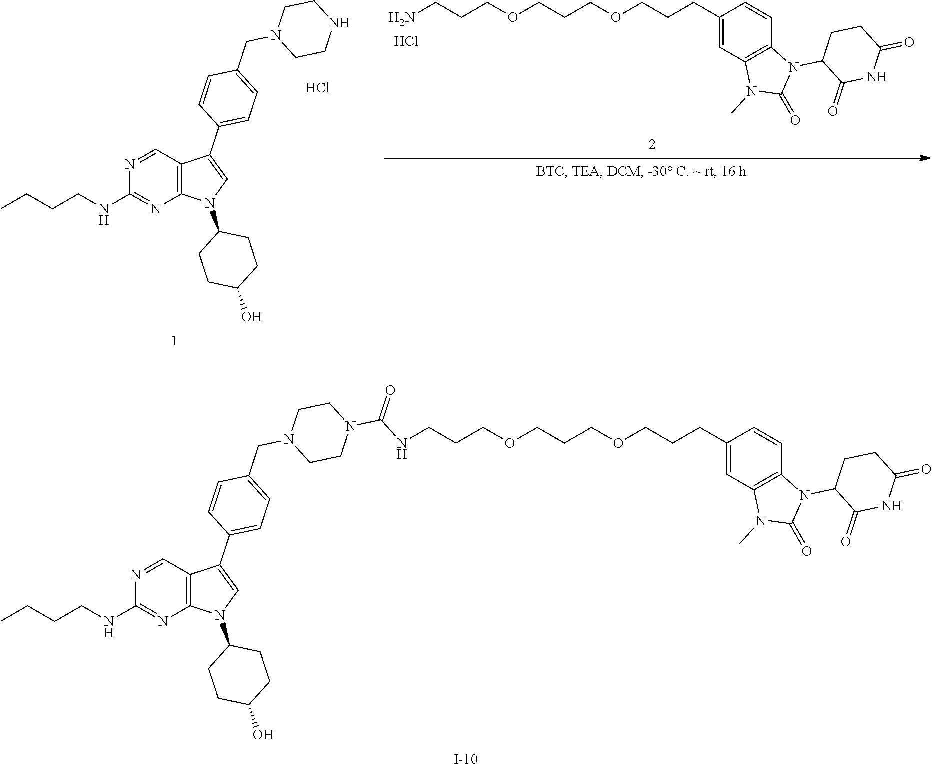

To a stirred solution of trans-4-[2-(butylamino)-5-[4-[(piperazin-1-yl)methyl]phenyl]-7H-pyrrolo[2,3-d]pyrimidin-7-yl]cyclohexan-1-ol hydrochloride (100 mg, 0.200 mmol), TEA (202.74 mg, 2.004 mmol) and 3-(5-[3-[3-(3-aminopropoxy)propoxy]propyl]-3-methyl-2-oxo-2,3-dihydro-1H-1,3-benzodiazol-1-yl)piperidine-2,6-dione (86.66 mg, 0.200 mmol) in DCM (10 mL) was added a solution of bis(trichloromethyl) carbonate (17.85 mg, 0.060 mmol) in DCM (5 mL) dropwise at −30° C. under nitrogen atmosphere. The solution was allowed to react for 16 h at room temperature. The reaction was monitored by LCMS. Upon completion, the solution was concentrated under reduced pressure and was purified by reverse flash chromatography with the following conditions: column, C18 silica gel; mobile phase, ACN in 10 mmol FA aq, 25% to 45% gradient in 20 min; detector, UV 254 nm to give trans-4-([4-[2-(butylamino)-7-[4-hydroxycyclohexyl]-7H-pyrrolo[2,3-d]pyrimidin-5-yl]phenyl]methyl)-N-[3-(3-[3-[1-(2,6-dioxopiperidin-3-yl)-3-methyl-2-oxo-2,3-dihydro-1H-1,3-benzodiazol-5-yl]propoxy]propoxy)propyl]piperazine-1-carboxamide (30 mg, 16%) as a white solid. ¹H NMR (400 MHz, DMSO-d₆) δ 11.09 (s, 1H), 8.83 (s, 1H), 7.70-7.53 (m, 3H), 7.32 (d, J=7.9 Hz, 2H), 7.10-6.94 (m, 2H), 6.94-6.77 (m, 2H), 6.42 (t, J=5.5 Hz, 1H), 5.34 (dd, J=12.7, 5.4 Hz, 1H), 4.72-4.68 (m, 1H), 4.51-4.37 (m, 1H), 3.53 (dd, J=9.8, 5.0 Hz, 1H), 3.47 (s, 2H), 3.44-3.39 (m, 4H), 3.39-3.30 (m, 12H), 3.30-3.24 (m, 4H), 3.06 (q, J=6.5 Hz, 2H), 2.96-2.83 (m, 1H), 2.78-2.57 (m, 4H), 2.36-2.28 (m, 4H), 2.04-1.94 (m, 4H), 1.94-1.86 (m, 1H), 1.86-1.77 (m, 1H), 1.73 (p, J=6.4 Hz, 2H), 1.62 (q, J=6.2, 5.7 Hz, 2H), 1.55 (q, J=7.3 Hz, 2H), 1.44-1.30 (m, 4H), 0.93 (t, J=7.3 Hz, 3H). LC/MS (ESI, m/z): [(M+1)]⁺=921.55

### EXAMPLE 31: Trans-4-[4-([4-[2-(butylamino)-7-[4-hydroxycyclohexyl]-7H-pyrrolo[2,3-d]pyrimidin-5-yl]phenyl]methyl)piperazin-1-yl]-N-[3-(3-[3-[1-(2,6-dioxopiperidin-3-yl)-3-methyl-2-oxo-2,3-dihydro-1H-1,3-benzodiazol-5-yl]propoxy]propoxy)propyl]butanamide bis(formic acid) (I-9)

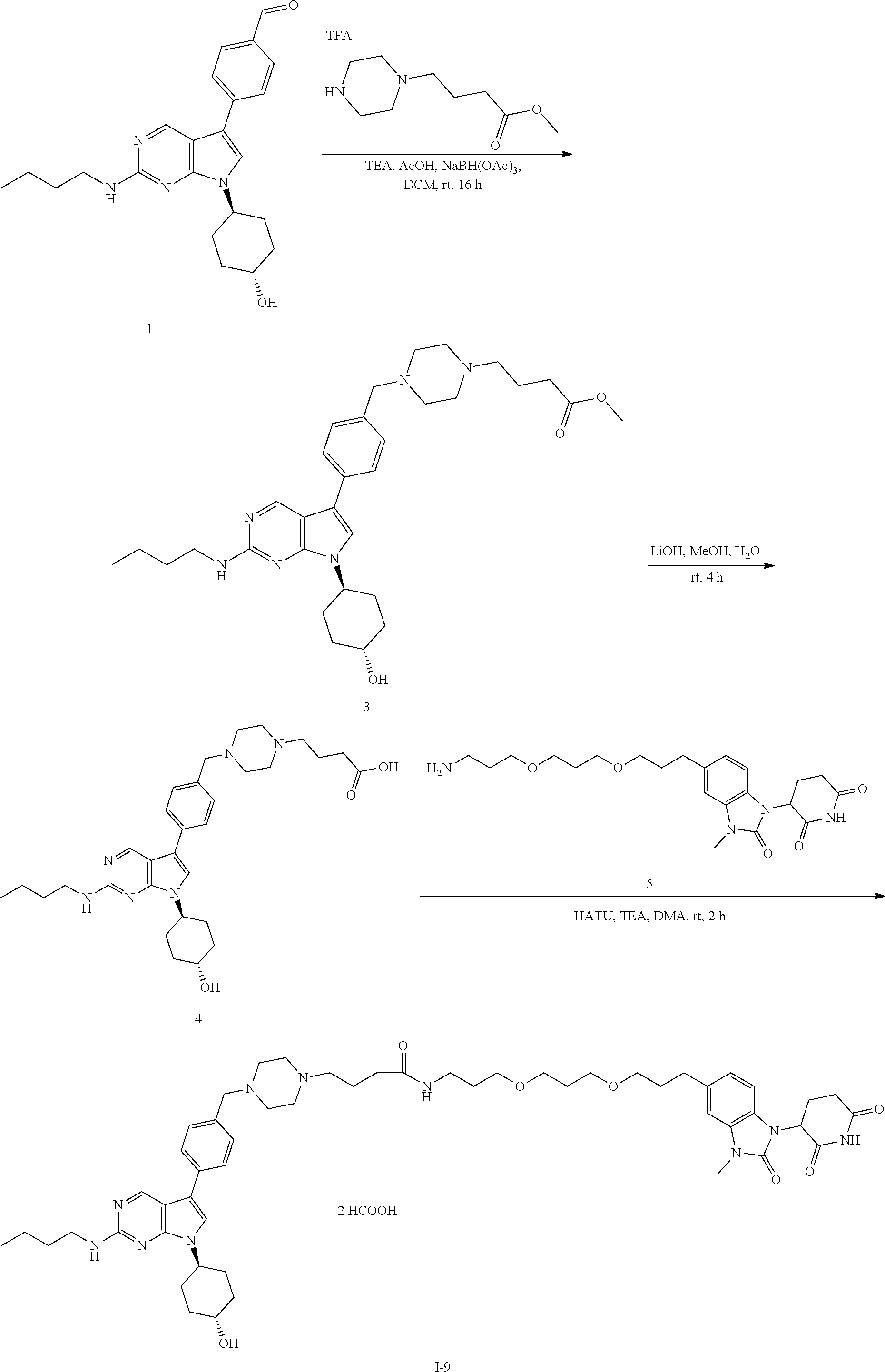

### Step 1: Trans-methyl-4-(4-(4-(2-(butylamino)-7-(4-hydroxycyclohexyl)-7H-pyrrolo[2,3-d]pyrimidin-5-yl)benzyl)piperazin-1-yl)butanoate

To a stirred solution of trans-4-[2-(butylamino)-7-[4-hydroxycyclohexyl]-7H-pyrrolo[2,3-d]pyrimidin-5-yl]benzaldehyde (750 mg, 1.911 mmol), TEA (1.93 g, 19.108 mmol) and methyl 4-[4-(2,2,2-trifluoroacetyl)piperazin-1-yl]butanoate (2.16 g, 7.643 mmol) in DCM (50 mL) was added AcOH (1.72 g, 28.662 mmol) at room temperature under nitrogen atmosphere. The solution was stirred for 30 min at room temperature. Then NaBH(OAc)₃ (2.03 g, 9.554 mmol) was added to the reaction. The reaction was allowed to react for 2 h at room temperature. The reaction was monitored by LCMS. Upon completion, the reaction was condensed under reduced pressure and was purified by reverse flash chromatography with the following conditions: column, C¹⁸ silica gel; mobile phase, ACN in 10 mmol NH₄HCO₃ aqueous solution, 35% to 55% gradient in 20 min; detector, UV 254 nm to give methyl trans-4-[4-([4-[2-(butylamino)-7-[4-hydroxycyclohexyl]-7H-pyrrolo[2,3-d]pyrimidin-5-yl]phenyl]methyl)piperazin-1-yl]butanoate (750 mg, 70%) as a white solid. ¹H NMR (400 MHz, DMSO-d₆) δ 8.82 (s, 1H), 7.61 (d, J=7.9 Hz, 2H), 7.56 (s, 1H), 7.30 (d, J=7.8 Hz, 2H), 6.82 (t, J=5.8 Hz, 1H), 4.69 (d, J=4.4 Hz, 1H), 4.47-4.37 (m, 1H), 3.58 (s, 3H), 3.54 (t, J=7.2 Hz, 1H), 3.44 (s, 2H), 3.34 (d, J=7.5 Hz, 2H), 2.49-2.20 (m, 12H), 205-1.84 (m, 6H), 1.66 (p, J=7.1 Hz, 2H), 1.56 (p, J=7.2 Hz, 2H), 1.37 (h, J=8.4, 7.8 Hz, 4H), 0.93 (t, J=7.3 Hz, 3H). LC/MS (ESI, m/z): [(M+1)]⁺=563.40

### Step 2: Trans-4-(4-(4-(2-(butylamino)-7-(4-hydroxycyclohexyl)-7H-pyrrolo[2,3-d]pyrimidin-5-yl)benzyl)piperazin-1-yl)butanoic acid

To a stirred solution of methyl trans-4-[4-([4-[2-(butylamino)-7-[4-hydroxycyclohexyl]-7H-pyrrolo[2,3-d]pyrimidin-5-yl]phenyl]methyl)piperazin-1-yl]butanoate (750 mg, 1.333 mmol) in MeOH (50 mL) was added NaOH (2 M, 3.33 mL) dropwise at 0° C. The solution was stirred for 4 h at room temperature. The reaction was monitored by LCMS. Upon completion, the solution was neutralized to pH 5 with 3 M HCl (aq.) and was condensed under reduced pressure. The residue was purified by reverse flash chromatography with the following conditions: column, C18 silica gel; mobile phase, ACN in 10 mmol NH₄HCO₃ aqueous solution, 25% to 50% gradient in 25 min; detector, UV 254 nm to give trans-4-[4-([4-[2-(butylamino)-7-[4-hydroxycyclohexyl]-7H-pyrrolo[2,3-d]pyrimidin-5-yl]phenyl]methyl)piperazin-1-yl]butanoic acid (650 mg, 89%) as a white solid. ¹H NMR (400 MHz, DMSO-d₆) δ 8.82 (s, 1H), 7.64-7.60 (m, 2H), 7.58 (s, 1H), 7.31 (d, J=8.0 Hz, 2H), 6.84 (t, J=5.8 Hz, 1H), 4.70 (s, 1H), 4.51-4.37 (m, 1H), 3.60-3.49 (s, 1H), 3.46 (s, 2H), 3.41-3.25 (m, 1H), 2.47-2.34 (m, 8H), 2.31 (t, J=7.0 Hz, 2H), 2.23 (t, J=7.1 Hz, 2H), 2.03-1.93 (m, 4H), 1.93-1.83 (m, 3H), 1.64 (p, J=7.1 Hz, 2H), 1.55 (q, J=7.2 Hz, 2H), 1.45-1.27 (m, 4H), 0.93 (t, J=7.3 Hz, 3H). LC/MS (ESI, m/z): [(M+1)]⁺=549.25

### Step 3: Trans-4-[4-([4-[2-(butylamino)-7-[4-hydroxycyclohexyl]-7H-pyrrolo[2,3-d]pyrimidin-5-yl]phenyl]methyl)piperazin-1-yl]-N-[3-(3-[3-[1-(2,6-dioxopiperidin-3-yl)-3-methyl-2-oxo-2,3-dihydro-1H-1,3-benzodiazol-5-yl]propoxy]propoxy)propyl]butanamide bis(formic acid)

To a stirred solution of trans-4-[4-([4-[2-(butylamino)-7-[4-hydroxycyclohexyl]-7H-pyrrolo[2,3-d]pyrimidin-5-yl]phenyl]methyl)piperazin-1-yl]butanoic acid (50 mg, 0.091 mmol), 3-(5-[3-[3-(3-aminopropoxy)propoxy]propyl]-3-methyl-2-oxo-2,3-dihydro-1H-1,3-benzodiazol-1-yl)piperidine-2,6-dione (39.41 mg, 0.091 mmol) and TEA (18.44 mg, 0.182 mmol) in DMA (5 mL) was added HATU (41.58 mg, 0.109 mmol) at 0° C. under nitrogen atmosphere. The solution was stirred for 2 h at room temperature. The reaction was monitored by LCMS. Upon completion, the residue was purified by Prep-HPLC with the following conditions (Column: XBridge Shield RP18 OBD Column, 5 um, 19×150 mm; Mobile Phase A: Water (0.1% FA), Mobile Phase B: ACN; Flow rate: 25 mL/min; Gradient: 10% B to 30% B in 7 min; 254 nm; Rt: 7 min) to give trans-4-[4-([4-[2-(butylamino)-7-[4-hydroxycyclohexyl]-7H-pyrrolo[2,3-d]pyrimidin-5-yl]phenyl]methyl)piperazin-1-yl]-N-[3-(3-[3-[1-(2,6-dioxopiperidin-3-yl)-3-methyl-2-oxo-2,3-dihydro-1H-1,3-benzodiazol-5-yl]propoxy]propoxy)propyl]butanamide bis(formic acid) (50 mg, 52%) as an off white solid. ¹H NMR (400 MHz, DMSO-d₆) δ 11.09 (s, 1H), 8.82 (s, 1H), 8.19 (s, 2H), 7.74 (t, J=5.7 Hz, 1H), 7.62 (d, J=7.8 Hz, 2H), 7.57 (s, 1H), 7.30 (d, J=7.8 Hz, 2H), 7.05-6.97 (m, 2H), 6.89-6.80 (m, 2H), 5.34 (m, 1H), 4.42 (m, 1H), 3.60-3.49 (m, 1H), 3.46 (s, 2H), 3.44-3.39 (m, 4H), 3.40-3.34 (m, 5H), 3.32 (s, 4H), 3.11-3.03 (m, 2H), 2.97-2.84 (m, 1H), 2.77-2.55 (m, 5H), 2.47-2.29 (m, 8H), 2.29-2.21 (m, 2H), 2.09-2.02 (m, 2H), 2.02-1.93 (m, 4H), 1.93-1.85 (m, 2H), 1.84-1.77 (m, 2H), 1.76-1.70 (m, 2H), 1.68-1.51 (m, 6H), 1.43-1.29 (m, 4H), 0.93 (t, J=7.3 Hz, 3H). LC/MS (ESI, m/z): [(M+1)]⁺=964.60

### EXAMPLE 32: Trans-4-[4-([4-[2-(butylamino)-7-[4-hydroxycyclohexyl]-7H-pyrrolo[2,3-d]pyrimidin-5-yl]phenyl]methyl)piperazin-1-yl]-N-[3-(3-[3-[1-(2,6-dioxopiperidin-3-yl)-3-methyl-2-oxo-2,3-dihydro-1H-1,3-benzodiazol-4-yl]propoxy]propoxy)propyl]butanamide formic acid (I-8)

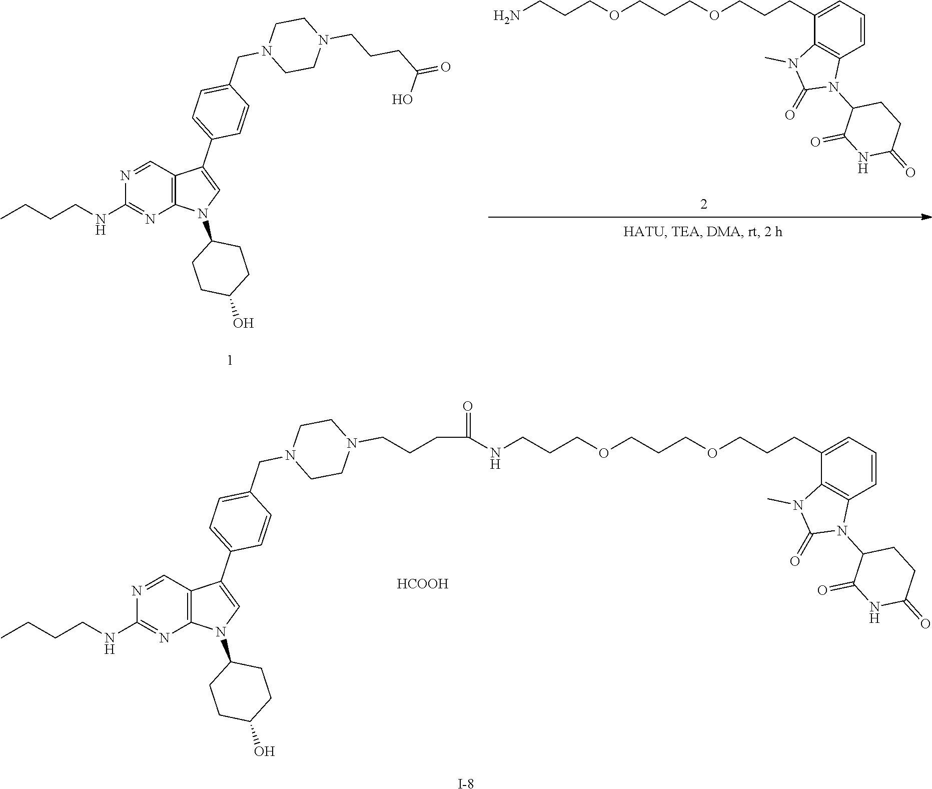

To a stirred solution of trans-4-[4-([4-[2-(butylamino)-7-[4-hydroxycyclohexyl]-7H-pyrrolo[2,3-d]pyrimidin-5-yl]phenyl]methyl)piperazin-1-yl]butanoic acid (50 mg, 0.091 mmol), TEA (18.44 mg, 0.182 mmol) and 3-(4-[3-[3-(3-aminopropoxy)propoxy]propyl]-3-methyl-2-oxo-2,3-dihydro-1H-1,3-benzodiazol-1-yl)piperidine-2,6-dione (39.41 mg, 0.091 mmol) in DMA (5 mL) was added HATU (41.58 mg, 0.109 mmol) at 0° C. under nitrogen atmosphere. The solution was stirred for 2 h at room temperature. The reaction was monitored by LCMS. Upon completion, the residue was purified by Pre-HPLC with the following conditions (Column: XBridge Shield RP18 OBD Column, 5 um, 19×150 mm; Mobile Phase A: Water (0.1% FA), Mobile Phase B: ACN; Flow rate: 20 mL/min; Gradient: 10% B to 30% B in 7 min; 254 nm; Rt: 7 min) to give trans-4-[4-([4-[2-(butyl amino)-7-[4-hydroxycyclohexyl]-7H-pyrrolo[2,3-d]pyrimidin-5-yl]phenyl]methyl)piperazin-1-yl]-N-[3-(3-[3-[1-(2,6-dioxopiperidin-3-yl)-3-methyl-2-oxo-2,3-dihydro-1H-1,3-benzodiazol-4-yl]propoxy]propoxy)propyl]butanamide formic acid (50 mg, 54%) as a white solid. ¹H NMR (400 MHz, DMSO-d₆) δ 11.10 (s, 1H), 8.82 (s, 1H), 8.16 (s, 1H), 7.75 (t, J=5.6 Hz, 1H), 7.62 (d, J=7.9 Hz, 2H), 7.57 (s, 1H), 7.30 (d, J=7.9 Hz, 2H), 7.04-6.91 (m, 2H), 6.91-6.80 (m, 2H), 5.37 (dd, J=12.6, 5.4 Hz, 1H), 4.41 (dq, J=11.3, 5.1, 4.1 Hz, 1H), 3.60-3.50 (m, 4H), 3.50-3.38 (m, 10H), 3.38-3.28 (m, 6H), 3.07 (q, J=6.5 Hz, 2H), 2.95 (dd, J=9.3, 6.5 Hz, 2H), 2.91-2.83 (m, 1H), 2.78-2.56 (m, 2H), 2.48-2.34 (m, 8H), 2.29 (t, J=7.3 Hz, 2H), 2.06 (t, J=7.3 Hz, 2H), 2.03-1.94 (m, 4H), 1.90 (d, J=4.8 Hz, 1H), 1.82 (dd, J=8.9, 6.4 Hz, 1H), 1.74 (p, J=6.4 Hz, 2H), 1.68-1.51 (m, 6H), 1.43-1.29 (m, 4H), 0.93 (t, J=7.3 Hz, 3H). LC/MS (ESI, m/z): [(M+1)]⁺=963.60

### EXAMPLE 33: Trans-3-[5-[3-(3-[3-[([4-[2-(butylamino)-7-[4-hydroxycyclohexyl]-7H-pyrrolo[2,3-d]pyrimidin-5-yl]phenyl]methyl)amino]propoxy]propoxy)propyl]-3-methyl-2-oxo-2,3-dihydro-1H-1,3-benzodiazol-1-yl]piperidine-2,6-dione formic acid (I-7)

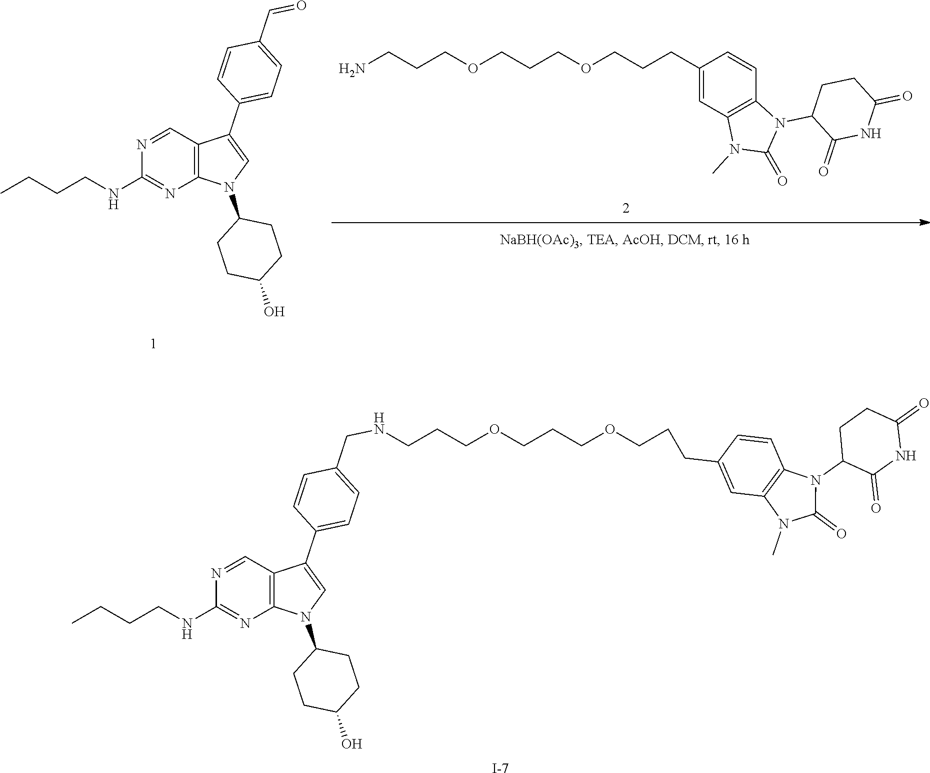

To a stirred solution of trans-4-[2-(butylamino)-7-[4-hydroxycyclohexyl]-7H-pyrrolo[2,3-d]pyrimidin-5-yl]benzaldehyde (50 mg, 0.127 mmol), TEA (38.67 mg, 0.382 mmol) and 3-(5-[3-[3-(3-aminopropoxy)propoxy]propyl]-3-methyl-2-oxo-2,3-dihydro-1H-1,3-benzodiazol-1-yl)piperidine-2,6-dione (66.12 mg, 0.153 mmol) in DCM (5 mL) were added AcOH (30.60 mg, 0.510 mmol) at 0° C. under nitrogen atmosphere. The solution was stirred for 30 min at room temperature. Then NaBH(OAc)₃ (134.99 mg, 0.637 mmol) was added to the reaction. The mixture was allowed to react for additional 16 h at room temperature. The reaction was monitored by LCMS. Upon completion, the resulting mixture was concentrated under reduced pressure and was purified by Prep-HPLC with the following conditions (Column: XBridge Shield RP18 OBD Column 30×150 mm, 5 um; Mobile Phase A: Water (10 mmol/L FA), Mobile Phase B: ACN; Flow rate: 60 mL/min; Gradient: 35% B to 45% B in 9 min; 254 nm; Rt: 8.5 min) to afford trans-3-[5-[3-(3-[3-[([4-[2-(butylamino)-7-[4-hydroxycyclohexyl]-7H-pyrrolo[2,3-d]pyrimidin-5-yl]phenyl]methyl)amino]propoxy]propoxy)propyl]-3-methyl-2-oxo-2,3-dihydro-1H-1,3-benzodiazol-1-yl]piperidine-2,6-dione (20 mg, 19%) as a white solid. ¹H NMR (400 MHz, DMSO-d₆) δ 8.82 (s, 1H), 8.27 (s, 1H), 7.62 (d, J=7.9 Hz, 2H), 7.57 (s, 1H), 7.36 (d, J=7.9 Hz, 2H), 7.07-6.96 (m, 2H), 6.90-6.79 (m, 2H), 5.33 (dd, J=12.7, 5.3 Hz, 1H), 4.52-4.35 (m, 1H), 3.75 (s, 2H), 3.60-3.46 (m, 1H), 3.45-3.34 (m, 9H), 3.33-3.28 (m, 6H), 2.99-2.83 (m, 1H), 2.75-2.66 (m, 1H), 2.66-2.57 (m, 4H), 2.03-1.92 (m, 4H), 1.92-1.83 (m, 3H), 1.82 -1.65 (m, 5H), 1.56 (p, J=7.2 Hz, 2H), 1.36 (h, J=8.2, 7.7 Hz, 4H), 0.93 (t, J=7.3 Hz, 3H). LC/MS (ESI, m/z): [(M/2 +1)]⁺=405.55

### EXAMPLE 34: Trans-3-[4-[3-(3-[3-[([4-[2-(butylamino)-7-[4-hydroxycyclohexyl]-7H-pyrrolo[2,3-d]pyrimidin-5-yl]phenyl]methyl)amino]propoxy]propoxy)propyl]-3-methyl-2-oxo-2,3-dihydro-1H-1,3-benzodiazol-1-yl]piperidine-2,6-dione (I-6)

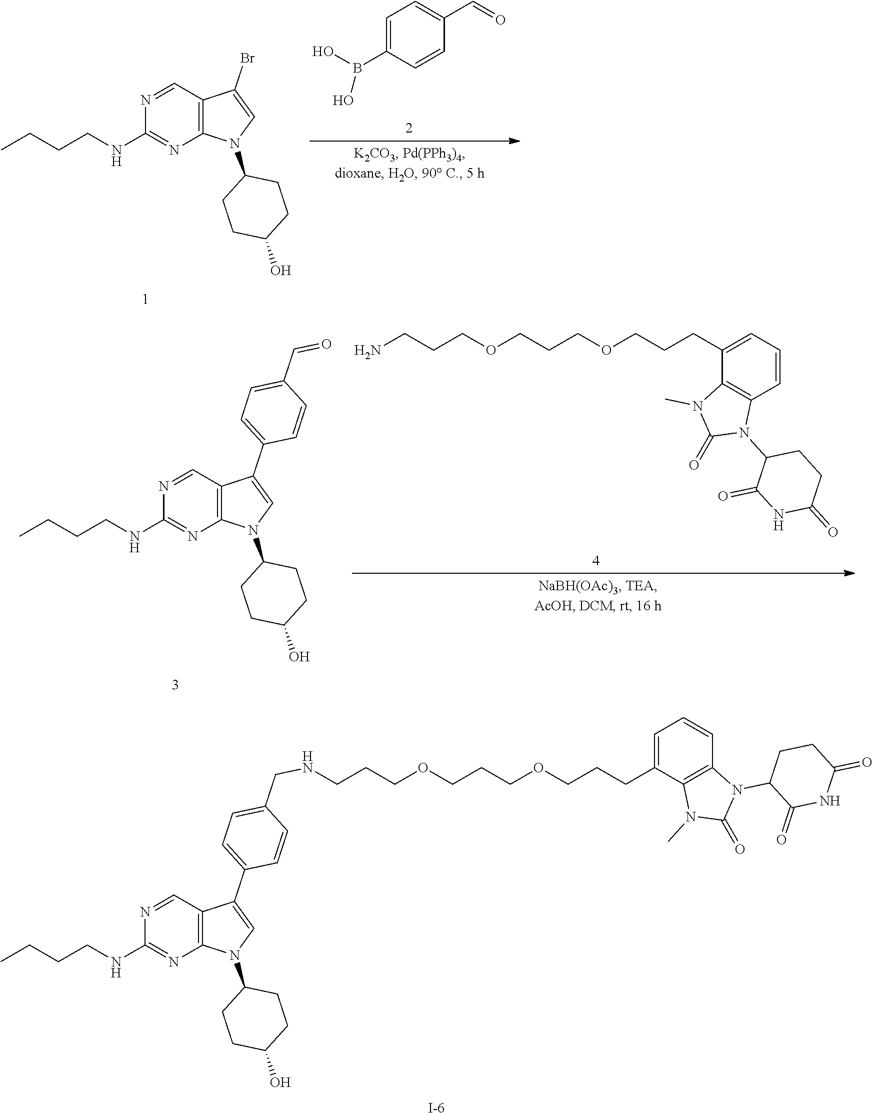

### Step 1: Trans-4-(2-(butylamino)-7-(4-hydroxycyclohexyl)-7H-pyrrolo[2,3-d]pyrimidin-5-yl)benzaldehyde

To a stirred mixture of trans-4-[5-bromo-2-(butylamino)-7H-pyrrolo[2,3-d]pyrimidin-7-yl]cyclohexan-1-ol (1.2 g, 3.267 mmol), (4-formylphenyl)boronic acid (0.59 g, 3.921 mmol) and K₂CO₃ (1.35 g, 9.801 mmol) in dioxane (9 mL)/H₂O (3 mL) was added Pd(PPh₃)₄ (0.38 g, 0.327 mmol) at room temperature under nitrogen atmosphere. The mixture was purged with nitrogen 3 times and allowed to react for 3 h at 90° C. The reaction was monitored by LCMS. Upon completion, the mixture was allowed to cool down to room temperature and diluted with water (50 mL), extracted with EtOAc (3×50 mL). The combined organic layers were dried over anhydrous Na₂SO₄. After filtration, the filtrate was concentrated under reduced pressure. The residue was purified by reverse flash chromatography with the following conditions: column, C18 silica gel; mobile phase, ACN in 10 mM NH₄CO₃ aqueous solution, 40% to 60% gradient in 20 min; detector, UV 254 nm to give trans-4-[2-(butylamino)-7-[4-hydroxycyclohexyl]-7H-pyrrolo[2,3-d]pyrimidin-5-yl]benzaldehyde (0.91 g, 71%) as an off white solid. ¹H NMR (400 MHz, DMSO-d₆) δ 9.98 (s, 1H), 8.96 (s, 1H), 8.09-7.99 (m, 1H), 7.98-7.86 (m, 4H), 6.96 (t, J=5.8 Hz, 1H), 4.71 (d, J=4.5 Hz, 1H), 4.49-4.40 (m, 1H), 3.64-3.50 (m, 1H), 3.35-3.30 (m, 2H), 2.06-1.85 (m, 6H), 1.57 (p, J=7.2 Hz, 2H), 1.37 (h, J=7.4 Hz, 4H), 0.93 (t, J=7.3 Hz, 3H) LC/MS (ESI, m/z): [(M+1)]⁺=393.25

### Step 2: Trans-3-[4-[3-(3-[3-[([4-[2-(butylamino)-7-[4-hydroxycyclohexyl]-7H-pyrrolo[2,3-d]pyrimidin-5-yl]phenyl]methyl)amino]propoxy]propoxy)propyl]-3-methyl-2-oxo-2,3-dihydro-1H-1,3-benzodiazol-1-yl]piperidine-2,6-dione

To a stirred solution of trans-4-[2-(butylamino)-7-[4-hydroxycyclohexyl]-7H-pyrrolo[2,3-d]pyrimidin-5-yl]benzaldehyde (50 mg, 0.127 mmol), TEA (38.67 mg, 0.382 mmol) and 3-(4-[3-[3-(3-aminopropoxy)propoxy]propyl]-3-methyl-2-oxo-2,3-dihydro-1H-1,3-benzodiazol-1-yl)piperidine-2,6-dione (66.12 mg, 0.153 mmol) in DCM (5 mL) were added AcOH (30.60 mg, 0.510 mmol) at 0° C. under nitrogen atmosphere. The solution was stirred for 30 min at room temperature. Then NaBH(OAc)₃ (134.99 mg, 0.637 mmol) was added to the reaction and the mixture was allowed to react for additional 16 h at room temperature. The reaction was monitored by LCMS. Upon completion, the resulting mixture was concentrated under reduced pressure and was purified by Prep-HPLC with the following conditions (Column: XBridge Shield RP18 OBD Column 30×150 mm, 5 um; Mobile Phase A: Water (10 mmol/L FA), Mobile Phase B: ACN; Flow rate: 60 mL/min; Gradient: 35% B to 45% B in 9 min; 254 nm; Rt: 8.5 min) to afford trans-3-[4-[3-(3-[3-[([4-[2-(butylamino)-7-[4-hydroxycyclohexyl]-7H-pyrrolo[2,3-d]pyrimidin-5-yl]phenyl]methyl)amino]propoxy]propoxy)propyl]-3-methyl-2-oxo-2,3-dihydro-1H-1,3-benzodiazol-1-yl]piperidine-2,6-dione (20 mg, 19%) as a white solid. ¹H NMR (400 MHz, DMSO-d₆) δ 11.10 (s, 1H), 8.82 (s, 1H), 7.63 (d, J=7.9 Hz, 2H), 7.57 (s, 1H), 7.36 (d, J=7.9 Hz, 2H), 7.08-6.91 (m, 2H), 6.84 (dd, J=6.3, 2.5 Hz, 2H), 5.36 (dd, J=12.6, 5.4 Hz, 1H), 4.69 (s, 1H), 4.53-4.32 (m, 1H), 3.76 (s, 2H), 3.55 (s, 4H), 3.48-3.33 (m, 12H), 2.98-2.82 (m, 3H), 2.77-2.57 (m, 4H), 2.04-1.93 (m, 4H), 1.89 (s, 2H), 1.74 (m, 5H), 1.56 (p, J=7.1 Hz, 2H), 1.36 (h, J=7.6 Hz, 4H), 0.93 (t, J=7.3 Hz, 3H). LC/MS (ESI, m/z): [(M+1)]⁺=809.50

### EXAMPLE 35: 2-Amino-5-[1-(4-[[3-(3-[3-[1-(2,6-dioxopiperidin-3-yl)-3-methyl-2-oxo-2,3-dihydro-1H-1,3-benzodiazol-5-yl]propoxy]propoxy)propyl]carbamoyl]butyl)-1H-pyrazol-4-yl]-N-[(1S,2S)-2-([4-[(4-methylpiperazin-1-yl)methyl]-[1,1-biphenyl]-4-yl]methoxy)cyclopentyl]pyridine-3-carboxamide (I-2)

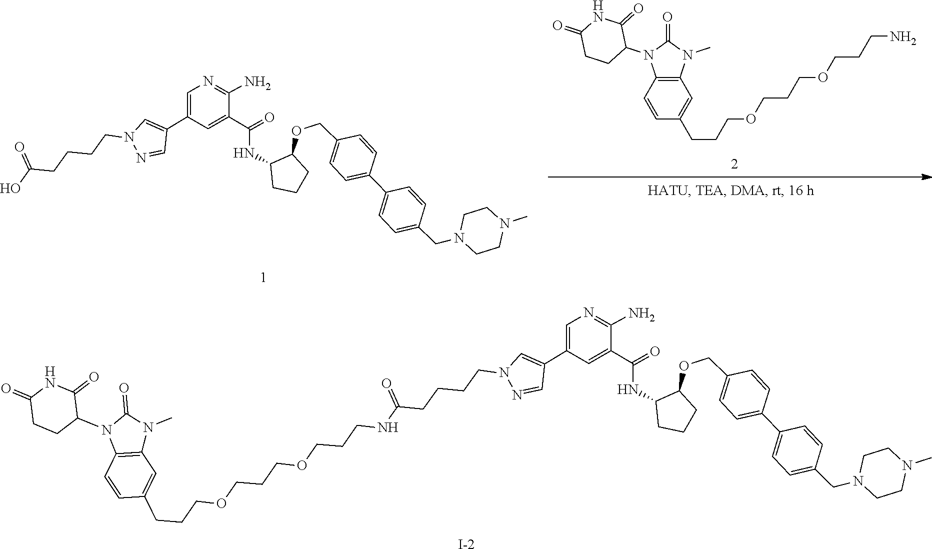

To a stirred solution of 3-(5-[3-[3-(3-aminopropoxy)propoxy]propyl]-3-methyl-2-oxo-2,3-dihydro-1H-1,3-benzodiazol-1-yl)piperidine-2,6-dione hydrochloride (28.17 mg, 0.060 mmol) and TEA (18.24 mg, 0.180 mmol) in DMA (2 mL) were added 5-[4-(6-amino-5-[[(1S,2S)-2-([4-[(4-methylpiperazin-1-yl)methyl]-[1,1-biphenyl]-4-yl]methoxy)cyclopentyl]carbamoyl]pyridin-3-yl)-1H-pyrazol-1-yl]pentanoic acid (40 mg, 0.060 mmol) at room temperature under nitrogen atmosphere. To the above solution was added HATU (29.69 mg, 0.078 mmol) at 0° C. under nitrogen atmosphere. The resulting mixture was stirred for overnight at room temperature under nitrogen atmosphere. The reaction was monitored by LCMS. The reaction mixture was purified by reverse flash chromatography with the following conditions: Column: WelFlash™ C18-I, 20-40 μm, 120 g; Eluent A: Water (plus 10 mmol/L formic acid); Eluent B: ACN; Gradient: 25%-45% B in 20 min; Flow rate: 50 mL/min; Detector: 220/254 nm; desired fractions were collected at 36% B and concentrated under reduced pressure to afford 2-amino-5-[1-(4-[[3-(3-[3-[1-(2,6-dioxopiperidin-3-yl)-3-methyl-2-oxo-2,3-dihydro-1H-1,3-benzodiazol-5-yl]propoxy]propoxy)propyl]carbamoyl]butyl)-1H-pyrazol-4-yl]-N-[(1S,2S)-2-([4-[(4-methylpiperazin-1-yl)methyl]-[1,1-biphenyl]-4-yl]methoxy)cyclopentyl]pyridine-3-carboxamide (14.4 mg, 22%) as a white solid. ¹H NMR (400 MHz, Methanol-d4) δ 8.26 (d, J=2.2 Hz, 1H), 8.01 (d, J=2.3 Hz, 1H), 7.90 (s, 1H), 7.79 (s, 1H), 7.59-7.49 (m, 4H), 7.45-7.36 (m, 4H), 7.08-6.98 (m, 2H), 6.94 (dd, J=8.1, 1.5 Hz, 1H), 5.31 (dd, J=12.5, 5.4 Hz, 1H), 4.75-4.62 (m, 2H), 4.50-4.39 (m, 1H), 4.13 (t, J=6.9 Hz, 2H), 4.05-3.94 (m, 1H), 3.72 (s, 2H), 3.52-3.38 (m, 9H), 3.33-3.30 (m, 4H), 3.24 (t, J=6.9 Hz, 4H), 3.04-2.66 (m, 12H), 2.25-2.11 (m, 4H), 2.10-1.99 (m, 1H), 1.92-1.70 (m, 11H), 1.68-1.50 (m, 3H). LC/MS (ESI, m/z): [(M+1)]⁺=1080.60

### EXAMPLE 36: 2-Amino-N-[(1S,2S)-2-[(4-[[4-(3-[[3-(3-[3-[1-(2,6-dioxopiperidin-3-yl)-3-methyl-2-oxo-2,3-dihydro-1H-1,3-benzodiazol-5-yl]propoxy]propoxy)propyl]carbamoyl]propyl)piperazin-1-yl]methyl]-[1,1-biphenyl]-4-yl)methoxy]cyclopentyl]-5-(1-methyl-1H-pyrazol-4-yl)pyridine-3-carboxamide formic acid (I-1)

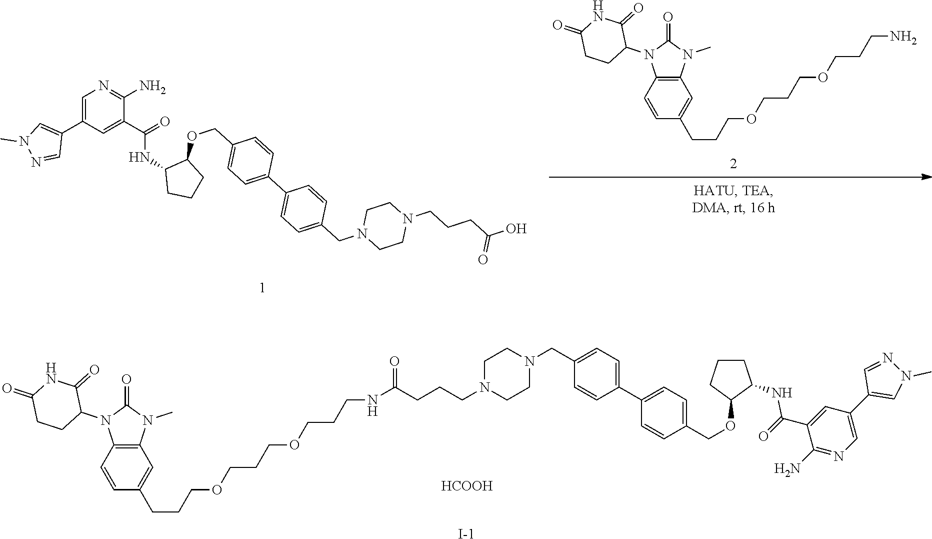 [SMILES: Cn1cc(-c2cnc(N)c(C(=O)N[C@H]3CCC[C@@H]3OCc3ccc(-c4ccc(CN5CCN(CCCC(=O)O)CC5)cc4)cc3)c2)cn1 ; Cn1c(=O)n(C2CCC(=O)NC2=O)c2ccc(CCCOCCCOCCCN)cc21 ; Cn1cc(-c2cnc(N)c(C(=O)N[C@H]3CCC[C@@H]3OCc3ccc(-c4ccc(CN5CCN(CCCC(=O)NCCCOCCCOCCCc6ccc7c(c6)n(C)c(=O)n7C6CCC(=O)NC6=O)CC5)cc4)cc3)c2)cn1 ; "HCOOH" ; [I-]]

To a stirred solution of 3-(5-[3-[3-(3-aminopropoxy)propoxy]propyl]-3-methyl-2-oxo-2,3-dihydro-1H-1,3-benzodiazol-1-yl)piperidine-2,6-dione hydrochloride (28.78 mg, 0.061 mmol) and TEA (18.63 mg, 0.184 mmol) in DMA (2 mL) was added 4-(4-[[4-([[(1S,2S)-2-[2-amino-5-(1-methyl-1H-pyrazol-4-yl)pyridine-3-amido]cyclopentyl]oxy]methyl)-[1,1-biphenyl]-4-yl]methyl]piperazin-1-yl)butanoic acid (40 mg, 0.061 mmol) at room temperature. To the above solution was added HATU (30.33 mg, 0.080 mmol) at 0° C. The resulting mixture was stirred for overnight at room temperature under nitrogen atmosphere. The reaction was monitored by LCMS. The reaction mixture was purified by Prep-HPLC with the following conditions (Column: XBridge Shield RP18 OBD Column, 5 um, 19×150 mm; Mobile Phase A: Water (0.1% FA), Mobile Phase B: ACN; Flow rate: 25 mL/min; Gradient: 10% B to 40% B in 7 min; 254 nm; Rt: 6.67 min) to afford 2-amino-N-[(1S,2S)-2-[(4-[[4-(3-[[3-(3-[3-[1-((2,6-dioxopiperidin-3-yl)-3-methyl-2-oxo-2,3-dihydro-1H-1,3-benzodiazol-5-yl]propoxy]propoxy)propyl]carbamoyl]propyl)piperazin-1-yl]methyl]-[1,1-biphenyl]-4-yl)methoxy]cyclopentyl]-5-(1-methyl-1H-pyrazol-4-yl)pyridine-3-carboxamide formic acid (32.9 mg, 48%) as a white solid. ¹H NMR (400 MHz, Methanol-d4) δ 8.40 (s, 1H), 8.26 (d, J=2.3 Hz, 1H), 7.99 (d, J=2.2 Hz, 1H), 7.85 (s, 1H), 7.77 (d, J=0.8 Hz, 1H), 7.54 (d, J=7.9 Hz, 4H), 7.42 (dd, J=12.8, 8.1 Hz, 4H), 7.10-6.89 (m, 3H), 5.32 (dd, J=12.5, 5.4 Hz, 1H), 4.78-4.64 (m, 2H), 4.50-4.38 (m, 1H), 3.99 (q, J=5.1 Hz, 1H), 3.90-3.87 (m, 3H), 3.75 (s, 2H), 3.57-3.42 (m, 10H), 3.40 (d, J=2.0 Hz, 3H), 3.27 (t, J=7.0 Hz, 2H), 3.20-3.00 (m, 3H), 2.99-2.87 (m, 4H), 2.86-2.70 (m, 6H), 2.36 (t, J=6.8 Hz, 2H), 2.25-2.13 (m, 2H), 2.12-2.01 (m, 1H), 1.99-1.72 (m, 11H), 1.69-1.54 (m, 1H). LC/MS (ESI, m/z): [(M+1)]⁺=1066.65

### EXAMPLE 37: (2S,4R)-4-Hydroxy-1-[(2S)-2-([2-[(5-[4-[(4-[2-[(1-methoxypropan-2-yl)amino]-7-(trans-4-hydroxycyclohexyl)-7H-pyrrolo[2,3-d]pyrimidin-5-yl]phenyl)methyl]piperazin-1-yl]pentyl)amino]ethyl]amino)-3,3-dimethylbutanoyl]-N-[[4-(4-methyl-1,3-thiazol-5-yl)phenyl]methyl]pyrrolidine-2-carboxamide (I-3)

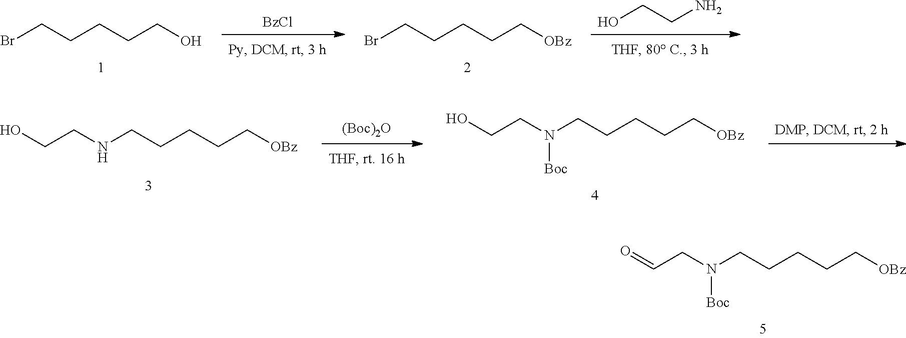

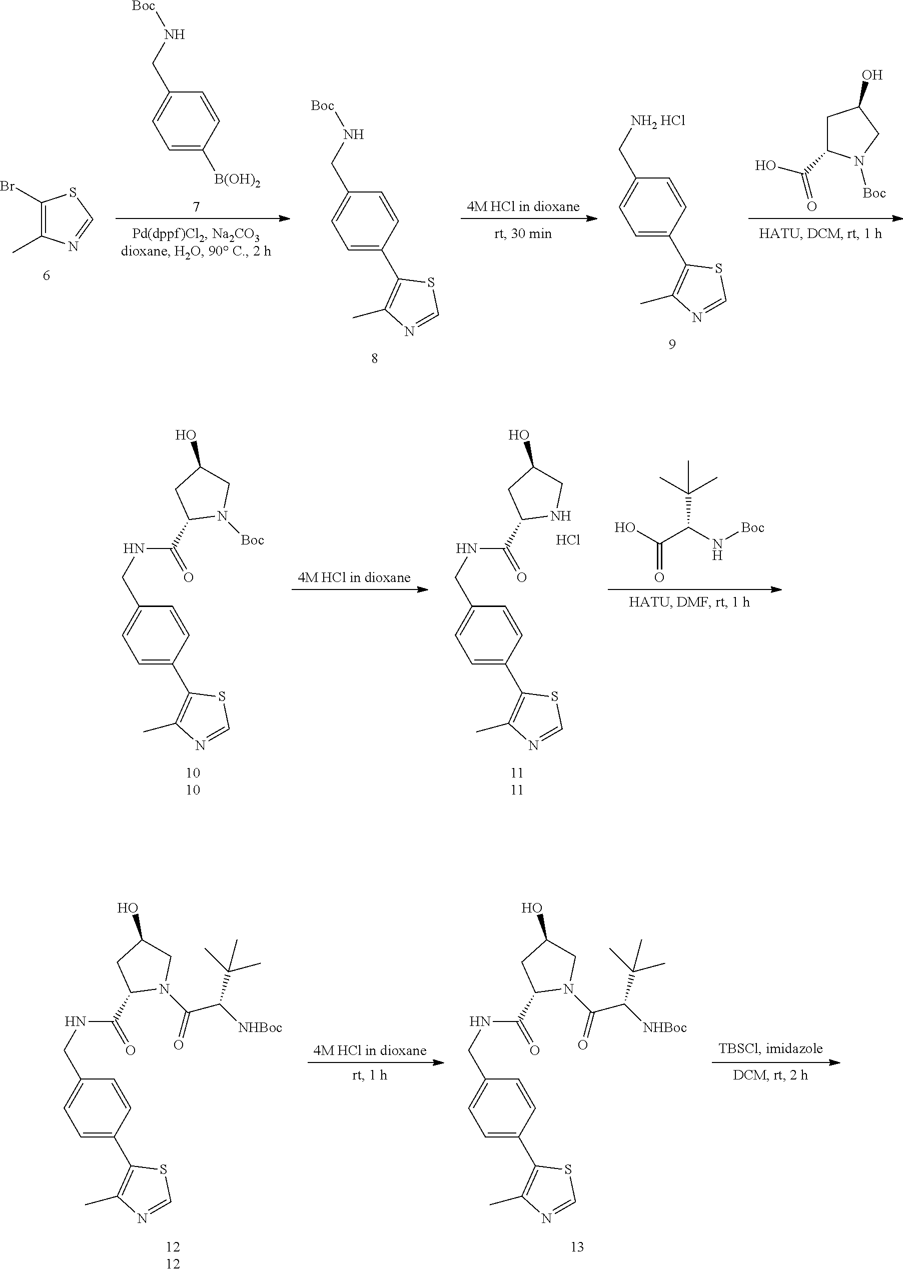

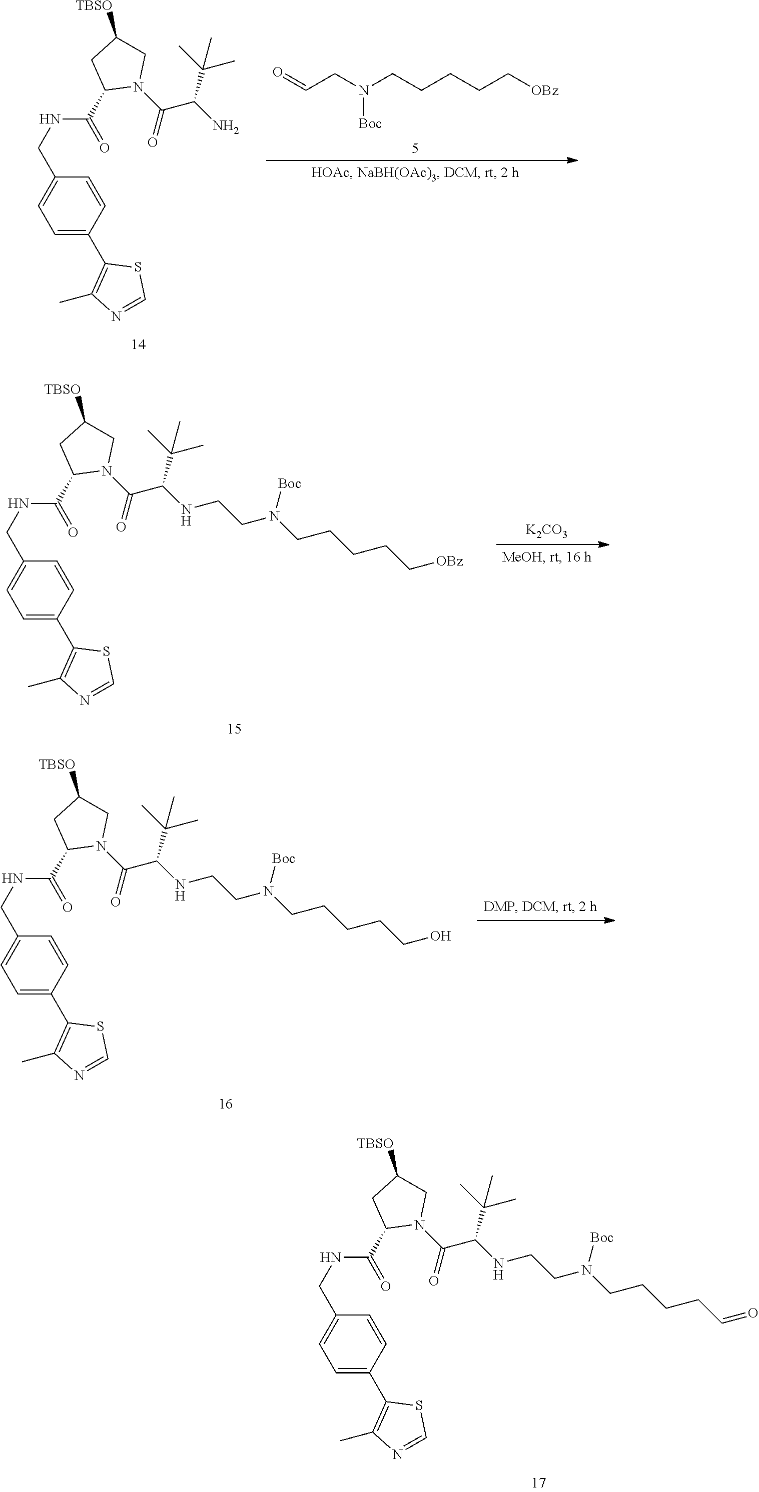

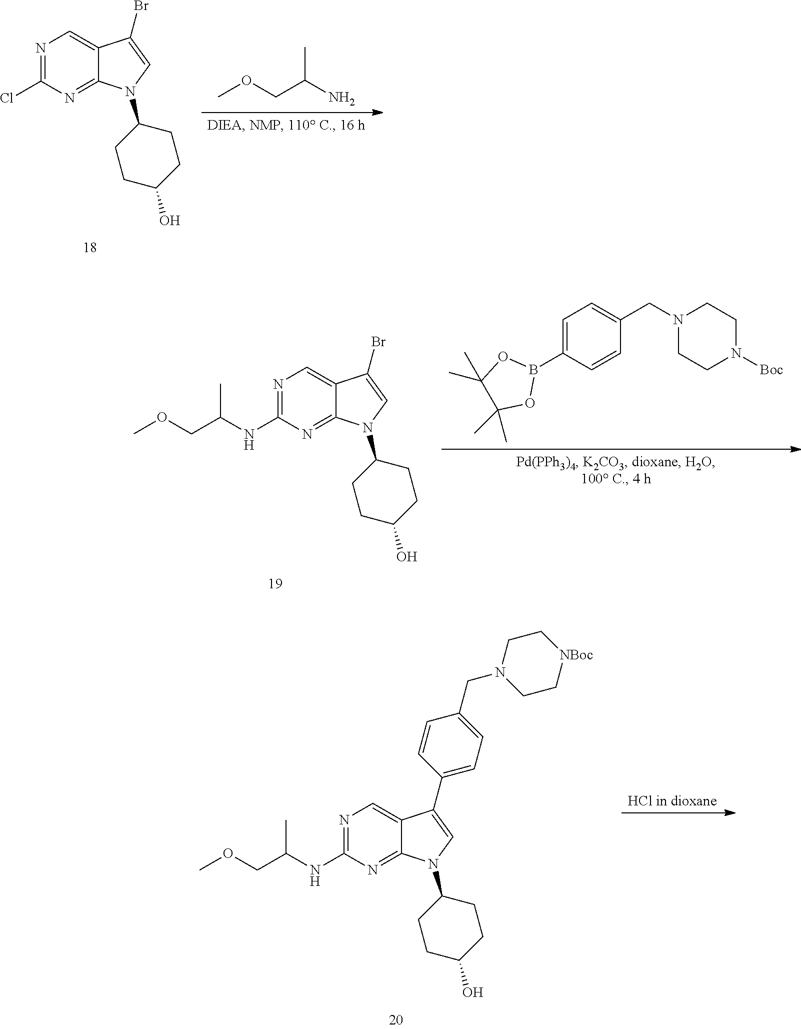

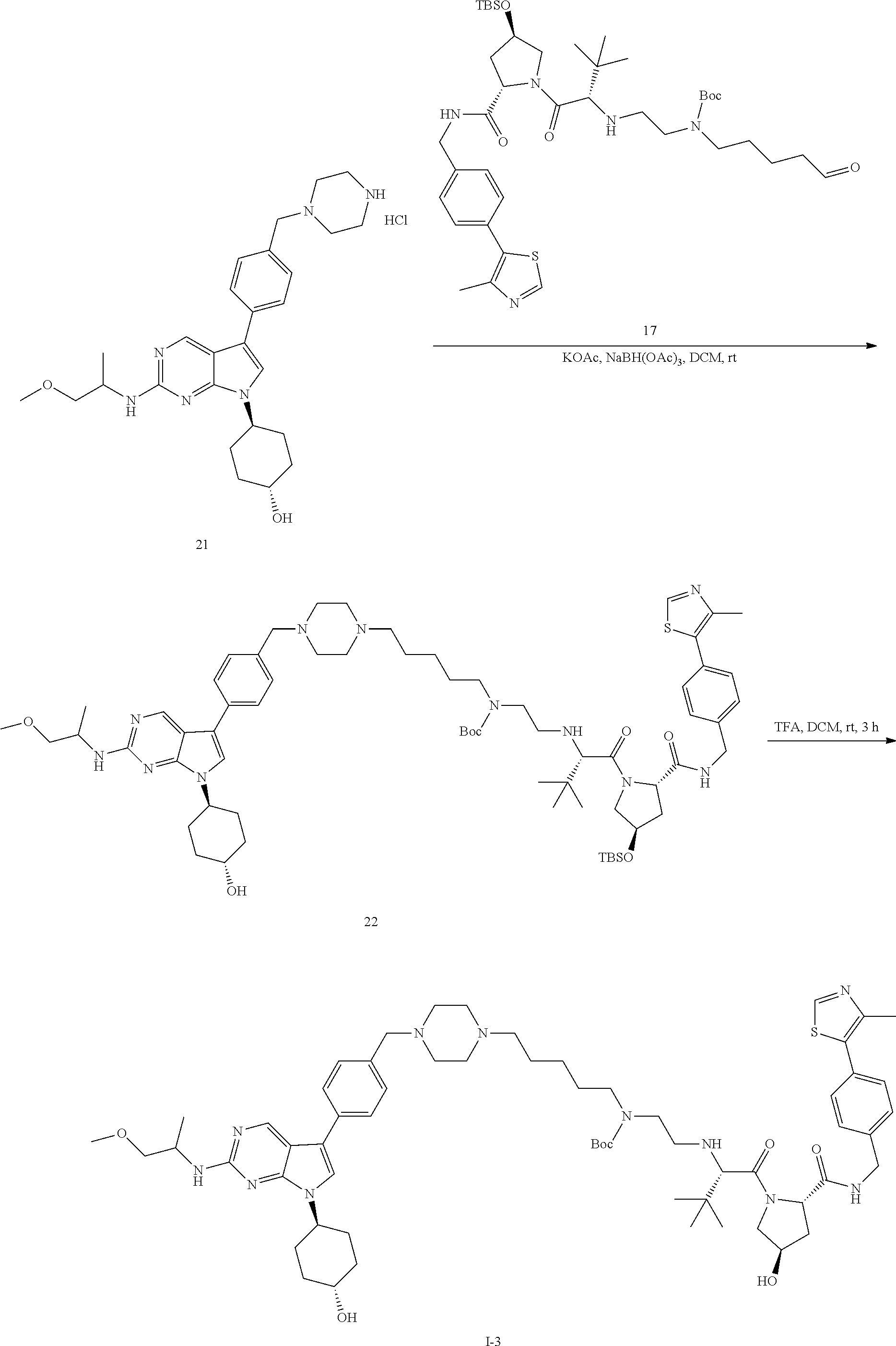

### Step 1: 5-Bromopentyl benzoate

To a stirred solution of 5-bromopentan-1-ol (10 g, 60.2 mmol) in DCM (100 mL) were added pyridine (7.1 g, 90 mmol) and benzoyl chloride (10.2 g, 72.5 mmol) at 0° C. The resulting mixture was stirred for 3 h at room temperature under nitrogen atmosphere. The resulting mixture was concentrated under reduced pressure. The residue was purified by silica gel column chromatography, eluted with 1% ethyl acetate in petroleum ether to afford 5-bromopentyl benzoate (12.5 g, 77%) as a colorless oil. ¹H NMR (400 MHz, CDCl₃) δ 8.07 (dd, J=8.4, 1.5 Hz, 2H), 7.62-7.53 (m, 1H), 7.46 (dd, J=8.4, 7.0 Hz, 2H), 4.36 (t, J=6.5 Hz, 2H), 3.46 (t, J=6.7 Hz, 2H), 1.96 (p, J=7.0 Hz,2H), 1.82 (dt, J=8.3, 6.5 Hz, 2H), 1.64 (dddd, J=14.8, 9.5, 6.6, 3.5 Hz, 2H)

### Step 2: 5-[(2-Hydroxyethyl)amino]pentyl benzoate

To a solution of 5-bromopentyl benzoate (11.5 g, 42.4 mmol) in THF (220 mL) was added 2-aminoethan-1-ol (25.9 g, 424.1 mmol). The resulting solution was stirred for 3 h at 80° C. under nitrogen atmosphere. After cooling to ambient temperature, the resulting mixture was diluted with H₂O (250 mL) and extracted with EtOAc (3×250 mL). The combined organic layers was washed with brine (250 mL) and dried over anhydrous Na₂SO₄. After filtration, the filtrate was concentrated under reduced pressure. The residue was purified by silica gel column chromatography, eluted with 10% methanol in dichloromethane to afford 5-[(2-hydroxyethyl) amino] pentyl benzoate (9.2 g, 87%) as a colorless oil. ¹H NMR (400 MHz, CDCl₃) δ 8.08-8.02 (m, 2H), 7.59-7.53 (m, 1H), 7.44 (dd, J=8.4, 7.0 Hz, 2H), 4.33 (t, J=6.6 Hz, 2H), 3.66 (dd, J=5.8, 4.6 Hz, 2H), 2.81-2.74 (m, 2H), 2.66 (t, J=7.1 Hz, 2H), 2.59-2.50 (m, 2H), 1.80 (p, J=6.8 Hz, 2H), 1.63-1.56 (m, 2H), 1.50 (td, J=9.0, 8.2, 5.5 Hz, 2H). LC/MS (ESI, m/z): [(M+1)]⁺=252.25

### Step 3: 5-[[(Tert-butoxy)carbony](2-hydroxyethyl)amino]entyl benzoate

To a stirred solution of 5-[(2-hydroxyethyl)amino]pentyl benzoate (9.2 g, 36.6 mmol) in THF (200 mL) were added di-tert-butyl dicarbonate (8.4 g, 38.4 mmol) at room temperature under nitrogen atmosphere. After stirring for additional 16 h, the resulting mixture was diluted with water (200 mL) and extracted with EtOAc (3×250 mL). The combined organic layers was washed with brine (500 mL) and dried over anhydrous Na₂SO₄. After filtration, the filtrate was concentrated under reduced pressure to afford 5-[[(tert-butoxy)carbonyl](2-hydroxyethyl)amino]pentyl benzoate (12.3 g, 96%) as a colorless oil. ¹H NMR (400 MHz, CDCl₃) δ 8.04-7.98 (m, 2H), 7.53 (t, J=7.5 Hz, 1H), 7.42 (d, J=7.6 Hz, 2H), 4.30 (t, J=6.5 Hz, 2H), 3.70 (t, J=5.5 Hz, 2H), 3.35 (s, 2H), 3.28-3.18 (m, 2H), 1.76 (d, J=7.5 Hz, 2H), 1.59 (d, J=8.0 Hz, 2H), 1.50-1.38 (m, 11H). LC/MS (ESI, m/z): [(M+1-100)]⁺=252.30

### Step 4: 5-((tert-butoxycarbonyl)(2-oxoethyl)amino)pentyl benzoate

To a stirred solution of 5-[[(tert-butoxy)carbonyl](2-hydroxyethyl)amino]pentyl benzoate (1 g, 2.84 mmol) in DCM (20 mL) was added Dess-Martin reagent (1.8 g, 4.27 mmol) at room temperature. The resulting mixture was stirred for 2 h at room temperature under nitrogen atmosphere. The resulting mixture was concentrated under reduced pressure. The residue was purified by reverse phase flash chromatography with the following conditions: Column: WelFlash™ C18-I, 20-40 um, 120 g; Eluent A: Water (plus 10 mmol/L HOAc); Eluent B: ACN; Gradient: 50%-70% B in 25 min; Flow rate: 50 mL/min; Detector: 220/254 nm; desired fractions were collected at 55% B and concentrated under reduced pressure to afford 5-[[(tert-butoxy)carbonyl](2-oxoethyl)amino]pentyl benzoate (0.4 g, 40%) as a colorless oil. ¹H NMR (400 MHz, CDCl₃) δ 9.59 (d, J=5.2 Hz, 1H), 8.08-8.03 (m, 2H), 7.61-7.55 (m, 1H), 7.46 (dd, J=8.4, 7.0 Hz, 2H), 4.34 (q, J=5.8 Hz, 2H), 4.00-3.80 (m, 2H), 3.40-3.27 (m, 2H), 1.82 (p, J=6.8 Hz, 2H), 1.65-1.57 (m, 2H), 1.52-1.41 (m, 11H). LC/MS (ESI, m/z): [(M+1)]⁺=350.30

### Step 5: Tert-butyl N-[[4-(4-methyl-1,3-thiazol-5-yl)phenyl]methyl]carbamate

To a solution of 5-bromo-4-methyl-1,3-thiazole (1.9 g, 10.7 mmol) in dioxane (19 mL) were added H₂O (1.9 mL), [4-([[(tert-butoxy)carbonyl]amino]methyl)phenyl]boronic acid (4.0 g, 16.0 mmol), Na₂CO₃ (2.3 g, 21.3 mmol) and Pd(dppf)Cl₂.CH₂Cl₂ (871.4 mg, 1.1 mmol). The mixture was purged with N₂ for 3 times and was stirred at 90° C. for 2 hours under N₂ atmosphere. The resulting mixture was cooled to room temperature, diluted with water (50 mL) and extracted with ethyl acetate (3×50 mL). The combined organic layers was washed with brine (2×30 mL) and dried over anhydrous sodium sulfate. After filtration, the filtrate was concentrated under reduced pressure. The residue was purified by silica gel column chromatography, eluted with petroleum ether in ethyl acetate (v/v=1/1) to afford tert-butyl N-[[4-(4-methyl-1,3-thiazol-5-yl)phenyl]methyl]carbamate (3 g, 91%) as a yellow solid. ¹H NMR (400 MHz, DMSO-d₆) δ 8.98 (s, 1H), 7.53-7.42 (m, 3H), 7.33 (d, J=8.0 Hz, 2H), 4.16 (d, J=6.2 Hz, 2H), 2.45 (s, 3H), 1.40 (s, 9H). LC/MS (ESI, m/z): [(M+1)]⁺=305.2

### Step 6: [4-(4-Methyl-1,3-thiazol-5-yl)pheny]methanamine hydrochloride

To a solution of tert-butyl N-[[4-(4-methyl-1,3-thiazol-5-yl)phenyl]methyl]carbamate (2.9 g, 9.53 mmol) in dioxane (20 mL) was added a solution of HCl in dioxane (4 M, 20 mL). The mixture was stirred at room temperature for 30 min. The resulting mixture was concentrated under vacuum to afford [4-(4-methyl-1,3-thiazol-5-yl)phenyl]methanamine hydrochloride (2.36 g, 99%) as a yellow solid. ¹H NMR (400 MHz, DMSO-d₆) δ 9.12 (s, 1H), 8.59 (br s, 3H), 7.62 (d, J=8.0 Hz, 2H), 7.55 (d, J=8.1 Hz, 2H), 4.06 (q, J=5.9 Hz, 2H), 2.47 (s, 3H). LC/MS (ESI, m/z): [(M+1)]⁺=205.2

### Step 7: Tert-butyl (2S,4R)-4-hydroxy-2-([[4-(4-methyl-1,3-thiazol-5-yl)phenyl]methyl]carbamoyl)pyrrolidine-1-carboxylate

To a solution of (2S,4R)-1-[(tert-butoxy)carbonyl]-4-hydroxypyrrolidine-2-carboxylic acid (2.81 g, 12.15 mmol) in DCM (56 mL) were added [4-(4-methyl-1,3-thiazol-5-yl)phenyl]methanamine hydrochloride (2.66 g, 11.05 mmol), HATU (5.04 g, 13.26 mmol) and TEA (3.35 g, 33.15 mmol). The mixture was stirred at room temperature for 2 hours. The resulting mixture was diluted with water (50 mL) and extracted with dichloromethane (3×50 mL). The combined organic layers was washed with brine (2×30 mL) and dried over anhydrous sodium sulfate. After filtration, the filtrate was concentrated under reduced pressure. The residue was purified by silica gel column chromatography, eluted with methanol in dichloromethane (v/v=10/1) to afford tert-butyl (2S,4R)-4-hydroxy-2-([[4-(4-methyl-1,3-thiazol-5-yl)phenyl]methyl]carbamoyl)pyrrolidine-1-carboxylate (3.8 g, 82%) as a yellow solid. ¹H NMR (400 MHz, DMSO-d₆) δ 8.99 (s, 1H), 8.48 (dt, J=10.7, 5.9 Hz, 1H), 7.50-7.19 (m, 4H), 4.46-4.10 (m, 4H), 3.30 (s, 2H), 2.44 (s, 3H), 2.08 (t, J=10.9 Hz, 1H), 1.85 (ddd, J=12.8, 8.2, 4.8 Hz, 1H), 1.25 (s, 9H). LC/MS (ESI, m/z): [(M+1)]⁺=418.2

### Step 8: (2S,4R)-4-hydroxy-N-[[4-(4-methyl-1,3-thiazol-5-yl)phenyl]methyl]pyrrolidine-2-carboxamide hydrochloride

To a solution of tert-butyl (2S,4R)-4-hydroxy-2-([[4-(4-methyl-1,3-thiazol-5-yl)phenyl]methyl]carbamoyl)pyrrolidine-1-carboxylate (3.7 g, 8.86 mmol) in dioxane (35 mL) was added a solution of HCl in dioxane (4 M, 35 mL). The mixture was stirred at room temperature for 2 hours. The resulting mixture was concentrated under reduced pressure to afford (2S,4R)-4-hydroxy-N-[[4-(4-methyl-1,3-thiazol-5-yl)phenyl]methyl]pyrrolidine-2-carboxamide hydrochloride (3.2 g, 99%) as yellow solid. ¹H NMR (400 MHz, CD₃OD) δ 9.96 (s, 1H), 7.60 (d, J=8.3 Hz, 2H), 7.55 (d, J=8.3 Hz, 2H), 4.63 (t, J=3.8 Hz, 1H), 4.61-4.50 (m, 3H), 3.80-3.66 (m, 1H), 3.46 (dd, J=12.1, 3.6 Hz, 1H), 2.63 (s, 3H), 2.53 (ddt, J=13.4, 7.5, 1.7 Hz, 1H), 2.11 (ddd, J=13.3, 10.6, 4.0 Hz, 1H). LC/MS (ESI, m/z): [(M+1)]⁺=318.0

### Step 9: Tert-butyl N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamate

To a solution of (2S)-2-[[(tert-butoxy)carbonyl]amino]-3,3-dimethylbutanoic acid (2.23 g, 9.64 mmol) in DMF (40 mL) were added HATU (4.0 g, 10.51 mmol), TEA (2.66 g, 26.28 mmol) and (2S,4R)-4-hydroxy-N-[[4-(4-methyl-1,3-thiazol-5-yl)phenyl]methyl]pyrrolidine-2-carboxamide hydrochloride (3.1 g, 8.76 mmol). The mixture was stirred at room temperature for 2 hours. The resulting mixture was diluted with water (100 mL) and extracted with ethyl acetate (3×100 mL). The combined organic layers was washed with water (5×40 mL), brine (100 mL) and dried over anhydrous sodium sulfate. After filtration, the filtrate was concentrated under reduced pressure. The residue was purified by silica gel column chromatography, eluted with methanol in dichloromethane (v/v=1/1) to afford tert-butyl N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamate (4.6 g, 99%) as a yellow solid. ¹H NMR (400 MHz, DMSO-d₆) δ 8.98 (s, 1H), 8.57 (t, J=6.0 Hz, 1H), 7.44-7.36 (m, 4H), 6.45 (d, J=9.2 Hz, 1H), 5.14 (br s, 1H), 4.47-4.40 (m, 2H), 4.27-4.12 (m, 2H), 3.74-3.53 (m, 2H), 2.44 (s, 3H), 2.10-1.84 (m, 2H), 1.38 (s, 9H), 0.93 (s, 9H). LC/MS (ESI, m/z): [(M+1)]⁺=530.9

### Step 10: (2S,4R)-1-[(2S)-2-amino-3,3-dimethylbutanoy]-4-hydroxy-N-[[4-(4-methyl-1,3-thiazol-5-yl)phenyl]methyl]pyrrolidine-2-carboxamide hydrochloride

To a solution of tert-butyl N-[(2S)-1-[(2S,4R)-4-hydroxy-2-([[4-(4-methyl-1,3-thiazol-5-yl)phenyl]methyl]carbamoyl)pyrrolidin-1-yl]-3,3-dimethyl-1-oxobutan-2-yl]carbamate (4.5 g, 8.48 mmol) in dioxane (30 mL) was added a solution of HCl in dioxane (4 M, 30 mL). The mixture was stirred at room temperature for 2 hours. The resulting mixture was concentrated under vacuum to afford (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide hydrochloride (4.2 g, 92%) as a yellow solid. ¹H NMR (400 MHz, DMSO-d₆) δ 9.38-9.10 (m, 1H), 8.87-8.69 (m, 1H), 8.20 (br s, 3H), 7.41 (s, 4H), 4.55 (t, J=8.3 Hz, 1H), 4.48-4.32 (m, 2H), 4.24 (dd, J=15.8, 5.5 Hz, 1H), 3.88 (d, J=5.3 Hz, 1H), 3.80 (d, J=11.0 Hz, 1H), 3.60-3.50 (m, 1H), 2.46 (s, 3H), 2.12 (dd, J=12.8, 7.9 Hz, 1H), 1.87 (ddd, J=13.1, 9.1, 4.3 Hz, 1H), 1.02 (s, 9H). LC/MS (ESI, m/z): [(M+1)]⁺=431.1

### Step 11: (2S,4R)-1-[(2S)-2-amino-3,3-dimethylbutanoyl]-4-[(tert-butyldimethylsilyl)oxy]-N-[[4-(4-methyl-1,3-thiazol-5-yl)phenyl]methyl]pyrrolidine-2-carboxamide

To a solution of (2S,4R)-1-[(2S)-2-amino-3,3-dimethylbutanoyl]-4-hydroxy-N-[[4-(4-methyl-1,3-thiazol-5-yl)phenyl]methyl]pyrrolidine-2-carboxamide hydrochloride (2 g, 4.28 mmol) in DCM (40 mL) were added imidazole (1.46 g, 21.45 mmol) and TBS-Cl (0.97 g, 6.42 mmol) in portions at 0° C. The resulting mixture was stirred at room temperature for 16 h under nitrogen atmosphere. The reaction was quenched with water (40 mL) and extracted with DCM (3×50 mL). The combined organic layers was washed with brine (50 mL) and dried over anhydrous Na₂SO₄. After filtration, the filtrate was concentrated under reduced pressure. The residue was purified by silica gel column chromatography, eluted with EtOAc to afford (2S,4R)-1-[(2S)-2-amino-3,3-dimethylbutanoyl]-4-[(tert-butyl dimethylsilyl)oxy]-N-[[4-(4-methyl-1,3-thiazol-5-yl)phenyl]methyl]pyrrolidine-2-carboxamide (1.4 g, 60%) as a green solid. ¹H NMR (400 MHz, CDCl₃) δ 8.70 (s, 1H), 7.53 (s, 1H), 7.45-7.31 (m, 4H), 4.78 (dd, J=8.3, 4.5 Hz, 1H), 4.64 (p, J=5.4 Hz, 1H), 4.53 (dd, J=14.8, 6.4 Hz, 1H), 4.38 (dd, J=14.8, 5.3 Hz, 1H), 3.66 (dd, J=10.2, 5.6 Hz, 1H), 3.49 (dd, J=10.3, 4.7 Hz, 1H), 3.29 (s, 1H), 2.61 (dt, J=10.6, 4.9 Hz, 1H), 2.54 (s, 3H), 1.92 (ddd, J=13.3, 8.3, 5.8 Hz, 1H), 0.92 (s, 9H), 0.90 (s, 9H), 0.12 (s, 3H), 0.11 (s, 3H). LC/MS (ESI, m/z): [(M+1)]⁺=545.40

### Step 12: 5-[[(Tert-butoxy)carbonyl]-(2-[[tert-butoxy)carbonyl][(2S)-1-[(2S,4R)-4-[(tert-butyldimethylsily)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)amino]entyl benzoate

To a stirred solution of (2S,4R)-1-[(2S)-2-amino-3,3-dimethylbutanoyl]-4-[(tert-butyldimethylsilyl)oxy]-N-[[4-(4-methyl-1,3-thiazol-5-yl)phenyl]methyl]pyrrolidine-2-carboxamide (519 mg, 0.95 mmol) in DCM (4 mL) were added 5-[[(tert-butoxy)carbonyl](2-oxoethyl)amino]pentyl benzoate (400 mg, 1.14 mmol), HOAc (0.26 mL, 4.32 mmol) and NaBH(AcO)₃ (606 mg, 2.86 mmol). The resulting mixture was stirred at room temperature for 2 h. The resulting mixture was concentrated under vacuum. The residue was purified by reverse phase flash chromatography with the following conditions: Column: WelFlash™ C18-I, 20-40 um, 120 g; Eluent A: Water (plus 10 mmol/L HOAc); Eluent B: ACN; Gradient: 80%-99% B in 25 min; Flow rate: 50 mL/min; Detector: 220/254 nm; desired fractions were collected at 91% B and concentrated under reduced pressure to afford 5-[[(tert-butoxy)carbonyl](2-[[(tert-butoxy)carbonyl][(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)amino]pentyl benzoate (409.7 mg, 44%) as a green oil. ¹H NMR (400 MHz, CDCl₃) δ 8.70 (s, 1H), 8.04 (d, J=7.6 Hz, 2H), 7.58 (dd, J=17.2, 9.7 Hz, 2H), 7.45 (t, J=7.6 Hz, 2H), 7.36 (t, J=6.3 Hz, 4H), 4.85-4.74 (m, 1H), 4.61-4.47 (m, 2H), 4.44-4.27 (m, 3H), 3.59 (d, J=4.8 Hz, 1H), 3.51 (s, 2H), 3.40-3.13 (m, 4H), 3.11-2.97 (m, 1H), 2.67 (dd, J=11.9, 6.1 Hz, 1H), 2.53 (s, 3H), 2.50-2.32 (m, 1H), 2.02-1.90 (m, 1H), 1.80 (t, J=7.4 Hz, 2H), 1.66-1.52 (m, 2H), 1.50-1.38 (m, 8H), 1.35-1.21 (m, 3H), 0.89 (s, 18H), 0.11 (s, 3H), 0.09 (s, 3H). LC/MS (ESI, m/z): [(M+1)]⁺=878.6

### Step 13: Tert-butyl N-(2-[[(tert-butoxy)carbonyl][(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)-N-(5-hydroxypentyl)carbamate

To a stirred solution of 5-[[(tert-butoxy)carbonyl](2-[[(tert-butoxy)carbonyl][(2S)-1-[(2S,4R)-4-[(tert-butyldimethylsilyl)oxy]-2-([[4-(4-methyl-1,3-thiazol-5-yl)phenyl]methyl] carbamoyl)pyrrolidin-1-yl]-3,3-dimethyl-1-oxobutan-2-yl]amino]ethyl)amino]pentyl benzoate (409 mg, 0.42 mmol) in MeOH (8 mL) was added K₂CO₃ (173.3 mg, 1.25 mmol) and stirred at room temperature for overnight under nitrogen atmosphere. The resulting mixture was concentrated under vacuum. The residue was purified by reverse phase flash chromatography with the following conditions: Column: WelFlash™ C18-I, 20-40 um, 120 g; Eluent A: Water (plus 10 mmol/L HOAc); Eluent B: ACN; Gradient: 40%-60% B in 25 min; Flow rate: 50 mL/min; Detector: 220/254 nm; desired fractions were collected at 55% B and concentrated under reduced pressure to afford tert-butyl N-(2-[[(tert-butoxy)carbonyl][(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl] amino]ethyl)-N-(5-hydroxypentyl)carbamate (207.8 mg, 57%) as a green oil. ¹H NMR (400 MHz, CDCl₃) δ 8.90 (s, 1H), 7.49 (d, J=8.3 Hz, 2H), 7.44 (d, J=8.3 Hz, 2H), 4.75-4.59 (m, 2H), 4.56 (d, J=15.5 Hz, 1H), 4.38 (d, J=15.5 Hz, 1H), 3.86-3.68 (m, 2H), 3.57 (t, J=6.6 Hz, 2H), 3.41-3.35 (m, 1H), 3.22 (t, J=7.5 Hz, 2H), 2.67 (d, J=10.6 Hz, 1H), 2.61-2.55 (m, 1H), 2.49 (s, 3H), 2.22 (dd, J=13.1, 7.6 Hz, 1H), 2.15-2.05 (m, 1H), 2.02-1.96 (m, 1H), 1.63-1.51 (m, 4H), 1.48 (s, 10H), 1.41-1.26 (m, 2H), 1.02 (s, 9H), 0.91 (s, 9H), 0.15 (s, 3H), 0.13 (s, 3H). LC/MS (ESI, m/z): [(M+1)]⁺=774.45

### Step 14: Tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)-N-(5-oxopentyl)carbamate

To a stirred solution of tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)-N-(5-hydroxypentyl)carbamate (100 mg, 0.13 mmol) in DCM (2 mL) was added Dess-Martin reagent (71.22 mg, 0.17 mmol) at room temperature. The resulting mixture was stirred for 2 h at room temperature under nitrogen atmosphere. The resulting mixture was diluted with water (10 mL) and extracted with CH₂Cl₂ (2×20 mL). The combined organic layers was washed with saturated NaHCO₃ (aq.) (20 mL) and dried over anhydrous Na₂SO₄. After filtration, the filtrate was concentrated under reduced pressure to afford tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino] ethyl)-N-(5-oxopentyl)carbamate (100 mg, crude) as a yellow solid. LC/MS (ESI, m/z): [(M+1)]⁺=772.50.

### Step 15: 4-[5-Bromo-2-[(1-methoxypropan-2-yl)amino]-7H-pyrrolo[2,3-d]pyrimidin-7-yl]cyclohexan-1-ol

To a solution of 4-[5-bromo-2-chloro-7H-pyrrolo[2,3-d]pyrimidin-7-yl]cyclohexan-1-ol (600 mg, 1.82 mmol) in NMP (12 mL) were added 1-methoxypropan-2-amine (485 mg, 5.44 mmol) and DIEA (469 mg, 3.63 mmol). The resulting mixture was stirred for overnight at 110° C. under nitrogen atmosphere. After cooling down to room temperature, the resulting solution was purified by reverse phase flash chromatography with the following conditions: Column: WelFlash™ C₁₈-I, 20-40 μm, 330 g; Eluent A: Water (plus 10 mmol/L FA); Eluent B: ACN; Gradient: 25%-45% B in 25 min; Flow rate: 80 mL/min; Detector: 220/254 nm; desired fractions were collected at 34% B and concentrated under reduced pressure to afford 4-[5-bromo-2-[(1-methoxypropan-2-yl)amino]-7H-pyrrolo[2,3-d]pyrimidin-7-yl]cyclohexan-1-ol (400 mg, 58%) as a brown solid. ¹H NMR (400 MHz, DMSO-d₆) δ 8.37 (s, 1H), 7.39 (s, 1H), 6.79 (d, J=8.0 Hz, 1H), 4.37 (tt, J=11.1, 4.5 Hz, 1H), 4.18 (p, J=6.5 Hz, 1H), 3.47 (m, 2H), 3.28 (s, 3H), 3.27-3.21 (m, 1H), 1.98-1.82 (m, 6H), 1.32 (qd, J=12.8, 6.4 Hz, 2H), 1.15 (d, J=6.7 Hz, 3H). LC/MS (ESI, m/z): [(M+1)]⁺=383.20, 385.20

### Step 16: Tert-butyl 4-([4-[7-(4-hydroxycyclohexyl)-2-[(1-methoxypropan-2-yl)amino]-7H-pyrrolo[2,3-d]pyrimidin-5-yl]phenyl]methyl)piperazine-1-carboxylate

To a stirred solution of 4-[5-bromo-2-[(1-methoxypropan-2-yl)amino]-7H-pyrrolo[2,3-d]pyrimidin-7-yl]cyclohexan-1-ol (450 mg, 1.17 mmol) in dioxane (12 mL) were added H₂O (4 mL), tert-butyl 4-[[4-(4,4,5,5-tetramethyl-1,3,2-dioxaborolan-2-yl)phenyl]methyl]piperazine-1-carboxylate (708 mg, 1.76 mmol), K₂CO₃ (487 mg, 3.52 mmol) and Pd(PPh₃)₄ (67.83 mg, 0.059 mmol) at room temperature. The resulting mixture was stirred for 4 h at 100° C. under nitrogen atmosphere. The mixture was cooled down to room temperature. The resulting mixture was diluted with water (10 mL) and extracted with EtOAc (3×10 mL). The combined organic layers were washed with brine (20 mL) and dried over anhydrous Na₂SO₄. After filtration, the filtrate was concentrated under reduced pressure. The residue was purified by reverse phase flash chromatography with the following conditions: Column: WelFlash™ C₁₈-I, 20-40 μm, 330 g; Eluent A: Water (plus 10 mmol/L FA); Eluent B: ACN; Gradient: 60%-80% B in 25 min; Flow rate: 80 mL/min; Detector: 220/254 nm; desired fractions were collected at 72% B and concentrated under reduced pressure to afford tert-butyl 4-([4-[7-(4-hydroxycyclohexyl)-2-[(1-methoxypropan-2-yl)amino]-7H-pyrrolo [2,3-d]pyrimidin-5-yl]phenyl]methyl)piperazine-1-carboxylate (360 mg, 53%) as a yellow oil. ¹H NMR (400 MHz, DMSO-d₆) δ 8.84 (s, 1H), 7.68-7.58 (m, 3H), 7.32 (d, J=8.0 Hz, 2H), 6.63 (d, J=8.1 Hz, 1H), 4.71 (d, J=4.4 Hz, 1H), 4.42 (q, J=11.0, 8.9 Hz, 1H), 4.21 (p, J=6.7 Hz, 1H), 3.60-3.43 (m, 4H), 3.33-3.31 (m, 4H), 3.30 (s, 3H), 3.26 (dd, J=9.3, 6.8 Hz, 1H), 2.38-2.28 (m, 4H), 2.05-1.84 (m, 6H), 1.39 (s, 9H), 1.39-1.28 (m, 2H), 1.17 (d, J=6.6 Hz, 3H). LC/MS (ESI, m/z): [(M+1)]⁺=579.40

### Step 17: 4-[2-[(1-Methoxypropan-2-yl)amino]-5-[4-[(piperazin-1-yl)methyl]phenyl]-7H-pyrrolo[2,3-d]pyrimidin-7-yl]cyclohexan-1-ol hydrochloride

To a stirred solution of tert-butyl 4-([4-[7-(4-hydroxycyclohexyl)-2-[(1-methoxypropan-2-yl)amino]-7H-pyrrolo [2,3-d]pyrimidin-5-yl]phenyl]methyl)piperazine-1-carboxylate (360 mg, 0.62 mmol) in dioxane (5 mL) was added a solution of HCl in dioxane (4 M, 5 mL) dropwise at room temperature under nitrogen atmosphere. The resulting mixture was stirred for 2 h at room temperature under nitrogen atmosphere. The resulting mixture was concentrated under reduced pressure to afford 4-[2-[(1-methoxypropan-2-yl)amino]-5-[4-[(piperazin-1-yl)methyl]phenyl]-7H-pyrrolo[2,3-d]pyrimidin-7-yl]cyclohexan-1-ol hydrochloride (300 mg, 94%) as a yellow solid. ¹H NMR (400 MHz, CD₃OD) δ 8.88 (s, 1H), 8.01 (s, 1H), 7.83 (d, J=7.9 Hz, 2H), 7.77 (d, J=8.0 Hz, 2H), 4.64 (p, J=8.1 Hz, 1H), 4.54 (s, 2H), 4.42 (d, J=6.6 Hz, 1H), 3.82-3.52 (m, 11H), 3.44 (s, 3H), 2.27-1.94 (m, 6H), 1.57 (d, J=10.0 Hz, 2H), 1.38 (d, J=6.7 Hz, 3H). LC/MS (ESI, m/z): [(M+1)]⁺=479.30

### Step 18: Tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)-N-(5-[4-[(4-[2-[(1-methoxypropan-2-yl)amino]-7-[trans-4-hydroxycyclohexyl]-7H-pyrrolo[2,3-d]pyrimidin-5-yl]phenyl)methyl]piperazin-1-yl]pentyl)carbamate

To a stirred solution of 4-[2-[(1-methoxypropan-2-yl)amino]-5-[4-[(piperazin-1-yl)methyl]phenyl]-7H-pyrrolo[2,3-d]pyrimidin-7-yl]cyclohexan-1-ol hydrochloride (100 mg, 0.194 mmol) in DCM (4 mL) were added KOAc (76.21 mg, 0.78 mmol), tert-butyl N-(2-[[(2S)-1-[(2S,4R)-4-[(tert-butyldimethylsilyl)oxy]-2-([[4-(4-methyl-1,3-thiazol-5-yl)phenyl]methyl] carbamoyl)pyrrolidin-1-yl]-3,3-dimethyl-1-oxobutan-2-yl]amino]ethyl)-N-(5-oxopentyl) carbamate (149.90 mg, 0.19 mmol) and NaBH(AcO)₃ (164.58 mg, 0.78 mmol). The resulting mixture was stirred for overnight at room temperature under nitrogen atmosphere. The resulting mixture was concentrated under reduced pressure. The residue was purified by Prep-HPLC with the following conditions (Column: Xselect CSH OBD Column 30×150 mm 5 um; Mobile Phase A: Water (plus 10 mmol/L FA), Mobile Phase B: ACN; Flow rate: 20 mL/min; Gradient: 8% B to 35% B in 8 min; Detector: 254/220 nm; Rt: 8.53 min) to afford tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino]ethyl)-N-(5-[4-[(4-[2-[(1-methoxypropan-2-yl)amino]-7-[trans-4-hydroxycyclohexyl]-7H-pyrrolo[2,3-d]pyrimidin-5-yl]phenyl)methyl] piperazin-1-yl]pentyl)carbamate (80 mg, 33%) as a yellow solid. LC/MS (ESI, m/z): [(M/2+1)]⁺=618.30

### Step 19: (2S,4R)-4-Hydroxy-1-[(2S)-2-([2-[(5-[4-[(4-[2-[(1-methoxypropan-2-yl)amino]-7-[trans-4-hydroxycyclohexyl]-7H-pyrrolo[2,3-d]pyrimidin-5-yl]phenyl)methyl]piperazin-1-yl]pentyl)amino]ethyl]amino)-3,3-dimethylbutanoyl]-N-[[4-(4-methyl-1,3-thiazol-5-yl)phenyl]methyl]pyrrolidine-2-carboxamide

To a stirred solution of tert-butyl N-(2-[[(2S)-1-[(2S,4R)-4-[(tert-butyldimethylsilyl)oxy]-2-([[4-(4-methyl-1,3-thiazol-5-yl)phenyl]methyl]carbamoyl)pyrrolidin-1-yl]-3,3-dimethyl-1-oxobutan-2-yl]amino] ethyl)-N-(5-[4-[(4-[2-[(1-methoxypropan-2-yl)amino]-7-[trans-4-hydroxycyclohexyl]-7H-pyrrolo[2,3-d]pyrimidin-5-yl]phenyl)methyl]piperazin-1-yl]pentyl)carbamate (50 mg, 0.04 mmol) in DCM (2 mL) was added TFA (0.5 mL, 6.73 mmol) dropwise at room temperature under nitrogen atmosphere. The resulting solution was stirred for 3 h at room temperature under nitrogen atmosphere. The resulting solution was concentrated under reduced pressure. The residue was purified by Prep-HPLC with the following conditions (Column: Atlantis HILIC OBD Column 19×150 mm 5 um; Mobile Phase A: Water (plus 10 mmol/L FA); Mobile Phase B: ACN; Flow rate: 60 mL/min; Gradient: 22% B to 38% B in 7 min; Detector: 254/220 nm; Rt: 6.66 min) to afford (2S,4R)-4-hydroxy-1-[(2S)-2-([2-[(5-[4-[(4-[2-[(1-methoxypropan-2-yl)amino]-7-[trans-4-hydroxycyclohexyl]-7H-pyrrolo[2,3-d]pyrimidin-5-yl]phenyl)methyl]piperazin-1-yl]pentyl)amino]ethyl]amino)-3,3-dimethylbutanoyl]-N-[[4-(4-methyl-1,3-thiazol-5-yl)phenyl]methyl]pyrrolidine-2-carboxamide (15 mg, 36%) as a white solid. ¹H NMR (400 MHz, CD₃OD) δ 8.89 (s, 1H), 8.73 (s, 1H), 7.65 (d, J=7.7 Hz, 2H), 7.54-7.39 (m, 7H), 4.72-4.46 (m, 6H), 4.41-4.27 (m, 2H), 3.90-3.67 (m, 5H), 3.57 (d, J=5.3 Hz, 2H), 3.47 (d, J=6.2 Hz, 1H), 3.43 (s, 3H), 3.18-2.90 (m, 6H), 2.90-2.70 (m, 7H), 2.56-2.47 (m, 3H), 2.33-2.23 (m, 1H), 2.18-2.10 (m, 3H), 2.09-2.01 (m, 4H), 1.79-1.67 (m, 4H), 1.62-1.41 (m, 4H), 1.36-1.28 (m, 4H), 1.05 (s, 9H). LC/MS (ESI, m/z): [(M+1)]⁺=1020.65.

### EXAMPLE 38: 3-[5-(1-hydroxyethyl)-3-methyl-2-oxo-2,3-dihydro-1H-1,3-benzodiazol-1-yl]piperidine-2,6-dione (I-235)

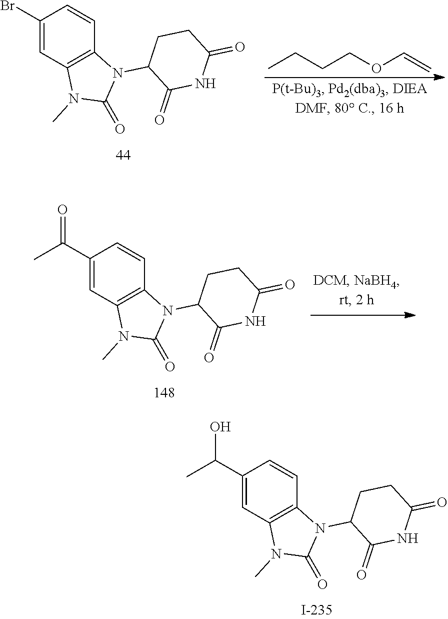

### Step 1: 3-(5-acetyl-3-methyl-2-oxo-2,3-dihydro-1H-1,3-benzodiazol-1-yl)piperidine-2,6-dione

To a stirred solution of 3-(5-bromo-3-methyl-2-oxo-2,3-dihydro-1H-1,3-benzodiazol-1-yl)piperidine-2,6-dione (1.00 g, 2.96 mmol), 1-(ethenyloxy)butane (888 mg, 8.87 mmol), DIEA (764 mg, 5.91 mmol and a solution of P(t-Bu)₃ in hexane (1.19 g, 0.591 mmol) in DMF (15.0 mL) was added Pd2(dba)3 (271 mg, 0.30 mmol) at room temperature under nitrogen atmosphere. The mixture was for 16 h at 80° C. The resulting solution was purified by reverse flash chromatography with the following conditions: Column: WelFlash™ C18-I, 20-40 μm, 330 g; Eluent A: Water (plus 10 mmol/L FA); Eluent B: ACN; Gradient: 20%-40% B in 20 min; Flow rate: 80 mL/min; Detector: UV 220/254 nm; desired fractions were collected at 32% B and concentrated under reduced pressure to afford 3-(5-acetyl-3-methyl-2-oxo-2,3-dihydrobenzo[d]imidazol-1-yl)piperidine-2,6-dione (536 mg, 60%) as an off white solid. ¹H NMR (400 MHz, DMSO-d₆) δ 11.16 (s, 1H), 7.80-7.69 (m, 2H), 7.26 (d, J=8.7 Hz, 1H), 5.46 (dd, J=12.8, 5.4 Hz, 1H), 3.42 (s, 3H), 2.98-2.85 (m, 1H), 2.76-2.63 (m, 2H), 2.60 (s, 3H), 2.05-2.03 (m, 1H). LC/MS (ESI, m/z): [(M+1)]⁺=302.25.

### Step 2: 3-[5-(1-hydroxyethyl)-3-methyl-2-oxo-2,3-dihydro-1H-1,3-benzodiazol-1-yl]piperidine-2,6-dione

To a stirred solution of 3-(5-acetyl-3-methyl-2-oxo-2,3-dihydro-1H-1,3-benzodiazol-1-yl)piperidine-2,6-dione (50.0 mg, 0.17 mmol) in DCM (10.0 mL) was added NaBH₄ (9.42 mg, 0.25 mmol) at room temperature under nitrogen atmosphere. The solution was stirred for 2 h at room temperature. The reaction was quenched with water (20.0 mL) and extracted with DCM (3×15.0 mL). The combined organic layers were dried over anhydrous Na₂SO₄. After filtration, the filtrate was concentrated under reduced pressure. The residue was purified by Prep-TLC (MeOH:DCM=1:10) to afford 3-[5-(1-hydroxyethyl)-3-methyl-2-oxo-2,3-dihydro-1H-1,3-benzodiazol-1-yl]piperidine-2,6-dione (15.0 mg, 30%) as a white solid. ¹HNMR (400 MHz, CD₃OD) δ 7.23 (d, J=1.5 Hz, 1H), 7.15 (dd, J=8.2, 1.5 Hz, 1H), 7.08 (d, J=8.1 Hz, 1H), 5.42-5.30 (m, 1H), 4.93 (q, J=6.5 Hz, 1H), 3.46 (s, 3H), 3.02-2.89 (m, 1H), 2.89-2.74 (m, 2H), 2.24-2.14 (m, 1H), 1.49 (d, J=6.5 Hz, 3H). LC/MS (ESI, m/z): [(M+1)]⁺=304.25.

### EXAMPLE 39: Trans-4-(5-[4-[(4-methylpiperazin-1-yl)methyl]phenyl]-2-[(3,3,3-trifluoropropyl)amino]pyrrolo[2,3-d]pyrimidin-7-yl)cyclohexan-1-ol; formic acid (I-148)

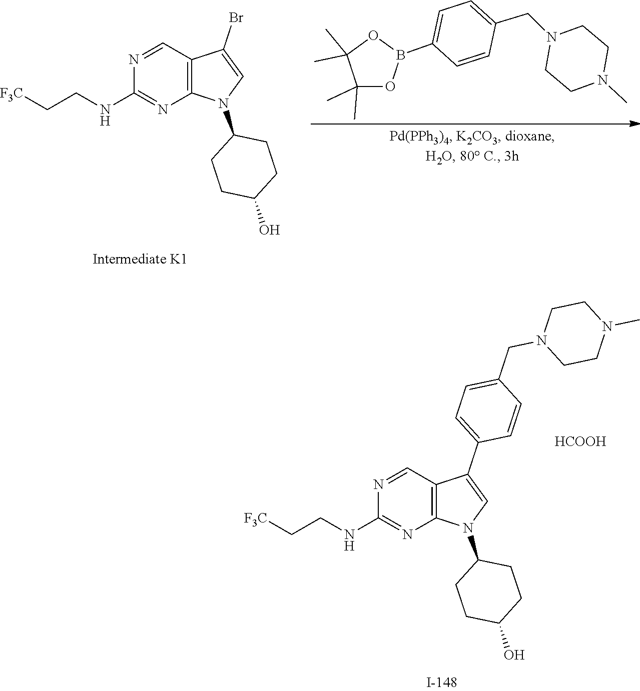

To a stirred solution of trans-4-[5-bromo-2-[(3,3,3-trifluoropropyl)amino]pyrrolo[2,3-d]pyrimidin-7-yl]cyclohexan-1-ol (100 mg, 0.25 mmol) and 1-methyl-4-[[4-(4,4,5,5-tetramethyl-1,3,2-dioxaborolan-2-yl)phenyl]methyl]piperazine (101 mg, 0.32 mmol) in 1,4-dioxane (3.00 mL) and H₂O (1.00 mL) were added K₂CO₃ (102 mg, 0.74 mmol) and Pd(PPh₃)₄ (28.4 mg, 0.025 mmol) at room temperature. The resulting mixture was purged with nitrogen for three times and stirred for 3 h at 80° C. under nitrogen atmosphere. The resulting mixture was cooled down to room temperature and diluted with water (10.0 mL). The resulting mixture was extracted with EtOAc (3×20.0 mL). The combined organic layers were washed with brine (30.0 mL) and dried over anhydrous Na₂SO₄. After filtration, the filtrate was concentrated under reduced pressure. The residue was purified by Prep-HPLC with the following conditions Column: Xselect CSH OBD Column 30×150 mm 5 um; Mobile Phase A: water (plus 0.1% FA), Mobile Phase B: ACN; Flow rate: 60 mL/min; Gradient: 5% B to 20% B in 7 min; detector: UV 220/254 nm; desired fractions were collected at 6.23 min and concentrated under reduced pressure to afford (1R,4R)-4-(5-[4-[(4-methylpiperazin-1-yl)methyl]phenyl]-2-[(3,3,3-trifluoropropyl)amino]pyrrolo[2,3-d]pyrimidin-7-yl)cyclohexan-1-ol; formic acid (70.0 mg, 51%) as a yellow solid. ¹H NMR (400 MHz, CD₃OD) δ 8.75 (s, 1H), 8.38 (s, 1H), 7.65 (d, J=8.1 Hz, 2H), 7.49-7.39 (m, 3H), 4.63-4.51 (m, 1H), 3.78-3.68 (m, 5H), 3.25-3.03 (s, 4H), 2.89-2.51 (m, 9H), 2.21-2.04 (m, 6H), 1.55 (p, J=10.8 Hz, 2H). LC/MS (ESI, m/z): [(M+1)]⁺=517.25.

### EXAMEPLE 40: (4-(((4-(1-(cyclopropylmethyl)-1H-indazol-5-yl)benzyl)amino)methyl)phenyl)(1′-(4-(7-(trans-4-hydroxycyclohexyl)-2-((3,3,3-trifluoropropyl)amino)-7H-pyrrolo[2,3-d]pyrimidin-5-yl)benzyl)-[4,4′-bipiperidin]-1-yl)methanone; formic acid (I-202)

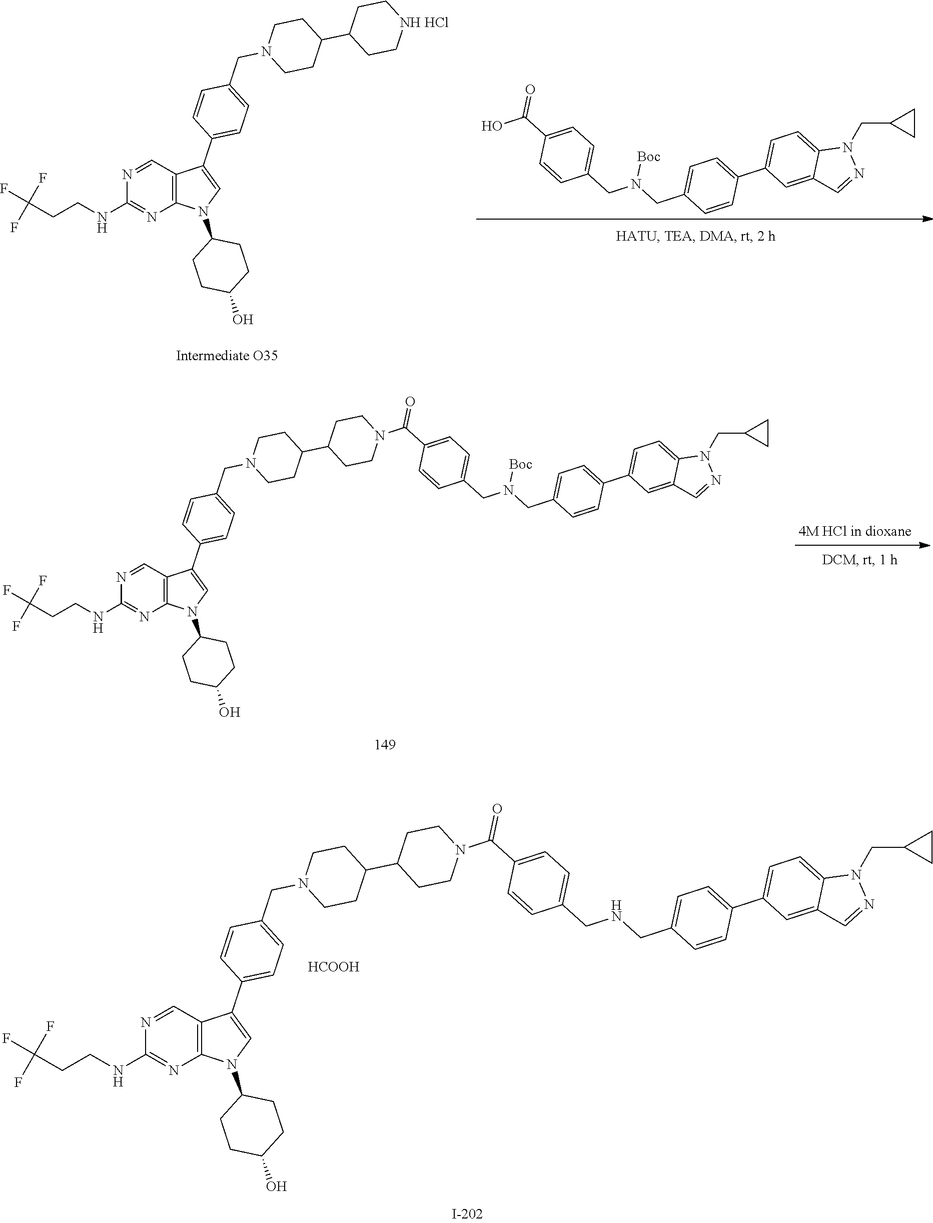

### Step 1: Tert-butyl (4-(1-(cyclopropylmethyl)-1H-indazol-5-yl)benzyl)(4-(1′-(4-(7-(trans-4-hydroxycyclohexyl)-2-((3,3,3-trifluoropropyl)amino)-7H-pyrrolo[2,3-d]pyrimidin-5-yl)benzyl)-[4,4′-bipiperidine]-1-carbonyl)benzyl)carbamate

To a stirred mixture of trans-4-[5-(4-[[4,4-bipiperidin]-1-ylmethyl]phenyl)-2-[(3,3,3-trifluoropropyl)amino]pyrrolo[2,3-d]pyrimidin-7-yl]cyclohexan-1-ol (40.0 mg, 0.068 mmol) in DMA (4.00 mL) were added TEA (32.0 mg, 0.32 mmol) and HATU (36.7 mg, 0.096 mmol) at 25° C. The resulting mixture was stirred for 10 min at 25° C. To the above mixture was added 4-[[(tert-butoxycarbonyl)([4-[1-(cyclopropylmethyl)indazol-5-yl]phenyl]methyl)amino]methyl]benzoic acid (35.4 mg, 0.069 mmol). The resulting mixture was stirred for additional 30 min at 25° C. The resulting solution was purified by reversed flash chromatography with the following conditions: Column: XBridge Prep C¹⁸ OBD Column, 5 um, 19×150 mm; Mobile Phase A: water (10 mmol/L NH₄HCO₃), Mobile Phase B:ACN; Flow rate:25 mL/min; Gradient: 50% B to 85% B in 9 min; detector: UV 220/254 nm; desired fractions were collected at 6.68 min and concentrated under reduced pressure to afford tert-butyl (4-(1-(cyclopropylmethyl)-1H-indazol-5-yl)benzyl)(4-(1′-(4-(7-(trans-4-hydroxycyclohexyl)-2-((3,3,3-trifluoropropyl)amino)-7H-pyrrolo[2,3-d]pyrimidin-5-yl)benzyl)-[4,4′-b piperidine]-1-carbonyl)benzyl)carbamate (30.0 mg, 41%) as a white solid. ¹H NMR (400 MHz, CDCl₃) δ 8.75 (s, 1H), 8.33 (s, 1H), 8.07 (d, J=1.0 Hz, 1H), 7.94 (s, 1H), 7.69-7.50 (m, 9H), 7.36 (d, J=7.9 Hz, 4H), 7.18 (s, 1H), 4.78 (s, 1H), 4.60-4.40 (m, 6H), 4.35-4.30 (m, 3H), 4.1.1 (s, 2H), 3.85-3.75 (m, 4H), 3.55-3.49 (m, 2H), 2.60-2.50 (m, 4H), 2.25-2.10 (m, 5H), 1.96-1.83 (m, 6H), 1.64 (d, 12.3 Hz, 2H), 1.54 (s, 9H), 1.53-1.35 (m, 4H), 0.68-0.59 (m, 2H), 0.47 (t, J=5.1 Hz, 2H). LC/MS (ESI, m/z): [(M+1)]⁺=1078.60.

The following examples in Table 47 were synthesized according to the above procedure:

TABLE 47

| | | | MS: | |
|---|---|---|---|---|
| Example | Structure | Name | [(M + 1)]⁺ | ¹H NMR |
| 149-1 | 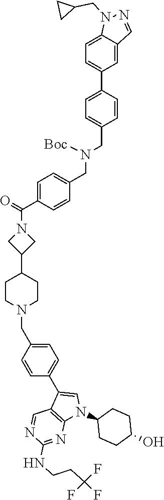 [SMILES: CC(C)(C)OC(=O)N(Cc1ccc(C(=O)N2CC(C3CCN(Cc4ccc(-c5cn([C@H]6CC[C@H](O)CC6)c6nc(NCCC(F)(F)F)ncc56)cc4)CC3)C2)cc1)Cc1ccc(-c2ccc3c(cnn3CC3CC3)c2)cc1] | tert-butyl (4-(1- (cyclopropylmethyl)- 1H-indazol-5- yl)benzyl)(4-(3-(1- (4-(7-(trans-4- hydroxycyclohexyl)- 2-((3,3,3- trifluoropropyl) amino)-7H-pyrrolo[2,3- d]pyrimidin-5- yl)benzyl)piperidin- 4-yl)azetidine-1- carbonyl)benzyl) carbamate | 1050.55 | (400 MHz, CD₃OD) δ 8.77 (s, 1H), 8.08 (s, 1H), 7.97 (s, 1H), 7.73-7.55 (m, 8H), 7.50-7.40 (m, 3H), 7.37- 7.24 (m, 4H), 4.60-4.46 (m, 6H), 4.38-4.30 (m, 3H), 4.19 (t, J = 9.4 Hz, 1H), 4.02 (t, J = 7.6 Hz, 1H), 3.92-3.83 (m, 1H), 3.79-3.68 (m, 4H), 2.60 (dt, J = 11.1, 7.3 Hz, 1H), 2.48-2.20 (m, 2H), 2.19-2.06 (m, 6H), 1.86-1.55 (m, 2H), 1.52 (s, 15H), 1.49-1.17 (m, 4H), 0.61-0.54 (m, 2H), 0.47-0.42 (m, 2H) |
| 149-2 | 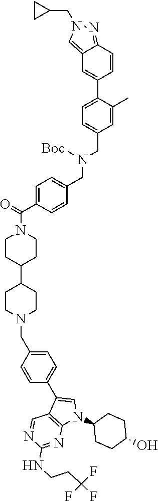 [SMILES: Cc1cc(CN(Cc2ccc(C(=O)N3CCC(C4CCN(Cc5ccc(-c6cn([C@H]7CC[C@H](O)CC7)c7nc(NCCC(F)(F)F)ncc67)cc5)CC4)CC3)cc2)C(=O)OC(C)(C)C)ccc1-c1ccc2nn(CC3CC3)cc2c1] | tert-Butyl N-([4-[2- (cyclopropylmethyl) indazol-5-yl]-3- methylphenyl] methyl)-N-[(4-[1-[(4-[7- [trans-4- hydroxycyclohexyl]- 2-[(3,3,3- trifluoropropyl) amino]pyrrolo[2,3- d]pyrimidin-5- yl]phenyl)methyl]- [4,4-bipiperidine]- 1- carbonyl]phenyl) methyl]carbamate | 1092.65 | (400 MHz, CD₃OD) δ 8.77 (s, 1H), 8.31 (d, J = 1.0 Hz, 1H), 7.68-7.56 (m, 4H), 7.46 (s, 1H), 7.40 (d, J = 7.9 Hz, 2H), 7.37-7.34 (m, 5H), 7.17 (d, J = 7.8 Hz, 1H), 7.14 (s, 1H), 7.05 (s, 1H), 4.53 (s, 4H), 4.29 (d, J = 7.2 Hz, 2H), 3.74 (d, J = 7.2 Hz, 3H), 3.70 (d, J = 12.6 Hz, 2H), 3.57 (s, 2H), 3.10-2.90 (m, 4H), 2.62-2.54 (m, 2H), 2.25 (s, 3H), 2.19-2.06 (m, 7H), 2.05 (s, 1H), 1.80-1.75 (m, 2H), 1.58-1.50 (m, 15H), 1.40-1.20 (m, 5H), 1.09 (s, 1H), 0.72-0.63 (m, 2H), 0.54-0.45 (m, 2H) |
| 149-3 | 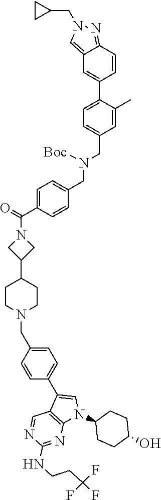 [SMILES: Cc1cc(CN(Cc2ccc(C(=O)N3CC(C4CCN(Cc5ccc(-c6cn([C@H]7CC[C@H](O)CC7)c7nc(NCCC(F)(F)F)ncc67)cc5)CC4)C3)cc2)C(=O)OC(C)(C)C)ccc1-c1ccc2nn(CC3CC3)cc2c1] | tert-butyl N-([4-[2- (cyclopropylmethyl) indazol-5-yl]-3- methylphenyl]methyl)- N-[[4-(3-[1-[(4- [7-[trans-4- hydroxycyclohexyl]- 2-[(3,3,3- trifluoropropyl) amino]pyrrolo[2,3- d]pyrimidin-5- yl]phenyl)methyl] piperidin-4- yl]azetidine-1- carbonyl)phenyl] methyl]carbamate | 1064.60 | (400 MHz, DMSO-d₆) δ 8.87 (s, 1H), 8.42 (s, 1H), 8.38 (s, 2H), 7.67-7.58 (m, 7H), 7.35-2.30 (m, 4H), 7.23-7.15 (m, 2H), 7.13 (s, 1H), 4.50-4.40 (m, 3H), 4.34-4.28 (m, 3H), 4.05 (s, 1H), 3.98 (s, 1H), 3.60-3.55 (m, 3H), 3.44-3.35 (m, 6H), 2.81 (s, 2H), 2.22 (s, 4H), 2.00-1.80 (m, 8H), 1.50-1.30 (m, 15H), 1.24 (s, 1H), 1.06 (s, 2H), 0.58 (d, J = 7.7 Hz, 2H), 0.47 (d, J = 5.1 Hz, 2H). |
| 41 | I-203 | 2-Amino-5-(1-[2-(2- | 997.70 | (400 MHz, CD₃OD) δ 8.25 (d, J = |
| | | (2-[3-[1-(2,6-dioxo | | 2.2 Hz, 1H), 8.00 (dd, J = 2.3, 1.2 |
| | | piperidin-3-yl)-3- | | Hz, 1H), 7.95 (s, 1H), 7.78 (d, J = |
| | | methyl-2-oxo-1,3- | | 1.0 Hz, 1H), 7.51 (t, J = 7.9 Hz, |
| | | benzodiazol-5-yl] | | 4H), 7.39 (dd, J = 13.2, 8.0 Hz, |
| | | propoxy]ethoxy)ethoxy] | | 4H), 6.98-6.91 (m, 2H), 6.87 (d, J = |
| | | ethyl]pyrazol-4-yl)- | | 7.8 Hz, 1H), 5.28 (dd, J = 12.7, |
| | | N-[(1S,2S)-2-([4-[(4- | | 5.4 Hz, 1H), 4.72-4.61 (m, 2H), |
| | | methylpiperazin-1- | | 4.43 (q, J = 7.2 Hz, 1H), 4.29 (t, J = |
| | | yl)methyl]-[1,1- | | 5.0 Hz, 2H), 3.98 (s, 1H), 3.85 (t, |
| | | biphenyl]-4-yl] | | J = 5.1 Hz, 2H), 3.60 (s, 4H), 3.58- |
| | | yl]methoxy)cyclopentyl] | | 3.53 (m, 4H), 3.48 (dd, J = 5.7, 3.3 |
| | | pyridine-3-carboxamide | | Hz, 2H), 3.36 (s, 5H), 3.06-2.71 (m, |
| | | | | 4H), 2.66 (t, J = 7.5 Hz, 3H), 2.61- |
| | | | | 2.35 (m, 6H), 2.30 (s, 3H), 2.15 (td, |
| | | | | J = 12.8, 5.5 Hz, 2H), 2.03 (dt, J = |
| | | | | 12.6, 6.9 Hz, 1H), 1.87-1.72 (m, |
| | | | | 5H), 1.61 (dq, J = 14.8, 7.5 Hz, 1H) |
| 42 | I-182 | 2-Amino-5-[1-[2- | 953.50 | (400 MHz, CD₃OD) δ 8.22 (d, J = |
| | | (2-[3-[1-(2,6- | | 2.3 Hz, 1H), 8.00-7.94 (m, 2H), |
| | | dioxopiperidin-3- | | 7.81-7.76 (m, 1H), 7.55-7.48 (m, |
| | | yl)-3-methyl-2-oxo- | | 4H), 7.43-7.34 (m, 4H), 6.92-6.83 |
| | | 1,3-benzodiazol-5- | | (m, 2H), 6.75 (dt, J = 8.1, 1.9 Hz, |
| | | yl]propoxy]ethoxy) | | 1H), 5.26 (ddd, J = 12.4, 5.4, 3.0 |
| | | ethyl]pyrazol-4-yl]- | | Hz, 1H), 4.72-4.59 (m, 2H), 4.46- |
| | | N-[(1S,2S)-2-([4- | | 4.37 (m, 1H), 4.31 (t, J = 5.0 Hz, |
| | | [(4- | | 2H), 4.02-3.93 (m, 1H), 3.86 (t, J = |
| | | methylpiperazin-1- | | 5.0 Hz, 2H), 3.63-3.58 (m, 2H), |
| | | yl)methyl]-[1,1- | | 3.57 (s, 2H), 3.54 (dd, J = 5.7, 2.7 |
| | | biphenyl]-4- | | Hz, 2H), 3.38 (t, J = 6.3 Hz, 2H), |
| | | yl]methoxy)cyclo- | | 2.95-2.84 (m, 2H), 2.84-2.72 (m, |
| | | pentyl]pyridine-3- | | 3H), 2.54 (s, 10H), 2.30 (s, 3H), |
| | | carboxamide | | 2.22-1.95 (m, 4H), 1.79 (m, 5H), |
| | | | | 1.66-1.53 (m, 1H) |
| 43 | I-217 | 2-Amino-5-[1-(2- | 909.40 | (400 MHz, CD₃OD) δ 8.27 (d, J = |
| | | [3-[1-(2,6- | | 2.2 Hz, 1H), 8.01 (s, 1H), 7.98 (s, |
| | | dioxopiperidin-3- | | 1H), 7.83 (d, J = 0.9 Hz, 1H), 7.54- |
| | | yl)-3-methyl-2-oxo- | | 7.46 (m, 4H), 7.42-7.33 (m, 4H), |
| | | 1,3-benzodiazol-5- | | 6.89-6.83 (m, 2H), 6.80-6.73 (m, |
| | | yl]propoxy]ethyl) | | 1H), 5.25 (ddd, J = 12.9, 5.5, 2.1 |
| | | pyrazol-4-yl]-N- | | Hz, 1H), 4.70-4.58 (m, 2H), 4.41 |
| | | [(1S,2S)-2-([4-[(4- | | (q, J = 7.3 Hz, 1H), 4.30 (t, J = 4.7 |
| | | methylpiperazin-1- | | Hz, 2H), 3.97 (dt, J = 6.6, 3.2 Hz, |
| | | yl)methyl]-[1,1- | | 1H), 3.77 (t, J = 5.0 Hz, 2H), 3.56 |
| | | biphenyl]-4- | | (s, 2H), 3.39 (t, J = 6.1 Hz, 2H), |
| | | yl]methoxy)cyclo- | | 3.31 (s, 3H), 2.99-2.66 (m, 4H), |
| | | pentyl]pyridine-3- | | 2.61 (t, J = 7.5 Hz, 2H), 2.60-2.40 |
| | | carboxamide | | (m, 6H), 2.29 (s, 3H), 2.22-1.95 (m, |
| | | | | 4H), 1.89-1.68 (m, 5H), 1.65-1.52 |
| | | | | (m, 1H) |
| 44 | I-204 | 2-Amino-5-(1-[2- | 997.50 | (400 MHz, CD₃OD) δ 8.24 (d, J = |
| | | [2-(2-[3-[1-(2,6- | | 2.2 Hz, 1H), 8.02-7.94 (m, 2H), |
| | | dioxopiperidin-3- | | 7.77 (t, J = 0.9 Hz, 1H), 7.55-7.47 |
| | | yl)-3-methyl-2-oxo- | | (m, 4H), 7.45-7.33 (m, 4H), 7.02- |
| | | 1,3-benzodiazol-4- | | 6.84 (m, 3H), 5.29 (dd, J = 12.3, 5.5 |
| | | yl]propoxy]ethoxy) | | Hz, 1H), 4.73-4.60 (m, 2H), 4.43 |
| | | ethoxy]ethyl] | | (q, J = 7.2 Hz, 1H), 4.29 (t, J = 5.1 |
| | | pyrazol-4-yl)-N-[(1S,2S)- | | Hz, 2H), 3.99 (t, J = 5.3 Hz, 1H), |
| | | 2-([4-[(4- | | 3.85 (t, J = 5.1 Hz, 2H), 3.60 (s, |
| | | methylpiperazin-1- | | 3H), 3.59-3.53 (m, 8H), 3.49 (t, J = |
| | | yl)methyl]-[1,1- | | 4.6 Hz, 2H), 3.41 (t, J = 6.0 Hz, |
| | | biphenyl]-4- | | 2H), 2.99-2.72 (m, 6H), 2.70-2.40 |
| | | yl]methoxy)cyclo- | | (m, 7H), 2.30 (s, 3H), 2.26-2.00 (m, |
| | | pentyl]pyridine-3- | | 3H), 1.89-1.72 (m, 5H), 1.62 (dd, J = |
| | | carboxamide | | 13.2, 7.1 Hz, 1H) |
| 45 | I-184 | 2-Amino-5-[1-[2- | 953.55 | (400 MHz, CD₃OD) δ 8.22 (s, 1H), |
| | | (2-[3-[1-(2,6- | | 7.95 (dd, J = 4.3, 2.3 Hz, 2H), 7.77 |
| | | dioxopiperidin-3- | | (d, J = 2.2 Hz, 1H), 7.56-7.47 (m, |
| | | yl)-3-methyl-2-oxo- | | 4H), 7.41 (d, J = 7.8 Hz, 2H), 7.37 |
| | | 1,3-benzodiazol-4- | | (d, J = 7.9 Hz, 2H), 6.94 (t, J = 7.7 |
| | | yl]propoxy]ethoxy) | | Hz, 1H), 6.88 (d, J = 7.8 Hz, 1H), |
| | | ethyl]pyrazol-4-yl]- | | 6.77 (d, J = 7.7 Hz, 1H), 5.26 (dt, J = |
| | | N-[(1S,2S)-2-([4- | | 11.4, 5.6 Hz, 1H), 4.73-4.61 (m, |
| | | [(4- | | 2H), 4.46-4.38 (m, 1H), 4.31 (t, J = |
| | | methylpiperazin-1- | | 5.1 Hz, 2H), 3.98 (s, 1H), 3.89-3.81 |
| | | yl)methyl]-[1,1- | | (m, 2H), 3.63-3.51 (m, 7H), 3.50 (s, |
| | | biphenyl]-4- | | 3H), 3.42 (dt, J = 6.5, 3.2 Hz, 2H), |
| | | yl]methoxy)cyclo- | | 2.91-2.83 (m, 3H), 2.82-2.72 (m, |
| | | pentyl]pyridine-3- | | 2H), 2.70-2.40 (m, 7H), 2.30 (s, |
| | | carboxamide | | 3H), 2.25-2.10 (m, 2H), 2.04 (dt, J = |
| | | | | 13.4, 6.9 Hz, 1H), 1.88-1.71 (m, |
| | | | | 5H), 1.61 (dq, J = 14.9, 7.7 Hz, |
| | | | | 1H). |
| 46 | I-218 | 2-Amino-5-[1-(2- | 909.45 | (400 MHz, CD₃OD) δ 8.27 (dd, J = |
| | | [3-[1-(2,6- | | 2.3, 1.2 Hz, 1H), 8.01 (d, J = 2.3 |
| | | dioxopiperidin-3- | | Hz, 1H), 7.93 (s, 1H), 7.82 (d, J = |
| | | yl)-3-methyl-2-oxo- | | 0.9 Hz, 1H), 7.49 (tt, J = 8.1, 2.4 |
| | | 1,3-benzodiazol-4- | | Hz, 4H), 7.43-7.31 (m, 4H), 6.98- |
| | | yl]propoxy]ethyl) | | 6.91 (m, 1H), 6.90 (d, J = 7.8 Hz, |
| | | pyrazol-4-yl]-N- | | 1H), 6.77 (dd, J = 7.3, 1.5 Hz, 1H), |
| | | [(1S,2S)-2-([4-[(4- | | 5.27 (dd, J = 12.6, 5.4 Hz, 1H), |
| | | methylpiperazin-1- | | 4.71-4.59 (m, 2H), 4.42 (q, J = 7.1 |
| | | yl)methyl]-[1,1- | | Hz, 1H), 4.29 (t, J = 5.0 Hz, 2H), |
| | | biphenyl]-4- | | 4.02-3.94 (m, 1H), 3.80 (t, J = 5.1 |
| | | yl]methoxy)cyclo- | | Hz, 2H), 3.55 (s, 2H), 3.48 (s, 3H), |
| | | pentyl]pyridine-3- | | 3.45-3.41 (m, 2H), 2.95-2.66 (m, |
| | | carboxamide | | 6H), 2.68-2.34 (m, 6H), 2.29 (s, |
| | | | | 3H), 2.24-1.93 (m, 4H), 1.89-1.68 |
| | | | | (m, 5H), 1.60 (dq, J = 15.2, 7.6 Hz, |
| | | | | 1H) |
| 47 | I-183 | 2-Amino-5-(1-[3- | 865.50 | (400 MHz, CD₃OD) δ 8.29 (d, J = |
| | | [1-(2,6- | | 2.4 Hz, 1H), 7.98 (d, J = 2.2 Hz, |
| | | dioxopiperidin-3- | | 1H), 7.91 (d, J = 1.8 Hz, 1H), 7.82 |
| | | yl)-3-methyl-2-oxo- | | (s, 1H), 7.55-7.39 (m, 6H), 7.32 (d, |
| | | 1,3-benzodiazol-4- | | J = 8.1 Hz, 2H), 7.05 (t, J = 7.8 Hz, |
| | | yl]propyl]pyrazol- | | 1H), 7.02-6.92 (m, 2H), 5.31 (dd, J = |
| | | 4-yl)-N-[(1S,2S)-2- | | 12.4, 5.3 Hz, 1H), 4.75-4.61 (m, |
| | | ([4-[(4- | | 2H), 4.50-4.40 (m, 1H), 4.24 (t, J = |
| | | methylpiperazin-1- | | 6.6 Hz, 2H), 3.98 (q, J = 5.0 Hz, |
| | | yl)methyl]-[1,1- | | 1H), 3.50 (d, J = 1.7 Hz, 2H), 3.41 |
| | | biphenyl]-4- | | (s, 3H), 2.98-2.85 (m, 3H), 2.85- |
| | | yl]methoxy)cyclo- | | 2.76 (m, 2H), 2.70-2.40 (m, 7H), |
| | | pentyl]pyridine-3- | | 2.32 (s, 3H), 2.23-2.02 (m, 6H), |
| | | carboxamide | | 1.92-1.69 (m, 3H), 1.67-1.53 (m, |
| | | | | 1H). |
| 78 | I-161 | (2S,4R)-1-[(2S)- | 1029.80 | (400 MHz, CD₃OD) δ 8.88 (s, 1H), |
| | | 3,3-dimethyl-2-(6- | | 8.75 (s, 1H), 7.61 (d, J = 7.9 Hz, |
| | | [4-[(4-[7-[trans-4- | | 2H), 7.48 (d, J = 8.1 Hz, 2H), 7.48- |
| | | hydroxycyclohexyl]- | | 7.36 (m, 5H), 4.65 (s, 1H), 4.62- |
| | | 2-[(3,3,3- | | 4.49 (m, 4H), 4.36 (d, J = 15.5 Hz, |
| | | trifluoropropyl) | | 1H), 3.19 (d, J = 11.0 Hz, 1H), 3.81 |
| | | amino]pyrrolo[2,3- | | (dd, J = 11.0, 3.8 Hz, 1H), 3.77- |
| | | d]pyrimidin-5- | | 3.69 (m, 3H), 3.58 (s, 2H), 2.66- |
| | | yl]phenyl)methyl] | | 2.50 (m, 9H), 2.48 (s, 3H), 2.38 (t, |
| | | piperazin-1- | | J = 7.5 Hz, 2H), 2.37-2.18 (m, 3H), |
| | | yl]hexanamido) | | 2.08-2.05 (m, 8H), 1.64 (q, J = 7.6 |
| | | butanoyl]-4-hydroxy-N- | | Hz, 2H), 1.60-1.50 (m, 4H), 1.35 |
| | | [[4-(4-methyl-1,3- | | (dt, J = 16.0, 8.1 Hz, 2H), 1.05 (s, |
| | | thiazol-5- | | 9H). |
| | | yl)phenyl]methyl] | | |
| | | pyrrolidine-2- | | |
| | | carboxamide | | |
| 49 | I-162 | (2S,4R)-1-[(2S)- | 1043.35 | (400 MHz, CD₃OD) δ 8.88 (s, 1H), |
| | | 3,3-dimethyl-2-(7- | | 8.75 (s, 1H), 7.61 (d, J = 8.0 Hz, |
| | | [4-[(4-[7-[trans-4- | | 2H), 7.51-7.36 (m, 7H), 4.65 (s, |
| | | hydroxycyclohexyl] | | 1H), 4.61-4.49 (m, 4H), 4.36 (d, J = |
| | | 2-[(3,3,3- | | 15.5 Hz, 1H), 3.92 (d, J = 11.0 |
| | | trifluoropropyl) | | Hz, 1H), 3.82 (dd, J = 10.9, 3.9 Hz, |
| | | amino]pyrrolo[2,3- | | 1H), 3.77-3.69 (m, 3H), 3.58 (s, |
| | | d]pyrimidin-5- | | 2H), 2.68-2.51 (m, 8H), 2.48 (s, |
| | | yl]phenyl)methyl] | | 3H), 2.38 (m, J = 8.0 Hz, 2H), 2.35- |
| | | piperazin-1- | | 2.18 (m, 4H), 2.18-2.05 (m, 7H), |
| | | yl]hetanamido) | | 1.63 (d, J = 7.9 Hz, 2H), 1.54 (m, |
| | | butanoyl]-4-hydroxy- | | 4H), 1.39-1.29 (m, 5H), 1.05 (s, |
| | | N-[[4-(4-methyl- | | 9H) |
| | | 1,3-thiazol-5- | | |
| | | yl)phenyl]methyl] | | |
| | | pyrrolidine-2- | | |
| | | carboxamide | | |
| 50 | I-165 | (2S,4R)-1-[(2S)- | 1061.80 | (400 MHz, CD₃OD) δ 8.86 (s, 1H), |
| | | 3,3-dimethyl-2-[2- | | 8.75 (s, 1H), 7.60 (d, J = 8.0 Hz, |
| | | [2-(2-[4-[(4-[7- | | 2H), 7.47-7.35 (m, 7H), 4.70 (s, |
| | | [trans-4- | | 1H), 4.62-4.49 (m, 4H), 4.36 (d, J = |
| | | hydroxycyclohexyl] | | 15.5 Hz, 1H), 4.12-4.00 (m, 2H), |
| | | 2-[(3,3,3- | | 3.91-3.78 (m, 2H), 3.75-3.64 (m, |
| | | trifluoropropyl) | | 9H), 3.55 (s, 2H), 2.71-2.50 (m, |
| | | amino]pyrrolo[2,3- | | 12H), 2.48 (s, 3H), 2.27-2.03 (m, |
| | | d]pyrimidin-5- | | 8H), 1.61-1.49 (m, 2H), 1.03 (s, |
| | | yl]phenyl)methyl] | | 9H). |
| | | piperazin-1- | | |
| | | yl]ethoxy)ethoxy] | | |
| | | acetamido]butanoyl]- | | |
| | | 4-hydroxy-N-[[4- | | |
| | | (4-methyl-1,3- | | |
| | | thiazol-5- | | |
| | | yl)phenyl]methyl] | | |
| | | pyrrolidine-2- | | |
| | | carboxamide | | |
| 51 | I-166 | (2S,4R)-1-[(2S)- | 1105.90 | (400 MHz, CD₃OD) δ 8.88 (s, 1H), |
| | | 3,3-dimethyl-2-(2- | | 8.76 (s, 1H), 7.61 (d, J = 8.0 Hz, |
| | | [2-[2-(2-[4-[(4-[7- | | 2H), 7.48-7.37 (m, 7H), 4.71 (s, |
| | | [trans-4- | | 1H), 4.58 (dd, J = 8.8, 7.1 Hz, 2H), |
| | | hydroxycyclohexyl]- | | 4.52 (d, J = 6.5 Hz, 2H), 4.38 (s, |
| | | 2-[(3,3,3- | | 1H), 4.11-3.97 (m, 2H), 3.92-3.79 |
| | | trifluoropropyl) | | (m, 2H), 3.78-3.53 (m, 16H), 2.70- |
| | | amino]pyrrolo[2,3- | | 2.51 (m, 11H), 2.48 (s, 3H), 2.36- |
| | | d]pyrimidin-5- | | 2.28 (m, 1H), 2.20-2.04 (m, 7H), |
| | | yl]phenyl)methyl] | | 1.54 (p, J = 10.4 Hz, 2H), 1.05 (s, |
| | | piperazin-1- | | 9H). |
| | | yl]ethoxy)ethoxy] | | |
| | | ethoxy]acetamido) | | |
| | | butanoyl]-4-hydroxy- | | |
| | | N-[[4-(4-methyl- | | |
| | | 1,3-thiazol-5- | | |
| | | yl)phenyl]methyl] | | |
| | | pyrrolidine-2- | | |
| | | carboxamide | | |
| 52 | I-173 | (2S,4R)-1-[(2S)- | 1149.95 | (400 MHz, CD₃OD) δ 8.88 (s, 1H), |
| | | 3,3-dimethyl-2-(14- | | 8.76 (s, 1H), 7.61 (d, J = 7.9 Hz, |
| | | [4-[(4-[7-[(1r,4r)-4- | | 2H), 7.48-7.37 (m, 7H), 4.70 (s, |
| | | hydroxycyclohexyl]- | | 1H), 4.61-4.49 (m, 4H), 4.36 (d, J = |
| | | 2-[(3,3,3- | | 15.6 Hz, 1H), 4.04 (d, J = 4.1 Hz, |
| | | trifluoropropyl) | | 2H), 3.91-3.79 (m, 2H), 3.75-3.55 |
| | | amino]pyrrolo[2,3- | | (m, 20H), 2.66-2.51 (m, 10H), 2.48 |
| | | d]pyrimidin-5- | | (s, 3H), 2.32 (s, 1H), 2.27-2.19 (m, |
| | | yl]phenyl)methyl] | | 1H), 2.18-2.09 (m, 7H), 1.55 (q, J = |
| | | piperazin-1-yl]- | | 10.1, 9.5 Hz, 2H), 1.05 (s, 9H). |
| | | 3,6,9,12- | | |
| | | tetraoxatetradecanamido) | | |
| | | butanoyl]-4- | | |
| | | hydroxy-N-[[4-(4- | | |
| | | methyl-1,3-thiazol- | | |
| | | 5- | | |
| | | yl)phenyl]methyl] | | |
| | | pyrrolidine-2- | | |
| | | carboxamide | | |

### Step 2: (4-(((4-(1-(cyclopropylmethyl)-1H-indazol-5-yl)benzyl)amino)methyl)phenyl)(1′-(4-(7-(trans-4-hydroxycyclohexyl)-2-((3,3,3-trifluoropropyl)amino)-7H-pyrrolo[2,3-d]pyrimidin-5-yl)benzyl)-[4,4′-bipiperidin]-1-yl)methanone formic acid

To a stirred mixture of tert-butyl N-([4-[1-(cyclopropylmethyl)indazol-5-yl]phenyl]methyl)-N-[(4-[1-[(4-[7-[trans-4-hydroxycyclohexyl]-2-[(3,3,3-trifluoropropyl)amino]pyrrolo[2,3-d]pyrimidin-5-yl]phenyl)methyl]-[4,4-bipiperidine]-1-carbonyl]phenyl)methyl]carbamate (30.0 mg, 0.028 mmol) in CH₂Cl₂ (5.00 mL) was added 4 M HCl (gas) in 1,4-dioxane (5.00 mL) dropwise at 25° C. The resulting mixture was stirred for 1 h at 25° C. and concentrated under reduced pressure. The residue was purified by reversed phase flash chromatography with the following conditions: Column: Xselect CSH OBD Column 30×150 mm 5 um; Mobile Phase A: water (plus 0.1% FA), Mobile Phase B: ACN; Flow rate: 60 mL/min; Gradient: 35% B to 50% B in 7 min; detector: UV 220/254 nm; desired fractions were collected at 6.18 min and concentrated under reduced pressure to afford (4-(((4-(1-(cyclopropylmethyl)-1H-indazol-5-yl)benzyl)amino)methyl)phenyl)(1′-(4-(7-(trans-4-hydroxycyclohexyl)-2-((3,3,3-trifluoropropyl)amino)-7H-pyrrolo[2,3-d]pyrimidin-5-yl)benzyl)-[4,4′-bipiperidin]-1-yl)methanone formic acid (15.0 mg, 55%) as a colorless solid. ¹H NMR (400 MHz, CD₃OD) δ 8.78 (s, 1H), 8.52 (s, 1H), 8.09 (s, 1H), 8.02 (s, 1H), 7.78-7.64 (m, 6H), 7.60-7.50 (m, 7H), 7.46 (d, J=7.8 Hz, 2H), 4.77-4.50 (m, 2H), 4.35 (d, J=6.9 Hz, 2H), 4.21-4.14 (m, 6H), 3.78-3.68 (m, 4H), 3.43 (d, J=11.9 Hz, 2H), 3.09 (s, 1H), 2.85-2.72 (m, 2H), 2.59 (dt, J=11.3, 7.3Hz, 2H), 2.20-2.05 (m, 6H), 2.04-1.85 (m, 3H), 1.70 (s, 1H), 1.61-1.16 (m, 10H), 0.58 (dt, J=8.0, 3.1 Hz, 2H), 0.46 (q, J=5.7, 5.3 Hz, 2H). LC/MS (ESI, m/z): [(M+1)]⁺=978.75.

The following examples in Table 48 were synthesized according to the above procedure:

TABLE 48

| | | | MS: | |
|---|---|---|---|---|
| Examples | I-# | Name | [(M + 1)]⁺ | ¹H NMR |
| 53 | I-181 | trans-4-[5-[4-([4-[1-(4- | 950.75 | (400 MHz, CD₃OD) δ 8.76 (s, 1H), 8.07 |
| MER-283 | | [[([4-[1-(Cyclopro- | | (d, J = 0.9 Hz, 1H), 8.01 (s, 1H), 7.72 (dd, |
| | | pylmethyl)indazol-5- | | J = 8.8, 1.7 Hz, 1H), 7.69-7.58 (m, 7H), |
| | | yl]phenyl]methyl)ami- | | 7.51-7.42 (m, 7H), 4.62-4.49 (m, 1H), |
| | | no]methyl]benzoyl)azetidin- | | 4.37 (d, J = 17.2 Hz, 1H), 4.33 (s, 2H), |
| | | 3-yl]piperidin-1- | | 4.21 (t, J = 9.4 Hz, 1H), 4.08 (dd, J = 9.2, |
| | | yl]methyl)phenyl]-2- | | 5.8 Hz, 1H), 3.94-3.82 (m, 5H), 3.78- |
| | | [(3,3,3-trifluoropropyl)amino] | | 3.66 (m, 3H), 3.60 (s, 2H), 3.00 (t, J = |
| | | pyrrolo[2,3-d]pyrimidin-7- | | 12.2 Hz, 2H), 2.59 (qt, J = 11.2, 7.3 Hz, |
| | | yl]cyclohexan-1-ol | | 2H), 2.47 (d, J = 7.5 Hz, 1H), 2.23-2.03 |
| | | | | (m, 8H), 1.80-1.64 (m, 2H), 1.60-1.45 |
| | | | | (m, 3H), 1.42-1.18 (m, 3H), 0.63-0.52 |
| | | | | (m, 2H), 0.45 (dt, J = 6.3, 4.5 Hz, 2H). |
| 54 | I-180 | trans-4-[5-(4-[[1-(4- | 992.65 | (400 MHz, CD₃OD) δ 8.92 (s, 1H), 8.43- |
| MER-281 | | [[([4-[2-(Cyclopro- | | 8.35 (m, 1H), 8.01 (s, 1H), 7.83 (d, J = |
| | | pylmethyl)indazol-5-yl]-3- | | 7.9 Hz, 2H), 7.75-7.60 (m, 6H), 7.58- |
| | | methylphenyl]methyl)ami- | | 7.49 (m, 2H), 7.45 (s, 1H), 7.39 (s, 2H), |
| | | no]methyl]benzoyl)-[4,4- | | 7.29 (d, J = 8.8 Hz, 1H), 4.80-4.60 (m, |
| | | bipiperidin]-1-yl]methyl]phenyl)- | | 2H), 4.43-4.29 (m, 8H), 3.88 (t, J = 6.9 |
| | | 2-[(3,3,3-trifluoropropyl)amino] | | Hz, 2H), 3.79-3.64 (m, 4H), 3.64-3.53 |
| | | pyrrolo[2,3-d]pyrimidin-7- | | (m, 3H), 3.13-2.99 (m, 2H), 2.91-2.78 |
| | | yl]cyclohexan-1-ol | | (m, 1H), 2.75-2.65 (m, 2H), 2.35 (s, 3H), |
| | | hydrochloride | | 2.22-2.00 (m, 7H), 1.92 (s, 1H), 1.73 (s, |
| | | | | 1H), 1.66-1.45 (m, 6H), 1.38-1.20 (m, |
| | | | | 2H), 0.72 (t, J = 7.1 Hz, 2H), 0.54 (d, J = |
| | | | | 5.2 Hz, 2H). |
| 55 | I-191 | trans-4-[5-[4-([4-[1-(4- | 964.60 | (400 MHz, CD₃OD) δ 8.78 (s, 1H), 8.32 |
| MER-282 | | [[([4-[2-(cyclopro- | | (s, 1H), 7.70-7.57 (m, 6H), 7.48 (d, J = |
| | | pylmethyl)indazol-5-yl]-3- | | 8.0 Hz, 2H), 7.44 (s, 1H), 7.40 (d, J = 7.9 |
| | | methylphenyl]methyl)ami- | | Hz, 2H), 7.29-7.25 (m, 2H), 7.23 (d, J = |
| | | no]methyl]benzoyl)azetidin- | | 1.8 Hz, 2H), 4.62-4.50 (m, 1H), 4.42 (t, |
| | | 3-yl]piperidin-l-yl]methyl)phenyl]- | | J = 8.8 Hz, 1H), 4.34 (d, J = 7.2 Hz, 2H), |
| | | 2-[(3,3,3-trifluoropropyl)amino] | | 4.23 (t, J = 9.4 Hz, 1H), 4.14-4.04 (m, |
| | | pyrrolo[2,3-d]pyrimidin-7- | | 1H), 3.95-3.89 (m, 1H), 3.88 (s, 2H), |
| | | yl]cyclohexan-1-ol | | 3.81 (s, 2H), 3.77-3.65 (m, 4H), 3.60 (s, |
| | | | | 2H), 3.00 (t, J = 12.0 Hz, 2H), 2.68-2.41 |
| | | | | (m, 3H), 2.29 (s, 3H), 2.21-2.02 (m, 8H), |
| | | | | 1.81-1.63 (m, 2H), 1.61-1.43 (m, 3H), |
| | | | | 1.32-1.20 (m, 2H), 0.75-0.66 (m, 2H), |
| | | | | 0.53 (dt, J = 6.2, 4.6 Hz, 2H). |

### EXAMPLE 56: 3-(3-methyl-2-oxo-5-[2-[4-([4-[(4-[7-[trans-4-hydroxycyclohexyl]-2-[(3,3,3-trifluoropropyl)amino]-7H-pyrrolo[2,3-d]pyrimidin-5-yl]phenyl)methyl]piperazin-1-yl]methyl)-1H-1,2,3-triazol-1-yl]ethyl]-2,3-dihydro-1H-1,3-benzodiazol-1-yl)piperidine-2,6-dione (I-114)

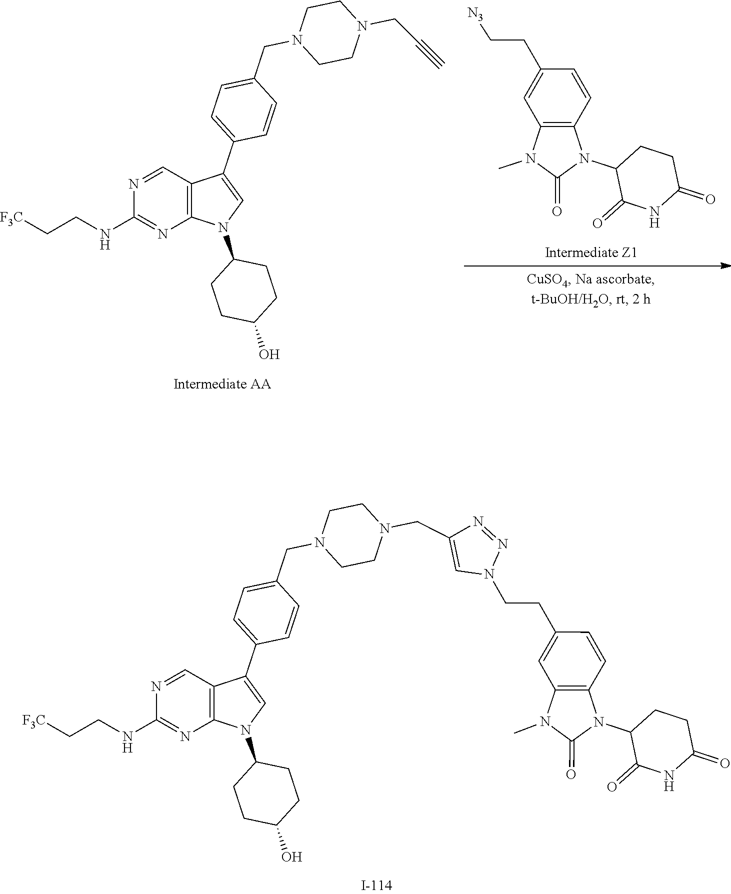 [SMILES: C#CCN1CCN(Cc2ccc(-c3cn([C@H]4CC[C@H](O)CC4)c4nc(NCCC(F)(F)F)ncc34)cc2)CC1 ; Cn1c(=O)n(C2CCC(=O)NC2=O)c2ccc(CCN=[N+]=[N-])cc21 ; Cn1c(=O)n(C2CCC(=O)NC2=O)c2ccc(CCn3cc(CN4CCN(Cc5ccc(-c6cn([C@H]7CC[C@H](O)CC7)c7nc(NCCC(F)(F)F)ncc67)cc5)CC4)nn3)cc21]

To a stirred mixture of trans-4-[5-(4-[[4-(prop-2-yn-1-yl)piperazin-1-yl]methyl]phenyl)-2-[(3,3,3-trifluoropropyl)amino]-7H-pyrrolo[2,3-d]pyrimidin-7-yl]cyclohexan-1-ol (50.0 mg, 0.092 mmol) and 3-[5-(2-azidoethyl)-3-methyl-2-oxo-2,3-dihydro-1H-1,3-benzodiazol-1-yl]piperidine-2,6-dione (30.4 mg, 0.092 mmol) in t-BuOH (8.00 mL), DCM (1.00 mL) and H₂O (4.00 mL) were added CuSO₄.5H₂O (9.24 mg, 0.037 mmol) and sodium ascorbate (14.7 mg, 0.074 mmol) at room temperature under nitrogen atmosphere. After stirring for additional 2 h, the resulting mixture was concentrated under reduced pressure. The residue was purified by Prep-HPLC with the following conditions (Column: XBridge Shield RP18 OBD Column 30×150 mm, 5 um; Mobile Phase A: water (plus 10 mmol/L NH₄HCO₃), Mobile Phase B: ACN; Flow rate: 60 mL/min; Gradient: 7% B to 25% B in 7 min; detector: 254/220 nm; desired fractions were collected at 6.5 min and lyophilized to afford 3-(3-methyl-2-oxo-5-[2-[4-([4-[(4-[7-[trans-4-hydroxycyclohexyl]-2-[(3,3,3-trifluoropropyl)amino]-7H-pyrrolo [2,3-d]pyrimidin-5-yl]phenyl)methyl]piperazin-1-yl]methyl)-1H-1,2,3-triazol-1-yl]ethyl]-2,3-dihydro-1H-1,3-benzodiazol-1-yl)piperidine-2,6-dione (23.1 mg, 29%) as a white solid. ¹H NMR (400 MHz, DMSO-d₆) δ 11.09 (s, 1H), 8.88 (s, 1H), 7.86 (s, 1H), 7.67-7.60 (m, 3H), 7.30 (d, J=8.0 Hz, 2H), 7.06 (t, J=5.9 Hz, 1H), 7.03-6.97 (m, 2H), 6.84 (dd, J=8.1, 1.6 Hz, 1H), 5.33 (dd, J=12.7, 5.3 Hz, 1H), 4.71 (d, J=4.5 Hz, 1H), 4.59 (t, J=7.2 Hz, 2H), 4.48-4.37 (m, 1H), 3.57 (m, 3H), 3.51 (s, 2H), 3.44 (s, 2H), 3.30 (s, 3H), 3.18 (t, J=7.3 Hz, 2H), 2.94-2.81 (m, 1H), 2.76-2.56 (m, 4H), 2.46-2.28 (m, 7H), 2.03-1.85 (m, 8H), 1.43-1.32 (m, 2H). LC/MS (ESI, m/z): [(M+1)]⁺=869.30.

The following example in Table 49 were synthesized according to the above procedure:

TABLE 49

| | | | MS: | |
|---|---|---|---|---|
| Example | I-# | Name | [(M + 1)]⁺ | ¹H NMR |
| 57 | I-113 | 3-(3-Methyl-2-oxo-5-[[4- | 855.50 | (400 MHz, DMSO-d₆) δ 11.12 (s, |
| | | ([4-[(4-[7-[trans-4- | | 1H), 8.87 (s, 1H), 8.02 (s, 1H), 7.65- |
| | | hydroxycyclohexyl]-2- | | 7.59 (m, 3H), 7.29 (d, J = 7.9 Hz, |
| | | [(3,3,3-trifluoropropyl)amino]-7H- | | 2H), 7.22 (s, 1H), 7.12 (d, J = 8.1 |
| | | pyrrolo[2,3-d]pyrimidin-5- | | Hz, 1H), 7.09-7.03 (m, 2H), 5.56 (s, |
| | | yl]phenyl)methyl]piperazin- | | 2H), 5.37 (dd, J = 12.8, 5.4 Hz, 1H), |
| | | 1-yl]methyl)-1H-1,2,3- | | 4.71 (d, J = 4.4 Hz, 1H), 4.42 (s, |
| | | triazol-1-yl]methyl]-2,3- | | 1H), 3.62-3.50 (m, 5H), 3.44 (s, |
| | | dihydro-1H-1,3-benzodiazol-1- | | 2H), 2.92-2.83 (m, 1H), 2.80-2.52 |
| | | yl)piperidine-2,6-dione | | (m, 4H), 2.50-2.30 (m, 9H), 2.04- |
| | | | | 1.88 (m, 9H), 1.36 (q, J = 12.1 Hz, |
| | | | | 2H) |

### EXAMPLE 58: 1-(5-[3-[5-(dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylic acid (I-167)

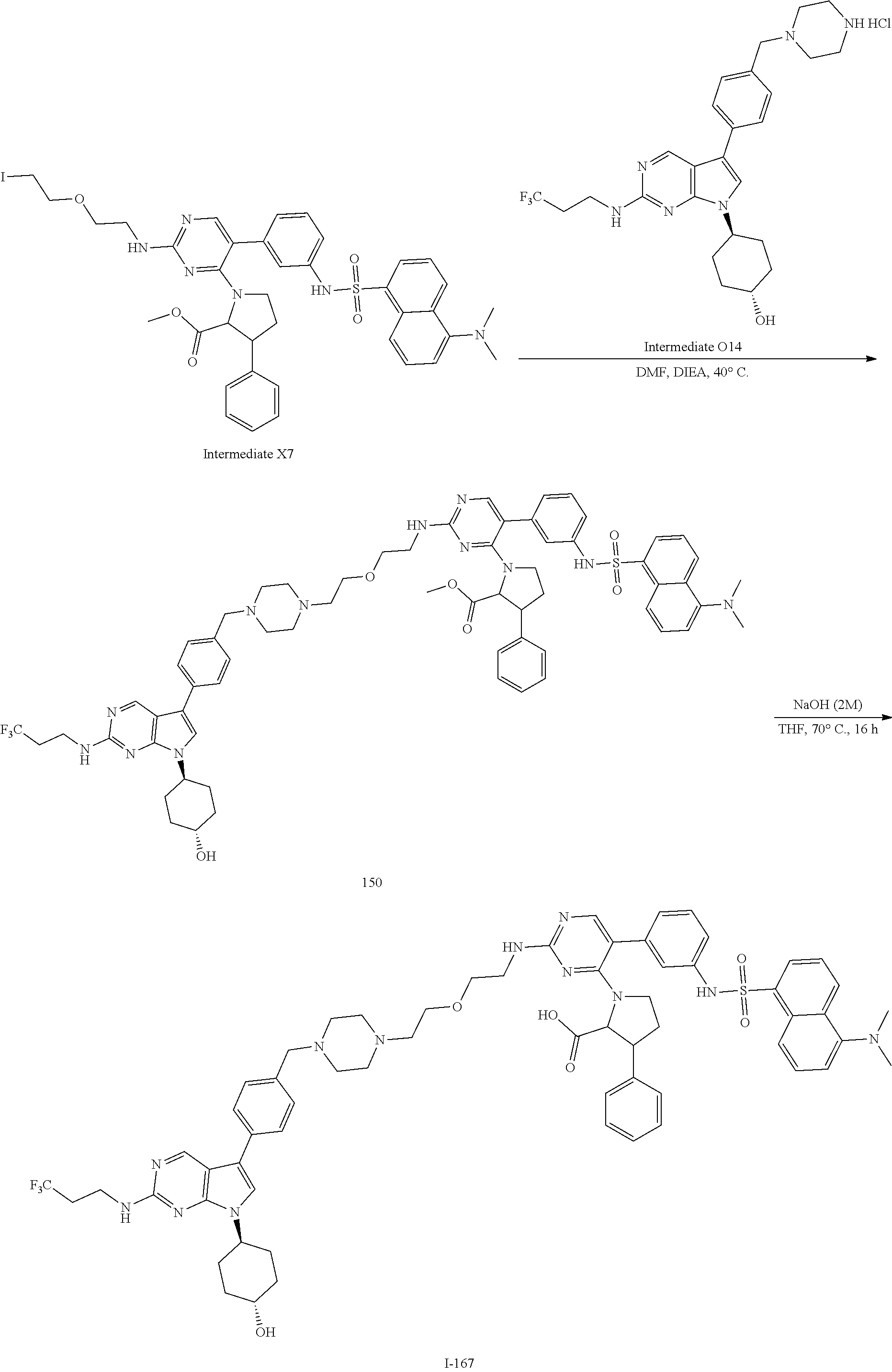

### Step 1: Methyl 1-(5-[3-[5-(dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylate

To a stirred solution of methyl 1-(5-[3-[5-(dimethylamino)naphthalene-1-sulfonamido]phenyl]-2-[[2-(2-iodoethoxy)ethyl]amino]pyrimidin-4-yl)-3-phenylpyrrolidine-2-carboxylate (200 mg, 0.25 mmol) and trans-4-[5-[4-(piperazin-1-ylmethyl)phenyl]-2-[(3,3,3-trifluoropropyl)amino]pyrrolo[2,3-d]pyrimidin-7-yl]cyclohexan-1-ol hydrochloride (123 mg, 0.25 mmol) in DMF (4.00 mL) was added DIEA (158 mg, 1.22 mmol) at room temperature under nitrogen atmosphere. The resulting solution was stirred for 16 h at 40° C. Upon completion, the solution was purified by reversed phase flash chromatography with the following conditions: Column: WelFlash™ C18-I, 20-40 um, 120 g; Eluent A: water (plus 10 mmol/L NH₄HCO₃); Eluent B: ACN; Gradient: 50%-75% B in 20 min; Flow rate: 55 mL/min; Detector: UV 220/254 nm; desired fractions were collected at 70% B and concentrated under reduced pressure to afford methyl-1-(5-[3-[5-(dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylate (200 mg, 69%) as a light yellow solid. ¹H NMR (400 MHz, CD₃OD) δ 8.72 (d, J=5.7 Hz, 1H), 8.47 (dd, J=8.6, 3.1 Hz, 2H), 8.24 (d, J=7.2 Hz, 1H), 7.62 (t, J=8.1 Hz, 1H), 7.56-7.13 (m, 16H), 6.93-6.75 (m, 2H), 4.65-4.49 (m, 2H), 3.80-3.70 (m, 4H), 3.65-3.38 (m, 10H), 3.18 (s, 2H), 2.75 (m, 6H), 2.70-2.40 (m, 13H), 2.18-2.00 (m, 7H), 1.55 (q, J=9.6 Hz, 2H). LC/MS (ESI, m/z): [(M+1)]⁺=1195.60.

The following intermediates in Table 50 were synthesized according to the above procedure:

TABLE 50

| Inter- | | | MS: | |
|---|---|---|---|---|
| mediate | Structure | Name | [(M + 1)]⁺ | ¹H NMR |
| 150-1 | 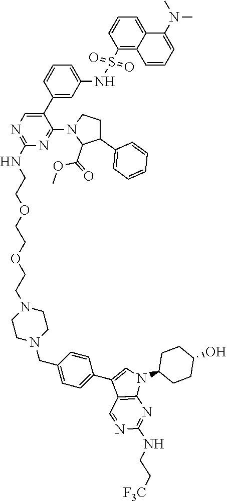 [SMILES: COC(=O)C1C(c2ccccc2)CCN1c1nc(NCCOCCOCCN2CCN(Cc3ccc(-c4cn([C@H]5CC[C@H](O)CC5)c5nc(NCCC(F)(F)F)ncc45)cc3)CC2)ncc1-c1cccc(NS(=O)(=O)c2cccc3c(N(C)C)cccc23)c1] | Methyl 1-(5-[3-[5- (dimethylamino) naphthalene-1- sulfonamido]phenyl]-2- ([2-[2-(2-[4-[(4-[7- [trans-4- hydroxycyclohexyl]-2- [(3,3,3- trifluoropropyl)amino] pyrrolo[2,3-d]pyrimidin- 5- yl]phenyl)methyl] piperazin-1- yl]ethoxy)ethoxy]ethyl] amino)pyrimidin-4-yl)- 3-phenylpyrrolidine-2- carboxylate | 1239.80 | (400 MHz, CD₃OD) δ 8.73 (s, 1H), 8.46 (dd, J = 8.8, 6.0 Hz, 2H), 8.22 (dd, J = 7.4, 1.2 Hz, 1H), 7.64-7.53 (m, 3H), 7.52-7.40 (m, 2H), 7.40-7.04 (m, 12H), 6.98-6.74 (m, 2H), 4.69-4.48 (m, 2H), 3.78-3.68 (m, 4H), 3.66-3.52 (m, 11H), 3.46-3.34 (m, 2H), 3.17 (s, 3H), 2.79-2.44 (m, 18H), 2.12 (m, 3H), 2.02 (m, 4H), 1.93-1.83 (m, 1H), 1.53 (m, 2H). |
| 150-2 | 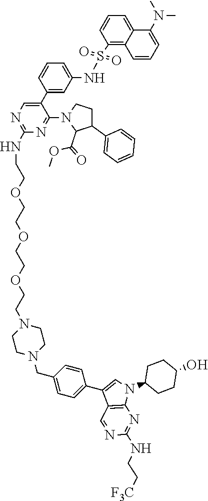 | Methyl 1-(5-[3-[5- (dimethylamino) naphthalene-1- sulfonamido]phenyl]-2- (12-[4-[(4-[7-[trans-4- hydroxycyclohexyl]-2- [(3,3,3- trifluoropropyl)amino] pyrrolo[2,3-d]pyrimidin- 5- yl]phenyl)methyl] piperazin-1-yl]-4,7,10- trioxa-1-azadodecan-1- yl)pyrimidin-4-yl)-3- phenylpyrrolidine-2- carboxylate | 1283.80 | (400 MHz, CD₃OD) δ 8.73 (d, J = 3.7 Hz, 1H), 8.47 (dd, J = 8.8, 4.8 Hz, 2H), 8.25-8.20 (m, 1H), 7.70-7.60 (m, 3H), 7.52-7.42 (m, 2H), 7.41-7.08 (m, 11H), 6.87 (s, 2H), 4.65 (d, J = 8.2 Hz, 1H), 4.57-4.49 (m, 1H), 3.80-3.70 (m, 5H), 3.64-3.48 (m, 15H), 3.46-3.35 (m, 2H), 3.18 (s, 3H), 2.80- 2.65 (m, 8H), 2.60-2.45 (m, 10H), 2.17-1.98 (m, 7H), 1.91-1.87 (m, 1H), 1.60-1.50 (m, 2H). |

### Step 2: 1-(5-[3-[5-(Dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylic acid

To a stirred solution of methyl-1-(5-[3-[5-(dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylate (50.0 mg, 0.042 mmol) in THF (5.00 mL) was added NaOH (2 M, 0.20 mL) at room temperature under nitrogen atmosphere. The resulting solution was stirred for 48 h at 70° C. Upon completion, the mixture was cooled down to room temperature. The mixture was acidified to pH 6 with HCl (4 M aq.). The resulting solution was purified by Prep-HPLC with the following conditions: Column: XB ridge Shield RP18 OBD Column, 30×150 mm, 5 um; Mobile Phase A: water (plus 10 mmol/L NH₄HCO₃), Mobile Phase B:ACN; Flow rate:60 mL/min; Gradient: 30% B to 55% B in 7 min; detector: 254/220 nm; desired fractions were collected at 6.5 min and concentrated under reduced pressure to afford 1-(5-[3-[5-(dimethylamino)naphthalene-1-sulfonamido]phenyl]-2-[[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethyl]amino]pyrimidin-4-yl)-3-phenylpyrrolidine-2-carboxylic acid (25.0 mg, 51%) as a white solid. ¹H NMR (400 MHz, DMSO-d₆) δ 10.68 (s, 1H), 8.88 (s, 1H), 8.41 (d, J=8.5 Hz, 2H), 8.20 (d, J=7.3 Hz, 1H), 7.71-7.51 (m, 5H), 7.42 (s, 1H), 7.36-7.23 (m, 7H), 7.18 (s, 2H), 7.05 (d, J=5.7 Hz, 2H), 6.87 (s, 2H), 6.53 (s, 1H), 4.65 (d, J=8.1 Hz, 2H), 4.41 (d, J=11.8 Hz, 1H), 3.62-3.49 (m, 5H), 3.49-3.00 (m, 9H), 2.75-2.56 (m, 9H), 2.50-2.03 (m, 11H), 2.03-1.85 (m, 6H), 1.37 (q, J=13.0, 12.2 Hz, 2H). LC/MS (ESI, m/z): [(M+1)]⁺=1181.60.

The following example in Table 51 were synthesized according to the above procedure:

TABLE 51

| | | | MS: | |
|---|---|---|---|---|
| Example | I-# | Name | [(M + 1)]⁺ | ¹H NMR |
| 59 | I-168 | 1-(5-[3-[5- | 1225.60 | (400 MHz, DMSO-d₆) δ 8.87 (s, |
| | | (Dimethylamino)naphthalene- | | 1H), 8.50-8.33 (m, 2H), 8.19 (d, J = |
| | | 1-sulfonamido]phenyl]-2-([2- | | 7.3 Hz, 1H), 7.65-7.62 (m, 4H), |
| | | [2-(2-[4-[(4-[7-[trans-4- | | 7.60-7.54 (m, 1H), 7.44 (s, 1H), |
| | | hydroxycyclohexyl]-2-[(3,3,3 - | | 7.37-7.14 (m, 10H), 7.05 (t, J = 5.9 |
| | | trifluoropropyl)amino]pyrrolo | | Hz, 2H), 6.87 (d, J = 7.8 Hz, 3H), |
| | | [2,3-d]pyrimidin-5- | | 4.69 (s, 1H), 4.61 (d, J = 7.8 Hz, |
| | | yl]phenyl)methyl]piperazin-1- | | 1H), 4.44 (d, J = 11.6 Hz, 1H), 3.63- |
| | | yl]ethoxy)ethoxy]ethyl]ami- | | 3.55 (m, 4H), 3.43 (s, 3H), 2.76 (s, |
| | | no)pyrimidin-4-yl)-3-phenyl | | 2H), 2.71 (s, 3H), 2.68-2.54 (m, |
| | | pyrrolidine-2-carboxylic acid | | 4H), 2.45 (s, 9H), 2.19 (s, 1H), 2.00- |
| | | | | 1.88 (m, 9H), 1.77 (m, 1H), 1.37 (s, |
| | | | | 9H), 1.25 (s, 1H). |
| 60 | I-169 | 1-(5-[3-[5- | 1269.60 | (400 MHz, DMSO-d₆) δ 10.70 (br, |
| | | (dimethylamino)naphthalene- | | 1H), 8.87 (s, 1H), 8.45 (s, 1H), 8.38 |
| | | 1-sulfonamido]phenyl]-2-(12- | | (d, J = 8.2 Hz, 1H), 8.18 (d, J = 7.3 |
| | | [4-[(4-[7-[trans-4-hydroxy- | | Hz, 1H), 7.69-7.51 (m, 6H), 7.45 (d, |
| | | cyclohexyl]-2-[(3,3,3- | | J = 8.1 Hz, 1H), 7.35-7.20 (m, 10H), |
| | | trifluoropropyl)amino]pyrrolo | | 7.04 (d, J = 6.4 Hz, 2H), 6.95-6.76 |
| | | [2,3-d]pyrimidin-5- | | (m, 2H), 4.70 (s, 1H), 4.60 (s, 1H), |
| | | yl]phenyl)methyl]piperazin-1- | | 4.39 (m, 2H), 3.60-3.55 (m, 5H), |
| | | yl]-4,7,10-trioxa-1- | | 3.53-3.45 (m, 15H), 2.76 (s, 3H), |
| | | azadodecan-1-yl)pyrimidin-4- | | 2.74-2.68 (m, 4H), 2.50-2.30 (m, |
| | | yl)-3-phenylpyrrolidine-2- | | 9H), 2.00-1.94 (m, 4H), 1.93-1.89 |
| | | carboxylic acid | | (m, 4H), 1.40-1.30 (m, 3H), 1.28- |
| | | | | 1.22 (m, 2H). |

### EXAMPLE 61: (2S,4R)-1-[(2S)-2-[(1-fluorocyclopropyl)formamido]-3,3-dimethylbutanoyl]-4-hydroxy-N-[[4-(4-methyl-1,3-thiazol-5-yl)-2-(4-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]butoxy)phenyl]methyl]pyrrolidine-2-carboxamide (I-179)

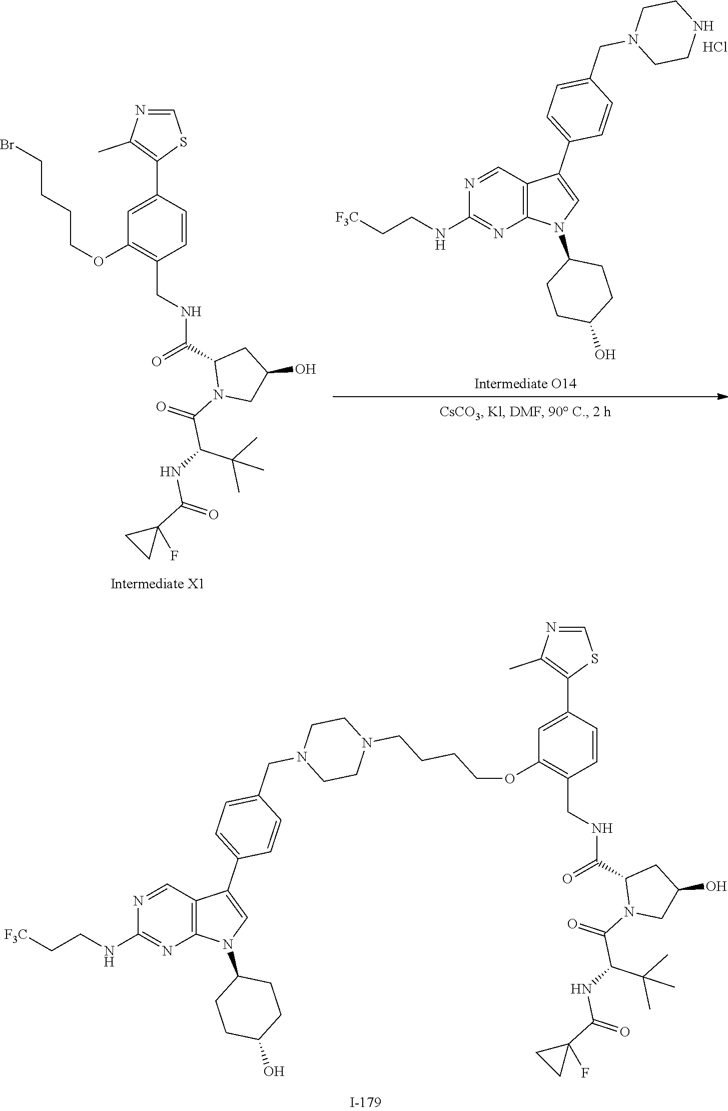 [SMILES: Cc1ncsc1-c1ccc(CNC(=O)[C@@H]2C[C@@H](O)CN2C(=O)[C@@H](NC(=O)C2(F)CC2)C(C)(C)C)c(OCCCCBr)c1 ; O[C@H]1CC[C@H](n2cc(-c3ccc(CN4CCNCC4)cc3)c3cnc(NCCC(F)(F)F)nc32)CC1 ; Cl ; Cc1ncsc1-c1ccc(CNC(=O)[C@@H]2C[C@@H](O)CN2C(=O)[C@@H](NC(=O)C2(F)CC2)C(C)(C)C)c(OCCCCN2CCN(Cc3ccc(-c4cn([C@H]5CC[C@H](O)CC5)c5nc(NCCC(F)(F)F)ncc45)cc3)CC2)c1]

To a stirred solution of (2S,4R)—N-[[2-(4-bromobutoxy)-4-(4-methyl-1,3-thiazol-5-yl)phenyl]methyl]-1-[(2S)-2-[(1-fluorocyclopropyl)formamido]-3,3-dimethylbutanoyl]-4-hydroxypyrrolidine-2-carboxamide (59.0 mg, 0.088 mmol) and trans-4-[5-[4-(piperazin-1-ylmethyl)phenyl]-2-[(3,3,3-trifluoropropyl)amino]pyrrolo[2,3-d]pyrimidin-7-yl]cyclohexan-1-ol hydrochloride (57.2 mg, 0.11 mmol) in DMF (2.00 mL) were added Cs₂CO₃ (86.4 mg, 0.27 mmol) at room temperature. The resulting mixture was stirred for 2 h at 90° C. To the above mixture was added KI (14.7 mg, 0.088 mmol) at room temperature. The resulting mixture was stirred for additional 2 h at 90° C. The resulting mixture was cooled down to room temperature and purified by reversed phase flash chromatography with the following conditions: Column: Spherical C¹⁸ Column, 20-40 um, 120 g; Mobile Phase A: water (plus 0.05% NH₄HCO₃), Mobile Phase B: ACN; Flow rate: 60 mL/min; Gradient: 30% B to 50% B in 25 min; detector: UV 220/254 nm. The fractions containing the desired product were collected at 40% B and concentrated under reduced pressure to afford (2S,4R)-1-[(2S)-2-[(1-fluorocyclopropyl)formamido]-3,3-dimethylbutanoyl]-4-hydroxy-N-[[4-(4-methyl-1,3-thiazol-5-yl)-2-(4-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]butoxy)phenyl]methyl]pyrrolidine-2-carboxamide (20.0 mg, 21%) as a white solid. ¹H NMR (400 MHz, CD₃OD) δ 8.88 (s, 1H), 8.76 (s, 1H), 7.62 (d, J=7.9 Hz, 2H), 7.48 (d, J=8.0 Hz, 1H), 7.44 (s, 1H), 7.40 (d, J=8.0 Hz, 2H), 7.03 (s, 1H), 7.01 (s, 1H), 4.75 (s, 1H), 4.70-4.40 (m, 5H), 4.12 (s, 2H), 3.86-3.70 (m, 5H), 3.57 (s, 2H), 2.67-2.53 (m, 10H), 2.50 (s, 3H), 2.29-2.20 (m, 1H), 2.19-2.02 (m, 7H), 1.95-1.85 (m, 2H), 1.83-1.73 (m, 2H), 1.60-1.50 (m, 2H), 1.31 (s, 6H), 1.03 (s, 9H). LC/MS (ESI, m/z): [(M+1)]⁺=1089.85

The following examples in Table 52 were synthesized according to the above procedure:

TABLE 52

| | | | MS: | |
|---|---|---|---|---|
| Example | I-# | Name | [(M + 1)]⁺ | ¹H NMR |
| 62 | I-212 | (2S,4R)-1-[(25)-2-[(1- | 1117.90 | (400 MHz,CD₃OD) δ 8.88 (s, 1H), |
| | | fluorocyclopropyl)formamido]- | | 8.75 (s, 1H), 7.63 (d, J = 7.9 Hz, |
| | | 3,3-dimethylbutanoyl]-4- | | 2H), 7.52-7.38 (m, 4H), 7.01 (d, |
| | | hydroxy-N-[[4-(4-methyl-1,3- | | J = 8.8 Hz, 2H), 4.76 (s, 1H), 4.65 (t, |
| | | thiazol-5-yl)-2-[(6-[4-[(4-[7- | | J = 8.4 Hz, 1H), 4.59-4.49 (m, 2H), |
| | | [trans-4-hydroxycyclohexyl]- | | 4.48-4.37 (m, 2H), 4.10 (t, J = 6.1 |
| | | 2-[(3,3,3-trifluoropropyl)ami- | | Hz, 2H), 3.90-3.78 (m, 2H), 3.76- |
| | | no]pyrrolo[2,3-d]pyrimidin-5- | | 3.70 (m, 3H), 3.63 (s, 2H), 2.91- |
| | | yl]phenyl)methyl]piperazin- | | 2.55 (m, 11H), 2.50 (s, 3H), 2.30- |
| | | 1-yl]hexyl)oxy]phenyl]meth- | | 2.20 (m, 1H), 2.17-2.04 (m, 7H), |
| | | yl]pyrrolidine-2-carboxamide | | 1.87 (d, J = 7.0 Hz, 2H), 1.70-1.52 |
| | | | | (m, 6H), 1.47 (d, J = I3 Hz, 2H), |
| | | | | 1.40-1.25 (m, 5H), 1.04 (s, 9H) |
| 63 | I-213 | (2S,4R)-1-[(2S)-2-[(1- | 1131.55 | (400 MHz, DMSO-d₆) δ 8.98 (s, |
| | | fluorocyclopropyl)formamido]- | | 1H), 8.87 (s, 1H), 8.48 (t, J = 6.0 |
| | | 3,3-dimethylbutanoyl]-4- | | Hz, 1H), 7.63 (m, 3H), 7.40 (d, J = |
| | | hydroxy-N-[[4-(4-methyl-1,3- | | 7.8 Hz, 1H), 7.34-7.25 (m, 3H), |
| | | thiazol-5-yl)-2-[(7-[4-[(4-[7- | | 7.04 (t, J = 5.9 Hz, 1H), 7.00 (d, |
| | | [trans-4-hydroxycyclohexyl]- | | J = 1.6 Hz, 1H), 6.95 (dd, J = I.I, 1.6 |
| | | 2-[(3,3,3-trifluoropropyl)ami- | | Hz, 1H), 5.17 (s, 1H), 4.69 (s, 1H), |
| | | no]pyrrolo[2,3-d]pyrimidin-5- | | 4.60 (d, J = 9.1 Hz, 1H), 4.52 (t, |
| | | yl]phenyl)methyl]piperazin-1- | | J = 8.2 Hz, 1H), 4.42 (s, 1H), 4.36 (s, |
| | | yl]heptyl)oxy]phenyl]meth- | | 1H), 4.30 (dd, J = 16.5, 6.1 Hz, |
| | | yl]pyrrolidine-2-carboxamide | | 1H), 4.20 (dd, J = 16.4, 5.6 Hz, |
| | | | | 1H), 4.05 (t, J = 6.3 Hz, 2H), 3.69- |
| | | | | 3.54 (m, 5H), 3.45 (s, 2H), 2.70- |
| | | | | 2.50 (m, 3H), 2.46 (s, 3H), 2.49- |
| | | | | 2.30 (m, 6H), 2.26 (t, J = 7.3 Hz, |
| | | | | 2H), 2.09 (t, J = 10.3 Hz, 1H), 1.97 |
| | | | | (d, J = 12.3 Hz, 4H), 1.92 (q, J = |
| | | | | 8.6, 6.1 Hz, 3H), 1.75 (t, J = 7.2 Hz, |
| | | | | 2H), 1.48-1.36 (m, 6H), 1.38-1.27 |
| | | | | (m, 7H), 1.22 (dd, J = 8.8, 3.9 Hz, |
| | | | | 2H), 0.96 (s, 9H) |
| 64 | I-174 | (2S,4R)-1-[(2S)-2-[(1- | 1105.90 | (400 MHz, CD₃OD) δ 8.87 (s, 1H), |
| | | fluorocyclopropyl)formamido]- | | 8.76 (s, 1H), 7.61 (d, J = 7.9 Hz, |
| | | 3,3-dimethylbutanoyl]-4- | | 2H), 7.51-7.39 (m, 2H), 7.39 (d, |
| | | hydroxy-N-[[4-(4-methyl-1,3- | | J = 7.9 Hz, 2H), 7.06 (d, J = 1.6 Hz, |
| | | thiazol-5-yl)-2-[2-(2-[4-[(4-[7- | | 1H), 7.04-6.98 (m, 1H), 4.76 (s, |
| | | [trans-4-hydroxycyclohexyl]- | | 1H), 4.70-4.40 (m, 8H), 4.26 (s, |
| | | 2-[(3,3,3-trifluoropropyl)ami- | | 2H), 3.92-3.68 (m, 8H), 3.62 (s, |
| | | no]pyrrolo[2,3-d]pyrimidin-5- | | 1H), 3.56 (s, 2H), 2.69-2.54 (m, |
| | | yl]phenyl)methyl]piperazin-1- | | 9H), 2.49 (s, 3H), 2.28-2.19 (m, |
| | | yl]ethoxy)ethoxy]phenyl]meth- | | 1H), 2.19-2.01 (m, 7H), 1.56 (q, |
| | | yl]pyrrolidine-2-carboxamide | | J = 10.0 Hz, 2H), 1.40-1.22 (m, 4H), |
| | | | | 1.04 (s, 9H) |
| 65 | 1-214 | (2S,4R)-1-[(2S)-2-[(1- | 1149.55 | (400 MHz, CD₃OD) δ 8.86 (s, 1H), |
| | | fluorocyclopropyl)formamido]- | | 8.74 (s, 1H), 7.62-7.55 (m, 2H), |
| | | 3,3-dimethylbutanoyl]-4- | | 7.51-7.40 (m, 2H), 7.36 (d, J = 8.0 |
| | | hydroxy-N-[[4-(4-methyl-1,3- | | Hz, 2H), 7.10-6.97 (m, 2H), 4.75 |
| | | thiazol-5-yl)-2-[2-[2-(2-[4-[(4- | | (d, J = 1.3 Hz, 1H), 4.68-4.49 (m, |
| | | [7-[trans-4-hydroxycyclohexyl]-2- | | 2H), 4.52-4.37 (m, 3H), 4.24 (q, |
| | | [(3,3,3-trifluoropropyl)ami- | | J = 4.1 Hz, 2H), 3.92 (t, J = 4.5 Hz, |
| | | no]pyrrolo[2,3-d]pyrimidin-5- | | 2H), 3.89-3.80 (m, 2H), 3.78-3.69 |
| | | yl]phenyl)methyl]piperazin-1- | | (m, 6H), 3.68-3.63 (m, 4H), 3.53 (s, |
| | | yl]ethoxy)ethoxy]ethoxy]phe- | | 2H), 2.70-2.50 (m, 11H), 2.49 (s, |
| | | nyl]methyl]pyrrolidine-2- | | 3H), 2.28-2.20 (m, 1H), 2.19-2.05 |
| | | carboxamide | | (m, 7H), 1.61-1.50 (m, 2H), 1.41- |
| | | | | 1.25 (m, 4H), 1.04 (s, 9H) |
| 66 | I-190 | (2S,4R)-1-[(25)-2-[(1- | 1193.95 | (400 MHz, CD₃OD) δ 8.87 (s, 1H), |
| | | fluorocyclopropyl)formamido]- | | 8.74 (s, 1H), 7.59 (d, J = 7.9 Hz, |
| | | 3,3-dimethylbutanoyl]-4- | | 2H), 7.50-7.40 (m, 2H), 7.37 (d, |
| | | hydroxy-N-[[4-(4-methyl-1,3- | | J = 7.9 Hz, 2H), 7.07-6.98 (m, 2H), |
| | | thiazol-5-yl)-2-(2-[2-[2-(2-[4- | | 4.78 (s, 1H), 4.68-4.37 (m, 5H), |
| | | [(4-[7-[(1r,4r)-4-hydroxycyclo- | | 4.23-4.16 (m, 2H), 3.88 (dd, J = |
| | | hexyl]-2-[(3,3,3-trifluoropro- | | 5.6, 3.4 Hz, 2H), 3.85-3.60 (m, |
| | | pyl)amino]pyrrolo[2,3-d]pyrimidin- | | 15H), 3.54 (s, 2H), 2.70-2.50 (m, |
| | | 5-yl]phenyl)methyl]piperazin-1- | | 11H), 2.49 (s, 3H), 2.30-2.20 (m, |
| | | yl]ethoxy)ethoxy]ethoxy]eth- | | 1H), 2.18-2.03 (m, 7H), 1.58-1.46 |
| | | oxy)phe-nyl]methyl]pyrrolidine- | | (m, 2H), 1.42-1.26 (m, 5H), 1.04 (s, |
| | | 2-carboxamide | | 9H) |

### EXAMPLE 67: 3-[6-([2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]ethyl]amino)pyrido[2,3-b]indol-9-yl]piperidine-2,6-dione; formic acid (I-133)

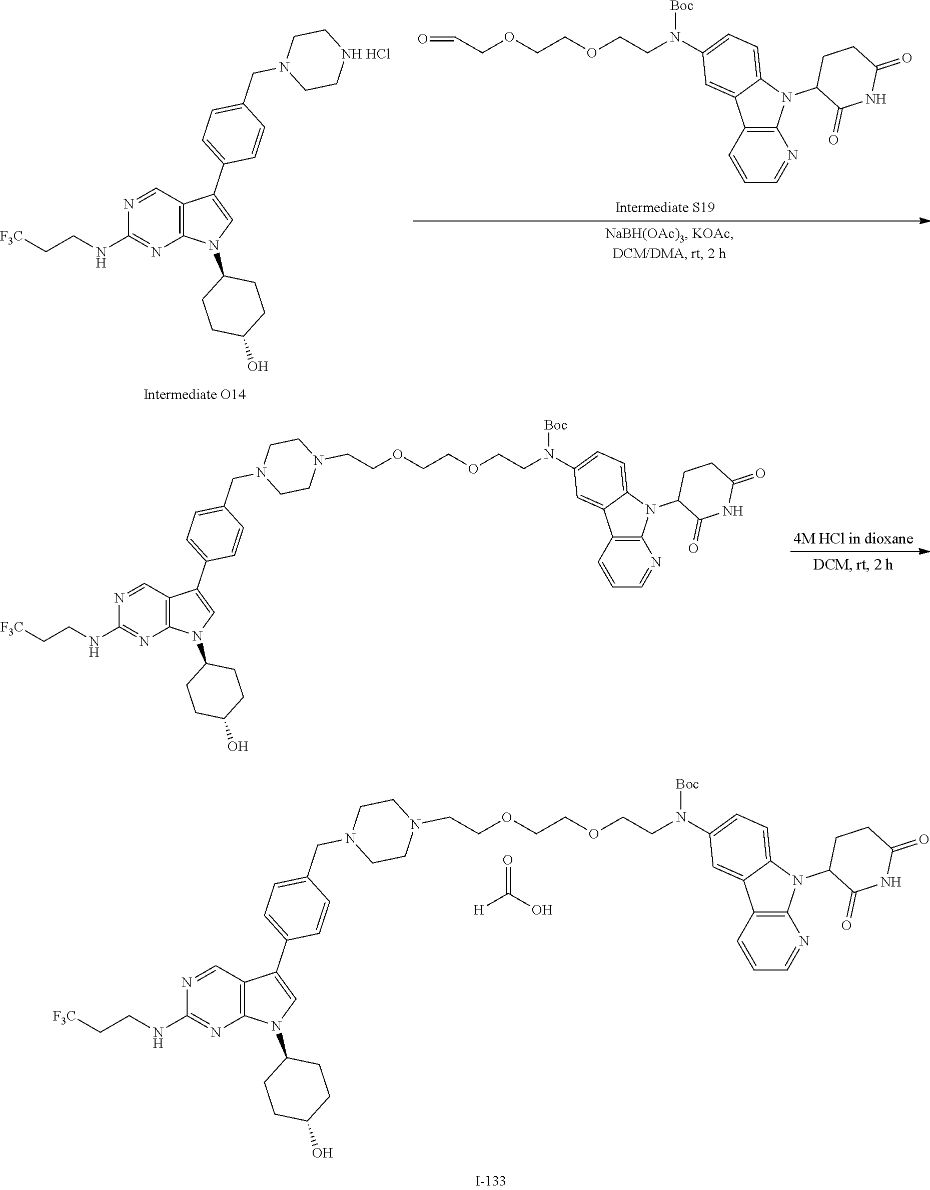

### Step 1: tert-butyl N-[9-(2,6-dioxopiperidin-3-yl)pyrido[2,3-b]indol-6-yl]-N-[2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]ethyl]carbamate

To a stirred solution of trans-4-[5-[4-(piperazin-1-ylmethyl)phenyl]-2-[(3,3,3-trifluoropropyl)amino]pyrrolo[2,3-d]pyrimidin-7-yl]cyclohexan-1-ol hydrochloride (37.0 mg, 0.074 mmol) and 2-[2-(2-[[9-(2,6-dioxopiperidin-3-yl)pyrido[2,3-b]indol-6-yl]amino]ethoxy)ethoxy]acetaldehyde (38.6 mg, 0.074 mmol) in DCM (10.0 mL) was added KOAc (28.9 mg, 0.29 mmol) at room temperature. Then NaBH(OAc)₃ (46.8 mg, 0.221 mmol) was added to the reaction. The reaction was stirred for 1 hour at room temperature. Upon completion, the solution was concentrated under reduced pressure and purified by reversed phase flash chromatograph with the following conditions: Column: Xselect CSH OBD Column 30×150 mm 5 um; Mobile Phase A: water (plus 0.1% FA), Mobile Phase B: ACN; Flow rate: 60 mL/min; Gradient: 13% B to 22% B in 7 min; detector: 220/254 nm; desired fractions were collected at 6.48 min and lyophilized to afford tert-butyl N-[9-(2,6-dioxopiperidin-3-yl)pyrido[2,3-b]indol-6-yl]-N-[2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]ethyl]carbamate (20.0 mg, 30%) as a white solid. ¹H NMR (400 MHz, DMSO-d₆) δ 11.18 (s, 1H), 8.87 (s, 1H), 8.57 (dd, J=7.6, 1.7 Hz, 1H), 8.43 (dd, J=4.9, 1.6 Hz, 1H), 8.13 (d, J=2.1 Hz, 1H), 7.66-7.53 (m, 3H), 7.39 (d, J=8.7 Hz, 1H), 7.32-7.16 (m, 2H), 7.05 (t, J=5.8 Hz, 1H), 6.94-6.85 (s, 1H), 6.04 (s, 1H), 4.70 (d, J=4.5 Hz, 1H), 4.50-4.37 (m, 1H), 3.86-3.63 (m, 3H), 3.62-3.35 (m, 13H), 3.18-2.94 (m, 2H), 2.75-2.55 (m, 2H), 2.50-2.20 (m, 13H), 2.15-2.04 (m, 1H), 2.04-1.53 (m, 6H), 1.31 (m, 9H). LC/MS (ESI, m/z): [(M+1)]⁺=1011.50.

The following examples in Table 53 were synthesized according to the above procedure:

TABLE 53

| | | | MS: | |
|---|---|---|---|---|
| Example | Structure | Name | [(M + 1)]⁺ | ¹H NMR |
| 68 | I-55 | 3-[3-methyl-2-oxo-5-(3- | 934.50 | (400 MHz, CD₃OD) δ 8.75 (s, 1H), |
| | | [2-[2-(2-[4-[(4-[7-[trans- | | 7.60 (d, J = 7.8 Hz, 2H), 7.44 (s, |
| | | 4-hydroxycyclohexyl]-2- | | 1H), 7.37 (d, J = 7.7 Hz, 2H), 7.09- |
| | | [(3,3,3- | | 6.86 (m, 3H), 5.31 (dd, J = 12.7, 5.4 |
| | | trifluoropropyl)amino]- | | Hz, 1H), 4.63 (s, 1H), 4.58-4.48 (m, |
| | | 7H-pyrrolo[2,3- | | 1H), 3.80-3.52 (m, 16H), 3.45 (t, J = |
| | | d]pyrimidin-5- | | 6.3 Hz, 2H), 3.40 (s, 3H), 2.98- |
| | | yl]phenyl)methyl]piperazin- | | 2.85 (m, 1H), 2.85-2.46 (m, 14H), |
| | | 1- | | 2.18-2.00 (m, 7H), 1.87 (t, J = 7.3 |
| | | yl]ethoxy)ethoxy]ethoxy] | | Hz, 2H), 1.54 (t, J = 9.6 Hz, 2H). |
| | | propyl)-2,3-dihydro-1H- | | |
| | | 1,3-benzodiazol-1- | | |
| | | yl]piperidine-2,6-dione | | |
| 69 | I-78 | 3-(3-methyl-2-oxo-5-[3- | [M/2 + | (400 MHz, CD₃OD) δ 8.74 (s, 1H), |
| | | [3-(2-[4-[(4-[7-[trans-4- | 1)]⁺ = | 8.52 (s, 1H), 7.63 (d, J = 7.8 Hz, |
| | | hydroxycyclohexyl]-2- | 452.90 | 2H), 7.46-7.37 (m, 3H), 7.00 (d, J = |
| | | [(3,3,3- | | 11.5 Hz, 2H), 6.94 (d, J = 8.1 Hz, |
| | | trifluoropropyl)amino]- | | 1H), 5.31 (dd, J = 12.6, 5.4 Hz, |
| | | 7H-pyrrolo[2,3- | | 1H), 4.80-4.50 (m, 7H), 3.74-3.62 |
| | | d]pyrimidin-5- | | (m, 6H), 3.60-3.50 (m, 4H), 3.48- |
| | | yl]phenyl)methyl]piperazin- | | 3.39 (m, 5H), 2.95-2.83 (m, 5H), |
| | | 1- | | 2.78-2.69 (m, 5H), 2.58 (dq, J = |
| | | yl]ethoxy)propoxy]propyl]- | | 11.5, 5.6, 4.0 Hz, 2H), 2.17-2.07 |
| | | 2,3-dihydro-1H-1,3- | | (m, 3H), 2.07-2.01 (m, 4H), 1.93- |
| | | benzodiazol-1- | | 1.80 (m, 4H), 1.58-1.50 (m, 2H) |
| | | yl)piperidine-2,6-dione; | | |
| | | formic acid | | |
| 70 | I-56 | 3-(3-methyl-2-oxo-5-[3- | 890.40 | (400 MHz, CD₃OD) δ 8.74 (s, 1H), |
| | | [2-(2-[4-[(4-[trans-4- | | 7.61-7.55 (m, 2H), 7.43 (s, 1H), |
| | | hydroxycyclohexyl]-2- | | 7.39-7.33 (m, 2H), 7.06-6.93 (m, |
| | | [(3,3,3- | | 3H), 5.31 (m, 1H), 4.55 (q, J = 8.5, |
| | | trifluoropropyl)amino]- | | 7.8 Hz, 1H), 3.76-3.57 (m, 9H), |
| | | 7H-pyrrolo[2,3- | | 3.55 (s, 2H), 3.48 (t, J = 6.3 Hz, |
| | | d]pyrimidin-5- | | 2H), 3.41 (s, 3H), 2.96-2.85 (m, |
| | | yl]phenyl)methyl] | | 1H), 2.85-2.71 (m, 5H), 2.71-2.33 |
| | | piperazin-1- | | (m, 11H), 2.19-2.03 (m, 7H), 1.96- |
| | | yl]ethoxy)ethoxy]propyl]- | | 1.84 (m, 2H), 1.54 (m, 2H) |
| | | 2,3-dihydro-1H-1,3- | | |
| | | benzodiazol-1- | | |
| | | yl)piperidine-2,6-dione | | |
| 71 | I-82 | 3-(3-methyl-2-oxo-5-[3- | [(M/2 + | (400 MHz, CD₃OD) δ 8.75 (d, J = |
| | | [2-(2-[4-[(4-[7-[trans-4- | 1)]⁺= | 2.5 Hz, 1H), 7.60 (dd, J = 8.1, 6.0 |
| | | hydroxycyclohexyl]-2- | 443.90 | Hz, 2H), 7.44 (d, J = 1.8 Hz, 1H), |
| | | [(3,3,3- | | 7.36 (dd, J = 8.1, 6.2 Hz, 2H), 7.29- |
| | | trifluoropropyl)amino]- | | 6.99 (m, 3H), 5.38-5.30 (m, 1H), |
| | | 7H-pyrrolo[2,3- | | 4.62-4.50 (m, 1H), 4.45-4.29 (m, |
| | | d]pyrimidin-5- | | 2H), 3.82-3.60 (m, 9H), 3.54 (d, J = |
| | | yl]phenyl)methyl] | | 4.2 Hz, 2H), 3.45-3.42 (m, 3H), |
| | | piperazin-1- | | 3.02-2.71 (m, 3H), 2.70-2.26 (m, |
| | | yl]ethoxy)ethoxy]prop-1- | | 10H), 2.19-1.99 (m, 7H), 1.63-1.48 |
| | | yn-1-yl]-2,3-dihydro-1H- | | (m, 2H) |
| | | 1,3-benzodiazol-1- | | |
| | | yl)piperidine-2,6-dione | | |
| 72 | I-63 | 3-[3-methyl-2-oxo-5-(3- | 935.45 | (400 MHz, CD₃OD) δ 8.99 (s, 1H), |
| | | [2-[2-(2-[4-[(4-[1-[trans- | | 8.50 (s, 1H), 7.92 (d, J = 8.1 Hz, |
| | | 4-hydroxycyclohexyl]-6- | | 2H), 7.46 (d, J = 8.1 Hz, 2H), 7.04- |
| | | [(3,3,3- | | 6.96 (m, 2H), 6.94 (dd, J = 8.0, 1.5 |
| | | trifluoropropyl)amino]- | | Hz, 1H), 5.34-5.29 (m, 1H), 4.70- |
| | | 1H-pyrazolo[3,4- | | 4.59 (m, 1H), 3.79-3.70 (m, 5H), |
| | | d]pyrimidin-3- | | 3.69-3.62 (m, 9H), 3.58 (dd, J = |
| | | yl]phenyl)methyl] | | 5.8, 2.9 Hz, 2H), 3.45 (t, J = 6.4 Hz, |
| | | piperazin-1- | | 2H), 3.40 (s, 3H), 3.10-3.02 (m, |
| | | yl]ethoxy)ethoxy)ethoxy] | | 5H), 2.94-2.87 (m, 1H), 2.86-2.55 |
| | | propyl)-2,3-dihydro-1H- | | (m, 10H), 2.31-2.20 (m, 2H), 2.18- |
| | | 1,3-benzodiazol-1- | | 2.09 (m, 3H), 2.08-2.02 (m, 2H), |
| | | yl]piperidine-2,6-dione; | | 1.93-1.81 (m, 2H), 1.60-1.47 (m, |
| | | formic acid | | 2H) |
| 73 | I-64 | 3-(3-methyl-2-oxo-5-[3- | 891.50 | (400 MHz, CD₃OD) δ 8.99 (s, 1H), |
| | | [2-(2-[4-[(4-[1-[trans-4- | | 7.93-7.86 (m, 2H), 7.46 (d, J = 8.0 |
| | | hydroxycyclohexyl]-6- | | Hz, 2H), 7.06-6.98 (m, 2H), 6.96 |
| | | [(3,3,3- | | (dd, J = 8.1, 1.5 Hz, 1H), 5.35-5.29 |
| | | trifluoropropyl)amino]- | | (m, 1H), 4.71-4.60 (m, 1H), 3.78- |
| | | 1H-pyrazolo[3,4- | | 3.68 (m, 5H), 3.67-3.57 (m, 6H), |
| | | d]pyrimidin-3- | | 3.50 (t, J = 6.3 Hz, 2H), 3.41 (s, |
| | | yl]phenyl)methyl] | | 3H), 3.02-2.84 (m, 6H), 2.83-2.72 |
| | | piperazin-1- | | (m, 5H), 2.70-2.53 (m, 6H), 2.38- |
| | | yl]ethoxy)ethoxy]propyl]- | | 2.22 (m, 2H), 2.20-2.11 (m, 3H), |
| | | 2,3-dihydro-1H-1,3- | | 2.10-2.02 (m, 2H), 1.96-1.86 (m, |
| | | benzodiazol-1- | | 2H), 1.60-1.50 (m, 2H) |
| | | yl)piperidine-2,6-dione | | |
| 74 | I-49 | 3-(5-[3-[2-(2-[2-[4-([4- | 895.50 | (400 MHz, CD₃OD) δ 8.94 (s, 1H), |
| | | [6-(butylamino)-1-[trans- | | 8.53 (s, 1H), 7.93-7.87 (m, 2H), |
| | | 4-hydroxycyclohexyl]- | | 7.49-7.42 (m, 2H), 7.04-6.97 (m, |
| | | 1H-pyrazolo[3,4- | | 2H), 6.94 (dd, J = 8.1, 1.6 Hz, 1H), |
| | | d]pyrimidin-3- | | 5.35-5.28 (m, 1H), 4.69-4.58 (m, |
| | | yl]phenyl]methyl) | | 1H), 3.78-3.70 (m, 2H), 3.68-3.62 |
| | | piperazin-1- | | (m, 9H), 3.58 (dt, J = 4.6, 3.2 Hz, |
| | | yl]ethoxy]ethoxy)ethoxy] | | 2H), 3.52-3.42 (m, 4H), 3.40 (s, |
| | | propyl]-3-methyl-2-oxo- | | 3H), 3.04-2.86 (m, 6H), 2.85-2.59 |
| | | 2,3-dihydro-1H-1,3- | | (m, 8H), 2.30-2.20 (m, 2H), 2.19- |
| | | benzodiazol-1- | | 2.10 (m, 3H), 2.08-2.00 (m, 2H), |
| | | yl)piperidine-2,6-dione; | | 1.87 (dt, J = 8.4, 6.4 Hz, 2H), 1.68 |
| | | formic acid | | (p, J = 7.2 Hz, 2H), 1.60-1.43 (m, |
| | | | | 4H), 1.40-1.28 (m, 1H), 1.03 (t, J = |
| | | | | 7.4 Hz, 3H) |
| 75 | I-50 | 3-[5-[3-(2-[2-[4-[(4-[6- | 851.55 | (400 MHz, CD₃OD) δ 8.93 (s, 1H), |
| | | (butylamino)-1-[trans-4- | | 8.53 (s, 1H), 7.88 (d, J = 8.1 Hz, |
| | | hydroxycyclohexyl]-1H- | | 2H), 7.46 (d, J = 8.0 Hz, 2H), 7.06- |
| | | pyrazolo[3,4- | | 6.98 (m, 2H), 6.96 (dd, J = 8.1, 1.5 |
| | | d]pyrimidin-3- | | Hz, 1H), 5.35-5.28 (m, 1H), 4.68- |
| | | yl]phenyl]methyl) | | 4.62 (m, 1H), 3.72 (dd, J = 6.7, 4.1 |
| | | piperazin-1- | | Hz, 2H), 3.67-3.57 (m, 7H), 3.53- |
| | | yl]ethoxy]ethoxy)propyl]- | | 3.45 (m, 4H), 3.41 (s, 3H), 3.00- |
| | | 3-methyl-2-oxo-2,3- | | 2.86 (m, 6H), 2.85-2.80 (m, 2H), |
| | | dihydro-1H-1,3- | | 2.78-2.72 (m, 3H), 2.70-2.60 (m, |
| | | benzodiazol-1- | | 3H), 2.30-2.09 (m, 5H), 2.07-1.98 |
| | | yl]piperidine-2,6-dione; | | (m, 2H), 1.91 (dq, J = 8.4, 6.5 Hz, |
| | | formic acid | | 2H), 1.69 (p, J = 7.1 Hz, 2H), 1.61- |
| | | | | 1.43 (m, 4H), 1.39-1.27 (m, 1H), |
| | | | | 1.03 (t, J = 7.4 Hz, 3H). |
| 76 | I-41 | 3-(5-[1-[4-([4-[2- | 938.60 | (400 MHz, DMSO-d₆) δ 11.11 (s, |
| | | (butylamino)-7-[trans-4- | | 1H), 8.82 (s, 1H), 7.63 (d, J = 7.9 |
| | | hydroxycyclohexyll]-7H- | | Hz, 2H), 7.58 (s, 1H), 7.30 (d, J = |
| | | pyrrolo[2,3-d]pyrimidin- | | 7.8 Hz, 2H), 7.06-6.97 (m, 2H), |
| | | 5- | | 6.90-6.83 (m, 2H), 5.39-5.32 (m, |
| | | yl]phenyl]methyl)piperazin- | | 1H), 4.71 (d, J = 4.5 Hz, 1H), 4.51- |
| | | 1-yl]-3,6,9,12- | | 4.34 (m, 1H), 3.55-3.45 (m, 17H), |
| | | tetraoxapentadecan-15- | | 3.42-3.28 (m, 13H), 2.95-2.83 (m, |
| | | yl]-3-methyl-2-oxo-2,3- | | 1H), 2.68-2.60 (m, 4H), 2.48-2.30 |
| | | dihydro-1H-1,3- | | (m, 3H), 2.03-1.94 (m, 5H), 1.93- |
| | | benzodiazol-1- | | 1.83 (m, 3H), 1.83-1.74 (m, 2H), |
| | | yl)piperidine-2,6-dione | | 1.56 (p, J = 7.2 Hz, 2H), 1.43-1.30 |
| | | | | (m, 4H), 0.93 (t, J = 7.3 Hz, 3H). |
| 77 | I-42 | 3-[5-[3-(2-[2-[4-([4-[2- | 850.45 | (400 MHz, DMSO-d₆) δ 8.38 (s, |
| | | (butylamino)-7-[trans-4- | | 2H), 8.22 (s, 1H), 7.32-7.15 (m, |
| | | hydroxycyclohexyl]-7H- | | 5H), 6.65 (s, 1H), 6.57 (s, 2H), 5.03 |
| | | pyrrolo[2,3-d]pyrimidin- | | (d, J = 8.1 Hz, 1H), 4.12-4.02 (m, |
| | | 5-yl]phenyl]methyl) | | 1H), 3.80-3.65 (m, 4H), 3.60-3.48 |
| | | piperazin-1-yl]ethoxy] | | (m, 5H), 3.31 (d, J = 7.3 Hz, 2H), |
| | | ethoxy)propyl]- | | 3.23-3.16 (m, 3H), 3.11 (s, 3H), |
| | | 3-methyl-2-oxo-2,3- | | 3.07 (s, 3H), 2.96-2.78 (m, 3H), |
| | | dihydro-1H-1,3- | | 2.77-2.60 (m, 3H), 2.40-2.30 (m, |
| | | benzodiazol-1- | | 3H), 1.94-1.82 (m, 3H), 1.70-1.40 |
| | | yl]piperidine-2,6-dione; | | (m, 8H), 1.33-1.20 (m, 5H), 0.83 (t, |
| | | formic acid | | J = 7.3 Hz, 3H). |
| 78 | I-43 | 3-[5-(3-[2-[4-([4-[2- | 806.50 | (400 MHz, DMSO-d₆) δ 11.11 (s, |
| | | (butylamino)-7-[trans-4- | | 1H), 8.82 (s, 1H), 8.16 (s, 1H), |
| | | hydroxycyclohexyl]-7H- | | 7.71-7.54 (m, 3H), 7.31 (d, J = 7.9 |
| | | pyrrolo[2,3-d]pyrimidin- | | Hz, 2H), 7.12-6.94 (m, 2H), 6.87 |
| | | 5- | | (d, J = 8.5 Hz, 2H), 5.34 (dd, J = |
| | | yl]phenyl]methyl)piperazin- | | 12.8, 5.4 Hz, 1H), 4.54-4.34 (m, |
| | | 1-yl]ethoxy]propyl)-3- | | 1H), 3.62-3.42 (m, 6H), 3.37 (q, J = |
| | | methyl-2-oxo-2,3- | | 6.3 Hz, 2H), 3.30 (s, 3H), 2.97- |
| | | dihydro-1H-1,3- | | 2.82 (m, 1H), 2.77-2.51 (m, 8H), |
| | | benzodiazol-1- | | 2.49-2.40 (m, 6H), 2.03-1.85 (m, |
| | | yl]piperidine-2,6-dione; | | 8H), 1.80 (q, J = 7.0, 6.6 Hz, 2H), |
| | | formic acid | | 1.56 (p, J = 7.3 Hz, 2H), 1.42-1.30 |
| | | | | (m, 4H), 0.93 (t, J = 7.4 Hz, 3H). |
| 79 | I-44 | 3-(5-[3-[4-([4-[2- | 762.50 | (400 MHz, DMSO-d₆) δ 11.10 (s, |
| | | (Butylamino)-7-[trans-4- | | 1H), 8.83 (s, 1H), 8.15 (s, 1H), 7.63 |
| | | hydroxycyclohexyl]-7H- | | (d, J = 7.9 Hz, 2H), 7.58 (s, 1H), |
| | | pyrrolo[2,3-d]pyrimidin- | | 7.31 (d, J = 7.9 Hz, 2H), 7.05 (d, J = |
| | | 5- | | 1.5 Hz, 1H), 7.00 (d, J = 8.0 Hz, |
| | | yl]phenyl]methyl)piperazin- | | 1H), 6.86 (dd, J = 11.5, 6.8 Hz, |
| | | 1-yl]propyl]-3-methyl- | | 2H), 5.34 (dd, J = 12.7, 5.4 Hz, |
| | | 2-oxo-2,3-dihydro-1H- | | 1H), 4.71 (s, 1H), 4.41 (d, J = 12.7 |
| | | 1,3-benzodiazol-1- | | Hz, 1H), 3.56-3.25 (m, 9H), 2.97- |
| | | yl)piperidine-2,6-dione; | | 2.83 (m, 1H), 2.76-2.67 (m, 1H), |
| | | formic acid | | 2.66-2.58 (m, 3H), 2.51-2.33 (m, |
| | | | | 8H), 2.03-1.93 (m, 5H), 1.92-1.83 |
| | | | | (m, 3H), 1.77 (d, J = 9.5 Hz, 2H), |
| | | | | 1.56 (p, J = 7.2 Hz, 2H), 1.42-1.31 |
| | | | | (m, 4H), 0.93 (t, J = 7.3 Hz, 3H). |
| 80 | I-58 | 3-(5-[1-[4-([4-[2- | 956.55 | (400 MHz, CDCl₃) δ 8.75 (s, 1H), |
| | | (butylamino)-7-[trans-4- | | 7.53 (t, J = 7.8 Hz, 1H), 7.20-7.14 |
| | | hydroxycyclohexyl]-7H- | | (m, 3H), 6.92 (d, J = 8.8 Hz, 2H), |
| | | pyrrolo[2,3-d]pyrimidin- | | 6.75 (d, J = 7.9 Hz, 1H), 6.05 (s, |
| | | 5-yl]-3- | | 1H), 5.25 (dd, J = 12.8, 5.3 Hz, |
| | | fluorophenyl]methyl)piperazin- | | 1H), 4.62-4.52 (m, 1H), 3.84-3.72 |
| | | 1-yl]-3,6,9,12- | | (m, 3H), 3.70-3.55 (m, 14H), 3.55- |
| | | tetraoxapentadecan-15- | | 3.43 (m, 7H), 3.00-2.79 (m, 7H), |
| | | yl]-3-methyl-2-oxo-2,3- | | 2.79-2.70 (m, 7H), 2.26-2.17 (m, |
| | | dihydro-1H-1,3- | | 5H), 1.94-1.91 (m, 5H), 1.75-1.53 |
| | | benzodiazol-1- | | (m, 5H), 1.49 (m, J = 7.5 Hz, 2H), |
| | | yl)piperidine-2,6-dione | | 1.01 (t, J = 7.3 Hz, 3H). |
| 81 | I-57 | 3-(5-[1-[4-([4-[2- | 922.55 | (400 MHz, CD₃OD) δ 8.70 (s, 1H), |
| | | (butylamino)-7- | | 7.60 (d, J = 7.7 Hz, 2H), 7.46-7.29 |
| | | cyclohexyl-7H- | | (m, 3H), 7.06-6.89 (m, 3H), 5.37- |
| | | pyrrolo[2,3-d]pyrimidin- | | 5.23 (m, 1H), 4.70-4.60 (m, 1H), |
| | | 5- | | 4.58-4.48 (m, 1H), 3.67-3.51 (m, |
| | | yl]phenyl]methyl)piperazin- | | 16H), 3.47-3.38 (m, 7H), 2.96-2.52 |
| | | 1-yl]-3,6,9,12- | | (m, 14H), 2.18-1.75 (m, 10H), 1.66 |
| | | tetraoxapentadecan-15- | | (p, J = 7.0 Hz, 2H), 1.58-1.28 (m, |
| | | yl]-3-methyl-2-oxo-2,3- | | 5H), 1.01 (t, J = 7.3 Hz, 3H) |
| | | dihydro-1H-1,3- | | |
| | | benzodiazol-1- | | |
| | | yl)piperidine-2,6-dione | | |
| 82 | I-59 | 3-(5-[1-[4-(1-[4-[2- | 952.55 | (400 MHz, CDCl₃) δ 8.82 (d, J = |
| | | (butylamino)-7-[trans-4- | | 1.7 Hz, 1H), 7.55-7.48 (m, 2H), |
| | | hydroxycyclohexyl]-7H- | | 7.35 (d, J = 7.8 Hz, 2H), 7.07 (d, J = |
| | | pyrrolo[2,3-d]pyrimidin- | | 2.1 Hz, 1H), 6.96-6.88 (m, 2H), |
| | | 5- | | 6.75 (d, J = 8.0 Hz, 1H), 5.88 (s, |
| | | yl]phenyl]ethyl)piperazin- | | 1H), 5.25 (m, 1H), 4.55 (d, J = 12.0 |
| | | 1-yl]-3,6,9,12- | | Hz, 1H), 3.80 (m, 1H), 3.69-3.62 |
| | | tetraoxapentadecan-15- | | (m, 10H), 3.66-3.56 (m, 4H), 3.55- |
| | | yl]-3-methyl-2-oxo-2,3- | | 3.43 (m, 8H), 2.99-2.56 (m, 14H), |
| | | dihydro-1H-1,3- | | 2.30-2.10 (m, 5H), 2.08-1.85 (m, |
| | | benzodiazol-1- | | 4H), 1.73-1.55 (m, 5H), 1.52-1.43 |
| | | yl)piperidine-2,6-dione | | (m, 5H), 1.01 (t, J = 7.3 Hz, 3H) |
| 83 | I-62 | 3-[5-[3-(3-[3-[4-([4-[2- | 878.55 | (400 MHz, CD₃OD) δ 8.80 (s, 1H), |
| | | (butylamino)-7-[trans-4- | | 7.92 (s, 1H), 7.73 (d, J = 7.8 Hz, |
| | | hydroxycyclohexyl]-7H- | | 2H), 7.59 (d, J = 7.8 Hz, 2H), 7.05 |
| | | pyrrolo[2,3-d]pyrimidin- | | (t, J = 4.1 Hz, 2H), 6.97 (d, J = 8.3 |
| | | 5- | | Hz, 1H), 5.40-5.30 (m, 1H), 4.70- |
| | | yl]phenyl]methyl)piperazin- | | 4.60 (m, 1H), 4.12-4.00 (m, 2H), |
| | | 1- | | 3.80-3.70 (m, 1H), 3.62-3.50 (m, |
| | | yl]propoxy]propoxy) | | 10H), 3.50-3.35 (m, 6H), 3.30-3.23 |
| | | propyl]-3-methyl-2-oxo-2,3- | | (m, 3H), 2.96-2.89 (m, 1H), 2.87- |
| | | dihydro-1H-1,3- | | 2.73 (m, 5H), 2.22-2.02 (m, 10H), |
| | | benzodiazol-1- | | 2.00-1.80 (m, 5H), 1.73 (q, J = 7.2 |
| | | yl]piperidine-2,6-dione | | Hz, 2H), 1.63-1.46 (m, 5H), 1.05 (t, |
| | | dihydrochloride | | J = 7.4 Hz, 3H). |
| 84 | I-69 | 3-[5-(3-[3-[4-([4-[2- | 820.35 | (400 MHz, CD₃OD) δ 8.71 (s, 1H), |
| | | (butylamino)-7-[trans-4- | | 8.48 (s, 2H), 7.63 (d, J = 7.8 Hz, |
| | | hydroxycyclohexyl]-7H- | | 2H), 7.43-7.29 (m, 3H), 7.07-7.00 |
| | | pyrrolo[2,3-d]pyrimidin- | | (m, 2H), 6.97 (d, J = 8.0 Hz, 1H), |
| | | 5- | | 5.32 (dd, J = 12.6, 5.3 Hz, 1H), |
| | | yl]phenyl]methyl)piperazin- | | 4.60-4.52 (m, 1H), 3.78-3.69 (m, |
| | | 1-yl]propoxy]propyl)- | | 3H), 3.58-3.38 (m, 10H), 3.10-2.86 |
| | | 3-methyl-2-oxo-2,3- | | (m, 7H), 2.85-2.56 (m, 8H), 2.22- |
| | | dihydro-1H-1,3- | | 2.11 (m, 3H), 2.10-2.01 (m, 4H), |
| | | benzodiazol-1- | | 1.96-1.86 (m, 4H), 1.68 (p, J = 7.3 |
| | | yl]piperidine-2,6-dione; | | Hz, 2H), 1.60-1.43 (m, 4H), 1.02 (t, |
| | | bis(formic acid) | | J = 7.3 Hz, 3H) |
| 85 | I-70 | 3-(5-[3-[3-(2-[4-[(4-[2- | 876.50 | (400 MHz, CD₃OD) δ 8.70 (s, 1H), |
| | | [(2- | | 7.60 (d, J = 7.9 Hz, 2H), 7.42-7.35 |
| | | cyclopropylethyl)amino]- | | (m, 3H), 7.04-6.97 (m, 2H), 6.94 |
| | | 7-[trans-4- | | (d, J = 8.3 Hz, 1H), 5.31 (dd, J = |
| | | hydroxycyclohexyl]-7H- | | 12.7, 5.4 Hz, 1H), 4.60-4.50 (m, |
| | | pyrrolo[2,3-d]pyrimidin- | | 1H), 3.78-3.68 (m, 1H), 3.63-3.49 |
| | | 5- | | (m, 10H), 3.47-3.38 (m, 6H), 2.95- |
| | | yl]phenyl)methyl]piperazin- | | 2.86 (m, 1H), 2.82-2.50 (m, 13H), |
| | | 1- | | 2.20-2.10 (m, 3H), 2.09-2.02 (m, |
| | | yl]ethoxy)propoxy] | | 4H), 1.94-1.80 (m, 4H), 1.62-1.48 |
| | | propyl]-3-methyl-2-oxo-2,3- | | (m, 4H), 0.86-0.77 (m, 1H), 0.51 |
| | | dihydro-1H-1,3- | | (d, J = 7.7 Hz, 2H), 0.15 (d, J = 5.1 |
| | | benzodiazol-1- | | Hz, 2H). |
| | | yl)piperidine-2,6-dione | | |
| 86 | I-72 | 3-(5-[3-[3-(2-[4-[(4-[2- | 862.50 | (400 MHz, CD₃OD) δ 8.70 (s, 1H), |
| | | [(cyclopropylmethyl) | | 8.52 (s, 1H), 7.62 (d, J = 7.8 Hz, |
| | | amino]-7-(trans-4- | | 2H), 7.44-7.37 (m, 3H), 7.07-6.97 |
| | | hydroxycyclohexyl]-7H- | | (m, 2H), 6.93 (d, J = 8.1 Hz, 1H), |
| | | pyrrolo[2,3-d]pyrimidin- | | 5.30 (dd, J = 12.7, 5.5 Hz, 1H), 4.55 |
| | | 5- | | (dt, J = 10.2, 5.0 Hz, 1H), 3.78-3.62 |
| | | yl]phenyl)methyl]piperazin- | | (m, 5H), 3.60-3.48 (m, 4H), 3.45- |
| | | 1- | | 3.38 (m, 4H), 3.37 (s, 3H), 2.99- |
| | | yl]ethoxy)propoxy]propyl]- | | 2.85 (m, 7H), 2.84-2.49 (m, 8H), |
| | | 3-methyl-2-oxo-2,3- | | 2.20-2.08 (m, 3H), 2.08-1.94 (m, |
| | | dihydro-1H-1,3- | | 4H), 1.92-1.82 (m, 4H), 1.54 (qd, J = |
| | | benzodiazol-1- | | 11.9, 5.3 Hz, 2H), 1.17 (ddt, J = |
| | | yl)piperidine-2,6-dione; | | 10.6, 7.8, 3.9 Hz, 1H), 0.59-0.50 |
| | | formic acid | | (m, 2H), 0.33 (t, J = 5.0 Hz, 2H). |
| 87 | I-71 | 3-[3-methyl-2-oxo-5-[3- | 850.50 | (400 MHz, CD₃OD) δ 8.70 (s, 1H), |
| | | (3-[2-[4-([4-[2- | | 7.60 (d, J = 7.7 Hz, 2H), 7.40 (s, |
| | | (propylamino)-7-[trans- | | 1H), 7.38 (d, J = 8.4 Hz, 2H), 7.04- |
| | | 4-hydroxycyclohexyl]- | | 6.97 (m, 2H), 6.94 (d, J = 8.3 Hz, |
| | | 7H-pyrrolo[2,3- | | 1H), 5.31 (dd, J = 12.6, 5.5 Hz, |
| | | d]pyrimidin-5- | | 1H), 4.60-4.50 (m, 1H), 3.80-3.36 |
| | | yl]phenyl]methyl)piperazin- | | (m, 17H), 2.95-2.86 (m, 1H), 2.85- |
| | | 1- | | 2.71 (m, 13H), 2.18-2.09 (m, 3H), |
| | | yl]ethoxy]propoxy)propyl]- | | 2.08-2.02 (m, 4H), 1.92-1.79 (m, |
| | | 2,3-dihydro-1H-1,3- | | 4H), 1.70 (q, J = 7.2 Hz, 2H), 1.60- |
| | | benzodiazol-1- | | 1.48 (m, 2H), 1.03 (t, J = 7.4 Hz, |
| | | yl]piperidine-2,6-dione | | 3H). |
| 88 | I-73 | 3-[3-methyl-2-oxo-5-[3- | 860.50 | (400 MHz, CD₃OD) δ 8.75 (s, 1H), |
| | | (3-[4-[(4-[7-[trans-4- | | 8.51 (s, 1H), 7.63 (d, J = 7.8 Hz, |
| | | hydroxycyclohexyl]-2- | | 2H), 7.54-7.34 (m, 3H), 7.12-6.99 |
| | | [(3,3,3- | | (m, 2H), 6.99-6.93 (m, 1H), 5.32 |
| | | trifluoropropyl)amino]- | | (dd, J = 12.4, 5.3 Hz, 1H), 4.62- |
| | | 7H-pyrrolo[2,3- | | 4.52 (m, 1H), 3.79-3.66 (m, 5H), |
| | | d]pyrimidin-5- | | 3.53-3.43 (m, 4H), 3.42 (s, 3H), |
| | | yl]phenyl)methyl]piperazin- | | 3.04-2.53 (m, 17H), 2.24-2.01 (m, |
| | | 1-yl]propoxy)propyl]- | | 7H), 1.98-1.89 (m, 4H), 1.54 (p, J = |
| | | 2,3-dihydro-1H-1,3- | | 10.5 Hz, 2H). |
| | | benzodiazol-1- | | |
| | | yl]piperidine-2,6-dione- | | |
| | | formic acid | | |
| 89 | I-74 | 3-[5-(3-[3-[4-([4-[2- | 804.35 | (400 MHz, CD₃OD) δ 8.71 (s, 1H), |
| | | (butylamino)-7- | | 7.61 (d, J = 8.1 Hz, 2H), 7.44-7.36 |
| | | cyclohexyl-7H- | | (m, 3H), 7.07-6.99 (m, 2H), 6.97 |
| | | pyrrolo[2,3-d]pyrimidin- | | (dd, J = 8.1, 1.5 Hz, 1H), 5.36-5.30 |
| | | 5- | | (m, 1H), 4.60-4.50 (m, 1H), 3.58 (s, |
| | | yl]phenyl]methyl)piperazin- | | 2H), 3.51-3.41 (m, 9H), 2.97-2.87 |
| | | 1-yl]propoxy]propyl)- | | (m, 1H), 2.86-2.74 (m, 4H), 2.72- |
| | | 3-methyl-2-oxo-2,3- | | 2.34 (m, 9H), 2.22-2.13 (m, 1H), |
| | | dihydro-1H-1,3- | | 2.05 (d, J = 8.3 Hz, 2H), 2.00-1.86 |
| | | benzodiazol-1- | | (m, 7H), 1.85-1.73 (m, 2H), 1.72- |
| | | yl]piperidine-2,6-dione | | 1.62 (m, 2H), 1.60-1.44 (m, 4H), |
| | | | | 1.42-1.24 (m, 2H), 1.02 (t, J = 7.3 |
| | | | | Hz, 3H). |
| 90 | I-76 | 3-(5-[1-[4-([6-[2- | 940.50 | (400 MHz, CD₃OD) δ 9.26 (s, 1H), |
| | | (butylamino)-7-[trans-4- | | 8.43 (s, 1H), 8.06 (d, J = 8.9 Hz, |
| | | hydroxycyclohexyl]-7H- | | 1H), 7.97 (s, 1H), 7.71 (d, J = 8.9 |
| | | pyrrolo[2,3-d]pyrimidin- | | Hz, 1H), 7.05-6.97 (m, 2H), 6.94 |
| | | 5-yl]pyridazin-3- | | (d, J = 8.2 Hz, 1H), 5.32 (dd, J = |
| | | yl]methyl)piperazin-1- | | 12.6, 5.4 Hz, 1H), 4.75-4.40 (m, |
| | | yl]-3,6,9,12- | | 1H), 3.89 (s, 2H), 3.79-3.72 (m, |
| | | tetraoxapentadecan-15- | | 3H), 3.68-3.63 (m, 8H), 3.62-3.58 |
| | | yl]-3-methyl-2-oxo-2,3- | | (m, 2H), 3.54 (dd, J = 5.7, 3.0 Hz, |
| | | dihydro-1H-1,3- | | 2H), 3.51-3.40 (m, 4H), 3.38 (s, |
| | | benzodiazol-1- | | 3H), 3.25-3.10 (m, 5H), 2.97-2.87 |
| | | yl)piperidine-2,6-dione; | | (m, 1H), 2.80-2.71 (m, 8H), 2.19- |
| | | formic acid | | 2.01 (m, 8H), 1.86 (p, J = 6.6 Hz, |
| | | | | 2H), 1.68 (p, J = 7.2 Hz, 2H), 1.60- |
| | | | | 1.41 (m, 4H), 1.02 (t, J = 7.4 Hz, |
| | | | | 3H). |
| 91 | | 5-[(3aS,4S,6aR)-2-oxo- | 820.55 | (400 MHz, CDCl₃) δ 8.79 (s, 1H), |
| | | hexahydro-1H- | | 7.56 (d, J = 8.0 Hz, 2H), 7.41 (d, J = |
| | | thieno[3,4-d]imidazol-4- | | 7.7 Hz, 2H), 7.09 (s, 1H), 6.08 (s, |
| | | yl]-N-[2-(2-[2-[4-([4-[2- | | 1H), 5.27 (s, 1H), 4.74 (s, 1H), |
| | | (butylamino)-7-[trans-4- | | 4.59-4.48 (m, 2H), 4.38 (t, J = 6.3 |
| | | hydroxycyclohexyl]-7H- | | Hz, 1H), 3.95 (s, 2H), 3.71-3.55 (m, |
| | | pyrrolo[2,3-d]pyrimidin- | | 10H), 3.58-3.35 (m, 5H), 3.19-2.89 |
| | | 5- | | (m, 10H), 2.74 (d, J = 12.8 Hz, 1H), |
| | | yl]phenyl]methyl)piperazin- | | 2.40-2.28 (m, 2H), 2.24-2.20 (m, |
| | | 1- | | 4H), 1.93-1.53 (m, 11H), 1.51-1.42 |
| | | yl]ethoxy]ethoxy)ethyl] | | (m, 5H), 1.00 (t, J = 7.3 Hz, 3H). |
| | | pentanamide | | |
| 92 | I-88 | 3-(5-[3-[2-(2-[4-[(4-[2- | 854.25 | (400 MHz, CD₃OD) δ 8.70 (s, 1H), |
| | | [(3-fluoropropyl)amino]- | | 8.52 (s, 1H), 7.59 (d, J = 7.9 Hz, |
| | | 7-[trans-4-hydroxycyclo- | | 2H), 7.43-7.31 (m, 3H), 7.05-6.92 |
| | | hexyl]-7H-pyrrolo[2,3-d] | | (m, 3H), 5.30 (dd, J = 12.6, 5.4 Hz, |
| | | pyrimidin-5yl]phenyl) | | 1H), 4.55 (q, J = 6.4, 5.7 Hz, 2H), |
| | | methyl]piperazin-1- | | 3.74 (m, 2H), 3.69-3.56 (m, 8H), |
| | | yl]ethoxy)ethoxy)propyl]- | | 3.49 (t, J = 6.4 Hz, 2H), 3.39 (s, |
| | | 3-methyl-2-oxo-2,3- | | 3H), 3.04-2.86 (m, 8H), 2.82-2.65 |
| | | dihydro-1H-1,3- | | (m, 10H), 2.18-1.99 (m, 8H), 1.94- |
| | | benzodiazol-1- | | 1.85 (m, 2H), 1.54 (p, J = 10.6 Hz, |
| | | yl)piperidine-2,6-dione; | | 2H). |
| | | formic acid | | |
| 93 | I-83 | 3-[5-[3-(3-[4-[(4-[7- | 844.60 | (400 MHz, CD₃OD) δ 8.85 (s, 1H), |
| | | cyclohexyl-2-[(3,3,3- | | 7.91 (s, 1H), 7.69 (d, J = 8.1 Hz, |
| | | trifluoropropyl)amino]- | | 2H), 7.49 (d, J = 8.0 Hz, 2H), 7.05- |
| | | 7H-pyrrolo[2,3- | | 7.01 (m, 2H), 6.98 (d, J = 8.3 Hz, |
| | | d]pyrimidin-5- | | 1H), 5.33 (dd, J = 12.6, 5.5 Hz, |
| | | yl]phenyl)methyl]piperazin- | | 1H), 4.70-4.60 (m, 1H), 3.86 (t, J = |
| | | 1-yl]propoxy)propyl]- | | 7.0 Hz, 2H), 3.80 (s, 2H), 3.56-3.46 |
| | | 3-methyl-2-oxo-2,3- | | (m, 4H), 3.41 (s, 3H), 3.21-3.14 (m, |
| | | dihydro-1H-1,3- | | 2H), 2.99-2.88 (m, 2H), 2.86-2.75 |
| | | benzodiazol-1- | | (m, 6H), 2.73-2.61 (m, 3H), 2.22- |
| | | yl]piperidine-2,6-dione; | | 2.14 (m, 1H), 2.12-2.06 (m, 2H), |
| | | tris(trifluoroacetic acid) | | 2.05-1.88 (m, 9H), 1.83 (d, J = 12.9 |
| | | | | Hz, 1H), 1.63-1.50 (m, 2H), 1.48- |
| | | | | 1.35 (m, 1H). |
| 94 | I-92 | 3-(3-methyl-2-oxo-5-[3- | [M/2 + | (400 MHz, CD₃OD) δ 8.77 (s, 1H), |
| | | [2-(3-[4-[(4-[7-[trans-4- | 1]⁺ = | 8.43 (s, 1H), 7.64 (d, J = 8.0 Hz, |
| | | hydroxycyclohexyl]-2- | 449.40 | 2H), 7.47 (s, 1H), 7.41 (d, J = 8.0 |
| | | [(3,3,3- | | Hz, 2H), 7.29 (d, J = 1.5 Hz, 1H), |
| | | trifluoropropyl)amino]- | | 7.24 (dd, J = 8.2, 1.5 Hz, 1H), 7.11 |
| | | 7H-pyrrolo[2,3- | | (d, J = 8.2 Hz, 1H), 5.35 (dd, J = |
| | | d]pyrimidin-5- | | 12.4, 5.4 Hz, 1H), 4.55 (q, J = 8.4, |
| | | yl]phenyl)methyl]piperazin- | | 8.0 Hz, 1H), 4.46 (s, 2H), 4.18 (dd, |
| | | 1-yl]azetidin-1- | | J = 10.6, 7.1 Hz, 2H), 3.94 (dd, J = |
| | | yl)ethoxy]prop-1-yn-1- | | 10.6, 6.4 Hz, 2H), 3.87-3.80 (m, |
| | | yl]-2,3-dihydro-1H-1,3- | | 2H), 3.80-3.68 (m, 5H), 3.46 (s, |
| | | benzodiazol-1- | | 3H), 3.45-3.36 (m, 2H), 3.30 (d, J = |
| | | yl)piperidine-2,6-dione; | | 6.7 Hz, 2H), 2.98-2.74 (m, 4H), |
| | | formic acid | | 2.63-2.54 (m, 5H), 2.53-2.40 (m, |
| | | | | 3H), 2.21-2.03 (m, 7H), 1.55 (p, J = |
| | | | | 10.6 Hz, 2H). |
| 95 | I-91 | 3-(3-methyl-2-oxo-5-[3- | 901.40 | (400 MHz, CD₃OD) δ 8.76 (s, 1H), |
| | | [2-(3-[4-[(4-[7-[trans-4- | | 8.48 (s, 1H), 7.64 (d, J = 8.1 Hz, |
| | | hydroxycyclohexyl]-2- | | 2H), 7.46 (s, 1H), 7.41 (d, J = 8.0 |
| | | [(3,3,3- | | Hz, 2H), 7.08-7.00 (m, 2H), 6.98 |
| | | trifluoropropyl)amino]- | | (dd, J = 8.2, 1.5 Hz, 1H), 5.33 (dd, |
| | | 7H-pyrrolo[2,3- | | J = 12.5, 5.4 Hz, 1H), 4.61-4.52 (m, |
| | | d]pyrimidin-5- | | 1H), 4.14 (dd, J = 10.4, 7.1 Hz, |
| | | yl]phenyl)methyl]piperazin- | | 2H), 3.94-3.86 (m, 2H), 3.75-3.66 |
| | | 1-yl]azetidin-1- | | (m, 6H), 3.62 (t, J = 4.9 Hz, 2H), |
| | | yl)ethoxy]propyl]-2,3- | | 3.52 (t, J = 6.4 Hz, 2H), 3.43 (s, |
| | | dihydro-1H-1,3- | | 3H), 3.30 (d, J = 4.7 Hz, 2H), 2.95- |
| | | benzodiazol-1- | | 2.87 (m, 1H), 2.87-2.42 (m, 14H), |
| | | yl)piperidine-2,6-dione; | | 2.23-2.03 (m, 7H), 2.01-1.90 (m, |
| | | formic acid | | 2H), 1.55 (p, J = 11.1 Hz, 2H). |
| 96 | I-94 | 3-(3-methyl-2-oxo-5-[3- | 929.45 | (400 MHz, CD₃OD) δ 8.76 (s, 1H), |
| | | [2-(4-[4-[(4-[7-[trans-4- | | 8.53 (s, 1H), 7.63 (d, J = 8.0 Hz, |
| | | hydroxycyclohexyl]-2- | | 2H), 7.48-7.38 (m, 3H), 7.08-7.01 |
| | | [(3,3,3- | | (m, 2H), 7.01-6.95 (m, 1H), 5.33 |
| | | trifluoropropyl)amino]- | | (dd, J = 12.5, 5.4 Hz, 1H), 4.61 (m, |
| | | 7H-pyrrolo[2,3- | | 1H), 4.56 (d, J = 7.5 Hz, 1H), 3.79- |
| | | d]pyrimidin-5- | | 3.63 (m, 7H), 3.52 (t, J = 6.3 Hz, |
| | | yl]phenyl)methyl]piperazin- | | 2H), 3.43 (s, 3H), 3.37 (d, J = 4.6 |
| | | 1-yl]piperidin-1- | | Hz, 2H), 3.01-2.87 (m, 3H), 2.85- |
| | | yl)ethoxy)propyl]-2,3- | | 2.74 (m, 7H), 2.72-2.54 (m, 9H), |
| | | dihydro-1H-1,3- | | 2.16-2.02 (m, 9H), 1.96 (p, J = 6.7 |
| | | benzodiazol-1- | | Hz, 2H), 1.73 (d, J = 12.8 Hz, 2H), |
| | | yl)piperidine-2,6-dione; | | 1.60-1.50 (m, 2H). |
| | | formic acid | | |
| 97 | I-85 | 3-(3-methyl-2-oxo-5-[3- | 911.35 | (400 MHz, CD₃OD) δ 8.79 (d, J = |
| | | [2-(3-[4-[(4-[7-[trans-4- | | 1.3 Hz, 1H), 8.23 (s, 2H), 7.71 (d, J = |
| | | hydroxycyclohexyl]-2- | | 7.9 Hz, 2H), 7.54 (s, 1H), 7.50- |
| | | [(3,3,3- | | 7.43 (m, 2H), 7.31 (d, J = 1.4 Hz, |
| | | trifluoropropyl)amino]- | | 1H), 7.26 (dt, J = 8.2, 1.6 Hz, 1H), |
| | | 7H-pyrrolo[2,3- | | 7.13 (dd, J = 8.2, 2.2 Hz, 1H), 5.37 |
| | | d]pyrimidin-5- | | (dt, J = 12.4, 4.8 Hz, 1H), 4.56 (q, |
| | | yl]phenyl)methyl]piperazin- | | J = 7.7 Hz, 1H), 4.51 (s, 2H), 4.02 |
| | | 1-yl]pyrrolidin-1- | | (s, 2H), 3.94 (t, J = 4.9 Hz, 2H), |
| | | yl)ethoxy]prop-1-yn-1- | | 3.78-3.70 (m, 3H), 3.59 (dd, J = |
| | | yl]-2,3-dihydro-1H-1,3- | | 11.5, 7.1 Hz, 1H), 3.55-3.38 (m, |
| | | benzodiazol-1- | | 8H), 3.23-3.15 (m, 1H), 3.00-2.83 |
| | | yl)piperidine-2,6-dione; | | (m, 5H), 2.83-2.75 (m, 2H), 2.68- |
| | | di(formic acid) | | 2.52 (m, 5H), 2.32-2.22 (m, 1H), |
| | | | | 2.19-2.00 (m, 8H), 1.56 (q, J = |
| | | | | 10.2, 9.3 Hz, 2H). |
| 98 | I-93 | 3-(3-methyl-2-oxo-5-[3- | 915.45 | (400 MHz, CD₃OD) δ 8.77 (s, 1H), |
| | | [2-(3-[4-[(4-[7-[trans-4- | | 8.25 (s, 1H), 7.71-7.64 (m, 2H), |
| | | hydroxycyclohexyl]-2- | | 7.52-7.42 (m, 3H), 7.09-6.95 (m, |
| | | [(3,3,3- | | 3H), 5.38-5.32 (m, 1H), 4.70-4.54 |
| | | trifluoropropyl)amino]- | | (m, 2H), 3.88 (m, 2H), 3.79-3.68 |
| | | 7H-pyrrolo[2,3- | | (m, 6H), 3.57 (t, J = 6.4 Hz, 2H), |
| | | d]pyrimidin-5- | | 3.55-3.40 (m, 2H), 3.43 (s, 3H), |
| | | yl]phenyl)methyl]piperazin- | | 3.40-3.30 (m, 2H), 3.20 (t, J = 7.1 |
| | | 1-yl]pyrrolidin-1- | | Hz, 1H), 3.01-2.50 (m, 15H), 2.31- |
| | | yl)ethoxy)propyl]2,3- | | 2.22 (m, 1H), 2.22-1.93 (m, 10H), |
| | | dihydro-1H-1,3- | | 1.55 (p, J = 11.0 Hz, 2H). |
| | | benzodiazol-1- | | |
| | | yl)piperidine-2,6-dione; | | |
| | | formic acid | | |
| 99 | I-95 | 3-(3-methyl-2-oxo-5-[3- | 929.30 | (400 MHz, CD₃OD) δ 8.74 (d, J = |
| | | [2-(3-[4-[(4-[7-[trans-4- | | 1.3 Hz, 1H), 8.50 (s, 1H), 7.63-7.57 |
| | | hydroxycyclohexyl]-2- | | (m, 2H), 7.46 (s, 1H), 7.36 (dd, J = |
| | | [(3,3,3- | | 8.2, 2.1 Hz, 2H), 7.07-6.95 (m, |
| | | trifluoropropyl)amino]- | | 3H), 5.36-5.27 (m, 1H), 4.56 (p, J = |
| | | 7H-pyrrolo[2,3- | | 8.7 Hz, 1H), 3.80-3.61 (m, 7H), |
| | | d]pyrimidin-5- | | 3.54 (t, J = 6.4 Hz, 2H), 3.41 (s, |
| | | yl]phenyl)methyl]piperazin- | | 3H), 3.25-3.05 (m, 4H), 2.93 (td, J = |
| | | 1-yl]piperidin-1- | | 13.2, 6.5 Hz, 2H), 2.84-2.73 (m, |
| | | yl)ethoxy]propyl]2,3- | | 10H), 2.72-2.54 (m, 6H), 2.19-2.03 |
| | | dihydro-1H-1,3- | | (m, 7H), 2.03-1.92 (m, 4H), 1.76- |
| | | benzodiazol-1- | | 1.48 (m, 4H). |
| | | yl)piperidine-2,6-dione; | | |
| | | formic acid | | |
| 100 | I-86 | 3-(3-methyl-2-oxo-5-[3- | 925.40 | (400 MHz, DMSO-d₆) δ 11.14 (s, |
| | | [2-(4-[4-[(4-[7-[trans-4- | | 1H), 8.88 (s, 1H), 8.21 (s, 1H), |
| | | hydroxycyclohexyl]-2- | | 7.66-7.60 (m, 3H), 7.36-7.27 (m, |
| | | [(3,3,3- | | 3H), 7.20-7.11 (m, 2H), 7.07 (d, J = |
| | | trifluoropropyl)amino]- | | 6.1 Hz, 1H), 5.40 (dd, J = 12.8, |
| | | 7H-pyrrolo[2,3- | | 5.4 Hz, 1H), 4.71 (s, 1H), 4.48-4.40 |
| | | d]pyrimidin-5- | | (m, 1H), 4.38 (s, 2H), 3.62-3.50 (m, |
| | | yl]phenyl)methyl]piperazin- | | 6H), 3.44 (s, 2H), 3.40-3.35 (m, |
| | | 1-yl]piperidin-1- | | 7H), 2.96-2.82 (m, 3H), 2.75-2.52 |
| | | yl)ethoxy]prop-1-yn-1- | | (m, 5H), 2.45-2.30 (m, 3H), 2.15- |
| | | yl]-2,3-dihydro-1H-1,3- | | 2.08 (m, 1H), 2.05-1.88 (m, 9H), |
| | | benzodiazol-1-yl)piperidine- | | 1.70 (d, J = 11.2 Hz, 2H), 1.43-1.30 |
| | | 2,6-dione; formic acid | | (m, 4H). |
| 101 | I-87 | 3-(3-methyl-2-oxo-5-[3- | 925.35 | (400 MHz, CD₃OD) δ 8.78 (s, 1H), |
| | | [2-(3-[4-[(4-[7-[trans-4- | | 8.16 (s, 1H), 7.72 (d, J = 7.8 Hz, |
| | | hydroxycyclohexyl]-2- | | 2H), 7.56 (d, J = 1.3 Hz, 1H), 7.51- |
| | | [(3,3,3- | | 7.44 (m, 2H), 7.30 (d, J = 1.4 Hz, |
| | | trifluoropropyl)amino]- | | 1H), 7.25 (dt, J = 8.2, 1.8 Hz, 1H), |
| | | 7H-pyrrolo[2,3- | | 7.12 (dd, J = 8.2, 2.3 Hz, 1H), 5.36 |
| | | d]pyrimidin-5- | | (dt, J = 12.2, 4.8 Hz, 1H), 4.57 (t, J = |
| | | yl]phenyl)methyl]piperazin- | | 7.9 Hz, 1H), 4.51 (s, 2H), 4.13 (s, |
| | | 1-yl]piperidin-1- | | 2H), 3.98 (s, 2H), 3.80-3.70 (m, |
| | | yl)ethoxy]prop-1-yn-1- | | 3H), 3.52-3.45 (m, 7H), 3.30-3.00 |
| | | yl]-2,3-dihydro-1H-1,3- | | (m, 7H), 2.97-2.66 (m, 7H), 2.60 |
| | | benzodiazol-1- | | (dt, J = 11.3, 7.3 Hz, 2H), 2.26-1.88 |
| | | yl)piperidine-2,6-dione; | | (m, 9H), 1.87-1.62 (m, 2H), 1.55 |
| | | formic acid | | (p, J = 11.0 Hz, 2H). |
| 102 | I-154 | 3-[3-methyl-2-oxo-5-(3- | 896.50 | (400 MHz, CD₃OD) δ 8.46 (s, 1H), |
| | | [2-[2-(4-[[(1r,4r)-4-[7- | | 7.07 (s, 1H), 7.04-6.96 (m, 2H), |
| | | [trans-4- | | 6.84 (s, 1H), 5.31 (dd, J = 12.4, 5.5 |
| | | hydroxycyclohexyl]-2- | | Hz, 1H), 4.44 (p, J = 8.4 Hz, 1H), |
| | | [(3,3,3- | | 3.74-3.65 (m, 5H), 3.64-3.58 (m, |
| | | trifluoropropyl)amino] | | 4H), 3.50 (t, J = 6.3 Hz, 2H), 3.43 |
| | | pyrrolo[2,3-d]pyrimidin-5- | | (s, 3H), 2.97-2.85 (m, 1H), 2.85- |
| | | yl]cyclohexyl]methyl] | | 2.71 (m, 4H), 2.71-2.42 (m, 13H), |
| | | piperazin-1- | | 2.22 (d, J = 6.9 Hz, 2H), 2.18-2.08 |
| | | yl)ethoxy]ethoxy]propyl)- | | (m, 3H), 2.07-1.96 (m, 6H), 1.96- |
| | | 1,3-benzodiazol-1- | | 1.86 (m, 4H), 1.67-1.40 (m, 5H), |
| | | yl]piperidine-2,6-dione | | 1.10 (q, J = 12.6 Hz, 2H). |
| 103 | I-153 | 3-[3-methyl-2-oxo-5-(3- | 896.55 | (400 MHz, CD₃OD) δ 8.46 (s, 1H), |
| | | [2-[2-(4-[[(1s,4s)-4-[7- | | 7.08-7.06 (m, 1H), 7.01 (q, J = 8.2 |
| | | [trans-4- | | Hz, 2H), 6.90-6.82 (m, 1H), 5.32 |
| | | hydroxycyclohexyl]-2- | | (dd, J = 12.3, 5.4 Hz, 1H), 4.51- |
| | | [(3,3,3- | | 43.7 (m, 1H), 3.76-3.65 (m, 5H), |
| | | trifluoropropyl)amino] | | 3.61 (m, 4H), 3.50 (t, J = 6.3 Hz, |
| | | pyrrolo[2,3-d]pyrimidin-5- | | 2H), 3.44 (s, 3H), 2.99-2.83 (m, |
| | | yl]cyclohexyl]methyl] | | 1H), 2.83-2.72 (m, 3H), 2.71-2.40 |
| | | piperazin-1- | | (m, 14H), 2.35 (d, J = 7.1 Hz, 2H), |
| | | yl)ethoxy]ethoxy]propyl)- | | 2.21-2.07 (m, 3H), 2.05-1.84 (m, |
| | | 1,3-benzodiazol-1- | | 7H), 1.77 (m, 4H), 1.70-1.57 (m, |
| | | yl]piperidine-2,6-dione | | 4H), 1.51 (p, J = 11.1 Hz, 2H). |
| 104 | I-89 | 3-[3-methyl-2-oxo-5-[1- | 944.55 | (400 MHz, DMSO-d₆) δ 8.34 (s, |
| | | (4-[[(1s,4s)-4-[2- | | 3H), 6.95 (s, 1H), 6.86-6.76 (m, |
| | | (butylamino)-7-[trans-4- | | 3H), 5.20-5.10 (m, 1H), 4.30-4.22 |
| | | hydroxycyclohexyl]-7H- | | (m, 1H), 3.65-3.50 (m, 17H), 3.44- |
| | | pyrrolo[2,3-d]pyrimidin- | | 3.31 (m, 3H), 3.28-3.18 (m, 5H), |
| | | 5- | | 2.85-2.63 (m, 11H), 2.60-2.40 (m, |
| | | yl]cyclohexyl]methyl] | | 4H), 2.02-1.85 (m, 4H), 1.82-1.71 |
| | | piperazin-1-yl)-3,6,9,12- | | (m, 4H), 1.70-1.58 (m, 4H), 1.57- |
| | | tetraoxapentadecan-15- | | 1.41 (m, 6H), 1.39-1.10 (m, 6H), |
| | | yl]-2,3-dihydro-1H-1,3- | | 0.80 (t, J = 7.4 Hz, 3H). |
| | | benzodiazol-1- | | |
| | | yl]piperidine-2,6-dione; | | |
| | | di(formic acid) | | |
| 105 | I-90 | 3-[3-methyl-2-oxo-5-[1- | 944.70 | (400 MHz, DMSO-d₆) δ 8.26 (s, |
| | | (4-[[(1r,4r)-4-[2- | | 1H), 6.94 (s, 1H), 6.88 (s, 1H), 6.85 |
| | | (butylamino)-7-[trans-4- | | (s, 2H), 5.20 (dd, J = 13.1, 5.5 Hz, |
| | | hydroxycyclohexyl]-7H- | | 1H), 4.33-4.18 (m, 1H), 3.71-3.48 |
| | | pyrrolo[2,3-d]pyrimidin- | | (m, 16H), 3.42 (t, J = 6.5 Hz, 2H), |
| | | 5- | | 3.28 (m, 3H), 3.21 (s, 3H), 3.05- |
| | | yl]cyclohexyl]methyl] | | 2.36 (m, 16H), 2.06-1.89 (m, 3H), |
| | | piperazin-1-yl)-3,6,9,12- | | 1.84 (d, J = 12.0 Hz, 2H), 1.80-1.71 |
| | | tetraoxapentadecan-15- | | (m, 14H), 1.70-1.52 (m, 4H), 1.48 |
| | | yl]-2,3-dihydro-1H-1,3- | | (p, J = 7.1 Hz, 2H), 1.43-1.09 (m, |
| | | benzodiazol-1- | | 7H), 0.99 (d, J = 12.6 Hz, 2H), 0.81 |
| | | yl]piperidine-2,6-dione | | (t, J = 7.4 Hz, 3H). |
| 106 | I-60 | 3-[5-[1-(4-[4-[2- | 1002.50 | (400 MHz, CDCl₃) δ 8.71 (s, 1H), |
| | | (butylamino)-7-[trans-4- | | 8.28 (s, 1H), 7.82-7.76 (m, 2H), |
| | | hydroxycyclohexyl]-7H- | | 7.69 (d, J = 8.3 Hz, 2H), 7.26 (s, |
| | | pyrrolo[2,3-d]pyrimidin- | | 1H), 6.95-6.91 (m, 2H), 6.73 (d, J = |
| | | 5- | | 7.9 Hz, 1H), 6.56 (s, 1H), 5.23- |
| | | yl]benzenesulfonyl] | | 5.18 (m, 1H), 4.63-4.54 (m, 1H), |
| | | piperazin-1-yl)-3,6,9,12- | | 3.80 (dq, J = 10.8, 5.4, 4.2 Hz, 1H), |
| | | tetraoxapentadecan-15- | | 3.71-3.62 (m, 7H), 3.62-3.58 (m, |
| | | yl]-3-methyl-2-oxo-2,3- | | 6H), 3.55-3.46 (m, 4H), 3.44 (s, |
| | | dihydro-1H-1,3- | | 3H), 3.27 (s, 3H), 3.26-3.18 (m, |
| | | benzodiazol-1-yl]-1- | | 3H), 3.09-3.02 (m, 2H), 2.89-2.72 |
| | | methylpiperidine-2,6- | | (m, 11H), 2.23-2.13 (m, 5H), 2.00- |
| | | dion; formic acid | | 1.87 (m, 4H), 1.72-1.51 (m, 4H), |
| | | | | 1.53-1.45 (m, 2H), 1.01 (t, J = 7.3 |
| | | | | Hz, 3H). |
| 107 | I-79 | 3-[3-methyl-2-oxo-5-[3- | [(M/2 + | (400 MHz, CD₃OD) δ 8.76 (s, 1H), |
| | | (4-[4-[(4-[7-[trans-4- | 1)]⁺ = | 8.56 (s, 1H), 7.66-7.59 (m, 2H), |
| | | hydroxycyclohexyl]-2- | 438.05 | 7.46 (s, 1H), 7.44-7.38 (m, 2H), |
| | | [(3,3,3- | | 7.07-7.03 (m, 1H), 7.01 (s, 1H), |
| | | trifluoropropyl)amino]- | | 6.97 (dd, J = 8.1, 1.5 Hz, 1H), 5.33 |
| | | 7H-pyrrolo[2,3- | | (dd, J = 12.3, 5.4 Hz, 1H), 4.56 (p, |
| | | d]pyrimidin-5- | | J = 8.2 Hz, 1H), 3.77-3.69 (m, 3H), |
| | | yl]phenyl)methyl] | | 3.66 (s, 2H), 3.52-3.45 (m, 4H), |
| | | piperazin-1-yl]butoxy)propyl]- | | 3.43 (s, 3H), 2.98-2.53 (m, 17H), |
| | | 2,3-dihydro-1H-1,3- | | 2.22-2.04 (m, 7H), 1.97-1.86 (m, |
| | | benzodiazol-1- | | 2H), 1.75-1.49 (m, 6H). |
| | | yl]piperidine-2,6-dione; | | |
| | | formic acid | | |
| 108 | I-84 | 3-[5-[3-(4-[4-[(4-[7- | [(M/2 + | (400 MHz, CD₃OD) δ 8.75 (s, 1H), |
| | | cyclohexyl-2-[(3,3,3- | 1)]⁺ + | 7.64 (d, J = 7.9 Hz, 2H), 7.48-7.38 |
| | | trifluoropropyl)amino]- | 430.00 | (m, 3H), 7.06-6.94 (m, 3H), 5.32 |
| | | 7H-pyrrolo[2,3- | | (dd, J = 12.3, 5.4 Hz, 1H), 4.62- |
| | | d]pyrimidin-5- | | 4.50 (m, 1H), 3.75-3.63 (m, 4H), |
| | | yl]phenyl)methyl]piperazin- | | 3.51-3.44 (m, 4H), 3.43 (s, 3H), |
| | | 1-yl]butoxy)propyl]-3- | | 2.95-2.52 (m, 17H), 2.21-2.12 (m, |
| | | methyl-2-oxo-2,3- | | 1H), 2.06 (d, J = 11.4 Hz, 2H), |
| | | dihydro-1H-1,3- | | 2.02-1.88 (m, 6H), 1.81 (d, J = 12.5 |
| | | benzodiazol-1- | | Hz, 1H), 1.74-1.48 (m, 6H), 1.39 (t, |
| | | yl]piperidine-2,6-dione | | J = 12.9 Hz, 1H). |
| 109 | I-81 | 3-[3-methyl-2-oxo-5-[3- | [(M/2 + | (400 MHz, CD₃OD) δ 8.75 (s, 1H), |
| | | (4-[4-[1-(4-[7-[trans-4- | 1)]⁺ = | 7.66-7.59 (m, 2H), 7.44 (s, 1H), |
| | | hydroxycyclohexyl]-2- | 445.00 | 7.42-7.38 (m, 2H), 7.07-6.99 (m, |
| | | [(3,3,3- | | 2H), 6.96 (dd, J = 8.2, 1.6 Hz, 1H), |
| | | trifluoropropyl)amino]- | | 5.32 (dd, J = 12.4, 5.4 Hz, 1H), 4.56 |
| | | 7H-pyrrolo[2,3- | | (p, J = 8.1 Hz, 1H), 3.79-3.69 (m, |
| | | d]pyrimidin-5- | | 3H), 3.54 (d, J = 6.8 Hz, 1H), 3.49- |
| | | yl]phenyl)ethyl]piperazin- | | 3.43 (m, 4H), 3.42 (s, 3H), 3.00- |
| | | 1-yl]butoxy)propyl]-2,3- | | 2.87 (m, 2H), 2.86-2.70 (m, 8H), |
| | | dihydro-1H-1,3- | | 2.65-2.52 (m, 7H), 2.21-2.12 (m, |
| | | benzodiazol-1- | | 3H), 2.11-2.06 (m, 4H), 1.96-1.84 |
| | | yl]piperidine-2,6-dione | | (m, 2H), 1.69-1.50 (m, 6H), 1.47 |
| | | | | (d, J = 6.7 Hz, 3H). |
| 110 | I-80 | 3-[3-methyl-2-oxo-5-[3- | 874.45 | (400 MHz, CD₃OD) δ 8.76 (s, 1H), |
| | | (3-[4-[1-(4-[7-[trans-4- | | 7.61 (d, J = 8.2 Hz, 2H), 7.50-7.30 |
| | | hydroxycyclohexyl]-2- | | (m, 3H), 7.06-6.99 (m, 2H), 6.96 |
| | | [(3,3,3- | | (dd, J = 8.1, 1.5 Hz, 1H), 5.32 (dd, |
| | | trifluoropropyl)amino]- | | J = 12.4, 5.4 Hz, 1H), 4.63 (s, 1H), |
| | | 7H-pyrrolo[2,3- | | 4.55 (q, J = 8.5, 7.9 Hz, 1H), 3.79- |
| | | d]pyrimidin-5- | | 3.68 (m, 3H), 3.49-3.39 (m, 8H), |
| | | yl]phenyl)ethyl]piperazin- | | 2.93 (m, 1H), 2.85-2.71 (m, 4H), |
| | | 1-yl]propoxy)propyl]- | | 2.68-2.40 (m, 11H), 2.20-2.03 (m, |
| | | 2,3-dihydro-1H-1,3- | | 7H), 1.90 (p, J = 6.5 Hz, 2H), 1.77 |
| | | benzodiazol-1- | | (p, J = 6.2 Hz, 2H), 1.61-1.47 (m, |
| | | yl]piperidine-2,6-dione | | 2H), 1.44 (d, J = 6.7 Hz, 3H). |
| 111 | I-101 | 3-[3-methyl-2-oxo-5-[3- | 874.55 | (400 MHz, CD₃OD) δ 8.75 (s, 1H), |
| | | (3-[4-[(1R)-1-(4-[7- | | 7.62 (d, J = 8.0 Hz, 2H), 7.44 (s, |
| | | [trans-4- | | 1H), 7.40 (d, J = 8.0 Hz, 2H), 7.07- |
| | | hydroxycyclohexyl]-2- | | 6.95 (m, 3H), 5.32 (dd, J = 12.5, 5.5 |
| | | [(3,3,3- | | Hz, 1H), 4.57 (d, J = 7.8 Hz, 1H), |
| | | trifluoropropyl)amino]- | | 3.74 (m, 3H), 3.62-3.41 (m, 9H), |
| | | 7H-pyrrolo[2,3- | | 2.94 (d, J = 4.5 Hz, 1H), 2.90-2.71 |
| | | d]pyrimidin-5- | | (m, 8H), 2.68-2.53 (m, 7H), 2.20- |
| | | yl]phenyl)ethyl]piperazin- | | 2.04 (m, 7H), 1.94-1.78 (m, 4H), |
| | | 1-yl]propoxy)propyl]- | | 1.59-1.50 (m, 2H), 1.47 (d, J = 6.7 |
| | | 2,3-dihydro-1H-1,3- | | Hz, 3H). |
| | | benzodiazol-1- | | |
| | | yl]piperidine-2,6-dione | | |
| 112 | I-102 | 3-[3-methyl-2-oxo-5-[3- | 874.40 | (400 MHz, CD₃OD) δ 8.75 (s, 1H), |
| | | (3-[4-[(1S)-1-(4-[7- | | 8.49 (s, 1H), 7.64 (d, J = 8.0 Hz, |
| | | hydroxycyclohexyl]-2- | | 2H), 7.48-7.38 (m, 3H), 7.03-6.93 |
| | | [(3,3,3- | | (m, 3H), 5.32 (dd, J = 12.5, 5.5 Hz, |
| | | trifluoropropyl)amino]- | | 1H), 4.62 (s, 1H), 4.56 (t, J = 7.9 |
| | | 7H-pyrrolo[2,3- | | Hz, 1H), 3.77-3.70 (m, 3H), 3.70- |
| | | d]pyrimidin-5- | | 3.62 (m, 1H), 3.54-3.39 (m, 7H), |
| | | yl]phenyl)ethyl]piperazin- | | 3.15-2.88 (m, 7H), 2.84-2.71 (m, |
| | | 1-yl]propoxy)propyl]- | | 6H), 2.66-2.53 (m, 3H), 2.22-2.03 |
| | | 2,3-dihydro-1H-1,3- | | (m, 7H), 1.96-1.87 (m, 4H), 1.62- |
| | | benzodiazol-1- | | 1.49 (m, 5H). |
| | | yl]piperidine-2,6-dione; | | |
| | | formic acid | | |
| 113 | I-75 | 3-[5-(3-[3-[4-(1-[4-[2- | 834.35 | (400 MHz, CD₃OD) δ 8.70 (s, 1H), |
| | | (butylamino)-7-[trans-4- | | 8.53 (s, 1H), 7.61 (d, J = 7.9 Hz, |
| | | hydroxycyclohexyl]-7H- | | 2H), 7.39 (m, 3H), 7.06-6.99 (m, |
| | | pyrrolo[2,3-d]pyrimidin- | | 2H), 6.96 (d, J = 7.8 Hz, 1H), 5.32 |
| | | 5- | | (dd, J = 12.6, 5.3 Hz, 1H), 4.61- |
| | | yl]phenyl]ethyl)piperazin- | | 4.54 (m, 1H), 3.72 (m, 1H), 3.56- |
| | | 1-yl]propoxy]propyl)-3- | | 3.39 (m, 10H), 2.97-2.55 (m, 15H), |
| | | methyl-2-oxo-2,3- | | 2.18-2.01 (m, 7H), 1.92-1.76 (m, |
| | | dihydro-1H-1,3- | | 4H), 1.68 (p, J = 7.4 Hz, 2H), 1.59- |
| | | benzodiazol-1- | | 1.40 (m, 7H), 1.02 (t, J = 7.4 Hz, |
| | | yl]piperidine-2,6-dione; | | 3H). |
| | | formic acid | | |
| 114 | I-103 | 3-[5-[3-(3-[4-[(1R)-1-[4- | 834.40 | (400 MHz, CD₃OD) δ 8.70 (s, 1H), |
| | | [2-(butylamino)-7-[trans- | | 7.62 (d, J = 8.2 Hz, 2H), 7.44-7.36 |
| | | 4-hydroxycyclohexyl]- | | (m, 3H), 7.08-6.91 (m, 3H), 5.32 |
| | | 7H-pyrrolo[2,3- | | (dd, J = 12.5, 5.5 Hz, 1H), 4.56 (p, |
| | | d]pyrimidin-5- | | J = 8.3 Hz, 1H), 3.74 (td, J = 10.8, |
| | | yl]phenyl]ethyl]piperazin- | | 5.3 Hz, 1H), 3.56 (d, J = 6.7 Hz, |
| | | 1-yl]propoxy)propyl]-3- | | 1H), 3.52-3.40 (m, 9H), 3.00-2.87 |
| | | methyl-2-oxo-2,3- | | (m, 2H), 2.87-2.57 (m, 13H), 2.21- |
| | | dihydro-1H-1,3- | | 2.01 (m, 7H), 1.96-1.78 (m, 4H), |
| | | benzodiazol-1- | | 1.68 (ddd, J = 12.4, 8.3, 6.5 Hz, |
| | | yl]piperidine-2,6-dione | | 2H), 1.60-1.42 (m, 7H), 1.02 (t, J = |
| | | | | 7.4 Hz, 3H). |
| 115 | I-104 | 3-[5-[3-(3-[4-[(1S)-1-[4- | 834.50 | (400 MHz, CD₃OD) δ 8.70 (s, 1H), |
| | | [2-(butylamino)-7-[trans- | | 7.61 (d, J = 8.1 Hz, 2H), 7.43-7.36 |
| | | 4-hydroxycyclohexyl]- | | (m, 3H), 7.06-6.99 (m, 2H), 6.96 |
| | | 7H-pyrrolo[2,3- | | (d, J = 8.0 Hz, 1H), 5.32 (dd, J = |
| | | d]pyrimidin-5- | | 12.5, 5.4 Hz, 1H), 4.56 (p, J = 8.0 |
| | | yl]phenyl]ethyl]piperazin- | | Hz, 1H), 3.78-3.68 (m, 1H), 3.56- |
| | | 1-yl]propoxy)propyl]-3- | | 3.48 (m, 1H), 3.48-3.38 (m, 9H), |
| | | methyl-2-oxo-2,3- | | 3.03-2.48 (m, 15H), 2.14-2.02 (m, |
| | | dihydro-1H-1,3- | | 7H), 1.90 (p, J = 6.8 Hz, 2H), 1.82 |
| | | benzodiazol-1- | | (t, J = 7.6 Hz, 2H), 1.68 (p, J = 7.1 |
| | | yl]piperidine-2,6-dione | | Hz, 2H), 1.59-1.43 (m, 7H), 1.02 (t, |
| | | | | J = 7.4 Hz, 3H). |
| 116 | I-105 | 1-methyl-3-(3-methyl-2- | 904.70 | (400 MHz, CD₃OD) δ 8.73 (s, 1H), |
| | | oxo-5-[3-[2-(2-[4-[(4-[7- | | 7.59 (d, J = 7.9 Hz, 2H), 7.44 (s, |
| | | [trans-4- | | 1H), 7.37 (d, J = 8.0 Hz, 2H), 7.10- |
| | | hydroxycyclohexyl]-2- | | 6.87 (m, 3H), 5.31 (dd, J = 13.0, 5.4 |
| | | [(3,3,3- | | Hz, 1H), 4.61-4.52 (m, 1H), 3.77- |
| | | trifluoropropyl)amino]- | | 3.69 (m, 5H), 3.68-3.58 (m, 6H), |
| | | 7H-pyrrolo[2,3- | | 3.50 (t, J = 6.3 Hz, 2H), 3.40 (s, |
| | | d]pyrimidin-5- | | 3H), 3.17 (s, 3H), 3.00-2.55 (m, |
| | | yl]phenyl)methyl]piperazin- | | 17H), 2.19-2.01 (m, 7H), 1.95-1.87 |
| | | 1- | | (m, 2H), 1.59-1.50 (m, 2H). |
| | | yl]ethoxy)ethoxy]propyl]- | | |
| | | 2,3-dihydro-1H-1,3- | | |
| | | benzodiazol-1- | | |
| | | yl)piperidine-2,6-dione | | |
| 117 | I-128 | 3-(3-methyl-2-oxo-5-[2- | 887.65 | (400 MHz, DMSO-d₆) δ 8.88 (d, J = |
| | | [3-(2-[4-[(4-[7-[trans-4- | | 2.4 Hz, 1H), 7.73-7.61 (m, 3H), |
| | | hydroxycyclohexyl]-2- | | 7.34 (d, J = 7.9 Hz, 2H), 7.08-6.96 |
| | | [(3,3,3- | | (m, 3H), 6.88 (d, J = 8.1 Hz, 1H), |
| | | trifluoropropyl)amino] | | 5.35-5.25 (m, 1H), 4.44 (d, J = 11.4 |
| | | pyrrolo[2,3-d]pyrimidin-5- | | Hz, 1H), 4.10-3.89 (m, 3H), 3.85- |
| | | yl]phenyl)methyl]piperazin- | | 3.68 (m, 4H), 3.63-3.50 (m, 6H), |
| | | 1-yl]ethoxy)azetidin- | | 3.25-3.03 (m, 2H), 2.93-2.51 (m, |
| | | 1-yl]ethyl]-1,3- | | 15H), 2.03-1.85 (m, 9H), 1.73-1.66 |
| | | benzodiazol-1- | | (m, 1H), 1.42-1.30 (m, 3H). |
| | | yl)piperidine-2,6-dione | | |
| 118 | I-124 | 3-(3-methyl-2-oxo-5-[2- | 901.65 | (400 MHz, CD₃OD) δ 8.76 (s, 1H), |
| | | [3-(2-[4-[(4-[7-[trans-4- | | 8.38 (s, 3H), 7.66 (d, J = 7.8 Hz, |
| | | hydroxycyclohexyl]-2- | | 2H), 7.50-7.40 (m, 3H), 7.16 (s, |
| | | [(3,3,3- | | 1H), 7.08 (q, J = 8.0 Hz, 2H), 5.35 |
| | | trifluoropropyl)amino]- | | (dd, J = 12.3, 5.3 Hz, 1H), 4.63- |
| | | 7H-pyrrolo[2,3- | | 4.49 (m, 1H), 4.31 (s, 1H), 3.82 (s, |
| | | d]pyrimidin-5- | | 2H), 3.77-3.65 (m, 6H), 3.58 (d, J = |
| | | yl]phenyl)methyl]piperazin- | | 11.0 Hz, 2H), 3.46-3.39 (m, 5H), |
| | | 1- | | 3.26 (s, 1H), 3.15-3.05 (m, 2H), |
| | | yl]ethoxy)pyrrolidin-1- | | 3.00-2.85 (m, 13H), 2.67-2.63 (m, |
| | | yl]ethyl]-2,3-dihydro- | | 2H), 2.35-2.02 (m, 9H), 1.54 (p, J = |
| | | 1H-1,3-benzodiazol-1- | | 10.7 Hz, 2H). |
| | | yl)piperidine-2,6-dione; | | |
| | | tris(formic acid) | | |
| 119 | I-112 | 3-(3-methyl-2-oxo-5-[3- | 885.55 | (400 MHz, CD₃OD) δ 8.84 (s, 1H), |
| | | [3-(2-[4-[(4-[7-[trans-4- | | 7.88 (s, 1H), 7.68 (d, J = 7.9 Hz, |
| | | hydroxycyclohexyl]-2- | | 2H), 7.49 (d, J = 8.0 Hz, 2H), 7.15- |
| | | [(3,3,3- | | 6.98 (m, 3H), 5.34 (dd, J = 12.4, 5.3 |
| | | trifluoropropyl)amino]- | | Hz, 1H), 4.70-4.60 (m, 1H), 4.00 |
| | | 7H-pyrrolo[2,3- | | (d, J = 11.2 Hz, 1H), 3.87 (t, J = 6.9 |
| | | d]pyrimidin-5- | | Hz, 2H), 3.80-3.70 (m, 5H), 3.65- |
| | | yl]phenyl)methyl]piperazin- | | 3.54 (m, 4H), 3.44 (s, 3H), 3.43- |
| | | 1-yl]ethyl)azetidin-1- | | 3.35 (m, 1H), 3.23 (d, J = 7.0 Hz, |
| | | yl]propyl]-2,3-dihydro- | | 2H), 3.13-3.03 (m, 2H), 3.00-2.76 |
| | | 1H-1,3-benzodiazol-1- | | (m, 10H), 2.67 (dt, J = 11.0, 7.2 Hz, |
| | | yl)piperidine-2,6-dione | | 2H), 2.47 (d, J = 6.6 Hz, 1H), 2.24- |
| | | | | 1.99 (m, 10H), 1.62-1.52 (m, 2H). |
| 120 | I-109 | 3-(3-methyl-2-oxo-5-[[3- | 873.45 | (400 MHz, DMSO-d₆) δ 11.11 (s, |
| | | (2-[4-[(4-[7-[trans-4- | | 1H), 8.89 (s, 1H), 7.77-7.59 (m, |
| | | hydroxycyclohexyl]-2- | | 3H), 7.33 (d, J = 7.9 Hz, 2H), 7.17 |
| | | [3,3,3- | | (s, 1H), 7.13-6.93 (m, 3H), 5.37 |
| | | trifluoropropyl)amino]- | | (dd, J = 12.7, 5.5 Hz, 1H), 4.72 (d, |
| | | 7H-pyrrolo[2,3- | | J = 4.4 Hz, 1H), 4.41 (d, J = 12.9 |
| | | d]pyrimidin-5- | | Hz, 1H), 4.10-3.88 (m, 3H), 3.74 |
| | | yl]phenyl)methyl]piperazin- | | (m, 5H), 3.65-3.48 (m, 7H), 3.16 |
| | | 1-yl]ethoxy)azetidin- | | (d, J = 10.4 Hz, 2H), 2.98-2.85 (m, |
| | | 1-yl]methyl]2,3- | | 1H), 2.80-2.54 (m, 11H), 2.22 (s, |
| | | dihydro-1H-1,3- | | 2H), 2.07-1.83 (m, 8H), 1.57 (s, |
| | | benzodiazol-1- | | 1H), 1.45-1.26 (m, 2H). |
| | | yl)piperidine-2,6-dione | | |
| 121 | I-98 | 3-(3-methyl-2-oxo-5-[[3- | 887.40 | (400 MHz, CD₃OD) δ 8.76 (s, 1H), |
| | | (2-[4-[(4-[7-[trans-4- | | 8.47 (s, 1H), 7.64 (d, J = 7.8 Hz, |
| | | hydroxycyclohexyl]-2- | | 2H), 7.46 (s, 1H), 7.42 (dd, J = 8.3, |
| | | [(3,3,3- | | 2.2 Hz, 2H), 7.31 (s, 1H), 7.26-7.14 |
| | | trifluoropropyl)amino]- | | (m, 2H), 5.43-5.35 (m, 1H), 4.56 |
| | | 7H-pyrrolo[2,3- | | (q, J = 7.8 Hz, 1H), 4.32-4.11 (m, |
| | | d]pyrimidin-5- | | 3H), 3.79-3.58 (m, 7H), 3.47 (s, |
| | | yl]phenyl)methyl]piperazin- | | 3H), 3.28-3.20 (m, 1H), 3.15-3.07 |
| | | 1- | | (m, 2H), 3.03-2.75 (m, 10H), 2.75- |
| | | yl]ethoxy)pyrrolidin-1- | | 2.52 (m, 6H), 2.34-2.23 (m, 1H), |
| | | yl]methyl-2,3-dihydro- | | 2.22-2.03 (m, 8H), 1.62-1.50 (m, |
| | | 1H-1,3-benzodiazol-1- | | 2H). |
| | | yl)piperidine-2,6-dione; | | |
| | | formic acid | | |
| 122 | I-96 | 3-[5-(1-[4-[2- | 845.45 | (400 MHz, DMSO-d₆) δ 8.43 (s, |
| | | (butylamino)-7-[trans-4- | | 1H), 8.35 (s, 1H), 7.09 (s, 1H), 6.79 |
| | | hydroxycyclohexyl]-7H- | | (d, J = 8.1 Hz, 1H), 6.67 (s, 1H), |
| | | pyrrolo[2,3-d]pyrimidin- | | 6.60 (d, J = 8.1 Hz, 1H), 5.87 (s, |
| | | 5-yl]-1,2,3,6- | | 1H), 5.22 (dd, J = 13.0, 5.5 Hz, |
| | | tetrahydropyridin-1-yl]- | | 1H), 4.18-4.09 (m, 1H), 3.87-3.80 |
| | | 3,6,9,12- | | (m, 3H), 3.68-3.53 (m, 10H), 3.52- |
| | | tetraoxapentadecan-15- | | 3.40 (m, 4H), 3.39-3.30 (m, 4H), |
| | | yl)-3-methyl-2-oxo-2,3- | | 3.21 (s, 3H), 3.16-3.10 (m, 4H), |
| | | dihydro-1H-1,3- | | 2.88-2.77 (m, 2H), 2.69 (s, 2H), |
| | | benzodiazol-1- | | 2.57 (d, J = 13.1 Hz, 1H), 2.20 (t, J = |
| | | yl]piperidine-2,6-dione; | | 7.7 Hz, 2H), 2.10 (s, 1H), 1.91 (d, |
| | | formic acid | | J = 12.6 Hz, 2H), 1.77-1.52 (m, |
| | | | | 4H), 1.45 (d, J = 7.5 Hz, 2H), 1.41- |
| | | | | 1.28 (m, 4H), 1.24-1.09 (m, 2H) |
| | | | | 0.75 (t, J = 7.3 Hz, 3H). |
| 123 | I-97 | 3-[5-(1-[4-[2- | 847.45 | (400 MHz, CD₃OD) δ 8.51 (s, 1H), |
| | | (butylamino)-7-[trans-4- | | 8.31 (s, 2H), 7.05-6.99 (m, 2H), |
| | | hydroxycyclohexyl]-7H- | | 6.97-6.91 (m, 2H), 5.33 (dd, J = |
| | | pyrrolo[2,3-d]pyrimidin | | 12.6, 5.4 Hz, 1H), 4.45 (p, J = 8.0 |
| | | 5-yl]piperidin-1-yl]- | | Hz, 1H), 3.88-3.79 (m, 2H), 3.73- |
| | | 3,6,9,12- | | 3.61 (m, 13H), 3.56 (dd, J = 5.9 3.3 |
| | | tetraoxapentadecan-15- | | Hz, 2H), 3.45-3.39 (m, 7H), 3.16 (t, |
| | | yl)-3-methyl-2-oxo-2,3- | | J = 12.5 Hz, 2H), 3.09-2.97 (m, |
| | | dihydro-1H-1,3- | | 1H), 2.93 (dd, J = 13.8, 5.1 Hz, |
| | | benzodiazol-1- | | 1H), 2.86-2.77 (m, 2H), 2.72 (t, J = |
| | | yl]piperidine-2,6-dione; | | 7.5 Hz, 2H), 2.27-2.14 (m, 3H), |
| | | bis(formic acid) | | 2.13-2.02 (m, 5H), 2.01-1.93 (m, |
| | | | | 5H), 1.86 (p, J = 6.7 Hz, 2H), 1.64 |
| | | | | (p, J = 7.2 Hz, 2H), 1.58-1.40 (m, |
| | | | | 4H), 1.00 (t, J = 7.3 Hz, 3H). |
| 124 | I-111 | 3-[3-methyl-2-oxo-5-[3- | 874.30 | (400 MHz, CD₃OD) δ 8.83 (s, 1H), |
| | | (3-[4-[(4-[7-[trans-4- | | 7.91 (s, 1H), 7.69 (d, J = 8.0 Hz, |
| | | hydroxycyclohexyl]-2-[[(2R)- | | 2H), 7.51 (d, J = 7.7 Hz, 2H), 7.08- |
| | | 4,4,4-trifluorobutan-2- | | 6.96 (m, 3H), 5.33 (dd, J = 12.5, 5.4 |
| | | yl]amino]-7H-pyrrolo[2,3- | | Hz, 1H), 4.65 (dt, J = 12.9, 6.5 Hz, |
| | | d]pyrimidin-5-yl]phenyl) | | 2H), 4.10-3.90 (m, 2H), 3.80-3.70 |
| | | methyl]piperazin-1-yl] | | (m, 1H), 3.55-3.45 (m, 4H), 3.44- |
| | | propoxy)propyl]-2,3- | | 3.38 (m, 6H), 3.29-3.00 (m, 6H), |
| | | dihydro-1H-1,3-benzodiazol-1- | | 2.96-2.88 (m, 1H), 2.86-2.63 (m, |
| | | yl]piperidine-2,6-dione | | 5H), 2.62-2.48 (m, 1H), 2.22-2.04 |
| | | | | (m, 8H), 2.03-1.90 (m, 4H), 1.62- |
| | | | | 1.46 (m, 5H). |
| 125 | I-110 | 3-[3-methyl-2-oxo-5-[3- | 874.30 | (400 MHz, CD₃OD) δ 8.85 (d, J = |
| | | (3-[4-[(4-[7-[(1r,4r)-4- | | 6.1 Hz, 1H), 7.93 (s, 1H), 7.72 (d, J = |
| | | hydroxycyclohexyl]-2- | | 7.5 Hz, 2H), 7.54 (d, J = 7.2 Hz, |
| | | [[(2S)-4,4,4- | | 2H), 7.06-6.95 (m, 3H), 5.33 (dd, J = |
| | | trifluorobutan-2- | | 12.5, 5.4 Hz, 1H), 4.70-4.58 (m, |
| | | yl]amino]-7H- | | 2H), 4.04 (s, 2H), 3.74 (td, J = 10.9, |
| | | pyrrolo[2,3-d]pyrimidin- | | 5.3 Hz, 1H), 3.54 (t, J = 5.6 Hz, |
| | | 5- | | 2H), 3.49 (t, J = 6.2 Hz, 2H), 3.41 |
| | | yl]phenyl)methyl]piperazin- | | (s, 6H), 3.25-3.18 (m, 3H), 2.99- |
| | | 1-yl]propoxy) | | 2.87 (m, 4H), 2.87-2.64 (m, 6H), |
| | | propyl]2,3- | | 2.55 (ddd, J = 16.0, 10.8, 5.9 Hz, |
| | | dihydro-1H-1,3- | | 1H), 2.22-2.15 (m, 7H), 2.04-1.90 |
| | | benzodiazol-1- | | (m, 4H), 1.63-1.43 (m, 5H). |
| | | yl]piperidine-2,6-dione | | |
| 126 | I-106 | 3-[3-methyl-5-(3-[3- | 874.35 | (400 MHz, CD₃OD) δ 8.76 (s, 1H), |
| | | [(2S)-2-methyl-4-[(4-[7- | | 8.36 (s, 1H), 7.64 (d, J = 7.8 Hz, |
| | | [trans-4- | | 2H), 7.49-7.38 (m, 3H), 7.09-6.92 |
| | | hydroxycyclohexyl]-2- | | (m, 3H), 5.32 (dd, J = 12.4, 5.4 Hz, |
| | | [(3,3,3- | | 1H), 4.56 (p, J = 8.1 Hz, 1H), 3.78- |
| | | trifluoropropyl)amino]- | | 3.63 (m, 5H), 3.56-3.43 (m, 5H), |
| | | 7H-pyrrolo[2,3- | | 3.42 (s, 3H), 3.11-2.87 (m, 6H), |
| | | d]pyrimidin-5- | | 2.86-2.72 (m, 5H), 2.68-2.40 (m, |
| | | yl]phenyl)methyl]piperazin- | | 4H), 2.21-1.87 (m, 11H), 1.61-1.49 |
| | | 1-yl]propoxy]propyl)- | | (m, 2H), 1.35 (d, J = 6.4 Hz, 3H). |
| | | 2-oxo-2,3-dihydro-1H- | | |
| | | 1,3-benzodiazol-1- | | |
| | | yl]piperidine-2,6-dione; | | |
| | | formic acid | | |
| 127 | I-107 | 3-[3-methyl-5-(3-[3- | 874.35 | (400 MHz, CD₃OD) δ 8.76 (s, 1H), |
| | | [(2R)-2-methyl-4-[(4-[7- | | 8.32 (s, 2H), 7.64 (d, J = 8.1 Hz, |
| | | [trans-4- | | 2H), 7.47 (s, 1H), 7.42 (d, J = 7.9 |
| | | hydroxycyclohexyl]-2- | | Hz, 2H), 7.07-6.99 (m, 2H), 6.97 |
| | | [(3,3,3- | | (dd, J = 8.2, 1.5 Hz, 1H), 5.33 (dd, |
| | | trifluoropropyl)amino]- | | J = 12.5, 5.4 Hz, 1H), 4.46 (p, J = |
| | | 7H-pyrrolo[2,3- | | 8.3 Hz, 1H), 3.78-3.65 (m, 5H), |
| | | d]pyrimidin-5- | | 3.67-3.43 (m, 7H), 3.42 (s, 3H), |
| | | yl]phenyl)methyl]piperazin- | | 3.16-2.88 (m, 5H), 2.87-2.74 (m, |
| | | 1-yl]propoxy]propyl)- | | 4H), 2.68-2.40 (m, 4H), 2.17 (d, J = |
| | | 2-oxo-2,3-dihydro-1H- | | 6.7 Hz, 2H), 2.12-2.05 (m, 5H), |
| | | 1,3-benzodiazol-1- | | 2.02-1.88 (m, 4H), 1.55 (p, J = |
| | | yl]piperidine-2,6-dione; | | 11.7, 11.1 Hz, 2H), 1.35 (d, J = 6.4 |
| | | bis(formic acid) | | Hz, 3H). |
| 128 | I-100 | 3-[5-[3-(3-[4-[(4-[2- | 828.50 | (400 MHz, CD₃OD) δ 8.78 (s, 1H), |
| | | [(2,2- | | 8.49 (s, 1H), 7.68-7.61 (m, 2H), |
| | | difluoroethyl)amino]-7- | | 7.48 (s, 1H), 7.42 (d, J = 8.1 Hz, |
| | | [trans-4- | | 2H), 7.07-6.99 (m, 2H), 6.97 (dd, J = |
| | | hydroxycyclohexyl]-7H- | | 8.2, 1.5 Hz, 1H), 6.09 (tt, J = |
| | | pyrrolo[2,3-d]pyrimidin- | | 56.8, 4.2 Hz, 1H), 5.32 (dd, J = |
| | | 5- | | 12.4, 5.4 Hz, 1H), 4.57 (p, J = 8.1 |
| | | yl]phenyl)methyl]piperazin- | | Hz, 1H), 3.84 (td, J = 14.5, 4.2 Hz, |
| | | 1-yl]propoxy)propyl]- | | 2H), 3.79-3.69 (m, 3H), 3.55-3.44 |
| | | 3-methyl-2-oxo-2,3- | | (m, 4H), 3.42 (s, 3H), 3.04-2.88 (m, |
| | | dihydro-1H-1,3- | | 7H), 2.87-2.73 (m, 8H), 2.22-2.10 |
| | | benzodiazol-1- | | (m, 3H), 2.09-2.03 (m, 4H), 1.96- |
| | | yl]piperidine-2,6-dione; | | 1.86 (m, 4H), 1.55 (p, J = 10.8 Hz, |
| | | formic acid | | 2H). |
| 129 | I-108 | 3-[5-(3-[3-[4-([4-[2- | 804.35 | (400 MHz, CD₃OD) δ 8.75 (s, 1H), |
| | | (cyclopropylamino)-7- | | 8.34 (s, 2H), 7.65 (d, J = 7.9 Hz, |
| | | [trans-4- | | 2H), 7.49 (s, 1H), 7.43 (d, J = 8.0 |
| | | hydroxycyclohexyl]-7H- | | Hz, 2H), 7.07-7.00 (m, 2H), 6.97 |
| | | pyrrolo[2,3-d]pyrimidin- | | (dd, J = 8.3, 1.5 Hz, 1H), 5.33 (dd, |
| | | 5- | | J = 12.4, 5.4 Hz, 1H), 4.59 (p, J = |
| | | yl]phenyl]methyl]piperazin- | | 8.0 Hz, 1H), 3.76 (s, 2H), 3.74-3.70 |
| | | 1-yl]propoxy]propyl)- | | (m, 1H), 3.55-3.45 (m, 4H), 3.42 (s, |
| | | 3-methyl-2-oxo-2,3- | | 3H), 3.18 (s, 3H), 3.07 (t, J = 7.7 |
| | | dihydro-1H-1,3- | | Hz, 2H), 2.97-2.84 (m, 3H), 2.82 (s, |
| | | benzodiazol-1- | | 2H), 2.81-2.73 (m, 5H), 2.20-2.03 |
| | | yl]piperidine-2,6-dione; | | (m, 7H), 2.01-1.91 (m, 4H), 1.54 |
| | | bis(formic acid) | | (p, J = 10.9 Hz, 2H), 0.83 (td, J = |
| | | | | 6.9, 4.8 Hz, 2H), 0.62-0.54 (m, 2H) |
| 130 | I-146 | 3-[3-methyl-2-oxo-5-[2- | 832.45 | (400 MHz, CD₃OD) δ 8.81 (s, 1H), |
| | | (2-[4-[(3-[7-[trans-4- | | 7.63 (s, 1H), 7.57 (d, J = 7.9 Hz, |
| | | hydroxycyclohexyl]-2- | | 1H), 7.47 (s, 1H), 7.39 (t, J = 7.6 |
| | | [(3,3,3- | | Hz, 1H), 7.21 (d, J = 7.4 Hz, 1H), |
| | | trifluoropropyl)amino] | | 7.09 (s, 1H), 7.01 (s, 2H), 5.33-5.26 |
| | | pyrrolo[2,3-d]pyrimidin-5- | | (m, 1H), 4.57 (s, 1H), 3.78-3.65 (m, |
| | | yl]phenyl)methyl]piperazin- | | 5H), 3.59 (d, J = 4.4 Hz, 4H), 3.42 |
| | | 1-yl]ethoxy)ethyl]- | | (s, 3H), 2.95-2.92 (m, 3H), 2.76 (d, |
| | | 1,3-benzodiazol-1- | | J = 14.5 Hz, 2H), 2.61-2.55 (m, |
| | | yl]piperidine-2,6-dione | | 5H), 2.54-2.46 (m, 6H), 2.14-2.03 |
| | | | | (m, 7H), 1.56-1.54 (m, 2H), 1.31 (s, |
| | | | | 1H) |
| 131 | I-147 | 3-[3-methyl-2-oxo-5-(2- | 756.40 | (400 MHz, CD₃OD) δ 8.59 (s, 1H), |
| | | [2-[4-([7-[(1r,4r)-4- | | 7.13-6.96 (m, 4H), 5.33 (dd, J = |
| | | hydroxycyclohexyl]-2- | | 12.5, 5.4 Hz, 1H), 4.47 (tt, J = 10.6, |
| | | [(3,3,3- | | 5.7 Hz, 1H), 3.73-3.68 (m, 5H), |
| | | trifluoropropyl)amino] | | 3.62 (s, 2H), 3.58 (t, J = 5.3 Hz, |
| | | pyrrolo[2,3-d]pyrimidin-5- | | 2H), 3.42 (s, 3H), 2.99-2.87 (m, |
| | | yl]methyl)piperazin-1- | | 3H), 2.86-2.72 (m, 2H), 2.63-2.40 |
| | | yl]ethoxy]ethyl)-1,3- | | (m, 11H), 2.19-1.90 (m, 8H), 1.52 |
| | | benzodiazol-1- | | (dd, J = 11.7, 5.5 Hz, 2H) |
| | | yl]piperidine-2,6-dione | | |
| 132 | I-115 | 3-[5-[3-(3-[4-[(4-[2- | 842.65 | (400 MHz, CD₃OD) δ 8.74 (s, 1H), |
| | | [(3,3- | | 8.52 (s, 1H), 7.64 (dd, J = 8.2, 2.1 |
| | | difluoropropyl)amino]-7- | | Hz, 2H), 7.46-7.37 (m, 3H), 7.07- |
| | | [(1r,4r)-4- | | 6.94 (m, 3H), 6.07 (tt, J = 56.7, 4.5 |
| | | hydroxycyclohexyl]-7H- | | Hz, 1H), 5.32 (dd, J = 12.6, 5.3 Hz, |
| | | pyrrolo[2,3-d]pyrimidin- | | 1H), 4.62-4.50 (m, 1H), 3.79-3.68 |
| | | 5- | | (m, 3H), 3.67-3.59 (m, 2H), 3.51- |
| | | yl]phenyl)methyl]piperazin- | | 3.48 (m, 4H), 3.42 (d, J = 2.8 Hz, |
| | | 1-yl]propoxy)propyl]- | | 3H), 3.14-3.69 (m, 15H), 2.31-2.10 |
| | | 3-methyl-2-oxo-2,3- | | (m, 5H), 2.08-2.04 (m, 4H), 1.94- |
| | | dihydro-1H-1,3- | | 1.91 (m, 4H), 1.57-1.55 (m, 2H) |
| | | benzodiazol-1- | | |
| | | yl]piperidine-2,6-dione; | | |
| | | formic acid | | |
| 133 | I-134 | 3-(5-[3-[2-(2-[4-[(4-[7- | 910.30 | (400 MHz, CD₃OD) δ 8.74 (s, 1H), |
| | | [trans-4- | | 8.60 (dd, J = 7.9, 1.5 Hz, 1H), 8.38 |
| | | hydroxycyclohexyl]-2- | | (dd, J = 4.9, 1.5 Hz, 1H), 7.52 (d, J = |
| | | [(3,3,3- | | 7.9 Hz, 2H), 7.46-7.34 (m, 3H), |
| | | trifluoropropyl)amino] | | 7.30-7.21 (m, 3H), 7.12 (d, J = 7.2 |
| | | pyrrolo[2,3-d]pyrimidin-5- | | Hz, 1H), 5.98 (s, 1H), 4.55-4.53 (m, |
| | | yl]phenyl)methyl]piperazin- | | 1H), 3.75-3.60 (m, 11H), 3.46 (s, |
| | | 1- | | 2H), 3.30 (d, J = 7.4 Hz, 2H), 3.14 |
| | | yl]ethoxy)ethoxy]propyl] | | (m, 1H), 3.05-2.96 (m, 1H), 2.89- |
| | | pyrido[2,3-b]indol-9- | | 2.79 (m, 1H), 2.67-2.38 (m, 11H), |
| | | yl)piperidine-2,6-dione | | 2.23-2.11 (m, 3H), 2.08-2.04 (m, |
| | | | | 6H), 1.61-1.48 (m, 2H), 1.31 (s, |
| | | | | 1H) |
| 134 | I-116 | 3-[3-methyl-2-oxo-5-[3- | 857.35 | (400 MHz, CD₃OD) δ 8.75 (s, 1H), |
| | | (3-[4-[(4-[7-[trans-4- | | 7.61 (d, J = 7.9 Hz, 2H), 7.44 (s, |
| | | hydroxycyclohexyl]-2- | | 1H), 7.39 (d, J = 7.9 Hz, 2H), 7.08- |
| | | [(3,3,3- | | 7.00 (m, 2H), 6.97 (d, J = 8.4 Hz, |
| | | trifluoropropyl)amino]- | | 1H), 5.32 (m, 1H), 4.55 (q, J = 8.2, |
| | | 7H-pyrrolo[2,3- | | 7.8 Hz, 1H), 3.79-3.68 (m, 3H), |
| | | d]pyrimidin-5- | | 3.58 (d, J = 6.9 Hz, 4H), 3.43 (s, |
| | | yl]phenyl)methyl]piperazin- | | 3H), 3.03 (p, J = 6.9 Hz, 1H), 2.99- |
| | | 1-yl]azetidin-1- | | 2.88 (m, 3H), 2.88-2.77 (m, 2H), |
| | | yl)propyl]-2,3-dihydro- | | 2.71 (t, J = 7.4 Hz, 2H), 2.65-2.27 |
| | | 1H-1,3-benzodiazol-1- | | (m, 11H), 2.22-2.04 (m, 7H), 1.73 |
| | | yl]piperidine-2,6-dione | | (p, J = 7.7 Hz, 2H), 1.57-1.55 (m, |
| | | | | 2H), 1.31 (s, 1H) |
| 135 | I-117 | 3-[3-methyl-2-oxo-5-[3- | 885.35 | (400 MHz, CD₃OD) δ 8.76 (s, 1H), |
| | | (4-[4-[(4-[7-[trans-4- | | 7.62 (d, J = 8.0 Hz, 2H), 7.44 (s, |
| | | hydroxycyclohexyl]-2- | | 1H), 7.40 (d, J = 8.0 Hz, 2H), 7.09- |
| | | [(3,3,3- | | 7.01 (m, 2H), 7.01-6.95 (m, 1H), |
| | | trifluoropropyl)amino]- | | 5.32 (m, 1H), 4.56 (p, J = 8.4 Hz, |
| | | 7H-pyrrolo[2,3- | | 1H), 3.73 (q, J = 8.5, 7.4 Hz, 3H), |
| | | d]pyrimidin-5- | | 3.57 (s, 2H), 3.43 (s, 3H), 3.05-3.03 |
| | | yl]phenyl)methyl]piperazin- | | (m, 2H), 2.96-2.88 (m, 1H), 2.87- |
| | | 1-yl]piperidin-1- | | 2.78 (m, 2H), 2.76-2.52 (m, 11H), |
| | | yl)propyl]-2,3-dihydro- | | 2.39-2.37 (m, 2H), 2.30-1.97 (m, |
| | | 1H-1,3-benzodiazol-1- | | 11H), 1.92-1.87 (m, 4H), 1.59-1.54 |
| | | yl]piperidine-2,6-dione | | (m, 4H) |
| 136 | I-118 | 3-[3-methyl-2-oxo-5-[3- | 885.35 | (400 MHz, CD₃OD) δ 8.76 (s, 1H), |
| | | (3-[4-[(4-[7-[trans-4- | | 8.49 (s, 1H), 7.64 (d, J = 7.9 Hz, |
| | | hydroxycyclohexyl]-2- | | 2H), 7.46 (s, 1H), 7.41 (d, J = 7.9 |
| | | [(3,3,3-trifluoropropyl) | | Hz, 2H), 7.12-7.04 (m, 2H), 7.04- |
| | | amino]-7H- | | 6.97 (m, 1H), 5.34 (dd, J = 12.4, 5.4 |
| | | pyrrolo[2,3-d]pyrimidin-5- | | Hz, 1H), 4.61-4.52 (m, 1H), 3.74 |
| | | yl]phenyl)methyl]piperazin- | | (q, J = 7.1 Hz, 3H), 3.68 (s, 2H), |
| | | 1-yl]piperidin-1- | | 3.44 (s, 3H), 3.30-3.20 (m, 5H), |
| | | yl)propyl]-2,3-dihydro- | | 2.97-2.89 (m, 3H), 2.82-2.75 (m, |
| | | 1H-1,3-benzodiazol-1- | | 10H), 2.71-2.56 (m, 6H), 2.22-2.01 |
| | | yl]piperidine-2,6-dione; | | (m, 9H), 1.97 (d, J = 11.3 Hz, 2H), |
| | | formic acid | | 1.69 (d, J = 9.5 Hz, 1H), 1.59 (d, J = |
| | | | | 10.2 Hz, 2H), 1.54 (d, J = 10.1 |
| | | | | Hz, 1H) |
| 137 | I-122 | 3-[3-methyl-2-oxo-5-[3- | 871.35 | (400 MHz, CD₃OD) δ 8.76 (s, 1H), |
| | | (3-[4-[(4-[trans-4- | | 8.43 (s, 1H), 7.65 (d, J = 8.0 Hz, |
| | | hydroxycyclohexyl]-2- | | 2H), 7.49-7.39 (m, 3H), 7.11-6.97 |
| | | [(3,3,3- | | (m, 3H), 5.34 (dd, J = 12.3, 5.4 Hz, |
| | | trifluoropropyl)amino]- | | 1H), 4.62-4.49 (m, 1H), 3.79-3.69 |
| | | 7H-pyrrolo[2,3- | | (m, 5H), 3.44 (s, 5H), 3.27-3.03 (m, |
| | | d]pyrimidin-5- | | 5H), 3.01-2.80 (m, 3H), 2.79 (d, J = |
| | | yl]phenyl)methyl]piperazin- | | 6.4 Hz, 3H), 2.68 (s, 3H), 2.60 |
| | | 1-yl]pyrrolidin-1- | | (dt, J = 11.2, 7.3 Hz, 5H), 2.25-2.11 |
| | | yl)propyl]-2,3-dihydro- | | (m, 4H), 2.06 (dd, J = 15.1, 7.5 Hz, |
| | | 1H-1,3-benzodiazol-1- | | 7H), 1.56 (q, J = 9.9 Hz, 2H) |
| | | yl]piperidine-2,6-dione; | | |
| | | formic acid | | |
| 138 | I-119 | 3-[3-methyl-2-oxo-5-[4- | 871.35 | (400 MHz, CD₃OD) δ 8.75 (s, 1H), |
| | | (3-[4-[(4-[7-[trans-4- | | 8.47 (s, 1H), 7.63 (d, J = 8.0 Hz, |
| | | hydroxycyclohexyl]-2- | | 2H), 7.45 (s, 1H), 7.41 (d, J = 8.1 |
| | | [(3,3,3- | | Hz, 2H), 7.08-7.01 (m, 2H), 6.98 |
| | | trifluoropropyl)amino]- | | (d, J = 8.0 Hz, 1H), 5.33 (dd, J = |
| | | 7H-pyrrolo[2,3- | | 12.5, 5.4 Hz, 1H), 4.60-4.58 (m, |
| | | d]pyrimidin-5- | | 1H), 3.82-3.79 (m, 5H), 3.42-3.37 |
| | | yl]phenyl)methyl]piperazin- | | (m, 5H), 3.29-3.11 (m, 5H), 2.97- |
| | | 1-yl]azetidin-1- | | 2.89 (m, 1H), 2.89-2.73 (m, 5H), |
| | | yl)butyl]-2,3-dihydro- | | 2.60-2.47 (m, 9H), 2.25-1.89 (m, |
| | | 1H-1,3-benzodiazol-1- | | 11H), 1.57-1.55 (m, 2H) |
| | | yl]piperidine-2,6-dione; | | |
| | | bis(formic acid) | | |
| 139 | I-123 | 3-[3-methyl-2-oxo-5-[4- | 885.35 | (400 MHz, CD₃OD) δ 8.77 (s, 1H), |
| | | (3-[4-(4-[7-[trans-4- | | 8.41 (s, 2H), 7.68 (d, J = 7.7 Hz, |
| | | hydroxycyclohexyl]-2- | | 2H), 7.51-7.43 (m, 3H), 7.08-7.01 |
| | | [(3,3,3- | | (m, 2H), 6.98 (d, J = 8.1 Hz, 1H), |
| | | trifluoropropyl)amino]- | | 5.32 (dd, J = 12.3, 5.4 Hz, 1H), 4.56 |
| | | 7H-pyrrolo[2,3- | | (p, J = 8.2 Hz, 1H), 3.89 (s, 2H), |
| | | d]pyrimidin-5- | | 3.73 (dd, J = 9.0, 5.6 Hz, 3H), 3.44- |
| | | yl]phenyl)methyl]piperazin- | | 3.42 (m, 5H), 3.30-3.08 (m, 5H), |
| | | 1-yl]pyrrolidin-1- | | 3.00-2.87 (m, 3H), 2.86 (s, 1H), |
| | | yl)butyl]-2,3-dihydro- | | 2.85-2.73 (m, 7H), 2.63-2.59 (m, |
| | | 1H-1,3-benzodiazol-1- | | 4H), 2.30-2.00 (m, 9H), 1.75-1.73 |
| | | yl]piperidine-2,6-dione; | | (m, 4H), 1.62-1.50 (m, 2H) |
| | | formic acid | | |
| 140 | I-120 | 3-[3-methyl-2-oxo-5-[4- | 899.35 | (400 MHz, DMSO-d₆) δ 11.09 (s, |
| | | (4-[4-[(4-[7-[trans-4- | | 1H), 8.88 (s, 1H), 7.66-7.60 (m, |
| | | hydroxycyclohexyl]-2- | | 3H), 7.30 (d, J = 8.0 Hz, 2H), 7.06 |
| | | [(3,3,3- | | (t, J = 5.8 Hz, 1H), 7.03-6.97 (m, |
| | | trifluoropropyl)amino]- | | 2H), 6.86 (dd, J = 8.1, 1.5 Hz, 1H), |
| | | 7H-pyrrolo[2,3- | | 5.34 (dd, J = 12.7, 5.4 Hz, 1H), 4.71 |
| | | d]pyrimidin-5- | | (d, J = 4.4 Hz, 1H), 4.42 (dt, J = |
| | | yl]phenyl)methyl]piperazin- | | 11.3, 6.2 Hz, 1H), 3.58-3.56 (m, |
| | | 1-yl]piperidin-1- | | 3H), 3.44 (s, 2H), 3.32 (s, 3H), |
| | | yl)butyl]-2,3-dihydro- | | 2.91-2.89 (m, 3H), 2.72-2.55 (m, |
| | | 1H-1,3-benzodiazol-1- | | 7H), 2.92-2.20 (m, 8H), 2.13-1.75 |
| | | yl]piperidine-2,6-dione | | (m, 10H), 1.72 (d, J = 11.8 Hz, 2H), |
| | | | | 1.57 (q, J = 7.8, 7.3 Hz, 2H), 1.48- |
| | | | | 1.29 (m, 6H) |
| 141 | I-121 | 3-[3-methyl-2-oxo-5-[4- | 899.35 | (400 MHz, DMSO-d₆) δ 11.10 (s, |
| | | (3-[4-[(4-[7-[trans-4- | | 1H), 8.88 (s, 1H), 7.66-7.60 (m, |
| | | hydroxycyclohexyl]-2- | | 3H), 7.30 (d, J = 7.8 Hz, 2H), 7.04- |
| | | [(3,3,3- | | 7.02 (m, 3H), 6.86 (d, J = 8.0 Hz, |
| | | trifluoropropyl)amino]- | | 1H), 5.34 (dd, J = 12.8, 5.4 Hz, 1H), |
| | | 7H-pyrrolo[2,3- | | 4.71 (d, J = 4.4 Hz, 1H), 4.41 (d, J = |
| | | d]pyrimidin-5- | | 11.8 Hz, 1H), 3.57 (q, J = 7.0 Hz, |
| | | yl]phenyl)methyl]piperazin- | | 3H), 3.43 (s, 2H), 3.32 (s, 3H), |
| | | 1-yl]piperidin-1- | | 2.95-2.85 (m, 2H), 2.75-2.57 (m, |
| | | yl)butyl]-2,3-dihydro- | | 8H), 2.35-2.29 (m, 10H), 2.03-1.94 |
| | | 1H-1,3-benzodiazol-1- | | (m, 7H), 1.81-1.55 (m, 6H), 1.45- |
| | | yl]piperidine-2,6-dione | | 1.28 (m, 5H), 1.11 (t, J = 13.9 Hz, |
| | | | | 1H) |
| 142 | I-129 | 3-(3-methyl-2-oxo-5-[2- | 887.45 | (400 MHz, CD₃OD) δ 8.77 (s, 1H), |
| | | [2-(3-[4-[(4-[7-[trans-4- | | 8.44 (s, 1H), 7.65 (d, J = 8.0 Hz, |
| | | hydroxycyclohexyl]-2- | | 2H), 7.47 (s, 1H), 7.42 (d, J = 8.0 |
| | | [(3,3,3- | | Hz, 2H), 7.11 (s, 1H), 7.03 (d, J = |
| | | trifluoropropyl)amino]- | | 1.0 Hz, 2H), 5.30 (dd, J = 12.4, 5.4 |
| | | 7H-pyrrolo[2,3- | | Hz, 1H), 4.63-4.53 (m, 1H), 3.92- |
| | | d]pyrimidin-5- | | 3.86 (m, 2H), 3.79-3.64 (m, 11H), |
| | | yl]phenyl)methyl]piperazin- | | 3.44 (s, 3H), 3.27 (t, J = 4.9 Hz, |
| | | 1-yl]azetidin-1- | | 2H), 3.17 (d, J = 6.8 Hz, 1H), 2.97 |
| | | yl)ethoxy]ethyl]-2,3- | | (t, J = 6.3 Hz, 2H), 2.89-2.87 (m, |
| | | dihydro-1H-1,3- | | 1H), 2.82-2.73 (m, 2H), 2.68-2.55 |
| | | benzodiazol-1- | | (m, 5H), 2.36-2.34 (m, 3H), 2.20- |
| | | yl)piperidine-2,6-dione; | | 2.07 (m, 7H), 2.06-2.04 (m, 2H), |
| | | formic acid | | 1.56-1.54 (m, 2H) |
| 143 | I-132 | 3-(3-methyl-2-oxo-5-[2- | 901.45 | (400 MHz, CD₃OD) δ 8.77 (s, 1H), |
| | | [2-(3-[4-[(4-[7-[trans-4- | | 7.62 (d, J = 8.0 Hz, 2H), 7.45 (s, |
| | | hydroxycyclohexyl]-2- | | 1H), 7.40 (d, J = 8.0 Hz, 2H), 7.10 |
| | | [(3,3,3- | | (s, 1H), 7.01 (s, 2H), 5.35-5.25 (m, |
| | | trifluoropropyl)amino]- | | 1H), 4.58-4.56 (m, 1H), 3.74-2.70 |
| | | 7H-pyrrolo[2,3- | | (m, 5H), 3.59-3.56 (m, 4H), 3.43 (s, |
| | | d]pyrimidin-5- | | 3H), 2.96-2.93 (m, 4H), 2.87-2.75 |
| | | yl]phenyl)methyl]piperazin- | | (m, 5H), 2.73-2.53 (m, 8H), 2.47- |
| | | 1-yl]pyrrolidin-1- | | 2.45 (m, 3H), 2.29 (d, J = 9.4 Hz, |
| | | yl)ethoxy]ethyl]-2,3- | | 1H), 2.20-2.04 (m, 9H), 1.97-1.95 |
| | | dihydro-1H-1,3- | | (m, 1H), 1.68-1.50 (m, 3H) |
| | | benzodiazol-1- | | |
| | | yl)piperidine-2,6-dione | | |
| 144 | I-130 | 3-(3-methyl-2-oxo-5-[2- | 915.50 | (400 MHz, CD₃OD) δ 8.77 (s, 1H), |
| | | [2-(4-[4-[(4-[7-[trans-4- | | 7.69-7.56 (m, 2H), 7.45 (s, 1H), |
| | | hydroxycyclohexyl]-2- | | 7.40 (d, J = 7.9 Hz, 2H), 7.10 (s, |
| | | [(3,3,3- | | 1H), 7.02 (s, 2H), 5.33 (dd, J = |
| | | trifluoropropyl)amino]- | | 12.3, 5.4 Hz, 1H), 4.60-4.52 (m, |
| | | 7H-pyrrolo[2,3- | | 1H), 3.73-3.69 (m, 5H), 3.61-3.55 |
| | | yl]phenyl)methyl]piperazin- | | (m, 4H), 3.44 (s, 3H), 2.97-2.80 |
| | | 1-yl]piperidin-1- | | (m, 8H), 2.7-2.53 (m, 10H), 2.16- |
| | | yl)ethoxy]ethyl]-2,3- | | 1.98 (m, 11H), 1.79 (d, J = 12.5 Hz, |
| | | dihydro-1H-1,3- | | 2H), 1.56-1.51 (m, 4H) |
| | | benzodiazol-1- | | |
| | | yl)piperidine-2,6-dione | | |
| 145 | I-131 | 3-(3-methyl-2-oxo-5-[2- | 915.50 | (400 MHz, CD₃OD) δ 8.75 (d, J = |
| | | [2-(3-[4-[(4-[7-[trans-4- | | 1.5 Hz, 1H), 8.48 (s, 1H), 7.64 (d, J = |
| | | hydroxycyclohexyl]-2- | | 8.0 Hz, 2H), 7.46 (s, 1H), 7.41 (d, |
| | | [(3,3,3- | | J = 7.7 Hz, 2H), 7.09 (s, 1H), 7.02 |
| | | trifluoropropyl)amino]- | | (s, 2H), 5.32 (d, J = 6.9 Hz, 1H), |
| | | 7H-pyrrolo[2,3- | | 4.56 (d, J = 7.8 Hz, 1H), 3.79-3.71 |
| | | d]pyrimidin-5- | | (m, 6H), 3.70-3.68 (m, 3H), 3.40 (s, |
| | | yl]phenyl)methyl]piperazin- | | 3H), 3.05-2.87 (m, 7H), 2.85-2.76 |
| | | 1-yl]piperidin-1- | | (m, 3H), 2.75-2.54 (m, 12H), 2.16- |
| | | yl)ethoxy]ethyl]-2,3- | | 2.10 (m, 3H), 2.09-2.06 (m, 4H), |
| | | dihydro-1H-1,3- | | 1.85 (d, J = 11.1 Hz, 2H), 1.56-1.54 |
| | | benzodiazol-1- | | (m, 4H) |
| | | yl)piperidine-2,6-dione; | | |
| | | formic acid | | |
| 146 | I-125 | 3-(6-[3-[2-(2-[4-[(4-[7- | 910.35 | (400 MHz, CD₃OD) δ 8.72 (s, 1H), |
| | | [trans-4- | | 8.42 (dd, J = 7.6, 1.6 Hz, 1H), 8.37 |
| | | hydroxycyclohexyl]-2- | | (dd, J = 4.9, 1.6 Hz, 1H), 7.99 (d, J = |
| | | [(3,3,3- | | 1.6 Hz, 1H), 7.57-7.50 (m, 2H), |
| | | trifluoropropyl)amino]- | | 7.44-7.34 (m, 3H), 7.34-7.28 (m, |
| | | 7H-pyrrolo[2,3- | | 2H), 7.21 (dd, J = 7.7, 4.9 Hz, 1H), |
| | | d]pyrimidin-5- | | 5.95 (d, J = 9.8 Hz, 1H), 4.53 (p, J = |
| | | yl]phenyl)methyl]piperazin- | | 8.2 Hz, 1H), 3.79-3.58 (m, 10H), |
| | | 1- | | 3.56-3.47 (m, 4H), 3.19-2.95 (m, |
| | | yl]ethoxy)ethoxy]propyl]- | | 3H), 2.91-2.82 (m, 3H), 2.76-2.46 |
| | | 9H-pyrido[2,3-b]indol- | | (m, 11H), 2.26-1.91 (m, 10H), |
| | | 9-yl)piperidine-2,6-dione | | 1.54-1.52 (m, 2H), 1.31 (s, 1H) |
| 147 | I-126 | 3-(4-[3-[2-(2-[4-[(4-[7- | 910.55 | (400 MHz, CD₃OD) δ 8.74 (s, 1H), |
| | | [trans-4- | | 8.32-8.21 (m, 2H), 7.58-7.47 (m, |
| | | hydroxycyclohexyl]-2- | | 4H), 7.41 (s, 1H), 7.35-7.24 (m, |
| | | [(3,3,3- | | 3H), 7.09 (d, J = 5.1 Hz, 1H), 5.99 |
| | | trifluoropropyl)amino]- | | (s, 1H), 4.55 (p, J = 8.5 Hz, 1H), |
| | | 7H-pyrrolo[2,3- | | 3.79-3.56 (m, 12H), 3.48 (d, J = 4.3 |
| | | d]pyrimidin-5- | | Hz, 2H), 3.14-2.96 (m, 2H), 2.87- |
| | | yl]phenyl)methyl]piperazin- | | 2.80 (m, 1H), 2.77-2.42 (m, 12H), |
| | | 1- | | 2.24-1.96 (m, 10H), 1.54 (m, 2H), |
| | | yl]ethoxy)ethoxy]propyl]- | | 1.31 (q, J = 8.4, 7.7 Hz, 1H) |
| | | 9H-pyrido[2,3-b]indol- | | |
| | | 9-yl)piperidine-2,6-dione | | |
| 148 | I-127 | 3-(7-[3-[2-(2-[4-[(4-[7- | 910.45 | (400 MHz, CD₃OD) δ 8.71 (s, 1H), |
| | | [trans-4- | | 8.35 (d, J = 6.3 Hz, 2H), 8.01 (d, J = |
| | | hydroxycyclohexyl]-2- | | 8.0 Hz, 1H), 7.54 (d, J = 7.9 Hz, |
| | | [(3,3,3- | | 2H), 7.41-7.30 (m, 4H), 7.22-7.10 |
| | | trifluoropropyl)amino]- | | (m, 2H), 5.97 (d, J = 11.2 Hz, 1H), |
| | | 7H-pyrrolo[2,3- | | 4.57-4.49 (m, 1H), 3.74-3.59 (m, |
| | | d]pyrimidin-5- | | 9H), 3.53-3.51 (m, 4H), 3.19-2.96 |
| | | yl]phenyl)methyl]piperazin- | | (m, 3H), 2.92-2.83 (m, 3H), 2.73- |
| | | 1- | | 2.52 (m, 11H), 2.16-1.93 (m, 9H), |
| | | yl]ethoxy)ethoxy]propyl]- | | 1.55-1.53 (m, 2H), 1.34-1.28 (m, |
| | | 9H-pyrido[2,3-b]indol- | | 1H) |
| | | 9-yl)piperidine-2,6-dione | | |
| 149 | I-138 | 3-[3-methyl-2-oxo-4-[3- | 860.35 | (400 MHz, CD₃OD) δ 8.76 (s, 1H), |
| | | (3-[4-[(4-[7-[trans-4- | | 7.62 (d, J = 8.1 Hz, 2H), 7.47-7.37 |
| | | hydroxycyclohexyl]-2- | | (m, 3H), 7.09-7.00 (m, 1H), 6.97 (t, |
| | | [(3,3,3- | | J = 7.2 Hz, 2H), 5.33 (m, 1H), 4.57 |
| | | trifluoropropyl)amino] | | (p, J = 8.2 Hz, 1H), 3.75-3.73 (m, |
| | | pyrrolo[2,3-d]pyrimidin-5- | | 3H), 3.69 (s, 3H), 3.59 (s, 2H), |
| | | yl]phenyl)methyl]piperazin- | | 3.51-3.49 (m, 4H), 3.12-3.04 (m, |
| | | 1-yl]propoxy)propyl]- | | 2H), 2.98-2.77 (m, 4H), 2.70-2.40 |
| | | 1,3-benzodiazol-1- | | (m, 11H), 2.21-2.04 (m, 7H), 1.93 |
| | | yl]piperidine-2,6-dione | | (p, J = 6.1 Hz, 2H), 1.81 (p, J = 6.3 |
| | | | | Hz, 2H), 1.57-1.55 (m, 2H) |
| 150 | I-136 | 3-[3-methyl-2-oxo-5-[3- | 856.45 | ¹H NMR (400 MHz, DMSO-d₆) δ |
| | | (3-[1-[(4-[7-[trans-4- | | 11.09 (s, 1H), 8.88 (s, 1H), 8.24 (s, |
| | | hydroxycyclohexyl]-2- | | 3H), 7.68-7.58 (m, 3H), 7.30 (d, J = |
| | | [(3,3,3- | | 8.0 Hz, 2H), 7.10-6.97 (m, 3H), |
| | | trifluoropropyl)amino] | | 6.86 (dd, J = 8.1, 1.5 Hz, 1H), 5.34 |
| | | pyrrolo[2,3-d]pyrimidin-5- | | (m, 1H), 4.43 (t, J = 7.4 Hz, 1H), |
| | | yl]phenyl)methyl]piperidin- | | 3.64-3.38 (m, 8H), 3.02 (s, 2H), |
| | | 4-yl]azetidin-1- | | 2.93-2.78 (m, 3H), 2.75-2.67 (m, |
| | | yl)propyl]-1,3- | | 1H), 2.62-2.58 (m, 10H), 2.20 (q, J = |
| | | benzodiazol-1- | | 7.9 Hz, 1H), 2.05-1.85 (m, 9H), |
| | | yl]piperidine-2,6-dione; | | 1.67-1.50 (m, 4H), 1.38-1.36 (m, |
| | | tris(formic acid) | | 3H), 1.05-1.03 (m, 2H) |
| 151 | I-137 | 3-[3-methyl-2-oxo-5-(3- | 884.45 | (400 MHz, DMSO-d₆) δ 11.08 (s, |
| | | [1-[(4-[7-[trans-4- | | 1H), 8.88 (s, 1H), 7.63 (d, J = 6.7 |
| | | hydroxycyclohexyl]-2- | | Hz, 3H), 7.30 (d, J = 7.7 Hz, 2H), |
| | | [(3,3,3- | | 7.10-6.96 (m, 3H), 6.87 (d, J = 8.0 |
| | | trifluoropropyl)amino] | | Hz, 1H), 5.34 (m, 1H), 4.70 (d, J = |
| | | pyrrolo[2,3-d]pyrimidin-5- | | 4.4 Hz, 1H), 4.43 (p, J = 7.4, 6.4 |
| | | yl]phenyl)methyl]-[4,4- | | Hz, 1H), 3.59-3.56 (m, 3H), 3.45 (s, |
| | | bipiperidin]-1-yl]propyl)- | | 2H), 3.38 (s, 3H), 2.89 (m, 5H), |
| | | 1,3-benzodiazol-1- | | 2.73-2.59 (m, 6H), 2.26-2.24 (s, |
| | | yl]piperidine-2,6-dione | | 2H), 1.96-1.89 (m, 10H), 1.76-1.74 |
| | | | | (m, 3H), 1.65-1.62 (m, 4H), 1.37- |
| | | | | 1.35 (m, 2H), 1.25-1.11 (m, 4H), |
| | | | | 1.03 (s, 2H) |
| 152 | I-149 | 3-[3-methyl-2-oxo-5-[(3- | 832.45 | (400 MHz, CD₃OD) δ 8.76 (s, 1H), |
| | | [4-[(4-[7-[trans-4- | | 8.51 (s, 1H), 7.64 (d, J = 7.9 Hz, |
| | | hydroxycyclohexyl]-2- | | 2H), 7.46 (s, 1H), 7.40 (d, J = 7.9 |
| | | [(3,3,3- | | Hz, 2H), 7.21 (s, 1H), 7.17-7.08 (m, |
| | | trifluoropropyl)amino] | | 2H), 5.36 (m, 1H), 4.58 (s, 3H), |
| | | pyrrolo[2,3-d]pyrimidin-5- | | 3.80-3.56 (m, 7H), 3.44 (s, 3H), |
| | | yl]phenyl)methyl]piperazin- | | 3.13-2.75 (m, 10H), 2.74-2.52 (m, |
| | | 1-yl]propoxy)methyl]- | | 5H), 2.22-2.05 (m, 7H), 1.95 (p, J = |
| | | 1,3-benzodiazol-1- | | 6.3 Hz, 2H), 1.56-1.54 (m, 2H) |
| | | yl]piperidine-2,6-dione; | | |
| | | formic acid | | |
| 153 | I-151 | trans-3-[3-methyl-2-oxo- | 818.35 | (400 MHz, CD₃OD) δ 8.76 (s, 1H), |
| | | 5-[(2-[4-[(4-[7-[4- | | 7.61 (d, J = 7.9 Hz, 2H), 7.44 (s, |
| | | hydroxycyclohexyl]-2- | | 1H), 7.39 (d, J = 7.8 Hz, 2H), 7.22 |
| | | [(3,3,3- | | (s, 1H), 7.16-7.06 (m, 2H), 5.34 (m, |
| | | trifluoropropyl)amino] | | 1H), 4.58 (s, 3H), 3.79-3.69 (m, |
| | | pyrrolo[2,3-d]pyrimidin-5- | | 3H), 3.64 (t, J = 5.6 Hz, 2H), 3.57 |
| | | yl]phenyl)methyl]piperazin- | | (s, 2H), 3.45 (s, 3H), 2.99-2.73 (m, |
| | | 1-yl]ethoxy)methyl]- | | 4H), 2.66-2.58 (m, 11H), 2.22-2.02 |
| | | 1,3-benzodiazol-1- | | (m, 8H), 1.56-1.54 (m, 2H) |
| | | yl]piperidine-2,6-dione; | | |
| | | formic acid | | |
| 154 | I-150 | 3-[3-methyl-2-oxo-4-[(3- | 832.45 | (400 MHz, CD₃OD) δ 8.76 (s, 1H), |
| | | [4-[(4-[7-[trans-4- | | 7.61 (d, J = 8.0 Hz, 2H), 7.44 (s, |
| | | hydroxycyclohexyl]-2- | | 1H), 7.39 (d, J = 8.0 Hz, 2H), 7.17- |
| | | [(3,3,3- | | 7.05 (m, 3H), 5.35 (m, 1H), 4.77 (s, |
| | | trifluoropropyl)amino] | | 2H), 4.60-4.52 (m, 1H), 3.73-3.70 |
| | | pyrrolo[2,3-d]pyrimidin-5- | | (m, 6H), 3.58-3.55 (m, 4H), 2.95- |
| | | yl]phenyl)methyl]piperazin- | | 2.75 (m, 3H), 2.65-2.40 (m, 11H), |
| | | 1-yl]propoxy)methyl]- | | 2.19-2.06 (m, 7H), 1.80 (t, J = 7.7 |
| | | 1,3-benzodiazol-1- | | Hz, 2H), 1.56-1.54 (m, 2H) |
| | | yl]piperidine-2,6-dione | | |
| 155 | I-152 | 3-[3-methyl-2-oxo-4-[(2- | 818.40 | (400 MHz, CD₃OD) δ 8.77 (s, 1H), |
| | | [4-[(4-[7-[trans-4- | | 8.49 (s, 2H), 7.65 (d, J = 7.9 Hz, |
| | | hydroxycyclohexyl]-2- | | 2H), 7.46 (s, 1H), 7.42 (d, J = 8.0 |
| | | [(3,3,3- | | Hz, 2H), 7.16-7.07 (m, 3H), 5.36 |
| | | trifluoropropyl)amino] | | (dd, J = 11.8, 5.5 Hz, 1H), 4.85- |
| | | pyrrolo[2,3-d]pyrimidin-5- | | 4.83 (m, 4H), 4.61-4.53 (m, 1H), |
| | | yl]phenyl)methyl]piperazin- | | 3.71 (m, 10H), 2.92-2.70 (m, 11H), |
| | | 1-yl]ethoxy)methyl]- | | 2.63-2.60 (m, 2H), 2.18-2.06 (m, |
| | | 1,3-benzodiazol-1- | | 7H), 1.61-1.50 (m, 2H), 1.31 (s, |
| | | yl]piperidine-2,6-dione; | | 1H) |
| | | bis(formic acid) | | |
| 156 | I-160 | 3-[5-[3-(2-fluoro-3-[4- | 878.45 | (400 MHz, CD₃OD) δ 8.76 (s, 1H), |
| | | [(4-[7-[trans-4- | | 7.63 (d, J = 8.2 Hz, 2H), 7.47-7.38 |
| | | hydroxycyclohexyl]-2- | | (m, 3H), 7.08-6.94 (m, 3H), 5.32 |
| | | [(3,3,3- | | (dd, J = 12.4, 5.4 Hz, 1H), 4.83- |
| | | trifluoropropyl)amino] | | 4.72 (m, 1H), 4.60-4.52 (m, 2H), |
| | | pyrrolo[2,3-d]pyrimidin-5- | | 3.79-3.69 (m, 3H), 3.67-3.55 (m, |
| | | yl]phenyl)methyl]piperazin- | | 5H), 3.54-3.46 (m, 2H), 3.43 (s, |
| | | 1-yl]propoxy)propyl]- | | 3H), 3.00-2.65 (m, 5H), 2.65-2.62 |
| | | 3-methyl-2-oxo-1,3- | | (m, 5H), 2.64-2.54 (m, 10H), 2.14- |
| | | benzodiazol-1- | | 2.05 (m, 6H), 1.94 (q, J = 6.8 Hz, |
| | | yl]piperidine-2,6-dione | | 2H), 1.62-1.50 (m, 2H), 1.20 (t, J = |
| | | | | 7.0 Hz, 2H) |
| 157 | I-220 | 3-(3-methyl-2-oxo-5- | 881.55 | (400 MHz, CD₃OD) δ 8.41 (s, 4H), |
| | | [[(3S)-3-[3-(4-[[4-[2- | | 7.22 (s, 1H), 7.15-7.05 (m, 2H), |
| | | [(2S)-pentan-2-ylamino]- | | 6.84 (s, 1H), 5.35 (m, 1H), 4.61- |
| | | 7-[trans-4- | | 4.04 (m, 5H), 3.84-3.81 (m, 1H), |
| | | hydroxycyclohexyl] | | 3.48-3.37 (m, 5H), 3.35 (s, 3H), |
| | | pyrrolo[2,3-d]pyrimidin-5- | | 3.01-2.69 (m, 6H), 2.65-2.34 (m, |
| | | yl]cyclohexyl]methyl] | | 13H), 2.20-2.16 (m, 2H), 2.14-2.06 |
| | | piperazin-1- | | (m, 3H), 2.06-1.91 (m, 5H), 1.80- |
| | | yl)propoxy]pyrrolidin-1- | | 1.75 (m, 6H), 1.66-1.63 (m, 5H), |
| | | yl]methyl]-1,3- | | 1.50-1.46 (m, 6H), 1.25-1.23 (m, |
| | | benzodiazol-1- | | 3H), 0.99-0.97 (m, 3H) |
| | | yl)piperidine-2,6-dione | | |
| 158 | I-211 | 3-(3-methyl-2-oxo-5- | 881.55 | (400 MHz, CD₃OD) δ 8.42 (s, 1H), |
| | | [[(3S)-3-[3-(4-[[trans-4- | | 7.23 (d, J = 1.5 Hz, 1H), 7.11 (q, J = |
| | | [2-[(2S)-pentan-2- | | 8.1 Hz, 2H), 6.81 (s, 1H), 5.35 |
| | | ylamino]-7-[trans-4- | | (dd, J = 12.4, 5.4 Hz, 1H), 4.43 (p, |
| | | hydroxycyclohexyl] | | J = 8.4, 8.0 Hz, 1H), 4.08 (dt, J = |
| | | pyrrolo[2,3-d]pyrimidin-5- | | 13.3, 6.7 Hz, 2H), 3.78 (d, J = 12.5 |
| | | yl]cyclohexyl]methyl] | | Hz, 1H), 3.69 (s, 1H), 3.66 (s, 1H), |
| | | piperazin-1- | | 3.48-3.44 (m, 5H), 3.01-2.73 (m, |
| | | yl)propoxy]pyrrolidin-1- | | 6H), 2.72-2.35 (m, 12H), 2.25 (d, J = |
| | | yl]methyl]-1,3- | | 6.8 Hz, 2H), 2.22-2.01 (m, 7H), |
| | | benzodiazol-1- | | 2.01-1.90 (m, 6H), 1.86-1.71 (m, |
| | | yl)piperidine-2,6-dione | | 3H), 1.71-1.59 (m, 2H), 1.58-1.37 |
| | | | | (m, 7H), 1.25-1.23 (m, 3H), 1.14 |
| | | | | (q, J = 12.4 Hz, 2H), 0.98 (t, J = 7.1 |
| | | | | Hz, 3H) |
| 159 | I-219 | 3-(3-methyl-2-oxo-5- | 881.55 | (400 MHz, CD₃OD) δ 8.46-8.42 |
| | | [[(3S)-3-[3-(4-[[trans-4- | | (m, 4H), 7.35 (s, 1H), 7.29-7.17 (m, |
| | | [2-[(2S)-pentan-2- | | 2H), 6.89 (s, 1H), 5.39 (dd, J = |
| | | ylamino]-7-[trans-4- | | 12.6, 5.4 Hz, 1H), 4.49-4.40 (m, |
| | | hydroxycyclohexyl] | | 1H), 4.32 (d, J = 12.8 Hz, 1H), |
| | | pyrrolo[2,3-d]pyrimidin-5- | | 4.23-4.20 (m, 2H), 4.10 (d, J = 6.3 |
| | | yl]cyclohexyl]methyl] | | Hz, 1H), 3.69-3.67 (m, 1H), 3.60- |
| | | piperazin-1- | | 3.51 (m, 2H), 3.48 (s, 3H), 3.30- |
| | | yl)propoxy]pyrrolidin-1- | | 3.22 (m, 5H), 2.93-2.82 (m, 15H), |
| | | yl]methyl-1,3- | | 2.66 (d, J = 7.3 Hz, 2H), 2.28 (q, J = |
| | | benzodiazol-1- | | 6.8 Hz, 1H), 2.21 (t, J = 5.8 Hz, |
| | | yl)piperidine-2,6-dione; | | 1H), 2.12-2.08 (m, 3H), 2.00-1.97 |
| | | tris(formic acid) | | (m, 5H), 1.87-1.85 (m, 6H), 1.75 |
| | | | | (d, J = 14.6 Hz, 2H), 1.66-1.64 (m, |
| | | | | 3H), 1.49-1.47 (m, 6H), 1.26-1.24 |
| | | | | (m, 3H), 0.99 (t, J = 7.1 Hz, 3H) |
| 160 | I-221 | trans-3-(3-methyl-2-oxo- | 881.55 | (400 MHz, CD₃OD) δ 8.42 (s, 1H), |
| | | 5-[[(3R)-3-[3-(4- | | 7.25 (d, J = 1.4 Hz, 1H), 7.19-7.08 |
| | | [[(1r,4r)-4-[2-[(2S)- | | (m, 2H), 6.81 (s, 1H), 5.36 (m, 1H), |
| | | pentan-2-ylamino]-7-[4- | | 4.47-4.39 (m, 1H), 4.09 (m, 2H), |
| | | hydroxycyclohexyl] | | 3.91 (m, 1H), 3.80 (m, 1H), 3.68 |
| | | pyrrolo[2,3-d]pyrimidin-5- | | (m, 1H), 3.46 (s, 5H), 3.01-2.51 (m, |
| | | yl]cyclohexyl]methyl] | | 17H), 2.31 (d, J = 6.8 Hz, 2H), |
| | | piperazin-1- | | 2.24-2.13 (m, 2H), 2.08 (m, 4H), |
| | | yl)propoxy]pyrrolidine-1- | | 2.01-1.85 (m, 7H), 1.81 (p, J = 6.2 |
| | | yl]methyl]-1,3- | | Hz, 2H), 1.71-1.59 (m, 2H), 1.59- |
| | | benzodiazol-1- | | 1.35 (m, 7H), 1.19 (m, 5H), 0.98 (t, |
| | | yl)piperidine-2,6-dione | | J = 7.2 Hz, 3H) |
| 161 | I-222 | 3-[3-methyl-2-oxo-5-([3- | 867.80 | (400 MHz, CD₃OD) δ 8.41 (s, 1H), |
| | | [3-(4-[[cis-4-[2-[(2S)- | | 7.18 (s, 1H), 7.10 (d, J = 1.6 Hz, |
| | | pentan-2-ylamino]-7- | | 2H), 6.85 (s, 1H), 5.51 (s, 1H), |
| | | [trans-4- | | 5.40-5.33 (m, 1H), 4.48-4.41 (m, |
| | | hydroxycyclohexyl] | | 1H), 4.21-4.16 (m, 1H), 4.09 (q, J = |
| | | pyrrolo[2,3-d]pyrimidin-5- | | 6.5 Hz, 1H), 3.82 (s, 2H), 3.69 (d, |
| | | yl]cyclohexyl]methyl] | | J = 5.8 Hz, 3H), 3.62 (s, 1H), 3.47- |
| | | piperazin-1- | | 3.45 (m, 5H), 3.21 (s, 2H), 2.95- |
| | | yl)propoxy]azetidin-1- | | 2.78 (m, 5H), 2.62-2.58 (m, 8H), |
| | | yl]methyl)-1,3- | | 2.46 (s, 2H), 2.16-2.14 (m, 4H), |
| | | benzodiazol-1- | | 1.99-1.96 (m, 5H), 1.85-1.81 (m, |
| | | yl]piperidine-2,6-dione | | 5H), 1.74-1.58 (m, 6H), 1.56-1.43 |
| | | | | (m, 6H), 1.31 (s, 2H), 1.25-1.23 (m, |
| | | | | 3H), 0.98 (t, J = 7.1 Hz, 3H) |
| 162 | I-223 | 3-[3-methyl-2-oxo-5-([3- | 868.85 | (400 MHz, CD₃OD) δ 8.42 (s, 1H), |
| | | [3-(4-[[trans-4-[2-[(2S)- | | 7.16 (s, 1H), 7.09 (d, J = 1.5 Hz, |
| | | pentan-2-ylamino]-7- | | 2H), 6.81 (s, 1H), 5.34 (dd, J = |
| | | [trans-4- | | 12.4, 5.3 Hz, 1H), 4.42 (q, J = 7.7 |
| | | hydroxycyclohexyl] | | Hz, 1H), 4.13-4.11 (m, 2H), 3.75- |
| | | pyrrolo[2,3-d]pyrimidin-5- | | 3.67 (m, 3H), 3.60-3.58 (m, 2H), |
| | | yl]cyclohexyl]methyl] | | 3.46-3.42 (m, 5H), 3.09 (m, 2H), |
| | | piperazin-1- | | 2.96-2.89 (m, 1H), 2.86-2.78 (m, |
| | | yl)propoxy]azetidin-1- | | 2H), 2.69-2.67 (m, 2H), 2.55-2.45 |
| | | yl]methyl)-1,3- | | (m, 8H), 2.25 (d, J = 6.8 Hz, 2H), |
| | | benzodiazol-1- | | 2.22-2.15 (m, 1H), 2.11-2.07 (m, |
| | | yl]piperidine-2,6-dione | | 5H), 1.99-1.97 (m, 6H), 1.81-1.62 |
| | | | | (m, 4H), 1.57-1.39 (m, 8H), 1.31 (s, |
| | | | | 1H), 1.24 (d, J = 6.5 Hz, 3H), 1.14- |
| | | | | 1.12 (m, 2H), 0.98 (t, J = 7.0 Hz, |
| | | | | 3H) |
| 163 | I-197 | 3-(3-methyl-2-oxo-5-[[4- | 929.50 | (400 MHz, CD₃OD) δ 8.76 (s, 1H), |
| | | (3-[4-[(4-[7-[trans-4- | | 7.65-7.59 (m, 2H), 7.46-7.37 (m, |
| | | hydroxycyclohexyl]-2- | | 3H), 7.21 (s, 1H), 7.10 (d, J = 2.1 |
| | | [[(2S)-4,4,4- | | Hz, 2H), 5.35 (dd, J = 12.4, 5.4 Hz, |
| | | trifluorobutan-2- | | 1H), 4.59-4.46 (m, 2H), 3.78-3.69 |
| | | yl]amino]pyrrolo[2,3- | | (m, 2H), 3.68 (s, 1H), 3.60 (s, 2H), |
| | | d]pyrimidin-5- | | 3.52 (d, J = 6.1 Hz, 2H), 3.46-3.42 |
| | | yl]phenyl)methyl]piperazin- | | (m, 5H), 2.99-2.88 (m, 1H), 2.88- |
| | | 1- | | 2.77 (m, 4H), 2.68-2.46 (m, 9H), |
| | | yl]propoxy)piperidin-1- | | 2.37-2.35 (m, 3H), 2.24-2.02 (m, |
| | | yl]methyl]-1,3- | | 8H), 1.94-1.92 (m, 2H), 1.78 (t, J = |
| | | benzodiazol-1- | | 7.6 Hz, 2H), 1.69-1.51 (m, 4H), |
| | | yl)piperidine-2,6-dione | | 1.42 (d, J = 6.7 Hz, 3H) |
| 164 | I-196 | 3-(3-methyl-2-oxo-5-[[4- | 929.80 | (400 MHz, CD₃OD) δ 8.76 (s, 1H), |
| | | (3-[4-[(4-[7-[trans-4- | | 7.62 (d, J = 7.9 Hz, 2H), 7.46-7.37 |
| | | hydroxycyclohexyl]-2- | | (m, 3H), 7.20 (s, 1H), 7.09 (s, 2H), |
| | | [[(2R)-4,4,4- | | 5.34 (t, J = 6.1 Hz, 1H), 4.57 (s, |
| | | trifluorobutan-2- | | 1H), 4.48 (q, J = 6.6 Hz, 1H), 3.72 |
| | | yl]amino]pyrrolo[2,3- | | (d, J = 11.1 Hz, 1H), 3.63 (s, 2H), |
| | | d]pyrimidin-5- | | 3.59 (s, 2H), 3.53-3.51 (m, 3H), |
| | | yl]phenyl)methyl]piperazin- | | 3.45 (s, 3H), 2.95-2.71 (m, 7H), |
| | | 1- | | 2.56-2.50 (m, 8H), 2.37-2.25 (m, |
| | | yl]propoxy)piperidin-1- | | 3H), 2.13-2.08 (m, 7H), 1.98-1.87 |
| | | yl]methyl]-1,3- | | (m, 3H), 1.78 (d, J = 8.4 Hz, 2H), |
| | | benzodiazol-1- | | 1.60-1.58 (m, 4H), 1.42 (d, J = 6.7 |
| | | yl)piperidine-2,6-dione | | Hz, 3H) |
| 165 | I-224 | 1-methyl-3-[3-methyl-2- | 888.60 | (400 MHz, CD₃OD) δ 8.75 (s, 1H), |
| | | oxo-5-[3-(3-[4-[(4-[7- | | 7.62 (d, J = 8.2 Hz, 2H), 7.46-7.36 |
| | | [trans-4- | | (m, 3H), 7.05 (d, J = 1.4 Hz, 1H), |
| | | hydroxycyclohexyl]-2- | | 7.02-6.93 (m, 2H), 5.33 (m, 1H), |
| | | [[(2R)-4,4,4- | | 4.64-4.43 (m, 2H), 3.78-3.68 (m, |
| | | trifluorobutan-2- | | 1H), 3.59 (s, 2H), 3.51-3.39 (m, |
| | | yl]amino]pyrrolo[2,3- | | 7H), 3.18 (s, 3H), 2.99-2.90 (m, |
| | | d]pyrimidin-5- | | 2H), 2.84-2.68 (m, 5H), 2.67-2.47 |
| | | yl]phenyl)methyl]piperazin- | | (m, 8H), 2.36 (m, 2H), 2.19-2.00 |
| | | 1-yl]propoxy)propyl]- | | (m, 7H), 1.92-1.88 (m, 2H), 1.81- |
| | | 1,3-benzodiazol-1- | | 1.77 (m, 2H), 1.61-1.49 (m, 2H), |
| | | yl]piperidine-2,6-dione | | 1.42 (d, J = 6.7 Hz, 3H) |
| 166 | I-225 | 1-methyl-3-[3-methyl-2- | 888.35 | (400 MHz, CD₃OD) δ 8.75 (s, 1H), |
| | | oxo-5-[3-(3-[4-[(4-[7- | | 7.64-7.58 (m, 2H), 7.45-7.36 (m, |
| | | [trans-4- | | 3H), 7.07-7.02 (m, 1H), 7.02-6.92 |
| | | hydroxycyclohexyl]-2- | | (m, 2H), 5.32 (dd, J = 12.9, 5.5 Hz, |
| | | [[(2S)-4,4,4- | | 1H), 4.63-4.44 (m, 2H), 3.72 (m, |
| | | trifluorobutan-2- | | 1H), 3.57 (s, 2H), 3.51-3.40 (m, |
| | | yl]amino]pyrrolo[2,3- | | 7H), 3.30-3.23 (m, 1H), 3.18 (s, |
| | | d]pyrimidin-5- | | 3H), 2.98-2.87 (m, 2H), 2.84-2.65 |
| | | yl]phenyl)methyl]piperazin- | | (m, 5H), 2.60-2.44 (m, 7H), 2.36- |
| | | 1-yl]propoxy)propyl]- | | 2.30 (m, 1H), 2.20-1.99 (m, 7H), |
| | | 1,3-benzodiazol-1- | | 1.96-1.85 (m, 2H), 1.78 (p, J = 6.3 |
| | | yl]piperidine-2,6-dione | | Hz, 2H), 1.69-1.66 (m, 1H), 1.58- |
| | | | | 1.46 (m, 2H), 1.41 (d, J = 6.8 Hz, |
| | | | | 3H), 1.05 (t, J = 7.3 Hz, 1H) |
| 167 | I-210 | 3-(3-methyl-2-oxo-5- | 915.45 | (400 MHz, CD₃OD) δ 8.76 (s, 1H), |
| | | [[(3R)-3-(3-[4-[(4-[7- | | 7.66-7.57 (m, 2H), 7.51-7.36 (m, |
| | | [trans-4- | | 3H), 7.22 (d, J = 1.5 Hz, 1H), 7.15- |
| | | hydroxycyclohexyl]-2- | | 7.05 (m, 2H), 5.34 (dd, J = 12.3, 5.4 |
| | | [[(2S)-4,4,4- | | Hz, 1H), 4.57 (s, 1H), 4.48 (q, J = |
| | | trifluorobutan-2- | | 6.5 Hz, 1H), 4.07 (d, J = 7.4 Hz, |
| | | yl]amino]pyrrolo[2,3- | | 1H), 3.81-3.63 (m, 3H), 3.58 (s, |
| | | d]pyrimidin-5- | | 2H), 3.49-3.39 (m, 7H), 3.01-2.85 |
| | | yl]phenyl)methyl]piperazin- | | (m, 2H), 2.82-2.68 (m, 5H), 2.64- |
| | | 1- | | 2.29 (m, 12H), 2.23-2.00 (m, 8H), |
| | | yl]propoxy)pyrrolidin-1- | | 1.86-1.79 (m, 1H), 1.75 (t, J = 7.6 |
| | | yl]methyl]-1,3- | | Hz, 2H), 1.54 (q, J = 7.7, 7.1 Hz, |
| | | benzodiazol-1- | | 2H), 1.42 (d, J = 6.7 Hz, 3H) |
| | | yl)piperidine-2,6-dione | | |
| 168 | I-209 | 3-(3-methyl-2-oxo-5- | 915.45 | (400 MHz, CD₃OD) δ 8.76 (s, 1H), |
| | | [[(3R)-3-(3-[4-[(4-[7- | | 7.62 (d, J = 7.8 Hz, 2H), 7.46-7.36 |
| | | [trans-4- | | (m, 3H), 7.21 (s, 1H), 7.10 (q, J = |
| | | hydroxycyclohexyl]-2- | | 8.1 Hz, 2H), 5.34 (dd, J = 12.6, 5.4 |
| | | [[(2R)-4,4,4- | | Hz, 1H), 4.56 (s, 1H), 4.48 (q, J = |
| | | trifluorobutan-2- | | 6.6 Hz, 1H), 4.05 (s, 1H), 3.75 (t, J = |
| | | yl]amino]pyrrolo[2,3- | | 9.0 Hz, 2H), 3.66 (d, J = 12.5 Hz, |
| | | d]pyrimidin-5- | | 1H), 3.58 (s, 2H), 3.50-3.41 (m, |
| | | yl]phenyl)methyl]piperazin- | | 7H), 2.96-2.86 (m, 2H), 2.83 (s, |
| | | 1- | | 1H), 2.81-2.67 (m, 5H), 2.66-2.48 |
| | | yl]propoxy)pyrrolidin-1- | | (m, 7H), 2.46 (t, J = 7.7 Hz, 2H), |
| | | yl]methyl-1,3- | | 2.38-2.34 (m, 2H), 2.19-1.99 (m, |
| | | benzodiazol-1- | | 8H), 1.81 (d, J = 10.4 Hz, 1H), 1.75 |
| | | yl)piperidine-2,6-dione | | (t, J = 7.6 Hz, 2H), 1.55 (d, J = 6.2 |
| | | | | Hz, 2H), 1.42 (d, J = 6.7 Hz, 3H) |
| 169 | I-198 | trans-3-(3-methyl-2-oxo- | 915.75 | (400 MHz, CD₃OD) δ 8.75 (s, 1H), |
| | | 5-[[(3S)-3-(3-[4-[(4-[4- | | 7.64-7.57 (m, 2H), 7.45-7.35 (m, |
| | | hydroxycyclohexyl]-2- | | 3H), 7.21 (d, J = 1.5 Hz, 1H), 7.14- |
| | | [[(2R)-4,4,4- | | 7.04 (m, 2H), 5.34 (m, 1H), 4.53 |
| | | trifluorobutan-2- | | (m, 2H), 4.05 (tt, J = 6.4, 3.1 Hz, |
| | | yl]amino]pyrrolo[2,3- | | 1H), 3.79-3.69 (m, 2H), 3.65 (m, |
| | | d]pyrimidin-5- | | 1H), 3.57 (s, 2H), 3.50-3.36 (m, |
| | | yl]phenyl)methyl]piperazin- | | 5H), 2.97-2.30 (m, 19H), 2.29-1.97 |
| | | 1- | | (m, 9H), 1.82-1.75 (m, 3H), 1.58- |
| | | yl]propoxy)pyrrolidin-1- | | 1.52 (m, 2H), 1.42 (d, J = 6.7 Hz, |
| | | yl]methyl]-1,3- | | 3H) |
| | | benzodiazol-1- | | |
| | | yl)piperidine-2,6-dione | | |
| 170 | I-199 | 3-(3-methyl-2-oxo-5- | 915.80 | (400 MHz, CD₃OD) δ 8.75 (s, 1H), |
| | | [[(3S)-3-(3-[4-[(4-[7- | | 7.61 (d, J = 8.1 Hz, 2H), 7.45-7.36 |
| | | [trans-4- | | (m, 3H), 7.21 (d, J = 1.5 Hz, 1H), |
| | | hydroxycyclohexyl]-2- | | 7.15-7.04 (m, 2H), 5.34 (m, 1H), |
| | | [[(2S)-4,4,4- | | 4.62-4.43 (m, 2H), 4.05 (dq, J = |
| | | trifluorobutan-2- | | 9.0, 3.3 Hz, 1H), 3.78-3.61 (m, |
| | | yl]amino]pyrrolo[2,3- | | 3H), 3.57 (s, 2H), 3.49-3.36 (m, |
| | | d]pyrimidin-5- | | 5H), 2.97-2.30 (m, 19H), 2.23-1.97 |
| | | yl]phenyl)methyl]piperazin- | | (m, 9H), 1.77 (m, 3H), 1.54 (m, |
| | | 1- | | 2H), 1.42 (d, J = 6.7 Hz, 3H) |
| | | yl]propoxy)pyrroidin-1- | | |
| | | yl]methyl]-1,3- | | |
| | | benzodiazol-1- | | |
| | | yl)piperidine-2,6-dione | | |
| 171 | I-194 | 3-[3-methyl-2-oxo-5-[2- | 872.50 | (400 MHz, CD₃OD) δ 8.75 (s, 1H), |
| | | (3-[4-[(4-[trans-4- | | 7.61 (d, J = 8.0 Hz, 2H), 7.45-7.35 |
| | | hydroxycyclohexyl]-2- | | (m, 3H), 7.08 (s, 1H), 7.06-6.97 (m, |
| | | [[(2R)-4,4,4- | | 2H), 5.32 (dd, J = 12.3, 5.3 Hz, |
| | | trifluorobutan-2- | | 1H), 4.61-4.44 (m, 2H), 3.81-3.69 |
| | | yl]amino]pyrrolo[2,3- | | (m, 2H), 3.66-3.57 (m, 4H), 3.43 |
| | | d]pyrimidin-5- | | (d, J = 2.0 Hz, 3H), 2.95-2.90 (m, |
| | | yl]phenyl)methyl] | | 3H), 2.86-2.65 (m, 4H), 2.54-2.51 |
| | | piperazin-1- | | (m, 3H), 2.51-2.29 (m, 7H), 2.21- |
| | | yl]cyclobutoxy)ethyl]- | | 2.01 (m, 8H), 1.80-1.66 (m, 2H), |
| | | 1,3-benzodiazol-1- | | 1.54 (s, 2H), 1.42 (d, J = 6.7 Hz, |
| | | yl]piperidine-2,6-dione | | |
| 172 | I-195 | 3-[3-methyl-2-oxo-5-[2- | 872.45 | (400 MHz, CD₃OD) δ 8.75 (s, 1H), |
| | | (3-[4-[(4-[7-[trans-4- | | 7.64-7.58 (m, 2H), 7.46-7.36 (m, |
| | | hydroxycyclohexyl]-2- | | 3H), 7.12-7.06 (m, 1H), 7.06-6.97 |
| | | [[(2S)-4,4,4- | | (m, 2H), 5.32 (dd, J = 12.3, 5.3 Hz, |
| | | trifluorobutan-2- | | 1H), 4.59-4.44 (m, 2H), 3.75 (td, J = |
| | | yl]amino]pyrrolo[2,3- | | 12.6, 11.0, 6.7 Hz, 2H), 3.66-3.54 |
| | | d]pyrimidin-5- | | (m, 4H), 3.44 (s, 3H), 2.92 (q, J = |
| | | yl]phenyl)methyl] | | 6.6, 6.0 Hz, 3H), 2.85-2.67 (m, |
| | | piperazin-1- | | 4H), 2.64-2.32 (m, 10H), 2.22-2.00 |
| | | yl]cyclobutoxy)ethyl]- | | (m, 8H), 1.75 (t, J = 9.2 Hz, 2H), |
| | | 1,3-benzodiazol-1- | | 1.60-1.50 (m, 2H), 1.42 (d, J = 6.7 |
| | | yl]piperidine-2,6-dione | | Hz, 3H) |
| 173 | I-200 | trans-3-(3-methyl-2-oxo- | 901.65 | (400 MHz, CD₃OD) δ 8.76 (s, 1H), |
| | | 5-[[3-(3-[4-[(4-[7-[4- | | 8.42 (s, 3H), 7.66 (d, J = 7.9 Hz, |
| | | hydroxycyclohexyl]-2- | | 2H), 7.49-7.41 (m, 3H), 7.29 (s, |
| | | [[(2R)-4,4,4- | | 1H), 7.20 (s, 2H), 5.38 (dd, J = |
| | | trifluorobutan-2- | | 12.4, 5.4 Hz, 1H), 4.62-4.42 (m, |
| | | yl]amino]pyrrolo[2,3- | | 2H), 4.35 (s, 1H), 4.28 (s, 1H), 4.18 |
| | | d]pyrimidin-5- | | (dd, J = 10.8, 6.1 Hz, 2H), 3.88- |
| | | yl]phenyl)methyl]piperazin- | | 3.70 (m, 5H), 3.53 (t, J = 5.8 Hz, |
| | | 1-yl]propoxy)azetidin- | | 2H), 3.47 (s, 3H), 3.07-3.01 (m, |
| | | 1-yl]methyl-1,3- | | 4H), 2.97-2.74 (m, 10H), 2.37 (m, |
| | | benzodiazol-1- | | 1H), 2.22-2.04 (m, 7H), 1.93 (t, J = |
| | | yl)piperidine-2,6-dione; | | 7.5 Hz, 2H), 1.57-1.52 (m, 2H), |
| | | tris(formic acid) | | 1.42 (d, J = 6.7 Hz, 3H), 1.31 (s, |
| | | | | 1H) |
| 174 | I-201 | 3-(3-methyl-2-oxo-5-[[3- | 901.75 | (400 MHz, CD₃OD) δ 8.76 (s, 1H), |
| | | (3-[4-[(4-[7-[trans-4- | | 8.38 (s, 3H), 7.66 (d, J = 7.9 Hz, |
| | | hydroxycyclohexyl]-2- | | 2H), 7.49-7.41 (m, 3H), 7.29 (s, |
| | | [[(2S)-4,4,4- | | 1H), 7.20 (s, 2H), 5.38 (dd, J = |
| | | trifluorobutan-2- | | 12.4, 5.3 Hz, 1H), 4.61-4.45 (m, |
| | | yl]amino]pyrrolo[2,3- | | 2H), 4.36 (s, 1H), 4.30 (s, 2H), |
| | | d]pyrimidin-5- | | 4.25-4.15 (m, 2H), 3.89-3.81 (m, |
| | | yl]phenyl)methyl]piperazin- | | 2H), 3.80 (s, 2H), 3.73 (m, 1H), |
| | | 1-yl]propoxy)azetidin- | | 3.53 (t, J = 5.8 Hz, 2H), 3.47 (s, |
| | | 1-yl]methyl]-1,3- | | 3H), 3.09-3.01 (m, 4H), 2.98-2.74 |
| | | benzodiazol-1- | | (m, 10H), 2.44-2.31 (m, 1H), 2.22- |
| | | yl)piperidine-2,6-dione; | | 2.04 (m, 7H), 1.95 (d, J = 7.9 Hz, |
| | | tris(formic acid) | | 2H), 1.59-1.49 (m, 2H), 1.42 (d, J = |
| | | | | 6.7 Hz, 3H), 1.31 (s, 1H) |
| 175 | I-205 | 3-(3-methyl-2-oxo-5-[2- | 838.45 | (400 MHz, CD₃OD) δ 8.43 (m, |
| | | [3-(4-[[trans-4-[2-[(2S)- | | 3H), 7.09 (d, J = 1.4 Hz, 1H), 7.06- |
| | | pentan-2-ylamino]-7- | | 6.99 (m, 2H), 6.89 (s, 1H), 5.33 |
| | | [trans-4- | | (dd, J = 12.3, 5.3 Hz, 1H), 4.50- |
| | | hydroxycyclohexyl] | | 4.38 (m, 1H), 4.10 (p, J = 6.5 Hz, |
| | | pyrrolo[2,3-d]pyrimidin-5- | | 1H), 3.82 (p, J = 7.1 Hz, 1H), 3.75- |
| | | yl]cyclohexyl]methyl] | | 3.61 (m, 3H), 3.44 (s, 3H), 3.10- |
| | | piperazin-1- | | 2.88 (m, 6H), 2.87-2.75 (m, 6H), |
| | | yl)cyclobutoxy]ethyl]- | | 2.68 (m, 3H), 2.58 (p, J = 6.8 Hz, |
| | | 1,3-benzodiazol-1- | | 2H), 2.36-2.03 (m, 5H), 1.97 (m, |
| | | yl)piperidine-2,6-dione; | | 5H), 1.89-1.73 (m, 3H), 1.71-1.41 |
| | | bis(formic acid) | | (m, 8H), 1.25-1.18 (m, 5H), 0.98 (t, |
| | | | | J = 7.1 Hz, 3H) |
| 176 | I-206 | 3-(3-methyl-2-oxo-5-[2- | 838.45 | (400 MHz, CD₃OD) δ 8.41 (s, 1H), |
| | | [3-(4-[[cis-4-[2-[(2S)- | | 7.15-6.98 (m, 3H), 6.84 (s, 1H), |
| | | pentan-2-ylamino]-7- | | 5.32 (dd, J = 12.4, 5.4 Hz, 1H), 4.43 |
| | | [trans-4- | | (p, J = 8.2 Hz, 1H), 4.09 (q, J = 6.5 |
| | | hydroxycyclohexyl] | | Hz, 1H), 3.77 (t, J = 6.9 Hz, 1H), |
| | | pyrrolo[2,3-d]pyrimidin-5- | | 3.70 (m, 1H), 3.62 (t, J = 6.8 Hz, |
| | | yl]cyclohexyl]methyl] | | 2H), 3.44 (s, 3H), 2.96-2.78 (m, |
| | | piperazin-1- | | 6H), 2.64-2.29 (m, 12H), 2.23-2.05 |
| | | yl)cyclobutoxy]ethyl]- | | (m, 4H), 2.03-1.91 (m, 5H), 1.84- |
| | | 1,3-benzodiazol-1- | | 1.59 (m, 11H), 1.53-1.42 (m, 5H), |
| | | yl)piperidine-2,6-dione | | 1.24 (d, J = 6.5 Hz, 3H), 0.98 (t, J = |
| | | | | 7.0 Hz, 3H) |
| 177 | I-207 | 3-[3-methyl-2-oxo-5-([4- | 895.90 | (400 MHz, CD₃OD) δ 8.41 (s, 1H), |
| | | [3-(4-[[cis-4-[2-[(2S)- | | 7.20 (s, 1H), 7.14-7.05 (m, 2H), |
| | | pentan-2-ylamino]-7- | | 6.84 (s, 1H), 5.35 (m, 1H), 4.44 (p, |
| | | [trans-4- | | J = 8.2 Hz, 1H), 4.08 (p, J = 6.5 Hz, |
| | | hydroxycyclohexyl] | | 1H), 3.72-3.63 (m, 1H), 3.61 (s, |
| | | pyrrolo[2,3-d]pyrimidin-5- | | 2H), 3.51 (t, J = 6.1 Hz, 2H), 3.45 |
| | | yl]cyclohexyl]methyl] | | (s, 3H), 3.39-3.35 (m, 1H), 2.96- |
| | | piperazin-1- | | 2.76 (m, 6H), 2.54-2.34 (m, 9H), |
| | | yl)propoxy]piperidin-1- | | 2.27 (s, 2H), 2.10-1.92 (m, 6H), |
| | | yl]methyl)-1,3- | | 1.79-1.70 (m, 7H), 1.71-1.59 (m, |
| | | benzodiazol-1- | | 13H), 1.58-1.38 (m, 6H), 1.31 (s, |
| | | yl]piperidine-2,6-dione | | 1H), 1.24 (d, J = 6.5 Hz, 3H), 0.98 |
| | | | | (t, J = 7.2 Hz, 3H) |
| 178 | I-208 | 3-[3-methyl-2-oxo-5-([4- | 895.90 | (400 MHz, CD₃OD) δ 8.42 (s, 1H), |
| | | [3-(4-[[trans-4-[2-[(2S)- | | 7.21 (s, 1H), 7.14-7.05 (m, 2H), |
| | | pentan-2-ylamino]-7- | | 6.81 (s, 1H), 5.35 (m, 1H), 4.47- |
| | | [trans-4- | | 4.39 (m, 1H), 4.09 (q, J = 6.4 Hz, |
| | | hydroxycyclohexyl] | | 1H), 3.72-3.64 (m, 1H), 3.62 (s, |
| | | pyrrolo[2,3-d]pyrimidin-5- | | 2H), 3.52 (d, J = 6.1 Hz, 2H), 3.45 |
| | | yl]cyclohexyl]methyl] | | (s, 3H), 2.98-2.88 (m, 1H), 2.85- |
| | | piperazin-1- | | 2.78 (m, 4H), 2.72-2.63 (m, 2H), |
| | | yl)propoxy]piperidin-1- | | 2.59-2.38 (m, 9H), 2.33-2.23 (m, |
| | | yl]methyl)-1,3- | | 4H), 2.23-2.17 (m, 1H), 2.12-2.04 |
| | | benzodiazol-1- | | (m, 4H), 2.03-1.86 (m, 8H), 1.78 (t, |
| | | yl]piperidine-2,6-dione | | J = 7.8 Hz, 2H), 1.69-1.57 (m, 5H), |
| | | | | 1.56-1.40 (m, 7H), 1.31 (s, 1H), |
| | | | | 1.24 (d, J = 6.5 Hz, 3H), 1.21-1.07 |
| | | | | (m, 4H), 0.98 (t, J = 7.1 Hz, 3H) |
| 179 | I-215 | 3-(5-[3-[3-(4-[[4-(2- | 842.35 | (400 MHz, DMSO-d₆) δ 11.08 (s, |
| | | [[(2R)-1,1- | | 1H), 8.88 (s, 1H), 7.68-7.60 (m, |
| | | difluoropropan-2- | | 3H), 7.31 (d, J = 7.9 Hz, 2H), 7.10- |
| | | yl]amino]-7-[trans-4- | | 6.97 (m, 3H), 6.87 (d, J = 8.3 Hz, |
| | | hydroxycyclohexyl] | | 1H), 6.07 (t, J = 56.9 Hz, 1H), 5.34 |
| | | pyrrolo[2,3-d]pyrimidin-5- | | (dd, J = 12.6, 5.5 Hz, 1H), 4.69 (d, |
| | | yl)phenyl]methyl]piperazin- | | J = 4.5 Hz, 1H), 4.44 (s, 2H), 3.55 |
| | | 1-yl)propoxy]propyl]- | | (s, 1H), 3.45 (s, 2H), 3.43-3.32 (m, |
| | | 3-methyl-2-oxo-1,3- | | 6H), 2.95-2.85 (m, 1H), 2.78-2.55 |
| | | benzodiazol-1- | | (m, 5H), 2.46-2.28 (m, 9H), 2.05- |
| | | yl)piperidine-2,6-dione | | 1.92 (m, 5H), 1.91 (s, 2H), 1.82 (q, |
| | | | | J = 7.5, 7.0 Hz, 2H), 1.65 (t, J = 7.0 |
| | | | | Hz, 2H), 1.37 (d, J = 12.2 Hz, 2H), |
| | | | | 1.24 (d, J = 6.7 Hz, 4H) |
| 180 | I-216 | 3-(5-[3-[3-(4-[[4-(2- | 842.35 | (400 MHz, DMSO-d₆) δ 11.08 (s, |
| | | [[(2S)-1,1- | | 1H), 8.88 (s, 1H), 7.71-7.60 (m, |
| | | difluoropropan-2- | | 3H), 7.31 (d, J = 7.9 Hz, 2H), 7.10- |
| | | yl]amino]-7-[trans-4- | | 6.97 (m, 3H), 6.87 (d, J = 8.1 Hz, |
| | | hydroxycyclohexyl] | | 1H), 6.07 (t, J = 56.3 Hz, 1H), 5.34 |
| | | pyrrolo[2,3-d]pyrimidin-5- | | (dd, J = 12.8, 5.5 Hz, 1H), 4.69 (d, |
| | | yl)phenyl]methyl]piperazin- | | J = 4.5 Hz, 1H), 4.43 (d, J = 14.0 |
| | | 1-yl)propoxy]propyl]- | | Hz, 2H), 3.55 (s, 1H), 3.45 (s, 2H), |
| | | 3-methyl-2-oxo-1,3- | | 3.43-3.33 (m, 6H), 2.97-2.83 (m, |
| | | benzodiazol-1- | | 1H), 2.77-2.57 (m, 5H), 2.46-2.24 |
| | | yl)piperidine-2,6-dione | | (m, 9H), 2.05-1.92 (m, 5H), 1.90 (s, |
| | | | | 2H), 1.81 (t, J = 7.4 Hz, 2H), 1.70- |
| | | | | 1.59 (m, 2H), 1.37 (d, J = 12.2 Hz, |
| | | | | 2H), 1.28-1.20 (m, 4H) |
| 181 | I-175 | 3-[3-methyl-2-oxo-5-[2- | 832.75 | (400 MHz, CD₃OD) δ 8.70 (s, 1H), |
| | | (3-[4-[(4-[2-[(2S)-pentan- | | 7.60 (dd, J = 8.3, 2.2 Hz, 2H), 7.42- |
| | | 2-ylamino]-7-[trans-4- | | 7.32 (m, 3H), 7.09 (d, J = 7.5 Hz, |
| | | hydroxycyclohexyl] | | 1H), 7.06-6.96 (m, 2H), 5.32 (dd, J = |
| | | pyrrolo[2,3-d]pyrimidin-5- | | 12.3, 5.3 Hz, 1H), 4.54 (p, J = 8.4, |
| | | yl]phenyl)methyl]piperazin- | | 8.0 Hz, 1H), 4.18-4.02 (m, 1H), |
| | | 1- | | 3.84-3.75 (m, 2H), 3.73-3.52 (m, |
| | | yl]cyclobutoxy)ethyl]- | | 4H), 3.43 (s, 3H), 2.95-2.77 (m, |
| | | 1,3-benzodiazol-1- | | 5H), 2.67-2.30 (m, 10H), 2.20-2.01 |
| | | yl]piperidine-2,6-dione | | (m, 8H), 1.79-1.62 (m, 3H), 1.57- |
| | | | | 1.41 (m, 5H), 1.27 (d, J = 6.5 Hz, |
| | | | | 3H), 1.00 (t, J = 7.1 Hz, 3H) |
| 182 | I-187 | 3-[3-methyl-2-oxo-5-[2- | 846.45 | (400 MHz, CD₃OD) δ 8.71 (s, 1H), |
| | | (3-[4-[(4-[2-[(2S)-pentan- | | 8.51 (s, 1H), 7.63 (d, J = 8.0 Hz, |
| | | 2-ylamino]-7-[cis-4- | | 2H), 7.46-7.37 (m, 3H), 7.08 (s, |
| | | hydroxy-4- | | 1H), 7.06-6.96 (m, 2H), 5.32 (dd, J = |
| | | methylcyclohexyl] | | 12.3, 5.3 Hz, 1H), 4.58 (s, 1H), |
| | | pyrrolo[2,3-d]pyrimidin-5- | | 4.15 (p, J = 6.5 Hz, 1H), 3.79 (p, J = |
| | | yl]phenyl)methyl] | | 7.1 Hz, 1H), 3.69 (s, 2H), 3.63 (t, |
| | | piperazin-1- | | J = 6.7 Hz, 2H), 3.43 (d, J = 1.9 Hz, |
| | | yl]cyclobutoxy)ethyl]- | | 3H), 2.98-2.86 (m, 3H), 2.85-2.79 |
| | | 1,3-benzodiazol-1- | | (m, 2H), 2.74-2.42 (m, 10H), 2.34- |
| | | yl]piperidine-2,6-dione; | | 2.13 (m, 4H), 1.91-1.75 (m, 6H), |
| | | formic acid | | 1.73-1.60 (m, 3H), 1.57-1.44 (m, |
| | | | | 3H), 1.40-1.27 (m, 3H), 1.26 (d, J = |
| | | | | 6.5 Hz, 3H), 0.99 (t, J = 7.2 Hz, |
| | | | | 3H) |
| 183 | I-188 | 3-[3-methyl-2-oxo-5-[2- | 846.55 | (400 MHz, CD₃OD) δ 8.71 (s, 1H), |
| | | (3-[4-[(4-[2-[(2S)-pentan- | | 8.51 (s, 1H), 7.68-7.61 (m, 2H), |
| | | 2-ylamino]-7-[trans-4- | | 7.47-7.39 (m, 3H), 7.12-6.97 (m, |
| | | hydroxy-4- | | 3H), 5.32 (dd, J = 12.3, 5.4 Hz, |
| | | methylcyclohexyl] | | 1H), 4.57-4.46 (m, 1H), 4.14 (q, J = |
| | | pyrrolo[2,3-d]pyrimidin-5- | | 6.6 Hz, 1H), 3.78 (d, J = 14.8 Hz, |
| | | yl]phenyl)methyl]piperazin- | | 3H), 3.64 (t, J = 6.7 Hz, 2H), 3.43 |
| | | 1- | | (s, 3H), 2.92 (q, J = 7.3, 6.5 Hz, |
| | | yl]cyclobutoxy)ethyl]- | | 4H), 2.89-2.62 (m, 10H), 2.55 (d, J = |
| | | 1,3-benzodiazol-1- | | 6.9 Hz, 2H), 2.32-2.08 (m, 4H), |
| | | yl]piperidine-2,6-dione; | | 1.99 (d, J = 12.9 Hz, 2H), 1.89-1.72 |
| | | formic acid | | (m, 6H), 1.68-1.43 (m, 7H), 1.27 |
| | | | | (d, J = 6.5 Hz, 3H), 0.99 (t, J = 7.2 |
| | | | | Hz, 3H) |
| 184 | I-185 | 3-(5-[3-[4-(4-[[4-(2- | 856.40 | (400 MHz, DMSO-d₆) δ 11.07 (s, |
| | | [[(2R)-1,1- | | 1H), 8.88 (s, 1H), 7.68-7.57 (m, |
| | | difluoropropan-2- | | 3H), 7.31 (d, J = 8.0 Hz, 2H), 7.13- |
| | | yl]amino]-7-[trans-4- | | 7.00 (m, 3H), 6.87 (dd, J = 8.1, 1.6 |
| | | hydroxycyclohexyl] | | Hz, 1H), 6.07 (t, J = 57.4 Hz, 1H), |
| | | pyrrolo[2,3-d]pyrimidin-5- | | 5.34 (dd, J = 12.6, 5.4 Hz, 1H), 4.69 |
| | | yl)phenyl]methyl]piperazin- | | (d, J = 4.5 Hz, 1H), 4.48-4.38 (m, |
| | | 1-yl)butoxy]propyl]-3- | | 2H), 3.54 (s, 1H), 3.45 (s, 2H), |
| | | methyl-2-oxo-1,3- | | 3.38-3.32 (m, 7H), 2.95-2.84 (m, |
| | | benzodiazol-1- | | 1H), 2.73-2.60 (m, 4h), 2.48-2.34 |
| | | yl)piperidine-2,6-dione | | (m, 6H), 2.34-2.15 (m, 3H), 2.03- |
| | | | | 1.88 (m, 7H), 1.81 (p, J = 6.5 Hz, |
| | | | | 2H), 1.52-1.33 (m, 6H), 1.24 (d, J = |
| | | | | 7.0 Hz, 3H) |
| 185 | I-186 | 3-(5-[3-[4-(4-[[4-(2- | 856.30 | (400 MHz, DMSO-d₆) δ 11.08 (s, |
| | | [[2S)-1,1- | | 1H), 8.88 (s, 1H), 7.68-7.58 (m, |
| | | difluoropropan-2- | | 3H), 7.31 (d, J = 7.9 Hz, 2H), 7.09- |
| | | yl]amino]-7-[trans-4- | | 6.97 (m, 3H), 6.86 (dd, J = 8.0, 1.6 |
| | | hydroxycyclohexyl] | | Hz, 1H), 6.07 (t, J = 57.4 Hz, 1H), |
| | | pyrrolo[2,3-d]pyrimidin-5- | | 5.34 (dd, J = 12.6, 5.4 Hz, 1H), 4.69 |
| | | yl)phenyl]methyl]piperazin- | | (d, J = 4.5 Hz, 1H), 4.45 (d, J = 12.5 |
| | | 1-yl)butoxy]propyl]-3- | | Hz, 2H), 3.55 (s, 1H), 3.45 (s, 2H), |
| | | methyl-2-oxo-1,3- | | 3.39-3.33 (m, 4H), 3.32 (s, 2H), |
| | | benzodiazol-1- | | 2.96-2.84 (m, 1H), 2.76-2.57 (m, |
| | | yl)piperidine-2,6-dione | | 4H), 2.48-2.18 (m, 9H), 2.10-1.69 |
| | | | | (m, 9H), 1.58-1.30 (m, 6H), 1.24 |
| | | | | (d, J = 6.8 Hz, 3H) |
| 186 | I-192 | 3-(5-[3-[3-(4-[[4-(2- | 856.40 | (400 MHz, CD₃OD) δ 8.75 (s, 1H), |
| | | [[(2R)-4,4-difluorobutan- | | 7.61 (d, J = 8.0 Hz, 2H), 7.44-7.38 |
| | | 2-yl]amino]-7-[trans-4- | | (m, 3H), 7.05-6.96 (m, 3H), 6.24- |
| | | hydroxycyclohexyl] | | 5.87 (m, 1H), 5.32 (dd, J = 12.4, 5.6 |
| | | pyrrolo[2,3-d]pyrimidin-5- | | Hz, 1H), 4.63-4.36 (m, 2H), 3.73 |
| | | yl)phenyl]methyl]piperazin- | | (d, J = 11.4 Hz, 1H), 3.59 (s, 2H), |
| | | 1-yl)propoxy]propyl]- | | 3.51-3.41 (m, 7H), 2.95-2.74 (m, |
| | | 3-methyl-2-oxo-1,3- | | 5H), 2.66-2.45 (m, 8H), 2.25-2.00 |
| | | benzodiazol-1- | | (m, 9H), 1.95-1.87 (m, 2H), 1.79 (s, |
| | | yl)piperidine-2,6-dione | | 2H), 1.55 (t, J = 8.8 Hz, 2H), 1.40- |
| | | | | 1.29 (m, 6H) |
| 187 | I-193 | 3-(5-[3-[3-(4-[[4-(2- | 856.55 | (400 MHz, CD₃OD) δ 8.75 (s, 1H), |
| | | [[(2S)-4,4-difluorobutan- | | 7.61 (d, J = 8.0 Hz, 2H), 7.46-7.37 |
| | | 2-yl]amino]-7-[trans-4- | | (m, 3H), 7.07-6.95 (m, 3H), 6.22- |
| | | hydroxycyclohexyl] | | 5.81 (m, 1H), 5.32 (dd, J = 12.4, 5.4 |
| | | pyrrolo[2,3-d]pyrimidin-5- | | Hz, 1H), 4.61-4.35 (m, 2H), 3.79- |
| | | yl)phenyl]methyl]piperazin- | | 3.68 (m, 1H), 3.58 (s, 2H), 3.53- |
| | | 1-yl)propoxy]propyl]- | | 3.42 (m, 7H), 2.96-2.75 (m, 5H), |
| | | 3-methyl-2-oxo-1,3- | | 2.61-2.44 (m, 8H), 2.19-1.92 (m, |
| | | benzodiazol-1- | | 9H), 1.91-1.79 (m, 4H), 1.61-1.48 |
| | | yl)piperidine-2,6-dione | | (m, 2H), 1.37-1.30 (m, 5H) |
| 188 | I-176 | 3-(3-methyl-2-oxo-5- | 875.80 | (400 MHz, CD₃OD) δ 8.71 (s, 1H), |
| | | [[(3R)-3-(3-[4-[(4-[2- | | 7.60 (d, J = 7.8 Hz, 2H), 7.42-7.36 |
| | | [(2S)-pentan-2-ylamino]- | | (m, 3H), 7.21 (d, J = 1.4 Hz, 1H), |
| | | 7-[(trans-4- | | 7.10 (q, J = 8.2 Hz, 2H), 5.34 (m, |
| | | hydroxycyclohexyl] | | 1H), 4.58-4.50 (m, 1H), 4.16-4.03 |
| | | pyrrolo[2,3-d]pyrimidin-5- | | (m, 2H), 3.72 (m, 3H), 3.57 (s, 2H), |
| | | yl]phenyl)methyl]piperazin- | | 3.55-3.34 (m, 5H), 2.98-2.74 (m, |
| | | 1- | | 5H), 2.66-2.42 (m, 10H), 2.22-2.00 |
| | | yl]propoxy)pyrrolidin-1- | | (m, 9H), 1.74 (m, 4H), 1.57-1.44 |
| | | yl]methyl]-1,3- | | (m, 5H), 1.33-1.24 (m, 5H), 1.00 (t, |
| | | benzodiazol-1- | | J = 7.0 Hz, 3H), 0.91 (d, J = 8.3 Hz, |
| | | yl)piperidine-2,6-dione | | 1H) |
| 189 | I-177 | 3-(3-methyl-2-oxo-5- | 875.80 | (400 MHz, CD₃OD) δ 8.72 (s, 1H), |
| | | [[(3S)-3-(3-[4-[(4-[2- | | 7.64-7.57 (m, 2H), 7.51-7.31 (m, |
| | | [(2S)-pentan-2-ylamino]- | | 4H), 7.21 (d, J = 1.5 Hz, 1H), 7.15- |
| | | 7-[trans-4- | | 7.05 (m, 2H), 5.34 (dd, J = 12.4, 5.5 |
| | | hydroxycyclohexyl] | | Hz, 1H), 4.54 (p, J = 8.3, 7.8 Hz, |
| | | pyrrolo[2,3-d]pyrimidin-5- | | 1H), 4.13 (q, J = 6.4 Hz, 1H), 4.06 |
| | | yl]phenyl)methyl]piperazin- | | (dt, J = 6.7, 3.4 Hz, 1H), 3.80-3.63 |
| | | 1- | | (m, 3H), 3.57 (s, 2H), 3.49-3.39 (m, |
| | | yl]propoxy)pyrrolidin-1- | | 5H), 2.95-2.73 (m, 5H), 2.66-2.38 |
| | | yl]methyl]-1,3- | | (m, 11H), 2.22-2.01 (m, 8H), 1.86- |
| | | benzodiazol-1- | | 1.64 (m, 4H), 1.61-1.44 (m, 5H), |
| | | yl)piperidine-2,6-dione | | 1.29 (m, 5H), 1.00 (t, J = 7.1 Hz, |
| | | | | 3H) |
| 190 | I-189 | 3-(3-methyl-2-oxo-5-[[4- | 889.55 | (400 MHz, CD₃OD) δ 8.71 (s, 1H), |
| | | (3-[4-[(4-[2-[(2S)-pentan- | | 8.36 (s, 1H), 7.65 (d, J = 8.1 Hz, |
| | | 2-ylamino]-7-[trans-4- | | 2H), 7.47-7.41 (m, 3H), 7.33 (s, |
| | | hydroxycyclohexyl] | | 1H), 7.28-7.18 (m, 2H), 5.38 (m, |
| | | pyrrolo[2,3-d]pyrimidin-5- | | 1H), 4.59-4.51 (m, 1H), 4.27 (s, |
| | | yl]phenyl)methyl]piperazin- | | 2H), 4.14 (q, J = 6.5 Hz, 1H), 3.82- |
| | | 1- | | 3.72 (m, 3H), 3.69-3.64 (m, 1H), |
| | | yl]propoxy)piperidin-1- | | 3.62-3.56 (m, 2H), 3.48 (s, 3H), |
| | | yl]methyl]-1,3- | | 3.26 (s, 2H), 3.18-3.02 (m, 4H), |
| | | benzodiazol-1- | | 2.96 (d, J = 7.7 Hz, 3H), 2.92-2.76 |
| | | yl)piperidine-2,6-dione; | | (m, 6H), 2.25-2.04 (m, 9H), 1.97- |
| | | formic acid | | 1.89 (m, 4H), 1.75-1.66 (m, 1H), |
| | | | | 1.57-1.44 (m, 5H), 1.27 (d, J = 6.5 |
| | | | | Hz, 3H), 1.00 (t, J = 7.1 Hz, 3H) |
| 191 | I-178 | 3-(3-methyl-2-oxo-5-[[3- | 861.75 | (400 MHz, CD₃OD) δ 8.71 (s, 1H), |
| | | (3-[4-[(4-[2-[(2S)-pentan- | | 7.61 (d, J = 7.8 Hz, 2H), 7.39 (d, J = |
| | | 2-ylamino]-7-[trans-4- | | 7.2 Hz, 3H), 7.15 (s, 1H), 7.08 (s, |
| | | hydroxycyclohexyl] | | 2H), 5.34 (dd, J = 12.5, 5.4 Hz, |
| | | pyrrolo[2,3-d]pyrimidin-5- | | 1H), 4.59-4.50 (m, 1H), 4.17-4.09 |
| | | yl]phenyl)methyl]piperazin- | | (m, 2H), 3.72 (s, 3H), 3.68-3.52 (m, |
| | | 1-yl]propoxy)azetidin- | | 4H), 3.50-3.34 (m, 5H), 3.08 (t, J = |
| | | 1-yl]methyl]-1,3- | | 7.0 Hz, 2H), 2.96-2.78 (m, 3H), |
| | | benzodiazol-1- | | 2.67-2.43 (m, 9H), 2.22-2.00 (m, |
| | | yl)piperidine-2,6-dione | | 8H), 1.80-1.65 (m, 3H), 1.60-1.40 |
| | | | | (m, 6H), 1.34-1.24 (m, 4H), 1.00 (t, |
| | | | | J = 7.0 Hz, 3H) |
| 151-1 | 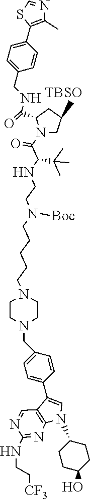 [SMILES: Cc1ncsc1-c1ccc(CNC(=O)[C@@H]2C[C@@H](O[Si](C)(C)C(C)(C)C)CN2C(=O)[C@@H](NCCN(CCCCCN2CCN(Cc3ccc(-c4cn([C@H]5CC[C@H](O)CC5)c5nc(NCCC(F)(F)F)ncc45)cc3)CC2)C(=O)OC(C)(C)C)C(C)(C)C)cc1] | tert-butyl N-[2-[(1-[4- [(tert- butyldimethylsilyl)oxy]- 2-([[4-(4-methyl-1,3- thiazol-5- yl)phenyl]methyl]carbamoyl) pyrrolidin-1-yl]-3,3- dimethyl-1-oxobutan-2- yl)amino]ethyl]-N-[5-[4- ([4-[7-(4- hydroxycyclohexyl)-2- [(3,3,3- trifluoropropyl)amino]- 7H-pyrrolo[2,3- d]pyrimidin-5- yl]phenyl]methyl) piperazin-1-yl]pentyl] carbamate | 1259.1 | (400 MHz, CD₃OD) δ 8.86 (d, J = 7.0 Hz, 1H), 8.73 (s, 1H), 7.60 (d, J = 8.1 Hz, 2H), 7.48-7.30 (m, 7H), 4.74-4.46 (m, 7H), 4.35 (d, J = 15.5 Hz, 1H), 3.75-3.70 (m, 5H), 3.60 (s, 3H), 3.18 (d, J = 7.9 Hz, 2H), 2.66 (s, 2H), 2.45 (s, 3H), 2.22-2.00 (m, 9H), 1.91 (s, 4H), 1.62-1.50 (m, 6H), 1.43 (s, 9H), 1.35-1.28 (m, 4H), 0.99 (d, J = 6.1 Hz, 9H), 0.87 (d, J = 6.4 Hz, 9H), 0.72 (s, 1H), 0.50 (s, 3H), 0.28 (s, 1H), 0.11 (s, 3H), 0.09-0.06 (m, 2H) |
| 192 | I-45 | 3-[5-[3-(2-[2-[([4-[2- | 781.50 | (400 MHz, DMSO-d₆) δ 8.79 (s, |
| | | (butylamino)-7-[trans-4- | | 1H), 8.25 (s, 1H), 7.66 (d, J = 7.9 |
| | | hydroxycyclohexyl]-7H- | | Hz, 2H), 7.59 (s, 1H), 7.42 (d, J = |
| | | pyrrolo[2,3-d]pyrimidin- | | 7.9 Hz, 2H), 6.98 (d, J = 9.9 Hz, |
| | | 5- | | 2H), 6.85 (d, J = 8.2 Hz, 1H), 5.29 |
| | | yl]phenyl]methyl)amino] | | (dd, J = 12.7, 5.4 Hz, 1H), 4.39 (d, |
| | | ethoxy]ethoxy)propyl]-3- | | J = 11.9 Hz, 1H), 3.95 (s, 2H), 3.70 |
| | | methyl-2-oxo-2,3- | | (s, 3H), 3.58-3.43 (m, 5H), 3.39 (t, |
| | | dihydro-1H-1,3- | | J = 6.5 Hz, 2H), 3.37-3.21 (m, 5H), |
| | | benzodiazol-1- | | 2.95-2.81 (m, 3H), 2.73-2.61 (m, |
| | | yl]piperidine-2,6-dione; | | 4H), 2.12-1.89 (m, 4H), 1.88 (s, |
| | | formic acid | | 2H), 1.80 (q, J = 7.1 Hz, 2H), 1.54 |
| | | | | (p, J = 7.2 Hz, 2H), 1.45-1.28 (m, |
| | | | | 4H), 0.91 (t, J = 7.3 Hz, 3H) |
| 193 | I-54 | 3-[5-(3-[2-[([4-[2- | 737.50 | (400 MHz, CD₃OD) δ 8.71 (s, 1H), |
| | | (butylamino)-7-[trans-4- | | 7.74-7.68 (m, 2H), 7.53-7.45 (m, |
| | | hydroxycyclohexyl]-7H- | | 3H), 7.06-6.94 (m, 3H), 5.32 (m, |
| | | pyrrolo[2,3-d]pyrimidin- | | 1H), 4.56 (m, 1H), 4.13 (s, 2H), |
| | | 5- | | 3.79-2.63 (m, 3H), 3.54 (t, J = 6.4 |
| | | yl]phenyl]methyl)amino] | | Hz, 2H), 3.46 (t, J = 7.1 Hz, 2H), |
| | | ethoxy]propyl)-3-methyl- | | 3.39 (s, 3H), 3.14 (t, J = 5.1 Hz, |
| | | 2-oxo-2,3-dihydro-1H- | | 2H), 2.96-2.88 (m, 1H), 2.85-2.71 |
| | | 1,3-benzodiazol-1- | | (m, 4H), 2.25-2.09 (m, 3H), 2.08- |
| | | yl]piperidine-2,6-dione | | 2.01 (m, 4H), 2.00-1.92 (m, 2H), |
| | | | | 1.78-1.62 (m, 2H), 1.60-1.43 (m, |
| | | | | 4H), 1.02 (t, J = 7.4 Hz, 3H) |
| 194 | I-46 | 3-(5-[3-[([4-[2- | 693.50 | (400 MHz, DMSO-d₆) δ 11.11 (s, |
| | | (butylamino)-7-[trans-4- | | 1H), 8.84 (s, 1H), 8.28 (s, 1H), |
| | | hydroxycyclohexyl]-7H- | | 7.80-7.51 (m, 3H), 7.40 (d, J = 7.9 |
| | | pyrrolo[2,3-d]pyrimidin- | | Hz, 2H), 7.17-6.94 (m, 2H), 6.94- |
| | | 5- | | 6.70 (m, 2H), 5.34 (dd, J = 12.7, |
| | | yl]phenyl]methyl)amino] | | 5.4 Hz, 1H), 4.51-4.33 (m, 1H), |
| | | propyl]-3-methyl-2-oxo- | | 3.85 (s, 2H), 3.58-3.48 (m, 1H), |
| | | 2,3-dihydro-1H-1,3- | | 3.37-3.32 (m, 5H), 2.97-2.83 (m, |
| | | benzodiazol-1- | | 1H), 2.77-2.58 (m, 6H), 2.51 (p, J = |
| | | yl)piperidine-2,6-dione; | | 1.8 Hz, 2H), 2.07-1.91 (m, 4H), |
| | | formic acid | | 1.92-1.74 (m, 5H), 1.56 (p, J = 7.2 |
| | | | | Hz, 2H), 1.47-1.31 (m, 4H), 0.93 |
| | | | | (t, J = 7.4 Hz, 3H) |
| 195 | I-61 | 3-(5-[15-[4-([4-[2- | 939.50 | (400 MHz, CD₃OD) δ 8.72 (s, 1H), |
| | | (butylamino)-7-[trans-4- | | 8.46 (s, 2H), 7.63 (d, J = 7.7 Hz, |
| | | hydroxycyclohexyl]-7H- | | 2H), 7.46 (s, 1H), 7.39 (d, J = 7.8 |
| | | pyrrolo[2,3-d]pyrimidin- | | Hz, 2H), 6.87 (d, J = 8.4 Hz, 1H), |
| | | 5- | | 6.54 (s, 1H), 6.48 (d, J = 8.4 Hz, |
| | | yl]phenyl]methyl)piperazin- | | 1H), 5.24 (dd, J = 12.6, 5.4 Hz, |
| | | 1-yl]-4,7,10,13- | | 1H), 4.55 (p, J = 8.1 Hz, 1H), 3.78- |
| | | tetraoxa-1- | | 3.55 (m, 18H), 3.46 (t, J = 7.1 Hz, |
| | | azapentadecan-1-yl]-3- | | 3H), 3.35-3.32 (m, 4H), 3.26 (t, J = |
| | | methyl-2-oxo-2,3- | | 5.2 Hz, 2H), 3.22-3.20 (m, 5H), |
| | | dihydro-1H-1,3- | | 3.00-2.69 (m, 6H), 2.23-1.94 (m, |
| | | benzodiazol-1- | | 7H), 1.67 (p, J = 7.2 Hz, 2H), 1.59- |
| | | yl)piperidine-2,6-dione; | | 1.42 (m, 4H), 1.02 (t, J = 7.3 Hz, |
| | | bis(formic acid) | | 3H) |
| 196 | I-53 | 3-[5-(4-[4-[([4-[2- | 799.50 | (400 MHz, CD₃OD) δ 8.82 (s, 1H), |
| | | (butylamino)-7-[trans-4- | | 7.94 (s, 1H), 7.77 (d, J = 7.8 Hz, |
| | | hydroxycyclohexyl]-7H- | | 2H), 7.58 (d, J = 7.8 Hz, 2H), 7.05- |
| | | pyrrolo[2,3-d]pyrimidin- | | 6.96 (m, 2H), 6.93 (d, J = 8.0 Hz, |
| | | 5- | | 1H), 5.39-5.26 (m, 1H), 4.64-4.57 |
| | | yl]phenyl]methyl)amino] | | (m, 1H), 4.19 (s, 2H), 3.75-3.70 |
| | | butoxy]butyl)-3-methyl- | | (m, 1H), 3.56 (t, J = 7.2 Hz, 2H), |
| | | 2-oxo-2,3-dihydro-1H- | | 3.52-3.45 (m, 4H), 3.37 (s, 3H), |
| | | 1,3-benzodiazol-1- | | 3.11 (t, J = 7.9 Hz, 2H), 2.97-2.89 |
| | | yl]piperidine-2,6-dione | | (m, 1H), 2.84-2.75 (m, 2H), 2.66 |
| | | hydrochloride | | (t, J = 7.6 Hz, 2H), 2.19-2.11 (m, |
| | | | | 3H), 2.17-2.01 (m, 4H), 1.84 (t, J = |
| | | | | 7.8 Hz, 2H), 1.79-1.62 (m, 6H), |
| | | | | 1.61-1.49 (m, 6H), 1.31 (s, 1H), |
| | | | | 1.05 (t, J = 7.4 Hz, 3H) |
| 197 | I-66 | 3-[5-[3-(3-[[1-([4-[2- | 835.35 | (400 MHz, DMSO-d₆) δ 11.11 (s, |
| | | (butylamino)-7-[trans-4- | | 1H), 8.82 (s, 1H), 7.68-7.51 (m, |
| | | hydroxycyclohexyl]-7H- | | 3H), 7.30 (d, J = 7.7 Hz, 2H), 7.09- |
| | | pyrrolo[2,3-d]pyrimidin- | | 6.95 (m, 2H), 6.86 (d, J = 8.2 Hz, |
| | | 5- | | 2H), 5.34 (dd, J = 12.8, 5.4 Hz, |
| | | yl]phenyl]methyl) | | 1H), 4.73 (d, J = 4.5 Hz, 1H), 4.44 |
| | | piperidin-4- | | (d, J = 12.1 Hz, 1H), 3.67-3.42 (m, |
| | | yl]oxy]propoxy)propyl]- | | 8H), 3.30-3.12 (m, 6H), 2.97-2.56 |
| | | 3-methyl-2-oxo-2,3- | | (m, 7H), 2.25-2.04 (m, 2H), 2.04- |
| | | dihydro-1H-1,3- | | 1.92 (m, 5H), 1.91-1.82 (m, 3H), |
| | | benzodiazol-1- | | 1.81-1.66 (m, 4H), 1.61-1.57 (m, |
| | | yl]piperidine-2,6-dione | | 2H), 1.56 (p, J = 7.1 Hz, 2H), 1.38 |
| | | | | (m, 6H), 0.93 (t, J = 7.3 Hz, 3H) |
| 198 | I-67 | 4-[([4-[2-(butylamino)-7- | 836.20 | (400 MHz, CD₃OD) δ 8.72 (d, J = |
| | | [trans-4- | | 4.2 Hz, 1H), 8.48 (s, 1H), 7.72 (t, J = |
| | | hydroxycyclohexyl]-7H- | | 7.8 Hz, 2H), 7.54-7.45 (m, 3H), |
| | | pyrrolo[2,3-d]pyrimidin- | | 7.03-6.96 (m, 2H), 6.93 (dd, J = |
| | | 5- | | 8.7, 7.2 Hz, 1H), 5.31 (dd, J = 12.7, |
| | | yl]phenyl]methyl)amino]- | | 4.9 Hz, 1H), 4.60-4.49 (m, 1H), |
| | | N-(2-[3-[1-(2,6- | | 4.17 (d, J = 11.8 Hz, 2H), 3.73 (s, |
| | | dioxopiperidin-3-yl)-3- | | 1H), 3.69-3.51 (m, 4H), 3.50-3.44 |
| | | methyl-2-oxo-2,3- | | (m, 4H), 3.35-3.31 (s, 3H), 3.19- |
| | | dihydro-1H-1,3- | | 3.02 (m, 5H), 2.95-2.86 (m, 1H), |
| | | benzodiazol-5- | | 2.84-2.66 (m, 5H), 2.60 (t, J = 6.5 |
| | | yl]propoxy]ethyl)-N- | | Hz, 1H), 2.19-2.06 (m, 3H), 2.03 |
| | | methylbutanamide; | | (m, 6H), 1.88 (t, J = 6.8 Hz, 2H), |
| | | formic acid | | 1.68 (p, J = 7.2 Hz, 2H), 1.61-1.42 |
| | | | | (m, 4H), 1.02 (td, J = 7.4, 1.4 Hz, |
| | | | | 3H) |
| 199 | I-47 | 3-[3-methyl-2-oxo-5-[1- | 865.40 | (400 MHz, CD₃OD) δ 8.75 (s, 1H), |
| | | (4-[7-[trans-4- | | 7.73 (d, J = 8.0 Hz, 2H), 7.57-7.45 |
| | | hydroxycyclohexyl]-2- | | (m, 3H), 6.98 (dd, J = 4.7, 3.3 Hz, |
| | | [(3,3,3- | | 2H), 6.92 (dd, J = 8.0, 1.4 Hz, 1H), |
| | | trifluoropropyl)amino]- | | 5.30 (dd, J = 12.8, 5.5 Hz, 1H), |
| | | 7H-pyrrolo[2,3- | | 4.69-4.46 (m, 1H), 4.25 (s, 2H), |
| | | d]pyrimidin-5- | | 3.79 (t, J = 4.9 Hz, 2H), 3.77-3.65 |
| | | yl]phenyl)-5,8,11-trioxa- | | (m, 9H), 3.60 (dd, J = 5.7, 3.1 Hz, |
| | | 2-azatetradecan-14-yl]- | | 2H), 3.46 (t, J = 6.4 Hz, 2H), 3.36 |
| | | 2,3-dihydro-1H-1,3- | | (s, 3H), 3.27 (t, J = 5.0 Hz, 2H), |
| | | benzodiazol-1- | | 2.99-2.86 (m, 1H), 2.83-2.67 (m, |
| | | yl]piperidine-2,6-dione | | 4H), 2.58 (ddt, J = 14.2, 11.2, 5.5 |
| | | | | Hz, 2H), 2.18-2.10 (m, 3H), 2.17- |
| | | | | 2.01 (m, 4H), 1.93-1.81 (m, 2H), |
| | | | | 1.55 (q, J = 9.8, 8.8 Hz, 2H) |
| 200 | I-51 | 3-(3-methyl-2-oxo-5-[3- | 849.40 | (400 MHz, CD₃OD) δ 8.91 (d, J = |
| | | [3-(3-[[(4-[7-[trans-4- | | 4.7 Hz, 1H), 8.01 (d, J = 8.1 Hz, |
| | | hydroxycyclohexyl]-2- | | 1H), 7.81 (dd, J = 8.3, 2.2 Hz, 2H), |
| | | [(3,3,3- | | 7.62 (d, J = 8.0 Hz, 2H), 7.03 (d, J = |
| | | trifluoropropyl)amino]- | | 8.0 Hz, 1H), 7.00-6.91 (m, 2H), |
| | | 7H-pyrrolo[2,3- | | 5.39-5.25 (m, 1H), 4.66-4.56 (m, |
| | | d]pyrimidin-5- | | 1H), 4.25 (s, 2H), 3.86 (t, J = 7.0 |
| | | yl]phenyl)methyl]amino] | | Hz, 2H), 3.75-3.68 (m, 1H), 3.67- |
| | | propoxy)propoxy]propyl]- | | 3.52 (m, 4H), 3.49 (t, J = 6.3 Hz, |
| | | 2,3-dihydro-1H-1,3- | | 2H), 3.49-3.34 (m, 5H), 3.21 (t, J = |
| | | benzodiazol-1- | | 7.3 Hz, 2H), 3.04-2.94 (m, 1H), |
| | | yl)piperidine-2,6-dione | | 2.88-2.76 (m, 2H), 2.75-2.61 (m, |
| | | hydrochloride | | 4H), 2.23-1.95 (m, 9H), 1.92-1.79 |
| | | | | (m, 4H), 1.55 (m, 2H) |
| 201 | I-52 | 3-(3-methyl-2-oxo-4-[3- | [(M/2 + | (400 MHz, CD₃OD) δ 8.89 (s, 1H), |
| | | [3-(3-[[(4-[7-[trans-4- | 1)]⁺ = | 8.00 (s, 1H), 7.84-7.77 (m, 2H), |
| | | hydroxycyclohexyl]-2- | 425.50 | 7.68-7.61 (m, 2H), 7.07-6.96 (m, |
| | | [(3,3,3- | | 2H), 6.89 (dd, J = 6.8, 2.1 Hz, 1H), |
| | | trifluoropropyl)amino]- | | 5.39-5.24 (m, 1H), 4.62 (p, J = 8.6 |
| | | 7H-pyrrolo[2,3- | | Hz, 1H), 4.29 (s, 2H), 3.86 (t, J = |
| | | d]pyrimidin-5- | | 7.0 Hz, 2H), 3.77-3.68 (m, 1H), |
| | | yl]phenyl)methyl]amino] | | 3.63-3.53 (m, 7H), 3.49-3.34 (m, |
| | | propoxy)propoxy]propyl]- | | 4H), 3.22 (t, J = 7.3 Hz, 2H), 3.04- |
| | | 2,3-dihydro-1H-1,3- | | 2.90 (m, 3H), 2.89-2.77 (m, 2H), |
| | | benzodiazol-1- | | 2.73-2.59 (m, 2H), 2.21-2.11 (m, |
| | | yl)piperidine-2,6-dione | | 3H), 2.11-1.96 (m, 6H), 1.90-1.79 |
| | | hydrochloride | | (m, 4H), 1.54 (m, 2H) |
| 202 | I-48 | 3-(3-methyl-2-oxo-5-[3- | 821.40 | (400 MHz, DMSO-d₆) δ 11.10 (s, |
| | | [2-(2-[[(4-[7-[trans-4- | | 1H), 8.87 (s, 1H), 8.20 (s, 1H), |
| | | hydroxycyclohexyl]-2- | | 7.65 (d, J = 7.8 Hz, 3H), 7.39 (d, J = |
| | | [(3,3,3- | | 7.9 Hz, 2H), 7.26-6.94 (m, 3H), |
| | | trifluoropropyl)amino]- | | 6.86 (dd, J = 8.0, 1.6 Hz, 1H), 5.33 |
| | | 7H-pyrrolo[2,3- | | (dd, J = 12.7, 5.4 Hz, 1H), 4.51- |
| | | d]pyrimidin-5- | | 4.32 (m, 1H), 3.84 (s, 2H), 3.74- |
| | | yl]phenyl)methyl]amino] | | 3.46 (m, 10H), 3.40 (t, J = 6.5 Hz, |
| | | ethoxy)ethoxy]propyl]- | | 2H), 3.30 (s, 3H), 2.89 (t, J = 14.8 |
| | | 2,3-dihydro-1H-1,3- | | Hz, 1H), 2.79 (s, 2H), 2.72-2.58 |
| | | benzodiazol-1- | | (m, 6H), 2.05-1.86 (m, 7H), 1.81 |
| | | yl)piperidine-2,6-dione | | (p, J = 6.6 Hz, 2H), 1.38 (t, J = 12.1 |
| | | | | Hz, 2H) |
| 203 | I-99 | 3-[6-[11-(4-[7-[trans-4- | 824.35 | (400 MHz, CD₃OD) δ 8.86 (s, 1H), |
| | | hydroxycyclohexyl]-2- | | 8.59-8.48 (m, 2H), 8.39 (d, J = 2.2 |
| | | [(3,3,3- | | Hz, 1H), 7.96 (s, 1H), 7.81 (d, J = |
| | | trifluoropropyl)amino]- | | 8.9 Hz, 1H), 7.74 (d, J = 8.0 Hz, |
| | | 7H-pyrrolo[2,3- | | 2H), 7.69 (dd, J = 8.8, 2.2 Hz, 1H), |
| | | d]pyrimidin-5- | | 7.64 (d, J = 8.0 Hz, 2H), 7.33 (dd, |
| | | yl]phenyl)-4,7-dioxa- | | J = 7.7, 4.9 Hz, 1H), 6.11 (d, J = |
| | | 1,10-diazaundecan-1-yl]- | | 11.9 Hz, 1H), 4.66-4.58 (m, 1H), |
| | | 9H-pyrido[2,3-b]indol-9- | | 4.30 (s, 2H), 3.91-3.78 (m, 10H), |
| | | yl]piperidine-2,6-dione; | | 3.73 (t, J = 5.1 Hz, 3H), 3.36-3.25 |
| | | trihydrochloride | | (m, 4H), 3.13-3.04 (m, 1H), 2.94- |
| | | | | 2.85 (m, 1H), 2.74-2.61 (m, 2H), |
| | | | | 2.32-2.22 (m, 1H), 2.19-2.01 (m, |
| | | | | 6H), 1.55 (p, J = 10.8 Hz, 2H) |
| 204 | I-141 | 3-[3-methyl-2-oxo-5-[3- | 874.45 | (400 MHz, CD₃OD) δ 8.77 (s, 1H), |
| | | (3-[4-[(4-[7-[cis-4- | | 7.62 (d, J = 7.9 Hz, 2H), 7.46 (s, |
| | | hydroxy-4- | | 1H), 7.40 (d, J = 7.9 Hz, 2H), 7.05- |
| | | methylcyclohexyl]-2- | | 6.93 (m, 3H), 5.32 (dd, J = 12.3, |
| | | [(3,3,3- | | 5.4 Hz, 1H), 4.61 (d, J = 12.0 Hz, |
| | | trifluoropropyl)amino] | | 1H), 3.73 (t, J = 7.1 Hz, 2H), 3.59 |
| | | pyrrolo[2,3-d]pyrimidin-5- | | (s, 2H), 3.54-3.35 (m, 7H), 3.20- |
| | | yl]phenyl)methyl]piperazin- | | 2.71 (m, 6H), 2.70-2.34 (m, 11H), |
| | | 1-yl]propoxy)propyl]- | | 2.34-2.15 (m, 3H), 1.98-1.74 (m, |
| | | 1,3-benzodiazol-1- | | 8H), 1.67 (t, J = 12.6 Hz, 2H), 1.46 |
| | | yl]piperidine-2,6-dione | | (s, 1H), 1.31 (s, 3H) |
| 205 | I-142 | 3-[3-methyl-2-oxo-5-[3- | 874.45 | (400 MHz, CD₃OD) δ 8.76 (s, 1H), |
| | | (3-[4-[(4-[7-[trans-4- | | 7.63 (d, J = 7.7 Hz, 2H), 7.54-7.34 |
| | | hydroxy-4- | | (m, 3H), 7.10-6.93 (m, 3H), 5.40- |
| | | methylcyclohexyl]-2- | | 5.24 (m, 1H), 4.57 (s, 1H), 3.73 (t, |
| | | [(3,3,3- | | J = 7.3 Hz, 2H), 3.59 (s, 2H), 3.54- |
| | | trifluoropropyl)amino] | | 3.39 (m, 7H), 2.99-2.41 (m, 16H), |
| | | pyrrolo[2,3-d]pyrimidin-5- | | 2.22-2.15 (m, 3H), 2.08-1.70 (m, |
| | | yl]phenyl)methyl]piperazin- | | 10H), 1.44 (s, 3H) |
| | | 1-yl]propoxy)propyl]- | | |
| | | 1,3-benzodiazol-1- | | |
| | | yl]piperidine-2,6-dione | | |
| 206 | I-139 | 3-[3-methyl-2-oxo-5-[3- | 860.35 | (400 MHz, CD₃OD) δ 8.79 (s, 1H), |
| | | (3-[4-[(3-[7-[trans-4- | | 7.64 (s, 1H), 7.56 (d, J = 7.8 Hz, |
| | | hydroxycyclohexyl]-2- | | 1H), 7.46 (s, 1H), 7.40 (t, J = 7.7 |
| | | [(3,3,3- | | Hz, 1H), 7.23 (d, J = 7.5 Hz, 1H), |
| | | trifluoropropyl)amino] | | 7.07-6.94 (m, 3H), 5.32 (dd, J = |
| | | pyrrolo[2,3-d]pyrimidin-5- | | 12.2, 5.5 Hz, 1H), 4.57 (s, 1H), |
| | | yl]phenyl)methyl]piperazin- | | 3.77-3.69 (m, 3H), 3.62 (s, 2H), |
| | | 1-yl]propoxy)propyl]- | | 3.51-3.44 (m, 4H), 3.43 (s, 3H), |
| | | 1,3-benzodiazol-1- | | 2.96-2.73 (m, 6H), 2.70-2.53 (m, |
| | | yl]piperidine-2,6-dione | | 7H), 2.48 (t, J = 7.7 Hz, 2H), 2.19- |
| | | | | 2.03 (m, 8H), 1.95-1.87 (m, 2H), |
| | | | | 1.79 (t, J = 7.6 Hz, 2H), 1.56 (q, J = |
| | | | | 9.8, 9.0 Hz, 2H), 1.31 (s, 1H) |
| 207 | I-140 | 3-[3-methyl-2-oxo-5-(3- | 784.40 | (400 MHz, CD₃OD) δ 8.62 (s, 1H), |
| | | [3-[4-([7-[trans-4- | | 8.43 (s, 1H), 7.15 (s, 1H), 7.07- |
| | | hydroxycyclohexyl]-2- | | 7.00 (m, 2H), 6.97 (dd, J = 8.1, 1.5 |
| | | [(3,3,3- | | Hz, 1H), 5.33 (dd, J = 12.6, 5.4 Hz, |
| | | trifluoropropyl)amino] | | 1H), 4.47 (dq, J = 10.5, 5.7 Hz, |
| | | pyrrolo[2,3-d]pyrimidin-5- | | 1H), 3.86 (s, 2H), 3.75-3.63 (m, |
| | | yl]methyl)piperazin-1- | | 3H), 3.58-3.45 (m, 4H), 3.43 (d, J = |
| | | yl]propoxy]propyl)-1,3- | | 1.2 Hz, 3H), 3.08 (s, 3H), 3.01- |
| | | benzodiazol-1- | | 2.71 (m, 10H), 2.56 (qt, J = 11.2, |
| | | yl]piperidine-2,6-dione; | | 7.2 Hz, 2H), 2.23-1.84 (m, 10H), |
| | | formic acid | | 1.51 (qd, J = 12.2, 4.8 Hz, 2H) |
| 208 | I-170 | 3-(3-methyl-2-oxo-5-[2- | 858.45 | (400 MHz, CD₃OD) δ 8.76 (s, 1H), |
| | | [trans-3-[4-[(4-[7-[trans- | | 8.51 (s, 1H), 7.67 (d, J = 7.9 Hz, |
| | | 4-hydroxycyclohexyl]-2- | | 2H), 7.50-7.41 (m, 3H), 7.10 (s, |
| | | [(3,3,3- | | 1H), 7.06-6.98 (m, 2H), 5.33 (dd, J = |
| | | trifluoropropyl)amino] | | 12.3, 5.4 Hz, 1H), 4.57 (p, J = 8.6 |
| | | pyrrolo[2,3-d]pyrimidin-5- | | Hz, 1H), 4.07 (d, J = 6.8 Hz, 1H), |
| | | yl]phenyl)methyl]piperazin- | | 3.84 (s, 2H), 3.80-3.68 (m, 3H), |
| | | 1- | | 3.62 (t, J = 6.7 Hz, 2H), 3.44 (s, |
| | | yl]cyclobutoxy]ethyl]- | | 3H), 2.94 (d, J = 6.8 Hz, 3H), 2.92- |
| | | 1,3-benzodiazol-1- | | 2.83 (m, 6H), 2.83-2.76 (m, 4H), |
| | | yl)piperidine-2,6-dione; | | 2.58 (tt, J = 11.2, 7.2 Hz, 2H), 2.26 |
| | | formic acid | | (dt, J = 13.5, 6.8 Hz, 2H), 2.22- |
| | | | | 2.03 (m, 9H), 1.59-1.48 (m, 2H) |
| 209 | I-143 | 3-[3-methyl-2-oxo-5-[2- | 858.45 | (400 MHz, CD₃OD) δ 8.76 (s, 1H), |
| | | (3-[4-[(4-[7-[trans-4- | | 8.51 (s, 1H), 7.64 (d, J = 7.9 Hz, |
| | | hydroxycyclohexyl]-2- | | 2H), 7.48-7.38 (m, 3H), 7.08 (s, |
| | | [(3,3,3- | | 1H), 7.06-6.97 (m, 2H), 5.32 (dd, J = |
| | | trifluoropropyl)amino] | | 12.4, 5.3 Hz, 1H), 4.61-4.52 (m, |
| | | pyrrolo[2,3-d]pyrimidin-4- | | 1H), 3.83-3.68 (m, 6H), 3.63 (t, J = |
| | | yl]phenyl)methyl]piperazin- | | 6.6 Hz, 3H), 3.43 (s, 3H), 3.43- |
| | | 1- | | 3.40 (m, 2H), 2.99-2.86 (m, 3H), |
| | | yl]cyclobutoxy]ethyl]- | | 2.85-2.75 (m, 3H), 2.65 (s, 2H), |
| | | 1,3-benzodiazol-1- | | 2.64-2.56 (m, 5H), 2.55-2.43 (m, |
| | | yl]piperidine-2,6-dione; | | 3H), 2.18-2.06 (m, 7H), 1.80 (d, J = |
| | | formic acid | | 9.5 Hz, 2H), 1.59-1.50 (m, 2H) |
| 210 | I-144 | 3-(3-methyl-2-oxo-5-[2- | 858.40 | (400 MHz, CD₃OD) δ 8.77 (s, 1H), |
| | | [1-[1-[(4-[7-[trans-4- | | 8.50 (s, 1H), 7.68 (d, J = 7.9 Hz, |
| | | hydroxycyclohexyl]-2- | | 2H), 7.49 (s, 1H), 7.44 (d, J = 7.9 |
| | | [(3,3,3- | | Hz, 2H), 7.10 (s, 1H), 7.08-6.98 |
| | | trifluoropropyl)amino] | | (m, 2H), 5.33 (dd, J = 12.3, 5.4 Hz, |
| | | pyrrolo[2,3-d]pyrimidin-5- | | 1H), 4.61-4.53 (m, 1H), 4.25-4.18 |
| | | yl]phenyl)methyl] | | (m, 1H), 3.95-3.80 (s, 4H), 3.79- |
| | | piperidin-4-yl]azetidin-3- | | 3.62 (m, 6H), 3.44 (s, 3H), 3.23- |
| | | yl)oxy]ethyl]-1,3- | | 5.15 (m, 2H), 2.98-2.87 (m, 3H), |
| | | benzodiazol-1- | | 2.89-2.75 (m, 2H), 2.60-2.46 (m, |
| | | yl)piperidine-2,6-dione; | | 5H), 2.15-2.08 (m, 7H), 1.90 (d, J = |
| | | formic acid | | 13.1 Hz, 2H), 1.59-1.44 (m, 5H) |
| 211 | I-135 | 3-(3-methyl-2-oxo-4-[3- | 890.40 | (400 MHz, CD₃OD) δ 8.75 (s, 1H), |
| | | [2-(2-[4-[(4-[7-[trans-4- | | 7.58 (d, J = 7.9 Hz, 2H), 7.43 (s, |
| | | hydroxycyclohexyl]-2- | | 1H), 7.35 (d, J = 7.9 Hz, 2H), 7.06- |
| | | [(3,3,3- | | 6.99 (m, 1H), 6.95 (dd, J = 7.9, 4.1 |
| | | trifluoropropyl)amino] | | Hz, 2H), 5.32 (m, 1H), 4.62-4.52 |
| | | pyrrolo[2,3-d]pyrimidin-5- | | (m, 1H), 3.80-3.68 (m, 3H), 3.68- |
| | | yl]phenyl)methyl] | | 3.57 (m, 5H), 3.57-3.50 (m, 9H), |
| | | piperazin-1- | | 3.06 (m, 6.6 Hz, 2H), 3.00-2.77 |
| | | yl]ethoxy)ethoxy]propyl]- | | (m, 4H), 2.76-2.49 (m, 11H), 2.18- |
| | | 1,3-benzodiazol-1- | | 2.03 (m, 7H), 1.98-1.86 (m, 2H), |
| | | yl)piperidine-2,6-dione | | 1.54 (m, 2H) |
| 212 | I-155 | 3-[3-methyl-2-oxo-5-[2- | 886.45 | (400 MHz, CD₃OD) δ 8.80 (s, 1H), |
| | | ([1-[(4-[7-[trans-4- | | 8.21 (s, 1H), 7.67 (d, J = 7.5 Hz, |
| | | hydroxycyclohexyl]-2- | | 2H), 7.49 (s, 1H), 7.42 (d, J = 7.8 |
| | | [(3,3,3- | | Hz, 2H), 7.12 (s, 1H), 7.03 (s, 2H), |
| | | trifluoropropyl)amino] | | 5.27 (s, 1H), 4.57 (s, 1H), 3.82- |
| | | pyrrolo[2,3-d]pyrimidin-5- | | 3.57 (m, 9H), 3.44 (s, 3H), 2.94- |
| | | yl]phenyl)methyl]-[1,4- | | 2.82 (m, 7H), 2.83-2.75 (m, 4H), |
| | | bipiperidin]-4- | | 2.60 (dt, J = 11.2, 7.3 Hz, 2H), |
| | | yl]oxy)ethyl]-1,3- | | 2.28 (s, 2H), 2.19-2.05 (m, 7H), |
| | | benzodiazol-1- | | 1.95-1.82 (m, 6H), 1.69-1.58 (m, |
| | | yl]piperidine-2,6-dione; | | 4H) |
| | | formic acid | | |
| 213 | I-157 | 3-[3-methyl-2-oxo-5- | 872.50 | (400 MHz, CD₃OD) δ 8.76 (s, 1H), |
| | | [([1-[(4-[7-[trans-4- | | 8.53 (s, 1H), 7.64 (d, J = 8.0 Hz, |
| | | hydroxycyclohexyl]-2- | | 2H), 7.49-7.36 (m, 3H), 7.22 (d, J = |
| | | [(3,3,3- | | 1.4 Hz, 1H), 7.18-7.08 (m, 2H), |
| | | trifluoropropyl)amino] | | 5.35 (m, 1H), 4.64 (s, 2H), 4.57 (p, |
| | | pyrrolo[2,3-d]pyrimidin-5- | | J = 8.5 Hz, 1H), 3.72 (m, 6H), 3.45 |
| | | yl]phenyl)methyl]-[1,4- | | (s, 3H), 3.26 (s, 2H), 3.17 (d, J = |
| | | bipiperidin]-4- | | 11.7 Hz, 2H), 3.10-2.89 (m, 4H), |
| | | yl]oxy)methyl]-1,3- | | 2.86-2.74 (m, 2H), 2.65-2.54 (m, |
| | | benzodiazol-1- | | 2H), 2.32-2.12 (m, 5H), 2.12-1.89 |
| | | yl]piperidine-2,6-dione; | | (m, 8H), 1.96 (s, 2H), 1.77 (m, |
| | | formic acid | | 2H), 1.55 (m, 2H) |
| 214 | I-156 | 3-[3-methyl-2-oxo-5-[3- | 856.45 | (400 MHz, CD₃OD) δ 8.76 (s, 1H), |
| | | (1-[1-[(4-[7-[trans-4- | | 8.44 (s, 1H), 7.64 (d, J = 8.1 Hz, |
| | | hydroxycyclohexyl]-2- | | 2H), 7.45 (s, 1H), 7.41 (d, J = 7.9 |
| | | [(3,3,3- | | Hz, 2H), 7.07-7.00 (m, 2H), 6.97 |
| | | trifluoropropyl)amino] | | (dd, J = 8.1, 1.6 Hz, 1H), 5.33 (dd, |
| | | pyrrolo[2,3-d]pyrimidin-5- | | J = 12.5, 5.4 Hz, 1H), 4.63-4.50 |
| | | yl]phenyl)methyl] | | (m, 1H), 4.09 (t, J = 9.0 Hz, 2H), |
| | | piperidin-4-yl]azetidin-3- | | 3.79-3.69 (m, 5H), 3.65 (t, J = 8.9 |
| | | yl)propyl]-1,3- | | Hz, 2H), 3.43 (s, 3H), 3.11 (d, J = |
| | | benzodiazol-1- | | 11.9 Hz, 2H), 3.02 (s, 1H), 2.95 (s, |
| | | yl]piperidine-2,6-dione; | | 1H), 2.87-2.69 (m, 5H), 2.68-2.60 |
| | | formic acid | | (m, 1H), 2.57 (dt, J = 11.2, 5.7 Hz, |
| | | | | 1H), 2.30-2.22 (m, 2H), 2.17 (dq, J = |
| | | | | 6.4, 3.4 Hz, 2H), 2.15-2.08 (m, |
| | | | | 3H), 2.07 (d, J = 6.4 Hz, 2H), 1.98 |
| | | | | (d, J = 12.4 Hz, 2H), 1.72-1.63 (m, |
| | | | | 4H), 1.59-1.41 (m, 4H) |
| 215 | I-145 | 3-[3-methyl-2-oxo-5-(3- | 884.50 | (400 MHz, CD₃OD) δ 8.75 (s, 1H), |
| | | [1-[(4-[7-[trans-4- | | 8.55 (s, 1H), 7.63 (d, J = 8.1 Hz, |
| | | hydroxycyclohexyl]-2- | | 2H), 7.44 (s, 1H), 7.40 (d, J = 7.9 |
| | | [(3,3,3- | | Hz, 2H), 7.06-6.99 (m, 2H), 6.96 |
| | | trifluoropropyl)amino] | | (dd, J = 8.2, 1.5 Hz, 1H), 5.32 (m, |
| | | pyrrolo[2,3-d]pyrimidin-5- | | 1H), 4.61-4.50 (m, 1H), 3.79-3.68 |
| | | yl]phenyl)methyl]-[1,4- | | (m, 3H), 3.62 (s, 2H), 3.49-3.34 |
| | | bipiperidin]-4-yl]propyl)- | | (m, 5H), 3.12 (d, J = 11.7 Hz, 2H), |
| | | 1,3-benzodiazol-1- | | 3.01-2.88 (m, 2H), 2.88-2.76 (m, |
| | | yl]piperidine-2,6-dione; | | 4H), 2.71 (t, J = 7.4 Hz, 2H), 2.58 |
| | | formic acid | | (m, 2H), 2.23-2.01 (m, 11H), 1.96 |
| | | | | (m, 2H), 1.79-1.65 (m, 4H), 1.59- |
| | | | | 1.50 (m, 3H), 1.36 (t, J = 8.0 Hz, |
| | | | | 4H) |
| 216 | I-163 | 3-[3-methyl-2-oxo-5-(3- | 867.50 | (400 MHz, CDCl₃) δ 10.19 (s, 1H), |
| | | [2-[(1-[[trans-4-[7- | | 8.59 (s, 1H), 6.95-6.86 (m, 2H), |
| | | [trans-4- | | 6.75 (d, J = 8.0 Hz, 1H), 6.64 (s, |
| | | hydroxycyclohexyl]-2- | | 1H), 5.69 (s, 1H), 5.22 (dd, J = |
| | | [(3,3,3- | | 12.7, 5.3 Hz, 1H), 4.43 (ddt, J = |
| | | trifluoropropyl)amino] | | 11.8, 7.8, 4.0 Hz, 1H), 3.79-3.65 |
| | | pyrrolo[2,3-d]pyrimidin-5- | | (m, 3H), 3.64-3.60 (m, 4H), 3.52 |
| | | yl]cyclohexyl]methyl] | | (t, J = 6.3 Hz, 2H), 3.45 (s, 3H), |
| | | piperidin-4- | | 3.31 (d, J = 27.2 Hz, 1H), 3.00- |
| | | yl)oxy]ethoxy]propyl)- | | 2.91 (m, 1H), 2.91-2.65 (m, 7H), |
| | | 1,3-benzodiazol-1- | | 2.53 (qt, J = 11.0, 7.1 Hz, 2H), |
| | | yl]piperidine-2,6-dione | | 2.43-1.99 (m, 9H), 1.98-1.70 (m, |
| | | | | 12H), 1.68-1.51 (m, 8H) |
| 217 | I-171 | 3-[3-methyl-2-oxo-5-(3- | 867.50 | (400 MHz, CDCl₃) δ 8.57 (s, 1H), |
| | | [2-[(1-[[cis-4-[7-[trans-4- | | 7.03-6.86 (m, 2H), 6.75 (d, J = 8.0 |
| | | hydroxycyclohexyl]-2- | | Hz, 1H), 6.61 (s, 1H), 5.58 (s, 1H), |
| | | [(3,3,3- | | 5.24 (d, J = 11.8 Hz, 1H), 4.44 (s, |
| | | trifluoropropyl)amino] | | 1H), 3.74 (d, J = 7.4 Hz, 3H), 3.63 |
| | | pyrrolo[2,3-d]pyrimidin-5- | | (d, J = 13.1 Hz, 4H), 3.52 (t, J = |
| | | yl]cyclohexyl]methyl] | | 6.3 Hz, 2H), 3.49-3.31 (m, 4H), |
| | | piperidin-4- | | 3.13-2.37 (m, 11H), 2.24-1.80 (m, |
| | | yl)oxy]ethoxy]propyl)- | | 18H), 1.63-1.45 (m, 9H), 1.10 (s, |
| | | 1,3-benzodiazol-1- | | 2H) |
| | | yl]piperidine-2,6-dione | | |
| 218 | I-172 | 3-[3-methyl-2-oxo-5-(3- | 881.75 | (400 MHz, CDCl₃) δ 8.58 (s, 1H), |
| | | [2-[(1-[[trans-4-[7- | | 6.92 (d, J = 8.9 Hz, 2H), 6.74 (d, J = |
| | | [trans-4- | | 7.9 Hz, 1H), 6.63 (s, 1H), 5.63 |
| | | hydroxycyclohexyl]-2- | | (s, 1H), 5.24 (dd, J = 12.6, 5.4 Hz, |
| | | [(3,3,3- | | 1H), 4.44 (s, 1H), 3.74 (q, J = 6.7 |
| | | trifluoropropyl)amino] | | Hz, 3H), 3.69-3.58 (m, 4H), 3.52 |
| | | pyrrolo[2,3-d]pyrimidin-5- | | (t, J = 6.2 Hz, 2H), 3.46 (s, 3H), |
| | | yl]cyclohexyl]methyl] | | 3.34 (d, J = 6.3 Hz, 2H), 3.18-2.64 |
| | | piperidin-4- | | (m, 8H), 2.62-2.46 (m, 2H), 2.42- |
| | | yl)methoxy]ethoxy] | | 2.02 (m, 7H), 1.95-1.85 (m, 7H), |
| | | propyl)-1,3-benzodiazol-1- | | 1.87-1.70 (m, 4H), 1.73-1.47 (m, |
| | | yl]piperidine-2,6-dione | | 11H), 1.28 (s, 2H) |
| 219 | I-164 | 3-[3-methyl-2-oxo-5-(3- | 895.55 | (400 MHz, CDCl₃) δ 8.58 (s, 1H), |
| | | [2-[2-(1-[[trans-4-[7- | | 6.93 (d, J = 7.0 Hz, 2H), 6.73 (d, J = |
| | | [trans-4- | | 8.0 Hz, 1H), 6.64 (s, 1H), 5.67 |
| | | hydroxycyclohexyl]-2- | | (s, 1H), 5.25 (dd, J = 13.0, 5.2 Hz, |
| | | [(3,3,3- | | 1H), 4.42 (d, J = 12.7 Hz, 1H), |
| | | trifluoropropyl)amino] | | 3.79-3.69 (m, 3H), 3.68-3.60 (m, |
| | | pyrrolo[2,3-d]pyrimidin-5- | | 4H), 3.56-3.50 (m, 3H), 3.45 (s, |
| | | yl]cyclohexyl]methyl] | | 3H), 2.97-2.70 (m, 7H), 2.63-2.01 |
| | | piperidin-4- | | (m, 10H), 1.99-1.81 (m, 7H), 1.76 |
| | | yl)ethoxy]ethoxy]propyl)- | | (s, 5H), 1.71-1.49 (m, 11H), 1.47- |
| | | 1,3-benzodiazol-1- | | 1.35 (m, 4H) |
| | | yl]piperidine-2,6-dione | | |
| 220 | I-158 | 3-[3-methyl-2-oxo-5-([4- | 843.50 | (400 MHz, CD₃OD) δ 8.75 (s, 1H), |
| | | [(3R)-1-[(4-[7-[trans-4- | | 7.71-7.53 (m, 3H), 7.46-7.37 (m, |
| | | hydroxycyclohexyl]-2- | | 3H), 7.19 (s, 1H), 7.13-7.04 (m, |
| | | [(3,3,3- | | 2H), 5.39-5.29 (m, 1H), 4.56 (p, J = |
| | | trifluoropropyl)amino] | | 8.2 Hz, 1H), 3.73 (t, J = 7.3 Hz, |
| | | pyrrolo[2,3-d]pyrimidin-5- | | 4H), 3.69-3.62 (m, 1H), 3.61 (s, |
| | | yl]phenyl)methyl]pyrrolidin- | | 2H), 3.44 (s, 3H), 2.99-2.89 (m, |
| | | 3-yl]piperazin-1- | | 3H), 2.88-2.75 (m, 3H), 2.69-2.40 |
| | | yl]methyl)-1,3- | | (m, 11H), 2.22-2.04 (m, 8H), 1.80 |
| | | benzodiazol-1- | | (s, 1H), 1.56-1.50 (m, 2H), 1.31 (s, |
| | | yl]piperidine-2,6-dione | | 1H) |
| 221 | I-159 | 3-(3-methyl-2-oxo-4-[[4- | 870.50 | (400 MHz, DMSO-d₆) δ 11.09 (s, |
| | | ([1-[(4-[7-[trans-4- | | 1H), 8.88 (s, 1H), 7.63 (d, J = 6.6 |
| | | hydroxycyclohexyl]-2- | | Hz, 3H), 7.30 (d, J = 7.7 Hz, 2H), |
| | | [(3,3,3- | | 7.05 (q, J = 6.8, 5.9 Hz, 2H), 6.95 |
| | | trifluoropropyl)amino] | | (t, J = 7.7 Hz, 1H), 6.86 (d, J = 7.6 |
| | | pyrrolo[2,3-d]pyrimidin-5- | | Hz, 1H), 5.37 (m, 1H), 4.69 (d, J = |
| | | yl]phenyl)methyl]piperidin- | | 4.4 Hz, 1H), 4.43 (t, J = 7.9 Hz, |
| | | 4-yl]methyl)piperidin- | | 1H), 3.67 (s, 3H), 3.67-3.51 (m, |
| | | 1-yl]methyl]-1,3- | | 5H), 3.43 (s, 2H), 2.84-2.60 (m, |
| | | benzodiazol-1- | | 8H), 2.09-1.89 (m, 11H), 1.61- |
| | | yl)piperidine-2,6-dione | | 1.54 (m, 4H), 1.36 (m, 5H), 1.15- |
| | | | | 1.00 (m, 6H) |

### Step 2: 3-[6-([2-[2-(2-[4-[(4-[7-[trans-4-hydroxycyclohexy]-2-[(3,3,3-trifluoropropyl)amino]pyrrolo[2,3-d]pyrimidin-5-yl]phenyl)methyl]piperazin-1-yl]ethoxy)ethoxy]ethyl]amino)pyrido[2,3-b]indol-9-yl]piperidine-2,6-dione; formic acid

To a stirred solution of tert-butyl N-[9-(2,6-dioxopiperidin-3-yl)pyrido[2,3-b]indol-6-yl]-N-[2-[2-(2-[4-[(4-[7-[trans-4-hydroxycyclohexyl]-2-[(3,3,3-trifluoropropyl)amino]pyrrolo[2,3-d]pyrimidin-5-yl]phenyl)methyl]piperazin-1-yl]ethoxy)ethoxy]ethyl]carbamate (20.0 mg, 0.02 mmol) in DCM (10.0 mL) was added HCl (4M) in 1,4-dioxane (2.00 mL) at room temperature under nitrogen atmosphere. The reaction was reacted for 2 h and was concentrated under reduced pressure. The crude product (50 mg) was purified by Prep-HPLC with the following conditions (Column: Xselect CSH OBD Column 30×150 mm 5 um; Mobile Phase A: water (plus 0.1% FA), Mobile Phase B: ACN; Flow rate: 60 mL/min; Gradient: 13% B to 22% B in 7 min; detector: UV 220/254 nm; desired fractions were collected at 6.48 min and lyophilized to afford trans-3-[6-[14-(4-[7-[trans-4-hydroxycyclohexyl]-2-[(3,3,3-trifluoropropyl)amino]pyrrolo[2,3-d]pyrimidin-5-yl]phenyl)-4,7-dioxa-1,10,13-triazatetradecan-1-yl]pyrido[2,3-b]indol-9-yl]piperidine-2,6-dione; formic acid (10.0 mg, 54%) as a white solid. ¹H NMR (400 MHz, CD₃OD) δ 8.73 (s, 1H), 8.50 (s, 1H), 8.38 (dd, J=7.7, 1.6 Hz, 1H), 8.34 (dd, J=4.9, 1.6 Hz, 1H), 7.57-7.51 (m, 2H), 7.49 (d, J=2.3 Hz, 1H), 7.41 (s, 1H), 7.32 (m, 1H), 7.28-7.21 (m, 2H), 7.17 (dd, J=7.6, 4.9 Hz, 1H), 7.01 (m, 2.3 Hz, 1H), 5.92-5.85 (m, 1H), 4.55 (p, J=8.1 Hz, 1H), 3.81-3.65 (m, 11H), 3.53 (s, 2H), 3.41 (t, J=5.2 Hz, 2H), 3.06-2.77 (m, 9H), 2.70-2.50 (m, 6H), 2.18-1.99 (m, 7H), 1.54 (m, 2H). LC/MS (ESI, m/z): [(M/2+1)]⁺=456.55.

The following examples in Table 54 were synthesized according to the above procedure:

TABLE 54

| | | | MS: | |
|---|---|---|---|---|
| Examples | I-# | Name | [(M + 1)]⁺ | ¹H NMR |
| 222 | I-68 | (2S,4R)-1-[(2S)-3,3-dimethyl- | 1044.55 | (400 MHz, CD₃OD) δ 9.76 (s, 1H), |
| | | 2-([2-[(5-[4-[(4-[7-[trans-4- | | 8.94 (s, 1H), 8.03 (s, 1H), 7.84 (d, |
| | | hydroxycyclohexyl]-2-[(3,3,3- | | J = 7.9 Hz, 2H), 7.77 (d, J = 7.8 Hz, |
| | | trifluoropropyl)amino]-7H- | | 2H), 7.60-7.50 (m, 4H), 4.78 (s, |
| | | pyrrolo[2,3-d]pyrimidin-5 | | 2H), 4.70-4.50 (m, 5H), 4.45-4.35 |
| | | yl]phenyl)methyl]piperazin-1- | | (m, 2H), 4.05 (d, J = 11.5 Hz, 1H), |
| | | yl]pentyl)amino]ethyl]ami- | | 3.88 (t, J = 6.9 Hz, 2H), 3.82-3.68 |
| | | no)butanoyl]-4-hydroxy-N-[[4- | | (m, 9H), 3.65-3.55 (m, 2H), 3.51 (s, |
| | | (4-methyl-1,3-thiazol-5- | | 2H), 3.37 (t, J = 6.5 Hz, 2H), 3.15 |
| | | yl)phenyl]methyl]pyrrolidine- | | (t, J = 7.5 Hz, 2H), 2.68 (dt, J = |
| | | 2-carboxamide dihydrochloride | | 11.0, 6.9 Hz, 2H), 2.59 (s, 3H), 2.37 |
| | | | | (dd, J = 13.4, 7.9 Hz, 1H), 2.21- |
| | | | | 2.03 (m, 7H), 1.93-1.81 (m, 4H), |
| | | | | 1.59 (s, 4H), 1.24 (s, 9H) |

### EXAMPLE 223: Trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-amino-3-methylbutanoate; formic acid (I-77)

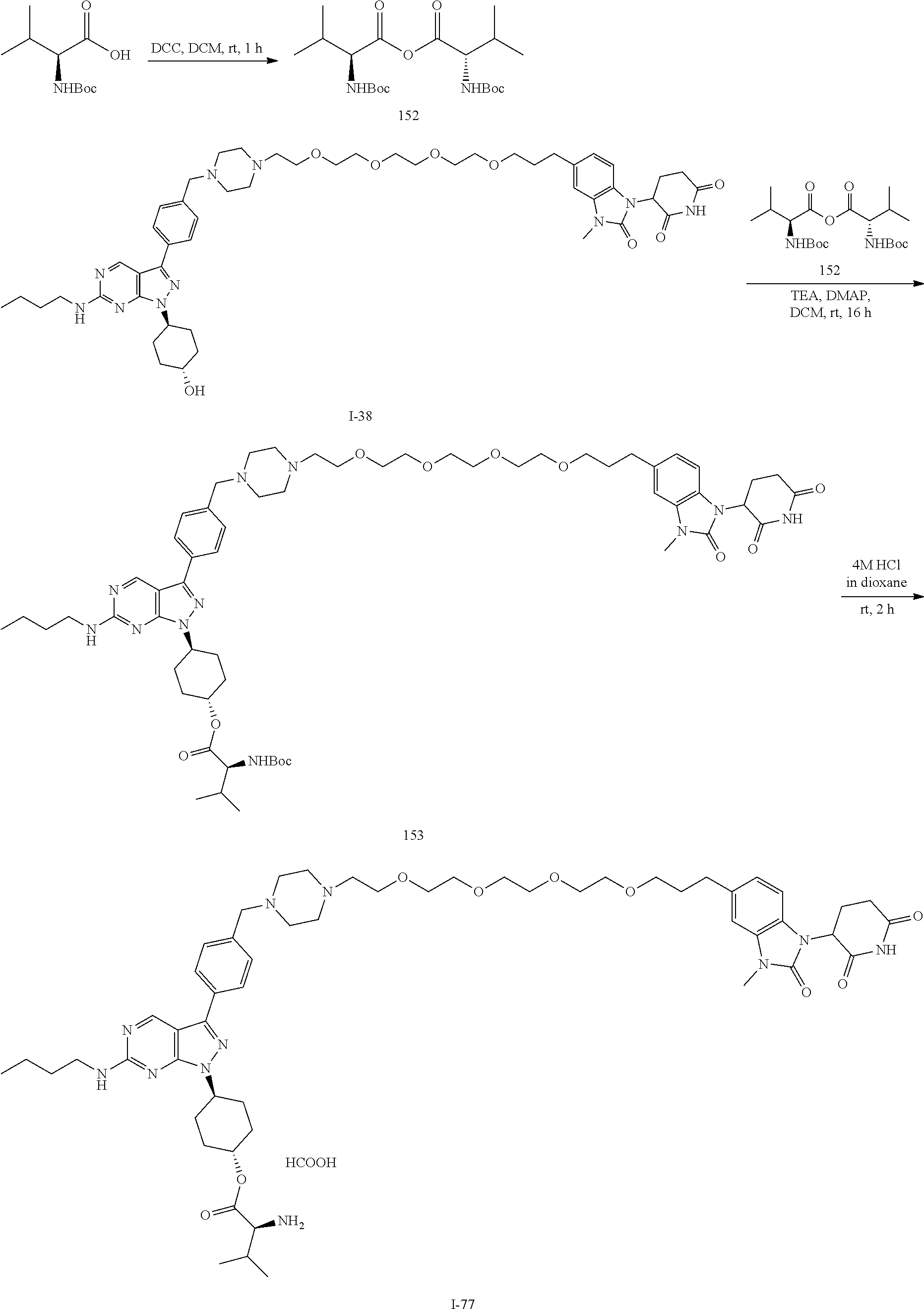

### Step 1: (S)-2-(tert-butoxycarbonylamino)-3-methylbutanoic anhydride

To a stirred solution of (2S)-2-[(tert-butoxycarbonyl)amino]-3-methylbutanoic acid (1.00 g, 4.61 mmol) in DCM (10.0 mL) was added DCC (0.52 g, 2.53 mmol) at room temperature under nitrogen atmosphere. The reaction was stirred for 1 h at room temperature. The reaction was quenched with water/ice (10 mL). The resulting mixture was extracted with CH₂C₂ (2×15.0 mL). The combined organic layers were washed with brine (20.0 mL) and dried over anhydrous Na₂SO₄. After filtration, the filtrate was concentrated under reduced pressure to give (S)-2-(tert-butoxycarbonylamino)-3-methylbutanoic anhydride (1.50 g, crude) as a white solid. ¹H NMR (400 MHz, DMSO-d₆) δ 6.91 (d, J=8.6 Hz, 2H), 3.78 (dd, J=8.7, 5.7 Hz, 2H), 2.06-2.00 (m, 2H), 1.36 (s, 18H), 0.88 (d, J=4.3 Hz, 12H).

### Step 2: trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-[[(tert-butoxy)carbony]amino]-3-methylbutanoate

To a stirred solution of 3-(5-[1-[4-([4-[6-(butylamino)-1-[trans-4-hydroxycyclohexyl]-1H-pyrazolo[3,4-d]pyrimidin-3-yl]phenyl]methyl)piperazin-1-yl]-3,6,9,12-tetraoxapentadecan-15-yl]-3-methyl-2-oxo-2,3-dihydro-1H-1,3-benzodiazol-1-yl)piperidine-2,6-dione (80.0 mg, 0.085 mmol), TEA (103 mg, 1.02 mmol) and DMAP (10.4 mg, 0.085 mmol) in DCM (5.00 mL) was added (S)-2-(tert-butoxycarbonylamino)-3-methylbutanoic anhydride (355 mg, 0.85 mmol) at 0° C. under nitrogen atmosphere. The solution was stirred for 16 h at room temperature. Upon completion, the solution was concentrated under reduced pressure and the residue was purified by Prep-HPLC with the following conditions (Column: XBridge Shield RP18 OBD Column 30×150 mm, 5 um; Mobile Phase A: water (0.1% NH₄HCO₃), Mobile Phase B: ACN; Flow rate: 60 mL/min; Gradient: 5% B to 33% B in 6 min; detector: UV 254/220 nm; desired fractions were collected at 6.08 min and lyophilized to afford trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-[[(tert-butoxy)carbonyl]amino]-3-methylbutanoate (50.0 mg, 52%) as a white solid. ¹H NMR (400 MHz, CDCl₃) δ 8.96 (s, 1H), 7.82 (d, J=7.7 Hz, 2H), 7.40 (s, 2H), 6.92 (dd, J=4.5, 2.9 Hz, 2H), 6.77 (d, J=8.3 Hz, 1H), 5.25 (dd, J=12.7, 5.3 Hz, 1H), 5.08 (d, J=9.0 Hz, 1H), 5.01-4.94 (m, 1H), 4.71-4.61 (m, 1H), 4.25 (dd, J=9.1, 4.6 Hz, 1H), 4.04 (s, 1H), 3.71-3.57 (m, 15H), 3.56-3.44 (m, 8H), 3.02-2.81 (m, 6H), 2.80-2.70 (m, 4H), 2.27 (m, 6H), 2.12 (m, 2H), 1.96-1.89 (m, 2H), 1.75-1.62 (m, 7H), 1.50 (s, 11H), 1.30 (m, 1H), 1.05-0.99 (m, 6H), 0.94 (d, J=6.9 Hz, 3H). LC/MS (ESI, m/z): [(M/2+1)]⁺=570.15.

The following example in Table 55 were synthesized according to the above procedure:

TABLE 55

| | | | MS: | |
|---|---|---|---|---|
| Example | I-# | Name | [(M + 1)]⁺ | ¹H NMR |
| 224 | I-65 | 3-(5-[1-[4-([4-[6- | 981.55 | (400 MHz, CD₃OD) δ 8.96 (s, 1H), 7.95- |
| | | (butylamino)-1-[trans- | | 7.88 (m, 2H), 7.48 (d, J = 7.8 Hz, 2H), 7.06- |
| | | 4-hydroxycyclohexyl]-1H- | | 6.98 (m, 2H), 6.95 (dd, J = 8.0, 1.5 Hz, 1H), |
| | | pyrazolo[3,4-d]pyrimidin-3- | | 5.31 (dd, J = 12.6, 5.4 Hz, 1H), 4.86-4.77 |
| | | yl]phenyl]methyl)piperazin- | | (m, 1H), 4.67 (d, J = 11.6 Hz, 1H), 3.69 (t, |
| | | 1-yl]-3,6,9,12-tetraoxapenta- | | J = 5.3 Hz, 2H), 3.67-3.58 (m, 13H), 3.55 |
| | | decan-15-yl]-3-methyl-2- | | (dd, J = 5.8, 3.2 Hz, 2H), 3.49 (t, J = 7.1 Hz, |
| | | oxo-2,3-dihydro-1H-1,3- | | 2H), 3.48-3.40 (m, 5H), 2.97-2.70 (m, 11H), |
| | | benzodiazol-1-yl)piperidine- | | 2.69-2.55 (m, 3H), 2.36-2.14 (m, 5H), 2.10- |
| | | 2,6-dione | | 2.05 (m, 5H), 1.93-1.82 (m, 2H), 1.74-1.60 |
| | | | | (m, 4H), 1.48 (h, J = 7.4 Hz, 2H), 1.02 (t, |
| | | | | J = 7.4 Hz, 3H) |

### Step 3: trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-amino-3-methylbutanoate; formic acid

To a stirred solution of trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-[[(tert-butoxy)carbonyl]amino]-3-methylbutanoate (50.0 mg, 0.044 mmol) in DCM (5.00 mL) was added HCl (4 M) in 1,4-dioxane (5.00 mL) dropwise at room temperature under nitrogen atmosphere. The solution was stirred for 2 h at room temperature. Upon completion, the solution was concentrated under reduced pressure and the residue was purified by Prep-HPLC with the following conditions (Column: Xselect CSH OBD Column 30×150 mm 5 um; Mobile Phase A: water (plus 0.1% FA), Mobile Phase B: ACN; Flow rate: 60 mL/min; Gradient: 10% B to 38% B in 8 min; detector: UV 220/254 nm; desired fractions were collected at 8.47 min and lyophilized to afford trans-4-[6-(butylamino)-3-[4-[(4-[15-[1-(2,6-dioxopiperidin-3-yl)-3-methyl-2-oxo-2,3-dihydro-1H-1,3-benzodiazol-5-yl]-3,6,9,12-tetraoxapentadecan-1-yl]piperazin-1-yl)methyl]phenyl]-1H-pyrazolo[3,4-d]pyrimidin-1-yl]cyclohexyl (2S)-2-amino-3-methylbutanoate; formic acid (40.0 mg, 84%) as a white solid. ¹H NMR (400 MHz, CD₃OD) δ 8.97 (s, 1H), 8.52 (s, 1H), 7.92 (d, J=8.1 Hz, 2H), 7.49 (d, J=8.2 Hz, 2H), 7.06-6.92 (m, 3H), 5.32 (m, 1H), 5.00 (s, 1H), 4.72 (s, 1H), 3.71 (t, J=4.4 Hz, 3H), 3.68-3.60 (m, 13H), 3.55 (dd, J=5.5, 3.3 Hz, 2H), 3.49 (t, J=7.1 Hz, 2H), 3.46-3.40 (m, 5H), 3.01-2.86 (m, 6H), 2.85-2.60 (m, 8H), 2.38-2.19 (m, 5H), 2.17-2.06 (m, 3H), 1.87 (p, J=6.8 Hz, 2H), 1.76 (q, J=9.6, 7.4 Hz, 2H), 1.71-1.63 (m, 2H), 1.48 (h, J=7.3 Hz, 2H), 1.08 (dd, J=6.9, 3.2 Hz, 6H), 1.02 (t, J=7.4 Hz, 3H). LC/MS (ESI, m/z): [(M/2 +1)]⁺=1038.60.

### EXAMPLE 225: MerTK Degradation In Vitro

### Step 1: Compound Treatment:

Compounds are reconstituted in DMSO to make 10 mM stock solutions. Cells are maintained in Roswell Park Memorial Institute (RPMI)-1640 medium containing 10% fetal bovine serum (FBS). Cells are seeded into 6-well plates with about 2-5×10⁶ cells per well. 2 μL of diluted compounds are added to the cells to reach final concentrations of 0.001-10 μM. After a 4 or a 24 hour-incubation at 37° C., cells are either:
1) collected into 2 mL Eppendorf tubes and centrifuged at 1,000 rpm for 5 min, washed with Dulbecco's Phosphate-Buffered Saline (DPBS; 1×) once, and resuspended in 60 μL RIPA lysis buffer (Thermo Fisher, 89900) with Halt Protease and Phosphatase Inhibitor Cocktail (Thermo Fisher, 78446); or
2) Ripa lysis buffer with Halt Protease and Phosphatase Inhibitor Cocktail is added directly to the cells; and
   the cells are lysed on ice for 10 min, then centrifuged at 14,000 rpm for 10 min at 4° C. and the supernatants are collected for western blots.

### Step 2: Protein Concentration Determination:

The protein concentration of cell lysates is quantified with Pierce™ BCA Protein Assay Kit (Pierce, 23227). Albumin standards at different concentrations are prepared, involving 2,000 μg/mL, 1,500 μg/mL, 1,000 μg/mL, 750 μg/mL, 500 μg/mL, 250 μg/mL, 125 μg/mL, and 25 μg/mL. BCA working reagents are prepared by mixing BCA reagent A with reagent B in 50:1 ratio. 200 μL of the BCA working reagents is added to 25 μL of BCA standard or cell lysates in microplate, and mixed thoroughly on a plate shaker for 30 seconds. After incubation at 37° C. for 30 min, the absorbance of samples at 562 nm is measured with EnVision Plate Reader.

### Step 3: Western Blot Analysis:

Protein lysates are prepared in NuPAGE™ LDS sample buffer and NuPAGE™ sample reducing agent, and incubated at 95° C. for 5 min. For Western blots, 20-25 μg of total proteins is resolved in 4-12% Bis-Tris gels (Invitrogen, WG1403A) with 1× IVIES SDS running buffer (Invitrogen, NP0002). The proteins are transferred to low fluorescence PVDF membranes using the Trans-Blot Turbo Transfer System. Membranes are then blocked in Odyssey blocking buffer at RT for 1 h followed by primary incubation at 4° C. overnight. The primary antibodies are MerTK rabbit monoclonal antibody (CST, #4319, 1:1000), and actin mouse monoclonal antibody (Licor, 926-24412, 1:10,000). Membranes are washed three times with Tris Buffered Saline with Tween-20 (TBST; 1×), and then incubated with IR Dye 800 CW Goat anti-rabbit (Licor, #926-32211) and IR Dye 700 CW Goat anti-mouse (Licor, #926-68070) secondary antibodies in 1:10,000 dilution at RT for 1 h. The Western blot images are obtained using Odyssey Imaging System.

### EXAMPLE 226: MerTk Degradation in Jurkat Cells

### Step 1: Compound Treatment:

Compounds were reconstituted in DMSO to make 10 mM stock solutions. Jurkat cells were maintained in RPMI-1640 medium containing 10% FBS. Cells were seeded into 6-well plates with about 5×10⁶ cells per well. 2 μL of diluted compounds were added to cells to the final concentration of 0.001-1 μM. After a 4 hour-incubation at 37° C., cells were collected into 2 mL Eppendorf tubes and centrifuged at 1,000 rpm for 5 min. The cell pellets were washed with DPBS (1×) once and resuspended in 60 μL RIPA lysis buffer with Halt Protease and Phophatase Inhibitor Cocktail. The cells were lysed on ice for 10 min, then centrifuged at 14,000 rpm for 10 min at 4° C. and the supernatants were collected for western blots.

### Step 2: Protein Concentration Determination:

The protein concentration of cell lysates was quantified with Pierce™ BCA Protein Assay Kit. Albumin standards at different concentrations were prepared, involving 2,000 μg/mL, 1,500 μg/mL, 1,000 μg/mL, 750 μg/mL, 500 μg/mL, 250 μg/mL, 125 μg/mL, and 25 μg/mL. BCA working reagents were prepared by mixing BCA reagent A with reagent B in 50:1 ratio. 200 μL of the BCA working reagents were added to 25 ∥L of BCA standard or cell lysates in microplate, and mixed thoroughly on a plate shaker for 30 seconds. After incubation at 37° C. for 30 min, the absorbance of samples at 562 nm were measured with EnVision Plate Reader.

### Step 3: Western Blot Analysis:

Protein lysates were prepared in NuPAGE™ LDS sample buffer and NuPAGE™ sample reducing agent, and incubated at 95° C. for 5 min. For Western blots, 20-25 μg of total proteins were resolved in 4-12% Bis-Tris gels (Invitrogen, WG1403A) with 1× MES SDS running buffer. The proteins were transferred to low fluorescence PVDF membranes using the Trans-Blot Turbo Transfer System. Membranes were then blocked in Odyssey blocking buffer at RT for 1 h followed by primary incubation at 4° C. overnight. The primary antibodies were MerTK rabbit monoclonal antibody and actin mouse monoclonal antibody. Membranes were washed three times with TBST (1×), and then incubated with IR Dye 800 CW Goat anti-rabbit and IR Dye 700 CW Goat anti-mouse secondary antibodies in 1:10,000 dilution at RT for 1 h. The Western blot images were obtained using Odyssey Imaging System.

### Results:

Degradation results are summarized in Table 2, below. Compounds that elicited a maximum MerTK degradation greater than 50% are designated “A.” Compounds that elicited a maximum MerTK degradation greater than 20% (>20%) to less than or equal to 50% (≤50%)) are designated “B.” Compounds that elicited a maximum MerTK degradation less than or equal to 20% (≤20%) are designated “C.” “N/A” indicates not assayed.

### EXAMPLE 227: MerTK Degradation in RAW264 Cells

### Step 1: Compound Treatment:

Compounds were reconstituted in DMSO to make 10 mM stock solutions. RAW264 cells were maintained in DMEM medium containing 10% FBS. Cells were seeded into 6-well plates with 2×10⁶ cells per well. 2 μL of diluted compounds were added to cells to the final concentration of 0.001-1 μM. After a 4 hour-incubation at 37° C., cells were washed with DPBS (1×) once. Then RIPA lysis buffer with Halt Protease and Phosphatase Inhibitor Cocktail was added directly to the cells and cells were then incubated on ice for 10 min, then lysate was harvested into 1.5 mL Eppendorf tubes and centrifuged at 14,000 rpm for 10 min at 4° C. and the supernatants were collected for Western blots.

### Step 2:Protein Concentration Determination:

The protein concentration of cell lysates was quantified with Pierce™ BCA Protein Assay Kit. Albumin standards at different concentrations were prepared, involving 2,000 μg/mL, 1,500 μg/mL, 1,000 μg/mL, 750 μg/mL, 500 μg/mL, 250 μg/mL, 125 μg/mL, and 25 μg/mL. BCA working reagents were prepared by mixing BCA reagent A with reagent B in 50:1 ratio. 200 μL of the BCA working reagents were added to 25 μL of BCA standard or cell lysates in microplate, and mixed thoroughly on a plate shaker for 30 seconds. After incubation at 37° C. for 30 min, the absorbance of samples at 562 nm were measured with EnVision Plate Reader.

### Step 3: Western Blot Analysis

Protein lysates were prepared in NuPAGE™ LDS sample buffer and NuPAGE™ sample reducing agent, and incubated at 95° C. for 5 min. For Western blots, 20-25 μg of total proteins were resolved in 4-12% Bis-Tris gels with 1 × MES SDS running buffer. The proteins were transferred to low fluorescence PVDF membranes using the Trans-Blot Turbo Transfer System. Membranes were then blocked in Odyssey blocking buffer at RT for 1 h followed by primary incubation at 4° C. overnight. The primary antibodies were MerTK goat polyclonal antibody and actin mouse monoclonal antibody. Membranes were washed three times with TBST (1×), and then incubated with IR Dye 800 CW Donkey anti-goat and IR Dye 700 CW Goat anti-mouse secondary antibodies in 1:10,000 dilution at RT for 1 h. The Western blot images were obtained using Odyssey Imaging System.

### Results:

Degradation results are summarized in Table 2, below. Compounds that elicited a maximum MerTK degradation greater than 50% are designated “A.” Compounds that elicited a maximum MerTK degradation greater than 20% (>20%) to less than or equal to 50% (≤50%)) are designated “B.” Compounds that elicited a maximum MerTK degradation less than or equal to 20% (≤20%) are designated “C.” “N/A” indicates not assayed.

TABLE 2
In Vitro MerTK Degradation

| Compound | Jurkat Max % | RAW264 Max % |
|---|---|---|
| Number | MerTK Degradation | MerTK Degradation |
| I-1 | A | N/A |
| I-2 | A | N/A |
| I-3 | C | N/A |
| I-4 | A | N/A |
| I-5 | C | N/A |
| I-6 | A | A |
| I-7 | A | A |
| I-8 | A | N/A |
| I-9 | A | A |
| I-10 | A | A |
| I-11 | A | N/A |
| I-12 | A | A |
| I-13 | A | A |
| I-14 | A | A |
| I-15 | A | B |
| I-19 | A | A |
| I-20 | A | A |
| I-21 | B | B |
| I-22 | A | N/A |
| I-23 | A | B |
| I-24 | A | A |
| I-25 | A | A |
| I-26 | A | A |
| I-27 | A | A |
| I-28 | A | A |
| I-29 | A | A |
| I-31 | B | N/A |
| I-32 | A | N/A |
| I-33 | A | N/A |
| I-34 | A | N/A |
| I-35 | A | N/A |
| I-36 | A | N/A |
| I-37 | A | A |
| I-38 | A | A |
| I-39 | A | N/A |
| I-40 | A | A |
| I-41 | A | N/A |
| I-42 | A | N/A |
| I-43 | A | N/A |
| I-44 | C | N/A |
| I-45 | A | N/A |
| I-46 | A | N/A |
| I-47 | A | N/A |
| I-48 | A | N/A |
| I-49 | A | N/A |
| I-50 | A | N/A |
| I-51 | A | N/A |
| I-52 | A | N/A |
| I-53 | A | N/A |
| I-54 | B | N/A |
| I-55 | A | N/A |
| I-56 | A | N/A |
| I-57 | A | N/A |
| I-58 | A | N/A |
| I-59 | A | N/A |
| I-60 | C | N/A |
| I-61 | A | N/A |
| I-62 | A | N/A |
| I-63 | A | N/A |
| I-64 | A | N/A |
| I-65 | A | N/A |
| I-66 | A | N/A |
| I-67 | A | N/A |
| I-68 | C | N/A |
| I-69 | A | N/A |
| I-70 | A | N/A |
| I-71 | A | N/A |
| I-72 | A | N/A |
| I-73 | A | N/A |
| I-74 | A | N/A |
| I-75 | A | N/A |
| I-76 | A | N/A |
| I-78 | A | N/A |
| I-79 | A | N/A |
| I-80 | A | N/A |
| I-81 | A | N/A |
| I-82 | A | N/A |
| I-83 | A | N/A |
| I-84 | A | N/A |
| I-85 | A | N/A |
| I-86 | A | N/A |
| I-87 | A | N/A |
| I-88 | A | N/A |
| I-89 | A | N/A |
| I-90 | A | N/A |
| I-91 | A | N/A |
| I-92 | A | N/A |
| I-93 | A | N/A |
| I-94 | A | N/A |
| I-95 | A | N/A |
| I-96 | A | N/A |
| I-97 | A | N/A |
| I-98 | A | N/A |
| I-99 | A | N/A |
| I-100 | A | N/A |
| I-101 | A | N/A |
| I-102 | A | N/A |
| I-103 | A | N/A |
| I-104 | A | N/A |
| I-105 | B | N/A |
| I-106 | A | N/A |
| I-107 | A | N/A |
| I-108 | A | N/A |
| I-109 | C | N/A |
| I-110 | A | N/A |
| I-111 | A | N/A |
| I-112 | C | N/A |
| I-113 | A | N/A |
| I-114 | A | N/A |
| I-115 | A | N/A |
| I-116 | A | N/A |
| I-117 | A | N/A |
| I-118 | B | N/A |
| I-119 | A | N/A |
| I-120 | A | N/A |
| I-121 | A | N/A |
| I-122 | A | N/A |
| I-123 | A | N/A |
| I-124 | A | N/A |
| I-125 | A | N/A |
| I-126 | A | N/A |
| I-127 | B | N/A |
| I-128 | B | N/A |
| I-129 | A | N/A |
| I-130 | A | N/A |
| I-131 | A | N/A |
| I-132 | A | N/A |
| I-133 | A | N/A |
| I-134 | A | N/A |
| I-135 | A | N/A |
| I-136 | A | N/A |
| I-137 | A | N/A |
| I-138 | A | N/A |
| I-139 | A | N/A |
| I-140 | B | N/A |
| I-141 | A | N/A |
| I-142 | A | N/A |
| I-143 | A | N/A |
| I-144 | A | N/A |
| I-145 | A | N/A |
| I-146 | A | N/A |
| I-147 | C | N/A |
| I-148 | C | N/A |
| I-149 | A | N/A |
| I-150 | A | N/A |
| I-151 | A | N/A |
| I-152 | A | N/A |
| I-153 | A | N/A |
| I-154 | A | N/A |
| I-155 | A | N/A |
| I-156 | A | N/A |
| I-157 | A | N/A |
| I-158 | A | N/A |
| I-159 | A | N/A |
| I-160 | A | N/A |
| I-161 | C | N/A |
| I-162 | C | N/A |
| I-163 | A | N/A |
| I-164 | A | N/A |
| I-165 | C | N/A |
| I-166 | A | N/A |
| I-167 | C | N/A |
| I-168 | C | N/A |
| I-169 | c | N/A |
| I-170 | A | N/A |
| I-171 | A | N/A |
| I-172 | A | N/A |
| I-173 | B | N/A |
| I-174 | B | N/A |
| I-175 | A | N/A |
| I-176 | A | N/A |
| I-177 | A | N/A |
| I-178 | A | N/A |
| I-179 | B | N/A |
| I-180 | C | N/A |
| I-181 | C | N/A |
| I-182 | B | N/A |
| I-183 | C | N/A |
| I-184 | B | N/A |
| I-185 | A | N/A |
| I-186 | A | N/A |
| I-187 | A | N/A |
| I-188 | A | N/A |
| I-189 | A | N/A |
| I-190 | A | N/A |
| I-191 | C | N/A |
| I-194 | A | N/A |
| I-195 | B | N/A |
| I-196 | A | N/A |
| I-197 | A | N/A |
| I-198 | A | N/A |
| I-199 | B | N/A |
| I-200 | A | N/A |
| I-201 | B | N/A |
| I-202 | C | N/A |
| I-203 | B | N/A |
| I-204 | B | N/A |
| I-205 | A | N/A |
| I-206 | A | N/A |
| I-207 | A | N/A |
| I-208 | A | N/A |
| I-209 | B | N/A |
| I-210 | C | N/A |
| I-211 | A | N/A |
| I-212 | A | N/A |
| I-213 | A | N/A |
| I-214 | A | N/A |
| I-215 | A | N/A |
| I-216 | A | N/A |
| I-217 | B | N/A |
| I-218 | C | N/A |
| I-219 | A | N/A |
| I-220 | A | N/A |
| I-221 | B | N/A |
| I-222 | A | N/A |
| I-223 | B | N/A |
| I-224 | C | N/A |
| I-225 | C | N/A |
| I-227 | A | N/A |
| I-228 | B | N/A |
| I-229 | C | N/A |
| I-230 | C | N/A |
| I-231 | C | N/A |
| I-232 | C | N/A |
| I-233 | A | N/A |
| I-234 | C | N/A |
| I-235 | C | N/A |
| I-236 | A | N/A |

All of the U.S. patents, U.S. patent application publications, U.S. patent applications, foreign patents, foreign patent applications, and non-patent publications referred to in this specification are incorporated herein by reference in their entireties, including U.S. provisional application Nos. 62/694,958, filed Jul. 6, 2018, 62/831,029, filed Apr. 8, 2019, and 62/860,505, filed Jun. 12, 2019.

While we have described a number of embodiments of this invention, it is apparent that our basic examples may be altered to provide other embodiments that utilize the compounds and methods of this invention. Therefore, it will be appreciated that the scope of this invention is to be defined by the appended claims rather than by the specific embodiments that have been represented by way of example.

We claim:

1. A compound of Formula VI: 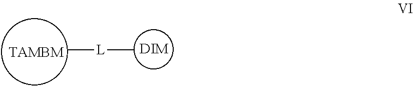
or a pharmaceutically acceptable salt thereof, wherein:
TAMBM is an TAM receptor kinase binding moiety;
L is a bivalent moiety that connects TAMBM to DIM; and
DIM is a degradation inducing moiety selected from LBM, a lysine mimetic, and hydrogen.

2. The compound of claim 1, wherein LBM is a ligase binding moiety, the LBM being a cereblon binding moiety, a VHL E3 ligase binding moiety, an IAP E3 ligase binding moiety, or an MDM2 E3 ligase binding moiety.

3. The compound of claim 2, wherein LBM is a cereblon binding moiety and said compound is of formula I-c-1: 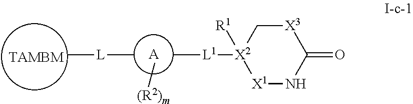
or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 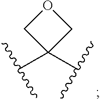 [SMILES: *C1(*)COC1]
X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a bi- or tricyclic ring selected from 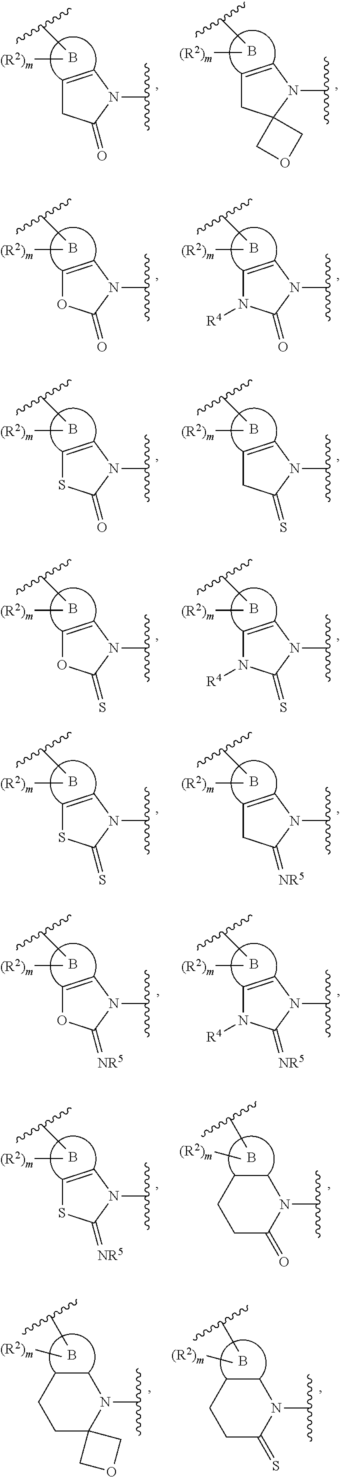 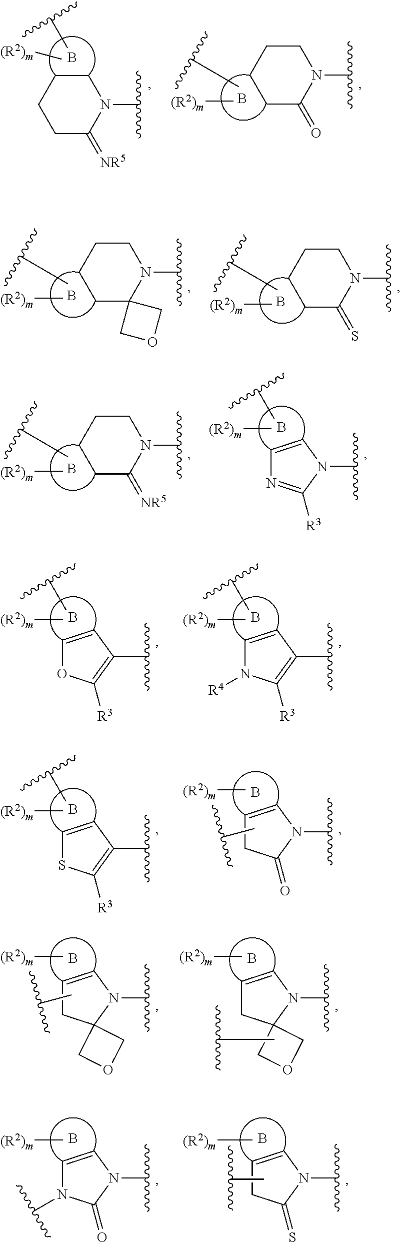 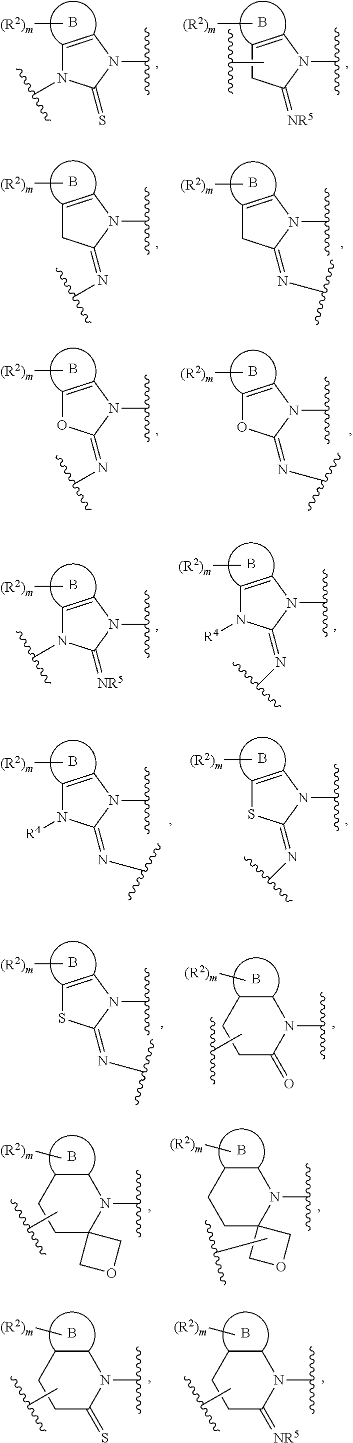 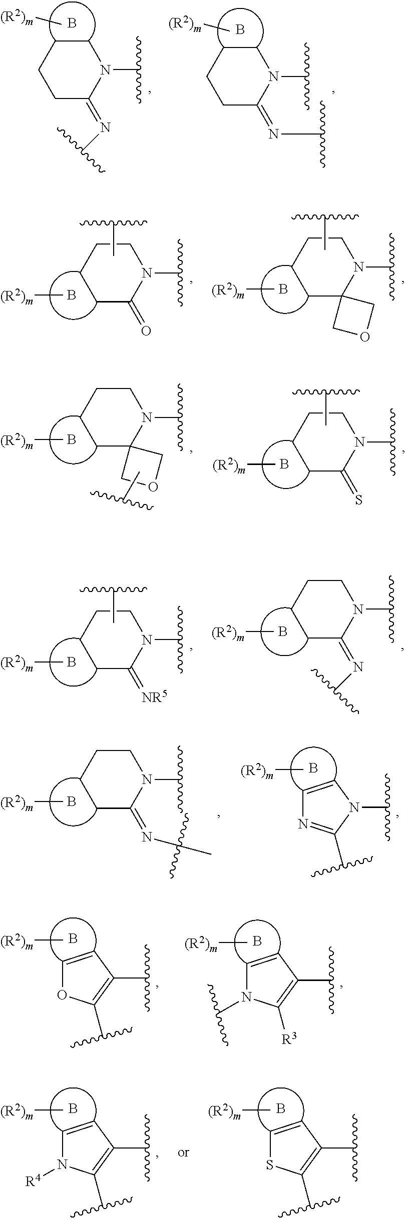
wherein
Ring B is a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-4 heteroatoms independently selected from nitrogen, oxygen or sulfur;
R³ is selected from hydrogen, halogen, —OR, —N(R)₂, or —SR;
each R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —(C)═CH—;
m is 0, 1, 2, 3 or4;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

4. The compound of claim 2, wherein LBM is a cereblon binding moiety and said compound is of formula I-d-1: 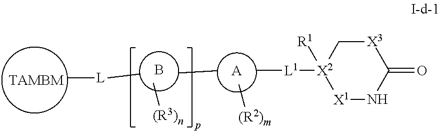
or a pharmaceutically acceptable salt thereof, wherein:
X′ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 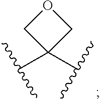 [SMILES: *C1(*)COC1]
X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a mono- or bicyclic ring selected from 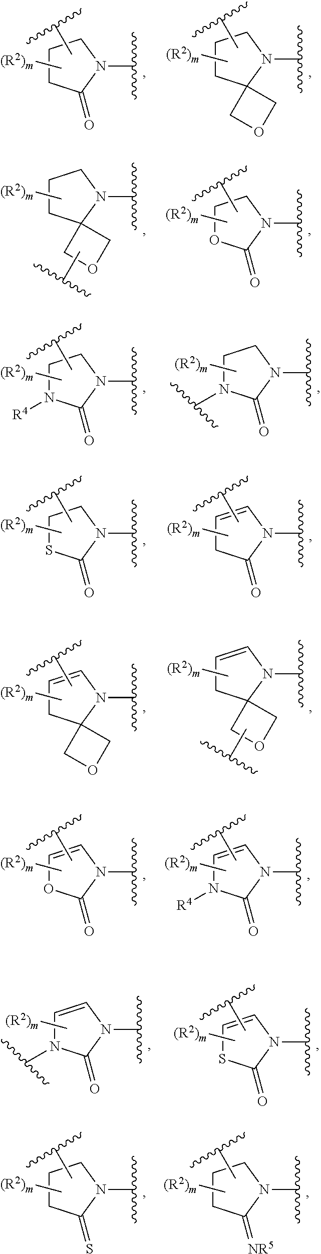 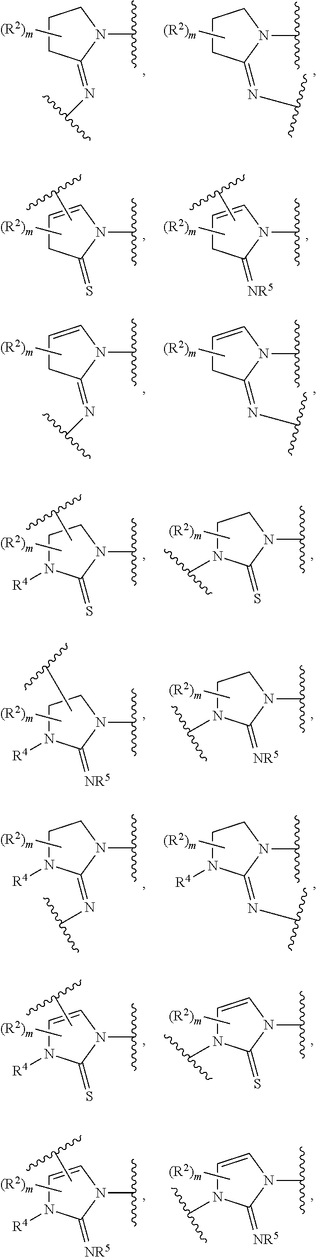 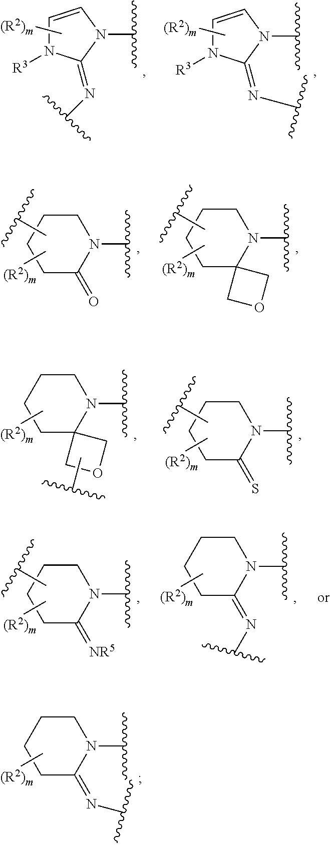
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—;
m is 0, 1, or 2;
n is 0, 1, 2, 3, or 4;
p is 0 or 1, wherein when p is 0, the bond connecting Ring A and Ring B is connected to 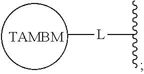
and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

5. The compound of claim 2, wherein LBM is a cereblon binding moiety and said compound is of formula I-e-1: 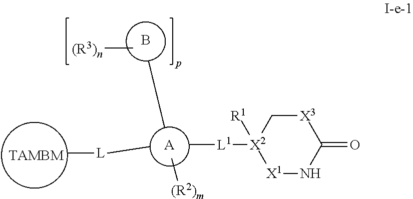
or a pharmaceutically acceptable salt thereof, wherein L and TAMBM are as defined above and described in embodiments herein, and wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 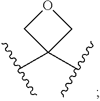 [SMILES: *C1(*)COC1]
X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R² is independently hydrogen, deuterium, —R⁶, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
Ring A is a mono- or bicyclic ring selected from 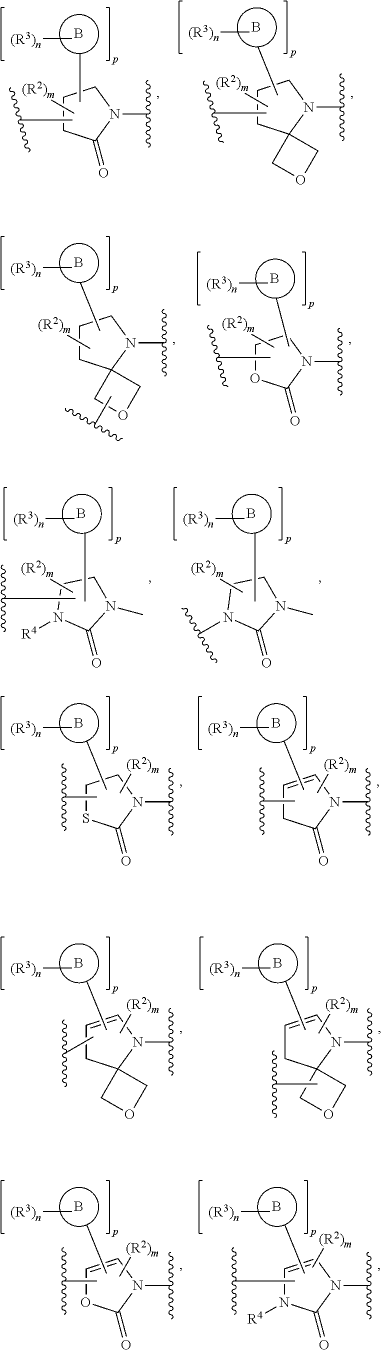 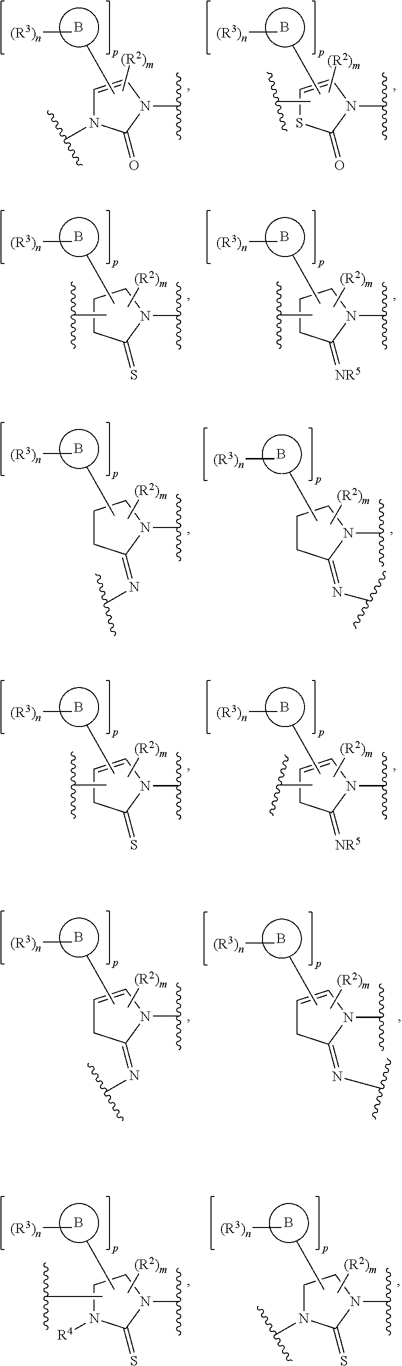 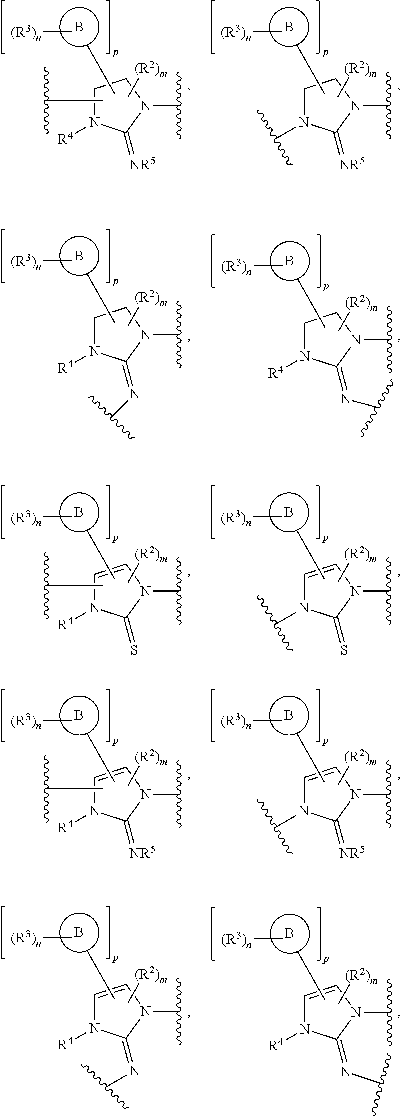 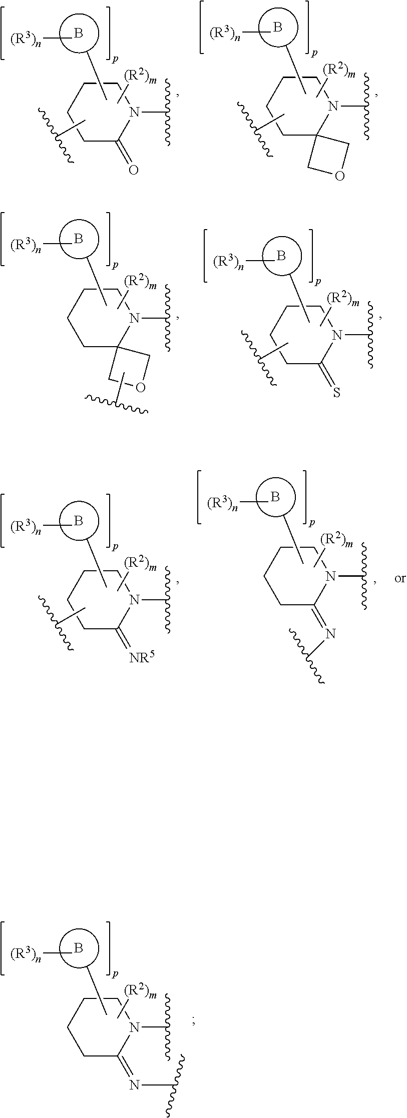
Ring B is selected from a 6-membered aryl containing 0-2 nitrogen atoms or a 5-membered heteroaryl with 1-3 heteroatoms independently selected from nitrogen, oxygen or sulfur;
each of R³ and R⁴ is independently hydrogen, —R⁶, halogen, —CN, —NO₂, —OR, —SR, —NR₂, —S(O)₂R, —S(O)₂NR₂, —S(O)R, —C(O)R, —C(O)OR, —C(O)NR₂, —C(O)N(R)OR, —OC(O)R, —OC(O)NR₂, —N(R)C(O)OR, —N(R)C(O)R, —N(R)C(O)NR₂, or —N(R)S(O)₂R;
R⁵ is hydrogen, C₁₋₄ aliphatic, or —CN;
each R⁶ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—;
m is 0, 1, or 2;
n is 0, 1, 2, 3, or 4;
p is 0 or 1; and
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are optionally taken together with their intervening atoms to form a 4-7 membered saturated, partially unsaturated, or heteroaryl ring having 0-3 heteroatoms, in addition to the nitrogen, independently selected from nitrogen, oxygen, and sulfur.

6. The compound of claim 2, wherein LBM is a cereblon binding moiety and said compound is of formula II-e: 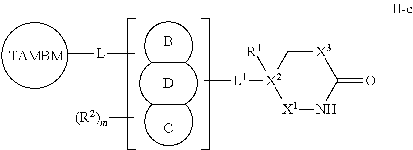
or a pharmaceutically acceptable salt thereof, wherein:
X¹ is a bivalent moiety selected from a covalent bond, —CH₂—, —CHCF₃—, —SO₂—, —S(O)—, —P(O)R—, —P(O)OR—, —P(O)NR₂—, —C(O)—, —C(S)—, or 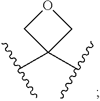 [SMILES: *C1(*)COC1]
X² is a carbon atom or silicon atom;
X³ is a bivalent moiety selected from —CR₂—, —NR—, —O—, —S—, or —Si(R₂)—;
R¹ is hydrogen, deuterium, halogen, —CN, —OR, —SR, —S(O)R, —S(O)₂R, —N(R)₂, —P(O)(OR)₂, —P(O)(NR₂)OR, —P(O)(NR₂)₂, —Si(OH)₂R, —Si(OH)(R)₂, —Si(R)₃, or an optionally substituted C₁₋₄ aliphatic;
each R is independently hydrogen, or an optionally substituted group selected from C₁₋₆ aliphatic, phenyl, a 4-7 membered saturated or partially unsaturated heterocyclic having 1-2 heteroatoms independently selected from nitrogen, oxygen, and sulfur, and a 5-6 membered heteroaryl ring having 1-4 heteroatoms independently selected from nitrogen, oxygen, and sulfur, or:
two R groups on the same nitrogen are taken together with their intervening atoms to form a 4-7 membered saturated, partially unsaturated, or heteroaryl ring having 0-3 heteroatoms, in addition to the nitrogen, independently selected from nitrogen, oxygen, and sulfur;
each R² is independently hydrogen, deuterium, —R³, halogen, —CN, —NO₂, —OR, —SR, —N(R)₂, —Si(R)₃, —S(O)₂R, —S(O)₂N(R)₂, —S(O)R, —C(O)R, —C(O)OR, —C(O)N(R)₂, —C(O)N(R)OR, —C(R)₂N(R)C(O)R, —C(R)₂N(R)C(O)N(R)₂, —OC(O)R, —OC(O)N(R)₂, —OP(O)R₂, —OP(O)(OR)₂, —OP(O)(OR)(NR₂), —OP(O)(NR₂)₂—, —N(R)C(O)OR, —N(R)C(O)R, —N(R)C(O)N(R)₂, —N(R)S(O)₂R, —NP(O)R₂, —N(R)P(O)(OR)₂, —N(R)P(O)(OR)(NR₂), —N(R)P(O)(NR₂)₂, or —N(R)S(O)₂R;
each R³ is independently an optionally substituted group selected from C₁₋₆ aliphatic, phenyl, a 4-7 membered saturated or partially unsaturated heterocyclic ring having 1-2 heteroatoms independently selected from nitrogen, oxygen, and sulfur, and a 5-6 membered heteroaryl ring having 1-4 heteroatoms independently selected from nitrogen, oxygen, and sulfur;
each of Ring B, Ring D, and Ring C is independently a fused ring selected from 6-membered aryl, 6-membered heteroaryl containing 1-4 heteroatoms independently selected from nitrogen, oxygen, or sulfur, 5 to 7-membered saturated or partially unsaturated carbocyclyl, 5 to 7-membered saturated or partially unsaturated heterocyclyl ring with 1-3 heteroatoms independently selected from boron, nitrogen, oxygen, silicon, or sulfur, or 5-membered heteroaryl with 1-4 heteroatoms independently selected from nitrogen, oxygen or sulfur;
L¹ is a covalent bond or a C₁₋₃ bivalent straight or branched saturated or unsaturated hydrocarbon chain wherein 1-2 methylene units of the chain are independently and optionally replaced with —O—, —C(O)—, —C(S)—, —C(R)₂—, —CH(R)—, —C(F)₂—, —N(R)—, —S(O)₂— or —CR═CR—; and
m is 0, 1, 2, 3, 4, 5, 6, 7, 8, 9, 10, 11, 12, 13, 14, 15, or 16.

7. The compound of claim 1, wherein LBM is a cereblon binding moiety and said compound is selected from any of the following formulae:
(i) 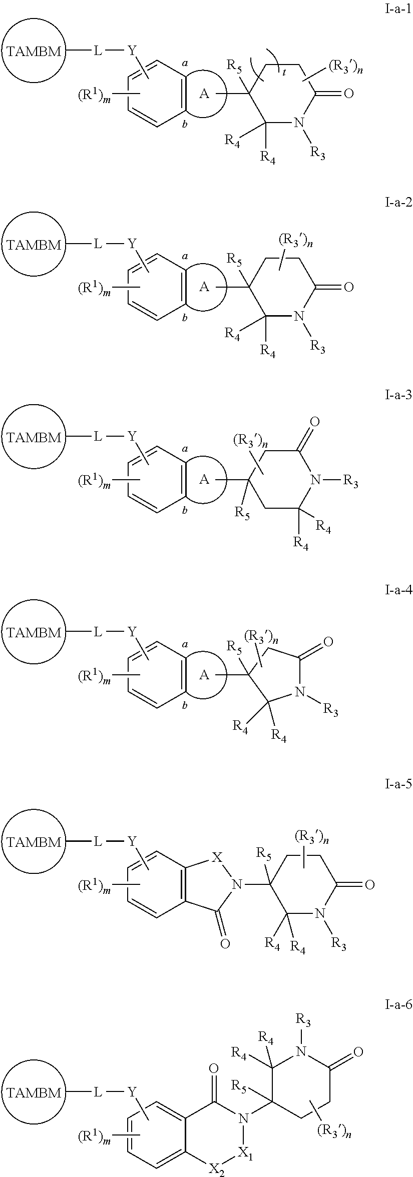 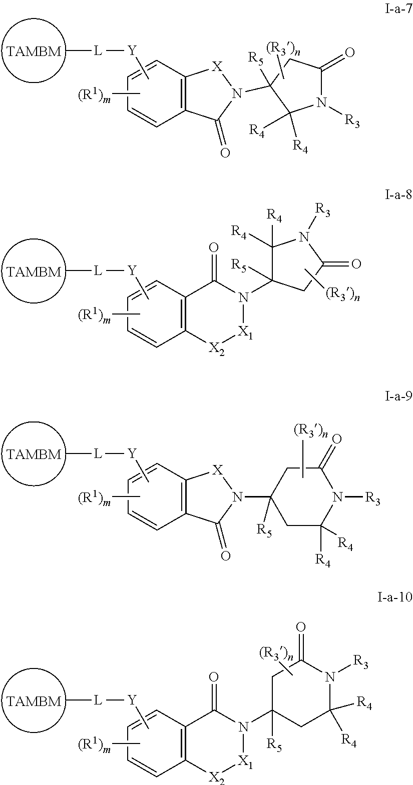
or a pharmaceutically acceptable salt thereof, wherein each of the variables 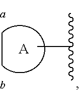
X, X₁, X₂, Y, R₁, R₃, R₃′, R₄, R₅, t, m and n is as defined and described in WO 2017/007612 and US2018/0134684;
(ii) 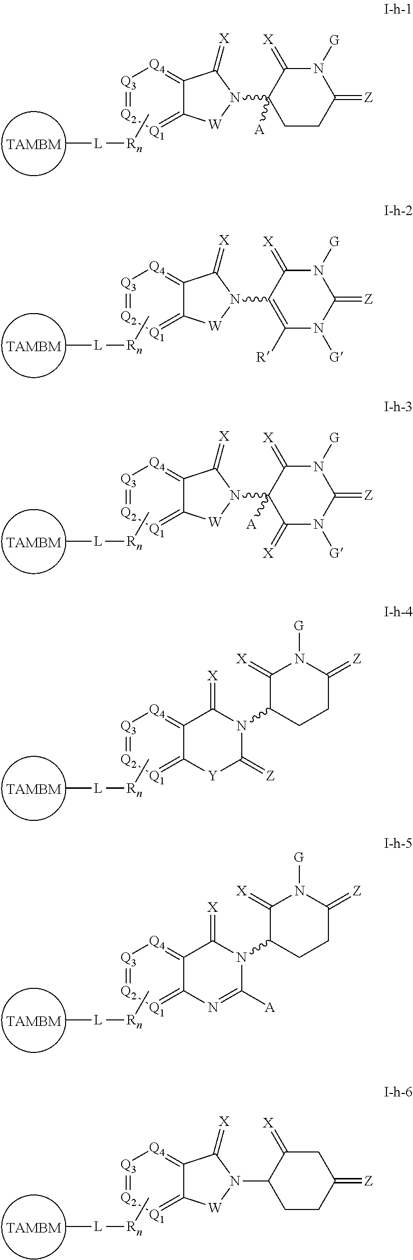
or a pharmaceutically acceptable salt thereof, wherein each of the variables A, G, G′, Q₁, Q₂, Q₃, Q₄, R, R′, W, X, Z, and n is as defined and described in WO 2016/197114 and US2018/0147202;
(iii) 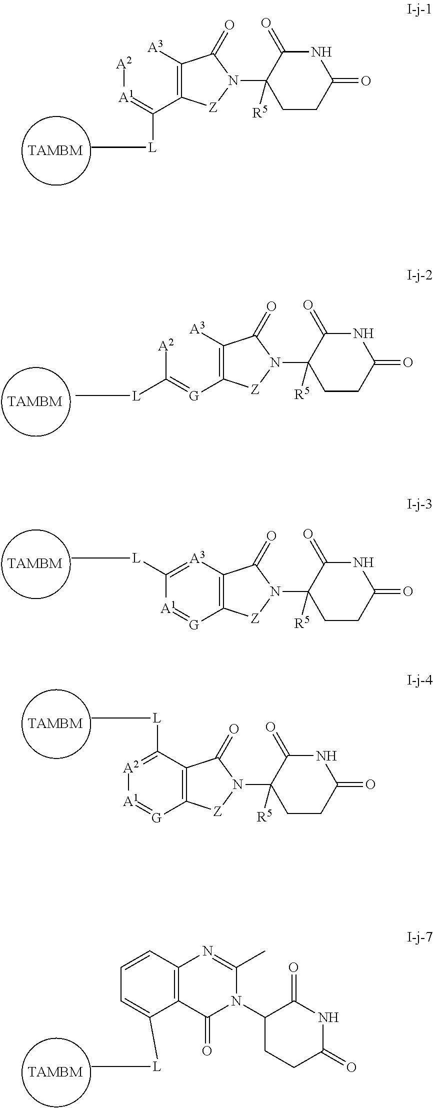
or a pharmaceutically acceptable salt thereof, wherein each of the variables A¹, A², A³, R⁵, G and Z is as defined and described in WO 2017/176958 and US2019/0119289;
(iv) 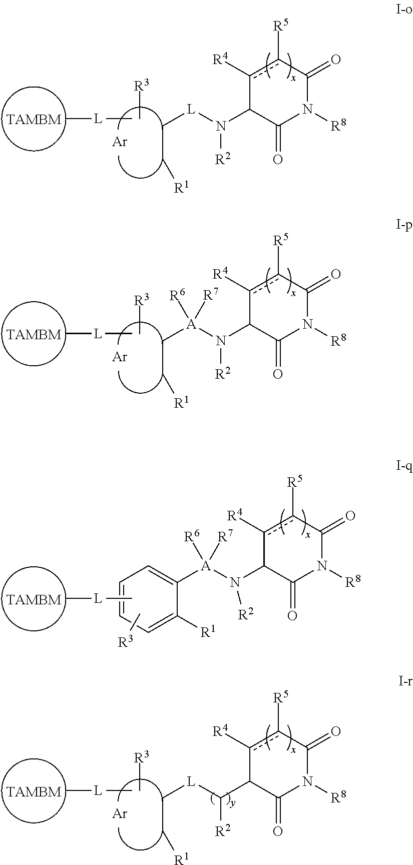 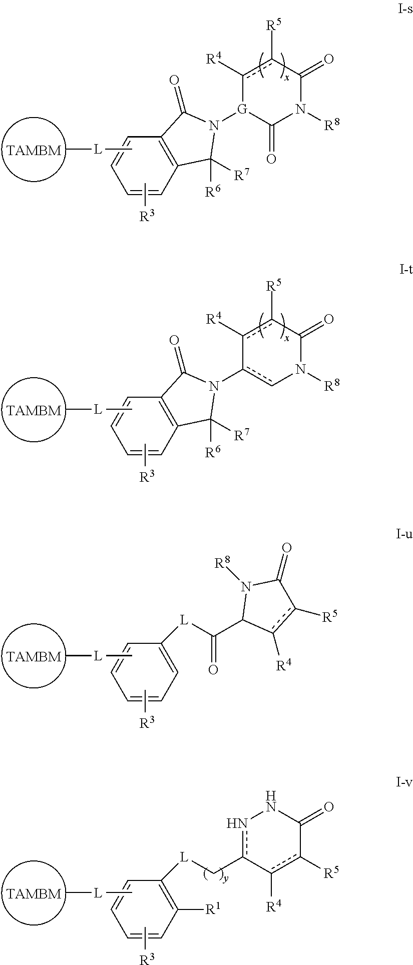
or a pharmaceutically acceptable salt thereof, wherein each of the variables variables G, Ar, R¹, R², R³, R⁴, R⁵, R⁶, R⁷, R⁸, A, L, x, y, and the bond [symbol] is as described and defined in WO 2017/161119;
(v) 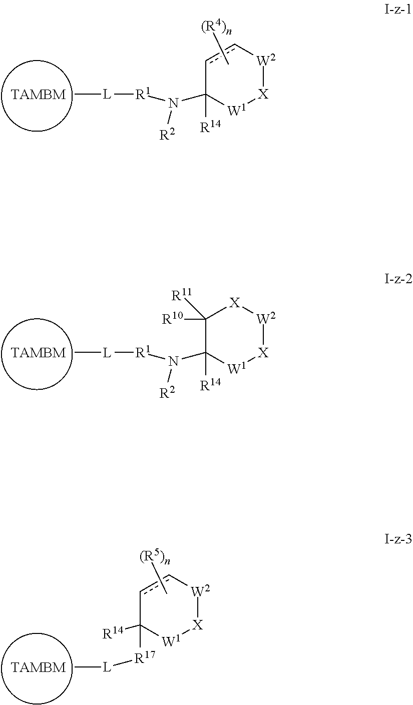
or a pharmaceutically acceptable salt thereof, wherein each of the variables R¹, R², R⁴, R⁵, R¹⁰, R¹¹, R¹⁴, R¹⁷, W¹, W², X and n is as defined in WO 2017/197051 and US 2019/0076539; or
(vi) 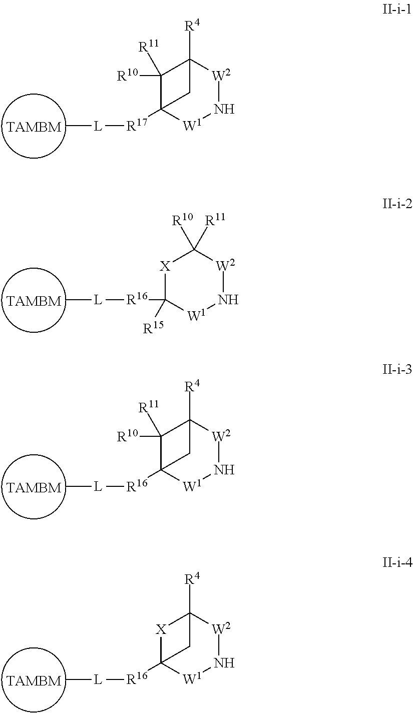
or a pharmaceutically acceptable salt thereof, wherein each of the variables R⁴, R¹⁰, R¹¹, R¹⁵, R¹⁶, R¹⁷, W¹, W², and X is as defined in WO 2019/099868.

8. The compound according to claim 2, wherein cereblon ligase binding moiety is selected from:

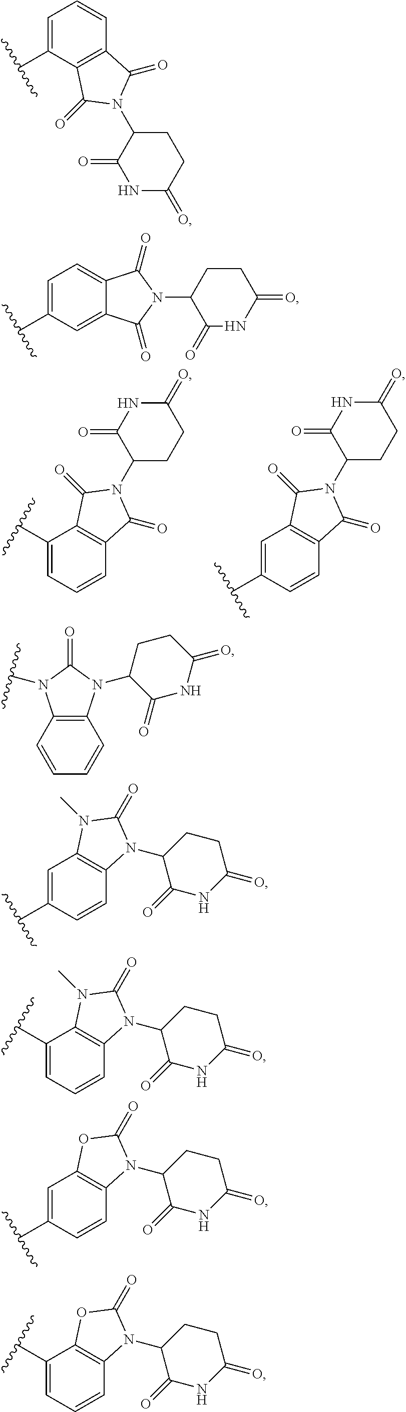

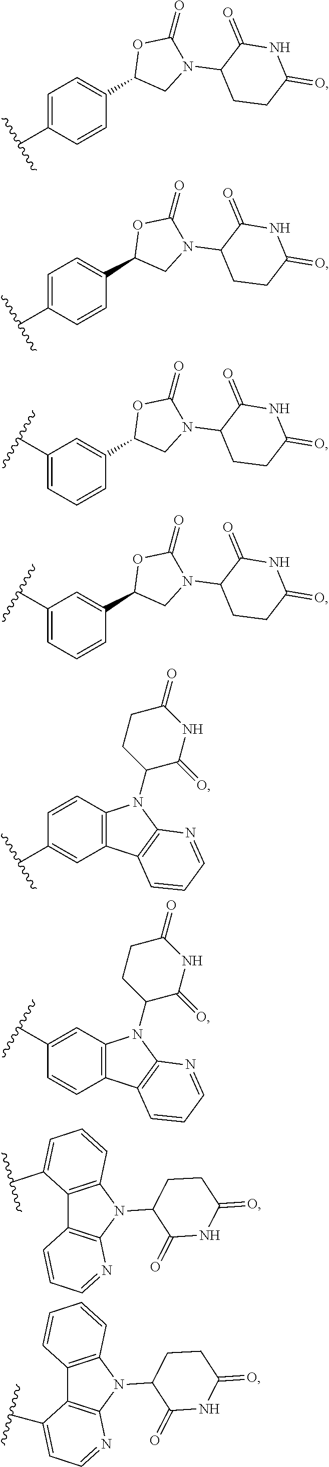

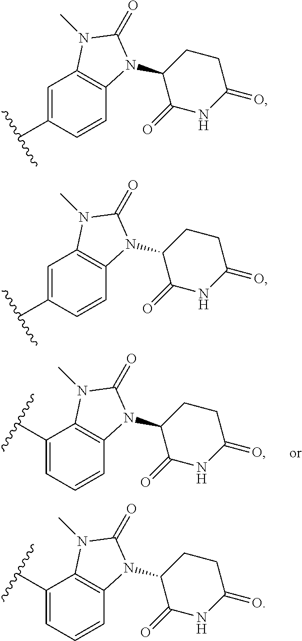

9. The compound of claim 2, wherein LBM is a VHL E3 ligase binding moiety and said compound is selected from any of the following formulae:
(i) 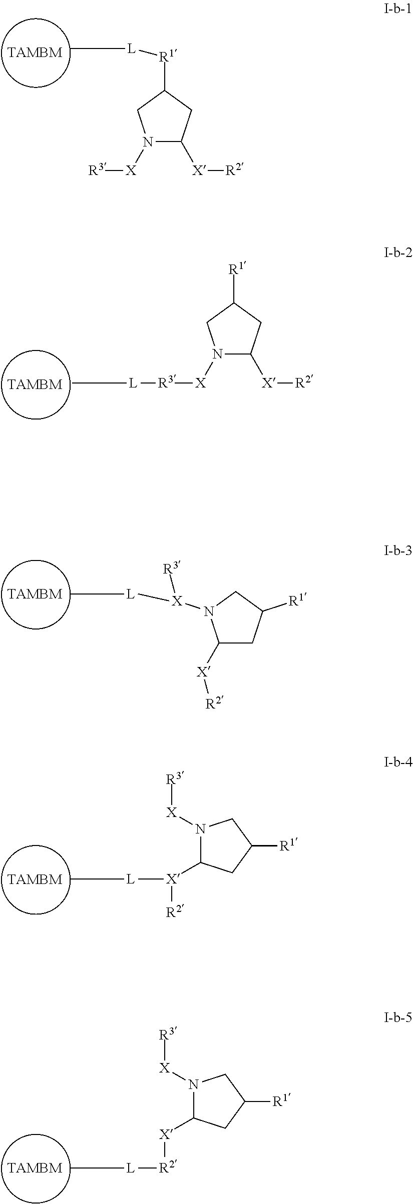
or a pharmaceutically acceptable salt thereof, wherein each of the variables R^{1′}, R^{2′}, R^{3′}, X, and X′ is as defined and described in WO 2013/106643 and US 2014/0356322;
(ii) 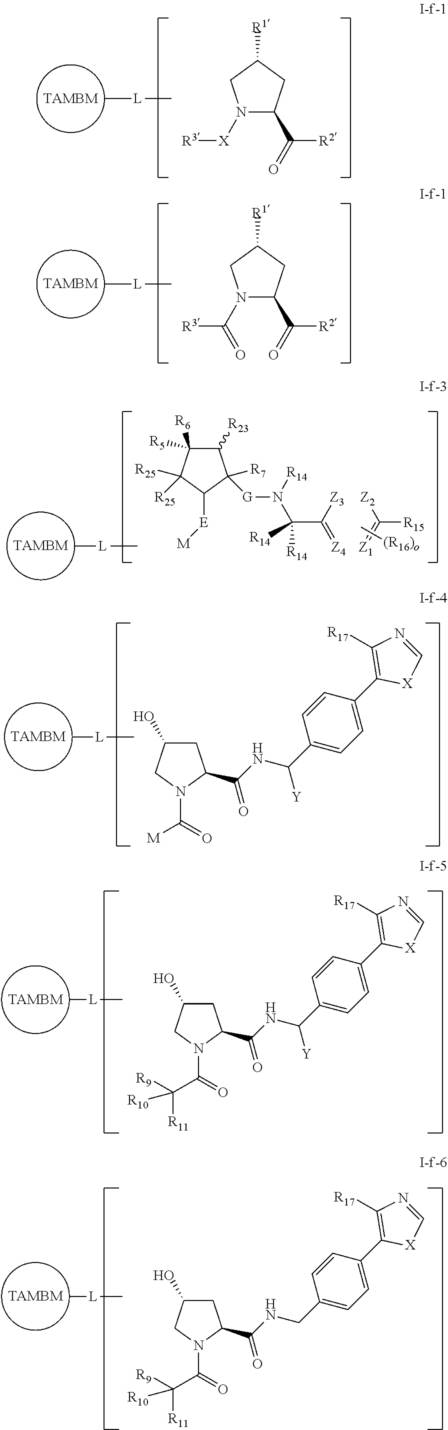
or a pharmaceutically acceptable salt thereof, wherein each of the variables R^{1′}, R^{2′}, R^{3′}, R₅, R₆, R₇, R₉, R₁₀, R₁₁, R₁₄, R₁₅, R₁₆, R₁₇, R₂₃, R₂₅, E, G, M, X, X′, Y, Z₁, Z₂, Z₃, Z₄, and o is as defined and described in WO 2016/149668 and US 2016/0272639;
(iii) 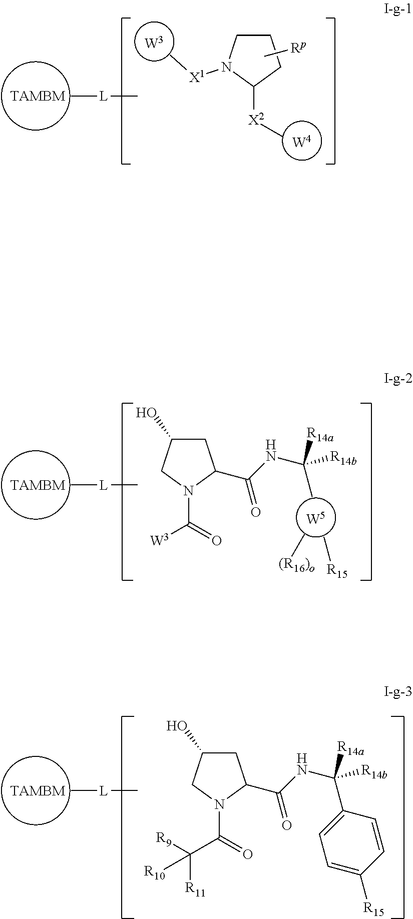
or a pharmaceutically acceptable salt thereof, wherein each of the variables R^{p}, R₉, R₁₀, R₁₁, R₁₄ₐ, R_{14b}, R₁₅, R₁₆, W³, W⁴, W⁵, X¹, X², and o is as defined and described in WO 2016/118666 and US 2016/0214972;
(iv) 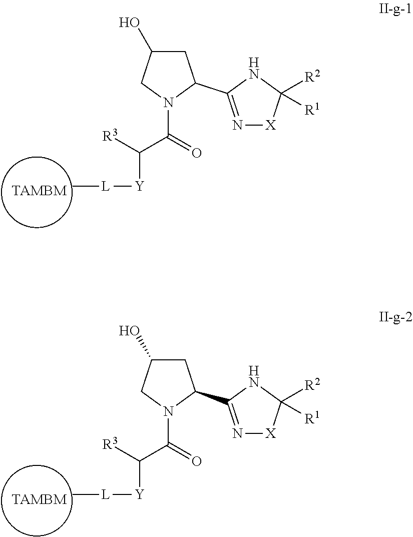
or a pharmaceutically acceptable salt thereof, wherein each of the variables R¹, R², R³, X, and Y is as defined and described in WO 2019/084026; or
(v) 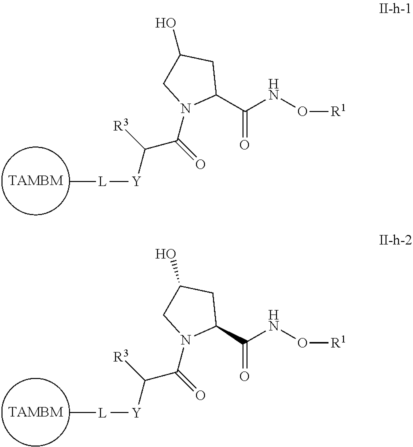
or a pharmaceutically acceptable salt thereof, wherein each of the variables R¹, R³, and Y is as defined and described in WO 2019/084030.

9. The compound according to claim 2, wherein VHL E3 ligase binding moiety is selected from:

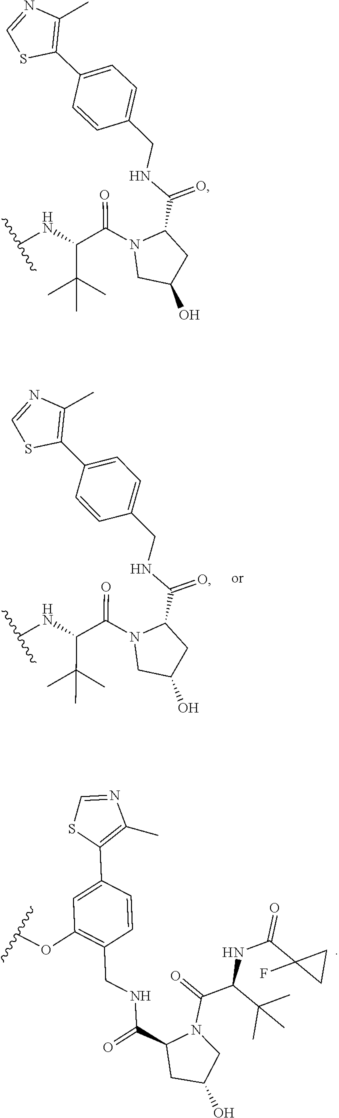 [SMILES: *N[C@H](C(=O)N1C[C@H](O)C[C@H]1C(=O)NCc1ccc(-c2scnc2C)cc1)C(C)(C)C ; *N[C@H](C(=O)N1C[C@@H](O)C[C@H]1C(=O)NCc1ccc(-c2scnc2C)cc1)C(C)(C)C ; "or" ; *Oc1cc(-c2scnc2C)ccc1CNC(=O)[C@@H]1C[C@@H](O)CN1C(=O)[C@@H](NC(=O)C1(F)CC1)C(C)(C)C]

10. The compound of claim 2, wherein LBM is a MDM2 E3 ligase binding moiety and said compound is selected from any of the following formulae: 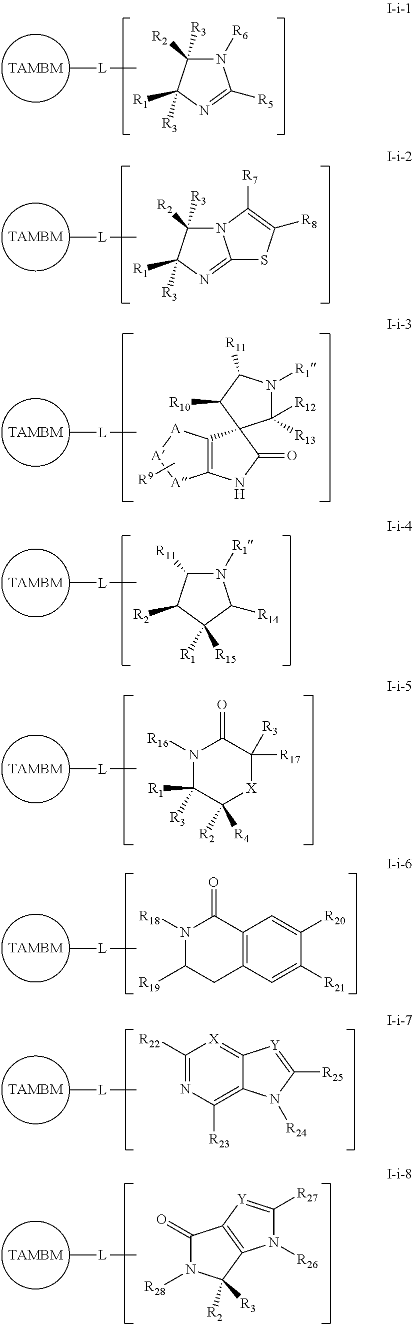 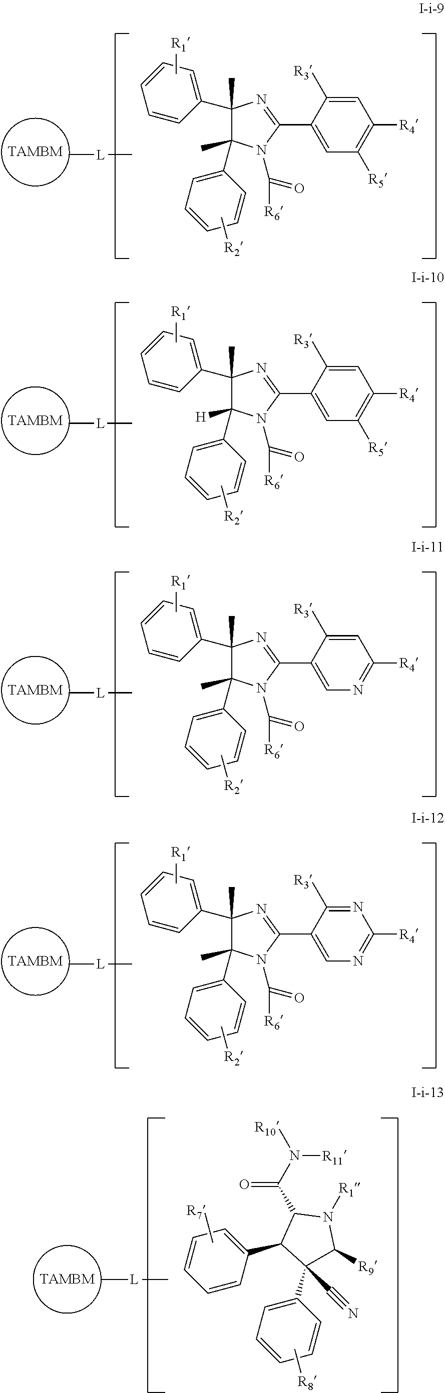 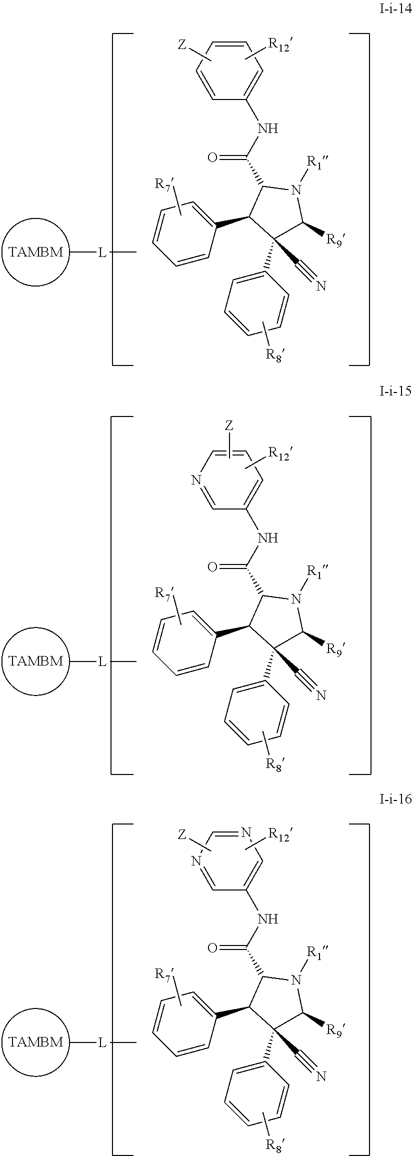 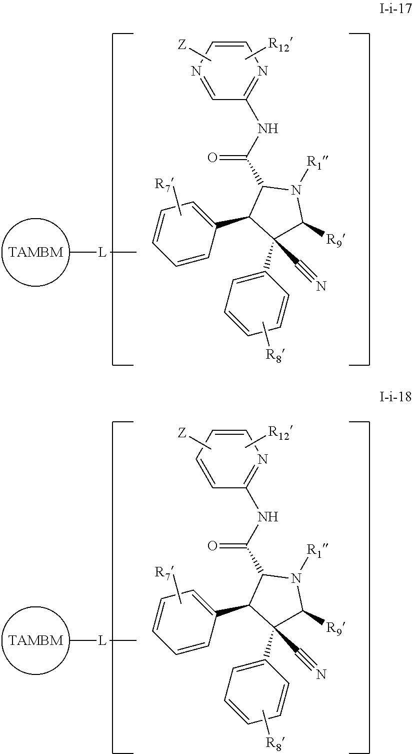
or a pharmaceutically acceptable salt thereof, wherein each of the variables R₁, R₂, R₃, R₄, R₅, R₆, R₇, R₈, R₉, R₁₀, R₁₁, R₁₂, R₁₃, R₁₄, R₁₅, R₁₆, R₁₇, R₁₈, R₁₉, R₂₀, R₂₁, R₂₂, R₂₃, R₂₄, R₂₅, R₂₆, R₂₇, R₂₈, R_{′}, R_{2′}, R_{3′}, R_{4′}, R_{5′}, R_{6′}, R_{7′}, R_{8′}, R_{9′}, R_{10′}, R_{11′}, R_{12′}, R_{12′}, R_{1″}, A, A′, A″, X, Y, and Z is as defined and described in WO 2017/011371 and US 2017/0008904.

11. The compound according to claim 2, wherein MDM2 E3 ligase binding moiety is selected from:

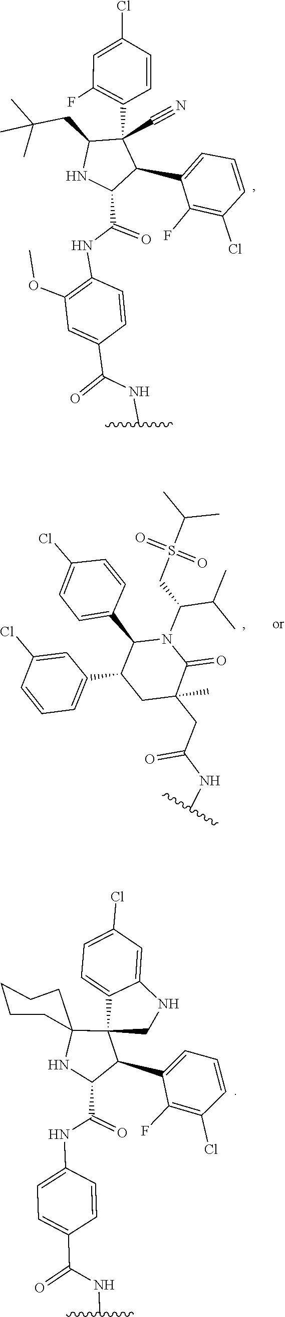 [SMILES: *NC(=O)c1ccc(NC(=O)[C@@H]2N[C@@H](CC(C)(C)C)[C@](C#N)(c3ccc(Cl)cc3F)[C@H]2c2cccc(Cl)c2F)c(OC)c1 ; *NC(=O)C[C@@]1(C)C[C@H](c2cccc(Cl)c2)[C@@H](c2ccc(Cl)cc2)N([C@H](CS(=O)(=O)C(C)C)C(C)C)C1=O ; *NC(=O)c1ccc(NC(=O)[C@@H]2NC3(CCCCC3)[C@@]3(CNc4cc(Cl)ccc43)[C@H]2c2cccc(Cl)c2F)cc1]

12. The compound of claim 2, wherein LBM is a TAP E3 ligase binding moiety and said compound is selected from any of the following formulae:
(i) 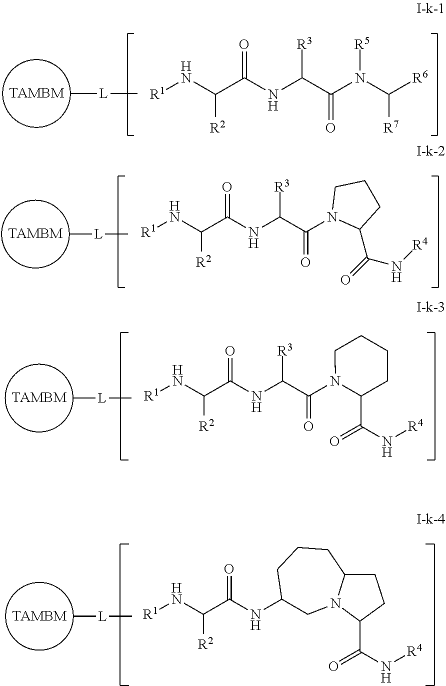
or a pharmaceutically acceptable salt thereof, wherein each of the variables R¹, R², R³, R⁴, R⁵, R⁶, and R⁷, is as defined and described in WO 2017/011590 and US 2017/0037004; or
(ii) 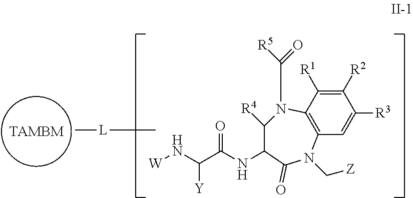
or a pharmaceutically acceptable salt thereof, wherein each of the variables W, Y, Z, R¹, R², R³, R⁴, and R⁵ is as described and defined in WO 2014/044622, US 2015/0225449, WO 2015/071393, and US 2016/0272596.

13. The compound according to claim 2, wherein TAP E3 ligase binding moiety is selected from:

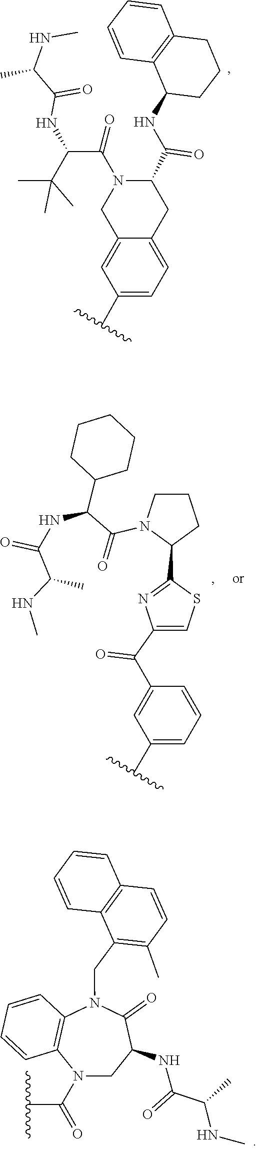 [SMILES: *c1ccc2c(c1)CN(C(=O)[C@@H](NC(=O)[C@H](C)NC)C(C)(C)C)[C@H](C(=O)N[C@@H]1CCCc3ccccc31)C2 ; *c1cccc(C(=O)c2csc([C@@H]3CCCN3C(=O)[C@@H](NC(=O)[C@H](C)NC)C3CCCCC3)n2)c1 ; *C(=O)N1C[C@H](NC(=O)[C@H](C)NC)C(=O)N(Cc2c(C)ccc3ccccc23)c2ccccc21]

14. The compound of claim 1, wherein DIM is a lysine mimetic and said compound is selected from any one of the following formulae:
(i) 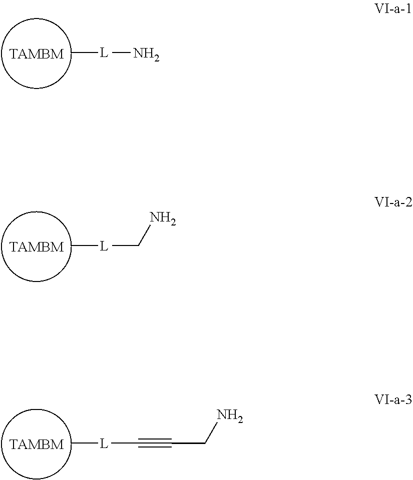
or a pharmaceutically acceptable salt thereof; or
(ii) 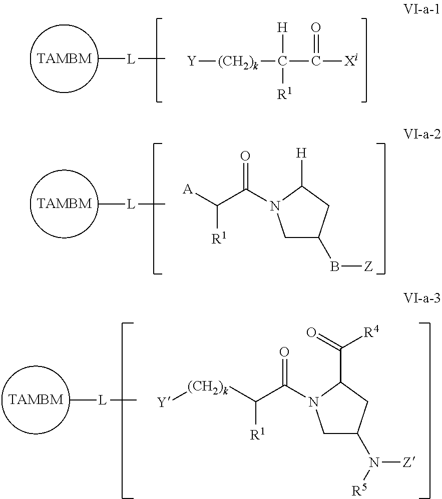
or a pharmaceutically acceptable salt thereof, wherein each of the variables R¹, R⁴, R⁵, A, B, E, Y, Y′, Z, Z′, and k are as defined and described in U.S. Pat. No. 7,622,496.

15. The compound of claim 1, wherein DIM is a hydrogen atom and said compound is formula VI-c: 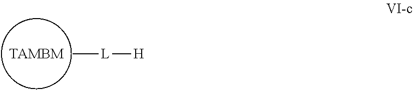
or a pharmaceutically acceptable salt thereof.

16. The compound of claim 1, wherein said compound is selected from any one of the following formulae:
(i) 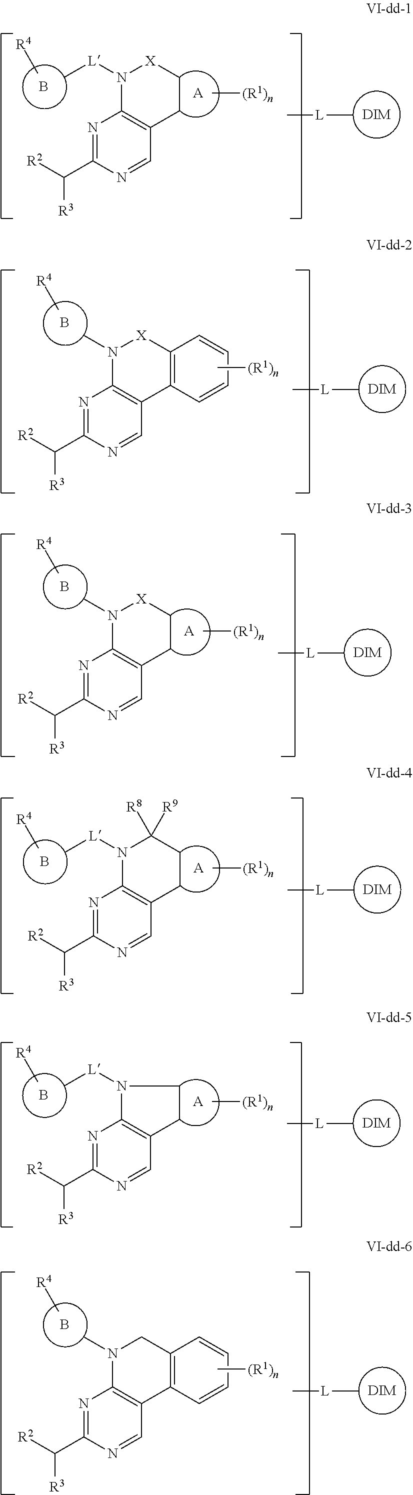 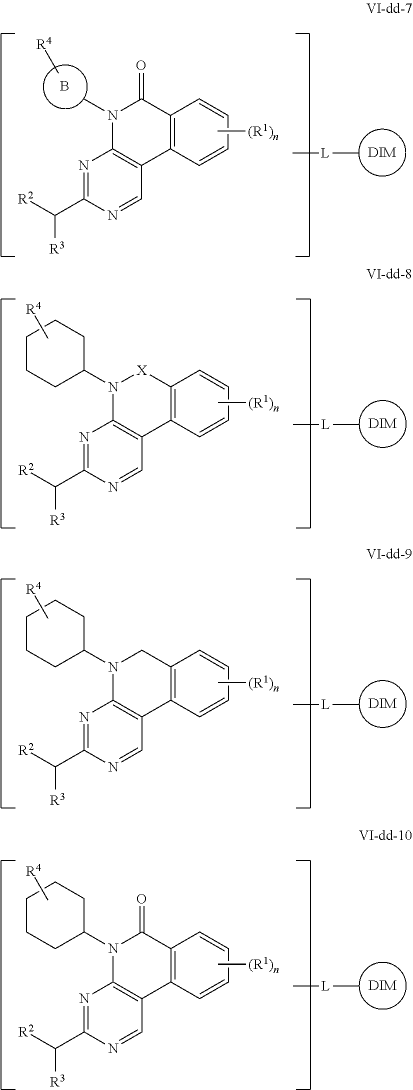
or a pharmaceutically acceptable salt thereof, wherein each of the variables, wherein each of Ring A, Ring B, R¹, R², R³, R⁴, R⁸, R⁹, L′, and n are as defined and described in U.S. pat. No. 9,840,503 and WO 2016/183071;
(ii) 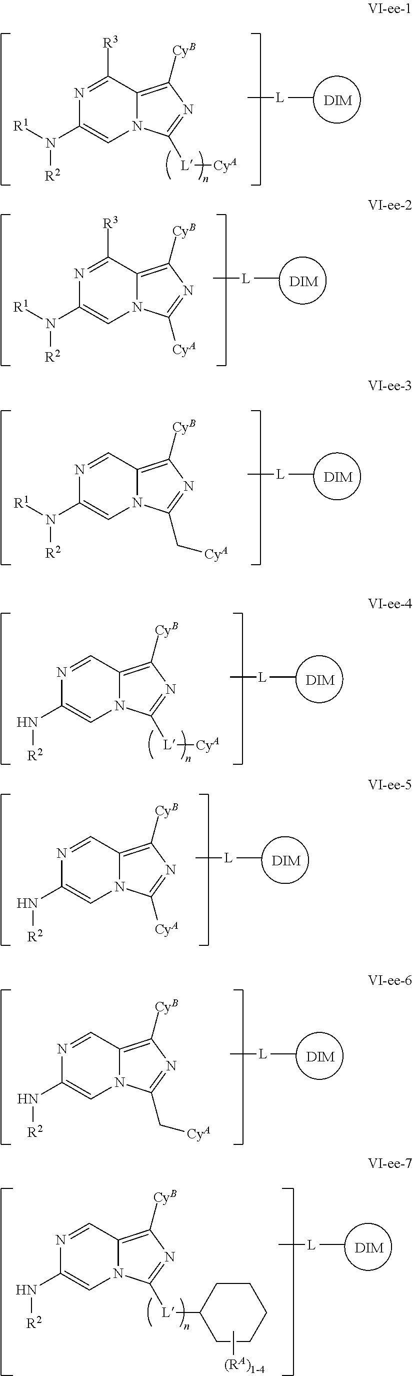 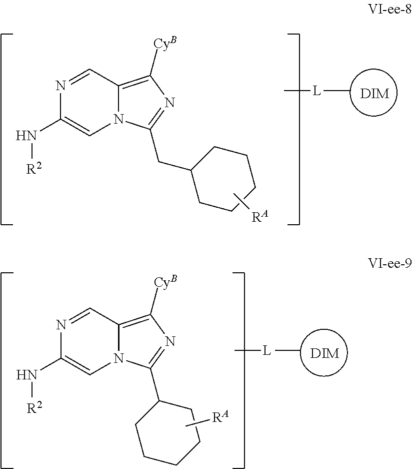
or a pharmaceutically acceptable salt thereof, wherein each of the variables wherein, each of Cy^{A}, Cy^{B}, L′, R¹, R², R³, R^{A}, and n are as defined and described in U.S. Pat. No. 9,708,333, US 2018/0009815, and WO 2017/027717;
(iii) 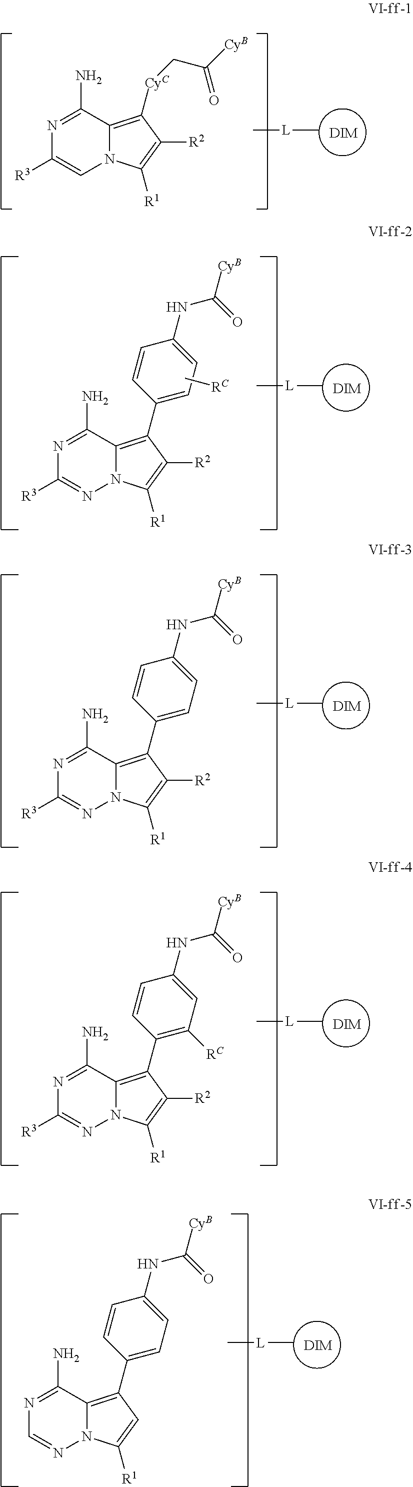 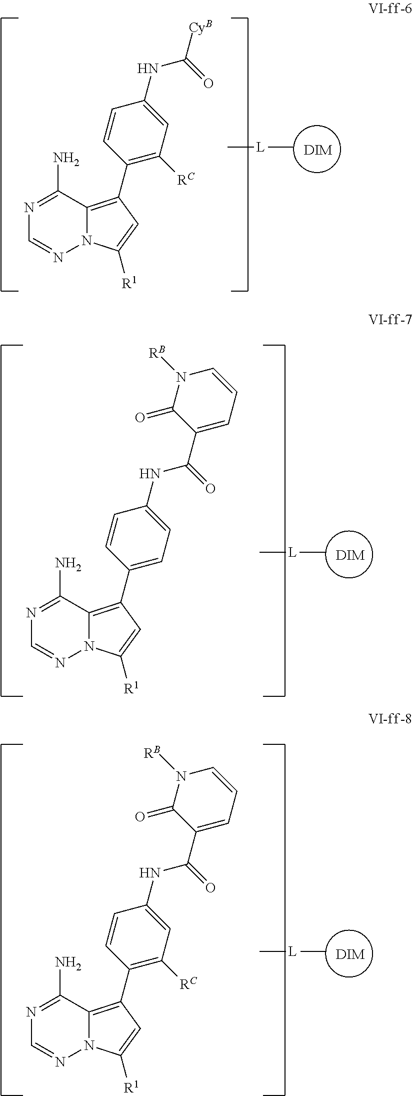 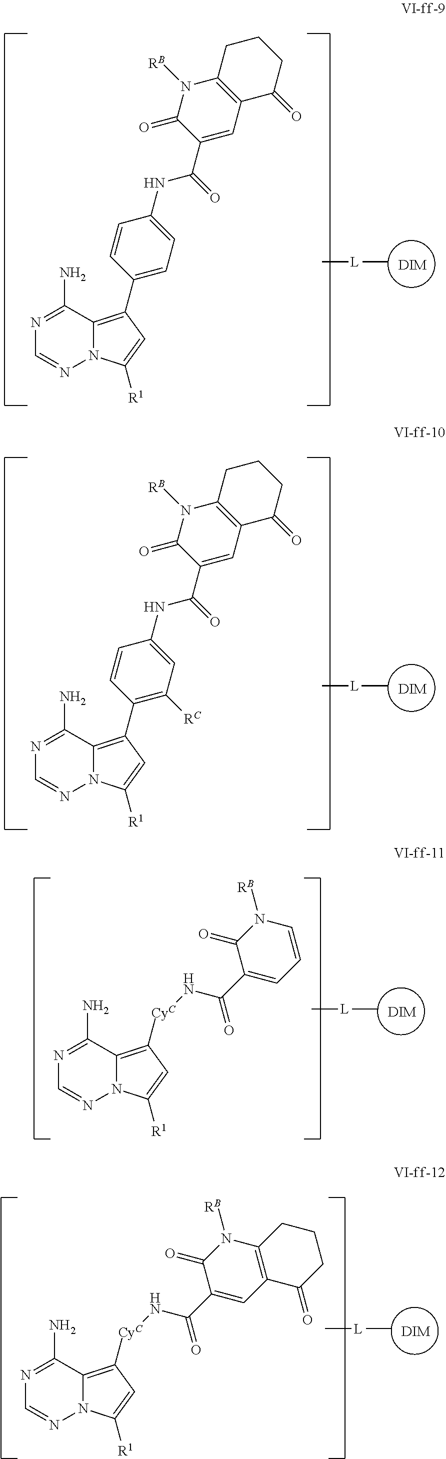 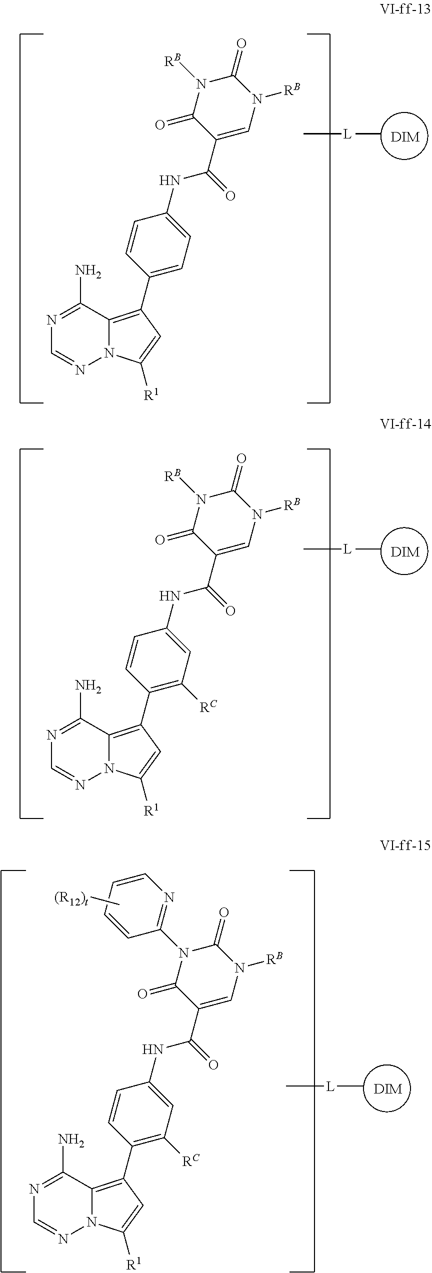 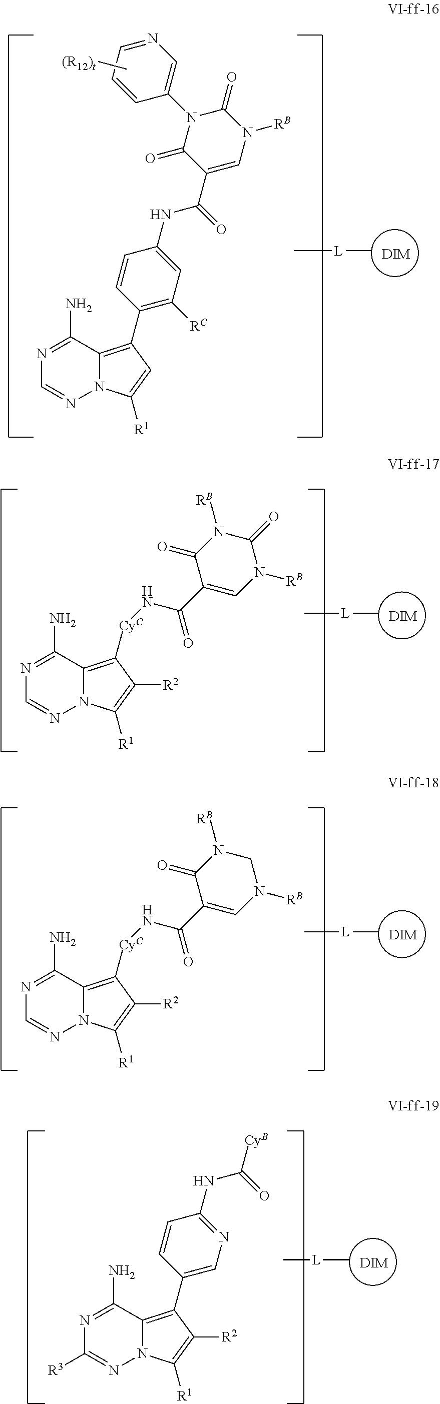 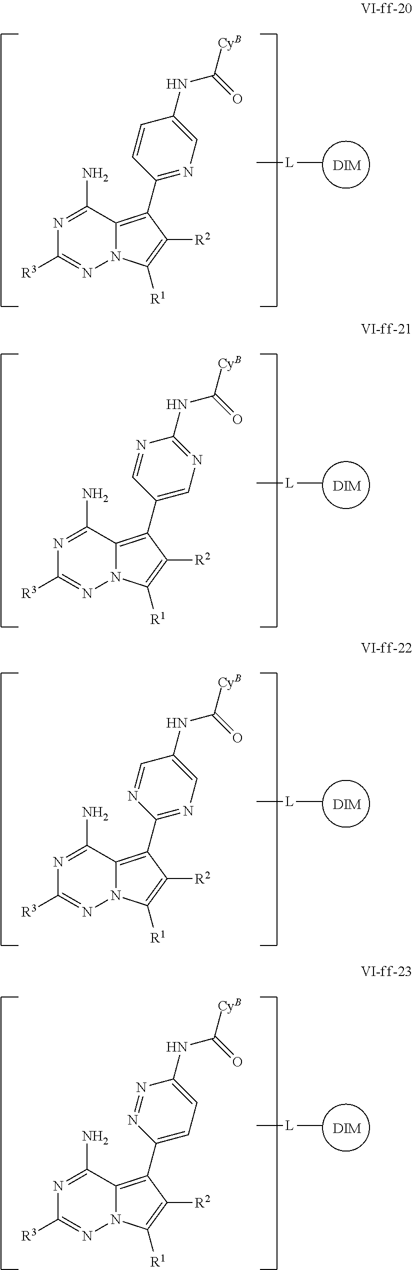 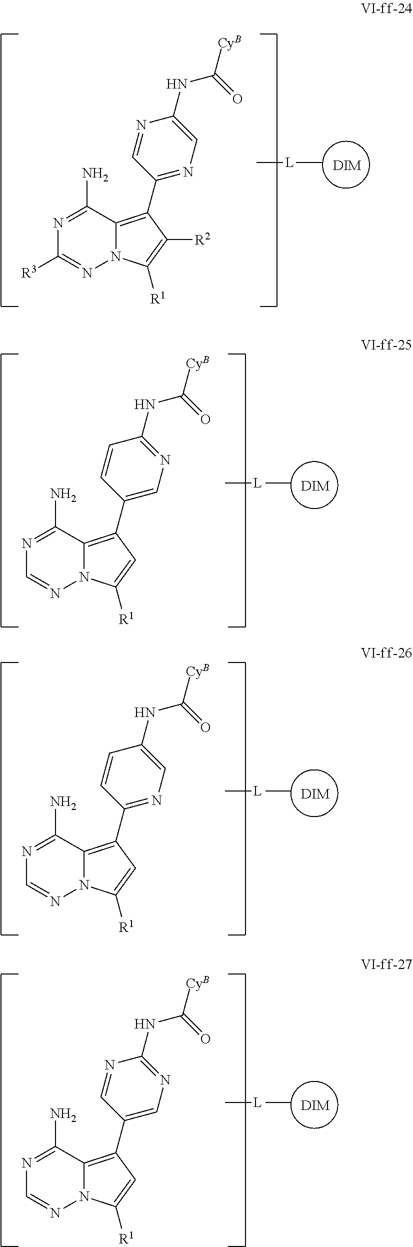 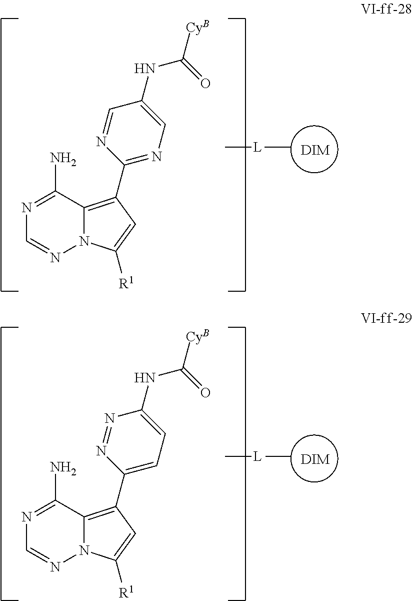
or a pharmaceutically acceptable salt thereof, wherein each of the variables Cy^{B}, Cy^{C}, R¹, R², R³, R^{B}, R^{C}, R₁₂, t, are as defined and described in US 2017/0275290 and U.S. Pat. No. 9,981,975;
(iv) 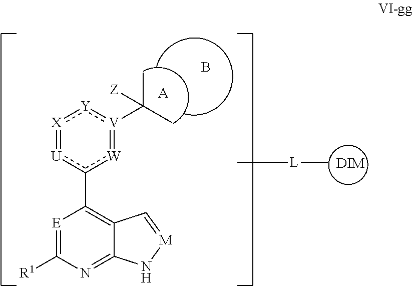
or a pharmaceutically acceptable salt thereof, wherein each of the variables Ring A, Ring B, R¹, E, N, U, V, W, X, Y, Z, M, are as defined and described in WO 2017/035366 and US 2017/0057965;
(v) 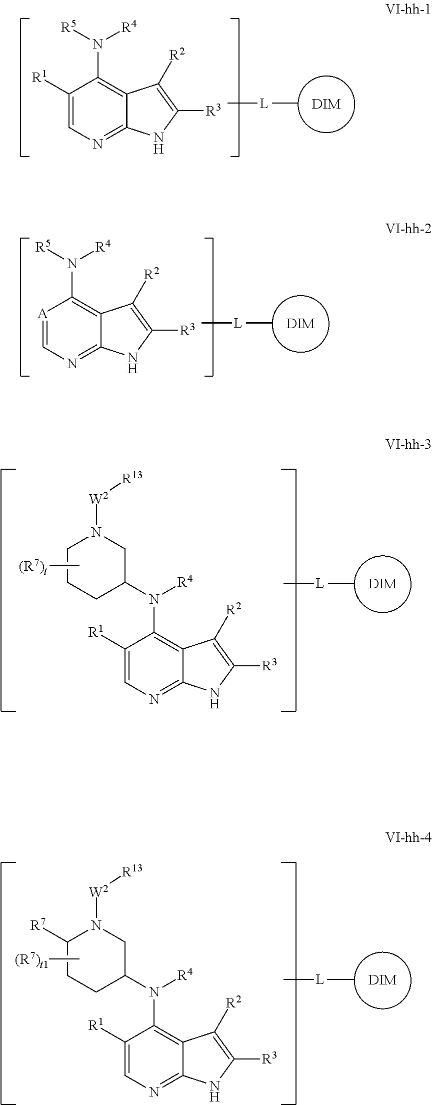
or a pharmaceutically acceptable salt thereof, wherein each of the variables R¹, R², R³, R⁴, R⁵, R⁷, R¹³, A, W², t, and t1, are as defined and described in U.S. Pat. No. 7,335,667;
(vi) 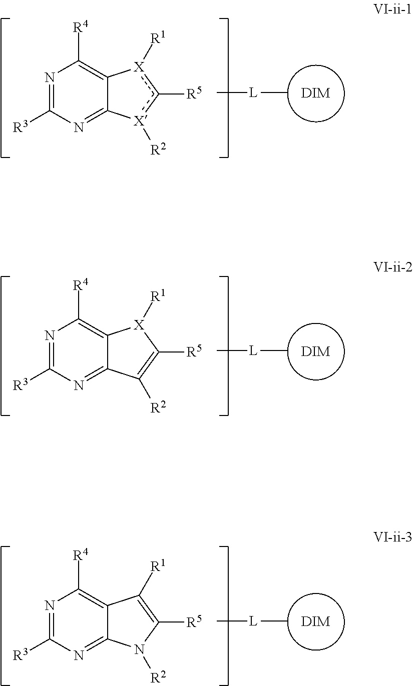
or a pharmaceutically acceptable salt thereof, wherein each of the variables R¹, R², R³, R⁴, R⁵, and X′ are as defined and described in U.S. pat. Nos. 9,555,031 and 9,273,056;
(vii) 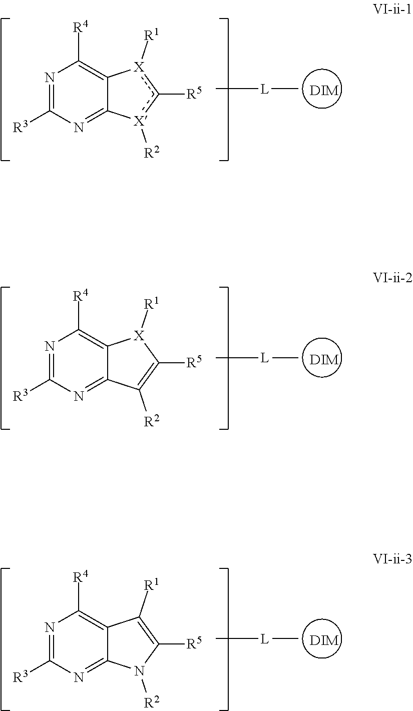
or a pharmaceutically acceptable salt thereof, wherein each of the variables R¹, R², R³, R⁴, R⁵, X and X′ are as defined and described in U.S. Pat. Nos. 9,555,031 and US 9,273,056;
(viii) 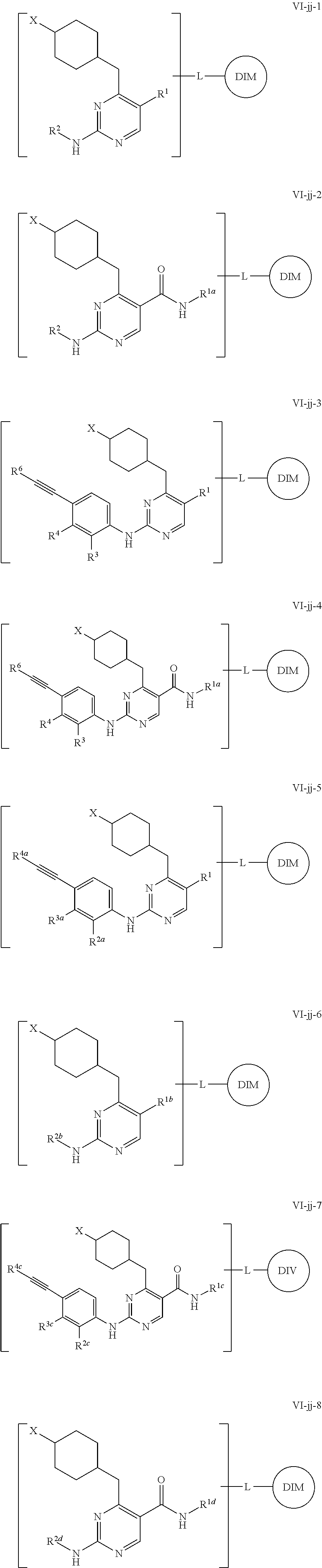
or a pharmaceutically acceptable salt thereof, wherein each of the variables X, R¹, R^{1a}, R^{1b}, R^{1c}, R^{1d}, R², R^{2a}, R^{2b}, R^{2c}, R^{2d}, R³, R^{3a}, R^{3c}, R⁴, R^{4a}, R^{4c}, and R⁶ are as defined and described in WO 2017/059280 and US 2018/0297977;
(ix) 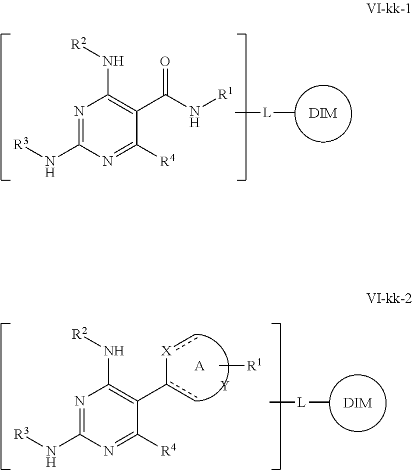
or a pharmaceutically acceptable salt thereof, wherein each of the variables R¹, R², R³, R⁴, Ring A, and Y are as defined and described in U.S. Pat. Nos. 9,567,326 and 9,771,330;
(x) 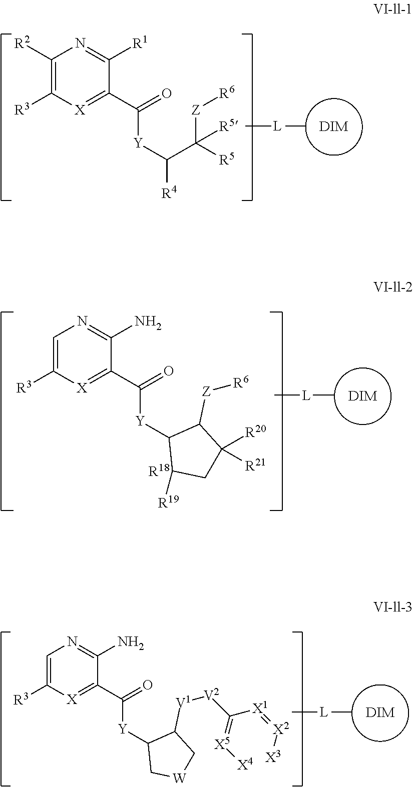
or a pharmaceutically acceptable salt thereof, wherein each of the variables R¹, R², R³, R⁴, R⁵, R^{5′}, R⁶, R¹⁸, R¹⁹, R²⁰, R²¹, V¹, V², X¹, X², X³, X⁴, X⁵, Y, and Z, are as defined and described in US 2017/0355690 and WO 2017/039331; 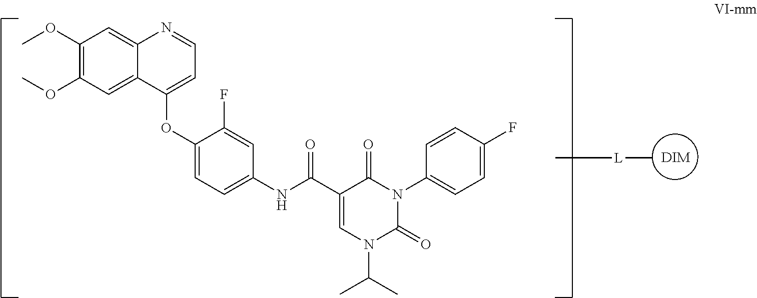
or a pharmaceutically acceptable salt thereof; or
(xi) 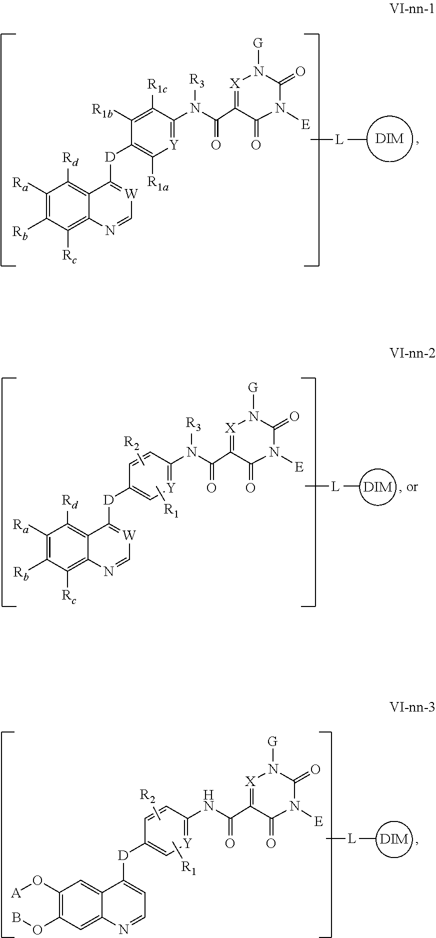
or a pharmaceutically acceptable salt thereof, wherein each of the variables R₁, R₂, R₃, R₁ₐ, R_{1b}, R_{1c}, Rₐ, R_{b}, R_{c}, R_{d}, D, E, G, X, and W, are as defined and described in U.S. Pat. No. 9,120,778.

17. The compound according to claim 16, wherein the TAMBM binding moiety is selected from:

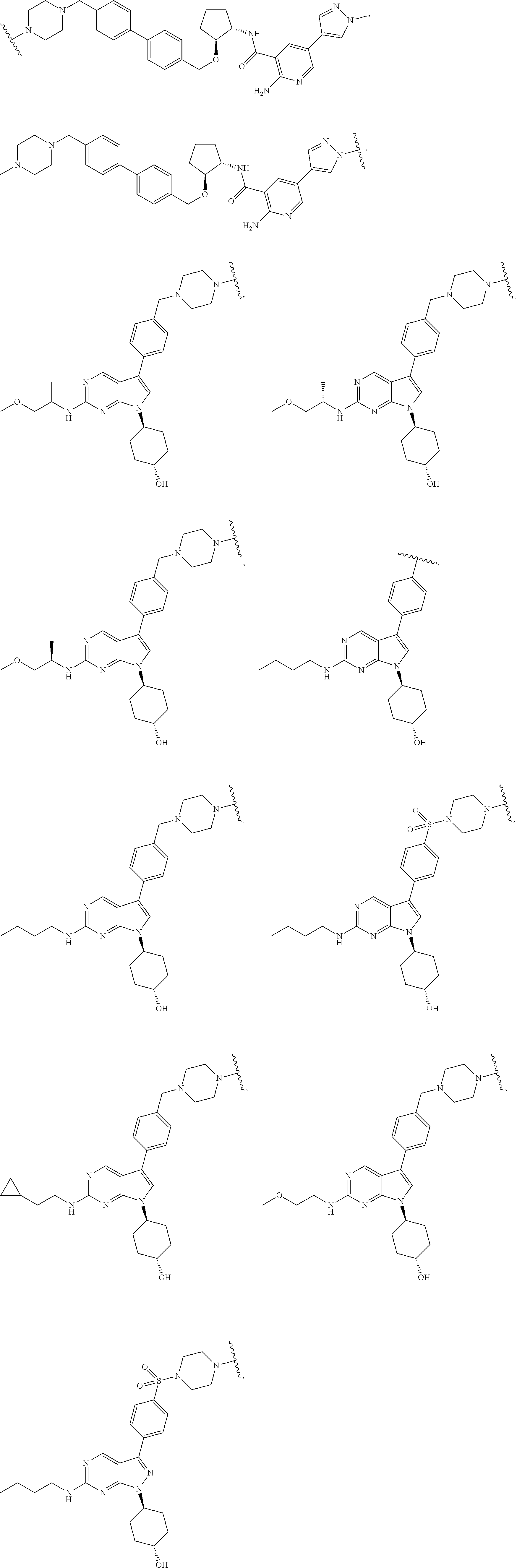

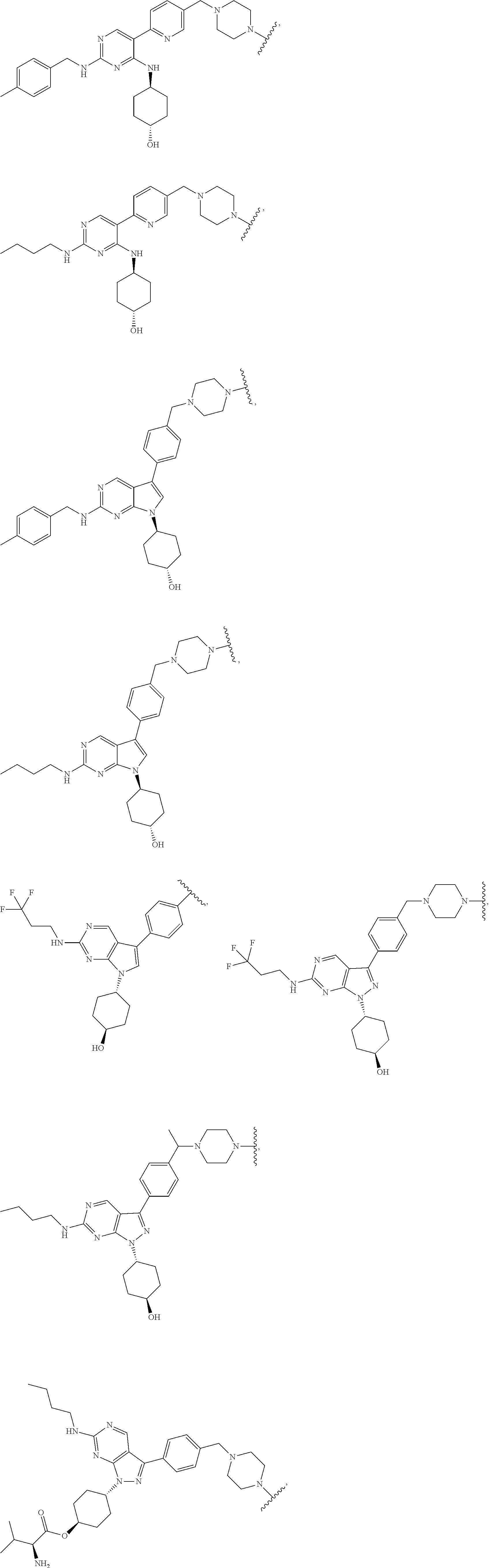

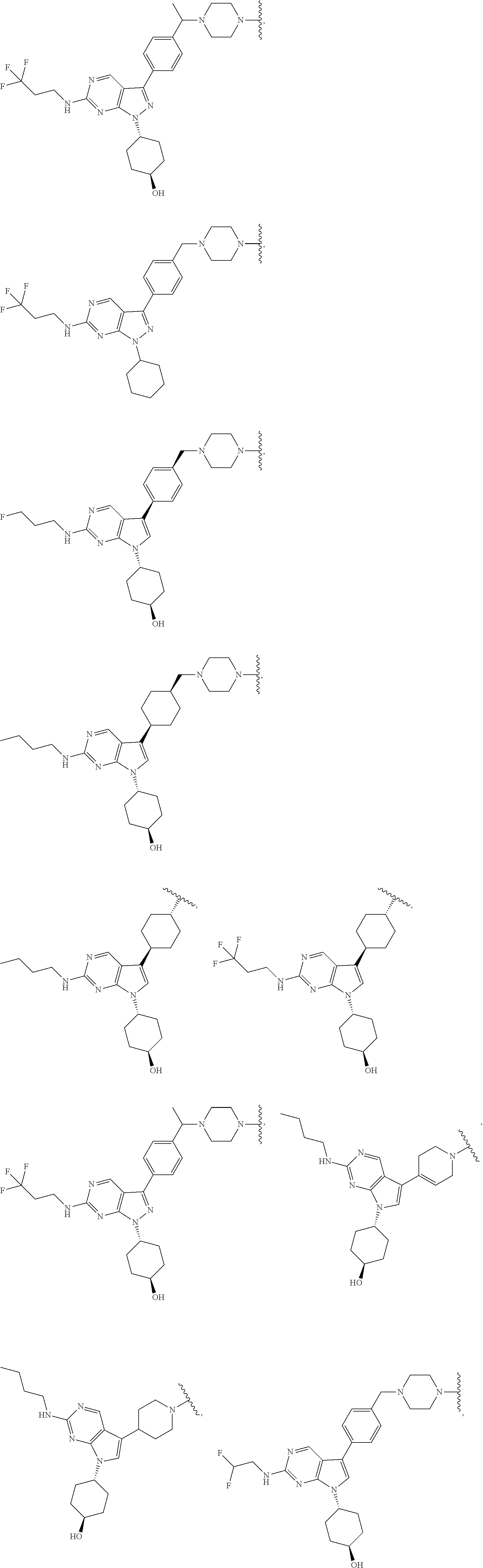

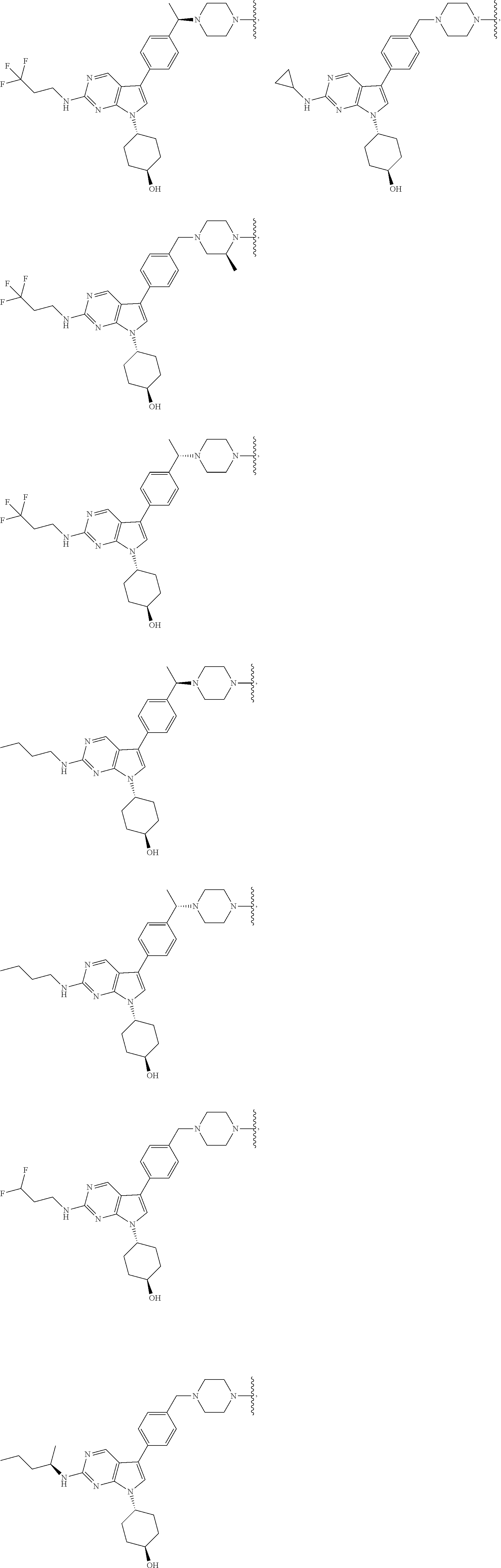

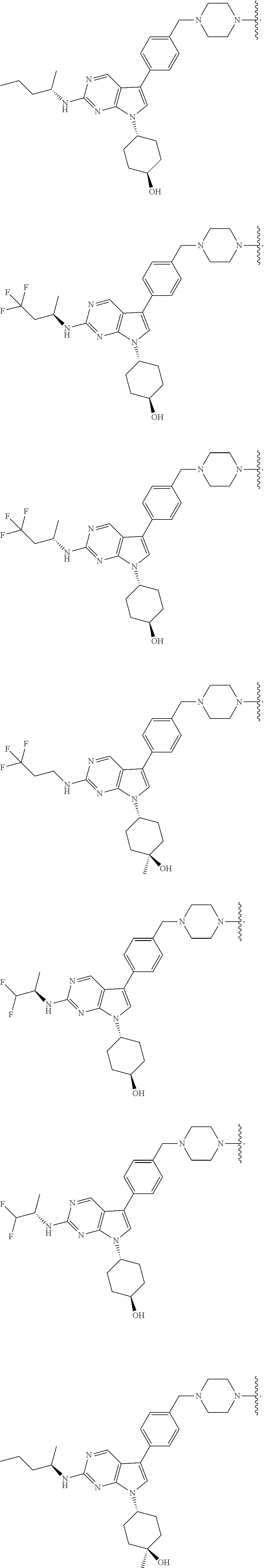

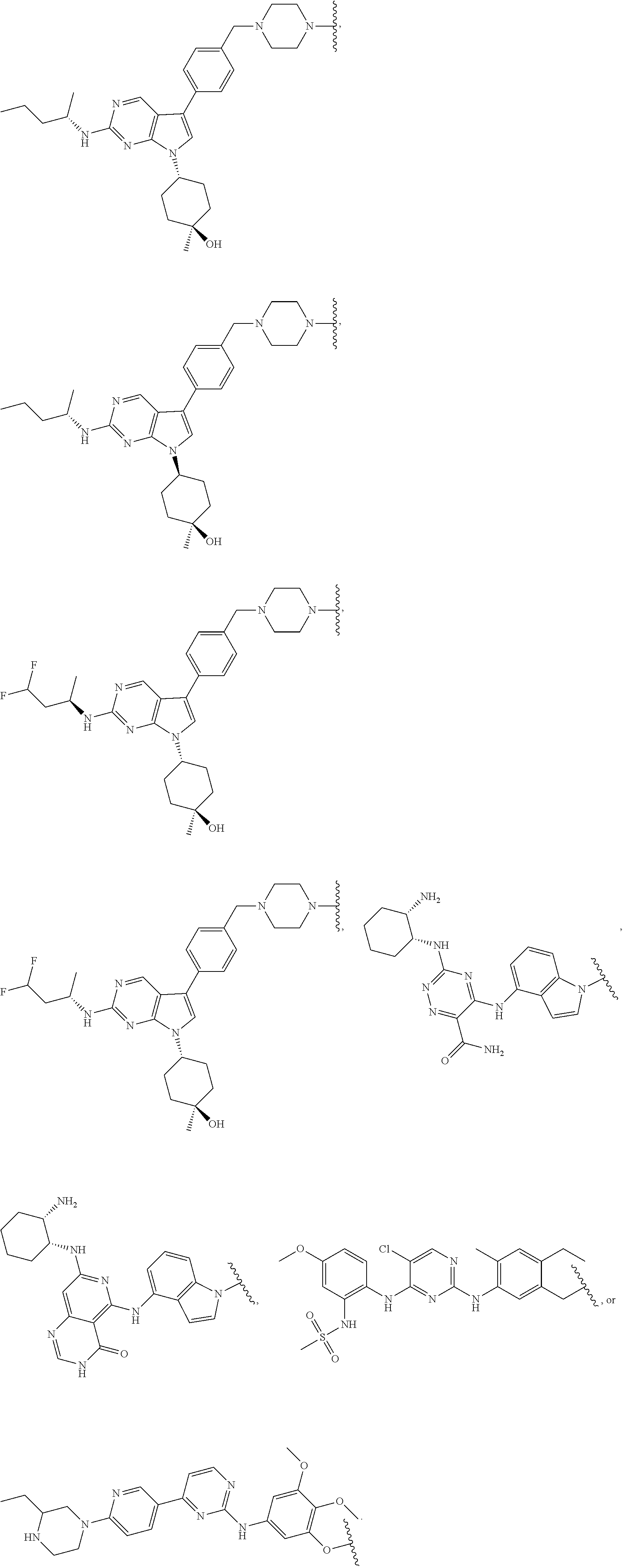

18. The compound of claim 1, wherein L is a covalent bond or a bivalent, saturated or unsaturated, straight or branched C₁₋₅₀ hydrocarbon chain, wherein 0-6 methylene units of L are independently replaced by -Cy-, —O—, —NR—, —S—, —OC(O)—, —C(O)O—, —C(O)—, —S(O)—, —S(O)₂—, —NRS(O)₂—, —S(O)₂NR—, —NRC(O)—, —C(O)NR—, —OC(O)NR—, —NRC(O)O—, 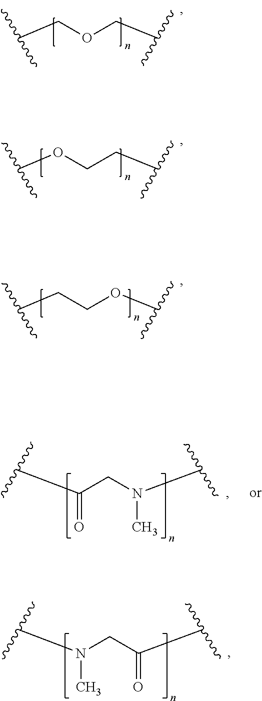
wherein: each -Cy- is independently an optionally substituted bivalent ring selected from phenylenyl, an 8-10 membered bicyclic arylenyl, a 4-7 membered saturated or partially unsaturated carbocyclylenyl, a 4-7 membered saturated or partially unsaturated spiro carbocyclylenyl, an 8-10 membered bicyclic saturated or partially unsaturated carbocyclylenyl, a 4-7 membered saturated or partially unsaturated heterocyclylenyl having 1-2 heteroatoms independently selected from nitrogen, oxygen, and sulfur, a 4-7 membered saturated or partially unsaturated spiro heterocyclylenyl having 1-2 heteroatoms independently selected from nitrogen, oxygen, and sulfur, an 8-10 membered bicyclic saturated or partially unsaturated heterocyclylenyl having 1-2 heteroatoms independently selected from nitrogen, oxygen, and sulfur, a 5-6 membered heteroarylenyl having 1-4 heteroatoms independently selected from nitrogen, oxygen, and sulfur, or an 8-10 membered bicyclic heteroarylenyl having 1-5 heteroatoms independently selected from nitrogen, oxygen, or sulfur; and wherein n is 0, 1, 2, 3, 4, 5, 6, 7, 8, 9, or 10.

19. The compound of any one of claims 1-18, wherein said compound is selected from any one of the compounds depicted in Table 1, or a pharmaceutically acceptable salt thereof.

20. A pharmaceutical composition comprising a compound according to claim 1-19, and a pharmaceutically acceptable carrier, adjuvant, or vehicle.

21. A method of degrading and/or inhibiting a TAM receptor kinase in a patient or biological sample comprising administering to said patient, or contacting said biological sample with a compound according to any one of claims 1-19, or a pharmaceutical composition thereof.

22. The method of claim 21, wherein the TAM receptor kinase is MerTK.

23. A method of treating a TAM receptor kinase-mediated disorder, disease, or condition in a patient comprising administering to said patient a compound according to any one of claims 1-19, or a pharmaceutical composition thereof.

24. The method of claim 23, wherein the TAM receptor kinase-mediate disorder, disease, or condition is a MerTK disorder, disease, or condition.

25. The method of claim 24, wherein the MerTK-mediated disorder, disease or condition is selected from the group consisting of an infectious disease, an immune disorder, an autoimmune disorder, an inflammatory disorder, a proliferation disorder, or a platelet aggregation disorder.

26. The method of claim 25, wherein the disorder is a proliferative disorder.

27. The method of claim 26, wherein the proliferative disorder is cancer.

28. The method of claim 27, wherein the cancer is selected from: melanoma; lung adenocarcinoma, basal cell carcinoma, squamous cell carcinoma, large cell carcinoma, bronchioloalveolar carcinoma, bronchiogenic carcinoma, non-small-cell carcinoma, small cell carcinoma, mesothelioma; breast cancer; ductal carcinoma, lobular carcinoma, inflammatory breast cancer, clear cell carcinoma, mucinous carcinoma, serosal cavities breast carcinoma; colorectal cancer; colon cancer, rectal cancer, colorectal adenocarcinoma; anal cancer; pancreatic cancer; pancreatic adenocarcinoma, islet cell carcinoma, neuroendocrine tumors; prostate cancer; prostate adenocarcinoma; ovarian carcinoma;., ovarian epithelial carcinoma or surface epithelial-stromal tumor; serous tumor, endometrioid tumor and mucinous cystadenocarcinoma, sex-cord-stromal tumor; liver and bile duct carcinoma; hepatocellular carcinoma, cholangiocarcinoma, hemangioma; esophageal carcinoma; esophageal adenocarcinoma and squamous cell carcinoma; oral and oropharyngeal squamous cell carcinoma; salivary gland adenoid cystic carcinoma; bladder cancer; bladder carcinoma; carcinoma of the uterus, endometrial adenocarcinoma, ocular, uterine papillary serous carcinoma, uterine clear-cell carcinoma, uterine sarcomas, and leiomyosarcomas, mixed mullerian tumors; glioma, glioblastoma, medulloblastoma, and other tumors of the brain; kidney cancers; renal cell carcinoma, clear cell carcinoma, Wilm's tumor; cancer of the head and neck; squamous cell carcinomas; cancer of the stomach; gastric cancers, stomach adenocarcinoma, gastrointestinal stromal tumor; testicular cancer; germ cell tumor; neuroendocrine tumor; cervical cancer; carcinoids of the gastrointestinal tract, breast, and other organs; signet ring cell carcinoma; mesenchymal tumors, sarcomas; fibrosarcomas, haemangioma, angiomatosis, haemangiopericytoma, pseudoangiomatous stromal hyperplasia, myofibroblastoma, fibromatosis, inflammatory myofibroblastic tumor, lipoma, angiolipoma, granular cell tumor, neurofibroma, schwannoma, angiosarcoma, liposarcoma, rhabdomyosarcoma, osteosarcoma, leiomyoma, leiomysarcoma, skin cancer; melanoma; cervical cancer; retinoblastoma; head and neck cancer; pancreatic cancer; brain cancer; thyroid cancer; testicular cancer; renal cancer; bladder cancer; soft tissue cancer; adrenal gland cancer; urethral cancer; cancers of the penis; myxosarcoma; chondrosarcoma; osteosarcoma; chordoma; malignant fibrous histiocytoma; lymphangiosarcoma; mesothelioma; squamous cell carcinoma; epidermoid carcinoma; malignant skin adnexal tumors; adenocarcinoma; hepatoma; hepatocellular carcinoma; renal cell carcinoma; hypernephroma; cholangiocarcinoma; transitional cell carcinoma; choriocarcinoma;, seminoma; embryonal cell carcinoma; glioma anaplastic; glioblastoma multiforme; neuroblastoma; medulloblastoma; malignant meningioma; malignant schwannoma; neurofibrosarcoma; parathyroid carcinoma; medullary carcinoma of thyroid; bronchial carcinoid; pheochromocytoma; Islet cell carcinoma; malignant carcinoid; malignant paraganglioma; melanoma; Merkel cell neoplasm; cystosarcoma phylloide, salivary cancers, thymic carcinomas; and cancers of the vagina.

29. The method of claim 27, wherein the cancer is selected from: AIDS-Related Lymphoma; Anaplastic Large-Cell Lymphoma; Angioimmunoblastic Lymphoma; Blastic NK-Cell Lymphoma; Burkitt's Lymphoma; Burkitt-like Lymphoma (Small Non-Cleaved Cell Lymphoma); Chronic Lymphocytic Leukemia/Small Lymphocytic Lymphoma; Cutaneous T-Cell Lymphoma; Diffuse Large B-Cell Lymphoma; Enteropathy-Type T-Cell Lymphoma; Follicular Lymphoma; Hepatosplenic Gamma-Delta T-Cell Lymphoma; Lymphoblastic Lymphoma; Mantle Cell Lymphoma; Marginal Zone Lymphoma; Nasal T-Cell Lymphoma; Pediatric Lymphoma; Peripheral T-Cell Lymphomas; Primary Central Nervous System Lymphoma; T-Cell Leukemias; Transformed Lymphomas; Treatment-Related T-Cell Lymphomas; or Waldenstrom's Macroglobulinemia.

30. The method of claim 25, wherein platelet aggregation disorder is a blood clot.

31. The method of claim 30, wherein the blood clot is a result of: coronary artery disease, peripheral vascular disease, cerebrovascular disease, stable and unstable angina pectoris, left ventricular dysfunction, congestive heart failure, myocardial death, myocardial infarction, atrial fibrillation, stroke, renal damage, percutaneous translumenal coronary angioplasty, athreosclerosis, disseminated intravascular coagulation, sepsis, endotoxemia, pulmonary embolism, and deep vein thrombosis.
